(19) United States
(12) Patent Application Publication
(10) Pub. No.: US 2024/0175019 A1
(43) Pub. Date: May 30, 2024

(54) Skeletal Muscle Delivery Platforms and Methods of Use

(71) Applicant: Arrowhead Pharmaceuticals, Inc., Pasadena, CA (US)

(72) Inventors: Xiaokai Li, Middleton, WI (US); Tao Pei, Middleton, WI (US); Teng Ai, Middleton, WI (US); Susan Phan, Fitchburg, WI (US); Susan Ramos-Hunter, Hendersonville, TN (US); Andrei V. Blokhin, Fitchburg, WI (US)

(21) Appl. No.: 18/181,335

(22) Filed: Mar. 9, 2023

Related U.S. Application Data
(63) Continuation of application No. PCT/US2021/049874, filed on Sep. 10, 2021.
(60) Provisional application No. 63/077,284, filed on Sep. 11, 2020.

(51) Int. Cl. C12N 15/113; A61K 47/54; A61K 47/60

(52) U.S. Cl. CPC C12N 15/113; A61K 47/543; A61K 47/545; A61K 47/60; C12N 2310/14; C12N 2310/351; C12N 2310/3515

(57) ABSTRACT

The present disclosure relates to delivery platforms that specifically and efficiently direct payloads to skeletal muscle cells in a subject, in vivo. The delivery platforms disclosed herein include targeting ligands (such as compounds that have affinity for integrins, including alpha-v-beta-6) and pharmacokinetic/pharmacodynamic (PK/PD) modulators, to facilitate the delivery of payloads to cells, including to skeletal muscle cells. Suitable payloads for use in the delivery platforms disclosed herein include RNAi agents, which can be linked or conjugated to the delivery platforms, and when delivered in vivo, provide for the inhibition of gene expression in skeletal muscle cells. Pharmaceutical compositions that include the skeletal muscle cell delivery platform are also described, as well as methods of use for the treatment of various diseases and disorders where delivery of a therapeutic payload to a skeletal muscle cell is desirable.

### CROSS REFERENCE TO RELATED APPLICATIONS

This application is a continuation application under 35 U.S.C. 111(a) of PCT application PCT/US2021/049874, filed Sep. 10, 2021, which claims the benefit of U.S. provisional application No. 63/077,284, filed on Sep. 11, 2020. These documents are hereby incorporated by reference in their entirety.

### SEQUENCE LISTING

This application contains a Sequence Listing which has been submitted in XML format and is hereby incorporated by reference in its entirety. The XML copy is named 30689-US1_ST26_SeqListing.xml, created Mar. 8, 2023, and is 96 kb in size.

### FIELD OF THE INVENTION

The present disclosure relates to delivery platforms for the delivery of payloads, such as RNA interference (RNAi) agents, e.g., double stranded RNAi agents, to skeletal muscle cells in vivo. The delivery of RNAi agents using the delivery platforms disclosed herein provide for the inhibition of genes that are expressed in skeletal muscle cells.

### BACKGROUND OF THE INVENTION

Directing therapeutic or diagnostic payloads to specific tissues of interest in vivo in a subject continues to be a great challenge in the field of medicine. This includes achieving specific and selective delivery to skeletal muscle cells, where various diseases and disorders find their origin. The inability to selectively and efficiently deliver payloads, such as therapeutic drug products, to skeletal muscle cells prevents many diseases and disorders from being properly treated and addressed.

For decades now, oligonucleotide-based therapeutics, such as for example antisense oligonucleotide compounds (ASOs) and double-stranded RNA interference (RNAi) agents, have shown great promise and the potential to revolutionize the field of medicine and provide for potent therapeutic treatment options. However, the delivery of oligonucleotide-based therapeutics, and double-stranded therapeutic RNAi agents in particular, has long been a challenge in developing viable therapeutic pharmaceutical agents. This is particularly the case when trying to achieve specific and selective delivery of oligonucleotide-based therapeutics to non-hepatocyte cells, such as skeletal muscle cells.

While various attempts over the past several years have been made to direct oligonucloide-based therapeutics to skeletal muscle cells, using, for example, cholesterol conjugates (which is non-specific and has the known disadvantage of distributing to various undesired tissues and organs) and lipid-nanoparticles (LNPs) (which have been frequently reported to have toxicity concerns), to date none have achieved suitable delivery. There remains a need for a delivery mechanism or platform to specifically and efficiently direct oligonucleotide-based therapeutics, and RNAi agents in particular, to skeletal muscle cells.

### SUMMARY

Disclosed herein is a delivery platform that directs payloads, such as oligonucleotide-based therapeutics including RNA interference (RNAi) agents (also herein termed RNAi agent, RNAi trigger, or trigger; e.g., double-stranded RNAi agents), to skeletal muscle cells and facilitate the selective and efficient inhibition of the expression of genes present in skeletal muscle cells. Further disclosed herein are compositions that include an RNAi agent for inhibiting expression of target genes, wherein the RNAi agent is linked to at least one targeting ligand that has affinity for a cell receptor present on a targeted cell, and, optionally, at least one pharmacokinetic and/or pharmacodynamic (PK/PD) modulator. The RNAi agents disclosed herein can selectively and efficiently decrease or inhibit expression of a target gene in a subject, e.g., a human or animal subject.

The described RNAi agents can be used in methods for therapeutic treatment (including prophylactic, intervention, and preventative treatment) of conditions and diseases that can be mediated at least in part by the reduction in target gene expression, including, for example, muscular dystrophy, including Duchenne Muscular Dystrophy, Becker Muscular Dystrophy, myotonic muscular dystrophy, and Facioscapulohumeral (FSHD). The RNAi agents disclosed herein can selectively reduce target gene expression in cells in a subject. The methods disclosed herein include the administration of one or more RNAi agents to a subject, e.g., a human or animal subject, using any suitable methods known in the art, such as intravenous infusion, intravenous injection, or subcutaneous injection.

Also described herein are pharmaceutical compositions that include an RNAi agent capable of inhibiting the expression of a target gene, wherein the composition further includes at least one pharmaceutically acceptable excipient. The pharmaceutical compositions described herein that include one or more of the disclosed RNAi agents are able to selectively and efficiently decrease or inhibit expression of a target gene in vivo. The compositions that include one or more RNAi agents can be administered to a subject, such as a human or animal subject, for the treatment (including prophylactic treatment or inhibition) of conditions and diseases that can be mediated at least in part by a reduction in target gene expression, including, for example, muscular dystrophy.

One aspect described herein is a delivery platform composition for inhibiting expression of a gene expressed in skeletal muscle cells comprising:
a. An RNAi agent comprising:
   i. An antisense strand comprising 17-49 nucleotides wherein at least 15 nucleotides are complementary to the mRNA sequence of a gene that is expressed in skeletal muscle cells
   ii. A sense strand that is 16-49 nucleotides in length that is at least partially complementary to the antisense strand;
b. A targeting ligand with affinity for a receptor present on the surface of a skeletal muscle cell; and
c. A PK/PD modulator;
   wherein the RNAi agent is covalently linked to the targeting ligand and to the PK/PD modulator.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying FIGURES, and from the claims.

### DETAILED DESCRIPTION

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a target mRNA.

As used herein, an “oligonucleotide-based agent” is a nucleotide sequence containing about 10-50 (e.g., 10 to 48, 10 to 46, 10 to 44, 10 to 42, 10 to 40, 10 to 38, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 50, 12 to 48, 12 to 46, 12 to 44, 12 to 42, 12 to 40, 12 to 38, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 50, 14 to 48, 14 to 46, 14 to 44, 14 to 42, 14 to 40, 14 to 38, 14 to 36, 14 to 34, 14 to 32, 14 to 30, 14 to 28, 14 to 26, 14 to 24, 14 to 22, 14 to 20, 14 to 18, 14 to 16, 16 to 50, 16 to 48, 16 to 46, 16 to 44, 16 to 42, 16 to 40, 16 to 38, 16 to 36, 16 to 34, 16 to 32, 16 to 30, 16 to 28, 16 to 26, 16 to 24, 16 to 22, 16 to 20, 16 to 18, 18 to 50, 18 to 48, 18 to 46, 18 to 44, 18 to 42, 18 to 40, 18 to 38, 18 to 36, 18 to 34, 18 to 32, 18 to 30, 18 to 28, 18 to 26, 18 to 24, 18 to 22, 18 to 20, 20 to 50, 20 to 48, 20 to 46, 20 to 44, 20 to 42, 20 to 40, 20 to 38, 20 to 36, 20 to 34, 20 to 32, 20 to 30, 20 to 28, 20 to 26, 20 to 24, 20 to 22, 22 to 50, 22 to 48, 22 to 46, 22 to 44, 22 to 42, 22 to 40, 22 to 38, 22 to 36, 22 to 34, 22 to 32, 22 to 30, 22 to 28, 22 to 26, 22 to 24, 24 to 50, 24 to 48, 24 to 46, 24 to 44, 24 to 42, 24 to 40, 24 to 38, 24 to 36, 24 to 34, 24 to 32, 24 to 30, 24 to 28, 24 to 26, 26 to 50, 26 to 48, 26 to 46, 26 to 44, 26 to 42, 26 to 40, 26 to 38, 26 to 36, 26 to 34, 26 to 32, 26 to 30, 26 to 28, 28 to 50, 28 to 48, 28 to 46, 28 to 44, 28 to 42, 28 to 40, 28 to 38, 28 to 36, 28 to 34, 28 to 32, to 28 to 30, 30 to 50, 30 to 48, 30 to 46, 30 to 44, 30 to 42, 30 to 40, 30 to 38, 30 to 36, 30 to 34, 30 to 32, 32 to 50, 32 to 48, 32 to 46, 32 to 44, 32 to 42, 32 to 40, 32 to 38, 32 to 36, 32 to 34, 34 to 50, 34 to 48, 34 to 46, 34 to 44, 34 to 42, 34 to 40, 34 to 38, 34 to 36, 36 to 50, 36 to 48, 36 to 46, 36 to 44, 36 to 42, 36 to 40, 36 to 38, 38 to 50, 38 to 48, 38 to 46, 38 to 44, 38 to 42, 38 to 40, 40 to 50, 40 to 48, 40 to 46, 40 to 44, 40 to 42, 42 to 50, 42 to 48, 42 to 46, 42 to 44, 44 to 50, 44 to 48, 44 to 46, 46 to 50, 46 to 48, or 48 to 50) nucleotides or nucleotide base pairs. In some embodiments, an oligonucleotide-based agent has a nucleobase sequence that is at least partially complementary to a coding sequence in an expressed target nucleic acid or target gene within a cell. In some embodiments, the oligonucleotide-based agent, upon delivery to a cell expressing a gene, are able to inhibit the expression of the underlying gene, and are referred to herein as “expression-inhibiting oligonucleotide-based agents.” The gene expression can be inhibited in vitro or in vivo.

“Oligonucleotide-based agents” include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates. In some embodiments, an oligonucleotide-based agent is a single-stranded oligonucleotide, such as an antisense oligonucleotide. In some embodiments, an oligonucleotide-based agent is a double-stranded oligonucleotide. In some embodiments, an oligonucleotide-based agent is a double-stranded oligonucleotide that is an RNAi agent.

As used herein and as would be understood by one skilled in the art, a polyethylene glycol (PEG) unit refers to repeating units of the formula —(CH₂CH₂O)—. It will be appreciated that, in the chemical structures disclosed herein, PEG units may be depicted as —(CH₂CH₂O)—, —(OCH₂CH₂)—, or —(CH₂OCH₂)—. It will also be appreciated that a numeral indicating the number of repeating PEG units may be placed on either side of the parentheses depicting the PEG units. It will be further appreciated that a terminal PEG unit may be end capped by an atom (e.g., a hydrogen atom) or some other moiety.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the preventative treatment, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

As used herein, the term “lipid” refers to moieties and molecules that are soluble in nonpolar solvents. The term lipid includes amphiphilic molecules comprising a polar, water-soluble head group and a hydrophobic tail. Lipids can be of natural or synthetic origin. Non-limiting examples of lipids include fatty acids (e.g., saturated fatty acids, monounsaturated fatty acids, and polyunsatured fatty acids), glycerolipids (e.g., monoacylglycerols, diacylglycerols, and triacylglycerols), phospholipids (e.g., phosphatidylethanolamine, phosphatidylcholine, and phosphatidylserine), sphingolipids (e.g., sphingomyelin), and cholesterol esters. As used herein, the term “saturated lipid” refers to lipids that are free of any unsaturation. As used herein, the term “unsaturated lipid” refers to lipids that comprise at least one (1) degree of unsaturation. As used herein, the term “branched lipid” refers to lipids comprising more than one linear chain, wherein each liner chain is covalently attached to at least one other linear chain. As used herein, the term “straight chain lipid” refers to lipids that are free of any branching.

As used herein, the term “linked” or “conjugated” when referring to the connection between two compounds or molecules means that two molecules are joined by a covalent bond or are associated via noncovalent bonds (e.g., hydrogen bonds or ionic bonds). In some examples, where the term “linked” or “conjugated” refers to the association between two molecules via noncovalent bonds, the association between the two different molecules has a K_{D} of less than 1×10⁻⁴ M (e.g., less than 1×10⁻⁵ M, less than 1×10⁻⁶ M, or less than 1×10⁻⁷ M) in physiologically acceptable buffer (e.g., buffered saline). Unless stated, the terms “linked” and “conjugated” as used herein may refer to the connection between a first compound and a second compound either with or without any intervening atoms or groups of atoms.

As used herein, a linking group is one or more atoms that connects one molecule or portion of a molecule to another to second molecule or second portion of a molecule. Similarly, as used in the art, the term scaffold is sometimes used interchangeably with a linking group. Linking groups may comprise any number of atoms or functional groups. In some embodiments, linking groups may not facilitate any biological or pharmaceutical response, and merely serve to link two biologically active molecules.

Unless stated otherwise, the symbol

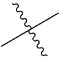

as used herein means that any group or groups may be linked thereto that is in accordance with the scope of the inventions described herein.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

### Modified Nucleotides

In some embodiments, an RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as N_{UNA} or NUNA), locked nucleotides (represented herein as N_{LNA} or NLNA), 3′-O-methoxy (2′ internucleoside linked) nucleotides (represented herein as 3′-OMen), 2′-F-Arabino nucleotides (represented herein as NfANA or Nf_{ANA}), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (also referred to herein as 2′-fluoro nucleotide, and represented herein as NO, 2′-deoxy nucleotides (represented herein as dN), 2′-methoxyethyl (2′-O-2-methoxylethyl) nucleotides (also referred to herein as 2′-MOE, and represented herein as NM), 2′-amino nucleotides, and 2′-alkyl nucleotides. It is not necessary for all positions in a given compound to be uniformly modified. Conversely, more than one modification can be incorporated in a single RNAi agent or even in a single nucleotide thereof. The RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine , 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide.

### Modified Internucleoside Linkages

In some embodiments, one or more nucleotides of an RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′.

In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an RNAi agent can contain 1, 2, 3, 4, 5, or 6 phosphorothioate linkages, an antisense strand of an RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an RNAi agent can contain 1, 2, 3, or 4 phosphorothioate linkages, an antisense strand of an RNAi agent can contain 1, 2, 3, or 4 phosphorothioate linkages, or both the sense strand and the antisense strand independently can contain 1, 2, 3, or 4 phosphorothioate linkages.

In some embodiments, an RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, one phosphorothioate internucleoside linkage is at the 5′ end of the sense strand, and another phosphorothioate linkage is at the 3′ end of the sense strand. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, an RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, an RNAi agent contains at least three or four phosphorothioate internucleoside linkages in the antisense strand.

In some embodiments, an RNAi agent contains one or more modified nucleotides and one or more modified internucleoside linkages. In some embodiments, a 2′-modified nucleoside is combined with modified internucleoside linkage.

### Targeting Ligands and Targeting Groups

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers. In some embodiments, a targeting group comprises an integrin targeting ligand.

In some embodiments, RNAi agents described herein are conjugated to targeting groups. In some embodiments, a targeting ligand enhances the ability of the RNAi agent to bind to a particular cell receptor on a cell of interest. In some embodiments, the targeting ligands conjugated to RNAi agents described herein have affinity for integrin receptors. In some embodiments, a suitable targeting ligand for use with the RNAi agents disclosed herein has affinity for integrin alpha-v-beta 6. Targeting groups comprise two or more targeting ligands.

In some embodiments, a delivery platform disclosed herein comprises one or more integrin targeting ligands that include a compound of the formula:

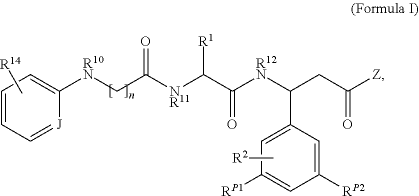

or a pharmaceutically acceptable salt thereof,
wherein,
   n is an integer from 0 to 7;
   J is C—H or N;
   Z is OR¹³, N(R¹³)₂ or SR¹³;
R¹ is H, optionally substituted C₁-C₆ alkyl, OH, COOH, CON(R⁵)₂, OR⁶, or R¹ comprises a cargo molecule, wherein each R⁵ is independently H or C₁-C₆ alkyl, and R⁶ is H or C₁-C₆ alkyl;
R², R^{P1} and R^{P2} are each independently H, halo, optionally substituted cycloalkylene, optionally substituted arylene, optionally substituted heterocycloalkylene, or optionally substituted heteroarylene, or R², R^{P1} and R^{P2} may comprise a cargo molecule;
R¹⁰ is H or optionally substituted alkyl;
R¹¹ is H or optionally substituted alkyl, or R¹¹ and R¹ together with the atoms to which they are attached form an optionally substituted heterocycle;
R¹² is H or optionally substituted alkyl;
each R¹³ is independently H, optionally substituted alkyl, or R¹³ comprises a cargo molecule;
R¹⁴ is optionally substituted alkyl; and
wherein at least one of R¹, R², R¹³, R^{P1} and R^{P2} comprises the antisense strand.

Methods of making compounds of Formula I are described in PCT Patent Publication No. WO 2019/089765, which is herein incorporated by reference in its entirety, and in example 3, below.

In some embodiments, compounds that may be conjugated to RNAi agents to synthesize a delivery platform for an RNAi agent are shown in Table 1 below.

TABLE 1
Compounds that may be conjugated to RNAi agents to synthesize a delivery platform for an RNAi agent.

| | |
|---|---|
| Structure 1b | 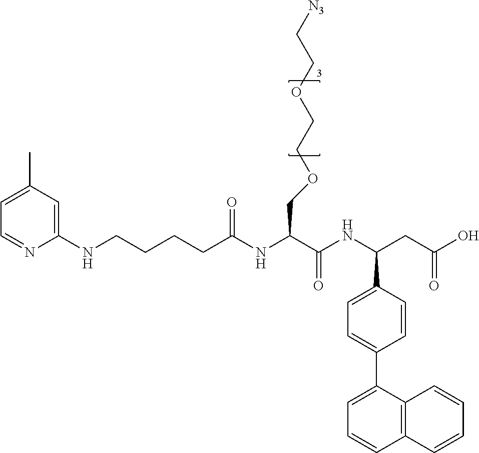 |
| Structure 2b | 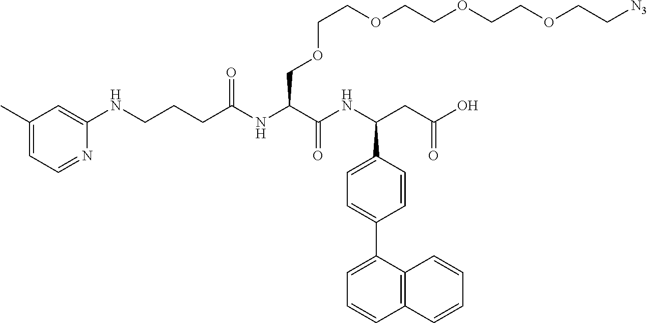 [SMILES: Cc1ccnc(NCCCC(=O)N[C@@H](COCCOCCOCCOCCN=[N+]=[N-])C(=O)N[C@@H](CC(=O)O)c2ccc(-c3cccc4ccccc34)cc2)c1] |
| Structure 5b | 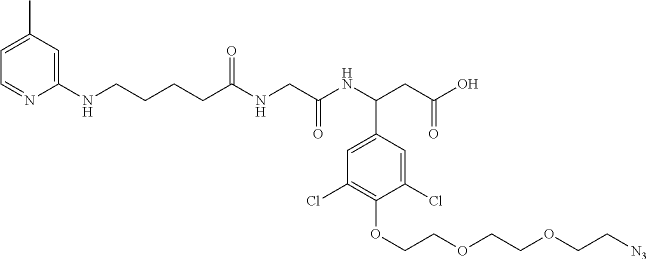 [SMILES: Cc1ccnc(NCCCCC(=O)NCC(=O)NC(CC(=O)O)c2cc(Cl)c(OCCOCCOCCN=[N+]=[N-])c(Cl)c2)c1] |
| Structure 5.1b | 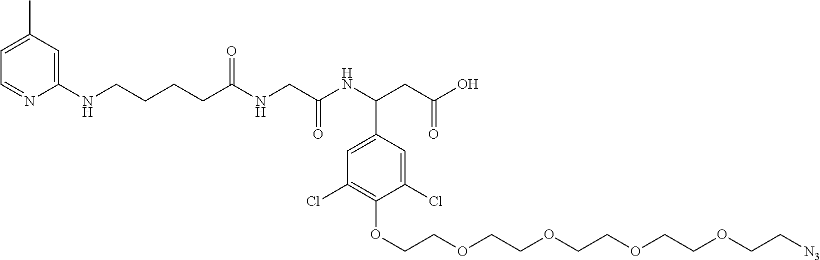 [SMILES: Cc1ccnc(NCCCCC(=O)NCC(=O)NC(CC(=O)O)c2cc(Cl)c(OCCOCCOCCOCCOCCN=[N+]=[N-])c(Cl)c2)c1] |
| Structure 5.2b | 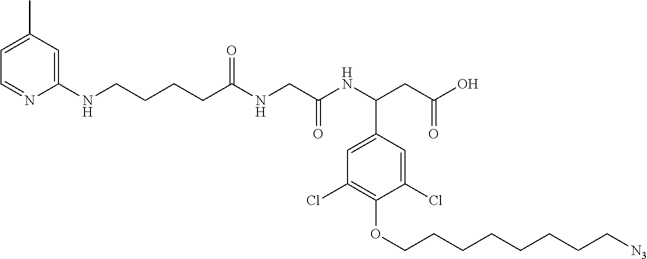 [SMILES: Cc1ccnc(NCCCCC(=O)NCC(=O)NC(CC(=O)O)c2cc(Cl)c(OCCCCCCCCN=[N+]=[N-])c(Cl)c2)c1] |
| Structure 6b | 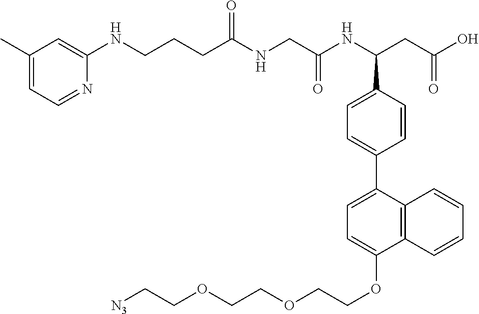 [SMILES: Cc1ccnc(NCCCC(=O)NCC(=O)N[C@@H](CC(=O)O)c2ccc(-c3ccc(OCCOCCOCCN=[N+]=[N-])c4ccccc34)cc2)c1] |
| Structure 6.1b | 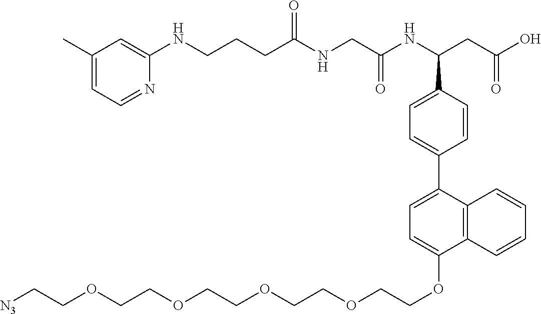 [SMILES: Cc1ccnc(NCCCC(=O)NCC(=O)N[C@@H](CC(=O)O)c2ccc(-c3ccc(OCCOCCOCCOCCOCCN=[N+]=[N-])c4ccccc34)cc2)c1] |
| Structure 6.2b | 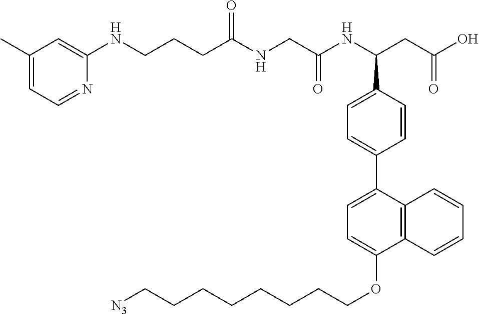 [SMILES: Cc1ccnc(NCCCC(=O)NCC(=O)N[C@@H](CC(=O)O)c2ccc(-c3ccc(OCCCCCCCCN=[N+]=[N-])c4ccccc34)cc2)c1] |
| Structure 6.3b | 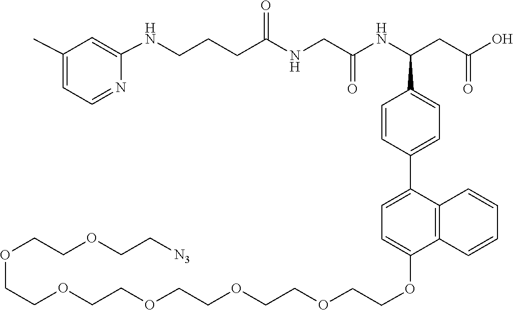 [SMILES: Cc1ccnc(NCCCC(=O)NCC(=O)N[C@@H](CC(=O)O)c2ccc(-c3ccc(OCCOCCOCCOCCOCCOCCOCCN=[N+]=[N-])c4ccccc34)cc2)c1] |
| Structure 6.4b | 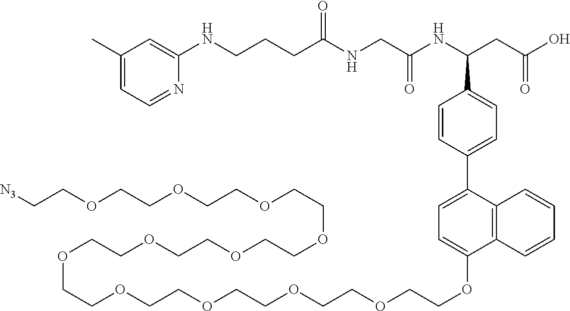 [SMILES: Cc1ccnc(NCCCC(=O)NCC(=O)N[C@@H](CC(=O)O)c2ccc(-c3ccc(OCCOCCOCCOCCOCCOCCOCCOCCOCCOCCOCCOCCN=[N+]=[N-])c4ccccc34)cc2)c1] |
| Structure 7b | 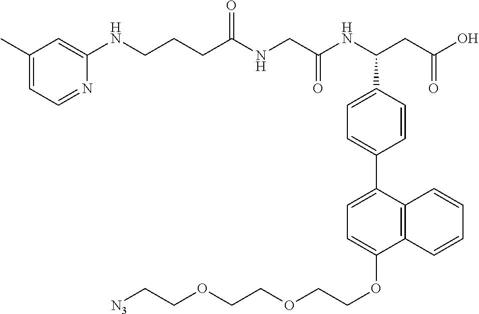 [SMILES: Cc1ccnc(NCCCC(=O)NCC(=O)N[C@H](CC(=O)O)c2ccc(-c3ccc(OCCOCCOCCN=[N+]=[N-])c4ccccc34)cc2)c1] |
| Structure 8b | 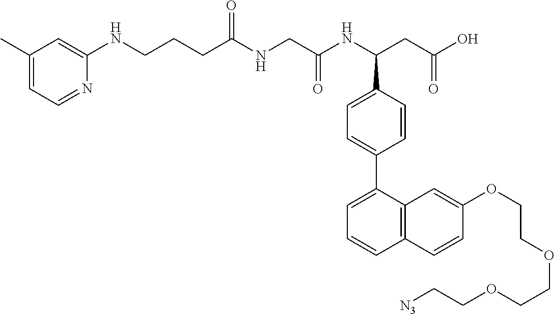 [SMILES: Cc1ccnc(NCCCC(=O)NCC(=O)N[C@@H](CC(=O)O)c2ccc(-c3cccc4ccc(OCCOCCOCCN=[N+]=[N-])cc34)cc2)c1] |
| Structure 9b | 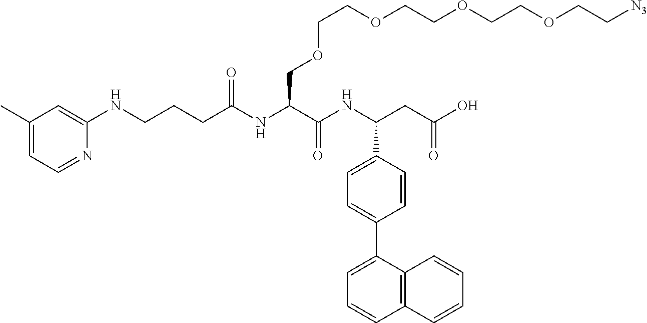 [SMILES: Cc1ccnc(NCCCC(=O)N[C@@H](COCCOCCOCCOCCN=[N+]=[N-])C(=O)N[C@H](CC(=O)O)c2ccc(-c3cccc4ccccc34)cc2)c1] |
| Structure 10b | 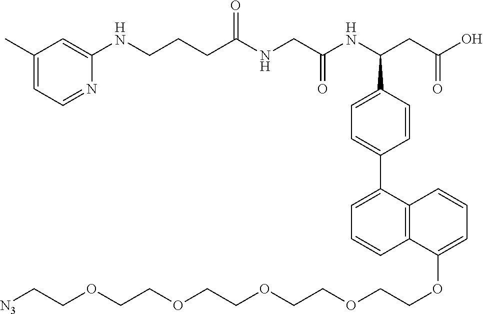 [SMILES: Cc1ccnc(NCCCC(=O)NCC(=O)N[C@@H](CC(=O)O)c2ccc(-c3cccc4c(OCCOCCOCCOCCOCCN=[N+]=[N-])cccc34)cc2)c1] |
| Structure 11b | 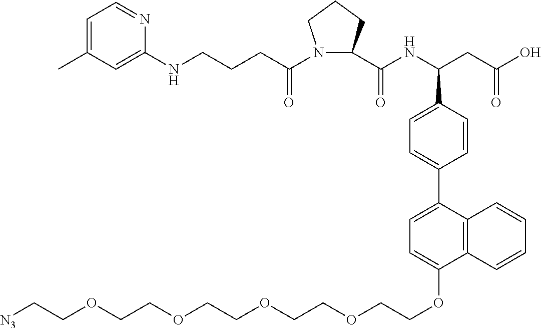 [SMILES: Cc1ccnc(NCCCC(=O)N2CCC[C@H]2C(=O)N[C@@H](CC(=O)O)c2ccc(-c3ccc(OCCOCCOCCOCCOCCN=[N+]=[N-])c4ccccc34)cc2)c1] |
| Structure 12b | 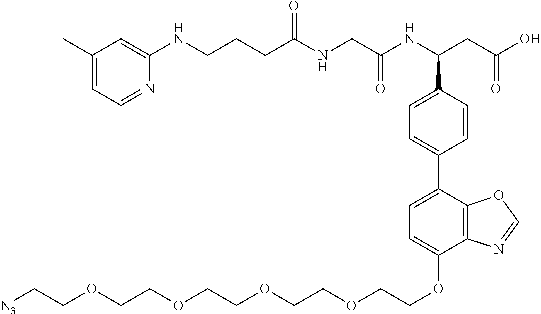 [SMILES: Cc1ccnc(NCCCC(=O)NCC(=O)N[C@@H](CC(=O)O)c2ccc(-c3ccc(OCCOCCOCCOCCOCCN=[N+]=[N-])c4ncoc34)cc2)c1] |
| Structure 13b | 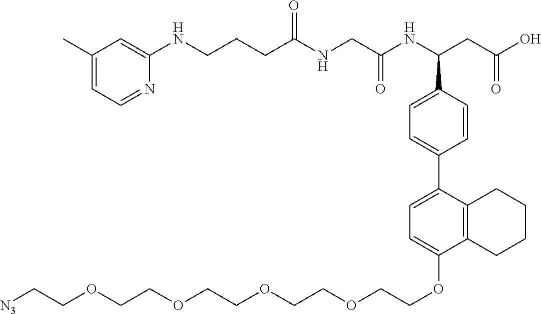 [SMILES: Cc1ccnc(NCCCC(=O)NCC(=O)N[C@@H](CC(=O)O)c2ccc(-c3ccc(OCCOCCOCCOCCOCCN=[N+]=[N-])c4c3CCCC4)cc2)c1] |
| Structure 14b | 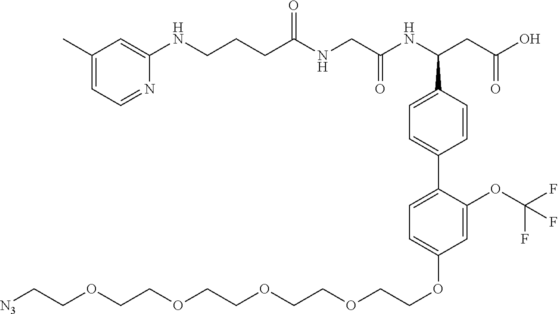 [SMILES: Cc1ccnc(NCCCC(=O)NCC(=O)N[C@@H](CC(=O)O)c2ccc(-c3ccc(OCCOCCOCCOCCOCCN=[N+]=[N-])cc3OC(F)(F)F)cc2)c1] |
| Structure 15b | 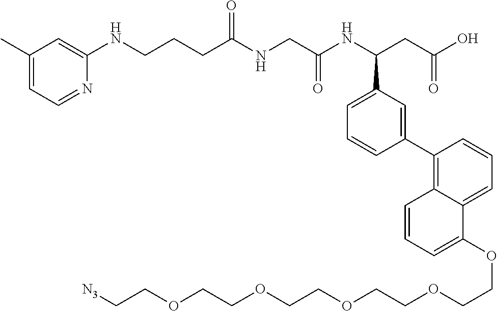 [SMILES: Cc1ccnc(NCCCC(=O)NCC(=O)N[C@@H](CC(=O)O)c2cccc(-c3cccc4c(OCCOCCOCCOCCOCCN=[N+]=[N-])cccc34)c2)c1] |
| Structure 16b | 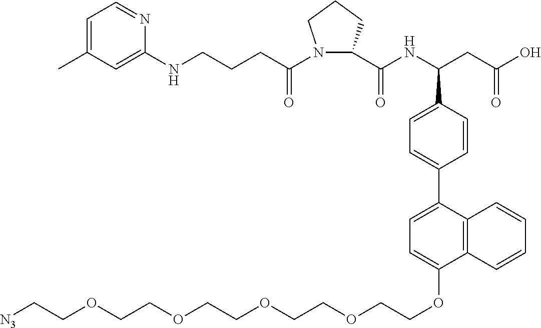 [SMILES: Cc1ccnc(NCCCC(=O)N2CCC[C@@H]2C(=O)N[C@@H](CC(=O)O)c2ccc(-c3ccc(OCCOCCOCCOCCOCCN=[N+]=[N-])c4ccccc34)cc2)c1] |
| Structure 17b | 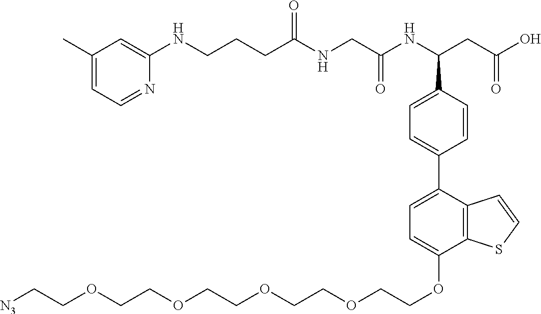 [SMILES: Cc1ccnc(NCCCC(=O)NCC(=O)N[C@@H](CC(=O)O)c2ccc(-c3ccc(OCCOCCOCCOCCOCCN=[N+]=[N-])c4sccc34)cc2)c1] |
| Structure 18b | 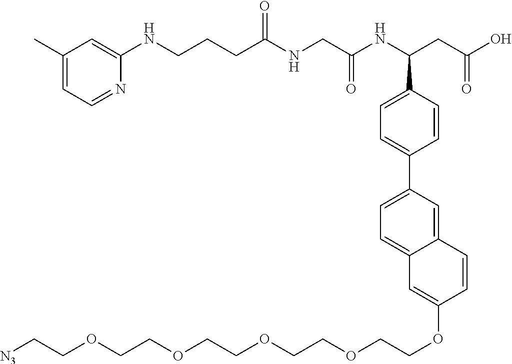 [SMILES: Cc1ccnc(NCCCC(=O)NCC(=O)N[C@@H](CC(=O)O)c2ccc(-c3ccc4cc(OCCOCCOCCOCCOCCN=[N+]=[N-])ccc4c3)cc2)c1] |
| Structure 19b | 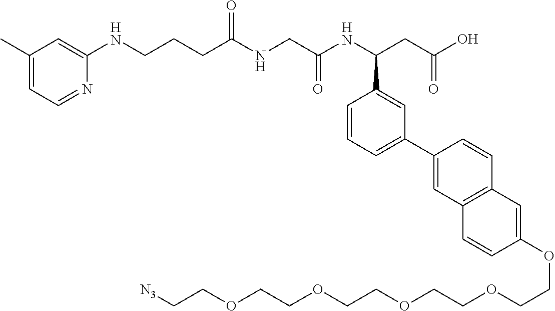 [SMILES: Cc1ccnc(NCCCC(=O)NCC(=O)N[C@@H](CC(=O)O)c2cccc(-c3ccc4cc(OCCOCCOCCOCCOCCN=[N+]=[N-])ccc4c3)c2)c1] |
| Structure 20b | 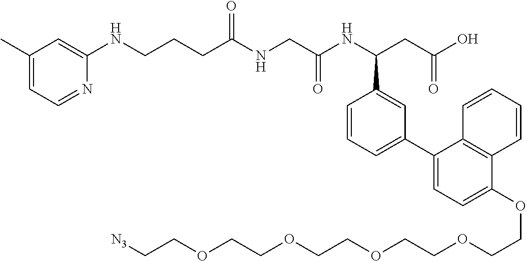 [SMILES: Cc1ccnc(NCCCC(=O)NCC(=O)N[C@@H](CC(=O)O)c2cccc(-c3ccc(OCCOCCOCCOCCOCCN=[N+]=[N-])c4ccccc34)c2)c1] |
| Structure 22b | 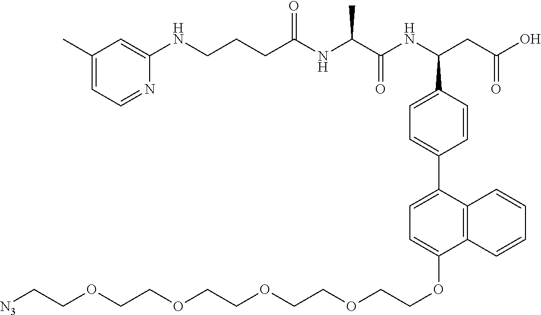 [SMILES: Cc1ccnc(NCCCC(=O)N[C@@H](C)C(=O)N[C@@H](CC(=O)O)c2ccc(-c3ccc(OCCOCCOCCOCCOCCN=[N+]=[N-])c4ccccc34)cc2)c1] |
| Structure 23b | 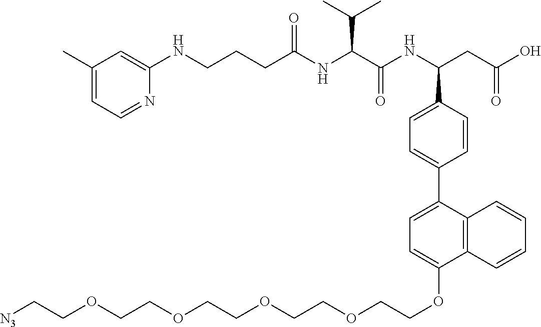 [SMILES: Cc1ccnc(NCCCC(=O)N[C@H](C(=O)N[C@@H](CC(=O)O)c2ccc(-c3ccc(OCCOCCOCCOCCOCCN=[N+]=[N-])c4ccccc34)cc2)C(C)C)c1] |
| Structure 24b | 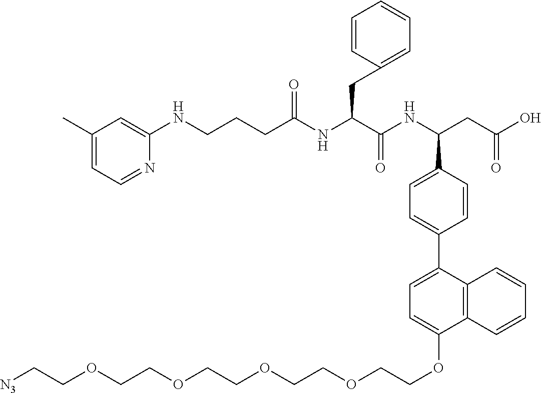 [SMILES: Cc1ccnc(NCCCC(=O)N[C@@H](Cc2ccccc2)C(=O)N[C@@H](CC(=O)O)c2ccc(-c3ccc(OCCOCCOCCOCCOCCN=[N+]=[N-])c4ccccc34)cc2)c1] |
| Structure 25b | 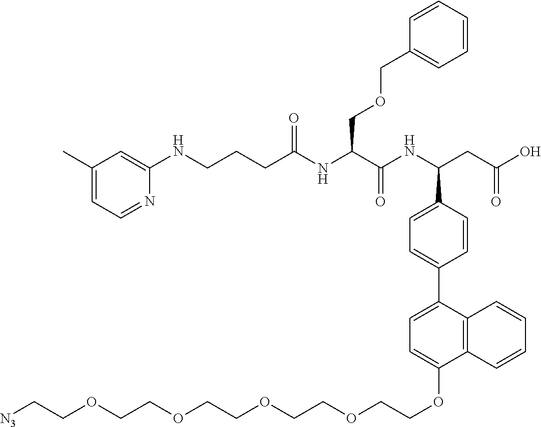 [SMILES: Cc1ccnc(NCCCC(=O)N[C@@H](COCc2ccccc2)C(=O)N[C@@H](CC(=O)O)c2ccc(-c3ccc(OCCOCCOCCOCCOCCN=[N+]=[N-])c4ccccc34)cc2)c1] |
| Structure 27b | 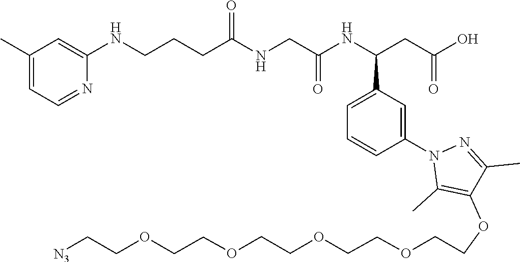 |
| Structure 29b | 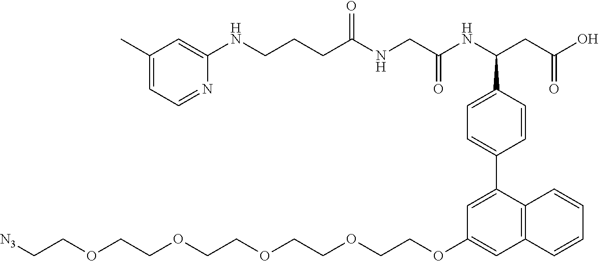 [SMILES: Cc1ccnc(NCCCC(=O)NCC(=O)N[C@@H](CC(=O)O)c2ccc(-c3cc(OCCOCCOCCOCCOCCN=[N+]=[N-])cc4ccccc34)cc2)c1] |
| Structure 30b | 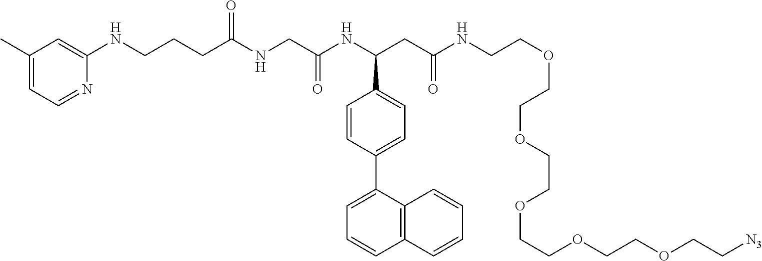 [SMILES: Cc1ccnc(NCCCC(=O)NCC(=O)N[C@@H](CC(=O)NCCOCCOCCOCCOCCOCCN=[N+]=[N-])c2ccc(-c3cccc4ccccc34)cc2)c1] |
| Structure 31b | 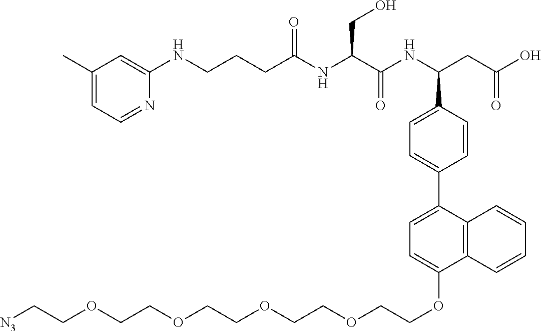 [SMILES: Cc1ccnc(NCCCC(=O)N[C@@H](CO)C(=O)N[C@@H](CC(=O)O)c2ccc(-c3ccc(OCCOCCOCCOCCOCCN=[N+]=[N-])c4ccccc34)cc2)c1] |
| Structure 32b | 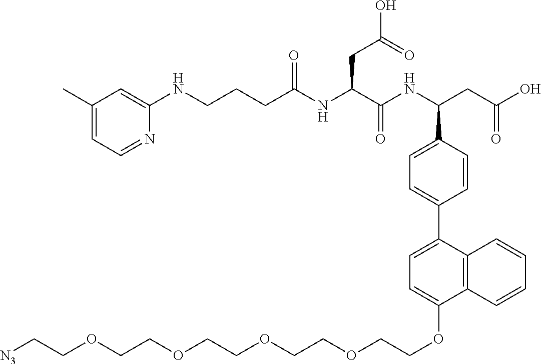 [SMILES: Cc1ccnc(NCCCC(=O)N[C@@H](CC(=O)O)C(=O)N[C@@H](CC(=O)O)c2ccc(-c3ccc(OCCOCCOCCOCCOCCN=[N+]=[N-])c4ccccc34)cc2)c1] |
| Structure 33b | 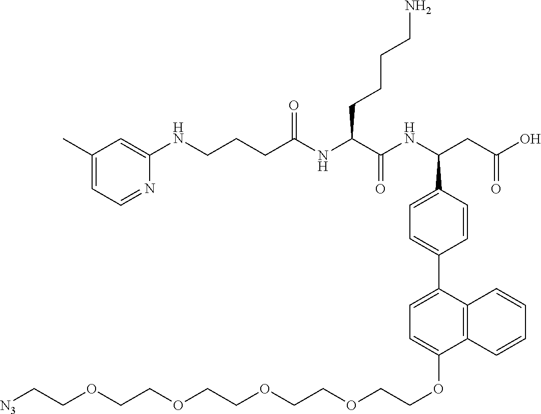 [SMILES: Cc1ccnc(NCCCC(=O)N[C@@H](CCCCN)C(=O)N[C@@H](CC(=O)O)c2ccc(-c3ccc(OCCOCCOCCOCCOCCN=[N+]=[N-])c4ccccc34)cc2)c1] |
| Structure 34b | 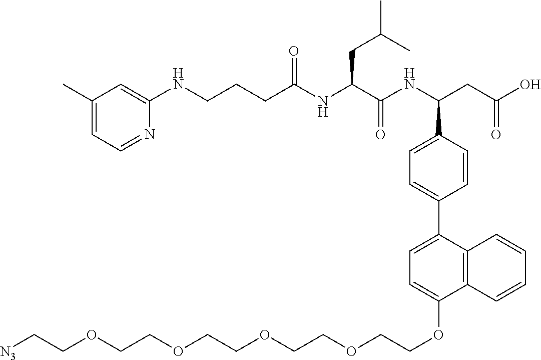 [SMILES: Cc1ccnc(NCCCC(=O)N[C@@H](CC(C)C)C(=O)N[C@@H](CC(=O)O)c2ccc(-c3ccc(OCCOCCOCCOCCOCCN=[N+]=[N-])c4ccccc34)cc2)c1] |
| Structure 35b | 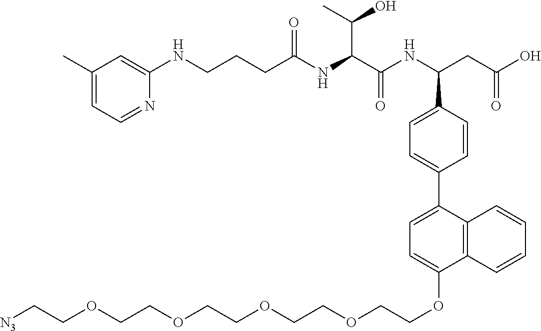 [SMILES: Cc1ccnc(NCCCC(=O)N[C@H](C(=O)N[C@@H](CC(=O)O)c2ccc(-c3ccc(OCCOCCOCCOCCOCCN=[N+]=[N-])c4ccccc34)cc2)[C@@H](C)O)c1] |
| Structure 36b | 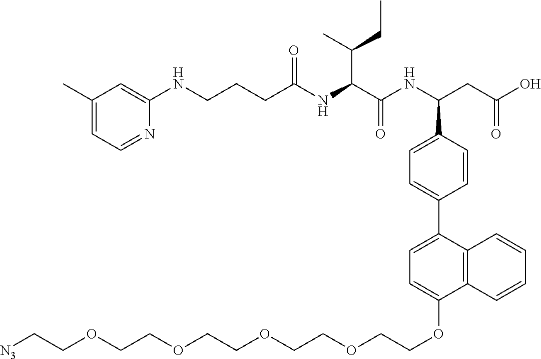 [SMILES: CC[C@H](C)[C@H](NC(=O)CCCNc1cc(C)ccn1)C(=O)N[C@@H](CC(=O)O)c1ccc(-c2ccc(OCCOCCOCCOCCOCCN=[N+]=[N-])c3ccccc23)cc1] |
| Structure 37b | 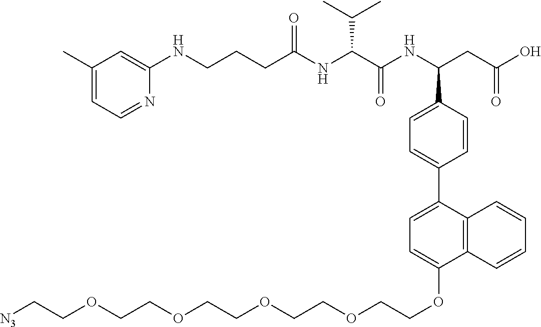 [SMILES: Cc1ccnc(NCCCC(=O)N[C@@H](C(=O)N[C@@H](CC(=O)O)c2ccc(-c3ccc(OCCOCCOCCOCCOCCN=[N+]=[N-])c4ccccc34)cc2)C(C)C)c1] |

or a pharmaceutically acceptable salt thereof.

In some embodiments, a delivery platform disclosed herein comprises one or more integrin targeting ligands that include one or more of the structures in Table 2 below.

TABLE 2
Integrin targeting ligands that may be conjugated to a delivery platform for RNAi agents.

| | |
|---|---|
| Structure 1 | 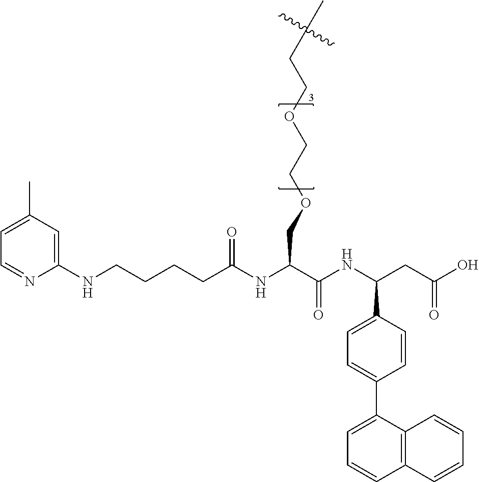 |
| Structure 2 | 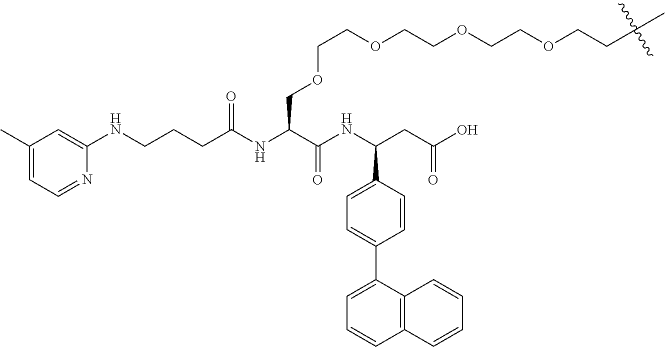 [SMILES: *CCOCCOCCOCCOC[C@H](NC(=O)CCCNc1cc(C)ccn1)C(=O)N[C@@H](CC(=O)O)c1ccc(-c2cccc3ccccc23)cc1] |
| Structure 5 | 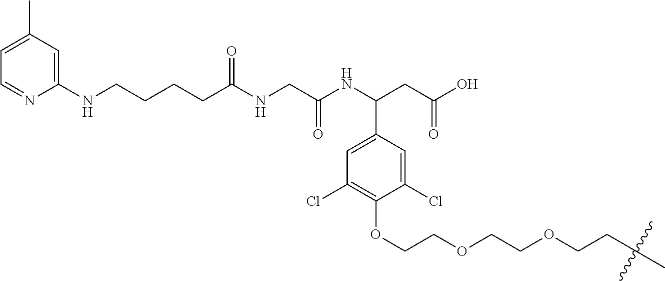 [SMILES: *CCOCCOCCOc1c(Cl)cc(C(CC(=O)O)NC(=O)CNC(=O)CCCCNc2cc(C)ccn2)cc1Cl] |
| Structure 5.1 | 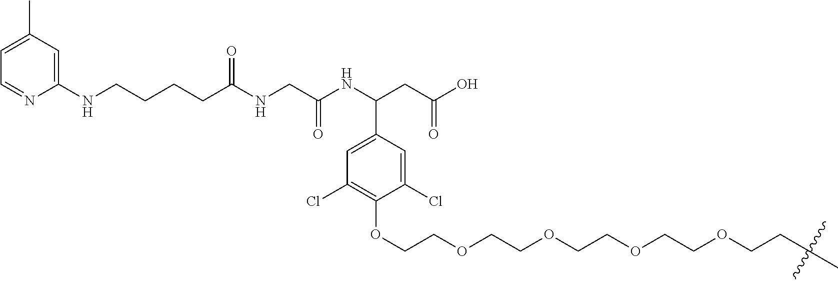 [SMILES: *CCOCCOCCOCCOCCOc1c(Cl)cc(C(CC(=O)O)NC(=O)CNC(=O)CCCCNc2cc(C)ccn2)cc1Cl] |
| Structure 5.2 | 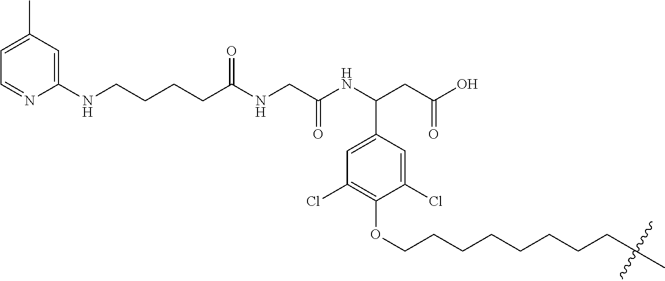 [SMILES: *CCCCCCCCOc1c(Cl)cc(C(CC(=O)O)NC(=O)CNC(=O)CCCCNc2cc(C)ccn2)cc1Cl] |
| Structure 6 | 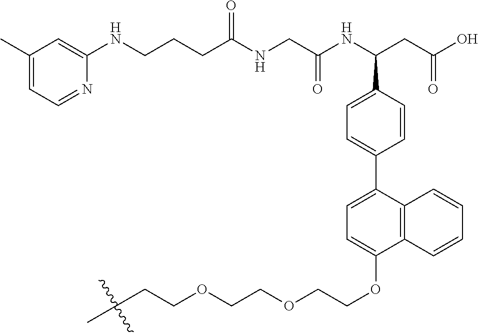 [SMILES: *CCOCCOCCOc1ccc(-c2ccc([C@H](CC(=O)O)NC(=O)CNC(=O)CCCNc3cc(C)ccn3)cc2)c2ccccc12] |
| Structure 6.1 | 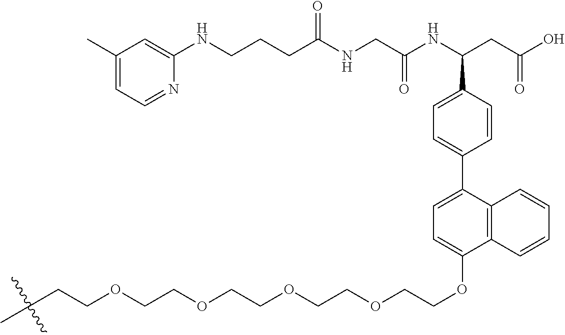 [SMILES: *CCOCCOCCOCCOCCOc1ccc(-c2ccc([C@H](CC(=O)O)NC(=O)CNC(=O)CCCNc3cc(C)ccn3)cc2)c2ccccc12] |
| Structure 6.2 | 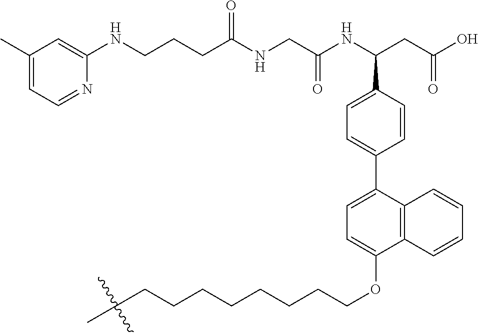 [SMILES: *CCCCCCCCOc1ccc(-c2ccc([C@H](CC(=O)O)NC(=O)CNC(=O)CCCNc3cc(C)ccn3)cc2)c2ccccc12] |
| Structure 6.3 | 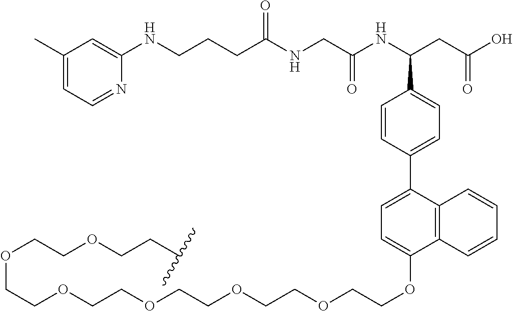 [SMILES: *CCOCCOCCOCCOCCOCCOCCOc1ccc(-c2ccc([C@H](CC(=O)O)NC(=O)CNC(=O)CCCNc3cc(C)ccn3)cc2)c2ccccc12] |
| Structure 6.4 | 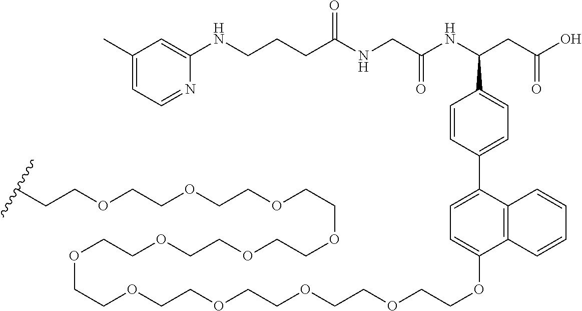 [SMILES: *CCOCCOCCOCCOCCOCCOCCOCCOCCOCCOCCOCCOc1ccc(-c2ccc([C@H](CC(=O)O)NC(=O)CNC(=O)CCCNc3cc(C)ccn3)cc2)c2ccccc12] |
| Structure 7 | 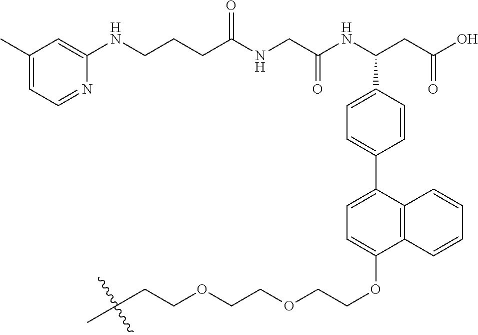 [SMILES: *CCOCCOCCOc1ccc(-c2ccc([C@@H](CC(=O)O)NC(=O)CNC(=O)CCCNc3cc(C)ccn3)cc2)c2ccccc12] |
| Structure 8 | 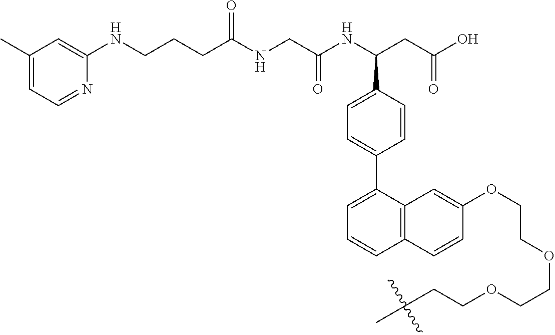 [SMILES: *CCOCCOCCOc1ccc2cccc(-c3ccc([C@H](CC(=O)O)NC(=O)CNC(=O)CCCNc4cc(C)ccn4)cc3)c2c1] |
| Structure 9 | 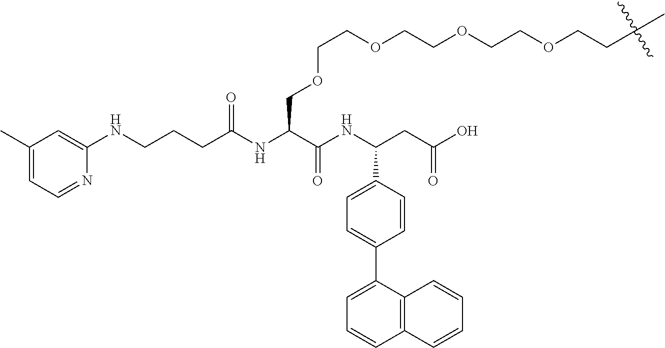 [SMILES: *CCOCCOCCOCCOC[C@H](NC(=O)CCCNc1cc(C)ccn1)C(=O)N[C@H](CC(=O)O)c1ccc(-c2cccc3ccccc23)cc1] |
| Structure 10 | 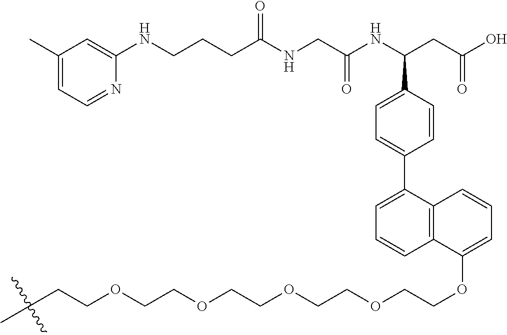 [SMILES: *CCOCCOCCOCCOCCOc1cccc2c(-c3ccc([C@H](CC(=O)O)NC(=O)CNC(=O)CCCNc4cc(C)ccn4)cc3)cccc12] |
| Structure 11 | 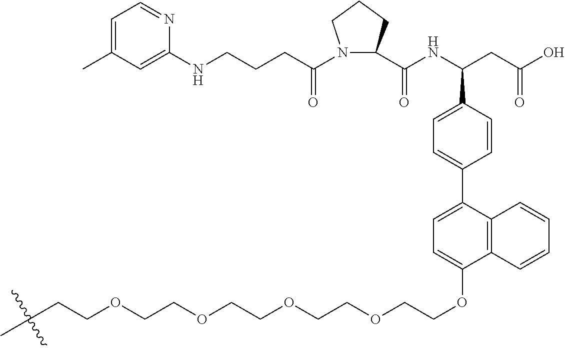 [SMILES: *CCOCCOCCOCCOCCOc1ccc(-c2ccc([C@H](CC(=O)O)NC(=O)[C@@H]3CCCN3C(=O)CCCNc3cc(C)ccn3)cc2)c2ccccc12] |
| Structure 12 | 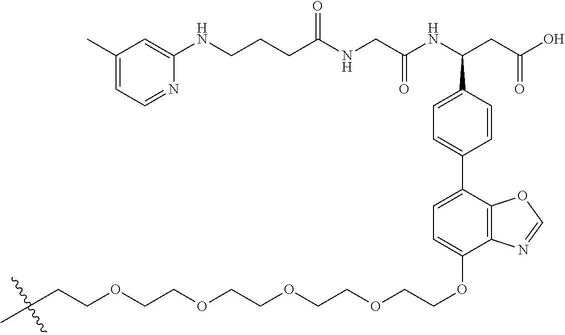 [SMILES: *CCOCCOCCOCCOCCOc1ccc(-c2ccc([C@H](CC(=O)O)NC(=O)CNC(=O)CCCNc3cc(C)ccn3)cc2)c2ocnc12] |
| Structure 13 | 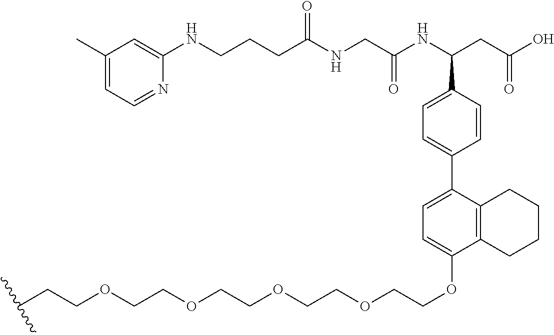 [SMILES: *CCOCCOCCOCCOCCOc1ccc(-c2ccc([C@H](CC(=O)O)NC(=O)CNC(=O)CCCNc3cc(C)ccn3)cc2)c2c1CCCC2] |
| Structure 14 | 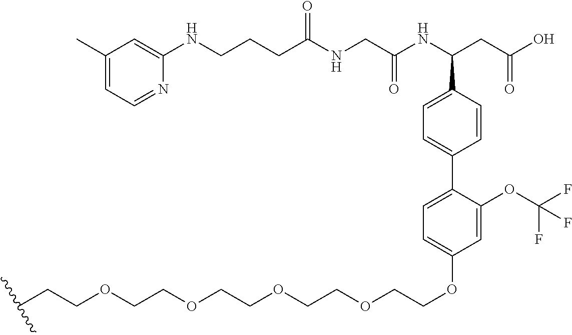 [SMILES: *CCOCCOCCOCCOCCOc1ccc(-c2ccc([C@H](CC(=O)O)NC(=O)CNC(=O)CCCNc3cc(C)ccn3)cc2)c(OC(F)(F)F)c1] |
| Structure 15 | 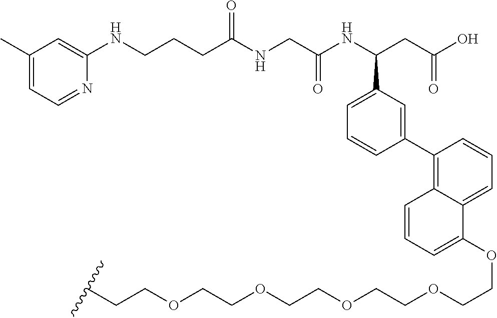 [SMILES: *CCOCCOCCOCCOCCOc1cccc2c(-c3cccc([C@H](CC(=O)O)NC(=O)CNC(=O)CCCNc4cc(C)ccn4)c3)cccc12] |
| Structure 16 | 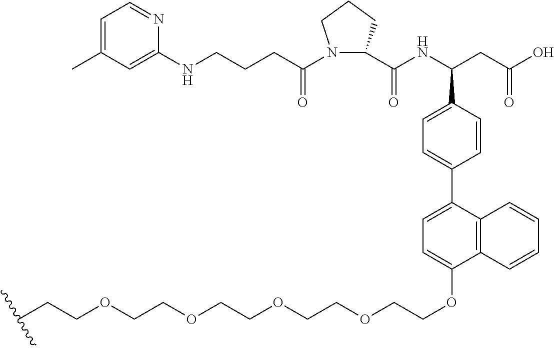 [SMILES: *CCOCCOCCOCCOCCOc1ccc(-c2ccc([C@H](CC(=O)O)NC(=O)[C@H]3CCCN3C(=O)CCCNc3cc(C)ccn3)cc2)c2ccccc12] |
| Structure 17 | 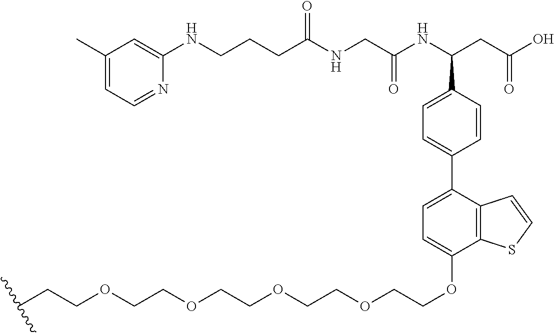 [SMILES: *CCOCCOCCOCCOCCOc1ccc(-c2ccc([C@H](CC(=O)O)NC(=O)CNC(=O)CCCNc3cc(C)ccn3)cc2)c2ccsc12] |
| Structure 18 | 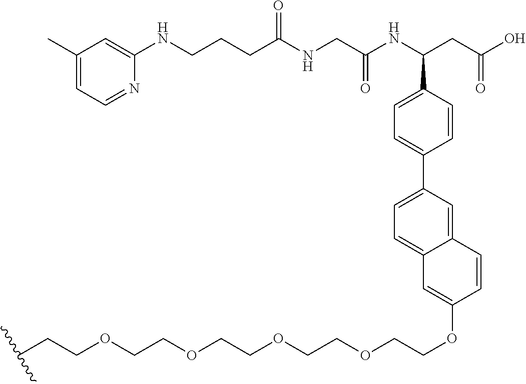 [SMILES: *CCOCCOCCOCCOCCOc1ccc2cc(-c3ccc([C@H](CC(=O)O)NC(=O)CNC(=O)CCCNc4cc(C)ccn4)cc3)ccc2c1] |
| Structure 19 | 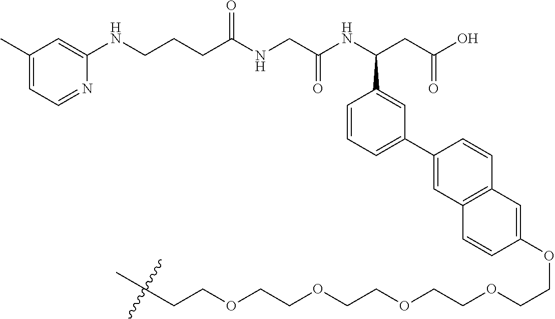 [SMILES: *CCOCCOCCOCCOCCOc1ccc2cc(-c3cccc([C@H](CC(=O)O)NC(=O)CNC(=O)CCCNc4cc(C)ccn4)c3)ccc2c1] |
| Structure 20 | 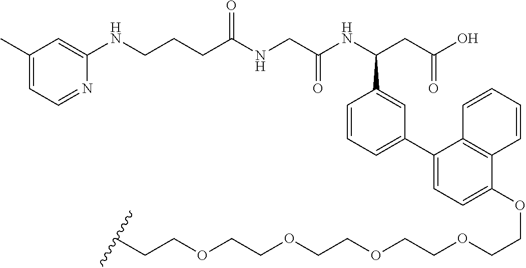 [SMILES: *CCOCCOCCOCCOCCOc1ccc(-c2cccc([C@H](CC(=O)O)NC(=O)CNC(=O)CCCNc3cc(C)ccn3)c2)c2ccccc12] |
| Structure 22 | 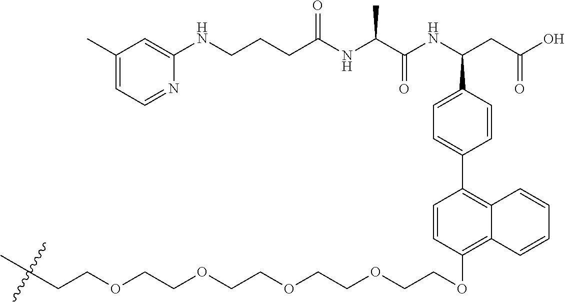 [SMILES: *CCOCCOCCOCCOCCOc1ccc(-c2ccc([C@H](CC(=O)O)NC(=O)[C@H](C)NC(=O)CCCNc3cc(C)ccn3)cc2)c2ccccc12] |
| Structure 23 | 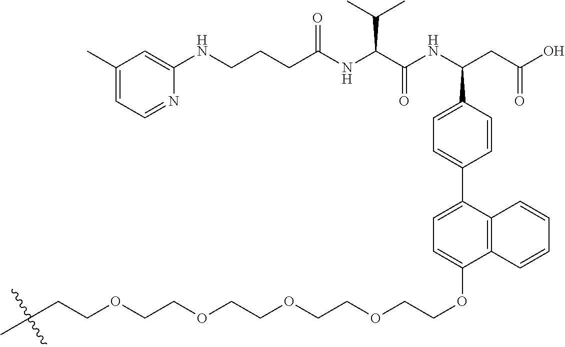 [SMILES: *CCOCCOCCOCCOCCOc1ccc(-c2ccc([C@H](CC(=O)O)NC(=O)[C@@H](NC(=O)CCCNc3cc(C)ccn3)C(C)C)cc2)c2ccccc12] |
| Structure 24 | 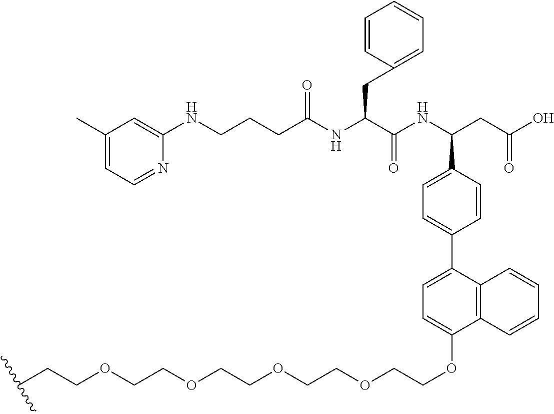 [SMILES: *CCOCCOCCOCCOCCOc1ccc(-c2ccc([C@H](CC(=O)O)NC(=O)[C@H](Cc3ccccc3)NC(=O)CCCNc3cc(C)ccn3)cc2)c2ccccc12] |
| Structure 25 | 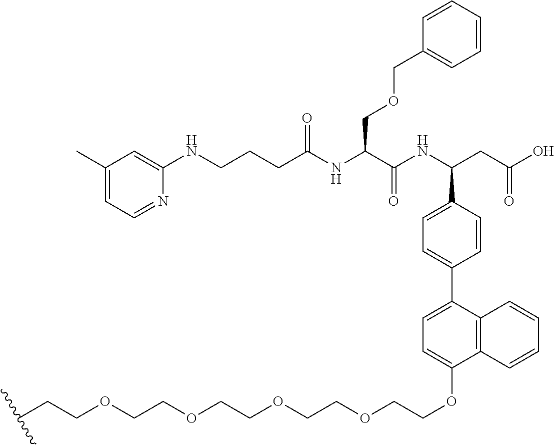 [SMILES: *CCOCCOCCOCCOCCOc1ccc(-c2ccc([C@H](CC(=O)O)NC(=O)[C@H](COCc3ccccc3)NC(=O)CCCNc3cc(C)ccn3)cc2)c2ccccc12] |
| Structure 27 | 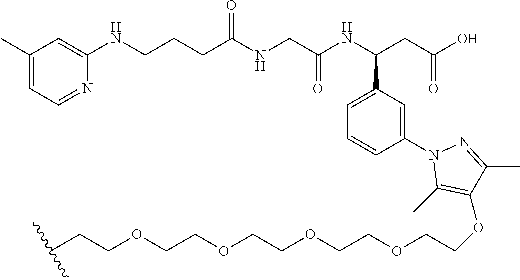 |
| Structure 29 | 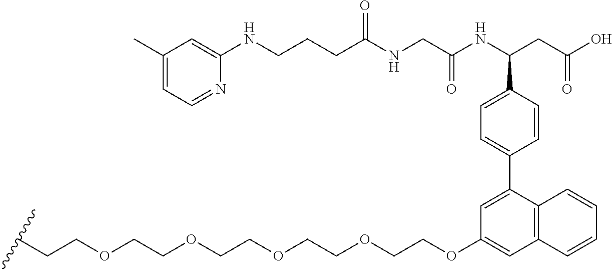 [SMILES: *CCOCCOCCOCCOCCOc1cc(-c2ccc([C@H](CC(=O)O)NC(=O)CNC(=O)CCCNc3cc(C)ccn3)cc2)c2ccccc2c1] |
| Structure 30 | 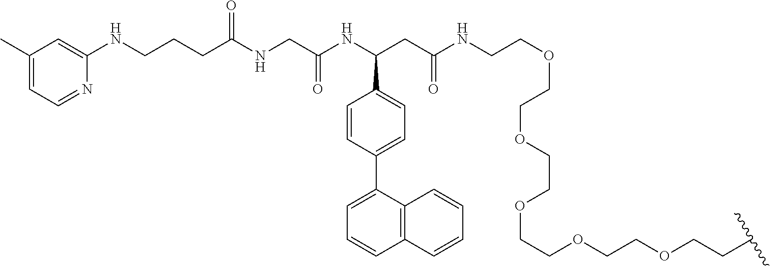 [SMILES: *CCOCCOCCOCCOCCOCCNC(=O)C[C@H](NC(=O)CNC(=O)CCCNc1cc(C)ccn1)c1ccc(-c2cccc3ccccc23)cc1] |
| Structure 31 | 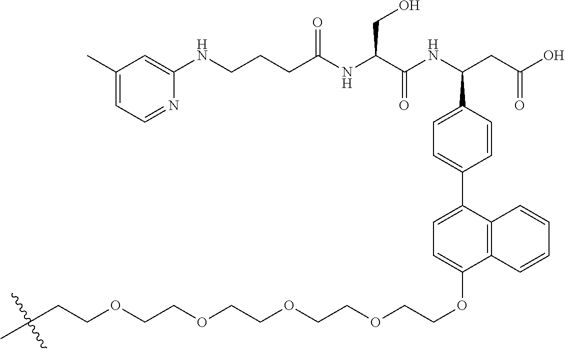 [SMILES: *CCOCCOCCOCCOCCOc1ccc(-c2ccc([C@H](CC(=O)O)NC(=O)[C@H](CO)NC(=O)CCCNc3cc(C)ccn3)cc2)c2ccccc12] |
| Structure 32 | 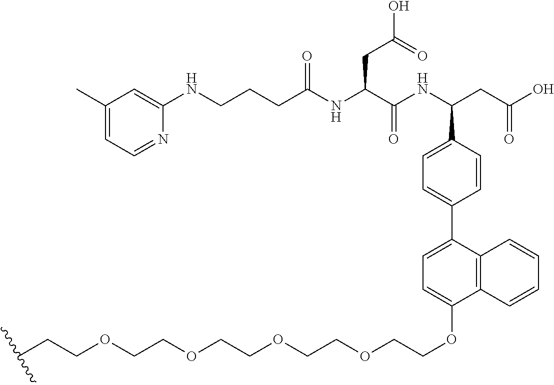 [SMILES: *CCOCCOCCOCCOCCOc1ccc(-c2ccc([C@H](CC(=O)O)NC(=O)[C@H](CC(=O)O)NC(=O)CCCNc3cc(C)ccn3)cc2)c2ccccc12] |
| Structure 33 | 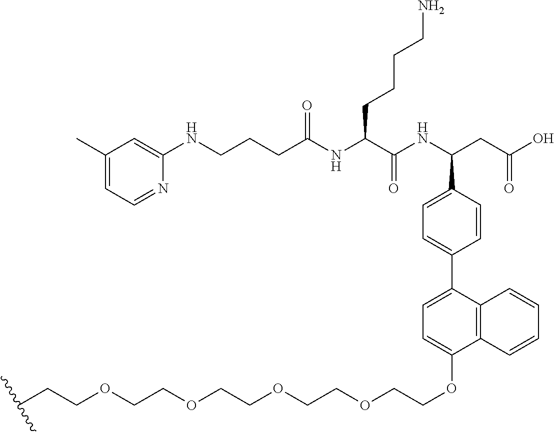 [SMILES: *CCOCCOCCOCCOCCOc1ccc(-c2ccc([C@H](CC(=O)O)NC(=O)[C@H](CCCCN)NC(=O)CCCNc3cc(C)ccn3)cc2)c2ccccc12] |
| Structure 34 | 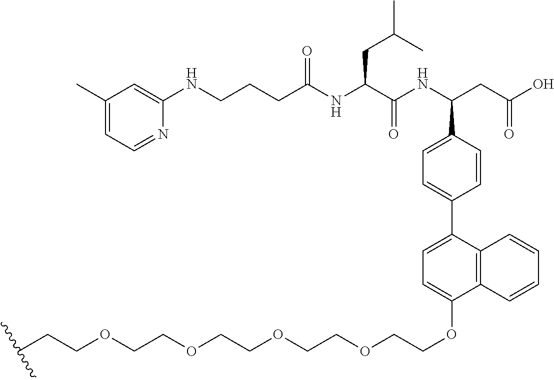 [SMILES: *CCOCCOCCOCCOCCOc1ccc(-c2ccc([C@H](CC(=O)O)NC(=O)[C@H](CC(C)C)NC(=O)CCCNc3cc(C)ccn3)cc2)c2ccccc12] |
| Structure 35 | 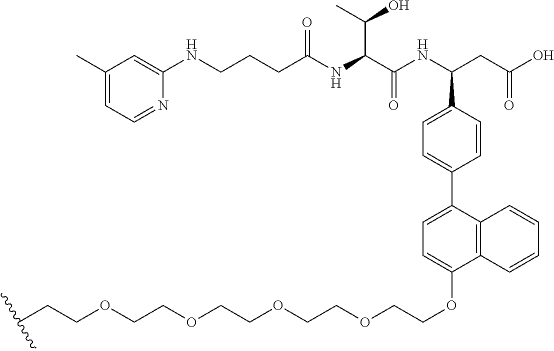 [SMILES: *CCOCCOCCOCCOCCOc1ccc(-c2ccc([C@H](CC(=O)O)NC(=O)[C@@H](NC(=O)CCCNc3cc(C)ccn3)[C@@H](C)O)cc2)c2ccccc12] |
| Structure 36 | 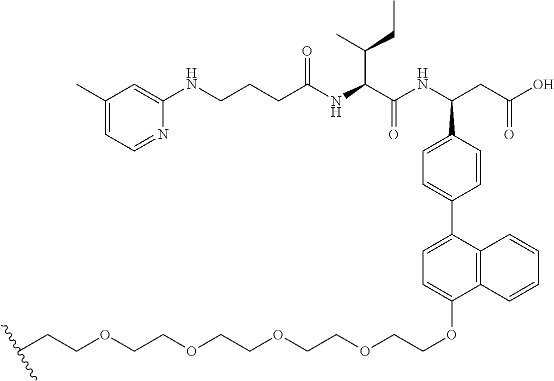 [SMILES: *CCOCCOCCOCCOCCOc1ccc(-c2ccc([C@H](CC(=O)O)NC(=O)[C@@H](NC(=O)CCCNc3cc(C)ccn3)[C@@H](C)CC)cc2)c2ccccc12] |
| Structure 37 | 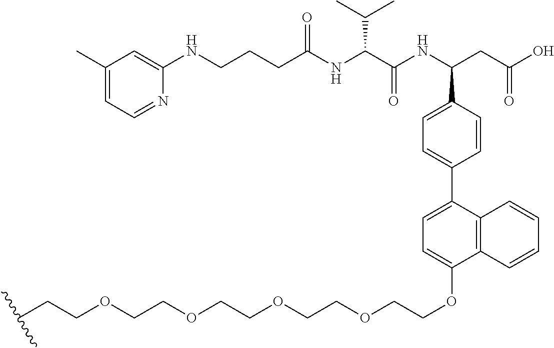 [SMILES: *CCOCCOCCOCCOCCOc1ccc(-c2ccc([C@H](CC(=O)O)NC(=O)[C@H](NC(=O)CCCNc3cc(C)ccn3)C(C)C)cc2)c2ccccc12] |

or a pharmaceutically acceptable salt thereof, wherein [symbol] indicates the point of connection to the RNAi agent.

In other embodiments, a delivery platform disclosed herein comprises one or more integrin targeting ligands that include a compound of the formula:

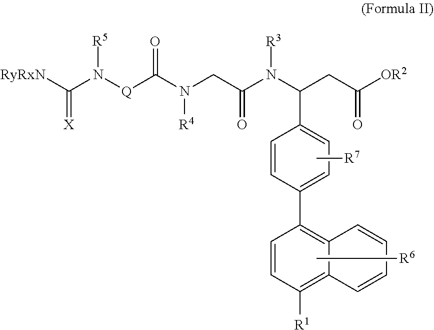

or a pharmaceutically acceptable salt thereof, wherein
R¹ is optionally substituted alkyl, optionally substituted alkoxy, or

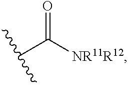

wherein R¹¹ and R¹² are each independently optionally substituted alkyl;
R² is H or optionally substituted alkyl;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino;
Q is optionally substituted aryl or optionally substituted alkylene;
X is O, CR⁸R⁹, NR⁸;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a cargo molecule; and
wherein when Q is optionally substituted alkyl and the length of the optionally substituted alkyl chain represented by Q is 3 carbons, then R¹ is

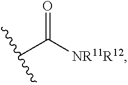

In some embodiments, compounds that may be conjugated to RNAi agents to synthesize a delivery platform for an RNAi agent are shown in Table 3 below:

TABLE 3
Compounds that may be conjugated to RNAi agents to synthesize a delivery platform for an RNAi agent.

| Compound | |
|---|---|
| Formula | Number |
| 40p | 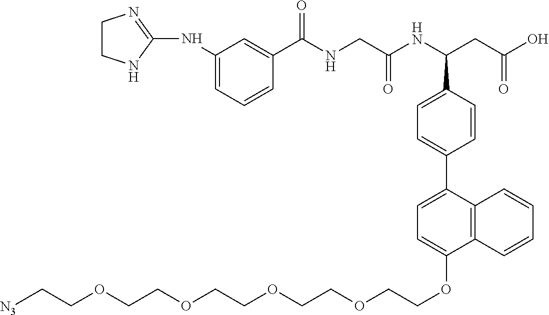 [SMILES: [N-]=[N+]=NCCOCCOCCOCCOCCOc1ccc(-c2ccc([C@H](CC(=O)O)NC(=O)CNC(=O)c3cccc(NC4=NCCN4)c3)cc2)c2ccccc12] |
| 41p | 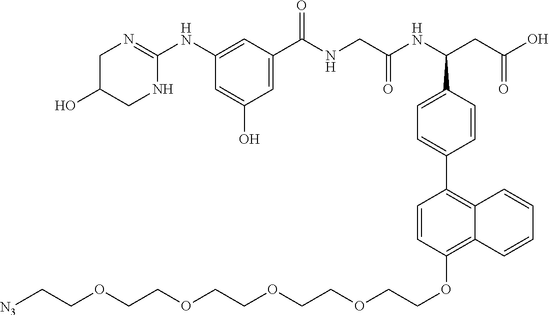 [SMILES: [N-]=[N+]=NCCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42p | 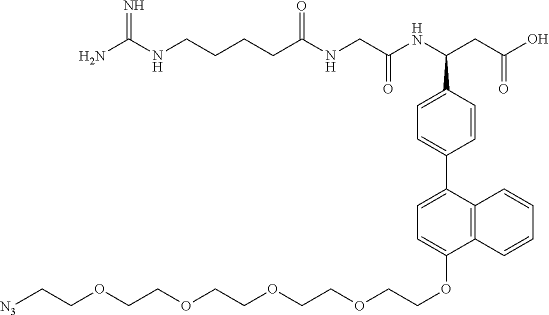 [SMILES: [N-]=[N+]=NCCOCCOCCOCCOCCOc1ccc(-c2ccc([C@H](CC(=O)O)NC(=O)CNC(=O)CCCCNC(=N)N)cc2)c2ccccc12] |
| 43p | 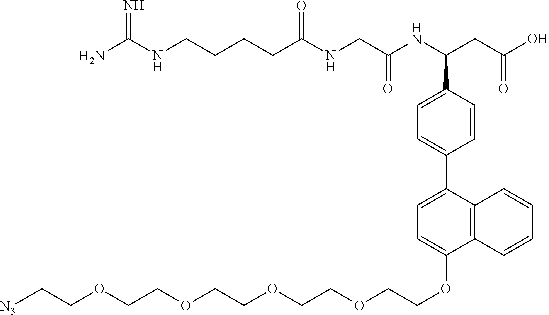 [SMILES: [N-]=[N+]=NCCOCCOCCOCCOCCOc1ccc(-c2ccc([C@H](CC(=O)O)NC(=O)CNC(=O)CCCCNC(=N)N)cc2)c2ccccc12] |
| 44p | 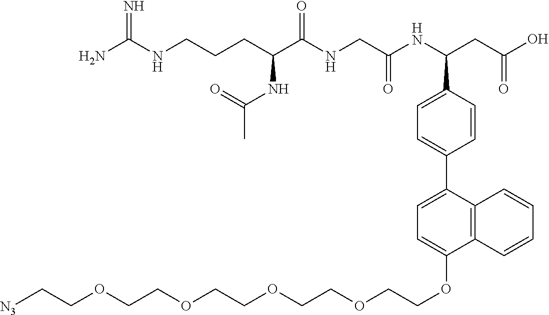 [SMILES: CC(=O)N[C@@H](CCCNC(=N)N)C(=O)NCC(=O)N[C@@H](CC(=O)O)c1ccc(-c2ccc(OCCOCCOCCOCCOCCN=[N+]=[N-])c3ccccc23)cc1] |
| 45p | 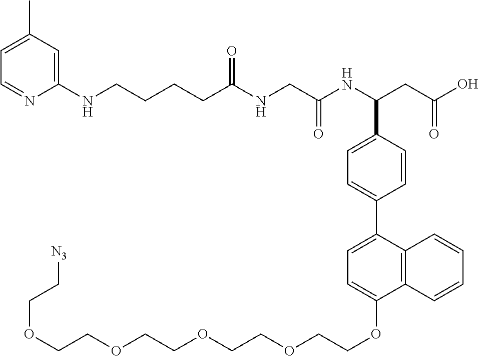 [SMILES: Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1] |
| 46p | 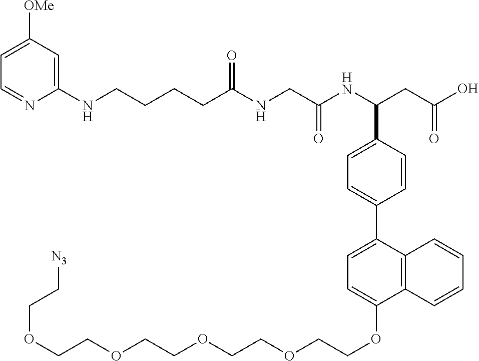 [SMILES: COc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1] |
| 47p | 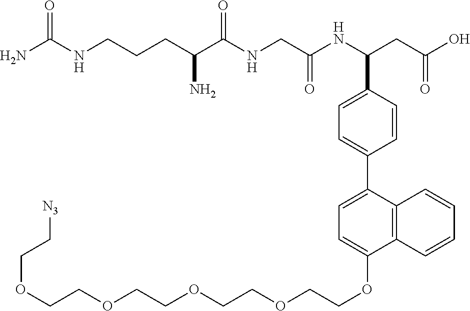 [SMILES: [N-]=[N+]=NCCOCCOCCOCCOCCOc1ccc(-c2ccc([C@H](CC(=O)O)NC(=O)CNC(=O)[C@@H](N)CCCNC(N)=O)cc2)c2ccccc12] |
| 48p | 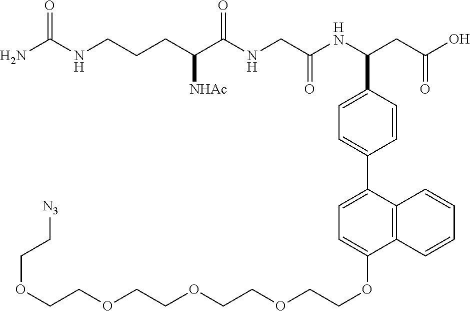 [SMILES: CC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1] |
| 49p | 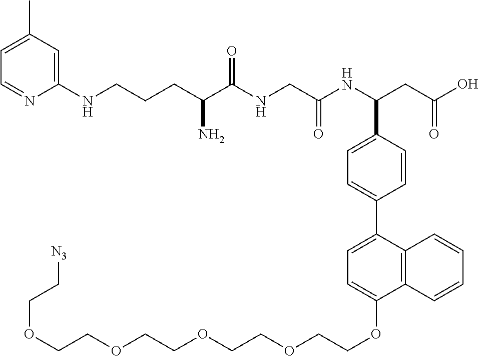 [SMILES: Cc1ccnc(NCCC[C@H](N)C(=O)NCC(=O)N[C@@H](CC(=O)O)c2ccc(-c3ccc(OCCOCCOCCOCCOCCN=[N+]=[N-])c4ccccc34)cc2)c1] |
| 50p | 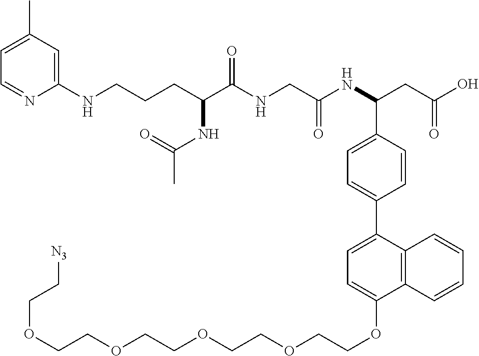 |
| 51p | 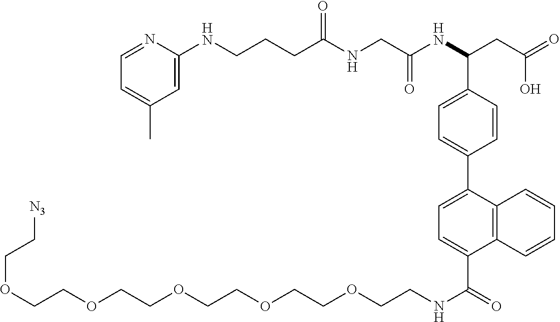 |
| 52p | 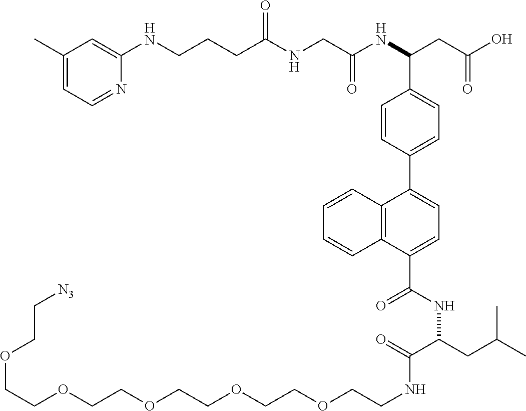 [SMILES: Cc1ccnc(NCCCC(=O)NCC(=O)N[C@@H](CC(=O)O)c2ccc(-c3ccc(C(=O)N[C@H](CC(C)C)C(=O)NCCOCCOCCOCCOCCOCCN=[N+]=[N-])c4ccccc34)cc2)c1] |
| 53p | 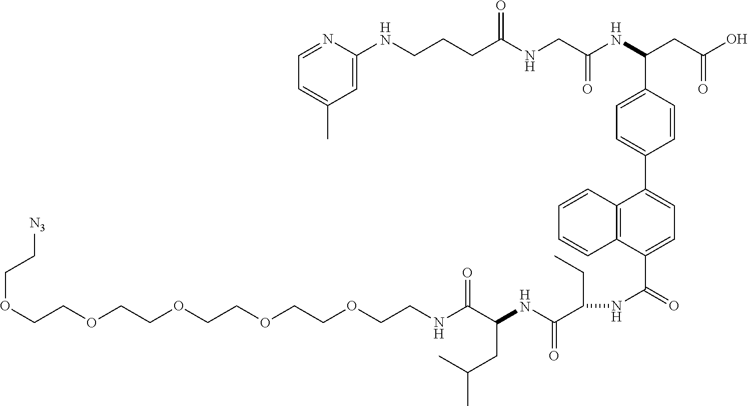 [SMILES: CC[C@H](NC(=O)c1ccc(-c2ccc([C@H](CC(=O)O)NC(=O)CNC(=O)CCCNc3cc(C)ccn3)cc2)c2ccccc12)C(=O)N[C@@H](CC(C)C)C(=O)NCCOCCOCCOCCOCCOCCN=[N+]=[N-]] |
| 54p | 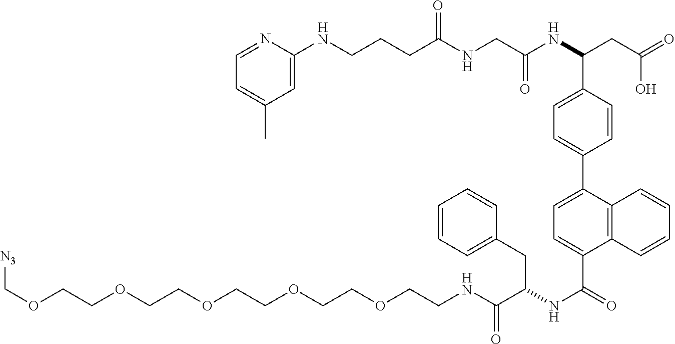 [SMILES: Cc1ccnc(NCCCC(=O)NCC(=O)N[C@@H](CC(=O)O)c2ccc(-c3ccc(C(=O)N[C@@H](Cc4ccccc4)C(=O)NCCOCCOCCOCCOCCOCN=[N+]=[N-])c4ccccc34)cc2)c1] |
| 55p | 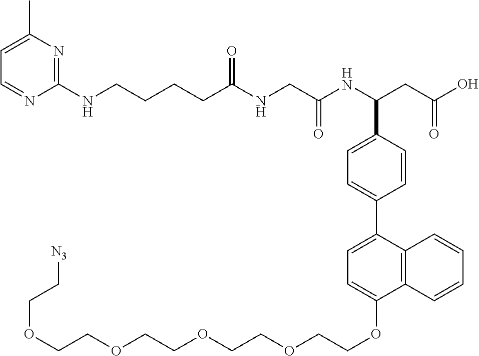 [SMILES: Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 56p | 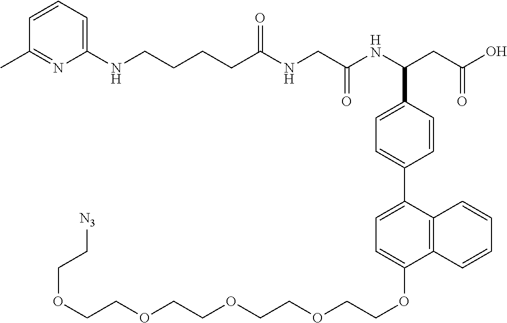 [SMILES: Cc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 57p | 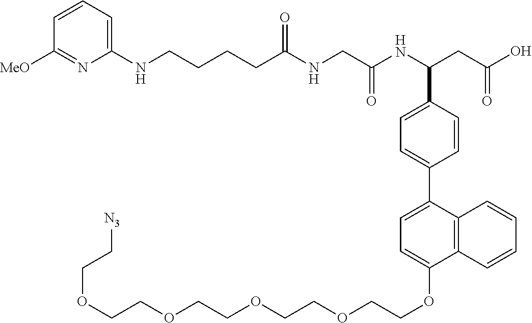 [SMILES: COc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1] |
| 58p | 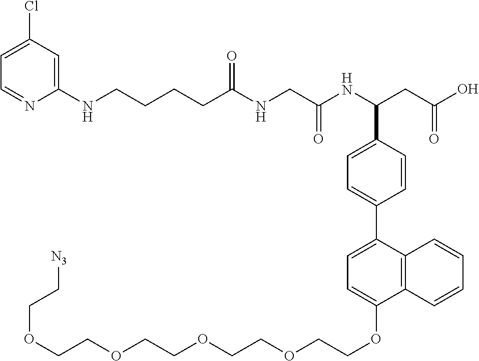 [SMILES: [N-]=[N+]=NCCOCCOCCOCCOCCOc1ccc(-c2ccc([C@H](CC(=O)O)NC(=O)CNC(=O)CCCCNc3cc(Cl)ccn3)cc2)c2ccccc12] |
| 59p | 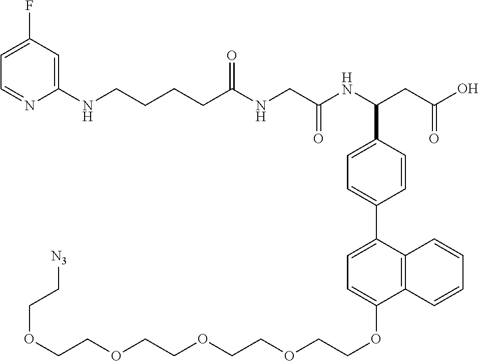 [SMILES: [N-]=[N+]=NCCOCCOCCOCCOCCOc1ccc(-c2ccc([C@H](CC(=O)O)NC(=O)CNC(=O)CCCCNc3cc(F)ccn3)cc2)c2ccccc12] |
| 60p | 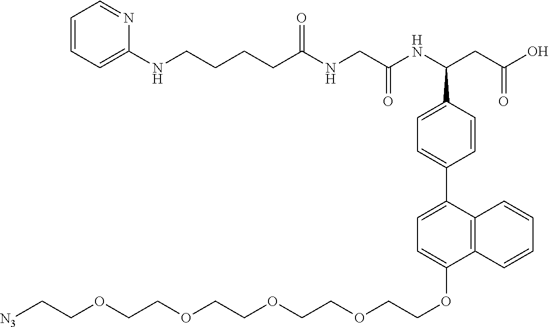 [SMILES: [N-]=[N+]=NCCOCCOCCOCCOCCOc1ccc(-c2ccc([C@H](CC(=O)O)NC(=O)CNC(=O)CCCCNc3ccccn3)cc2)c2ccccc12] |

In some embodiments, an RNAi agent may be linked to one or more integrin targeting ligands that include one or more of the structures in Table 4 below: Table 4. Integrin targeting ligands that may be linked to an RNAi agent.

TABLE 4
Integrin targeting ligands that may be linked to an RNAi agent.

| Compound | |
|---|---|
| Number | Formula |
| 40b | 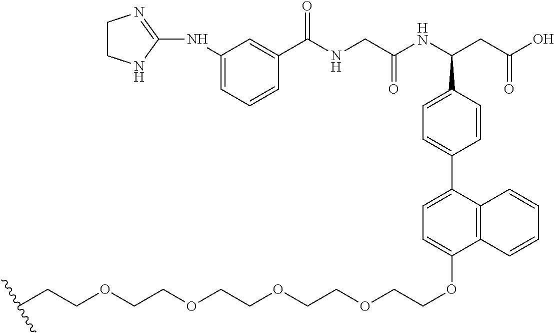 [SMILES: *CCOCCOCCOCCOCCOc1ccc(-c2ccc([C@H](CC(=O)O)NC(=O)CNC(=O)c3cccc(NC4=NCCN4)c3)cc2)c2ccccc12] |
| 41b | 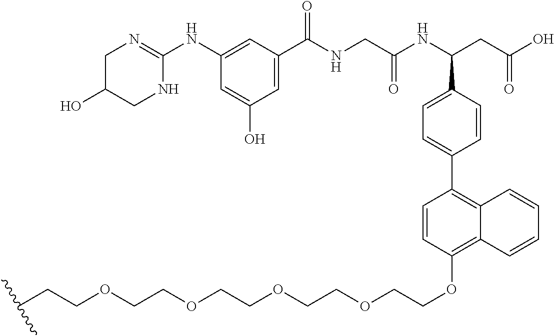 [SMILES: *CCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42b | 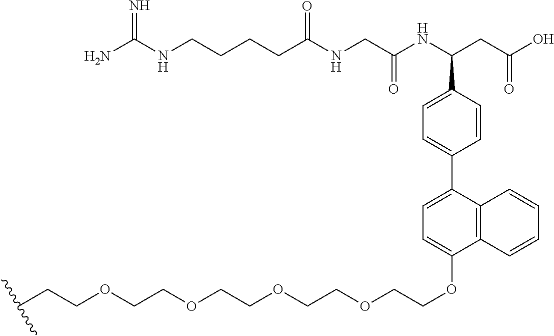 [SMILES: *CCOCCOCCOCCOCCOc1ccc(-c2ccc([C@H](CC(=O)O)NC(=O)CNC(=O)CCCCNC(=N)N)cc2)c2ccccc12] |
| 43b | 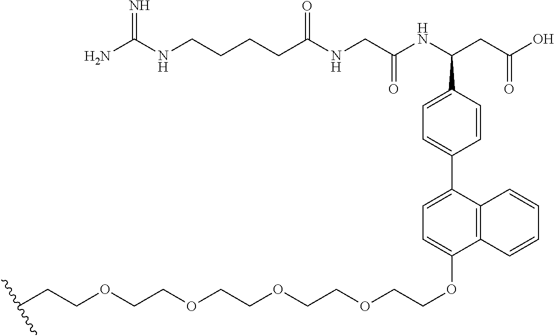 [SMILES: *CCOCCOCCOCCOCCOc1ccc(-c2ccc([C@H](CC(=O)O)NC(=O)CNC(=O)CCCCNC(=N)N)cc2)c2ccccc12] |
| 44b | 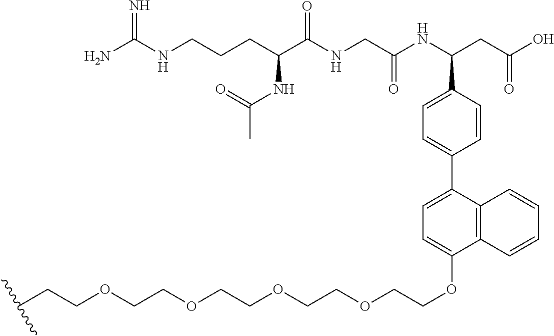 [SMILES: *CCOCCOCCOCCOCCOc1ccc(-c2ccc([C@H](CC(=O)O)NC(=O)CNC(=O)[C@H](CCCNC(=N)N)NC(C)=O)cc2)c2ccccc12] |
| 45b | 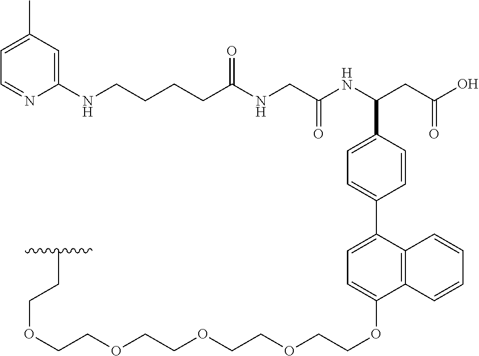 [SMILES: *CCOCCOCCOCCOCCOc1ccc(-c2ccc([C@H](CC(=O)O)NC(=O)CNC(=O)CCCCNc3cc(C)ccn3)cc2)c2ccccc12] |
| 46b | 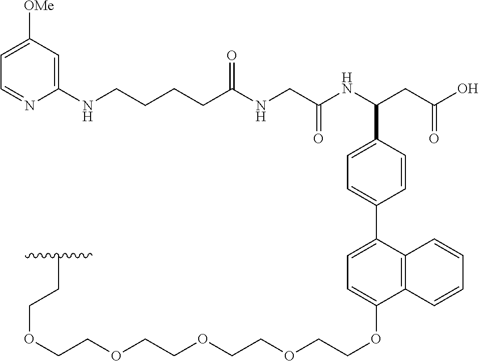 [SMILES: *CCOCCOCCOCCOCCOc1ccc(-c2ccc([C@H](CC(=O)O)NC(=O)CNC(=O)CCCCNc3cc(OC)ccn3)cc2)c2ccccc12] |
| 47b | 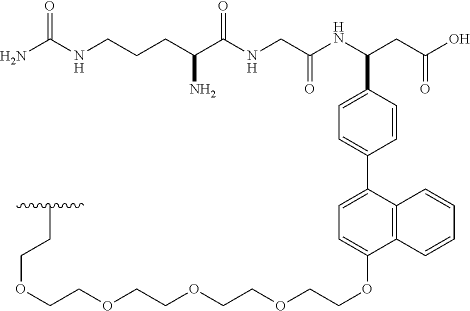 [SMILES: *CCOCCOCCOCCOCCOc1ccc(-c2ccc([C@H](CC(=O)O)NC(=O)CNC(=O)[C@@H](N)CCCNC(N)=O)cc2)c2ccccc12] |
| 48b | 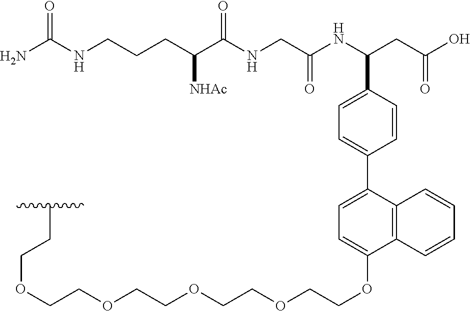 [SMILES: *CCOCCOCCOCCOCCOc1ccc(-c2ccc([C@H](CC(=O)O)NC(=O)CNC(=O)[C@H](CCCNC(N)=O)NC(C)=O)cc2)c2ccccc12] |
| 49b | 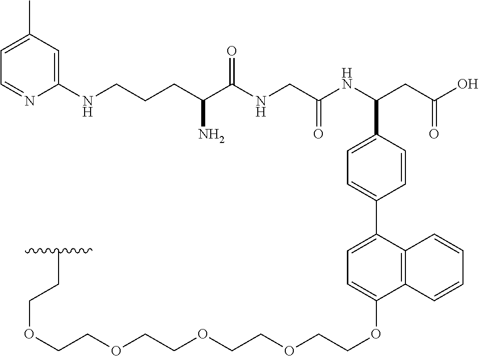 [SMILES: *CCOCCOCCOCCOCCOc1ccc(-c2ccc([C@H](CC(=O)O)NC(=O)CNC(=O)[C@@H](N)CCCNc3cc(C)ccn3)cc2)c2ccccc12] |
| 50b | 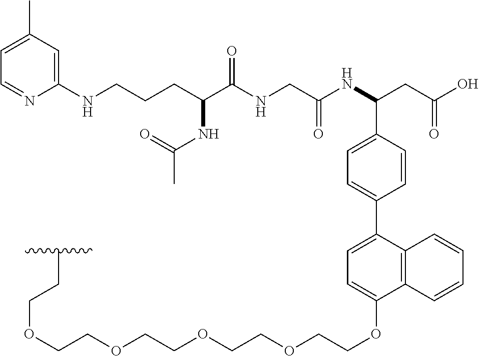 |
| 51b | 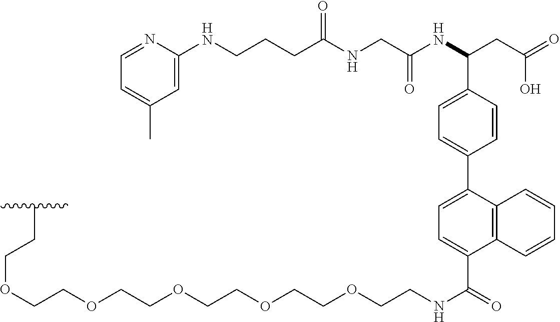 [SMILES: *CCOCCOCCOCCOCCOCCNC(=O)c1ccc(-c2ccc([C@H](CC(=O)O)NC(=O)CNC(=O)CCCNc3cc(C)ccn3)cc2)c2ccccc12] |
| 52b | 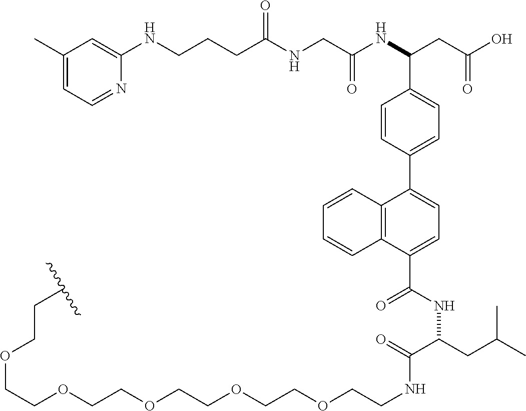 [SMILES: *CCOCCOCCOCCOCCOCCNC(=O)[C@@H](CC(C)C)NC(=O)c1ccc(-c2ccc([C@H](CC(=O)O)NC(=O)CNC(=O)CCCNc3cc(C)ccn3)cc2)c2ccccc12] |
| 53b | 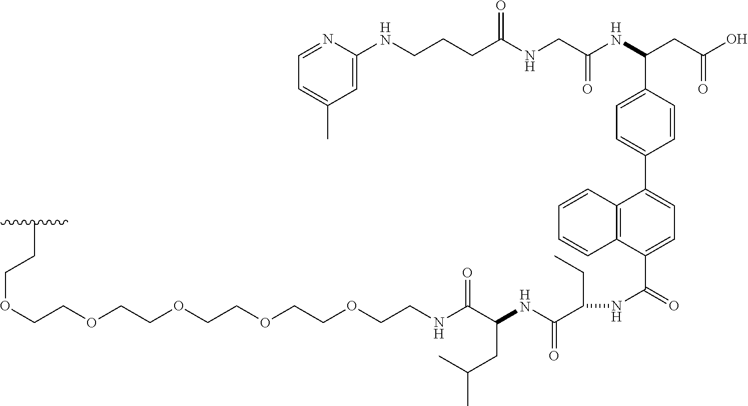 [SMILES: *CCOCCOCCOCCOCCOCCNC(=O)[C@H](CC(C)C)NC(=O)[C@H](CC)NC(=O)c1ccc(-c2ccc([C@H](CC(=O)O)NC(=O)CNC(=O)CCCNc3cc(C)ccn3)cc2)c2ccccc12] |
| 54b | 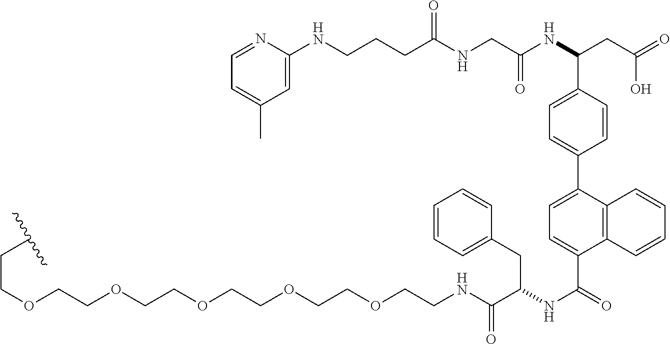 [SMILES: *CCOCCOCCOCCOCCOCCNC(=O)[C@H](Cc1ccccc1)NC(=O)c1ccc(-c2ccc([C@H](CC(=O)O)NC(=O)CNC(=O)CCCNc3cc(C)ccn3)cc2)c2ccccc12] |
| 55b | 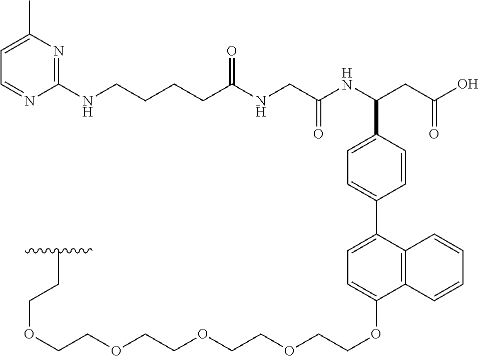 [SMILES: *CCOCCOCCOCCOCCOc1ccc(-c2ccc([C@H](CC(=O)O)NC(=O)CNC(=O)CCCCNc3nccc(C)n3)cc2)c2ccccc12] |
| 56b | 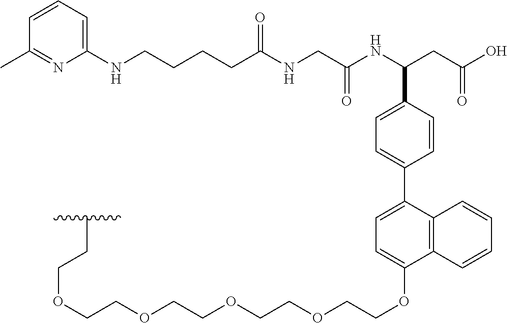 [SMILES: *CCOCCOCCOCCOCCOc1ccc(-c2ccc([C@H](CC(=O)O)NC(=O)CNC(=O)CCCCNc3cccc(C)n3)cc2)c2ccccc12] |
| 57b | 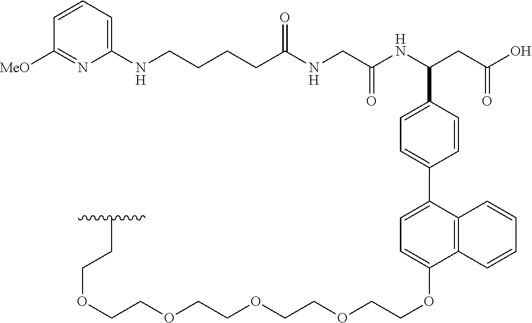 [SMILES: *CCOCCOCCOCCOCCOc1ccc(-c2ccc([C@H](CC(=O)O)NC(=O)CNC(=O)CCCCNc3cccc(OC)n3)cc2)c2ccccc12] |
| 58b | 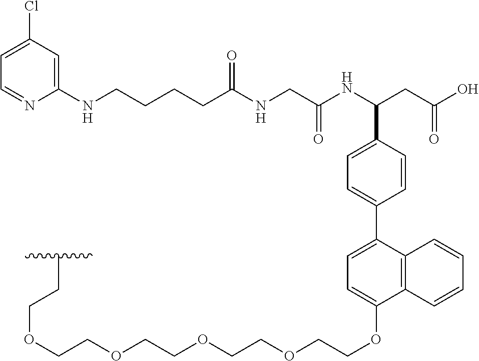 [SMILES: *CCOCCOCCOCCOCCOc1ccc(-c2ccc([C@H](CC(=O)O)NC(=O)CNC(=O)CCCCNc3cc(Cl)ccn3)cc2)c2ccccc12] |
| 59b | 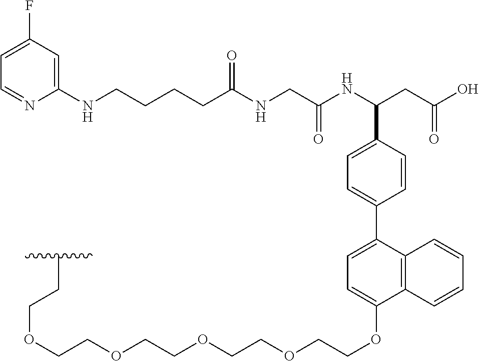 [SMILES: *CCOCCOCCOCCOCCOc1ccc(-c2ccc([C@H](CC(=O)O)NC(=O)CNC(=O)CCCCNc3cc(F)ccn3)cc2)c2ccccc12] |
| 60b | 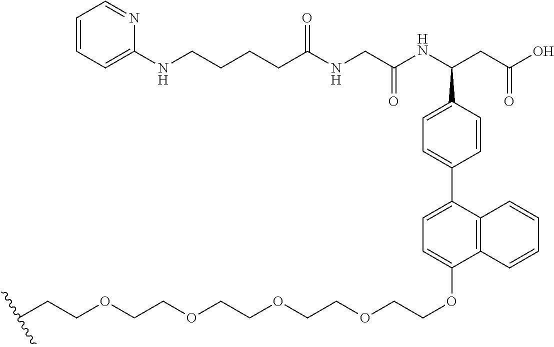 [SMILES: *CCOCCOCCOCCOCCOc1ccc(-c2ccc([C@H](CC(=O)O)NC(=O)CNC(=O)CCCCNc3ccccn3)cc2)c2ccccc12] |

wherein [symbol] indicates the point of connection to an RNAi agent.

In some embodiments, targeting groups are conjugated to an RNAi agent using a “click” chemistry reaction. In some embodiments, RNAi agents are functionalized with one or more alkyne-containing groups, and targeting ligands include azide-containing groups. Upon reaction, azides and alkynes form triazoles. An example reaction scheme is shown below:

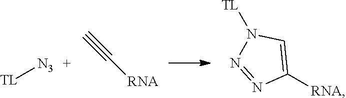

wherein TL comprises a targeting ligand, and RNA comprises an RNAi agent.

RNAi agents may comprise more than one targeting ligand. In some embodiments, RNAi agents comprise 1-20 targeting ligands. In some embodiments, RNAi agents comprise from 1, 2, 3, 4, 5, 6, 7, 8, 9, 10, 11, 12, 13, 14, 15, 16, 17, 18, or 19 targeting ligands to 2, 3, 4, 5, 6, 7, 8, 9, 10, 11, 12, 13, 14, 15, 16, 17, 18, 19 or 20 targeting ligands.

In some embodiments, RNAi agents comprise a targeting group, which includes 2 or more targeting ligands. In some embodiments, a targeting group may be conjugated at the 5′ or 3′ end of the sense strand of an RNAi agent. In some embodiments, a targeting group may be conjugated to an internal nucleotide on an RNAi agent. In some embodiments, a targeting group may consist of two targeting ligands linked together, referred to as a “bidentate” targeting group. In some embodiments, a targeting group may consist of three targeting ligands linked together, referred to as a “tridentate” targeting group. In some embodiments, a targeting group may consist of four targeting ligands linked together, referred to as a “tetradentate” targeting group.

In some embodiments, RNAi agents may comprise both a targeting group conjugated to the 3′ or 5′ end of the sense strand, and additionally targeting ligands conjugated to internal nucleotides. In some embodiments a tridentate targeting group is conjugated to the 5′ end of the sense strand of an RNAi agent, and at least one targeting ligand is conjugated to an internal nucleotide of the sense strand. In further embodiments, a tridentate targeting group is conjugated to the 5′ end of the sense strand of an RNAi agent, and four targeting ligands are conjugated to internal nucleotides of the sense strand.

Pharmacokinetic and/or Pharmacodynamic Modulators

Delivery vehicles disclosed herein comprise a pharmacokinetic and/or pharmacodynamic (also referred to herein as “PK/PD”) modulator linked to the RNAi agent to facilitate the delivery of the RNAi agent to the desired cells or tissues. PK/PD modulator precursors can be synthetized having reactive groups, such as maleimide or azido groups, to facilitate linkage to one or more linking groups on the RNAi agent. Chemical reaction syntheses to link such PK/PD modulator pecursors to RNAi agents are generally known in the art. The terms “PK/PD modulator” and “lipid PK/PD modulator” are used interchangeably herein.

In some embodiments, PK/PD modulators may include molecules that are fatty acids, lipids, albumin-binders, antibody-binders, polyesters, polyacrylates, poly-amino acids, and linear or branched polyethylene glycol (PEG) moieties having about 20-2000 PEG (CH₂—CH₂—O) units.

Table 5 shows certain exemplary PK/PD modulator precursors that can be used as starting materials to link to the RNAi agents disclosed herein. The PK/PD modulator precusors may be covalently attached to an RNAi agent using any known method in the art. In some embodiments, maleimide-containing PK/PD modulator precursors may be reacted with a disulfide-containing moiety at a 3′ end of the sense strand of the RNAi agent.

TABLE 5
Exemplary PK/PD Modulator Precursors Suitable for Linking to RNAi Agents. 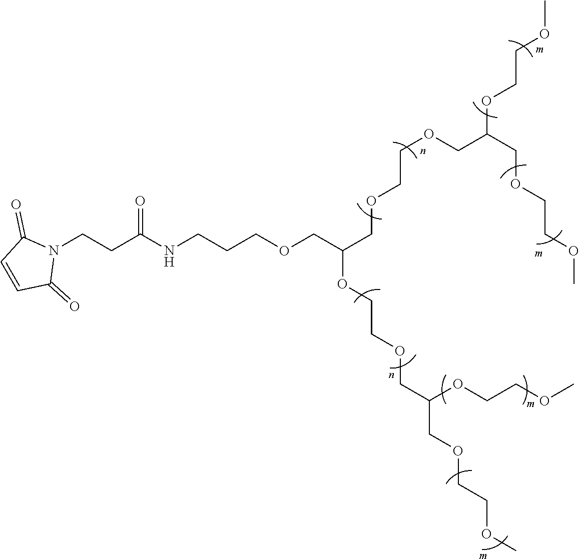
PEG40K (2x2-arm), wherein n and m are each independently integers, and the molecular weight of the sum of all PEG units is about 40 kilodaltons NOF, Sunbright ® GL4-400MA 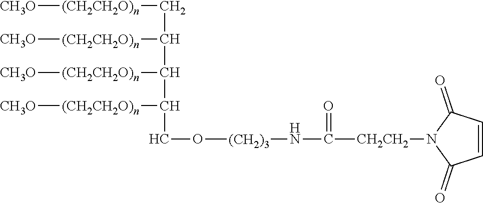
PEG40K (4-arm), wherein n is an integer, and the molecular weight of the sum of all PEG units is about 40 kilodaltons NOF, Sunbright ® XY4-400MA 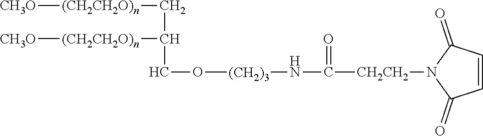
PEG40K (2-arm), wherein n is an integer, and the molecular weight of the sum of all PEG units is about 40 kilodaltons NOF, Sunbright ® GL2-400MA 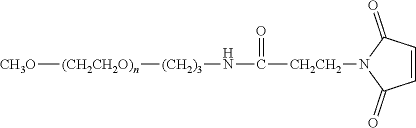
PEG40K, wherein n is an integer, and the molecular weight of the sum of all PEG units is about 40 kilodaltons NOF, Sunbright ® ME-400MA 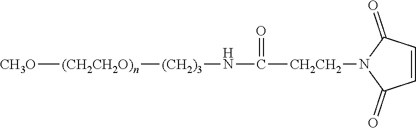
PEG10K, wherein n is an integer, and the molecular weight of the sum of all PEG units is about 10 kilodaltons NOF, Sunbright ® ME-100MA 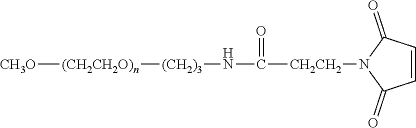
PEG5K, wherein n is an integer, and the molecular weight of the sum of all PEG units is about 5 kilodaltons NOF, Sunbright ® ME-050MA 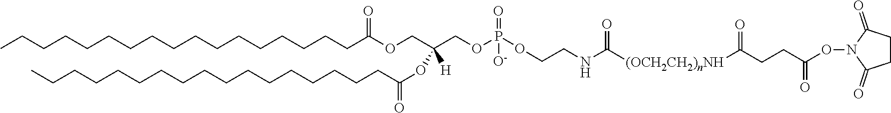
DSPE-PEG5K-NHS (Naonsoft Polymers ™ #SKU 1544) (1,2-distearoyl-sn-glycero-3-phosphoethanolamine-N-[succinimidyl(polyethylene glycol)]), wherein n is an integer, and the molecular weight of the sum of all PEG units is about 5 kilodaltons 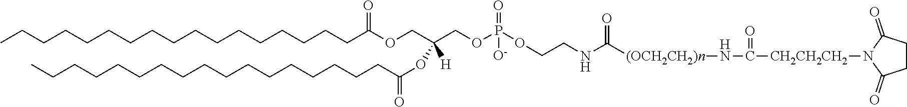
DSPE-PEG5K-MAL (Naonsoft Polymers ™ SKU #2049) 1,2-distearoyl-sn-glycero-3-phosphoethanolamine-N-[maleimide(polyethylene glycol)], Wherein n is an integer, and the molecular weight of the sum of all PEG units is about 5 kilodaltons 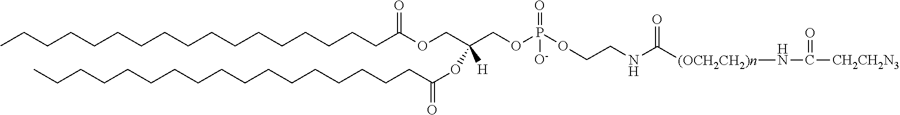
DSPE-PEG5K-N3 (Naonsoft Polymers ™ SKU #2274) 1,2-distearoyl-sn-glycero-3-phosphoethanolamine-N-[azido(polyethylene glycol)], wherein n is an integer, and the molecular weight of the sum of all PEG units is about 5 kilodaltons 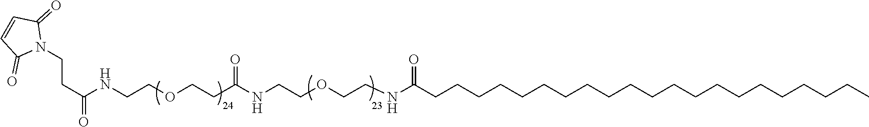
PEG47 + C22 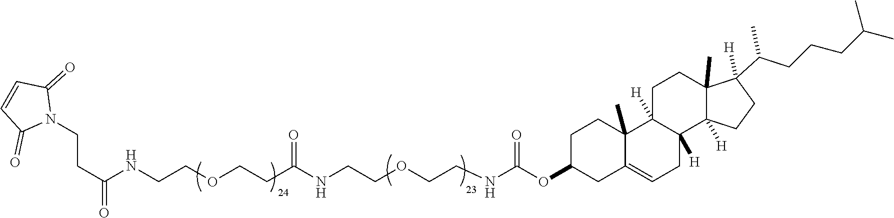
PEG47 + CLS (cholesterol) 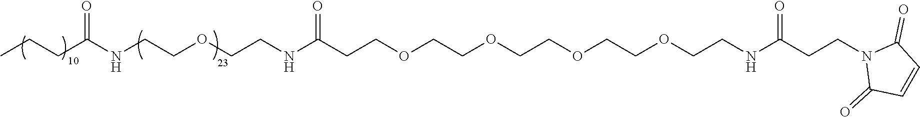
PEG23 + C22 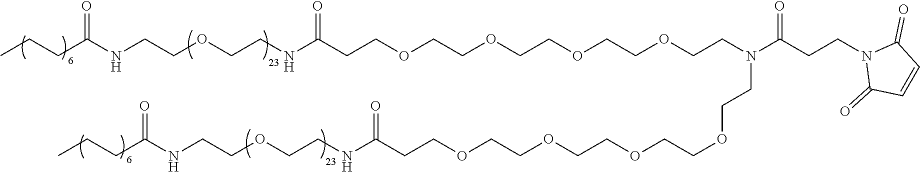
Bis(PEG23 + C14) 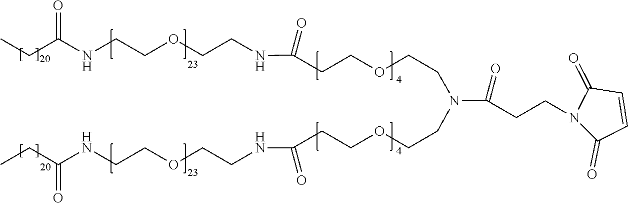
Bis(PEG23 + C22) 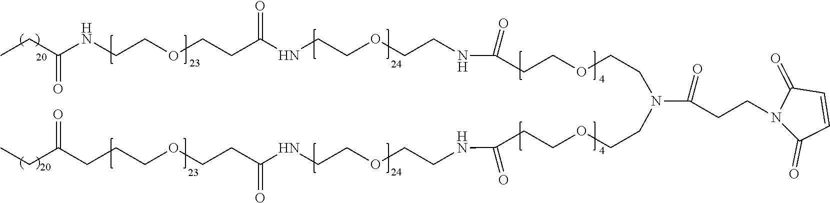
Bis(PEG47 + C22) 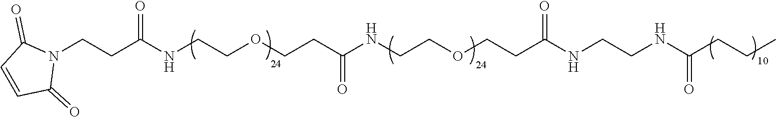
PEG48 + C22 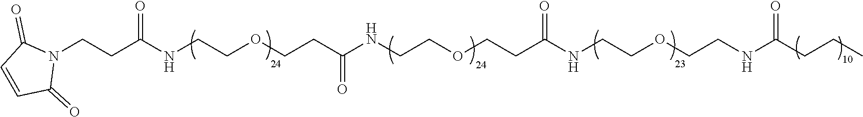
PEG71 + C22 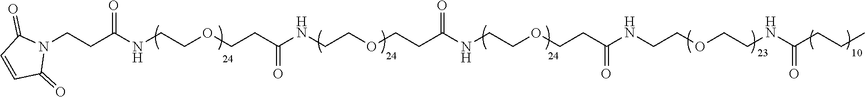
PEG95 + C22 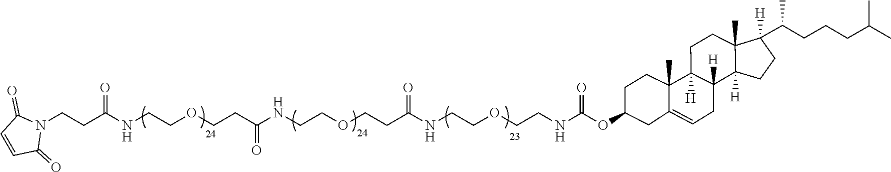
PEG71 + CLS 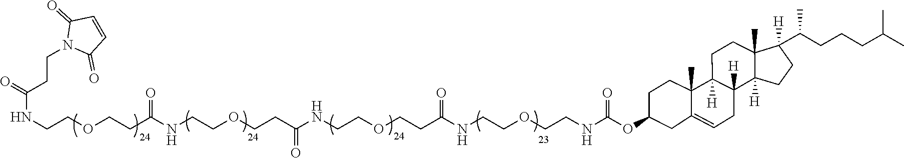
PEG95 + CLS 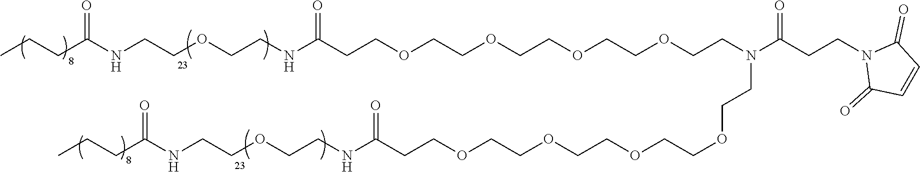
Bis(PEG23 + C18) 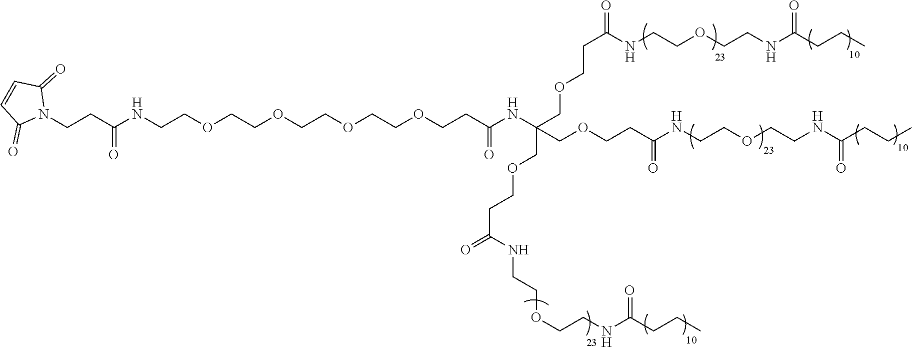
Tris(PEG23 + C22) 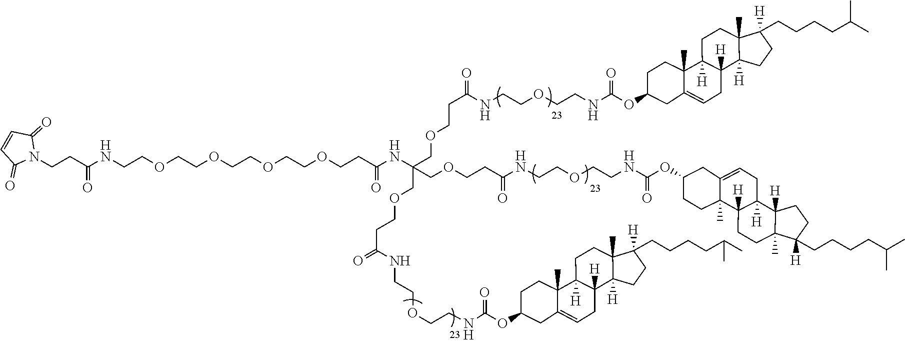
Tris(PEG23 + CLS) 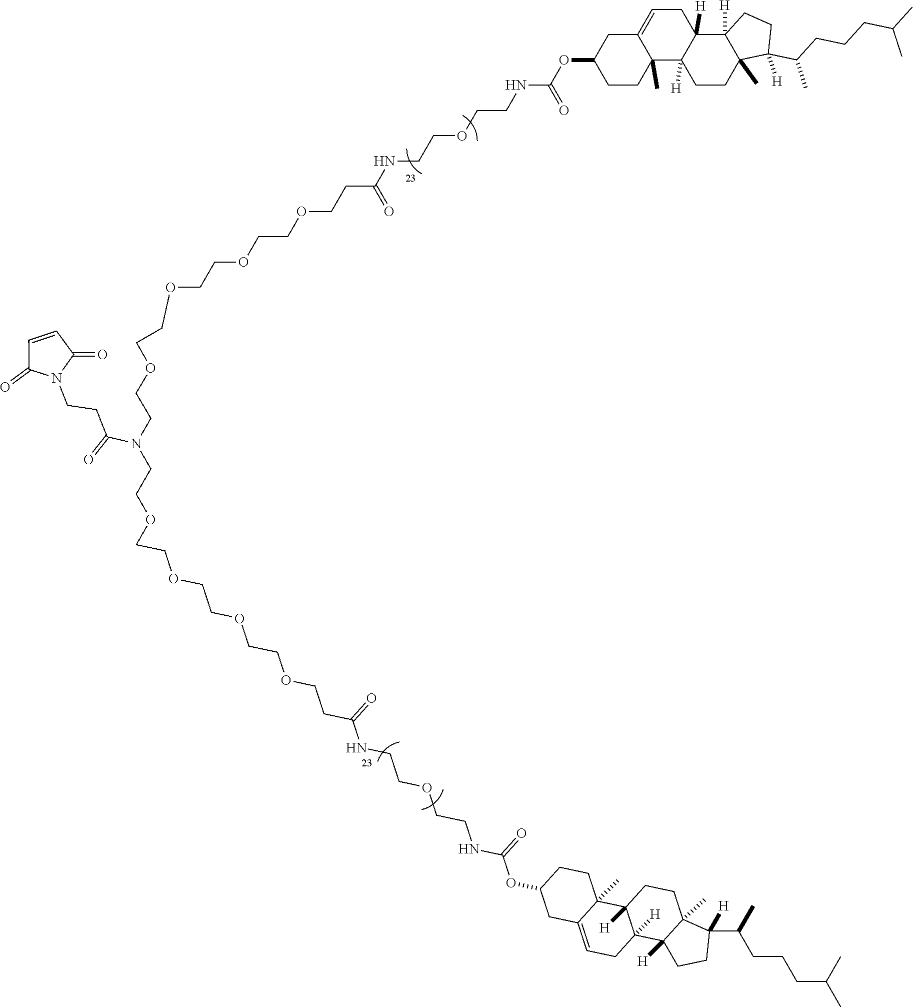
Bis(PEG23 + CLS) 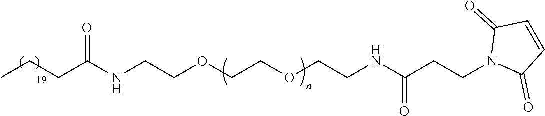
PEG5K + C22 wherein n is an integer, and the molecular weight of the sum of all PEG units is about 5 kilodaltons

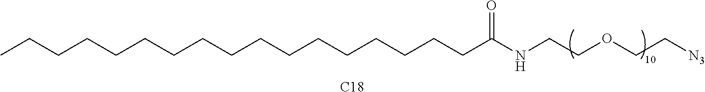

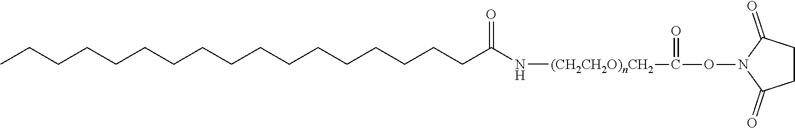

(NHS)-PEG1K + C18 (Naonsoft Polymers ™ SKU #10668-1000) wherein n is an integer, and the molecular weight of the sum of all PEG units is about 1 kilodalton 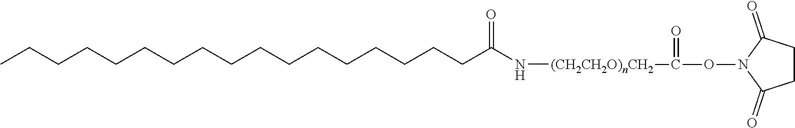
(NHS)-PEG2K + C18 (Naonsoft Polymers ™ SKU #10668-2000) wherein n is an integer, and the molecular weight of the sum of all PEG units is about 2 kilodaltons 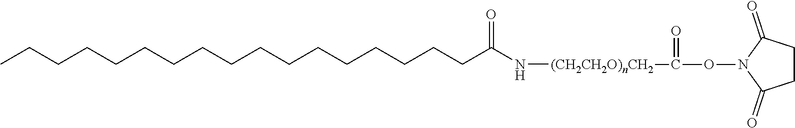
(NHS)-PEG5K + C18 (Naonsoft Polymers ™ SKU #10668-5000) wherein n is an integer, and the molecular weight of the sum of all PEG units is about 5 kilodaltons 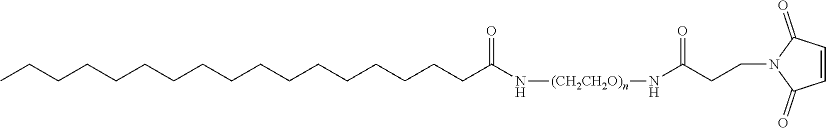
(MAL)-PEG5K + C18 (Naonsoft Polymers ™ SKU #10647) wherein n is an integer, and the molecular weight of the sum of all PEG units is about 5 kilodaltons 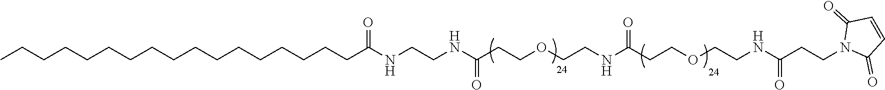
PEG48 + C18

In some embodiments, the RNAi agent may be conjugated to a lipid PK/PD modulator of Formula (I):

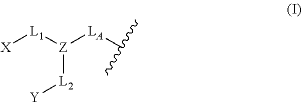

or a pharmaceutically acceptable salt thereof, wherein L_{A} is a bond or a bivalent moiety connecting Z to the RNAi agent; Z is CH, phenyl, or N; L₁ and L₂ are each independently linkers comprising at least about 5 polyethylene glycol (PEG) units; X and Y are each independently lipids comprising from about 10 to about 50 carbon atoms; and [symbol] indicates a point of connection to the RNAi agent.

In some embodiments, L₁ and L₂ each independently comprise about 15 to about 100 PEG units. In some embodiments, L₁ and L₂ each independently comprise about 20 to about 60 PEG units. In some embodiments, L₁ and L₂ each independently comprise about 20 to about 30 PEG units. In some embodiments, L₁ and L₂ each independently comprise about 40 to about 60 PEG units. In some embodiments, one of L₁ and L₂ comprises about 20 to about 30 peg units and the other comprises about 40 to about 60 PEG units. For example, L₁ and L₂ may each independently comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 PEG units. It will be appreciated that the PEG units of L₁ and L₂ need not be attached to form continuous chains, and that other moieties (e.g., a carbonyl moiety) may be incorporated to separate one set of PEG units from another set of PEG units.

In some embodiments, each of L₁ and L₂ is independently selected from the group consisting of the moieties identified in Table 6.

TABLE 6
Example L₁ and L₂ moieties of the present invention.

| Name | Structure |
|---|---|
| Linker 1 | 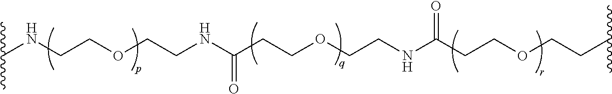 |
| Linker 2 | 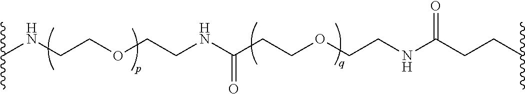 |
| Linker 3 | 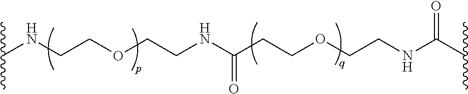 |
| Linker 4 | 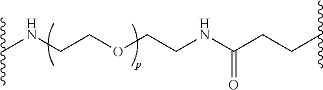 |
| Linker 5 | 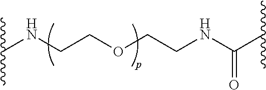 |
| Linker 6 | 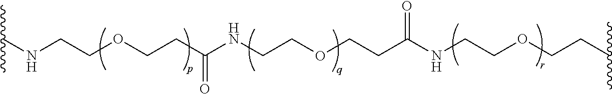 |
| Linker 7 | 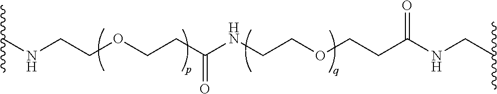 |
| Linker 8 | 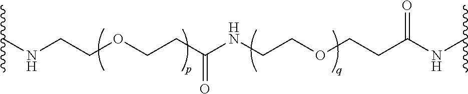 |
| Linker 9 | 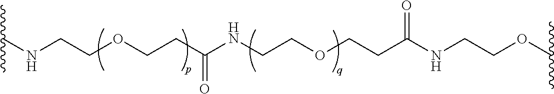 / |
| Linker 10 | 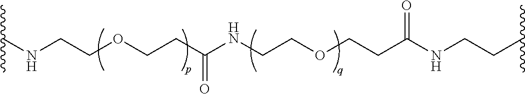 |
| Linker 11 | 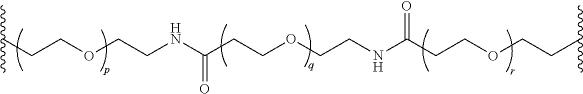 |
| Linker 12 | 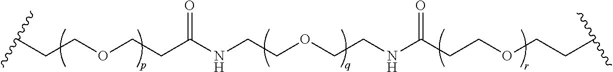 |

wherein, each p is independently 1, 2, 3, 4, 5, 6, 7, 8, 9, 10, 11, 12, 13, 14, 15, 17, 18, 19, 20, 21, 22, 23, 24, 25, 26, 27, 28, 29, or 30; each q is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each r is independently 1, 2, 3, 4, 5, 6, 7, 8, 9, or 10; and each [symbol] indicates a point of connection to X, Y, or Z, provided that: (i) in Linker 1, 6, and 11, p + q + r ≥ 5; (ii) in Linker 2, 3, 7, 8, 9, and 10, p + q ≥ 5; and (iii) in Linker 4 and p ≥ 5.

In some embodiments, each p is independently 20, 21, 22, 23, 24, or 25; each q is independently 20, 21, 22, 23, 24, or 25; and each r is independently 2, 3, 4, 5, or 6. In some embodiments, each p is independently 23 or 24. In some embodiments, each q is independently 23 or 24. In some embodiments, each r is 4.

In some embodiments, each of L₁ and L₂ is independently selected from the group consisting of the moieties identified in Table 7.

TABLE 7
Example L₁ and L₂ moieties of the present invention.
Structure

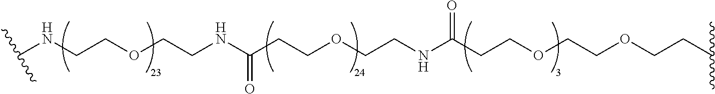

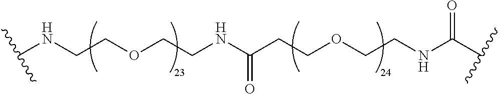

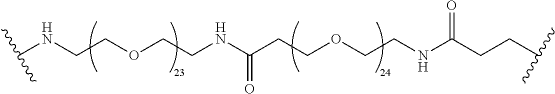

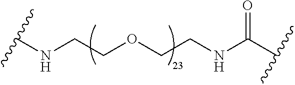

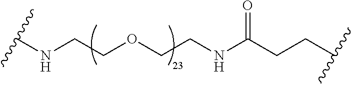

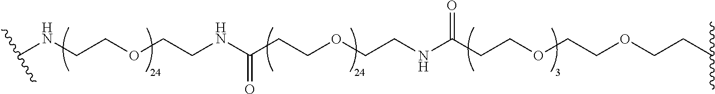

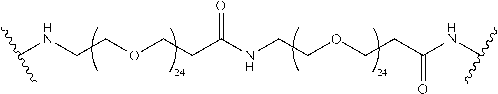

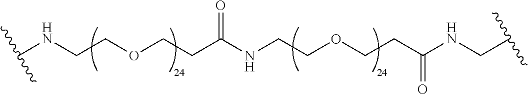

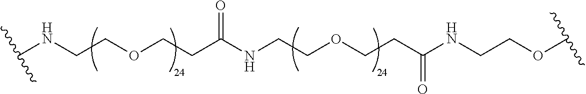

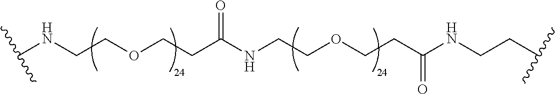

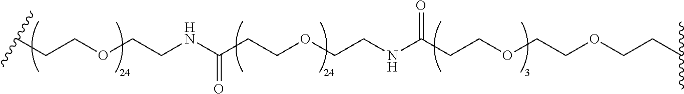

wherein [symbol] indicates a point of connection to X, Y, or Z.

In some embodiments, L₁ and L₂ are the same. In other embodiments, L₁ and L₂ are different.

In some embodiments, at least one of X and Y is an unsaturated lipid. In some embodiments, each of X and Y is an unsaturated lipid. In some embodiments, at least one of X and Y is a saturated lipid. In some embodiments, each of X and Y is a saturated lipid. In some embodiments, at least one of X and Y is a branched lipid. In some embodiments, each of X and Y is a branched lipid. In some embodiments, at least one of X and Y is a straight chain lipid. In some embodiments, each of X and Y is a straight chain lipid. In some embodiments, at least one of X and Y is cholesteryl. In some embodiments, each of X and Y is cholesteryl. In some embodiments, X and Y are the same. In other embodiments, X and Y are different.

In some embodiments, at least one of X and Y comprises from about 10 to about 45 carbon atoms. In some embodiments, at least one of X and Y comprises from about 10 to about 40 carbon atoms. In some embodiments, at least one of X and Y comprises from about 10 to about 35 carbon atoms. In some embodiments, at least one of X and Y comprises from about 10 to about 30 carbon atoms. In some embodiments, at least one of X comprises from about 10 to about 25 carbon atoms. In some embodiments, at least one of X and Y comprises from about 10 to about 20 carbon atoms.

In some embodiments, X and Y each independently comprise from about 10 to about 45 carbon atoms. In some embodiments, X and Y each independently comprise from about 10 to about 40 carbon atoms. In some embodiments, X and Y each independently comprise from about 10 to about 35 carbon atoms. In some embodiments, X and Y each independently comprise from about 10 to about 30 carbon atoms. In some embodiments, X and Y each independently comprise from about 10 to about 25 carbon atoms. In some embodiments, X and Y each independently comprise from about 10 to about 20 carbon atoms. For example, X and Y may each independently comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 carbon atoms.

In some embodiments, at least one of X and Y is selected from the group consisting of the moieties identified in Table 8. In some embodiments, each of X and Y are independently selected from the group consisting of the moieties identified in Table 8.

TABLE 8
Example X and Y moieties of the present invention.

| Name | Structure |
|---|---|
| Lipid 1 | 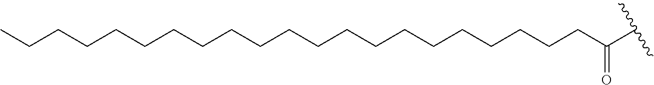 [SMILES: *C(=O)CCCCCCCCCCCCCCCCCCCCC] |
| Lipid 2 | 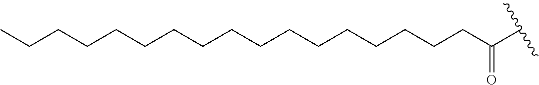 [SMILES: *C(=O)CCCCCCCCCCCCCCCCC] |
| Lipid 3 | 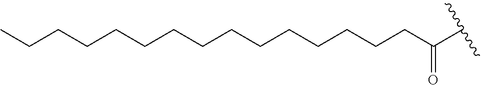 [SMILES: *C(=O)CCCCCCCCCCCCCCC] |
| Lipid 4 (Cho- lesteryl) | 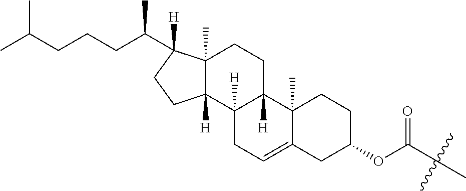 |
| Lipid 5 | 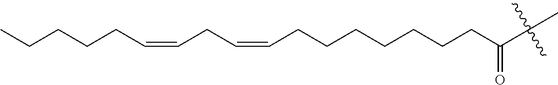 [SMILES: *C(=O)CCCCCCC/C=C\C/C=C\CCCCC] |
| Lipid 6 | 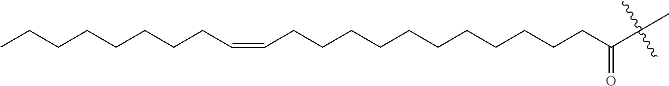 [SMILES: *C(=O)CCCCCCCCCCC/C=C\CCCCCCCC] |
| Lipid 7 | 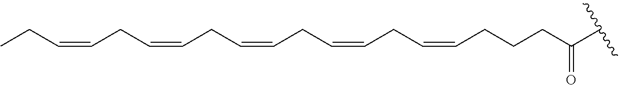 [SMILES: *C(=O)CCC/C=C\C/C=C\C/C=C\C/C=C\C/C=C\CC] |
| Lipid 8 | 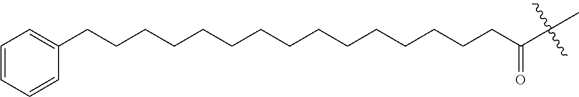 [SMILES: *C(=O)CCCCCCCCCCCCCCCc1ccccc1] |
| Lipid 9 | 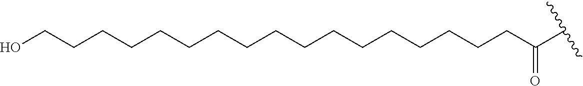 [SMILES: *C(=O)CCCCCCCCCCCCCCCCCO] |
| Lipid 10 | 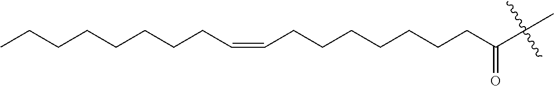 [SMILES: *C(=O)CCCCCCC/C=C\CCCCCCCC] |
| Lipid 11 | 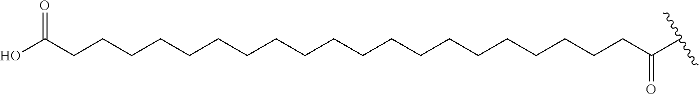 [SMILES: *C(=O)CCCCCCCCCCCCCCCCCCCCC(=O)O] |
| Lipid 12 | 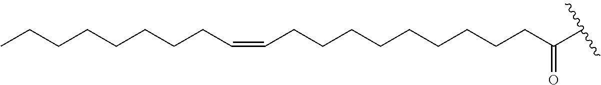 [SMILES: *C(=O)CCCCCCCCC/C=C\CCCCCCCC] |
| Lipid 14 | 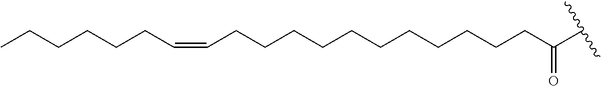 [SMILES: *C(=O)CCCCCCCCCCC/C=C\CCCCCC] |
| Lipid 15 | 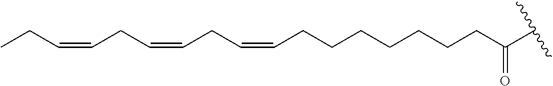 [SMILES: *C(=O)CCCCCCC/C=C\C/C=C\C/C=C\CC] |
| Lipid 16 | 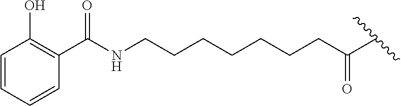 [SMILES: *C(=O)CCCCCCCNC(=O)c1ccccc1O] |
| Lipid 17 | 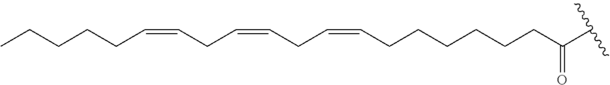 [SMILES: *C(=O)CCCCCC/C=C\C/C=C\C/C=C\CCCCC] |
| Lipid 18 | 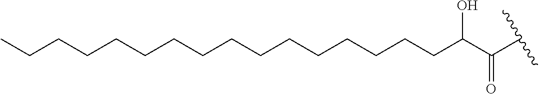 [SMILES: *C(=O)C(O)CCCCCCCCCCCCCCCC] |
| Lipid 19 | 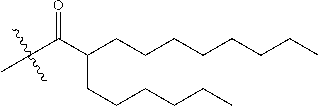 [SMILES: *C(=O)C(CCCCCC)CCCCCCCC] |
| Lipid 20 | 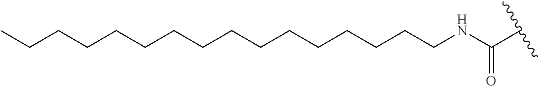 [SMILES: *C(=O)NCCCCCCCCCCCCCCCC] |
| Lipid 21 | 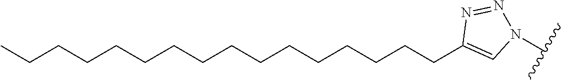 [SMILES: *n1cc(CCCCCCCCCCCCCCCC)nn1] |
| Lipid 22 | 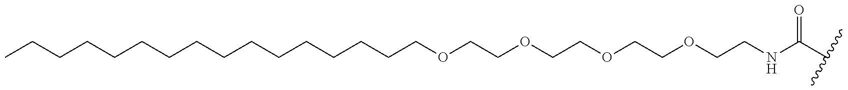 [SMILES: *C(=O)NCCOCCOCCOCCOCCCCCCCCCCCCCCCC] |
| Lipid 23 | 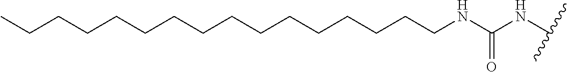 [SMILES: *NC(=O)NCCCCCCCCCCCCCCCC] |
| Lipid 24 | 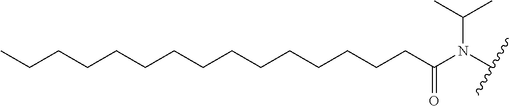 [SMILES: *N(C(=O)CCCCCCCCCCCCCCC)C(C)C] |

wherein [symbol] indicates a point of connection to L₁ or L₂.

In some embodiments, L_{A} comprises at least one PEG unit. In some embodiments, L_{A} is free of any PEG units. In some embodiments, L_{A} comprises —C(O)—, —C(O)NH—, optionally substituted alkoxy, or an optionally substituted alkyleneheterocyclyl. In some embodiments, L_{A} is a bond.

In some embodiments, L_{A} is selected from the group consisting of the moieties identified in Table 9.

TABLE 9
Example L_{A} moieties of the present invention.

| Name | Structure |
|---|---|
| Tether 1 | 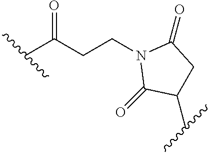 [SMILES: *C(=O)CCN1C(=O)CC(*)C1=O] |
| Tether 2 | 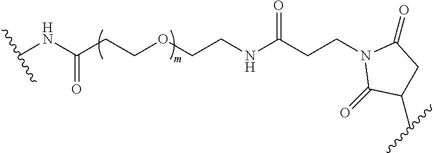 |
| Tether 3 | 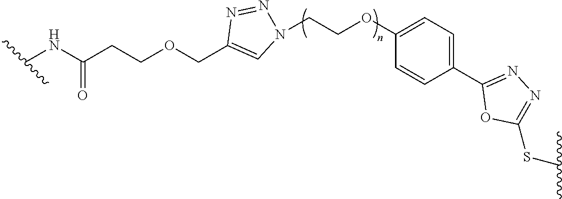 |
| Tether 4 | 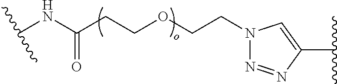 |
| Tether 5 | 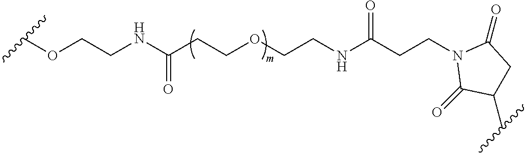 |
| Tether 6 | 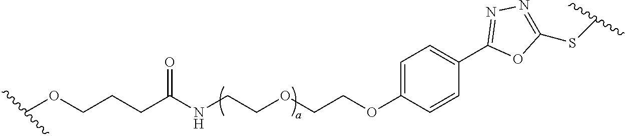 |
| Tether 7 | 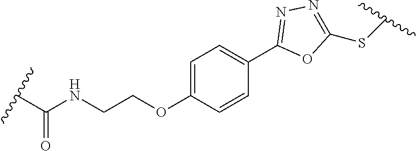 [SMILES: *Sc1nnc(-c2ccc(OCCNC(*)=O)cc2)o1] |
| Tether 8 | 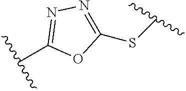 [SMILES: *Sc1nnc(*)o1] |
| Tether 9 | 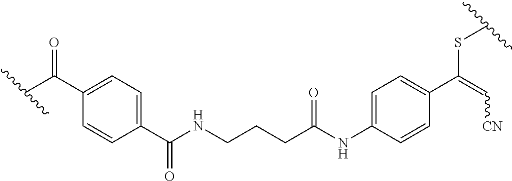 [SMILES: *SC(=CC#N)c1ccc(NC(=O)CCCNC(=O)c2ccc(C(*)=O)cc2)cc1] |
| Tether 10 | 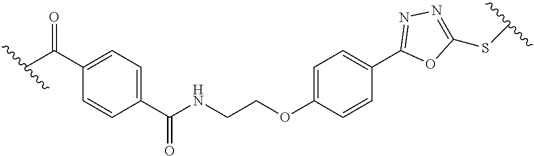 [SMILES: *Sc1nnc(-c2ccc(OCCNC(=O)c3ccc(C(*)=O)cc3)cc2)o1] |
| Tether 11 | 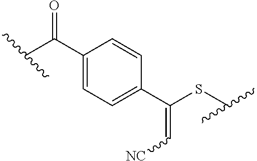 [SMILES: *SC(=CC#N)c1ccc(C(*)=O)cc1] |
| Tether 12 | 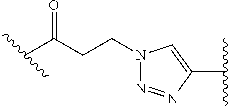 [SMILES: *C(=O)CCn1cc(*)nn1] |
| Tether 13 | 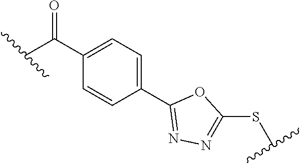 [SMILES: *Sc1nnc(-c2ccc(C(*)=O)cc2)o1] |
| Tether 14 | 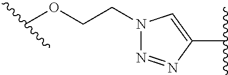 [SMILES: *OCCn1cc(*)nn1] |

wherein, each of m, n, o, and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to Z or the RNAi agent.

In some embodiments, each m is independently 1, 2, 3, 4, 5, 6, 7, 8, 9, 10, 20, 21, 22, 23, or 25; each n is independently 2, 3, 4, or 5; each a is independently 2, 3, or 4; and each o is independently 2, 3, 4, 5, 6, 7, 8, 9, 10, 11, 12, or 13. In some embodiments, each m is independently 2, 4, 8, or 24. In some embodiments, each n is 3. In some embodiments, each o is independently 4, 8, or 12. In some embodiments, each a is 3.

Another aspect of the present invention provides a lipid PK/PD modulator of Formula (Ia):

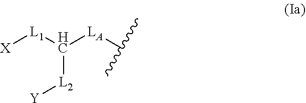

or a pharmaceutically acceptable salt thereof, wherein L_{A}, L₁, L₂, X, and Y are as defined in any of the embodiments of the lipid PK/PD modulator of Formula (I); and [symbol] indicates a point of connection to the RNAi agent.

In some embodiments, X and Y are each independently selected from the group consisting of Lipid 3, Lipid 4, Lipid, 5, Lipid 6, Lipid 7, Lipid 10, Lipid 12, and Lipid 19 as set forth in Table 8, wherein each [symbol] indicates a point of connection to L₁ or L₂.

In some embodiments, each of L₁ and L₂ is independently selected from the group consisting of Linker 2, Linker 3, Linker 4, and Linker 5 as set forth in Table 6, wherein each [symbol] indicates a point of connection to X, Y, or CH of Formula (Ia). In some embodiments, each p is 23. In some embodiments, each q is 24.

In some embodiments, L_{A} is selected from the group consisting of Tether 2, Tether 3, and Tether 4 as set forth in Table 5. In some embodiments, each m is independently 2, 4, 8, or 24. In some embodiments, each n is 4. In some embodiments, each o is independently 4, 8, or 12.

In some embodiments, L₁ and L₂ are independently selected from the group consisting of

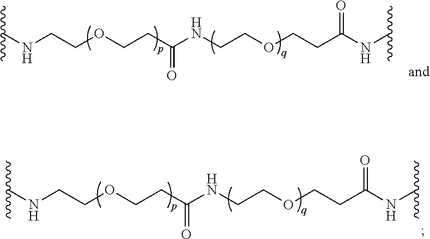

wherein, each p is independently 20, 21, 22, 23, 24, or 25; each q is independently 20, 21, 22, 23, 24, or 25; and each [symbol] indicates a point of connection to X, Y, or CH of Formula (Ia). In some embodiments, each p is 24. In some embodiments, each q is 24.

In some embodiments, L_{A} is

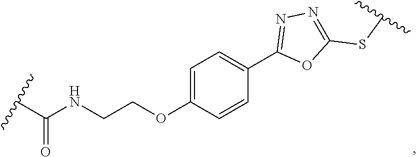 [SMILES: *Sc1nnc(-c2ccc(OCCNC(*)=O)cc2)o1]

and each [symbol] indicates a point of connection to the RNAi agent or CH of Formula (Ia).

In some embodiments, each of X and Y are

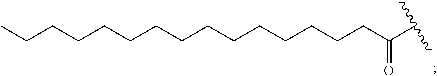 [SMILES: *C(=O)CCCCCCCCCCCCCCC]

wherein [symbol] indicates a point of connection to L₁ or L₂.

In some embodiments, the lipid PK/PD modulator of Formula (Ia) is selected from the group consisting of LP 210a or LP 217a as set forth in Table 19, or a pharmaceutically acceptable salt of any one of these lipid PK/PD modulators, wherein each L_{AA} is a bond or a bivalent moiety connecting the RNAi agent to the rest of the lipid PK/PD modulator, and each [symbol] indicates a point of connection to the RNAi agent.

In some embodiments, the lipid PK/PD modulator of Formula (Ia) is selected from the group consisting of LP 210b and LP 217b as set forth in Table 21, or a pharmaceutically acceptable salt of any one of these lipid PK/PD modulators, wherein each [symbol] indicates a point of connection to the RNAi agent.

Another aspect of the present invention provides a lipid PK/PD modulator of Formula (Ib):

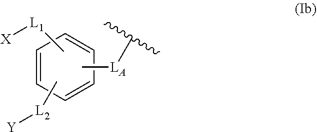

or a pharmaceutically acceptable salt thereof, wherein L_{A}, L₁, L₂, X, and Y are as defined in any of the embodiments of the lipid PK/PD modulator of Formula (I) or (Ia), and [symbol] indicates a point of connection to the RNAi agent.

In some embodiments, X and Y are each independently selected from the group consisting of Lipid 3 and Lipid 19 as set forth in Table 8, wherein each [symbol] indicates a point of connection to L₁ or L₂. In some embodiments, X and Y are each Lipid 3. In some embodiments, each of X and Y are each Lipid 19.

In some embodiments, each of L₁ and L₂ is independently selected from the group consisting of Linker 3, Linker 5, and Linker 9 as set forth in Table 6, wherein each [symbol] indicates a point of connection to X, Y, or the phenyl ring of Formula (Ib). In some embodiments, each p is 23 or 24. In some embodiments, each q is 24.

In some embodiments, L_{A} is selected from the group consisting of Tether 5, Tether, 6, Tether 7, Tether 8, and Tether 14 as set forth in Table 9, wherein each [symbol] indicates a point of connection to the RNAi agent or the phenyl ring of Formula (Ib). In some embodiments, each m is 2 or 4. In some embodiments, each a is 3.

Another aspect of the present invention provides lipid PK/PD modulator of Formula (Ib1):

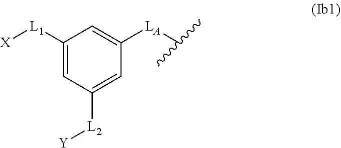

or a pharmaceutically acceptable salt thereof, wherein L_{A}, L₁, L₂, X, and Y are as defined in any of the embodiments of the lipid PK/PD modulator of Formula (I), (Ia), or (Ib), and [symbol] indicates a point of connection to the RNAi agent.

Another aspect of the present invention provides a lipid PK/PD modulator of Formula (Ic):

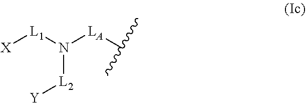

or a pharmaceutically acceptable salt thereof, wherein L_{A}, L₁, L₂, X, and Y are as defined in any of the embodiments of the lipid PK/PD modulator of Formula (I), (Ia), (Ib), or (Ib1), and [symbol] indicates a point of connection to the RNAi agent.

In some embodiments, X and Y are each independently selected from the group consisting of Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, and Lipid 24 as set forth in Table 4, wherein each [symbol] indicates a point of connection to L₁ and L₂. In some embodiments, each of X and Y is Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, or Lipid 24.

In some embodiments, each of L₁ and L₂ is independently selected from the group consisting of Linker 1, Linker 6, Linker 10, Linker 11, and Linker 12 as set forth in Table 2, wherein each [symbol] indicates a point of connection to X, Y, or N of Formula (Ic). In some embodiments, each p is independently 23 or 24. In some embodiments, each q is independently 23 or 24. In some embodiments, each r is 4.

In some embodiments, L_{A} is selected from the group consisting of Tether 1, Tether 9, Tether 10, Tether 11, Tether 12, and Tether 13 as set forth in Table 9, wherein each [symbol] indicates a point of connection to the RNAi agent or N of Formula (Ic).

Another aspect of the present invention provides a lipid PK/PD modulator of Formula (Id):

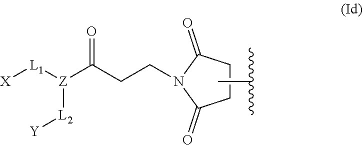

or a pharmaceutically acceptable salt thereof, wherein Z, L₁, L₂, X, and Y are as defined in any of the embodiments of the lipid PK/PD modulator of Formula (I), (Ia), (Ib) (Ib1), or (Ic), and [symbol] indicates a point of connection to the RNAi agent.

Another aspect of the present invention provides a lipid PK/PD modulator of Formula (II):

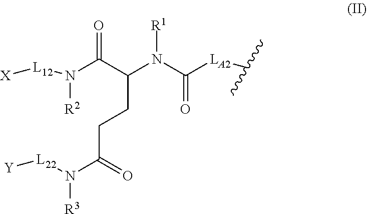

or a pharmaceutically acceptable salt thereof, wherein X and Y are as defined for any embodiments of the lipid PK/PD modulator of Formula (I), (Ia), (Ib), (Ib1), (Ic), or (Id); L₁₂ is L₁ as defined for any embodiments of the lipid PK/PD modulator of Formula (I), (Ia), (Ib), (Ib1), (Ic), or (Id); L₂₂ is L₂ as defined for any embodiments of the lipid PK/PD modulator of Formula (I), (Ia), (Ib), (Ib1), (Ic), or (Id); L_{A2} is L_{A} as defined for any embodiments of the lipid PK/PD modulator of Formula (I), (Ia), (Ib), (Ib1), or (Ic); R¹, R² and R³ are each independently hydrogen or C₁₋₆ alkyl; and [symbol] indicates a point of connection to the RNAi agent.

In some embodiments; L_{A2} is a bond or a bivalent moiety connecting the RNAi agent to —C(O)—; R¹, R² and R³ are each independently hydrogen or C₁₋₆ alkyl; L₁₂ and L₂₂ are each independently linkers comprising at least about 5 PEG units; X and Y are each independently lipids comprising from about 10 to about 50 carbon atoms; and [symbol] indicates a point of connection to the RNAi agent.

In some embodiments, each of L₁₂ and L₂₂ is independently selected from the group consisting of the moieties identified in Table 10.

TABLE 10
Example L₁₂ and L₂₂ moieties of the present invention.

| Name | Structure |
|---|---|
| Linker 1-2 | 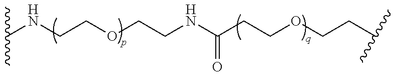 |
| Linker 2-2 | 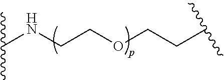 |

wherein, p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and each [symbol] indicates a point of connection to X, Y, —NR²—, or —NR³—, provided that: (i) in Linker 1-2, p + q ≥ 5; and (ii) in Liner 2-2, p ≥ 5.

In some embodiments, each p is independently 20, 21, 22, 23, 24, or 25. In some embodiments, each q is independently 20, 21, 22, 23, 24, or 25. In some embodiments, each p is independently 23 or 24. In some embodiments, each p is 23. In some embodiments, each q is 24.

In some embodiments, L₁₂ and L₂₂ are the same. In other embodiments, L₁₂ and L₂₂ are different.

In some embodiments, at least one of X and Y is selected from the group consisting of the moieties identified in Table 8, wherein each [symbol] indicates a point of connection to L₁₂ or L₂₂. In some embodiments, each of X and Y is independently selected from the group consisting of the moieties identified in Table 8, wherein each [symbol] indicates a point of connection to L₁₂ or L₂₂.

In some embodiments, at least one of X and Y is selected from the group consisting of the moieties identified in Table 11. In some embodiments, each of X and Y is independently selected from the group consisting of the moieties identified in Table 11.

TABLE 11
Example X and Y moieties of the lipid PK/PD modulator of Formula (II).

| Name | Structure |
|---|---|
| Lipid 3 | 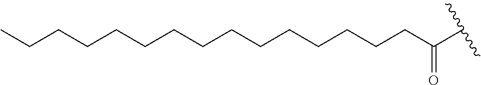 [SMILES: *C(=O)CCCCCCCCCCCCCCC] |
| Lipid 4 | 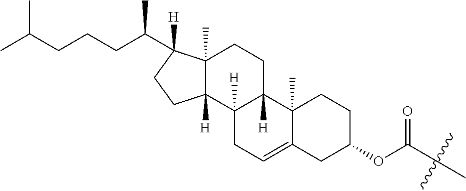 |
| Lipid 5 | 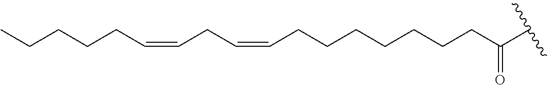 [SMILES: *C(=O)CCCCCCC/C=C\C/C=C\CCCCC] |
| Lipid 6 | 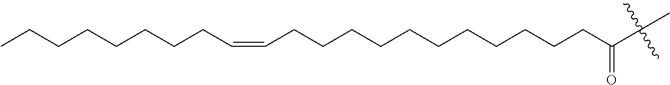 [SMILES: *C(=O)CCCCCCCCCCC/C=C\CCCCCCCC] |
| Lipid 7 | 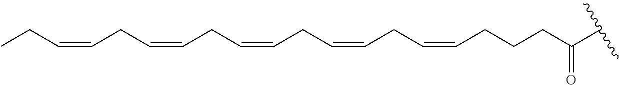 [SMILES: *C(=O)CCC/C=C\C/C=C\C/C=C\C/C=C\C/C=C\CC] |
| Lipid 10 | 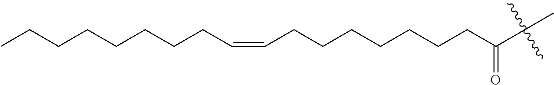 [SMILES: *C(=O)CCCCCCC/C=C\CCCCCCCC] |
| Lipid 12 | 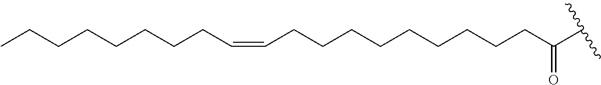 [SMILES: *C(=O)CCCCCCCCC/C=C\CCCCCCCC] |
| Lipid 19 | 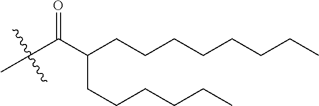 [SMILES: *C(=O)C(CCCCCC)CCCCCCCC] |

wherein [symbol] indicates a point of connection to L₂₁ or L₂₂.

In some embodiments, L_{A2} comprises at least one PEG unit. In some embodiments, L_{A2} is free of any PEG units. In some embodiments, L_{A2} comprises —C(O)—, —C(O)NH—, optionally substituted alkoxy, or an optionally substituted alkyleneheterocyclyl. In some embodiments, L_{A2} is a bond.

In some embodiments, L_{A2} is selected from the group consisting of the moieties identified in Table 12.

TABLE 12
Example L_{A2} moieties of the present invention.

| Name | Structure |
|---|---|
| Tether 1-2 | 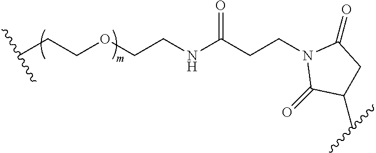 |
| Tether 2-2 | 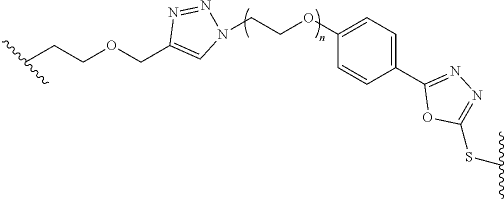 |
| Tether 3-2 | 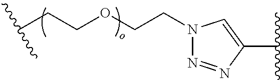 |

wherein each of m, n, and o is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to the RNAi agent or —C(O)—.

In some embodiments, m is 1, 2, 3, 4, 5, 6, 7, 8, 9, 10, 20, 21, 22, 23, or 25. In some embodiments, m is 2, 4, 8, or 24. In some embodiments, each n is 2, 3, 4, or 5. In some embodiments, n is 4. In some embodiments, o is 2, 3, 4, 5, 6, 7, 8, 9, 10, 11, 12, or 13. In some embodiments, o is 4, 8, or 12.

In some embodiments, each of R¹, R² and R³ is independently hydrogen or C₁₋₃ alkyl. In some embodiments, each of R¹, R² and R³ is hydrogen.

In some embodiments, the lipid PK/PD modulator of Formula (II) is selected from the group consisting of LP 38a, LP 39a, LP 43a, LP 44a, LP 45a, LP 47a, LP 53a, LP 54a, LP 55a, LP 57a, LP 58a, LP 59a, LP 62a, LP 101a, LP 104a, and LP 111a as set forth in Table 19, or a pharmaceutically acceptable salt of any of these lipid PK/PD modulators, wherein each L_{AA} is a bond or a bivalent moiety connecting the RNAi agent to the rest of the lipid PK/PD modulator, and each [symbol] indicates a point of connection to the RNAi agent.

In some embodiments, the lipid PK/PD modulator of Formula (II) is selected from the group consisting of LP 38b, LP 39b, LP 41b, LP 42b, LP 43b, LP 44b, LP 45b, LP 47b, LP 53b, LP 54b, LP 55b, LP 57b, LP 58b, LP 59b, LP 60b, LP 62b, LP 101b, LP 104b, LP 106b, LP 107b, LP 108b, LP 109b, and LP 111b as set forth in Table 21, or a pharmaceutically acceptable salt of any of these lipid PK/PD modulators, wherein each [symbol] indicates a point of connection to the RNAi agent.

Another aspect of the present invention provides a lipid PK/PD modulator of Formula (III):

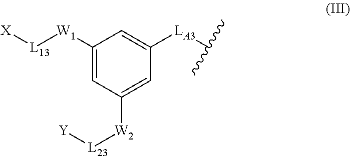

or a pharmaceutically acceptable salt thereof, wherein X and Y are as defined for any embodiments of the lipid PK/PD modulator of Formula (I), (Ia), (Ib), (Ib1), (Ic), (Id) or (II);
L₁₃ is L₁ as defined for any embodiments of the lipid PK/PD modulator of Formula (I), (Ia), (Ib), (Ib1), (Ic), or (Id), or L₁₃ is Liz as defined for any embodiments of the lipid PK/PD modulator of Formula (II); L₂₃ is L₂ as defined for any embodiments of the lipid PK/PD modulator of Formula (I), (Ia), (Ib), (Ib1), (Ic), or (Id), or L₂₃ is L₂₂ as defined for any embodiments of the lipid PK/PD modulator of Formula (II); W₁ is —C(O)NR₁— or —OCH₂CH₂NR₁C(O)—, wherein R₁ is hydrogen or C₁₋₆ alkyl; W₂ is —C(O)NR₂— or —OCH₂CH₂NR₂C(O)—, wherein R² is hydrogen or C₁₋₆ alkyl; L_{A3} is L_{A} as defined for any embodiments of the lipid PK/PD modulator of Formula (I), (Ia), (Ib), (Ib1), or (Ic), or L_{A3} is L_{A2} as defined for any embodiments of the lipid PK/PD modulator of Formula (II); and [symbol] indicates a point of connection to the RNAi agent.

In some embodiments, L_{A3} is a bond or a bivalent moiety connecting the RNAi agent to the phenyl ring; W₁ is —C(O)NR₁— or —OCH₂CH₂NR₁C(O)—, wherein R₁ is hydrogen or C₁₋₆ alkyl; W₂ is —C(O)NR₂— or —OCH₂CH₂NR₂C(O)—, wherein R² is hydrogen or C₁₋₆ alkyl; L₁₃ and L₂₃ are each independently linkers comprising at least about 5 PEG units; X and Y are each independently lipids comprising from about 10 to about 50 carbon atoms; and [symbol] indicates a point of connection to the RNAi agent

In some embodiments, each of L₁₃ and L₂₃ is independently selected from the group consisting of the moieties identified in Table 13.

TABLE 13
Example L₁₃ and L₂₃ moieties of the present invention.

| Name | Structure |
|---|---|
| Linker 1-3 | 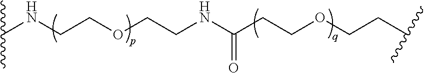 |
| Linker 2-3 | 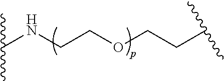 |
| Linker 3-3 | 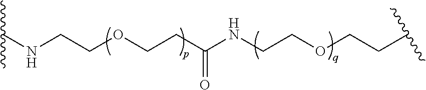 |

wherein, p and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and each [symbol] indicates a point of connection to X, Y, W₁, or W₂; provided that: (i) in Linker 1-3 and Linker 3-3, p + q ≥ 5; and (ii) in Linker 2-3, p ≥ 5.

In some embodiments, each p is independently 20, 21, 22, 23, 24, or 25. In some embodiments, each p is independently 23 or 24. In some embodiments, each p is 23. In some embodiments, each p is 24. In some embodiments, each q is independently 20, 21, 22, 23, 24, or 25. In some embodiments, each q is 24.

In some embodiments, at least one of X and Y is selected from the group consisting of the moieties identified in Table 8, wherein each [symbol] indicates a point of connection to L₁₃ or L₂₃. In some embodiments, each of X and Y is independently selected from the group consisting of the moieties identified in Table 8, wherein each [symbol] indicates a point of connection to L₁₃ or L₂₃.

In some embodiments, at least one of X and Y is selected from the group consisting of the moieties identified in Table 14. In some embodiments, each of X and Y is independently selected from the group consisting of the moieties identified in Table 14.

TABLE 14
Example X and Y moieties of the lipid PK/PD modulator of Formula (III).

| Name | Structure |
|---|---|
| Lipid 3 | 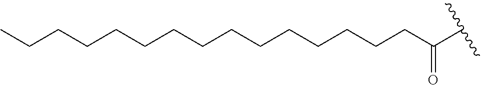 [SMILES: *C(=O)CCCCCCCCCCCCCCC] |
| Lipid 19 | 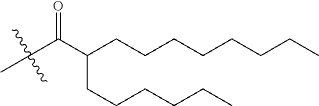 [SMILES: *C(=O)C(CCCCCC)CCCCCCCC] |

wherein [symbol] indicates a point of connection to L₁₃ or L₂₃.

In some embodiments, L_{A3} comprises at least one PEG unit. In some embodiments, L_{A3} is free of any PEG units. In some embodiments, L_{A3} comprises —C(O)—, —C(O)NH—, optionally substituted alkoxy, or an optionally substituted alkyleneheterocyclyl. In some embodiments, L_{A3} is a bond.

In some embodiments, L_{A3} is selected from the group consisting of the moieties identified in Table 15.

TABLE 15
Example L_{A3} moieties of the present invention.

| Name | Structure |
|---|---|
| Tether 1-3 | 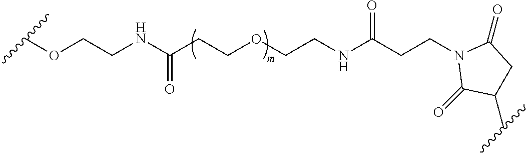 |
| Tether 2-3 | 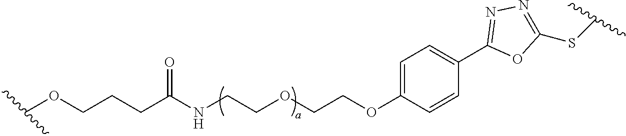 |
| Tether 3-3 | 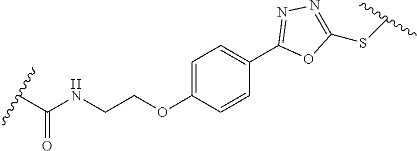 [SMILES: *Sc1nnc(-c2ccc(OCCNC(*)=O)cc2)o1] |
| Tether 4-3 | 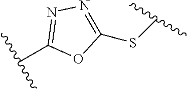 [SMILES: *Sc1nnc(*)o1] |
| Tether 5-3 | 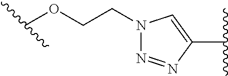 [SMILES: *OCCn1cc(*)nn1] |

wherein, each of m and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and each [symbol] indicates a point of connection to the RNAi agent or the phenyl ring of Formula (III).

In some embodiments, m is 1, 2, 3, 4, 5, 20, 21, 22, 23, or 25. In some embodiments, m is 1, 2, 3, 4, or 5. In some embodiments, m is 2 or 4. In some embodiments, a is 2, 3, 4, or 5. In some embodiments, a is 3.

In some embodiments, each of R¹ and R² is independently hydrogen or C₁₋₃ alkyl (e.g., methyl, ethyl, or n-propyl). In some embodiments, both of R¹ and R² is hydrogen.

In some embodiments, the lipid PK/PD modulator of Formula (III) is selected from the group consisting of LP 110a, LP 124a, LP 130a, and LP 220a as set forth in Table 19, or a pharmaceutically acceptable salt of any of these lipid PK/PD modulators, wherein each L_{AA} is a bond or a bivalent moiety connecting the RNAi agent to the rest of the lipid PK/PD modulator; and each [symbol] indicates a point of connection to the RNAi agent.

In some embodiments, the lipid PK/PD modulator of Formula (III) is selected from the group consisting of LP 110b, LP 124b, LP 130b, LP 143b, LP 220b, LP 221b, and LP 240b as set forth in Table 21, or a pharmaceutically acceptable salt of any of these lipid PK/PD modulators, wherein each [symbol] indicates a point of connection to the RNAi agent.

Another aspect of the present invention provides a lipid PK/PD modulator of Formula (IIIa):

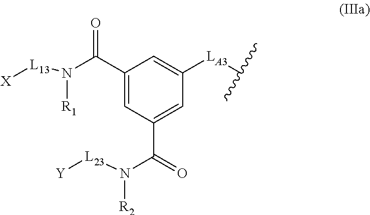

or a pharmaceutically acceptable salt thereof, wherein X and Y are as defined for any embodiments of the lipid PK/PD modulator of Formula (I), (Ia), (Ib), (Ib1), (Ic), (Id), (II), or (III); L₁₃ is L₁ as defined for any embodiments of the lipid PK/PD modulator of Formula (I), (Ia), (Ib), (Ib1), (Ic), or (Id), L₁₃ is L₁₂ as defined for any embodiments of the lipid PK/PD modulator of Formula (II), or L₁₃ is as defined in any embodiments of the lipid PK/PD modulator of Formula (III); L₂₃ is L₂ as defined for any embodiments of the lipid PK/PD modulator of Formula (I), (Ia), (Ib), (Ib1), (Ic), or (Id), L₂₃ is L₂₂ as defined for any embodiments of the lipid PK/PD modulator of Formula (II), or L₁₃ is as defined in any embodiments of the lipid PK/PD modulator of Formula (III); L_{A3} is L_{A} as defined for any embodiments of the lipid PK/PD modulator of Formula (I), (Ia), (Ib), (Ib1), or (Ic), L_{A3} is L_{A2} as defined for any embodiments of the lipid PK/PD modulator of Formula (II), or L_{A3} is as defined for any embodiments of the lipid PK/PD modulator of Formula (III); each of R₁ and R² are as defined in any embodiments of the lipid PK/PD modulator of Formula (II) or (III); and [symbol] indicates a point of connection to the RNAi agent.

In some embodiments, L_{A3} is a bond or a bivalent moiety connecting the RNAi agent to the phenyl ring; R₁ and R² are each independently hydrogen or C₁₋₆ alkyl (e.g., methyl, ethyl, n-propyl, n-butyl, or n-pentyl); L₁₃ and L₂₃ are each independently linkers comprising at least about 5 PEG units; X and Y are each independently lipids comprising from about 10 to about 50 carbon atoms; and [symbol] indicates a point of connection to the RNAi agent.

In some embodiments, each of L₁₃ and L₂₃ is selected from the group consisting of Linker 1-3 and Linker 2-3 as set forth in Table 9, wherein each [symbol] indicates a point of connection to X, Y, —NR₁—, or —NR₂— in Formula (IIIa), provided that:
(i) in Linker 1-3, p+q≥5; and
(ii) in Linker 2-3, p≥5.

In some embodiments, one of L₁₃ and L₂₃ is Linker 1-3 and the other is Linker 2-3. In some embodiments, each of L₁₃ and L₂₃ is Linker 1-3. In some embodiments, each of L₁₃ and L₂₃ is Linker 2-3.

In some embodiments, each p is independently 23 or 24. In some embodiments, each p is 23. In some embodiments, each p is 24. In some embodiments, q is 24.

In some embodiments, at least one of X and Y is selected from the group consisting of Lipid 3 and Lipid 19 as set forth in Table 10, wherein each [symbol] indicates a point of connection to L₁₃ or L₂₃ in Formula (IIIa). In some embodiments, each of X and Y is independently selected from the group consisting of Lipid 3 and Lipid 19. In some embodiments, one of X and Y is Lipid 3 and the other is Lipid 19. In some embodiments, each of X and Y is Lipid 3. In some embodiments, each of X and Y is Lipid 19.

In some embodiments, L_{A3} is selected from the group consisting of Tether 1-3, Tether 2-3, and Tether 5-3 as set forth in Table 15, wherein each [symbol] indicates a point of connection to the RNAi agent or the phenyl ring of Formula (IIIa). In some embodiments, L_{A3} is Tether 1-3. In some embodiments, L_{A3} is Tether 2-3. In some embodiments, L_{A3} is Tether 5-3.

In some embodiments, m is 1, 2, 3, 4, 5, 20, 21, 22, 23, or 25. In some embodiments, m is 1, 2, 3, 4, or 5. In some embodiments, m is 2 or 4. In some embodiments, a is 2, 3, 4, or 5. In some embodiments, a is 3.

In some embodiments, each of R¹ and R² is independently hydrogen or C₁₋₃ alkyl. In some embodiments, each of R¹ and R² is hydrogen.

In some embodiments, the lipid PK/PD modulator of Formula (IIIa) is selected from the group consisting of LP 110a, LP 124a, and LP 130a as set forth in Table 19 or a pharmaceutically acceptable salt of any of these lipid PK/PD modulators, wherein each L_{AA} is a bond or a bivalent moiety connecting the RNAi agent to the rest of the lipid PK/PD modulator; and each [symbol] indicates a point of connection to the RNAi agent.

In some embodiments, the lipid PK/PD modulator of Formula (IIIa) is selected from the group consisting of LP 110b, LP 124b, LP 130b, LP 143b, and LP 240b as set forth in Table 21, or a pharmaceutically acceptable salt of any of these lipid PK/PD modulators, wherein each [symbol] indicates a point of connection to the RNAi agent.

Another aspect of the present invention provides a lipid PK/PD modulator of Formula (IIIb):

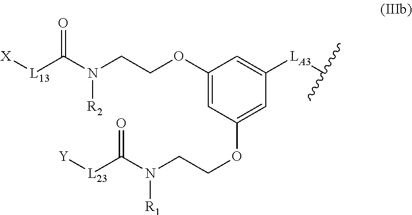

or a pharmaceutically acceptable salt thereof, wherein X and Y are as defined for any embodiments of the lipid PK/PD modulator of Formula (I), (Ia), (Ib), (Ib1), (Ic), (Id), (II), (III), or (IIIa); L₁₃ is L₁ as defined for any embodiments of the lipid PK/PD modulator of Formula (I), (Ia), (Ib), (Ib1), (Ic), or (Id), L₁₃ is Liz as defined for any embodiments of the lipid PK/PD modulator of Formula (II), or L₁₃ is as defined in any embodiments of the lipid PK/PD modulator of Formula (III) or (IIIa); L₂₃ is L₂ as defined for any embodiments of the lipid PK/PD modulator of Formula (I), (Ia), (Ib), (Ib1), (Ic), or (Id), L₂₃ is L₂₂ as defined for any embodiments of the lipid PK/PD modulator of Formula (II), or L₁₃ is as defined in any embodiments of the lipid PK/PD modulator of Formula (III) or (IIIa); L_{A3} is L_{A} as defined for any embodiments of the lipid PK/PD modulator of Formula (I), (Ia), (Ib), (Ib1), or (Ic), L_{A3} is L_{A2} as defined for any embodiments of the lipid PK/PD modulator of Formula (II), or L_{A3} is as defined for any embodiments of the lipid PK/PD modulator of Formula (III) or (IIIa); each of R₁ and R² are as defined in any embodiments of the lipid PK/PD modulator of Formula (II), (III), or (IIIa); and [symbol] indicates a point of connection to the RNAi agent.

In some embodiments, L_{A3} is a bond or a bivalent moiety connecting the RNAi agent to the phenyl ring; R₁ and R² are each independently selected from hydrogen or C₁₋₆ alkyl; L₁₃ and L₂₃ are each independently linkers comprising at least about 5 PEG units; X and Y are each independently lipids comprising from about 10 to about 50 carbon atoms; and [symbol] indicates a point of connection to the RNAi agent.

In some embodiments, each of L₁₃ and L₂₃ is Linker 3-3 as set forth in Table 13, wherein each [symbol] indicates a point of connection to X, Y, or —C(O)—, provided that in Linker 3-3,p+q≥5.

In some embodiments, p is 23 or 24. In some embodiments, p is 23. In some embodiments, p is 24. In some embodiments, q is 24.

In some embodiments, each of X and Y is Lipid 3 as set forth in Table 14, wherein each [symbol] indicates a point of connection to L₁₃ or L₂₃.

In some embodiments, L_{A3} is selected from the group consisting of Tether 3-3 and Tether 4-3 as set forth in Table 15, wherein each [symbol] indicates a point of connection to the RNAi agent or the phenyl ring of Formula (IIIb). In some embodiments, L_{A3} is Tether 3-3. In some embodiments, L_{A3} is Tether 4-3.

In some embodiments, each of R¹ and R² is independently hydrogen or C₁₋₃ alkyl. In some embodiments, each of R¹ and R² is hydrogen.

In some embodiments, the lipid PK/PD modulator of Formula (IIIb) is LP 220a as set forth in Table 19, or a pharmaceutically acceptable salt thereof, wherein L_{AA} is a bond or a bivalent moiety connecting the RNAi agent to the rest of the lipid PK/PD modulator; and [symbol] indicates a point of connection to the RNAi agent.

In some embodiments, the lipid PK/PD modulator of Formula (IIIb) is selected from the group consisting of LP 220b and LP 221b as set forth in Table 21, or a pharmaceutically acceptable salt of any of these lipid PK/PD modulators, wherein each [symbol] indicates a point of connection to the RNAi agent.

Another aspect of the invention provides a lipid PK/PD modulator of Formula (IV):

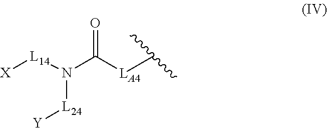

or a pharmaceutically acceptable salt thereof, wherein X and Y are as defined for any embodiments of the lipid PK/PD modulator of Formula (I), (Ia), (Ib), (Ib1), (Ic), (Id), (II), (III), (IIIa), or (IIIb); L₁₄ is L₁ as defined for any embodiments of the lipid PK/PD modulator of Formula (I), (Ia), (Ib), (Ib1), (Ic), or (Id), L₁₄ is Lie as defined for any embodiments of the lipid PK/PD modulator of Formula (II), or L₁₄ is L₁₃ as defined in any embodiments of the lipid PK/PD modulator of Formula (III), (IIIa), or (IIIb); L₂₄ is L₂ as defined for any embodiments of the lipid PK/PD modulator of Formula (I), (Ia), (Ib), (Ib1), (Ic), or (Id), L₂₄ is L₂₂ as defined for any embodiments of the lipid PK/PD modulator of Formula (II), or L₂₄ is L₂₃ as defined in any embodiments of the lipid PK/PD modulator of Formula (III), (IIIa), or (IIIb): L_{A4} is L_{A} as defined for any embodiments of the lipid PK/PD modulator of Formula (I), (Ia), (Ib), (Ib1), or (Ic), L_{A4} is L_{A2} as defined for any embodiments of the lipid PK/PD modulator of Formula (II), or L_{A4} is L_{A3} as defined for any embodiments of the lipid PK/PD modulator of Formula III(Ma), or (Mb); and [symbol] indicates a point of connection to the RNAi agent.

In some embodiments, L_{A4} is a bond or a bivalent moiety connecting the RNAi agent to —C(O)—; L₁₄ and L₂₄ are each independently linkers comprising at least about 5 PEG units; X and Y are each independently lipids comprising from about 10 to about 50 carbon atoms; and [symbol] indicates a point of connection to the RNAi agent.

In some embodiments, each of L₁₄ and L₂₄ is independently selected from the group consisting of the moieties identified in Table 16.

TABLE 16
Example L₁₄ and L₂₄ moieties of the present invention.

| Name | Structure |
|---|---|
| Linker 1-4 | 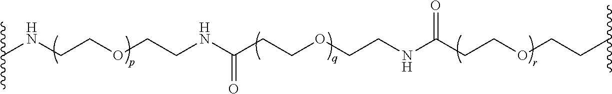 |
| Linker 2-4 | 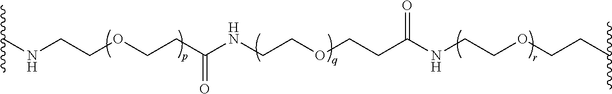 |
| Linker 3-4 | 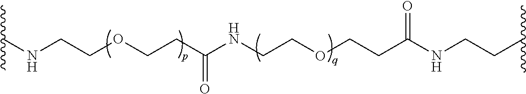 |
| Linker 4-4 | 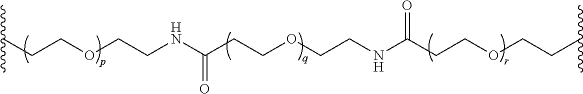 |
| Linker 5-4 | 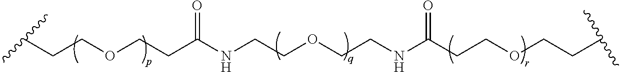 |

wherein each p is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each q is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; each r is independently 1, 2, 3, 4, 5, 6, 7, 8, 9, or 10; and each [symbol] indicates a point of connection to X, Y, or 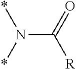 of Formula (IV), wherein each * indicates the point of attachment to L₁₄ or L₂₄; provided that: (i) in Linker 1-4, Linker 2-4, and Linker 4-4,
p + q + r ≥ 5; and (ii) in Linker 3-4, p + q ≥ 5.

In some embodiments, each p is independently 20, 21, 22, 23, 24, or 25. In some embodiments, each p is independently 23 or 24. In some embodiments, each p is 23. In some embodiments, each p is 24. In some embodiments, each q is independently 20, 21, 22, 23, 24, or 25. In some embodiments, each q is independently 23 or 24. In some embodiments, each q is 24. In some embodiments, each q is 23. In some embodiments, r is 2, 3, 4, 5, or 6. In some embodiments, each r is 4.

In some embodiments, at least one of X and Y is selected from the group consisting of the moieties identified in Table 8, wherein each [symbol] indicates a point of connection to L₁₄ or L₂₄. In some embodiments, each of X and Y is independently selected from the group consisting of the moieties identified in Table 8, wherein each [symbol] indicates a point of connection to L₁₄ or L₂₄.

In some embodiments, at least one of X and Y is selected from the group consisting of the moieties identified in Table 17. In some embodiments, each of X and Y is independently selected from the group consisting of the moieties identified in Table 17.

TABLE 17
Example X and Y moieties of the lipid PK/PD modulator of Formula
(IV).

| Name | Structure |
|---|---|
| Lipid 1 | 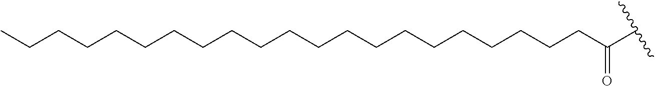 [SMILES: *C(=O)CCCCCCCCCCCCCCCCCCCCC] |
| Lipid 2 | 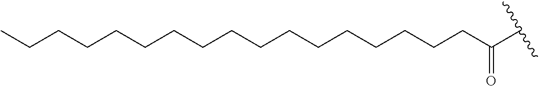 [SMILES: *C(=O)CCCCCCCCCCCCCCCCC] |
| Lipid 3 | 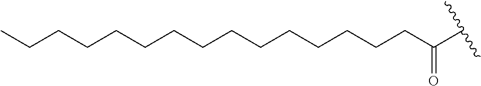 [SMILES: *C(=O)CCCCCCCCCCCCCCC] |
| Lipid 5 | 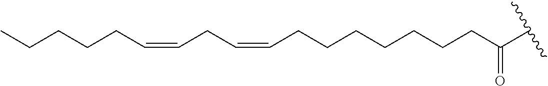 [SMILES: *C(=O)CCCCCCC/C=C\C/C=C\CCCCC] |
| Lipid 8 | 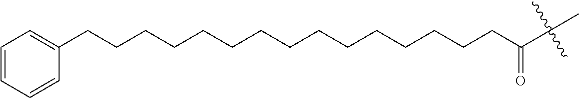 [SMILES: *C(=O)CCCCCCCCCCCCCCCc1ccccc1] |
| Lipid 9 | 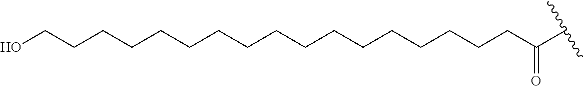 [SMILES: *C(=O)CCCCCCCCCCCCCCCCCO] |
| Lipid 10 | 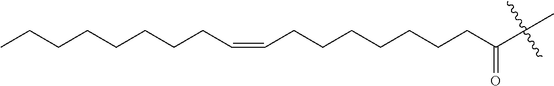 [SMILES: *C(=O)CCCCCCC/C=C\CCCCCCCC] |
| Lipid 11 | 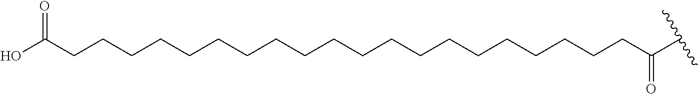 [SMILES: *C(=O)CCCCCCCCCCCCCCCCCCCCC(=O)O] |
| Lipid 12 | 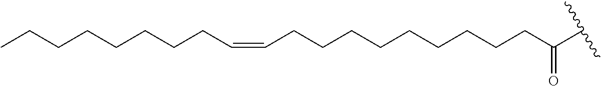 [SMILES: *C(=O)CCCCCCCCC/C=C\CCCCCCCC] |
| Lipid 15 | 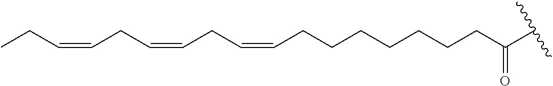 [SMILES: *C(=O)CCCCCCC/C=C\C/C=C\C/C=C\CC] |
| Lipid 16 | 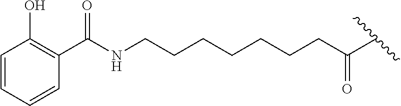 [SMILES: *C(=O)CCCCCCCNC(=O)c1ccccc1O] |
| Lipid 17 | 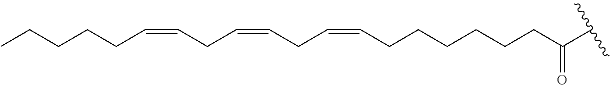 [SMILES: *C(=O)CCCCCC/C=C\C/C=C\C/C=C\CCCCC] |
| Lipid 18 | 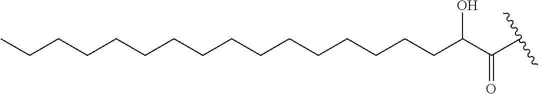 [SMILES: *C(=O)C(O)CCCCCCCCCCCCCCCC] |
| Lipid 19 | 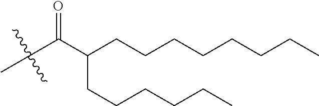 [SMILES: *C(=O)C(CCCCCC)CCCCCCCC] |
| Lipid 20 | 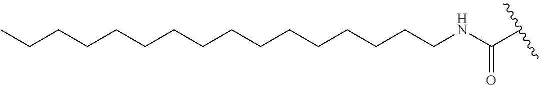 [SMILES: *C(=O)NCCCCCCCCCCCCCCCC] |
| Lipid 21 | 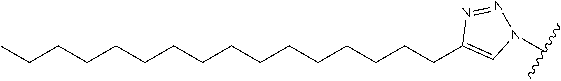 [SMILES: *n1cc(CCCCCCCCCCCCCCCC)nn1] |
| Lipid 22 | 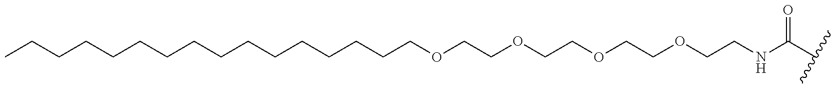 [SMILES: *C(=O)NCCOCCOCCOCCOCCCCCCCCCCCCCCCC] |
| Lipid 23 | 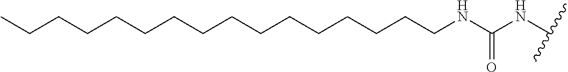 [SMILES: *NC(=O)NCCCCCCCCCCCCCCCC] |
| Lipid 24 | 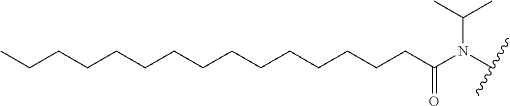 [SMILES: *N(C(=O)CCCCCCCCCCCCCCC)C(C)C] |

wherein [symbol] indicates a point of connection to L₁₄ or L₂₄.

In some embodiments, L_{A4} comprises at least one PEG unit. In some embodiments, L_{A4} is free of any PEG units. In some embodiments, L_{A4} comprises —C(O)—, —C(O)NH—, optionally substituted alkoxy, or an optionally substituted alkyleneheterocyclyl. In some embodiments, L_{A4} is a bond.

In some embodiments, L_{A4} is selected from the group consisting of the moieties identified in Table 18.

TABLE 18
Example L_{A4} moieties of the present invention.

| Name | Structure |
|---|---|
| Tether 1-4 | 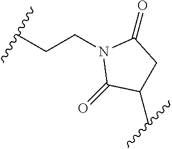 [SMILES: *CCN1C(=O)CC(*)C1=O] |
| Tether 2-4 | 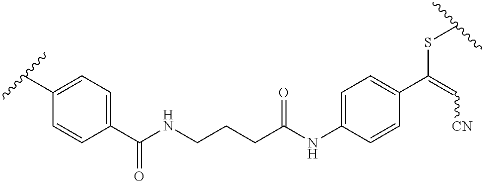 [SMILES: *SC(=CC#N)c1ccc(NC(=O)CCCNC(=O)c2ccc(*)cc2)cc1] |
| Tether 3-4 | 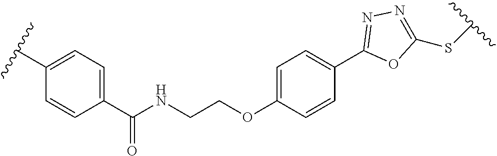 [SMILES: *Sc1nnc(-c2ccc(OCCNC(=O)c3ccc(*)cc3)cc2)o1] |
| Tether 4-4 | 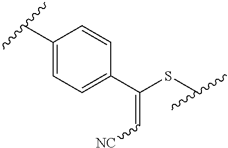 [SMILES: *SC(=CC#N)c1ccc(*)cc1] |
| Tether 5-4 | 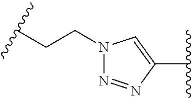 [SMILES: *CCn1cc(*)nn1] |
| Tether 6-4 | 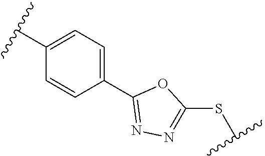 [SMILES: *Sc1nnc(-c2ccc(*)cc2)o1] |

wherein each [symbol] indicates a point of connection to the RNAi agent or the —C(O)— of Formula (IV).

In some embodiments, the lipid PK/PD modulator of Formula (IV) is selected from the group consisting of LP 1a, LP 28a, LP 29a, LP 48a, LP 49a, LP 56a, LP 61a, LP 87a, LP 89a, LP 90a, LP 92a, LP 93a, LP 94a, LP 95a, LP 102a, LP 103a, LP 223a, LP 225a, LP 246a, LP 339a, LP 340a, LP 357a, and LP 358a as set forth in Table 15, or a pharmaceutically acceptable salt of any of these lipid PK/PD modulators, wherein each L_{AA} is a bond or a bivalent moiety connecting the RNAi agent to the rest of the lipid PK/PD modulator; and each [symbol] indicates a point of connection to the RNAi agent.

In some embodiments, the lipid PK/PD modulator of Formula (IV) is selected from the group consisting of LP 1b, LP 28b, LP 29b, LP 48b, LP 49b, LP 56b, LP 61b, LP 87b, LP 89b, LP 90b, LP 92b, LP 93b, LP 94b, LP 95b, LP 102b, LP 103b, LP 223b, LP 224b, LP 225b, LP 226b, LP 238b, LP 246b, LP 247b, LP 339b, LP 340b, LP 357b, and LP 358b as set forth in Table 17, or a pharmaceutically acceptable salt of any of these lipid PK/PD modulators, wherein each [symbol] indicates a point of connection to the RNAi agent.

Another aspect of the invention provides a compound of Formula (IVa):

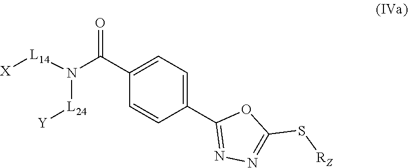

or a pharmaceutically acceptable salt thereof, wherein X and Y are as defined for any embodiments of the compound of Formula (I), (Ia), (Ib), (Ib1), (Ic), (II), (III), (IIIa), (IIIb), or (IV); L₁₄ and L₂₄ are as defined in any of the embodiments of the compound of Formula (IV); and Rz comprises an oligonucleotide-based agent.

In some embodiments, Rz comprises an oligonucleotide-based agent; each of L₁₄ and L₂₄ is independently selected from the group consisting of

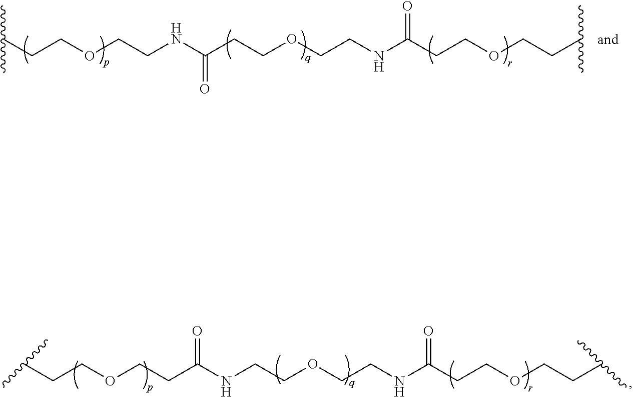

wherein each [symbol] indicates a point of connection to X, Y, or

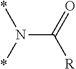

of Formula (IVa), each * indicates the point of attachment to L₁₄ or L₂₄, each p is independently 20, 21, 22, 23, 24, or 25, each q is independently 20, 21, 22, 23, 24, or 25, and each r is independently 2, 3, 4, 5, or 6; and each of X and Y is independently selected from the group consisting of

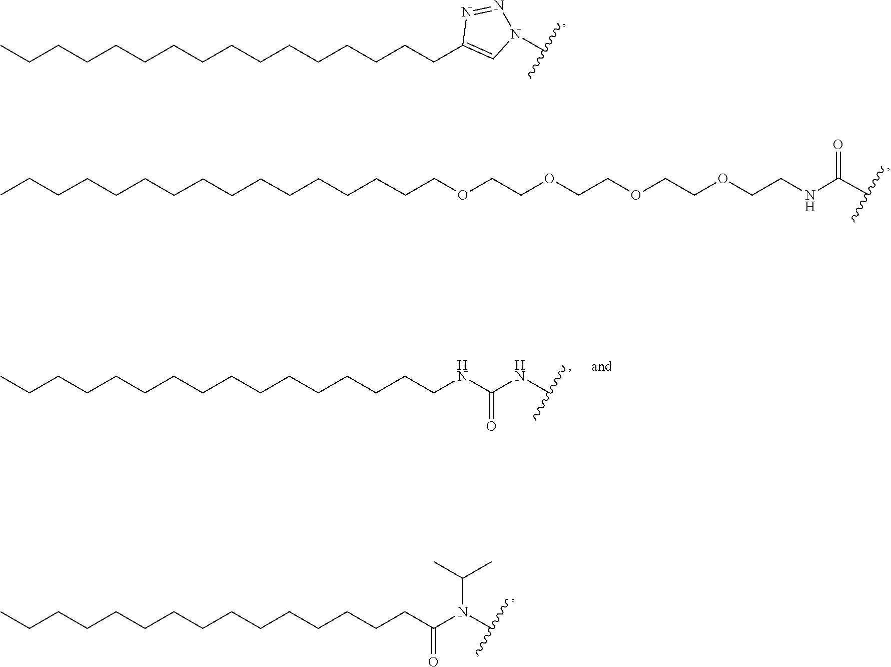 [SMILES: *n1cc(CCCCCCCCCCCCCCCC)nn1 ; *C(=O)NCCOCCOCCOCCOCCCCCCCCCCCCCCCC ; *NC(=O)NCCCCCCCCCCCCCCCC ; *N(C(=O)CCCCCCCCCCCCCCC)C(C)C]

wherein [symbol] indicates a point of connection to L₁₄ or L₂₄.

In some embodiments, each p is independently 23 or 24. In some embodiments, each p is 23. In some embodiments, each p is 24. In some embodiments, each q is independently 23 or 24. In some embodiments, each q is 24. In some embodiments, each q is 23. In some embodiments, each r is 4.

In some embodiments, the compound of Formula (IVa) is selected from the group consisting of LP 339b, LP 340b, LP 357b, and LP 358b as set forth in Table 16, or a pharmaceutically acceptable salt of any of these compounds, wherein each Rz comprises an oligonucleotide-based agent.

In another aspect of the invention, the RNAi agent may be conjugated to a lipid PK/PD modulator selected from the group consisting of the lipid PK/PD modulators identified in Table 19.

TABLE 19
Example lipid PK/PD modulators of the present invention (compound
number appears before structure).
LP 1a

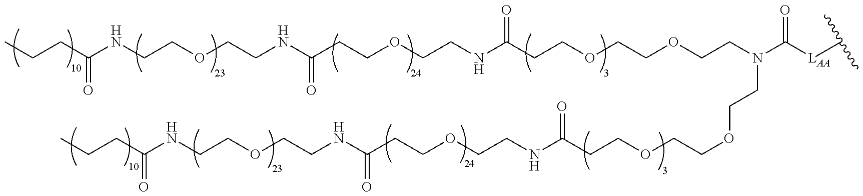

LP 28a

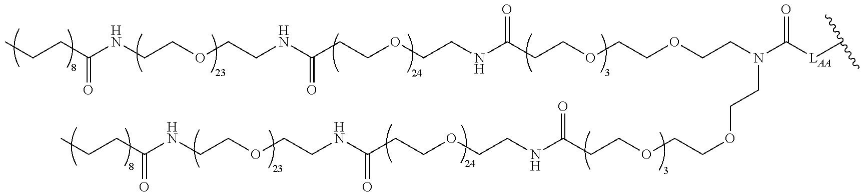

LP 29a

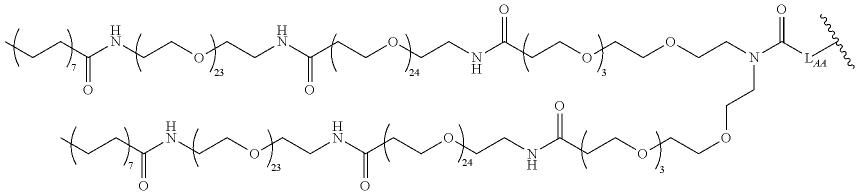

LP 38a

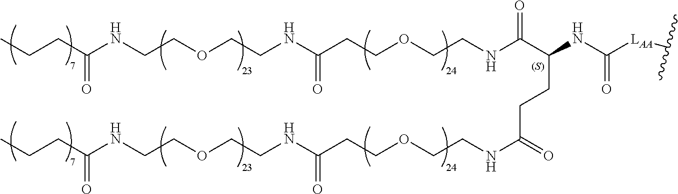

LP 39a

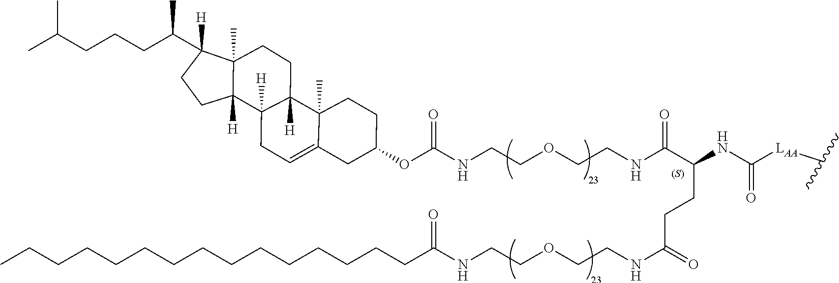

LP 43a

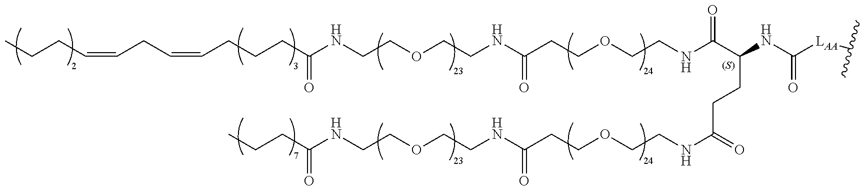

LP 44a

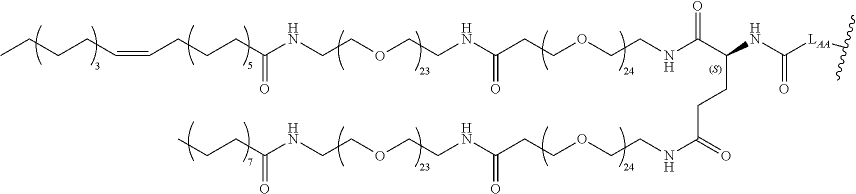

LP 45a

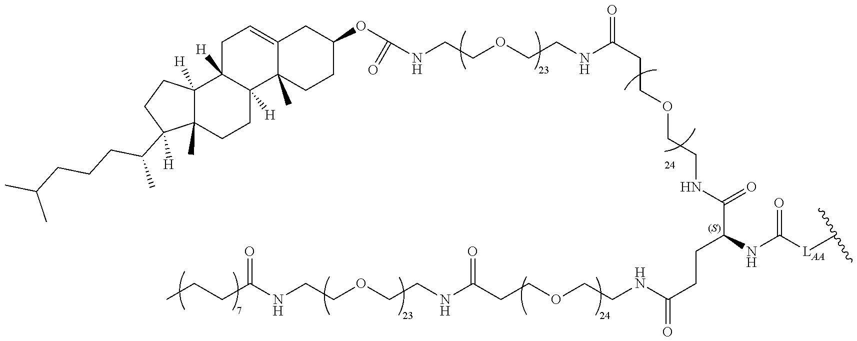

LP 47a

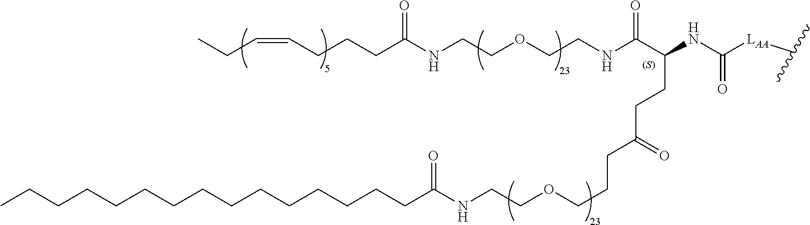

LP 48a

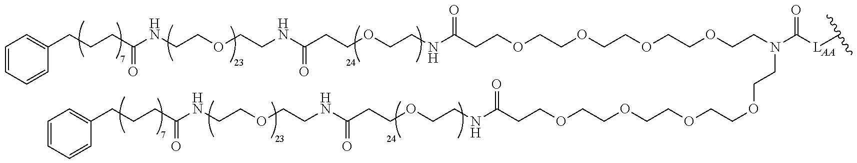

LP 49a

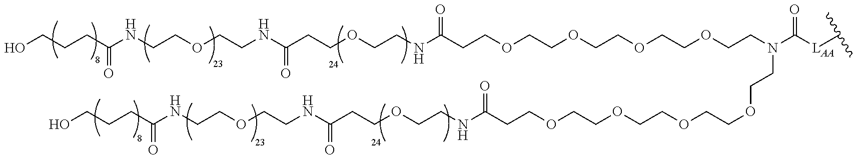

LP 53a

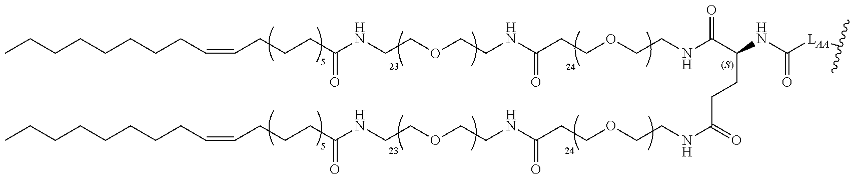

LP 54a

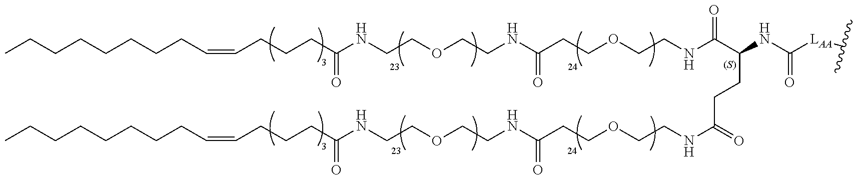

LP 55a

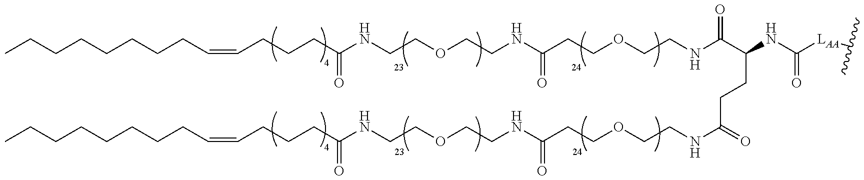

LP 56a

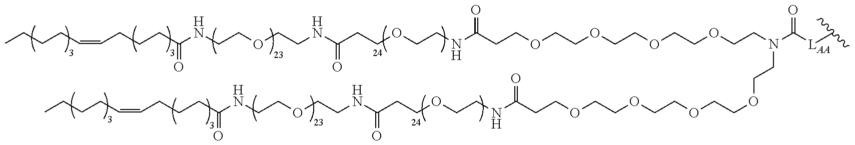

LP 57a

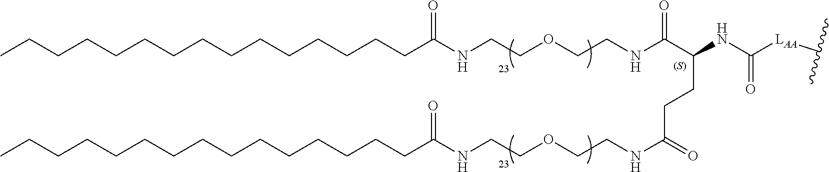

LP 58a

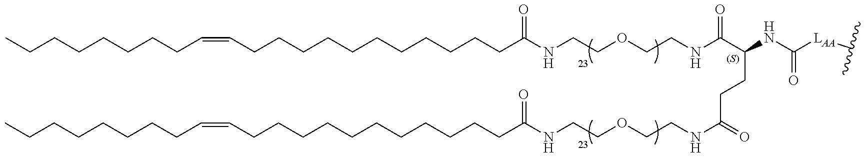

LP 59a

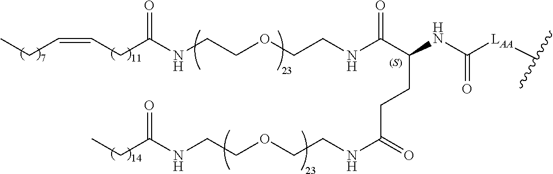

LP 61a

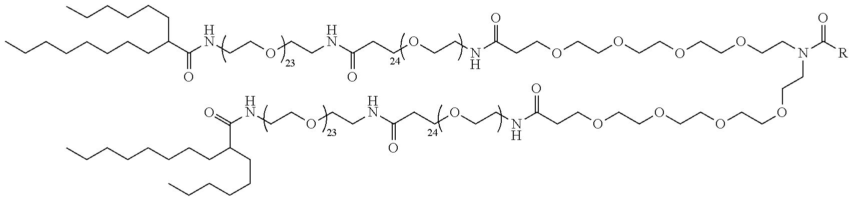

LP 62a

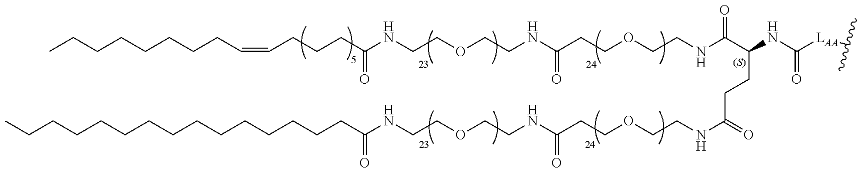

LP 87a

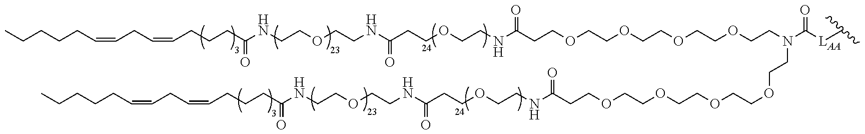

LP 89a

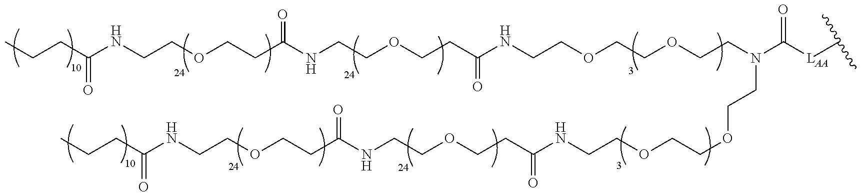

LP 90a

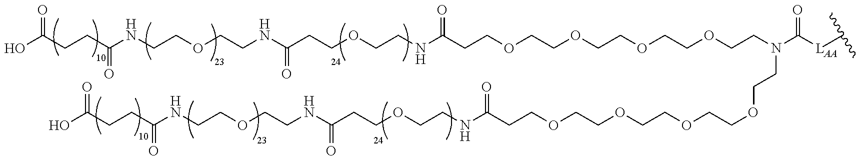

LP 92a

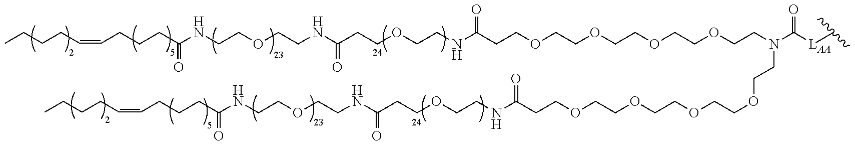

LP 93a

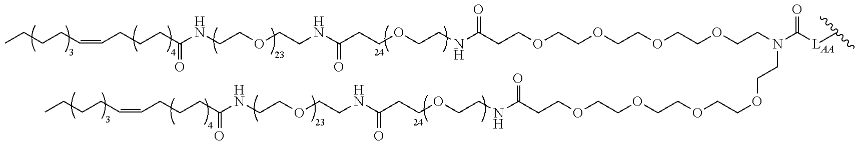

LP 94a

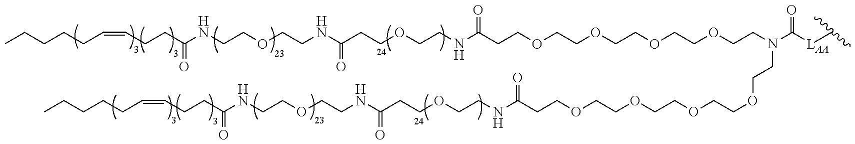

LP 95a

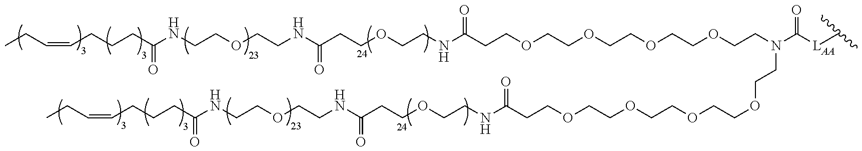

LP 101a

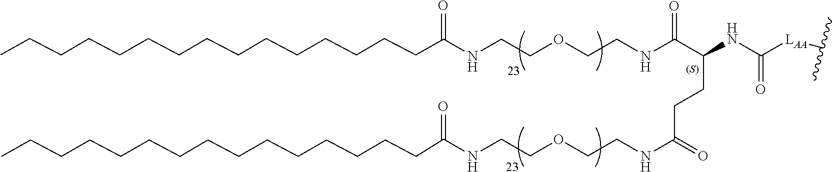

LP 102a

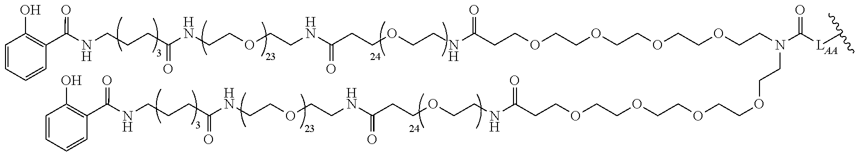

LP 103a

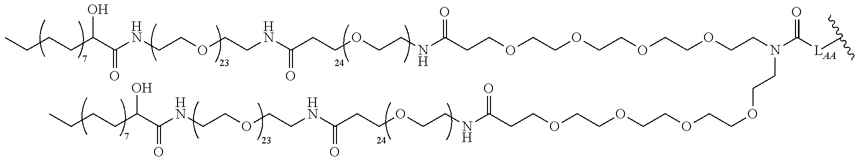

LP 104a

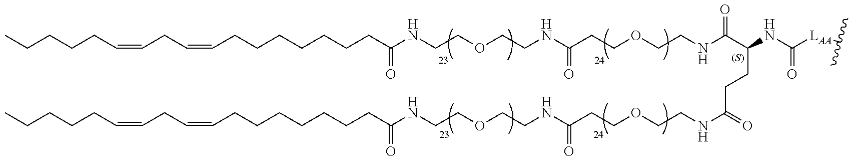

LP 110a

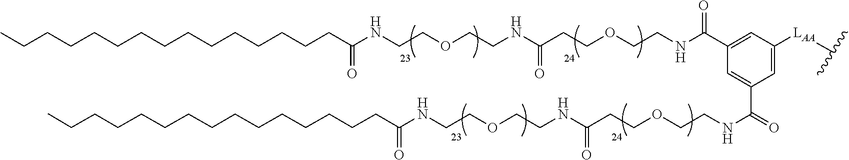

LP 111a

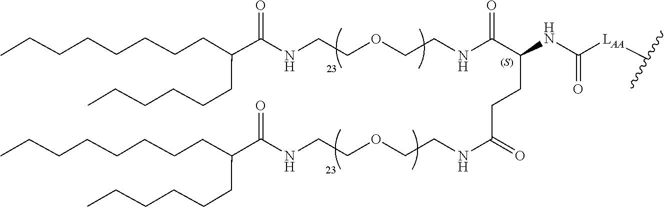

LP 124a

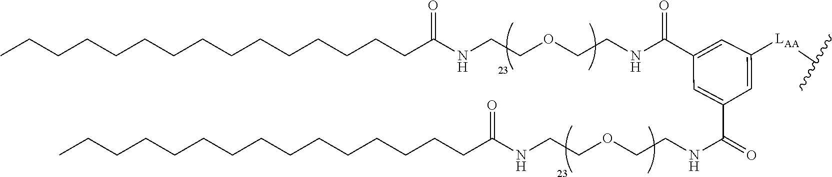

LP 130a

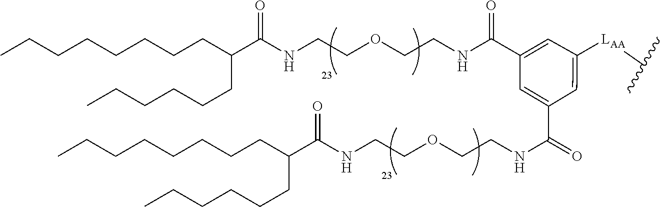

LP 210a

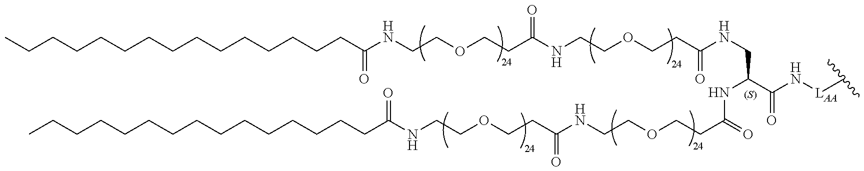

LP 217a

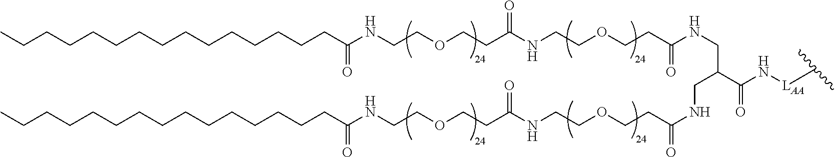

LP 220a

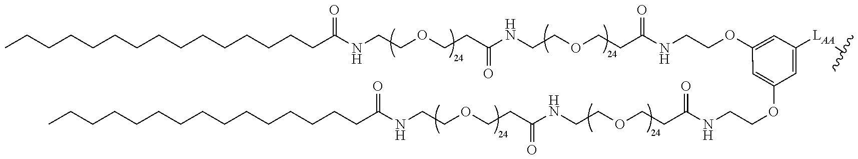

LP 223a

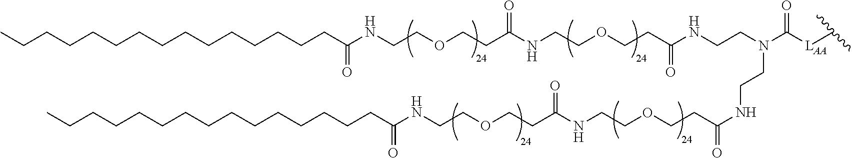

LP 225a

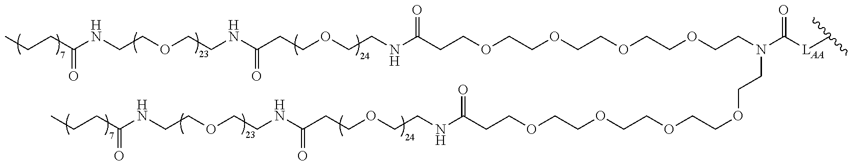

LP 246a

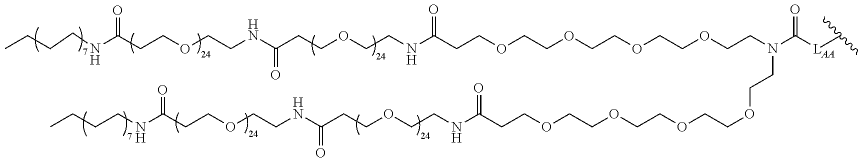

LP 339a

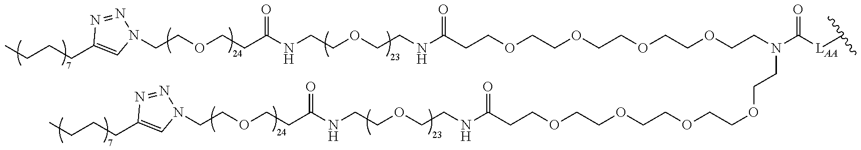

LP 340a

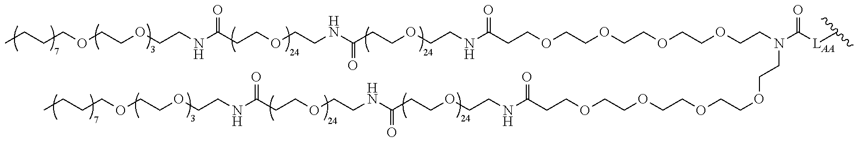

LP 357a

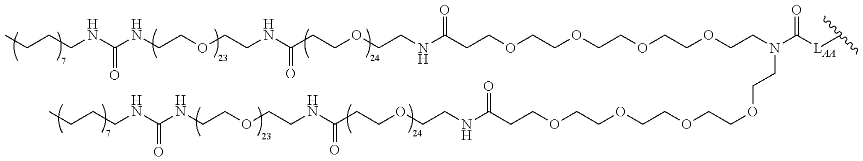

LP 358a

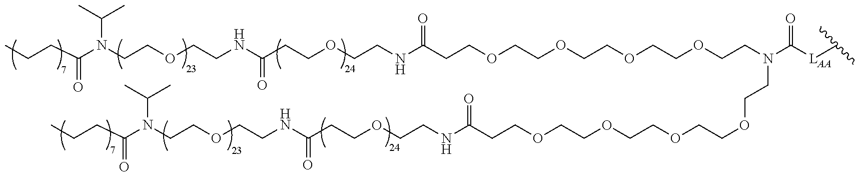

or a pharmaceutically acceptable salt of any of these lipid PK/PD modulators, wherein each L_{AA} is L_{A} as defined in any of the embodiments of the lipid PK/PD modulator of Formula (I), (Ia), (Ib), (Ib1), (Ic), L_{AA} is L_{A2} as defined in any of the embodiments of the lipid PK/PD modulator of Formula (II), L_{AA} is LA₃ as defined in any of the embodiments of the lipid PK/PD modulator of Formula (III), (IIIa), or (IIIb), or L_{AA} is L_{A4} as defined in any of the embodiments of the lipid PK/PD modulator of Formula (IV);
and each [symbol] indicates a point of connection to the RNAi agent.

In some embodiments, each L_{AA} is a bond or bivalent moiety for connecting the RNAi agent to the rest of the lipid PK/PD modulator; and each [symbol] indicates a point of connection to the RNAi agent.

In another aspect of the invention, the RNAi agent may be conjugated to a lipid PK/PD modulator selected from the group consisting of the lipid PK/PD modulators identified in Table 20.

TABLE 20
Example lipid PK/PD modulators of the present invention (compound
number appears before structure).
LP 5a

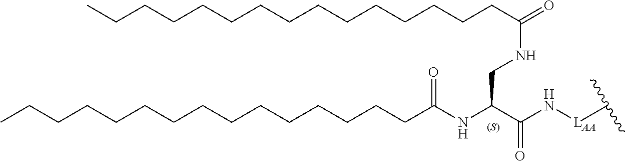

LP 33a

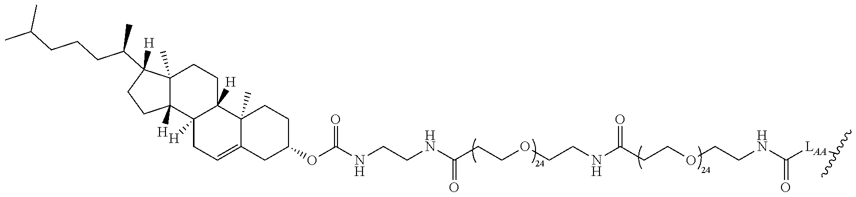

or a pharmaceutically acceptable salt of any of these lipid PK/PD modulator s, wherein each L_{AA} is L_{A} as defined in any of the embodiments of the lipid PK/PD modulator of Formula (I), (Ia), (Ib), (Ib1), (Ic), L_{AA} is L_{A2} as defined in any of the embodiments of the lipid PK/PD modulator of Formula (II), L_{AA} is L_{A3} as defined in any of the embodiments of the lipid PK/PD modulator of Formula (III), (IIIa), or (IIIb), or L_{AA} is L_{A4} as defined in any of the embodiments of the lipid PK/PD modulator of Formula (IV);
and each [symbol] indicates a point of connection to the RNAi agent.

In some embodiments, each L_{AA} is a bond or bivalent moiety for connecting the RNAi agent to the rest of the lipid PK/PD modulator; and each [symbol] indicates a point of connection to the RNAi agent.

In some embodiments, the RNAi agent may be conjugated to a lipid PK/PD modulator selected from the group consisting of the lipid PK/PD modulators identified in Table 21.

TABLE 21
Example lipid PK/PD modulators of the present invention (compound
number appears before structure).
LP 1b

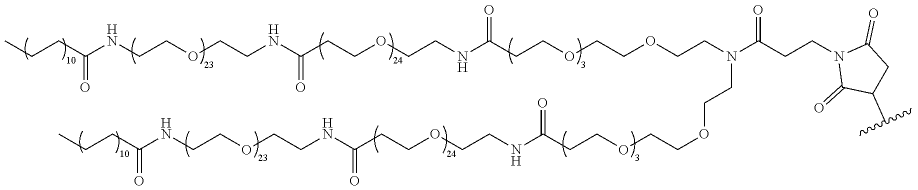

LP 28b

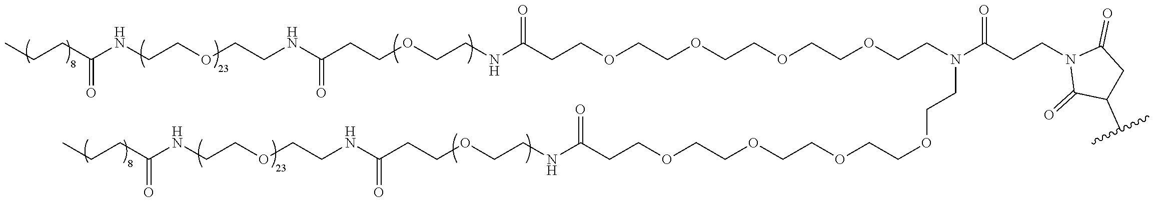

LP 29b

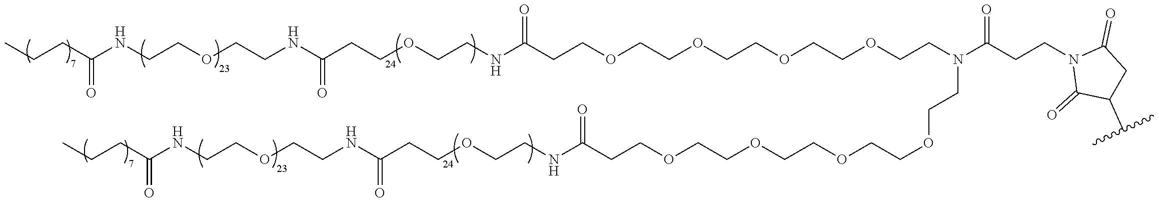

LP 38b

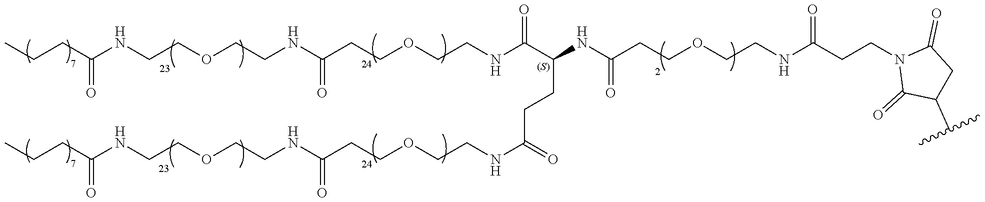

LP 39b

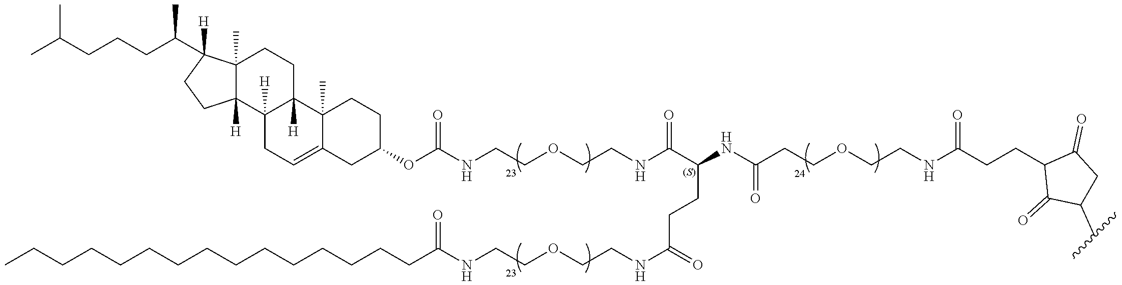

LP 41b

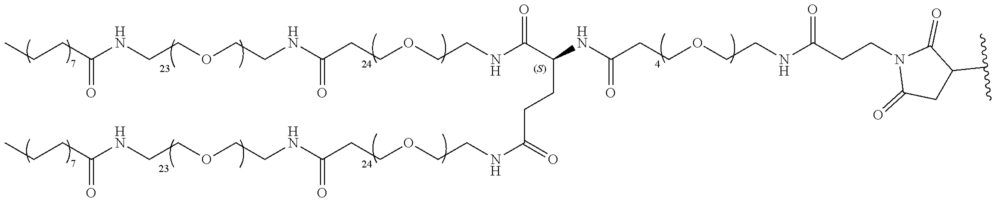

LP 42b

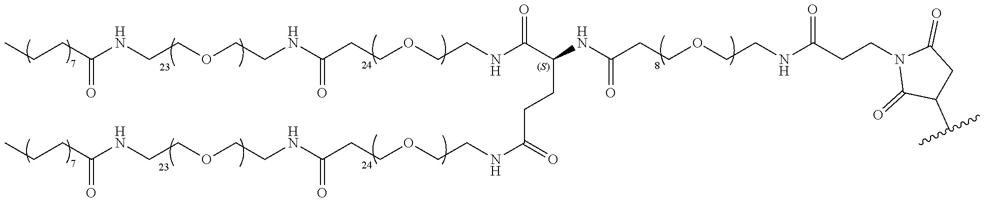

LP 43b

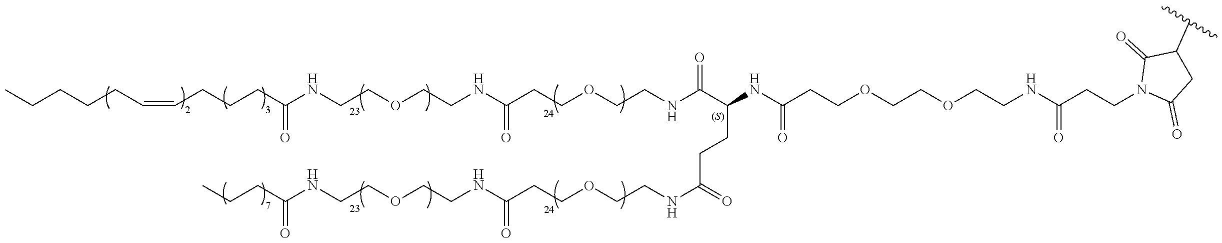

LP 44b

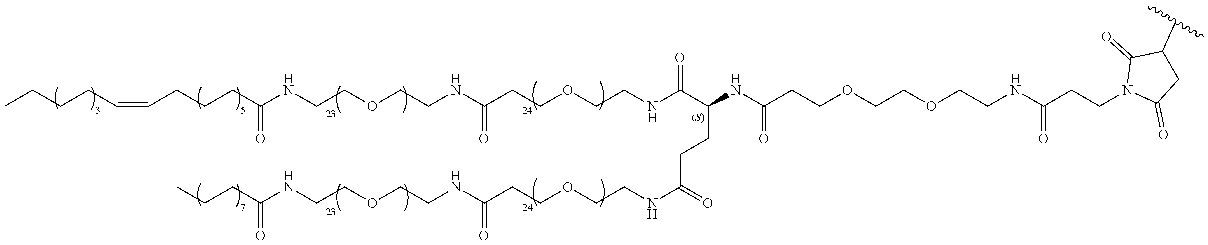

LP 45b

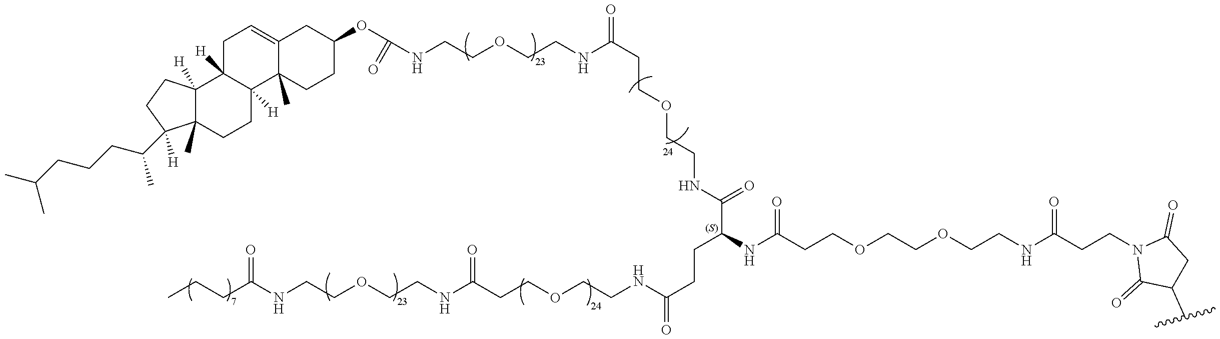

LP 47b

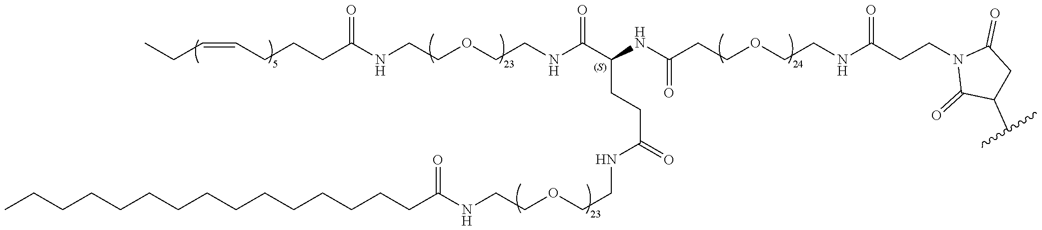

LP 48b

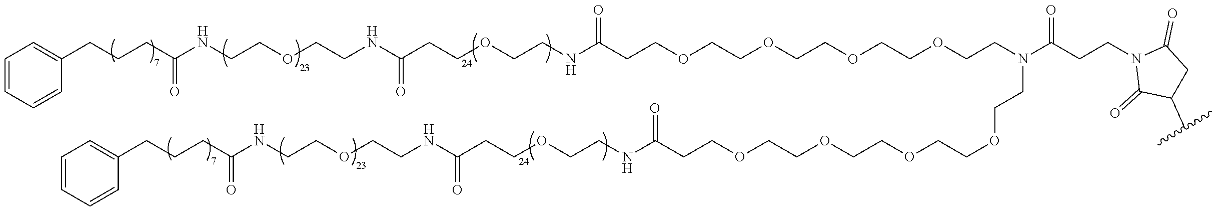

LP 49b

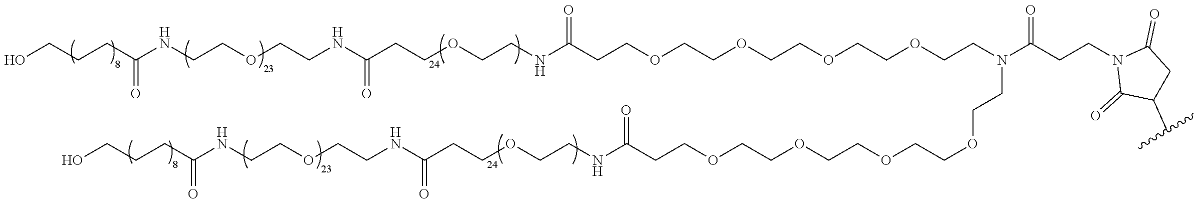

LP 53b

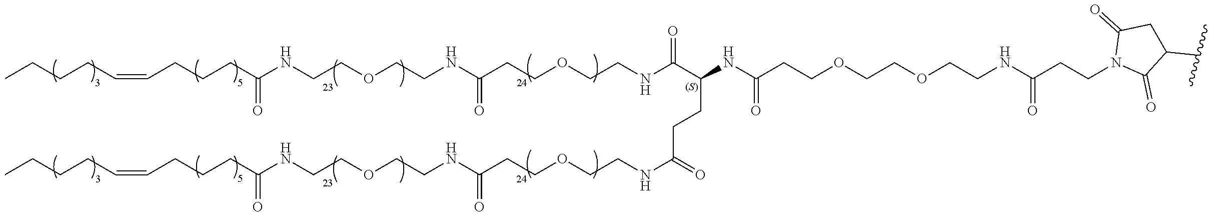

LP 54b

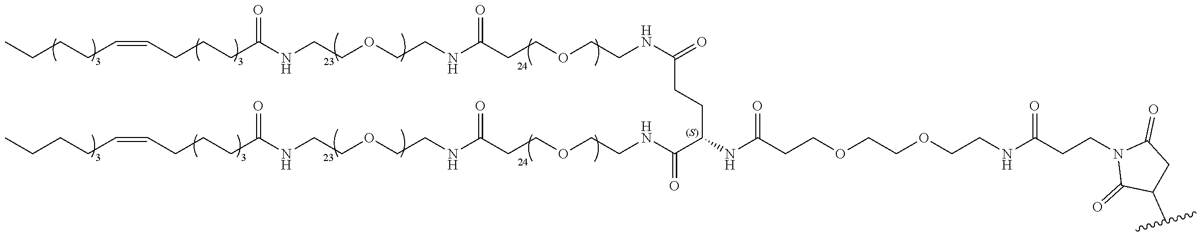

LP 55b

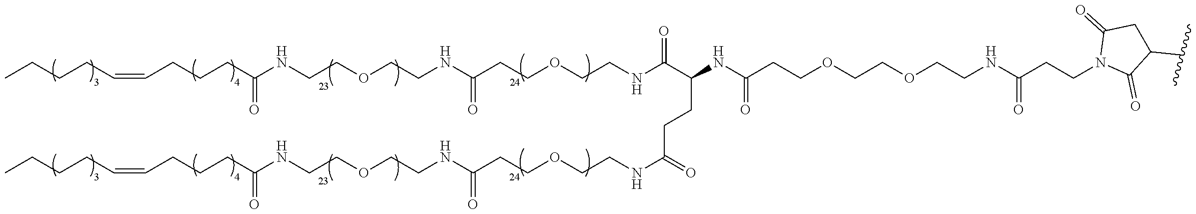

LP 56b

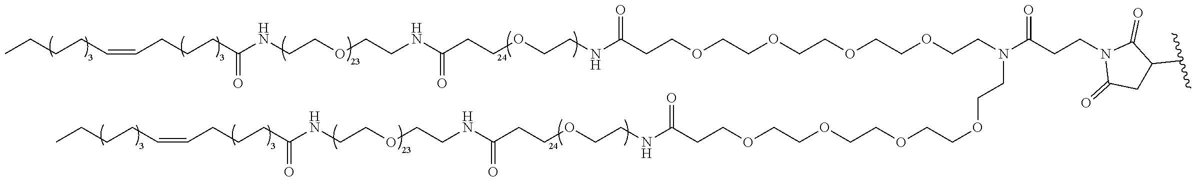

LP 57b

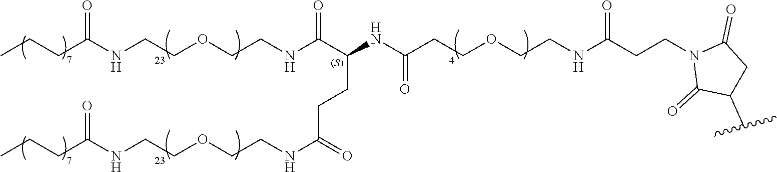

LP 58b

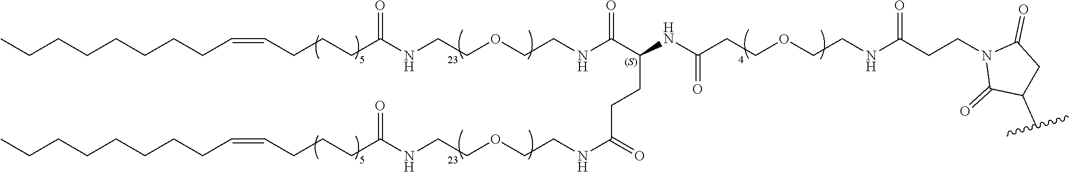

LP 59b

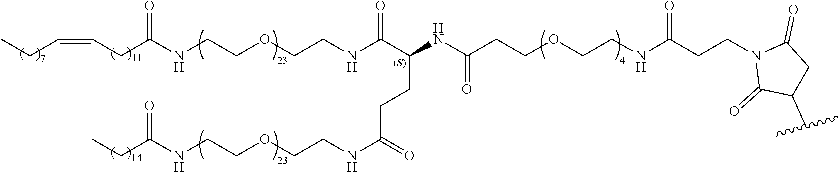

LP 60b

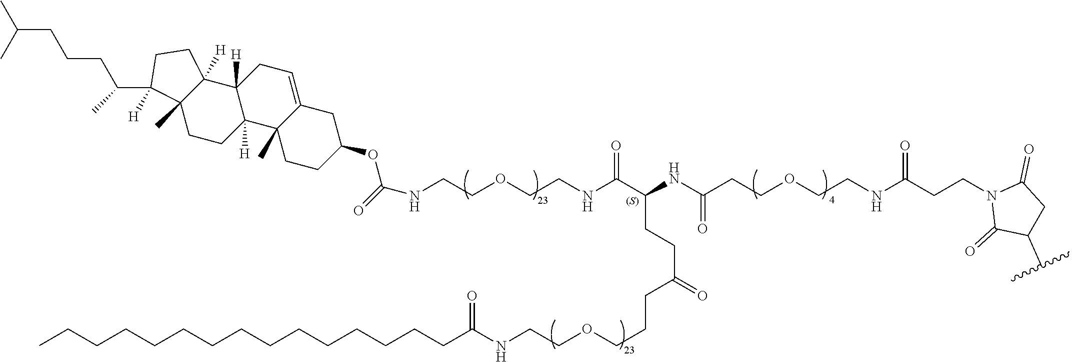

LP 61b

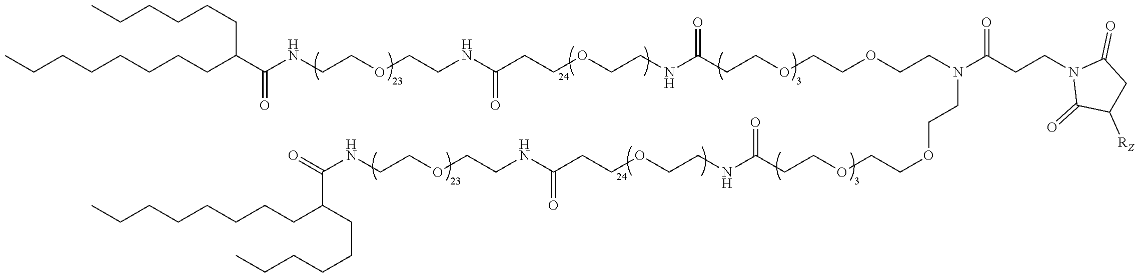

LP 62b

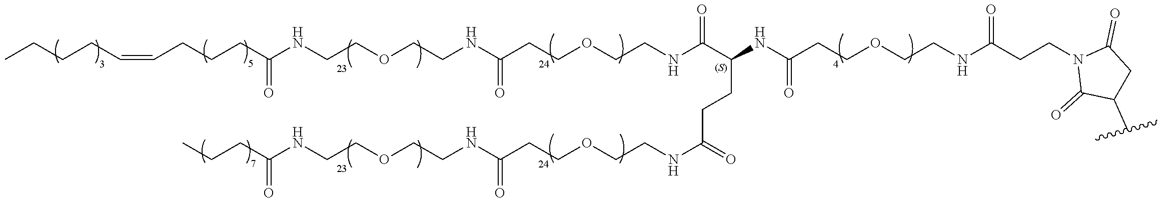

LP 87b

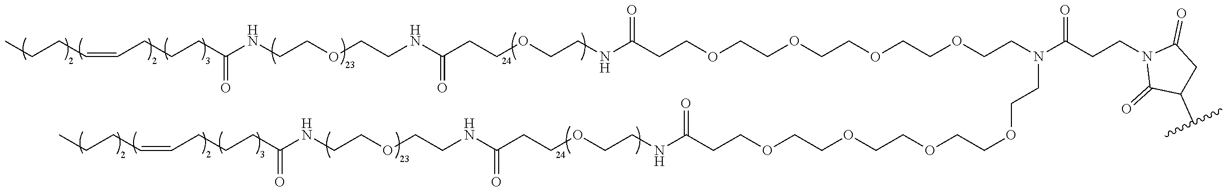

LP 89b

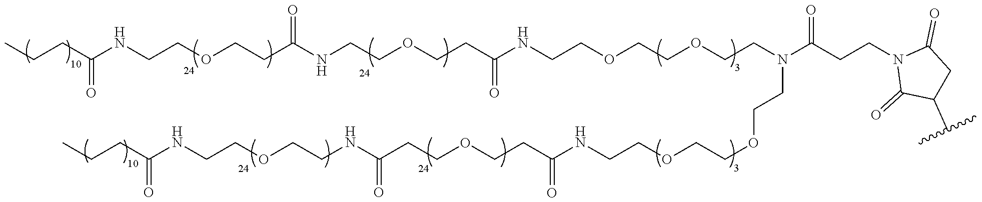

LP 90b

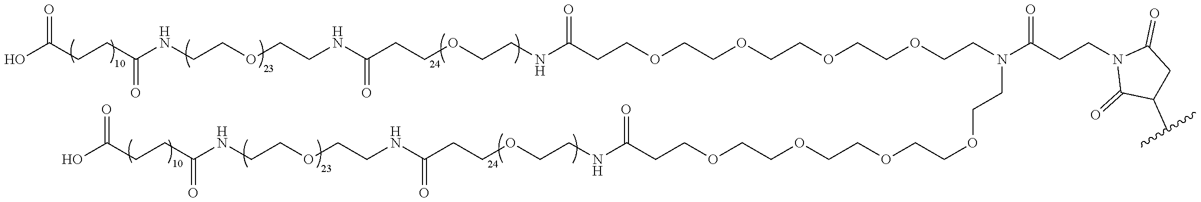

LP 92b

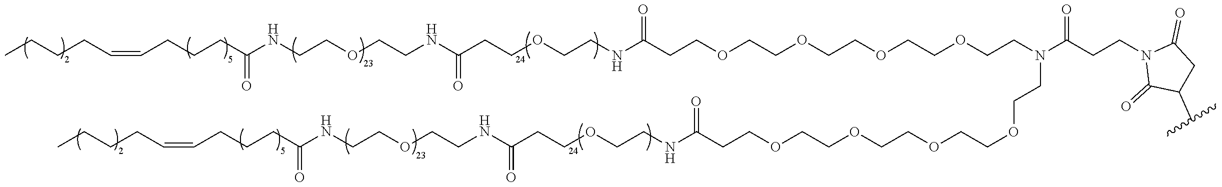

LP 93b

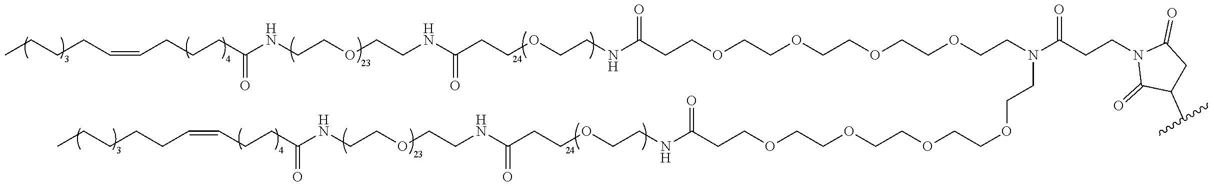

LP 94b

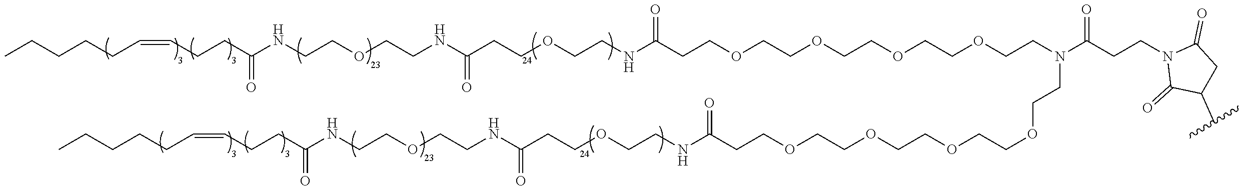

LP 95b

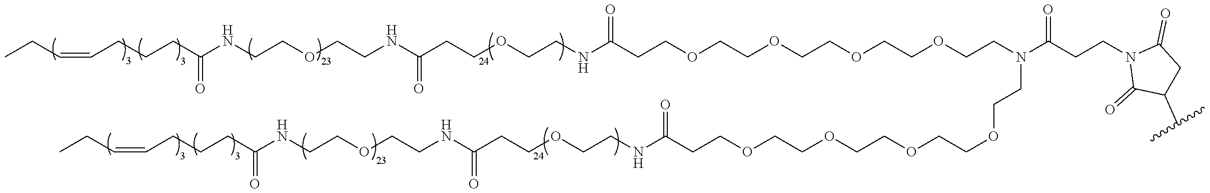

LP 101b

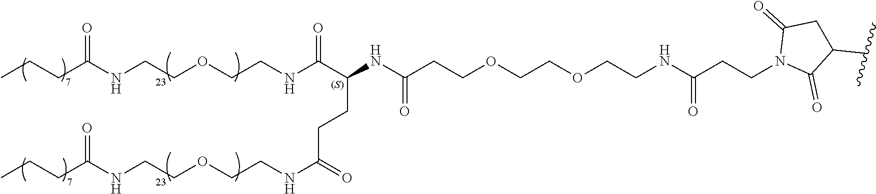

LP 102b

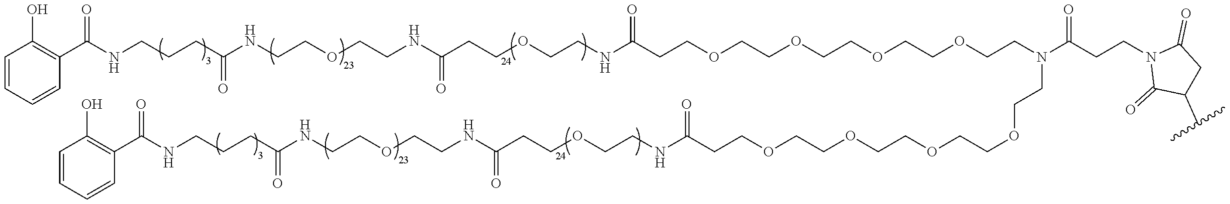

LP 103b

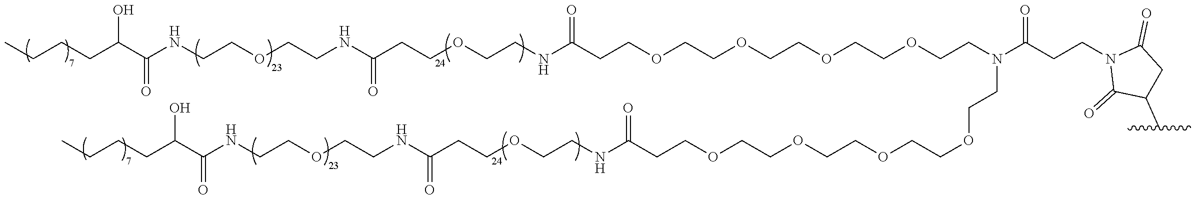

LP 104b

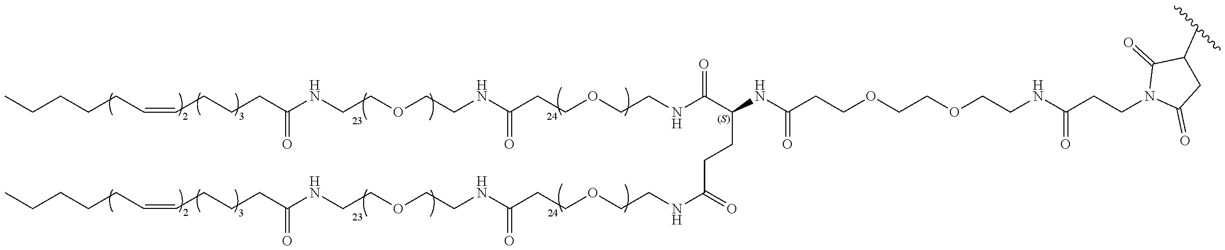

LP 106b

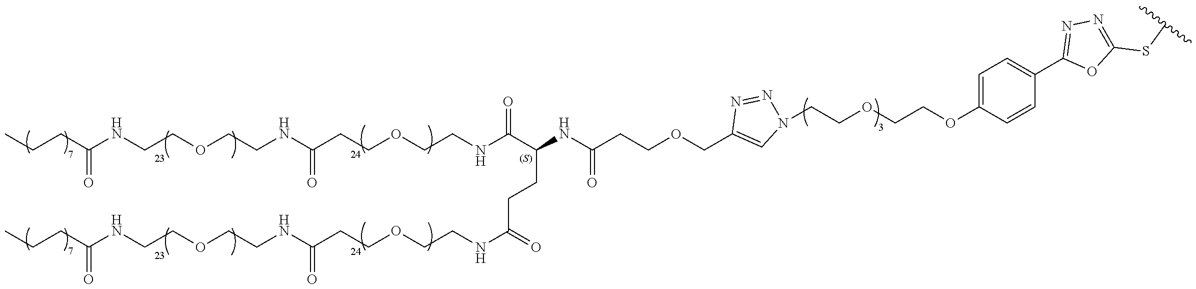

LP 107b

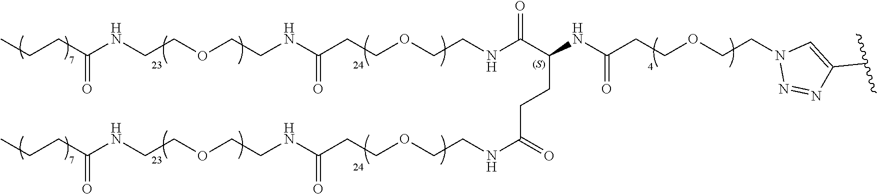

LP 108b

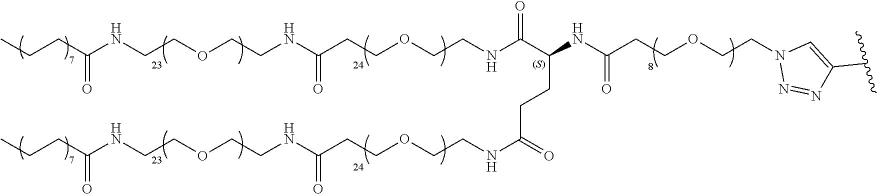

LP 109b

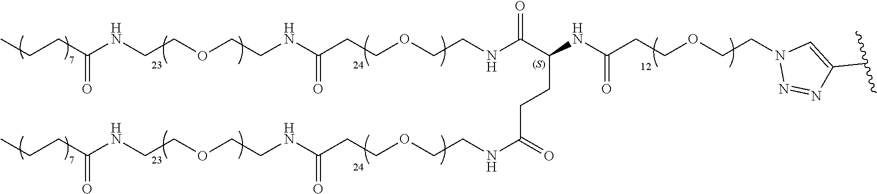

LP 110b

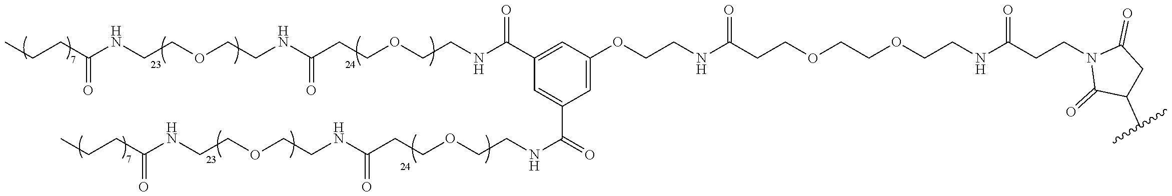

LP 111b

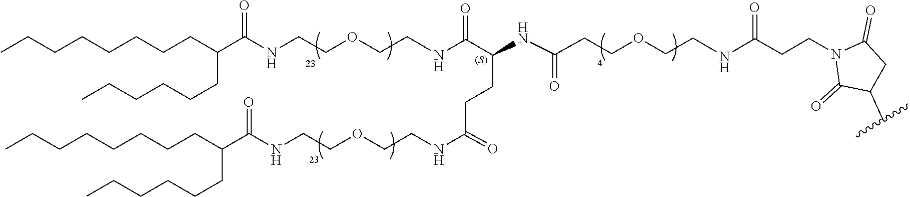

LP 124b

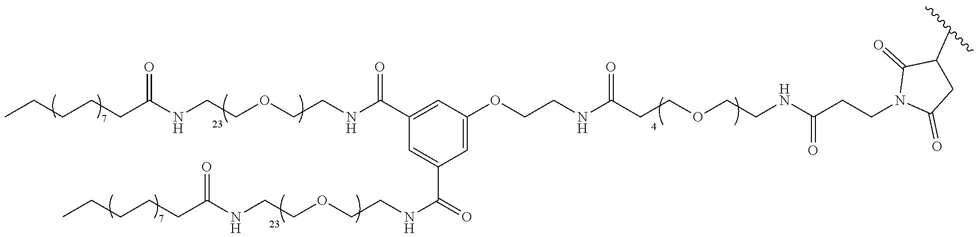

LP 130b

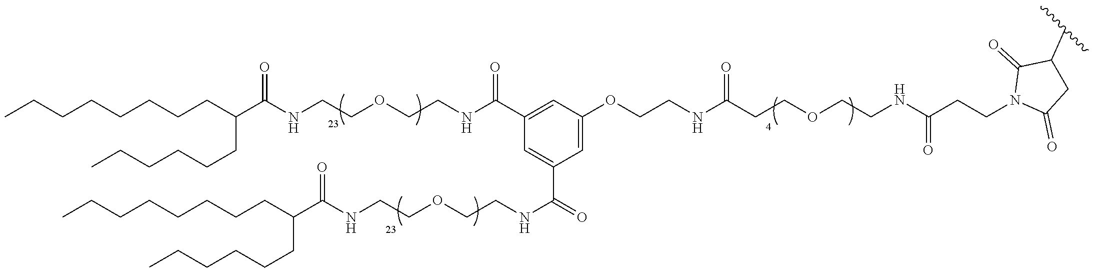

LP 143b

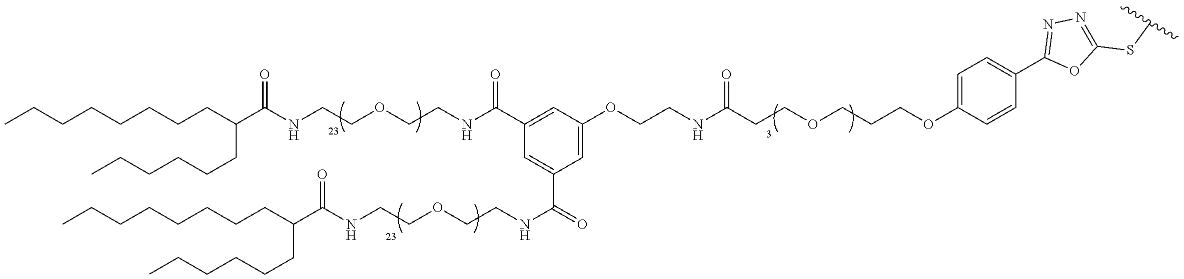

LP 210b

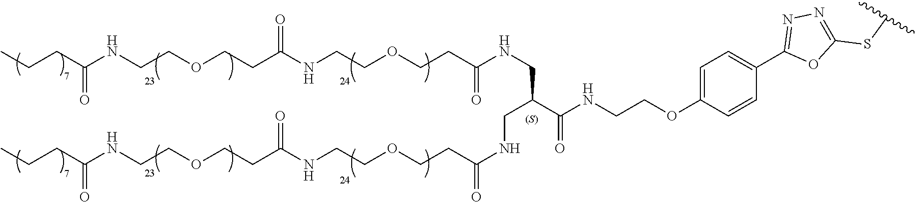

LP 217b

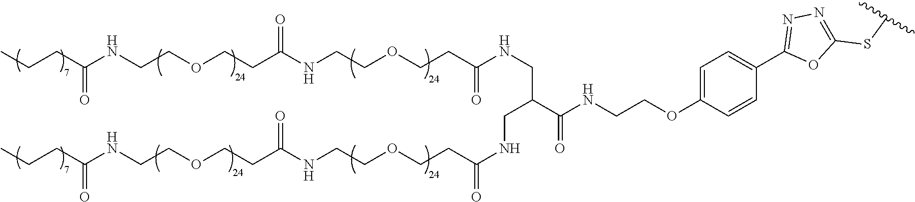

LP 220b

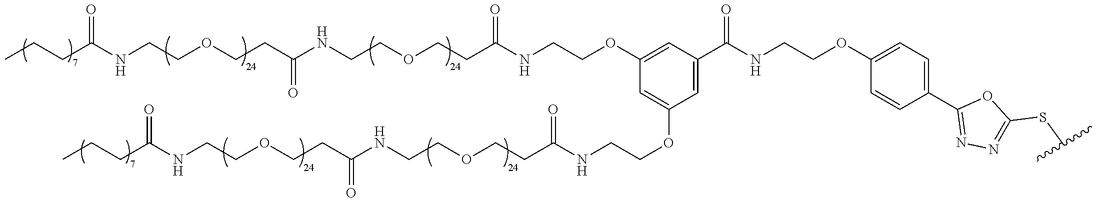

LP 221b

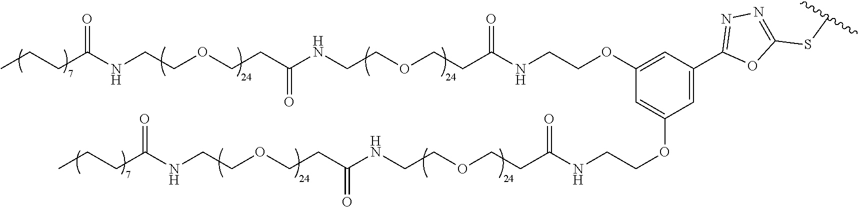

LP 223b

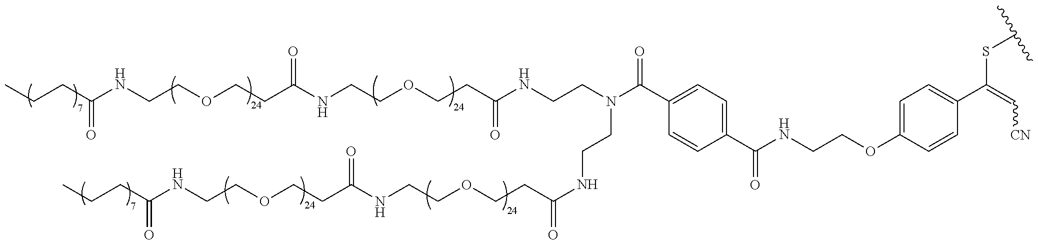

LP 224b

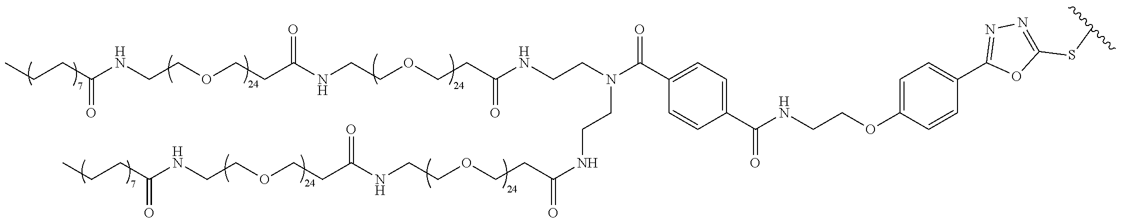

LP 225b

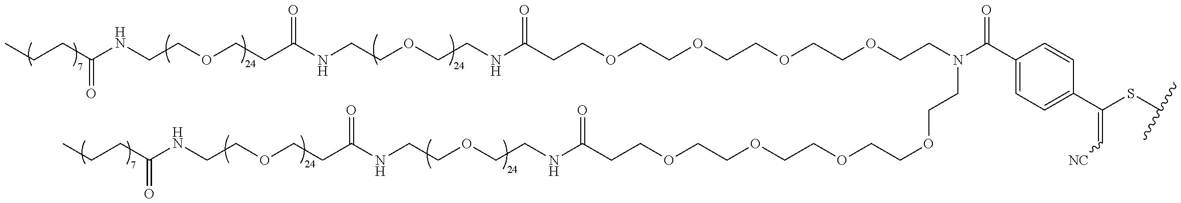

LP 226b

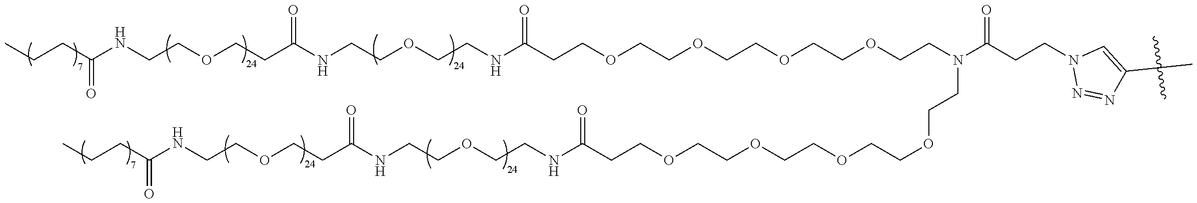

LP 238b

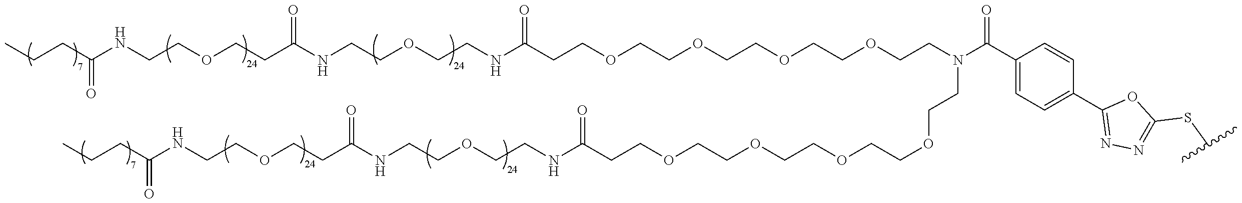

LP 240b

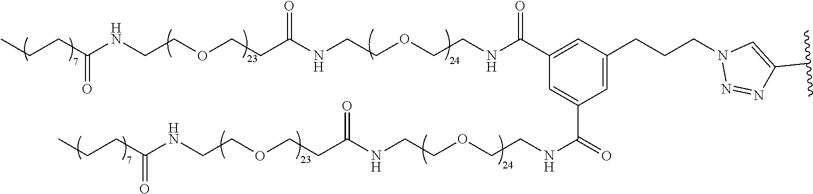

LP 246b

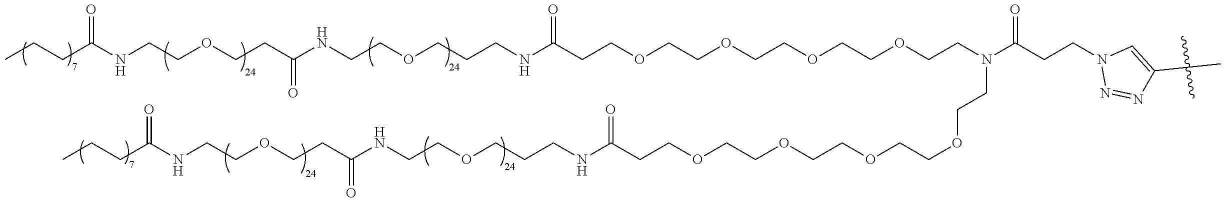

LP 247b

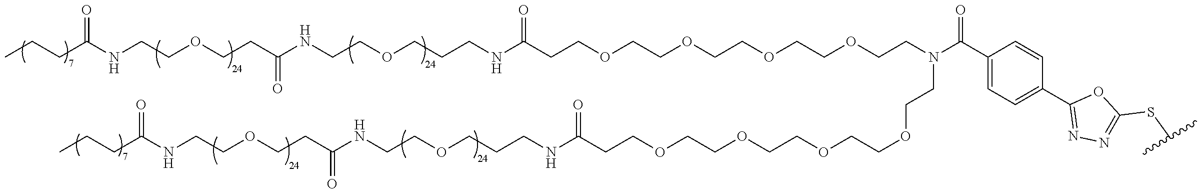

LP 339b

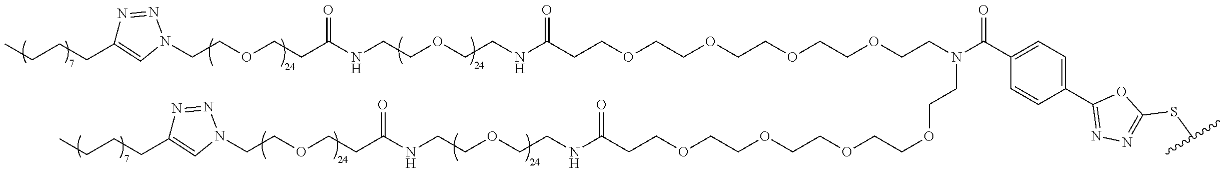

LP 340b

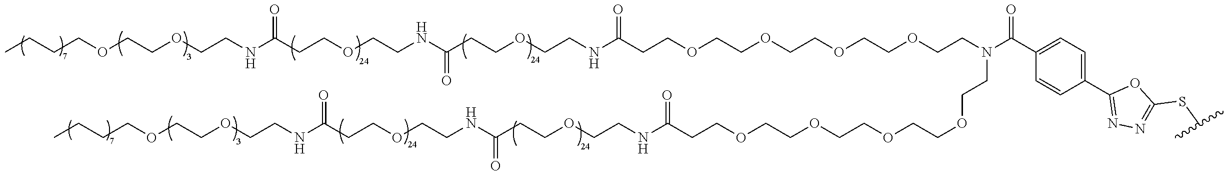

LP 357b

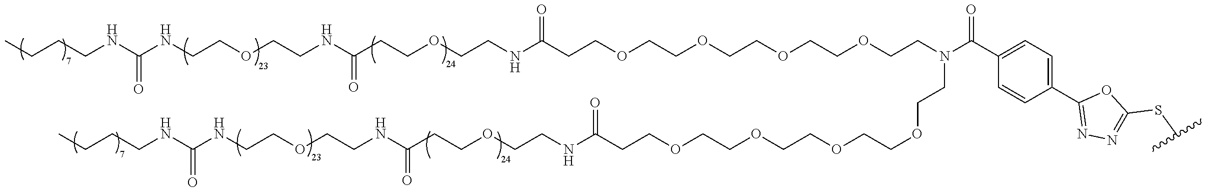

LP 358b

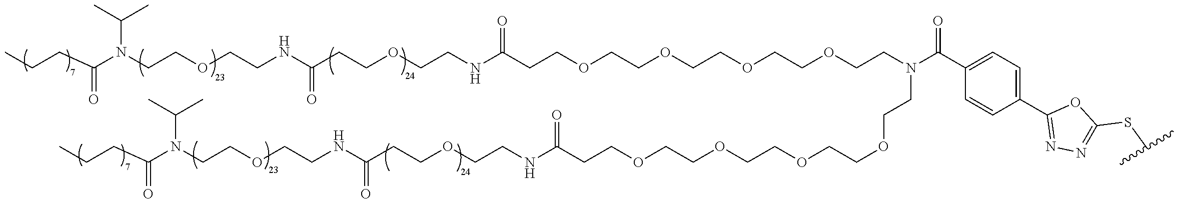

or a pharmaceutically acceptable salt of any of these lipid PK/PD modulators, wherein each [symbol] indicates a point of connection to the RNAi agent.

In another aspect of the invention, the RNAi agent may be conjugated to a lipid PK/PD modulator selected from the group consisting of the lipid PK/PD modulators identified in Table 22.

TABLE 22
Example lipid PK/PD modulators of the present invention (compound
number appears before structure).
LP 5b

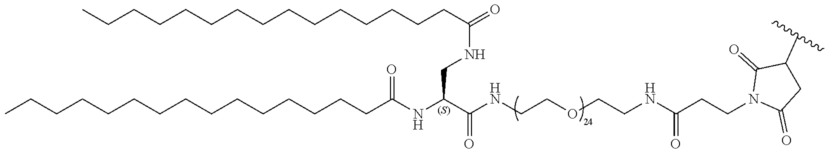

LP 33b

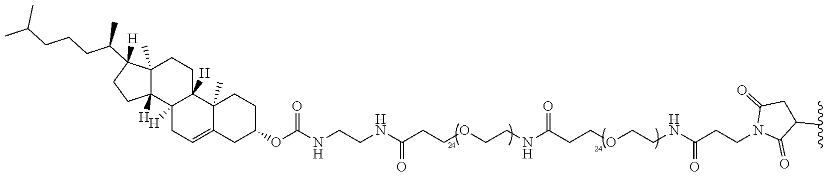

or a pharmaceutically acceptable salt of any of these lipid PK/PD modulators, wherein each [symbol] indicates a point of connection to the RNAi agent.

In some embodiments, the lipid PK/PD modulator precursor suitable for linking to the RNAi agent may be a lipid PK/PD modulator precursor of Formula (V):

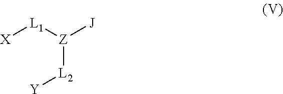

or a pharmaceutically acceptable salt thereof, wherein Z, L₁, L₂, X, and Y are as defined for any embodiments of the lipid PK/PD modulator of Formula (I), (Ia), (Ib), (Ib1), or (Ic); J is L_{A5}-R_{X}; L_{A5} is a bond or a bivalent moiety connecting Rx to Z: and Rx is a reactive moiety for conjugation with the RNAi agent.

In some embodiments, J is L_{A5}-R_{X}; L_{A5} is a bond or a bivalent moiety connecting Rx to Z; Rx is a reactive moiety for conjugation with the RNAi agent; Z is CH, phenyl, or N; L₁ and L₂ are each independently linkers comprising at least about 5 PEG units; and X and Y are each independently lipids comprising from about 10 to about 50 carbon atoms.

In some embodiments, L_{A5} is L_{A} as defined in any embodiments of the lipid PK/PD modulator of Formula (I), (Ia), (Ib), (Ib1), or (Ic). In some embodiments, L_{A5} is selected from the group consisting of the moieties identified in Table 23.

TABLE 23
Example LA5 moieties of the present invention.

| Name | Structure |
|---|---|
| Tether 1-5 | 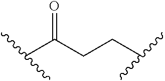 [SMILES: *CCC(*)=O] |
| Tether 2-5 | 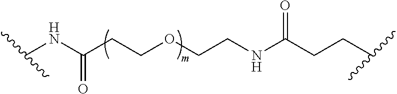 |
| Tether 3-5 | 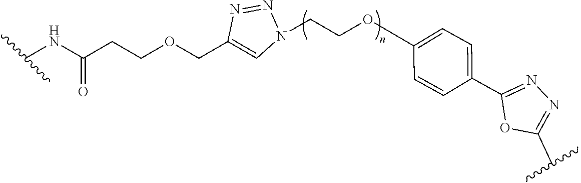 |
| Tether 4-5 | 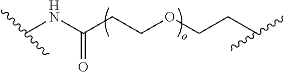 |
| Tether 5-5 | 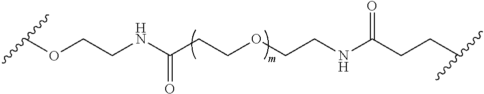 |
| Tether 6-5 | 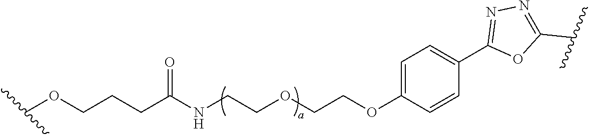 |
| Tether 7-5 | 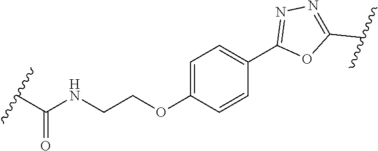 [SMILES: *C(=O)NCCOc1ccc(-c2nnc(*)o2)cc1] |
| Tether 8-5 | 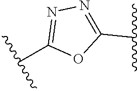 [SMILES: *c1nnc(*)o1] |
| Tether 9-5 | 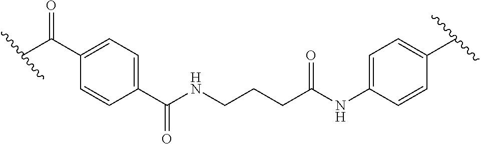 [SMILES: *C(=O)c1ccc(C(=O)NCCCC(=O)Nc2ccc(*)cc2)cc1] |
| Tether 10-5 | 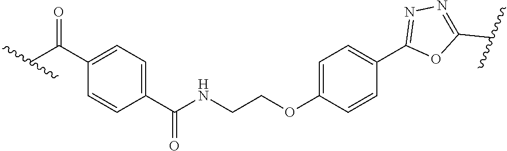 [SMILES: *C(=O)c1ccc(C(=O)NCCOc2ccc(-c3nnc(*)o3)cc2)cc1] |
| Tether 11-5 | 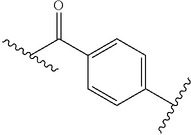 [SMILES: *C(=O)c1ccc(*)cc1] |
| Tether 12-5 | 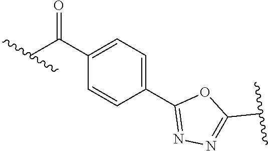 [SMILES: *C(=O)c1ccc(-c2nnc(*)o2)cc1] |
| Tether 13-5 | 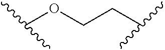 [SMILES: *CCO*] |

wherein each of m, n, o, and a is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and wherein each [symbol] indicates a point of connection to Z or Rx.

In some embodiments, each m is independently 1, 2, 3, 4, 5, 6, 7, 8, 9, 10, 20, 21, 22, 23, or 25; each n is independently 2, 3, 4, or 5; each a is independently 2, 3, or 4; and each o is independently 2, 3, 4, 5, 6, 7, 8, 9, 10, 11, 12, or 13.

In some embodiments, each m is independently 2, 4, 8, or 24. In some embodiments, each n is 4. In some embodiments, each o is independently 4, 8, or 12. In some embodiments, each a is 3.

In some embodiments, Rx is selected from the group consisting of

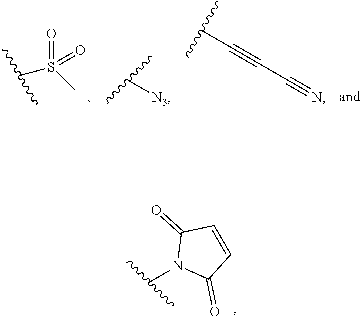

wherein each [symbol] indicates a point of connection to L_{A5}. In some embodiments, Rx is

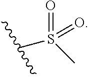 [SMILES: *S(C)(=O)=O]

In some embodiments, Rx is

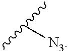 [SMILES: *N=[N+]=[N-]]

In some embodiments, Rx is

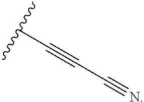 [SMILES: *C#CC#N]

In some embodiments, Rx is

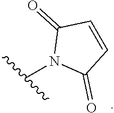 [SMILES: *N1C(=O)C=CC1=O]

In some embodiments, J is selected from the group consisting of the moieties identified in Table 24.

TABLE 24
Example J moieties of the present invention.
Structure

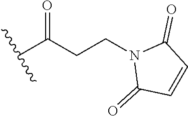 [SMILES: *C(=O)CCN1C(=O)C=CC1=O]

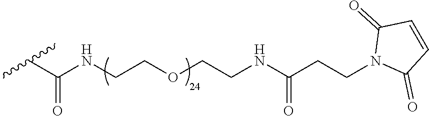

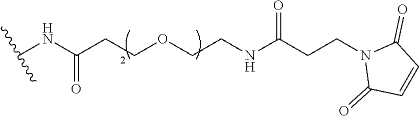

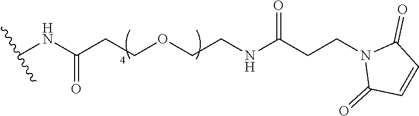

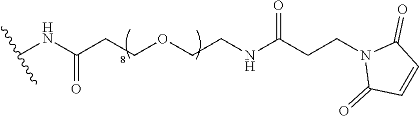

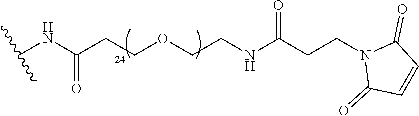

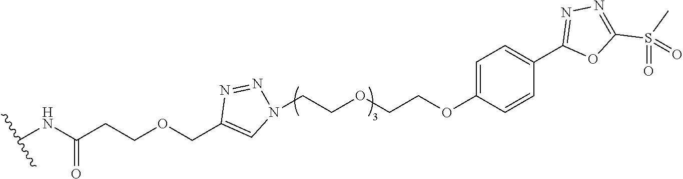

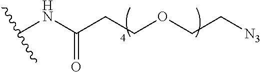

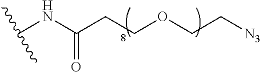

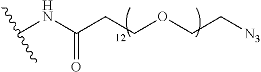

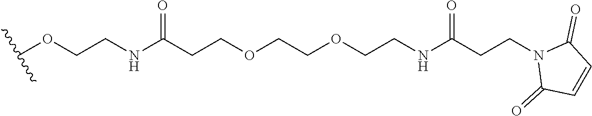 [SMILES: *OCCNC(=O)CCOCCOCCNC(=O)CCN1C(=O)C=CC1=O]

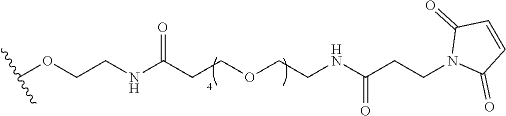

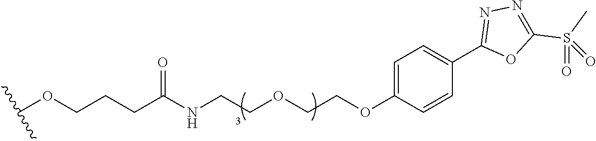

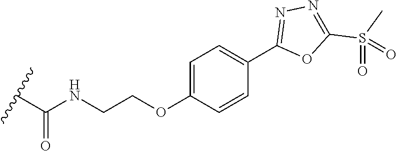 [SMILES: *C(=O)NCCOc1ccc(-c2nnc(S(C)(=O)=O)o2)cc1]

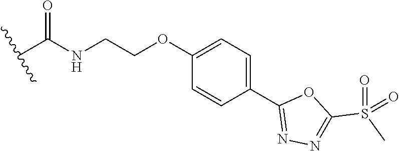 [SMILES: *C(=O)NCCOc1ccc(-c2nnc(S(C)(=O)=O)o2)cc1]

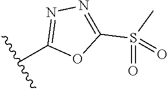 [SMILES: *c1nnc(S(C)(=O)=O)o1]

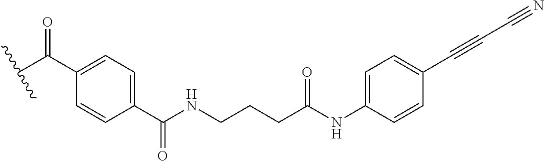 [SMILES: *C(=O)c1ccc(C(=O)NCCCC(=O)Nc2ccc(C#CC#N)cc2)cc1]

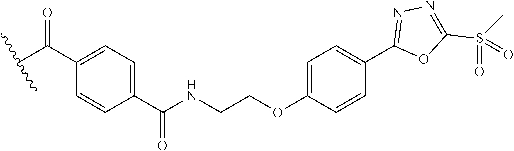 [SMILES: *C(=O)c1ccc(C(=O)NCCOc2ccc(-c3nnc(S(C)(=O)=O)o3)cc2)cc1]

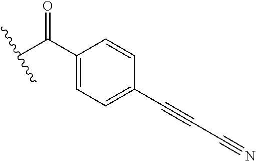 [SMILES: *C(=O)c1ccc(C#CC#N)cc1]

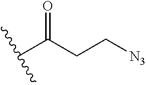 [SMILES: *C(=O)CCN=[N+]=[N-]]

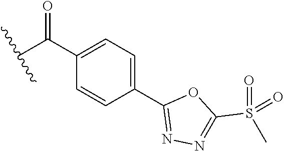 [SMILES: *C(=O)c1ccc(-c2nnc(S(C)(=O)=O)o2)cc1]

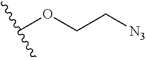 [SMILES: *OCCN=[N+]=[N-]]

wherein each [symbol] indicates a point of connection to Z.

Another aspect of the present invention provides a lipid PK/PD modulator precursor of Formula (Va):

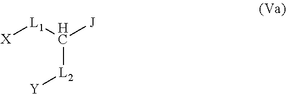

or a pharmaceutically acceptable salt thereof, wherein J, L₁, L₂, X, and Y are as defined in any of the embodiments of the lipid PK/PD modulator precursor of Formula (V).

In some embodiments, X and Y are each independently selected from the group consisting of Lipid 3, Lipid 4, Lipid, 5, Lipid 6, Lipid 7, Lipid 10, Lipid 12, and Lipid 19 as set forth in Table 8, wherein each [symbol] indicates a point of connection to L₁ or L₂.

In some embodiments, each of L₁ and L₂ are independently selected from the group consisting of Linker 2, Linker 3, Linker 4, and Linker 5 as set forth in Table 6, wherein each [symbol] indicates a point of connection to X, Y, or CH of Formula (Va). In some embodiments, each p is 23. In some embodiments, each q is 24.

In some embodiments, L_{A5} is selected from the group consisting of Tether 2-5, Tether 3-5, and Tether 4-5 as set forth in Table 23, wherein each [symbol] indicates a point of connection to Rx or CH of Formula (Va). In some embodiments, m is 2, 4, 8, or 24. In some embodiments, n is 4. In some embodiments, o is 4, 8, or 12.

In some embodiments, each of L₁ and L₂ is independently selected from the group consisting of

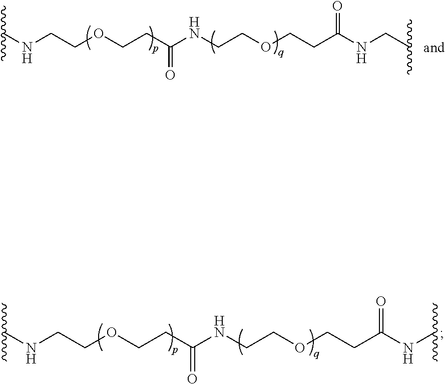

wherein each p is indenpendently 20, 21, 22, 23, 24, or 25; each q is independently 20, 21, 22, 23, 24, or 25; and each [symbol] indicates a point of connection to X, Y, or CH of Formula (Va). In some embodiments, each p is 24. In some embodiments, each q is 24.

In some embodiments, L_{A5} is

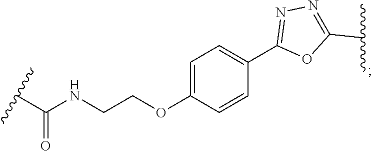 [SMILES: *C(=O)NCCOc1ccc(-c2nnc(*)o2)cc1]

wherein each [symbol] indicates a point of connection to Rx or CH of Formula (Va).

In some embodiments, each of X and Y is

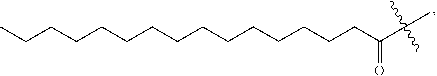 [SMILES: *C(=O)CCCCCCCCCCCCCCC]

wherein [symbol] indicates a point of connection to the L₁ or L₂.

In some embodiments, the lipid PK/PD modulator precursor of Formula (Va) is selected from the group consisting of LP210-p or LP 21′7-p as set forth in Table 25, or a pharmaceutically acceptable salt of any one of these lipid PK/PD modulator precursors.

Another aspect of the present invention provides a lipid PK/PD modulator precursor of Formula (Vb):

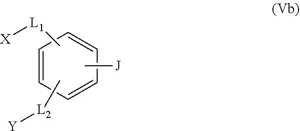

or a pharmaceutically acceptable salt thereof, wherein J, L₁, L₂, X, and Y are as defined in any of the embodiments of the lipid PK/PD modulator precursor of Formula (V) or (Va).

In some embodiments, X and Y are each independently selected from the group consisting of Lipid 3 and Lipid 19 as set forth in Table 8, wherein each [symbol] indicates a point of connection to L₁ or L₂. In some embodiments, X and Y are each Lipid 3. In some embodiments, X and Y are each Lipid 19.

In some embodiments, each of L₁ and L₂ is independently selected from the group consisting of Linker 3, Linker 5, and Linker 9 as set forth in Table 6, wherein each [symbol] indicates a point of connection to X, Y, or the phenyl ring of Formula (Vb). In some embodiments, p is 23 or 24. In some embodiments, q is 24.

In some embodiments, L_{A5} is selected from the group consisting of Tether 5-5, Tether, 6-5, Tether 7-5, Tether 8-5, and Tether 13-5 as set forth in Table 23, wherein each [symbol] indicates a point of connection to Rx or the phenyl ring of Formula (Vb). In some embodiments, m is 2 or 4. In some embodiments, a is 3.

Another aspect of the present invention provides a lipid PK/PD modulator precursor of Formula (Vb1):

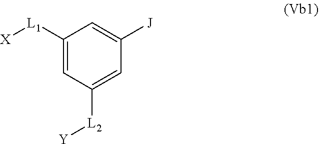

or a pharmaceutically acceptable salt thereof, wherein J, L₁, L₂, X, and Y are as defined in any of the embodiments of the lipid PK/PD modulator precursor of Formula (V), (Va), or (Vb).

Another aspect of the present invention provides a lipid PK/PD modulator precursor of Formula (Vc):

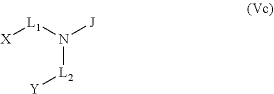

or a pharmaceutically acceptable salt thereof, wherein J, L₁, L₂, X, and Y are as defined in any of the embodiments of the lipid PK/PD modulator precursor of Formula (V), (Va), (Vb), or (Vb1).

In some embodiments, X and Y are each independently selected from the group consisting of Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, and Lipid 24 as set forth in Table 4, wherein each [symbol] indicates a point of connection to L₁ and L₂. In some embodiments, each of X and Y is Lipid 1, Lipid 2, Lipid 3, Lipid 5, Lipid 8, Lipid 9, Lipid 11, Lipid 12, Lipid 14, Lipid 15, Lipid 16, Lipid 17, Lipid 18, Lipid 19, Lipid 20, Lipid 21, Lipid 22, Lipid 23, or Lipid 24.

In some embodiments, each of L₁ and L₂ is independently selected from the group consisting of Linker 1, Linker 6, Linker 10, Linker 11, and Linker 12 as set forth in Table 2, wherein each [symbol] indicates a point of connection to X, Y, or N of Formula (Vc). In some embodiments, p is 23 or 24. In some embodiments, q is 24. In some embodiments, r is 4.

In some embodiments, L_{A5} is selected from the group consisting of Tether 1-5, Tether 9-5, Tether 10-5, Tether 11-5, or Tether 12-5 as set forth in Table 23, wherein each [symbol] indicates a point of connection to the RNAi agent or N of Formula (Vc).

Another aspect of the present invention provides a lipid PK/PD modulator precursor of Formula (Vd):

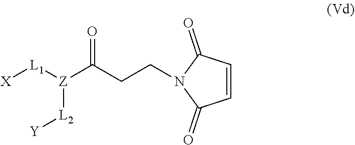

or a pharmaceutically acceptable salt thereof, wherein Z, L₁, L₂, X, and Y are as defined in any of the embodiments of the lipid PK/PD modulator precursor of Formula (V), (Va), (Vb) (Vb1), or (Vc).

Another aspect of the present invention provides a lipid PK/PD modulator precursor of Formula (Ve):

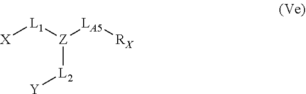

or a pharmaceutically acceptable salt thereof, wherein Z, L₁, L₂, Rx, L_{A5}, X, and Y are as defined in any of the embodiments of the lipid PK/PD modulator precursor of Formula (V), (Va), (Vb) (Vb1), (Vc) or (Vd).

Another aspect of the present invention provides a lipid PK/PD modulator precursor of Formula (Ve1):

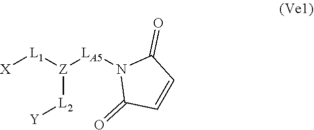

or a pharmaceutically acceptable salt thereof, wherein Z, L₁, L₂, L_{A5}, X, and Y are as defined in any of the embodiments of the lipid PK/PD modulator precursor of Formula (V), (Va), (Vb) (Vb1), (Vc), (Vd), or (Ve).

Another aspect of the present invention provides a lipid PK/PD modulator precursor of Formula (Ve2):

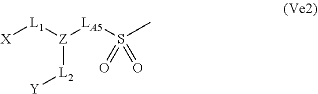

or a pharmaceutically acceptable salt thereof, wherein Z, L₁, L₂, L_{A5}, X, and Y are as defined in any of the embodiments of the lipid PK/PD modulator precursor of Formula (V), (Va), (Vb) (Vb1), (Vc), (Vd), (Ve), or (Ve1).

Another aspect of the present invention provides a lipid PK/PD modulator precursor of Formula (Ve3):

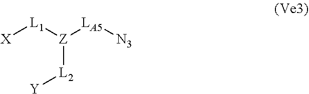

or a pharmaceutically acceptable salt thereof, wherein Z, L₁, L₂, L_{A5}, X, and Y are as defined in any of the embodiments of the lipid PK/PD modulator precursor of Formula (V), (Va), (Vb) (Vb1), (Vc), (Vd), (Ve), (Ve1), or (Ve2).

Another aspect of the present invention provides a lipid PK/PD modulator precursor of Formula (Ve4):

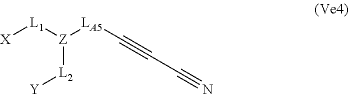

or a pharmaceutically acceptable salt thereof, wherein Z, L₁, L₂, L_{A5}, X, and Y are as defined in any of the embodiments of the lipid PK/PD modulator precursor of Formula (V), (Va), (Vb) (Vb1), (Vc), (Vd), (Ve), (Ve1), (Ve2), or (Ve3).

In some embodiments, the lipid PK/PD modulator precursor may be selected from the group consisting of the lipid PK/PD modulator precursors identified in Table 25.

TABLE 25
Example lipid PK/PD modulator precursors of the present invention
(compound number appears before structure).

| | |
|---|---|
| 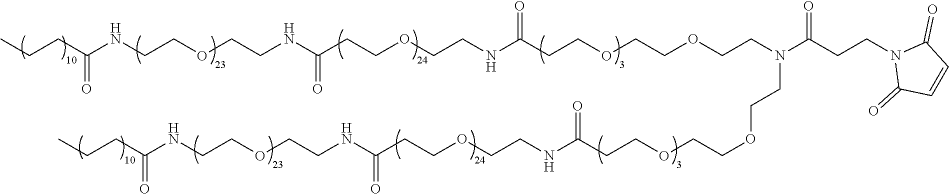 | LP1-p |
| 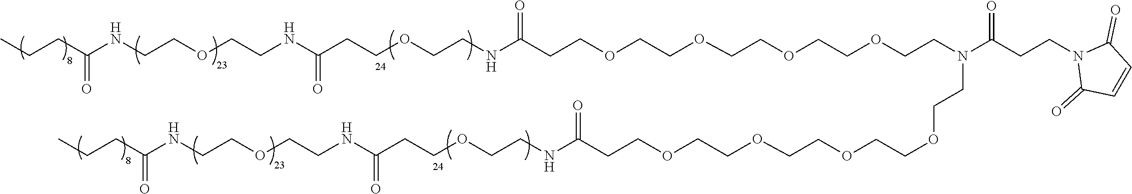 | LP28-p |
| 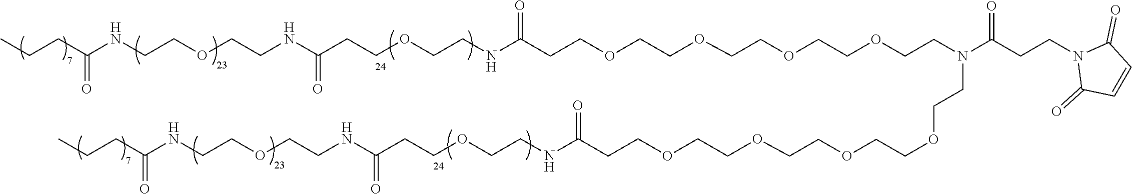 | LP29-p |
| 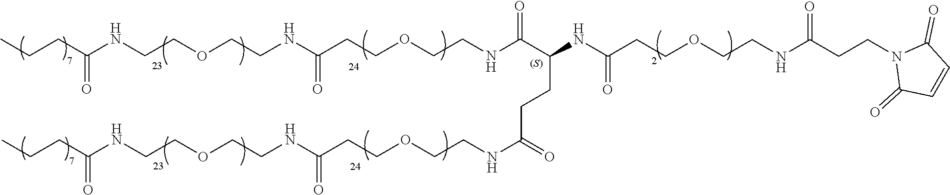 | LP38-p |
| 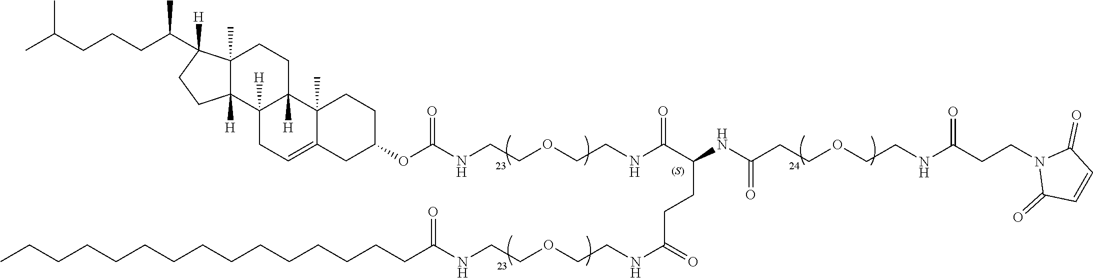 | LP39-p |
| 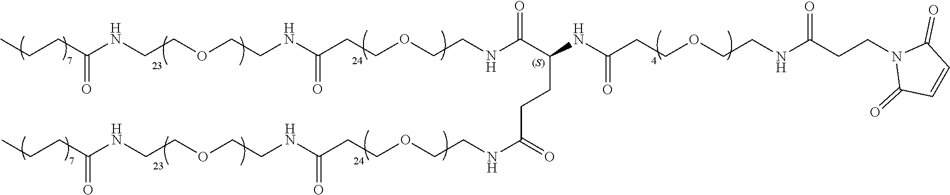 | LP41-p |
| 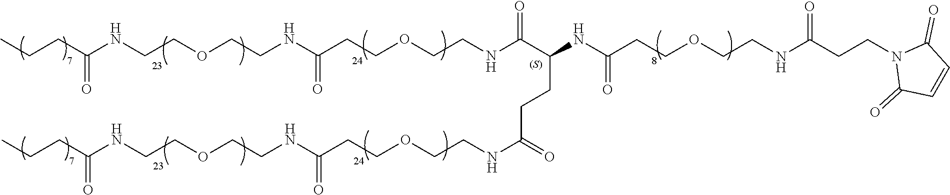 | LP42-p |
| 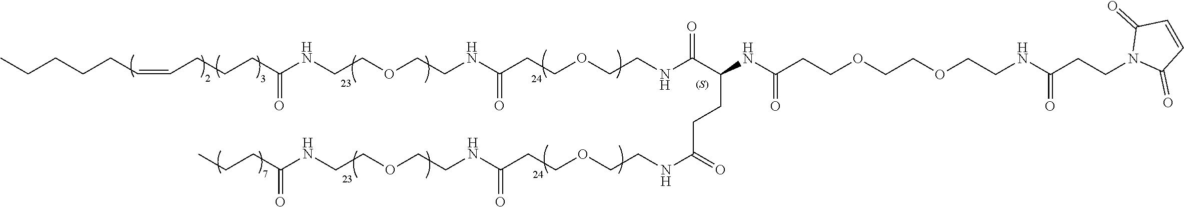 | LP43-p |
| 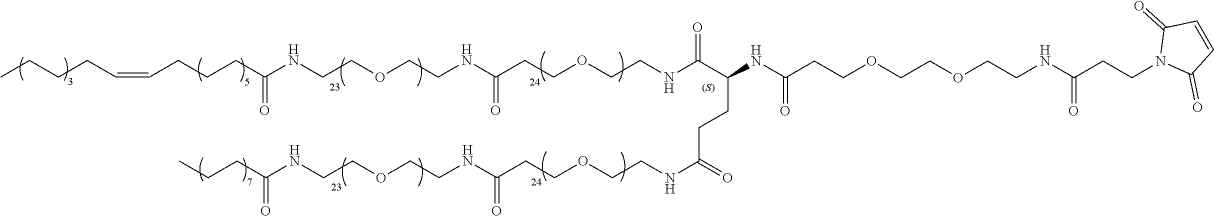 | LP44-p |
| 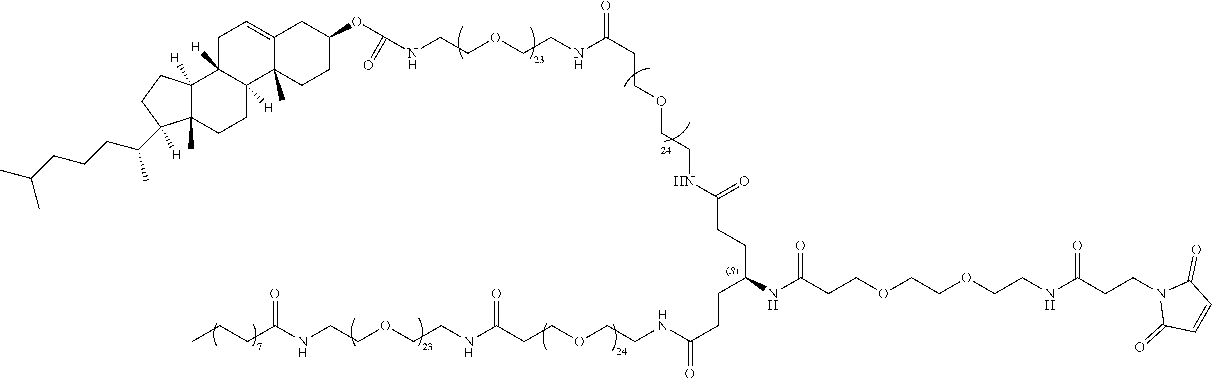 | LP45-p |
| 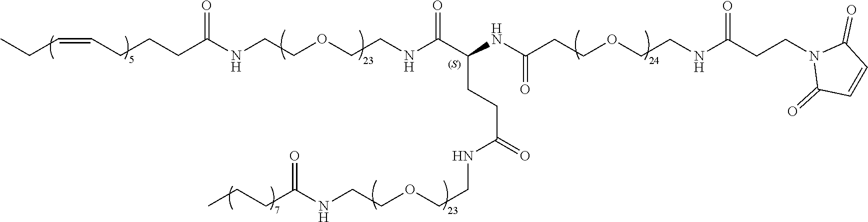 | LP47-p |
| 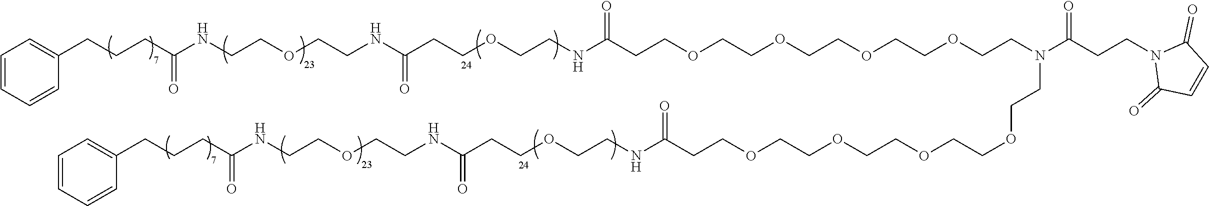 | LP48-p |
| 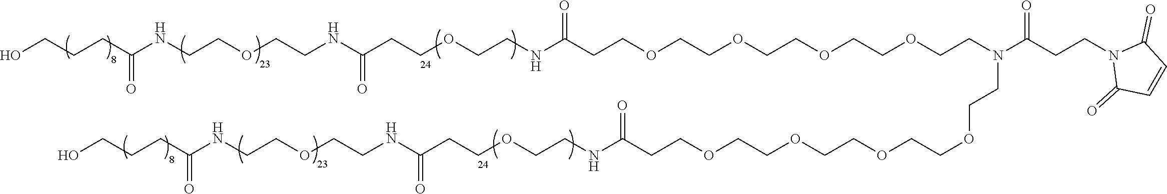 | LP49-p |
| 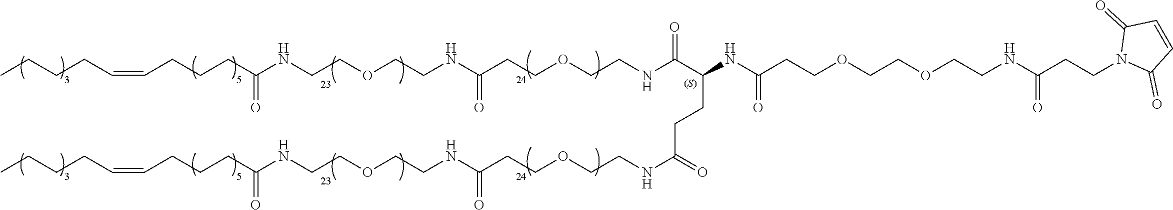 | LP53-p |
| 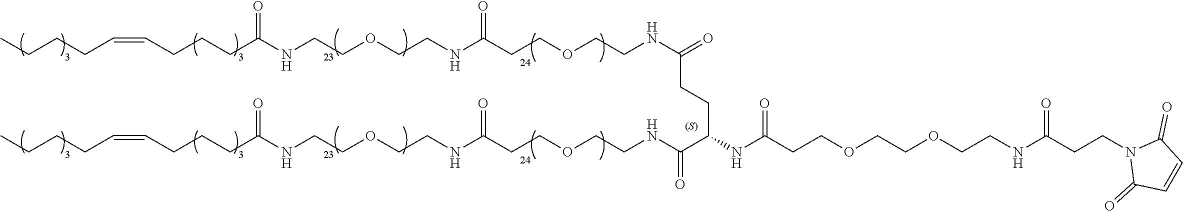 | LP54-p |
| 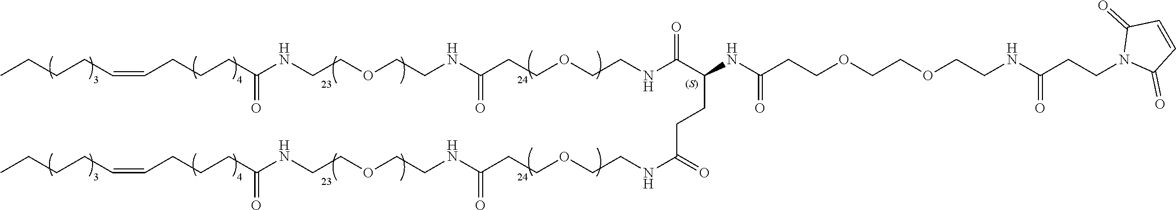 | LP55-p |
| 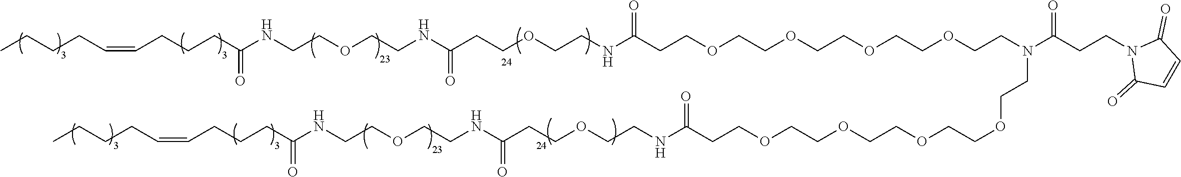 | LP56-p |
| 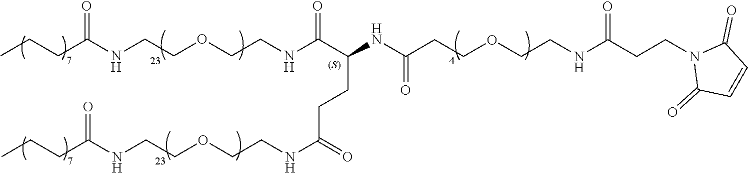 | LP57-p |
| 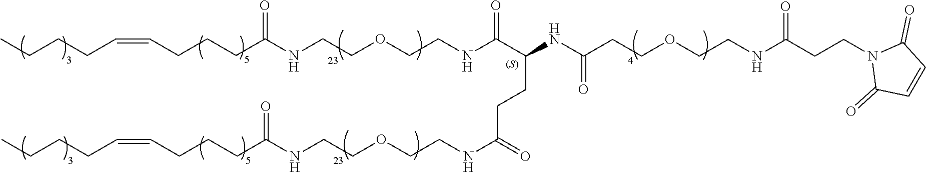 | LP58-p |
| 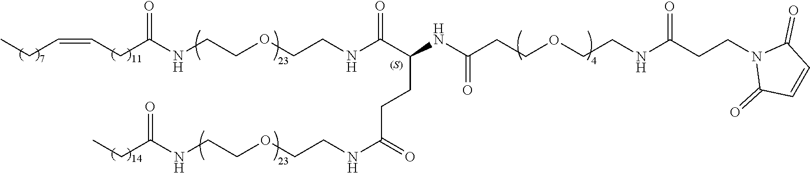 | LP59-p |
| 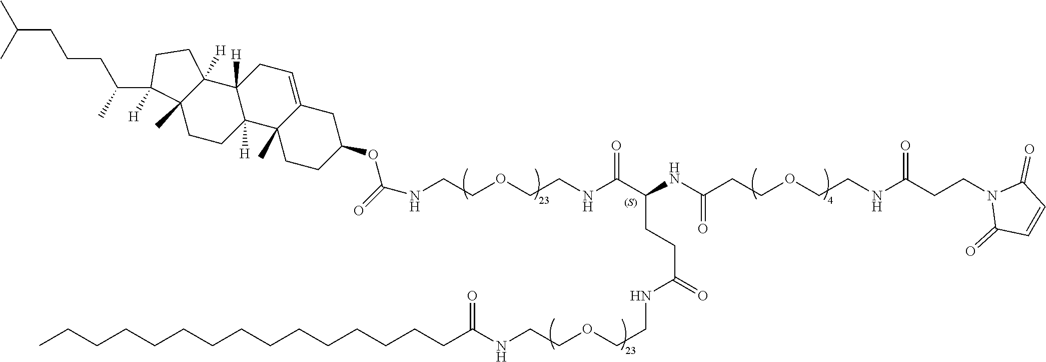 | LP60-p |
| 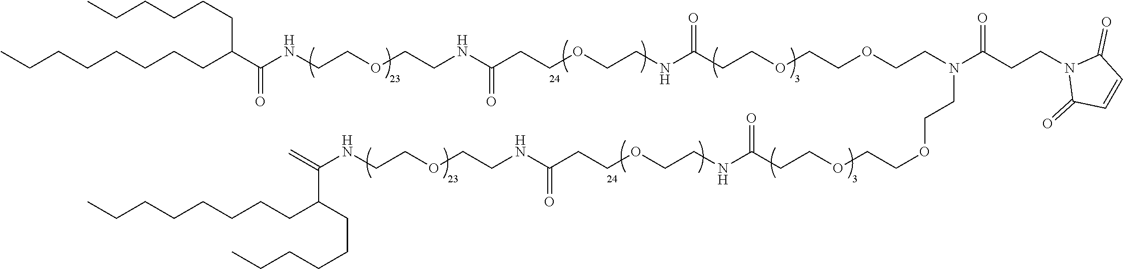 | LP61-p |
| 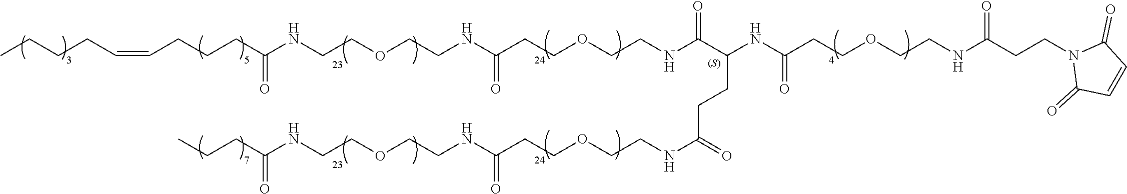 | LP62-p |
| 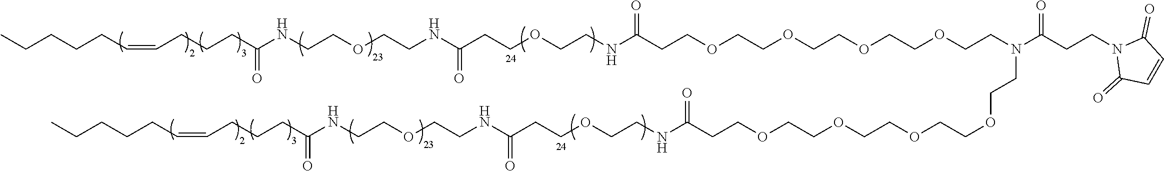 | LP87-p |
| 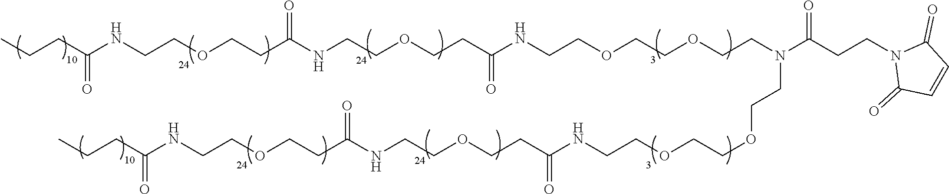 | LP89-p |
| 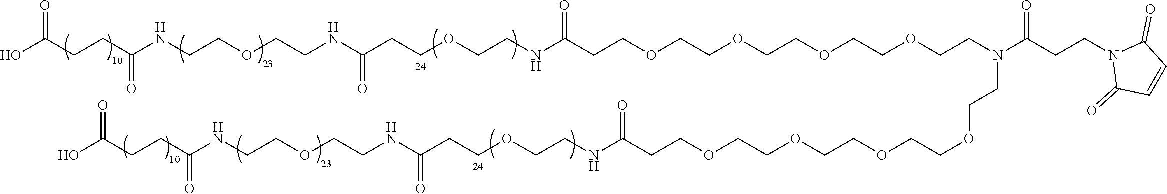 | LP90-p |
| 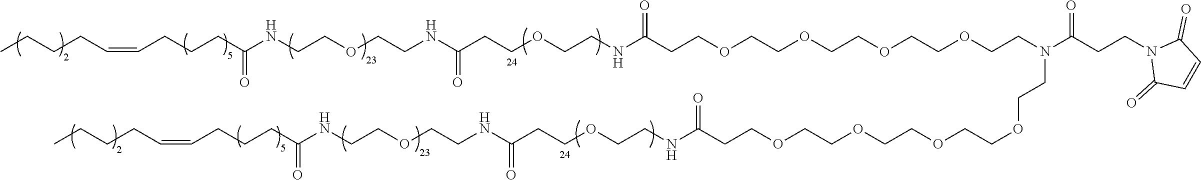 | LP92-p |
| 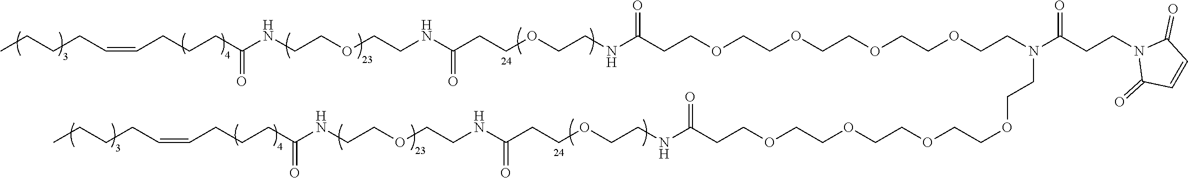 | LP93-p |
| 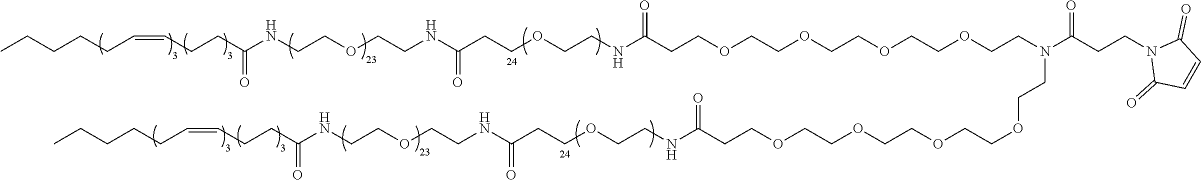 | LP94-p |
| 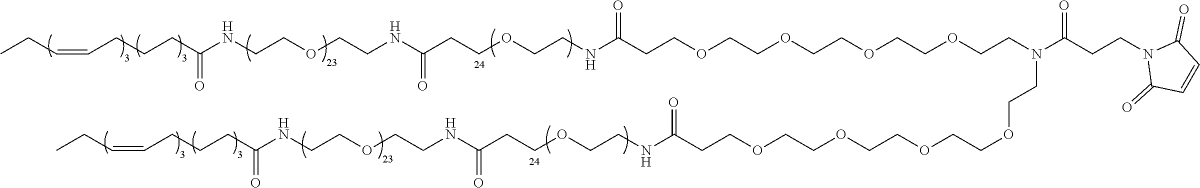 | LP95-p |
| 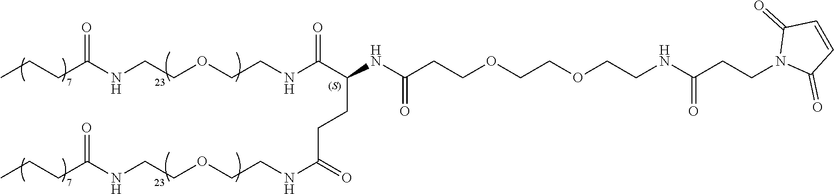 | LP101-p |
| 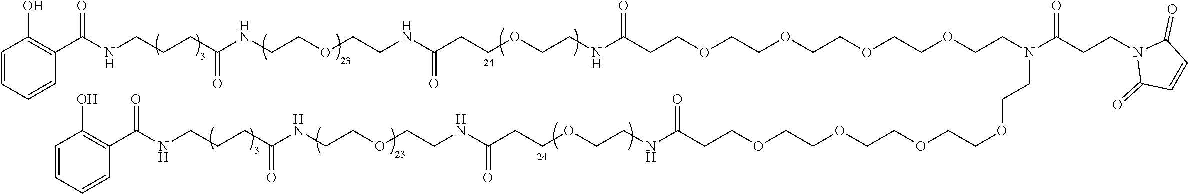 | LP102-p |
| 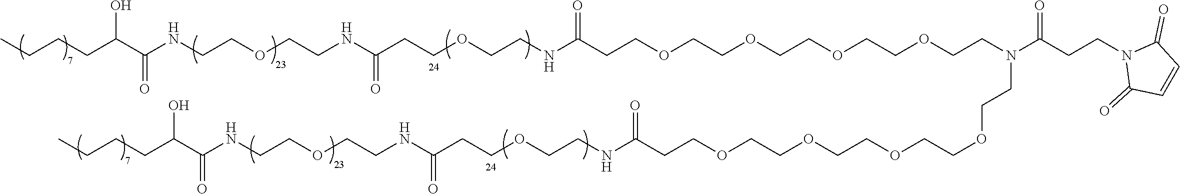 | LP103-p |
| 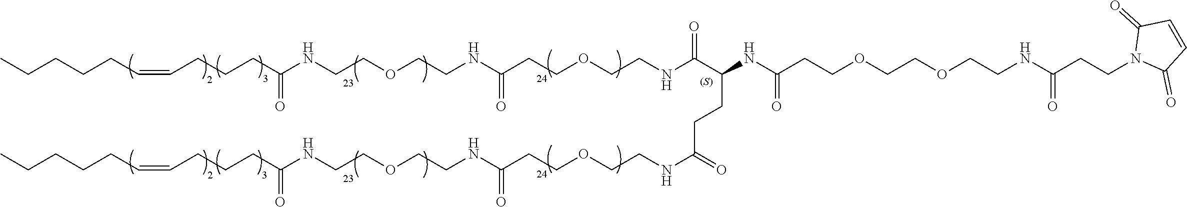 | LP104-p |
| 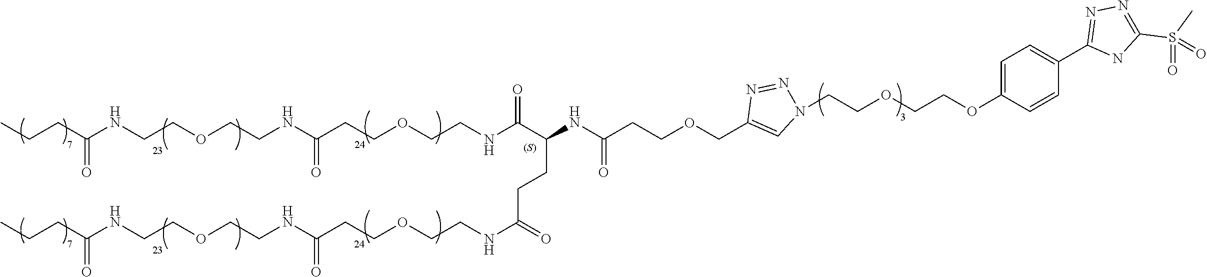 | LP106-p |
| 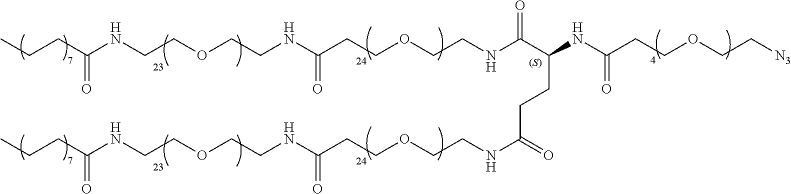 | LP107-p |
| 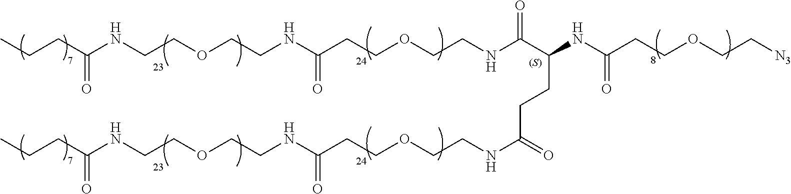 | LP108-p |
| 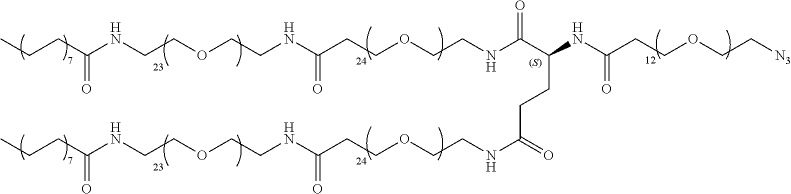 | LP109-p |
| 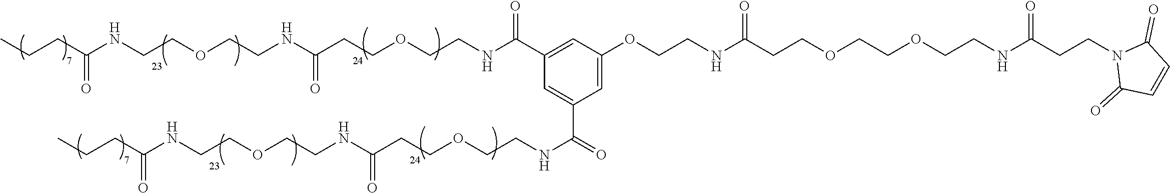 | LP110-p |
| 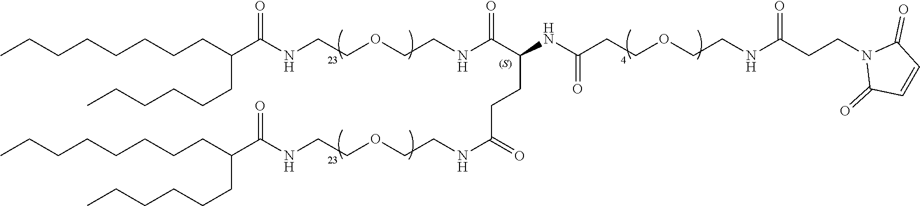 | LP111-p |
| 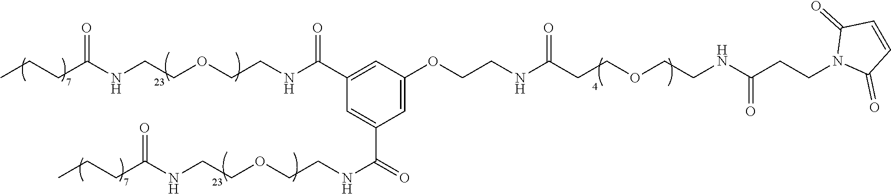 | LP124-p |
| 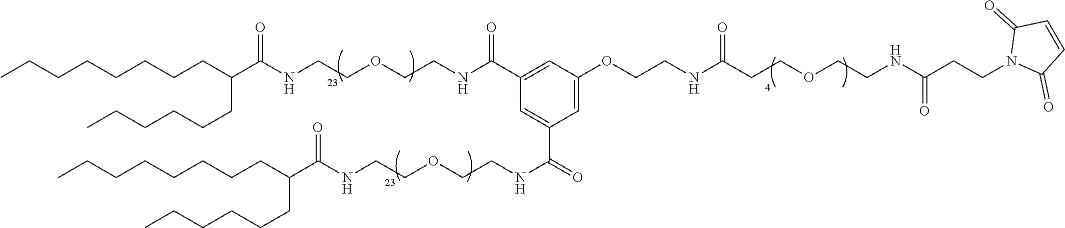 | LP130-p |
| 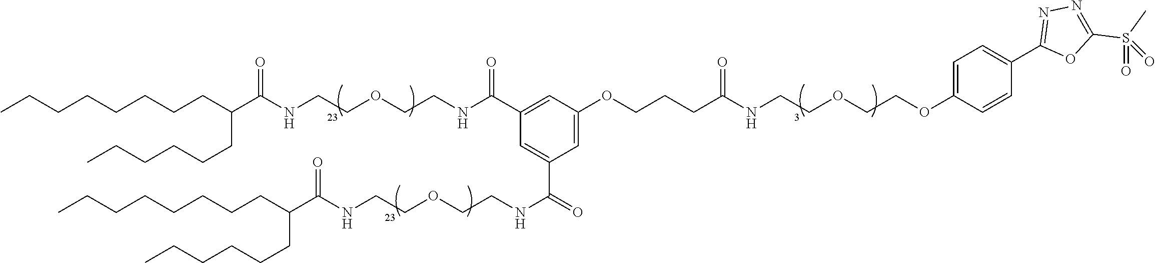 | LP143-p |
| 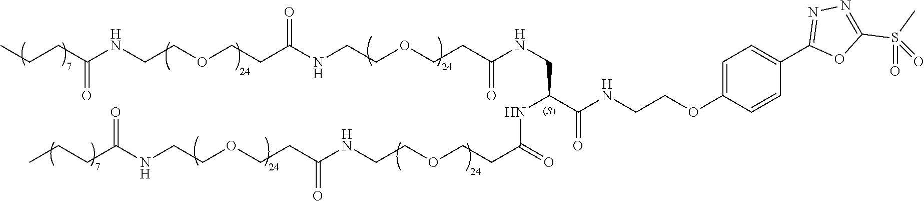 | LP210-p |
| 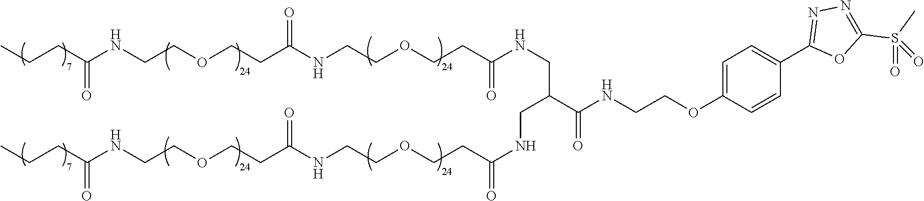 | LP217-p |
| 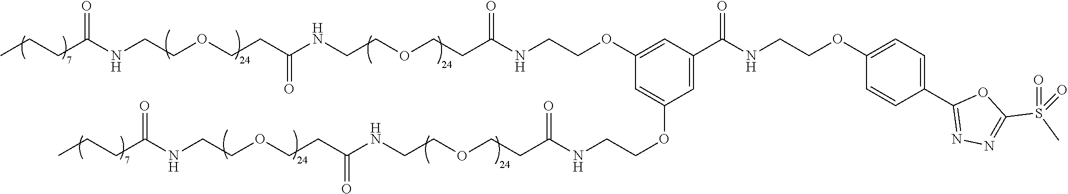 | LP220-p |
| 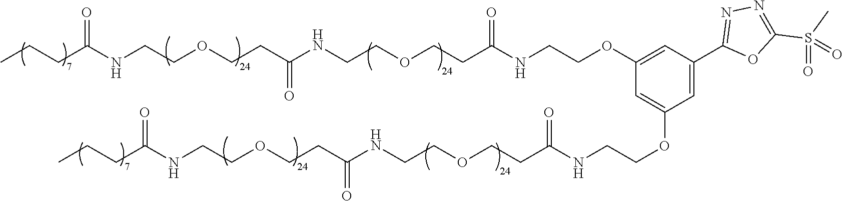 | LP221-p |
| 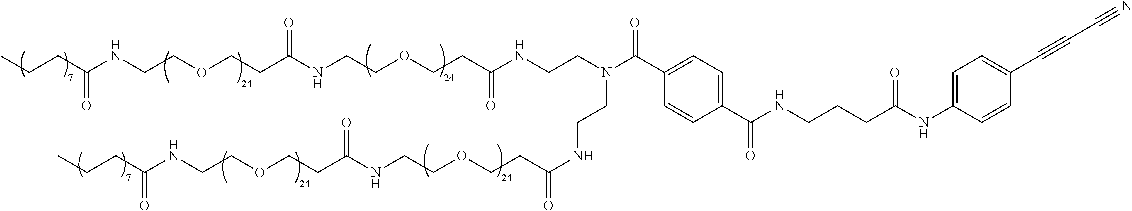 | LP223-p |
| 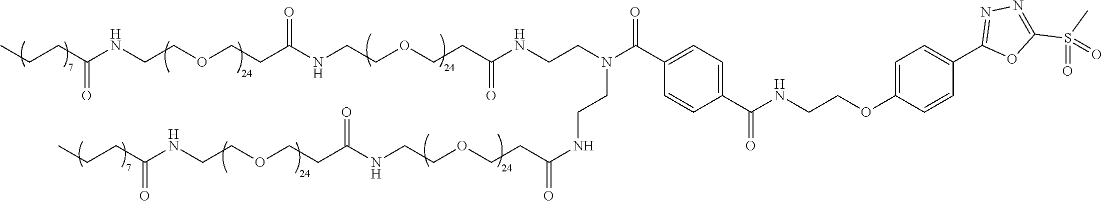 | LP224-p |
| 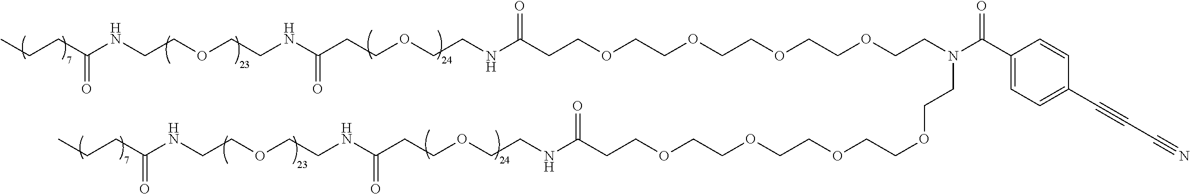 | LP225-p |
| 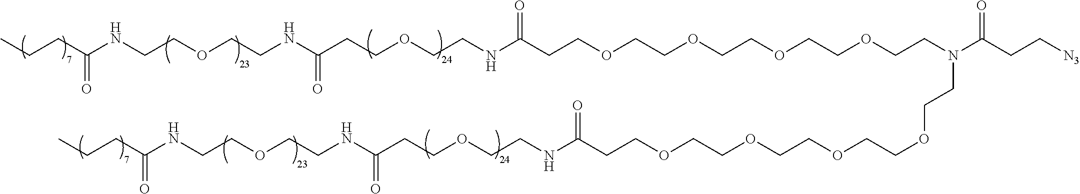 | LP226-p |
| 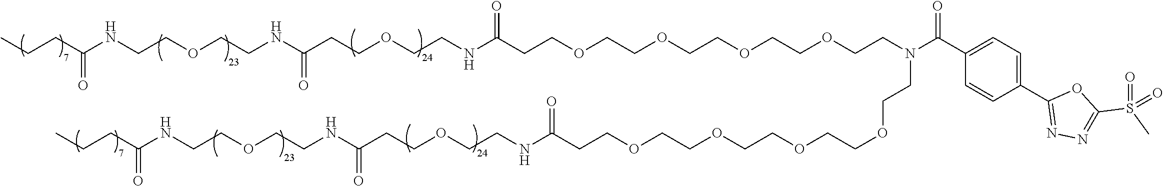 | LP238-p |
| 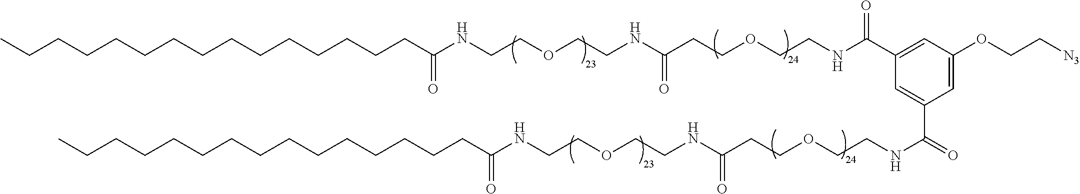 | LP240-p |
| 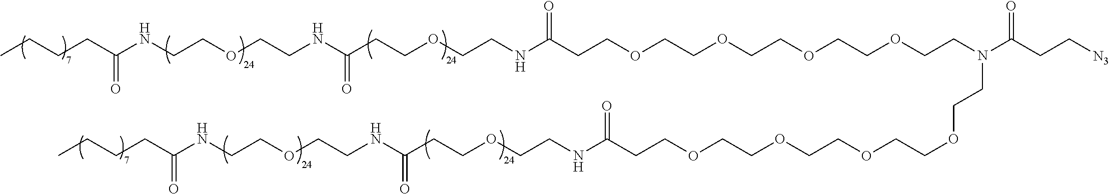 | LP246-p |
| 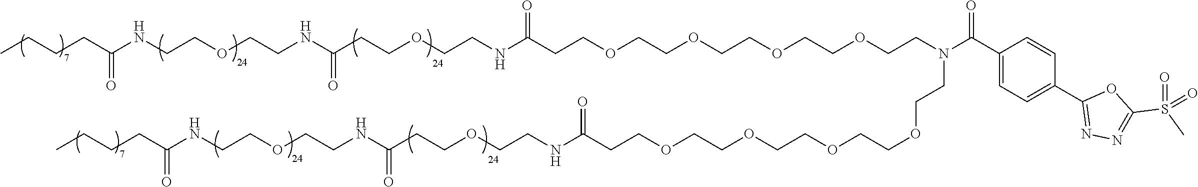 | LP247-p |
| 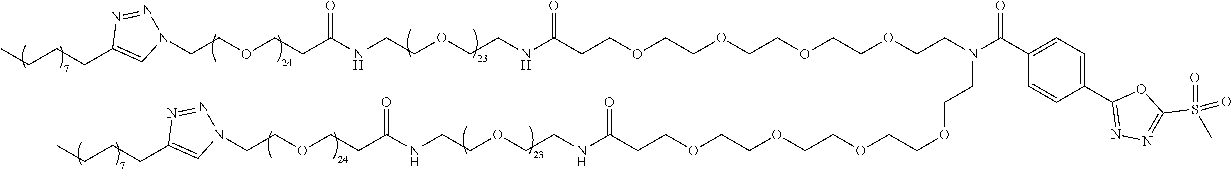 | LP339-p |
| 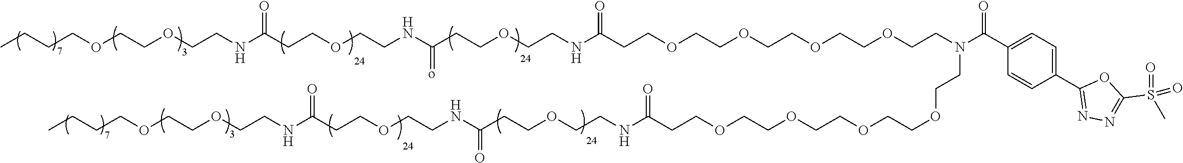 | LP340-p |
| 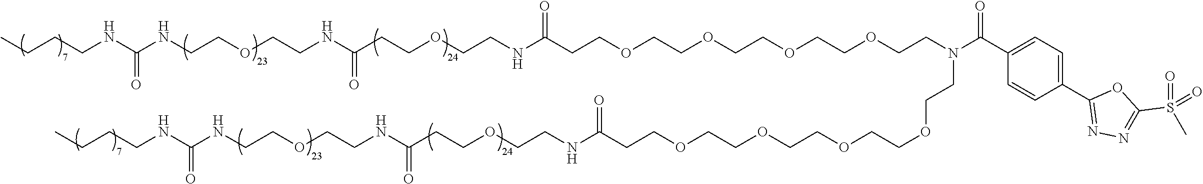 | LP357-p |
| 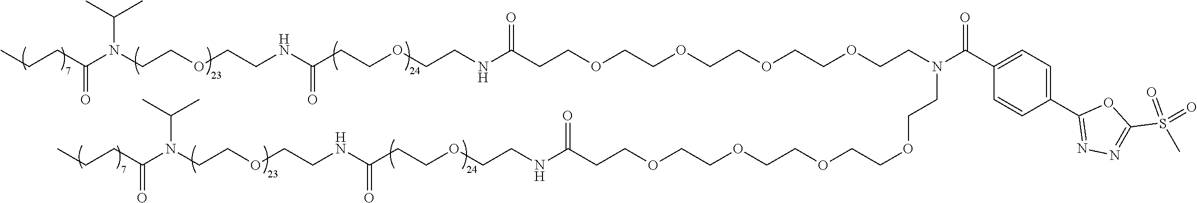 | LP358-p |

or a pharmaceutically acceptable salt of any of these lipid PK/PD modulator precursors.

In another aspect of the invention, the lipid PK/PD modulator precursor may be selected from the group consisting of the lipid PK/PD modulator precursors identified in Table 26.

TABLE 26
Example lipid PK/PD modulator precursors of the present invention
(compound name appears before structure.)

| | |
|---|---|
| 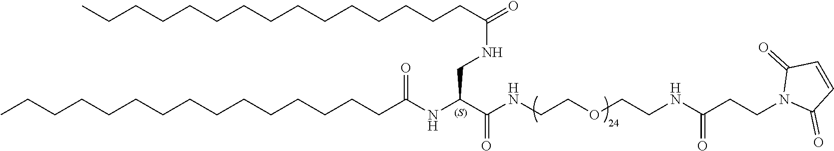 | LP5-p |
| 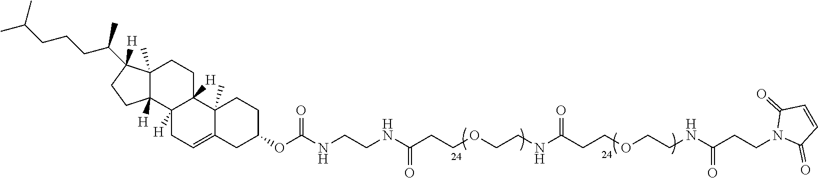 | LP33-p |
| 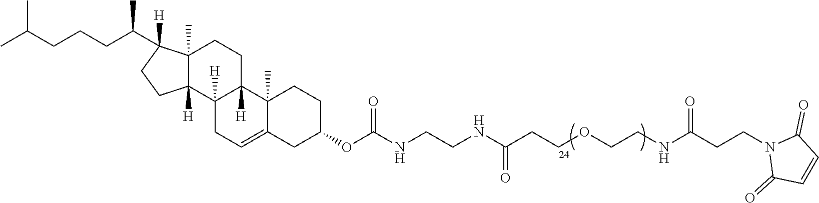 | LP81-p |
| 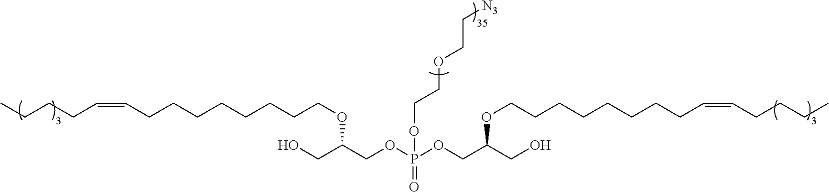 | LP105-p |

or a pharmaceutically acceptable salt of any of these lipid PK/PD modulator precursors.

In some embodiments, delivery vehicles may comprise one or more PK/PD modulators. In some embodiments, delivery vehicles comprise one, two, three, four, five, six, seven or more PK/PD modulators.

PK/PD modulator precursors may be conjugated to an RNAi agent using any known method in the art. In some embodiments, PK/PD modulator precursors comprising a maleimide moiety may be reacted with RNAi agents comprising a disulfide linkage to form a compound comprising a PK/PD modulator conjugated to an RNAi agent. The disulfide may be reduced, and added to a maleimide by way of a Michael-Addition reaction. An example reaction scheme is shown below:

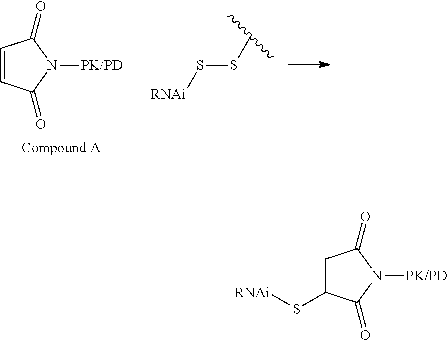

wherein Compound A is a PK/PD modulator precursor that comprises a maleimide moiety, RNAi comprises an RNAi agent, and [symbol] indicates a point of connection to any suitable group known in the art. In some embodiments of the reaction scheme above,

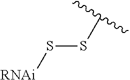

is attached to an alkyl group such as hexyl (C₆H₁₃).

In some embodiments, PK/PD modulator precursors may comprise a sulfone moiety and may react with a disulfide. An example reaction scheme is shown below:

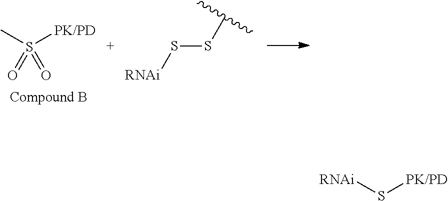

wherein Compound B is a PK/PD modulator precursor that comprises a sulfone moiety, RNAi comprises an RNAi agent, and [symbol] indicates a point of connection to any suitable group known in the art. In some instances of the reaction scheme above,

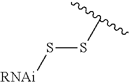

is attached to an alkyl group such as hexyl (C₆H₁₃).

In some embodiments, PK/PD modulator precursors may comprise an azide moiety and be reacted with an RNAi agent comprising an alkyne to form a compound comprising a PK/PD modulator conjugated to an RNAi agent according to the general reaction scheme below:

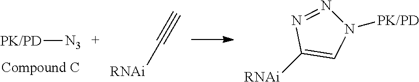

wherein Compound C is a PK/PD modulator precursor that comprises an azide moiety, and RNAi comprises an RNAi agent.

In some embodiments, PK/PD modulator precursors may comprise an alkyne moiety and be reacted with an RNAi agent comprising a disulfide to form a compound comprising a PK/PD modulator conjugated to an RNAi agent according to the general reaction scheme below:

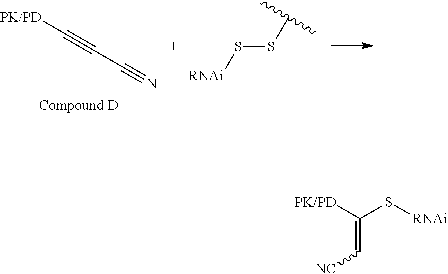

wherein Compound D is a PK/PD modulator precursor that comprises an alkyne, RNAi comprises an RNAi agent, and [symbol] indicates a point of connection to any suitable group known in the art. In some instances of the reaction scheme above,

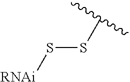

is attached to an alkyl group such as hexyl (C₆H₁₃).

In some embodiments, PK/PD modulators may be conjugated to the 5′ end of the sense or antisense strand, the 3′ end of the sense or antisense strand, or to an internal nucleotide of an RNAi agent. In some embodiments, an RNAi agent is synthesized with a disulfide-containing moiety at the 3′ end of the sense strand, and a PK/PD modulator precursor may be conjugated to the 3′ end of the sense strand using any of the appropriate general synthetic schemes shown above.

Examples of PK/PD modulators that may be covalently linked to an RNAi agent are shown in Table 27 below.

TABLE 27
Examples of PK/PD modulators that may be covalently linked to an RNAi agent.

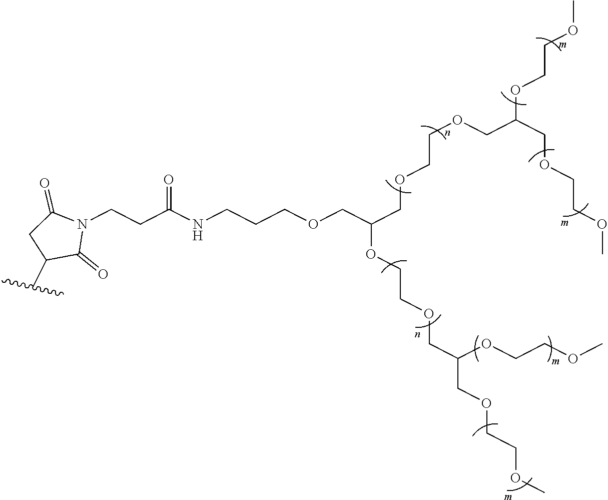

PEG40K (2 × 2-arm),
wherein n and m are each independently integers, and the molecular weight of the sum of all
PEG units is about kilodaltons

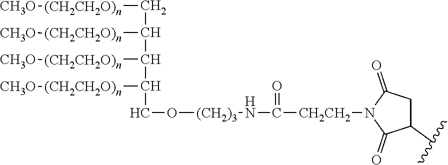

PEG40K (4-arm),
wherein n is an integer, and the molecular weight of the sum of all PEG units is about 40
kilodaltons

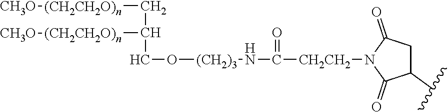

PEG40K (2-arm),
wherein n is an integer, and the molecular weight of the sum of all PEG units is about 40
kilodaltons

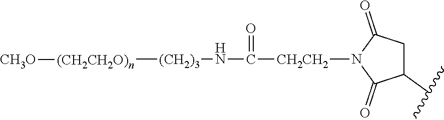

PEG40K,
wherein n is an integer, and the molecular weight of the sum of all PEG units is about 40
kilodaltons

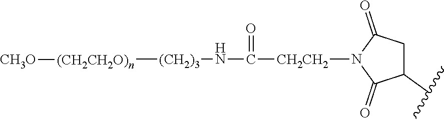

PEG10K,
wherein n is an integer, and the molecular weight of the sum of all PEG units is about 10
kilodaltons

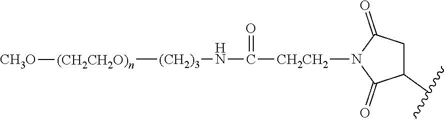

PEG5K,
wherein n is an integer, and the molecular weight of the sum of all PEG units is about 5
kilodaltons

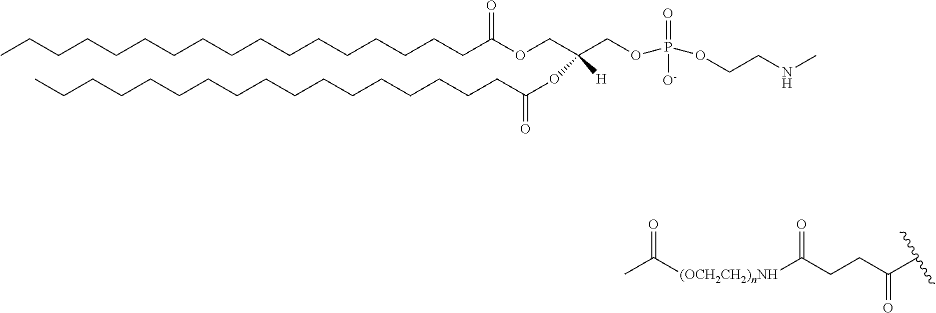

DSPE-PEG5K-NHS
wherein n is an integer, and the molecular weight of the sum of all PEG units is about 5
kilodaltons

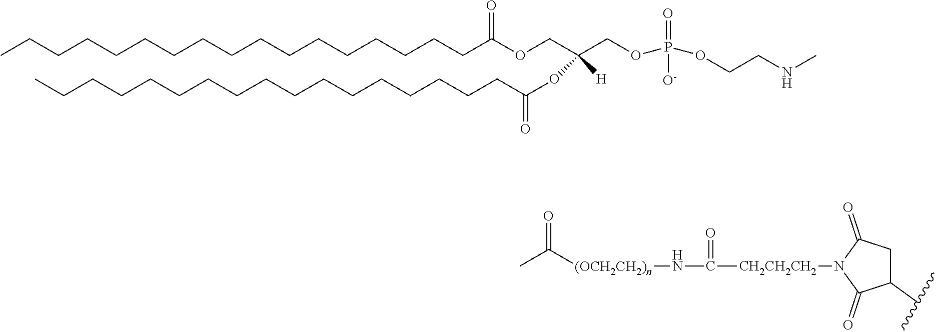

DSPE-PEG5K-MAL
Wherein n is an integer, and the molecular weight of the sum of all PEG units is about 5
kilodaltons

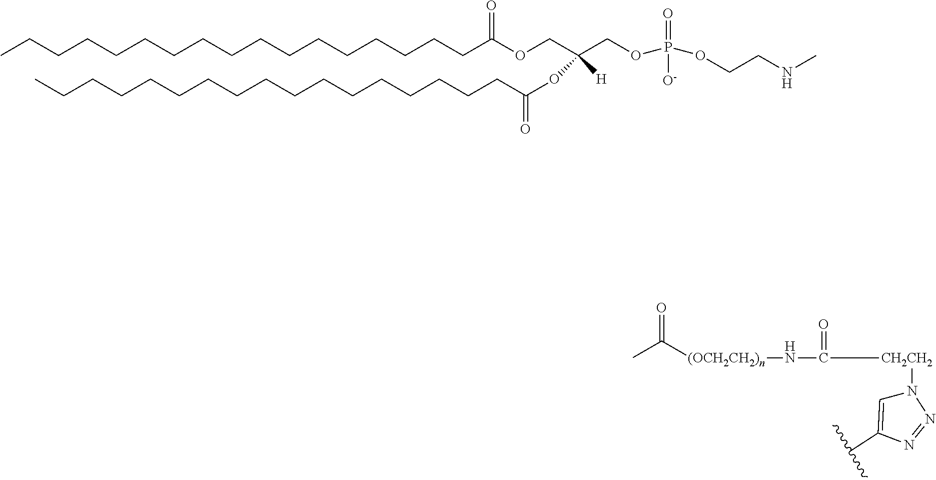

DSPE-PEG5K-N3
wherein n is an integer, and the molecular weight of the sum of all PEG units is about 5
kilodaltons

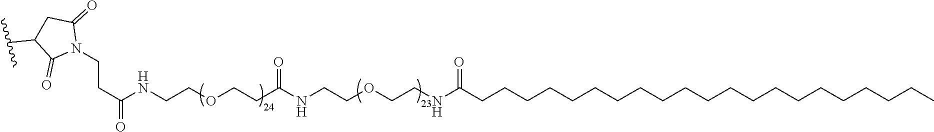

PEG47 + C22

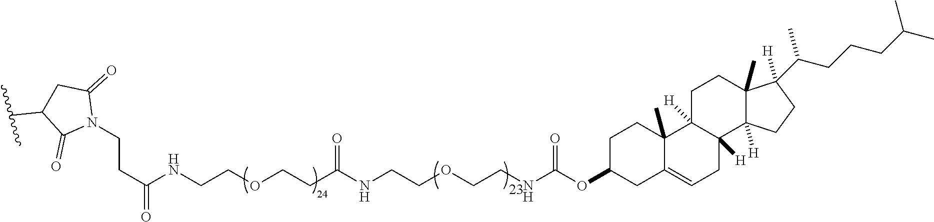

PEG47 + CLS (cholesterol)

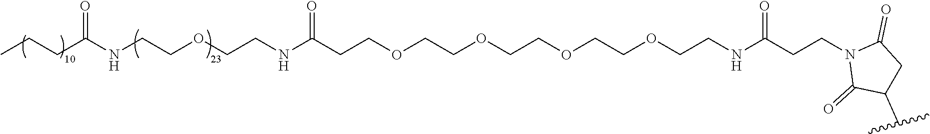

PEG23 + C22

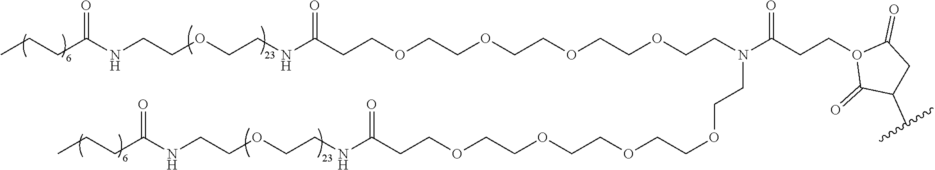

Bis(PEG23 + C14)

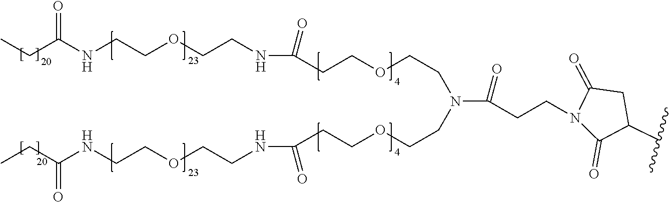

Bis(PEG23 + C22)

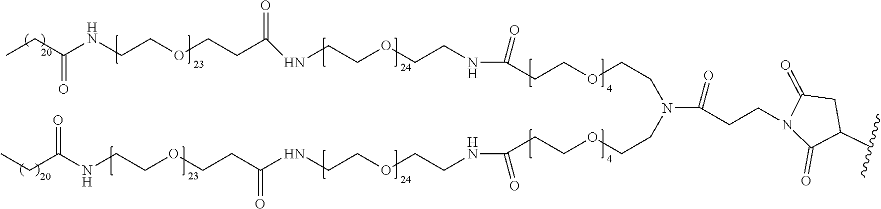

Bis(PEG47 + C22)

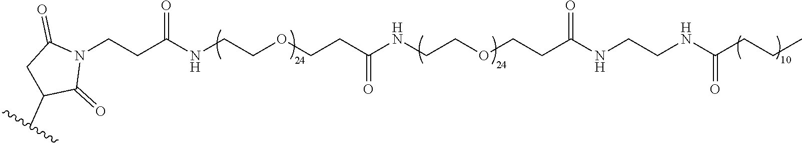

PEG48 + C22

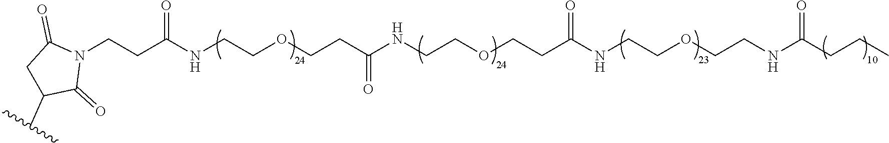

PEG71 + C22

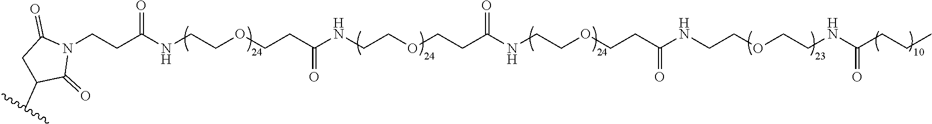

PEG95 + C22

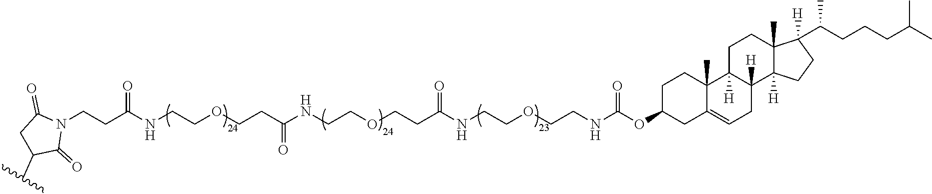

PEG71 + CLS

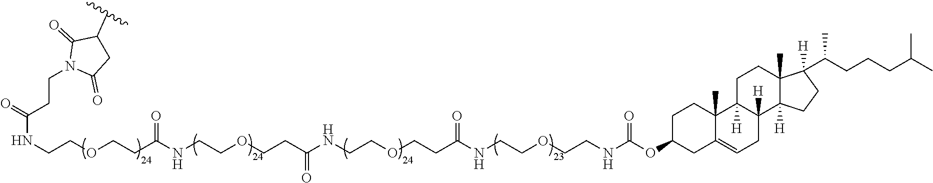

PEG95 + CLS

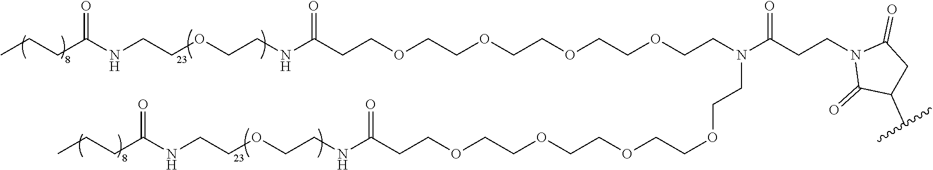

Bis(PEG23 + C18)

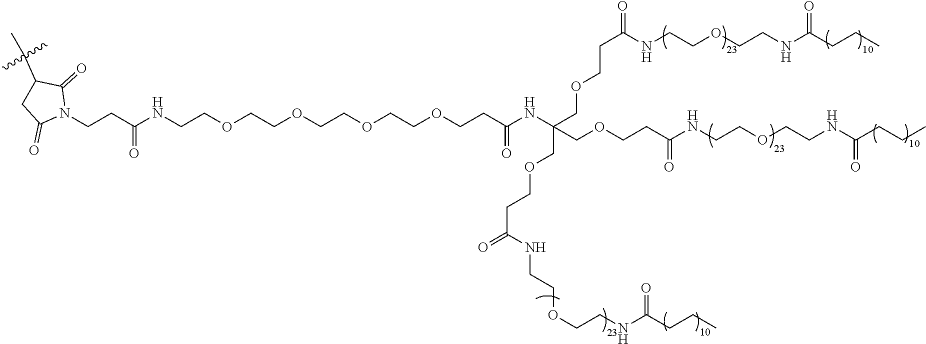

Tris(PEG23 + C22)

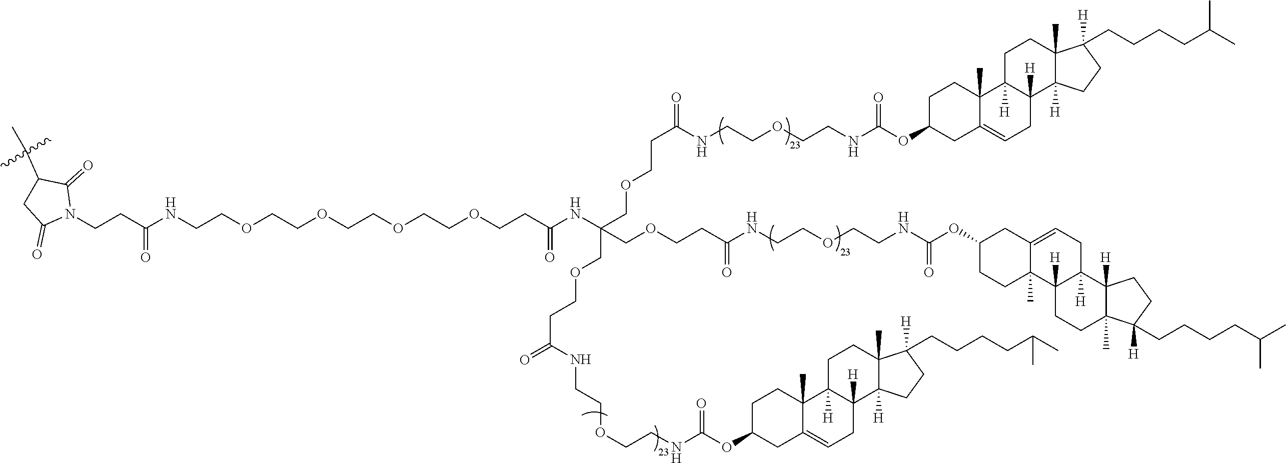

Tris(PEG23 + CLS)

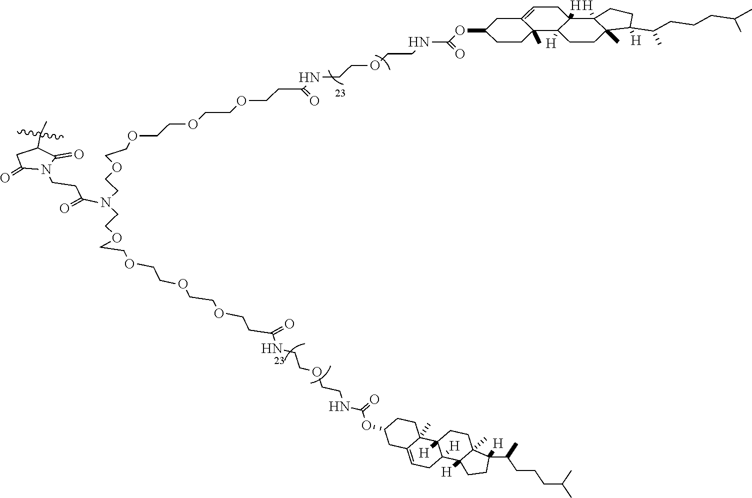

Bis(PEG23 + CLS)

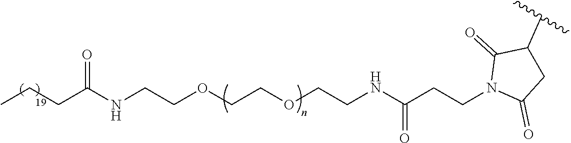

PEG5K + C22
wherein n is an integer, and the molecular weight of the sum of all PEG units is about 5
kilodaltons

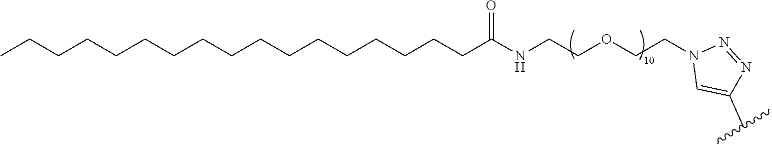

C18

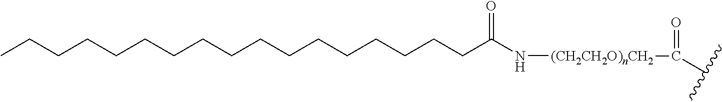

(NHS)-PEG1K + C18
wherein n is an integer, and the molecular weight of the sum of all PEG units is about 1
kilodalton

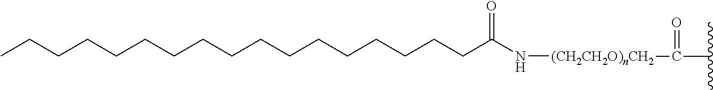

(NHS)-PEG2K + C18
wherein n is an integer, and the molecular weight of the sum of all PEG units is about 2
kilodaltons

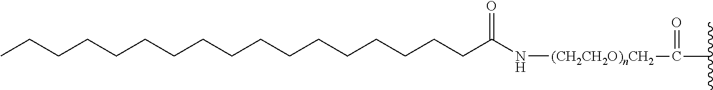

(NHS)-PEG5K + C18
wherein n is an integer, and the molecular weight of the sum of all PEG units is about 5
kilodaltons

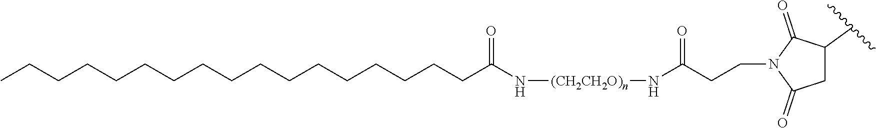

(MAL)-PEG5K + C18
wherein n is an integer, and the molecular weight of the sum of all PEG units is about 5
kilodaltons

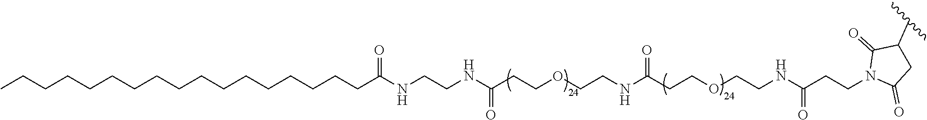

PEG48 + C18

or a pharmaceutically acceptable salt of any of these PK/PD modulators, wherein [symbol] indicates a point of connection to the RNAi agent.

### Linking Groups and Delivery Vehicles

In some embodiments, an RNAi agent contains or is conjugated to one or more non-nucleotide groups including, but not limited to a linking group a delivery polymer, or a delivery vehicle. The non-nucleotide group can enhance targeting, delivery, or attachment of the RNAi agent. Examples of linking groups are provided in Table 28. The non-nucleotide group can be covalently linked to the 3′ and/or 5′ end of either the sense strand and/or the antisense strand. In some embodiments, an RNAi agent contains a non-nucleotide group linked to the 3′ and/or 5′ end of the sense strand. In some embodiments, a non-nucleotide group is linked to the 5′ end of an RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

The RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the RNAi agents disclosed herein are synthesized having an NH₂-C₆ group at the 5′-terminus of the sense strand of the RNAi agent. The terminal amino group subsequently can be reacted to form a conjugate with, for example, a group that includes a compound having affinity for one or more integrins (i.e., and integrin targeting ligand) or a PK/PD modulator. In some embodiments, the RNAi agents disclosed herein are synthesized having one or more alkyne groups at the 5′-terminus of the sense strand of the RNAi agent. The terminal alkyne group(s) can subsequently be reacted to form a conjugate with, for example, a group that includes a targeting ligand.

In some embodiments, a targeting group comprises an integrin targeting ligand. In some embodiments, an integrin targeting ligand includes a compound that has affinity to integrin alpha-v-beta 6 The use of an integrin targeting ligands can facilitate cell-specific targeting to cells having the respective integrin on its respective surface, and binding of the integrin targeting ligand can facilitate entry of the RNAi agent, to which it is linked, into cells such as skeletal muscle cells. Targeting ligands, targeting groups, and/or PK/PD modulators can be attached to the 3′ and/or 5′ end of the RNAi agent, and/or to internal nucleotides on the RNAi agent, using methods generally known in the art. The preparation of targeting ligand and targeting groups, such as integrin αvβ6 is described in Example 3 below.

Embodiments of the present disclosure include pharmaceutical compositions for delivering an RNAi agent to a skeletal muscle cell in vivo. Such pharmaceutical compositions can include, for example, an RNAi agent conjugated to a targeting group that comprises an integrin targeting ligand that has affinity for integrin αvβ6. In some embodiments, the targeting ligand is comprised of a compound having affinity for integrin αvβ6.

In some embodiments, the RNAi agents disclosed herein can reduce gene expression in one or more of the following tissues: triceps, biceps, quadriceps, gastrocnemius, soleus, EDL (extensor digitorum longus), TA (Tibialis anterior), and/or diaphragm.

In some embodiments, the RNAi agent is synthesized having present a linking group, which can then facilitate covalent linkage of the RNAi agent to a targeting ligand, a targeting group, a PK/PD modulator, or another type of delivery polymer or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: Alk-SMPT-C6, Alk-S S—C6, DBCO-TEG, Me-Alk-SS—C6, and C6-S S-Alk-Me, reactive groups such a primary amines and alkynes, alkyl groups, abasic residues/nucleotides, amino acids, trialkyne functionalized groups, ribitol, and/or PEG groups.

A linker or linking group is a connection between two atoms that links one chemical group (such as an RNAi agent) or segment of interest to another chemical group (such as a targeting ligand, targeting group, PK/PD modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to RNAi agents without the use of an additional linker. In some embodiments, the targeting group is designed having a linker readily present to facilitate the linkage to an RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group may be conjugated synthetically to the 5′ or 3′ end of the sense strand of an RNAi agent described herein. In some embodiments, a linking group is conjugated synthetically to the 5′ end of the sense strand of an RNAi agent. In some embodiments, a linking group conjugated to an RNAi agent may be a trialkyne linking group.

Examples of certain modified nucleotides and linking groups, are provided in Table 28.

TABLE 28
Structures Representing Various Modified Nucleotides and Linking Groups

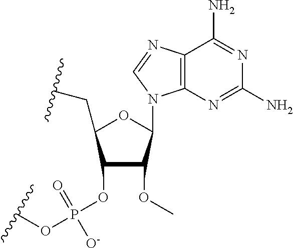

a_2N

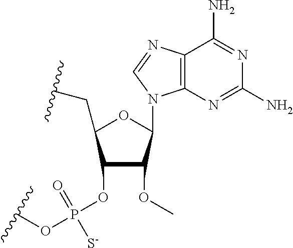

2_2Ns

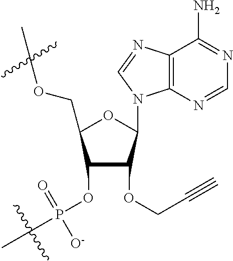

aAlk

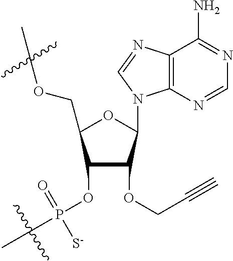

aAlks

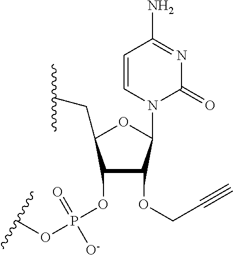

cAlk

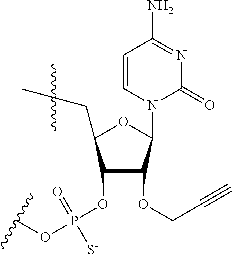

cAlks

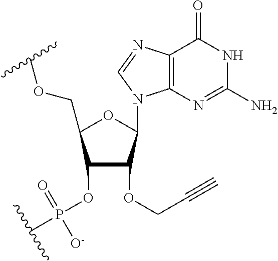

gAlk

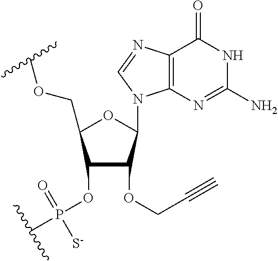

gAlks

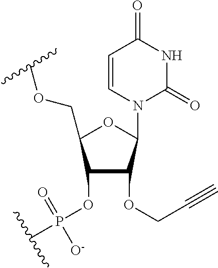

uAlk

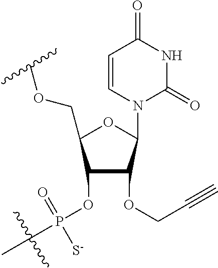

aAlks

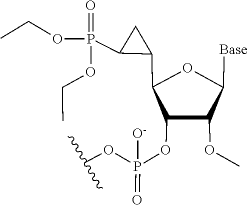

cPrp
When positioned internally in oligonucleotide:

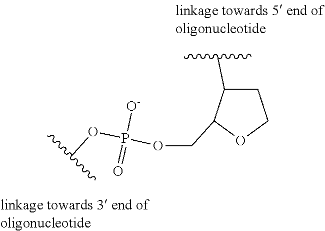 [SMILES: *OP(=O)([O-])OCC1OCCC1*]

(invAb)
When positioned internally in oligonucleotide:

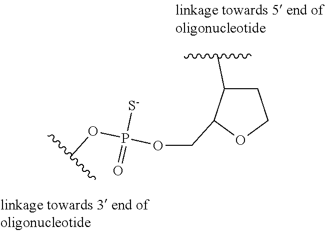 [SMILES: *OP(=O)([S-])OCC1OCCC1*]

(invAb)s
When positioned at the 3′ terminal end of oligonucleotide:

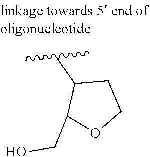 [SMILES: *C1CCOC1CO]

(invAb)
When positioned at the 3′ terminal end of oligonucleotide:

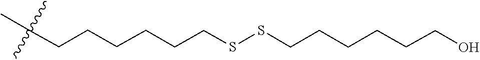 [SMILES: *CCCCCCSSCCCCCCO]

(C6-SS-C6)
When positioned internally in oligonucleotide:

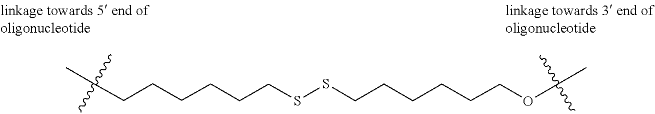 [SMILES: *CCCCCCSSCCCCCCO*]

(C6-SS-C6)
When positioned at the 3′ terminal end of oligonucleotide:

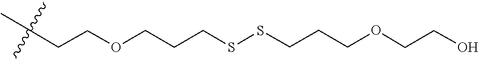 [SMILES: *CCOCCCSSCCCOCCO]

(6-SS-6)
When positioned internally in oligonucleotide:

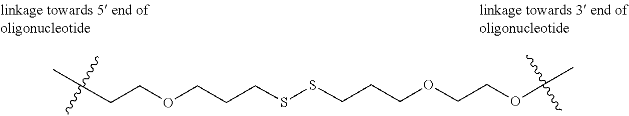 [SMILES: *CCOCCCSSCCCOCCO*]

(6-SS-6)

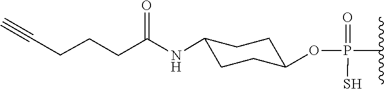 [SMILES: *P(=O)(S)OC1CCC(NC(=O)CCCC#C)CC1]

(Alk-cyHex)s

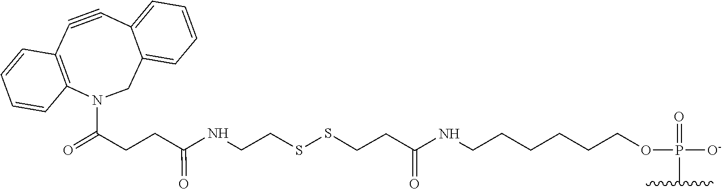 [SMILES: *P(=O)([O-])OCCCCCCNC(=O)CCSSCCNC(=O)CCC(=O)N1Cc2ccccc2C#Cc2ccccc21]

(C6-SS-Alk) or (Alk-SS-C6)

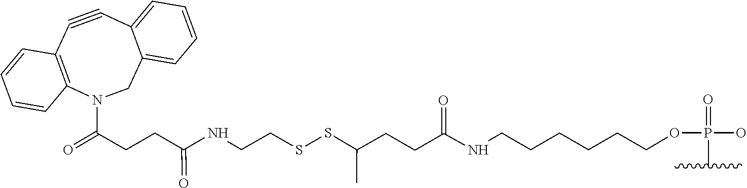

(C6-SS-Alk-Me)

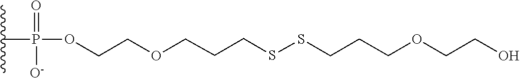 [SMILES: *P(=O)([O-])OCCOCCCSSCCCOCCO]

(PEG-C3-SS)

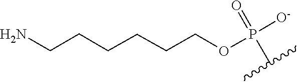 [SMILES: *P(=O)([O-])OCCCCCCN]

(NH2-C6)

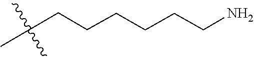 [SMILES: *CCCCCCN]

(C6-NH2)

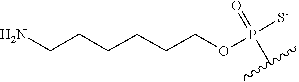 [SMILES: *P(=O)([S-])OCCCCCCN]

(NH2-C6)s

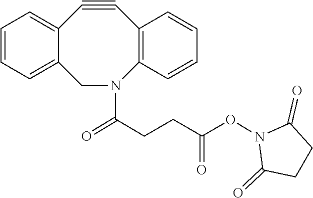 [SMILES: O=C(CCC(=O)N1Cc2ccccc2C#Cc2ccccc21)ON1C(=O)CCC1=O]

DBCO-NHS (BroadPharm ® BP-22231)

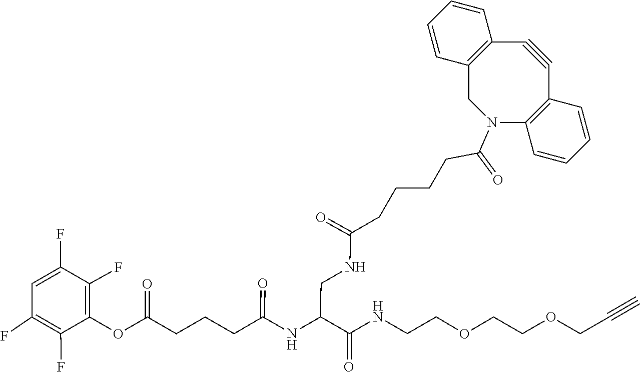 [SMILES: C#CCOCCOCCNC(=O)C(CNC(=O)CCCCC(=O)N1Cc2ccccc2C#Cc2ccccc21)NC(=O)CCCC(=O)Oc1c(F)c(F)cc(F)c1F]

Linker-1

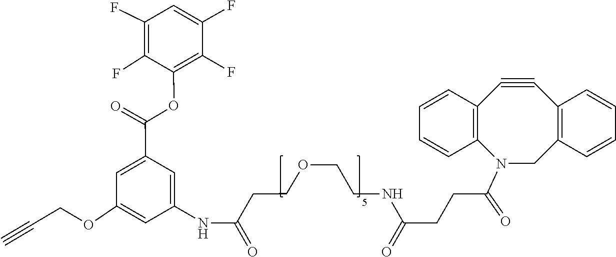

Linker-2

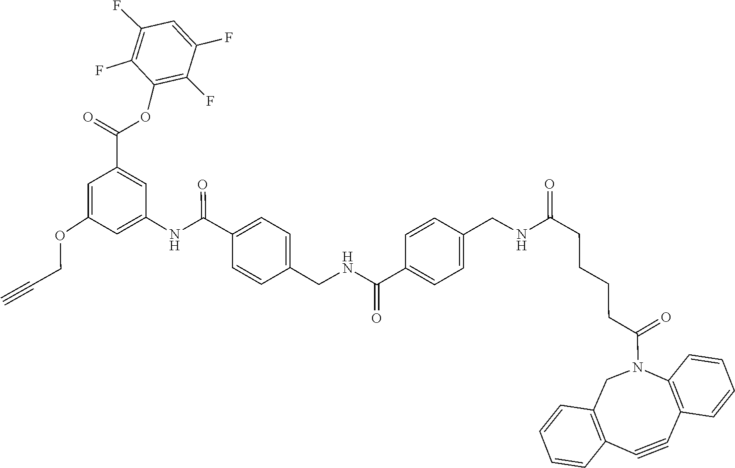 [SMILES: C#CCOc1cc(NC(=O)c2ccc(CNC(=O)c3ccc(CNC(=O)CCCCC(=O)N4Cc5ccccc5C#Cc5ccccc54)cc3)cc2)cc(C(=O)Oc2c(F)c(F)cc(F)c2F)c1]

Linker-3

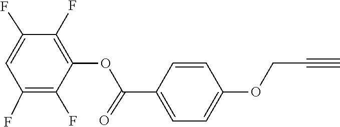

Linker-4

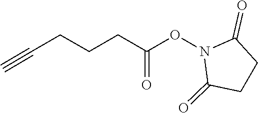

Linker-5 (Active Scientific ® AS28942)

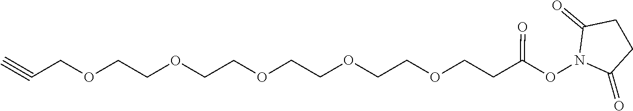 [SMILES: C#CCOCCOCCOCCOCCOCCC(=O)ON1C(=O)CCC1=O]

Linker-6 (BroadPharm ® BP-20907)

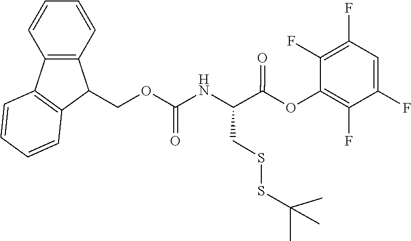 [SMILES: CC(C)(C)SSC[C@H](NC(=O)OCC1c2ccccc2-c2ccccc21)C(=O)Oc1c(F)c(F)cc(F)c1F]

Linker-7

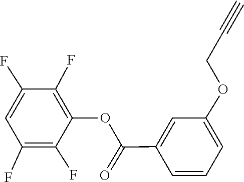 [SMILES: C#CCOc1cccc(C(=O)Oc2c(F)c(F)cc(F)c2F)c1]

Linker-8

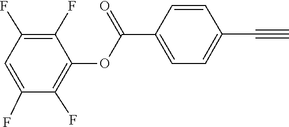 [SMILES: C#Cc1ccc(C(=O)Oc2c(F)c(F)cc(F)c2F)cc1]

Linker-9

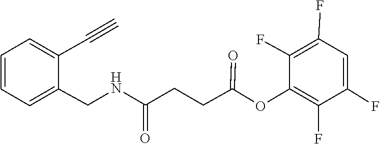 [SMILES: C#Cc1ccccc1CNC(=O)CCC(=O)Oc1c(F)c(F)cc(F)c1F]

Linker-10

Alternatively, other linking groups known in the art may be used.

In addition or alternatively to linking an RNAi agent to one or more targeting ligands, targeting groups, and/or PK/PD modulators, in some embodiments, a delivery vehicle may be used to deliver an RNAi agent to a cell or tissue. A delivery vehicle is a compound that can improve delivery of the RNAi agent to a cell or tissue, and can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/022309, WO 2011/104169, and WO 2012/083185, WO 2013/032829, WO 2013/158141, each of which is incorporated herein by reference), or other delivery systems available in the art.

### Pharmaceutical Compositions

In some embodiments, the present disclosure provides pharmaceutical compositions that include, consist of, or consist essentially of, one or more of the delivery platforms disclosed herein.

As used herein, a “pharmaceutical composition” comprises a pharmacologically effective amount of an Active Pharmaceutical Ingredient (API), and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions described herein can contain other additional components commonly found in pharmaceutical compositions. In some embodiments, the additional component is a pharmaceutically-active material. Pharmaceutically-active materials include, but are not limited to: anti-pruritics, astringents, local anesthetics, or anti-inflammatory agents (e.g., antihistamine, diphenhydramine, etc.), small molecule drug, antibody, antibody fragment, aptamers, and/or vaccines.

The pharmaceutical compositions may also contain preserving agents, solubilizing agents, stabilizing agents, wetting agents, emulsifiers, sweeteners, colorants, odorants, salts for the variation of osmotic pressure, buffers, coating agents, or antioxidants. They may also contain other agent with a known therapeutic benefit.

The pharmaceutical compositions can be administered in a number of ways depending upon whether local or systemic treatment is desired and upon the area to be treated. Administration can be made by any way commonly known in the art, such as, but not limited to, topical (e.g., by a transdermal patch), pulmonary (e.g., by inhalation or insufflation of powders or aerosols, including by nebulizer, intratracheal, intranasal), epidermal, transdermal, oral or parenteral. Parenteral administration includes, but is not limited to, intravenous, intraarterial, subcutaneous, intraperitoneal or intramuscular injection or infusion; subdermal (e.g., via an implanted device), intracranial, intraparenchymal, intrathecal, and intraventricular, administration. In some embodiments, the pharmaceutical compositions described herein are administered by subcutaneous injection. The pharmaceutical compositions may be administered orally, for example in the form of tablets, coated tablets, dragées, hard or soft gelatin capsules, solutions, emulsions or suspensions. Administration can also be carried out rectally, for example using suppositories; locally or percutaneously, for example using ointments, creams, gels, or solutions; or parenterally, for example using injectable solutions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of any of the ligands described herein that can be in microcrystalline form, for example, in the form of an aqueous microcrystalline suspension. Liposomal formulations or biodegradable polymer systems can also be used to present any of the ligands described herein for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). As used herein, “pharmacologically effective amount,” “therapeutically effective amount,” or simply “effective amount” refers to that amount of an the pharmaceutically active agent to produce a pharmacological, therapeutic or preventive result.

Medicaments containing a RNAi agent are also an object of the present invention, as are processes for the manufacture of such medicaments, which processes comprise bringing one or more compounds containing a RNAi agent, and, if desired, one or more other substances with a known therapeutic benefit, into a pharmaceutically acceptable form.

The described RNAi agents and pharmaceutical compositions comprising RNAi agents disclosed herein may be packaged or included in a kit, container, pack, or dispenser. The RNAi agents and pharmaceutical compositions comprising the RNAi agents may be packaged in pre-filled syringes or vials.

### Methods of Treatment and Inhibition of Expression

The delivery platforms disclosed herein can be used to treat a subject (e.g., a human or other mammal) having a disease or disorder that would benefit from administration of the RNAi agent. In some embodiments, the delivery platforms for an RNAi agent disclosed herein can be used to treat a subject (e.g., a human) that would benefit from reduction and/or inhibition in expression of mRNA and/or a target protein levels, for example, a subject that has been diagnosed with or is suffering from symptoms related to muscular dystrophy.

In some embodiments, the subject is administered a therapeutically effective amount of any one or more RNAi agents. Treatment of a subject can include therapeutic and/or prophylactic treatment. The subject is administered a therapeutically effective amount of any one or more RNAi agents described herein. The subject can be a human, patient, or human patient. The subject may be an adult, adolescent, child, or infant. Administration of a pharmaceutical composition described herein can be to a human being or animal.

The RNAi agents described herein can be used to treat at least one symptom in a subject having a disease or disorder relating to a target gene, or having a disease or disorder that is mediated at least in part by target gene expression. In some embodiments, the RNAi agents are used to treat or manage a clinical presentation of a subject with a disease or disorder that would benefit from or be mediated at least in party by a reduction in target mRNA. The subject is administered a therapeutically effective amount of one or more of the RNAi agents or RNAi agent-containing compositions described herein. In some embodiments, the methods disclosed herein comprise administering a composition comprising an RNAi agent described herein to a subject to be treated. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by target gene expression, in a patient in need thereof, wherein the methods include administering to the patient any of the RNAi agents described herein.

In some embodiments, the gene expression level and/or mRNA level of a target gene in a subject to whom an RNAi agent is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the RNAi agent or to a subject not receiving the RNAi agent. The gene expression level and/or mRNA level in the subject may be reduced in a cell, group of cells, and/or tissue of the subject.

In some embodiments, the protein level in a subject to whom an RNAi agent has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the RNAi agent or to a subject not receiving the RNAi agent. The protein level in the subject may be reduced in a cell, group of cells, tissue, blood, and/or other fluid of the subject.

A reduction in mRNA levels and protein levels can be assessed by any methods known in the art. As used herein, a reduction or decrease in mRNA level and/or protein level are collectively referred to herein as a reduction or decrease in the target gene or inhibiting or reducing the expression of the target gene. The Examples set forth herein illustrate known methods for assessing inhibition of gene expression.

In some embodiments, RNAi agents may be used in the preparation of a pharmaceutical composition for use in the treatment of a disease, disorder, or symptom that is mediated at least in part by target gene expression. In some embodiments, the disease, disorder, or symptom that is mediated at least in part by target gene expression is muscular dystrophy.

In some embodiments, methods of treating a subject are dependent on the body weight of the subject. In some embodiments, RNAi agents may be administered at a dose of about 0.05 mg/kg to about 40.0 mg/kg of body weight of the subject. In other embodiments RNAi agents may be administered at a dose of about 5 mg/kg to about 20 mg/kg of body weight of the subject.

In some embodiments, RNAi agents may be administered in a split dose, meaning that two doses are given to a subject in a short (for example, less than 24 hour) time period. In some embodiments, about half of the desired daily amount is administered in an initial administration, and the remaining about half of the desired daily amount is administered approximately four hours after the initial administration.

In some embodiments, RNAi agents may be administered once a week (i.e., weekly). In other embodiments, RNAi agents may be administered biweekly (once every other week).

In some embodiments, RNAi agents or compositions containing RNAi agents may be used for the treatment of a disease, disorder, or symptom that is mediated at least in part by target gene expression. In some embodiments, the disease, disorder or symptom that is mediated at least in part by target gene expression is muscular dystrophy.

### Cells, Tissues, and Non-Human Organisms

Cells, tissues, and non-human organisms that include at least one of the delivery platforms comprising an RNAi agent described herein is contemplated. The cell, tissue, or non human organism is made by delivering the RNAi agent to the cell, tissue, or non-human organism by any means available in the art. In some embodiments, the cell is a mammalian cell, including, but not limited to, a human cell.

The above provided embodiments and items are now illustrated with the following, non-limiting examples.

### EXAMPLES

The following examples are not limiting and are intended to illustrate certain embodiments disclosed herein.

### Example 1. Synthesis of RNAi Agents and Compositions Containing RNAi Agents

The following describes the general procedures for the syntheses of certain RNAi agents, and conjugates thereof, that are illustrated in the non-limiting Examples set forth herein.

Synthesis of RNAi Agents. RNAi agents can be synthesized using methods generally known in the art. For the synthesis of the RNAi agents illustrated in the Examples set forth herein, the sense and antisense strands of the RNAi agents were synthesized according to phosphoramidite technology on solid phase used in oligonucleotide synthesis. Depending on the scale, a MerMade96E® (Bioautomation), a MerMade12® (Bioautomation), or an Oligopilot 100 (GE Healthcare) was used. Syntheses were performed on a solid support made of controlled pore glass (CPG, 500 Å or 600 Å, obtained from Prime Synthesis, Aston, PA, USA) or polystyrene (obtained from Kinovate, Oceanside, CA, USA). All RNA and 2′-modified RNA phosphoramidites were purchased from Thermo Fisher Scientific (Milwaukee, WI, USA), ChemGenes (Wilmington, MA, USA), or Hongene Biotech (Morrisville, NC, USA). Specifically, the following 2′-O-methyl phosphoramidites that were used include the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites and 2′-O-propargyl phosphoramidites carried the same protecting groups as the 2′-O-methyl phosphoramidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes. The following UNA phosphoramidites that were used included the following: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-dii s o-propyl)]-phosphoramidite. In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile or a 200 mM solution of xanthane hydride (TCI America, Portland, OR, USA) in pyridine was employed.

TFA aminolink phosphoramidites were also commercially purchased (ThermoFisher) to introduce the (NH2-C6) reactive group linkers. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ 0-Me), and 60 sec (2′ F). Trialkyne-containing phosphoramidites were synthesized to introduce the respective (TriAlk #) linkers. When used in connection with the RNAi agents presented in certain Examples herein, trialkyne-containing phosphoramidites were dissolved in anhydrous dichloromethane or anhydrous acetonitrile (50 mM), while all other amidites were dissolved in anhydrous acetonitrile (50 mM), and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ 0-Me), and 60 sec (2′ F).

For some RNAi agents, a linker, such as a C6-SS—C6 or a 6-SS-6 group, was introduced at the 3′ terminal end of the sense strand. Pre-loaded resin was commercially acquired with the respective linker. Alternatively, for some sense strands, a dT resin was used and the respectively linker was then added via standard phosphoramidite synthesis.

Cleavage and deprotection of support bound oligomer. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

Purification. Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G25 fine with a running buffer of 100 mM ammonium bicarbonate, pH 6.7 and 20% Acetonitrile or filtered water.

Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor and the dilution factor to determine the duplex concentration. The conversion factor used was either 0.037 mg/(mL·cm) or was calculated from an experimentally determined extinction coefficient.

### Example 2. Synthesis of Linking Agents

### Synthesis of Linker 1

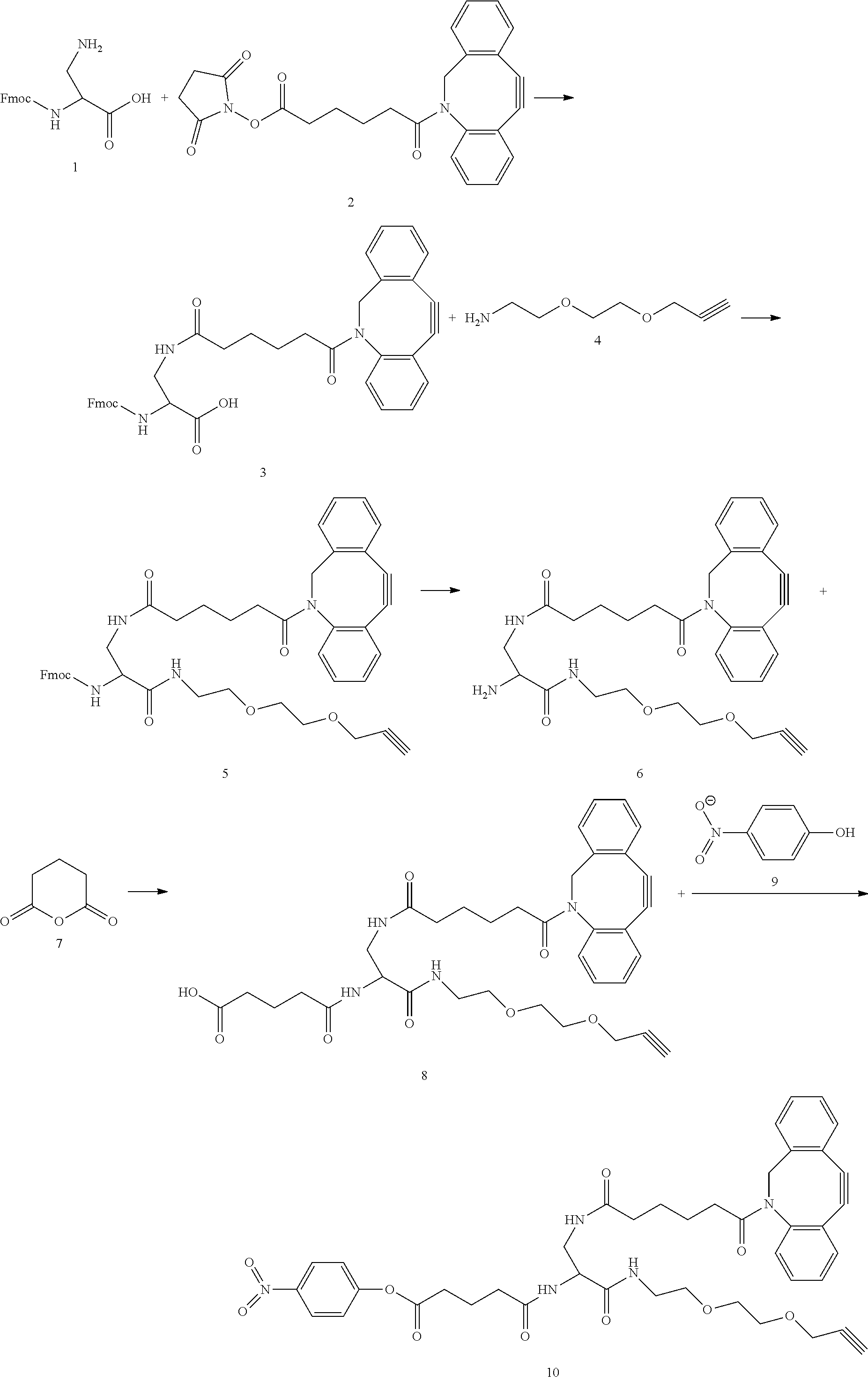

Compound 1 (423 mg) and compound 2 (516 mg) were mixed together in DMF, and DIPEA (0.26 ml) was added. The reaction was stirred overnight. The product was isolated through normal phase column chromatography to provide 450 mg of Compound 3.

Compound 3 (450 mg, lequiv) and Compound 4 (0.12 ml, 1.2equiv), TBTU (248 mg, 1.1equiv), and DIPEA (0.183 ml, 1.5 equiv) were mixed together in DMF. The reaction was stirred overnight. The product was isolated via normal phase column chromatography to provide compound 5.

Compound 5 was treated with 20% piperidine in DMF for half an hour. Product was isolated via normal phase column chromatography to provide compound 6.

Compound 6 (93 mg, lequiv) and 7 (25.9 mg, 1.3equiv), TEA (0.045 ml, 2 equiv) were mixed together in DCM. The reaction was stirred overnight. To this mixture compound 9 (57 mg) and EDC (72 mg) were added. The reaction was stirred overnight. The product was isolated through normal phase column chromatography to provide compound 10 (100 mg).

### Synthesis of Linker 2

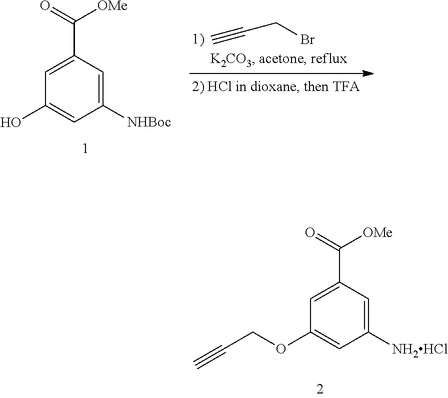

To a solution of 1 (1.69 g, 6.3 mmol) and propargyl bromide (1.499 g, 1.4 mL, d=1.57 g/mL, 12.6 mmol) in acetone (50 mL) was added K₂CO₃ (3.477 g, 25.2 mmol) at room temperature. The reaction mixture was stirred reflux for 3 hrs. Upon consumption of starting material, the reaction mixture was concentrated under vacuum, and dissolved with EA/hexane/DCM (30 mL each) and filtered. The mother liquor was concentrated, and the residue was purified by CombiFlash® using silica gel as the stationary phase and was eluted with a gradient of EtOAc in hexanes (0-50%). Yield of 2: 0.438 g (23%). [M-H] calculated for C₁₆H₁₈NO₅: 304.12, found: 304.46.

The compound (438 mg) above was dissolved into 4 M HCl in dioxane at room temperature for 5 hrs, and the reaction was monitored by LC-MS with only 50% conversion. The mixture was spun down and filtered. 2 mL of TFA was added into the solid, starting material was consumed after 2 hrs as monitored by LC-MS. The mixture was concentrated under vacuum. Yield, 333 mg, solid, 96%. [M+H] calculated for C₁₁H₁₂NO₃: 206.08, found: 206.26.

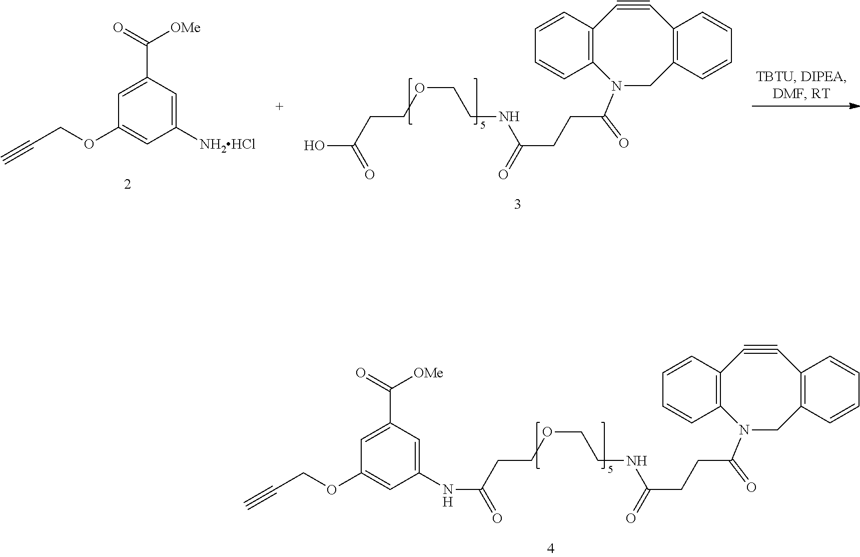

To a solution of TBTU (22.5 mg, 0.07 mmol), DBCO-PEG5-acid (50 mg, 0.084 mmol), N,N-diisopropylethylamine (27 mg, 36 μL, d=0.742 g/mL, 0.21 mmol) in DMF (0.8 mL) was added 2 (16.8 mg, 0.07 mmol). The reaction mixture was stirred at room temperature. After confirming all starting material was consumed by LC-MS, the reaction mixture was quenched by 2 mL of saturated NaHCO₃ aqueous solution and extracted with ethyl acetate (10 mL×3). The combined organic layer was washed with HCl (aq) and brine sequentially. The organic layer was dried over Na₂SO₄ and concentrated under high vacuum. The crude was loaded on to a silica column and purified (MPA: DCM, MPB: 10% MeOH in DCM, 0-30% ramp in 30 min) to afford the product. Yield: 76.5 mg, 99%. [M+H] calculated for C₄₃H₅₀N₃O₁₁: 784.34, found: 784.83.

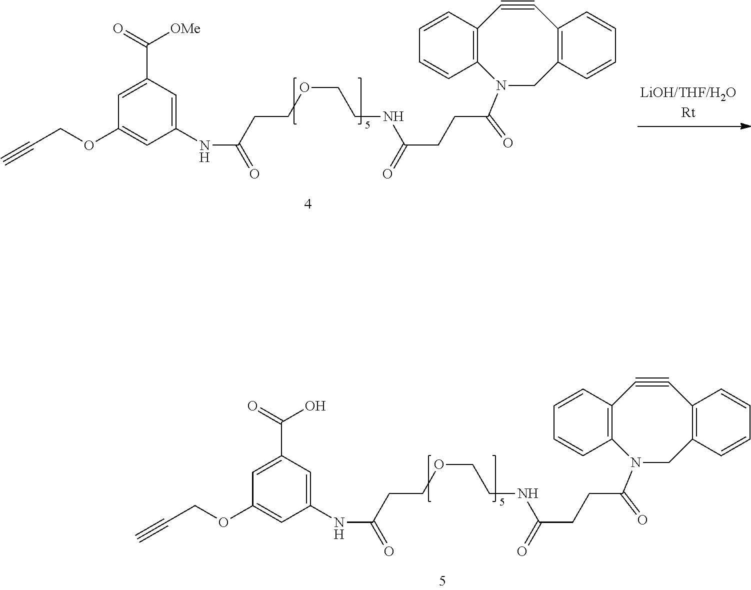

The product 4 was dissolved in 0.3 mL of THF/H₂O (2:1 v/v) and 55.6 mg of LiOH was added into the reaction. After stirring at room temperature overnight, the reaction mixture was filtered thought a short pad of silica gel. The filtrate was collected and concentrated under reduced pressure. The crude was loaded on to a silica column and purified (MPA: DCM, MPB: 10% MeOH in DCM, 0-50% ramp in 30 min) to afford the product. Yield: 42.9 mg. [M+H] calculated for C₄₂H₄₈N3011: 770.33, found: 770.91.

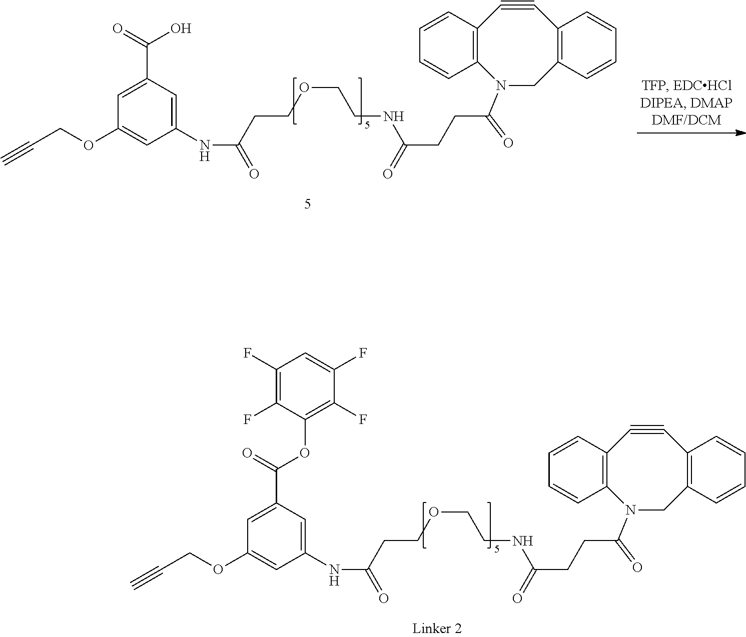

To a solution of 5 (43 mg, 0.056 mmol), 2,3,5,6-tetrafluorophenol (46.5 mg, 0.28 mmol), and N,N-diisopropylethylamine (144.5 mg, 0.19 mL, d=0.742 g/mL, 1.12 mmol) in DCM (2 mL) was added N-(3-Dimethylaminopropyl)-N′-ethylcarbodiimide hydrochloride (53.5 mg, 0.28 mmol). The reaction mixture was stirred at room temperature. After confirming by LC-MS that all starting material was consumed, the reaction mixture was concentrated by lyophilization, was loaded on to a silica column and purified (MPA: DCM, MPB: 20% McOH in DCM, 0-30% ramp in 30 min) to afford the product. Yield: 12 mg, 23%. [M+H] calculated for C₄₈H₄₈F₄N₃O₁₁: 918.32, found: 918.89.

### Synthesis of Linker 3

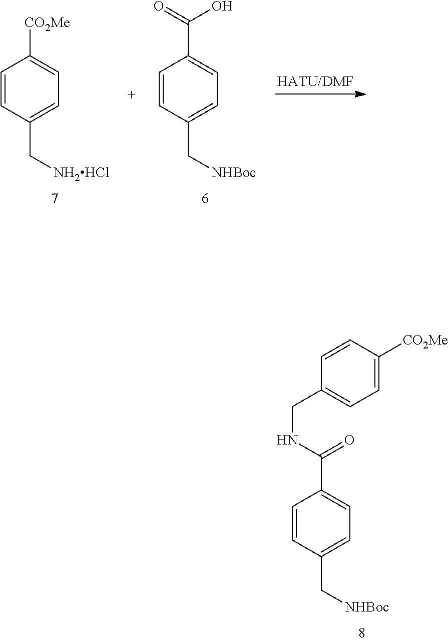

To a solution of acid 6 (1.2461 g, 4.9591 mmol), HATU (2.2613 g, 5.9509 mmol), and DIPEA (2.3030 g, 3.1 mL, d=0.742 g/mL, 17.8527 mmol, 3 eq) in DMF (4 mL) was added amine 7 (1 g, 4.9591 mmol)/DMF(1 mL), the reaction mixture was stirred at room temperature. After confirming by LC-MS that all starting material was consumed, the reaction mixture was concentrated by lyophilization, was loaded on to a silica column and purified (MPA: DCM, MPB: 10% MeOH in DCM, 0-30% ramp in 30 min) to afford the product 8. Yield: 1.3269 g, 67%. [M+H] calculated for C₂₂H₂₇N₂O₅: 399.19, found: 399.39.

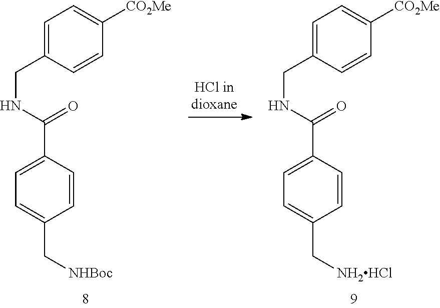

To a solution of 4 M HCl in dioxane was added compound 8 (1.3269 g). After stirring at room temperature for 1 h, the starting material was consumed completely. White cake was afforded by simple filtration. Yield, 630 mg, 63%. [M+H] calculated for C₁₇H₁₉N₂O₃: 299.14, found: 299.34.

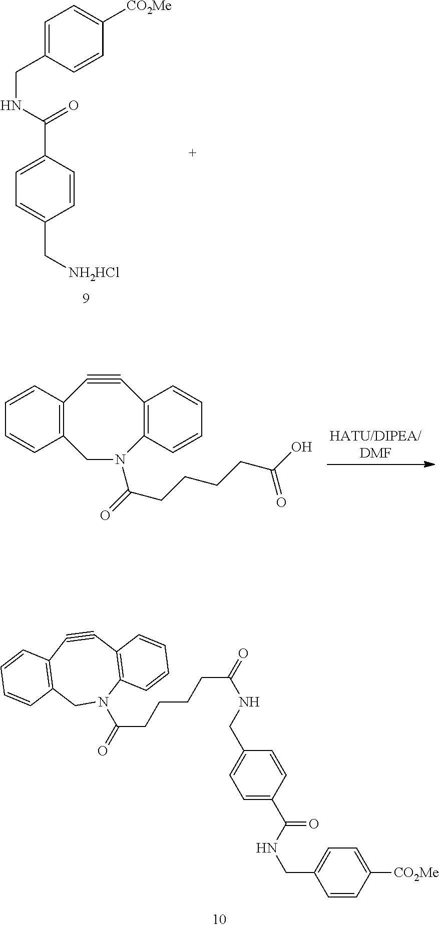

To a solution of DBCO-acid (0.1993 g, 0.5979 mmol), HATU (0.2726 g, 0.7175 mmol, 1.2 eq), DIPEA (0.1851 g, 0.249 mL, d=0.742 g/mL, 1.435 mmol, 2 eq) in DMF (0.3 mL) was added amine 9 (0.2 g, 0.5979 mmol)/DMF(0.3 mL), the reaction mixture was stirred at room temperature. After confirming by LC-MS that all starting material was consumed, the reaction mixture was concentrated under lyo, was loaded on to a silica column and purified (MPA: DCM, MPB: 20% MeOH in DCM, 0-30% ramp in 30 min) to afford the product 10. Yield: 0.3297 g, 90%. [M+H] calculated for C38H36N3O5: 614.26, found: 614.51.

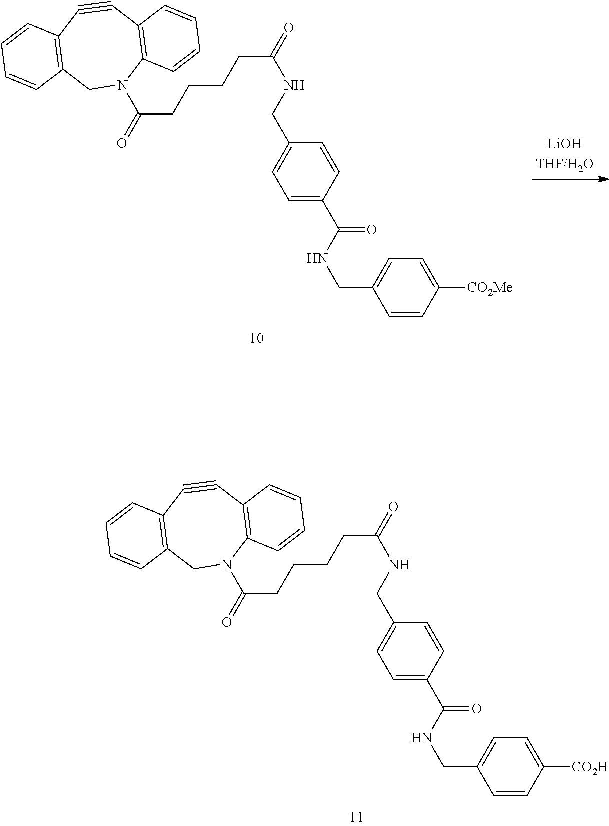 [SMILES: COC(=O)c1ccc(CNC(=O)c2ccc(CNC(=O)CCCCC(=O)N3Cc4ccccc4C#Cc4ccccc43)cc2)cc1 ; O=C(CCCCC(=O)N1Cc2ccccc2C#Cc2ccccc21)NCc1ccc(C(=O)NCc2ccc(C(=O)O)cc2)cc1]

To a solution of compound 10 in THE/water (4 mL, 1:1 v/v) was added LiOH (0.0387 g, 1.6117 mmol). The reaction was stirred at room temperature. After confirming by LC-MS that all starting material was consumed, HCl (1.6 mmol, 4M, 0.4 mL) in dioxane was added to neutralize the base. The reaction mixture was concentrated by lyophilization, was loaded on to a silica column and purified (MPA: DCM, MPB: 10% MeOH in DCM, 0-50% ramp in 30 min) to afford the product 11. Yield: 0.2575 g, 80%. [M+H] calculated for C₃₇H₃₄N₃O₅: 600.25, found: 600.46.

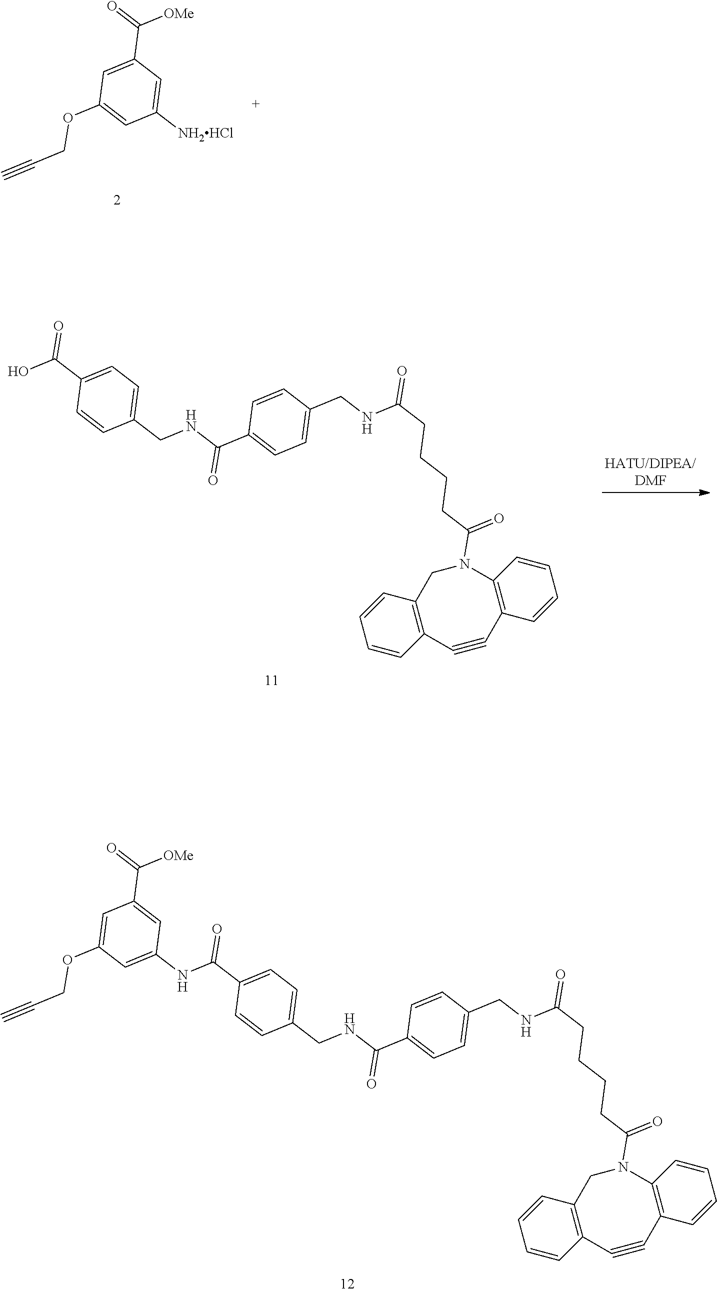

To a solution of acid 11 (0.1241 g, 0.2069 mmol), amine 2 (0.05 g, 0.2069 mmol), and DIPEA (0.0961 g, 0.129 mL, 0.7448 mmol, 3 eq, d=0.742 g/mL) in DMF (1.5 mL) was added HATU (0.0943 g, 0.2483 mmol)/DMF (0.5 mL). The reaction mixture was stirred at room temperature. Upon consumption of the starting material, the reaction mixture was concentrated under reduced pressure. After DMF was removed, the crude was dissolved into 5 mL DCM and loaded on a column with silica gel as the stationary phase. (MPA: DCM; MPB: 20% MeOH/DCM; 0-100% ramp in 30 min), Yield: 0.1109 g, 68%. [M+H] calculated for C₄₈H₄₃N₄O₇: 787.31, found: 787.44.

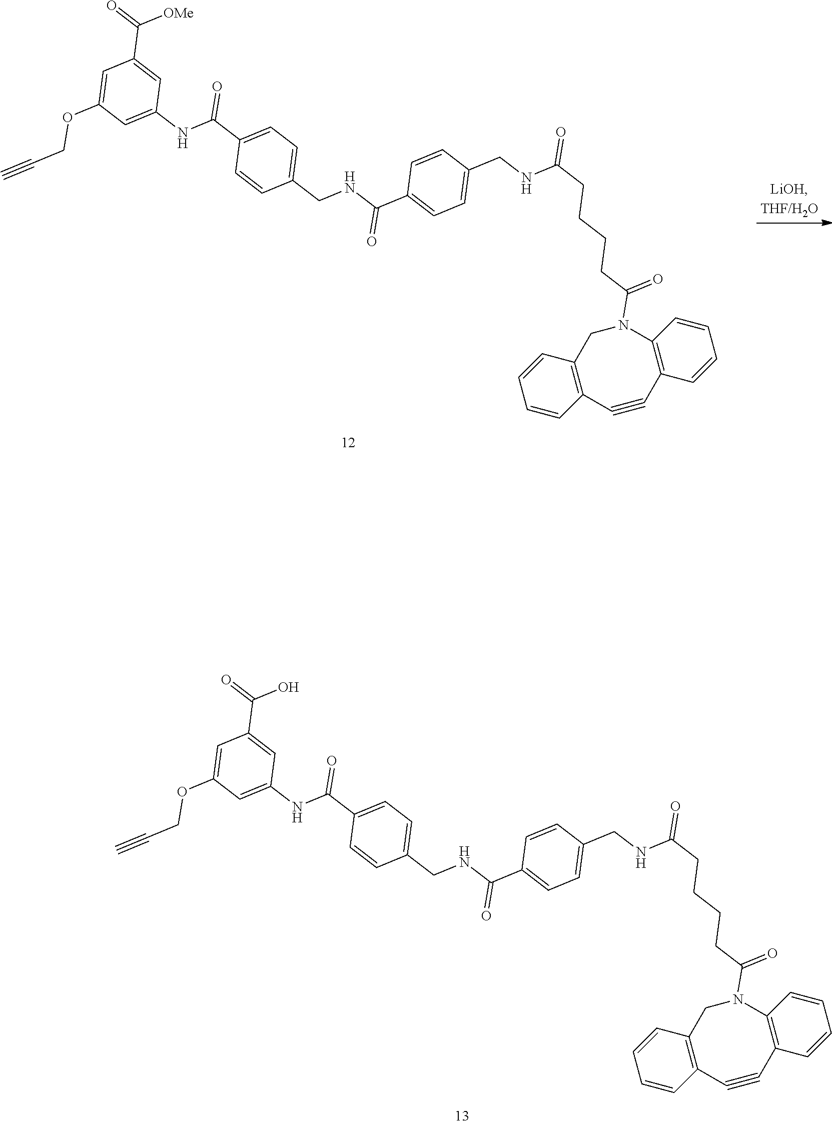 [SMILES: C#CCOc1cc(NC(=O)c2ccc(CNC(=O)c3ccc(CNC(=O)CCCCC(=O)N4Cc5ccccc5C#Cc5ccccc54)cc3)cc2)cc(C(=O)OC)c1 ; C#CCOc1cc(NC(=O)c2ccc(CNC(=O)c3ccc(CNC(=O)CCCCC(=O)N4Cc5ccccc5C#Cc5ccccc54)cc3)cc2)cc(C(=O)O)c1]

To a solution of DBCO-ester 12 in THE/water (1 mL, 1:1 v/v) was added LiOH (0.0169 g, 0.7047 mmol). The reaction was stirred at room temperature overnight. Upon the full completion of the starting material, the residue was neutralized by HCl (aq) and concentrated under reduced pressure. Purification by CombiFlash® afforded the final product 13. (MPA: DCM; MPB: 20% MeOH/DCM; 0-100% ramp in 30 min), Yield: 0.0321 g, solid, 29%. [M+H] calculated for C₄₇H₄₁N₄O₇: 773.30, found: 773.49.

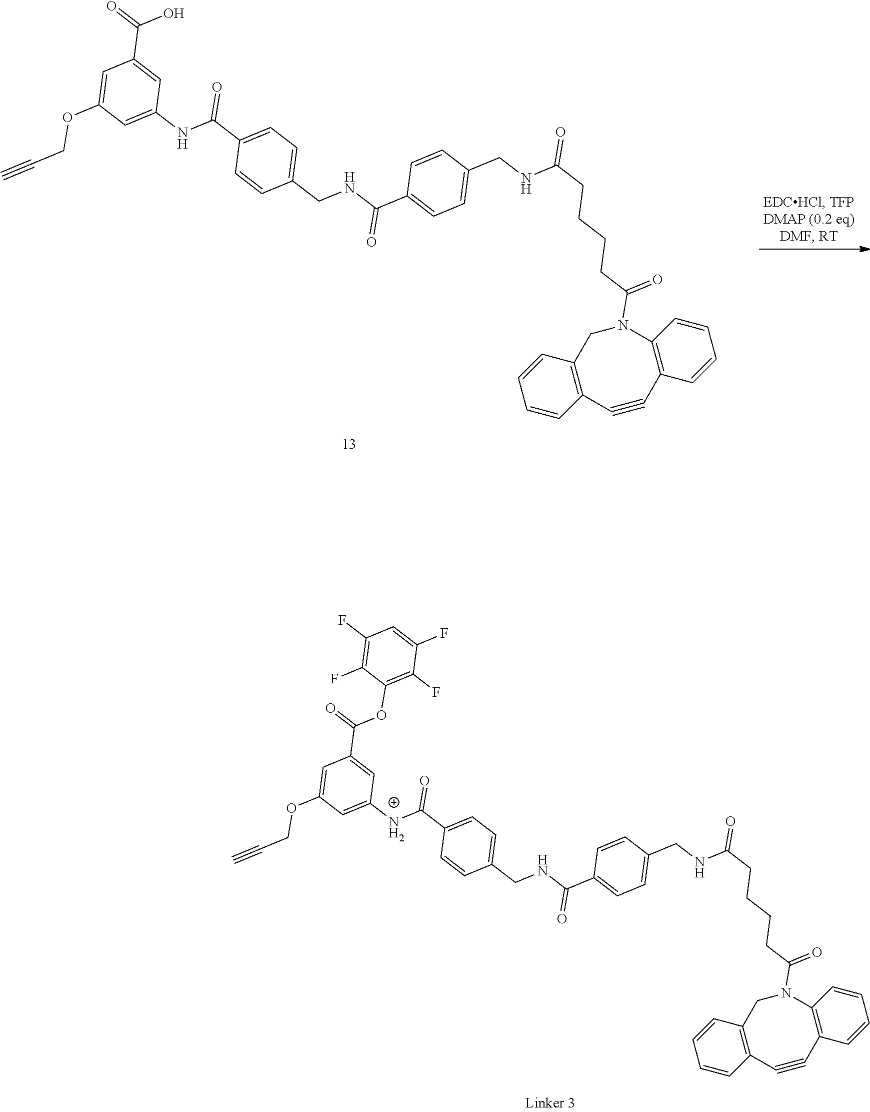 [SMILES: C#CCOc1cc(NC(=O)c2ccc(CNC(=O)c3ccc(CNC(=O)CCCCC(=O)N4Cc5ccccc5C#Cc5ccccc54)cc3)cc2)cc(C(=O)O)c1 ; C#CCOc1cc([NH2+]C(=O)c2ccc(CNC(=O)c3ccc(CNC(=O)CCCCC(=O)N4Cc5ccccc5C#Cc5ccccc54)cc3)cc2)cc(C(=O)Oc2c(F)c(F)cc(F)c2F)c1]

To a solution of acid 13 (0.0321 g, 0.0415 mmol), TFP (0.0103 g, 1.5 eq, 0.0623 mmol), DMAP (3 mg, 0.0249 mmol) in DMF (0.5 mL) was added EDC·HCl(0.0239 g, 0.1246 mmol). The reaction mixture was stirred at room temperature. Upon consumption of the starting material, the reaction mixture was concentrated under reduced pressure. After DMF was removed, the crude was dissolved into 5 mL DCM and loaded on a column with silica gel as the stationary phase. (MPA: DCM; MPB: 20% MeOH/DCM; 0-100% ramp in 30 min). Yield 20 mg, oil, 50%. [M+H] calculated for C₅₃H₄₁F₄N4O₇: 921.29, found: 921.85.

### Synthesis of Linker 4

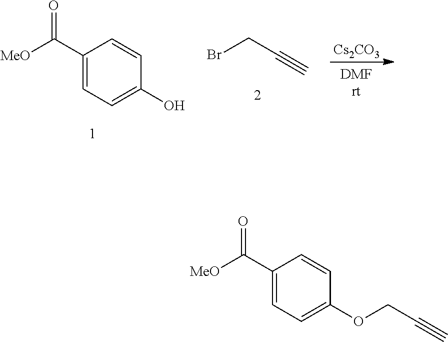

To a solution of compound 1 (3.00 g) in DMF was added Cs₂CO₃ (7.71 g) at rt. Compound 2 (1.85 mL) was then added slowly. Reaction was stirred overnight under N2 (g). Approx. full conversion to desired product by LC-MS was then confirmed. The reaction mixture was quenched with NaHCO₃(10 mL). The product was extracted with EtOAc (5×10 mL) and then washed with water (3×8 mL) and brine (8 mL). The combined organic phase was dried over Na₂SO₄, filtered, and concentrated. The residue was purified by CombiFlash using silica gel as the stationary phase with a gradient of hex to EtOAc (0-30%), in which product eluted at 14% B. The product was concentrated under vacuum to provide a white solid. LC-MS: calculated [M+H]+ 191.06 m/z, observed 191.23 m/z.

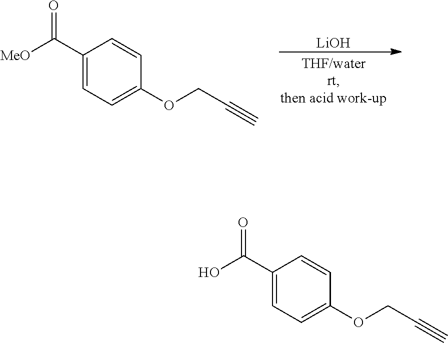

To a solution of compound 1 (2.87 g) in 1:1 THE/water was added LiOH (1.08 g) at rt under normal atmosphere. The reaction was stirred until full conversion was observed by LC-MS. Residual starting material was extracted via EtOAc, and then aqueous phase was acidifed with 6 N HCl to a pH of −3. Product crashed out as white solid and was filtered over vacuum and washed with water. Due to its wet/sticky nature, solvent was required to transfer the solid to a round bottom flask; material was transferred via MeOH and DCM. Due to poor solvation in either and the combination, material was not able to be dried over Na₂SO₄ and was correspondingly merely concentrated under vacuum to provide a white, fluffy crystalline solid. No isolation was necessary. LC-MS: calculated [M+H]+ 177.05 m/z, observed 177.19

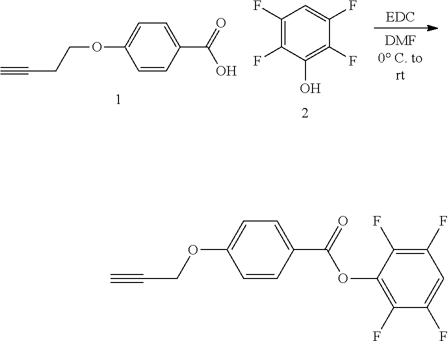

To a solution of compounds 1 (1.00 g) and 2 (1.04 g) in DMF (10.0 mL) under N2(g) was added EDC (1.20 g) at rt. The reaction mixture was allowed to stir until full conversion was observed by LC-MS. Due to an inability to successfully observe product after overnight stirring, reaction mixture was quenched with NaHCO₃, in which crash-out followed. Precipitate was confirmed to contain starting materials via LC-MS and was filtered over vacuum, attempted to be resuspended in MeOH/DCM, and then concentrated under vacuum. Mixture was then resolvated in DMF, dried over Na₂SO₄, and filtered over vacuum, rinsing with DMF. EDC was readded to filtrate (reaction mixture), and mixture was allowed to stir overnight at rt. The reaction mixture was directly concentrated and azeotroped with McOH and PhMe for isolation. The residue was purified by CombiFlash® using silica gel as the stationary phase and was eluted with a gradient of DCM to 20% of MeOH/DCM (0-15% B). Product eluted at 0% B to provide a white solid. LC-MS: calculated [M+H]+ 325.04 m/z, observed 325.35 m/z.

### Synthesis of Linker 7

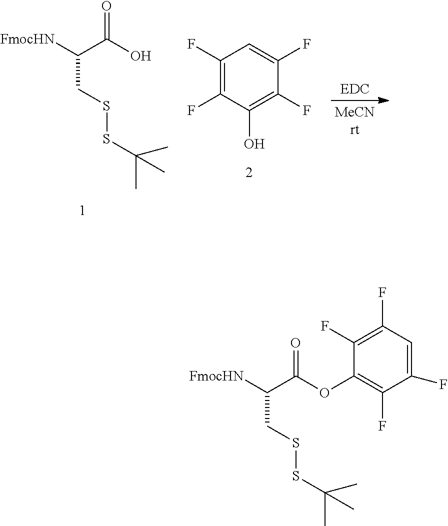

To a solution of compounds 1 (0.300 g) and 2 (0.231 g) in DMF was added EDC (0.160 g) under ambient conditions. The reaction was stirred for 2 h until full conversion was observed by LC-MS. The reaction mixture was then concentrated for isolation. The residue was purified by CombiFlash® using silica gel as the stationary phase with a gradient of hexanes to EtOAc (0-30%), in which product eluted at 10% B. The product was concentrated under vacuum to provide a white oily residue. Yield: 0.329 g (81.6%.) LC-MS: calculated [M+H]+ 580.12 m/z, observed 580.56 m/z.

### Synthesis of Linker 8

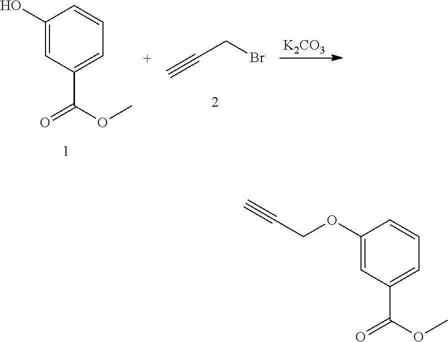

To a solution of compound 1 (500 mg, 3.286 mmol, 1.0 equiv.), and potassium carbonate (908 mg, 6.572 mmol, 2.0 equiv.) in anhydrous acetone (5 mL) was added compound 2 (0.549 mL, 4.929 mmol, 1.5 equiv.) at room temperature. The reaction was kept at 50° C. for 3 hrs. The reaction was quenched with saturated sodium bicarbonate solution (5 mL). The aqueous phase was extracted with ethyl acetate (3×5 mL). The organic phase was combined, dried over Na₂SO₄, and concentrated. The product was purified by CombiFlash® and was eluted with 5-10% ethyl acetate in hexane. LC-MS: calculated [M+H]+ 191.06, found 191.19.

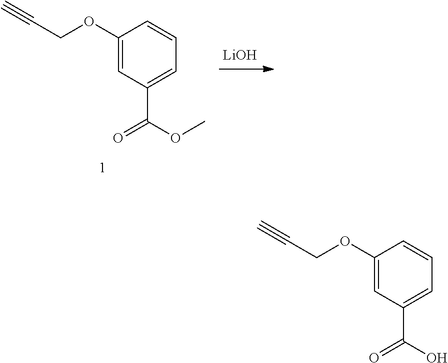

To a solution of compound 1 (584 mg, 3.070 mmol, 1.0 equiv.) in THE (5 mL) and water (5 mL) was added lithium hydroxide (220 mg, 9.211 mmol, 3.0 equiv.) at room temperature. The reaction was kept at 40° C. for 1 hr. The reaction was quenched with HCl solution and the pH was adjusted to 3.0. The aqueous phase was extracted with ethyl acetate (3×10 mL). The organic phase was combined, dried over Na₂SO₄, and concentrated. The product was used directly without further purification. LC-MS: calculated [M+H]+ 177.17, found 177.37.

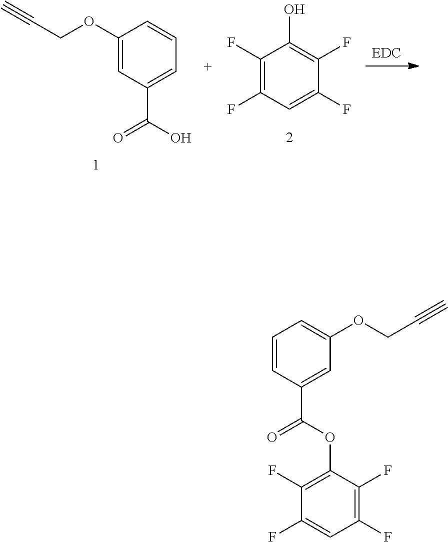 [SMILES: C#CCOc1cccc(C(=O)O)c1 ; Oc1c(F)c(F)cc(F)c1F ; C#CCOc1cccc(C(=O)Oc2c(F)c(F)cc(F)c2F)c1]

To a solution of compound 1 (185 mg, 1.050 mmol, 1.0 equiv.), compound 2 (218 mg, 1.312 mmol, 1.25 equiv.) in anhydrous DMF (2 mL) was added EDC HCl (251 mg, 1.312 mmol, 1.25 equiv.) at room temperature. The reaction was kept at room temperature for 2 hrs. The reaction was quenched with saturated sodium bicarbonate solution (5 mL). The aqueous phase was extracted with ethyl acetate (3×5 mL). The organic phase was combined, dried over Na₂SO₄, and concentrated. The product was purified by CombiFlash® and was eluted with 5-10% ethyl acetate in hexane. LC-MS: calculated [M+H]+ 325.04, found 325.26.

### Synthesis of Linker 9

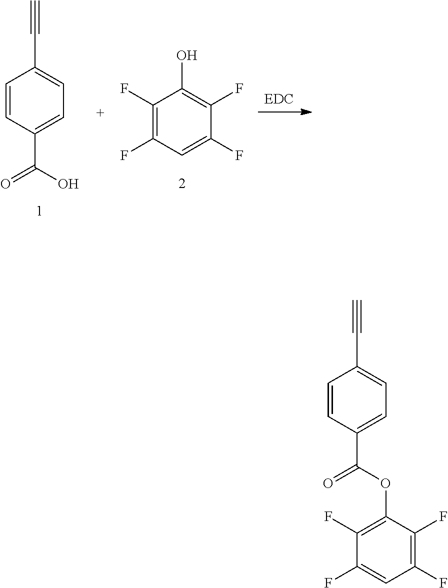 [SMILES: C#Cc1ccc(C(=O)O)cc1 ; Oc1c(F)c(F)cc(F)c1F ; C#Cc1ccc(C(=O)Oc2c(F)c(F)cc(F)c2F)cc1]

To a solution of compound 1 (200 mg, 1.368 mmol, 1.0 equiv.), compound 2 (284 mg, 1.710 mmol, 1.25 equiv.) in anhydrous DMF (2 mL) was added EDC HCl (327 mg, 1.710 mmol, 1.25 equiv.) at room temperature. The reaction was kept at room temperature for 2 hrs. The reaction was quenched with saturated sodium bicarbonate solution (5 mL). The aqueous phase was extracted with ethyl acetate (3×5 mL). The organic phase was combined, dried over Na₂SO₄, and concentrated. The product was purified by CombiFlash® and was eluted with 5-10% ethyl acetate in hexane. LC-MS: calculated [M+H]+ 295.03, found 294.69.

### Synthesis of Linker 10

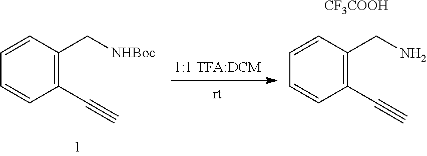

To a solution of compound 1 (0.200 g) in DCM was added TFA (1.99 mL) at room temperature. The reaction was stirred for 1 h under ambient conditions until full conversion was observed by LC-MS. The reaction mixture was azeotroped with PhMe and concentrated under vacuum to provide a brown oil. Yield: 0.309 g (146%.) LC-MS: calculated [M+H]+ 132.07 m/z, observed 132.10 m/z.

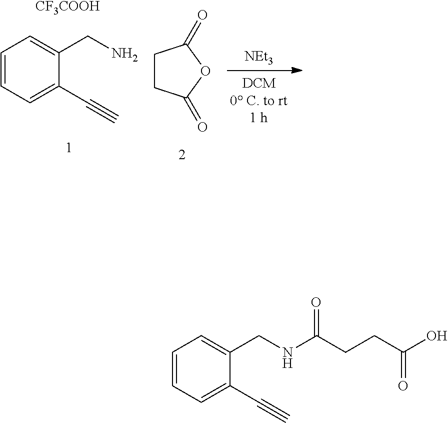

To a solution of compound 1 (0.212 g) in DCM was added 2 (0.0865 g) at 0° C. The mixture was stirred for 1.5 h and then warmed to room temperature to stir. After 0.5 h, NEt₃ was added, and within 0.5 h, full conversion was confirmed by LC-MS. The reaction mixture was concentrated immediately for isolation. The residue was purified by CombiFlash® using silica gel as the stationary phase and was eluted with a gradient of DCM to 20% MeOH in DCM (0-25% B). Product eluted at 9% B. Concentration provided a purple solid. Yield: 0.171 g (85.5%.) LC-MS: calculated [M+H]+ 232.09 m/z, observed 232.28 m/z.

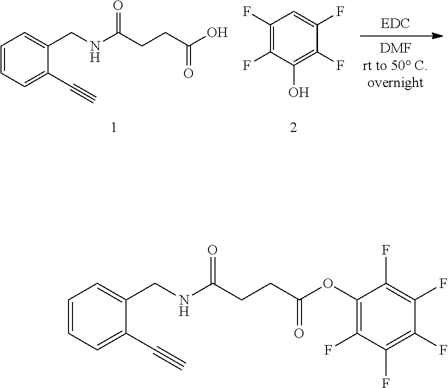

To a solution of compounds 1 (0.0400 g) and 2 (0.0316 g) in DMF was added EDC (0.0398 g) at room temperature. The reaction mixture was stirred for 1 h until full conversion was observed by LC-MS. After 1 h, full conversion was observed by LC-MS. The reaction mixture was quenched with NaHCO₃(15 mL). The product was extracted with EtOAc (3×8 mL) and washed with water (3×8 mL). The combined organic phase was dried over Na₂SO₄, filtered, and concentrated. The residue was purified by CombiFlash® using silica gel as the stationary phase and was eluted with a gradient of DCM to 20% of MeOH/DCM (0-25% B). Product eluted at 6% B to provide a white solid. Yield: 0.0255 g (38.9%.) LC-MS: calculated [M+H]+ 380.08 m/z, observed 380.35 m/z.

### Example 3. Synthesis of Targeting Ligands

### Synthesis of Structure 1b ((14S,17S)-1-azido-14-(5-((4-methylpyridin-2-yl)amino)pentanamido)-17-(4-(naphthalen-1-yl)phenyl)-15-oxo-3,6,9,12-tetraoxa-16-azanonadecan-19-oic acid)

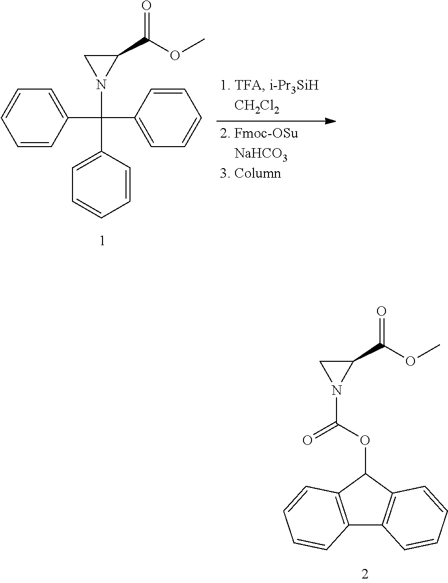 [SMILES: COC(=O)[C@@H]1CN1C(c1ccccc1)(c1ccccc1)c1ccccc1 ; COC(=O)[C@@H]1CN1C(=O)OC1c2ccccc2-c2ccccc21]

Compound 1 (Methyl (S)-(−)-1-tritylaziridine-2-carboxylate (4.204 g, 12.24 mmol, 1.0 equiv.) and triisopropylsilane (3.877 g, 5.02 mL, 24.48 mmol, 2 equiv.) were dissolved in DCM (40 mL), the solution was cooled to 0° C., and then TFA (8.5 eq) was added dropwise. The solution remained for 1 hour at 0° C. The reaction was monitored by TLC Hexane: Ethyl Acetate (8: 2). The solution was dried to yield a mixture of white precipitate and light yellow oil. Hexanes (40 mL) were added and heated gently over heat gun until all white precipitate dissolved. The addition of hexanes resulted in two layers, a clear upper layer and an oil layer. The hexane layer was poured off and the oil layer was retained. The hexanes addition was repeated and once again poured off. The oil was allowed to dry. The aziridine (1.06 g, 10.5 mmol) was dissolved in THF/H₂O (2/1) 60 mL total. Fmoc-OSu (5.312 g, 15.75 mmol, 1.5 eq) and NaHCO₃(2.646 g, 31.5 mmol, 3 eq to keep pH=8.5) were added to the mixture at room temp and allowed to react overnight. The reaction was monitored by TLC, Hexane:Ethyl Acetate 8:2. The mixture was concentrated until all the THF was removed, then diluted with ethyl acetate (350 mL) and H₂O (25 mL). The layers were separated, and the organics washed with H₂O (40 mL). The organics were then washed with pH 3-4 water (2×40 mL), then H₂O (40 mL), then saturated aq. NaCl solution (40 mL). The organic phase was dried over Na₂SO₄, filtered, and concentrated. The product was purified on silica column 10%-20% ethyl acetate in hexanes.

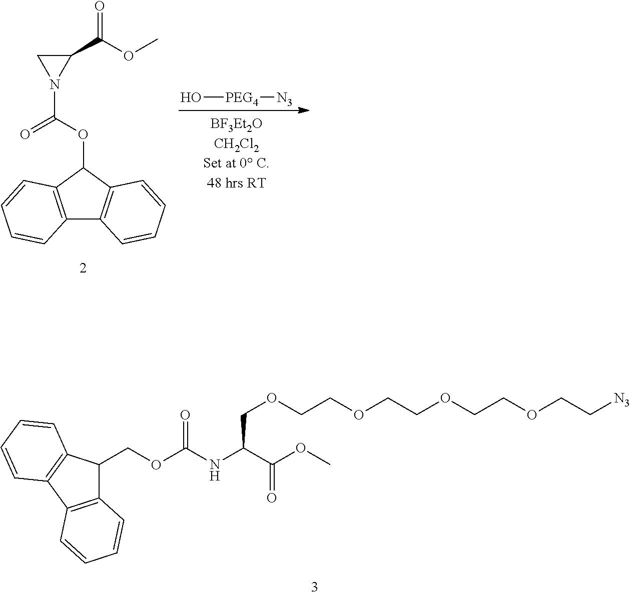

Compound 2 (Fmoc-aziridine) (1.46 g, 4.52 mmol) and HO-PEG₄-N₃ (1.983 g, 9.04 mmol, 2 eq) were dissolved in DCM. The mixture was cooled to 0° C. Boron trifluoride diethyl etherate (12 drops) was added dropwise. The mixture was stirred at RT for 48 hours. The reaction was monitored by TLC, DCM with 5% MeOH. The reaction was quenched with NH₄Cl saturated solution (5 mL), diluted with DCM (60 mL) and washed with H₂O (3×20 mL), saturated aq. NaCl solution (20 mL), dried over Na₂SO₄, filtered, and concentrated. The product was purified on a silica column, 40%-60% ethyl acetate in hexanes.

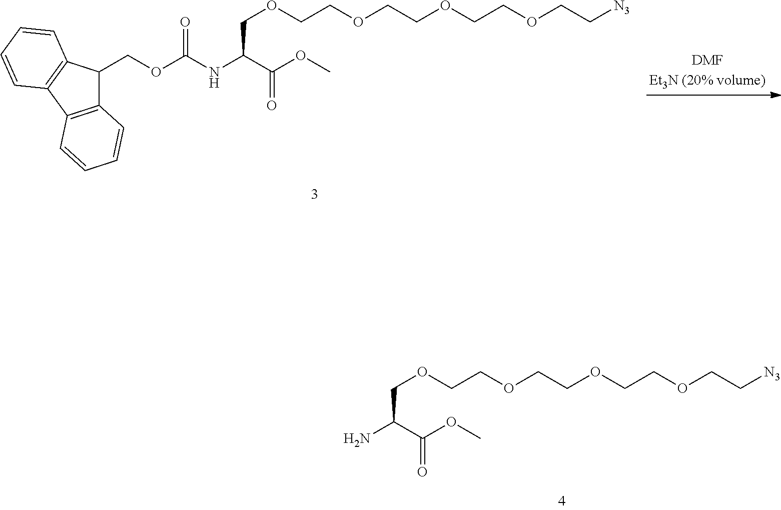 [SMILES: COC(=O)[C@H](COCCOCCOCCOCCN=[N+]=[N-])NC(=O)OCC1c2ccccc2-c2ccccc21 ; COC(=O)[C@@H](N)COCCOCCOCCOCCN=[N+]=[N-]]

Compound 3 was dissolved in a solution of 20% triethylamine in DMF. The reaction was monitored by TLC. The product was concentrated.

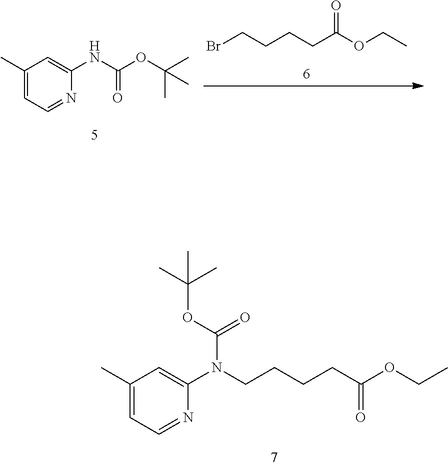

Compound 5 (tert-Butyl(4-methylpyridin-2-yl)carbamate) (0.501 g, 2.406 mmol, 1.0 equiv.) was dissolved in DMF (17 mL). To the mixture was added NaH (0.116 mg, 3.01 mmol, 1.25 eq, 60% dispersion in mineral oil) at room temperature. The mixture stirred for 10 min, then ethyl 5-bromovalerate (0.798 g, 3.82 mmol, 0.604 mL) was added. After 3 hours the reaction was quenched with ethanol (18 mL) and concentrated. The product was dissolved in DCM (50 mL) and washed with saturated aq. NaCl solution (50 mL), dried over Na₂SO₄, filtered and concentrated. The product was purified on silica column, gradient 0-5% methanol in DCM.

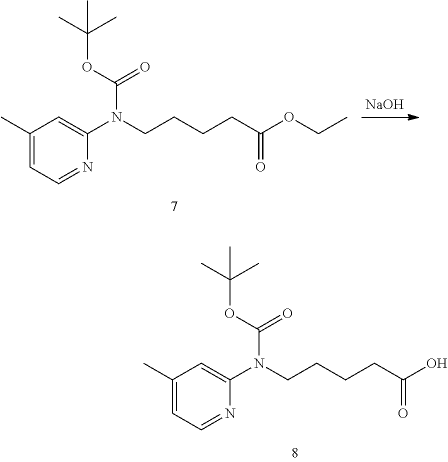

Compound 7 (0.80 g, 2.378 mmol) was dissolved in 100 mL of acetone: 0.1 M NaOH (1:1), and the reaction was monitored by TLC (5% ethyl acetate in hexane). The organics were concentrated, and the mixture was acidified to pH 3-4 with 0.3 M citric acid (40 mL). The product was extracted with DCM (3×75 mL). The organics were pooled, dried over Na₂SO₄, filtered and concentrated. The product was used without further purification.

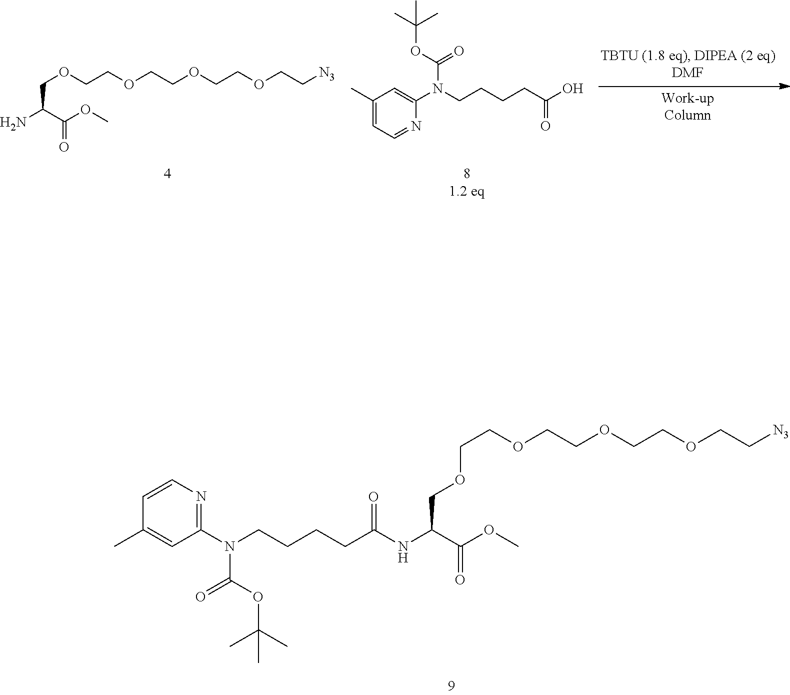

Compound 4 was dissolved (0.340 g, 1.104 mmol) in DMF (10 mL). To the solution was added TBTU (0.531 g, 1.655 mmol) and diisopropylethylamine (0.320 mL, 1.839 mmol). Then compound 8 was added (0.295 g, 0.9197 mmol). The reaction was monitored by LC-MS and TLC (DCM with 5% MeOH). The reaction was complete in 2 hours. The product was concentrated and dissolved in ethyl acetate (150 mL), and washed with pH 3-4 H₂O (2×12 mL). Then the product was washed with H₂O (2×12 mL), saturated aq. NaHCO₃ solution (12 mL), then saturated aq. NaCl solution (12 mL). The organic phase was dried over Na₂SO₄, filtered and concentrated. The product was purified on silica column, hexanes 20% in ethyl acetate to 100% ethyl acetate.

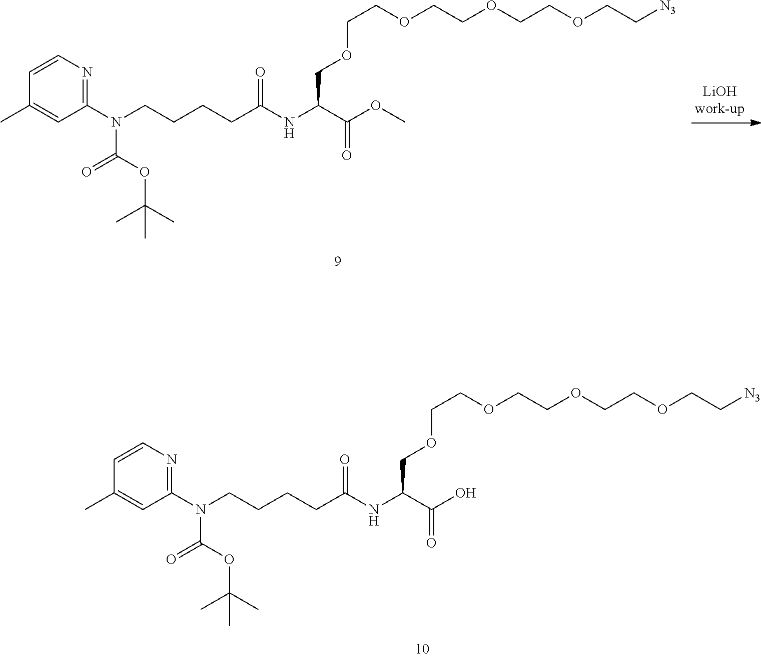 [SMILES: COC(=O)[C@H](COCCOCCOCCOCCN=[N+]=[N-])NC(=O)CCCCN(C(=O)OC(C)(C)C)c1cc(C)ccn1 ; Cc1ccnc(N(CCCCC(=O)N[C@@H](COCCOCCOCCOCCN=[N+]=[N-])C(=O)O)C(=O)OC(C)(C)C)c1]

Compound 9 was dissolved (0.330 g, 0.540 mmol) in 10 mL of MeOH:dioxane [1:1] and 1 M LiOH solution (10 mL) The mixture was stirred at room temperature for 2 hr, monitored by LC-MS and TLC (EtOAc). The organics were concentrated away, and the mixture was diluted with H₂O (5 mL) and acidified to pH 4. The product was extracted with ethyl acetate (2×50 mL). The organics were pooled, washed with saturated aq. NaCl solution (10 mL), dried over Na₂SO₄, filtered and concentrated. The product was used without further purification.

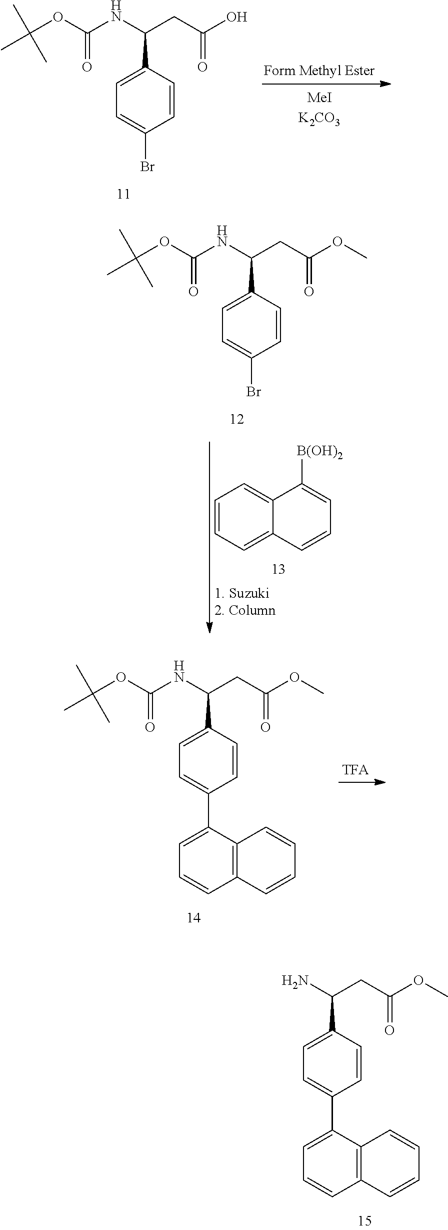 [SMILES: CC(C)(C)OC(=O)N[C@@H](CC(=O)O)c1ccc(Br)cc1 ; COC(=O)C[C@H](NC(=O)OC(C)(C)C)c1ccc(Br)cc1 ; OB(O)c1cccc2ccccc12 ; COC(=O)C[C@H](NC(=O)OC(C)(C)C)c1ccc(-c2cccc3ccccc23)cc1 ; COC(=O)C[C@H](N)c1ccc(-c2cccc3ccccc23)cc1]

Compound 11((S)-3-(4-Bromophenyl)-3-((tert-butoxycarbonyl)amino)-propionic acid) (2.0 g, 5.81 mmol) was dissolved in DMF (40 mL). To the mixture was added K₂CO₃ (1.2 g, 8.72 mmol). Then iodomethane (1.65 g, 11.62 mmol, 0.72 mL) was added. The reaction was monitored by TLC (hexane: ethyl acetate (7:3)). Upon completion, the mixture was cooled to 0° C. and H₂O (20 mL) and MTBE (40 mL) were added. The product was extracted with MTBE (4×40 mL). The combined organic phase was washed with saturated aq. NaHCO₃(40 mL) then H₂O (4×40 mL). The mixture was dried over Na₂SO₄, filtered and concentrated.

To dried product compound 12 (1.0 g, 2.7915 mmol) was added compound 13 (1-Naphthalene Boronic Acid (0.960 g, 5.583 mmol, 2 eq)). To the mixture was added [1,1′-Bis(diphenylphosphino)ferrocene]dichloropalladium(II) or Pd(dppf)Cl₂(0.0817 g, 0.1117 mmol, 0.4 eq) along with Na₂CO₃ (0.888 g, 8.375 mmol, 3 eq). Next, 1,4-dioxane (5 mL) and H₂O (0.2 mL) were added, and the mixture was stirred at 100° C. for 4 hr. The reaction was monitored by TLC (hexane: ethyl acetate (7:3)). The product was purified by silica chromatography, gradient 0% to 50% ethyl acetate in hexanes.

Compound 14 (0.200 g, 0.493 mmol) was dissolved in DCM (2.5 mL), then TFA (0.45 mL) was added. The reaction was monitored by TLC, (DCM: methanol (9: 1)). Upon completion, the reaction mixture was concentrated. The residue was dissolved in DCM (4 mL) and washed with saturated aq. NaHCO₃ solution (2×2 mL) then saturated aq. NaCl solution (2×2 mL). The organic phase was dried over Na₂SO₄, filtered and concentrated. The product was used without further purification.

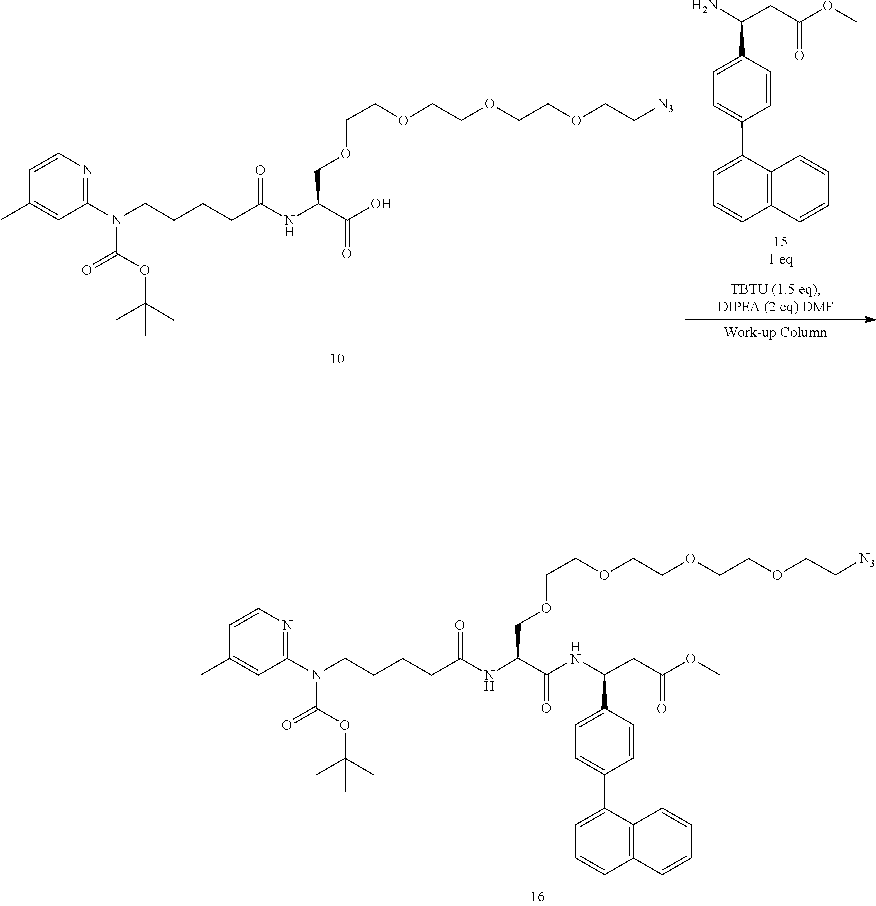 [SMILES: Cc1ccnc(N(CCCCC(=O)N[C@@H](COCCOCCOCCOCCN=[N+]=[N-])C(=O)O)C(=O)OC(C)(C)C)c1 ; COC(=O)C[C@H](N)c1ccc(-c2cccc3ccccc23)cc1 ; COC(=O)C[C@H](NC(=O)[C@H](COCCOCCOCCOCCN=[N+]=[N-])NC(=O)CCCCN(C(=O)OC(C)(C)C)c1cc(C)ccn1)c1ccc(-c2cccc3ccccc23)cc1]

Compound 10 (0.3224 g, 0.54 mmol) was dissolved in DMF (7 mL). To the mixture was added TBTU (0.236 g, 0.735 mmol) and diisopropylethylamine (0.170 mL, 0.98 mmol). Then compound 15 was added (0.1496 g, 0.49 mmol). The reaction was stirred at RT for 2 hours. The reaction was monitored by LC-MS. The mixture was concentrated, and the residue was dissolved in ethyl acetate (90 mL), and washed with pH 3-4 H₂O (3×10 mL). The product was washed with H₂O (2×10 mL), saturated aq. NaHCO₃ solution (10 mL), and then saturated aq. NaCl solution (1×10 mL). The organic phase was dried over Na₂SO₄, filtered and concentrated. The product was purified by silica chromatography using DCM, gradient to 5% MeOH.

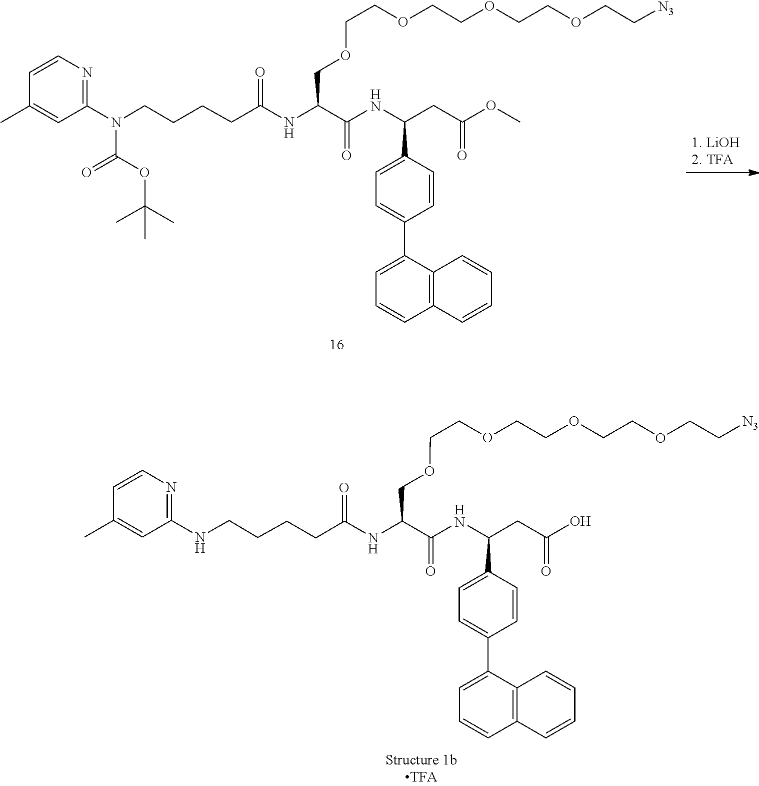 [SMILES: COC(=O)C[C@H](NC(=O)[C@H](COCCOCCOCCOCCN=[N+]=[N-])NC(=O)CCCCN(C(=O)OC(C)(C)C)c1cc(C)ccn1)c1ccc(-c2cccc3ccccc23)cc1 ; Cc1ccnc(NCCCCC(=O)N[C@@H](COCCOCCOCCOCCN=[N+]=[N-])C(=O)N[C@@H](CC(=O)O)c2ccc(-c3cccc4ccccc34)cc2)c1]

A Compound 16 was dissolved (0.250 g, 0.2828 mmol) in MeOH:dioxane [1:1](4 mL) and 1 M LiOH (4 mL) The mixture was stirred at RT for 2 hr. The organics were concentrated away, and the residue was diluted with H₂O (3 mL) and acidified to pH 4. The product was extracted with ethyl acetate (3×20 mL). The organics were pooled and washed with saturated aq. NaCl solution (10 mL). The product was dried over Na₂SO₄. The product was dissolved (0.200 g, 0.2299 mmol) in 2 mL DCM: TFA [25:75] and stirred at RT for 2 hours. To the mixture was added toluene (4 mL). The mixture was concentrated, then coevaporated with acetonitrile (2×4 mL). The product was purified by HPLC, gradient 35% ACN to 50% over 30 minutes, 0.1% TFA buffer.=>[M+H]+ calculated for C₄₁H₅₁N₇O₈: 769.90, found: 770.45; ¹H NMR (400 MHz, DMSO) δ 8.64 (d, 1H), 8.07 (d, 1H), 8.00 (d, 1H), 7.95 (d, 1H), 7.78 (t, 2H), 7.60-7.40 (m, 8H), 6.80 (s, 1H), 6.67 (d, 1H), 5.31 (q, 1H), 4.55 (m, 1H), 3.62-3.45 (m, 18H), 3.40 (t, 2H), 3.25 (m, 2H), 2.80 (dd, 2H), 2.30 (s, 3H), 2.20 (t, 2H), 1.55 (m, 4H).

### Synthesis of Structure 2b ((14S,17S)-1-azido-14-(4-((4-methylpyridin-2-yl)amino)butanamido)-17-(4-(naphthalen-1-yl)phenyl)-15-oxo-3,6,9,12-tetraoxa-16-azanonadecan-19-oic Acid)

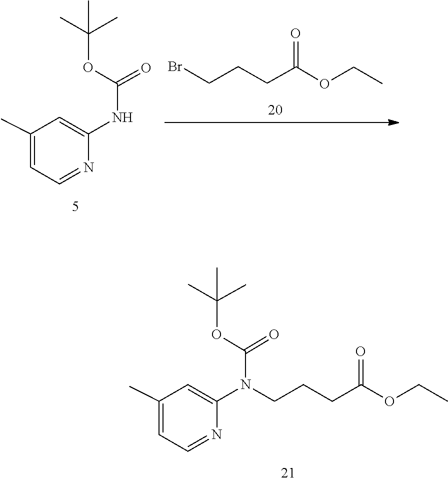

Compound 5 (tert-Butyl(4-methylpyridin-2-yl)carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

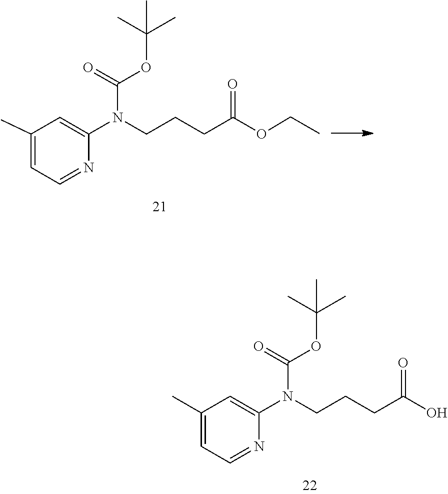

Compound 21 was dissolved (0.80 g, 2.378 mmol) in 100 mL of Acetone: 0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification

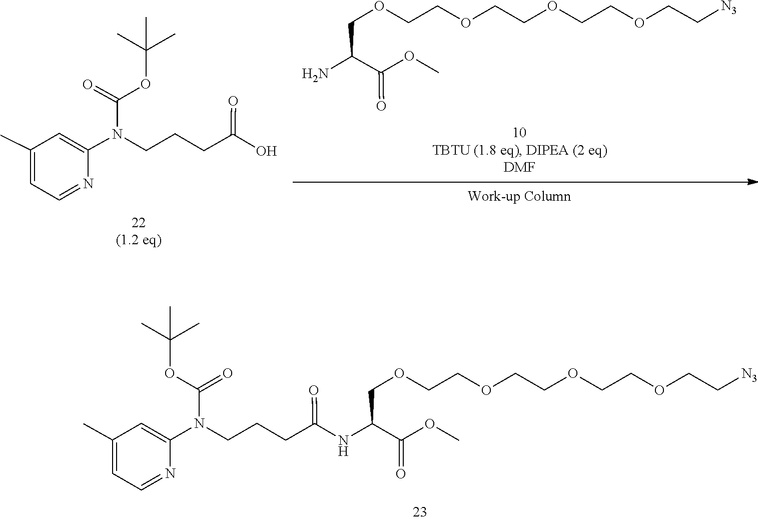 [SMILES: Cc1ccnc(N(CCCC(=O)O)C(=O)OC(C)(C)C)c1 ; COC(=O)[C@@H](N)COCCOCCOCCOCCN=[N+]=[N-] ; COC(=O)[C@H](COCCOCCOCCOCCN=[N+]=[N-])NC(=O)CCCN(C(=O)OC(C)(C)C)c1cc(C)ccn1]

Compound 22 was dissolved (0.340 g, 1.104 mmol) in DMF (10 mL). To the mixture was added TBTU (0.531 g, 1.655 mmol) and diisopropylethylamine (0.320 mL, 1.839 mmol). Then Compound 10 (0.295 g, 0.9197 mmol) was added. The reaction was monitored by LC-MS and TLC (DCM with 5% MeOH). The reaction was complete in 2 hr. The mixture was concentrated, dissolved in ethyl acetate (150 mL), and washed with pH 3-4 H₂O (2×12 mL). The mixture was then washed with H₂O (2×12 mL), saturated aq. NaHCO₃ solution (12 mL), then saturated aq. NaCl solution (12 mL). The organic phase was dried over Na₂SO₄, filtered and concentrated. The product was purified on silica column, Hexanes 20% in ethyl acetate to 100% ethyl acetate.

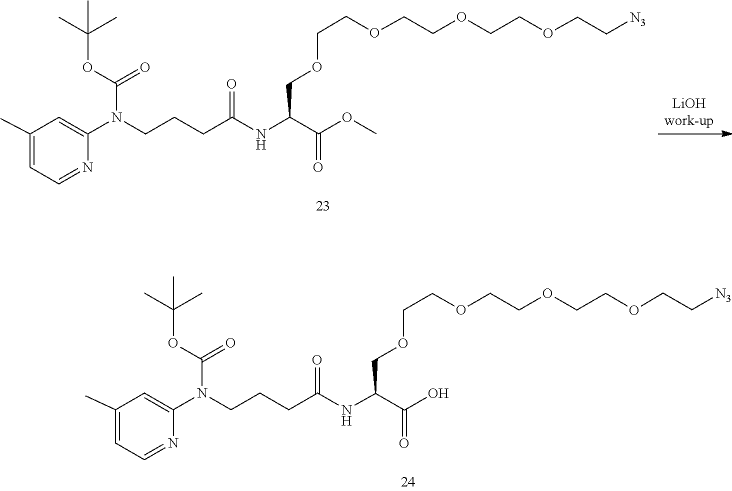 [SMILES: COC(=O)[C@H](COCCOCCOCCOCCN=[N+]=[N-])NC(=O)CCCN(C(=O)OC(C)(C)C)c1cc(C)ccn1 ; Cc1ccnc(N(CCCC(=O)N[C@@H](COCCOCCOCCOCCN=[N+]=[N-])C(=O)O)C(=O)OC(C)(C)C)c1]

Compound 23 was dissolved (0.330 g, 0.540 mmol) in 10 mL of MeOH: Dioxane [1:1] and 1 M LiOH (10 mL) The mixture was stirred at room temperature for 2 hours and monitored by LC-MS and TLC (100% EtOAc). The organics were concentrated, and the residue was diluted with H₂O (5 mL), and acidified to pH 4. The product was extracted with ethyl acetate (2×50 mL). The combined organic phase was washed with saturated aq. NaCl solution (1×10 mL). The organic phase was dried over Na₂SO₄, filtered and concentrated. The product was used without further purification.

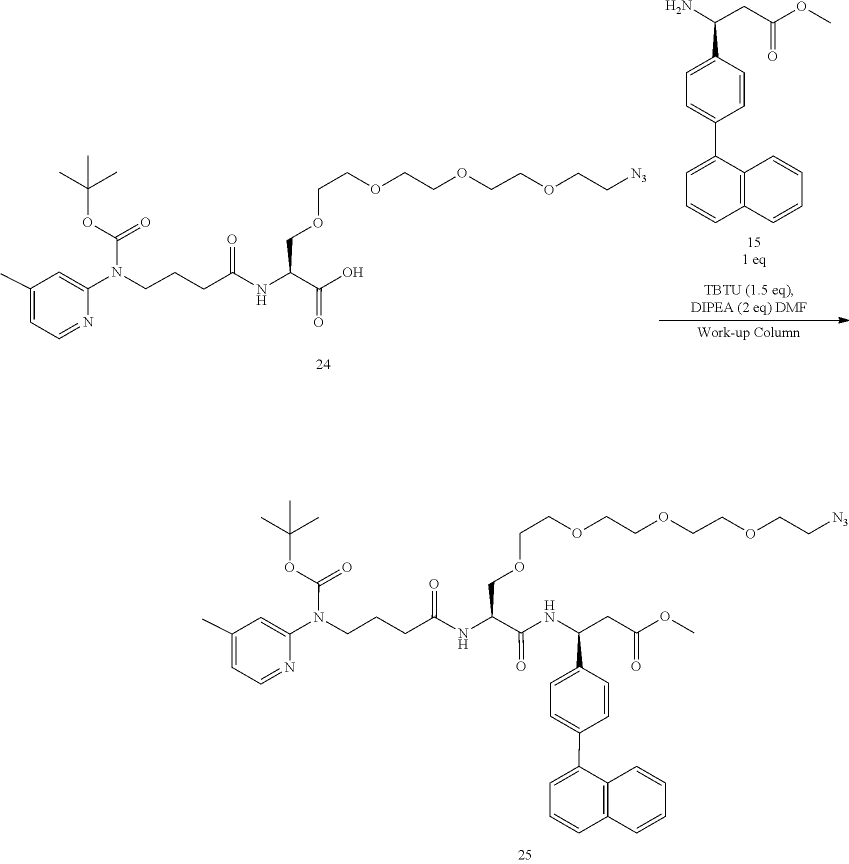 [SMILES: Cc1ccnc(N(CCCC(=O)N[C@@H](COCCOCCOCCOCCN=[N+]=[N-])C(=O)O)C(=O)OC(C)(C)C)c1 ; COC(=O)C[C@H](N)c1ccc(-c2cccc3ccccc23)cc1 ; COC(=O)C[C@H](NC(=O)[C@H](COCCOCCOCCOCCN=[N+]=[N-])NC(=O)CCCN(C(=O)OC(C)(C)C)c1cc(C)ccn1)c1ccc(-c2cccc3ccccc23)cc1]

Compound 24 was dissolved (0.3224 g, 0.54 mmol) in DMF (7 mL). To the mixture was added TBTU (0.236 g, 0.735 mmol) and diisopropylethylamine (0.170 mL, 0.98 mmol). Compound 15 was then added (0.1496 g, 0.49 mmol). The mixture was stirred at room temperature for 2 hours. The reaction was monitored by LC-MS. The mixture was concentrated, and the residue was dissolved in ethyl acetate (90 mL) and washed with pH 3-4 H₂O (3×10 mL). The concentrate was washed with H₂O (2×10 mL), saturated aq. NaHCO₃ solution (10 mL), and then saturated aq. NaCl solution (10 mL). The organic phase was dried over Na₂SO₄, filtered and concentrated. The product was purified on silica column, DCM, gradient to 5% MeOH.

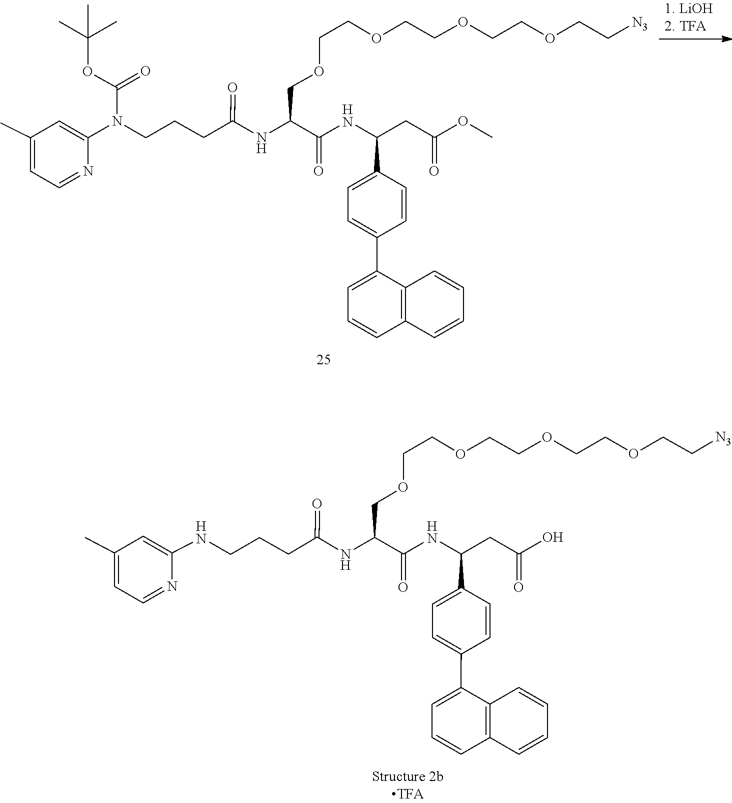 [SMILES: COC(=O)C[C@H](NC(=O)[C@H](COCCOCCOCCOCCN=[N+]=[N-])NC(=O)CCCN(C(=O)OC(C)(C)C)c1cc(C)ccn1)c1ccc(-c2cccc3ccccc23)cc1 ; Cc1ccnc(NCCCC(=O)N[C@@H](COCCOCCOCCOCCN=[N+]=[N-])C(=O)N[C@@H](CC(=O)O)c2ccc(-c3cccc4ccccc34)cc2)c1]

Compound 25 was dissolved (0.250 g, 0.2828 mmol) in MeOH: Dioxane [1:1] (4 mL) and 1 M LiOH (4 mL). The mixture was stirred at room temperature for 2 hr, monitored by LC-MS. The organics were concentrated, and the residue was diluted with H₂O (3 mL) and acidified to pH 4. The product was extracted with ethyl acetate (3×20 mL). The organics were pooled and washed with saturated aq. NaCl solution (1×10 mL). The organic phase was dried over Na₂SO₄ and concentrated. The residue was dissolved (0.200 g, 0.2299 mmol) in 2 mL DCM/TFA (25/75) and stirred at RT for 2 hours while monitored by LC-MS. Toluene (4 mL) was added, and the mixture was concentrated. Then acetonitrile (2×4 mL) was added, and the mixture was concentrated. The product was purified on HPLC, gradient 35% ACN to 50% over 30 minutes, 0.1% TFA buffer. [M+H]+ calculated for C₄₀H₄₉N₇O₈: 755.87, found: 756.32; ¹H NMR (400 MHz, DMSO) δ 8.64 (t, 1H), 8.17-8.10 (m, 1H), 8.00 (d, 1H), 7.95 (d, 1H), 7.80 (d, 1H), 7.75 (m, 1H), 7.60-7.40 (m, 8H), 6.8 (s, 1H), 6.67 (d, 1H), 5.31 (q, 1H), 4.55 (m, 1H), 3.62-3.45 (m, 18H), 3.40 (t, 2H), 3.25 (m, 2H), 2.80 (dd, 2H), 2.30 (s, 3H), 2.26 (t, 2H), 1.80 (m, 2H).

### Synthesis of Structure 5b, 5.1b, and 5.2b

### Structure 5b (3-(4-(2-(2-(2-azidoethoxy)ethoxy)ethoxy)-3,5-dichlorophenyl)-3-(2-(5-((4-methylpyridin-2-yl)amino)pentanamido)acetamido)propanoic acid)

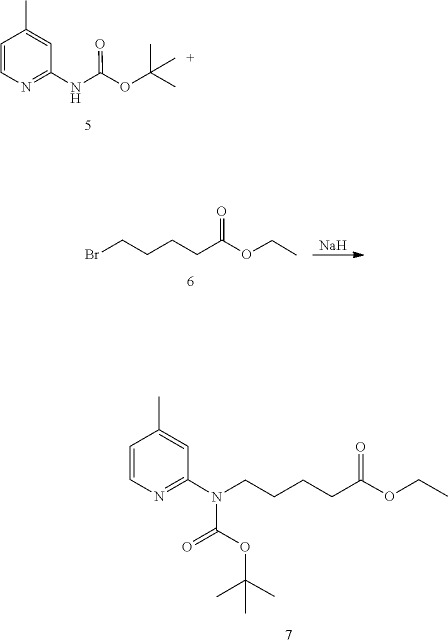

To a solution of Compound 5 (0.98 g, 4.70 mmol, 1 equiv.) in dry DMF (10 mL) was added NaH (0.226 g, 5.647 mmol, 1.2 equiv., 60% oil dispersion) portion-wise at 0° C. under N₂ atmosphere. The reaction mixture was kept at 0° C. for 30 min followed by the addition of compound 6 (1.18 mL, 5.647 mmol, 1.2 equiv.) at the same temperature. After additional stirring at 0° C. for 30 min the mixture was allowed to warm to room temperature. After stirring at room temperature for 1 hour, the reaction was quenched by saturated NH₄C₁ aqueous solution. The aqueous phase was extracted with ethyl acetate (3×20 mL) and the organic layer was combined, dried over Na₂SO₄, and concentrated. The product was separated by CombiFlash® using silica gel as the stationary phase. LC-MS: [M+H]+ 337.20, found 337.39.

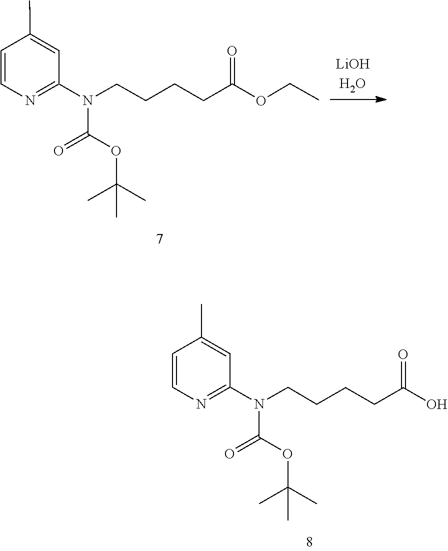 [SMILES: CCOC(=O)CCCCN(C(=O)OC(C)(C)C)c1cc(C)ccn1 ; Cc1ccnc(N(CCCCC(=O)O)C(=O)OC(C)(C)C)c1]

To a solution of compound 7 (1.347 g, 4.00 mmol, 1 equiv.) in THE (5 mL) and H₂O (5 mL) was added lithium hydroxide monohydrate (0.505 g, 12.01 mmol, 5 equiv.) portion-wise at 0° C. The reaction mixture was warmed to room temperature. After stirring at room temperature for 1 h, the reaction mixture was acidified by HCl (6 N) to pH 4.0. The aqueous phase was extracted with ethyl acetate (3×20 mL) and the organic layer was combined, dried over Na₂SO₄, and concentrated. LC-MS: [M+H]+ 309.17, found 309.39.

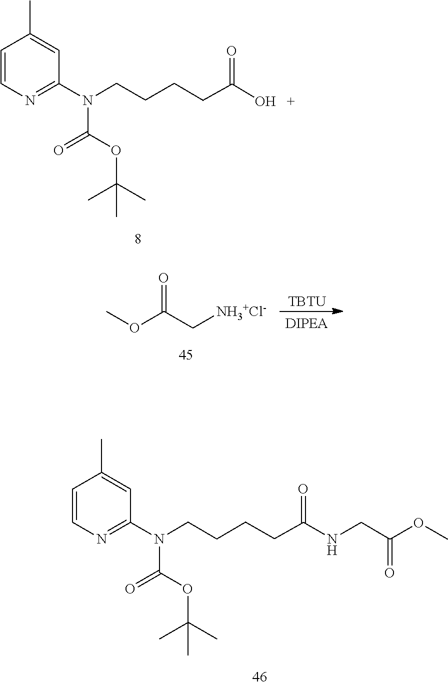

To a solution of Compound 8 (1.163 g, 3.77 mmol, 1 equiv.), Compound 45 (568 mg, 4.52 mmol, 1.2 equiv.), and TBTU (1.453 g, 4.52 mmol, 1.2 equiv.) in anhydrous DMF (10 mL) was added diisopropylethylamine (1.97 mL, 11.31 mmol, 3 equiv.) at 0° C. The reaction mixture was warmed to room temperature and stirred 3 hours. The reaction was quenched by saturated NaHCO₃ solution (20 mL). The aqueous layer was extracted with ethyl acetate (3×10 mL), and the organic phase was combined, dried over anhydrous Na₂SO₄, and concentrated. The product was separated by CombiFlash® using silica gel as the stationary phase. LC-MS: calculated [M+H]+ 380.21, found 380.51.

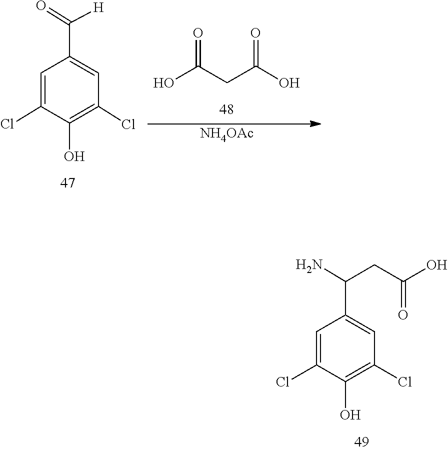 [SMILES: O=Cc1cc(Cl)c(O)c(Cl)c1 ; O=C(O)CC(=O)O ; NC(CC(=O)O)c1cc(Cl)c(O)c(Cl)c1 ; "NH4OAc"]

To a solution of compound 47 (1.0 g, 5.23 mmol, 1 equiv.) and malonic acid (1.09 g, 10.47 mmol, 2 equiv.) in ethanol (10 mL) was added ammonium acetate (0.807 mg, 10.47 mmol, 2.0 equiv.) at room temperature. The reaction mixture was stirred at reflux overnight. The solid was filtered and washed with cold ethanol. The product was used directly for further steps without further purification. LC-MS: calculated [M+H]+ 250.00, found 250.16.

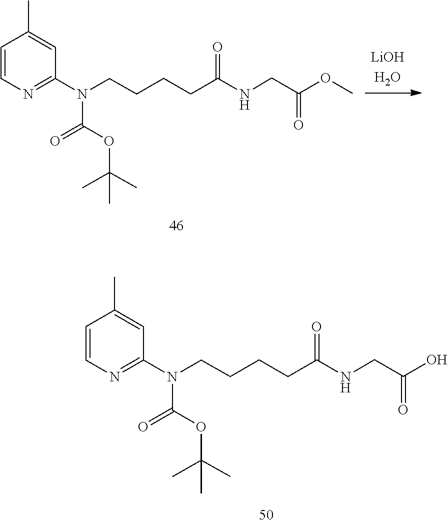 [SMILES: COC(=O)CNC(=O)CCCCN(C(=O)OC(C)(C)C)c1cc(C)ccn1 ; Cc1ccnc(N(CCCCC(=O)NCC(=O)O)C(=O)OC(C)(C)C)c1]

To a solution of compound 46 (1.412 g, 3.72 mmol, 1 equiv.) in THE (5 mL) and H₂O (5 mL) was added lithium hydroxide monohydrate (0.469 g, 11.16 mmol, 3 equiv.) portion-wise at 0° C. The reaction mixture was warmed to room temperature. After stirring at room temperature for 3 hours, the reaction mixture was acidified by HCl (6 N) to pH 4.0. The aqueous phase was extracted with ethyl acetate (3×20 mL) and the organic layer was combined, dried over Na₂SO₄, and concentrated. LC-MS: calculated [M+H]+ 366.20, found 366.46.

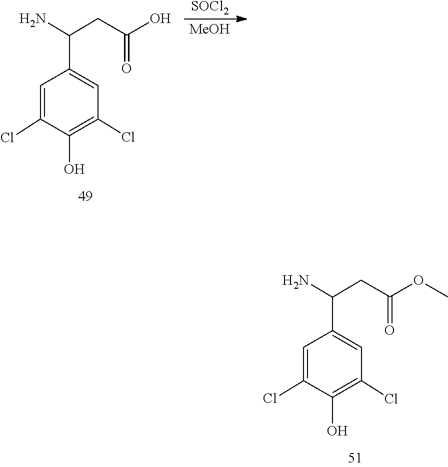

To a suspension of compound 49 (0.531 g, 2.12 mmol, 1 equiv.) in anhydrous methanol (10 mL) was added thionyl chloride (308 uL, 4.24 mmol, 2.0 equiv.) on ice bath. The reaction was warmed to room temperature and stirred overnight. The solvent was removed under reduced pressure and the product was directly used without further purification. LC-MS: calculated [M+H]+ 264.01, found 264.20.

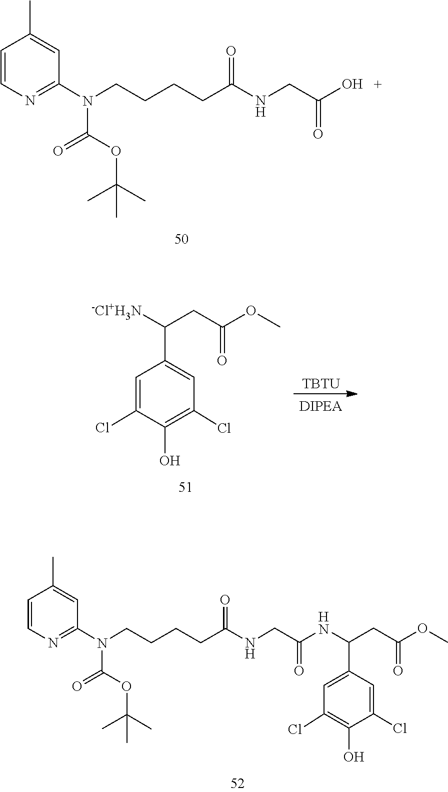

To a solution of compound 50 (150 mg, 0.410 mmol, 1 equiv.), compound 51 (148 mg, 0.492 mmol, 1.2 equiv.), and TBTU (158 mg, 0.492 mmol, 1.2 equiv.) in anhydrous DMF (5 mL) was added diisopropylethylamine (0.214 mL, 1.23 mmol, 3 equiv.) at 0° C. The reaction mixture was warmed to room temperature and stirred 3 hours. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 2-4% methanol in DCM.

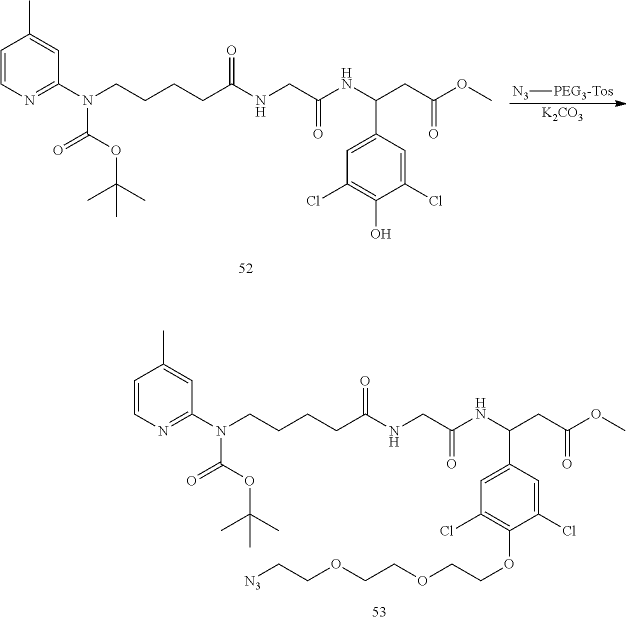

To a solution of compound 52 (80 mg, 0.130 mmol, 1 equiv.) and azido-PEG₃-OTs (86 mg, 0.262 mmol, 2 equiv.) in anhydrous DMF (2 mL) was added K₂CO₃ (36 mg, 0.262 mmol, 2 equiv.) at 0° C. The reaction mixture was stirred for 1 hr at 80° C. The solvent was removed by rotary evaporator. The product was purified by CombiFlash® using silica gel as the stationary phase and was eluted with 2-4% methanol in DCM. LC-MS: calculated [M+H]+768.28, found 769.

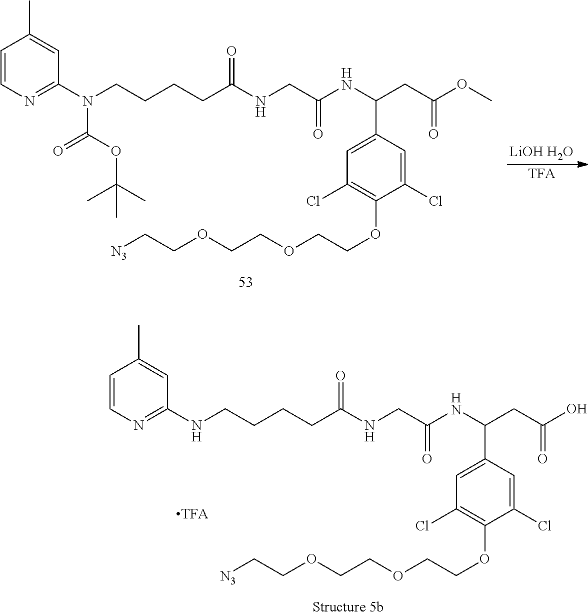 [SMILES: COC(=O)CC(NC(=O)CNC(=O)CCCCN(C(=O)OC(C)(C)C)c1cc(C)ccn1)c1cc(Cl)c(OCCOCCOCCN=[N+]=[N-])c(Cl)c1 ; "LiOH H2O" ; Cc1ccnc(NCCCCC(=O)NCC(=O)NC(CC(=O)O)c2cc(Cl)c(OCCOCCOCCN=[N+]=[N-])c(Cl)c2)c1 ; "TFA"]

To a solution of compound 53 (58 mg, 0.0755 mmol, 1.0 equiv.) in THE (2 mL) and water (2 mL) was added lithium hydroxide monohydrate (10 mg, 0.226 mmol, 3.0 equiv.) at room temperature. The mixture was stirred at room temperature for another 2 hours. The pH was adjusted to 3.0 by HCl (6N) and the aqueous phase was extracted with EtOAc (3×10 mL). The organic phase was combined, dried over Na₂SO₄, and concentrated. TFA (0.25 mL) and DCM (0.75 mL) was added into the residue and the mixture was stirred at room temperature for another 1 hour. The solvent was removed by rotary evaporator. LC-MS: calculated [M+H]+ 654.21, found 655.

### Structure 5.1b (3-(4-((14-azido-3,6,9,12-tetraoxatetradecyl)oxy)-3,5-dichlorophenyl)-3-(2-(5-((4-methylpyridin-2-yl)amino)pentanamido)acetamido)propanoic acid)

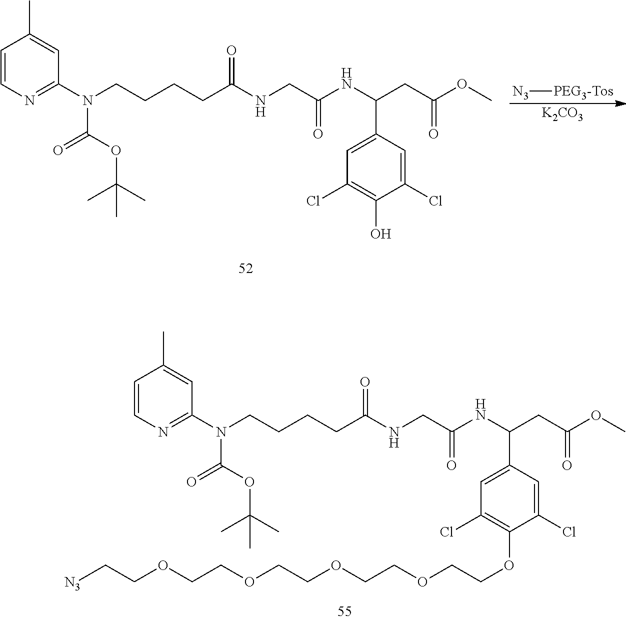

To a solution of compound 52 (100 mg, 0.163 mmol, 1 equiv.) and azido-PEG₅-OTs (205 mg, 0.491 mmol, 3 equiv.) in anhydrous DMF (2 mL) was added K₂CO₃ (68 mg, 0.491 mmol, 2 equiv.) at 0° C. The reaction mixture was stirred for 1 hour at 80° C. The solvent was removed by rotary evaporator. The product was purified by CombiFlash® using silica gel as the stationary phase and was eluted with 2-3% methanol in DCM. LC-MS: calculated [M+H]+ 856.33, found 857.07.

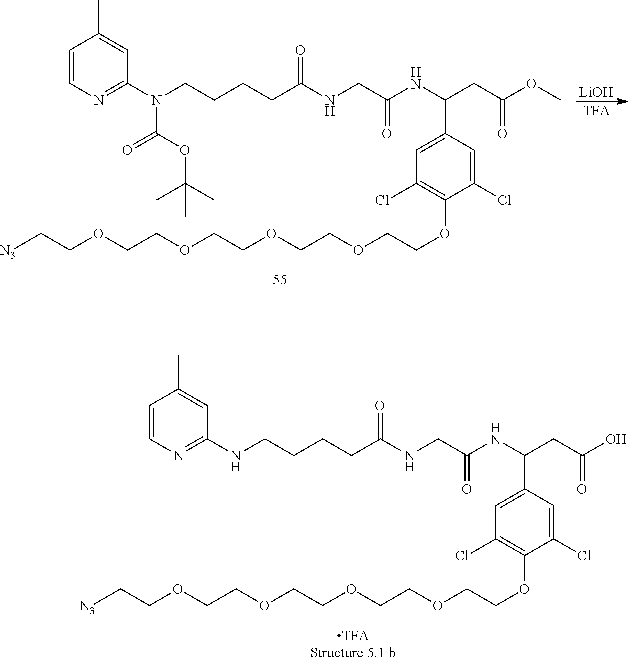

To a solution of compound 55 (119 mg, 0.139 mmol, 1.0 equiv.) in THE (4 mL) and water (4 mL) was added lithium hydroxide (10 mg, 0.417 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (2 mL) and DCM (2 mL) was added into the residue and the mixture was stirred at room temperature for another 3 hours. The solvent was removed by rotary evaporator. LC-MS: calculated [M+H]+ 742.27, found 743.02.

### Structure 5.2b (3-(4-((8-azidooctyl)oxy)-3,5-dichlorophenyl)-3-(2-(5-((4-methylpyridin-2-yl)amino)pentanamido)acetamido)propanoic acid)

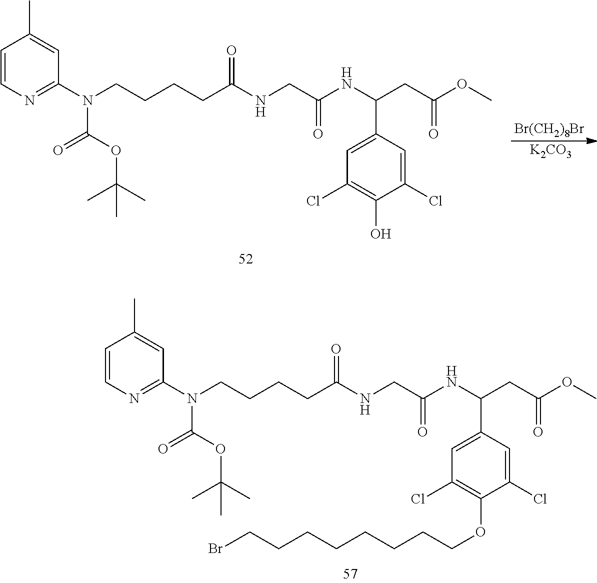

To a solution of compound 52 (89 mg, 0.14 mmol, 1 equiv.) and 1,8-dibromooctane (80 uL, 0.436 mmol, 3 equiv.) in acetone (2 mL) was added K₂CO₃ (60 mg, 0.436 mmol, 3 equiv.) at room temperature. The reaction mixture was stirred for 6 hours at 55° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 801.23, found 801.98.

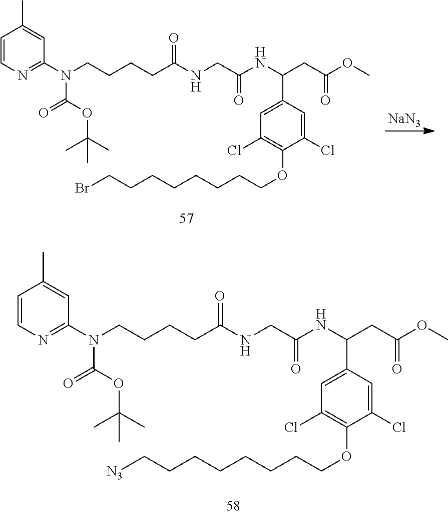 [SMILES: COC(=O)CC(NC(=O)CNC(=O)CCCCN(C(=O)OC(C)(C)C)c1cc(C)ccn1)c1cc(Cl)c(OCCCCCCCCBr)c(Cl)c1 ; COC(=O)CC(NC(=O)CNC(=O)CCCCN(C(=O)OC(C)(C)C)c1cc(C)ccn1)c1cc(Cl)c(OCCCCCCCCN=[N+]=[N-])c(Cl)c1 ; "NaN3"]

To a solution of compound 57 (97 mg, 0.114 mmol, 1 equiv.) in anhydrous DMF (2 mL) was added sodium azide (15 mg, 0.229 mmol, 2 equiv.) at room temperature. The reaction mixture was stirred for 2 hours at 80° C. The reaction was quenched by water and the aqueous layer was extracted with ethyl acetate (3×10 mL). The organic phase was combined, dried over Na₂SO₄, and concentrated. The product was used directly without further purification. LC-MS: calculated [M+H]+ 764.32, found 765.07.

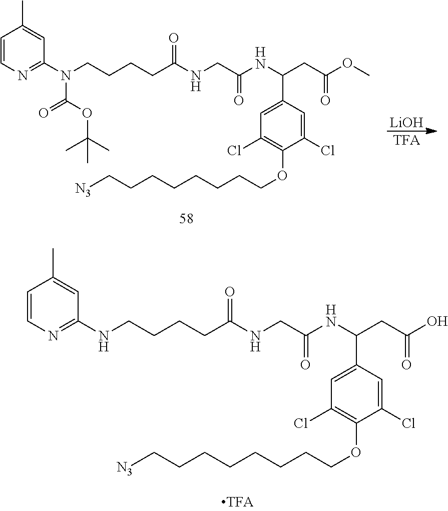 [SMILES: COC(=O)CC(NC(=O)CNC(=O)CCCCN(C(=O)OC(C)(C)C)c1cc(C)ccn1)c1cc(Cl)c(OCCCCCCCCN=[N+]=[N-])c(Cl)c1 ; "LiOH" ; Cc1ccnc(NCCCCC(=O)NCC(=O)NC(CC(=O)O)c2cc(Cl)c(OCCCCCCCCN=[N+]=[N-])c(Cl)c2)c1 ; "TFA"]

To a solution of compound 58 (78 mg, 0.101 mmol, 1.0 equiv.) in THE (2 mL) and water (2 mL) was added lithium hydroxide (7 mg, 0.304 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (2 mL) and DCM (2 mL) was added into the residue and the mixture was stirred at room temperature for another 3 hr. The solvent was removed by rotary evaporator. LC-MS: calculated [M+H]+ 650.25, found 650.83.

### Synthesis of Structure 6b, 6.1b, 6.2b, 6.3b, and 6.4b

### Structure 6b ((S)-3-(4-(4-(2-(2-(2-azidoethoxy)ethoxy)ethoxy)naphthalen-1-yl)phenyl)-3-(2-(4-((4-methylpyridin-2-yl)amino)butanamido)acetamido)propanoic acid)

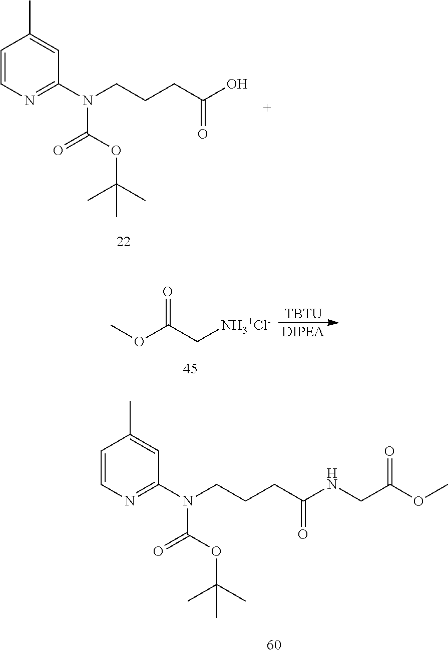

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+ 366.20, found 367.

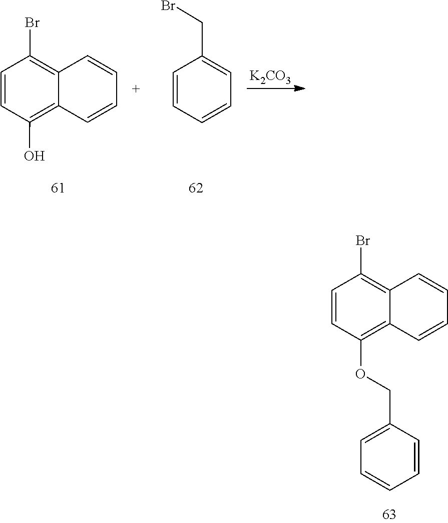

To a solution of compound 61 (2 g, 8.96 mmol, 1 equiv.), and compound 62 (2.13 mL, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

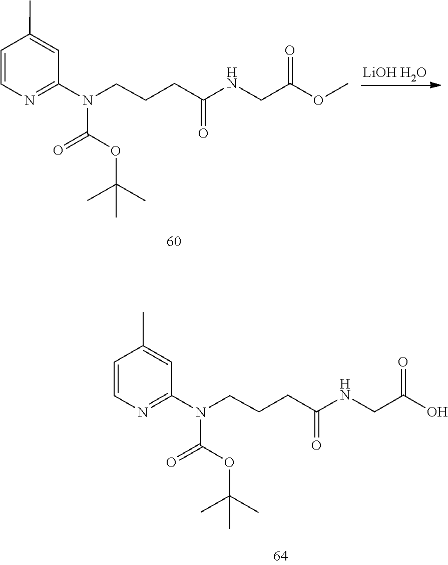

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+ 352.18, found 352.

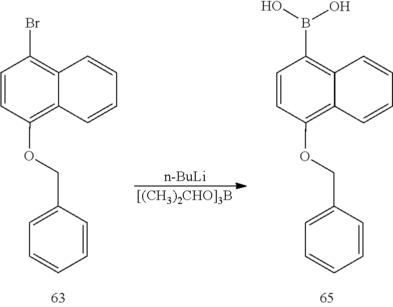

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH₄C₁ solution (20 mL) and the pH was adjusted to 3. The aqueous phase was extracted with EtOAc (3×20 mL) and the organic phase was combined, dried over Na₂SO₄, and concentrated.

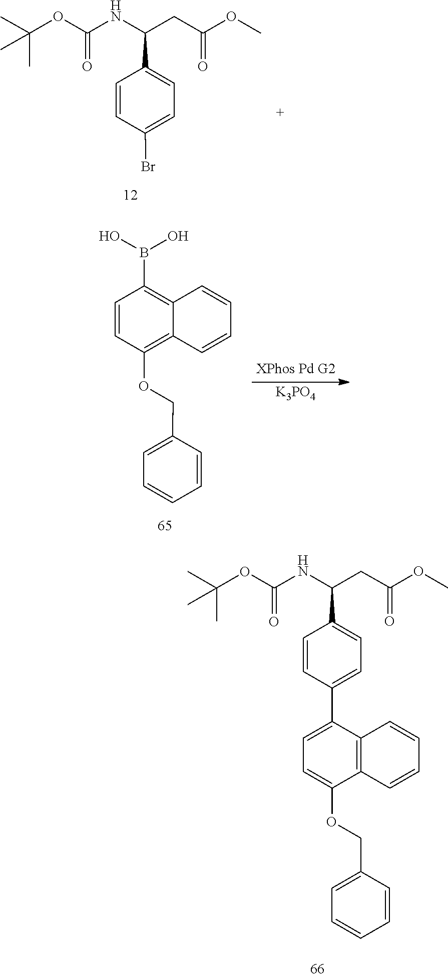

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THE (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+ 512.24, found 512.56.

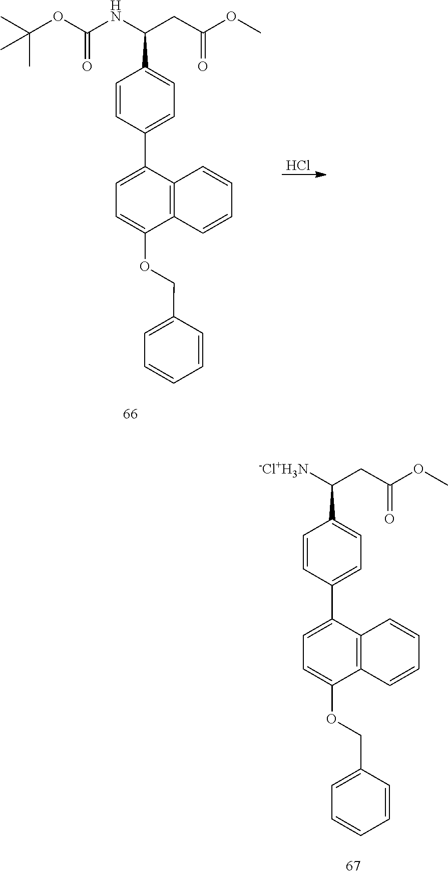

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 412.18, found 412.46.

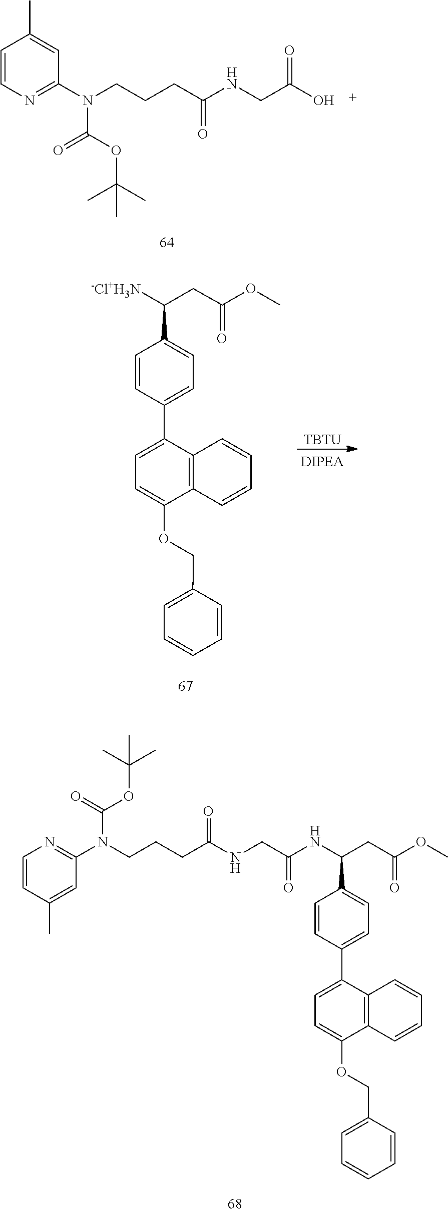

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+ 745.35, found 746.08.

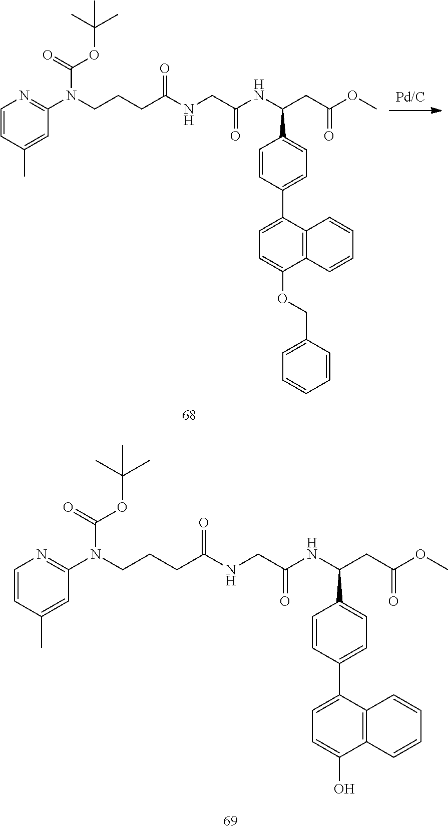 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 10% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+ 655.31, found 655.87.

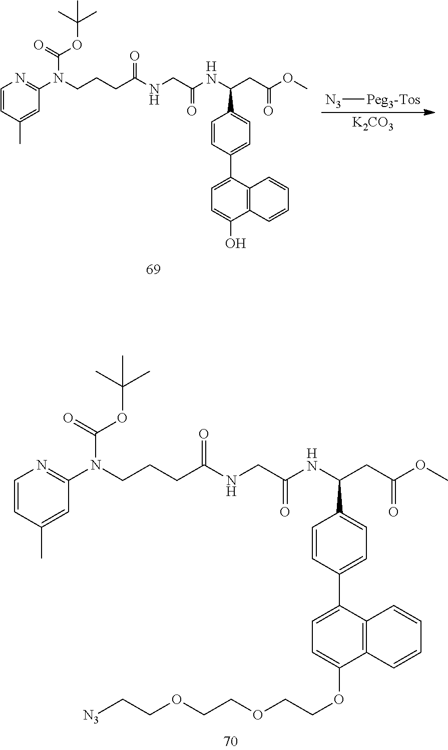

To a solution of compound 69 (100 mg, 0.152 mmol, 1 equiv.) and azido-PEG₃-OTs (100 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. The product was separated by CombiFlash® using silica gel as the stationary phase. LC-MS: calculated [M+H]+ 812.39, found 813.14.

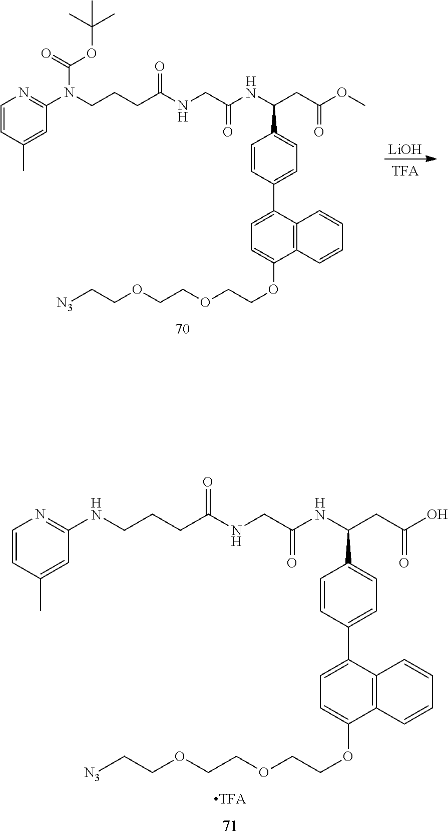 [SMILES: COC(=O)C[C@H](NC(=O)CNC(=O)CCCN(C(=O)OC(C)(C)C)c1cc(C)ccn1)c1ccc(-c2ccc(OCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCN=[N+]=[N-])c4ccccc34)cc2)c1]

To a solution of compound 70 (77 mg, 0.0948 mmol, 1.0 equiv.) in THE (2 mL) and water (2 mL) was added lithium hydroxide (7 mg, 0.284 mmol, 3.0 equiv.) at room temperature. The mixture was stirred at room temperature for another 2 hours. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hours. The solvent was removed by rotary evaporator. LC-MS: calculated [M+H]+ 698.32, found 698.81.

### Structure 6.1b ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

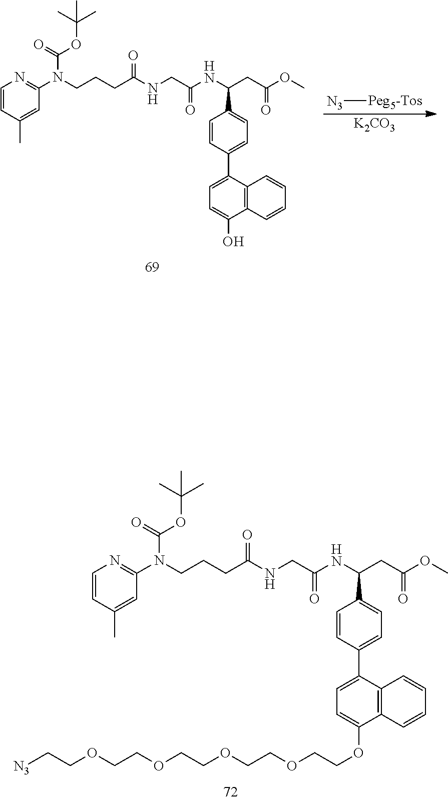

To a solution of compound 69 (100 mg, 0.152 mmol, 1 equiv.) and azido-PEG₅-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 900.40, found 901.46.

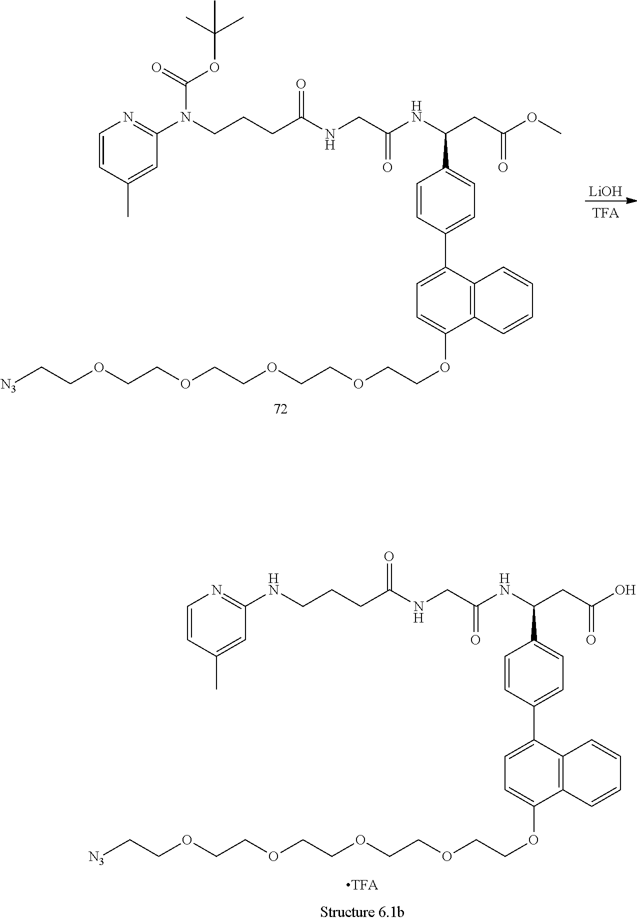 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 72 (59 mg, 0.0656 mmol, 1.0 equiv.) in THE (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 786.37, found 786.95.

### Structure 6.2b ((S)-3-(4-(4-((8-azidooctyl)oxy)naphthalen-1-yl)phenyl)-3-(2-(4-((4-methylpyridin-2-yl)amino)butanamido)acetamido)propanoic acid)

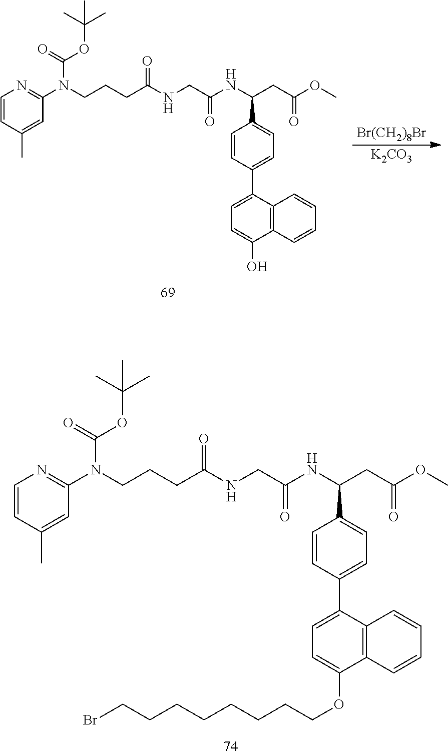

To a solution of compound 69 (150 mg, 0.229 mmol, 1 equiv.) and 1,8-dibromooctane (127 uL, 0.687 mmol, 3 equiv.) in acetone (2 mL) was added K₂CO₃ (95 mg, 0.687 mmol, 3 equiv.) at room temperature. The reaction mixture was stirred for overnight at 55° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 845.34, found 845.91.

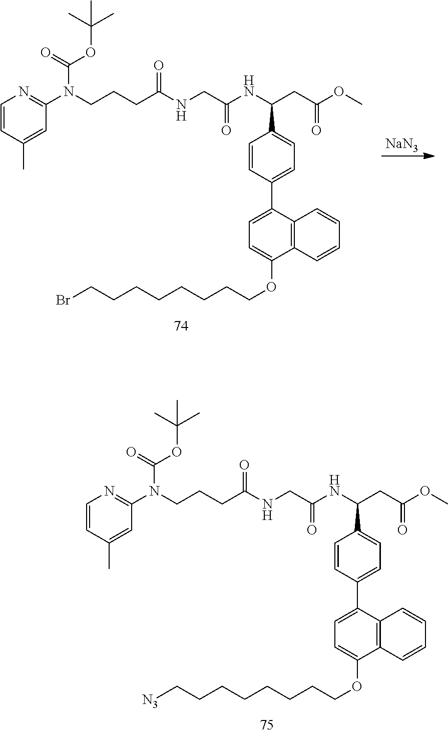 [SMILES: COC(=O)C[C@H](NC(=O)CNC(=O)CCCN(C(=O)OC(C)(C)C)c1cc(C)ccn1)c1ccc(-c2ccc(OCCCCCCCCBr)c3ccccc23)cc1 ; COC(=O)C[C@H](NC(=O)CNC(=O)CCCN(C(=O)OC(C)(C)C)c1cc(C)ccn1)c1ccc(-c2ccc(OCCCCCCCCN=[N+]=[N-])c3ccccc23)cc1 ; "NaN3"]

To a solution of compound 74 (97 mg, 0.114 mmol, 1 equiv.) in anhydrous DMF (2 mL) was added sodium azide (15 mg, 0.229 mmol, 2 equiv.) at room temperature. The reaction mixture was stirred for 2 hours at 80° C. The reaction was quenched by water and the aqueous layer was extracted with ethyl acetate (3×10 mL). The organic phase was combined, dried over Na₂SO₄, and concentrated. LC-MS: calculated [M+H]+ 808.43, found 809.00.

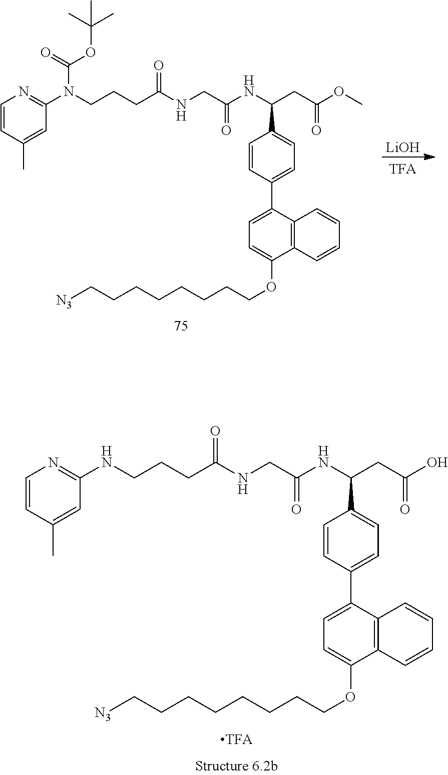 [SMILES: COC(=O)C[C@H](NC(=O)CNC(=O)CCCN(C(=O)OC(C)(C)C)c1cc(C)ccn1)c1ccc(-c2ccc(OCCCCCCCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCCCCCCCN=[N+]=[N-])c4ccccc34)cc2)c1]

To a solution of compound 75 (92 mg, 0.114 mmol, 1.0 equiv.) in THE (2 mL) and water (2 mL) was added lithium hydroxide (8 mg, 0.342 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 694.36, found 694.94.

### Structure 6.3b ((S)-3-(4-(4-((20-azido-3,6,9,12,15,18-hexaoxaicosyl)oxy)naphthalen-1-yl)phenyl)-3-(2-(4-((4-methylpyridin-2-yl)amino)butanamido)acetamido)propanoic acid)

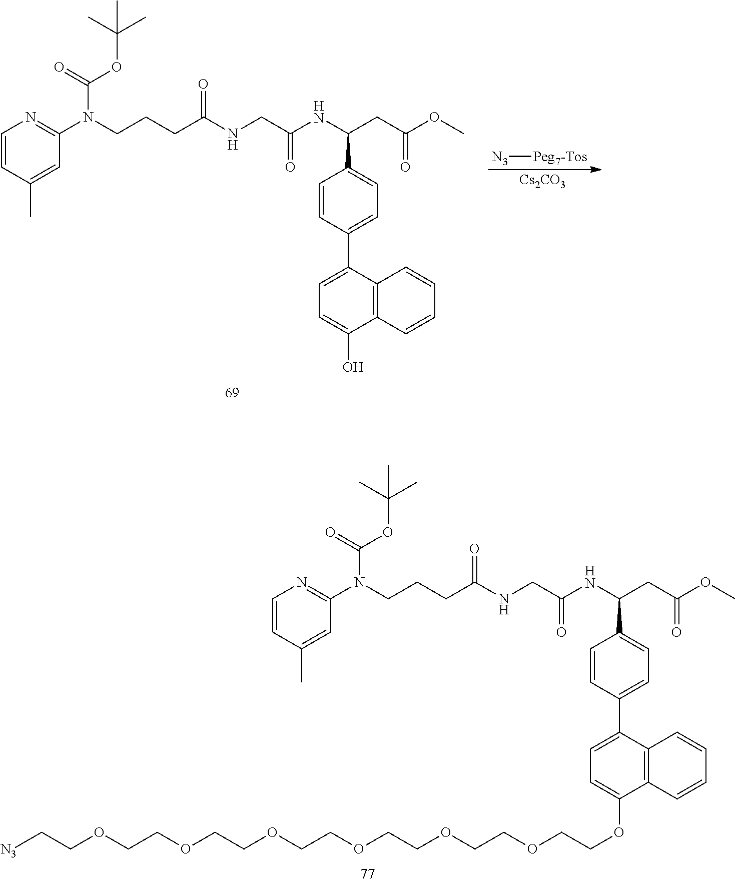

To a solution of compound 69 (100 mg, 0.152 mmol, 1 equiv.) and azido-PEG₇-OTs (154 mg, 0.305 mmol, 2 equiv.) in anhydrous DMF (2 mL) was added Cs₂CO₃ (100 mg, 0.305 mmol, 2 equiv.) at 0° C. The reaction mixture was stirred at 40° C. overnight. The reaction was quenched by saturated NaHCO₃ solution (10 mL) and the aqueous layer was extracted with ethyl acetate (3×10 mL). The organic phase was combined, dried over Na₂SO₄, and concentrated. The product was separated by CombiFlash® using silica gel as the stationary phase, and the product was eluted with 2-3% methanol in DCM. LC-MS: calculated [M+H]+ 988.50, found 989.14.

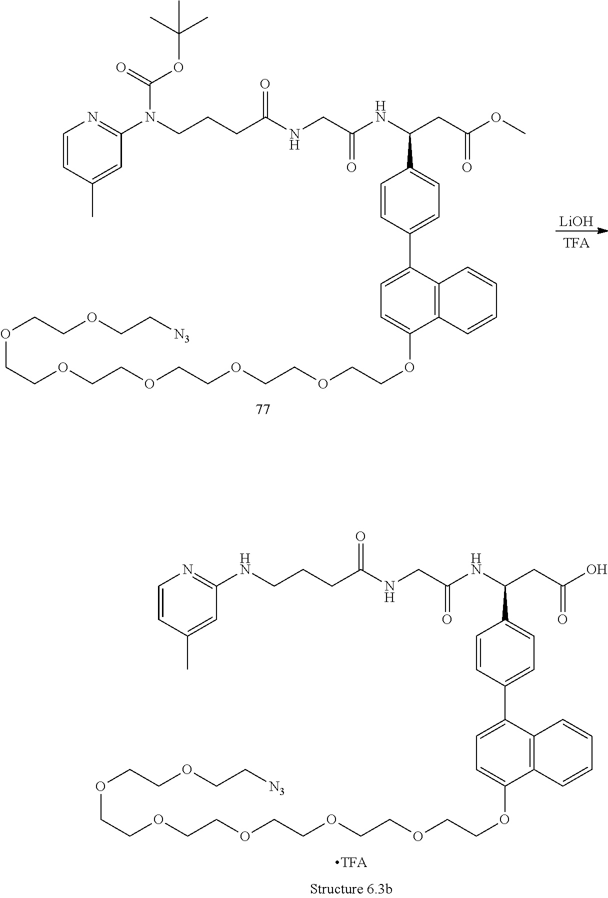 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCOCCOCCN=[N+]=[N-])c4ccccc34)cc2)c1]

To a solution of compound 21 (112 mg, 0.113 mmol, 1.0 equiv.) in THE (2 mL) and water (2 mL) was added lithium hydroxide (8 mg, 0.340 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hours. The solvent was removed by rotary evaporator. LC-MS: calculated [M+H]+ 874.43, found 875.08.

### Structure 6.4b ((S)-3-(4-(4-((35-azido-3,6,9,12,15,18,21,24,27,30,33-undecaoxapentatriacontyl)oxy)naphthalen-1-yl)phenyl)-3-(2-(4-((4-methylpyridin-2-yl)amino)butanamido)acetamido)propanoic acid)

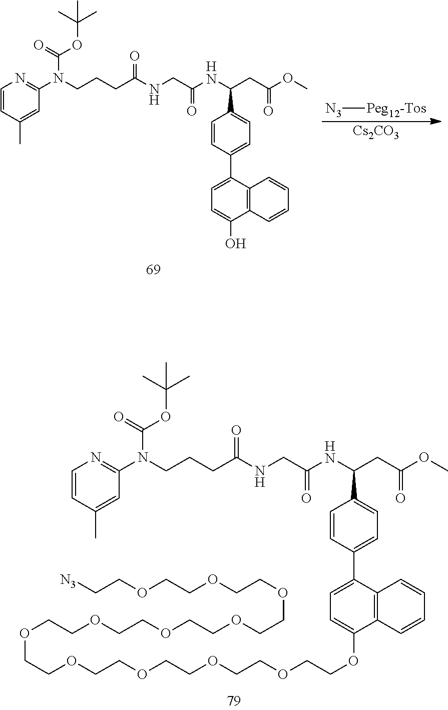

To a solution of compound 69 (80 mg, 0.122 mmol, 1 equiv.) and azido-PEG₁₂-OTs (184 mg, 0.244 mmol, 2 equiv.) in anhydrous DMF (2 mL) was added Cs₂CO₃ (80 mg, 0.244 mmol, 2 equiv.) at 0° C. The reaction mixture was stirred at 40° C. for 5 hours. The reaction was quenched by saturated NaHCO₃ solution (10 mL) and the aqueous layer was extracted with ethyl acetate (3×10 mL). The organic phase was combined, dried over Na₂SO₄, and concentrated. The product was separated by CombiFlash® using silica gel as the stationary phase and eluted with 2-3% methanol in DCM. LC-MS: calculated [M+H]+ 1208.63, found 1209.21.

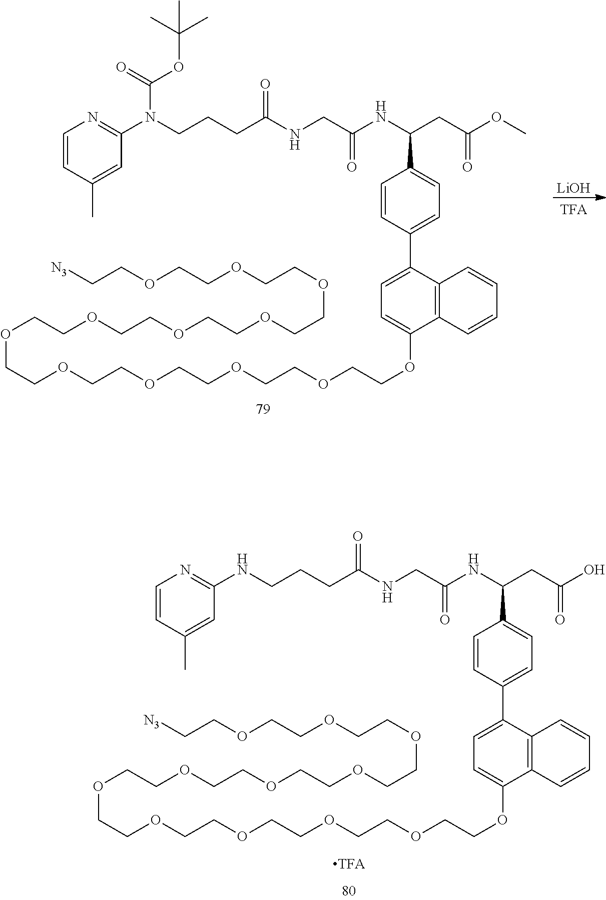 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCOCCOCCOCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCOCCOCCOCCOCCOCCOCCOCCN=[N+]=[N-])c4ccccc34)cc2)c1]

To a solution of compound 82 (100 mg, 0.0972 mmol, 1.0 equiv.) in THE (2 mL) and water (2 mL) was added lithium hydroxide (7 mg, 0.292 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 1094.56, 1095.05.

### Synthesis of Structure 7b ((R)-3-(4-(4-(2-(2-(2-azidoethoxy)ethoxy)ethoxy)naphthalen-1-yl)phenyl)-3-(2-(4-((4-methylpyridin-2-yl)amino)butanamido)acetamido)propanoic acid)

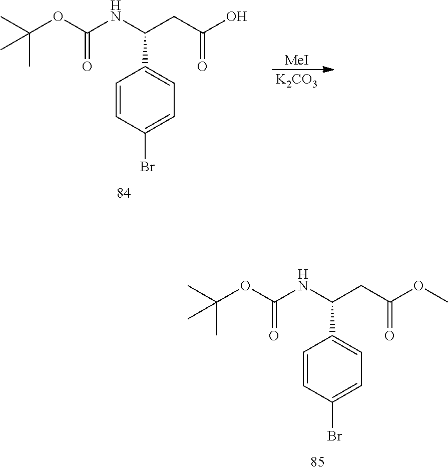

To a solution of compound 84 (1.0 g, 2.90 mmol, 1 equiv.) and potassium carbonate (0.60 g, 4.36 mmol, 1.5 equiv.) in anhydrous DMF (10 mL) was added methyl iodide (362 uL, 5.81 mmol, 2.0 equiv.) at room temperature. The reaction mixture was stirred at room temperature 1 hr. LC-MS: calculated [M+H]+ 358.06, found 358.34.

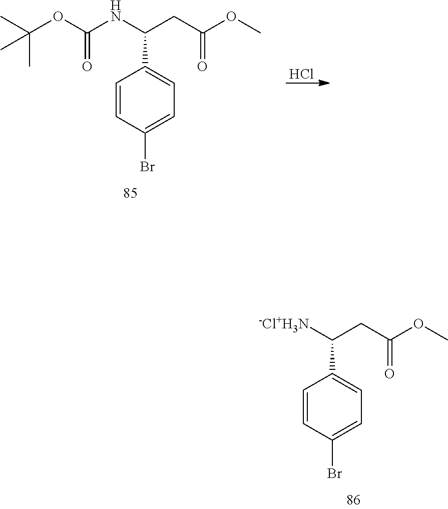

Compound 85 (1.0 g, 2.791 mmol, 1.0 equiv.) was cooled by ice bath. HCl in dioxane (7.0 mL, 27.91 mmol, 10 equiv.) was added into the flask. The reaction was warmed to room temperature and stirred for another 1 hour. The solvent was removed by rotary evaporator and the product was directly used without further purification. LC-MS: calculated [M+H]+ 258.01, found 257.97.

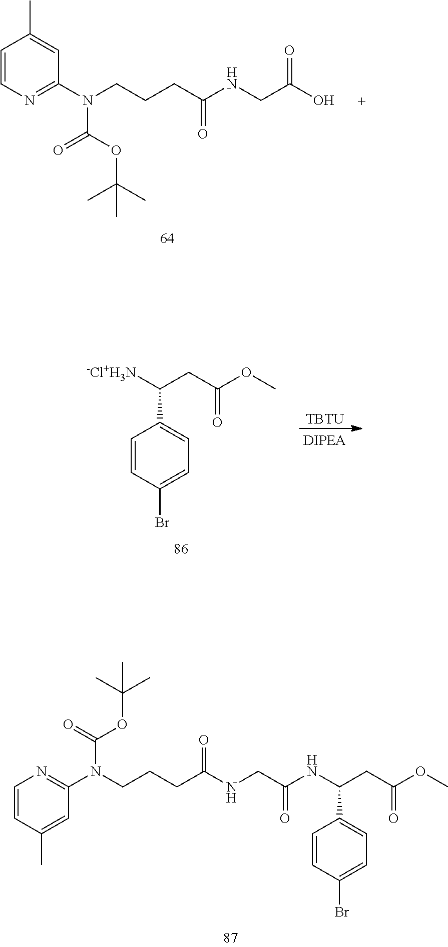

To a solution of compound 64 (790 mg, 2.248 mmol, 1 equiv.), compound 86 (728 mg, 2.473 mmol, 1.10 equiv.), and TBTU (866 mg, 2.698 mmol, 1.20 equiv.) in anhydrous DMF (15 mL) was added diisopropylethylamine (1.175 mL, 6.744 mmol, 3 equiv.) at 0° C.

The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 591.17, found 591.49.

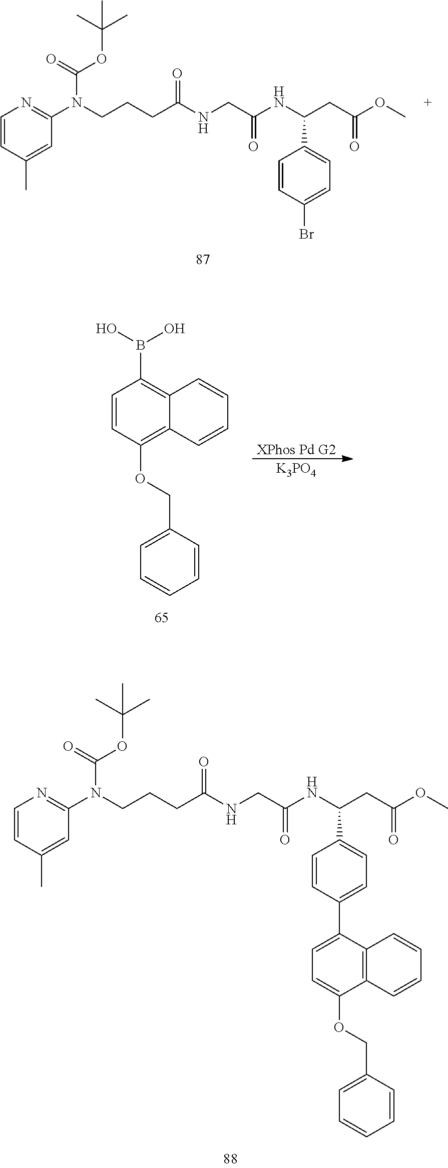

Compound 87 (200 mg, 0.338 mmol, 1.0 equiv.), compound 65 (141 mg, 0.507 mmol, 1.5 equiv.), XPhos Pd G2 (5.3 mg, 0.068 mmol, 0.02 equiv.), and K₃PO₄ (143 mg, 0.676 mmol, 2.0 equiv.) were mixed in a round-bottom flask. The flask was sealed with a screw-cap septum, and then evacuated and backfilled with nitrogen (this process was repeated a total of 3 times). Then, THE (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄ and concentrated. LC-MS: calculated [M+H]+ 745.35, found 746.08.

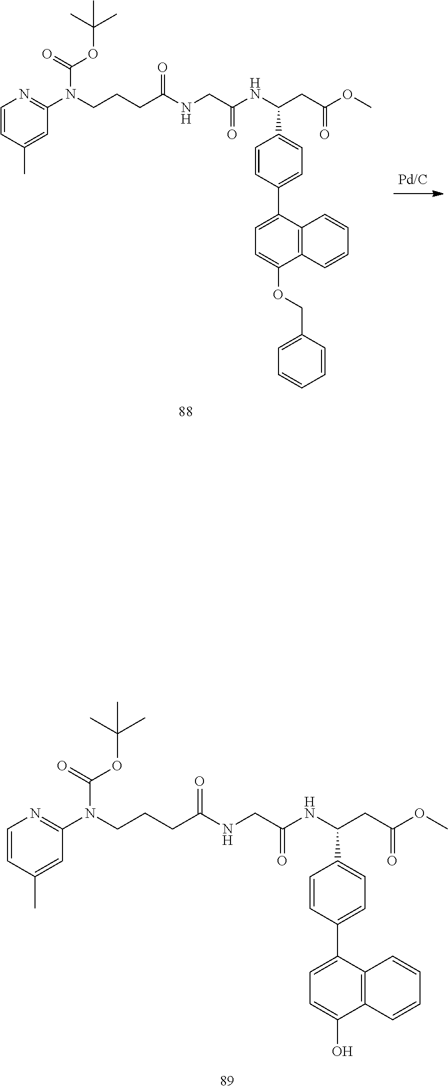 [SMILES: COC(=O)C[C@@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@@H](NC(=O)CNC(=O)CCCN(C(=O)OC(C)(C)C)c1cc(C)ccn1)c1ccc(-c2ccc(O)c3ccccc23)cc1]

To a solution of compound 88 (0.247 g, 0.331 mmol, 1 equiv.) in ethyl acetate (10 mL) was added 10% Pd/C (100 mg) at room temperature. The reaction mixture was stirred at room temperature for overnight. The catalyst was removed by filtration through Celite® and the product was used directly without further purification. LC-MS: calculated [M+H]+ 655.31, found 655.96.

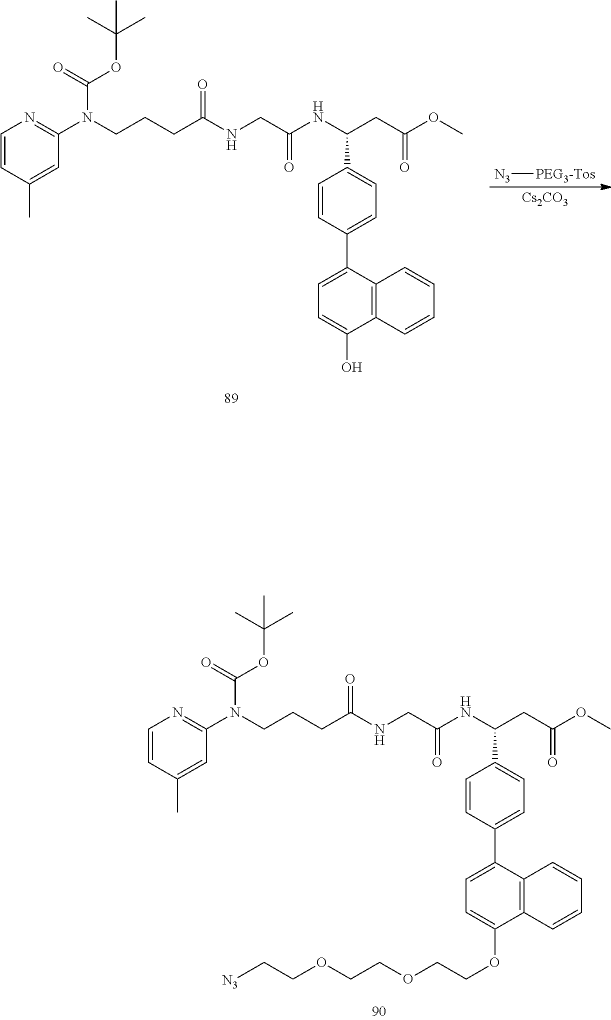

To a solution of compound 89 (50 mg, 0.076 mmol, 1 equiv.) and azido-PEG₃-OTs (50 mg, 0.152 mmol, 2 equiv.) in anhydrous DMF (2 mL) was added Cs₂CO₃ (50 mg, 0.152 mmol, 2 equiv.) at 0° C. The reaction mixture was stirred for 72 hr at room temperature. The reaction was quenched by saturated NaHCO₃ solution (10 mL) and the aqueous layer was extracted with ethyl acetate (3×10 mL). The organic phase was combined, dried over Na₂SO₄, and concentrated. The product was separated by CombiFlash® using silica gel as the stationary phase and was eluted with 4% MeOH in DCM. LC-MS: calculated [M+H]+ 812.39, found 813.14.

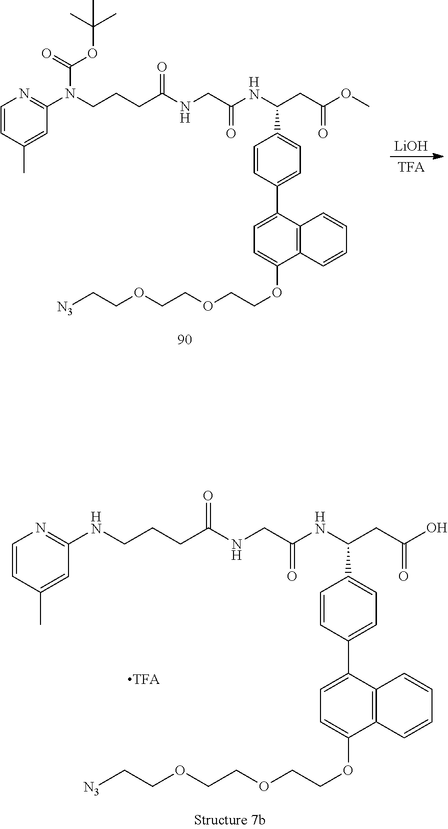 [SMILES: COC(=O)C[C@@H](NC(=O)CNC(=O)CCCN(C(=O)OC(C)(C)C)c1cc(C)ccn1)c1ccc(-c2ccc(OCCOCCOCCN=[N+]=[N-])c3ccccc23)cc1 ; "LiOH" ; Cc1ccnc(NCCCC(=O)NCC(=O)N[C@H](CC(=O)O)c2ccc(-c3ccc(OCCOCCOCCN=[N+]=[N-])c4ccccc34)cc2)c1 ; "TFA"]

To a solution of compound 90 (36 mg, 0.0443 mmol, 1.0 equiv.) in THE (2 mL) and water (2 mL) was added lithium hydroxide (3 mg, 0.133 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 698.32, found 698.90.

### Synthesis of Structure 8b ((S)-3-(4-(7-(2-(2-(2-azidoethoxy)ethoxy)ethoxy)naphthalen-1-yl)phenyl)-3-(2-(4-((4-methylpyridin-2-yl)amino)butanamido)acetamido)propanoic acid)

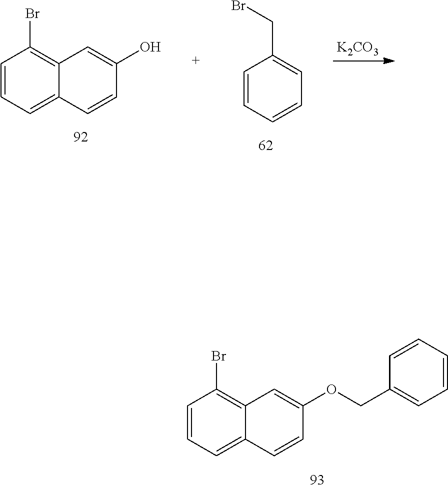

To a solution of compound 92 (1.0 g, 4.48 mmol, 1 equiv.), and compound 62 (1.06 mL, 8.96 mmol, 2 equiv.) in anhydrous DMF (10 mL) was added K₂CO₃ (1.24 g, 8.96 mmol, 2 equiv.) at room temperature. The reaction mixture was stirred at 80° C. overnight. The reaction was quenched by saturated NaHCO₃ solution (10 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 5% ethyl acetate in hexane.

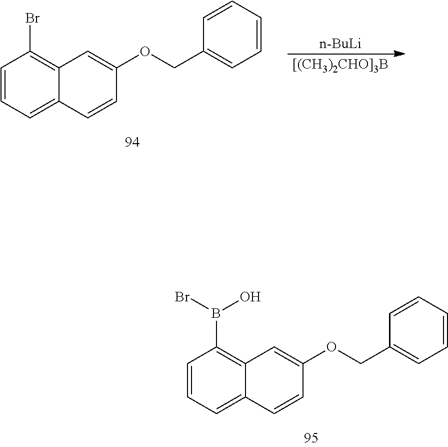

To a solution of compound 94 (0.5 g, 1.596 mmol, 1.0 equiv.) in anhydrous THF (10 mL) was added n-BuLi in hexane (0.96 mL, 2.394 mmol, 1.5 equiv.) drop-wise at −78° C. The reaction was kept at −78° C. for another 1 hour. Triisopropylborate (0.553 mL, 2.394 mmol, 1.5 equiv.) was then added into the mixture at −78° C. The reaction was then warmed up to room temperature and stirred for another 1 hour. The reaction was quenched by saturated NH₄Cl solution (20 mL) and the pH was adjusted to 3. The aqueous phase was extracted with EtOAc (3×20 mL) and the organic phase was combined, dried over Na₂SO₄, and concentrated. The solid was triturated with hexane and filtered. The product was used directly without further purification. LC-MS: calculated [M-H]− 277.11, found 277.35.

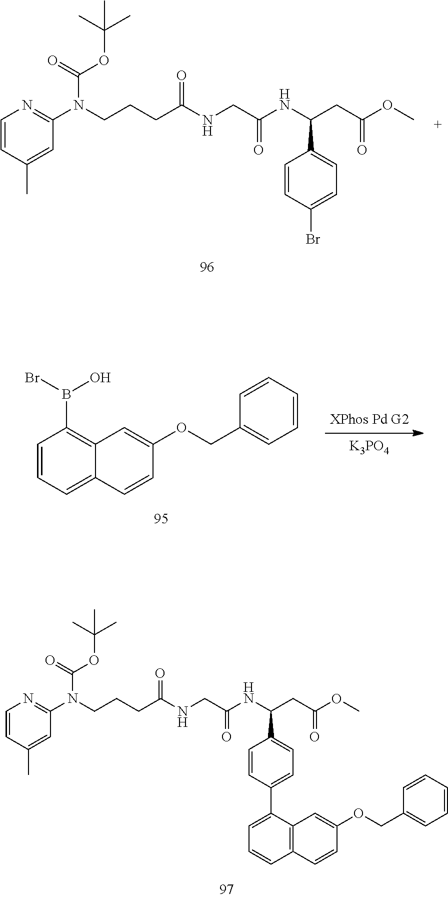

Compound 96 (100 mg, 0.169 mmol, 1.0 equiv.), compound 95 (70 mg, 0.253 mmol, 1.5 equiv.), XPhos Pd G2 (2.7 mg, 0.0034 mmol, 0.02 equiv.), and K₃PO₄ (72 mg, 0.338 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 3% methanol in DCM.

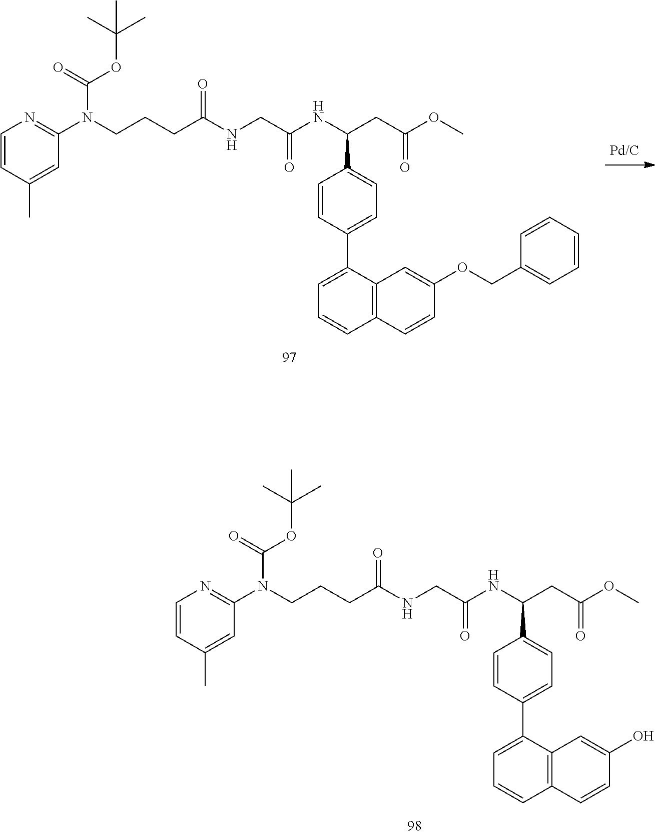 [SMILES: COC(=O)C[C@H](NC(=O)CNC(=O)CCCN(C(=O)OC(C)(C)C)c1cc(C)ccn1)c1ccc(-c2cccc3ccc(OCc4ccccc4)cc23)cc1 ; COC(=O)C[C@H](NC(=O)CNC(=O)CCCN(C(=O)OC(C)(C)C)c1cc(C)ccn1)c1ccc(-c2cccc3ccc(O)cc23)cc1]

To a solution of compound 97 (0.116 g, 0.157 mmol, 1 equiv.) in ethyl acetate (10 mL) was added 10% Pd/C (100 mg) at room temperature. The reaction mixture was stirred at room temperature for overnight. The catalyst was removed by filtration through Celite® and the product was used directly without further purification. LC-MS: calculated [M+H]+ 655.31, found 655.87.

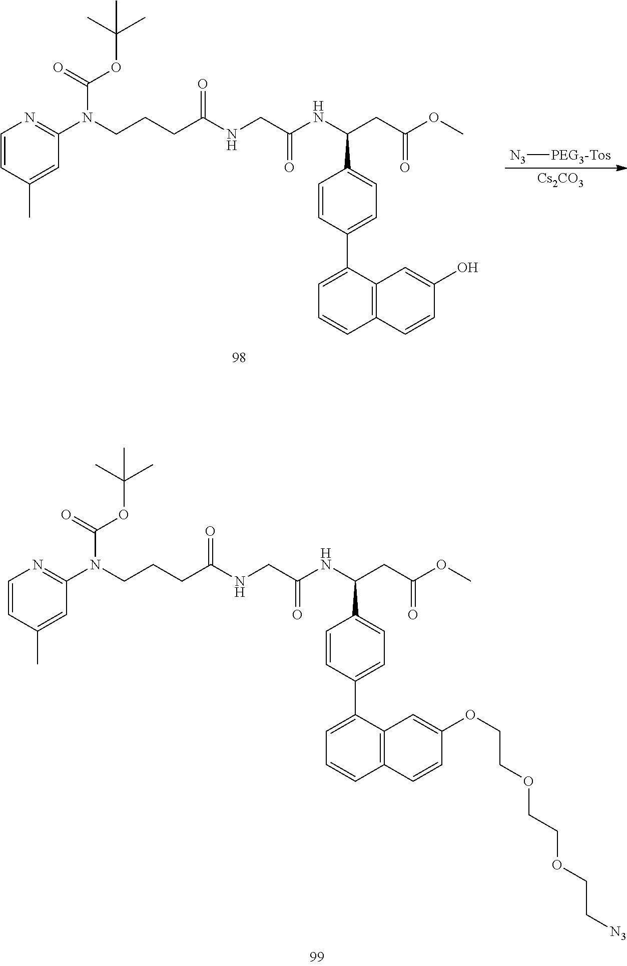

To a solution of compound 98 (87 mg, 0.133 mmol, 1 equiv.) and azido-PEG₃-OTs (87 mg, 0.266 mmol, 2 equiv.) in anhydrous DMF (2 mL) was added Cs₂CO₃ (87 mg, 0.266 mmol, 2 equiv.) at room temperature. The reaction mixture was stirred at 40° C. for 6 hours. The reaction was quenched by saturated NaHCO₃ solution (10 mL) and the aqueous layer was extracted with ethyl acetate (3×10 mL). The organic phase was combined, dried over Na₂SO₄, and concentrated. The product was separated by CombiFlash® using silica gel as the stationary phase and was eluted with 3-4% MeOH in DCM. LC-MS: calculated [M+H]+ 812.39, found 813.05.

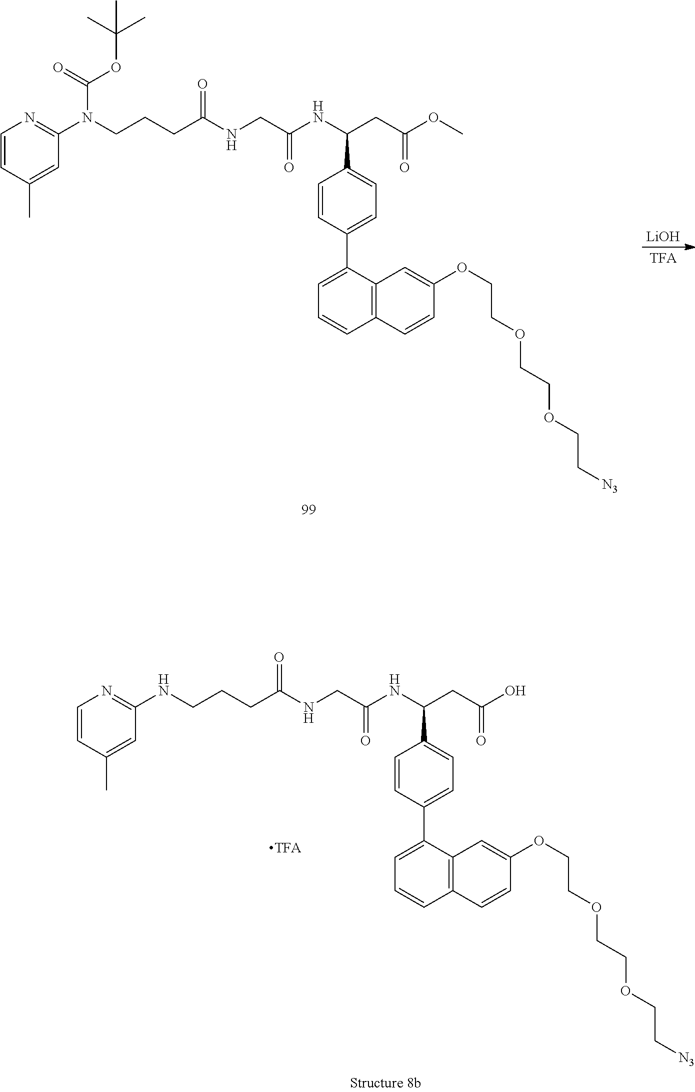 [SMILES: COC(=O)C[C@H](NC(=O)CNC(=O)CCCN(C(=O)OC(C)(C)C)c1cc(C)ccn1)c1ccc(-c2cccc3ccc(OCCOCCOCCN=[N+]=[N-])cc23)cc1 ; "LiOH" ; Cc1ccnc(NCCCC(=O)NCC(=O)N[C@@H](CC(=O)O)c2ccc(-c3cccc4ccc(OCCOCCOCCN=[N+]=[N-])cc34)cc2)c1 ; "TFA"]

To a solution of compound 99 (65 mg, 0.0801 mmol, 1.0 equiv.) in THE (2 mL) and water (2 mL) was added lithium hydroxide (6 mg, 0.240 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 698.32, found 698.99.

### Synthesis of Structure 9b ((14S,17R)-1-azido-14-(4-((4-methylpyridin-2-yl)amino)butanamido)-17-(4-(naphthalen-1-yl)phenyl)-15-oxo-3,6,9,12-tetraoxa-16-azanonadecan-19-oic acid)

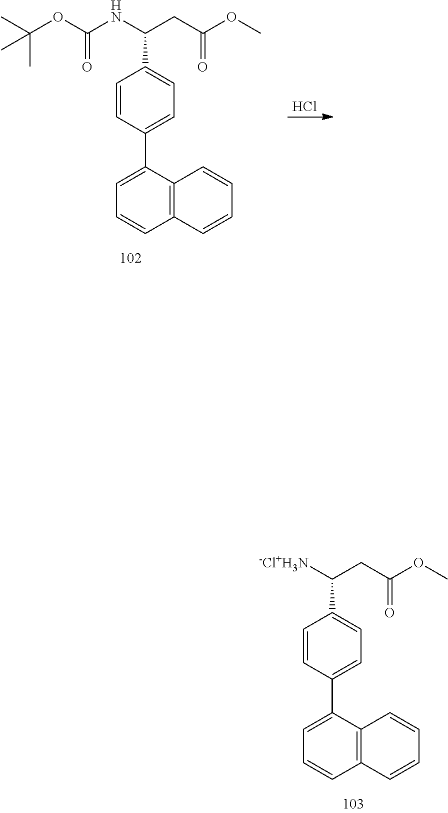

Compound 102 (0.19 g, 0.468 mmol, 1.0 equiv.) was cooled by ice bath. HCl in dioxane (2.35 mL, 9.37 mmol, 20 equiv.) was added into the flask. The reaction was warmed to room temperature and stirred for another 1 hr. The solvent was removed by rotary evaporator and the product was directly used without further purification. LC-MS: calculated [M+H]+ 306.14, found 306.51.

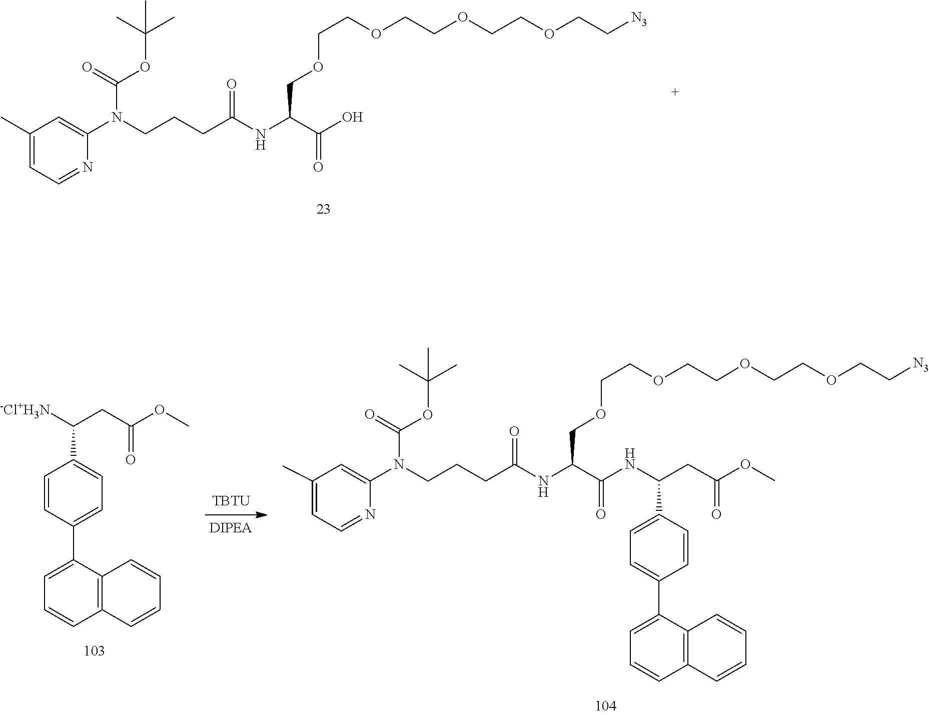

To a solution of compound 23 (110 mg, 0.188 mmol, 1 equiv.), compound 103 (71 mg, 0.207 mmol, 1.10 equiv.), and TBTU (72.7 mg, 0.226 mmol, 1.20 equiv.) in anhydrous DMF (2 mL) was added diisopropylethylamine (0.1 mL, 0.566 mmol, 3 equiv.) at 0° C. The reaction mixture was warmed to room temperature and stirred for another 1 hour. The reaction was quenched by saturated NaHCO₃ aqueous solution (10 mL) and the product was extracted with ethyl acetate (3×10 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 870.43, found 871.12.

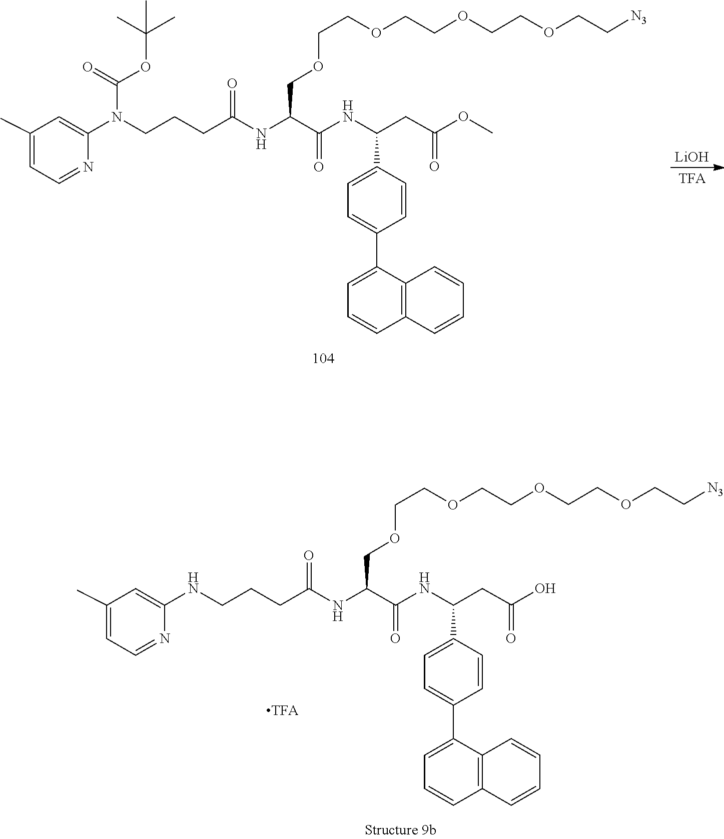 [SMILES: COC(=O)C[C@@H](NC(=O)[C@H](COCCOCCOCCOCCN=[N+]=[N-])NC(=O)CCCN(C(=O)OC(C)(C)C)c1cc(C)ccn1)c1ccc(-c2cccc3ccccc23)cc1 ; "LiOH" ; Cc1ccnc(NCCCC(=O)N[C@@H](COCCOCCOCCOCCN=[N+]=[N-])C(=O)N[C@H](CC(=O)O)c2ccc(-c3cccc4ccccc34)cc2)c1 ; "TFA"]

To a solution of compound 104 (110 mg, 0.126 mmol, 1.0 equiv.) in THE (2 mL) and water (2 mL) was added lithium hydroxide (9 mg, 0.379 mmol, 3.0 equiv.) at room temperature. The mixture was stirred at room temperature for another 1 hour. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hours. The solvent was removed by rotary evaporator. LC-MS: calculated [M+H]+ 756.36, found 756.88.

### Synthesis of Structure 10b ((S)-3-(4-(5-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

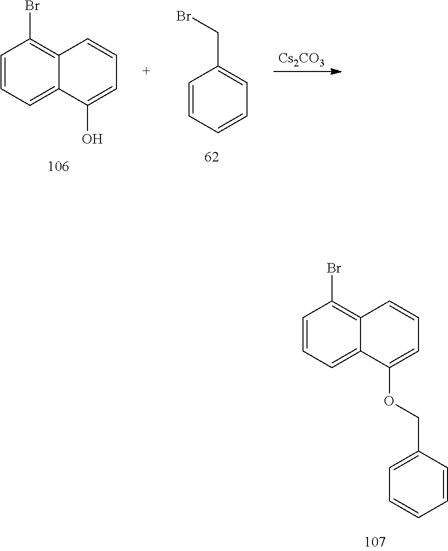

To a solution of compound 106 (1.0 g, 4.48 mmol, 1 equiv.), and compound 62 (1.06 mL, 8.96 mmol, 2 equiv.) in anhydrous DMF (10 mL) was added Cs₂CO₃ (2.92 g, 8.96 mmol, 2 equiv.) at room temperature. The reaction mixture was stirred at room temperature overnight. The reaction was quenched by water solution (20 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 5% ethyl acetate in hexane.

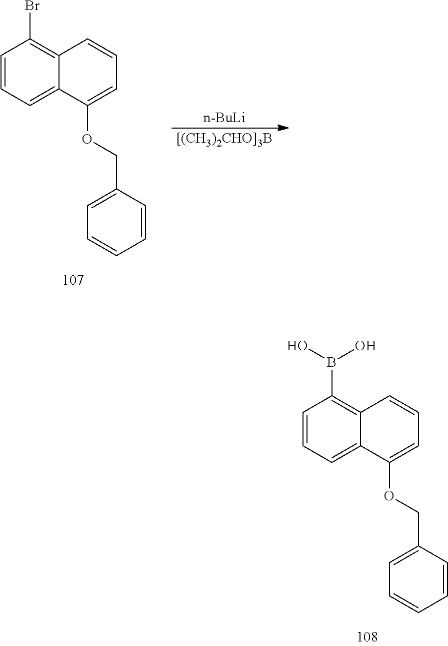

To a solution of compound 107 (1.188 g, 3.793 mmol, 1.0 equiv.) in anhydrous THE (10 mL) was added n-BuLi in hexane (2.27 mL, 5.689 mmol, 1.5 equiv.) drop-wise at 78° C. The reaction was kept at −78° C. for another 1 hour. Triisopropylborate (1.31 mL, 5.689 mmol, 1.5 equiv.) was then added into the mixture at −78° C. The reaction was then warmed up to room temperature and stirred for another 1 hour. The reaction was quenched by saturated NH₄Cl solution (20 mL) and the pH was adjusted to 3. The aqueous phase was extracted with EtOAc (3×20 mL) and the organic phase was combined, dried over Na₂SO₄, and concentrated. The solid was triturated with hexane and filtered. The product was used directly without further purification. LC-MS: calculated [M-H]−, 277.11, found 277.26.

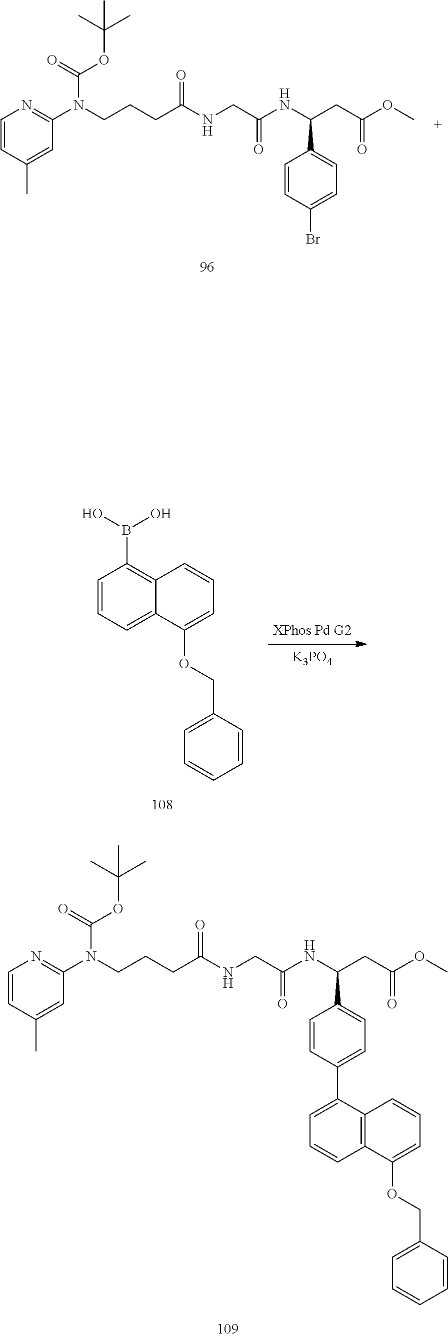

Compound 96 (100 mg, 0.169 mmol, 1.0 equiv.), compound 108 (70 mg, 0.253 mmol, 1.5 equiv.), XPhos Pd G2 (2.7 mg, 0.0034 mmol, 0.02 equiv.), and K₃PO₄ (72 mg, 0.338 mmol, 2.0 equiv.) were mixed in a round-bottom flask. The flask was sealed with a screw-cap septum, and then evacuated and backfilled with nitrogen (this process was repeated a total of 3 times). Then, THE (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 3% methanol in DCM. LC-MS: calculated [M+H]+ 745.35, found 745.99.

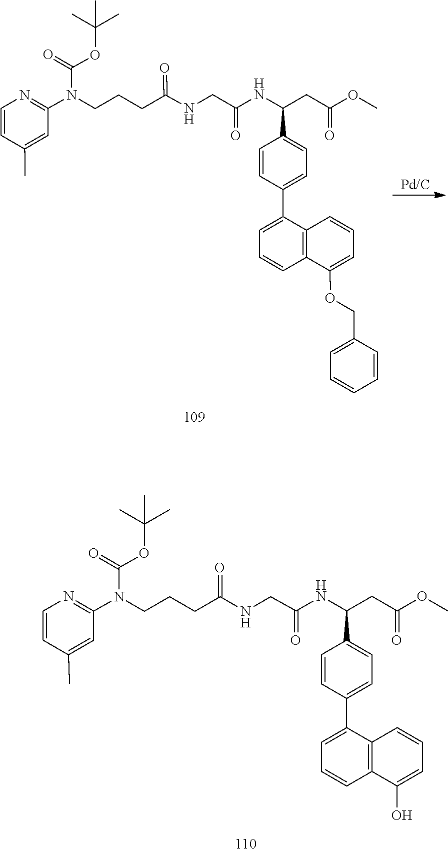 [SMILES: COC(=O)C[C@H](NC(=O)CNC(=O)CCCN(C(=O)OC(C)(C)C)c1cc(C)ccn1)c1ccc(-c2cccc3c(OCc4ccccc4)cccc23)cc1 ; COC(=O)C[C@H](NC(=O)CNC(=O)CCCN(C(=O)OC(C)(C)C)c1cc(C)ccn1)c1ccc(-c2cccc3c(O)cccc23)cc1]

To a solution of compound 109 (0.135 g, 0.181 mmol, 1 equiv.) in ethyl acetate (10 mL) was added 10% Pd/C (100 mg) at room temperature. The reaction mixture was stirred at room temperature for overnight. The catalyst was removed by filtration through Celite® and the product was used directly without further purification. LC-MS: calculated [M+H]+ 655.31, found 655.87.

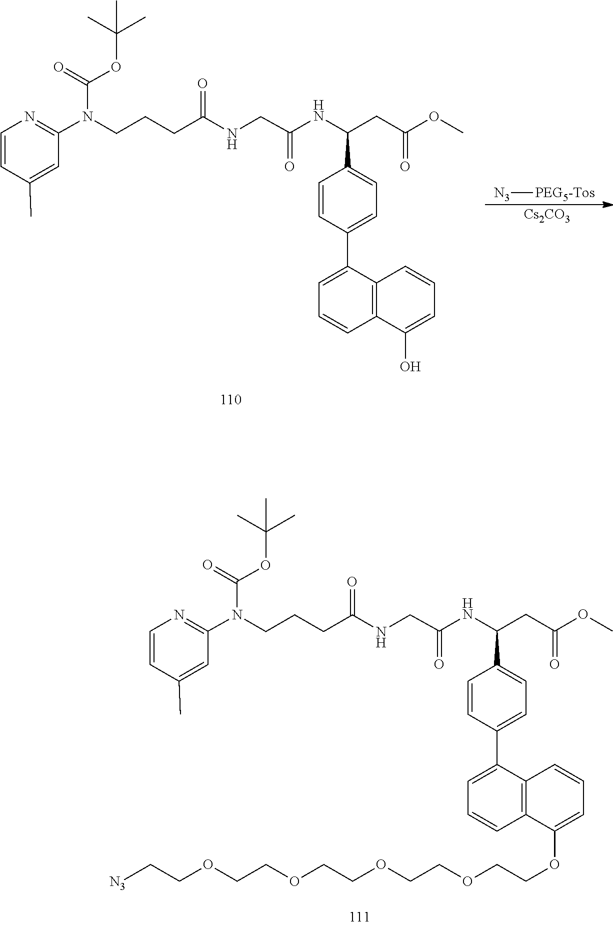

To a solution of compound 110 (50 mg, 0.0764 mmol, 1 equiv.) and azido-PEG5-OTs (64 mg, 0.152 mmol, 2 equiv.) in anhydrous DMF (2 mL) was added Cs₂CO₃ (50 mg, 0.152 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4% methanol in DCM. The yield is 62%. LC-MS: calculated [M+H]+ 900.44, found 901.19.

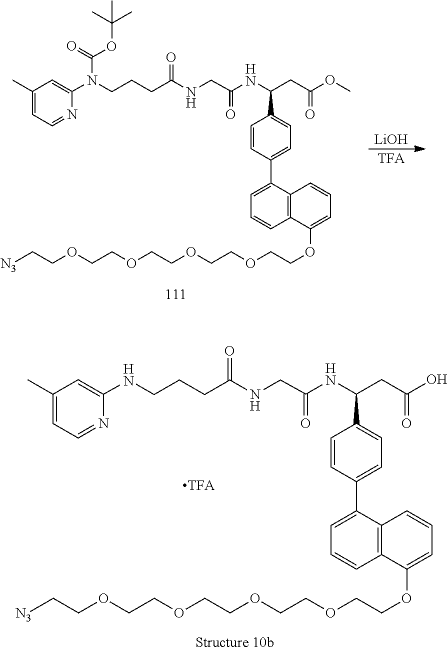

To a solution of compound 111 (43 mg, 0.0478 mmol, 1.0 equiv.) in THE (2 mL) and water (2 mL) was added lithium hydroxide (3.4 mg, 0.143 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 786.37, found 787.04.

### Synthesis of Structure 11b ((S)-3-(4-(4-((14-azido-3,6,9,12-tetraoxatetradecyl)oxy)naphthalen-1-yl)phenyl)-3-((S)-1-(4-((4-methylpyridin-2-yl)amino)butanoyl)pyrrolidine-2-carboxamido)propanoic acid)

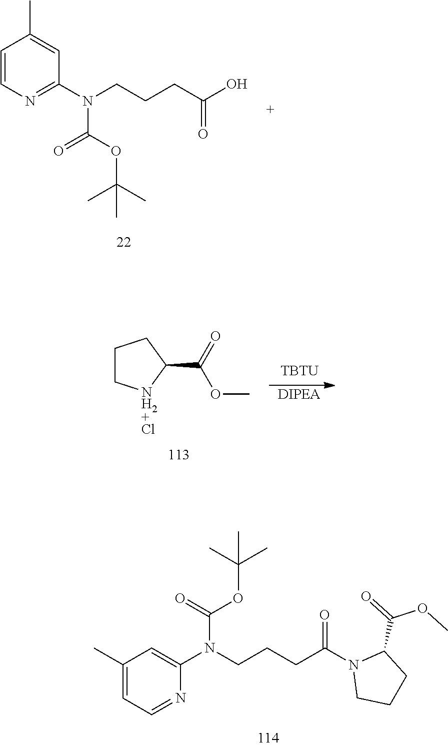

To a solution of compound 22 (500 mg, 1.698 mmol, 1 equiv.), compound 113 (295 mg, 1.783 mmol, 1.05 equiv.), and TBTU (654 mg, 2.038 mmol, 1.2 equiv.) in anhydrous DMF (10 mL) was added diisopropylethylamine (0.888 mL, 5.096 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×10 mL). The organic phase was combined, dried over Na₂SO₄, and concentrated. The product was purified by CombiFlash® using silica gel as the stationary phase and was eluted with 2-3% methanol in DCM. The yield is 98.72%. LC-MS: calculated [M+H]+ 406.23, found 406.07.

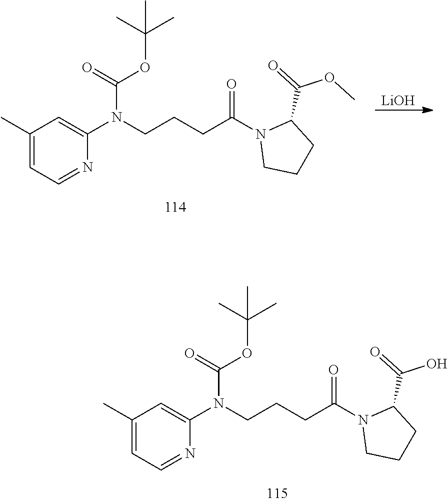

To a solution of compound 114 (0.68 g, 1.676 mmol, 1 equiv.) in THE (5 mL) and H₂O (5 mL) was added lithium hydroxide (0.12 g, 5.030 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 392.21, found 392.39.

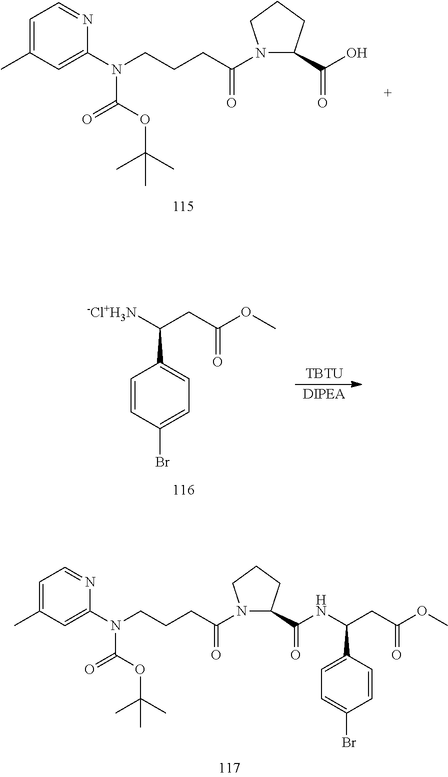

To a solution of compound 115 (300 mg, 0.766 mmol, 1 equiv.), compound 116 (237 mg, 0.804 mmol, 1.05 equiv.), and TBTU (295 mg, 0.919 mmol, 1.2 equiv.) in anhydrous DMF (10 mL) was added diisopropylethylamine (0.400 mL, 2.299 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×10 mL). The organic phase was combined, dried over Na₂SO₄, and concentrated. The product was purified by CombiFlash® using silica gel as the stationary phase and was eluted with 3-4% methanol in DCM. The yield is 83%. LC-MS: calculated [M+H]+ 631.21, found 631.46.

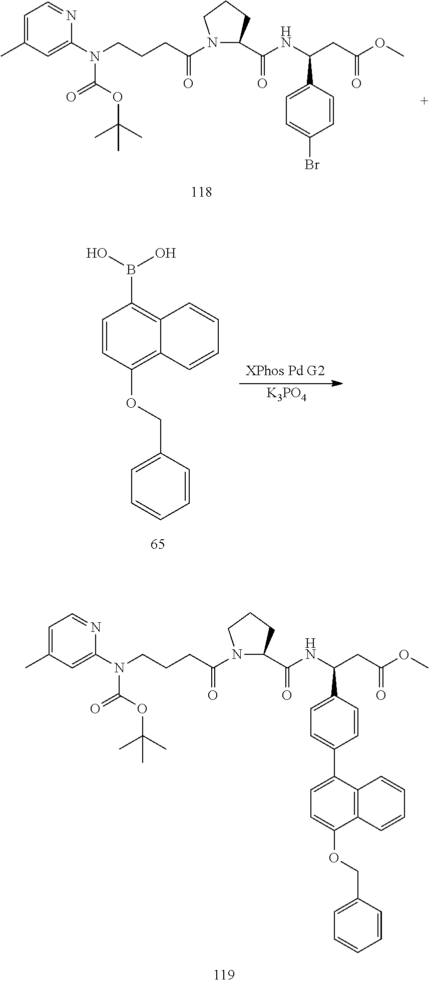

Compound 118 (100 mg, 0.158 mmol, 1.0 equiv.), compound 65 (66 mg, 0.237 mmol, 1.5 equiv.), XPhos Pd G2 (2.5 mg, 0.0032 mmol, 0.02 equiv.), and K₃PO₄ (67 mg, 0.316 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 20 min and the reaction was kept at 40° C. for 1 hr. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 3% methanol in DCM. The yield was 96%. LC-MS: calculated [M+H]+ 785.38, found 785.69.

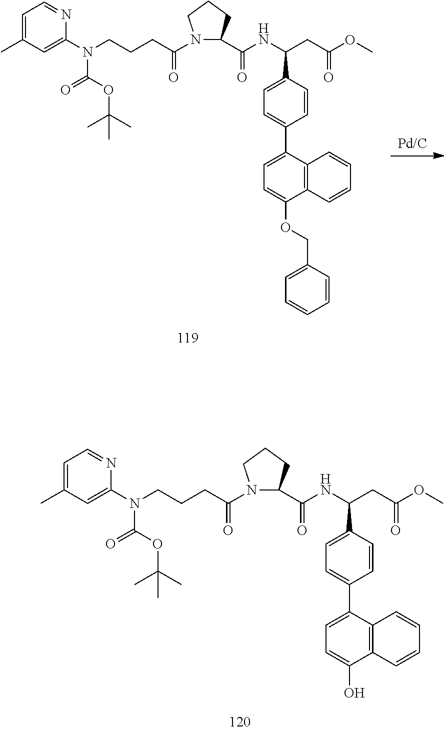

To a solution of compound 119 (0.120 g, 0.153 mmol, 1 equiv.) in ethyl acetate (10 mL) was added 10% Pd/C (100 mg) at room temperature. The reaction mixture was stirred at room temperature for overnight. The catalyst was removed by filtration through Celite® and the product was used directly without further purification. LC-MS: calculated [M+H]+ 695.34, found 695.66.

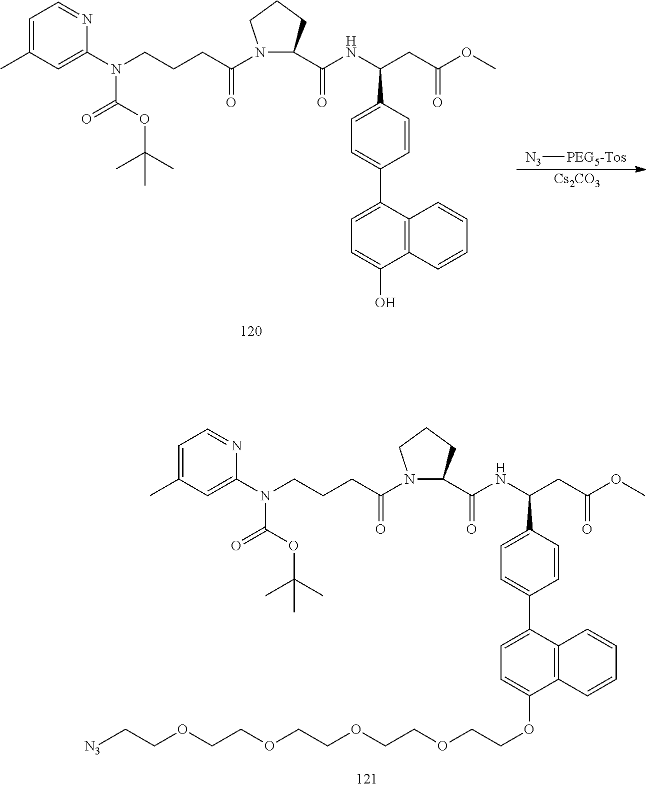

To a solution of compound 120 (83 mg, 0.119 mmol, 1 equiv.) and azido-PEG₅-OTs (100 mg, 0.239 mmol, 2 equiv.) in anhydrous DMF (2 mL) was added Cs₂CO₃ (78 mg, 0.239 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4% methanol in DCM. The yield was 79%. LC-MS: calculated 940.47, found 941.16.

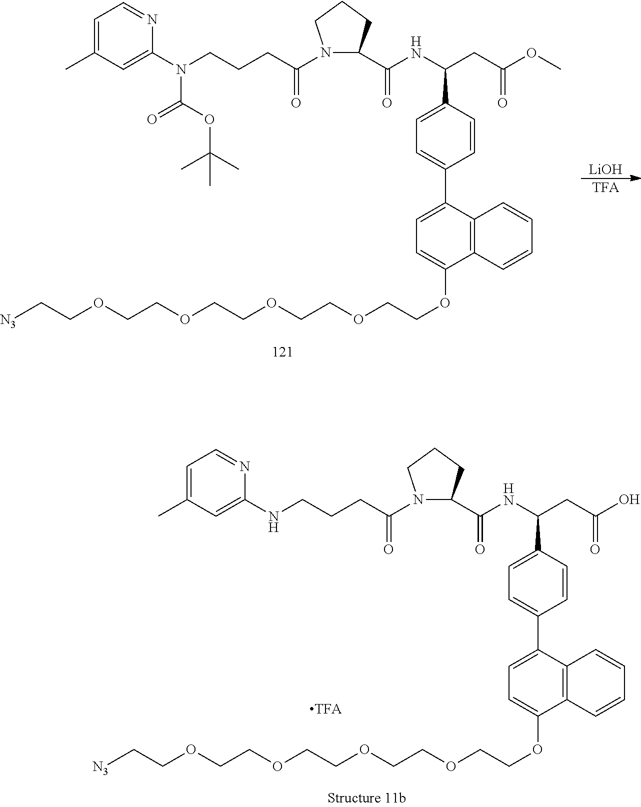 [SMILES: COC(=O)C[C@H](NC(=O)[C@@H]1CCCN1C(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)N2CCC[C@H]2C(=O)N[C@@H](CC(=O)O)c2ccc(-c3ccc(OCCOCCOCCOCCOCCN=[N+]=[N-])c4ccccc34)cc2)c1 ; "TFA"]

To a solution of compound 121 (89 mg, 0.0947 mmol, 1.0 equiv.) in THE (2 mL) and water (2 mL) was added lithium hydroxide (6.8 mg, 0.284 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 826.41, found 827.10.

### Synthesis of Structure 12b ((S)-3-(4-(4-((14-azido-3,6,9,12-tetraoxatetradecyl)oxy)benzo[d]oxazol-7-yl)phenyl)-3-(2-(4-((4-methylpyridin-2-yl)amino)butanamido)acetamido)propanoic acid)

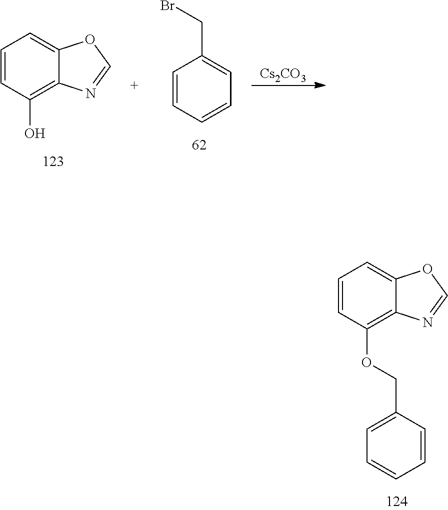

To a solution of compound 123 (1.0 g, 7.40 mmol, 1 equiv.), and compound 62 (1.32 mL, 11.10 mmol, 1.5 equiv.) in anhydrous DMF (10 mL) was added Cs₂CO₃ (3.62 g, 11.10 mmol, 1.5 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase and was eluted with 5-7% ethyl acetate in hexane. 85% yield.

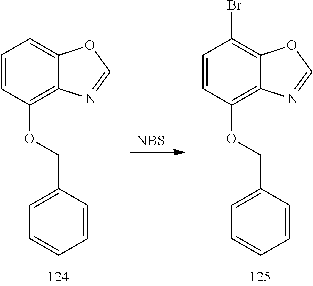

To a solution of compound 124 (1.425 g, 6.326 mmol, 1 equiv.) in anhydrous acetonitrile (20 mL) was added N-bromosuccinimide (1.216 g, 6.832 mmol, 1.08 equiv.) at 0° C. portion-wise. The reaction mixture was kept at 0° C. for another 30 min and then allowed to warm to room temperature and stirred overnight. The solvent was removed under reduced pressure and the residue was purified by CombiFlash® using silica gel as the stationary phase. The product was eluted with 4-5% ethyl acetate in hexane. 65% yield. LC-MS: calculated [M+H]+ 303.99. found 304.08.

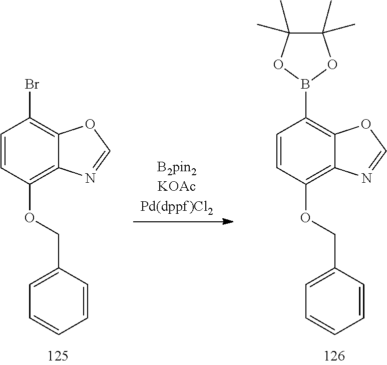 [SMILES: Brc1ccc(OCc2ccccc2)c2ncoc12 ; CC1(C)OB(c2ccc(OCc3ccccc3)c3ncoc23)OC1(C)C]

The mixture of compound 125 (1.339 g, 4.402 mmol, 1 equiv.), bis(pinacolato)diboron (2.236 g, 8.805 mmol, 2 equiv.), potassium acetate (0.864 g, 8.805 mmol, 2 equiv.) and Pd(dppf)Cl₂(161 mg, 0.220 mmol, 0.05 equiv.) in 15 mL of anhydrous 1,4-dioxane was stirred at 100° C. under nitrogen for 8 hours. After concentration, the residue was partitioned between H₂O and DCM, the aqueous phase was extracted with DCM, and the combined organic layer was washed with brine, dried over Na₂SO₄, and concentrated. The product was purified by CombiFlash® using silica gel as the stationary phase and was eluted with 15-20% ethyl acetate in hexane. LC-MS: calculated [M+H]+ 352.16, found 352.06.

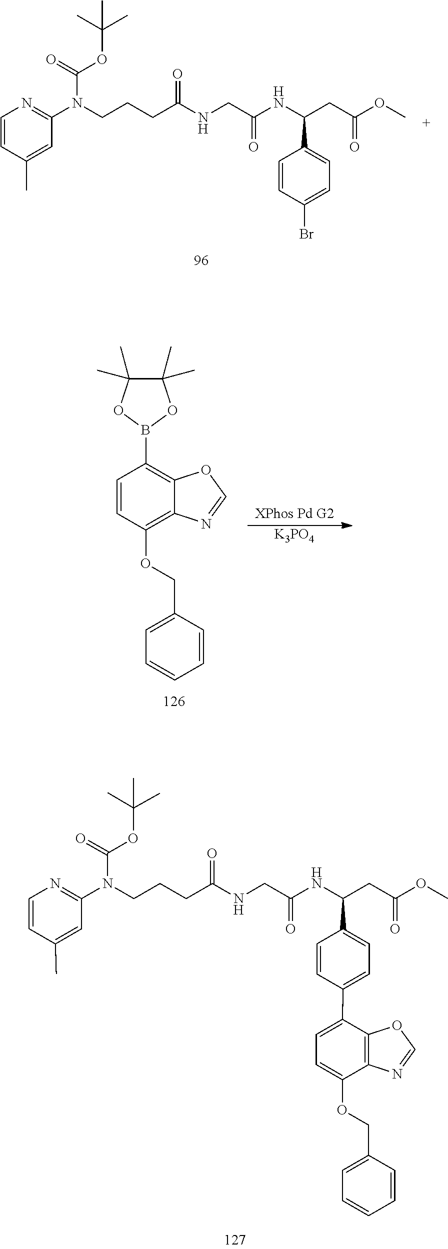

Compound 96 (200 mg, 0.338 mmol, 1.0 equiv.), compound 126 (178 mg, 0.507 mmol, 1.5 equiv.), XPhos Pd G2 (5.3 mg, 0.0068 mmol, 0.02 equiv.), and K₃PO₄ (143 mg, 0.676 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 20 min and the reaction was kept at 40° C. for 1 hr. The reaction was quenched with saturated NaHCO₃(10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 2-3% methanol in DCM. LC-MS: calculated [M+H]+ 736.33, found 736.89.

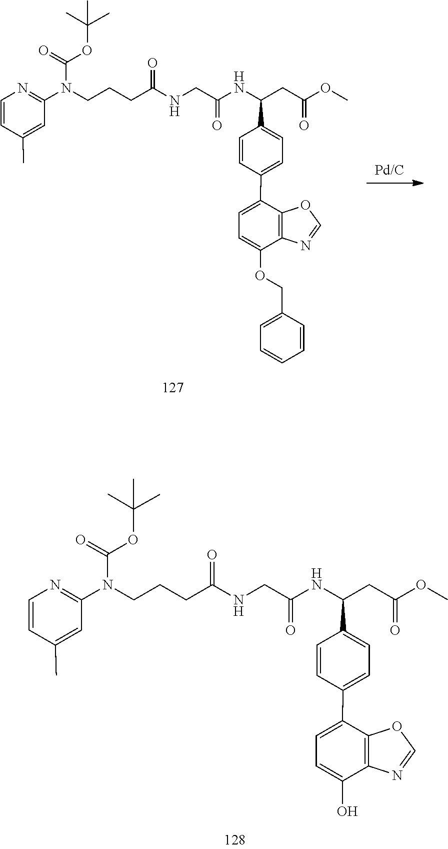 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ncoc23)cc1 ; COC(=O)C[C@H](NC(=O)CNC(=O)CCCN(C(=O)OC(C)(C)C)c1cc(C)ccn1)c1ccc(-c2ccc(O)c3ncoc23)cc1]

To a solution of compound 127 (0.219 g, 0.297 mmol, 1 equiv.) in ethyl acetate (10 mL) was added 10% Pd/C (100 mg) at room temperature. The reaction mixture was stirred at room temperature overnight. The catalyst was removed by filtration through Celite® and the product was used directly without further purification. LC-MS: calculated [M+H]+ 646.28, found 646.78.

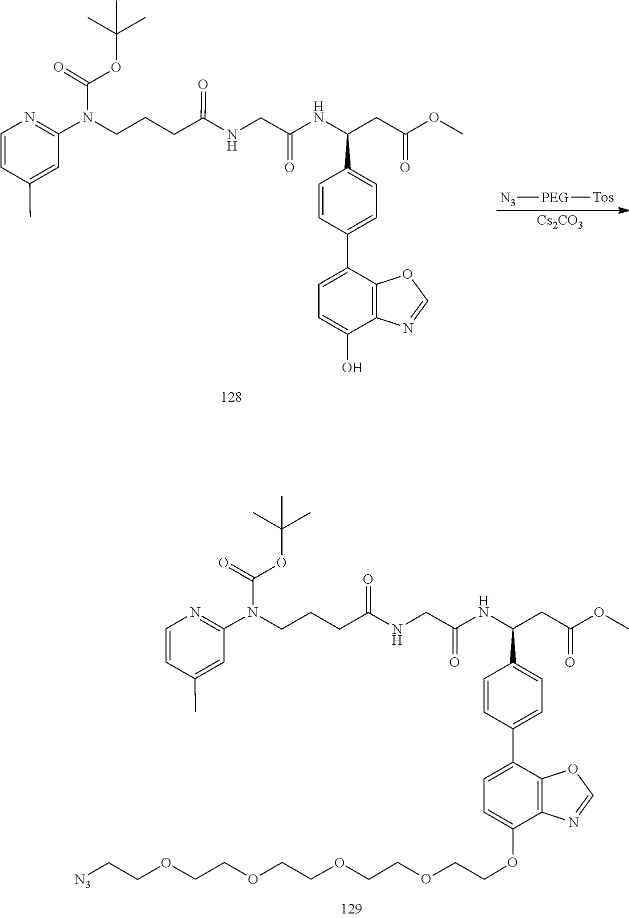

To a solution of compound 128 (73 mg, 0.113 mmol, 1 equiv.) and azido-PEG₅-OTs (94 mg, 0.226 mmol, 2 equiv.) in anhydrous DMF (2 mL) was added Cs₂CO₃ (74 mg, 0.226 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4% methanol in DCM. The yield is 80%. LC-MS: calculated [M+H]+ 891.42, found 892.00.

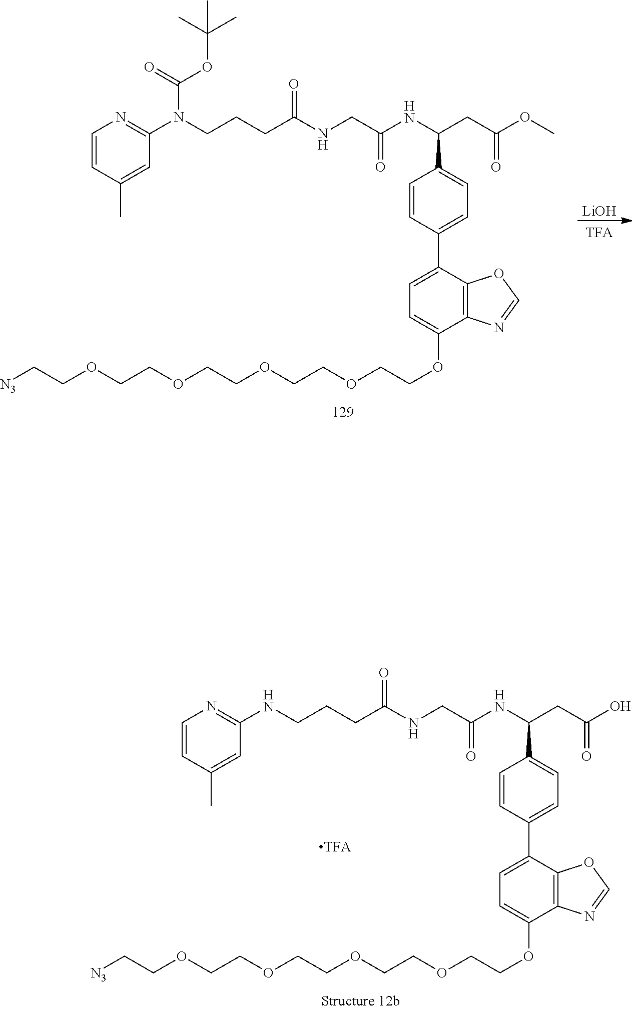 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3ncoc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4ncoc34)cc2)c1]

To a solution of compound 129 (43 mg, 0.0478 mmol, 1.0 equiv.) in THE (2 mL) and water (2 mL) was added lithium hydroxide (3.4 mg, 0.143 mmol, 3.0 equiv.) at room temperature. The mixture was stirred at room temperature for 1 hour. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 777.35, found 777.94.

### Synthesis of Structure 13b ((S)-3-(4-(4-((14-azido-3,6,9,12-tetraoxatetradecyl)oxy)-5,6,7,8-tetrahydronaphthalen-1-yl)phenyl)-3-(2-(4-((4-methylpyridin-2-yl)amino)butanamido)acetamido)propanoic acid)

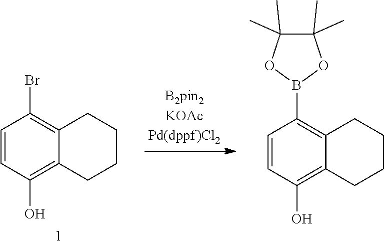 [SMILES: Oc1ccc(Br)c2c1CCCC2 ; CC1(C)OB(c2ccc(O)c3c2CCCC3)OC1(C)C]

The mixture of compound 1 (300 mg, 1.321 mmol, 1 equiv.), bis(pinacolato)diboron (671 mg, 2.642 mmol, 2 equiv.), potassium acetate (389 mg, 3.963 mmol, 2 equiv.) and Pd(dppf)Cl₂(48 mg, 0.066 mmol, 0.05 equiv.) in 10 mL of anhydrous 1,4-dioxane was stirred at 80° C. under nitrogen overnight. After concentration, the residue was partitioned between H₂O and DCM, the aqueous phase was extracted with DCM, and the combined organic layer was washed with brine, dried over Na₂SO₄, and concentrated. The product was purified by CombiFlash® using silica gel as the stationary phase and was eluted with 10% ethyl acetate in hexane. LC-MS: calculated [M-H]− 273.17, found 273.29.

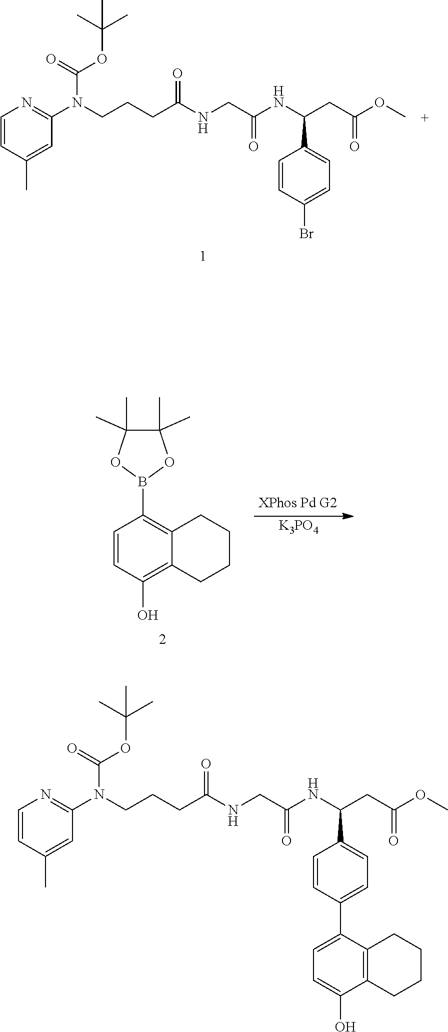

Compound 1 (100 mg, 0.169 mmol, 1.0 equiv.), compound 2 (70 mg, 0.253 mmol, 1.5 equiv.), XPhos Pd G2 (2.7 mg, 0.0034 mmol, 0.02 equiv.), and K₃PO₄ (72 mg, 0.338 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 20 min and the reaction was kept at 40° C. for 3 hr. The reaction was then cooled to room temperature and left overnight. The reaction was quenched with saturated NaHCO₃(10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 4-5% methanol in DCM. LC-MS: calculated [M+H]+ 659.34, found 659.57.

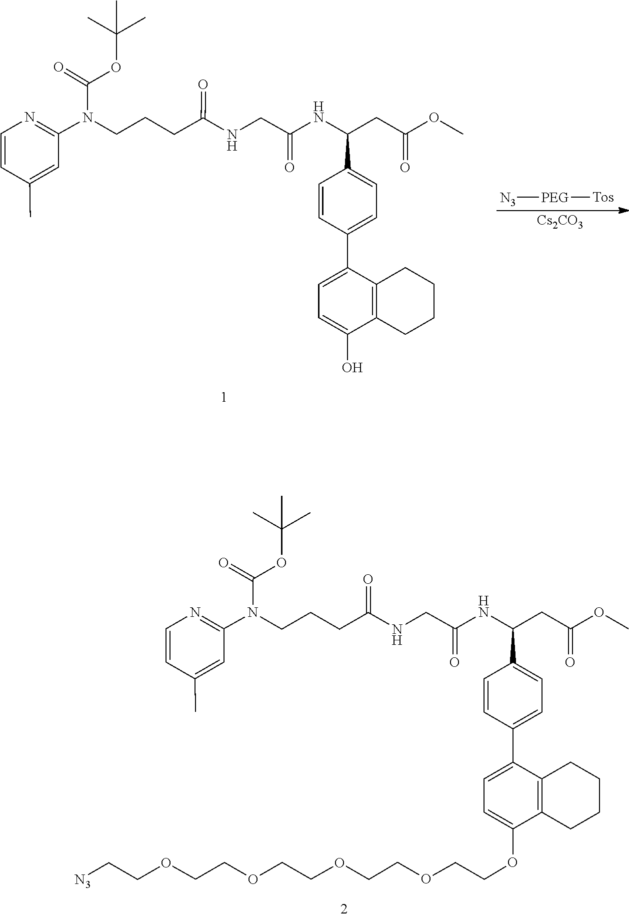

To a solution of compound 1 (30 mg, 0.0455 mmol, 1 equiv.) and azido-PEG₅-OTs (38 mg, 0.0911 mmol, 2 equiv.) in anhydrous DMF (2 mL) was added Cs₂CO₃ (30 mg, 0.0911 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4% methanol in DCM. The yield is 70%. LC-MS: calculated [M+H]+ 904.47, found 904.88.

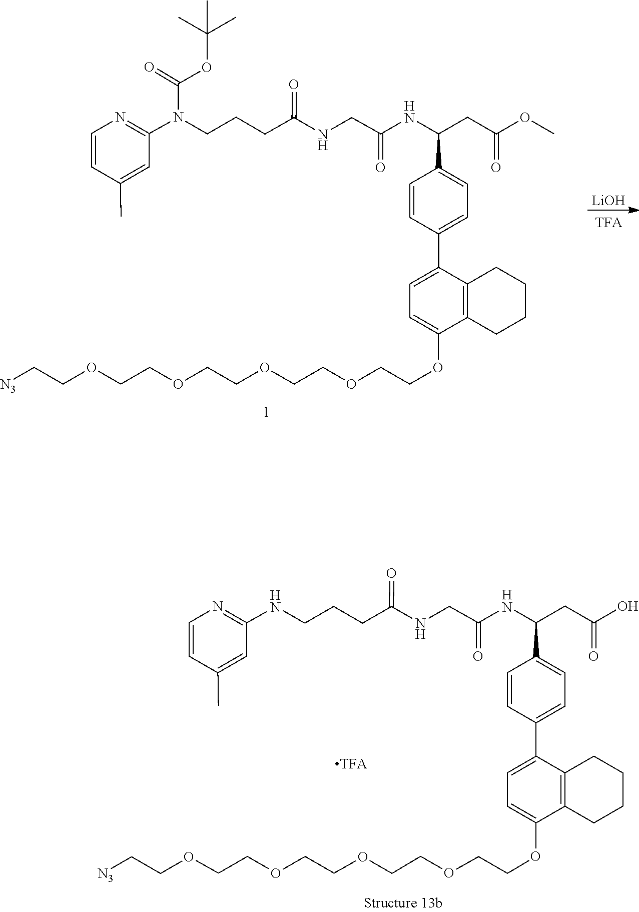 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3c2CCCC3)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4c3CCCC4)cc2)c1]

To a solution of compound 1 (29 mg, 0.0321 mmol, 1.0 equiv.) in THE (2 mL) and water (2 mL) was added lithium hydroxide (2.3 mg, 0.0962 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) were added into the residue and the mixture was stirred at room temperature for another 3 hr. The solvent was removed by rotary evaporator. LC-MS: calculated [M+H]+ 790.41, found 790.64.

### Synthesis of Structure 14b ((S)-3-(4′-((14-azido-3,6,9,12-tetraoxatetradecyl)oxy)-2′-(trifluoromethoxy)-[1,1′-biphenyl]-4-yl)-3-(2-(4-((4-methylpyridin-2-yl)amino)butanamido)acetamido)propanoic acid)

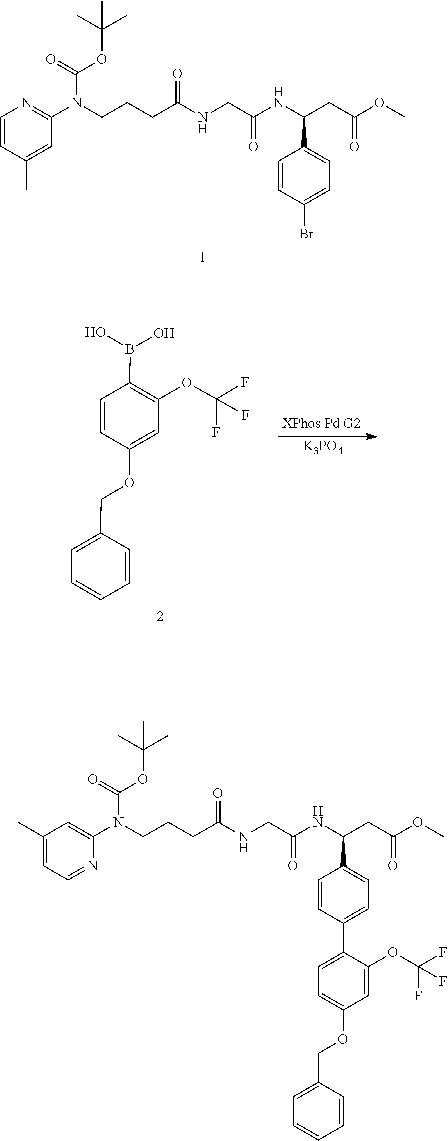

Compound 1 (150 mg, 0.253 mmol, 1.0 equiv.), compound 2 (118 mg, 0.380 mmol, 1.5 equiv.), XPhos Pd G2 (4 mg, 0.0051 mmol, 0.02 equiv.), and K₃PO₄ (107 mg, 0.507 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 10 min and the reaction was kept at 40° C. for overnight. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 2-4% methanol in DCM. LC-MS: calculated [M+H]+ 779.32, found 779.65.

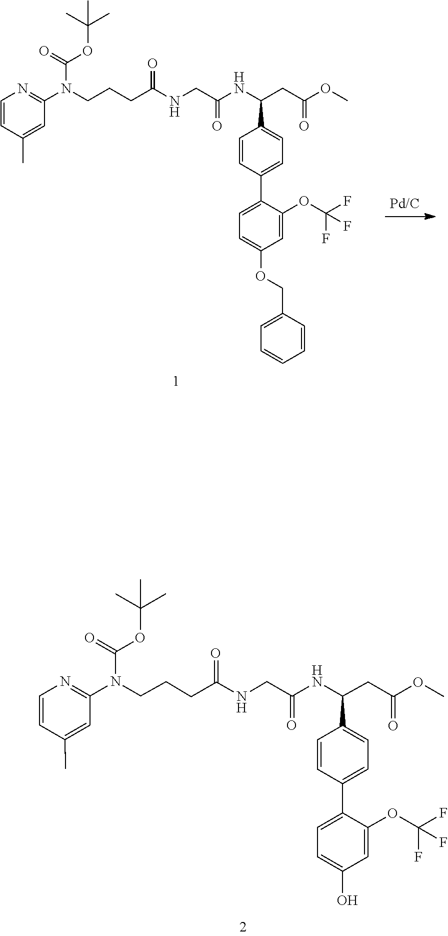 [SMILES: COC(=O)C[C@H](NC(=O)CNC(=O)CCCN(C(=O)OC(C)(C)C)c1cc(C)ccn1)c1ccc(-c2ccc(OCc3ccccc3)cc2OC(F)(F)F)cc1 ; COC(=O)C[C@H](NC(=O)CNC(=O)CCCN(C(=O)OC(C)(C)C)c1cc(C)ccn1)c1ccc(-c2ccc(O)cc2OC(F)(F)F)cc1]

To a solution of compound 1 (0.19 g, 0.244 mmol, 1 equiv.) in ethyl acetate (10 mL) was added 10% Pd/C (100 mg) at room temperature. The reaction was evacuated and backfilled with hydrogen (this process was repeated for 3 times). The reaction mixture was stirred at room temperature for overnight. The catalyst was removed by filtration through Celite® and the product was used directly without further purification. LC-MS: calculated [M+H]+ 689.27, found 689.54.

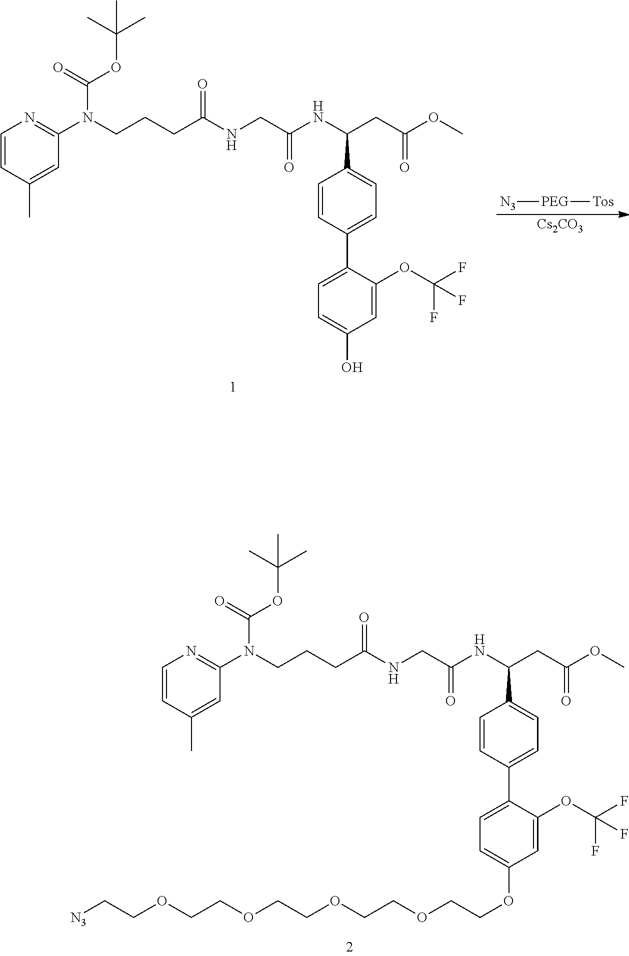

To a solution of compound 1 (80 mg, 0.116 mmol, 1 equiv.) and azido-PEG₅-OTs (97 mg, 0.232 mmol, 2 equiv.) in anhydrous DMF (2 mL) was added Cs₂CO₃ (76 mg, 0.232 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. The yield was 82%. LC-MS: calculated [M+H]+ 934.41, found 935.04.

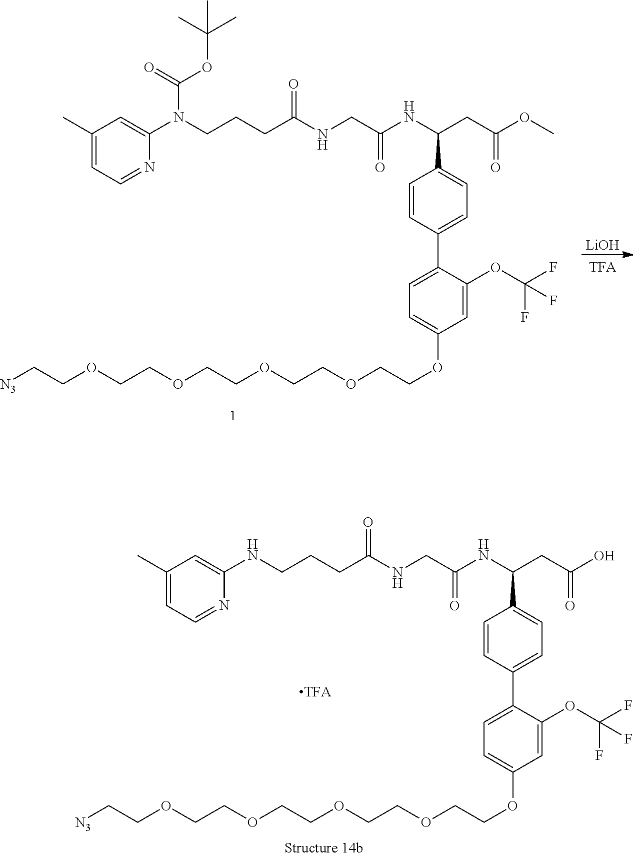

To a solution of compound 1 (90 mg, 0.0964 mmol, 1.0 equiv.) in THE (2 mL) and water (2 mL) was added lithium hydroxide (7 mg, 0.289 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 820.34, found 820.89.

### Synthesis of Structure 15b ((S)-3-(3-(5-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

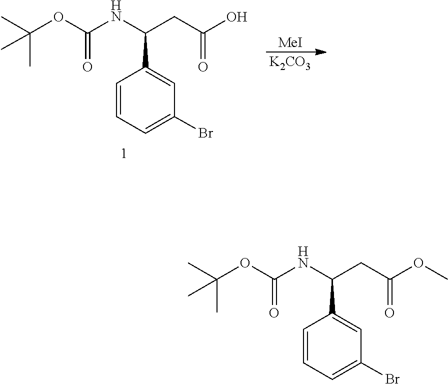

To a solution of compound 1 (1.0 g, 2.90 mmol, 1 equiv.) and potassium carbonate (0.60 g, 4.36 mmol, 1.5 equiv.) in anhydrous DMF (10 mL) was added methyl iodide (362 uL, 5.81 mmol, 2.0 equiv.) at room temperature. The reaction mixture was stirred at room temperature for 1 hr. The reaction was then quenched with water (20 mL) and the aqueous phase was extracted with ethyl acetate (3×10 mL). The organic phase was combined, dried over anhydrous Na₂SO₄, and concentrated. The product was separated by CombiFlash® using silica gel as the stationary phase and was eluted with 15% ethyl acetate in hexane. LC-MS: calculated [M+H]+ 358.06, found 358.18.

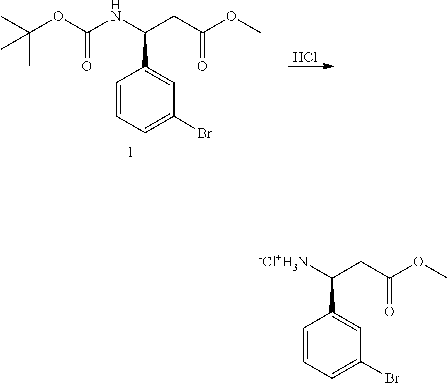

Compound 1 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 258.01, found 258.08.

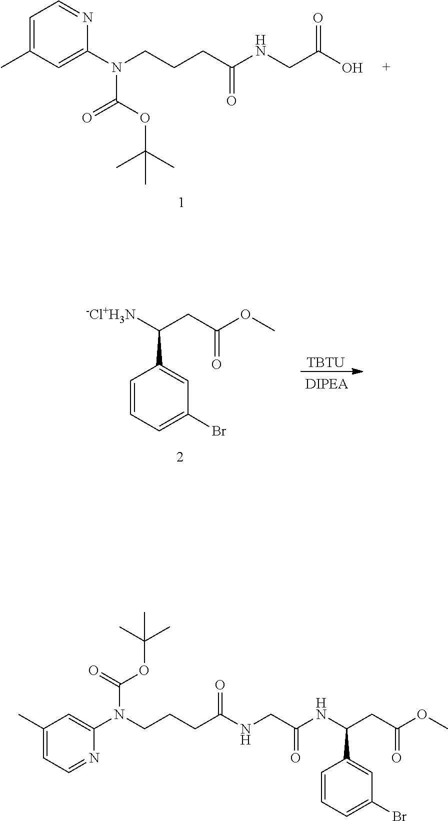

To a solution of compound 1 (640 mg, 1.821 mmol, 1 equiv.), compound 2 (590 mg, 2.003 mmol, 1.10 equiv.), and TBTU (702 mg, 2.185 mmol, 1.20 equiv.) in anhydrous DMF (10 mL) was added diisopropylethylamine (0.952 mL, 5.464 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×10 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 591.17, found 591.40.

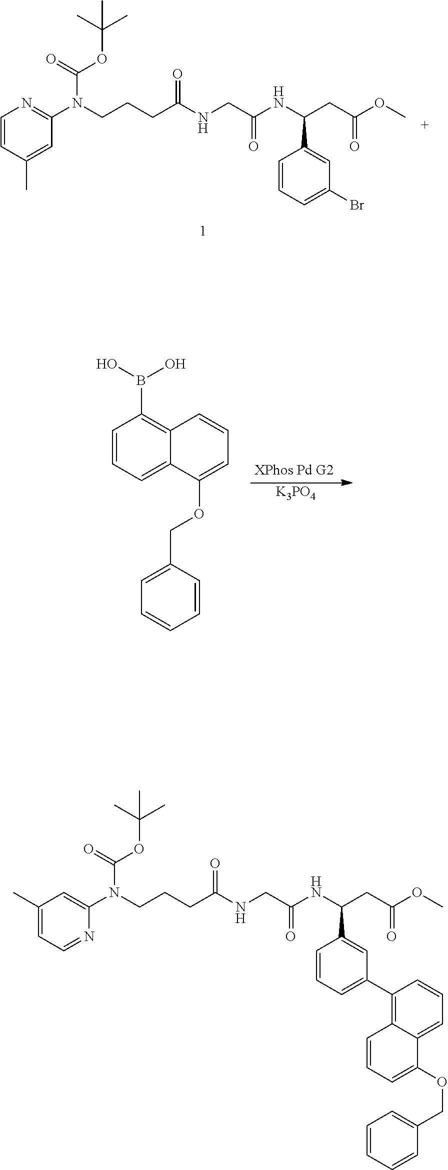

Compound 1 (150 mg, 0.253 mmol, 1.0 equiv.), compound 2 (106 mg, 0.380 mmol, 1.5 equiv.), XPhos Pd G2 (4 mg, 0.0051 mmol, 0.02 equiv.), and K₃PO₄ (107 mg, 0.507 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 10 min and the reaction was kept at 40° C. for 2 hours. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 3-4% methanol in DCM. LC-MS: calculated [M+H]+ 745.35, found 745.99.

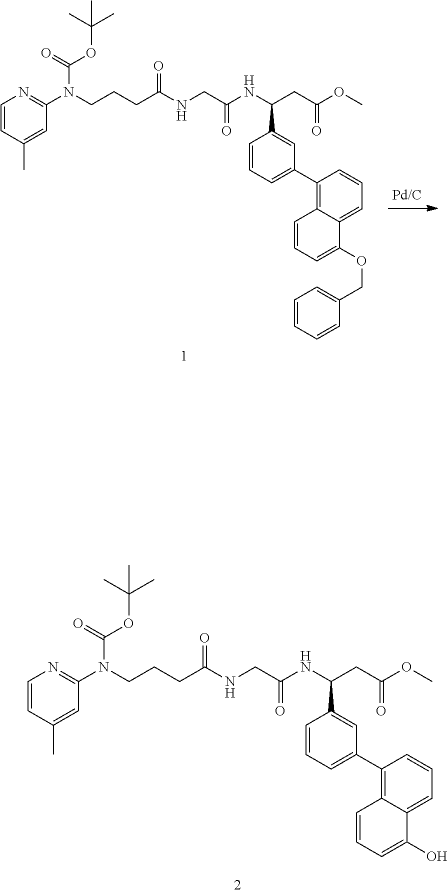 [SMILES: COC(=O)C[C@H](NC(=O)CNC(=O)CCCN(C(=O)OC(C)(C)C)c1cc(C)ccn1)c1cccc(-c2cccc3c(OCc4ccccc4)cccc23)c1 ; COC(=O)C[C@H](NC(=O)CNC(=O)CCCN(C(=O)OC(C)(C)C)c1cc(C)ccn1)c1cccc(-c2cccc3c(O)cccc23)c1]

To a solution of compound 1 (0.189 g, 0.253 mmol, 1 equiv.) in ethyl acetate (10 mL) was added 10% Pd/C (100 mg) at room temperature. The reaction was evacuated and backfilled with hydrogen (this process was repeated for 3 times.). The reaction mixture was stirred at room temperature for overnight. The catalyst was removed by filtration through Celite® and the product was used directly without further purification. LC-MS: calculated [M+H]+ 655.31, found 655.42.

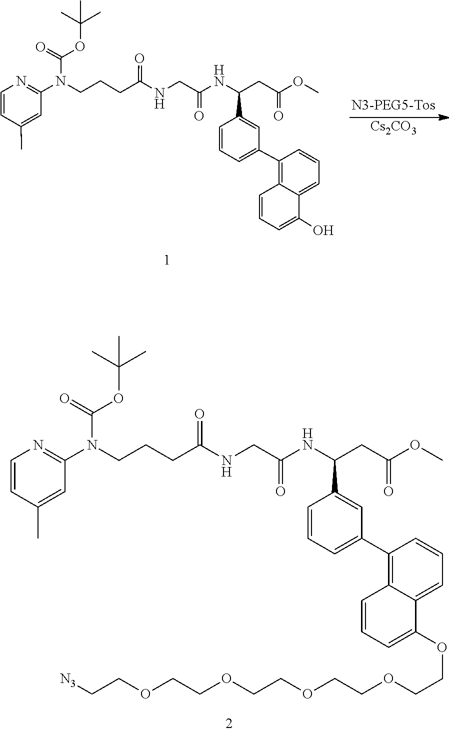

To a solution of compound 1 (80 mg, 0.122 mmol, 1 equiv.) and azido-PEG₅-OTs (102 mg, 0.244 mmol, 2 equiv.) in anhydrous DMF (2 mL) was added Cs₂CO₃ (80 mg, 0.244 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 1-2% methanol in DCM. The yield is 90%. LC-MS: calculated 900.44, found 901.10.

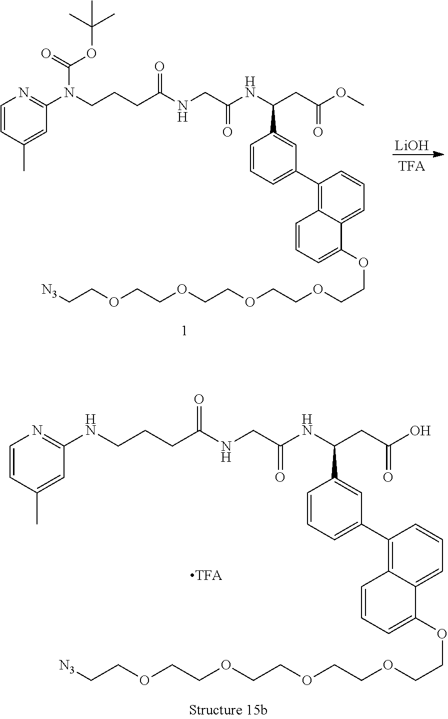 [SMILES: COC(=O)C[C@H](NC(=O)CNC(=O)CCCN(C(=O)OC(C)(C)C)c1cc(C)ccn1)c1cccc(-c2cccc3c(OCCOCCOCCOCCOCCN=[N+]=[N-])cccc23)c1 ; "LiOH" ; Cc1ccnc(NCCCC(=O)NCC(=O)N[C@@H](CC(=O)O)c2cccc(-c3cccc4c(OCCOCCOCCOCCOCCN=[N+]=[N-])cccc34)c2)c1]

To a solution of compound 1 (100 mg, 0.111 mmol, 1.0 equiv.) in THE (2 mL) and water (2 mL) was added lithium hydroxide (8 mg, 0.333 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 786.37, found 786.95.

### Synthesis of Structure 16b ((S)-3-(4-(4-((14-azido-3,6,9,12-tetraoxatetradecyl)oxy)naphthalen-1-yl)phenyl)-3-((R)-1-(4-((4-methylpyridin-2-yl)amino)butanoyl)pyrrolidine-2-carboxamido)propanoic acid)

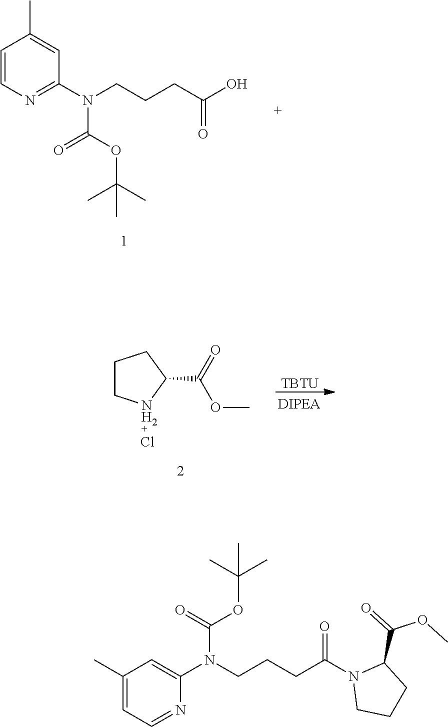

To a solution of compound 1 (500 mg, 1.698 mmol, 1 equiv.), compound 2 (295 mg, 1.783 mmol, 1.05 equiv.), and TBTU (654 mg, 2.038 mmol, 1.2 equiv.) in anhydrous DMF (10 mL) was added diisopropylethylamine (0.888 mL, 5.096 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×10 mL). The organic phase was combined, dried over Na₂SO₄, and concentrated. The product was purified by CombiFlash® using silica gel as the stationary phase and was eluted with 2-3% methanol in DCM. The yield is 98.43%. LC-MS: calculated [M+H]+ 406.23, found 406.34.

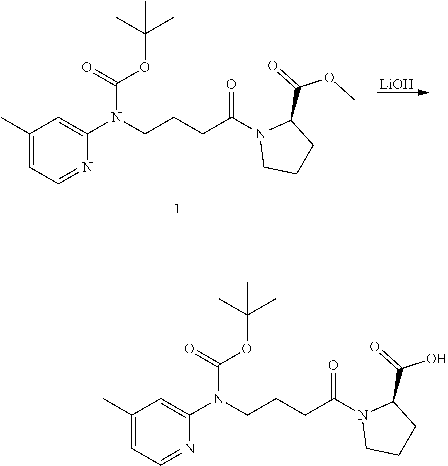

To a solution of compound 1 (0.678 g, 1.672 mmol, 1 equiv.) in THE (10 mL) and H₂O (10 mL) was added lithium hydroxide (0.12 g, 5.016 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 392.21, found 392.39.

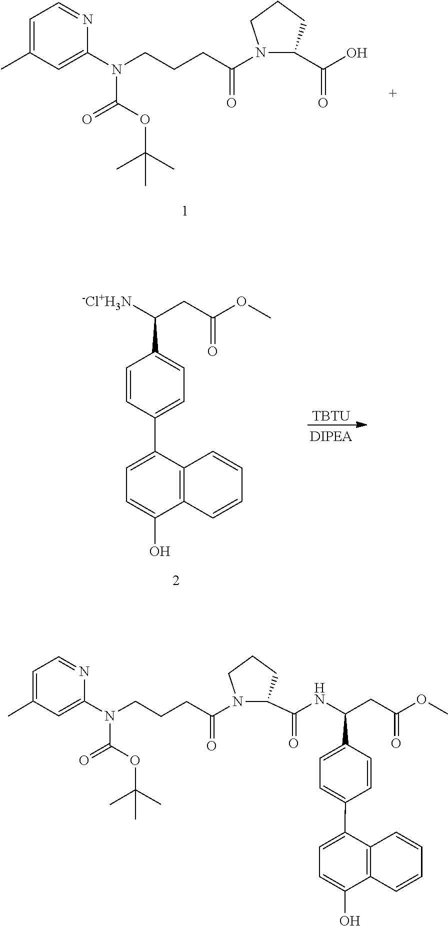

To a solution of compound 1 (130 mg, 0.332 mmol, 1 equiv.), compound 2 (125 mg, 0.348 mmol, 1.05 equiv.), and TBTU (128 mg, 0.398 mmol, 1.2 equiv.) in anhydrous DMF (5 mL) was added diisopropylethylamine (0.174 mL, 0.996 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×10 mL). The organic phase was combined, dried over Na₂SO₄, and concentrated. The product was purified by CombiFlash® using silica gel as the stationary phase and was eluted with 3-4% methanol in DCM. The yield is 86%. LC-MS: calculated [M+H]+ 695.34, found 695.93.

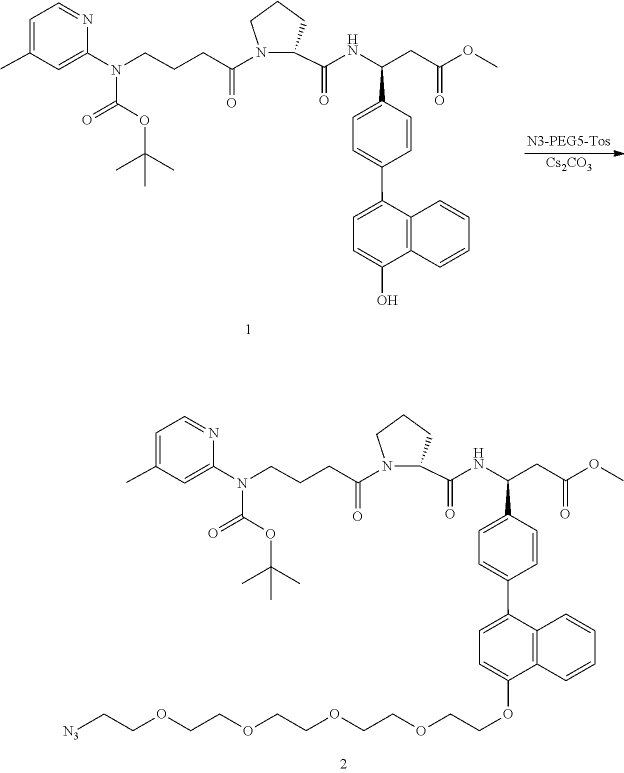

To a solution of compound 1 (80 mg, 0.115 mmol, 1 equiv.) and azido-PEG₅-OTs (96 mg, 0.230 mmol, 2 equiv.) in anhydrous DMF (2 mL) was added Cs₂CO₃ (75 mg, 0.230 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4-5% methanol in DCM. The yield is 60%.

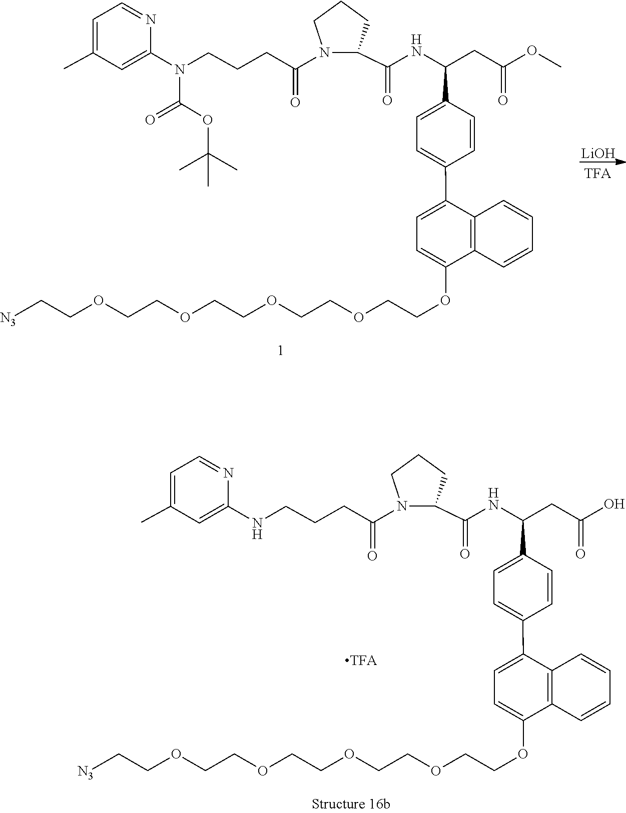 [SMILES: COC(=O)C[C@H](NC(=O)[C@H]1CCCN1C(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)N2CCC[C@@H]2C(=O)N[C@@H](CC(=O)O)c2ccc(-c3ccc(OCCOCCOCCOCCOCCN=[N+]=[N-])c4ccccc34)cc2)c1]

To a solution of compound 1 (65 mg, 0.0691 mmol, 1.0 equiv.) in THE (2 mL) and water (2 mL) was added lithium hydroxide (5 mg, 0.207 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator. LC-MS: calculated [M+H]+ 826.41, found 827.01.

### Synthesis of Structure 17b ((S)-3-(4-(7-((14-azido-3,6,9,12-tetraoxatetradecyl)oxy)benzo[b]thiophen-4-yl)phenyl)-3-(2-(4-((4-methylpyridin-2-yl)amino)butanamido)acetamido)propanoic acid)

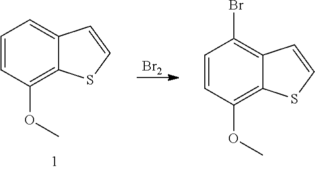

A solution of bromine (1.877 g, 11.745 mmol, 1.05 equiv.) in dry tetrachloromethane (20 mL) was added dropwise during 1.5 hours to a stirred solution of compound 1 (1.837 g, 11.186 mmol, 1 equiv.) in tetrachloromethane (20 mL) at 0° C. After a further hour at 0° C., the organic layer was washed with water and brine, dried over Na₂SO₄, concentrated to give a residue, which was purified by CombiFlash® using silica gel as the stationary phase. The product was eluted with pure hexane with impurities.

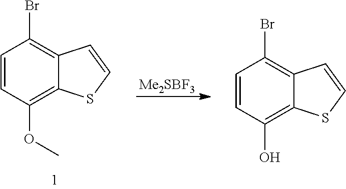

To a dichloromethane (20 ml) solution of compound 1 (2.70 g, 11.105 mmol, 1.0 equiv.), under nitrogen atmosphere, at 0° C., boron trifluoride dimethyl sulfide complex (3.5 mL, 33.317 mmol, 3.0 equiv.) was added and stirred at room temperature for 20 hours. The reaction mixture was cooled to 0° C. and quenched with saturated NH₄Cl solution (20 mL). The aqueous phase was extracted with ethyl acetate (3×20 mL) and the organic phase was combined, dried over Na₂SO₄, and concentrated. The product was separated by CombiFlash® using silica gel as the stationary phase and was eluted with 5% ethyl acetate in hexane. LC-MS: calculated [M-H]-226.92, found 227.03.

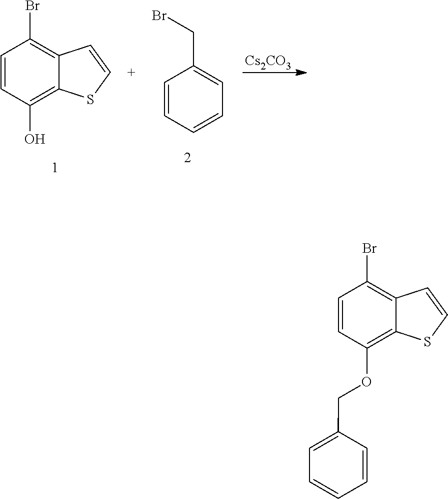

To a solution of compound 1 (1.838 g, 8.023 mmol, 1 equiv.), and compound 2 (1.906 mL, 16.04 mmol, 2 equiv.) in anhydrous DMF (10 mL) was added Cs₂CO₃ (5.228 g, 16.04 mmol, 2 equiv.) at room temperature. The reaction mixture was stirred at room temperature overnight. The reaction was quenched by water (20 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 2-3% ethyl acetate in hexane.

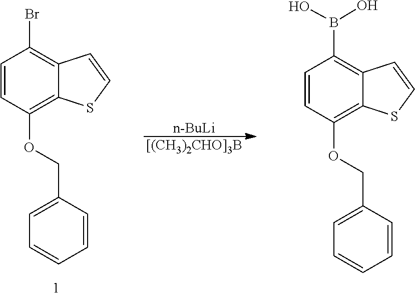

To a solution of compound 1 (2.22 g, 6.954 mmol, 1.0 equiv.) in anhydrous THF (20 mL) was added n-BuLi in hexane (4.17 mL, 10.43 mmol, 1.5 equiv.) drop-wise at −78° C. The reaction was kept at −78° C. for another 1 hr. Triisopropylborate (2.40 mL, 10.43 mmol, 1.5 equiv.) was then added into the mixture at −78° C. The reaction was then warmed up to room temperature and stirred for another 1 hr. The reaction was quenched by saturated NH₄C₁ solution (20 mL) and the pH was adjusted to 3. The aqueous phase was extracted with EtOAc (3×20 mL) and the organic phase was combined, dried over Na₂SO₄, and concentrated. The product was separated by CombiFlash® using silica gel as the stationary phase and was eluted with 4-6% methanol in DCM. LC-MS: calculated [M-H]− 283.07, found 283.20.

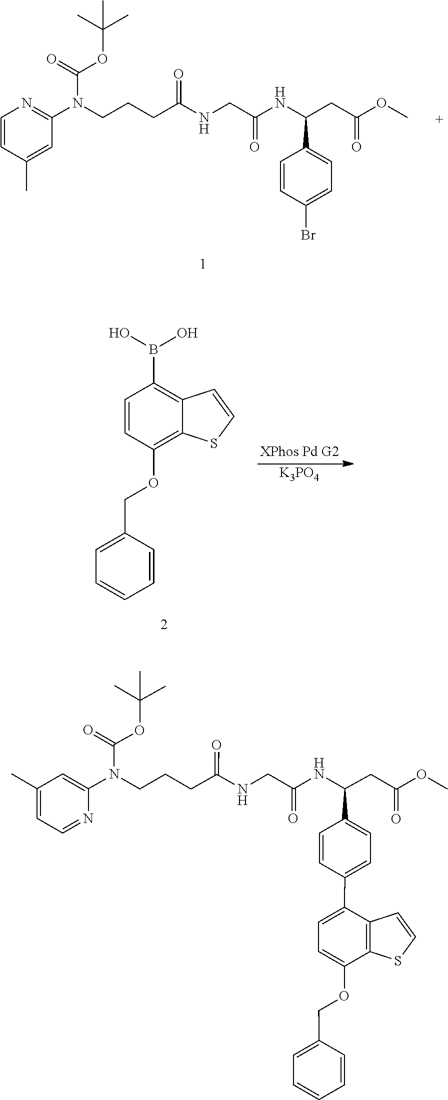

Compound 1 (400 mg, 0.676 mmol, 1.0 equiv.), compound 2 (288 mg, 1.01 mmol, 1.5 equiv.), XPhos Pd G2 (10 mg, 0.0135 mmol, 0.02 equiv.), and K₃PO₄ (287 mg, 1.352 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 10 min and the reaction was kept at 40° C. for 2 hours. The reaction was quenched with saturated NaHCO₃ solution (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 3-4% methanol in DCM. LC-MS: calculated [M+H]+ 751.31, found 751.84.

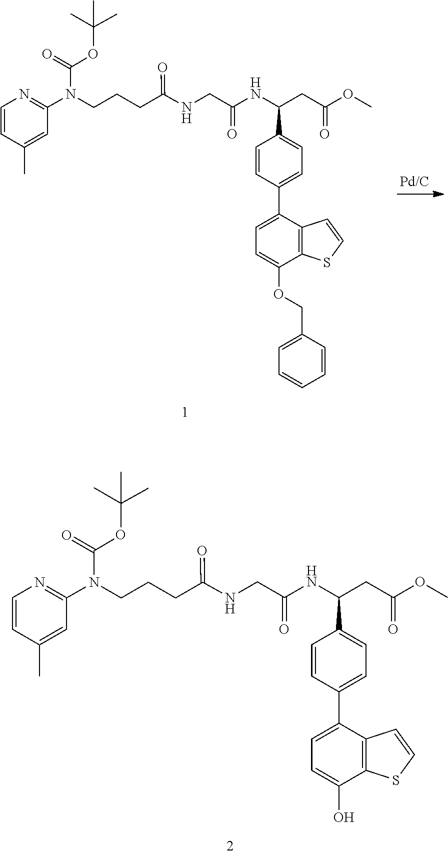 [SMILES: COC(=O)C[C@H](NC(=O)CNC(=O)CCCN(C(=O)OC(C)(C)C)c1cc(C)ccn1)c1ccc(-c2ccc(OCc3ccccc3)c3sccc23)cc1 ; COC(=O)C[C@H](NC(=O)CNC(=O)CCCN(C(=O)OC(C)(C)C)c1cc(C)ccn1)c1ccc(-c2ccc(O)c3sccc23)cc1]

To a solution of compound 1 (0.50 g, 0.666 mmol, 1 equiv.) in ethyl acetate (10 mL) was added 10% Pd/C (100 mg) at room temperature. The reaction was evacuated and backfilled with hydrogen (this process was repeated for 3 times.). The reaction mixture was stirred at room temperature for overnight. The catalyst was removed by filtration through Celite® and the product was separated by CombiFlash® using silica gel as the stationary phase and was eluted with 5% methanol in DCM. LC-MS: calculated [M+H]+ 661.26, found 661.73.

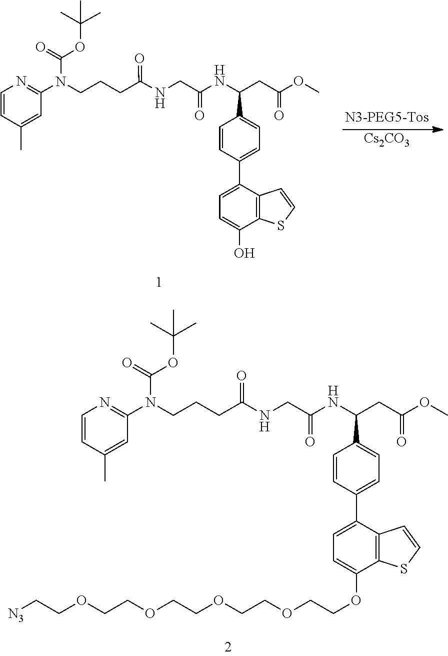

To a solution of compound 1 (130 mg, 0.196 mmol, 1 equiv.) and azido-PEG₅-OTs (164 mg, 0.393 mmol, 2 equiv.) in anhydrous DMF (2 mL) was added Cs₂CO₃ (128 mg, 0.393 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. The yield is 82%. LC-MS: calculated [M+H]+ 906.40, found 906.95.

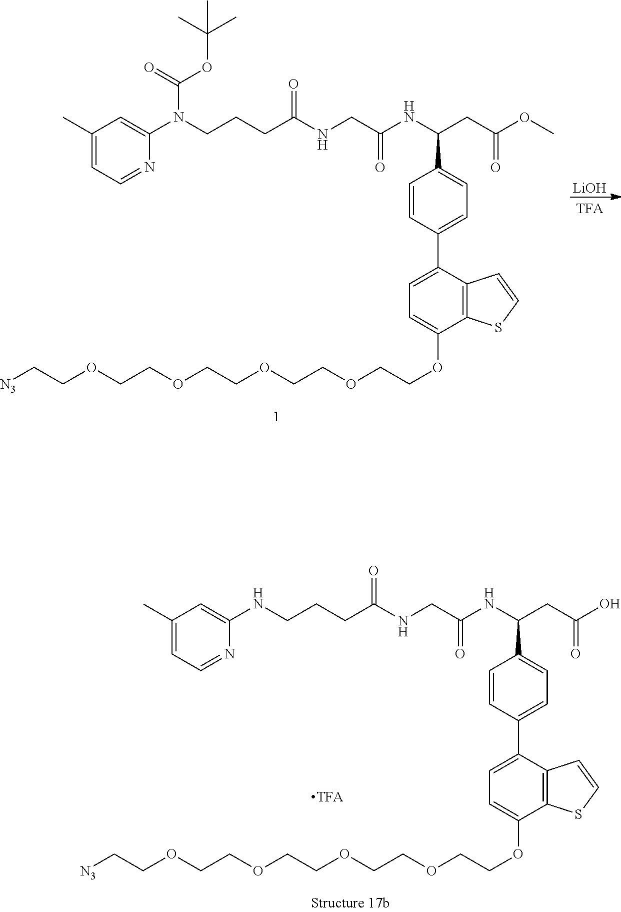 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3sccc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4sccc34)cc2)c1]

To a solution of compound 1 (147 mg, 0.162 mmol, 1.0 equiv.) in THE (2 mL) and water (2 mL) was added lithium hydroxide (12 mg, 0.486 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (2 mL) and DCM (2 mL) was added into the residue and the mixture was stirred at room temperature for another 3 hr. The solvent was removed by rotary evaporator and the product was separated by CombiFlash® using silica gel as the stationary phase. LC-MS: calculated [M+H]+ 792.33, found 792.89.

### Synthesis of Structure 18b ((S)-3-(4-(6-((14-azido-3,6,9,12-tetraoxatetradecyl)oxy)naphthalen-2-yl)phenyl)-3-(2-(4-((4-methylpyridin-2-yl)amino)butanamido)acetamido)propanoic acid)

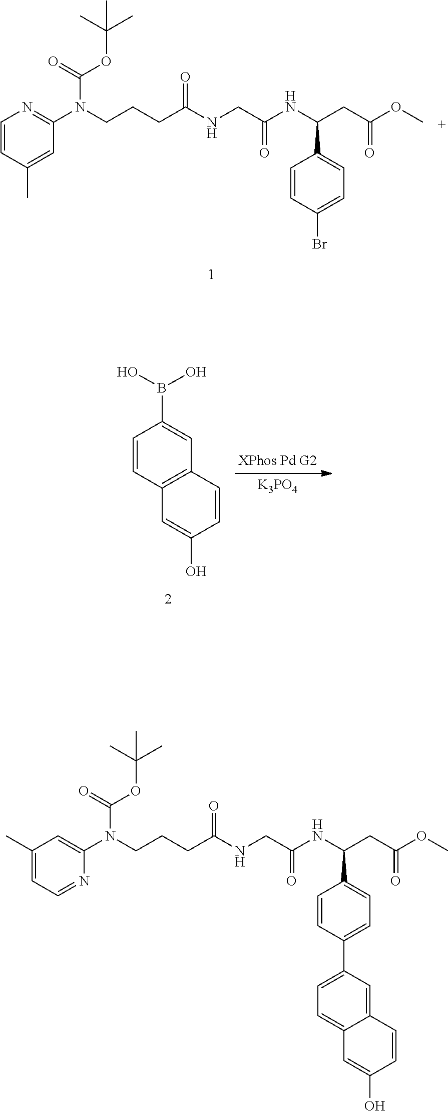

Compound 1 (150 mg, 0.253 mmol, 1.0 equiv.), compound 2 (71.5 mg, 0.380 mmol, 1.5 equiv.), XPhos Pd G2 (4 mg, 0.0051 mmol, 0.02 equiv.), and K₃PO₄ (107 mg, 0.507 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 10 min and the reaction was kept at 40° C. for 2 hours. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 2-3% methanol in DCM. LC-MS: calculated [M+H]+ 655.31, found 655.87.

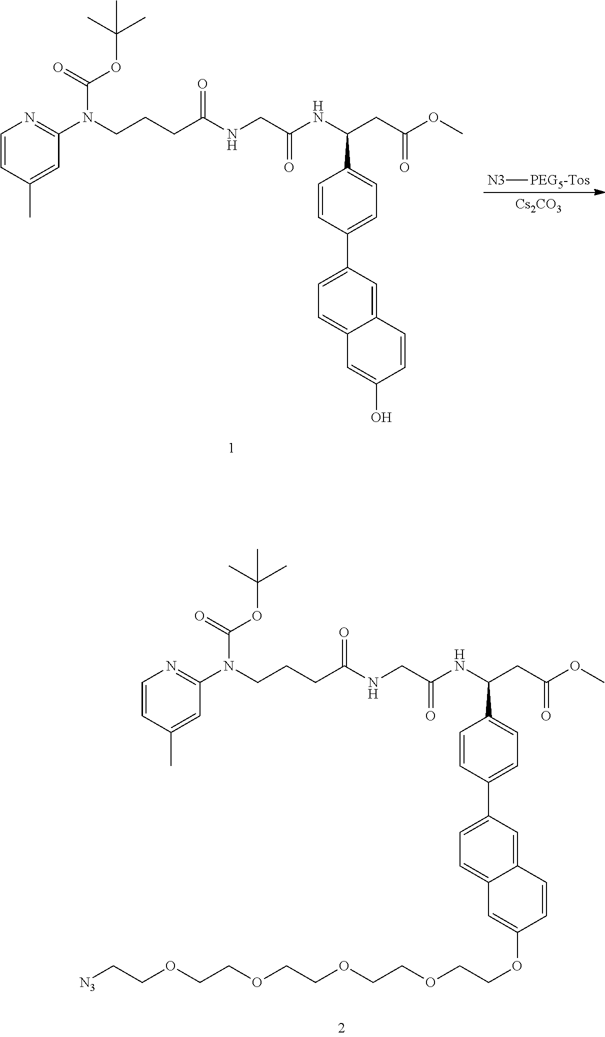

To a solution of compound 1 (160 mg, 0.244 mmol, 1 equiv.) and azido-PEG₅-OTs (204 mg, 0.488 mmol, 2 equiv.) in anhydrous DMF (2 mL) was added Cs₂CO₃ (160 mg, 0.488 mmol, 2 equiv.) at room temperature. The reaction mixture was stirred for 3 hours at 60° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. The yield was 30%. LC-MS: calculated [M+H]+ 900.44, found 901.01.

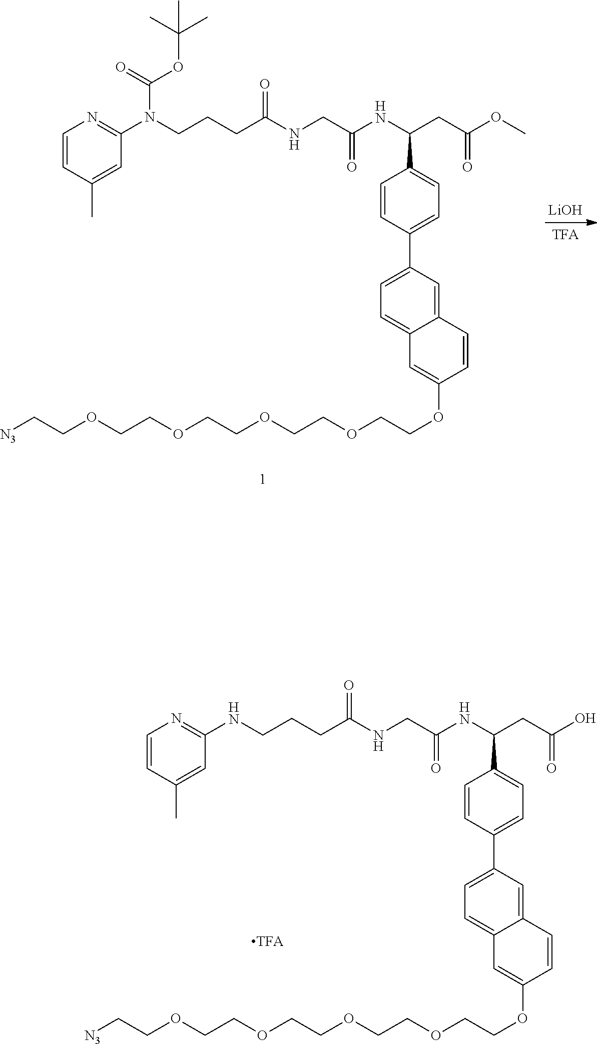 [SMILES: COC(=O)C[C@H](NC(=O)CNC(=O)CCCN(C(=O)OC(C)(C)C)c1cc(C)ccn1)c1ccc(-c2ccc3cc(OCCOCCOCCOCCOCCN=[N+]=[N-])ccc3c2)cc1 ; "LiOH" ; Cc1ccnc(NCCCC(=O)NCC(=O)N[C@@H](CC(=O)O)c2ccc(-c3ccc4cc(OCCOCCOCCOCCOCCN=[N+]=[N-])ccc4c3)cc2)c1]

To a solution of compound 1 (67 mg, 0.0744 mmol, 1.0 equiv.) in THF (2 mL) and water (2 mL) was added lithium hydroxide (5 mg, 0.223 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (2 mL) and DCM (2 mL) was added into the residue and the mixture was stirred at room temperature for another 3 hr. The solvent was removed by rotary evaporator and the product was separated by CombiFlash® using silica gel as the stationary phase and eluted with 10% methanol in DCM. LC-MS: calculated [M+H]+ 786.37, found 786.86.

### Synthesis of Structure 19b ((S)-3-(3-(6-((14-azido-3,6,9,12-tetraoxatetradecyl)oxy)naphthalen-2-yl)phenyl)-3-(2-(4-((4-methylpyridin-2-yl)amino)butanamido)acetamido)propanoic acid)

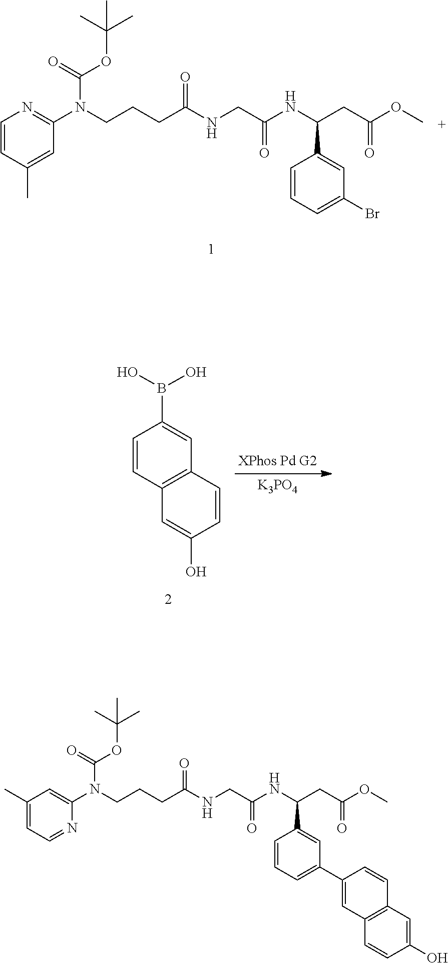

Compound 1 (150 mg, 0.253 mmol, 1.0 equiv.), compound 2 (71.5 mg, 0.380 mmol, 1.5 equiv.), XPhos Pd G2 (4 mg, 0.0051 mmol, 0.02 equiv.), and K₃PO₄ (107 mg, 0.507 mmol, 2.0 equiv.) were mixed in a round-bottom flask. The flask was sealed with a screw-cap septum, and then evacuated and backfilled with nitrogen (this process was repeated a total of 3 times). Then, THF (5 mL) and water (1 mL) were added via syringe. The mixture was bubbled with nitrogen for 10 min and the reaction was kept at 40° C. for 2 hours. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 2-3% methanol in DCM. LC-MS: calculated [M+H]+ 655.31, found 655.78.

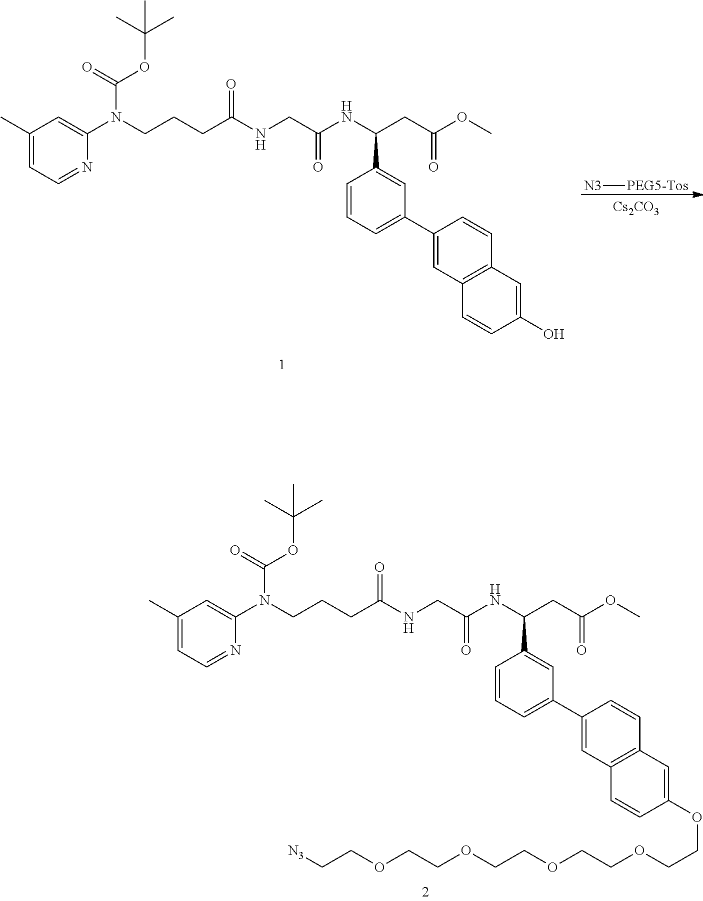

To a solution of compound 1 (104 mg, 0.158 mmol, 1 equiv.) and azido-PEG5-OTs (132 mg, 0.317 mmol, 2 equiv.) in anhydrous DMF (2 mL) was added Cs₂CO₃ (103 mg, 0.317 mmol, 2 equiv.) at room temperature. The reaction mixture was stirred for 3 hours at 60° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 900.44, found 901.01.

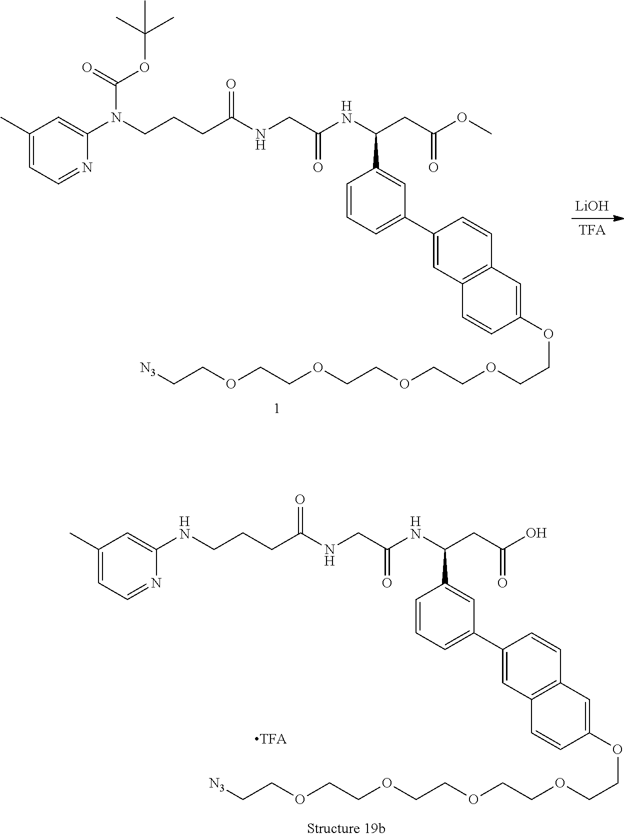 [SMILES: COC(=O)C[C@H](NC(=O)CNC(=O)CCCN(C(=O)OC(C)(C)C)c1cc(C)ccn1)c1cccc(-c2ccc3cc(OCCOCCOCCOCCOCCN=[N+]=[N-])ccc3c2)c1 ; "LiOH" ; Cc1ccnc(NCCCC(=O)NCC(=O)N[C@@H](CC(=O)O)c2cccc(-c3ccc4cc(OCCOCCOCCOCCOCCN=[N+]=[N-])ccc4c3)c2)c1 ; "TFA"]

To a solution of compound 1 (125 mg, 0.138 mmol, 1.0 equiv.) in THE (2 mL) and water (2 mL) was added lithium hydroxide (10 mg, 0.416 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator and the product was separated by CombiFlash® using silica gel as the stationary phase and eluted with 12% methanol in DCM. LC-MS: calculated [M+H]+ 786.37, found 786.86.

### Synthesis of Structure 20b ((S)-3-(3-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

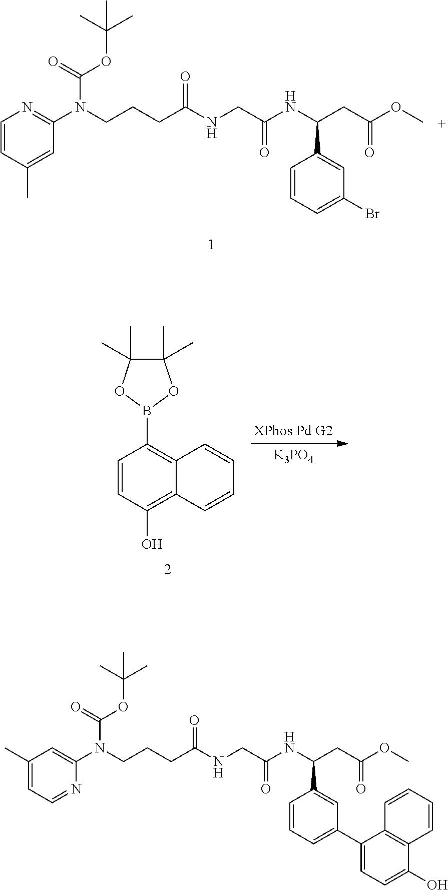

Compound 1 (150 mg, 0.253 mmol, 1.0 equiv.), compound 2 (102 mg, 0.380 mmol, 1.5 equiv.), XPhos Pd G2 (4 mg, 0.0051 mmol, 0.02 equiv.), and K₃PO₄ (107 mg, 0.507 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 10 min and the reaction was kept at 40° C. for 2 hours. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 2-3% methanol in DCM. LC-MS: calculated [M+H]+ 655.31, found 655.78.

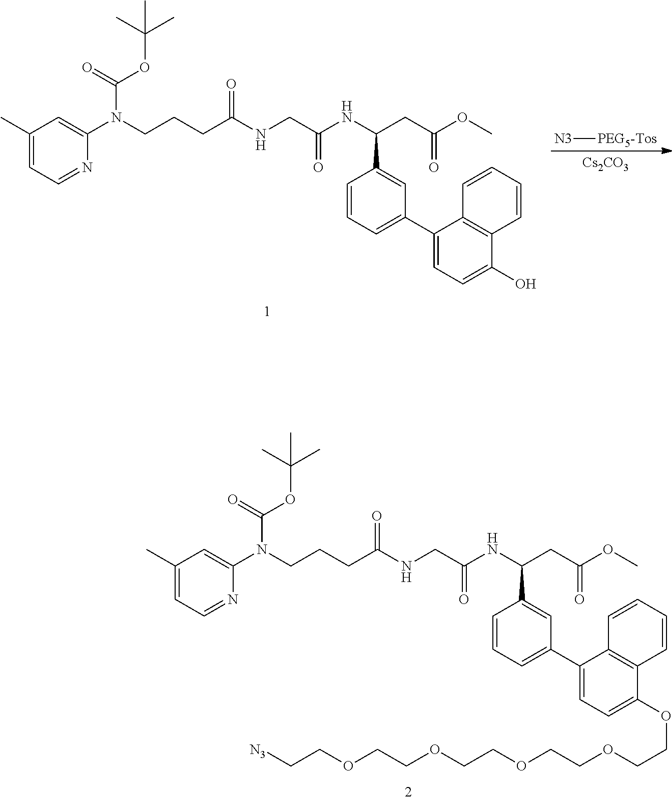

To a solution of compound 1 (160 mg, 0.244 mmol, 1 equiv.) and azido-PEG₅-OTs (204 mg, 0.488 mmol, 2 equiv.) in anhydrous DMF (2 mL) was added Cs₂CO₃ (159 mg, 0.488 mmol, 2 equiv.) at room temperature. The reaction mixture was stirred for 3 hours at 60° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 900.44, found 901.01.

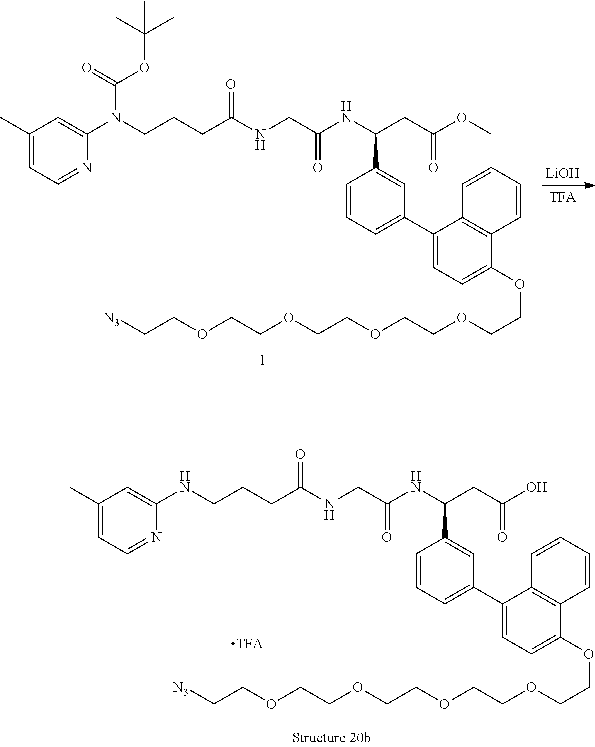

To a solution of compound 1 (125 mg, 0.138 mmol, 1.0 equiv.) in THE (2 mL) and water (2 mL) was added lithium hydroxide (10 mg, 0.416 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator and the product was separated by CombiFlash® using silica gel as the stationary phase and eluted with 8-12% methanol in DCM. LC-MS: calculated [M+H]+ 786.37, found 786.86.

### Synthesis of Structure 22b ((S)-3-(4-(4-((14-azido-3,6,9,12-tetraoxatetradecyl)oxy)naphthalen-1-yl)phenyl)-3-((S)-2-(4-((4-methylpyridin-2-yl)amino)butanamido)propanamido)propanoic acid)

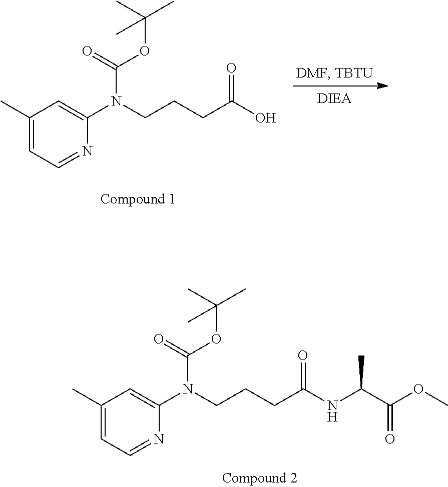

To a solution of compound 1 (250 mg, 0.85 mmol), L-alanine methyl ester hydrochloride salt (130 mg, 0.93 mmol), and TBTU (327 mg, 1.02 mmol) in DMF (2 mL) was added DIPEA (329 mg, 444 μL, 2.55 mmol) at 0° C. The reaction mixture was warmed to room temperature and stirred for 1 hour. The reaction was quenched with sat. NH₄Cl (aq) solution (0.75 mL) and deionized water (1 mL) then extracted with ethyl acetate (3 mL). The aqueous layer was further extracted with ethyl acetate (2×3 mL). The combined organic phase was washed with sat. NaHCO₃(aq) solution (2 mL). The organic layer was dried over Na₂SO₄, filtered, and concentrated. The crude mixture was separated by CombiFlash® using silica gel as the stationary phase with 0-5% methanol in DCM. Yield of compound 2: 294 mg (91%). [M+H] calculated for C₁₉H₂₉N₃O₅: 380.46, found: 380.33.

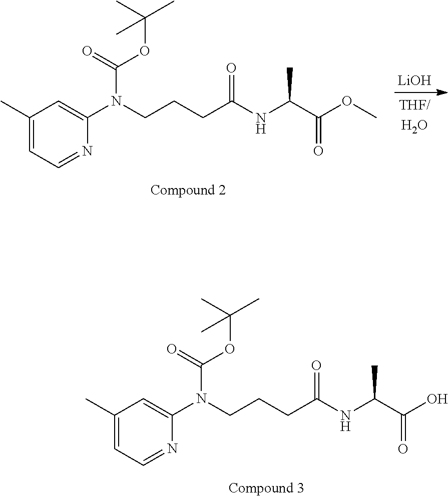

To a solution of compound 2 (294 mg, 0.77 mmol) in THE (4.5 mL) and deionized water (3 mL) at 0° C. was added a solution of lithium hydroxide (56 mg, 2.32 mmol) in deionized water (1 mL). The reaction was warmed to room temperature and stirred for 40 minutes. The reaction mixture was acidified to pH=3 with 6 M HCl(aq). The aqueous phase was extracted with ethyl acetate (3×10 mL). The combined organic phase was dried over Na₂SO₄, filtered, and concentrated. Compound 3 was used without further purification. Yield of compound 3: 267 mg (94%). [M+H] calculated for C₁₈H₂₇N₃O₅: 366.43, found: 366.19.

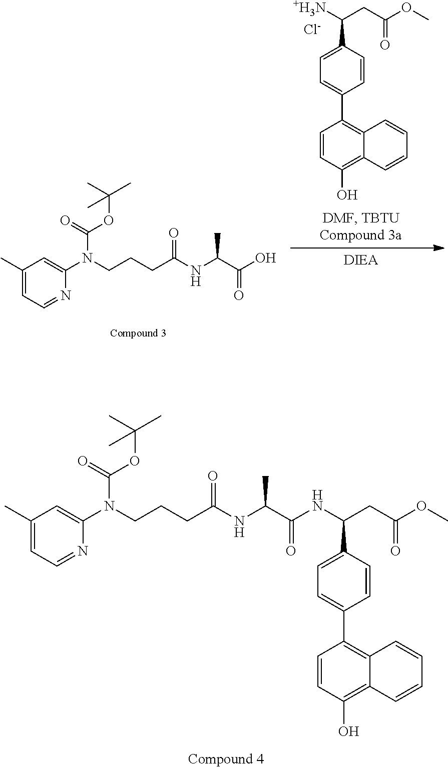

To a solution of compound 3 (267 mg, 0.73 mmol), compound 3a (288 mg, 0.80 mmol), and TBTU (282 mg, 0.88 mmol) in DMF (3 mL) was added DIPEA (283 mg, 382 μL, 2.19 mmol) at 0° C. The reaction mixture was warmed to room temperature and stirred for 1 hour. The reaction mixture was quenched with sat. NH₄Cl (aq) solution (1.5 mL) and deionized water (1.5 mL) then extracted with ethyl acetate (12 mL). The aqueous layer was further extracted with ethyl acetate (2×12 mL). The combined organic phase was washed with half sat. NH₄C₁(aq) solution (10 mL), half sat. NaHCO₃(aq) solution (10 mL), and sat. NaCl (aq) solution (10 mL). The organic layer was dried over Na₂SO₄, filtered, and concentrated. The crude mixture was separated by CombiFlash® using silica gel as the stationary phase with 0-5% methanol in DCM. Yield of compound 4: 342 mg (70%). [M+H] calculated for C₃₈H₄₄N₄O₇: 669.79, found: 669.74.

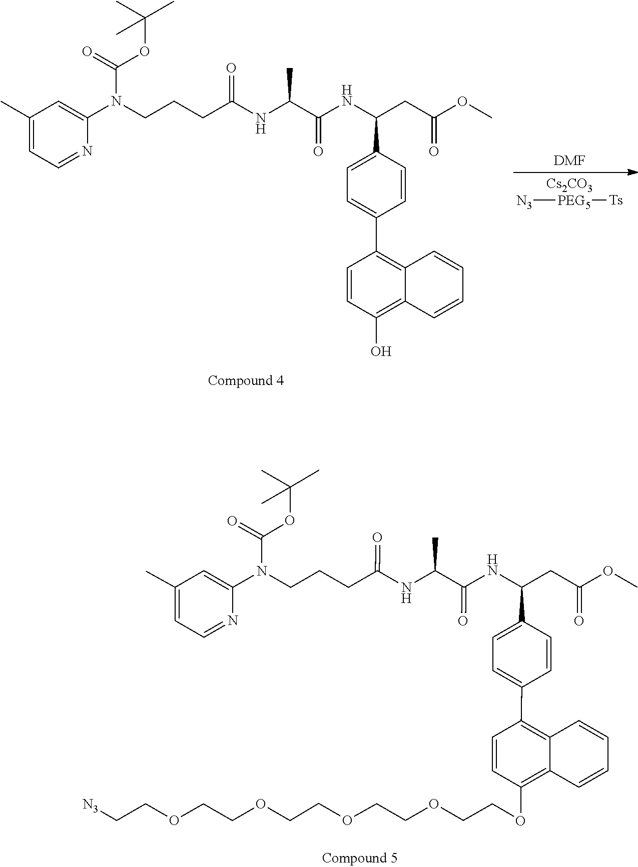

To a solution of compound 4 (150 mg, 0.22 mmol) and azido-PEG₅-OTs (187 mg, 0.49 mmol) in anhydrous DMF (1.2 mL) was added Cs₂CO₃ (146 mg, 0.49 mmol). The reaction mixture was stirred at 60° C. for 3 hours. The reaction mixture was quenched with sat. NaHCO₃(aq) solution (10 mL) and deionized water (5 mL) then extracted with ethyl acetate (7.5 mL). The aqueous layer was further extracted with ethyl acetate (2×7.5 mL). The combined organic phase was dried over Na₂SO₄, filtered, and concentrated. The crude mixture was separated by CombiFlash® using silica gel as the stationary phase with 0-4% methanol in DCM. Yield of compound 5: 142 mg (69%). [M+H] calculated for C₄₈H₆₃N₇O₁₁:

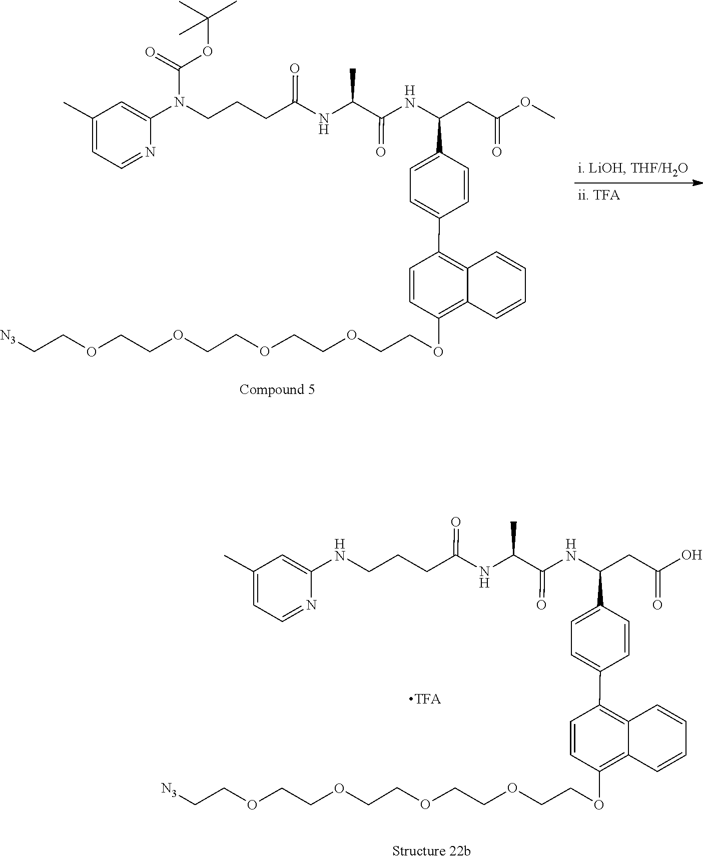 [SMILES: COC(=O)C[C@H](NC(=O)[C@H](C)NC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "i. LiOH, THF/H2O" ; "ii. TFA" ; Cc1ccnc(NCCCC(=O)N[C@@H](C)C(=O)N[C@@H](CC(=O)O)c2ccc(-c3ccc(OCCOCCOCCOCCOCCN=[N+]=[N-])c4ccccc34)cc2)c1]

To a solution of compound 5 (142 mg, 0.16 mmol) in THE (2 mL) and deionized water (1.5 mL) at 0° C. was added a solution of lithium hydroxide (11 mg, 0.47 mmol) in deionized water (0.5 mL). The reaction was warmed to room temperature and stirred for 1 hour. The reaction mixture was acidified to pH=3 with 6 M HCl (aq). The aqueous phase was extracted with ethyl acetate (3×8 mL). The combined organic phase was dried over Na₂SO₄, filtered, and concentrated. To the crude residue was added TFA (2.0 mL) and water (100 μL). The reaction mixture was stirred for 1.5 hours at room temperature. The solvent was removed under reduced pressure, and the residue was coevaporated with acetonitrile:toluene [1:1] (2×20 mL). The crude mixture was separated by CombiFlash® using silica gel as the stationary phase with 0-13% methanol in DCM. Yield of Structure 22b: 100 mg (80%). [M+H] calculated for C₄₂H₅₃N₇O₉: 800.92, found: 800.81.

### Synthesis of Structure 23b ((S)-3-(4-(4-((14-azido-3,6,9,12-tetraoxatetradecyl)oxy)naphthalen-1-yl)phenyl)-3-((S)-3-methyl-2-(4-((4-methylpyridin-2-yl)amino)butanamido)butanamido)propanoic acid)

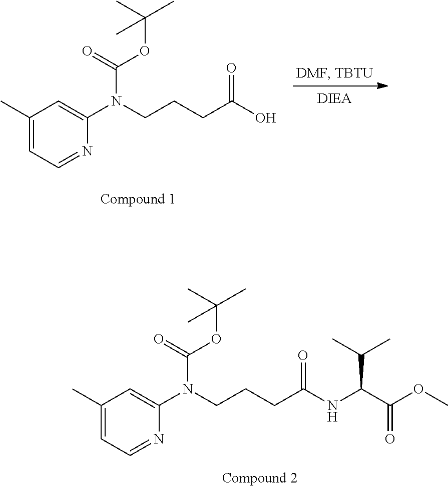

To a solution of compound 1 (250 mg, 0.85 mmol), L-valine methyl ester hydrochloride salt (157 mg, 0.93 mmol), and TBTU (327 mg, 1.02 mmol) in DMF (2 mL) was added DIPEA (329 mg, 444 μL, 2.55 mmol) at 0° C. The reaction mixture was warmed to room temperature and stirred for 1 hour. The reaction was quenched with sat. NH₄Cl (aq) solution (0.75 mL) and deionized water (1 mL) then extracted with ethyl acetate (3 mL). The aqueous layer was further extracted with ethyl acetate (2×3 mL). The combined organic phase was washed with sat. NaHCO₃(aq) solution (2 mL). The organic layer was dried over Na₂SO₄, filtered, and concentrated. The crude mixture was separated by CombiFlash® using silica gel as the stationary phase with 0-5% methanol in DCM. Yield of compound 2: 297 mg (86%). [M+H] calculated for C₂₁H₃₃N₃O₅: 408.51, found: 407.87.

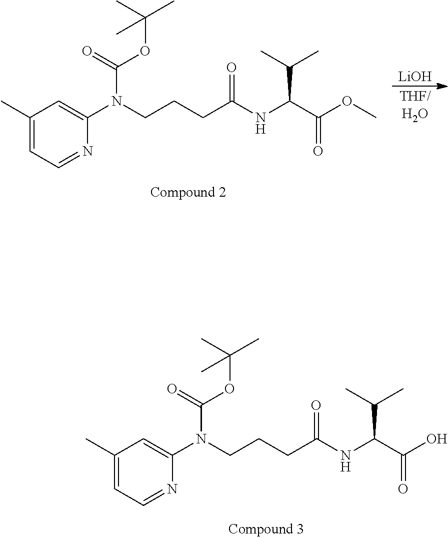

To a solution of compound 2 (297 mg, 0.73 mmol) in THE (4.5 mL) and deionized water (3 mL) at 0° C. was added a solution of lithium hydroxide (52 mg, 2.19 mmol) in deionized water (1 mL). The reaction was warmed to room temperature and stirred for 40 minutes. The reaction mixture was acidified to pH=3 with 6 M HCl(aq). The aqueous phase was extracted with ethyl acetate (3×10 mL). The combined organic phase was dried over Na₂SO₄, filtered, and concentrated. Compound 3 was used without further purification assuming 100% yield. [M+H] calculated for C₂₀H₃₁N₃O₅: 394.49, found: 393.83.

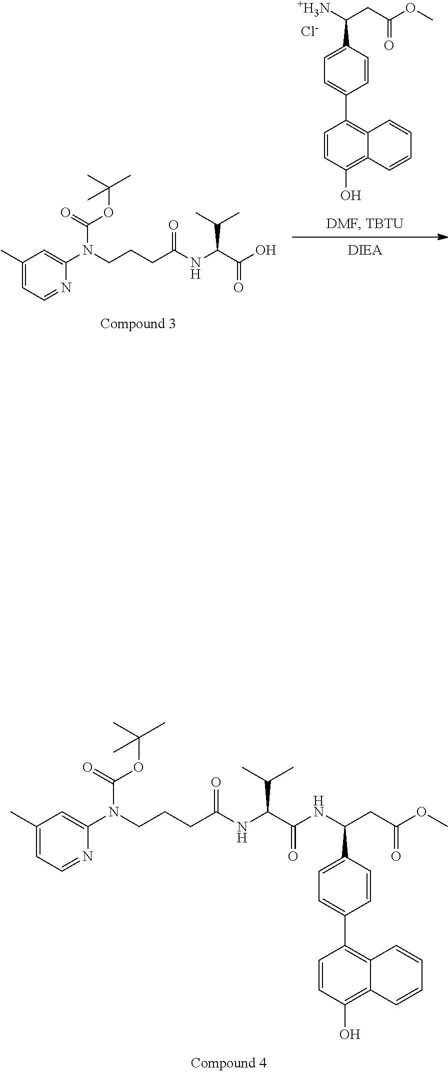

To a solution of compound 3 (287 mg, 0.73 mmol), compound 3a (287 mg, 0.80 mmol), and TBTU (281 mg, 0.88 mmol) in DMF (3 mL) was added DIPEA (283 mg, 382 μL, 2.19 mmol) at 0° C. The reaction mixture was warmed to room temperature and stirred for 1 hour. The reaction mixture was quenched with sat. NH₄Cl (aq) solution (2.5 mL) and deionized water (2.5 mL) then extracted with ethyl acetate (12 mL). The aqueous layer was further extracted with ethyl acetate (2×12 mL). The combined organic phase was washed with half sat. NH₄C₁(aq) solution (10 mL), half sat. NaHCO₃(aq) solution (10 mL), and sat. NaCl (aq) solution (10 mL). The organic layer was dried over Na₂SO₄, filtered, and concentrated. The crude mixture was separated by CombiFlash® using silica gel as the stationary phase with 0-5% methanol in DCM. Yield of compound 4: 374 mg (74%). [M+H] calculated for C₄₀H₄₈N₄O₇: 697.84, found: 697.46.

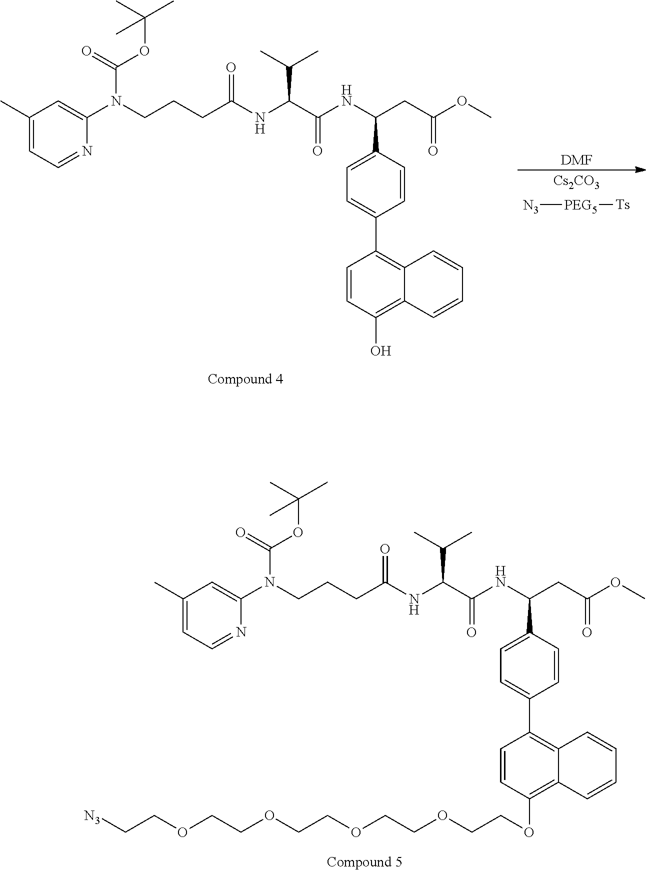

To a solution of compound 4 (150 mg, 0.215 mmol) and azido-PEG₅-OTs (180 mg, 0.43 mmol) in anhydrous DMF (1.2 mL) was added Cs₂CO₃ (140 mg, 0.43 mmol). The reaction mixture was stirred at 60° C. for 3 hours. The reaction mixture was quenched with sat. NaHCO₃(aq) solution (10 mL) and deionized water (5 mL) then extracted with ethyl acetate (7.5 mL). The aqueous layer was further extracted with ethyl acetate (2×7.5 mL). The combined organic phase was dried over Na₂SO₄, filtered, and concentrated. The crude mixture was separated by CombiFlash® using silica gel as the stationary phase with 0-4% methanol in DCM. Yield of compound 5: 134 mg (66%). [M+H] calculated for C₅₀H₆₇N₇O₁₁: 943.12, found: 942.96.

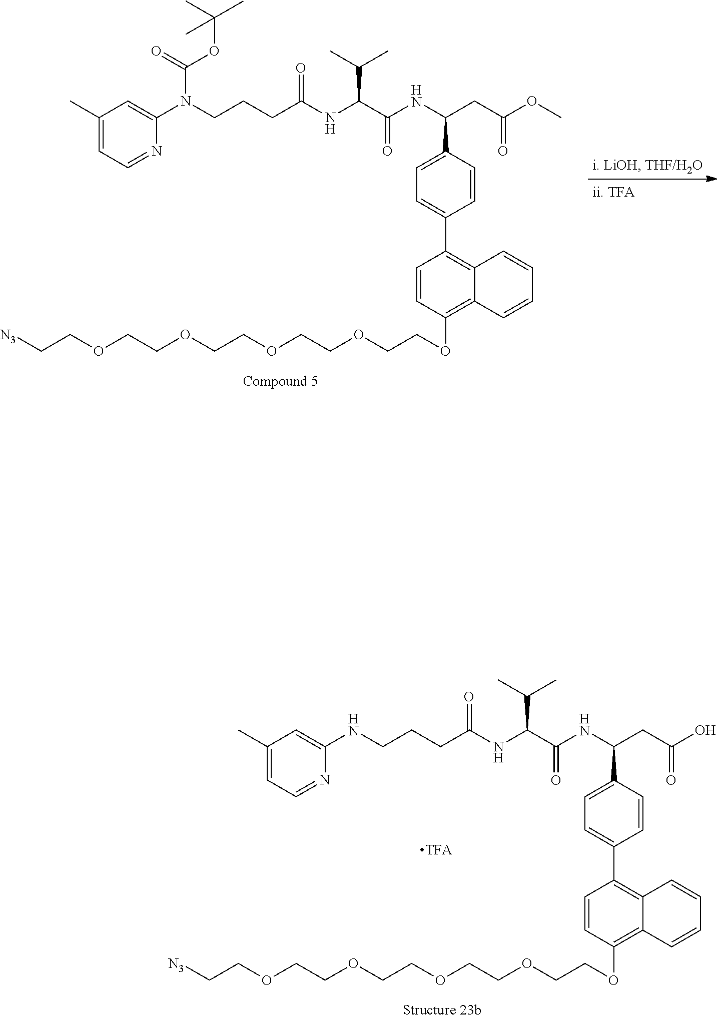 [SMILES: COC(=O)C[C@H](NC(=O)[C@@H](NC(=O)CCCN(C(=O)OC(C)(C)C)c1cc(C)ccn1)C(C)C)c1ccc(-c2ccc(OCCOCCOCCOCCOCCN=[N+]=[N-])c3ccccc23)cc1 ; Cc1ccnc(NCCCC(=O)N[C@H](C(=O)N[C@@H](CC(=O)O)c2ccc(-c3ccc(OCCOCCOCCOCCOCCN=[N+]=[N-])c4ccccc34)cc2)C(C)C)c1]

To a solution of compound 5 (134 mg, 0.14 mmol) in THE (2 mL) and deionized water (1.5 mL) at 0° C. was added a solution of lithium hydroxide (10 mg, 0.43 mmol) in deionized water (0.5 mL). The reaction was warmed to room temperature and stirred for 1 hour. The reaction mixture was acidified to pH=3 with 6 M HCl (aq). The aqueous phase was extracted with ethyl acetate (3×8 mL). The combined organic phase was dried over Na₂SO₄, filtered, and concentrated. To the crude residue was added TFA (1.9 mL) and water (95 μL). The reaction mixture was stirred for 1.5 hours at room temperature. The solvent was removed under reduced pressure, and the residue was coevaporated with acetonitrile:toluene [1:1] (2×20 mL). The crude mixture was separated by CombiFlash® using silica gel as the stationary phase with 0-10% methanol in DCM. Yield of Structure 23b: 36 mg (30.5%). [M+H] calculated for C₄₄H₅₇N₇O₉: 828.97, found 828.90.

### Synthesis of Structure 24b ((S)-3-(4-(4-((14-azido-3,6,9,12-tetraoxatetradecyl)oxy)naphthalen-1-yl)phenyl)-3-((S)-2-(4-((4-methylpyridin-2-yl)amino)butanamido)-3 phenylpropanamido)propanoic acid)

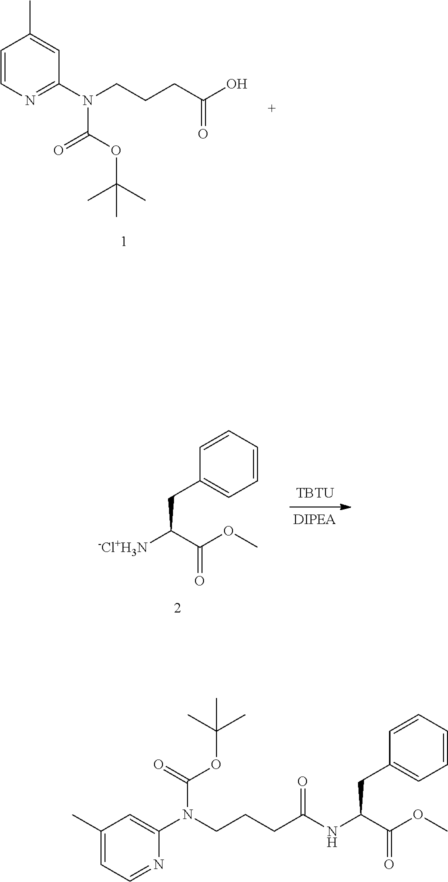

To a solution of compound 1 (200 mg, 0.679 mmol, 1 equiv.), compound 2 (161 mg, 0.747 mmol, 1.2 equiv.), and TBTU (261 mg, 0.815 mmol, 1.2 equiv.) in anhydrous DMF (4 mL) was added diisopropylethylamine (0.355 mL, 2.038 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 2-3% methanol in DCM. LC-MS: calculated [M+H]+ 456.24, found 456.12.

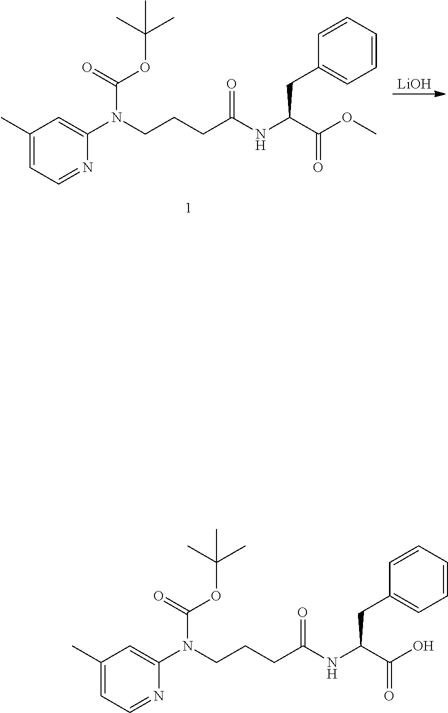

To a solution of compound 1 (300 mg, 0.658 mmol, 1 equiv.) in THE (5 mL) and H₂O (5 mL) was added lithium hydroxide (47 mg, 1.975 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 442.23, found 442.08.

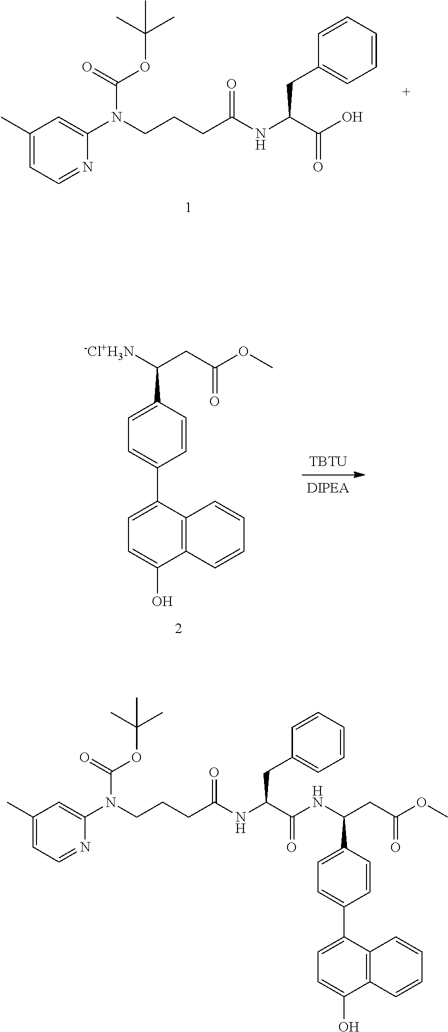

To a solution of compound 1 (290 mg, 0.656 mmol, 1 equiv.), compound 2 (258 mg, 0.722 mmol, 1.1 equiv.), and TBTU (253 mg, 0.788 mmol, 1.2 equiv.) in anhydrous DMF (5 mL) was added diisopropylethylamine (0.343 mL, 1.970 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 3-4% methanol in DCM. LC-MS: calculated [M+H]+ 745.35, found 745.63.

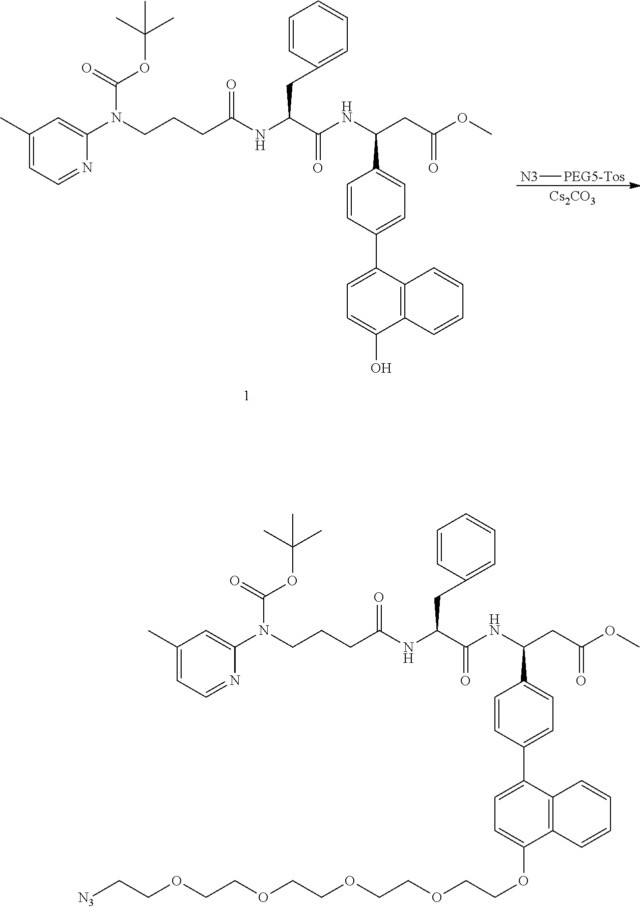

To a solution of compound 1 (113 mg, 0.151 mmol, 1 equiv.) and azido-PEG₅-OTs (126 mg, 0.303 mmol, 2 equiv.) in anhydrous DMF (2 mL) was added Cs₂CO₃ (99 mg, 0.303 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 990.49, found 990.87.

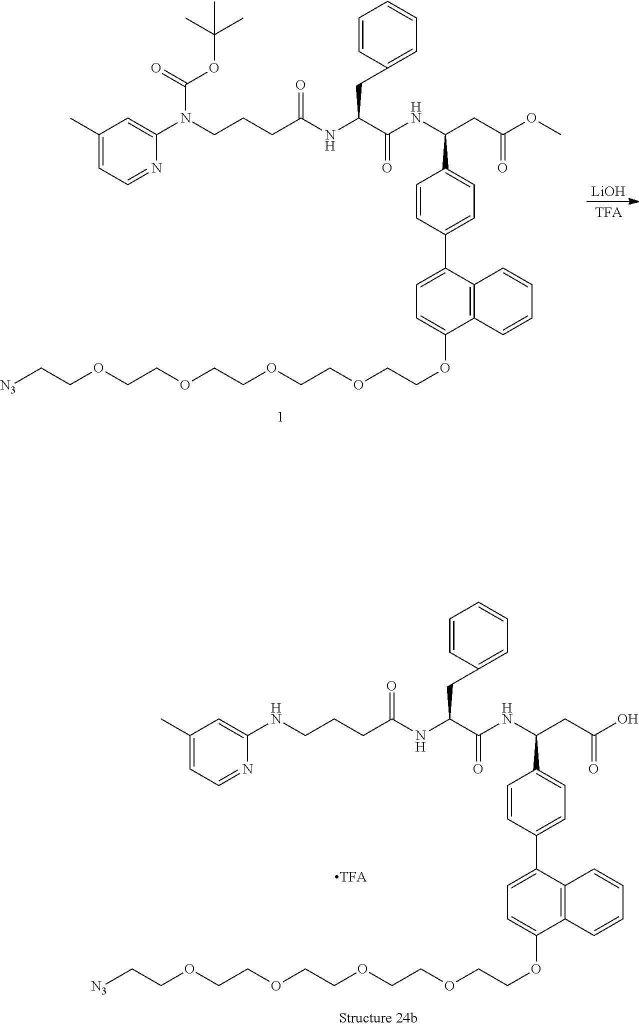

To a solution of compound 1 (140 mg, 0.141 mmol, 1.0 equiv.) in THF (2 mL) and water (2 mL) was added lithium hydroxide (10 mg, 0.424 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator and the product was separated by CombiFlash® using silica gel as the stationary phase and eluted with 6-10% methanol in DCM. LC-MS: calculated [M+H]+ 876.42, found 876.88.

### Synthesis of Structure 25b ((S)-3-(4-(4-((14-azido-3,6,9,12-tetraoxatetradecyl)oxy)naphthalen-1-yl)phenyl)-3-((S)-3-(benzyloxy)-2-(4-((4-methylpyridin-2-yl)amino)butanamido)propanamido)propanoic acid)

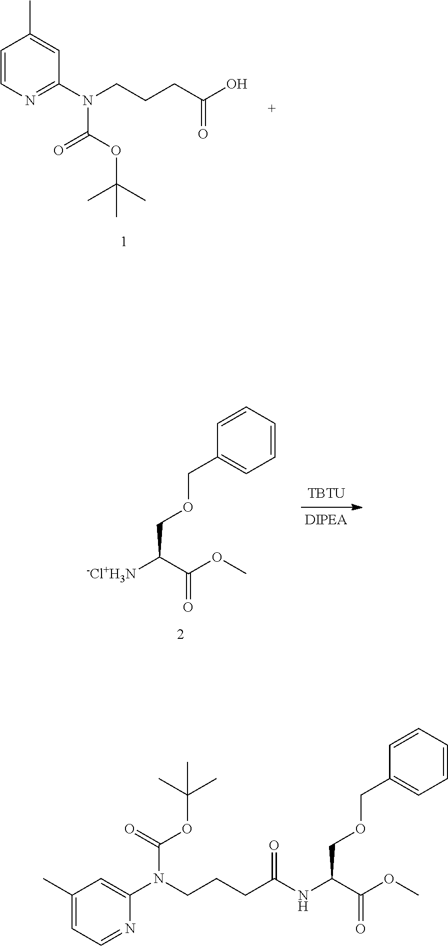

To a solution of compound 1 (100 mg, 0.339 mmol, 1 equiv.), compound 2 (92 mg, 0.373 mmol, 1.1 equiv.), and TBTU (131 mg, 0.407 mmol, 1.2 equiv.) in anhydrous DMF (4 mL) was added diisopropylethylamine (0.178 mL, 1.019 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 2-4% methanol in DCM. LC-MS: calculated [M+H]+ 486.25, found 486.37.

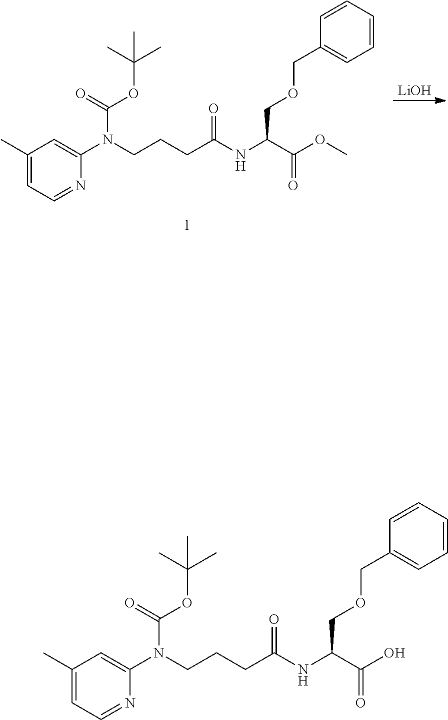 [SMILES: COC(=O)[C@H](COCc1ccccc1)NC(=O)CCCN(C(=O)OC(C)(C)C)c1cc(C)ccn1 ; "LiOH" ; Cc1ccnc(N(CCCC(=O)N[C@@H](COCc2ccccc2)C(=O)O)C(=O)OC(C)(C)C)c1]

To a solution of compound 1 (160 mg, 0.329 mmol, 1 equiv.) in THF (5 mL) and H₂O (5 mL) was added lithium hydroxide (23 mg, 0.988 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 472.24, found 472.32.

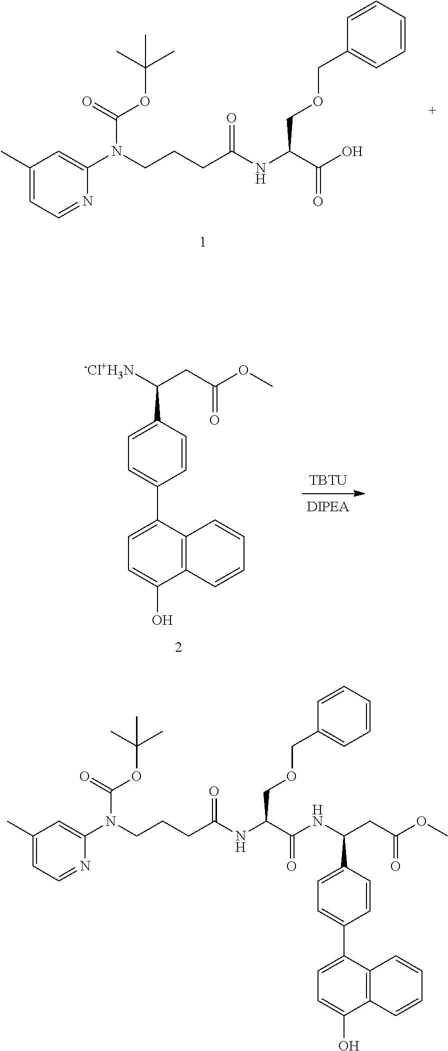

To a solution of compound 1 (1600 mg, 0.339 mmol, 1 equiv.), compound 2 (133 mg, 0.373 mmol, 1.1 equiv.), and TBTU (130 mg, 0.815 mmol, 1.2 equiv.) in anhydrous DMF (3 mL) was added diisopropylethylamine (0.177 mL, 1.018 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 2-3% methanol in DCM. LC-MS: calculated [M+H]+ 775.36, found 775.87.

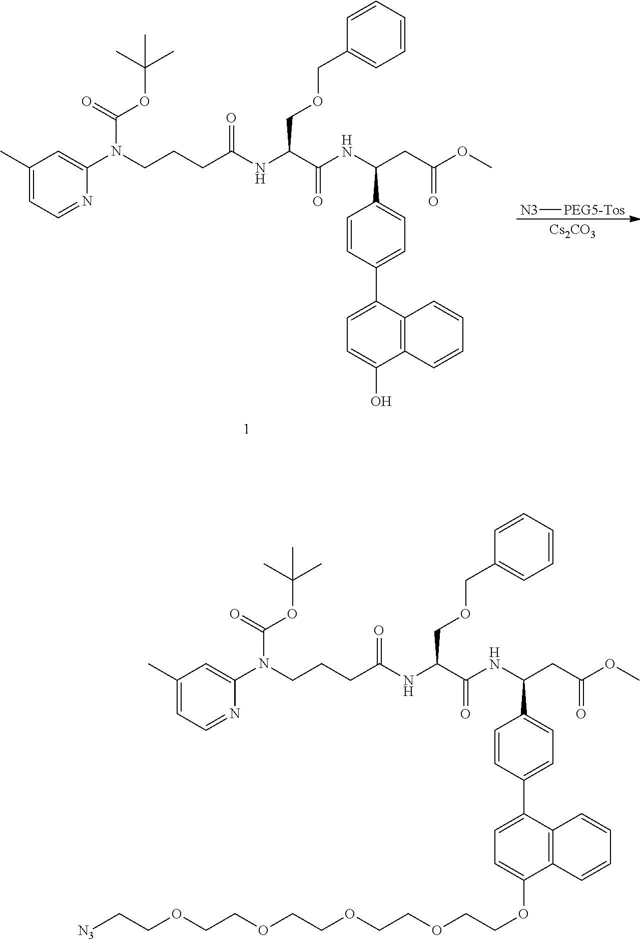

To a solution of compound 1 (140 mg, 0.180 mmol, 1 equiv.) and azido-PEG₅-OTs (150 mg, 0.361 mmol, 2 equiv.) in anhydrous DMF (2 mL) was added Cs₂CO₃ (117 mg, 0.361 mmol, 2 equiv.) at room temperature. The reaction mixture was stirred for 3 hours at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 1020.50, found 1020.88.

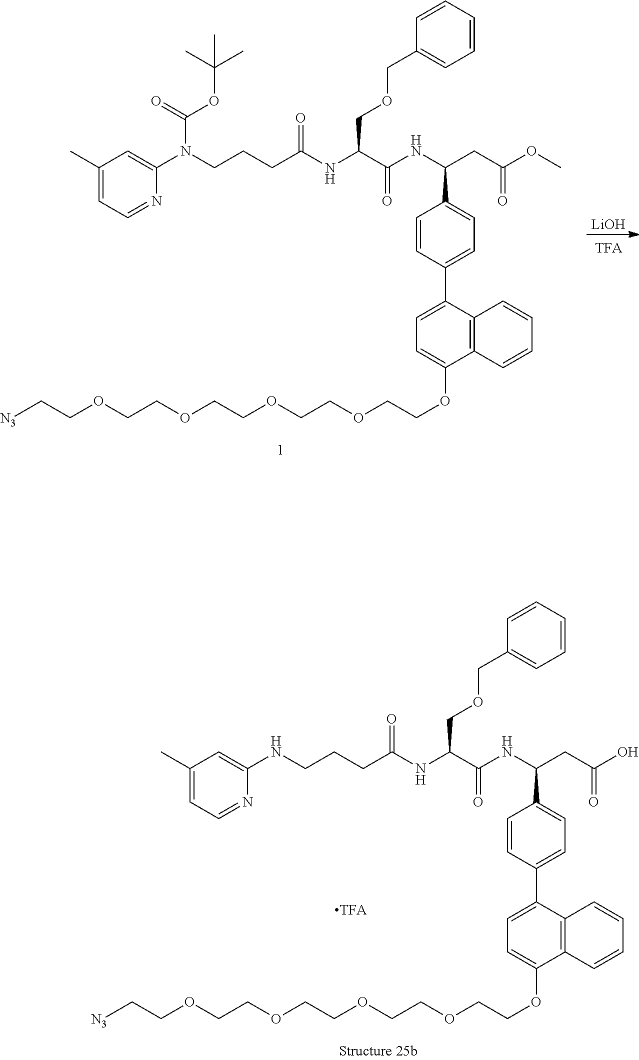

To a solution of compound 1 (170 mg, 0.166 mmol, 1.0 equiv.) in THE (2 mL) and water (2 mL) was added lithium hydroxide (12 mg, 0.499 mmol, 3.0 equiv.) at room temperature. The mixture was stirred at room temperature for another 1 hours. The pH was adjusted to 3.0 by HCl (6N) and the aqueous phase was extracted with EtOAc (3×10 mL). The organic phase was combined, dried over Na₂SO₄, and concentrated. TFA (4 mL) and DCM (2 mL) was added into the residue and the mixture was stirred at room temperature for another 3 hr. The solvent was removed by rotary evaporator and the product was separated by CombiFlash® using silica gel as the stationary phase and eluted with 6-10% methanol in DCM. LC-MS: calculated [M+H]+ 906.43, found 906.95.

### Synthesis of Structure 27b ((S)-3-(3-(4-((14-azido-3,6,9,12-tetraoxatetradecyl)oxy)-3,5-dimethyl-1H-pyrazol-1-yl)phenyl)-3-(2-(4-((4-methylpyridin-2-yl)amino)butanamido)acetamido)propanoic acid)

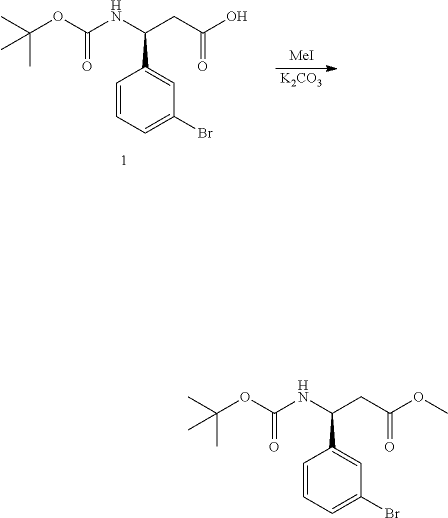

To a solution of compound 1 (3.0 g, 8.71 mmol, 1 equiv.) and potassium carbonate (1.806 g, 13.073 mmol, 1.5 equiv.) in anhydrous DMF (10 mL) was added methyl iodide (1.085 mL, 17.431 mmol, 2.0 equiv.) at room temperature. The reaction mixture was stirred at room temperature for 1 hr. The reaction was then quenched with water (20 mL) and the aqueous phase was extracted with ethyl acetate (3×10 mL). The organic phase was combined, dried over anhydrous Na₂SO₄, and concentrated. The product was separated by CombiFlash® using silica gel as the stationary phase and was eluted with 15% ethyl acetate in hexane. LC-MS: calculated [M+H]+ 358.06, found 358.15.

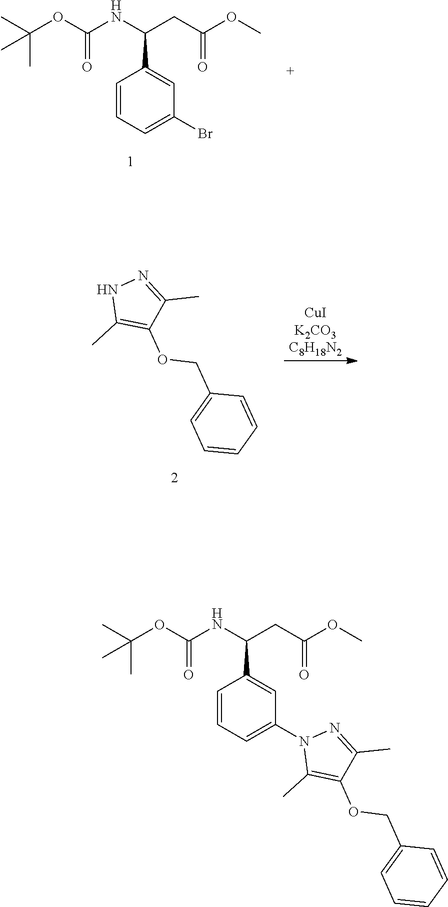 [SMILES: COC(=O)C[C@H](NC(=O)OC(C)(C)C)c1cccc(Br)c1 ; Cc1n[nH]c(C)c1OCc1ccccc1 ; COC(=O)C[C@H](NC(=O)OC(C)(C)C)c1cccc(-n2nc(C)c(OCc3ccccc3)c2C)c1]

The mixture of compound 1 (200 mg, 0.558 mmol, 1 equiv.), compound 2 (169 mg, 0.837 mmol, 1,5 equiv.), copper (I) iodide (106 mg, 0.558 mmol, 1.0 equiv.), potassium carbonate (154 mg, 1.116 mmol, 2.0 equiv.) and trans-N,N′-dimethylcyclohexane-1,2-diamine (88 μL, 0.558 mmol, 1.0 equiv.) in anhydrous DMF (5 mL) was backfilled with nitrogen 3 times. The mixture was stirred at 120° C. for 24 hrs. The mixture was cooled to room temperature and was concentrated. The product was separated by CombiFlash® using silica gel as the stationary phase and was eluted with 30-40% ethyl acetate in hexane. LC-MS: calculated [M+H]+ 480.24, found 480.43.

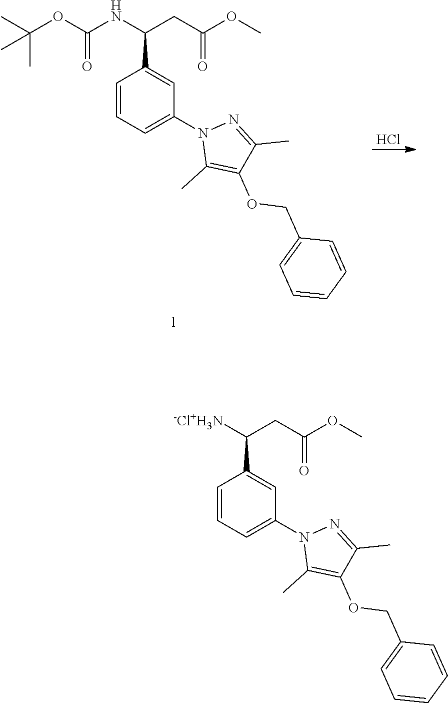

Compound 1 (30 mg, 0.0626 mmol, 1.0 equiv.) was cooled by ice bath. HCl in dioxane (0.313 mL, 1.25 mmol, 20 equiv.) was added into the flask. The reaction was warmed to room temperature and stirred for another 1 hr. The solvent was removed by rotary evaporator and the product was directly used without further purification. LC-MS: calculated [M+H]+ 380.19, found 380.33.

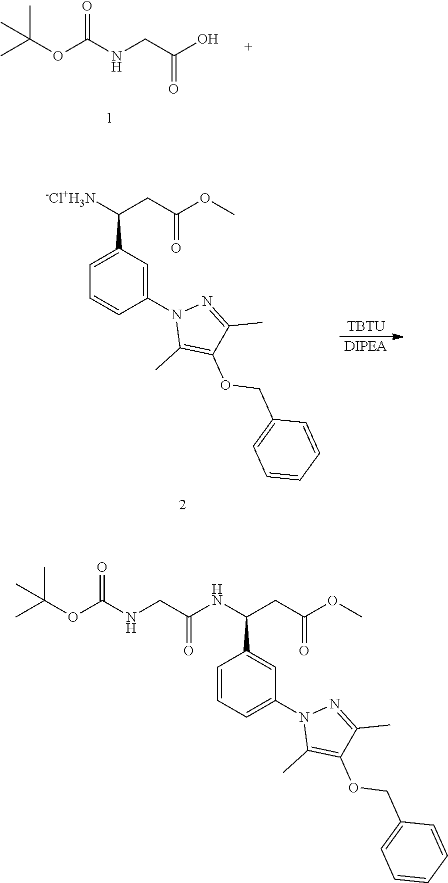

To a solution of compound 1 (10 mg, 0.0571 mmol, 1 equiv.), compound 2 (26 mg, 0.0628 mmol, 1.1 equiv.), and TBTU (22 mg, 0.0685 mmol, 1.2 equiv.) in anhydrous DMF (1 mL) was added diisopropylethylamine (0.030 mL, 0.171 mmol, 3 equiv.) at 0° C. The reaction mixture was warmed to room temperature and stirred for another 1 hr. The reaction was quenched with saturated NaHCO₃ solution (5 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 3-4% methanol in DCM. LC-MS: calculated [M+H]+ 537.26, found 537.41.

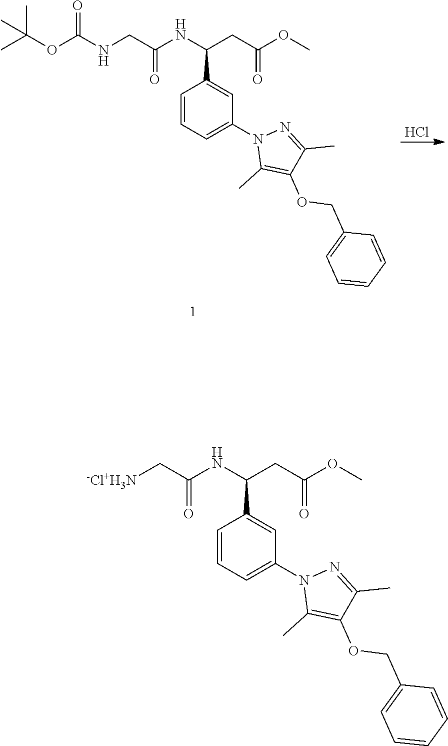

Compound 1 (30 mg, 0.0626 mmol, 1.0 equiv.) was cooled by ice bath. HCl in dioxane (0.313 mL, 1.25 mmol, 20 equiv.) was added into the flask. The reaction was warmed to room temperature and stirred for another 1 hr. The solvent was removed by rotary evaporator and the product was directly used without further purification. LC-MS: calculated [M+H]+ 437.21, found 437.31.

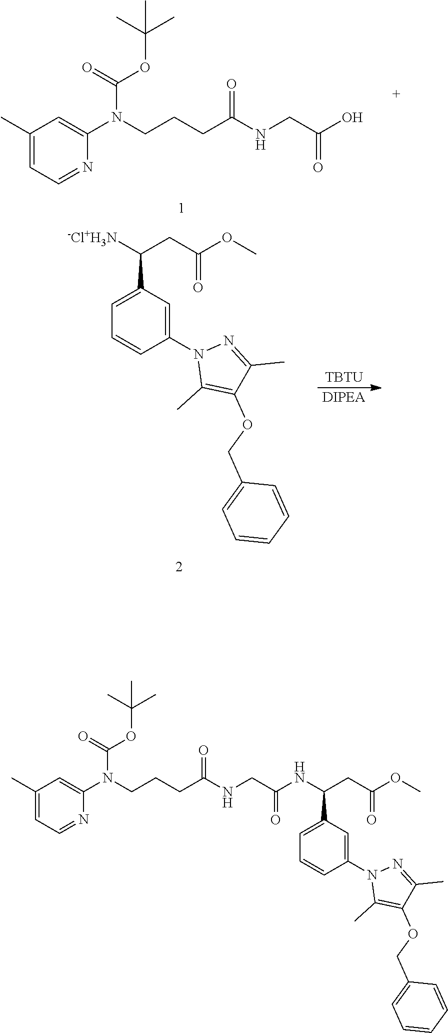

To a solution of compound 1 (20 mg, 0.0569 mmol, 1 equiv.), compound 2 (26 mg, 0.0626 mmol, 1.1 equiv.), and TBTU (22 mg, 0.0683 mmol, 1.2 equiv.) in anhydrous DMF (2 mL) was added diisopropylethylamine (0.03 mL, 0.170 mmol, 3 equiv.) at 0° C. The reaction mixture was warmed to room temperature and stirred for another 1 hr. The reaction was quenched with saturated NaHCO₃ solution (5 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 4-5% methanol in DCM. LC-MS: calculated [M+H]+ 713.36, found 713.85.

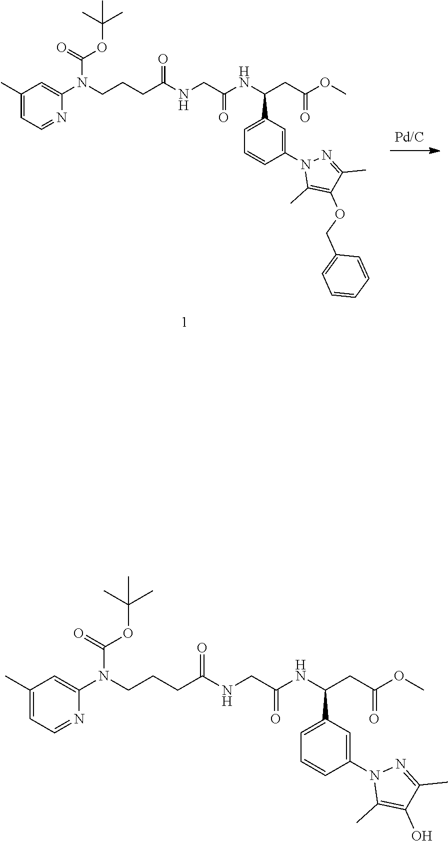

To a solution of compound 1 (0.033 g, 0.0463 mmol, 1 equiv.) in ethyl acetate (10 mL) was added 10% Pd/C (20 mg) at room temperature. The reaction mixture was stirred with hydrogen gas at room temperature for overnight. The catalyst was removed by filtration through Celite® and the product was used directly without further purification. LC-MS: calculated [M+H]+ 623.31, found 623.56.

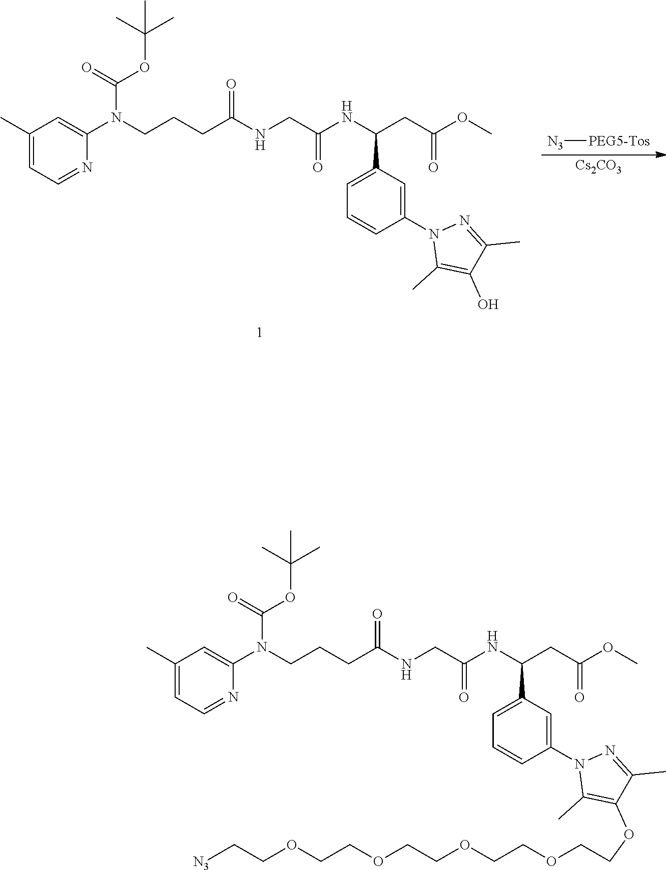

To a solution of compound 1 (16 mg, 0.0257 mmol, 1 equiv.) and azido-PEG5-OTs (22 mg, 0.0514 mmol, 2 equiv.) in anhydrous DMF (2 mL) was added Cs₂CO₃ (17 mg, 0.0514 mmol, 2 equiv.) at room temperature. The reaction mixture was stirred for 3 hrs at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 868.45, found 868.96.

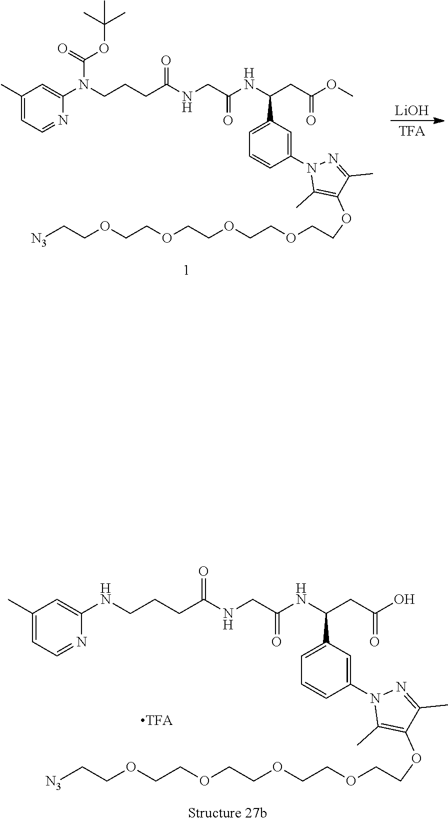

To a solution of compound 1 (5 mg, 0.0058 mmol, 1.0 equiv.) in THE (1 mL) and water (1 mL) was added lithium hydroxide (1 mg, 0.0346 mmol, 6.0 equiv.) at room temperature. The mixture was stirred at room temperature for another 1 hrs. The pH was adjusted to 3.0 by HCl (6N) and the aqueous phase was extracted with EtOAc (3×10 mL). The organic phase was combined, dried over Na₂SO₄, and concentrated. TFA (1 mL) and DCM (1 mL) was added into the residue and the mixture was stirred at room temperature for another 3 hr. The solvent was removed by rotary evaporator. LC-MS: calculated [M+H]+ 754.38, found 755.

### Synthesis of Structure 29b ((S)-3-(4-(3-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

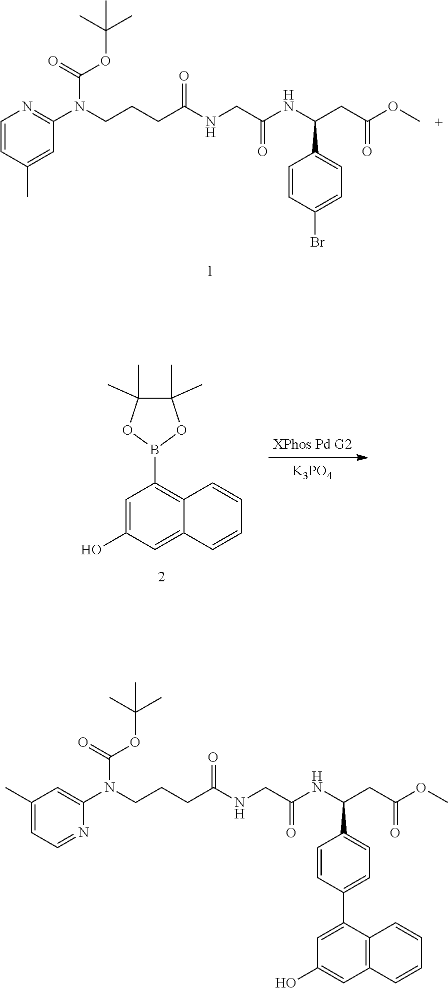

Compound 1 (100 mg, 0.169 mmol, 1.0 equiv.), compound 2 (68 mg, 0.253 mmol, 1.5 equiv.), XPhos Pd G2 (3 mg, 0.0034 mmol, 0.02 equiv.), and K₃PO₄ (72 mg, 0.338 mmol, 2.0 equiv.) were mixed in a round-bottom flask. The flask was sealed with a screw-cap septum, and then evacuated and backfilled with nitrogen (this process was repeated a total of 3 times). Then, THE (5 mL) and water (1 mL) were added via syringe. The mixture was bubbled with nitrogen for 10 min and the reaction was kept at 40° C. for 2 hrs. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 4% methanol in DCM. LC-MS: calculated [M+H]+ 655.31, found 656.

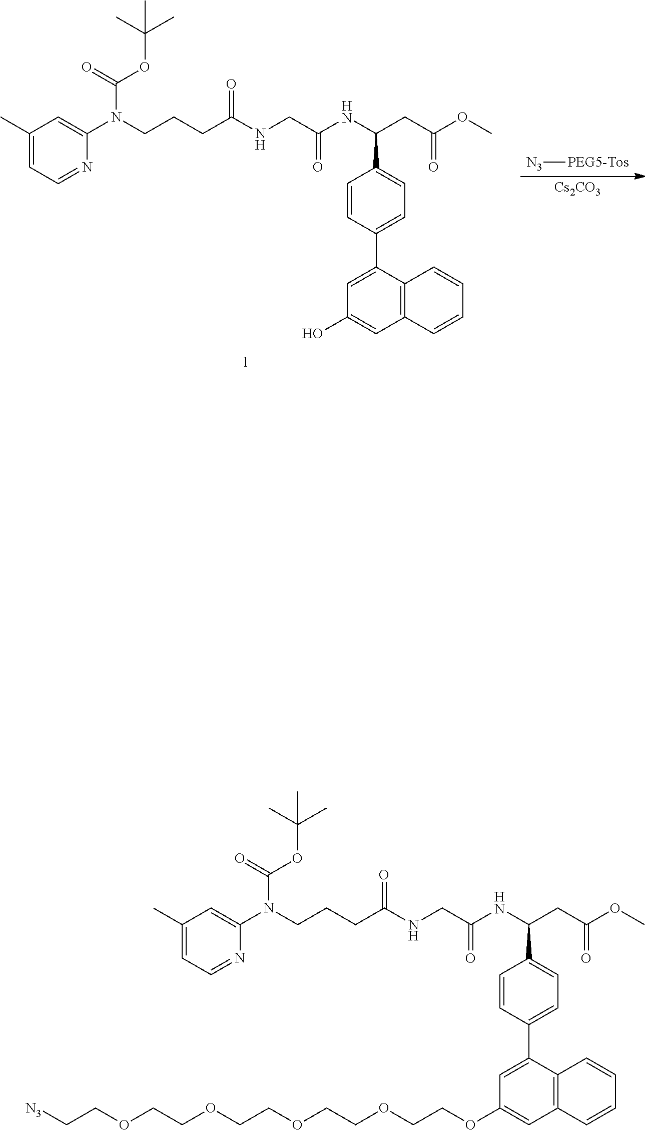

To a solution of compound 1 (100 mg, 0.152 mmol, 1 equiv.) and azido-PEG5-OTs (127 mg, 0.305 mmol, 2 equiv.) in anhydrous DMF (2 mL) was added Cs₂CO₃ (100 mg, 0.305 mmol, 2 equiv.) at room temperature. The reaction mixture was stirred for 3 hrs at 40° C. The reaction was quenched by saturated NaHCO₃ solution (10 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 3-4% methanol in DCM. LC-MS: calculated [M+H]+ 900.44, found 901.

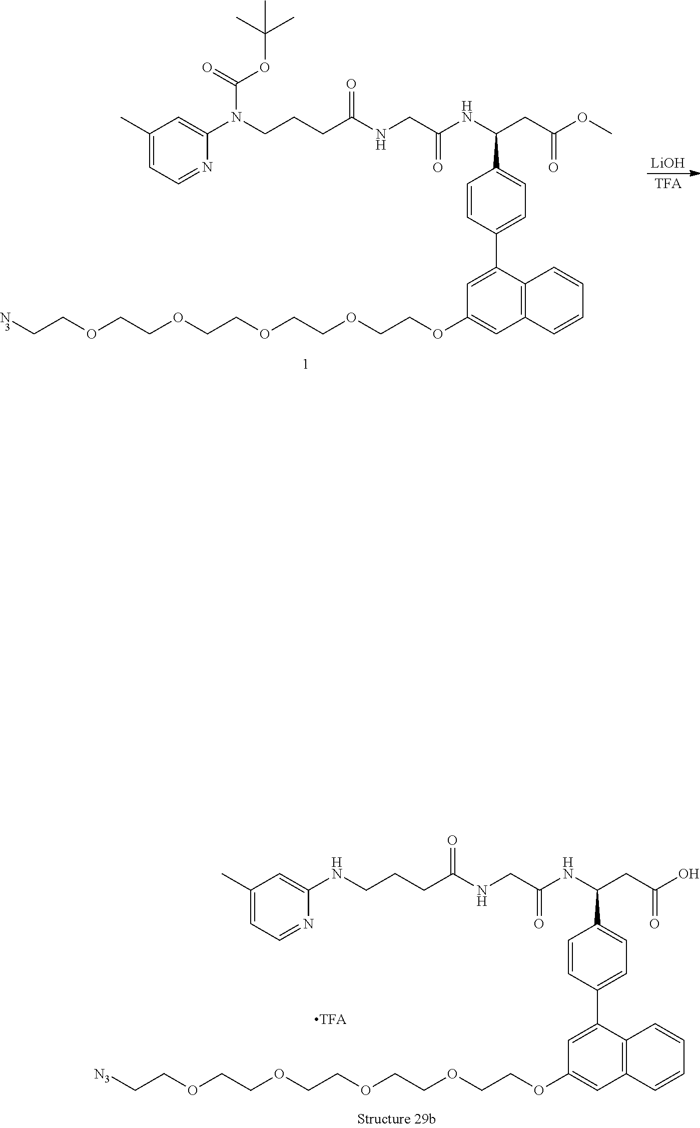

To a solution of compound 1 (125 mg, 0.138 mmol, 1.0 equiv.) in THE (1 mL) and water (1 mL) was added lithium hydroxide (10 mg, 0.416 mmol, 3.0 equiv.) at room temperature. The mixture was stirred at room temperature for another 1 hrs. The pH was adjusted to 3.0 by HCl (6N) and the aqueous phase was extracted with EtOAc (3×10 mL). The organic phase was combined, dried over Na₂SO₄, and concentrated. TFA (3 mL) and DCM (2 mL) was added into the residue and the mixture was stirred at room temperature for another 3 hr. The solvent was removed by rotary evaporator. The product was used directly without further purification. LC-MS: calculated [M+H]+ 786.37, found 787.

### Synthesis of Structure 30b ((S)—N-(1-azido-21-(4-(naphthalen-1-yl)phenyl)-19,23-dioxo-3,6,9,12,15 pentaoxa-18,22-diazatetracosan-24-yl)-4-((4-methylpyridin-2-yl)amino)butanamide)

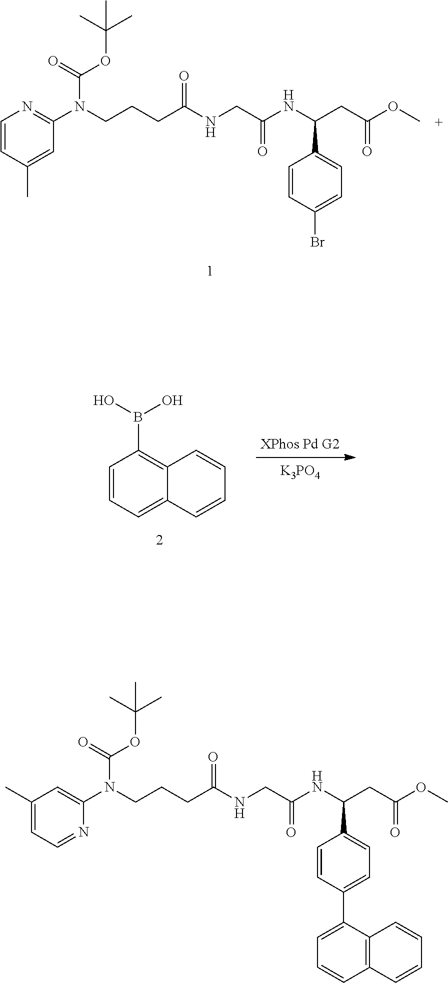

Compound 1 (100 mg, 0.169 mmol, 1.0 equiv.), compound 2 (43 mg, 0.253 mmol, 1.5 equiv.), XPhos Pd G2 (3 mg, 0.0034 mmol, 0.02 equiv.), and K₃PO₄ (72 mg, 0.338 mmol, 2.0 equiv.) were mixed in a round-bottom flask. The flask was sealed with a screw-cap septum, and then evacuated and backfilled with nitrogen (this process was repeated a total of 3 times). Then, THF (5 mL) and water (1 mL) were added via syringe. The mixture was bubbled with nitrogen for 10 min and the reaction was kept at 40° C. for 2 hrs. The reaction was quenched with water (10 mL), and the aqueous phase was extracted with ethyl acetate (3×10 mL). The organic phase was combined, dried over Na₂SO₄, and concentrated. The compound was separated by CombiFlash® using silica gel as the stationary phase and was eluted with 3-4% methanol in DCM. LC-MS: calculated [M+H]+ 639.31, found 640.

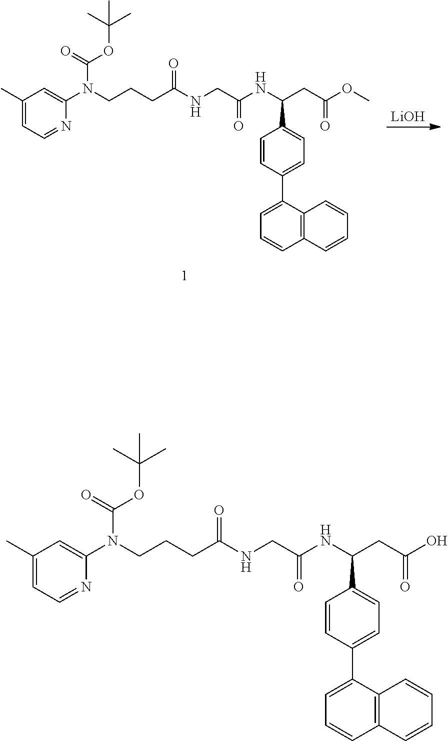

To a solution of compound 1 (90 mg, 0.140 mmol, 1 equiv.) in THF (5 mL) and H₂O (5 mL) was added lithium hydroxide (10 mg, 0.422 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 625.29, found 625.36.

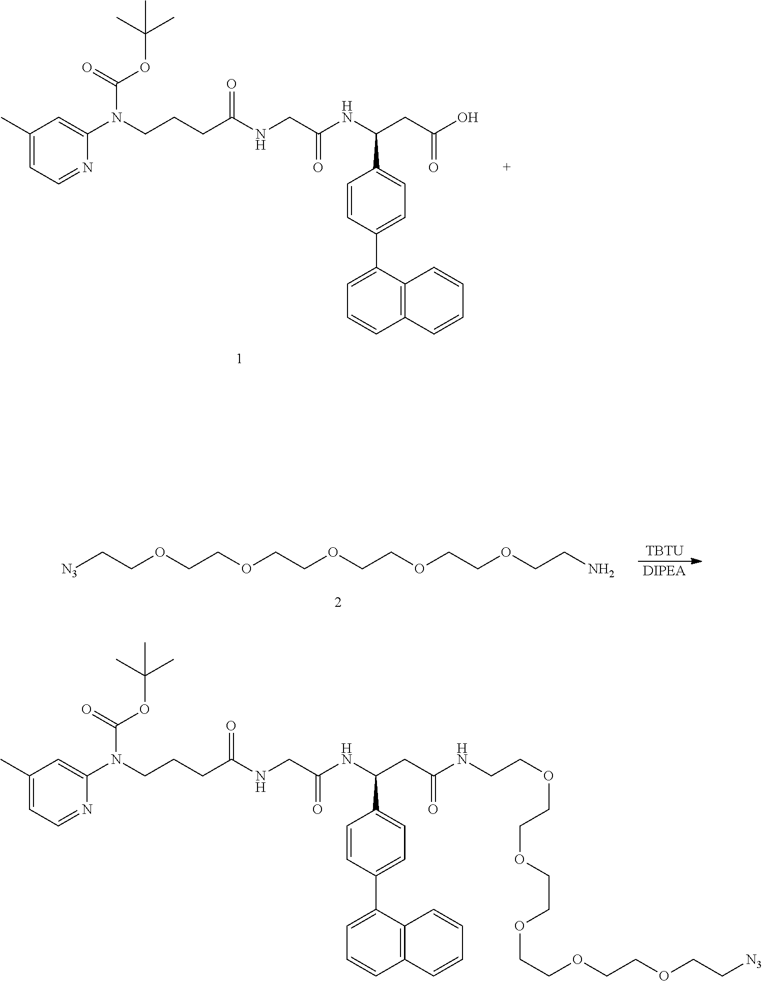 [SMILES: Cc1ccnc(N(CCCC(=O)NCC(=O)N[C@@H](CC(=O)O)c2ccc(-c3cccc4ccccc34)cc2)C(=O)OC(C)(C)C)c1 ; [N-]=[N+]=NCCOCCOCCOCCOCCOCCN ; "TBTU" ; "DIPEA" ; Cc1ccnc(N(CCCC(=O)NCC(=O)N[C@@H](CC(=O)NCCOCCOCCOCCOCCOCCN=[N+]=[N-])c2ccc(-c3cccc4ccccc34)cc2)C(=O)OC(C)(C)C)c1]

To a solution of compound 1 (88 mg, 0.140 mmol, 1 equiv.), compound 2 (48 mg, 0.154 mmol, 1.1 equiv.), and TBTU (54 mg, 0.169 mmol, 1.2 equiv.) in anhydrous DMF (3 mL) was added diisopropylethylamine (0.074 mL, 0.422 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 4-6% methanol in DCM. LC-MS: calculated [M+H]+ 913.47, found 913.70.

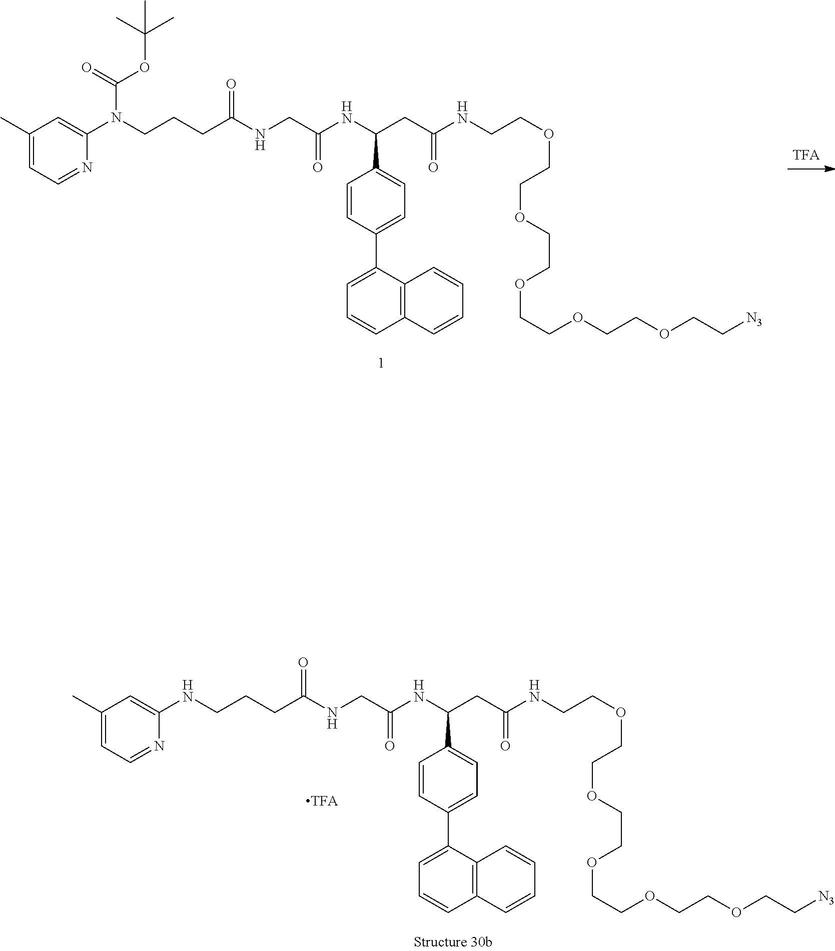 [SMILES: Cc1ccnc(N(CCCC(=O)NCC(=O)N[C@@H](CC(=O)NCCOCCOCCOCCOCCOCCN=[N+]=[N-])c2ccc(-c3cccc4ccccc34)cc2)C(=O)OC(C)(C)C)c1 ; "·TFA" ; Cc1ccnc(NCCCC(=O)NCC(=O)N[C@@H](CC(=O)NCCOCCOCCOCCOCCOCCN=[N+]=[N-])c2ccc(-c3cccc4ccccc34)cc2)c1]

To a solution of compound 1 (93 mg, 0.101 mmol, 1.0 equiv.) in DCM (2 mL) was added TFA (3 mL) and the mixture was stirred at room temperature for another 3 hr. The solvent was removed by rotary evaporator and the product was separated by CombiFlash® using silica gel as the stationary phase. The product was eluted with 10-12% methanol in dichloromethane. LC-MS: calculated [M+H]+ 813.42, found 813.68.

### Synthesis of Structure 31b ((S)-3-(4-(4-((14-azido-3,6,9,12-tetraoxatetradecyl)oxy)naphthalen-1-yl)phenyl)-3-((S)-3-hydroxy-2-(4-((4-methylpyridin-2-yl)amino)butanamido)propanamido)propanoic acid)

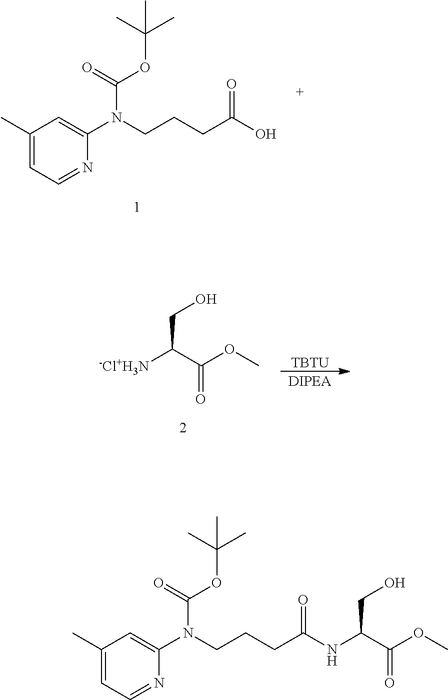

To a solution of compound 1 (150 mg, 0.509 mmol, 1 equiv.), compound 2 (87 mg, 0.560 mmol, 1.1 equiv.), and TBTU (196 mg, 0.196 mmol, 1.2 equiv.) in anhydrous DMF (3 mL) was added diisopropylethylamine (0.074 mL, 0.422 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 4-6% methanol in DCM. LC-MS: calculated [M+H]+ 396.21, found 396.17.

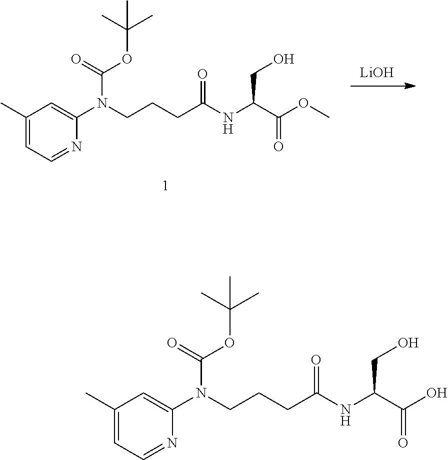

To a solution of compound 1 (196 mg, 0.495 mmol, 1 equiv.) in THE (5 mL) and H₂O (5 mL) was added lithium hydroxide (35 mg, 1.486 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 382.19, found 382.13.

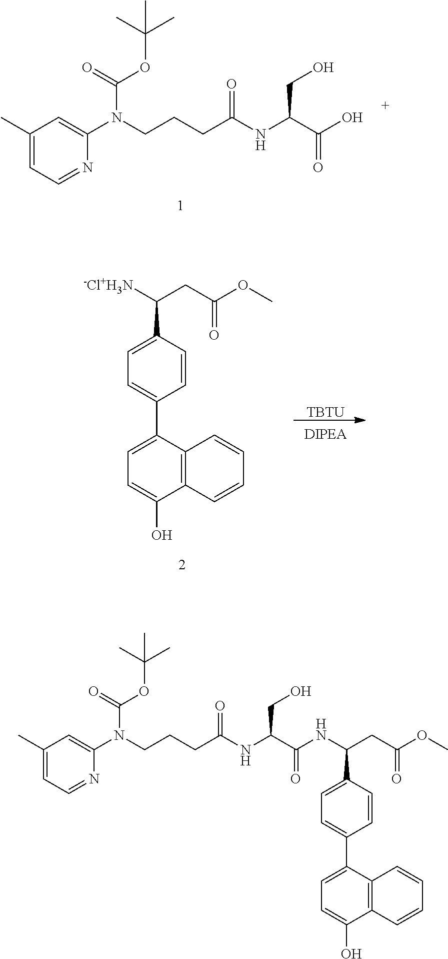

To a solution of compound 1 (189 mg, 0.495 mmol, 1 equiv.), compound 2 (195 mg, 0.545 mmol, 1.1 equiv.), and TBTU (190 mg, 0.595 mmol, 1.2 equiv.) in anhydrous DMF (5 mL) was added diisopropylethylamine (0.259 mL, 1.486 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×10 mL). The organic phase was combined, dried over Na₂SO₄, and concentrated. The product was separated by CombiFlash® using silica gel as the stationary phase and was eluted with 4-6% methanol in DCM. LC-MS: calculated [M+H]+ 685.32, found 685.58.

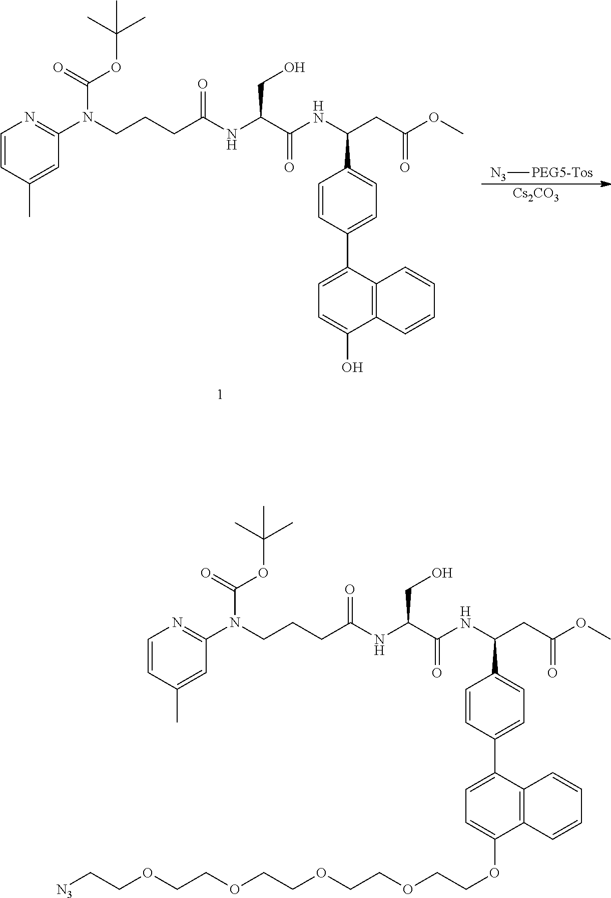

To a solution of compound 1 (75 mg, 0.109 mmol, 1 equiv.) and azido-PEG₅-OTs (91 mg, 0.219 mmol, 2 equiv.) in anhydrous DMF (2 mL) was added Cs₂CO₃ (71 mg, 0.219 mmol, 2 equiv.) at room temperature. The reaction mixture was stirred overnight at 40° C. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4% methanol in DCM. The yield is 29%. LC-MS: calculated [M+H]+ 930.45, found 930.90.

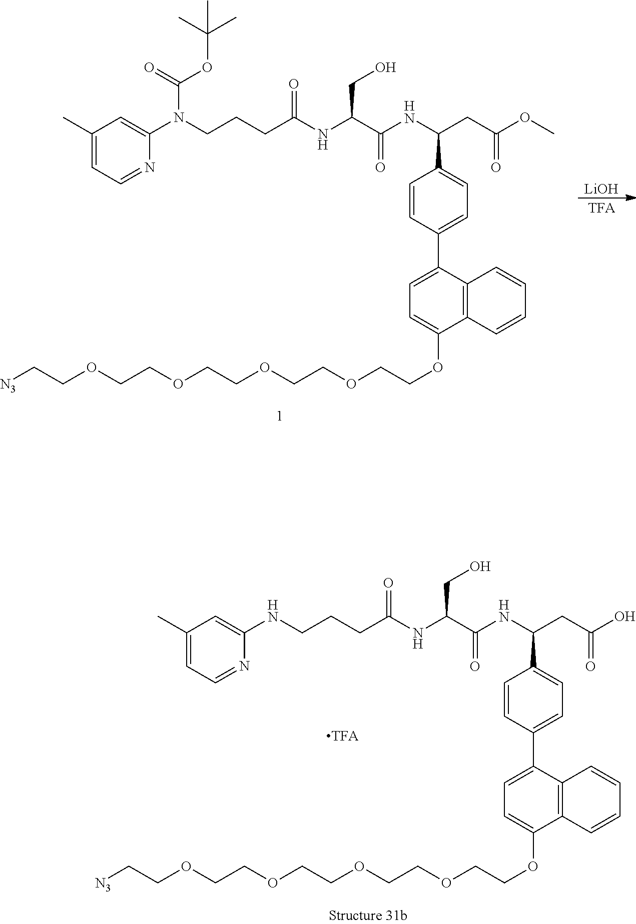 [SMILES: COC(=O)C[C@H](NC(=O)[C@H](CO)NC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)N[C@@H](CO)C(=O)N[C@@H](CC(=O)O)c2ccc(-c3ccc(OCCOCCOCCOCCOCCN=[N+]=[N-])c4ccccc34)cc2)c1 ; "TFA"]

To a solution of compound 1 (30 mg, 0.0323 mmol, 1.0 equiv.) in THE (1 mL) and water (1 mL) was added lithium hydroxide (2.3 mg, 0.0968 mmol, 3.0 equiv.) at room temperature. The mixture was stirred at room temperature for another 1 hrs. The pH was adjusted to 3.0 by HCl (6N) and the aqueous phase was extracted with EtOAc (3×10 mL). The organic phase was combined, dried over Na₂SO₄, and concentrated. TFA (2 mL) and DCM (1 mL) was added into the residue and the mixture was stirred at room temperature for another 3 hr. The solvent was removed by rotary evaporator and the product was separated by CombiFlash® using silica gel as the stationary phase. The product was eluted with 12-15% methanol in dichloromethane. LC-MS: calculated [M+H]+ 816.39, found 816.92.

### Synthesis of Structure 32b ((S)-4-(((S)-1-(4-(4-((14-azido-3,6,9,12-tetraoxatetradecyl)oxy)naphthalen-1-yl)phenyl)-2-carboxyethyl)amino)-3-(4-((4-methylpyridin-2-yl)amino)butanamido)-4-oxobutanoic acid)

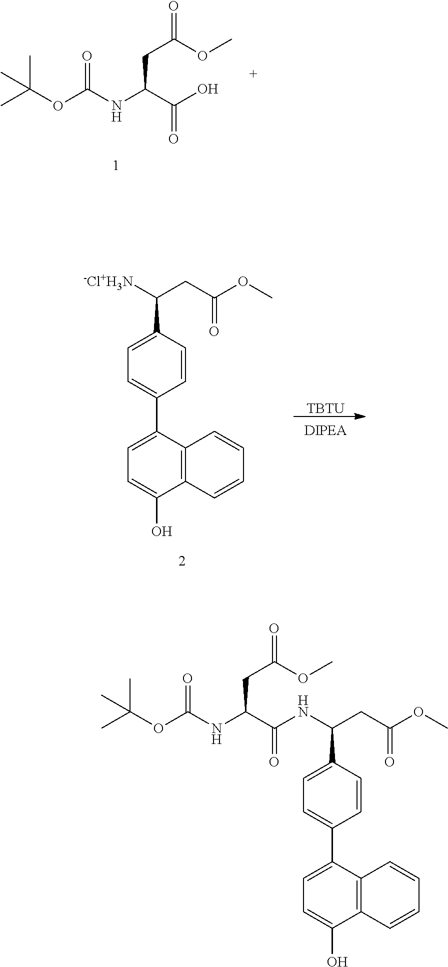

To a solution of compound 1 (100 mg, 0.404 mmol, 1 equiv.), compound 2 (160 mg, 0.444 mmol, 1.1 equiv.), and TBTU (155 mg, 0.485 mmol, 1.2 equiv.) in anhydrous DMF (2 mL) was added diisopropylethylamine (0.211 mL, 1.213 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 2-3% methanol in DCM. LC-MS: calculated [M+H]+ 551.23, found 551.45.

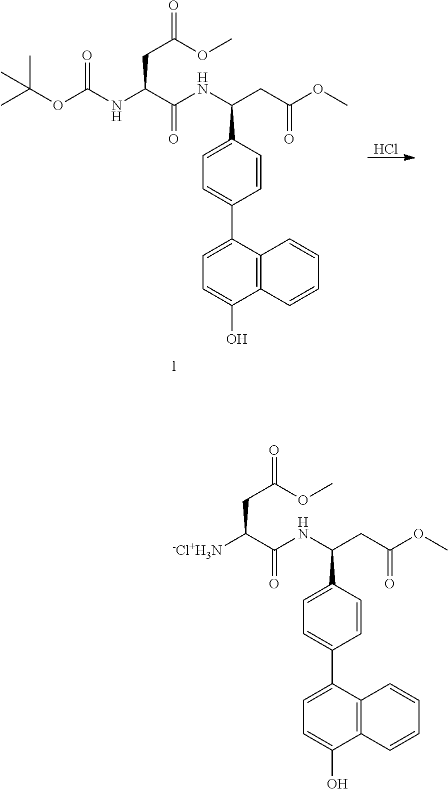

Compound 1 (0.164 g, 0.297 mmol, 1.0 equiv.) was cooled by ice bath. HCl in dioxane (0.745 mL, 2.978 mmol, 10 equiv.) was added into the flask. The reaction was warmed to room temperature and stirred for another 1 hr. The solvent was removed by rotary evaporator and the product was directly used without further purification. LC-MS: calculated [M+H]+ 451.18, found 451.35.

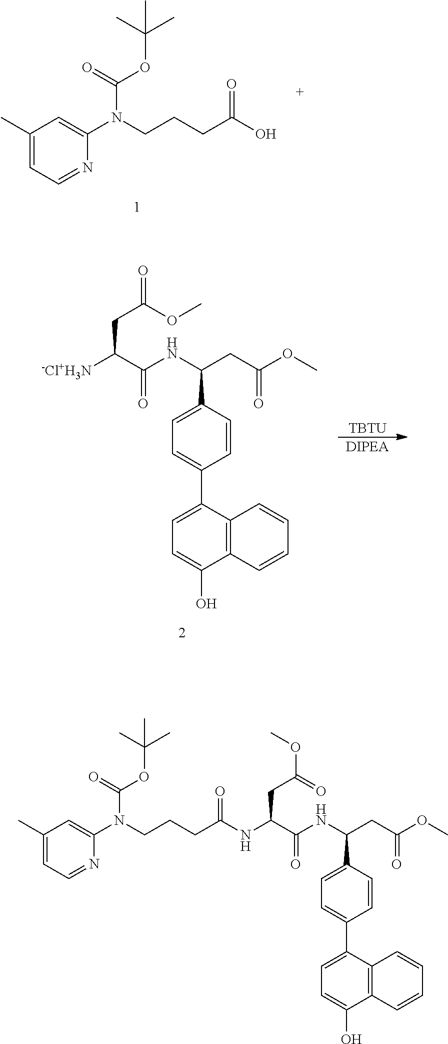

To a solution of compound 1 (100 mg, 0.404 mmol, 1 equiv.), compound 2 (160 mg, 0.444 mmol, 1.1 equiv.), and TBTU (155 mg, 0.485 mmol, 1.2 equiv.) in anhydrous DMF (2 mL) was added diisopropylethylamine (0.211 mL, 1.213 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 3-5% methanol in DCM. LC-MS: calculated [M+H]+ 727.33, found 727.53.

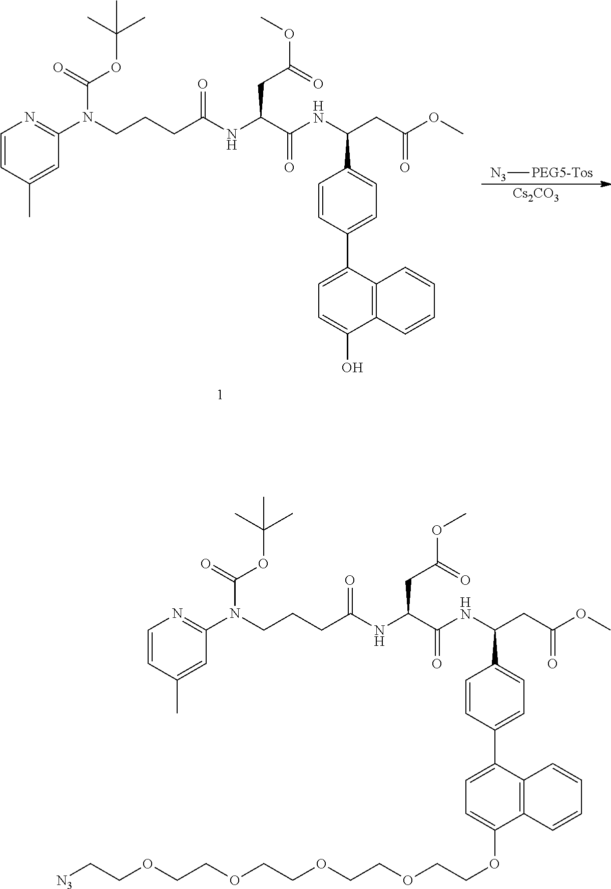

To a solution of compound 1 (150 mg, 0.206 mmol, 1 equiv.) and azido-PEG5-OTs (172 mg, 0.412 mmol, 2 equiv.) in anhydrous DMF (2 mL) was added Cs₂CO₃ (134 mg, 0.412 mmol, 2 equiv.) at room temperature. The reaction mixture was stirred overnight at room temperature. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 4% methanol in DCM. The yield is 29%. LC-MS: calculated [M+H]+ 940.45, found 940.71.

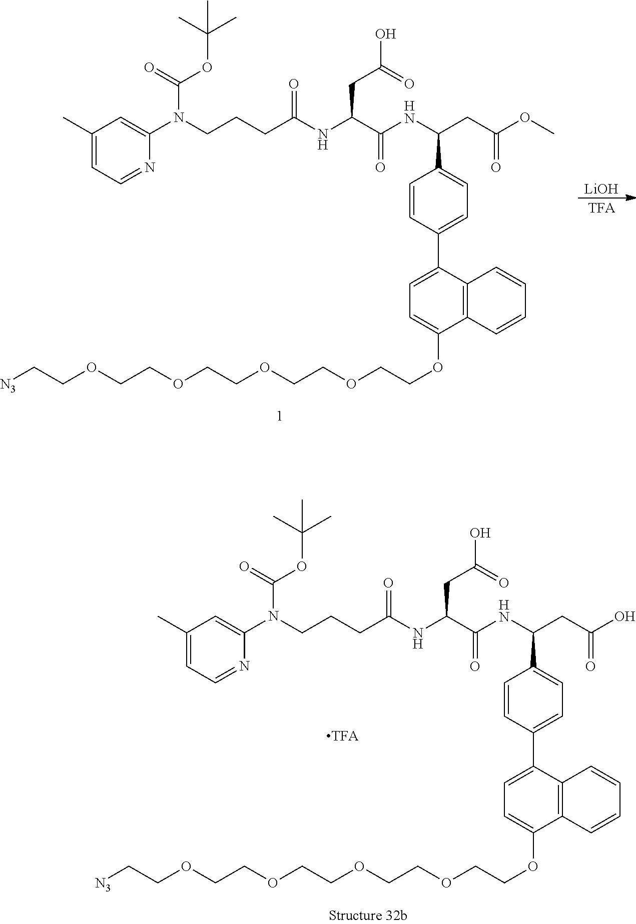 [SMILES: COC(=O)C[C@H](NC(=O)[C@H](CC(=O)O)NC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(N(CCCC(=O)N[C@@H](CC(=O)O)C(=O)N[C@@H](CC(=O)O)c2ccc(-c3ccc(OCCOCCOCCOCCOCCN=[N+]=[N-])c4ccccc34)cc2)C(=O)OC(C)(C)C)c1 ; "TFA"]

To a solution of compound 1 (30 mg, 0.0344 mmol, 1.0 equiv.) in THE (1 mL) and water (1 mL) was added lithium hydroxide (2.5 mg, 0.103 mmol, 3.0 equiv.) at room temperature. The mixture was stirred at room temperature for another 1 hrs. The pH was adjusted to 3.0 by HCl (6N) and the aqueous phase was extracted with EtOAc (3×10 mL). The organic phase was combined, dried over Na₂SO₄, and concentrated. TFA (2 mL) and DCM (1 mL) was added into the residue and the mixture was stirred at room temperature for another 3 hr. The solvent was removed by rotary evaporator and the product was separated by CombiFlash® using silica gel as the stationary phase. The product was eluted with 20% methanol in dichloromethane. LC-MS: calculated [M+H]+ 844.38, found 844.56.

### Synthesis of Structure 33b ((S)-3-((S)-6-amino-2-(4-((4-methylpyridin-2-yl)amino)butanamido)hexanamido)-3-(4-(4-((14-azido-3,6,9,12-tetraoxatetradecyl)oxy)naphthalen-1-yl)phenyl)propanoic acid)

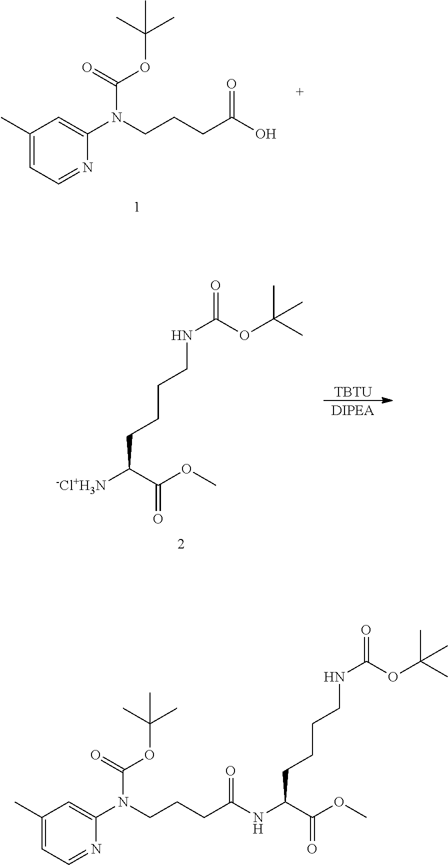

To a solution of compound 1 (150 mg, 0.509 mmol, 1 equiv.), compound 2 (166 mg, 0.560 mmol, 1.1 equiv.), and TBTU (196 mg, 0.611 mmol, 1.2 equiv.) in anhydrous DMF (3 mL) was added diisopropylethylamine (0.266 mL, 1.528 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 3-5% methanol in DCM. LC-MS: calculated [M+H]+ 537.32, found 537.23.

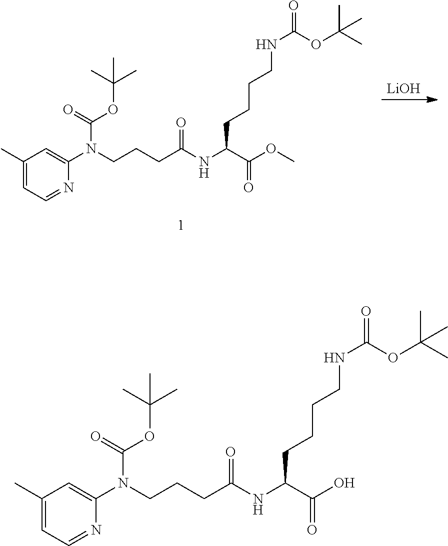

To a solution of compound 1 (230 mg, 0.428 mmol, 1 equiv.) in THE (5 mL) and H₂O (5 mL) was added lithium hydroxide (31 mg, 1.285 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 523.31, found 523.55.

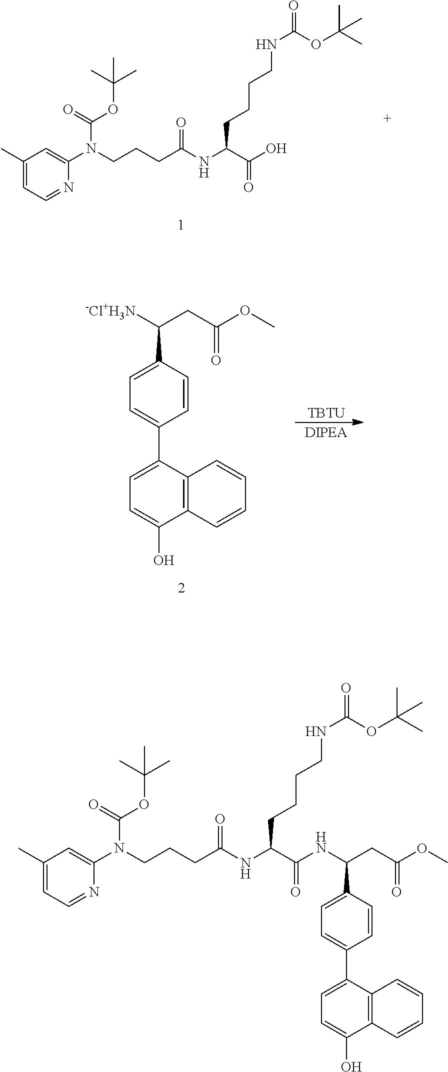

To a solution of compound 1 (230 mg, 0.440 mmol, 1 equiv.), compound 2 (173 mg, 0.484 mmol, 1.1 equiv.), and TBTU (170 mg, 0.528 mmol, 1.2 equiv.) in anhydrous DMF (2 mL) was added diisopropylethylamine (0.230 mL, 1.320 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 4-6% methanol in DCM. LC-MS: calculated [M+H]+ 826.43, found 826.65.

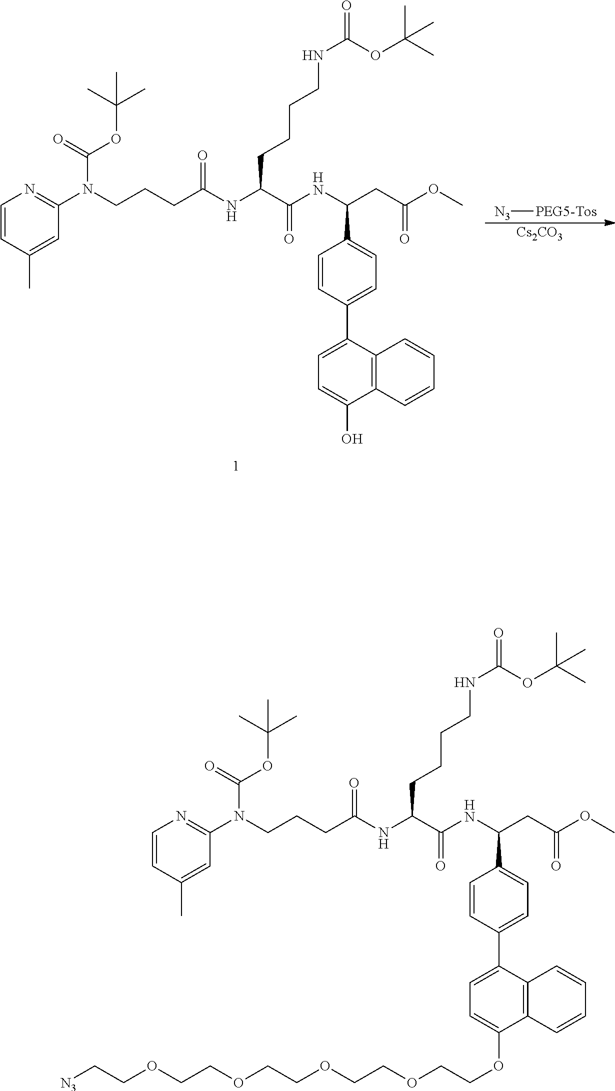

To a solution of compound 1 (150 mg, 0.181 mmol, 1 equiv.) and azido-PEG5-OTs (113 mg, 0.272 mmol, 1.5 equiv.) in anhydrous DMF (2 mL) was added Cs₂CO₃ (118 mg, 0.363 mmol, 2 equiv.) at room temperature. The reaction mixture was stirred at 40° C. for 3 hrs. The reaction was quenched by saturated NaHCO₃ solution (5 mL) and the aqueous layer was extracted with ethyl acetate (3×5 mL). The organic phase was combined, dried over Na₂SO₄, and concentrated. The product was purified by CombiFlash® using silica gel as the stationary phase and was eluted with 4% methanol in DCM. The yield is 66%. LC-MS: calculated [M+H]+ 1071.57, found 1071.89.

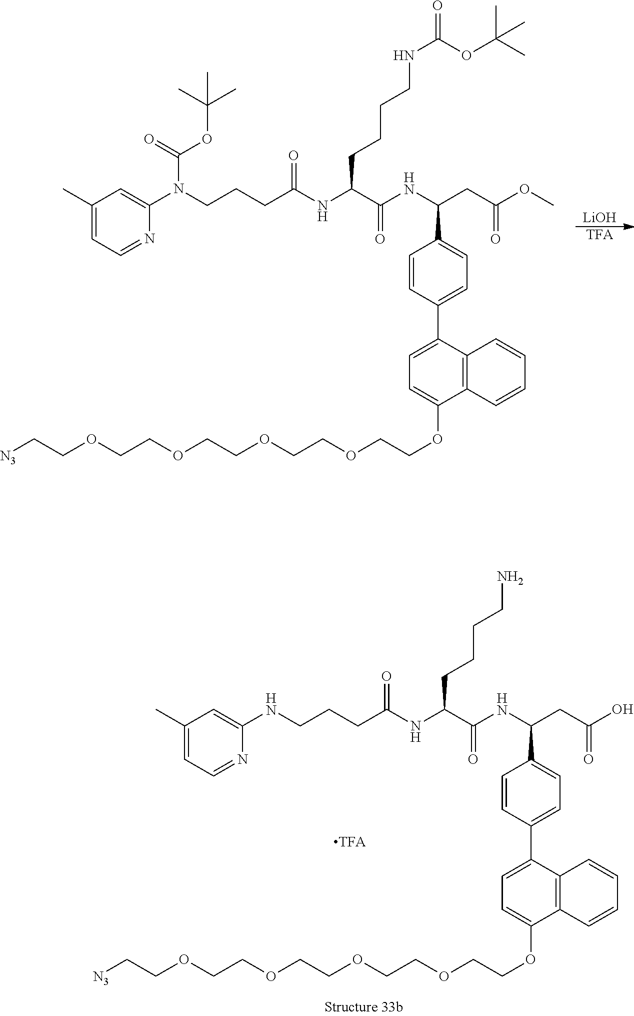

To a solution of compound 1 (130 mg, 0.121 mmol, 1.0 equiv.) in THE (2 mL) and water (2 mL) was added lithium hydroxide (8.7 mg, 0.364 mmol, 3.0 equiv.) at room temperature. The mixture was stirred at room temperature for another 1 hrs. The pH was adjusted to 3.0 by HCl (6N) and the aqueous phase was extracted with EtOAc (3×10 mL). The organic phase was combined, dried over Na₂SO₄, and concentrated. TFA (3 mL) and DCM (2 mL) was added into the residue and the mixture was stirred at room temperature for another 3 hr. The solvent was removed by rotary evaporator and the product was separated by CombiFlash® using silica gel as the stationary phase. The product was eluted with 20% methanol in dichloromethane. LC-MS: calculated [M+H]+ 857.45, found 857.64.

### Synthesis of Structure 34b ((S)-3-(4-(4-((14-azido-3,6,9,12-tetraoxatetradecyl)oxy)naphthalen-1-yl)phenyl)-3-((S)-4-methyl-2-(4-((4-methylpyridin-2-yl)amino)butanamido)pentanamido)propanoic acid)

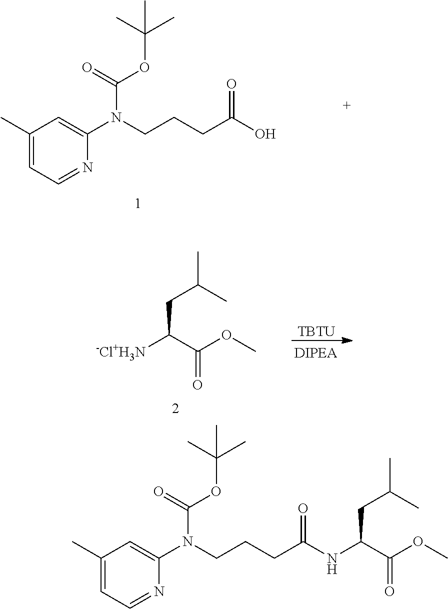

To a solution of compound 1 (150 mg, 0.509 mmol, 1 equiv.), compound 2 (101 mg, 0.560 mmol, 1.1 equiv.), and TBTU (196 mg, 0.611 mmol, 1.2 equiv.) in anhydrous DMF (3 mL) was added diisopropylethylamine (0.266 mL, 1.528 mmol, 3 equiv.) at 0° C. The reaction mixture was warmed to room temperature and stirred for another 1 hr. The reaction was quenched with saturated NaHCO₃ solution (5 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 3-5% methanol in DCM. LC-MS: calculated [M+H]+ 422.26, found 422.36.

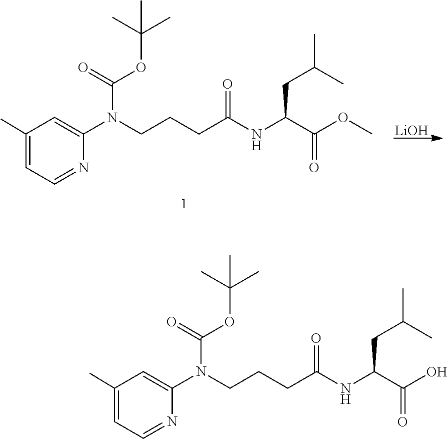

To a solution of compound 1 (186 mg, 0.441 mmol, 1 equiv.) in THE (3 mL) and H₂O (3 mL) was added lithium hydroxide (31 mg, 1.323 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. LC-MS: calculated [M+H]+ 408.24, found 408.23.

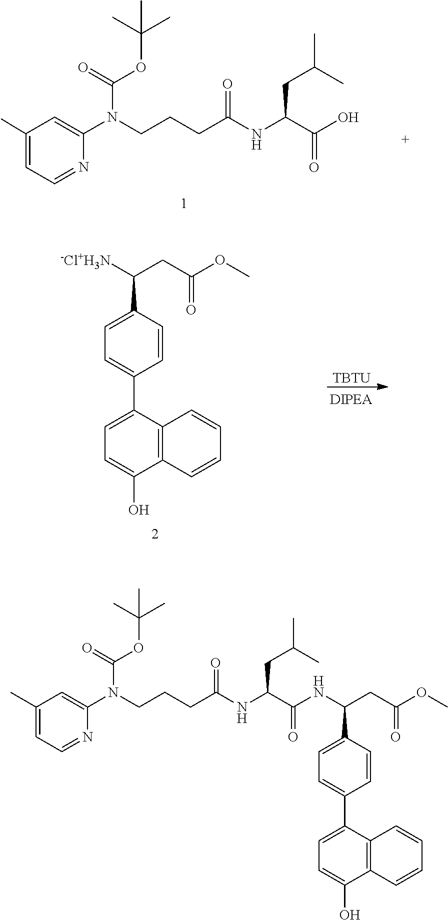

To a solution of compound 1 (168 mg, 0.412 mmol, 1 equiv.), compound 2 (162 mg, 0.453 mmol, 1.1 equiv.), and TBTU (159 mg, 0.494 mmol, 1.2 equiv.) in anhydrous DMF (2 mL) was added diisopropylethylamine (0.215 mL, 1.237 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash® using silica gel as the stationary phase and was eluted with 2-4% methanol in DCM. LC-MS: calculated [M+H]+ 711.37, found 711.69.

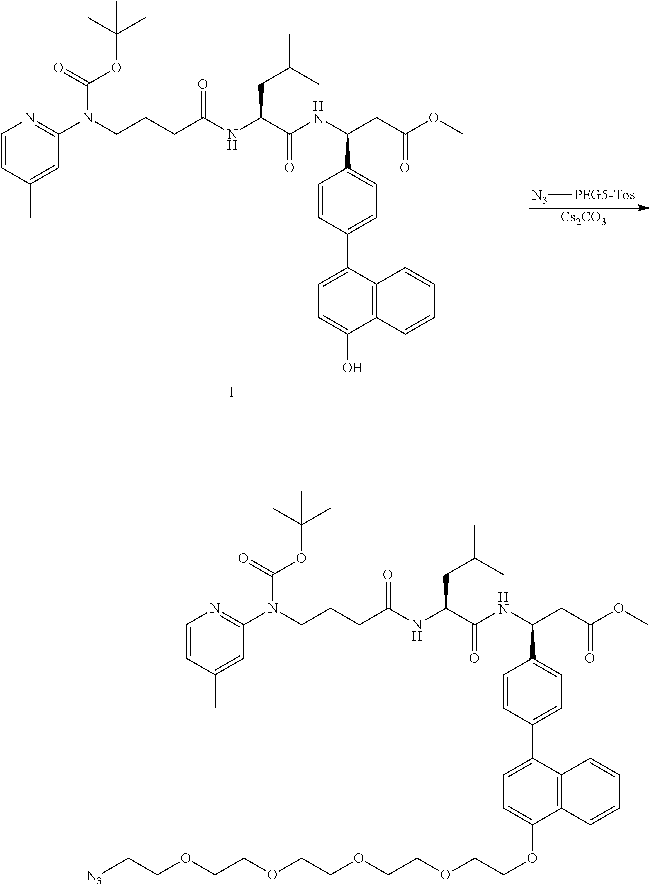

To a solution of compound 1 (150 mg, 0.206 mmol, 1 equiv.) and azido-PEG5-OTs (132 mg, 0.317 mmol, 1.5 equiv.) in anhydrous DMF (2 mL) was added Cs₂CO₃ (137 mg, 0.422 mmol, 2 equiv.) at room temperature. The reaction mixture was stirred at 40° C. for 3 hrs. The reaction was quenched by saturated NaHCO₃ solution (10 mL) and the aqueous layer was extracted with ethyl acetate (3×10 mL). The organic phase was combined, dried over Na₂SO₄, and concentrated. The product was purified by CombiFlash® using silica gel as the stationary phase and was eluted with 3-4% methanol in DCM. The yield is 82%. LC-MS: calculated [M+H]+ 956.51, found 956.64.

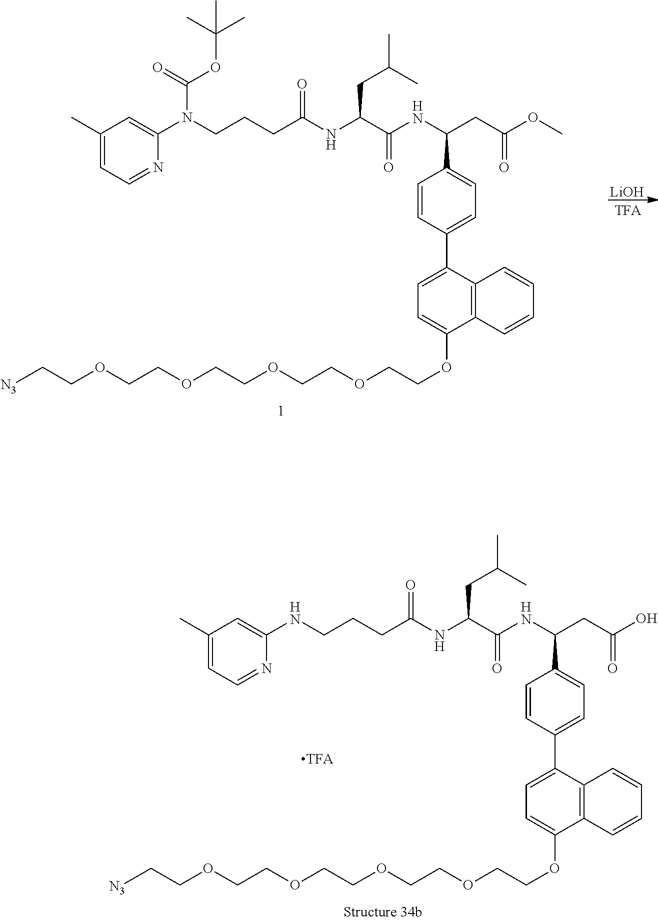 [SMILES: COC(=O)C[C@H](NC(=O)[C@H](CC(C)C)NC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)N[C@@H](CC(C)C)C(=O)N[C@@H](CC(=O)O)c2ccc(-c3ccc(OCCOCCOCCOCCOCCN=[N+]=[N-])c4ccccc34)cc2)c1]

To a solution of compound 1 (160 mg, 0.167 mmol, 1.0 equiv.) in THE (2 mL) and water (2 mL) was added lithium hydroxide (12 mg, 0.502 mmol, 3.0 equiv.) at room temperature. The mixture was stirred at room temperature for another 1 hrs. The pH was adjusted to 3.0 by HCl (6N) and the aqueous phase was extracted with EtOAc (3×10 mL). The organic phase was combined, dried over Na₂SO₄, and concentrated. TFA (3 mL) and DCM (2 mL) was added into the residue and the mixture was stirred at room temperature for another 3 hr. The solvent was removed by rotary evaporator and the product was separated by CombiFlash® using silica gel as the stationary phase. The product was eluted with 8-10% methanol in dichloromethane. LC-MS: calculated [M+H]+ 842.44, found 842.67.

### Synthesis of Structure 35b ((S)-3-(4-(4-((14-azido-3,6,9,12-tetraoxatetradecyl)oxy)naphthalen-1-yl)phenyl)-3-((2S,3R)-3-hydroxy-2-(4-((4-methylpyridin-2-yl)amino)butanamido)butanamido)propanoic acid)

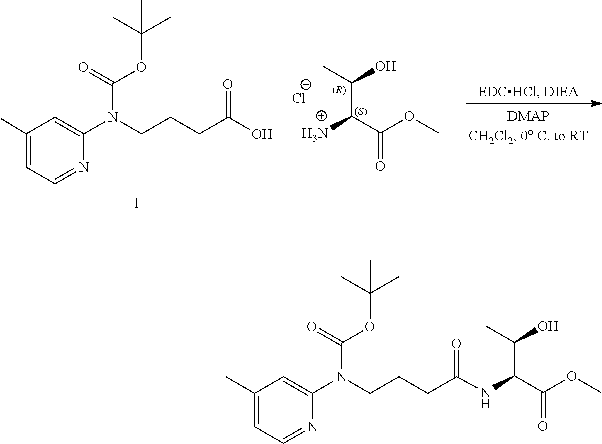

To a vial containing L-threonine-OMe HCl (1.000 g, 5.896 mmol, 1.3 eq) was added compound 1 (1.335 g, 4.535 mmol, 1 eq), dimethylaminopyridine (0.277 g, 2.268 mmol, 0.5 eq), and CH₂Cl₂ (13.3 mL). To the mixture was added diisopropylamine (2.054 mL, 11.792 mmol, 2.6 eq) and the resulting solution was cooled to 0° C. EDC·HCl(1.130 g, 5.896 mmol, 1.3 eq) was added and the reaction was allowed to stir at 0° C. for 30 minutes before warming to room temperature. The reaction was determined to be complete after 16 hours by HPLC and was transferred to a separatory funnel, washed with 66% saturated NH₄C₁(4×20 mL) and saturated NH₄Cl (20 mL). The organic layer was dried over Na₂SO₄ and concentrated to yield a viscous oil (1.7588 g, 94.7%) which was carried directly into the next step. LC-MS: calculated [M+H]⁺: 410.22, found 410.03

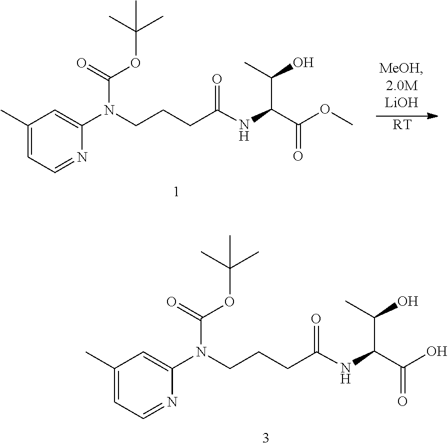

Compound 1 was dissolved in MeOH (4.5 mL) and to the mixture was added a 2.0 M solution of LiOH (9.1 mL). The reaction was stirred for 1.5 h and concentrated to remove McOH. The mixture was then acidified to pH=4 with 20% KHSO₄ and extracted with EtOAc (3×15 mL). The combined organic was washed with brine (20 mL), dried over Na₂SO₄, and concentrated to obtain 3 as a solid (1.5095 g, 88.9% yield). LC-MS: calculated [M-H]⁻: 394.21, found 394.37. ¹H NMR (400 MHz, Chloroform-d) δ 8.26 (d, 1H), 7.27-7.24 (m, 1H), 7.23 (s, 1H), 6.95 (ddd, 1H), 4.60 (dd, 1H), 4.39 (qd, 1H), 3.97-3.77 (m, 2H), 2.36 (s, 3H), , 2.41-2.23 (m, 2H), 1.98-1.84 (m, 2H), 1.45 (s, 9H), 1.19 (d, 3H).

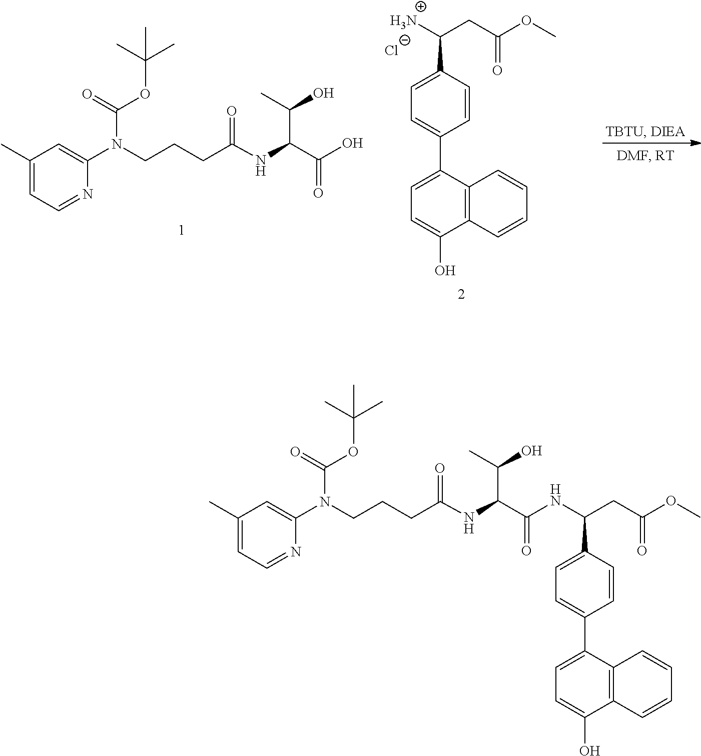

A vial was changed with compound 1 (0.200 g, 0.506 mmol, 1 eq), TBTU (0.195 g, 0.607 mmol, 1.2 eq), DMF (2.0 mL) and DIPEA (0.264 mL, 1.517 mmol, 3.0 eq). The reaction was stirred for 2 minutes before the addition of 2 (0.253 g, 0.708 mmol, 1.4 eq). After completion, the reaction was diluted with sat. aq. NaHCO₃(10 mL), extracted with EtOAc (3×5 mL). The combined organic layers were washed with brine (10 mL), dried over Na₂SO₄, and concentrated. The crude material was purified via column chromatography, eluting with 0-20% MeOH in CH₂Cl₂ to obtain the product (150.8 mg, 42.7% yield). LC-MS: calculated [M+H]⁺: 699.33, found 699.53

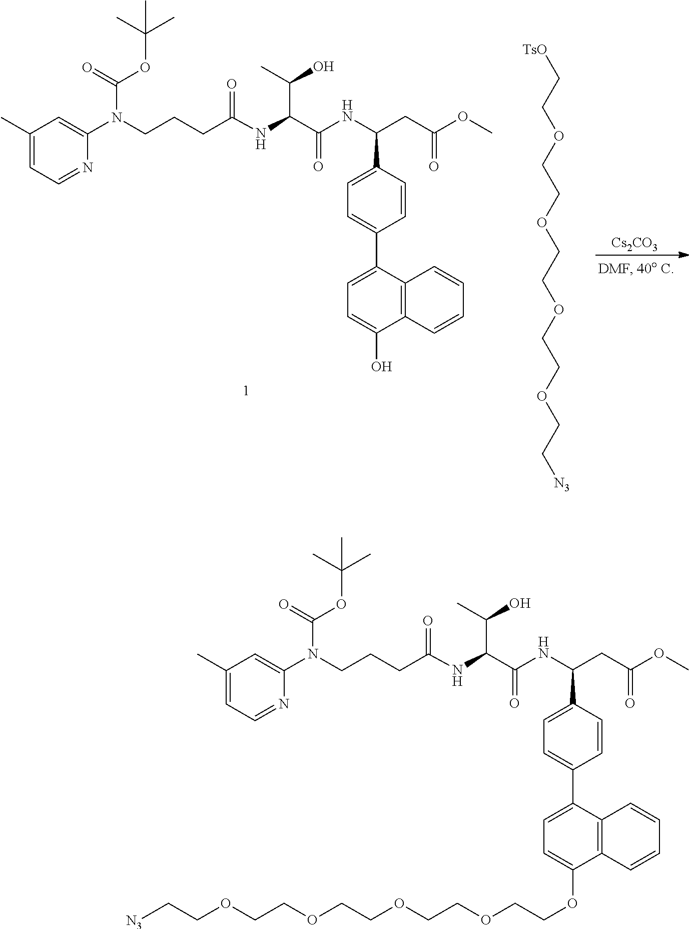

To a vial containing compound 1(0.151 g, 0.216 mmol, 1 eq) was added Cs₂CO₃ (0.106 g, 0.324 mmol, 1.5 eq) and DMF (1.9 mL). N₃-PEG5-OTs (0.135 g, 0.324 mmol, 1.5 eq) was added to the mixture, and the reaction stirred at 40° C. After completion, the reaction was diluted with EtOAc (10 mL), sat. aq. NaHCO₃(5 mL) and water (5 mL). The layers were separated and aqueous extracted a total of 3×10 mL with EtOAc. The combined organic layers were dried over Na₂SO₄ and concentrated. The crude material was purified via column chromatography, eluting with 0-20% MeOH in CH₂Cl₂ to obtain the product (103 mg, 50.4% yield). LC-MS: calculated [M+H]⁺: 944.47, found 944.56

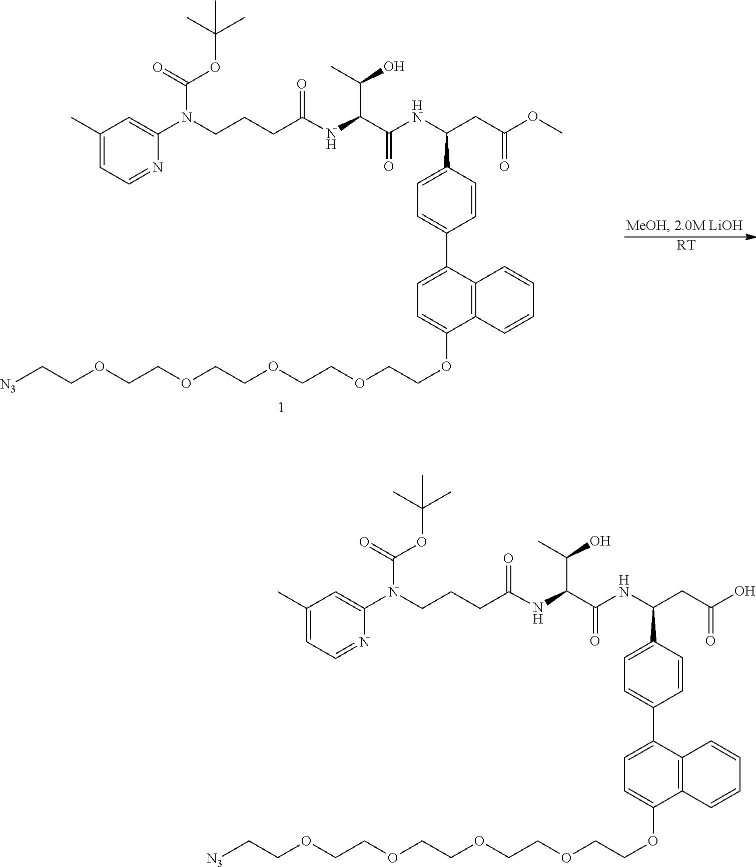 [SMILES: COC(=O)C[C@H](NC(=O)[C@@H](NC(=O)CCCN(C(=O)OC(C)(C)C)c1cc(C)ccn1)[C@@H](C)O)c1ccc(-c2ccc(OCCOCCOCCOCCOCCN=[N+]=[N-])c3ccccc23)cc1 ; "RT" ; Cc1ccnc(N(CCCC(=O)N[C@H](C(=O)N[C@@H](CC(=O)O)c2ccc(-c3ccc(OCCOCCOCCOCCOCCN=[N+]=[N-])c4ccccc34)cc2)[C@@H](C)O)C(=O)OC(C)(C)C)c1]

To a vial containing compound 1 (0.103 g, 0.109 mmol, 1 eq) was added MeOH (1.5 mL) and 2.0 M LiOH (2.0 mL). The reaction was stirred at room temperature, then concentrated to remove MeOH, acidified with 20% KHSO₄ to pH=2. To the mixture was added EtOAc (5 mL) and water (4 mL). The aqueous layer was extracted with EtOAc (3×5 mL). The combined organic layers were washed with brine (10 mL), dried over Na₂SO₄, and concentrated to yield the product (0.0879 g, 86.9%). LC-MS: calculated [M+H]⁺: 930.45, found 930.56.

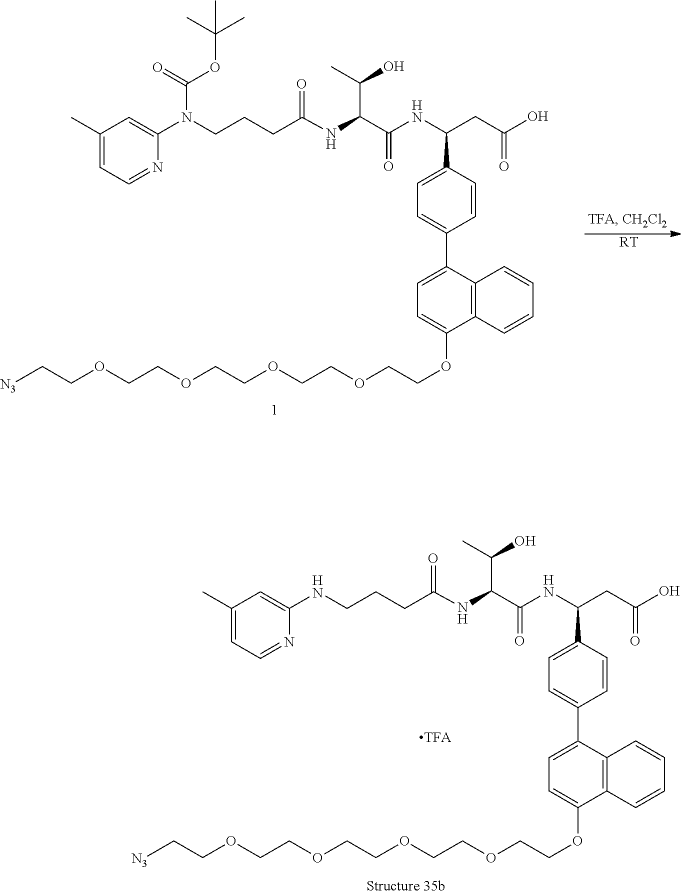 [SMILES: Cc1ccnc(N(CCCC(=O)N[C@H](C(=O)N[C@@H](CC(=O)O)c2ccc(-c3ccc(OCCOCCOCCOCCOCCN=[N+]=[N-])c4ccccc34)cc2)[C@@H](C)O)C(=O)OC(C)(C)C)c1 ; "RT" ; Cc1ccnc(NCCCC(=O)N[C@H](C(=O)N[C@@H](CC(=O)O)c2ccc(-c3ccc(OCCOCCOCCOCCOCCN=[N+]=[N-])c4ccccc34)cc2)[C@@H](C)O)c1]

To a vial containing compound 1(0.0879 g, 0.0945 mmol, 1 eq) was added CH₂Cl₂ (0.3 mL) and trifluoroacetic acid (0.64 mL). The solution was stirred at room temperature. After completion (>97% product), the reaction was concentrated, co-evaporating with toluene (3 mL) and then acetonitrile (2×3 mL). The product was obtained with additional TFA present (115.6 mg).

### Synthesis of Structure 36b ((S)-3-(4-(4-((14-azido-3,6,9,12-tetraoxatetradecyl)oxy)naphthalen-1-yl)phenyl)-3-((2S,3S)-3-methyl-2-(4-((4-methylpyridin-2-yl)amino)butanamido)pentanamido)propanoic acid)

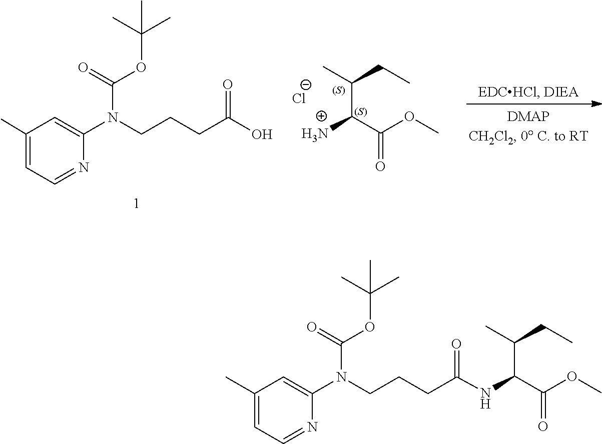

To a vial containing L-isoleucine-OMe HCl (1.000 g, 5.505 mmol, 1.3 eq) was added compound 1(1.246 g, 4.234 mmol, 1 eq), dimethylaminopyridine (0.259 g, 2.117 mmol, 0.5 eq), and CH₂Cl₂ (12.5 mL). To the mixture was added diisopropylamine (2.054 mL, 11.792 mmol, 2.6 eq) and the resulting solution was cooled to 0° C. EDC·HCl(1.055 g, 5.505 mmol, 1.3 eq) was added and the reaction was allowed to stir at 0° C. for 30 minutes before warming to room temperature. The reaction was determined to be complete after 16 hours by HPLC and was transferred to a separatory funnel, washed with 66% saturated NH₄C₁(4×20 mL) and saturated NH₄Cl (1×20 mL). The organic layer was dried over Na₂SO₄ and concentrated to yield a viscous oil (1.8634 g, wet with CH₂Cl₂) which was carried directly into the next step. LC-MS: calculated [M+H]⁺: 422.26, found 422.00.

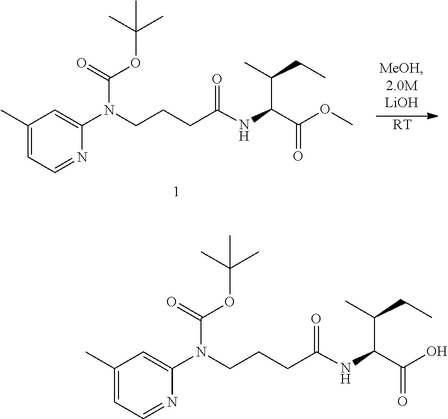

Compound 1 was dissolved in MeOH (4.2 mL) and to the mixture was added a 2.0 mL solution of LiOH (8.5 mL). The reaction was stirred for 1.5 h and concentrated to remove McOH. The mixture was then acidified to pH=4 with 20% KHSO₄ and extracted with EtOAc (3×15 mL). The combined organic was washed with brine (20 mL), dried over Na₂SO₄, and concentrated to obtain the product as a viscous oil (1.6123 g, 93.4% yield across two steps). LC-MS: calculated [M-H]⁻: 406.24, found 406.43. ¹H NMR (400 MHz, Chloroform-d) δ 8.23 (d, 1H), 7.12 (d, 1H), 6.95-6.88 (m, 1H), 4.58 (dd, 1H), 3.99-3.83 (m, 2H), 2.35-2.34 (s, 3H), 2.30 (hept, 2H), 2.00-1.84 (m, 4H), 1.45 (s, 9H), 0.91 (m, 6H).

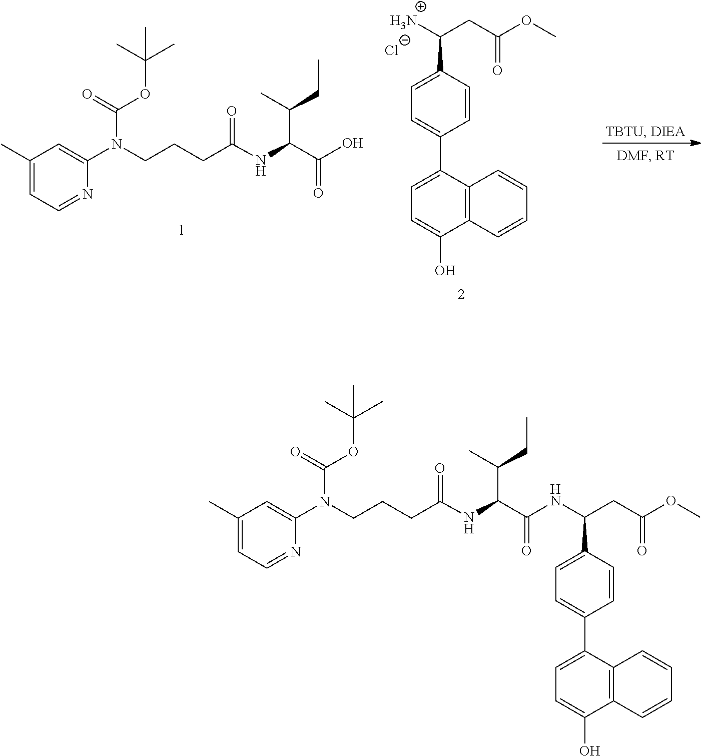

A vial was changed with compound 1 (0.200 g, 0.491 mmol, 1 eq), TBTU (0.189 g, 0.589 mmol, 1.2 eq), DMF (2.0 mL) and DIPEA (0.256 mL, 1.472 mmol, 3.0 eq). The reaction was stirred for 2 minutes before the addition of 2 (0.246 g, 0.687 mmol, 1.4 eq). After completion, the reaction was diluted with sat. aq. NaHCO₃(10 mL), extracted with EtOAc (3×5 mL). The combined organic layers were washed with brine (10 mL), dried over Na₂SO₄, and concentrated. The crude material was purified via column chromatography, eluting with 0-20% MeOH in CH₂Cl₂ to obtain the product (0.3024 mg, 86.7% yield). LC-MS: calculated [M+H]⁺: 711.37, found 711.51.

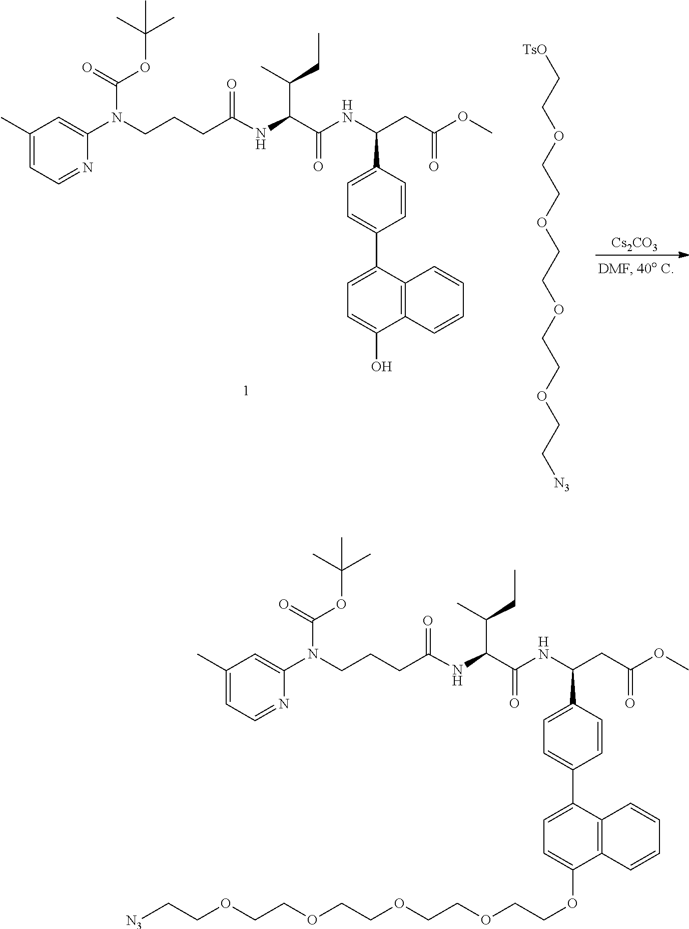

To a vial containing compound 1 (0.170 g, 0.238 mmol, 1 eq) was added Cs₂CO₃ (0.116 g, 0.358 mmol, 1.5 eq) and DMF (2.1 mL). N₃-PEG5-OTs (0.149 g, 0.358 mmol, 1.5 eq) was added to the mixture, and the reaction stirred at 40° C. After completion, the reaction was diluted with EtOAc (10 mL), sat. aq. NaHCO₃(5 mL) and water (5 mL). The layers were separated and aqueous extracted a total of 3×10 mL with EtOAc. Combined organic layers were dried over Na₂SO₄ and concentrated. The crude material was purified via column chromatography, eluting with 0-20% MeOH in CH₂Cl₂ to obtain the product (0.1645 g, 72.1% yield). LC-MS: calculated [M+H]⁺: 956.51, found 956.78.

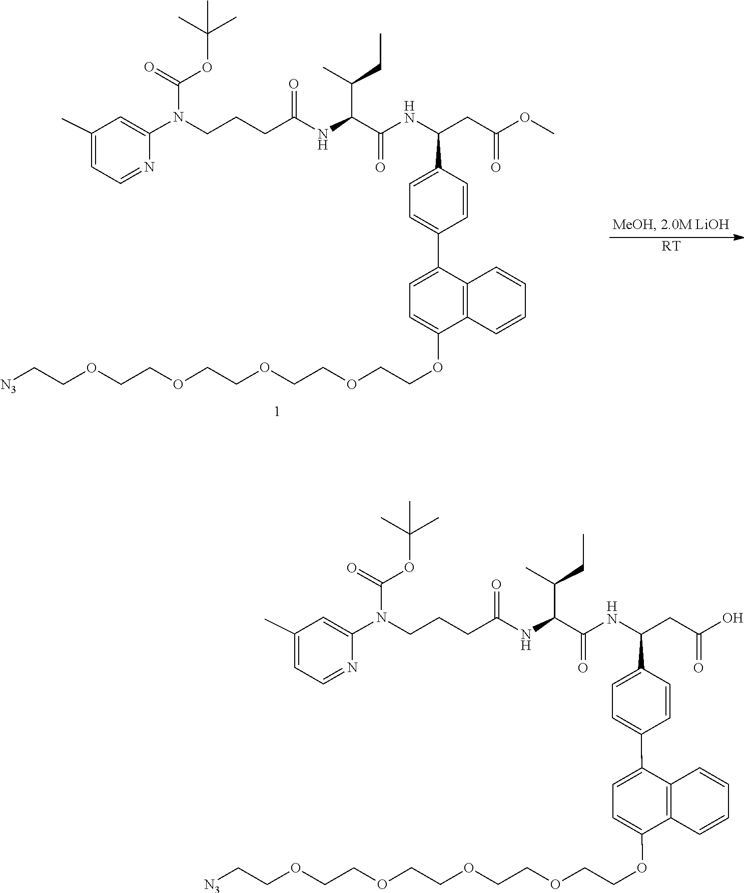 [SMILES: CC[C@H](C)[C@H](NC(=O)CCCN(C(=O)OC(C)(C)C)c1cc(C)ccn1)C(=O)N[C@@H](CC(=O)OC)c1ccc(-c2ccc(OCCOCCOCCOCCOCCN=[N+]=[N-])c3ccccc23)cc1 ; "RT" ; CC[C@H](C)[C@H](NC(=O)CCCN(C(=O)OC(C)(C)C)c1cc(C)ccn1)C(=O)N[C@@H](CC(=O)O)c1ccc(-c2ccc(OCCOCCOCCOCCOCCN=[N+]=[N-])c3ccccc23)cc1]

To a vial containing compound 1 (0.164 g, 0.172 mmol, 1 eq) was added MeOH (2.0 mL) and 2.0 M LiOH (3.0 mL). The reaction was stirred at room temperature and monitored by HPLC. Additional LiOH (33 mg, 1.38 mmol, 8 eq), water (5 mL) and MeOH (4 mL) was required to dissolve the material and drive the reaction. HPLC revealed the formation of two new peaks, thought to be diastereomers. Upon reaching >94% conversion, the reaction was concentrated to remove MeOH, acidified with 20% KHSO₄ to pH=2. To the mixture was added EtOAc (5 mL) and water (4 mL). The aqueous layer was extracted with EtOAc (4×5 mL). The combined organic layers were washed with brine (10 mL), dried over Na₂SO₄, and concentrated to yield the product (0.1417 g, 87.4%). LC-MS: calculated [M+H]⁺: 942.49, found 942.56.

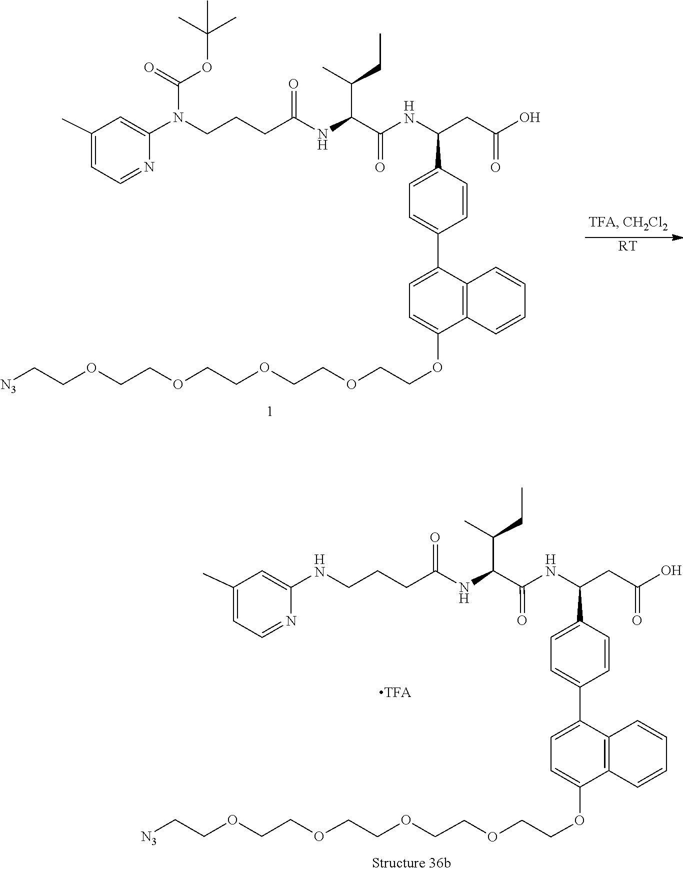 [SMILES: CC[C@H](C)[C@H](NC(=O)CCCN(C(=O)OC(C)(C)C)c1cc(C)ccn1)C(=O)N[C@@H](CC(=O)O)c1ccc(-c2ccc(OCCOCCOCCOCCOCCN=[N+]=[N-])c3ccccc23)cc1 ; "RT" ; CC[C@H](C)[C@H](NC(=O)CCCNc1cc(C)ccn1)C(=O)N[C@@H](CC(=O)O)c1ccc(-c2ccc(OCCOCCOCCOCCOCCN=[N+]=[N-])c3ccccc23)cc1]

To a vial containing compound 1(0.1417 g, 0.1504 mmol, 1 eq) was added CH₂Cl₂ (0.5 mL) and trifluoroacetic acid (1.0 mL). The solution was stirred at room temperature. After completion (>97% product), the reaction was concentrated, co-evaporating with toluene (3 mL) and then acetonitrile (2×3 mL). The product was obtained with additional TFA present (150.3 mg). Two peaks were present through the reaction for both starting material and product. LC-MS: calculated [M+H]⁺: 842.44, found 842.56. Both product peaks were found to have the same mass, indicating the presence of diastereomers.

### Synthesis of Structure 37b ((S)-3-(4-(4-((14-azido-3,6,9,12-tetraoxatetradecyl)oxy)naphthalen-1-yl)phenyl)-3-((R)-3-methyl-2-(4-((4-methylpyridin-2-yl)amino)butanamido)butanamido)propanoic acid)

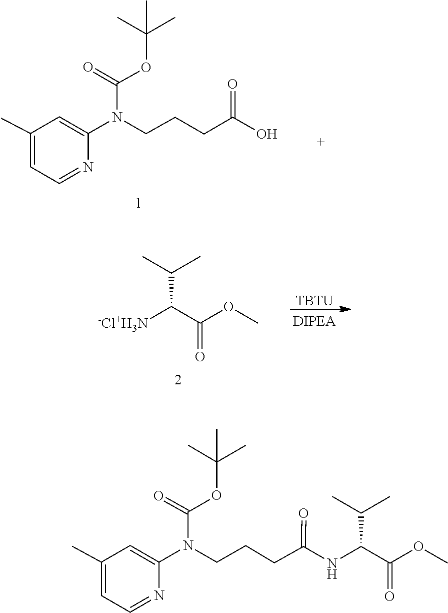

To a solution of compound 1 (150 mg, 0.509 mmol, 1 equiv.), compound 2 (94 mg, 0.560 mmol, 1.1 equiv.), and TBTU (196 mg, 0.611 mmol, 1.2 equiv.) in anhydrous DMF (3 mL) was added diisopropylethylamine (0.266 mL, 1.528 mmol, 3 equiv.) at 0° C. The reaction mixture was warmed to room temperature and stirred for another 1 hr. The reaction was quenched with saturated NaHCO₃ solution (10 mL) and the aqueous phase was extracted with ethyl acetate (3×5 mL). The organic phase was combined, dried over Na₂SO₄, and concentrated. The product was separated by CombiFlash and was eluted with 2-3% methanol in DCM. Yield: 205 mg (99%).

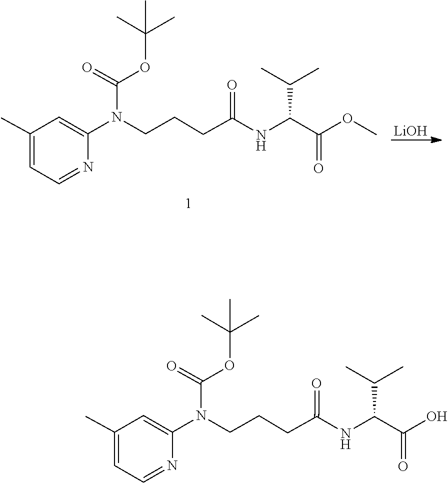

To a solution of compound 1 (207 mg, 0.508 mmol, 1 equiv.) in THE (5 mL) and H₂O (5 mL) was added lithium hydroxide (36 mg, 1.523 mmol, 3 equiv.) portion-wise at 0° C. The reaction mixture was warmed to room temperature. After stirring at room temperature for 1 hr, the reaction mixture was acidified by HCl (6 N) to pH 3.0. The aqueous phase was extracted with ethyl acetate (3×10 mL) and the organic layer was combined, dried over Na₂SO₄, and concentrated. The product was used without further purification. Yield: 180 mg (91%).

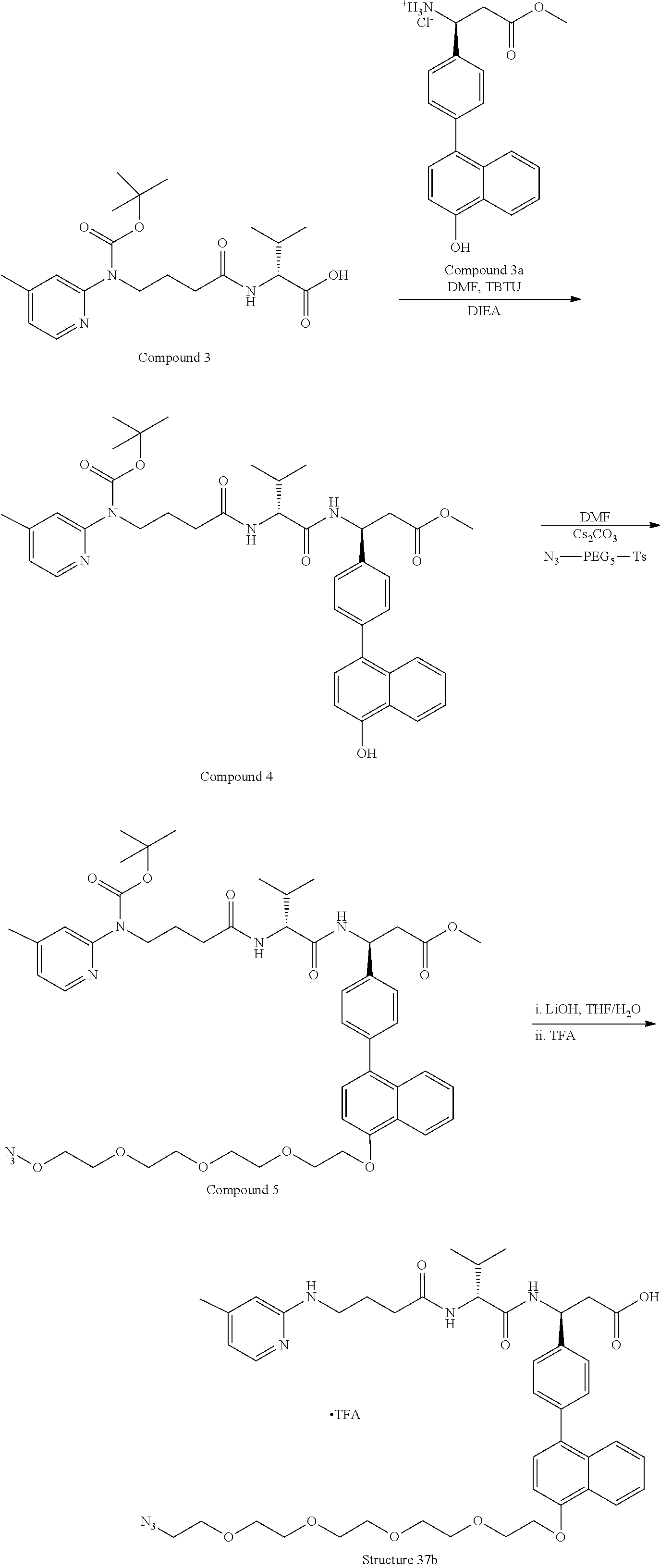

To a solution of compound 3 (180 mg, 0.46 mmol), compound 3a (180 mg, 0.50 mmol), and TBTU (176 mg, 0.55 mmol) in DMF (2.5 mL) was added DIPEA (177 mg, 239 μL, 1.37 mmol) at 0° C. The reaction mixture was warmed to room temperature and stirred for 1 hour. The reaction mixture was quenched with sat. NH₄Cl (aq) solution (1.75 mL) and deionized water (1.75 mL) then extracted with ethyl acetate (8 mL). The aqueous layer was further extracted with ethyl acetate (2×8 mL). The combined organic phase was washed with half sat. NH₄Cl (aq) solution (6 mL) and half sat. NaHCO₃(aq) solution (6 mL). The organic layer was dried over Na₂SO₄, filtered, and concentrated. The crude mixture was separated by CombiFlash using silica gel as the stationary phase with 0-5% methanol in DCM. Yield of compound 4: 295 mg (92%). [M+H]+ calculated for C₄₀H₄₈N₄O7: 697.84, found: 697.82.

To a solution of compound 4 (200 mg, 0.29 mmol) and azido-PEG5-OTs (240 mg, 0.57 mmol) in anhydrous DMF (2.5 mL) was added Cs₂CO₃ (187 mg, 0.57 mmol). The reaction mixture was stirred at 60° C. for 2 hours. The reaction mixture was quenched with sat. NaHCO₃(aq) solution (15 mL) and deionized water (7.5 mL) then extracted with ethyl acetate (10 mL). The aqueous layer was further extracted with ethyl acetate (2×10 mL). The combined organic phase was dried over Na₂SO₄, filtered, and concentrated. The crude mixture was separated by CombiFlash using silica gel as the stationary phase with 0-5% methanol in DCM. Yield of compound 5: 97 mg (36%). [M+H]+ calculated for C₅₀H₆₇N₇O₁₁: 943.15, found: 942.96.

To a solution of compound 5 (94 mg, 0.10 mmol) in THE (1.5 mL) and deionized water (1 mL) was added a solution of lithium hydroxide (7.2 mg, 0.30 mmol) in deionized water (0.5 mL). The reaction mixture was stirred for 1 hour then acidified to pH=3 with 6 M HCl (aq). The aqueous phase was extracted with ethyl acetate (3×5 mL). The combined organic phase was dried over Na₂SO₄, filtered, and concentrated. To the crude residue was added TFA (1.34 mL) and water (67 μL). The reaction mixture was stirred for 1.5 hours at room temperature. The solvent was removed under reduced pressure, and the residue was coevaporated with acetonitrile:toluene [1:1] (2×20 mL). The crude mixture was separated by CombiFlash using silica gel as the stationary phase with 0-10% methanol in DCM. Yield of Structure 37b: 44 mg (53%). [M+H]+ calculated for C₄₄H₅₇N₇O₉: 828.97, found: 828.63.

### Synthesis of Compound 40p, (S)-3-(4-(4-((14-azido-3,6,9,12-tetraoxatetradecyl)oxy)naphthalen-1-yl)phenyl)-3-(2-(3-((4,5-dihydro-1H-imidazol-2-yl)amino)benzamido)acetamido)propanoic acid

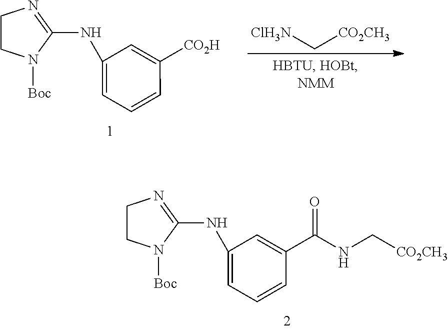

HBTU (239 mg, 0.629 mmol) was added to the ice-cold solution of acid 1 (160 mg, 0.523 mmol), glycine methyl ester hydrochloride (79 mg, 0.639 mmol), HOBt 948 mg, 0.312 mmol), and 4-methylmorpholine (338 uL, 3 mmol) in DMF (10 mL). The cooling bath was removed, and the reaction mixture was stirred for 2h at RT. Water (1 mL) was added and the reaction mixture was concentrated to dryness in high vacuo. The residue was partitioned between EtOAc and water (1:1, 50 mL). EtOAc layer was washed twice with water. The aqueous washes were back-extracted once with EtOAc, organic phases were combined, dried with Na₂SO₄ and concentrated in vacuo, and product was purified on combiflash using the system DCM: 20% MeOH in DCM, gradient 5-30%, 20 min. Yield 192 mg (97%). NMR (DMSO-d₆): 1.5 s (9H); 3.65 s (3H); 3.7 m (4H); 4.0 d (2H), 7.38 t (1H); 7.45 m (1H); 7.96 bs (1H); 8.3 s (1H); 8.88 t (1H); 9.44 bs (1H). Molecular mass calculated: 376.17 Found: MS (ES, pos): 377.30 [M+1]⁺, 277.33 [M+1−Boc]⁺.

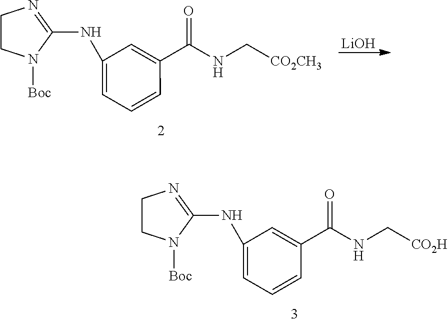 [SMILES: COC(=O)CNC(=O)c1cccc(NC2=NCCN2C(=O)OC(C)(C)C)c1 ; "LiOH" ; CC(C)(C)OC(=O)N1CCN=C1Nc1cccc(C(=O)NCC(=O)O)c1]

A solution of LiOH (36 mg, 1.515 mmol) in water (3 mL) was added dropwise to a stirred solution of the ester 2 in THE (5 mL). Following 2 h of stirring the reaction mixture was cooled in an ice bath and acidified to pH=4.5 with 1N HCl. About ½ of the solvent volume was removed in vacuo and the product was 5 times extracted with EtOAc. Product was dried (Na₂SO₄), filtered, and concentrated and dried in vacuo. Yield 114 mg (63%). The product was used directly in the next step. NMR (DMSO-d₆): 1.51 s (9H); 3.59 m (2H); 3.95 d (2H); 4.05 m (2H); 7.09 m (2H); 7.9 m (2H); 8.92 t (1H); 9.35 bs (1H); 10.52 bs(1H), 12.6 bs (1H).

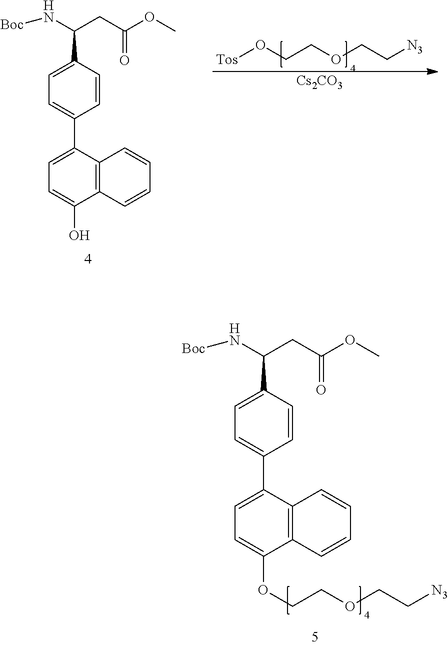

Cesium carbonate (2.556 g, 7.845 mmol) was added into a solution of methyl ester of 3-(N-Boc-amino)-3-[4-[4-hydroxynaphthyl]phenyl]—propionic acid 4 (3 g, 7.132 mmol) and Tos-Peg5-N₃ (3.275 g, 7.845 mmol) in DMF (100 mL). The reaction mixture was stirred at 40° C. for 3h followed by 14h at RT, cooled to 0° C.; and poured into cold saturated solution of NaHCO₃. The product was extracted with 4×200 mL of EtOAc, dried (Na₂SO₄), and concentrated in vacuo. The residual DMF was removed by 2 co-evaporation of toluene from the product on a rotavapor. Combiflash purification using system DCM: 20% MeOH in DCM, gradient=0-20%. Yield 4.757 g (88%). NMR (DMSO-d₆): 1.39 s (9H); 2.80 m(2H); 3.36 t (2H); 3.456 m (12H), 3.69 m (2H); 3.92 m (2H); 4.32 m (2H); 5.03 q (1H); 7.06 d (1H); 7.33 d (1H); 7.40 d (2H); 7.44 d (2H); 7.54 m (3H); 7.77 m (1H); 8.27 m (1H). Molecular mass calculated: 666.33, 684.33 [M+NH₄]⁺ Found MS (ES, pos): 684.54 [M+NH₄]⁺; 567.43 [M+1−Boc]⁺.

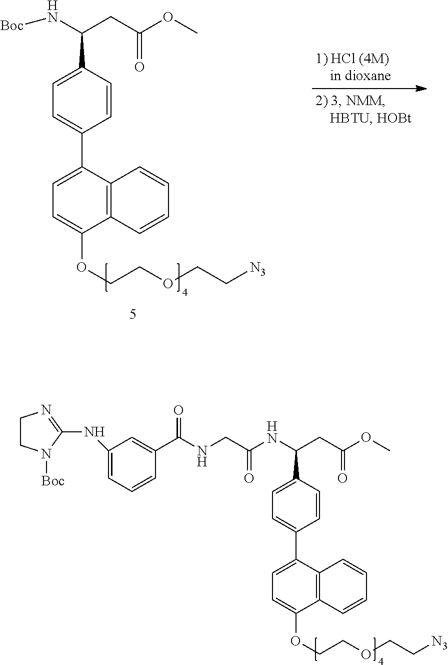

Compound 5 (200 mg, 0.3 mmol) was treated with ice-cold solution of 4M HCl in dioxane, the cooling bath was removed, and the reaction mixture was stirred for 30 min at RT. The product was concentrated and dried in vacuo. The residual HCl was removed by co-evaporation of dioxane. MS (ES, pos): 567 [M+1]⁺. The obtained free amine was dissolved in DMF (10 mL), compound 3 (108 mg, 0.3 mmol), HOBt (28 mg, 0.18 mmol), 4-methylmorpholine (200 uL, 1.8 mmol) were added and the mixture was cooled on an ice bath. HBTU (137 mg, 0.36 mmol) was added, the cooling bath was removed, and the mixture was stirred at RT for 14 h. Water (0.5 mL) was added, DMF was evaporated in high vacuo. The residue was partitioned between EtOAc and water (1:1, 50 mL), basified to pH=8 with NaHCO₃, and the product was extracted with EtOAc 3 times. The EtOAc solution was dried (Na₂SO₄), filtered, and concentrated to dryness. Product was purified on Combiflash® using the system DCM: 20% MeOH in DCM, gradient 0-40%, 20 min. Yield 132 mg (48%). NMR (DMSO-d₆): 1.51 s (9H); 2.60 m (2H); 3.38 t (2H); 3.59 m (2H); 3.95 m 6H); 4.32 m (2H); 5.27 q (1H); 7.06 d (1H); 7.33 d (1H); 7.42 d (2H); 7.48 d (2H); 7.53 m (2H); 7.58 m (2H); 7.77 m (1H); 7.89 m (2H); 8.28 m (1H); 8.62 d (1H); 8.79 t (1H); 9.2 bs (1H). Molecular mass calculated: 910.422 Found MS (ES, pos): 911.58 [M+1]⁺; 811.48 [M+1-Boc]⁺.

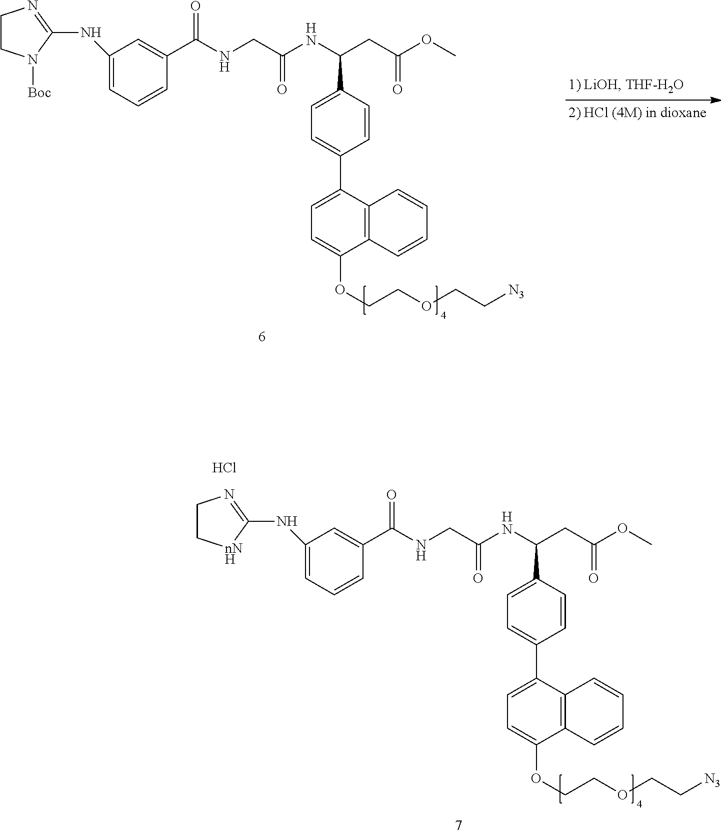

Compound 6 (68.4 mg, 0.075 mmol) was stirred with a solution of LiOH (11 mg, 0.224 mmol) in THF:water=1:1 (2 ml) for 2h at RT. THE was evaporated in vacuo, the aqueous residue was diluted with water to 10 mL, acidified to pH=4 with 1N HCl, brine (3 mL) was added, and the product was extracted 3 times with EtOAc. MS (ES, pos): 897.90 [M+1]⁺; 797.61 [M+1−Boc]⁺. The crude product was treated with ice-cold 4M HCl solution of HCl in dioxane, the cooling bath was removed and the mixture was stirred for 90 min at RT. All volatiles were removed in vacuo, the residual HCl was removed by 2 co-evaporations of dioxane. Yield 59 mg (94%). Molecular mass calculated: 796.35 Found MS (ES, pos): 797.43 [M+1]⁺.

### Synthesis of Compound 41p, (35)-3-(4-(4-((14-azido-3,6,9,12-tetraoxatetradecyl)oxy)naphthalen-1-yl)phenyl)-3-(2-(3-hydroxy-5-((5-hydroxy-1,4,5,6-tetrahydropyrimidin-2-yl)amino)benzamido)acetamido)propanoic acid

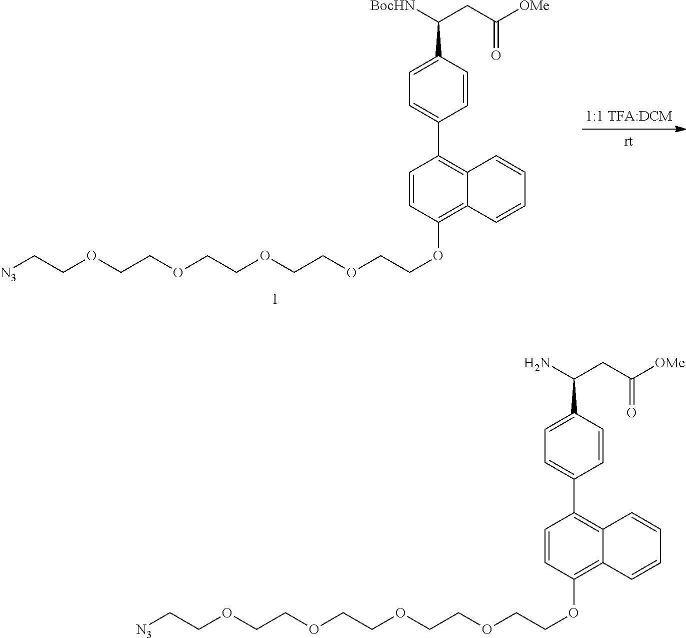 [SMILES: COC(=O)C[C@H](NC(=O)OC(C)(C)C)c1ccc(-c2ccc(OCCOCCOCCOCCOCCN=[N+]=[N-])c3ccccc23)cc1 ; COC(=O)C[C@H](N)c1ccc(-c2ccc(OCCOCCOCCOCCOCCN=[N+]=[N-])c3ccccc23)cc1]

Into a 50-mL round bottom flask with stir bar was added 1.5 g of compound 1, 4 mL of DCM, and 4 mL of TFA. The reaction was allowed to stir under ambient atmosphere at rt at 500 rpm.

After 2 h, the reaction showed full conversion by LC-MS. Reaction was azeotroped with toluene and concentrated under vacuum. The product was subjected to a base extraction with NaHCO₃ and EtOAc to obtain the free amine. LC-MS: calculated [M+H]+ 567.27 m/z, observed 567.52 m/z.

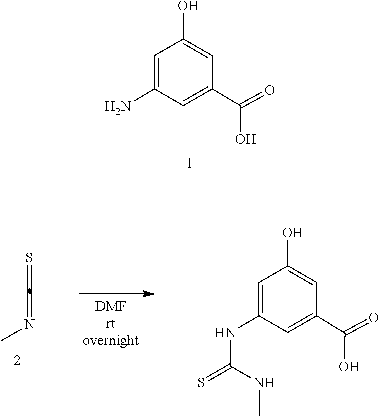

To a solution of compound (4.80 g) 1 in DMF, 2 (2.29 g) was added under a strong flow of N₂(g) via solid-phase transfer; due to 2 sticking to the weight boat, reaction mixture was used to rinse and transfer contents of 2 into reaction flask. The reaction was stirred under ambient conditions for 1-3 h. Upon confirmation of full reaction conversion by LC-MS, crude reaction mixture was carried through to the next step. LC-MS: calculated [M+H]+ 227.04 m/z, observed 227.05 m/z.

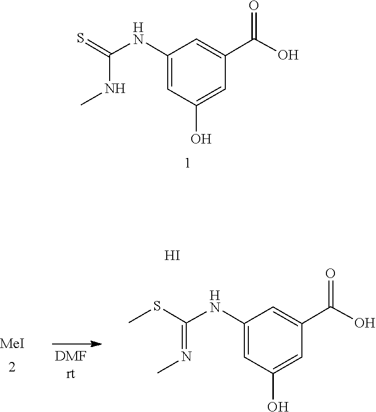

To a solution of compound 1 (0.28 g) in DMF, compound 2 (2.967 mL) was added via syringe and hypodermic needle under ambient conditions (1:15 pm). Reaction was stirred under ambient conditions overnight. Upon confirmation of full reaction conversion by LC-MS, crude reaction mixture was carried through to the next step. LC-MS: calculated [M+H]+ 241.06 m/z, observed 241.00 m/z.

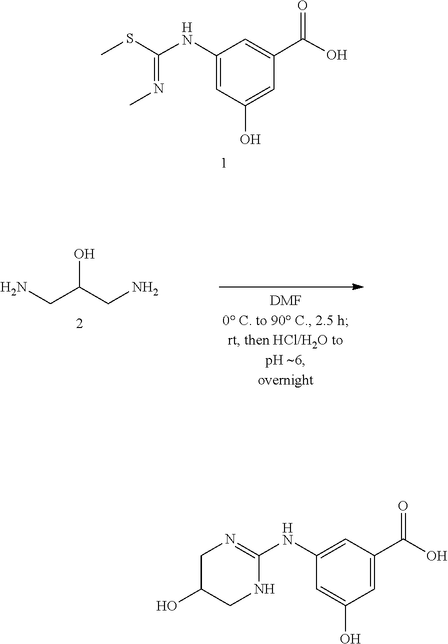 [SMILES: C/N=C(/Nc1cc(O)cc(C(=O)O)c1)SC ; NCC(O)CN ; O=C(O)c1cc(O)cc(NC2=NCC(O)CN2)c1]

To a solution of compound 1 (0.28 g) in DMF, cooled to 0° C., compound 2 (4.60 g) was added under ambient conditions. The reaction was heated to 90° C. and allowed to stir for 3 h. Then reaction mixture was cooled to rt, and water (10 mL) and concentrated HCl were added to adjust reaction pH to 5-6. The reaction was stirred at rt overnight. Upon confirmation of full reaction conversion by LC-MS, reaction mixture was filtered and rinsed with EtOAc to recover a taupe solid product in the filter cake. No product was observed in the filtrate. LC-MS: calculated [M+H]+ 252.09 m/z, observed 252.08 m/z. The isolated product weighed 0.4287 g. Yield over 5 steps: 5.0%.

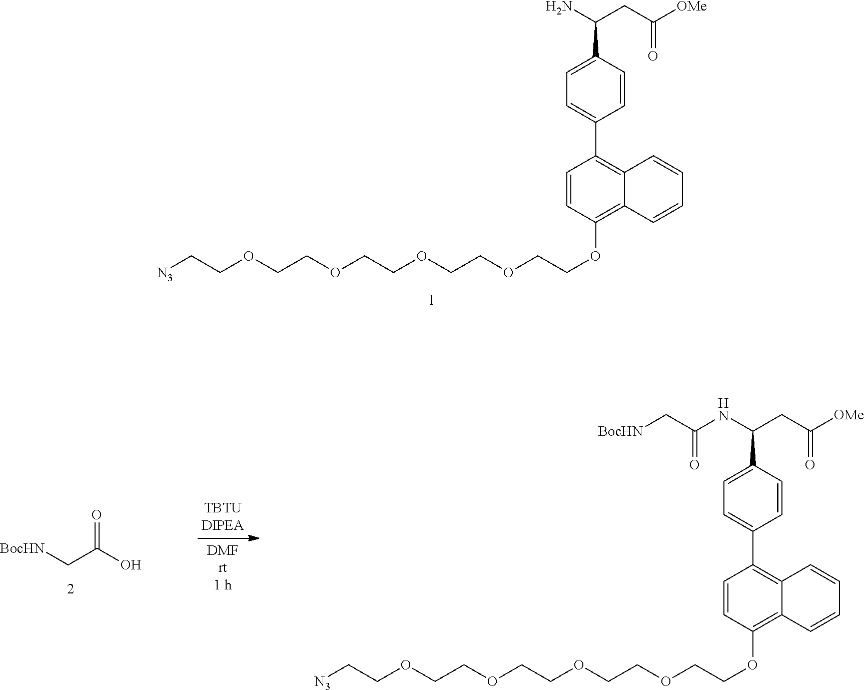 [SMILES: COC(=O)C[C@H](N)c1ccc(-c2ccc(OCCOCCOCCOCCOCCN=[N+]=[N-])c3ccccc23)cc1 ; CC(C)(C)OC(=O)NCC(=O)O ; COC(=O)C[C@H](NC(=O)CNC(=O)OC(C)(C)C)c1ccc(-c2ccc(OCCOCCOCCOCCOCCN=[N+]=[N-])c3ccccc23)cc1]

To a solution of compounds 1 (0.54 g) and 2 (0.25 g) in DMF was added TBTU (0.37 g) and then DIPEA (0.50 mL) under ambient conditions. Reaction was stirred for 3 h. Then reaction mixture was quenched with NaHCO₃(10 mL) and brine (15 mL). The product was extracted with EtOAc (3×15 mL). The combined organic phase was dried over Na₂SO₄, filtered, and concentrated. The residue was purified by CombiFlash® using silica gel as the stationary phase with a gradient of DCM to 20% MeOH in DCM (0-40%), in which product eluted at 13% B. Recovery of product: 0.50 g (71.9% yield). LC-MS: calculated [M+H]+ 724.35 m/z, observed 724.69 m/z.

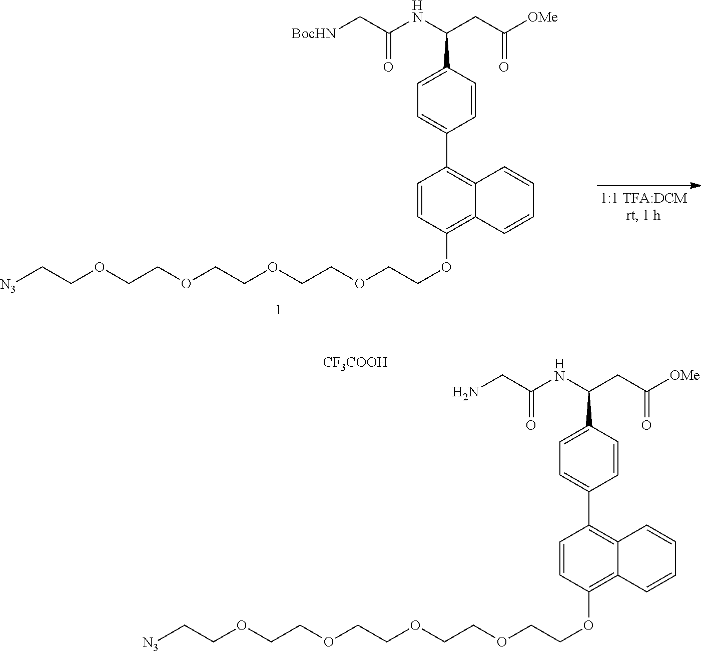 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.50 g) in DCM was added TFA (1.59 mL) at rt. The reaction was stirred under ambient conditions. After 1 h, full conversion was confirmed via LC-MS. The reaction mixture was quenched with NaHCO₃(10 mL), extracted with EtOAc (3×10 mL), and concentrated under vacuum. No isolation was necessary. Concentration provided a yellow oil (0.28 g, 54.8%.) LC-MS: calculated [M+H]+ 624.30 m/z, observed 624.50 m/z.

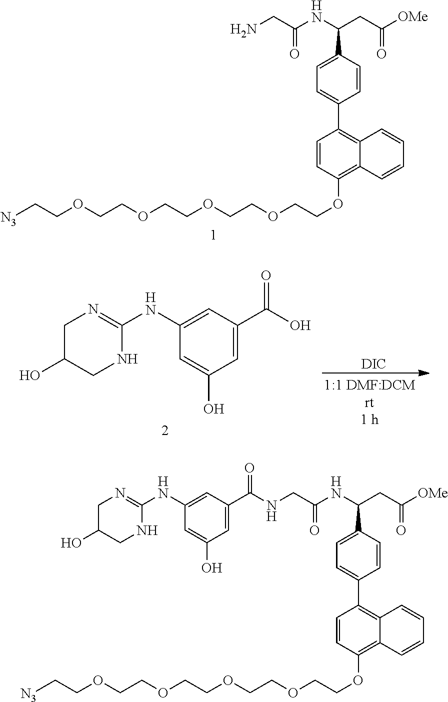 [SMILES: COC(=O)C[C@H](NC(=O)CN)c1ccc(-c2ccc(OCCOCCOCCOCCOCCN=[N+]=[N-])c3ccccc23)cc1 ; O=C(O)c1cc(O)cc(NC2=NCC(O)CN2)c1 ; "DIC" ; COC(=O)C[C@H](NC(=O)CNC(=O)c1cc(O)cc(NC2=NCC(O)CN2)c1)c1ccc(-c2ccc(OCCOCCOCCOCCOCCN=[N+]=[N-])c3ccccc23)cc1]

To a solution of compounds 1 (0.050 g) and 2 (0.0211 g) in 1:1 DMF:DCM under N₂(g) was added DIC (0.015 mL) at rt. Reaction was stirred under N₂(g) at rt over the weekend. By LC-MS, the observed mixture consisted of unreacted starting materials and some urea intermediate. Two equivalents of DIPEA (0.028 mL) were then added. After 40 min., the observed mixture also included undesired side product. After 5 h, no desired product was observed, so reaction was heated to 40° C., and reaction was stirred overnight. With no observation of desired product, DIC (0.1 mL) and HOBt (˜10-20 mg) were added, and reaction was allowed to continue stirring at 40° C. for 1.5 h until full conversion to product was observed. The crude reaction mixture was then employed for the next step in-situ. LC-MS: calculated [M+H]+ 857.38 m/z, observed 857.84 m/z.

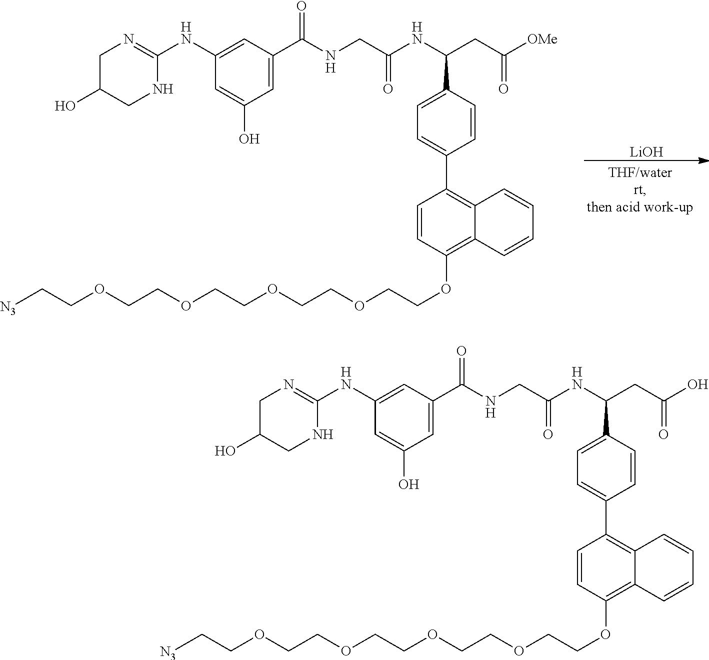 [SMILES: COC(=O)C[C@H](NC(=O)CNC(=O)c1cc(O)cc(NC2=NCC(O)CN2)c1)c1ccc(-c2ccc(OCCOCCOCCOCCOCCN=[N+]=[N-])c3ccccc23)cc1 ; "LiOH" ; [N-]=[N+]=NCCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12]

Saponification was performed in-situ of ester (0.069 g). To the crude reaction mixture were added ˜2 mL of water and then ˜10 mg of LiOH at rt under normal atmosphere. The reaction was stirred at rt until full conversion was observed by LC-MS. Mixture was then concentrated under vacuum and azeotroped with PhMe. The mixture was resuspended in 1 mL of DMF and 1 mL of water and isolated via reverse-phase HPLC. Recovery of compound 41p: 0.029 g (43.0% over two steps). LC-MS: calculated [M+H]+ 843.36 m/z, observed 843.35 m/z.

### Synthesis of Compound 42p, (S)-3-(4-(4-((14-azido-3,6,9,12-tetraoxatetradecyl)oxy)naphthalen-1-yl)phenyl)-3-(2-(5-guanidinopentanamido)acetamido)propanoic acid

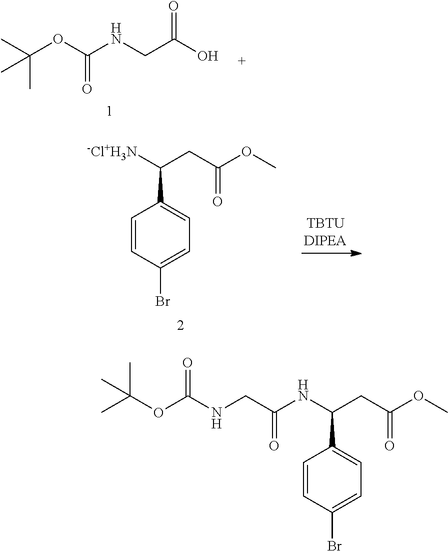

To a solution of compound 1 (1300 mg, 7.42 mmol, 1.0 equiv.), compound 2 (2295 mg, 7.792 mmol, 1.05 equiv.) and diisopropylethylamine (3.878 mL, 22.262 mmol, 3.0 equiv.) in anhydrous DMF (10 mL) was added TBTU (2859 mg, 8.905 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 415.08, found 415.29. Yield: 0.19 g, 6.04%.

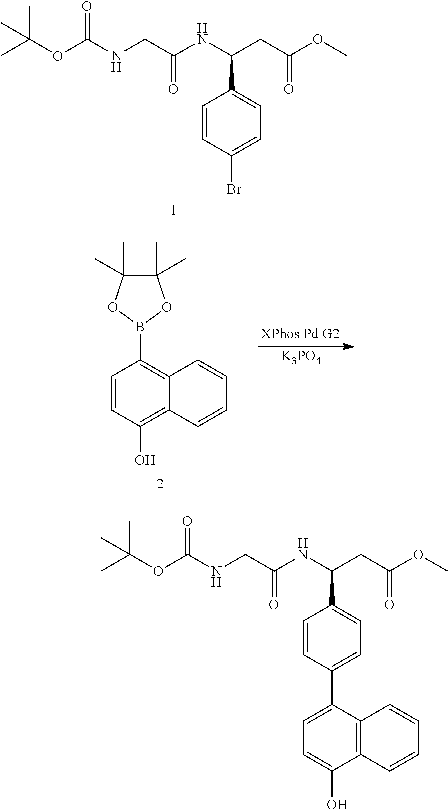

Compound 1 (3.20 g, 7.705 mmol, 1.0 equiv.), compound 2 (3.12 g, 11.558 mmol, 1.5 equiv.), XPhos Pd G2 (121 mg, 0.154 mmol, 0.02 equiv.), and K₃PO₄ (3.27 g, 15.411 mmol, 2.0 equiv.) were mixed in a round-bottom flask. The flask was sealed with a screw-cap septum, and then evacuated and backfilled with nitrogen (this process was repeated a total of 3 times). Then, THE (20 mL) and water (4 mL) were added via syringe. The mixture was bubbled with nitrogen for 10 min and the reaction was kept at 40° C. for 3 hrs. The reaction was quenched with saturated NaHCO₃ aqueous solution (20 mL), and the aqueous phase was extracted with ethyl acetate (3×20 mL). The organic phase was combined, dried over Na₂SO₄, and concentrated. The compound was separated by CombiFlash®, and was eluted with 2-4% methanol in DCM.

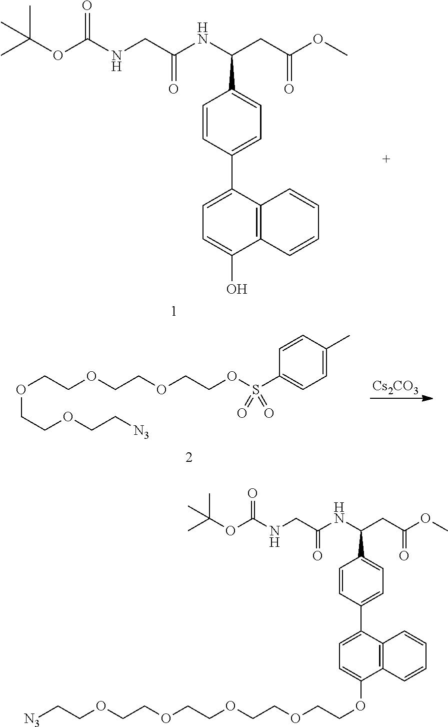

To a solution of compound 1 (1.61 g, 3.364 mmol, 1.0 equiv.) and compound 2 (1.75 g, 4.205 mmol, 1.25 equiv.) in anhydrous DMF (10 mL) was added cesium carbonate (2.19 g, 6.728 mmol, 2.0 equiv.) at room temperature. The reaction was kept at 50° C. for 2 hrs. The reaction was quenched with water (20 mL) and was extracted with ethyl acetate (3×10 mL). The organic phase was combined, dried over anhydrous Na₂SO₄, and concentrated. The product was purified by CombiFlash, and was eluted with 2-4% methanol in dichloromethane. LC-MS: calculated [M+H]+ 724.35, found 724.60.

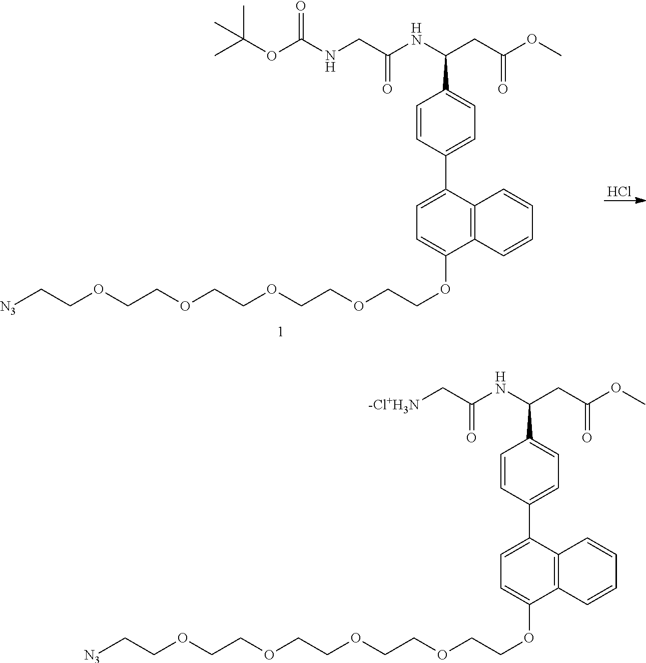

To a solution of compound 1 (1880 mg, 2.597 mmol, 1.0 equiv.) in anhydrous dioxane (3 mL) was added HCl in dioxane (3.25 mL, 12.986 mmol, 5.0 equiv.) at room temperature. The reaction was kept at room temperature for 2 hrs. The solvent was removed and the product was used directly without purification. LC-MS: calculated [M+H]+ 624.30, found 624.41.

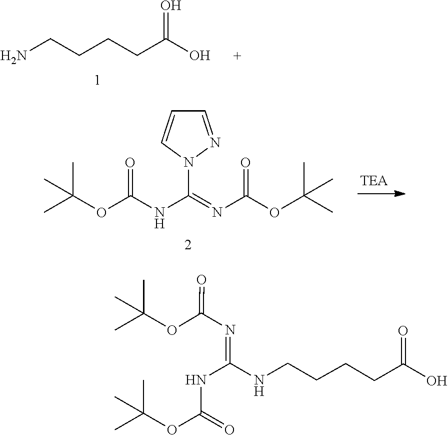

To a solution of compound 1 (500 mg, 4.268 mmol, 1.0 equiv.) and compound 2 (1.607 g, 5.335 mmol, 1.25 equiv.) in anhydrous methanol (10 mL) was added triethylamine (1.786 mL, 12.804 mmol, 3.0 equiv.) at room temperature. The reaction was kept at room temperature overnight. The reaction mixture was concentrated and the product was separated by CombiFlash. The product was eluted with 2-3% methanol in dichloromethane. LC-MS: calculated [M+H]+ 360.21, found 360.46.

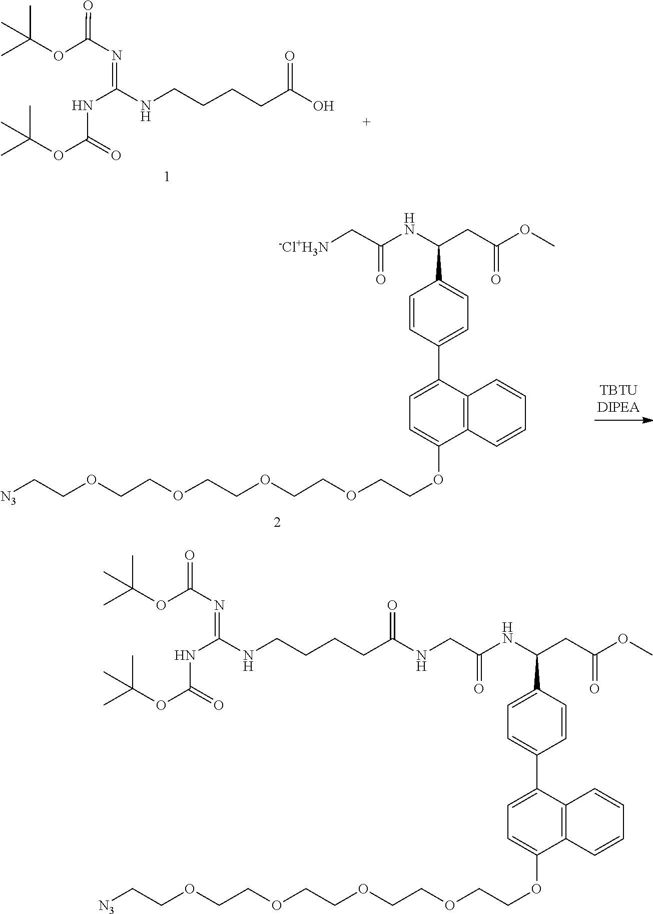

To a solution of compound 1 (66 mg, 0.183 mmol, 1.0 equiv.), compound 2 (127 mg, 0.192 mmol, 1.05 equiv.) and diisopropylethylamine (0.096 mL, 0.550 mmol, 3.0 equiv.) in anhydrous DMF (1 mL) was added TBTU (70 mg, 0.220 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 965.49, found 965.69.

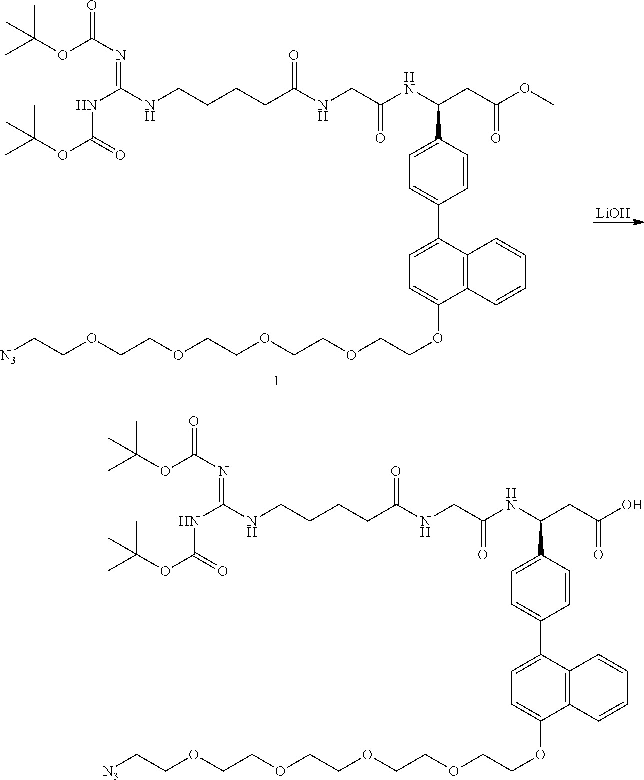

To a solution of compound 1 (120 mg, 0.124 mmol, 1.0 equiv.) in THE (2 mL) and water (2 mL) was added lithium hydroxide (9 mg, 0.373 mmol, 3.0 equiv.) at room temperature. The reaction was kept at room temperature for 2 hrs. The reaction was quenched with HCl (6.0N) and the pH was adjusted to 4.0. The mixture was extracted with ethyl acetate (3×5 mL). The organic phase was combined, dried over anhydrous Na2SO4, and concentrated. The product was used directly without further purification. LC-MS: calculated [M+H]+ 951.47, found 951.47.

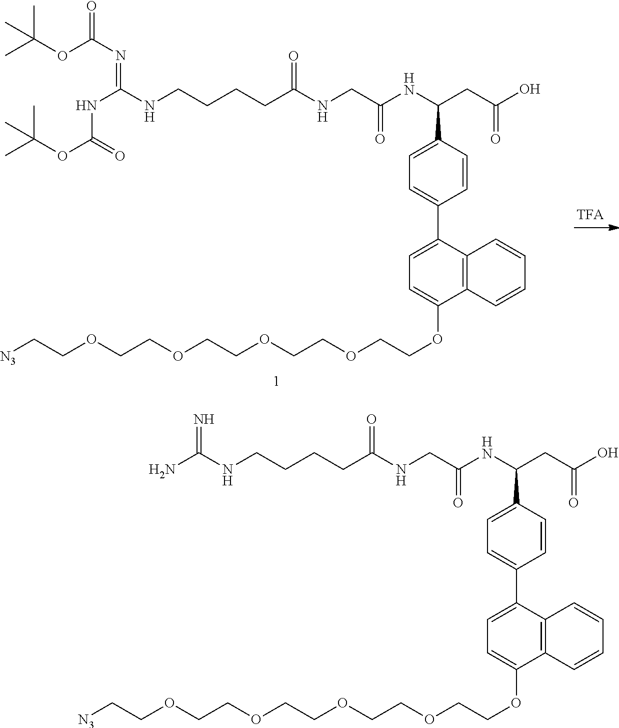 [SMILES: CC(C)(C)OC(=O)/N=C(/NCCCCC(=O)NCC(=O)N[C@@H](CC(=O)O)c1ccc(-c2ccc(OCCOCCOCCOCCOCCN=[N+]=[N-])c3ccccc23)cc1)NC(=O)OC(C)(C)C ; [N-]=[N+]=NCCOCCOCCOCCOCCOc1ccc(-c2ccc([C@H](CC(=O)O)NC(=O)CNC(=O)CCCCNC(=N)N)cc2)c2ccccc12]

To a solution of compound 1 (115 mg, 0.135 mmol, 1.0 equiv.) in dichloromethane (1 mL) was added trifluoroacetic acid (1 mL) at room temperature. The reaction was kept at room temperature for 3 hrs. The solvent was concentrated and the product was used directly without further purification. LC-MS: calculated [M+H]+ 751.37, found 751.43.

### Synthesis of Compound 43p, (S)-3-(2-((S)-2-amino-5-guanidinopentanamido)acetamido)-3-(4-(4-((14-azido-3,6,9,12-tetraoxatetradecyl)oxy)naphthalen-1-yl)phenyl)propanoic acid

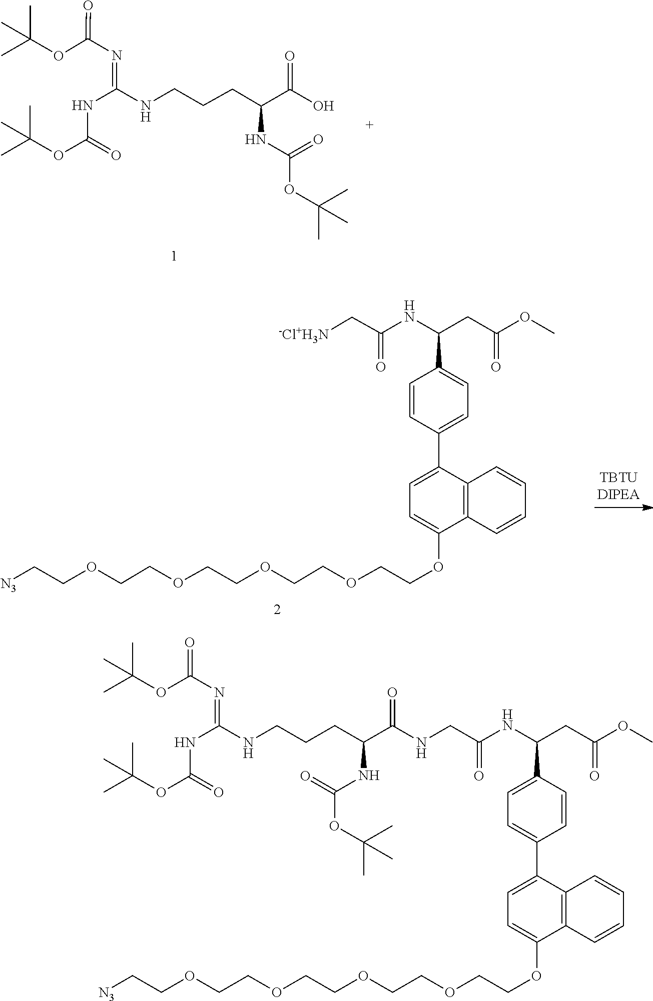

To a solution of compound 1 (72 mg, 0.151 mmol, 1.0 equiv.), compound 2 (105 mg, 0.159 mmol, 1.05 equiv.) and diisopropylethylamine (0.079 mL, 0.588 mmol, 3.0 equiv.) in anhydrous DMF (1 mL) was added TBTU (58 mg, 0.182 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 1080.55, found 1080.57.

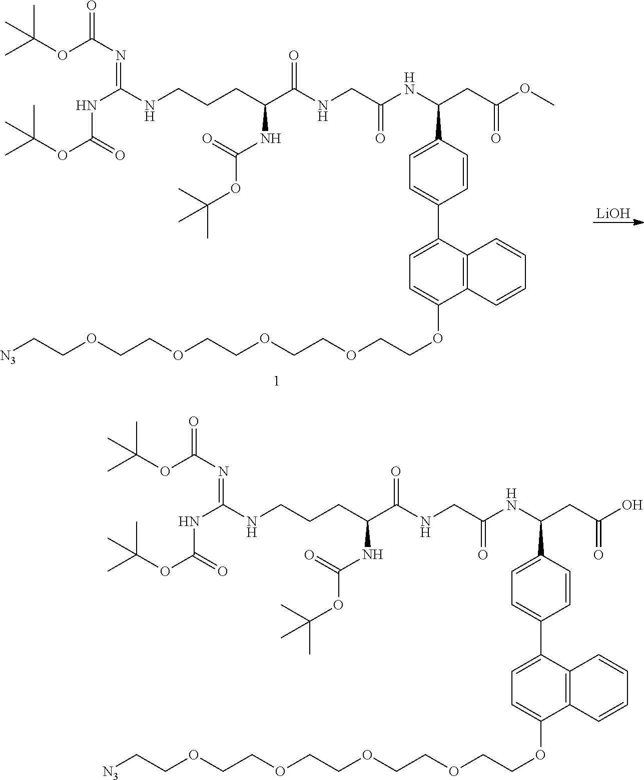

To a solution of compound 1 (100 mg, 0.926 mmol, 1.0 equiv.) in THE (2 mL) and water (2 mL) was added lithium hydroxide (7 mg, 0.277 mmol, 3.0 equiv.) at room temperature. The reaction was kept at room temperature for 2 hrs. The reaction was quenched with HCl (6.0N) and the pH was adjusted to 4.0. The mixture was extracted with ethyl acetate (3×5 mL). The organic phase was combined, dried over anhydrous Na₂SO₄, and concentrated. The product was used directly without further purification. LC-MS: calculated [M+H]+ 1066.54, found 1067.01.

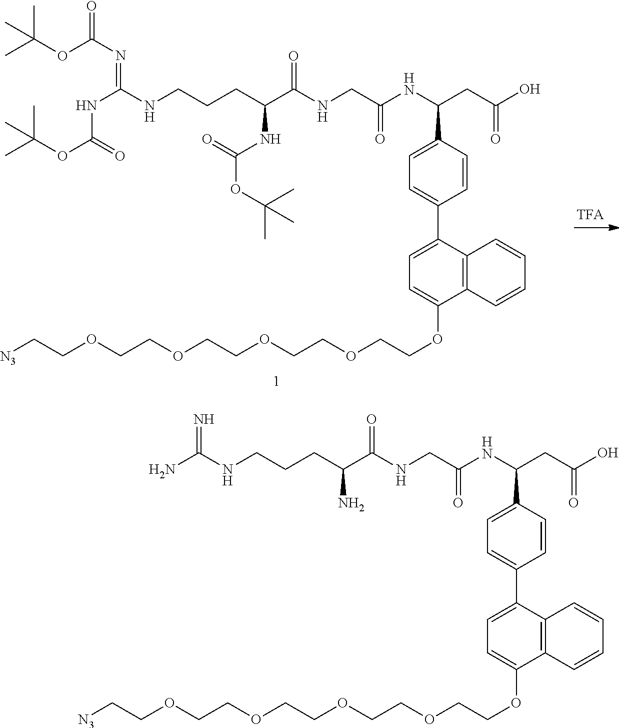 [SMILES: CC(C)(C)OC(=O)/N=C(/NCCC[C@H](NC(=O)OC(C)(C)C)C(=O)NCC(=O)N[C@@H](CC(=O)O)c1ccc(-c2ccc(OCCOCCOCCOCCOCCN=[N+]=[N-])c3ccccc23)cc1)NC(=O)OC(C)(C)C ; [N-]=[N+]=NCCOCCOCCOCCOCCOc1ccc(-c2ccc([C@H](CC(=O)O)NC(=O)CNC(=O)[C@@H](N)CCCNC(=N)N)cc2)c2ccccc12]

To a solution of compound 1 (100 mg, 0.0938 mmol, 1.0 equiv.) in dichloromethane (1 mL) was added trifluoroacetic acid (1 mL) at room temperature. The reaction was kept at room temperature for 3 hrs. The solvent was concentrated and the product was used directly without further purification. LC-MS: calculated [M+H]+ 766.38, found 766.55.

### Synthesis of Compound 44p, (S)-3-(2-((S)-2-acetamido-5-guanidinopentanamido)acetamido)-3-(4-(4-((14-azido-3,6,9,12-tetraoxatetradecyl)oxy)naphthalen-1-yl)phenyl)propanoic acid

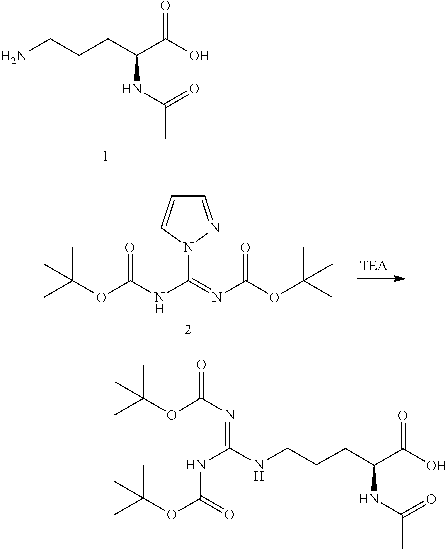

To a solution of compound 1 (500 mg, 2.870 mmol, 1.0 equiv.) and compound 2 (1.081 g, 3.587 mmol, 1.25 equiv.) in anhydrous methanol (10 mL) was added triethylamine (1.20 mL, 8.610 mmol, 3.0 equiv.) at room temperature. The reaction was kept at 40° C. for 2 hrs. The reaction mixture was concentrated and the product was separated by CombiFlash. The product was eluted with 4-6% methanol in dichloromethane. LC-MS: calculated [M+H]+ 417.23, found 417.45.

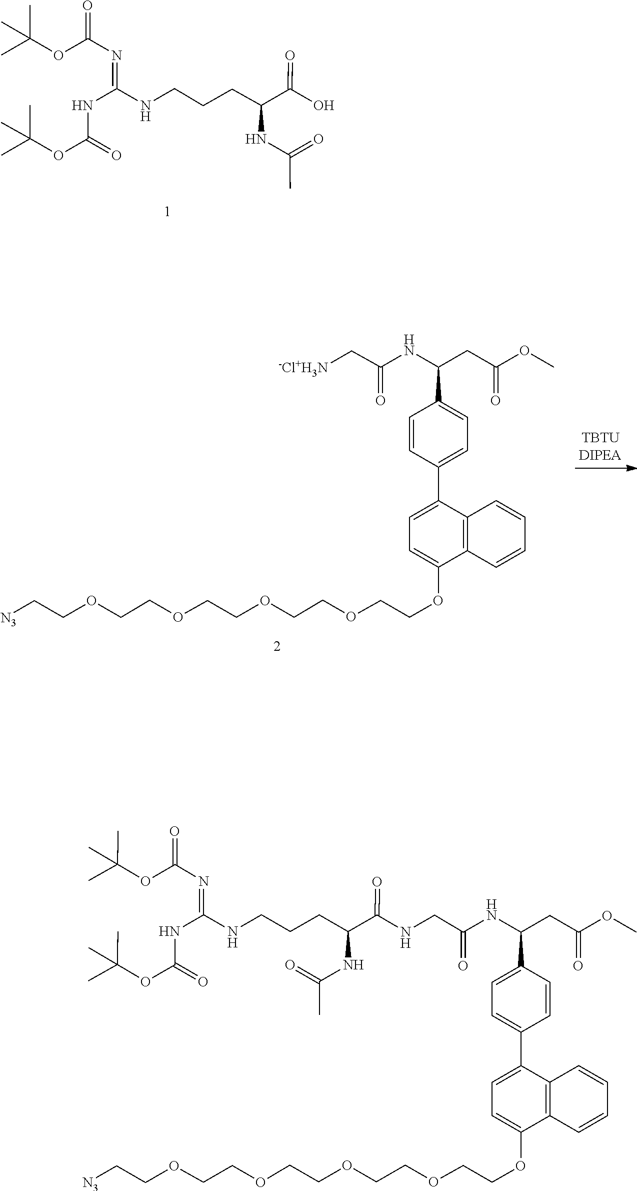

To a solution of compound 1 (66 mg, 0.158 mmol, 1.0 equiv.), compound 2 (109 mg, 0.166 mmol, 1.05 equiv.) and diisopropylethylamine (0.083 mL, 0.475 mmol, 3.0 equiv.) in anhydrous DMF (1 mL) was added TBTU (61 mg, 0.190 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (3×5 mL). The organic phase was combined, dried over anhydrous Na₂SO₄, and concentrated. The product was purified by CombiFlash and was eluted with 2-4% methanol in dichloromethane. LC-MS: calculated [M+H]+ 1022.51, found 1022.36.

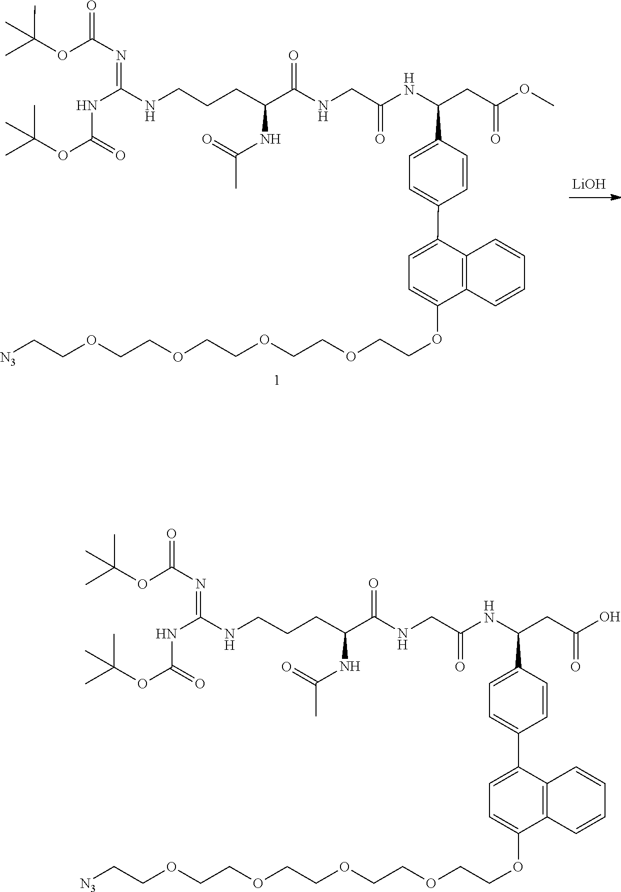 [SMILES: COC(=O)C[C@H](NC(=O)CNC(=O)[C@H](CCCN/C(=N/C(=O)OC(C)(C)C)NC(=O)OC(C)(C)C)NC(C)=O)c1ccc(-c2ccc(OCCOCCOCCOCCOCCN=[N+]=[N-])c3ccccc23)cc1 ; "LiOH" ; CC(=O)N[C@@H](CCCN/C(=N/C(=O)OC(C)(C)C)NC(=O)OC(C)(C)C)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (125 mg, 0.122 mmol, 1.0 equiv.) in THE (2 mL) and water (2 mL) was added lithium hydroxide (9 mg, 0.366 mmol, 3.0 equiv.) at room temperature. The reaction was kept at room temperature for 2 hrs. The reaction was quenched with HCl (6.0N) and the pH was adjusted to 4.0. The mixture was extracted with ethyl acetate (3×5 mL). The organic phase was combined, dried over anhydrous Na₂SO₄, and concentrated. The product was used directly without further purification. LC-MS: calculated [M+H]+ 1008.50, found 1008.79.

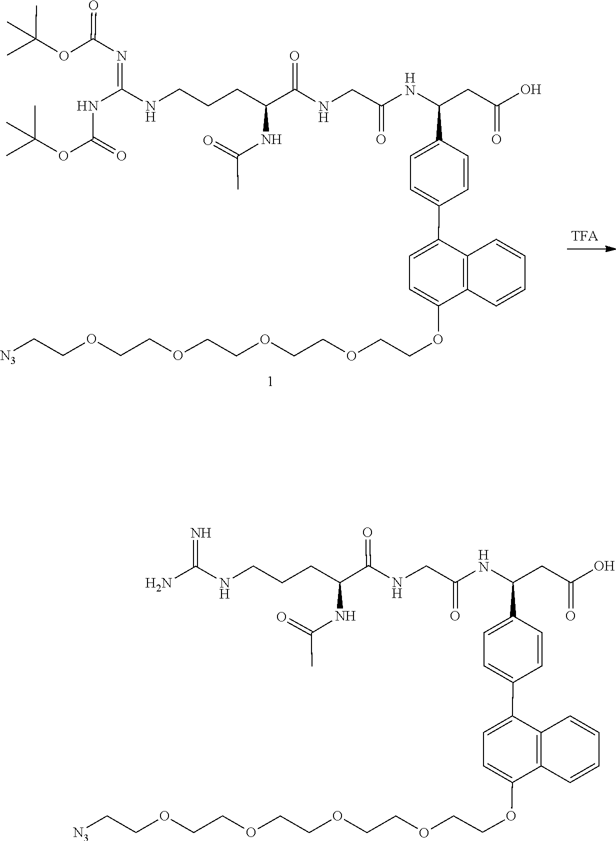 [SMILES: CC(=O)N[C@@H](CCCN/C(=N/C(=O)OC(C)(C)C)NC(=O)OC(C)(C)C)C(=O)NCC(=O)N[C@@H](CC(=O)O)c1ccc(-c2ccc(OCCOCCOCCOCCOCCN=[N+]=[N-])c3ccccc23)cc1 ; CC(=O)N[C@@H](CCCNC(=N)N)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (120 mg, 0.119 mmol, 1.0 equiv.) in dichloromethane (1 mL) was added trifluoroacetic acid (1 mL) at room temperature. The reaction was kept at room temperature for 3 hrs. The solvent was concentrated and the product was used directly without further purification. LC-MS: calculated [M+H]+ 808.39, found 808.33.

### Synthesis of Compound 45p, (S)-3-(4-(4-((14-azido-3,6,9,12-tetraoxatetradecyl)oxy)naphthalen-1-yl)phenyl)-3-(2-(5-((4-methylpyridin-2-yl)amino)pentanamido)acetamido)propanoic acid

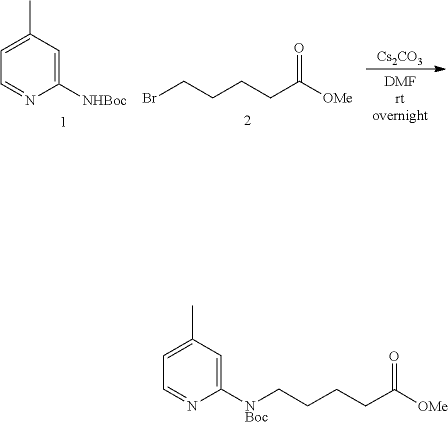

To a solution of compound 1 (0.50 g) in DMF under N₂ (g) at rt was added Cs₂CO₃ (0.94 g). Compound 2 (0.49 g) was then added slowly dropwise. The reaction was stirred overnight. Approx. 50% conversion to desired product by LC-MS was then confirmed. The reaction mixture was quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash using silica gel as the stationary phase with a gradient of hex to EtOAc (0-70%), in which product eluted at 16% B. The product was concentrated under vacuum to provide a clear oil (0.35 g, 45.0% yield). LC-MS: calculated [M+H]+ 323.19 m/z, observed 328.38 m/z.

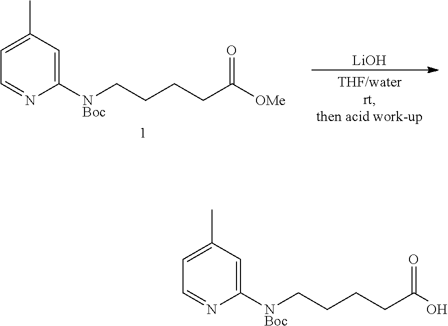 [SMILES: COC(=O)CCCCN(C(=O)OC(C)(C)C)c1cc(C)ccn1 ; "LiOH" ; Cc1ccnc(N(CCCCC(=O)O)C(=O)OC(C)(C)C)c1]

To a solution of compound 1 (0.35 g) in 1:1 THE/water was added LiOH (0.078 g) at rt under normal atmosphere. The reaction was stirred at rt until full conversion was observed by LC-MS. After 1 h, the reaction mixture was acidifed with 6 N HCl to a pH of −3. The product was extracted with EtOAc (3×15 mL). The combined organic phase was dried over Na₂SO₄, filtered, and concentrated, providing a clear, colorless oil (0.32 g, 94.9% yield). No isolation was necessary. LC-MS: calculated [M+H]+ 309.17 m/z, observed 309.24 m/z.

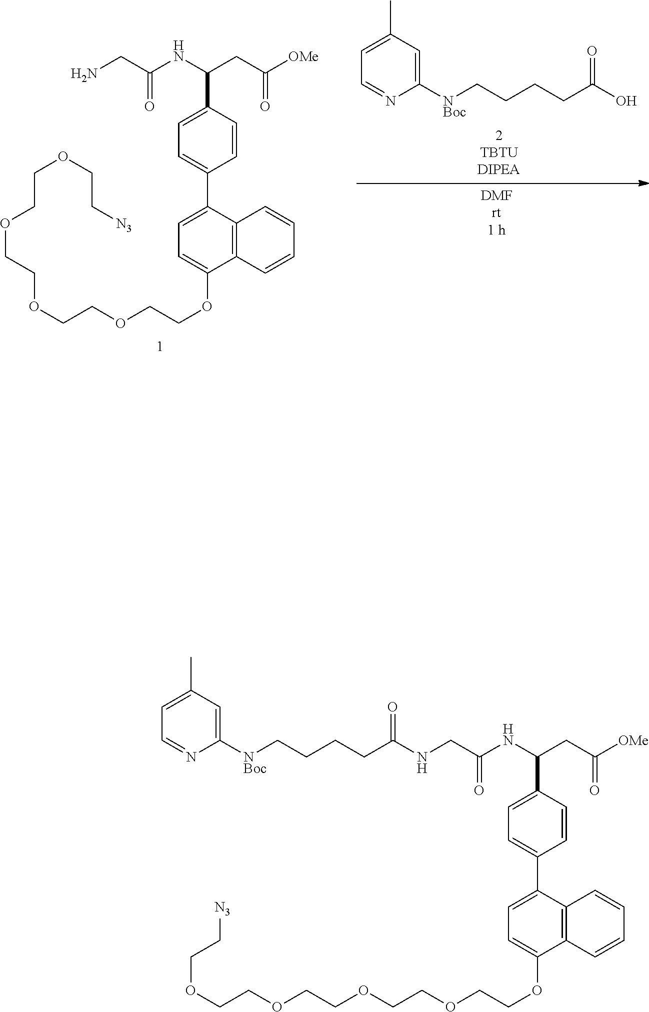 [SMILES: COC(=O)C[C@H](NC(=O)CN)c1ccc(-c2ccc(OCCOCCOCCOCCOCCN=[N+]=[N-])c3ccccc23)cc1 ; Cc1ccnc(N(CCCCC(=O)O)C(=O)OC(C)(C)C)c1 ; COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1]

To a solution of compounds 1 (0.10 g) and 2 (0.049 g) in DMF was added TBTU (0.058 g) and then DIPEA (0.079 mL) under ambient conditions. Reaction was stirred for 1 h until full conversion was observed by LC-MS. The reaction mixture was then quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-70%), in which product eluted at 23% B. The product was concentrated under vacuum to provide a clear colorless oil (0.088 g, yield 63.6%.)

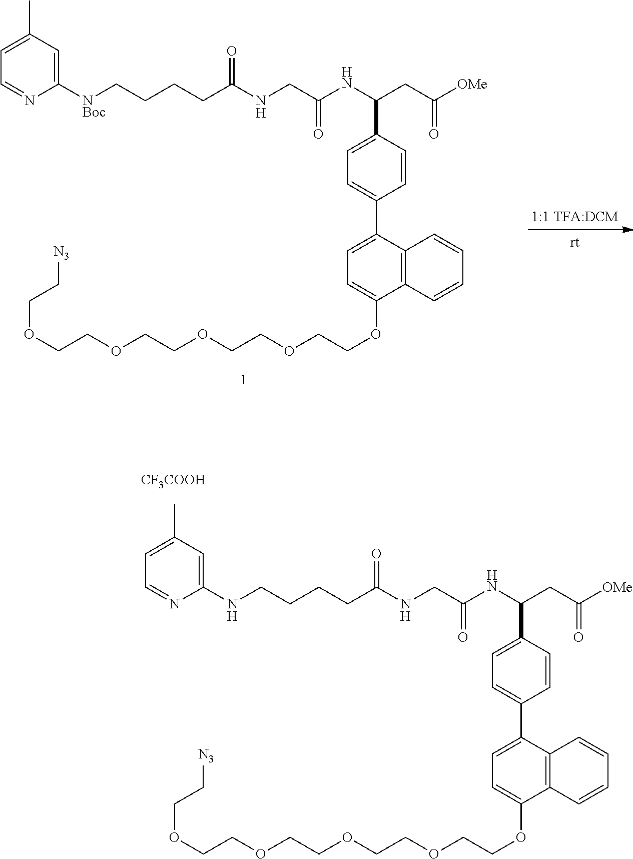 [SMILES: COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1 ; "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1]

To a solution of compound 1 (0.088 g) in DCM was added TFA (0.22 mL) at rt. The reaction was stirred under ambient conditions. Reaction was stirred for 5 h until full conversion was confirmed via LC-MS. The reaction mixture was azeotroped with PhMe and concentrated under vacuum. No isolation was necessary. Concentration provided a clear colorless oil (0.10 g, yield 113%.) LC-MS: calculated [M+H]+ 814.41 m/z, observed 814.63 m/z.

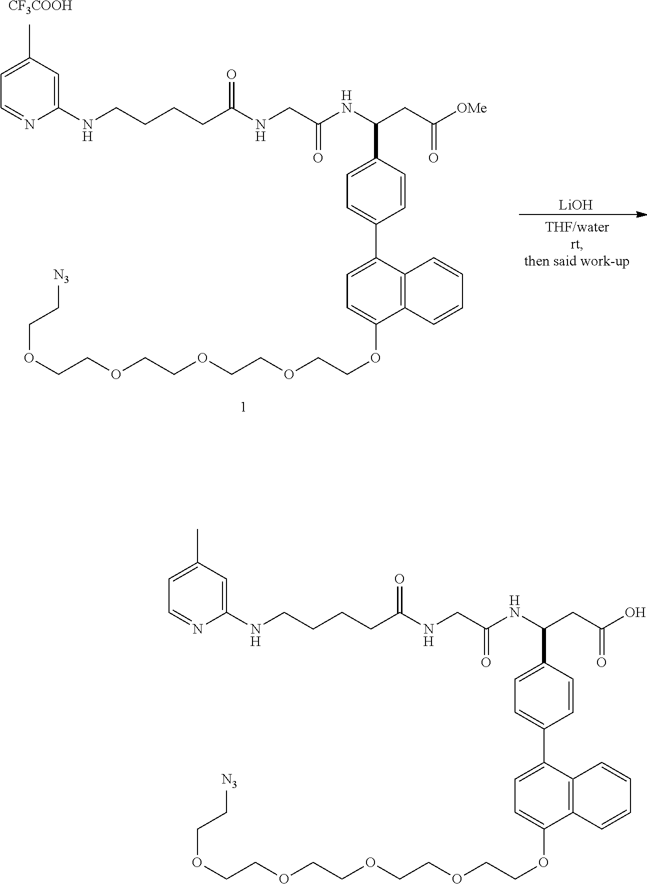 [SMILES: "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1 ; "LiOH" ; Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.10 g) in 1:1 THE/water was added LiOH (0.0078 g) at rt under normal atmosphere. The reaction was stirred at rt until full conversion was observed by LC-MS. After 4 h, the reaction mixture was acidifed with 6 N HCl to a pH of −3. The product was extracted with 20% CF₃CH₂OH/DCM (3×15 mL). The combined organic phase was dried over Na₂SO₄, filtered, and concentrated, providing a light yellow solid (0.104 g, yield 119%.) LC-MS: calculated [M+H]+ 800.39 m/z, observed 800.76 m/z.

### Synthesis of Compound 46p, (S)-3-(4-(4-((14-azido-3,6,9,12-tetraoxatetradecyl)oxy)naphthalen-1-yl)phenyl)-3-(2-(5-((4-methoxypyridin-2-yl)amino)pentanamido)acetamido)propanoic acid

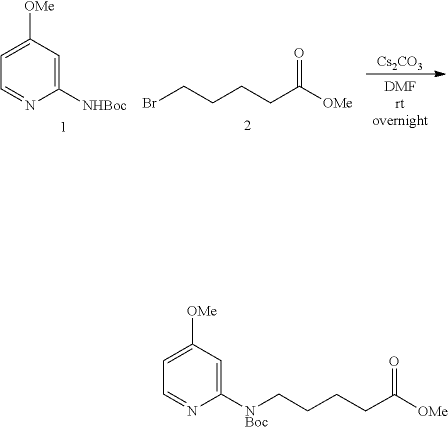

To a solution of compound 1 (0.500 g) in DMF under N₂ (g) at rt was added Cs₂CO₃ (0.872 g). Compound 2 (0.457 g) was then added slowly dropwise. The reaction was stirred overnight. Approx. 50% conversion to desired product by LC-MS was then confirmed. The reaction mixture was quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of hex to EtOAc (0-70%), in which product eluted at 21% B. The product was concentrated under vacuum to provide a clear oil. Yield 0.191 g (25.3%.) LC-MS: calculated [M+H]+ 339.18 m/z, observed 339.31 m/z.

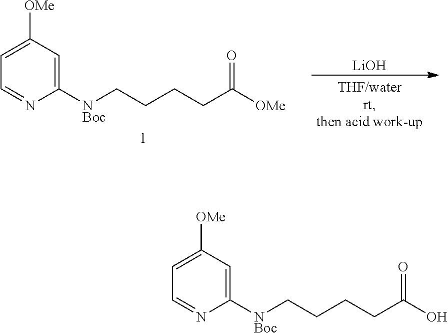 [SMILES: COC(=O)CCCCN(C(=O)OC(C)(C)C)c1cc(OC)ccn1 ; "LiOH" ; COc1ccnc(N(CCCCC(=O)O)C(=O)OC(C)(C)C)c1]

To a solution of compound 1 (0.191 g) in 1:1 THF/water was added LiOH (0.0406 g) at room temperature under normal atmosphere. The reaction was stirred at room temperature until full conversion was observed by LC-MS. After 3 h, the reaction mixture was acidifed with 6 N HCl to a pH of −3. The product was extracted with EtOAc (3×15 mL). The combined organic phase was dried over Na₂SO₄, filtered, and concentrated, providing a clear, colorless oil. Yield: 0.176 g (96.1%). LC-MS: calculated [M+H]+ 325.17 m/z, observed 325.27 m/z.

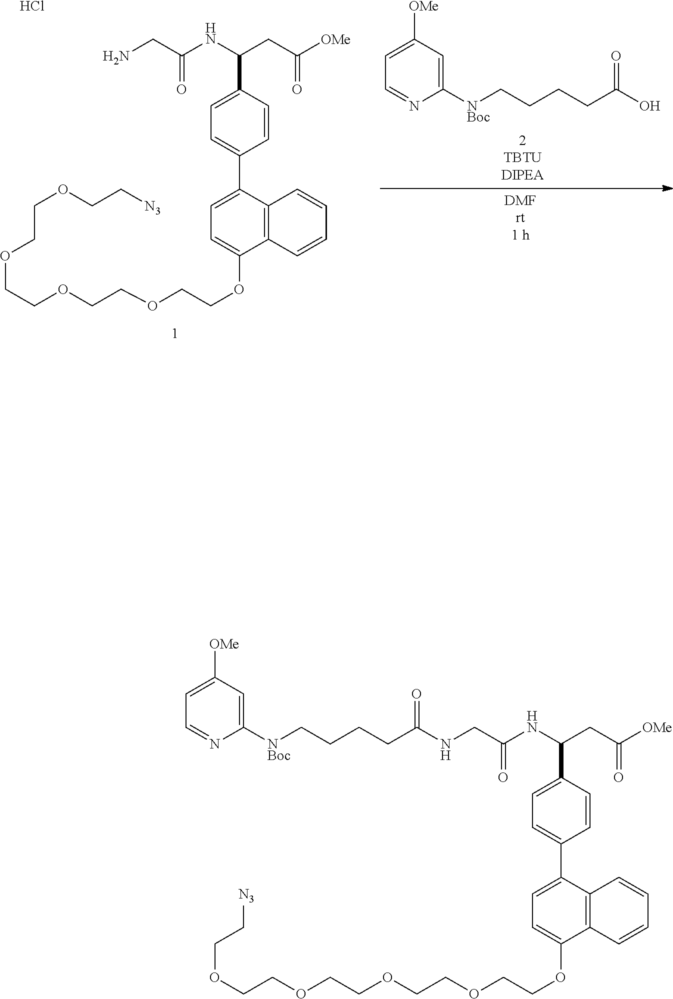 [SMILES: Cl ; COC(=O)C[C@H](NC(=O)CN)c1ccc(-c2ccc(OCCOCCOCCOCCOCCN=[N+]=[N-])c3ccccc23)cc1 ; COc1ccnc(N(CCCCC(=O)O)C(=O)OC(C)(C)C)c1 ; COC(=O)C[C@H](NC(=O)CNC(=O)CCCCN(C(=O)OC(C)(C)C)c1cc(OC)ccn1)c1ccc(-c2ccc(OCCOCCOCCOCCOCCN=[N+]=[N-])c3ccccc23)cc1]

To a solution of compounds 1 (0.100 g) and 2 (0.0516 g) in DMF was added TBTU (0.0584 g) and then DIPEA (0.0587 g) under ambient conditions. The reaction was stirred for 1 h until full conversion was observed by LC-MS. The reaction mixture was then quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-75%), in which product eluted at 25% B. The product was concentrated under vacuum to provide a clear colorless oil. Yield: 0.108 g (76.7%.) LC-MS: calculated [M+H]+ 930.45 m/z, observed 930.94 m/z.

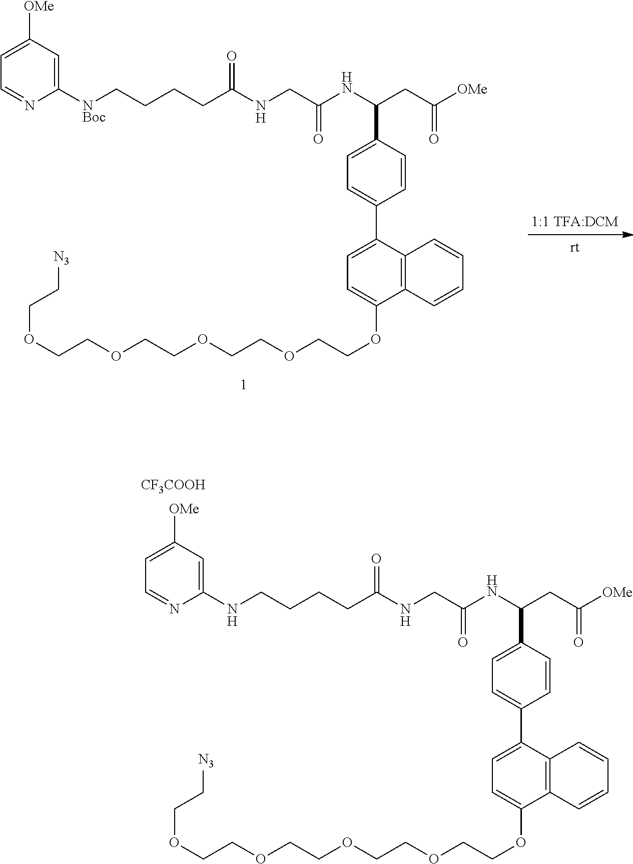 [SMILES: COC(=O)C[C@H](NC(=O)CNC(=O)CCCCN(C(=O)OC(C)(C)C)c1cc(OC)ccn1)c1ccc(-c2ccc(OCCOCCOCCOCCOCCN=[N+]=[N-])c3ccccc23)cc1 ; "CF3COOH" ; COC(=O)C[C@H](NC(=O)CNC(=O)CCCCNc1cc(OC)ccn1)c1ccc(-c2ccc(OCCOCCOCCOCCOCCN=[N+]=[N-])c3ccccc23)cc1]

To a solution of compound 1 (0.180 g) in DCM was added TFA (0.3972 g) at room temperature. The reaction was stirred under ambient conditions. Reaction was stirred for 5 h until full conversion was confirmed via LC-MS. The reaction mixture was azeotroped with PhMe and concentrated under vacuum. No isolation was necessary. Concentration provided a clear colorless oil. Yield 0.121 g (110%). LC-MS: calculated [M+H]+ 830.40 m/z, observed 830.65 m/z.

### CF₃COOH

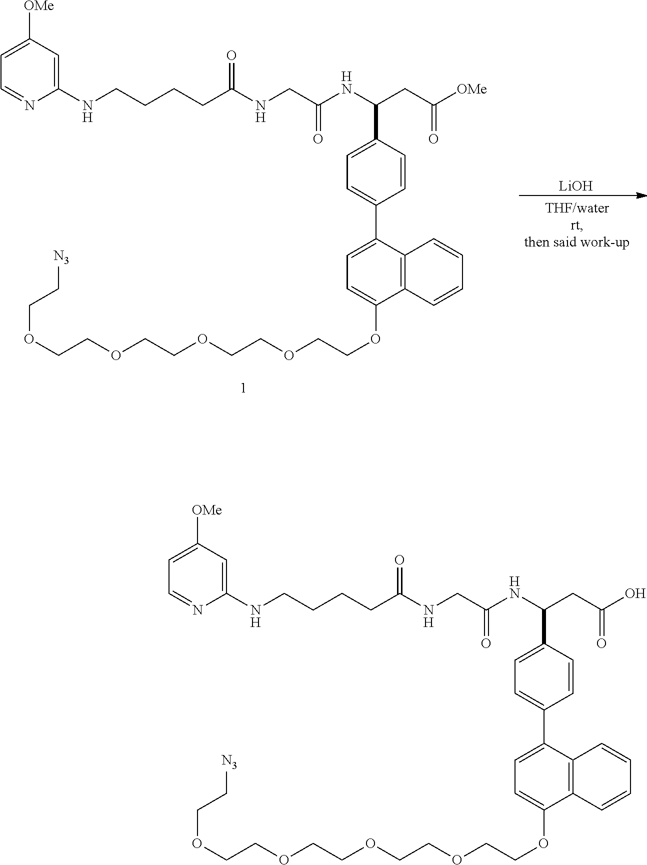 [SMILES: COC(=O)C[C@H](NC(=O)CNC(=O)CCCCNc1cc(OC)ccn1)c1ccc(-c2ccc(OCCOCCOCCOCCOCCN=[N+]=[N-])c3ccccc23)cc1 ; "LiOH" ; COc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.121 g) in 1:1 THF/water was added LiOH (0.0092 g) at rt under normal atmosphere. The reaction was stirred at room temperature until full conversion was observed by LC-MS. After 4 h, the reaction mixture was acidifed with 6 N HCl to a pH of −3. The product was extracted with 20% CF₃CH₂OH/DCM (3×15 mL). The combined organic phase was dried over Na₂SO₄, filtered, and concentrated, providing a cream white solid. Yield 0.122 g (117%.) LC-MS: calculated [M+H]+ 816.39 m/z, observed 816.52 m/z.

### Synthesis of Compound 47p, (S)-3-(2-((S)-2-amino-5-ureidopentanamido)acetamido)-3-(4-(4-((14-azido-3,6,9,12-tetraoxatetradecyl)oxy)naphthalen-1-yl)phenyl)propanoic acid

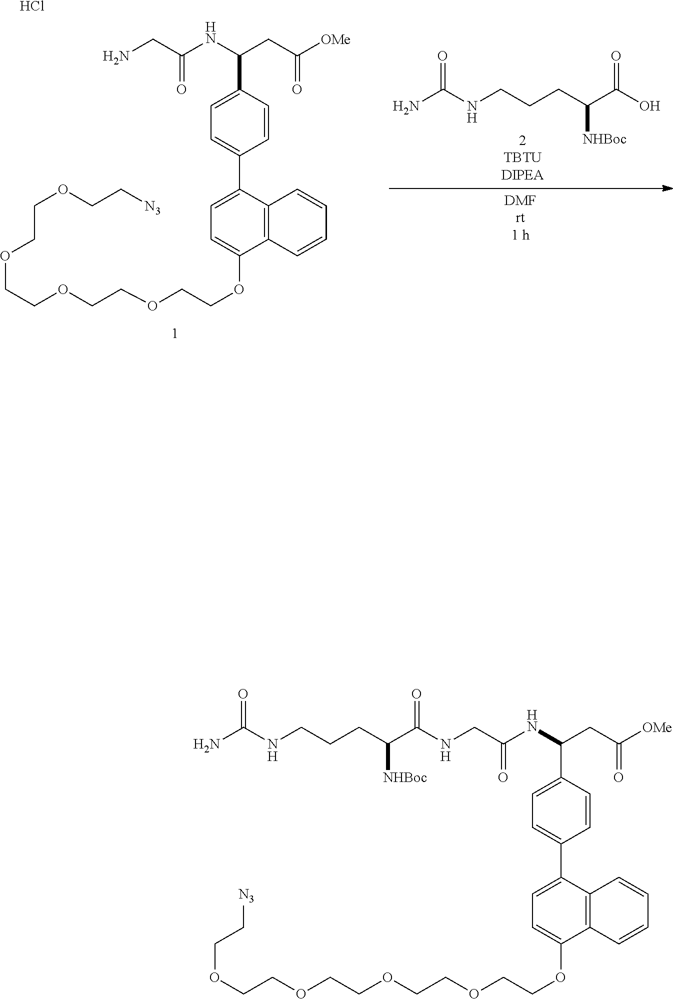

To a solution of compounds 1 (0.144 g) and 2 (0.0601 g) in DMF was added TBTU (0.0840 g) and then DIPEA (0.114 mL) under ambient conditions. The reaction was stirred for 1 h until full conversion was observed by LC-MS. The reaction mixture was then quenched with NaHCO₃(10 mL). The product was extracted with 20% CF₃CH₂OH/DCM (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-100%), in which product eluted at 47% B. The product was concentrated under vacuum to provide a clear colorless oil. Yield 0.149 g (77.7%.) LC-MS: calculated [M+H]+ 881.43 m/z, observed 881.61 m/z.

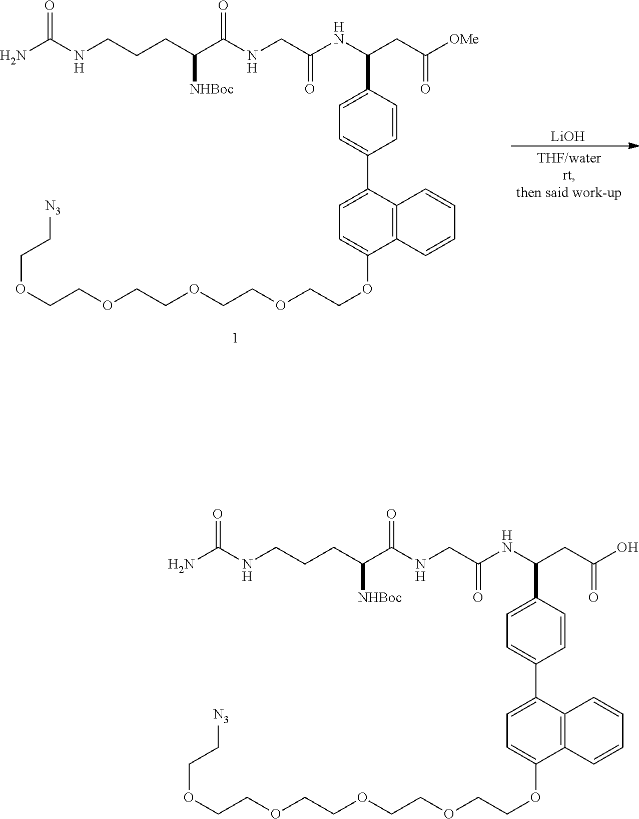 [SMILES: COC(=O)C[C@H](NC(=O)CNC(=O)[C@H](CCCNC(N)=O)NC(=O)OC(C)(C)C)c1ccc(-c2ccc(OCCOCCOCCOCCOCCN=[N+]=[N-])c3ccccc23)cc1 ; "LiOH" ; CC(C)(C)OC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (0.149 g) in 1:1 THF/water was added LiOH (0.0122 g) at rt under normal atmosphere. The reaction was stirred at room temperature until full conversion was observed by LC-MS. After 1 h, the reaction mixture was acidifed with 6 N HCl to a pH of −3. The product was extracted with 20% CF₃CH₂OH/DCM (5×10 mL). The combined organic phase was dried over Na₂SO₄, filtered, and concentrated, providing a white solid. Yield: 0.148 g (100%) LC-MS: calculated [M+H]+ 867.42 m/z, observed 867.83 m/z.

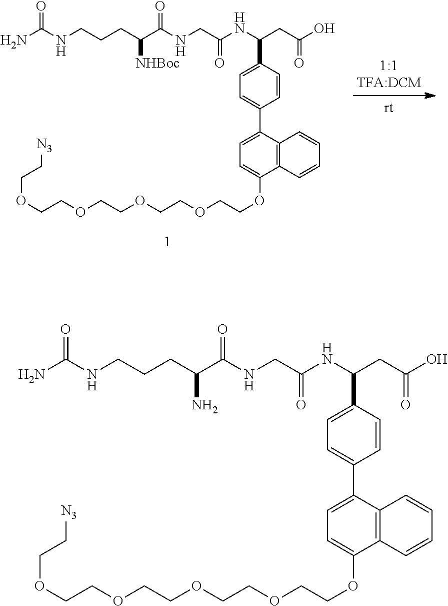 [SMILES: CC(C)(C)OC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1 ; [N-]=[N+]=NCCOCCOCCOCCOCCOc1ccc(-c2ccc([C@H](CC(=O)O)NC(=O)CNC(=O)[C@@H](N)CCCNC(N)=O)cc2)c2ccccc12]

To a solution of compound 1 (0.148 g) in DCM was added TFA (0.392 mL) at room temperature. The reaction was stirred under ambient conditions. The reaction was stirred for 1 h until full conversion was confirmed via LC-MS. The reaction mixture was azeotroped with PhMe and concentrated under vacuum.

The mixture was found to be messy due to incomplete saponification in previous step, so the mixture was resubjected to basic conditions (LiOH, THE/water, rt) for 1 h. Upon confirmation of full conversion, mixture was acidified with 6 N HCl to a pH of −3, and product was extracted with EtOAc and then 20% CF₃CH₂OH/DCM and then dried over Na₂SO₄, filtered, and concentrated. Concentration provided a white solid. Yield 0.162 g (124%.) LC-MS: calculated [M+H]+ 767.36 m/z, observed 767.55 m/z.

### Synthesis of Compound 48p, (S)-3-(2-((S)-2-acetamido-5-ureidopentanamido)acetamido)-3-(4-(4-((14-azido-3,6,9,12-tetraoxatetradecyl)oxy)naphthalen-1-yl)phenyl)propanoic acid

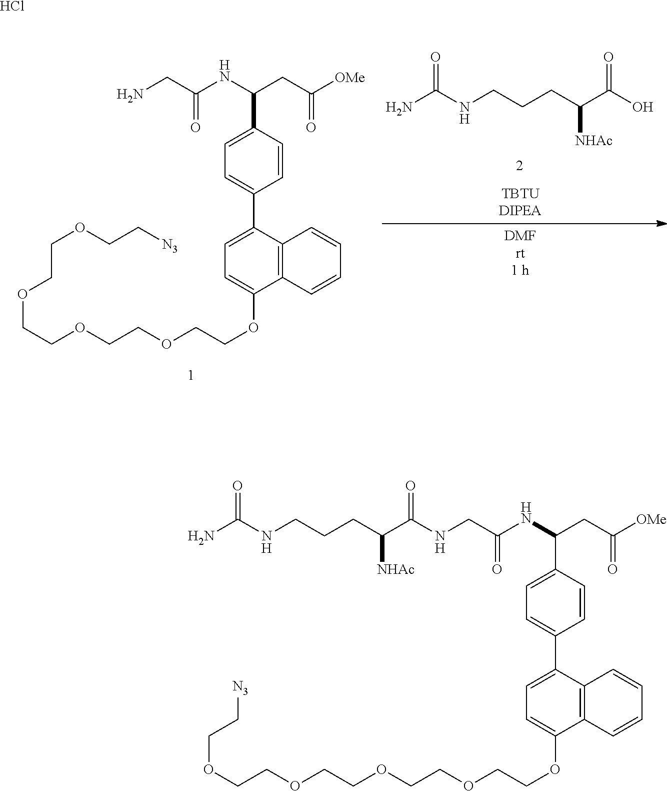

To a solution of compounds 1 (0.183 g) and 2 (0.0602 g) in DMF was added TBTU (0.107 g) and then DIPEA (0.145 mL) under ambient conditions. The reaction was stirred for 1 h until full conversion was observed by LC-MS. The reaction mixture was then quenched with NaHCO₃(10 mL). The product was extracted with EtOAc and then 20% CF₃CH₂OH/DCM (3×15 mL). The combined organic phase was dried over Na₂SO₄, filtered, and concentrated. The mixture was then azeotroped with PhMe. The residue was purified by CombiFlash® using silica gel as the stationary phase with a gradient of DCM to 20% MeOH in DCM (0-100%), in which product eluted at 65% B. An impurity had eluted with product, so the residue was reisolated via a gradient of DCM to 20% MeOH in DCM (0-80%), in which product eluted from 0-70% B; however, the impurity was not able to be isolated. The product was concentrated under vacuum to provide a clear colorless oil. Yield: 0.0378 g (16.6%.) LC-MS: calculated [M+H]+ 823.39 m/z, observed 823.27 m/z.

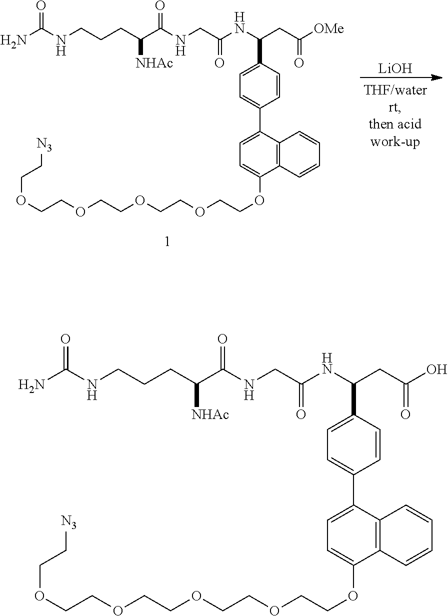 [SMILES: COC(=O)C[C@H](NC(=O)CNC(=O)[C@H](CCCNC(N)=O)NC(C)=O)c1ccc(-c2ccc(OCCOCCOCCOCCOCCN=[N+]=[N-])c3ccccc23)cc1 ; "LiOH" ; CC(=O)N[C@@H](CCCNC(N)=O)C(=O)NCC(=O)N[C@@H](CC(=O)O)c1ccc(-c2ccc(OCCOCCOCCOCCOCCN=[N+]=[N-])c3ccccc23)cc1]

To a solution of compound 1 (0.0378 g) in 1:1 THF/water was added LiOH (0.0033) at room temperature under normal atmosphere. The reaction was stirred at room temperature until full conversion was observed by LC-MS. After 1 h, the reaction mixture was acidifed with 6 N HCl to a pH of −3. The product was extracted with 20% CF₃CH₂OH/DCM (5×10 mL). The combined organic phase was dried over Na₂SO₄, filtered, and concentrated, providing a yellow solid. Isolation was found to be necessary. The mixture was solvated in 1 mL of DMF, and product was isolated via reverse-phase HPLC to provide a clear and colorless residue. Yield: 0.088 g (237%.) LC-MS: calculated [M+H]+ 809.38 m/z, observed 809.68 m/z.

### Synthesis of Compound 49p, (S)-3-(2-((S)-2-amino-5-((4-methylpyridin-2-yl)amino)pentanamido)acetamido)-3-(4-(4-((14-azido-3,6,9,12-tetraoxatetradecyl)oxy)naphthalen-1-yl)phenyl)propanoic acid

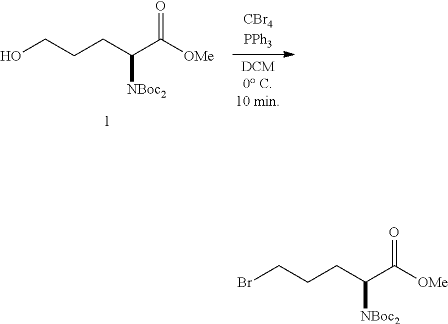

To a solution of compound 1 (0.620 g) in DCM, under N₂(g) at 0° C. in ice-water bath, was added CBr₄ (0.680 g); the mixture was stirred on ice for 15 min. Then PPh₃ (0.538 g) was added, and reaction was stirred for 10 min., after which full conversion to the desired product was observed by LC-MS; a clean mixture of desired pdt, O=PPh3, and other PPh3-based by-product was observed. The reaction mixture was then quenched with NaHCO₃(10 mL). The product was extracted with DCM (3×10 mL) and then washed with brine (10 mL). The combined organic phase was dried over Na₂SO₄, filtered, and concentrated. The residue was purified by CombiFlash® using silica gel as the stationary phase with a gradient of hex to EtOAc (0-30%), in which product eluted at 8.5% B. The product was concentrated under vacuum, providing a clear colorless oil. Yield: 0.597 g (81.6%.) LC-MS: calculated [M+H]+ 410.11 m/z, observed 410.43 m/z.

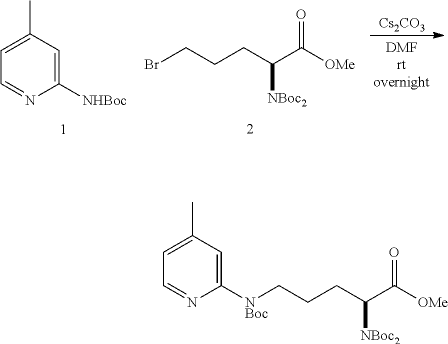

To a solution of compounds 1 (0.134 g) and 2 (0.238 g) in DMF at room temperature was added Cs₂CO₃ (0.315 g). The reaction was stirred overnight. Approx. 50% conversion to desired product by LC-MS was then confirmed. The reaction mixture was quenched with NaHCO₃(10 mL). The product was extracted with DCM (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of hexanes to EtOAc (0-70%), in which product eluted at 15% B. The product was concentrated under vacuum to provide a clear oil. Yield: 0.196 g (56.6%.) LC-MS: calculated [M+H]+ 538.31 m/z, observed 538.44 m/z.

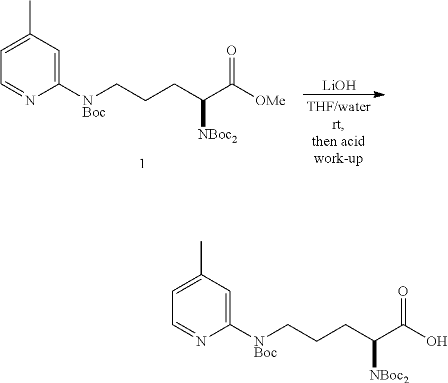

To a solution of compound 1 (0.196 g) in 1:1 THE/water was added LiOH (0.262 g) at room temperature under normal atmosphere. The reaction was stirred at room temperature until full conversion was observed by LC-MS. After 7 h with low conversion, the reaction mixture was heated to 30° C. and stirred overnight. Once full conversion was confirmed by LC-MS, the reaction mixture was slowly acidifed with 6 N HCl to a pH of −5. The product was extracted with EtOAc (3×15 mL). The combined organic phase was dried over Na₂SO₄, filtered, and concentrated, providing a clear, colorless oil. Yield: 0.186 g (97.1%.) LC-MS: calculated [M+H]+ 524.29 m/z, observed 524.67 m/z.

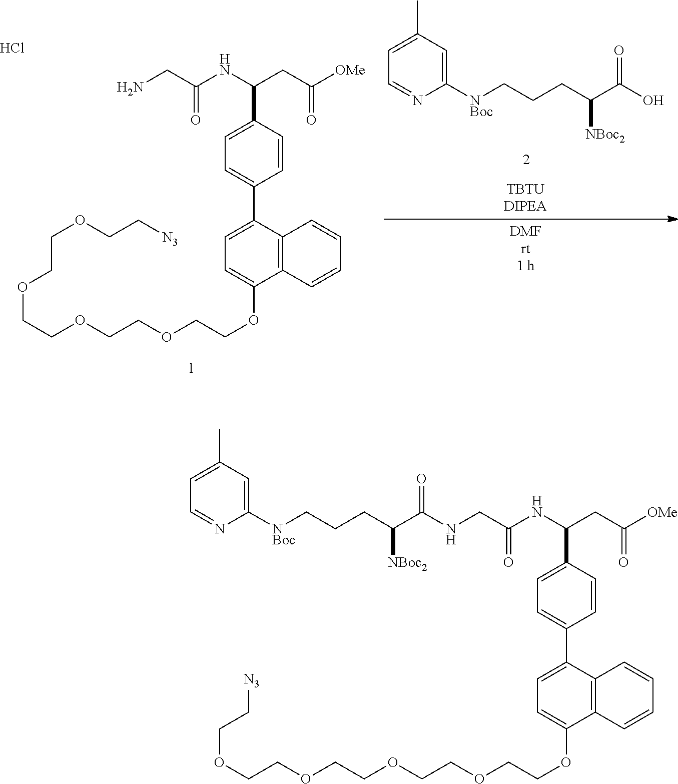

To a solution of compounds 1 (0.246 g) and 2 (0.185 g) in DMF was added TBTU (0.1436 g) and then DIPEA (0.195 mL) under ambient conditions. The reaction was stirred for 1 h until full conversion was observed by LC-MS. The reaction mixture was then quenched with NaHCO₃(10 mL). The product was extracted with EtOAc and then 20% CF₃CH₂OH/DCM (3×15 mL), and then washed with water (3×10 mL). The combined organic phase was dried over Na₂SO₄, filtered, and concentrated. The residue was purified by CombiFlash® using silica gel as the stationary phase with a gradient of DCM to 20% MeOH in DCM (0-60%), in which product eluted at 13-26% B. The product was concentrated under vacuum to provide a clear colorless oil. Product appears to contain a mixture of desired product and mono-Boc-deprotected product. Yield: 0.212 g (50.3%.) LC-MS: calculated [M+H]+ 1129.57 m/z, observed 1130.02 m/z.

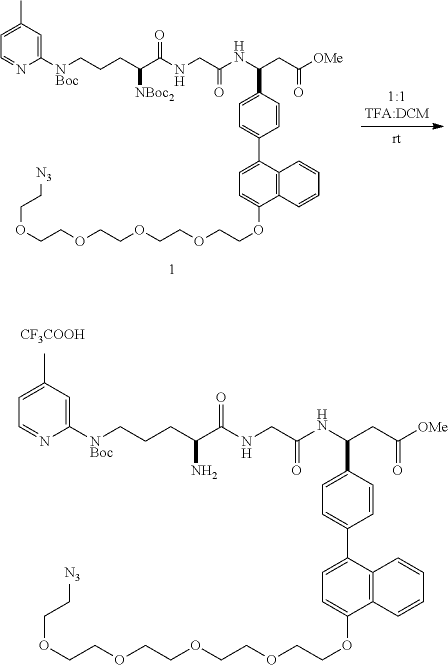

To a solution of compound 1 (0.0636 g) in DCM was added TFA (0.129 mL) at room temperature. The reaction was stirred under ambient conditions. After 6 h, full conversion was confirmed via LC-MS. The reaction mixture was azeotroped with PhMe and concentrated under vacuum. No isolation was necessary. Concentration provided a sticky yellow residue. Yield: 0.0686 g (129%.) LC-MS: calculated [M+H]+ 829.42 m/z, observed 829.57 m/z.

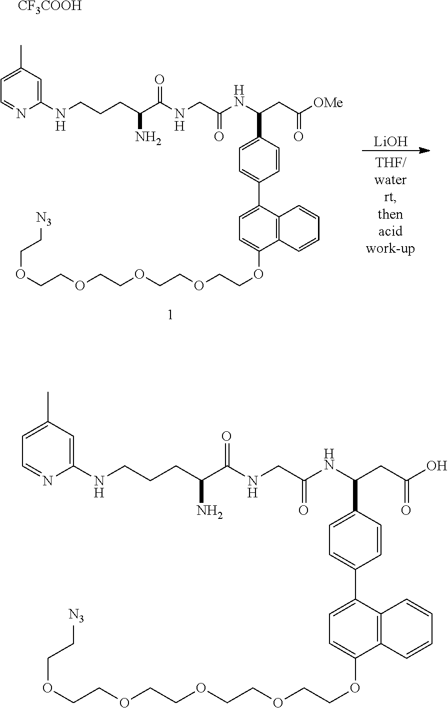 [SMILES: "CF3COOH" ; COC(=O)C[C@H](NC(=O)CNC(=O)[C@@H](N)CCCNc1cc(C)ccn1)c1ccc(-c2ccc(OCCOCCOCCOCCOCCN=[N+]=[N-])c3ccccc23)cc1 ; "LiOH" ; Cc1ccnc(NCCC[C@H](N)C(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.0250 g) in 1:1 DMF/water was added LiOH (0.0019 g) at room temperature under normal atmosphere. The reaction was stirred at room temperature for 3 h, 40° C. for 3-4 h, and then room temperature overnight. The following day, the reaction was stirred at 40° C. until full conversion was observed by LC-MS. The reaction mixture was then acidifed with 6 N HCl to a pH of −7. The mixture was concentrated to 2 mL of solution and isolated via reverse-phase HPLC. The product was then concentrated, providing a clear and colorless residue. Yield 0.0111 g (51.4%.) LC-MS: calculated [M+H]+ 815.40 m/z, observed 815.98 m/z.

### Synthesis of Compound 50p, (S)-3-(2-((S)-2-acetamido-5-((4-methylpyridin-2-yl)amino)pentanamido)acetamido)-3-(4-(4-((14-azido-3,6,9,12-tetraoxatetradecyl)oxy)naphthalen-1-yl)phenyl)propanoic acid

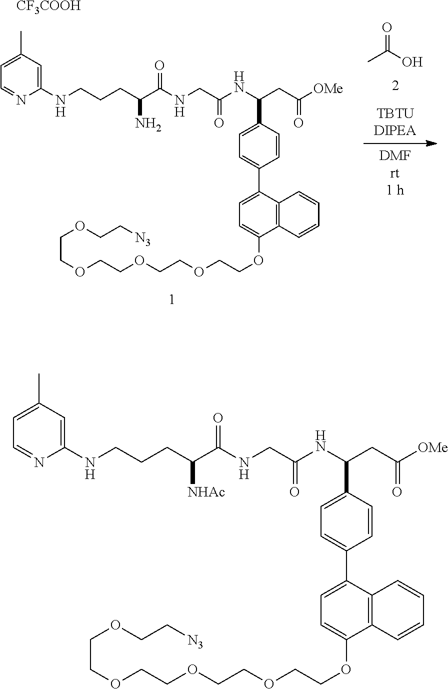

To a solution of compounds 1 (0.0350 g) and 2 (0.0022 g) in DMF was added TBTU (0.0143 g) and then DIPEA (0.019 mL) under ambient conditions. The reaction was stirred for 2 h until full conversion was observed by LC-MS. The reaction mixture was then quenched with NaHCO₃(10 mL). The product was extracted with EtOAc and then 20% CF₃CH₂OH/DCM (3×15 mL) and then washed with water (3×10 mL). The combined organic phase was dried over Na₂SO₄, filtered, and concentrated. The residue was purified by CombiFlash® using silica gel as the stationary phase with a gradient of DCM to 20% MeOH in DCM (0-80%), in which product eluted at 47% B. The product was concentrated under vacuum to provide a clear colorless residue. Yield: 0.0126 g (39.0%.) LC-MS: calculated [M+H]+ 871.43 m/z, observed 872.33 m/z.

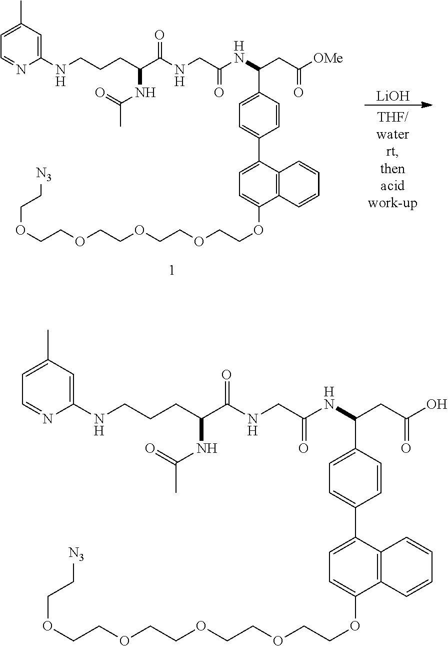

To a solution of compound 1 (0.0126 g) in 1:1 THE/water was added LiOH (0.0010 g) at room temperature under normal atmosphere. The reaction was stirred at room temperature until full conversion was observed by LC-MS. After 1 h, the reaction mixture was acidifed with 6 N HCl to a pH of −4. The product was extracted with EtOAc and then 20% CF₃CH₂OH/DCM (3×10 mL) and washed with water (3×5 mL) and brine (1×5 mL). The combined organic phase was dried over Na₂SO₄, filtered, and concentrated, providing a honey-colored residue. No isolation was necessary. Yield: 0.166 g (134%.) LC-MS: calculated [M+H]+ 857.41 m/z, observed 857.21 m/z.

### Synthesis of Compound 51p, (S)-3-(4-(4-((17-azido-3,6,9,12,15-pentaoxaheptadecyl)carbamoyl)naphthalen-1-yl)phenyl)-3-(2-(4-((4-methylpyridin-2-yl)amino)butanamido)acetamido)propanoic acid

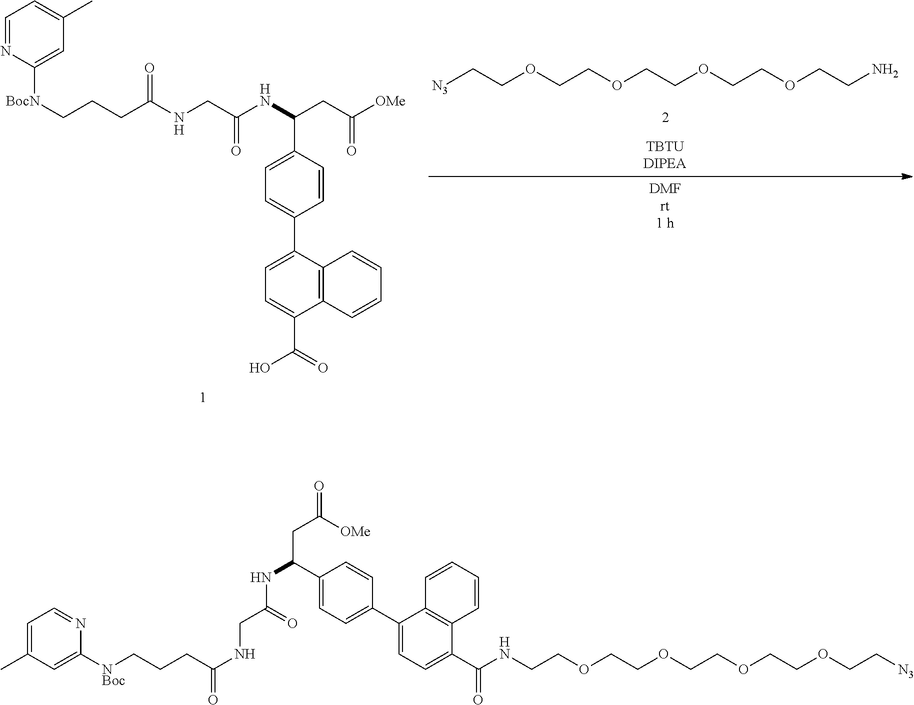 [SMILES: COC(=O)C[C@H](NC(=O)CNC(=O)CCCN(C(=O)OC(C)(C)C)c1cc(C)ccn1)c1ccc(-c2ccc(C(=O)O)c3ccccc23)cc1 ; [N-]=[N+]=NCCOCCOCCOCCOCCN ; COC(=O)C[C@H](NC(=O)CNC(=O)CCCN(C(=O)OC(C)(C)C)c1cc(C)ccn1)c1ccc(-c2ccc(C(=O)NCCOCCOCCOCCOCCN=[N+]=[N-])c3ccccc23)cc1]

To a solution of compounds 1 (0.0250 g) and 2 (0.0118 g) in DMF was added TBTU (0.0141 g) and then DIPEA (0.019 mL) under ambient conditions. The reaction was stirred for 3 h until full conversion was observed by LC-MS. The reaction mixture was then quenched with NaHCO₃(10 mL). The product was extracted with EtOAc and then 20% CF₃CH₂OH/DCM (3×15 mL) and then washed with water (3×10 mL). The combined organic phase was dried over Na₂SO₄, filtered, and concentrated. The residue was purified by CombiFlash® using silica gel as the stationary phase with a gradient of DCM to 20% MeOH in DCM (0-80%), in which product eluted at 36% B. The product was concentrated under vacuum to provide a clear colorless oil. Yield: 0.0233 g (65.5%.) LC-MS: calculated [M+H]+ 971.48 m/z, observed 971.99 m/z.

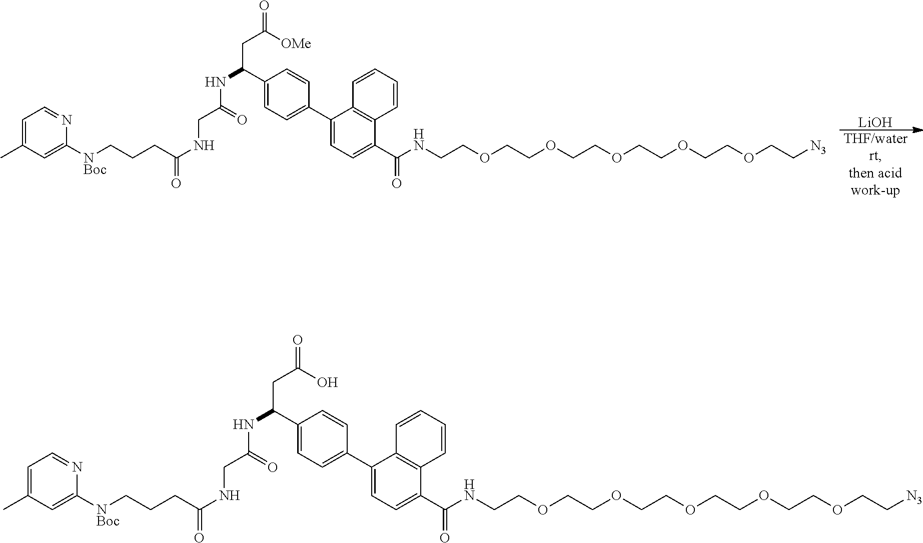

To a solution of compound 1 (0.0233 g) in 1:1 DMF/water was added LiOH (0.0017 g) at rt under normal atmosphere. The reaction was stirred at room temperature for 1 h until full conversion was observed by LC-MS. The reaction mixture was then acidifed with 6 N HCl to a pH of −4, extracted with EtOAc and then 20% CF₃CH₂OH/DCM (5×8 mL), and then washed with water and brine (3×8 mL). The product was then concentrated, providing a white solid. Yield: 0.0281 g (123%.) LC-MS: calculated [M+H]+ 957.46 m/z, observed 957.86 m/z.

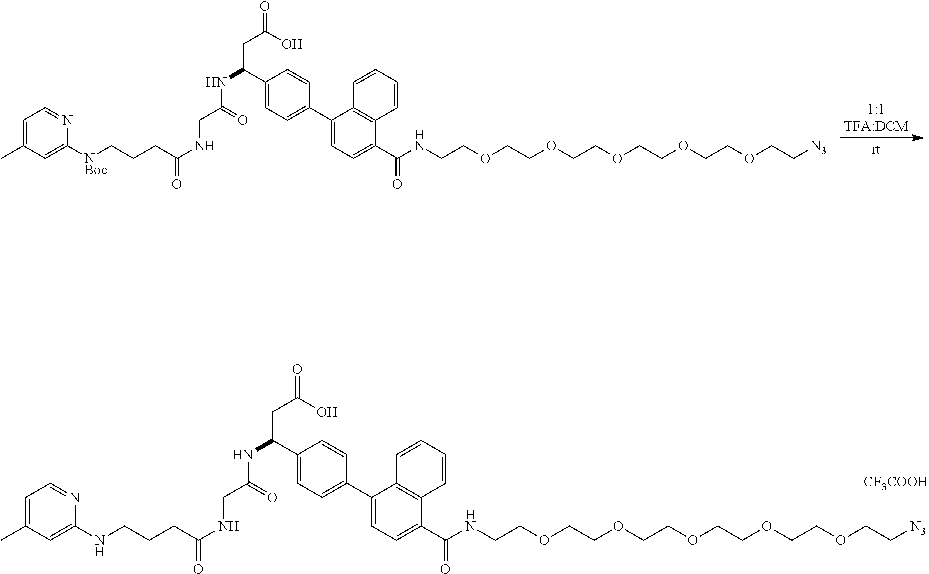

To a solution of compound 1(0.0281 g) in DCM was added TFA (0.067 mL) at rt. The reaction was stirred under ambient conditions. After 2 h, full conversion was confirmed via LC-MS. The reaction mixture was azeotroped with PhMe and concentrated under vacuum. No isolation was necessary. Concentration provided a clear and colorless residue. Yield: 0.0415 (146%.) LC-MS: calculated [M+H]+ 857.41 m/z, observed 857.39 m/z.

### Synthesis of Compound 52p, (S)-3-(4-(4-(((S)-1-azido-22-methyl-19-oxo-3,6,9,12,15-pentaoxa-18-azatricosan-20-yl)carbamoyl)naphthalen-1-yl)phenyl)-3-(2-(4-((4-methylpyridin-2-yl)amino)butanamido)acetamido)propanoic acid

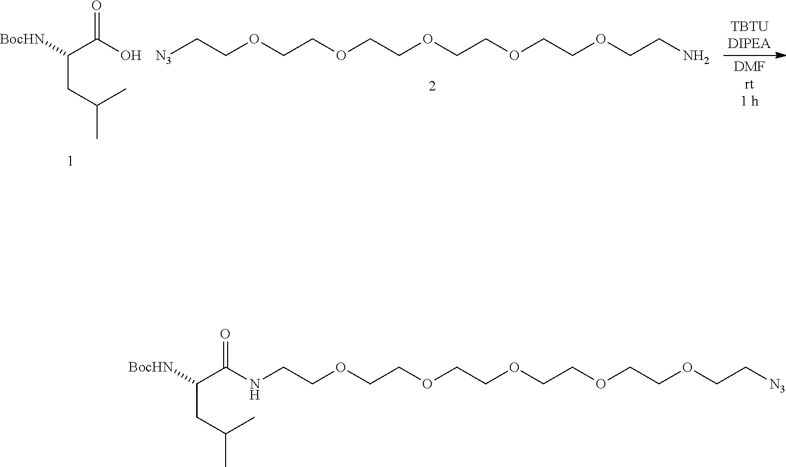

To a solution of compounds 1 (0.162 g) and 2 (0.225 g) in DMF was added TBTU (0.270 g) and then DIPEA (0.366 mL) under ambient conditions. The reaction was stirred for 1 h until full conversion was observed by LC-MS. The reaction mixture was then quenched with NaHCO₃(10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hex to EtOAc (0-100%), in which product eluted at 100% B. The product was concentrated under vacuum to provide a clear colorless oil. Yield: 0.245 g (67.4%.) LC-MS: calculated [M+H]+ 520.33 m/z, observed 520.61 m/z.

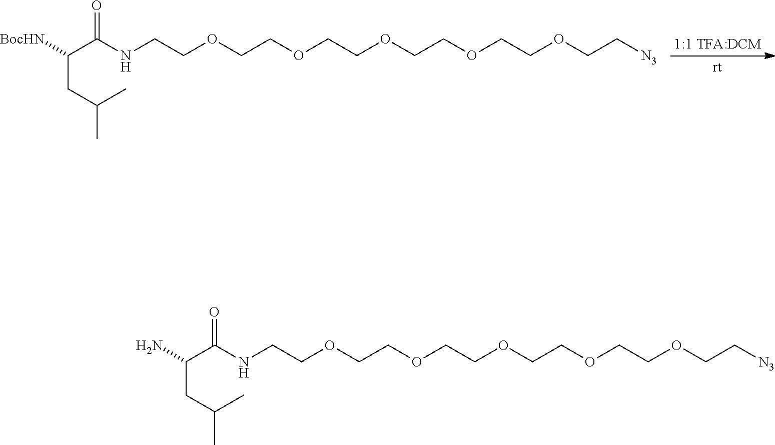 [SMILES: CC(C)C[C@H](NC(=O)OC(C)(C)C)C(=O)NCCOCCOCCOCCOCCOCCN=[N+]=[N-] ; CC(C)C[C@H](N)C(=O)NCCOCCOCCOCCOCCOCCN=[N+]=[N-]]

To a solution of compound 1 (0.245 g) in DCM was added TFA (1.08 mL) at room temperature. The reaction was stirred under ambient conditions. After 1 h, full conversion was confirmed via LC-MS. The reaction mixture was azeotroped with PhMe and subjected to a base extraction with NaHCO₃. Product was extracted with EtOAc and then 20% CF₃CH₂OH/DCM and then washed with water and brine. Mixture was then concentrated under vacuum. No isolation was necessary. Concentration provided a white solid. Yield: 0.224 (113%.) LC-MS: calculated [M+H]+ 420.27 m/z, observed 420.51 m/z.

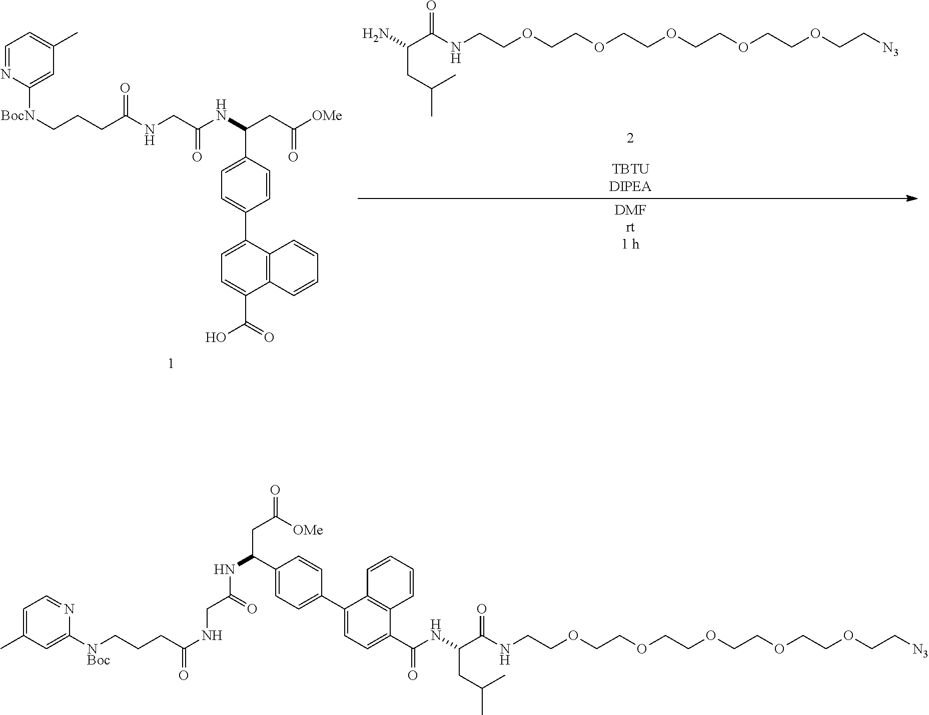

To a solution of compounds 1 (0.440 g) and 2 (0.270 g) in DMF was added TBTU (0.0248 g) and then DIPEA (0.034 mL) under ambient conditions. The reaction was stirred for 3 h until full conversion was observed by TLC. Due to scale, reaction mixture was concentrated and then resolvated in EtOAc and concentrated over silica for isolation. The residue was purified by CombiFlash® using silica gel as the stationary phase with a gradient of DCM to 20% MeOH in DCM (0-50%), in which product eluted at 18% B. The product was concentrated under vacuum to provide a clear colorless oil. Yield: 0.0475 g (68.0%.) LC-MS: calculated [M+H]+ 1084.56 m/z, observed 1085.17 m/z.

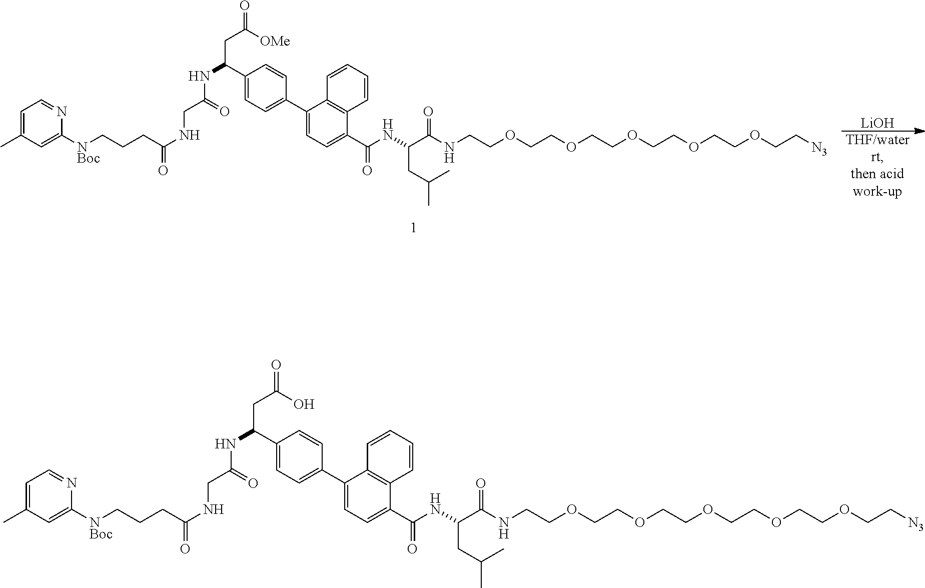

To a solution of compound 1(0.0312 g) in DCM was added TFA (0.067 mL) at room temperature. The reaction was stirred under ambient conditions overnight. The following day, full conversion was confirmed via LC-MS. The reaction mixture was then acidifed with 6 N HCl to a pH of −4, extracted with EtOAc and then 20% CF₃CH₂OH/DCM (5×8 mL), and then washed with water and brine (3×8 mL). The product was then concentrated, providing a white solid. Yield: 0.0312 g (66.5%.) LC-MS: calculated [M+H]+ 1070.55 m/z, observed 1071.12 m/z.

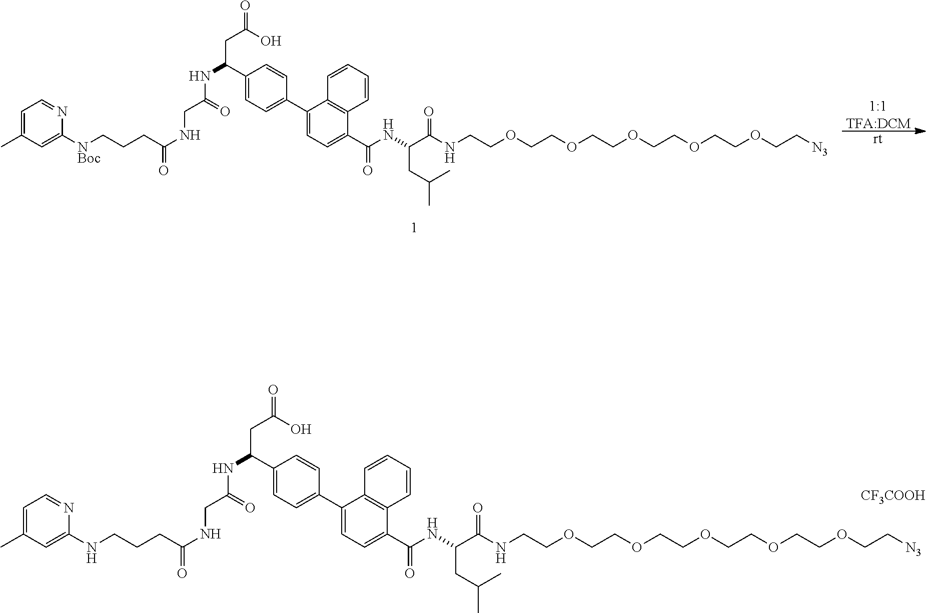

To a solution of compound 1 (0.0475 g) in 1:1 DMF/water was added LiOH (0.0031 g) at room temperature under normal atmosphere. The reaction was stirred at room temperature for 3 h until full conversion was observed by LC-MS. The reaction mixture was azeotroped with PhMe and concentrated under vacuum. No isolation was necessary. Concentration provided a clear and colorless residue. Yield: 0.0545 g (172%.) LC-MS: calculated [M+H]+ 970.50 m/z, observed 970.38 m/z.

### Synthesis of Compound 53p, (S)-3-(4-(4-(((205,235)-1-azido-20-isobutyl-19,22-dioxo-3,6,9,12,15-pentaoxa-18,21-diazapentacosan-23-yl)carbamoyl)naphthalen-1-yl)phenyl)-3-(2-(4-((4-methylpyridin-2-yl)amino)butanamido)acetamido)propanoic acid

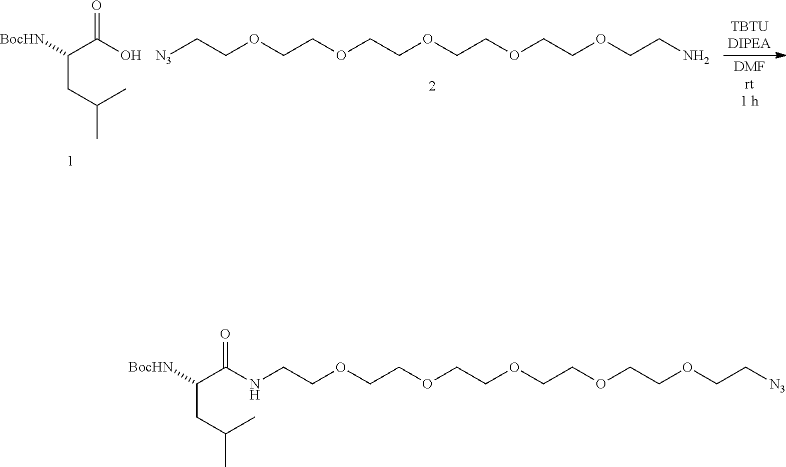

To a solution of compounds 1 (0.162 g) and 2 (0.225 g) in DMF was added TBTU (0.270 g) and then DIPEA (0.366 mL) under ambient conditions. The reaction was stirred for 1 h until full conversion was observed by LC-MS. The reaction mixture was then quenched with NaHCO₃(10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hexanes to EtOAc (0-100%), in which product eluted at 100% B. The product was concentrated under vacuum to provide a clear colorless oil. Yield: 0.245 g (67.3%.) LC-MS: calculated [M+H]+ 520.33 m/z, observed 520.61 m/z.

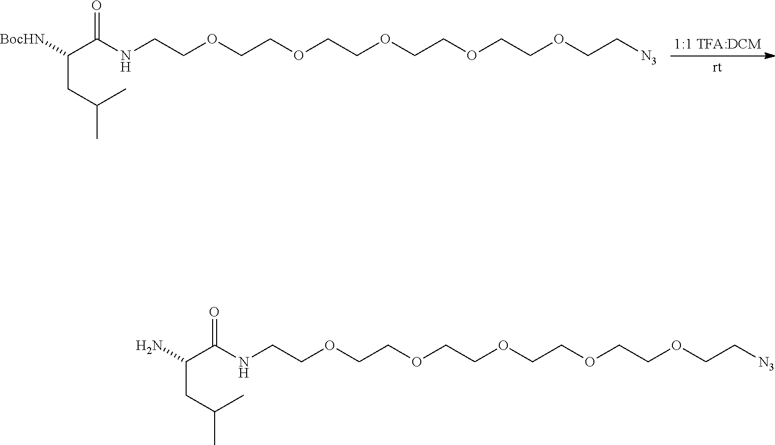 [SMILES: CC(C)C[C@H](NC(=O)OC(C)(C)C)C(=O)NCCOCCOCCOCCOCCOCCN=[N+]=[N-] ; CC(C)C[C@H](N)C(=O)NCCOCCOCCOCCOCCOCCN=[N+]=[N-]]

To a solution of compound 1 (0.245 g) in DCM was added TFA (1.61 g) at room temperature. The reaction was stirred under ambient conditions. After 1 h, full conversion was confirmed via LC-MS. The reaction mixture was azeotroped with PhMe and subjected to a base extraction with NaHCO₃. Product was extracted with EtOAc and then 20% CF₃CH₂OH/DCM and then washed with water and brine. The mixture was then concentrated under vacuum. No isolation was necessary. Concentration provided a white solid. Yield: 0.224 g (113%.) LC-MS: calculated [M+H]+ 420.27 m/z, observed 420.51 m/z.

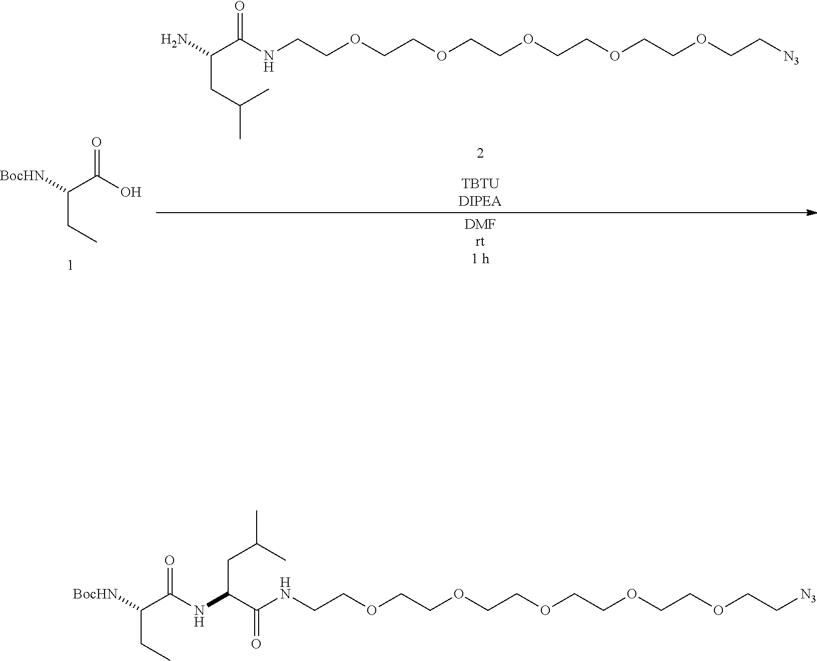

To a solution of compounds 1 (0.610 g) and 2 (0.126 g) in DMF was added TBTU (0.116 g) and then DIPEA (0.157 mL) under ambient conditions. The reaction was stirred for 1 h until full conversion was observed by LC-MS. The reaction mixture was quenched with NaHCO₃(8 mL), extracted with EtOAc and then 20% CF₃CH₂OH/DCM (3×8 mL), and then washed with water and brine (3×8 mL). Mixture was then dried over Na₂SO₄, filtered, and concentrated. The residue was purified by CombiFlash® using silica gel as the stationary phase with a gradient of DCM to 20% MeOH in DCM (0-45%), in which product eluted at 17% B. The product was concentrated under vacuum to provide a clear colorless oil. Yield: 0.110 g (60.6%) LC-MS: calculated [M+H]+ 605.38 m/z, observed 605.52 m/z.

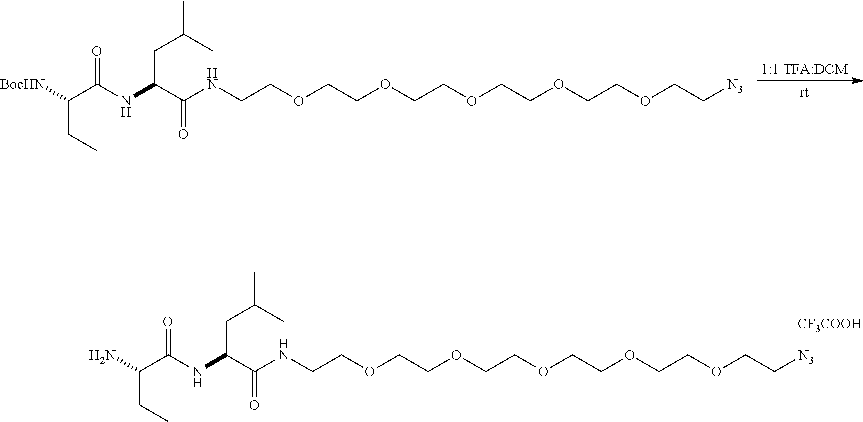

To a solution of compound 1 (0.110 g) in DCM was added TFA (0.418 mL) at room temperature. The reaction was stirred under ambient conditions. After 2 h, full conversion was confirmed via LC-MS. The reaction mixture was azeotroped with PhMe and concentrated under vacuum. No isolation was necessary. Concentration provided a clear colorless oil. Yield: 0.148 g (132%.) LC-MS: calculated [M+H]+ 505.33 m/z, observed 505.67 m/z.

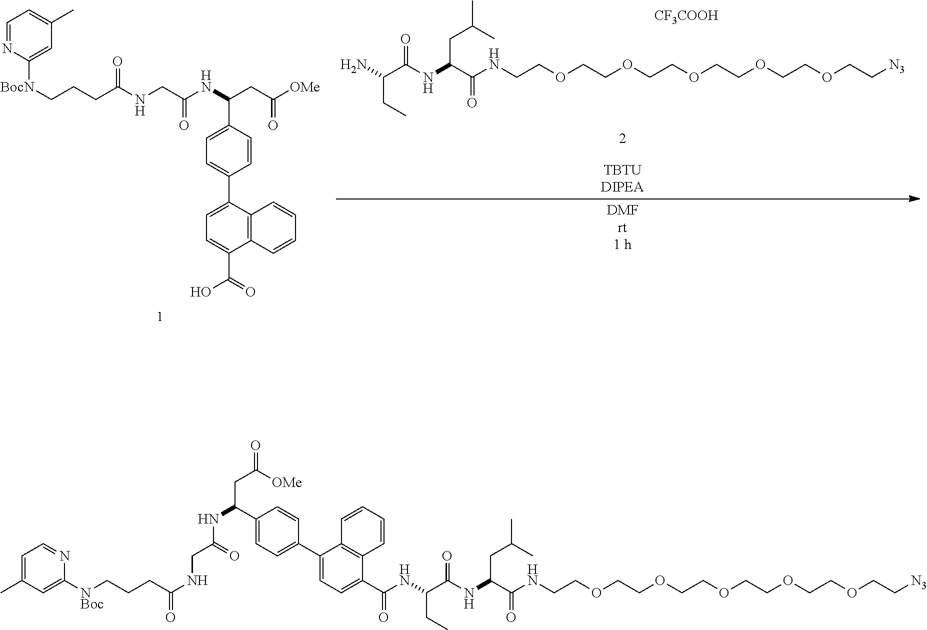

To a solution of compounds 1 (0.0440 g) and 2 (0.0399 g) in DMF was added TBTU (0.0248 g) and then DIPEA (0.034 mL) under ambient conditions. The reaction was stirred for 3 h until full conversion was observed by LC-MS. The reaction mixture was quenched with NaHCO₃(8 mL), extracted with EtOAc (3×8 mL), and then washed with water (3×8 mL). The mixture was then dried over Na₂SO₄, filtered, and concentrated. The residue was purified by CombiFlash® using silica gel as the stationary phase with a gradient of DCM to 20% MeOH in DCM (0-50%), in which product eluted at 37% B. The product was concentrated under vacuum to provide a clear colorless oil. Yield: 0.0273 g (36.2%.) LC-MS: calculated [M+H]+ 1169.62 m/z, observed 1170.59 m/z.

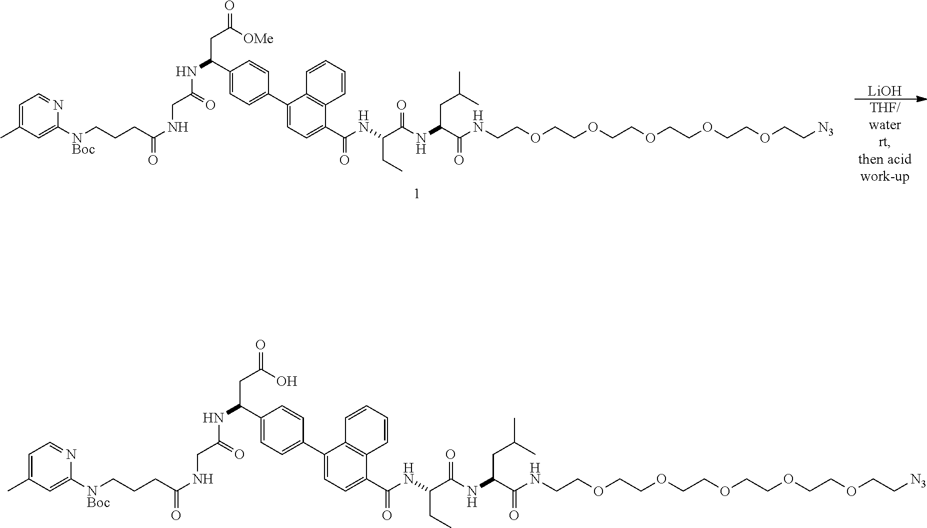

To a solution of compound 1 (0.0273 g) in 1:1 DMF/water was added LiOH (0.0017 g) at room temperature under normal atmosphere. The reaction was stirred at room temperature for 3 h until full conversion was observed by LC-MS. The reaction mixture was then acidifed with 6 N HCl to a pH of −4, extracted with EtOAc and then 20% CF₃CH₂OH/DCM (5×8 mL), and then washed with water and brine (3×8 mL). The product was then concentrated, providing a clear colorless oil. Yield: 0.0286 g (106%) LC-MS: calculated [M+H]+ 1155.60 m/z, observed 1156.30 m/z.

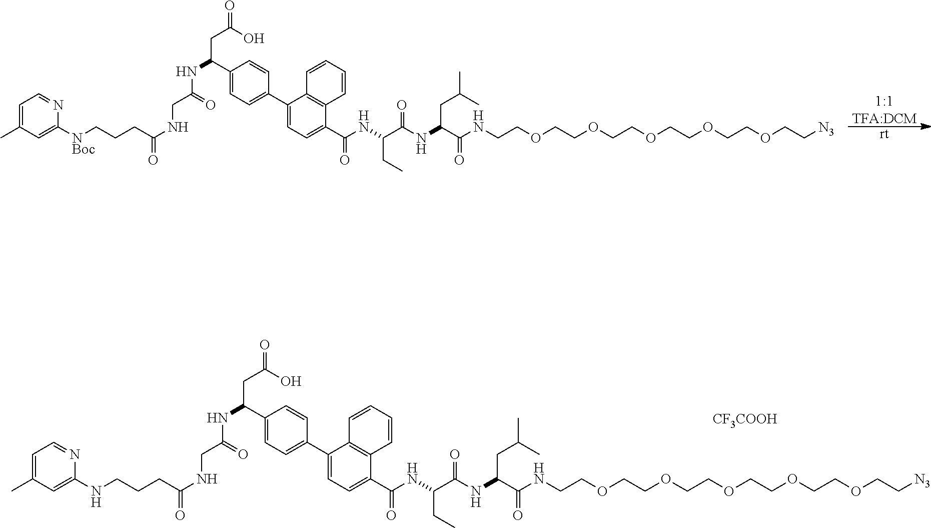

To a solution of compound 1 (0.0286 g) in DCM was added TFA (0.0847 g) at room temperature. The reaction was stirred under ambient conditions overnight. The following day, full conversion was confirmed via LC-MS. The reaction mixture was azeotroped with PhMe and concentrated under vacuum. No isolation was necessary. Concentration provided a light orange-yellow solid. Yield: 0.0384 g (133%.) LC-MS: calculated [M+H]+ 1055.55 m/z, observed 1056.08 m/z.

### Synthesis of Compound 54p, (S)-3-(4-(4-(((S)-1-azido-19-oxo-21-phenyl-3,6,9,12,15-pentaoxa-18-azahenicosan-20-yl)carbamoyl)naphthalen-1-yl)phenyl)-3-(2-(4-((4-methylpyridin-2-yl)amino)butanamido)acetamido)propanoic acid

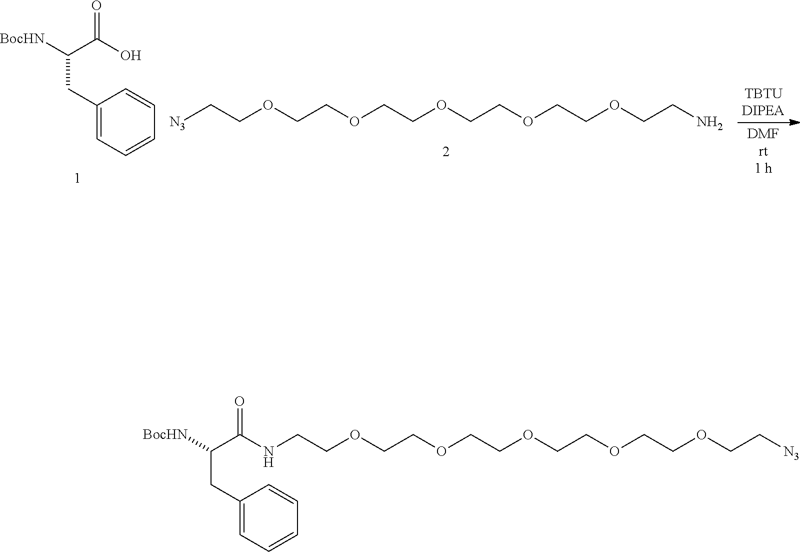

To a solution of compounds 1 (0.140 g) and 2 (0.170 g) in DMF was added TBTU (0.203 g) and then DIPEA (0.276 mL) under ambient conditions. Reaction was stirred for 1 h until full conversion was observed by LC-MS. The reaction mixture was then quenched with NaHCO₃(10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of DCM to 20% of MeOH/DCM (0-40%), in which product eluted at 14% B. The product was concentrated under vacuum to provide a clear colorless oil. Yield: 0.261 g (89.5%.) LC-MS: calculated [M+H]+ 554.31 m/z, observed 554.76 m/z.

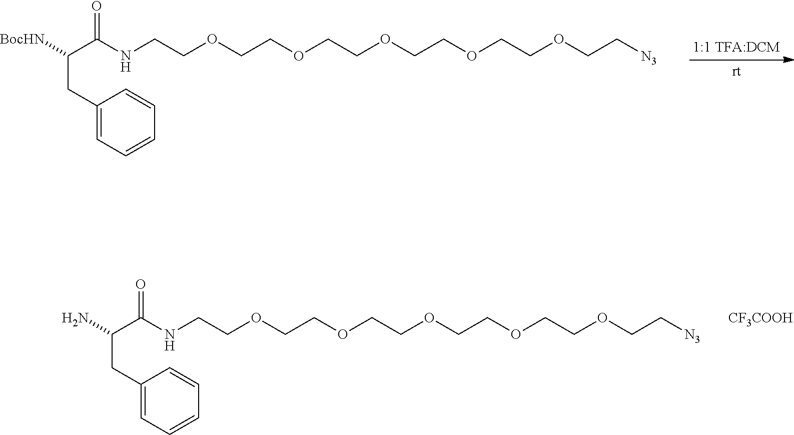

To a solution of compound 1 (0.261 g) in DCM was added TFA (1.08 mL) at room temperature. The reaction was stirred under ambient conditions. After 1 h, full conversion was confirmed via LC-MS. The reaction mixture was azeotroped with PhMe and concentrated under vacuum. No isolation was necessary. Concentration provided a yellow oil. Yield: 0.317 g (118%.) LC-MS: calculated [M+H]+ 454.26 m/z, observed 454.31 m/z.

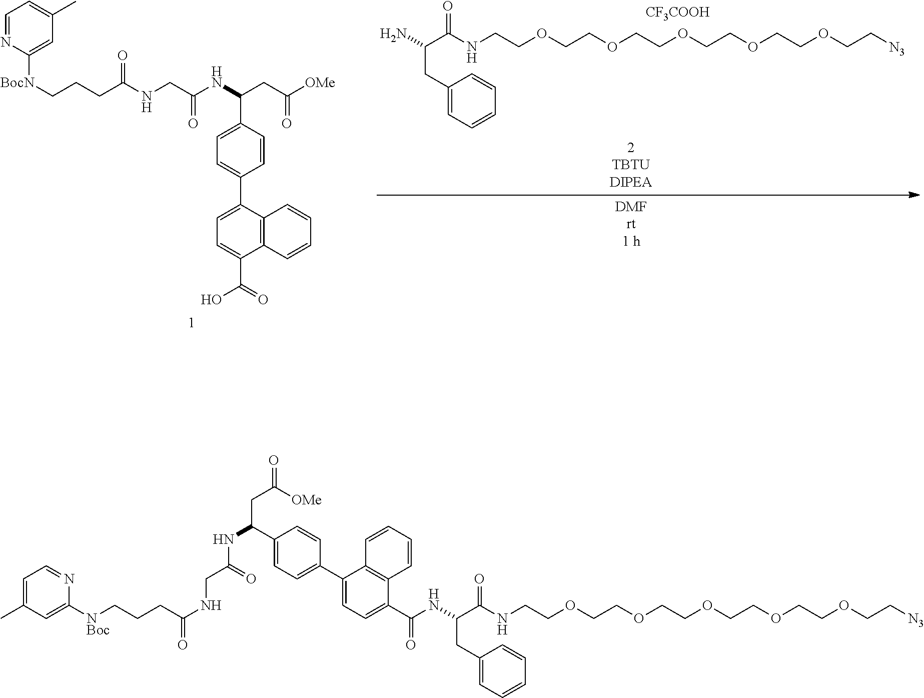

To a solution of compounds 1 (0.0400 g) and 2 (0.0333 g) in DMF was added TBTU (0.0226 g) and then DIPEA (0.031 mL) under ambient conditions. The reaction was stirred for 1 h until full conversion was observed by LC-MS. Reaction mixture was quenched with NaHCO₃(8 mL), extracted with EtOAc and then 20% CF₃CH₂OH/DCM (3×8 mL), and then washed with water (3×8 mL). The mixture was then dried over Na₂SO₄, filtered, and concentrated. The residue was purified by CombiFlash using silica gel as the stationary phase with a gradient of DCM to 20% MeOH in DCM (0-50%), in which product eluted at 30% B. The product was concentrated under vacuum to provide a clear colorless oil. Yield: 0.0386 g (58.9%.) LC-MS: calculated [M+H]+ 1118.55 m/z, observed 1119.09 m/z.

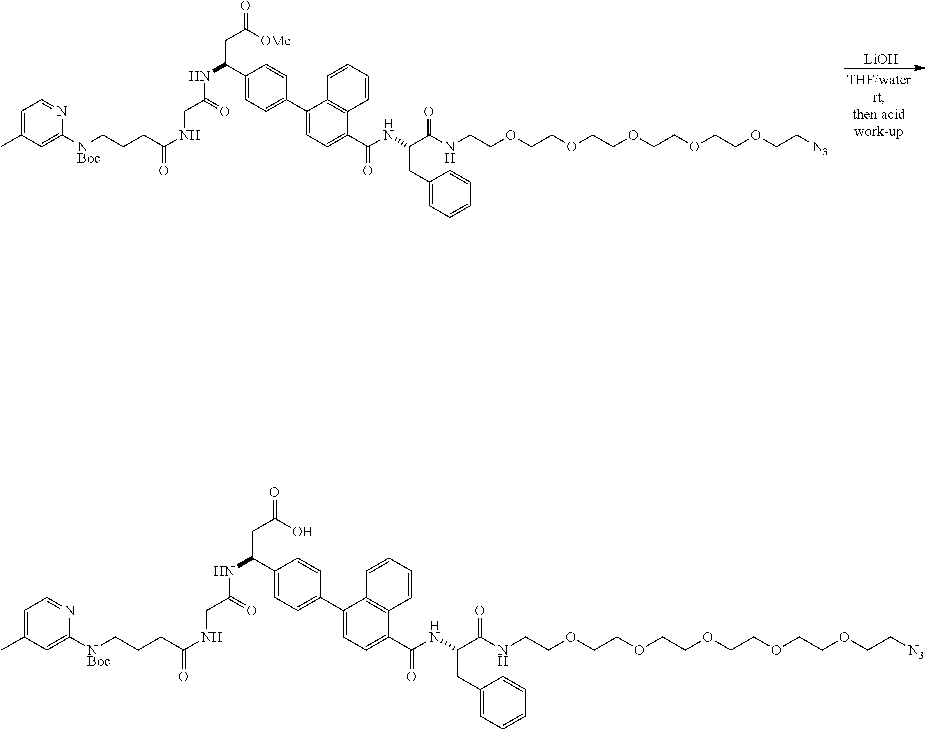

To a solution of compound 1 (0.0386 g) in 1:1 DMF/water was added LiOH (0.0025 g) at rt under normal atmosphere. The reaction was stirred at room temperature for 3 h until full conversion was observed by LC-MS. The reaction mixture was then acidifed with 6 N HCl to a pH of −4, extracted with EtOAc and then 20% CF₃CH₂OH/DCM (5×8 mL), and then washed with water and brine (3×8 mL). The product was then concentrated, providing a white solid. Yield: 0.0665 g (174%.) LC-MS: calculated [M+H]+ 1104.53 m/z, observed 1105.05 m/z.

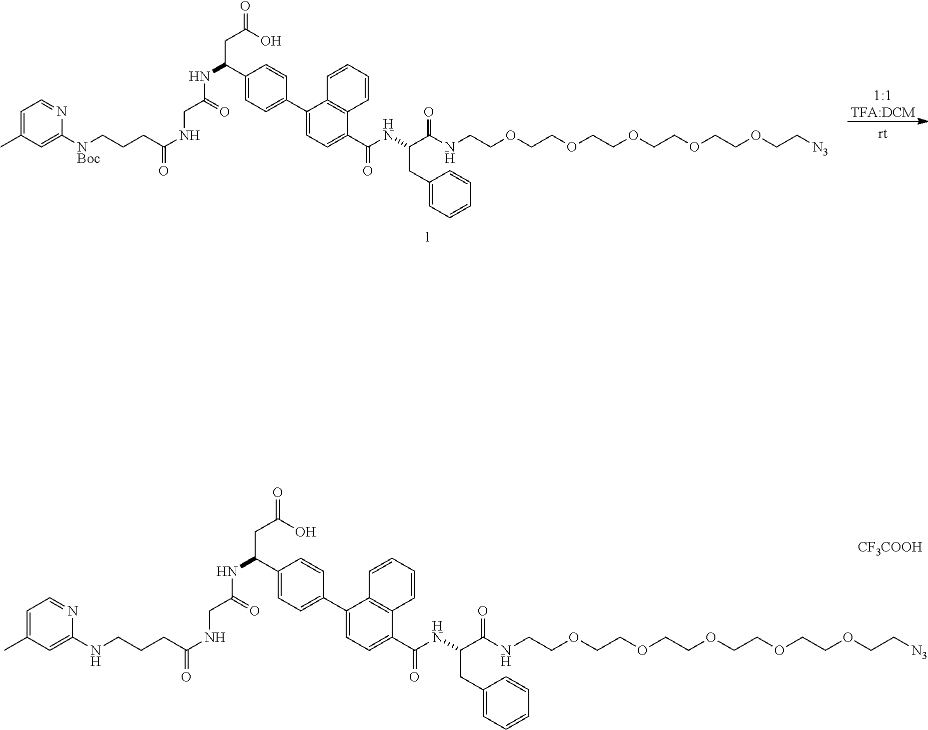

To a solution of compound 1 (0.0665 g) in DCM was added TFA (0.138 mL) at room temperature. The reaction was stirred under ambient conditions for 3 h until full conversion was confirmed via LC-MS. The reaction mixture was azeotroped with PhMe and concentrated under vacuum. No isolation was necessary. Concentration provided an off-white solid. Yield: 0.0911 g (135%.) LC-MS: calculated [M+H]+ 1004.48 m/z, observed 1005.55 m/z. Synthesis of Compound 55p, (S)-3-(4-(4-((14-azido-3,6,9,12-tetraoxatetradecyl)oxy)naphthalen-1-yl)phenyl)-3-(2-(5-((4-methylpyrimidin-2-yl)amino)pentanamido)acetamido)propanoic acid

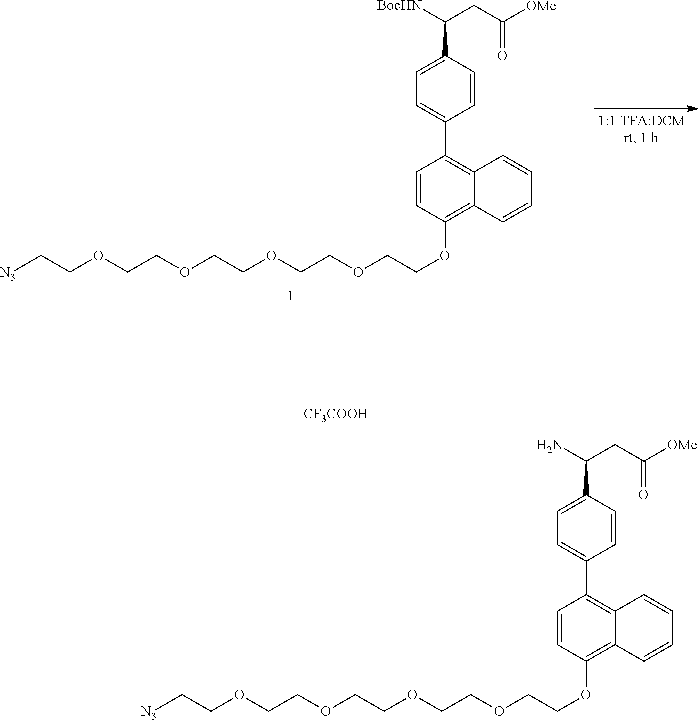 [SMILES: COC(=O)C[C@H](NC(=O)OC(C)(C)C)c1ccc(-c2ccc(OCCOCCOCCOCCOCCN=[N+]=[N-])c3ccccc23)cc1 ; COC(=O)C[C@H](N)c1ccc(-c2ccc(OCCOCCOCCOCCOCCN=[N+]=[N-])c3ccccc23)cc1 ; "CF3COOH"]

To a solution of compound 1 (0.126 g) in DCM was added TFA (0.433 mL) at room temperature. The reaction was stirred under ambient conditions. After 2 h, full conversion was confirmed via LC-MS. The reaction mixture was azeotroped with PhMe and concentrated under vacuum. No isolation was necessary. Concentration provided a yellow oil. Yield: 0.134 g (104%) LC-MS: calculated [M+H]+ 567.27 m/z, observed 567.58m/z.

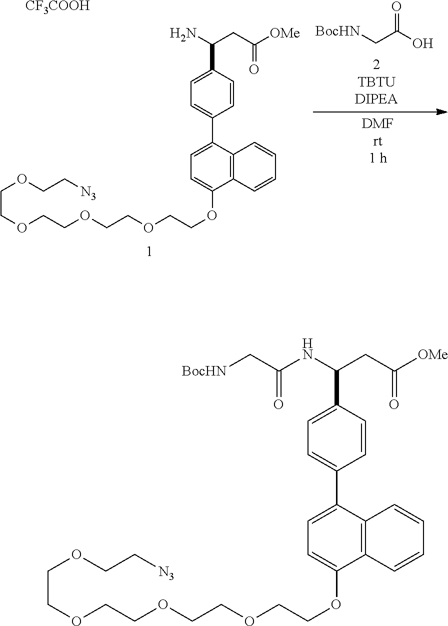

To a solution of compounds 1 (0.134 g) and 2 (0.0344 g) in DMF was added TBTU (0.0757 g) and then DIPEA (0.103 mL) under ambient conditions. The reaction was stirred for 3 h until full conversion was observed by TLC. The reaction mixture was then quenched with NaHCO₃(8 mL). The product was extracted with EtOAc (3×8 mL) and then 20% CF₃CH₂OH/DCM (1×8 mL) and then washed with brine (1×8 mL) and then water (3×8 mL). The combined organic phase was dried over Na₂SO₄, filtered, and concentrated. The residue was purified by CombiFlash® using silica gel as the stationary phase with a gradient of DCM to 20% MeOH in DCM (0-40%), in which product eluted at 14% B. The product was concentrated under vacuum to provide a clear colorless residue. Yield: 0.0999 g (70.3%.) LC-MS: calculated [M+H]+ 724.35 m/z, observed 724.92 m/z.

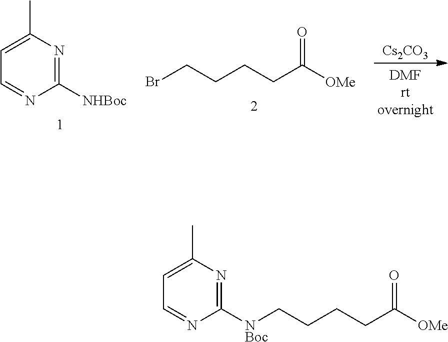

To a solution of compound 1 (0.100 g) in DMF was added Cs₂CO₃ (0.234 g) under ambient conditions. Compound 2 (0.068 mL) was then added slowly. The reaction was stirred overnight. Approx. 50% conversion to desired product by LC-MS was then confirmed. The reaction mixture was quenched with NaHCO₃(10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of hex to EtOAc (0-70%), in which product eluted at 29% B. The product was concentrated under vacuum to provide a clear colorless oil. Yield: 0.0993 g (64.3%.) LC-MS: calculated [M+H]+ 324.18 m/z, observed 324.41 m/z.

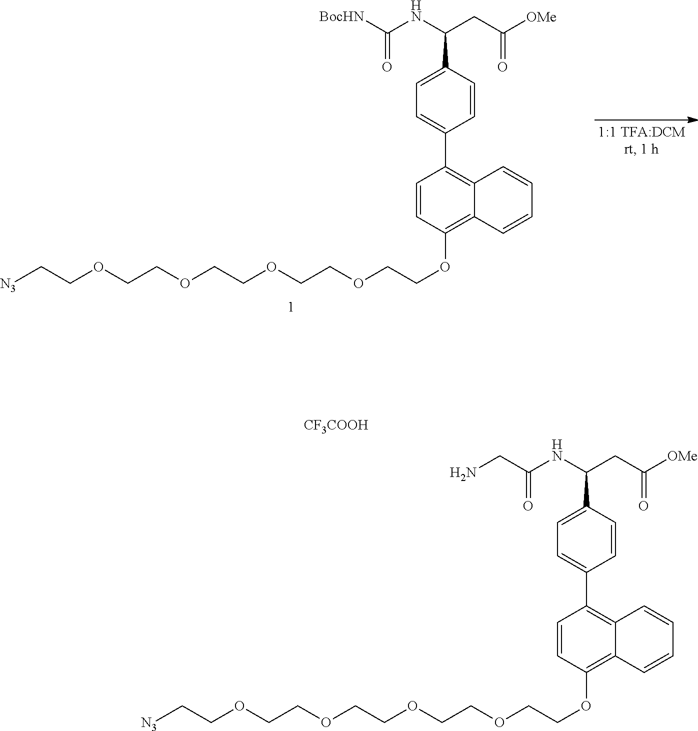 [SMILES: COC(=O)C[C@H](NC(=O)NC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.0999 g) in DCM was added TFA (0.317 mL) at room temperature. The reaction was stirred under ambient conditions. After 5 h, full conversion was confirmed via TLC. The reaction mixture was azeotroped with PhMe and concentrated under vacuum. No isolation was necessary. Concentration provided a yellow oil. Yield: 0.1168 g (115%.) LC-MS: calculated [M+H]+ 624.30 m/z, observed 624.68 m/z.

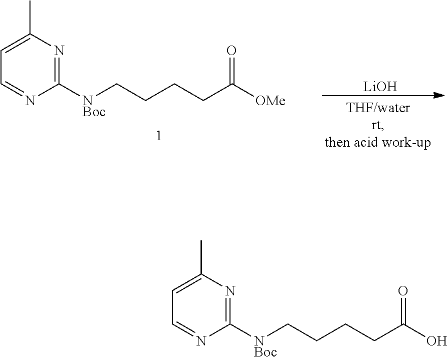 [SMILES: COC(=O)CCCCN(C(=O)OC(C)(C)C)c1nccc(C)n1 ; "LiOH" ; Cc1ccnc(N(CCCCC(=O)O)C(=O)OC(C)(C)C)n1]

To a solution of compound 1 (0.0993 g) in 1:1 THF/water was added LiOH (0.0221 g) at room temperature under normal atmosphere. The reaction was stirred at room temperature until full conversion was observed by TLC. After 4 h, the reaction mixture was acidifed with 6 N HCl to a pH of −3. The product was extracted with EtOAc (3×5 mL) and then 20% CF₃CH₂OH/DCM (3×5 mL). The combined organic phase was dried over Na₂SO₄, filtered, and concentrated, providing a clear, colorless oil. Yield: 0.0876 g (92.2%.) LC-MS: calculated [M+H]+ 310.17 m/z, observed 310.49 m/z.

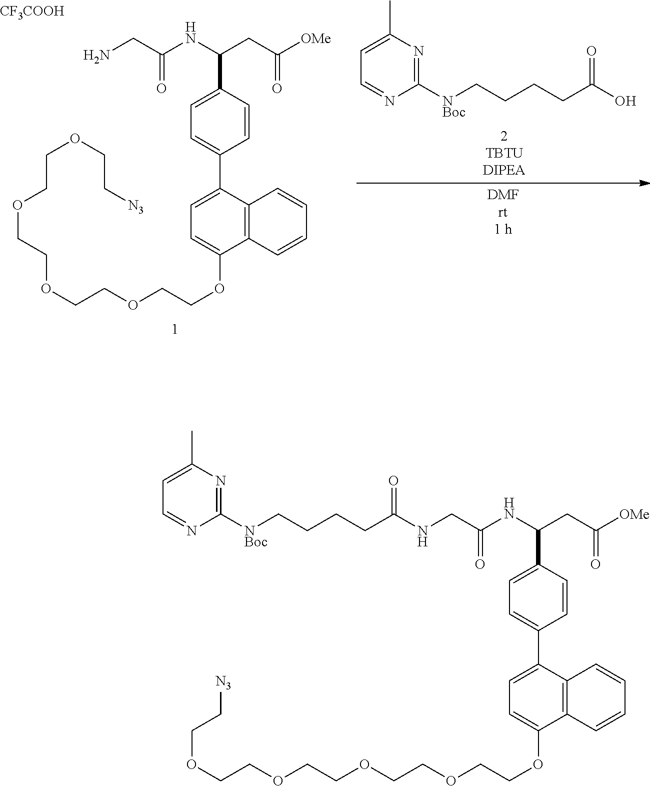

To a solution of compounds 1 (0.113 g) and 2 (0.0472 g) in DMF was added DIPEA (0.080 mL) and then TBTU (0.0588 g) under ambient conditions. Reaction was stirred for 1 h until full conversion was observed by TLC. The reaction mixture was then quenched with NaHCO₃(10 mL). The product was extracted with EtOAc (3×5 mL) and then 20% CF₃CH₂OH/DCM (3×8 mL) and then washed with water (3×10 mL). The combined organic phase was dried over Na₂SO₄, filtered, and concentrated. The residue was purified by CombiFlash using silica gel as the stationary phase with a gradient of DCM to 20% MeOH in DCM (0-70%), in which product eluted at 34% B. The product was concentrated under vacuum to provide a clear colorless oil. Yield: 0.0712 g (51.0%.) LC-MS: calculated [M+H]+ 915.45 m/z, observed 915.85 m/z.

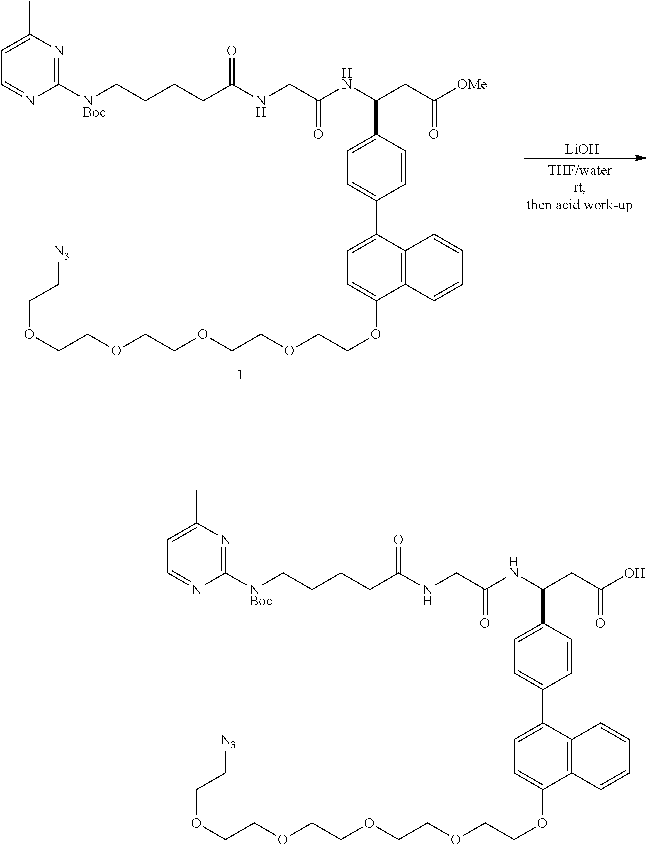

To a solution of compound 1 (0.0712 g) in 1:1 THF/water was added LiOH (0.0056 g) at rt under normal atmosphere. The reaction was stirred at room temperature until full conversion was observed by LC-MS. After 4 h, the reaction mixture was acidifed with 6 N HCl to a pH of −3. The product was extracted with EtOAc and then 20% CF₃CH₂OH/DCM (3×8 mL). The combined organic phase was dried over Na₂SO₄, filtered, and concentrated, providing a clear colorless oil. LC-MS: calculated [M+H]+ 901.44 m/z, observed 901.57 m/z.

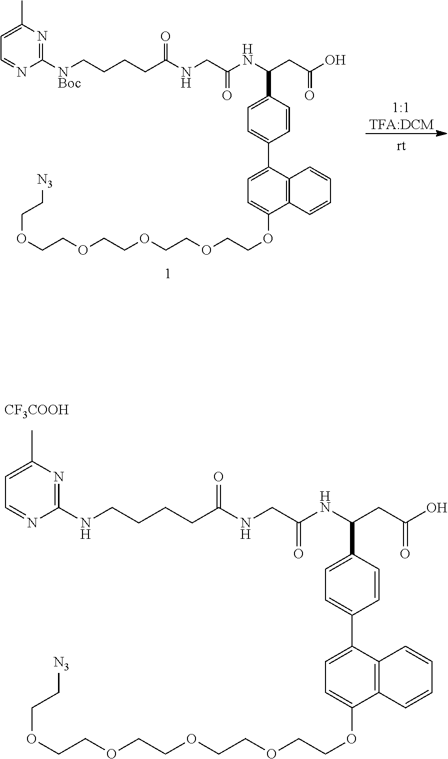

To a solution of compound 1 (0.0640 g) in DCM was added TFA (0.163 mL) at room temperature. The reaction was stirred under ambient conditions overnight. The following day, desired product was observed via LC-MS. The reaction mixture was azeotroped with PhMe and concentrated under vacuum to provide a clear colorless oil. Yield: 0.0707 g (108%.) LC-MS: calculated [M+H]+ 801.39 m/z, observed 801.47 m/z.

### Synthesis of Compound 56p, (S)-3-(4-(4-((14-azido-3,6,9,12-tetraoxatetradecyl)oxy)naphthalen-1-yl)phenyl)-3-(2-(5-((6-methylpyridin-2-yl)amino)pentanamido)acetamido)propanoic acid

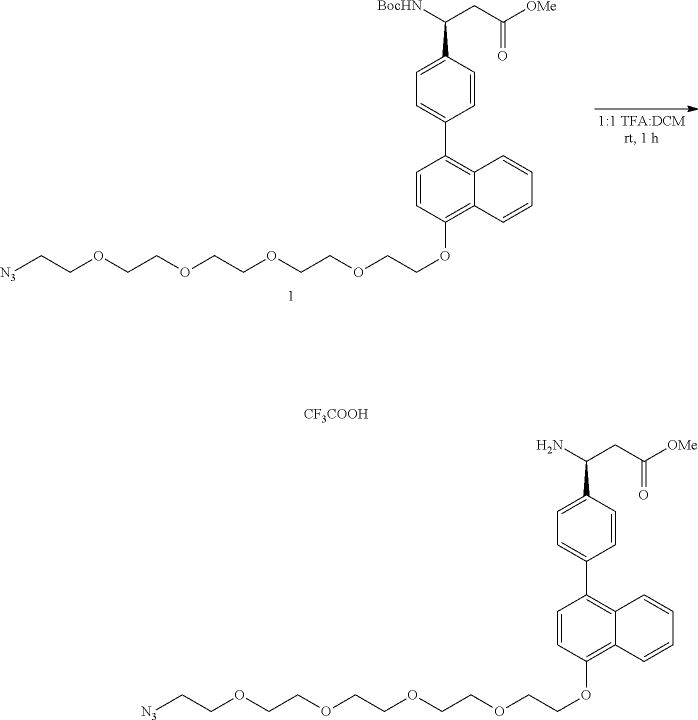 [SMILES: COC(=O)C[C@H](NC(=O)OC(C)(C)C)c1ccc(-c2ccc(OCCOCCOCCOCCOCCN=[N+]=[N-])c3ccccc23)cc1 ; COC(=O)C[C@H](N)c1ccc(-c2ccc(OCCOCCOCCOCCOCCN=[N+]=[N-])c3ccccc23)cc1 ; "CF3COOH"]

To a solution of compound 1 (0.356 g) in DCM was added TFA (1.227 mL) at room temperature. The reaction was stirred under ambient conditions. After 2 h, full conversion was confirmed via LC-MS. The reaction mixture was azeotroped with PhMe and concentrated under vacuum. No isolation was necessary. Concentration provided a deep honey-colored oil. Yield: 0.364 g (100%.) LC-MS: calculated [M+H]+ 567.27 m/z, observed 567.58 m/z.

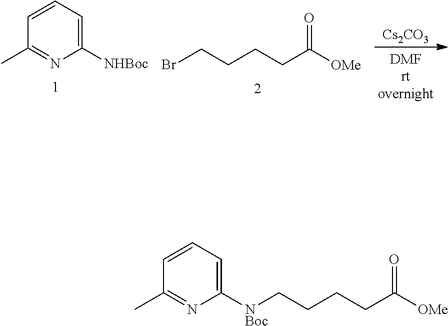

To a solution of compound 1 (0.0961 g) in DMF was added Cs₂CO₃ (0.226 g) under ambient conditions. Compound 2 (0.066 mL) was then added slowly. The reaction was stirred overnight. Approx. 50% conversion to desired product by LC-MS was then confirmed. The reaction mixture was quenched with NaHCO₃(10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hex to EtOAc (0-30%), in which product eluted at 19% B. The product was concentrated under vacuum to provide a clear colorless oil. Yield: 0.0354 g (23.8%.) LC-MS: calculated [M+H]+ 323.19 m/z, observed 323.10 m/z.

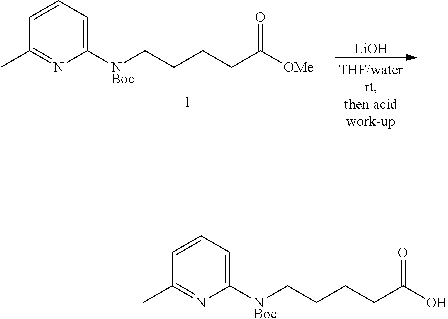 [SMILES: COC(=O)CCCCN(C(=O)OC(C)(C)C)c1cccc(C)n1 ; "LiOH" ; Cc1cccc(N(CCCCC(=O)O)C(=O)OC(C)(C)C)n1]

To a solution of compound 1 (0.0354 g) in 1:1 THE/water was added LiOH (0.0079 g) at room temperature under normal atmosphere. The reaction was stirred at room temperature until full conversion was observed by TLC. After 1 h, the reaction mixture was acidifed with 6 N HCl to a pH of −3. The product was extracted with EtOAc and then 20% CF₃CH₂OH/DCM (3×8 mL). The combined organic phase was dried over Na₂SO₄, filtered, and concentrated, providing a clear, colorless oil. Yield: 0.0625 g (184%.) LC-MS: calculated [M+H]+ 309.17 m/z, observed 309.42 m/z.

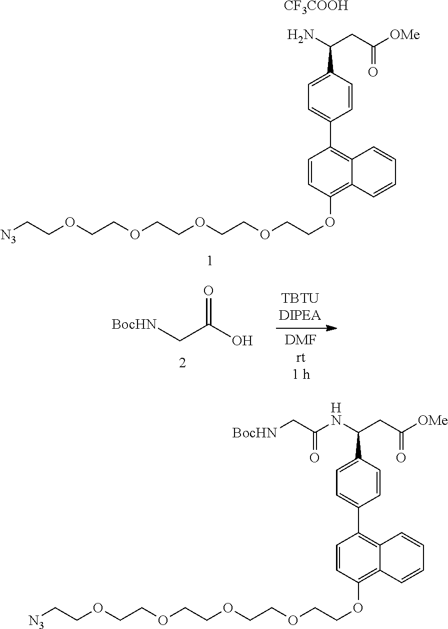 [SMILES: COC(=O)C[C@H](N)c1ccc(-c2ccc(OCCOCCOCCOCCOCCN=[N+]=[N-])c3ccccc23)cc1 ; "CF3COOH" ; CC(C)(C)OC(=O)NCC(=O)O ; COC(=O)C[C@H](NC(=O)CNC(=O)OC(C)(C)C)c1ccc(-c2ccc(OCCOCCOCCOCCOCCN=[N+]=[N-])c3ccccc23)cc1]

To a solution of compounds 1 (0.364 g) and 2 (0.0936 g) in DMF was added TBTU (0.206 g) and then DIPEA (0.279 mL) under ambient conditions. Reaction was stirred for 3 h. Then reaction mixture was quenched with NaHCO₃(10 mL) and brine (15 mL). The product was extracted with EtOAc (2×5 mL) and then 20% CF₃CH₂OH/DCM (3×8 mL) and then washed with water (5×8 mL) and brine (1×5 mL). The combined organic phase was dried over Na₂SO₄, filtered, and concentrated. The residue was purified by CombiFlash® using silica gel as the stationary phase with a gradient of DCM to 20% MeOH in DCM (0-25%), in which product eluted at 5% B to provide a clear colorless oil. Yield: 0.243 g (63.0%.) LC-MS: calculated [M+H]+ 724.35 m/z, observed 724.66 m/z.

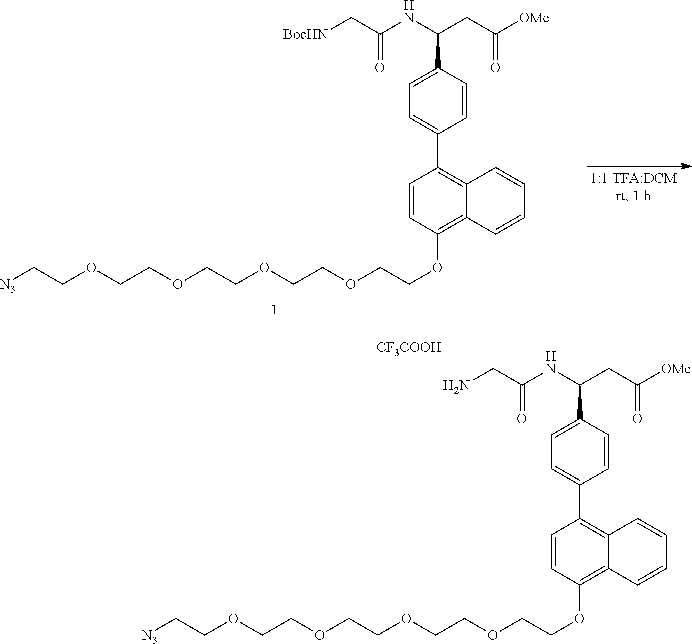 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.244 g) in DCM was added TFA (0.774 mL) at room temperature. The reaction was stirred under ambient conditions. After 1 h, full conversion was confirmed via LC-MS. The reaction mixture was concentrated under vacuum. No isolation was necessary. Concentration provided a yellow oil. Yield: 0.281 g (113%.) LC-MS: calculated [M+H]+ 624.30 m/z, observed 624.56m/z.

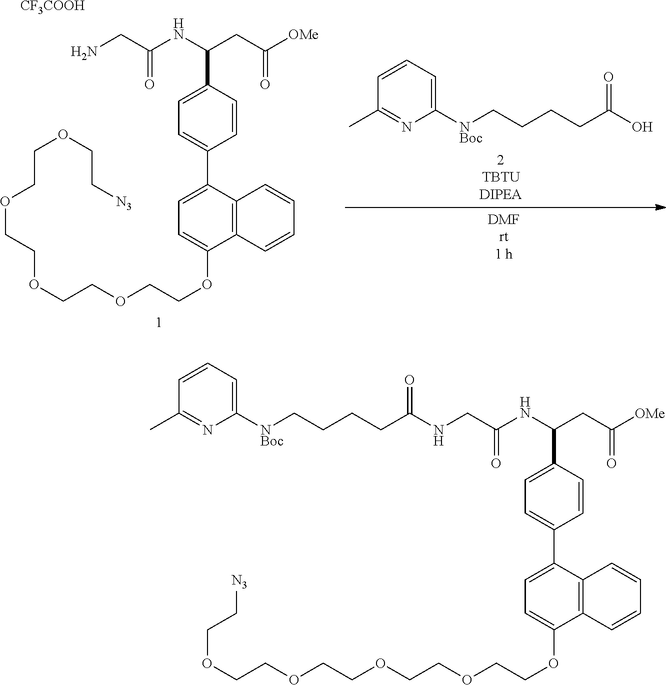 [SMILES: "CF3COOH" ; COC(=O)C[C@H](NC(=O)CN)c1ccc(-c2ccc(OCCOCCOCCOCCOCCN=[N+]=[N-])c3ccccc23)cc1 ; Cc1cccc(N(CCCCC(=O)O)C(=O)OC(C)(C)C)n1 ; COC(=O)C[C@H](NC(=O)CNC(=O)CCCCN(C(=O)OC(C)(C)C)c1cccc(C)n1)c1ccc(-c2ccc(OCCOCCOCCOCCOCCN=[N+]=[N-])c3ccccc23)cc1]

To a solution of compounds 1 (0.115 g) and 2 (0.0625 g) in DMF was added TBTU (0.0601 g) and then DIPEA (0.081 mL) under ambient conditions. The reaction was stirred for 3 h until full conversion was observed by LC-MS. The reaction mixture was then quenched with NaHCO₃(8 mL). The product was extracted with EtOAc (2×5 mL) and then 20% CF₃CH₂OH/DCM (3×8 mL) and then washed with water (3×8 mL) and brine (8 mL). The combined organic phase was dried over Na₂SO₄, filtered, and concentrated. The residue was purified by CombiFlash® using silica gel as the stationary phase with a gradient of DCM to 20% MeOH in DCM (0-30%), in which product eluted at 20% B. The product was concentrated under vacuum to provide a clear colorless oil. Yield: 0.0450 g (31.6%.) LC-MS: calculated [M+H]+ 914.46 m/z, observed 914.79 m/z.

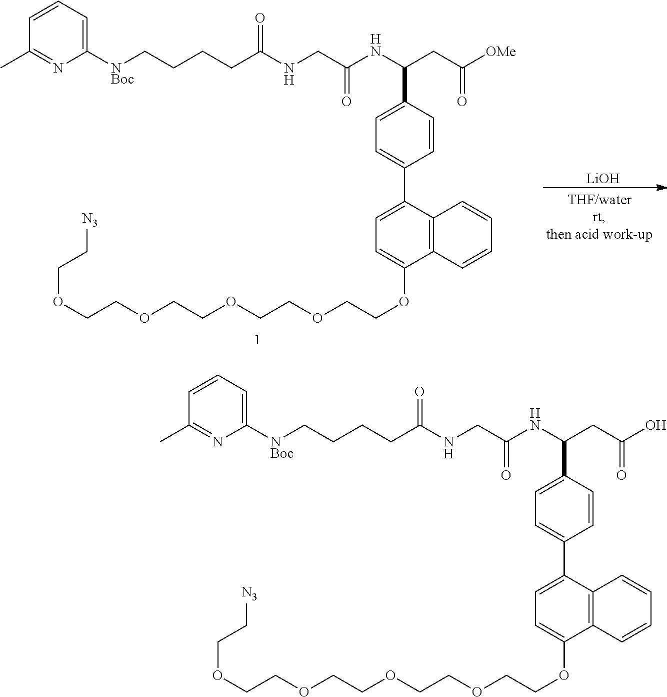 [SMILES: COC(=O)C[C@H](NC(=O)CNC(=O)CCCCN(C(=O)OC(C)(C)C)c1cccc(C)n1)c1ccc(-c2ccc(OCCOCCOCCOCCOCCN=[N+]=[N-])c3ccccc23)cc1 ; "LiOH" ; Cc1cccc(N(CCCCC(=O)NCC(=O)N[C@@H](CC(=O)O)c2ccc(-c3ccc(OCCOCCOCCOCCOCCN=[N+]=[N-])c4ccccc34)cc2)C(=O)OC(C)(C)C)n1]

To a solution of compound 1 (0.450 g) in 1:1 THF/water was added LiOH (0.0035 g) at rt under normal atmosphere. The reaction was stirred at room temperature until full conversion was observed by LC-MS. After 2 h, the reaction mixture was acidifed with 6 N HCl to a pH of −3. The product was extracted with EtOAc and then 20% CF₃CH₂OH/DCM (3×8 mL). The combined organic phase was dried over Na₂SO₄, filtered, and concentrated, providing a clear colorless oil. Yield: 0.0425 g (95.9%.) LC-MS: calculated [M+H]+ 900.44 m/z, observed 900.74 m/z.

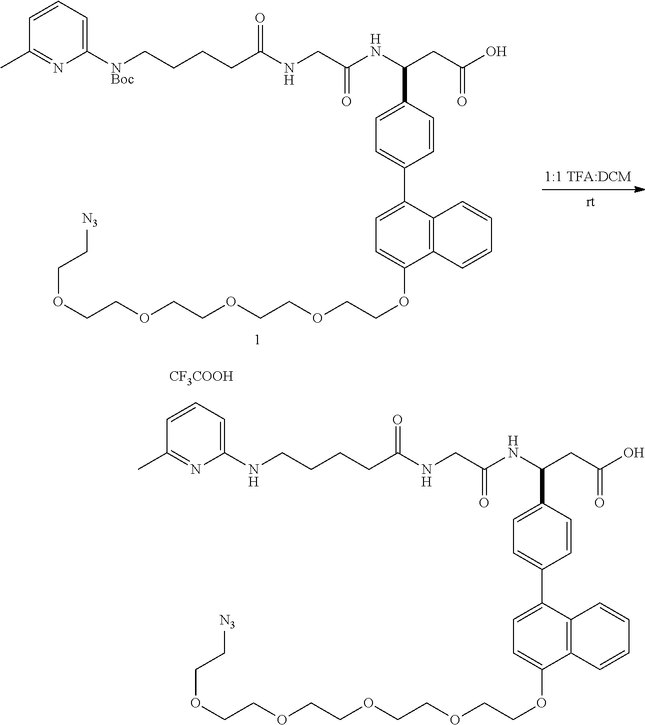 [SMILES: Cc1cccc(N(CCCCC(=O)NCC(=O)N[C@@H](CC(=O)O)c2ccc(-c3ccc(OCCOCCOCCOCCOCCN=[N+]=[N-])c4ccccc34)cc2)C(=O)OC(C)(C)C)n1 ; "CF3COOH" ; Cc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1]

To a solution of compound 1 (0.0425 g) in DCM was added TFA (0.108 mL) at room temperature. The reaction was stirred overnight under ambient conditions until full conversion was observed by LC-MS. The reaction mixture was azeotroped with PhMe and concentrated under vacuum to provide a light yellow oil. Yield: 0.0468 g (108%) LC-MS: calculated [M+H]+ 800.39 m/z, observed 800.73 m/z.

### Synthesis of Compound 57p, (S)-3-(4-(4-((14-azido-3,6,9,12-tetraoxatetradecyl)oxy)naphthalen-1-yl)phenyl)-3-(2-(5-((6-methoxypyridin-2-yl)amino)pentanamido)acetamido)propanoic acid

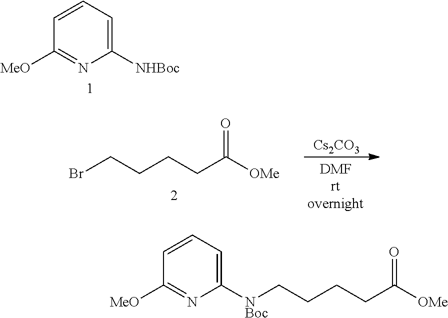

To a solution of compound 1 (0.1035 g) in DMF was added Cs₂CO₃ (0.226 g) under ambient conditions. Compound 2 (0.066 mL) was then added slowly. Reaction was stirred overnight. Approx. 50% conversion to desired product by LC-MS was then confirmed. The reaction mixture was quenched with NaHCO₃(10 mL). The product was extracted with EtOAc (3×15 mL) and then washed with water (3×10 mL). The combined organic phase was dried over Na₂SO₄, filtered, and concentrated. The residue was purified by CombiFlash® using silica gel as the stationary phase with a gradient of hex to EtOAc (0-15%), in which product eluted at 6% B. The product was concentrated under vacuum to provide a clear colorless oil. Yield: 0.0438 g (28.0%.) LC-MS: calculated [M+H]+ 339.18 m/z, observed 339.48 m/z.

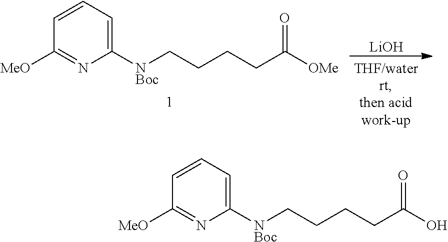 [SMILES: COC(=O)CCCCN(C(=O)OC(C)(C)C)c1cccc(OC)n1 ; "LiOH" ; COc1cccc(N(CCCCC(=O)O)C(=O)OC(C)(C)C)n1]

To a solution of compound 1 (0.0438 g) in 1:1 THF/water was added LiOH (0.0093 g) at room temperature under normal atmosphere. The reaction was stirred at room temperature until full conversion was observed by TLC. After 1 h, the reaction mixture was acidifed with 6 N HCl to a pH of −3. The product was extracted with EtOAc and then 20% CF₃CH₂OH/DCM (3×8 mL). The combined organic phase was dried over Na₂SO₄, filtered, and concentrated, providing a clear, colorless oil. Yield: 0.0485 g (115%.) LC-MS: calculated [M+H]+ 325.17 m/z, observed 325.35 m/z.

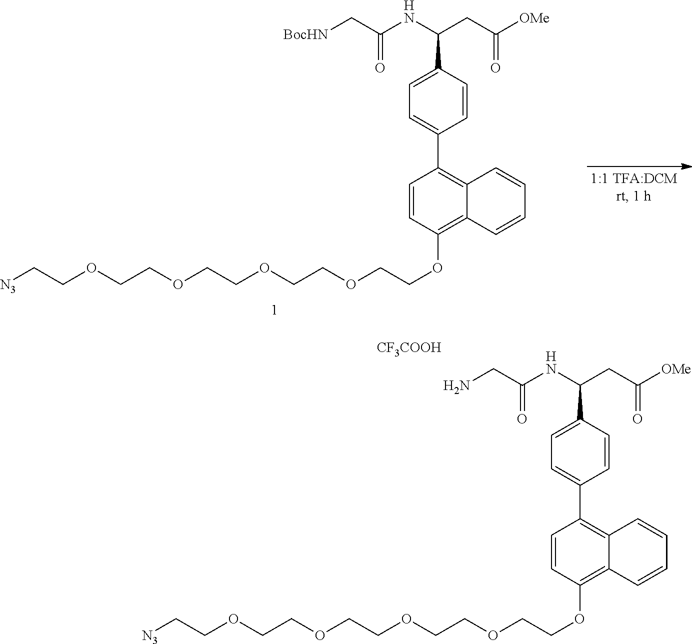 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.244 g) in DCM was added TFA (0.774 mL) at room temperature. The reaction was stirred under ambient conditions. After 1 h, full conversion was confirmed via LC-MS. The reaction mixture was concentrated under vacuum. No isolation was necessary. Concentration provided a yellow oil. Yield: 0.281 g (113%.) LC-MS: calculated [M+H]+ 624.30 m/z, observed 624.56 m/z.

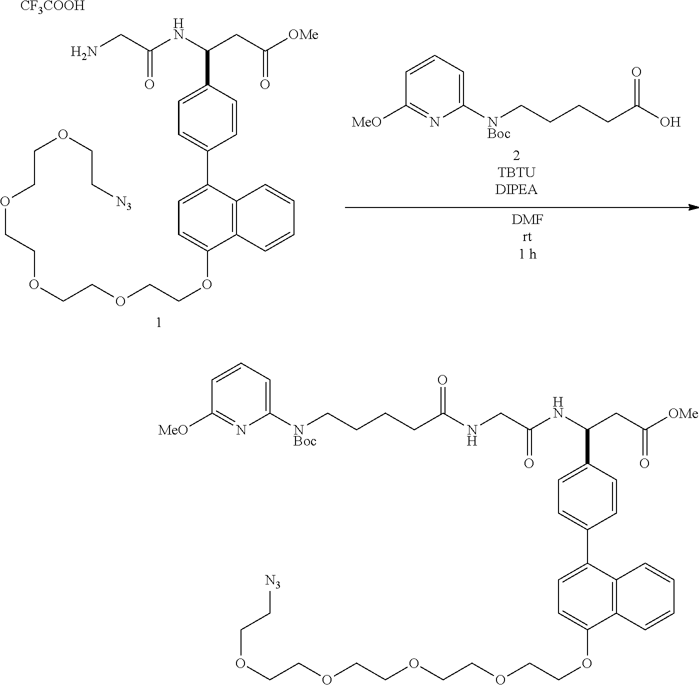 [SMILES: "CF3COOH" ; COC(=O)C[C@H](NC(=O)CN)c1ccc(-c2ccc(OCCOCCOCCOCCOCCN=[N+]=[N-])c3ccccc23)cc1 ; COc1cccc(N(CCCCC(=O)O)C(=O)OC(C)(C)C)n1 ; COC(=O)C[C@H](NC(=O)CNC(=O)CCCCN(C(=O)OC(C)(C)C)c1cccc(OC)n1)c1ccc(-c2ccc(OCCOCCOCCOCCOCCN=[N+]=[N-])c3ccccc23)cc1]

To a solution of compounds 1 (0.0850 g) and 2 (0.0486 g) in DMF was added TBTU (0.0444 g) and then DIPEA (0.060 mL) under ambient conditions. Reaction was stirred for 3 h until full conversion was observed by LC-MS. The reaction mixture was then quenched with NaHCO₃(8 mL). The product was extracted with EtOAc (2×5 mL) and then 20% CF₃CH₂OH/DCM (3×8 mL) and then washed with water (3×8 mL) and brine (8 mL). The combined organic phase was dried over Na₂SO₄, filtered, and concentrated. The residue was purified by CombiFlash® using silica gel as the stationary phase with a gradient of DCM to 20% MeOH in DCM (0-30%), in which product eluted at 17% B. The product was concentrated under vacuum to provide a clear colorless oil. Yield: 0.0518 g (48.3%.) LC-MS: calculated [M+H]+ 930.45 m/z, observed 930.90 m/z.

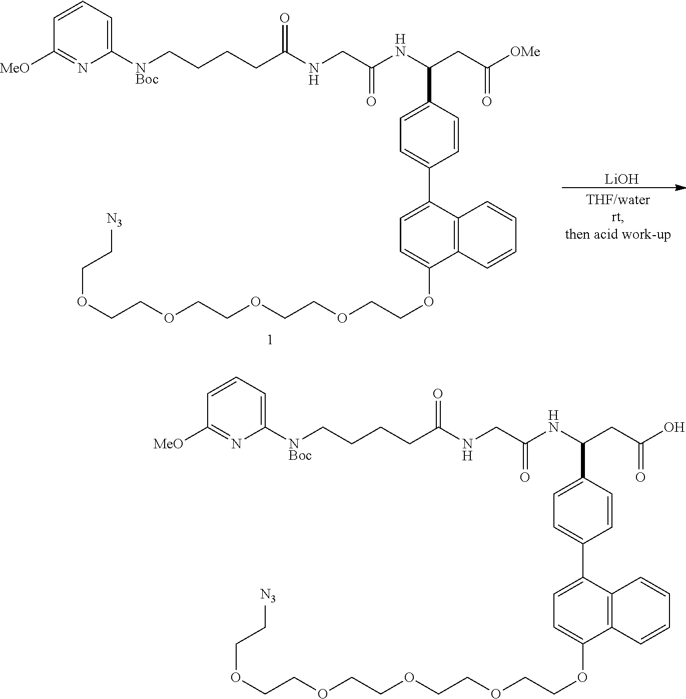 [SMILES: COC(=O)C[C@H](NC(=O)CNC(=O)CCCCN(C(=O)OC(C)(C)C)c1cccc(OC)n1)c1ccc(-c2ccc(OCCOCCOCCOCCOCCN=[N+]=[N-])c3ccccc23)cc1 ; "LiOH" ; COc1cccc(N(CCCCC(=O)NCC(=O)N[C@@H](CC(=O)O)c2ccc(-c3ccc(OCCOCCOCCOCCOCCN=[N+]=[N-])c4ccccc34)cc2)C(=O)OC(C)(C)C)n1]

To a solution of compound 1 (0.0518 g) in 1:1 THF/water was added LiOH (0.0040 g) at room temperature under normal atmosphere. The reaction was stirred at rt until full conversion was observed by LC-MS. After 2 h, the reaction mixture was acidifed with 6 N HCl to a pH of −3. The product was extracted with EtOAc and then 20% CF₃CH₂OH/DCM (3×8 mL). The combined organic phase was dried over Na₂SO₄, filtered, and concentrated, providing a clear colorless oil. Yield: 0.0493 g (96.6%.) LC-MS: calculated [M+H]+ 916.44 m/z, observed 916.95 m/z.

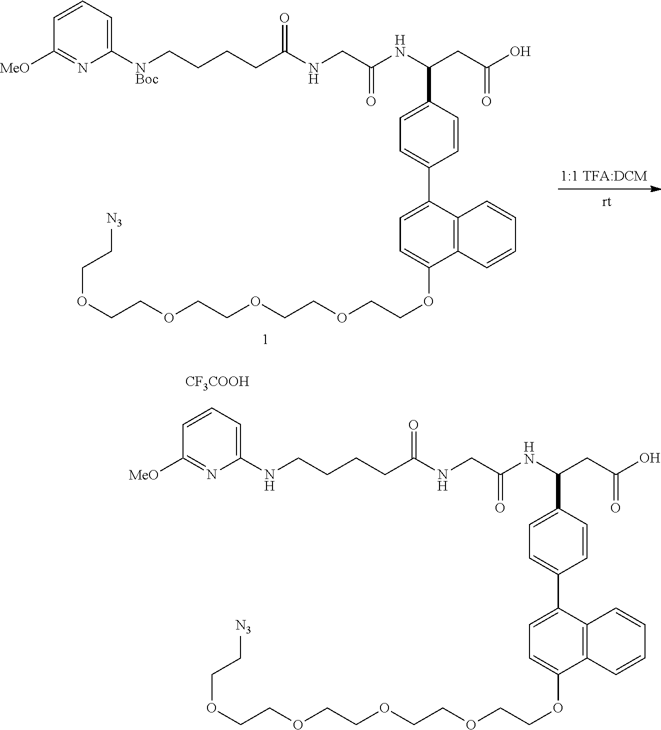 [SMILES: COc1cccc(N(CCCCC(=O)NCC(=O)N[C@@H](CC(=O)O)c2ccc(-c3ccc(OCCOCCOCCOCCOCCN=[N+]=[N-])c4ccccc34)cc2)C(=O)OC(C)(C)C)n1 ; "CF3COOH" ; COc1cccc(NCCCCC(=O)NCC(=O)N[C@@H](CC(=O)O)c2ccc(-c3ccc(OCCOCCOCCOCCOCCN=[N+]=[N-])c4ccccc34)cc2)n1]

To a solution of compound 1 (0.0493 g) in DCM was added TFA (0.124 mL) at room temperature. The reaction was stirred overnight under ambient conditions until full conversion was observed by LC-MS. The reaction mixture was azeotroped with PhMe and concentrated under vacuum to provide a light yellow oil. Yield: 0.0531 g (Yield: 106%.) LC-MS: calculated [M+H]+ 816.39 m/z, observed 816.66 m/z.

### Synthesis of Compound 58p, (S)-3-(4-(4-((14-azido-3,6,9,12-tetraoxatetradecyl)oxy)naphthalen-1-yl)phenyl)-3-(2-(5-((4-chloropyridin-2-yl)amino)pentanamido)acetamido)propanoic acid

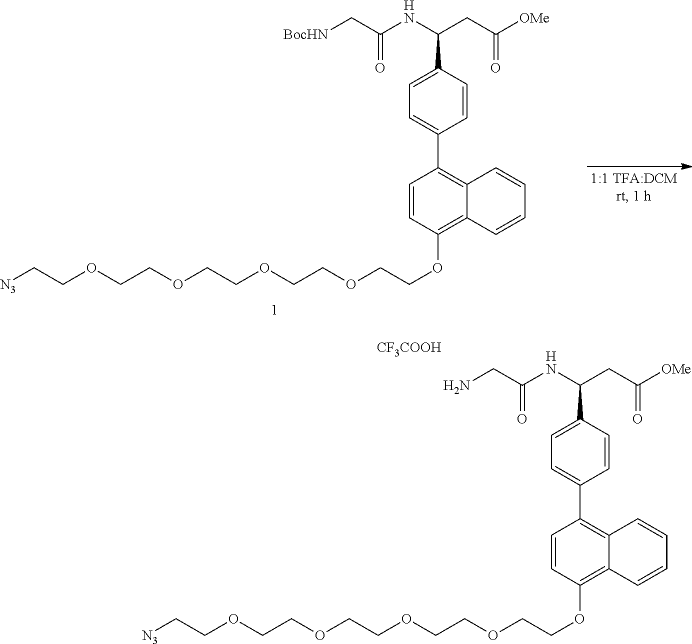 [SMILES: COC(=O)C[C@H](NC(=O)CNC(=O)OC(C)(C)C)c1ccc(-c2ccc(OCCOCCOCCOCCOCCN=[N+]=[N-])c3ccccc23)cc1 ; COC(=O)C[C@H](NC(=O)CN)c1ccc(-c2ccc(OCCOCCOCCOCCOCCN=[N+]=[N-])c3ccccc23)cc1 ; "CF3COOH"]

To a solution of compound 1 (0.244 g) in DCM was added TFA (1.15 g) at room temperature. The reaction was stirred under ambient conditions. After 1 h, full conversion was confirmed via LC-MS. The reaction mixture was concentrated under vacuum. No isolation was necessary. Concentration provided a yellow oil. Yield: 0.281 g (113%.) LC-MS: calculated [M+H]+ 624.30 m/z, observed 624.56 m/z.

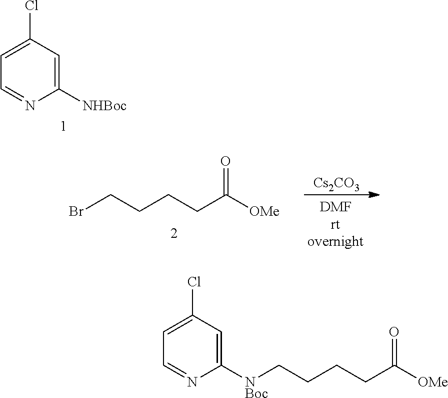

To a solution of compound 1 (0.300 g) in DMF was added Cs₂CO₃ (0.512 g) at room temperature. Compound 2 (0.269 g) was then added slowly dropwise. Reaction was stirred overnight. Approx. full conversion to desired product by LC-MS was then confirmed. The reaction mixture was quenched with NaHCO₃(10 mL). The product was extracted with EtOAc (3×8 mL) and then washed with water (3×8 mL) and brine (8 mL). The combined organic phase was dried over Na₂SO₄, filtered, and concentrated. The residue was purified by CombiFlash® using silica gel as the stationary phase with a gradient of hex to EtOAc (0-60%), in which product eluted at 7.5% B. The product was concentrated under vacuum to provide a clear and colorless oil. Yield: 0.311 g (69.2%.) LC-MS: calculated [M+H]+ 343.13 m/z, observed 343.08 m/z.

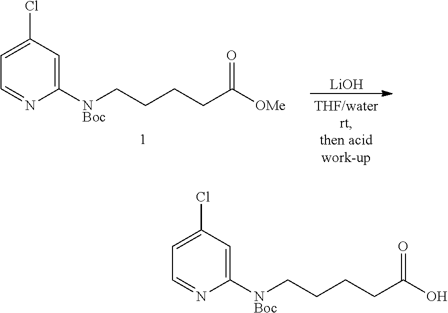 [SMILES: COC(=O)CCCCN(C(=O)OC(C)(C)C)c1cc(Cl)ccn1 ; "LiOH" ; CC(C)(C)OC(=O)N(CCCCC(=O)O)c1cc(Cl)ccn1]

To a solution of compound 1 (0.311 g) in 1:1 THF/water was added LiOH (0.0652 g) at room temperature under normal atmosphere. The reaction was stirred at room temperature until full conversion was observed by LC-MS. After 1 h, the reaction mixture was acidifed with 6 N HCl to a pH of −3. The product was extracted with EtOAc (3×8 mL). The combined organic phase was dried over Na₂SO₄, filtered, and concentrated, providing a clear, colorless oil. Yield: 0.311 g (104%.) LC-MS: calculated [M+H]+ 329.12 m/z, observed 329.31 m/z.

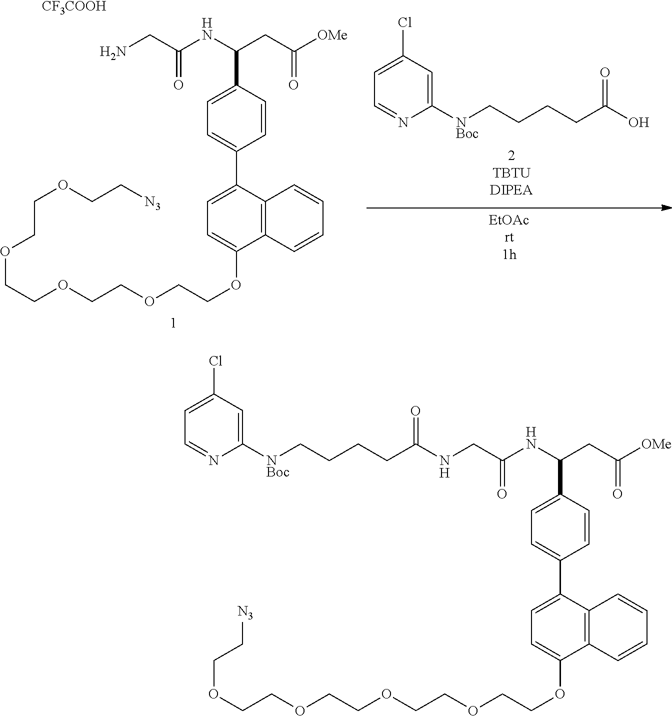 [SMILES: "CF3COOH" ; COC(=O)C[C@H](NC(=O)CN)c1ccc(-c2ccc(OCCOCCOCCOCCOCCN=[N+]=[N-])c3ccccc23)cc1 ; CC(C)(C)OC(=O)N(CCCCC(=O)O)c1cc(Cl)ccn1 ; COC(=O)C[C@H](NC(=O)CNC(=O)CCCCN(C(=O)OC(C)(C)C)c1cc(Cl)ccn1)c1ccc(-c2ccc(OCCOCCOCCOCCOCCN=[N+]=[N-])c3ccccc23)cc1]

To a solution of compounds 1 (0.0700 g) and 2 (0.0328 g) in EtOAc was added TBTU (0.0366 g) and then DIPEA (0.066 mL) under ambient conditions. The reaction was stirred for 1 h until full conversion was observed by LC-MS. The reaction mixture was then quenched with NaHCO₃(8 mL). The product was extracted with EtOAc (3×5 mL) and 20% CF₃CH₂OH/DCM (3×5 mL) and then washed with water (3×5 mL) and brine (5 mL). The combined organic phase was dried over Na₂SO₄, filtered, and concentrated. The residue was purified by CombiFlash® using silica gel as the stationary phase with a gradient of DCM to 20% MeOH in DCM (0-100%), in which product eluted at 21% B. The product was concentrated under vacuum to provide a clear colorless oil. Yield: 0.0790 g (89.1%.) LC-MS: calculated [M+H]+ 934.40 m/z, observed 935.13 m/z.

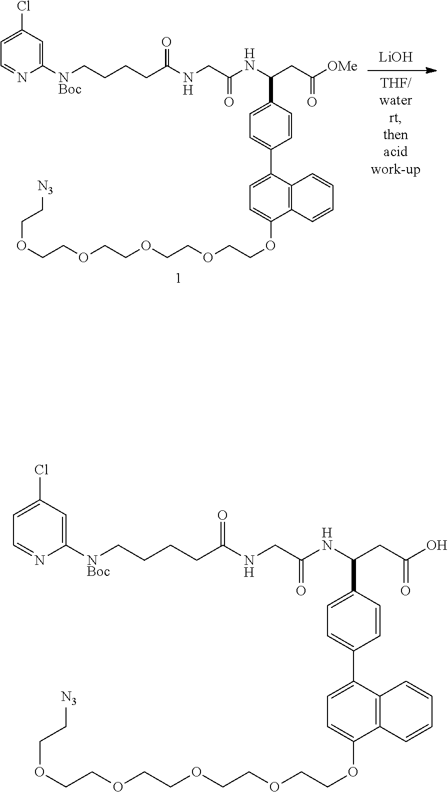 [SMILES: COC(=O)C[C@H](NC(=O)CNC(=O)CCCCN(C(=O)OC(C)(C)C)c1cc(Cl)ccn1)c1ccc(-c2ccc(OCCOCCOCCOCCOCCN=[N+]=[N-])c3ccccc23)cc1 ; "LiOH" ; "THF/waterrt, then acidwork-up" ; CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(Cl)ccn1]

To a solution of compound 1 (0.0790 g) in 1:1 THF/water was added LiOH (0.0061 g) at room temperature under normal atmosphere. The reaction was stirred at room temperature until full conversion was observed by LC-MS. After 1 h, the reaction mixture was acidifed with 6 N HCl to a pH of −3-4. The product was extracted with 20% CF₃CH₂OH/DCM (3×8 mL). The combined organic phase was dried over Na₂SO₄, filtered, and concentrated, providing a clear and colorless oil. Yield: 0.0776 g (99.7%.) LC-MS: calculated [M+H]+ 920.39 m/z, observed 921.00 m/z.

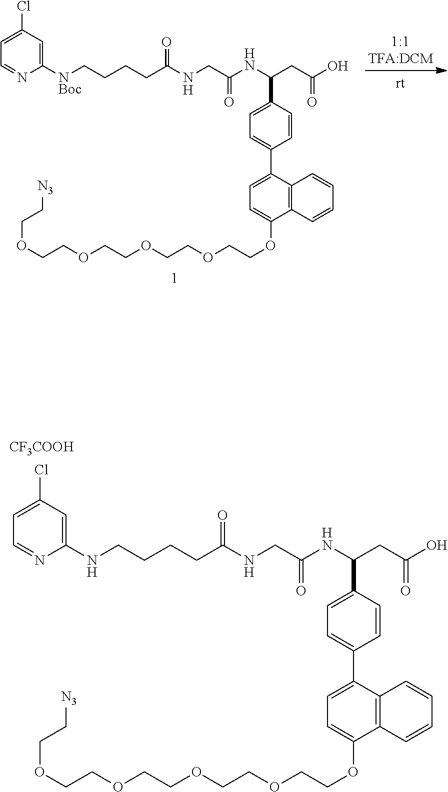 [SMILES: CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(Cl)ccn1 ; "1:1TFA:DCM" ; "rt" ; "CF3COOH" ; [N-]=[N+]=NCCOCCOCCOCCOCCOc1ccc(-c2ccc([C@H](CC(=O)O)NC(=O)CNC(=O)CCCCNc3cc(Cl)ccn3)cc2)c2ccccc12]

To a solution of compound 1 (0.0776 g) in DCM was added TFA at room temperature. The reaction was stirred under ambient conditions. Reaction was stirred overnight until full conversion was confirmed via LC-MS. The reaction mixture was azeotroped with PhMe and concentrated under vacuum. No isolation was necessary. Concentration provided a yellow oil. Yield: 0.0590 g (74.9%.) LC-MS: calculated [M+H]+ 820.34 m/z, observed 820.99 m/z.

### Synthesis of Compound 59p, (S)-3-(4-(4-((14-azido-3,6,9,12-tetraoxatetradecyl)oxy)naphthalen-1-yl)phenyl)-3-(2-(5-((4-fluoropyridin-2-yl)amino)pentanamido)acetamido)propanoic acid

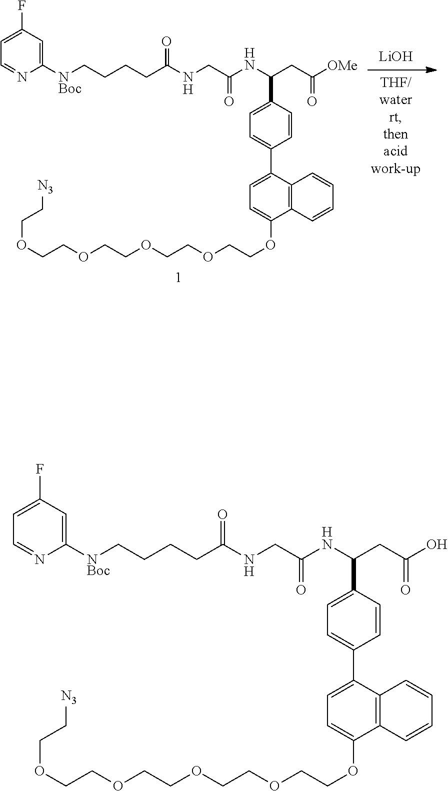 [SMILES: COC(=O)C[C@H](NC(=O)CNC(=O)CCCCN(C(=O)OC(C)(C)C)c1cc(F)ccn1)c1ccc(-c2ccc(OCCOCCOCCOCCOCCN=[N+]=[N-])c3ccccc23)cc1 ; "LiOH" ; "THF/waterrt, then acidwork-up" ; CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(F)ccn1]

To a solution of compound 1 (0.121 g) in 1:1 THF/water was added LiOH (0.0095 g) at room temperature under normal atmosphere. The reaction was stirred at room temperature until full conversion was observed by LC-MS. After 1 h, the reaction mixture was acidifed with 6 N HCl to a pH of −3-4. The product was extracted with 20% CF₃CH₂OH/DCM (3×8 mL). The combined organic phase was dried over Na₂SO₄, filtered, and concentrated, providing a cream white solid. Yield: 0.0868 g (72.8%.) LC-MS: calculated [M+H]+ 904.42 m/z, observed 905.07 m/z.

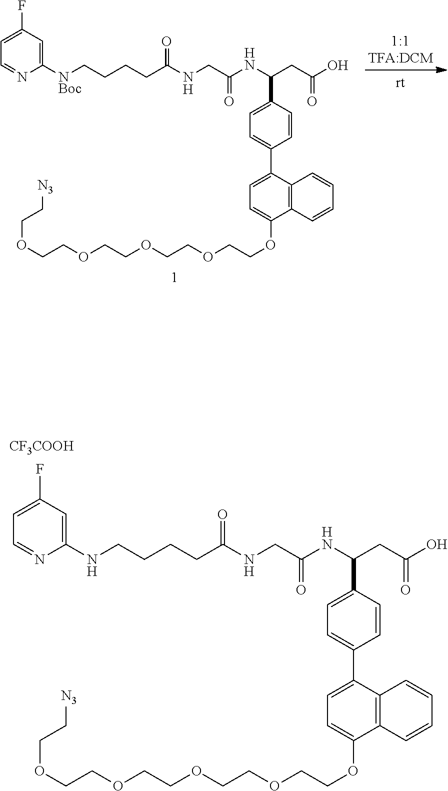 [SMILES: CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1cc(F)ccn1 ; "1:1TFA:DCM" ; "rt" ; "CF3COOH" ; [N-]=[N+]=NCCOCCOCCOCCOCCOc1ccc(-c2ccc([C@H](CC(=O)O)NC(=O)CNC(=O)CCCCNc3cc(F)ccn3)cc2)c2ccccc12]

To a solution of compound 1 (0.868 g) in DCM was added TFA (0.220 mL) at room temperature. The reaction was stirred under ambient conditions. The reaction was stirred overnight until full conversion was confirmed via LC-MS. The reaction mixture was azeotroped with PhMe and concentrated under vacuum. No isolation was necessary. Concentration provided a yellow oil. Yield: 0.0380 g (43.1%.) LC-MS: calculated [M+H]+ 804.37 m/z, observed 804.78 m/z.

### Synthesis of Compound 60p, (S)-3-(4-(4-((14-azido-3,6,9,12-tetraoxatetradecyl)oxy)naphthalen-1-yl)phenyl)-3-(2-(5-(pyridin-2-ylamino)pentanamido)acetamido)propanoic acid

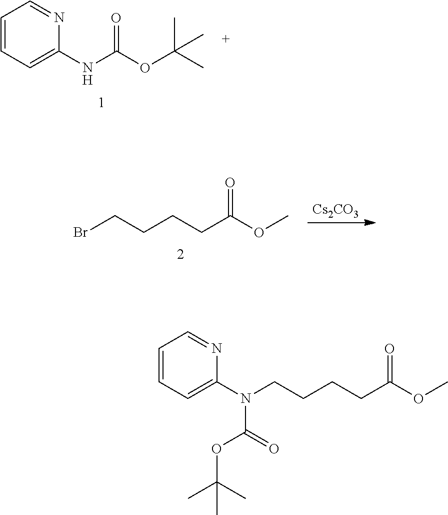

To a solution of compound 1 (211 mg, 1.086 mmol, 1.0 equiv.), and cesium carbonate (530 mg, 1.629 mmol, 1.5 equiv.) in anhydrous DMF (2 mL) was added compound 2 (0.187 mL, 1.303 mmol, 1.2 equiv.) at room temperature. The reaction was kept at room temperature for 72 hrs. The reaction was quenched with water (5 mL). The aqueous phase was extracted with ethyl acetate (3×5 mL). The organic phase was combined, dried over Na₂SO₄, and concentrated. The product was purified by CombiFlash® and eluted with 10-15% ethyl acetate in hexane. LC-MS: calculated [M+H]+ 309.17, found 309.42.

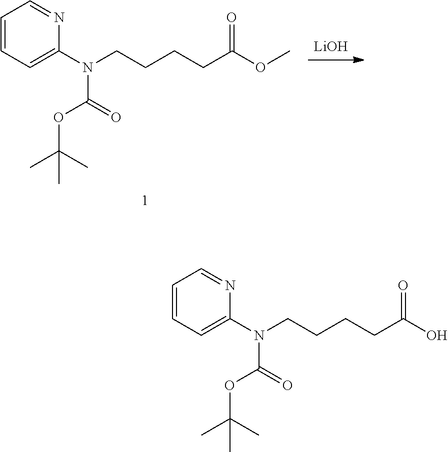

To a solution of compound 1 (348 mg, 1.128 mmol, 1.0 equiv.) in THE (5 mL) and water (5 mL) was added lithium hydroxide (81 mg, 3.385 mmol, 3.0 equiv.) at room temperature. The reaction was kept at room temperature for 1 hr. The reaction was quenched with HCl solution and the pH was adjusted to 3.0. The aqueous phase was extracted with ethyl acetate (3×10 mL). The organic phase was combined, dried over Na₂SO₄, and concentrated. The product was used directly without further purification. LC-MS: calculated [M+H]+ 295.16, found 295.38.

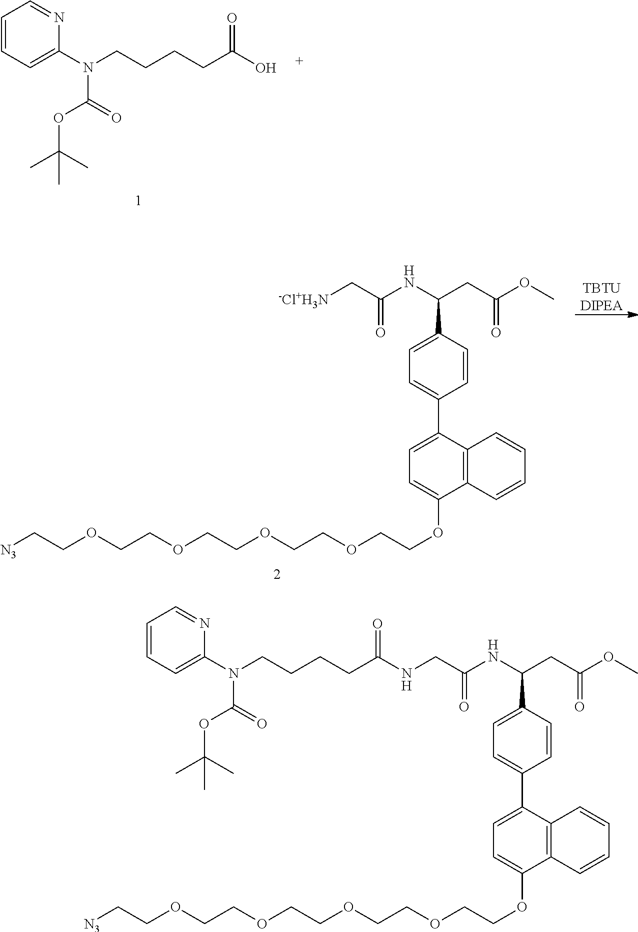

To a solution of compound 1 (44 mg, 0.149 mmol, 1.0 equiv.), compound 2 (108 mg, 0.164 mmol, 1.1 equiv.) and diisopropylethylamine (0.078 mL, 0.448 mmol, 3.0 equiv.) in anhydrous DMF (1 mL) was added TBTU (57 mg, 0.179 mmol, 1.2 equiv.) at room temperature. The reaction was kept at room temperature for 2 hrs. The reaction was quenched with saturated NaHCO₃(5 mL), and the aqueous phase was extracted with ethyl acetate (3×5 mL). The organic phase was combined, dried over Na₂SO₄, and concentrated. The product was purified by CombiFlash® and eluted with 3-5% methanol in dichloromethane. LC-MS: calculated [M+H]+ 900.44, found 901.19.

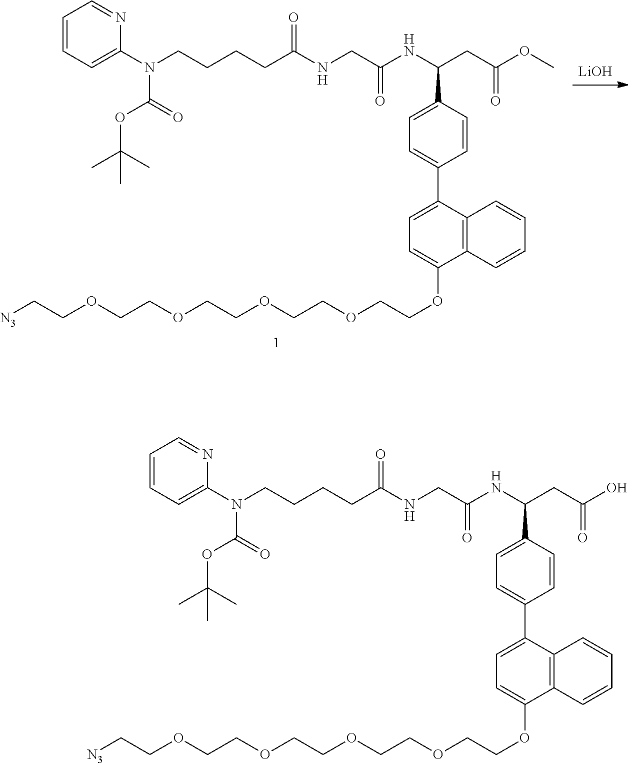

To a solution of compound 1 (110 mg, 0.122 mmol, 1.0 equiv.) in THE (3 mL) and water (3 mL) was added lithium hydroxide (9 mg, 0.366 mmol, 3.0 equiv.) at room temperature. The reaction was kept at room temperature for 3 hrs. The reaction was quenched with HCl solution and the pH was adjusted to 3.0. The aqueous phase was extracted with ethyl acetate (3×5 mL). The organic phase was combined, dried over Na₂SO₄, and concentrated. The product was used directly without further purification. LC-MS: calculated [M+H]+ 886.43, found 886.97.

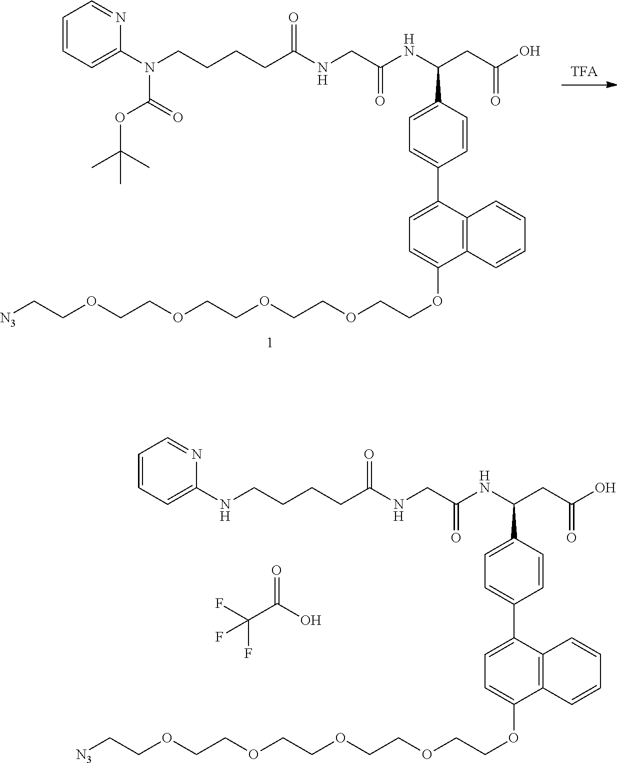 [SMILES: CC(C)(C)OC(=O)N(CCCCC(=O)NCC(=O)N[C@@H](CC(=O)O)c1ccc(-c2ccc(OCCOCCOCCOCCOCCN=[N+]=[N-])c3ccccc23)cc1)c1ccccn1 ; [N-]=[N+]=NCCOCCOCCOCCOCCOc1ccc(-c2ccc([C@H](CC(=O)O)NC(=O)CNC(=O)CCCCNc3ccccn3)cc2)c2ccccc12 ; O=C(O)C(F)(F)F ; "TFA"]

To a solution of compound 1 (108 mg, 0.121 mmol, 1.0 equiv.) in dichloromethane (2 mL) was added trifluoroacetic acid (2 mL) at room temperature. The reaction was kept at room temperature for 2 hrs. The solvent was removed. The product was used directly without further purification. LC-MS: calculated [M+H]+ 786.37, found 787.05.

### Example 4. Synthesis of PK/PD Modulators

Some of the PK/PD modulators of Table 5 were purchased from commercial suppliers and are indicated as such in Table 5. The following procedures were used to prepare the remaining PK/PD modulators used in later examples.

### Bis(PEG47+C22)

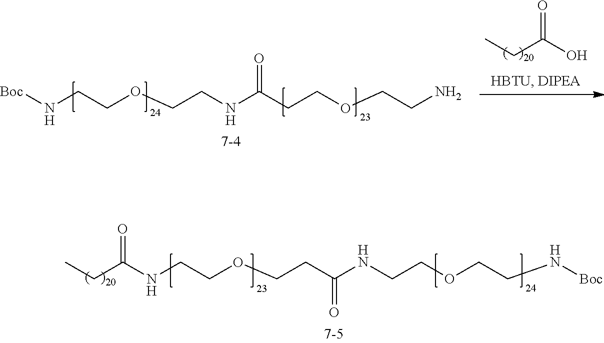

Solid TBTU (1.68 g, 5.22 mmol) was added to a solution of behenic acid (1.486 g, 4.36 mmol), Boc-protected Peg-amine 7-4 (Quanta Biodesign Limited, 10 g, 4.35 mmol), and DIPEA (2.27 mL, 13.03 mmol). The reaction mixture was sonicated to dissolve solids and stirred for 16 h at RT. Water (3 mL) was added, the solvent was removed in high vacuo, the residue was dissolved in chloroform (300 mL) and washed with NaHCO₃(2×75 mL), and brine (50 mL). The product was dried (Na₂SO₄), concentrated in vacuo, and purified on Combiflash® using the system DCM: 20% MeOH in DCM, gradient 0-80%, 25 min. Yield 10 g (88%). Calculated MW 2623.72, (M+2×18)/2=1329.86, (M+3H)/3=875.57 Found: MS (ES, pos): 1330.58 [M+2NH₄]²⁺, 875.93 [M+3H]³⁺.

### C18

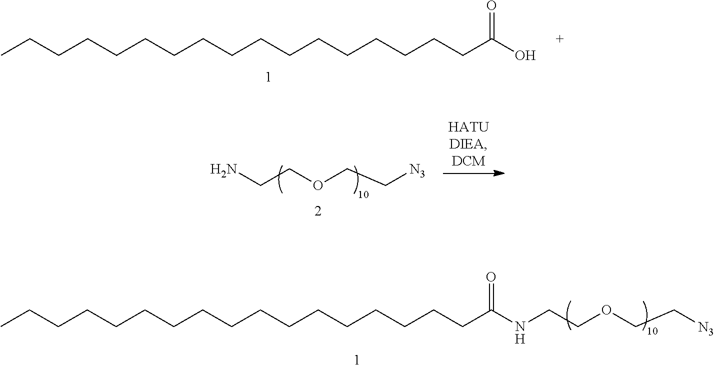

Compound 1 (Sigma S4751) (0.125 g) was dissolved in DCM (2.0 mL). Then HATU (0.249 g) and DIEA (0.263 mL) were added to the mixture. The reaction was allowed to stir for 15 minutes. Then 0.265 g of compound 2 (BroadPharm® BP-22226) was added. The reaction was allowed to stir for 1 hour.

The reaction was then diluted with DCM (40 mL) then washed with H₂O (2×7 mL), dried over Na₂SO₄, filtered and concentrated by rotary evaporator. The organic layer was brought up in 2 mL of DCM and purified on column (Combi-Gumbi in DCM: DCM with 20% McOH, RediSep-RF Gold column; 0-40% mobile phase B over 30 minutes. The fractions containing product were collected and concentrated on rotary evaporator and high vacuum. Yield 223 mg (56%.)

C22-PEG5K-Mal

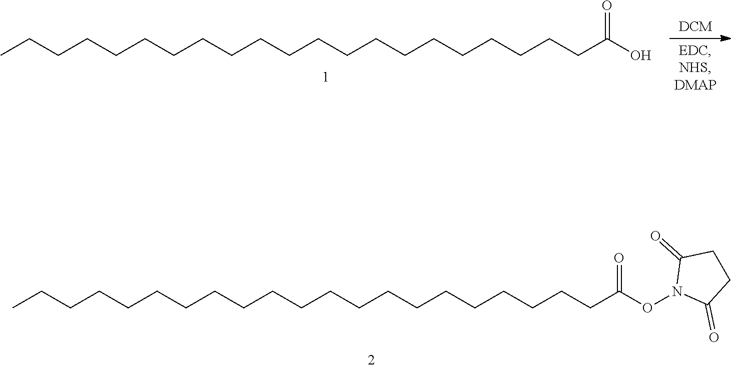

Compound 1 (Sigma-Aldrich® 216941) (0.300 g) was dissolved in 4.5 mL of DCM. Then EDC (Oakwood Chemical 024810) (0.211 g) was added to the solution. Then NHS (Sigma-Aldrich® 130672) (0.203 g) was added to the mixture. Finally DMAP (Sigma-Aldrich® 107700) (0.0215 g) was added. The reaction was allowed to stir overnight. The solution was diluted with 40 mL of DCM, and washed with acidic H₂O (3×7 mL), dried with Na₂SO₄, filtered and concentrated on rotary evaporator. The concentrated product was dry loaded (3 mL of silica) onto 12 G Redi-Sep Gold Rf column in (mobile phase A: mobile phase B) Hex: EtOAc 0≥50% over 25 minutes. The fractions containing product were collected and concentrated on rotary evaporator. Yield 283 g (73%.)

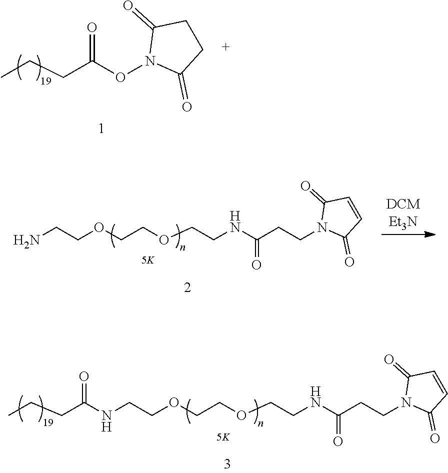

Compound 2 (Creative PEGWorks PHB-942) (0.100 g) was dissolved in 2 mL of DCM. Compound 1 (0.0438 g) was then added. Then 0.042 mL of Et3N was added to the mixture. The reaction was allowed to stir for 2 hours. The reaction mixture was concentrated on high vacuum. The concentrate was then brought up in 1 mL of DCM and loaded onto 4 G Redi-Sep Gold Rf column in DCM: DCM with 20% MeOH 0≥100% over 20 minutes. The fractions containing product were collected and concentrated on rotary evaporator. Yield 41 mg (38%).

### PEG48+C22

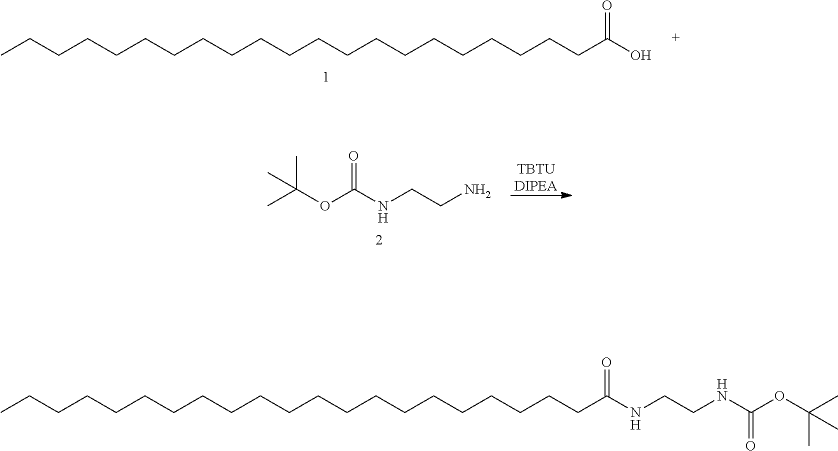 [SMILES: CCCCCCCCCCCCCCCCCCCCCC(=O)O ; CC(C)(C)OC(=O)NCCN ; "TBTUDIPEA" ; CCCCCCCCCCCCCCCCCCCCCC(=O)NCCNC(=O)OC(C)(C)C]

To a solution of compound 1 (350 mg, 1.027 mmol, 1.0 equiv.), compound 2 (181 mg, 1.130 mmol, 1.1 equiv.) and diisopropylethylamine (0.537 mL, 3.082 mmol, 3.0 equiv.) in anhydrous DMF (3 mL) was added TBTU (396 mg, 1.233 mmol, 1.2 equiv.) at room temperature. The reaction was kept at room temperature for 2 hrs. The reaction was quenched with saturated NaHCO₃ aqueous solution (20 mL) and the aqueous was extracted with dicholoromethane (3×10 mL). The organic phase was combined, dried over anhydrous Na₂SO₄, and concentrated. The product was purified by CombiFlash® and was eluted with 4-5% methanol in dichloromethane. LC-MS: calculated [M+H]+ 483.44, found 483.67.

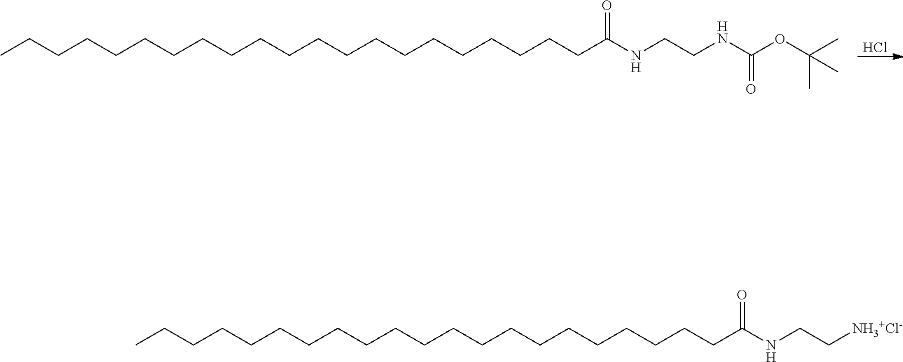

To a solution of compound 1 (290 mg, 0.600 mmol, 1.0 equiv.) in anhydrous 1,4-dioxane (1 mL) was added HCl solution in dioxane (0.751 mL, 3.003 mmol, 5.0 equiv.) at room temperature. The reaction was kept at room temperature for 3 hrs and the solvent was concentrated. The product was used directly without further purification. LC-MS: calculated [M+H]+ 383.39, found 383.57.

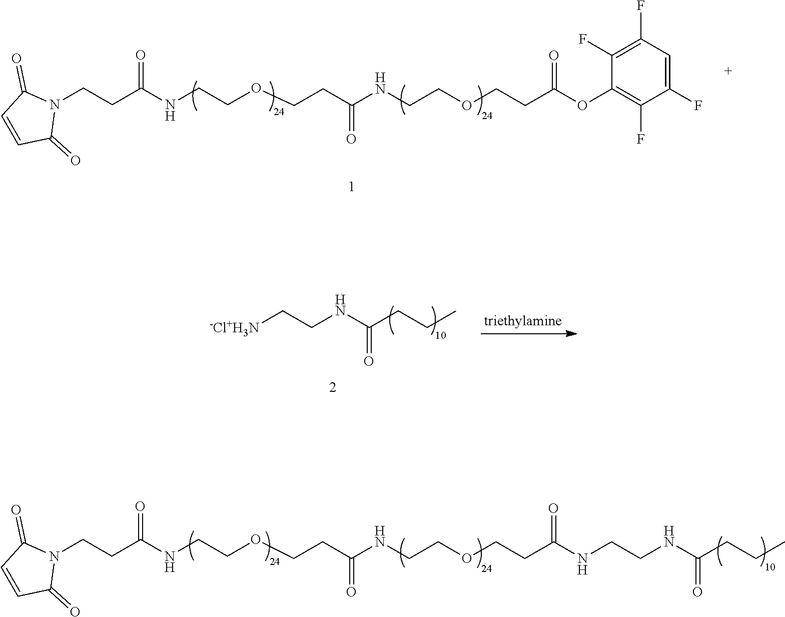

To a solution of compound 1 (83 mg, 0.0322 mmol, 1.0 equiv.) and compound 2 (13.5 mg, 0.322 mmol, 1.0 equiv.) in anhydrous DMF (2 mL) was added triethylamine (0.014 mL, 0.0967 mmol, 3.0 equiv.) at room temperature. The reaction was kept at room temperature for 3 hrs and the solvent was concentrated. The product was separated by CombiFlash and was eluted with 10-15% methanol in dicholoromethane. LC-MS: calculated [M+4H]+/4 698.18, found 698.49, calculated [M+3H]+/3 930.58, found 930.61.

### PEG48+C18

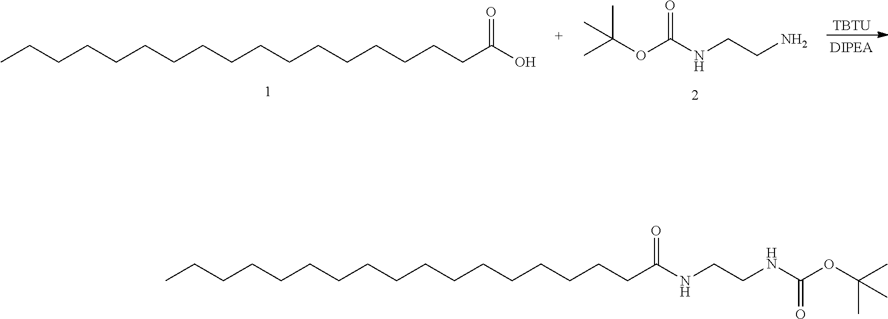

To a solution of compound 1 (1437 mg, 5.051 mmol, 1.0 equiv.), compound 2 (890 mg, 5.556 mmol, 1.1 equiv.) and diisopropylethylamine (2.639 mL, 15.154 mmol, 3.0 equiv.) in anhydrous DMF (10 mL) was added TBTU (1946 mg, 6.061 mmol, 1.2 equiv.) at room temperature. The reaction was kept at room temperature for 2 hrs. The reaction was quenched with saturated NaHCO₃ aqueous solution (20 mL) and the aqueous was extracted with dicholoromethane (3×10 mL). The organic phase was combined, dried over anhydrous Na₂SO₄, and concentrated. The product was purified by CombiFlash and was eluted with 4-5% methanol in dichloromethane. LC-MS: calculated [M+H]+ 427.38, found 427.74.

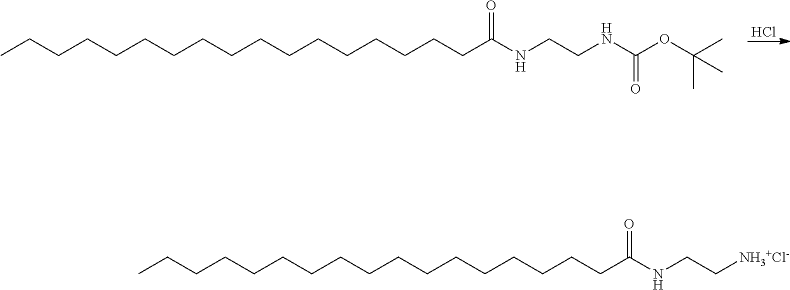

To a solution of compound 1 (445 mg, 1.042 mmol, 1.0 equiv.) in anhydrous 1,4-dioxane (1 mL) was added HCl solution in dioxane (1.304 mL, 5.214 mmol, 5.0 equiv.) at room temperature. The reaction was kept at room temperature for 3 hrs and the solvent was concentrated. The product was used directly without further purification. LC-MS: calculated [M+H]+ 327.33, found 327.48.

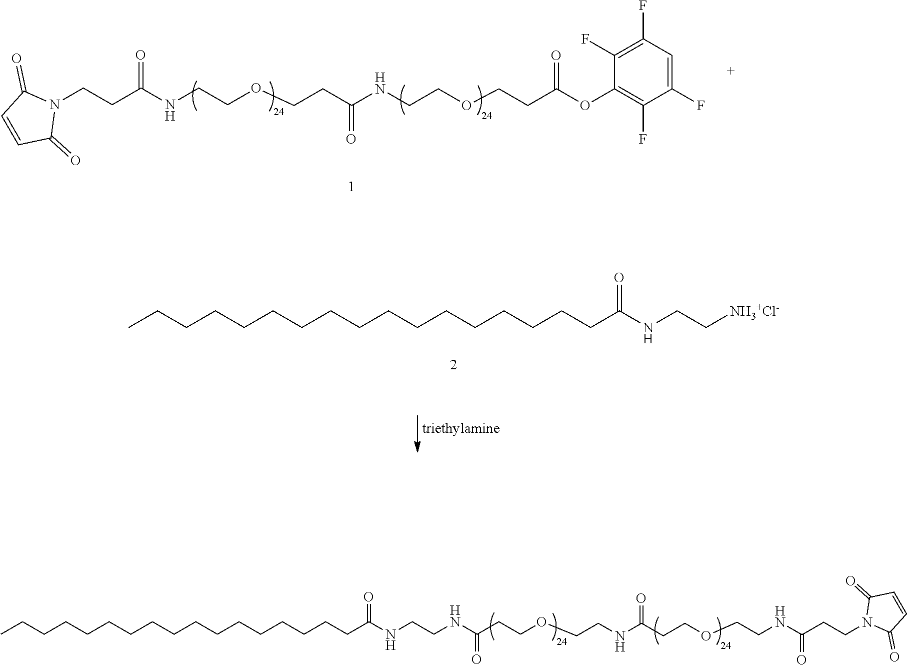

To a solution of compound 1 (90 mg, 0.035 mmol, 1.0 equiv.) and compound 2 (13.3 mg, 0.0367 mmol, 1.05 equiv.) in anhydrous DCM (2 mL) was added triethylamine (0.015 mL, 0.104 mmol, 3.0 equiv.) at room temperature. The reaction was kept at room temperature for 1 hr and the solvent was concentrated. The product was separated by CombiFlash and was eluted with 12-18% methanol in dicholoromethane. LC-MS: calculated [M+3H]+/3 911.90, found 912.65, [M+4H]+/4 684.17, found 685.21.

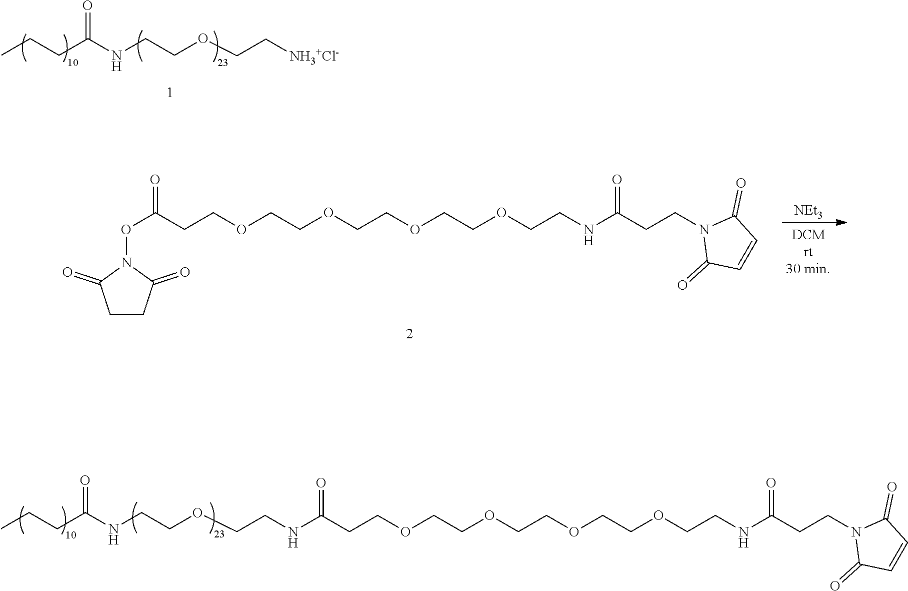

To a solution of compound 1 (0.0700 g) in DCM was added 2 (0.0251 g) and NEt₃ (0.0148 g) at room temperature. The mixture was stirred for 0.5 h until full conversion was confirmed by LC-MS. The reaction mixture was concentrated immediately for isolation. The residue was purified by CombiFlash® using silica gel as the stationary phase and was eluted with a gradient of DCM to 20% MeOH in DCM (0-100% B). Product eluted at 60% B. Concentration provided a white solid. LC-MS: calculated [M+H]+ 1794.16 m/z, observed 898.01 (+2/2) m/z. Yield: 0.0784 g (89.4%.)

### Bis(PEG₂₃+C₁₄)

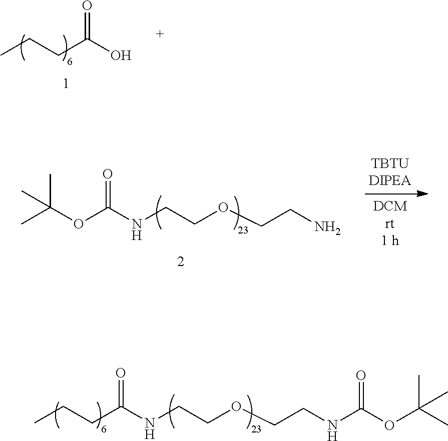

To a solution of compounds 1 (0.0430 g) and 2 (0.221 g) in DCM was added TBTU (0.0725 g) and then DIPEA (0.098 mL) under ambient conditions. The reaction was stirred for 1 h until full conversion was observed by LC-MS. The reaction mixture was then immediately concentrated for isolation. The residue was purified by CombiFlash® using silica gel as the stationary phase with a gradient of DCM to 20% MeOH in DCM (0-100%), in which product eluted at 31% B. The product was concentrated under vacuum to provide a white solid. LC-MS: calculated [M+H]+ 1383.92 m/z, observed 693.02 (+2/2) m/z. Yield: 0.253 g (97.0%.)

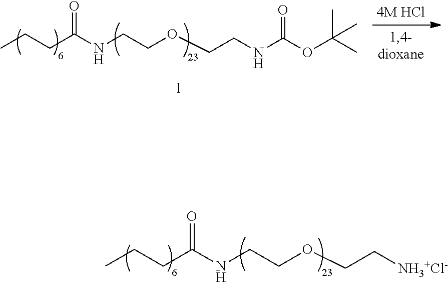

To compound 1 (0.253 g) was added 4 M HCl (2.74 mL) in 1,4-dioxane at rt. The reaction was stirred for 1 h under ambient conditions until full conversion was observed by LC-MS. The reaction mixture was concentrated under vacuum to provide a white solid. No isolation necessary. LC-MS: calculated [M+H]+ 1319.85 m/z, observed 642.97 (+2/2) m/z. Yield: 0.241 g (99.7% .)

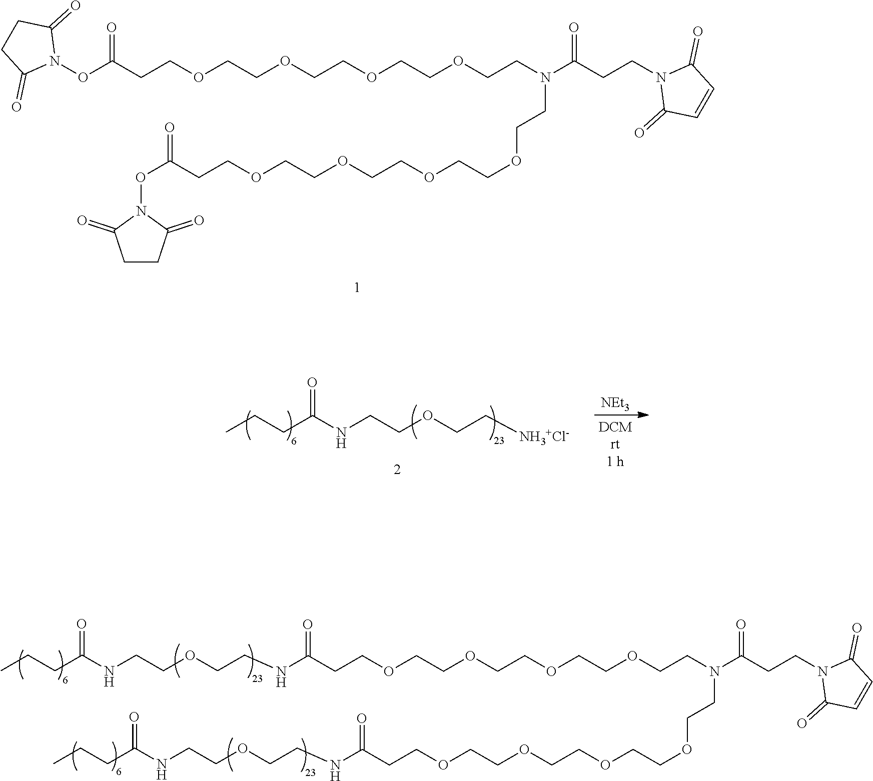

To a solution of compound 2 (0.169 g) in DCM was added 1 (0.0500) and then NEt₃ (0.049 mL) at rt. The mixture was stirred overnight until full conversion was confirmed by LC-MS. The reaction mixture was concentrated immediately for isolation. The residue was purified by CombiFlash® using silica gel as the stationary phase and was eluted with a gradient of DCM to 20% MeOH in DCM (0-100% B). Product eluted at 95% B. Concentration provided a white solid. LC-MS: calculated [M+H]+ 3195.02 m/z, observed 800.43 (+4/4) m/z. Yield 0.0674 (36.2%.)

### Bis(PEG23+C18)

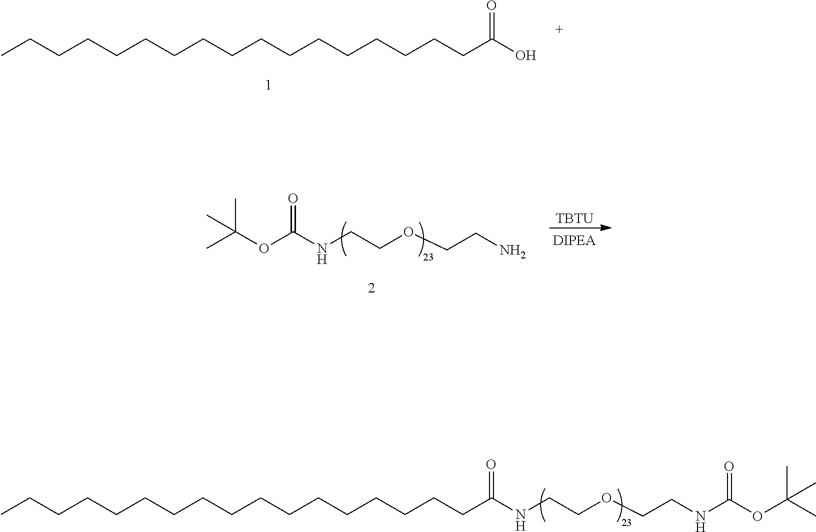

To a solution of compound 1 (130 mg, 0.457 mmol, 1.0 equiv.), compound 2 (536 mg, 0.457 mmol, 1.0 equiv.) and diisopropylethylamine (0.239 mL, 1.370 mmol, 3.0 equiv.) in anhydrous DMF (3 mL) was added TBTU (176 mg, 0.548 mmol, 1.2 equiv.) at room temperature. The reaction was kept at room temperature for 2 hrs. The reaction was quenched with saturated NaHCO₃ aqueous solution (5 mL) and the aqueous was extracted with ethyl acetate (6×5 mL). The organic phase was combined, dried over anhydrous Na₂SO₄, and concentrated. The product was purified by CombiFlash® and was eluted with 4-8% methanol in dichloromethane. LC-MS: calculated [M+H]+ 1439.99, found 1440.53.

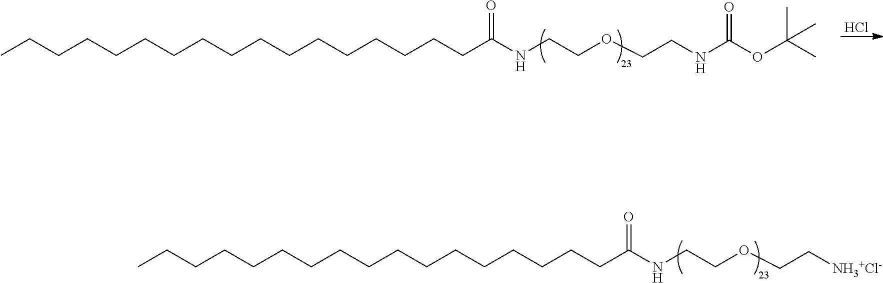

To a solution of compound 1 (445 mg, 0.309 mmol, 1.0 equiv.) in anhydrous 1,4-dioxane (1 mL) was added HCl solution in dioxane (0.386 mL, 1.545 mmol, 5.0 equiv.) at room temperature. The reaction was kept at room temperature for 3 hrs and the solvent was concentrated. The product was used directly without further purification. LC-MS: calculated [M+2H]+/2 670.46, found 670.93.

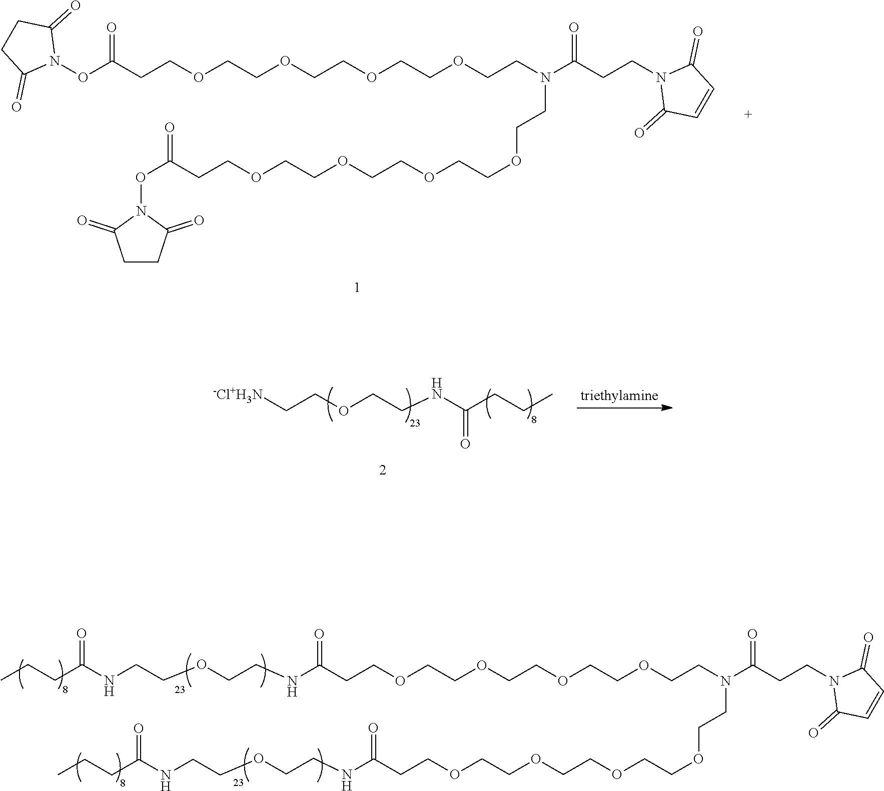

To a solution of compound 1 (100 mg, 0.116 mmol, 1.0 equiv.) and compound 2 (352 mg, 0.256 mmol, 2.2 equiv.) in anhydrous DMF (5 mL) was added triethylamine (0.082 mL, 0.582 mmol, 5.0 equiv.) at room temperature. The reaction was kept at room temperature for 3 hrs and the solvent was concentrated. The product was separated by CombiFlash and was eluted with 10-17% methanol in dicholoromethane. LC-MS: calculated [M+4H]+/4 827.53, found 828.16, calculated [M+3H]+/3 1103.05, found 1104.21.

### Bis(PEG23+C22)

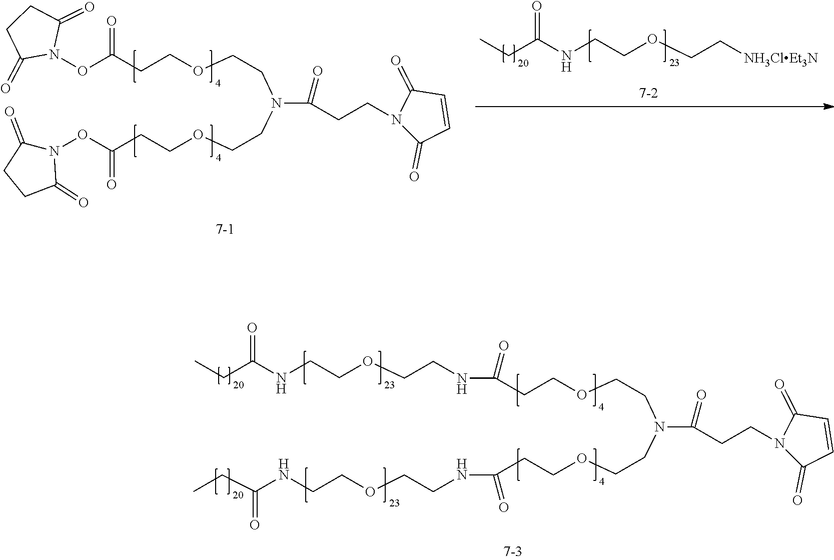

A solution of C22-Peg23-amine hydrochloride (7-2), (Quanta Biodesign Limited, 183 mg, 0.128 mmol) and bis-NHS ester 7-1, (BroadPharm, 50 mg, 0.058 mmol) in DMF (5 mL) were stirred at RT in presence of Et3N (50 uL, 0.35 mmol) for 3h. The reaction mixture was concentrated and dried in vacuo. The residual DMF was removed by co-evaporation of toluene on a rotavapor, and the product 7-3 was purified on combiflash using the system DCM: 20% MeOH in DCM, gradient 15-80%, 25 min. Yield 84 mg (45%). Calculated MW 3419.28, ½M=1709.64, (M+2×18)/2=1727.64, (M+18+2)/3=1146.42. Found: MS (ES, pos): 1727.73 [M+2NH₄]²⁺, 1146.94 [M+NH₄+2H]³⁺.

### Bis(PEG23+CLS)

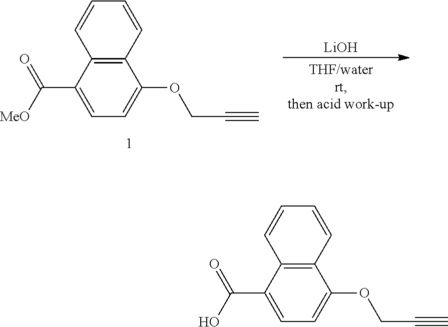 [SMILES: C#CCOc1ccc(C(=O)OC)c2ccccc12 ; "LiOH" ; C#CCOc1ccc(C(=O)O)c2ccccc12]

To a solution of compound 1 (0.158 g) in 1:1 THE/water was added LiOH (0.0473 g) at rt under normal atmosphere. The reaction was stirred at rt for 1 h and then heated to 50° C. and stirred overnight until full conversion was observed by LC-MS. The reaction mixture was acidifed with 6 N HCl to a pH of −3. The product was extracted with EtOAc (3×5 mL). The combined organic phase was dried over Na₂SO₄, filtered, and concentrated, providing a white solid. LC-MS: calculated [M+H]+ 227.06 m/z, observed 227.06 m/z. Yield: 152 mg (102%.)

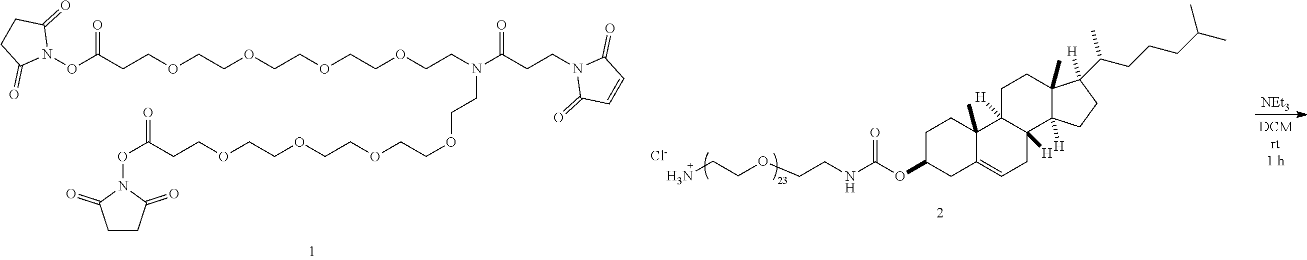

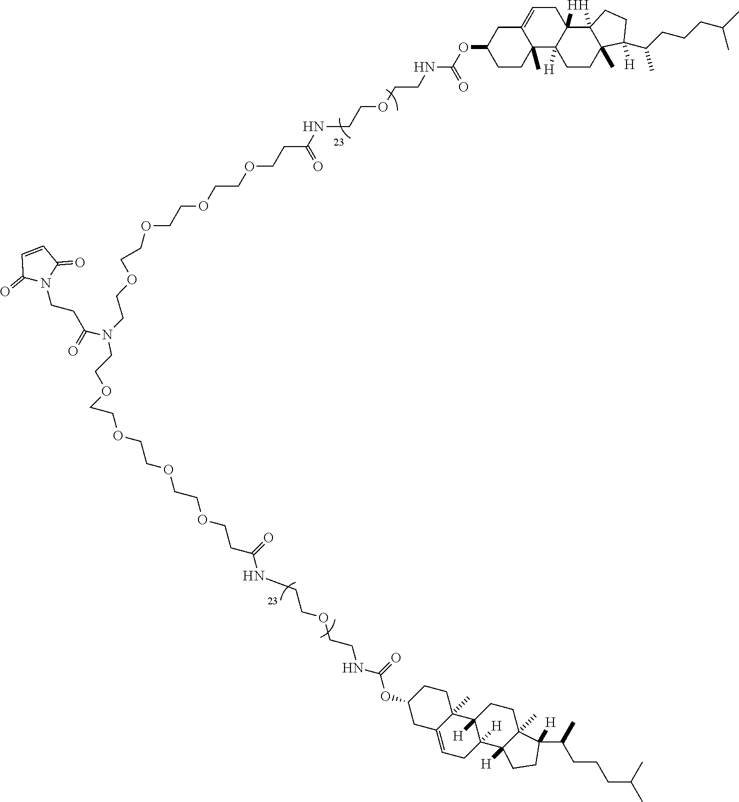

To a solution of compound 2 (0.0709 g) in DCM was added 1 (0.0200 g) and then NEt₃ (0.019 mL) at rt. The mixture was stirred until full conversion was confirmed by LC-MS. The reaction mixture was concentrated immediately for isolation. The residue was purified by CombiFlash® using silica gel as the stationary phase and was eluted with a gradient of DCM to 20% MeOH in DCM (0-100% B). Product eluted at 82% B. Concentration provided a white solid. LC-MS: calculated [M+H]+ 3599.30 m/z, observed 1200.23 (+3/3) m/z. Yield 0.0211 g (25.2%)

### Tris(PEG23+C22)

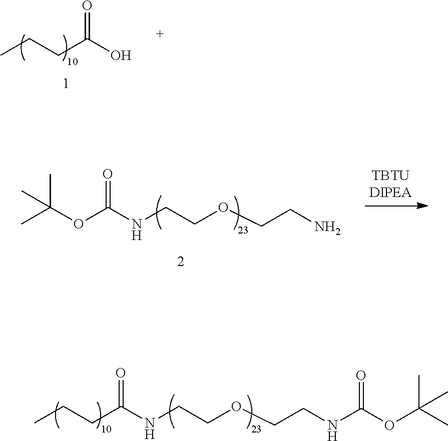

To a solution of compound 1 (290 mg, 0.851 mmol, 1.0 equiv.), compound 2 (999 mg, 0.851 mmol, 1.0 equiv.) and diisopropylethylamine (0.445 mL, 2.554 mmol, 3.0 equiv.) in anhydrous DMF (3 mL) was added TBTU (328 mg, 1.021 mmol, 1.2 equiv.) at room temperature. The reaction was kept at room temperature for 2 hrs. The reaction was quenched with saturated NaHCO₃ aqueous solution (10 mL) and the aqueous was extracted with dichloromethane (3×10 mL). The organic phase was combined, dried over anhydrous Na₂SO₄, and concentrated. The product was purified by CombiFlash and was eluted with 7-16% methanol in dichloromethane. LC-MS: calculated [M+H]+ 1496.05, found 1496.59.

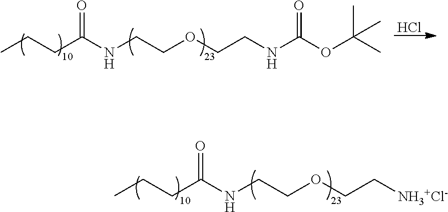

To a solution of compound 1 (642 mg, 0.429 mmol, 1.0 equiv.) in anhydrous 1,4-dioxane (0.5 mL) was added HCl solution in dioxane (2.146 mL, 8.582 mmol, 20 equiv.) at room temperature. The reaction was kept at room temperature for 30 min and the solvent was concentrated. The product was used directly without further purification. LC-MS: [M+H]+ calculated 1396.00, found 1396.60.

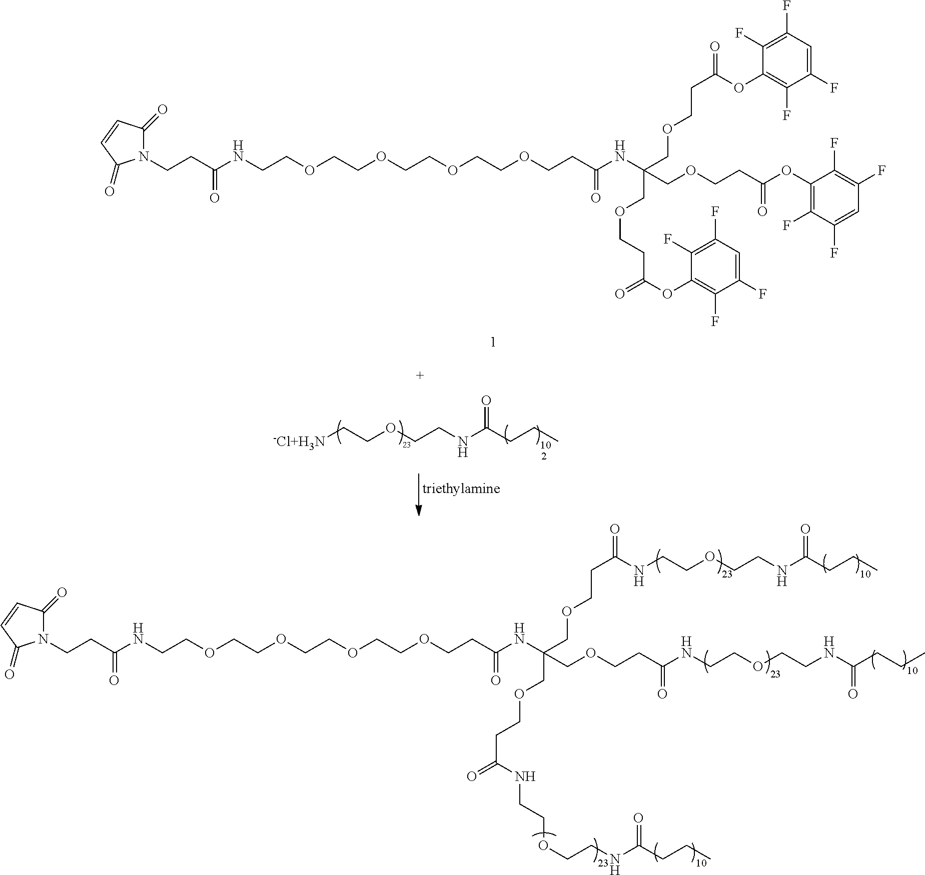

To a solution of compound 1 (24 mg, 0.0203 mmol, 1.0 equiv.) and compound 2 (94 mg, 0.062 mmol, 3.05 equiv.) in anhydrous DMF (2 mL) was added triethylamine (0.014 mL, 0.101 mmol, 5.0 equiv.) at room temperature. The reaction was kept at room temperature for 3 hrs and the solvent was concentrated. The product was separated by CombiFlash® and was eluted with 13-16% methanol in dicholoromethane. LC-MS: [M+5H]/5 calculated 974.25, found 975.18.

### Tris(PEG23+CLS)

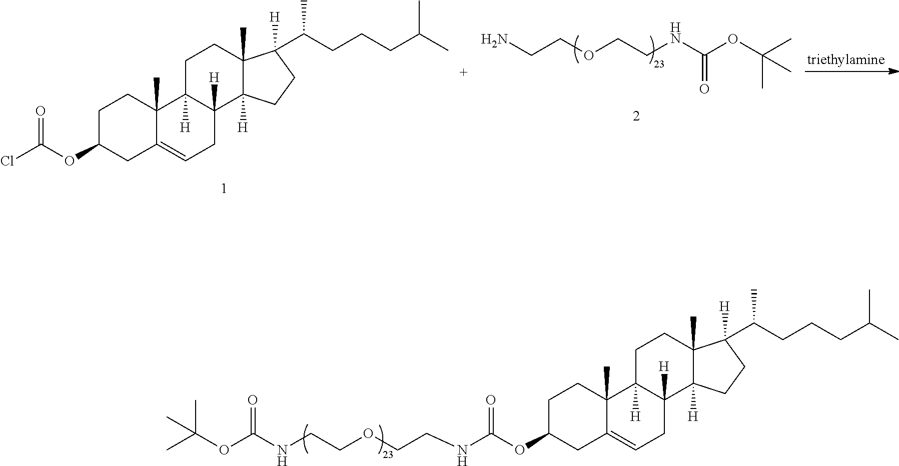

To a solution of compound 1 (100 mg, 0.222 mmol, 1.0 equiv.) and compound 2 (274 mg, 0.233 mmol, 1.05 equiv.) in anhydrous DCM (2 mL) was added triethylamine (0.094 mL, 0.668 mmol, 3.0 equiv.) at room temperature. The reaction was kept at room temperature for 2 hrs and the reaction mixture was concentrated. The product was separated by CombiFlash® and was eluted with 8-15% methanol in dicholoromethane. LC-MS: calculated [M+H₂O]+1603.17, found 1603.18.

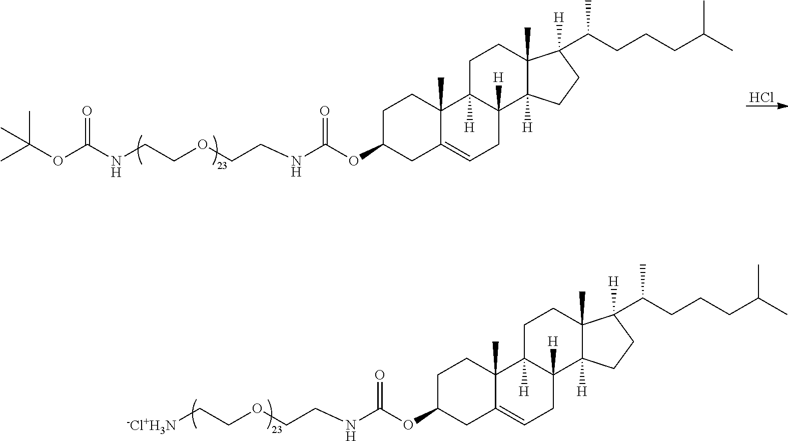

To a solution of compound 1 (353 mg, 0.222 mmol, 1.0 equiv.) in anhydrous 1,4-dioxane (0.5 mL) was added HCl solution in dioxane (1.11 mL, 4.451 mmol, 20 equiv.) at room temperature. The reaction was kept at room temperature for 30 min and the solvent was concentrated. The product was used directly without further purification. LC-MS: calculated [M+H]+ 1486.01, found 1486.50.

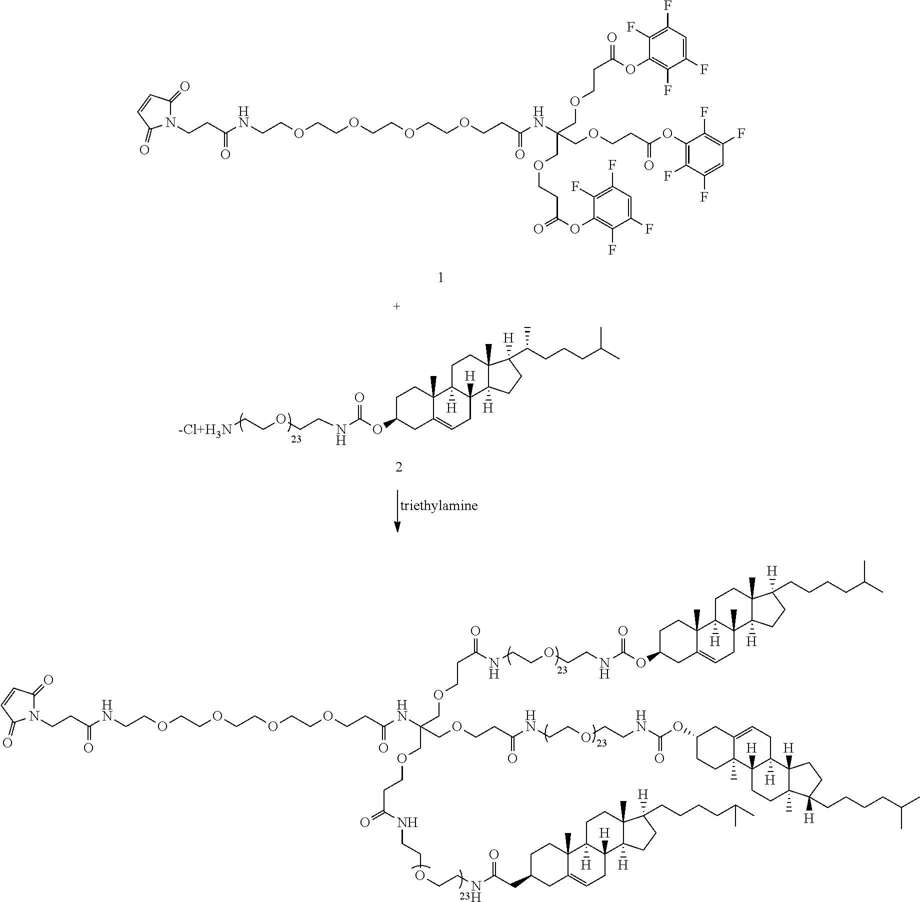

To a solution of compound 1 (24 mg, 0.0203 mmol, 1.0 equiv.) and compound 2 (94 mg, 0.062 mmol, 3.05 equiv.) in anhydrous DMF (2 mL) was added triethylamine (0.014 mL, 0.101 mmol, 5.0 equiv.) at room temperature. The reaction was kept at room temperature for 3 hrs and the solvent was concentrated. The product was separated by CombiFlash® and was eluted with 13-16% methanol in dicholoromethane.

### PEG95+C22

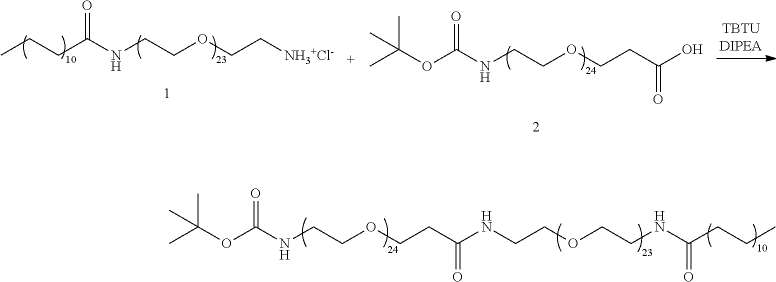

To a solution of compound 1 (60 mg, 0.0419 mmol, 1.0 equiv.), compound 2 (52 mg, 0.0419 mmol, 1.0 equiv.) and diisopropylethylamine (0.022 mL, 0.125 mmol, 3.0 equiv.) in anhydrous DMF (3 mL) was added TBTU (16 mg, 0.0503 mmol, 1.2 equiv.) at room temperature. The reaction was kept at room temperature for 2 hrs. The reaction mixture was concentrated. The product was purified by CombiFlash® and was eluted with 6-8% methanol in dichloromethane. LC-MS: calculated [M+4H]+/4 656.66, found 656.17. Yield: 0.063 g (57.3%.)

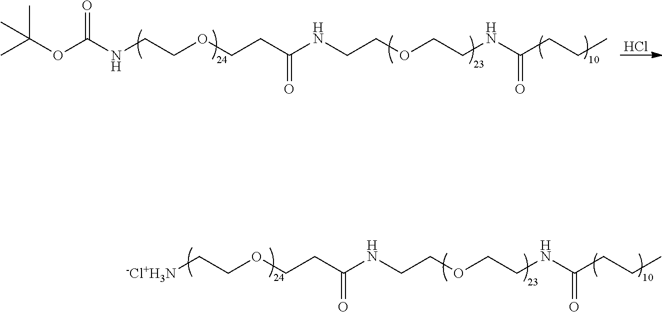

To a solution of compound 1 (60 mg, 0.0229 mmol, 1.0 equiv.) in anhydrous 1,4-dioxane (0.5 mL) was added HCl solution in dioxane (0.286 mL, 1.143 mmol, 50 equiv.) at room temperature. The reaction was kept at room temperature for 30 min and the solvent was concentrated. The product was used directly without further purification. LC-MS: calculated [M+3H]+/3 841.88, found 841.48, calculated [M+4H]+/4 631.66, found 632.41.

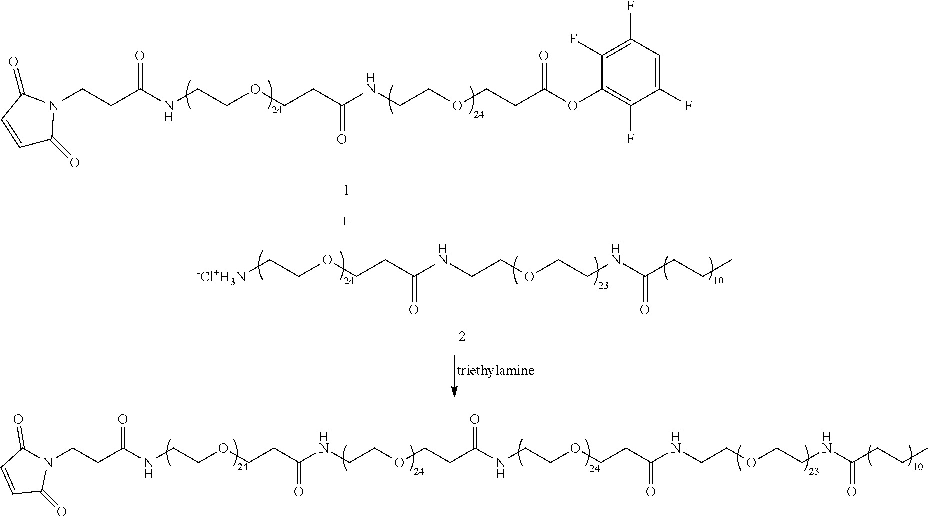

To a solution of compound 1 (55 mg, 0.0214 mmol, 1.0 equiv.) and compound 2 (54.7 mg, 0.0214 mmol, 1.0 equiv.) in anhydrous DMF (2 mL) was added triethylamine (0.009 mL, 0.0641 mmol, 3.0 equiv.) at room temperature. The reaction was kept at room temperature for 2 hrs and the solvent was concentrated. The product was separated by CombiFlash and was eluted with 15-20% methanol in dicholoromethane. LC-MS: calculated [M+5H]+/5 986.80, found 987.19, calculated [M+6H]+/6 822.50, found 822.64.

### PEG47+C22

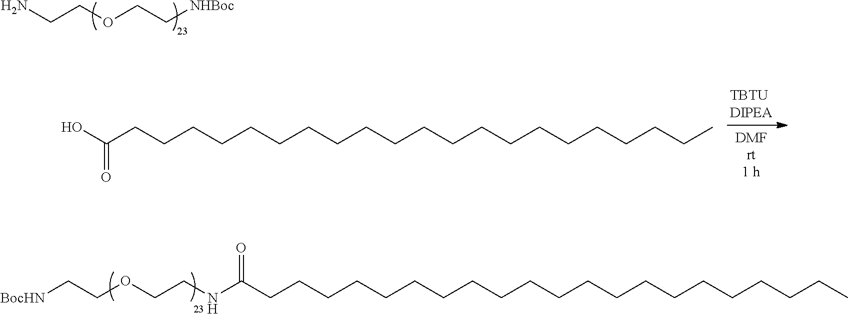

To a solution of compounds 1 (0.200 g) and 2 (0.0580 g) in DMF was added TBTU (0.0657 g) and then DIPEA (0.089 mL) under ambient conditions. The reaction was stirred for 2 h until full conversion was observed by LC-MS. Reaction mixture was immediately concentrated for isolation. The residue was purified by CombiFlash® using silica gel as the stationary phase with a gradient of DCM to 20% MeOH in DCM (0-50%), in which product eluted at 83% B. The product was concentrated under vacuum to provide a clear colorless oil. Yield: 0.161 g (63.0%.) LC-MS: calculated [M+H]+ 1495.05 m/z, observed 1494.30 m/z.

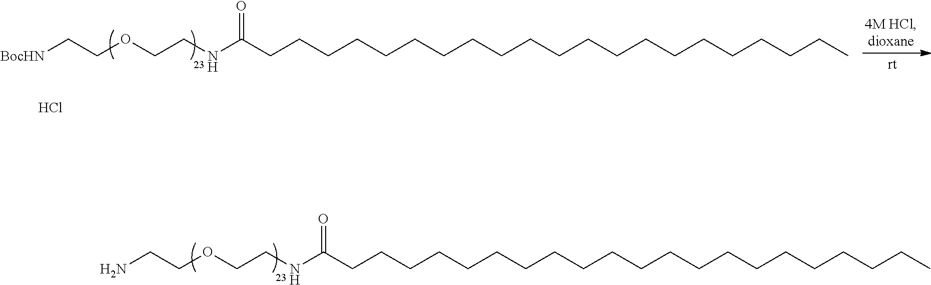

To compound 1 (0.161 g) was added 4 M HCl in dioxane (0.805 mL) at room temperature. The reaction was stirred under ambient conditions. After 10 min., full conversion was confirmed via LC-MS. The reaction mixture was concentrated under vacuum. No isolation was necessary. Concentration provided a white solid. Yield: 0.156 g (101%) LC-MS: calculated [M+H]+ 1396.00 m/z, observed 1396.48 m/z.

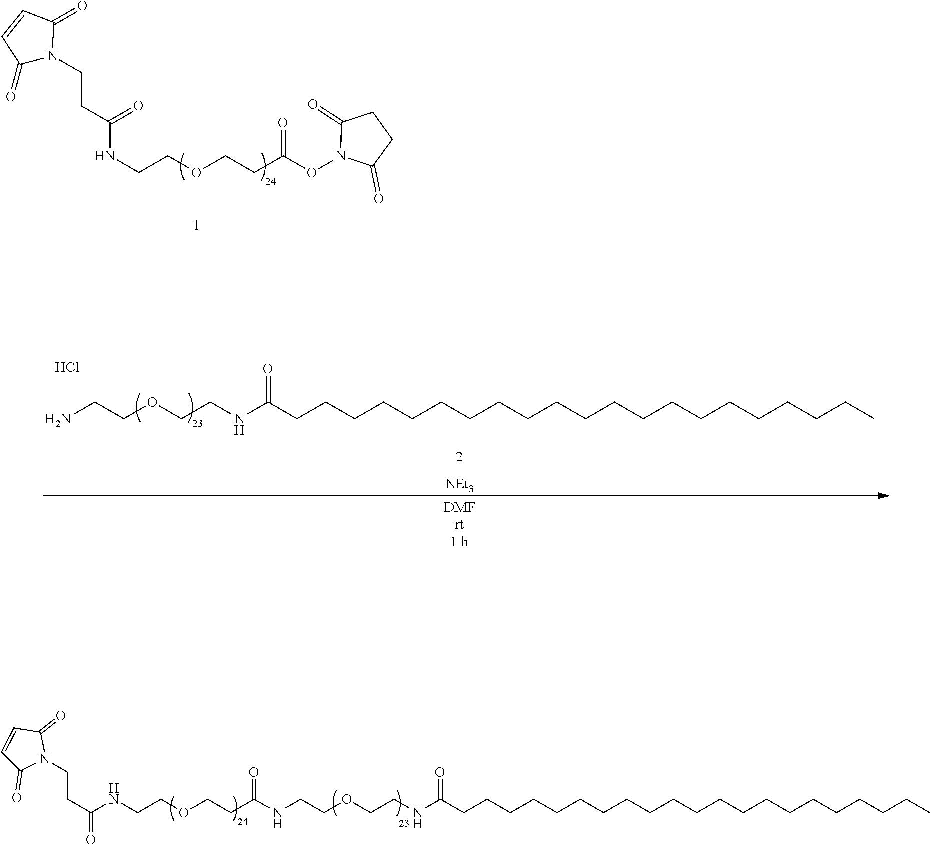

To a solution of compounds 1 (0.152 g) and 2 (0.156 g) in DMF was added NEt₃ (0.046 mL) at room temperature. The reaction was stirred at room temperature. After 1.5 h, full conversion was confirmed by LC-MS. The reaction mixture was concentrated for isolation. The residue was purified by CombiFlash® using silica gel as the stationary phase and was eluted with a gradient of DCM to 20% MeOH in DCM (0-100% B). Product eluted at 100% B. Concentration of fractions provided a white solid. Yield: 0.216 g (74.0%) LC-MS: calculated [M+H]+ 2674.69 m/z, 687.67 m/z (water adduct); observed 686.89 m/z.

### PEG47+CLS

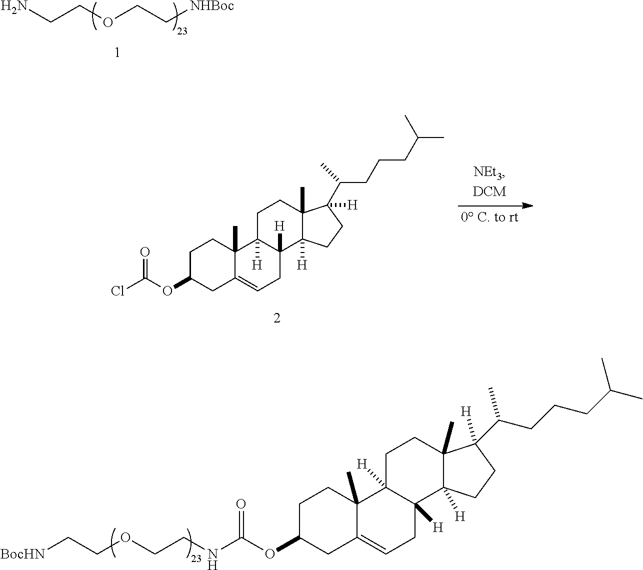

To a solution of compounds 1 (0.200 g) and 2 (0.0765 g) in DCM at 0° C. in ice-water bath was added NEt₃ under normal atmosphere. The reaction was stirred for 10 min. in ice-water bath and then at room temperature for 2 h until full conversion was observed by LC-MS. Reaction mixture was concentrated for isolation. The residue was purified by CombiFlash® using silica gel as the stationary phase with a gradient of DCM to 20% MeOH/DCM (0-50%), in which product eluted at 23% B. The product was concentrated under vacuum to provide a white solid. Yield: 0.156 g (57.8%.) LC-MS: calculated [M+H]+ 1586.06 m/z, observed 1604.14 m/z.

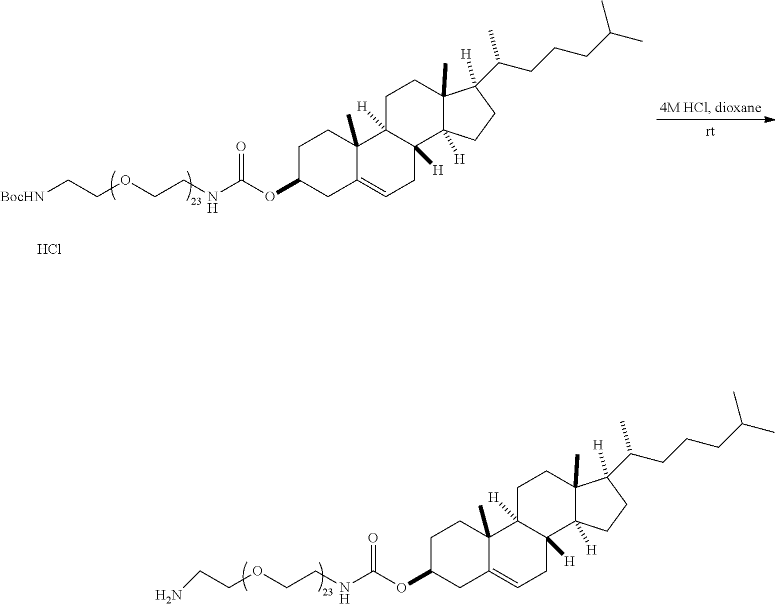

To compound 1 (0.161 g) was added 4 M HCl in dioxane (0.759 mL) at room temperature. The reaction was stirred under ambient conditions. After 10 min., full conversion was confirmed via LC-MS. The reaction mixture was concentrated under vacuum. No isolation was necessary. Concentration provided a white solid. Yield: 0.157 g (102%) LC-MS: calculated [M+H]+ 1486.01 m/z, observed 1487.58 m/z.

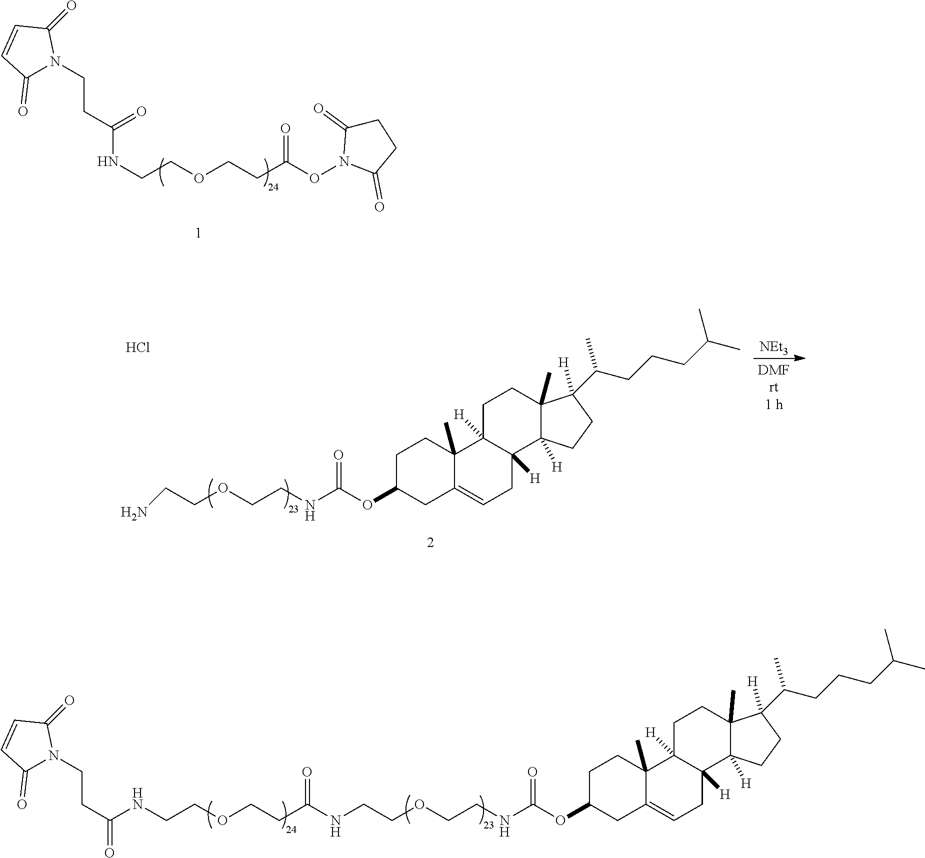

To a solution of compounds 1 (0.144 g) and 2 (0.157 g) in DMF was added NEt₃ (0.043 mL) at room temperature. The reaction was stirred at room temperature. After 1.5 h, full conversion was confirmed by LC-MS. The reaction mixture was concentrated for isolation. The residue was purified by CombiFlash® using silica gel as the stationary phase and was eluted with a gradient of DCM to 20% MeOH in DCM (0-100% B). Product eluted at 37% B. Concentration of fractions provided a white solid. Yield: 0.225 g (79.0%.) LC-MS: calculated [M+H]+ 2764.70 m/z, 710.17 m/z (water adduct); observed 709.46 m/z.

### PEG71+C22

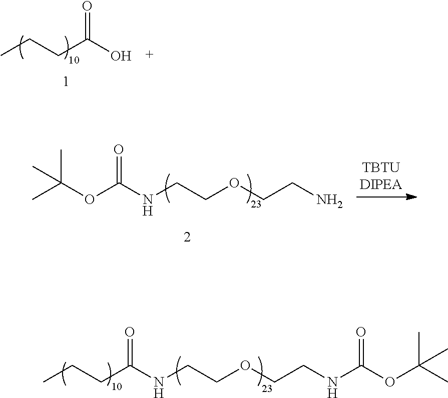

To a solution of compound 1 (50 mg, 0.146 mmol, 1.0 equiv.), compound 2 (172 mg, 0.146 mmol, 1.0 equiv.) and diisopropylethylamine (0.077 mL, 0.440 mmol, 3.0 equiv.) in anhydrous DMF (3 mL) was added TBTU (56 mg, 0.176 mmol, 1.2 equiv.) at room temperature. The reaction was kept at room temperature for 2 hrs. The reaction was quenched with saturated NaHCO₃ aqueous solution (10 mL) and the aqueous was extracted with dichloromethane (3×10 mL). The organic phase was combined, dried over anhydrous Na₂SO₄, and concentrated. The product was purified by CombiFlash® and was eluted with 6-8% methanol in dichloromethane. LC-MS: calculated [M+H]+ 1496.05, found 1496.23.

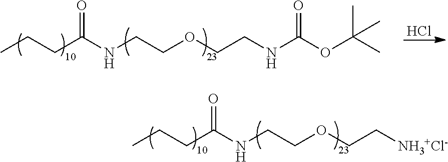

To a solution of compound 1 (120 mg, 0.0802 mmol, 1.0 equiv.) in anhydrous 1,4-dioxane (0.5 mL) was added HCl solution in dioxane (1.00 mL, 4.010 mmol, 50 equiv.) at room temperature. The reaction was kept at room temperature for 30 min and the solvent was concentrated. The product was used directly without further purification. LC-MS: calculated [M+H]+ 1396.00, found 1396.60.

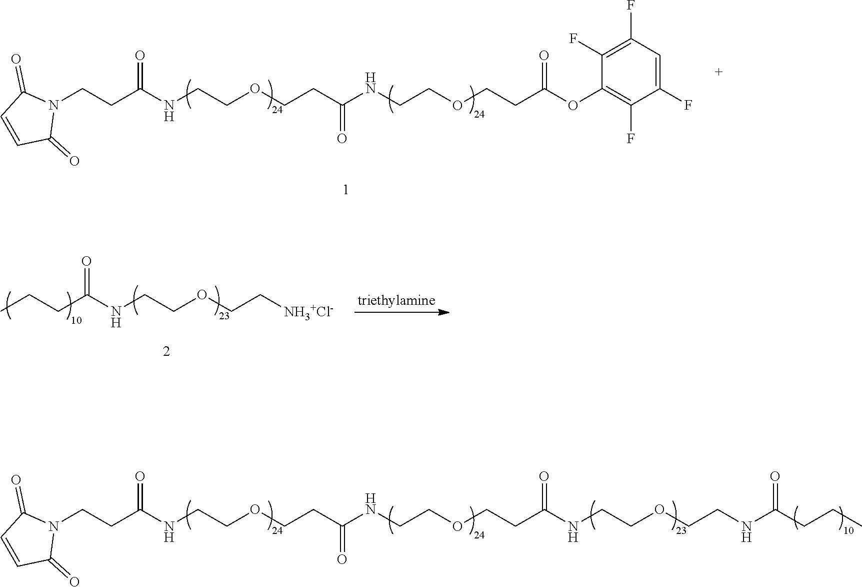

To a solution of compound 1 (98 mg, 0.0381 mmol, 1.0 equiv.) and compound 2 (54.5 mg, 0.0381 mmol, 1.0 equiv.) in anhydrous DMF (5 mL) was added triethylamine (0.016 mL, 0.114 mmol, 3.0 equiv.) at room temperature. The reaction was kept at room temperature for 3 hrs and the solvent was concentrated. The product was separated by CombiFlash and was eluted with 15-20% methanol in dicholoromethane. LC-MS: calculated [M+4H]+/4 951.25, found 952.14, calculated [M+5H]+/5 761.20, found 761.67.

### PEG71+CLS

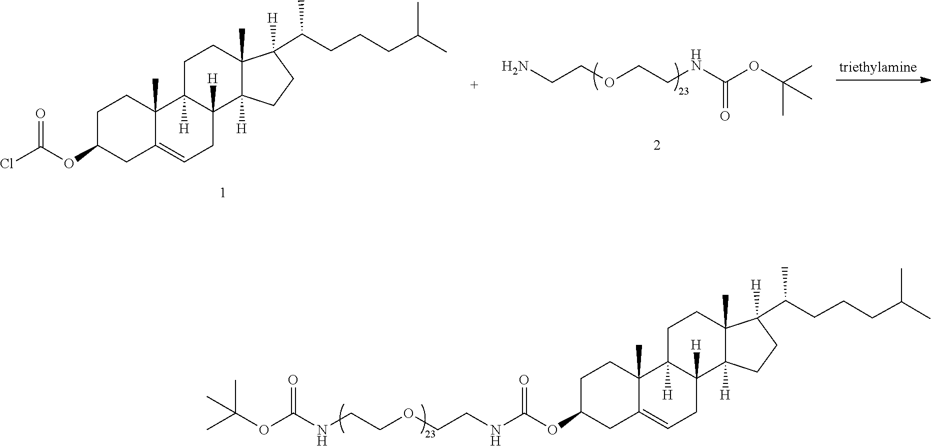

To a solution of compound 1 (100 mg, 0.222 mmol, 1.0 equiv.) and compound 2 (274 mg, 0.233 mmol, 1.05 equiv.) in anhydrous DCM (2 mL) was added triethylamine (0.094 mL, 0.668 mmol, 3.0 equiv.) at room temperature. The reaction was kept at room temperature for 2 hrs and the reaction mixture was concentrated. The product was separated by CombiFlash and was eluted with 8-15% methanol in dicholoromethane. LC-MS: calculated [M+H₂O]+1603.17, found 1603.18.

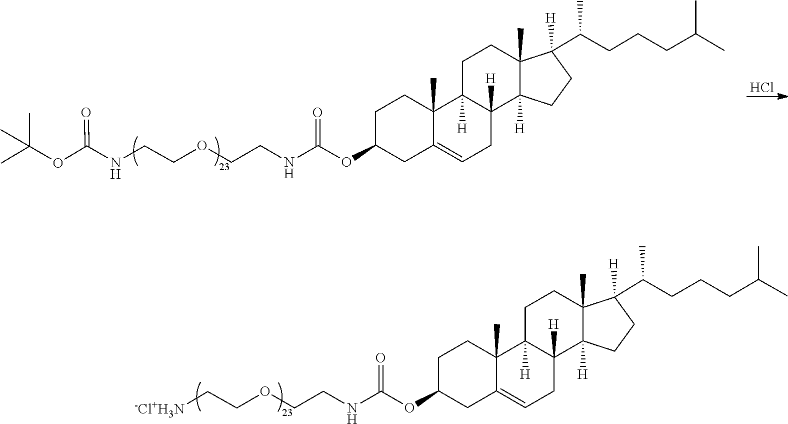

To a solution of compound 1 (353 mg, 0.222 mmol, 1.0 equiv.) in anhydrous 1,4-dioxane (0.5 mL) was added HCl solution in dioxane (1.11 mL, 4.451 mmol, 20 equiv.) at room temperature. The reaction was kept at room temperature for 30 min and the solvent was concentrated. The product was used directly without further purification. LC-MS: calculated [M+H]+ 1486.01, found 1486.50.

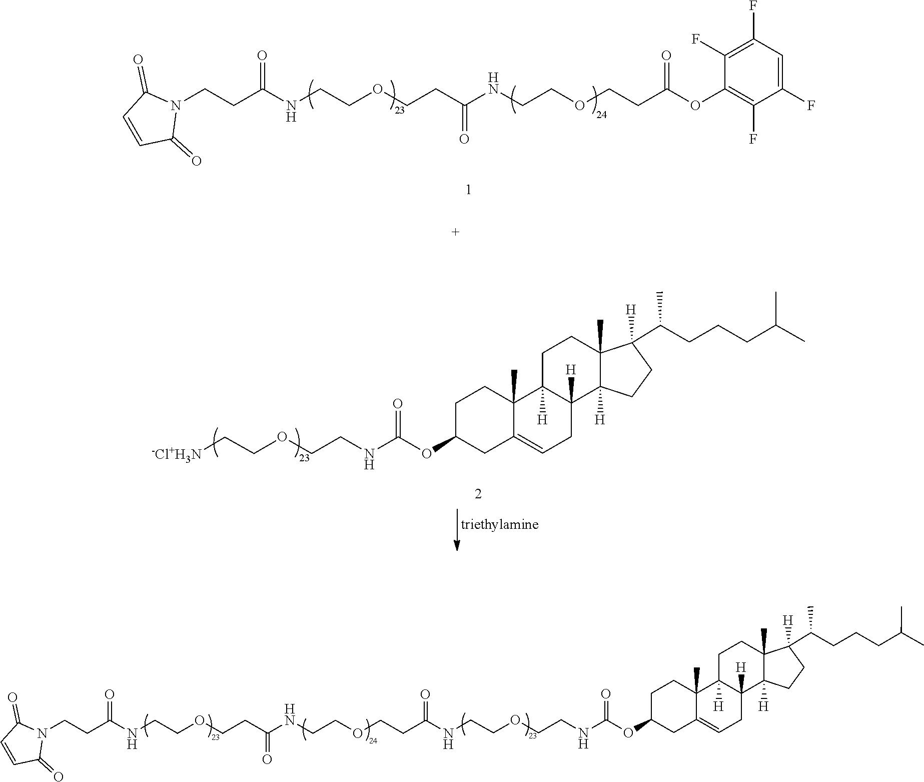

To a solution of compound 1 (70 mg, 0.0272 mmol, 1.0 equiv.) and compound 2 (41.4 mg, 0.0272 mmol, 1.0 equiv.) in anhydrous DMF (2 mL) was added triethylamine (0.012 mL, 0.0816 mmol, 3.0 equiv.) at room temperature. The reaction was kept at room temperature for 3 hrs and the solvent was concentrated. The product was separated by CombiFlash® and was eluted with 13-19% methanol in dicholoromethane. LC-MS: calculated [M+4H]+/4 973.84, found 974.58, [M+5H]+/5 779.27, found 779.79.

### PEG95+CLS

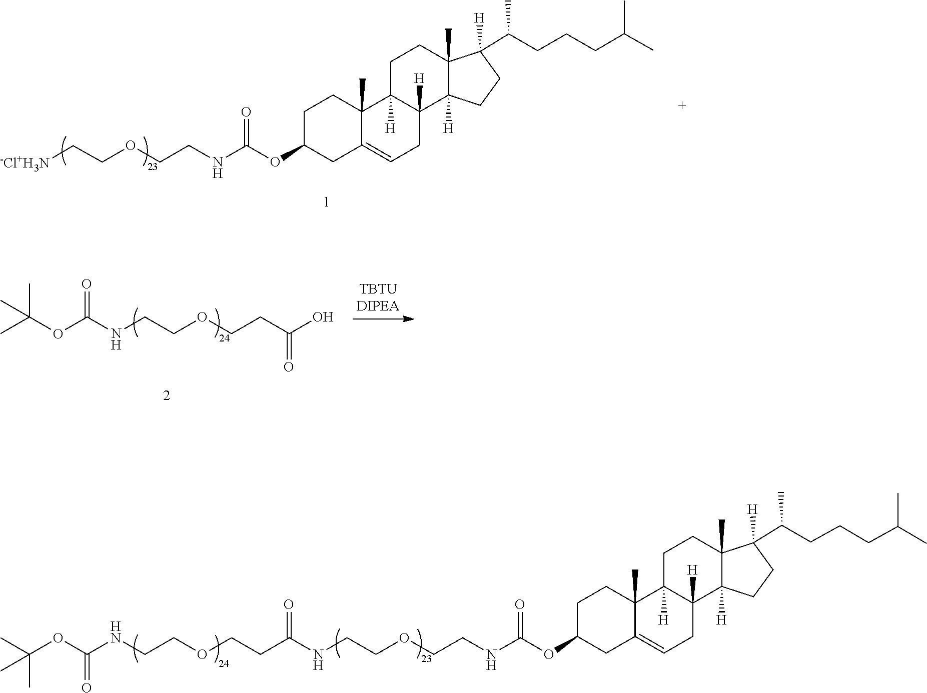

To a solution of compound 1 (60 mg, 0.0419 mmol, 1.0 equiv.), compound 2 (52 mg, 0.0419 mmol, 1.0 equiv.) and diisopropylethylamine (0.022 mL, 0.125 mmol, 3.0 equiv.) in anhydrous DMF (3 mL) was added TBTU (16 mg, 0.0503 mmol, 1.2 equiv.) at room temperature. The reaction was kept at room temperature for 2 hrs. The reaction mixture was concentrated. The product was purified by CombiFlash® and was eluted with 12-18% methanol in dichloromethane. LC-MS: calculated [M+4H]+/4 679.18, found 679.93, [M+3H]+/3 905.24, found 905.81. Yield: 0.082 g (76.7%.)

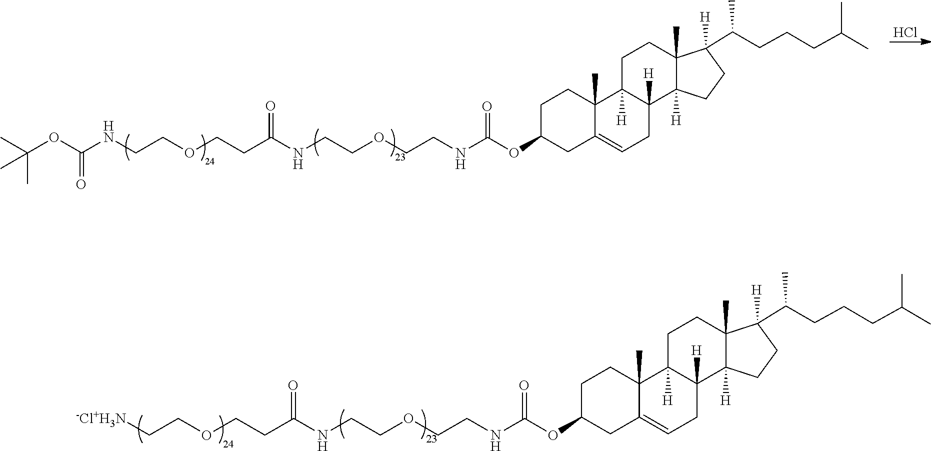

To a solution of compound 1 (85 mg, 0.0313 mmol, 1.0 equiv.) in anhydrous 1,4-dioxane (0.3 mL) was added HCl solution in dioxane (0.391 mL, 1.565 mmol, 50 equiv.) at room temperature. The reaction was kept at room temperature for 30 min and the solvent was concentrated. The product was used directly without further purification. LC-MS: calculated [M+3H]+/3 871.89, found 871.72, [M+4H]+/4 654.17, found 654.97.

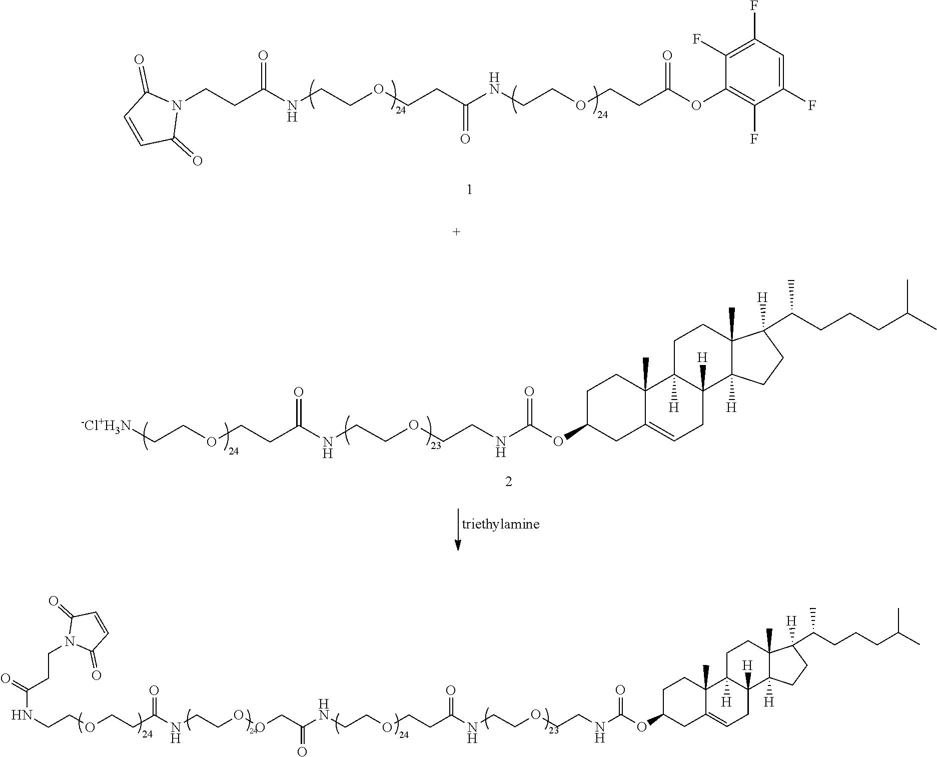

To a solution of compound 1 (80 mg, 0.0311 mmol, 1.0 equiv.) and compound 2 (82 mg, 0.0311 mmol, 1.0 equiv.) in anhydrous DMF (2 mL) was added triethylamine (0.013 mL, 0.0932 mmol, 3.0 equiv.) at room temperature. The reaction was kept at room temperature for 3 hrs and the solvent was concentrated. The product was separated by CombiFlash® and was eluted with 13-19% methanol in dicholoromethane. LC-MS: calculated [M+4H]+/4 1255.76, found 1255.57, [M+5H]+/5 1004.81, found 1005.79.

### Synthesis of LP1-p

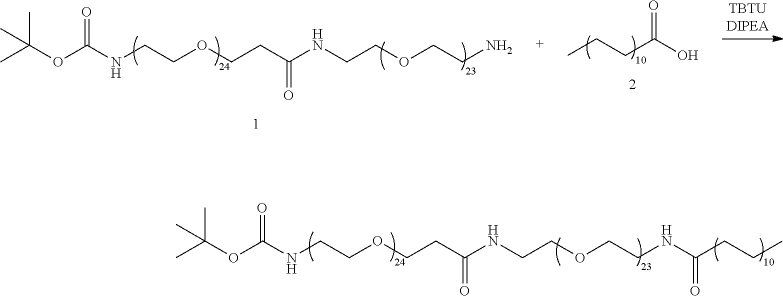

To a solution of compound 1 (2630 mg, 1.142 mmol, 1.0 equiv.), compound 2 (428 mg, 1.256 mmol, 1.1 equiv.) and diisopropylethylamine (0.597 mL, 3.427 mmol, 3.0 equiv.) in anhydrous DMF (10 mL) was added TBTU (440 mg, 1.371 mmol, 1.2 equiv.) at room temperature. The reaction was kept at room temperature for 2 hrs. The reaction mixture was concentrated. The product was purified by CombiFlash and was eluted with 12-17% methanol in dichloromethane. LC-MS: calculated [M+4H]+/4 656.66, found 656.65.

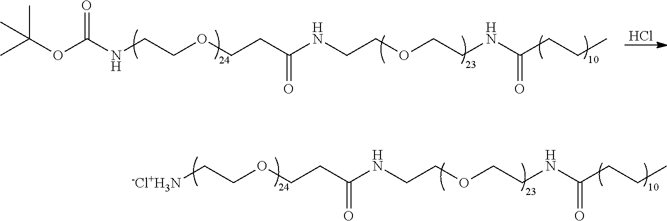

To solid of compound 1 (1150 mg, 0.438 mmol, 1.0 equiv.) was added HCl solution in dioxane (5.478 mL, 21.910 mmol, 50 equiv.) at room temperature. The reaction was kept at room temperature for 30 min and the product was concentrated. The product was used directly without further purification. LC-MS: calculated [M+3H]+/3 841.88, found 842.56, calculated [M+4H]+/4 631.66, found 632.41.

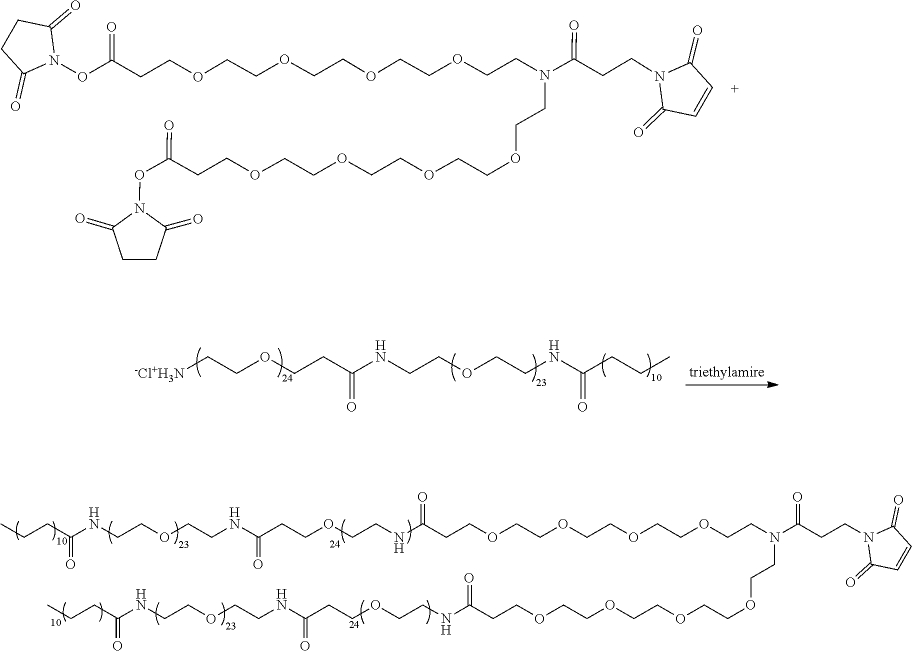

To a solution of compound 1 (175 mg, 0.203 mmol, 1.0 equiv.) and compound 2 (1095 mg, 0.427 mmol, 2.1 equiv.) in anhydrous DCM (10 mL) was added triethylamine (0.144 mL, 1.018 mmol, 5.0 equiv.) at room temperature. The reaction was kept at room temperature for 3 hrs and the solvent was concentrated. The product was separated by CombiFlash and was eluted with 10-17% methanol in dicholoromethane. LC-MS: calculated [M+6H]+/6 946.60, found 947.10, calculated [M+7H]+/7 811.51, found 811.35.

### Synthesis of LP5-p

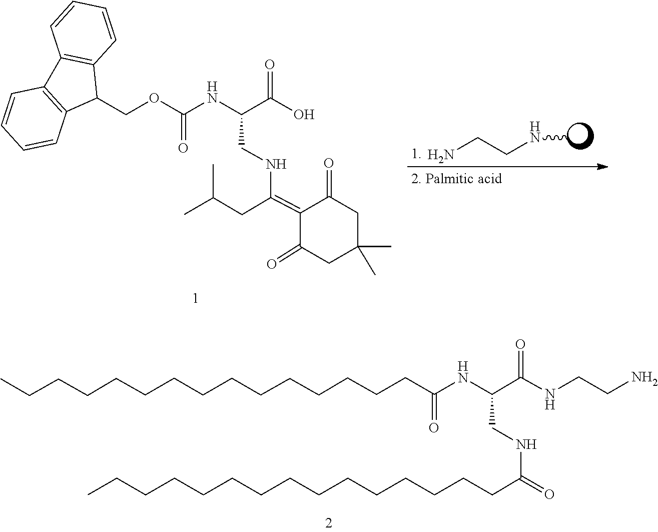

Compound 1 (105 mg, 0.198 mmol) in DMF was treated with TBTU (4 eq) and agitated for 5 minutes. DIEA (8eq) was subsequently added and the mixture was added to 1 molar eq. of ethylamine diamine on pre-swelled 2-chlorotrityl resin. After agitation for 30 minutes the resin was washed three times with DMF and then treated with 2% hydrazine in DMF for 10 minutes. Coupling of palmitic acid (202 mg, 0.789 mmol) was repeated using the same procedure as the coupling of compound 1. Upon completion the resin was washed with 3 portions of DCM and treated with a 1% solution of TFA in DCM for 10 minutes. TFA treatment was repeated and the resin was washed with 3 portions of DCM. All volatiles were removed and the crude was used without further purificaiton. Yield 126 mg (81%).

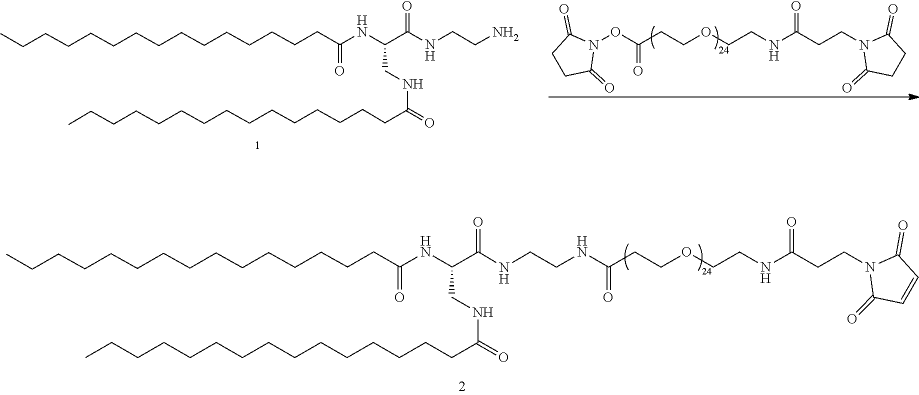

To a mixture containing compound 1 (23 mg, 37 umol) and DIEA (14.1 uL, 81 umol) in DMF (1 mL) was added NHS-PEG24-MAL (61.5 mg, 0.0441 mmol) and the reaction was stirred for 30 minutes. Upon completion the crude dry loaded onto silica and compound 2 was isolated eluting a gradient of MeOH in DCM. Yield 15 mg (21%).

### Synthesis of LP28-p

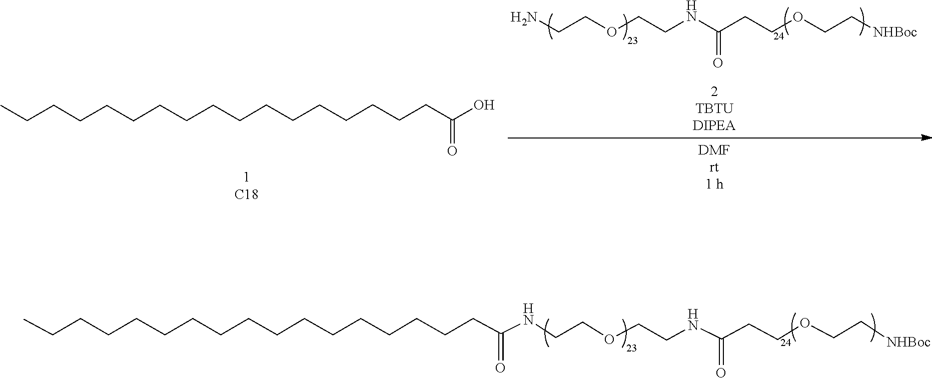

To a solution of compounds 1 (80 mg) and 2 (60.2 mg) in DMF was added TBTU (90.3 mg) and then DIPEA (0.147 mL) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-80%, isocratic, and then to 100%) over 20-30 min., in which product eluted at 68% B. The product was concentrated under vacuum to provide a white oily residue. LC-MS: calculated [M+H]+ 2567.65 m/z, observed 1301.78 (+2/2, +H₂O) m/z.

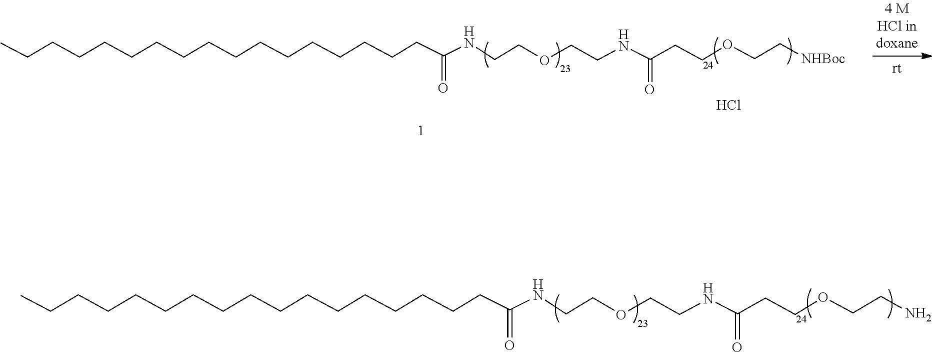

To compound 1 (100.4 mg) was added 4 M HCl/dioxane (14.3 mg) at room temperature. The reaction was stirred under ambient conditions. The reaction was stirred overnight until full conversion was confirmed via LC-MS. The reaction mixture was azeotroped with PhMe and concentrated under vacuum overnight to provide an oil. LC-MS: calculated [M+H]+ 2467.60 m/z, observed 1243.32 m/z.

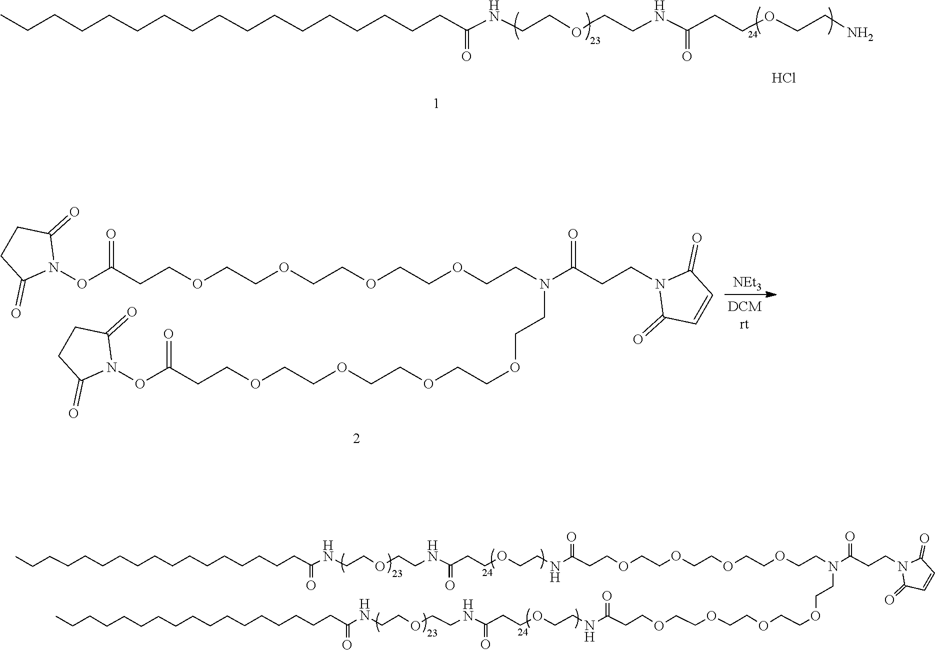

A solution of compound 1(97.9 mg) and NEt₃ (0.016 mL) in anhydrous DCM was prepared and stirred under sparging nitrogen atmosphere. Compound 2 (15.8 mg) was then added to reaction mixture. The reaction mixture was stirred at room temperature until full conversion was observed by LC-MS. The reaction mixture was directly concentrated for isolation. The residue was purified by CombiFlash using silica gel as the stationary phase and was eluted with a gradient of DCM to 20% MeOH in DCM (0-100% B). Product eluted at 67% B. LC-MS: calculated [M+H]+ 5562.48 m/z, observed 1409.68 (+4/4, +H₂O) m/z.

### Synthesis of LP29-p

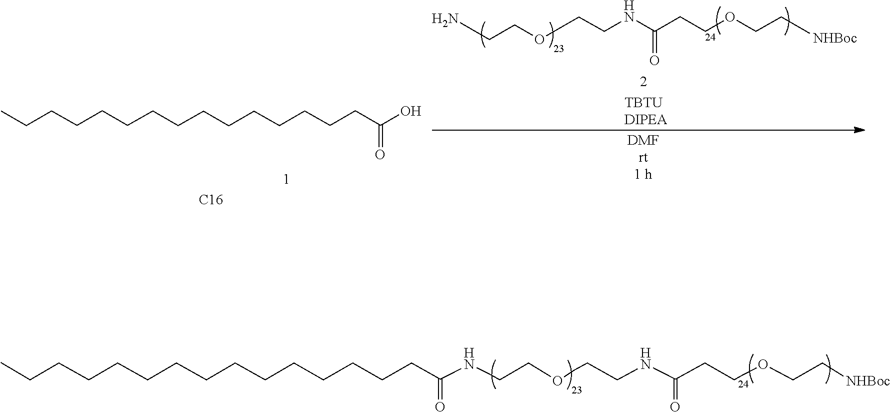

To a solution of compounds 1 (40 mg) and 2 (334 mg) in DMF was added TBTU (50.1 mg) and then DIPEA (0.082 mL) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-80%) over 20-30 min., in which product eluted at 71% B. The product was concentrated under vacuum to provide a white oily residue. LC-MS: calculated [M+H]+ 2539.62 m/z, observed 1288.21 (+2/2, +H₂O) m/z.

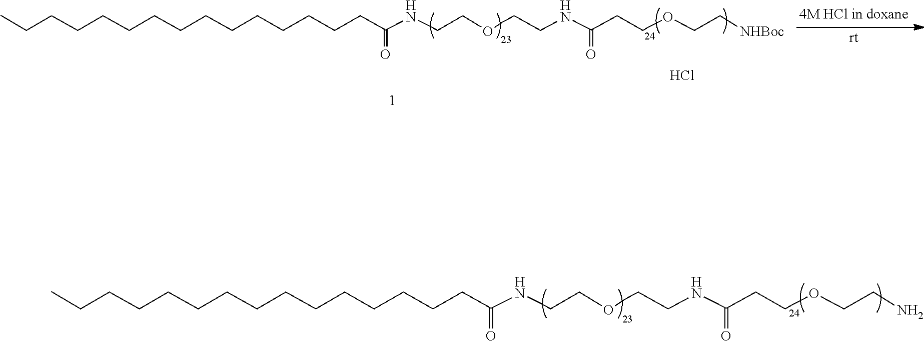

To compound 1 (147 mg) was added 4 M HCl/dioxane (21.2 mg) at room temperature. The reaction was stirred under ambient conditions. The reaction was stirred overnight until full conversion was confirmed via LC-MS. The reaction mixture was azeotroped with PhMe and concentrated under vacuum overnight to provide an oil. LC-MS: calculated [M+H]+ 2439.57 m/z, observed 611.16 (+4/4) m/z.

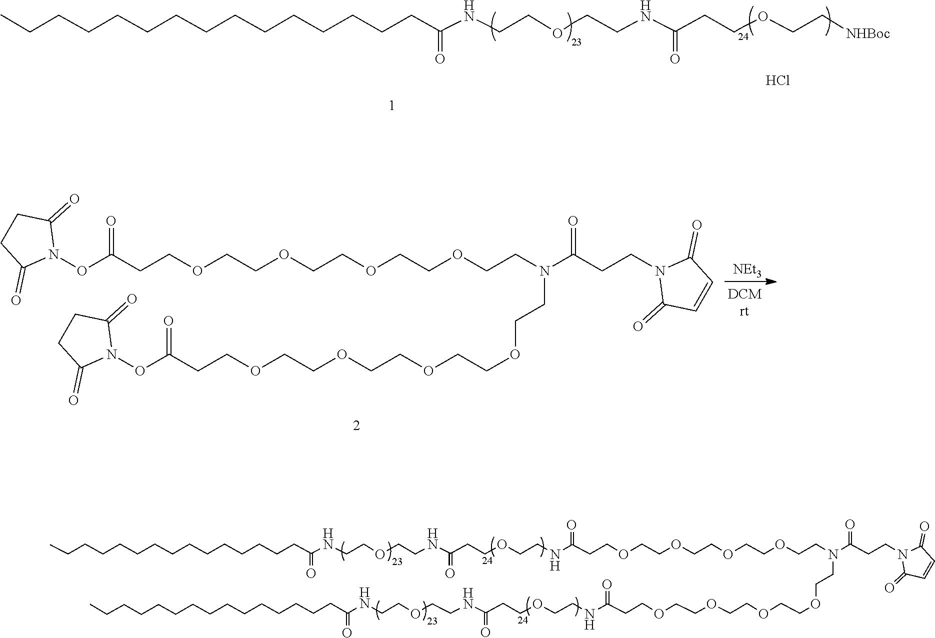

A solution of compound 1 (143 mg) and NEt₃ (0.024 mL) in anh. DCM was prepared and stirred under sparging nitrogen atmosphere. Compound 2 (23.4 mg) was then added to the reaction mixture. The reaction mixture was stirred at room temperature until full conversion was observed by LC-MS.

The reaction mixture was directly concentrated for isolation. The residue was purified by CombiFlash using silica gel as the stationary phase and was eluted with a gradient of DCM to 20% MeOH in DCM (0-100% B). Product eluted at 54% B. LC-MS: calculated [M+H]+ 5506.42 m/z, observed 1854.41 (+3/3, +H₂O) m/z.

### Synthesis of LP33-p

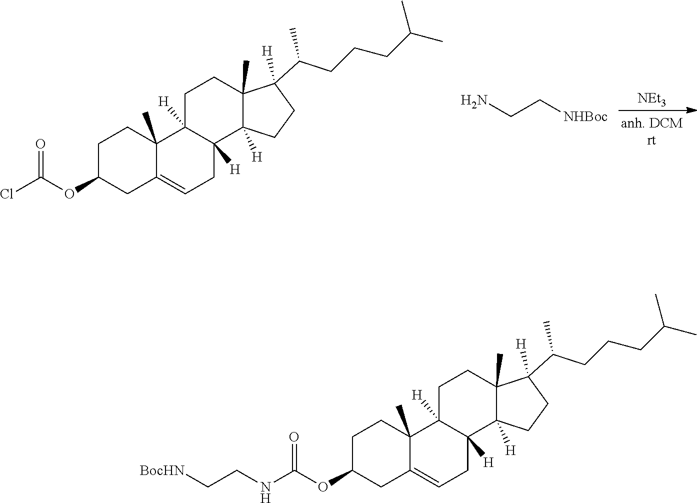 [SMILES: CC(C)CCC[C@@H](C)[C@H]1CC[C@H]2[C@@H]3CC=C4C[C@@H](OC(=O)Cl)CC[C@]4(C)[C@H]3CC[C@]12C ; CC(C)(C)OC(=O)NCCN ; CC(C)CCC[C@@H](C)[C@H]1CC[C@H]2[C@@H]3CC=C4C[C@@H](OC(=O)NCCNC(=O)OC(C)(C)C)CC[C@]4(C)[C@H]3CC[C@]12C ; "NEt3"]

To a solution of compounds 1 (2.00 g, 4.45 mmol) and 2 (1.07 g, 6.68 mmol) in anh. DCM, NEt₃ (1.86 mL, 13.4 mmol) was added under ambient conditions. Reaction was stirred until full conversion was observed by LC-MS. The reaction mixture was then directly concentrated. The residue was purified by CombiFlash using silica gel as the stationary phase with a gradient of DCM to 20% MeOH/DCM (0-100%) over 45 min., in which product eluted at 8% B. Product was concentrated to provide a white solid. LC-MS: calculated [M+H]+ 573.46 m/z, observed 573.60 m/z.

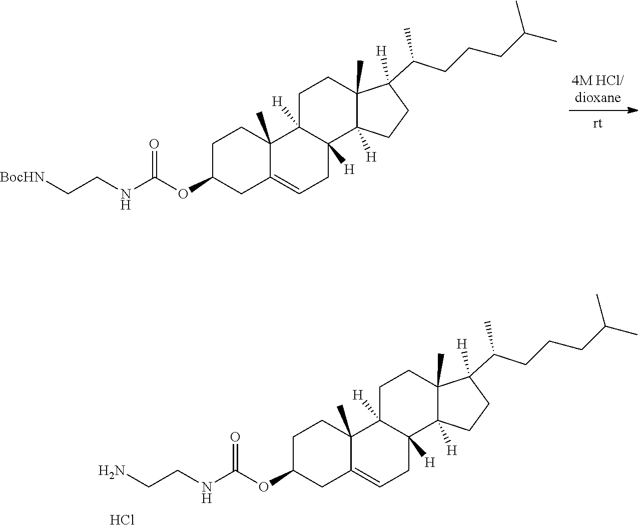 [SMILES: CC(C)CCC[C@@H](C)[C@H]1CC[C@H]2[C@@H]3CC=C4C[C@@H](OC(=O)NCCNC(=O)OC(C)(C)C)CC[C@]4(C)[C@H]3CC[C@]12C ; CC(C)CCC[C@@H](C)[C@H]1CC[C@H]2[C@@H]3CC=C4C[C@@H](OC(=O)NCCN)CC[C@]4(C)[C@H]3CC[C@]12C ; Cl]

To compound 1 (317 mg, 0.553 mmol) was added 4 M HCl/dioxane (1.383 mL) at rt. The reaction was stirred under ambient conditions. Reaction was stirred overnight until full conversion was confirmed via LC-MS. The reaction mixture was concentrated under high-vacuum overnight to provide a clear and colorless greasy residue. LC-MS: calculated [M+H]+ 473.40 m/z, observed 473.58 m/z.

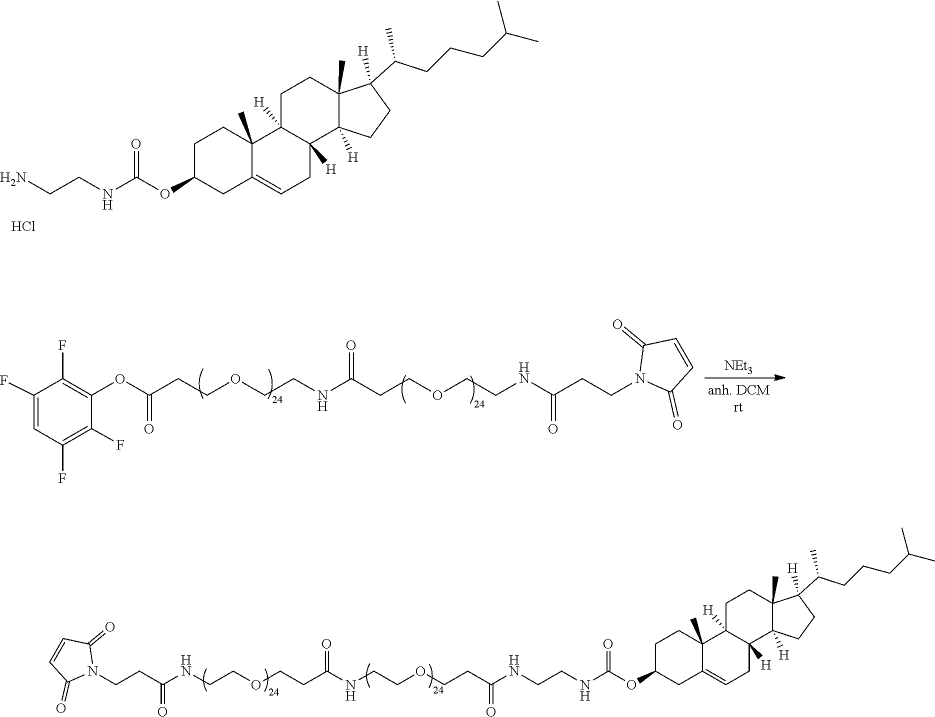

To a solution of compounds 1 (282 mg, 0.553 mmol) and 2 (1.35 g, 0.526 mmol) in anh. DCM under N₂(g), NEt₃ (0.386 mL) was added. Reaction was stirred until full conversion was observed by LC-MS. The reaction mixture was then directly concentrated. The residue was purified by CombiFlash using silica gel as the stationary phase with a gradient of DCM to 20% MeOH/DCM (0-100%) over 45 min., in which product eluted at 46% B. Product was concentrated to provide a white solid. LC-MS: calculated [M+H]+ 2879.76 m/z, observed 960.98 (+3/3) m/z.

### Synthesis of LP38-p

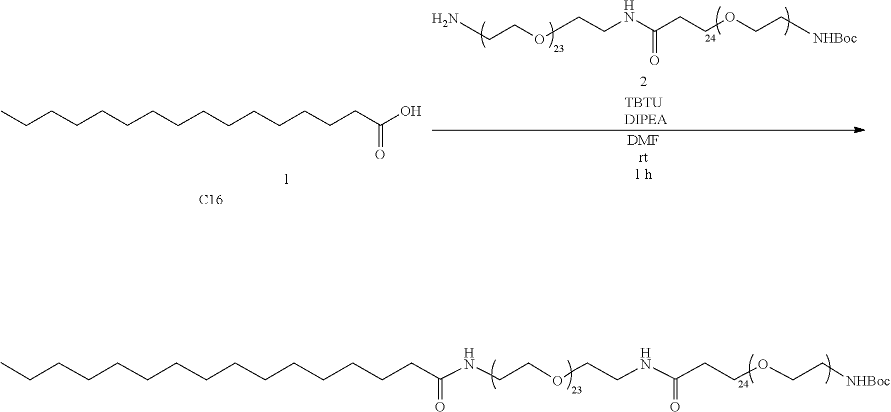

To a solution of compounds 1 (35 mg) and 2 (299 mg) in DMF was added TBTU (43.8 mg) and then DIPEA (0.071 mL) under ambient conditions. Reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-100%) over 20-30 min., in which product eluted at 56% B. The product was concentrated under vacuum to provide a white oily residue. LC-MS: calculated [M+H]+ 2539.62 m/z, observed 1288.07 (+2/2, +H₂O) m/z.

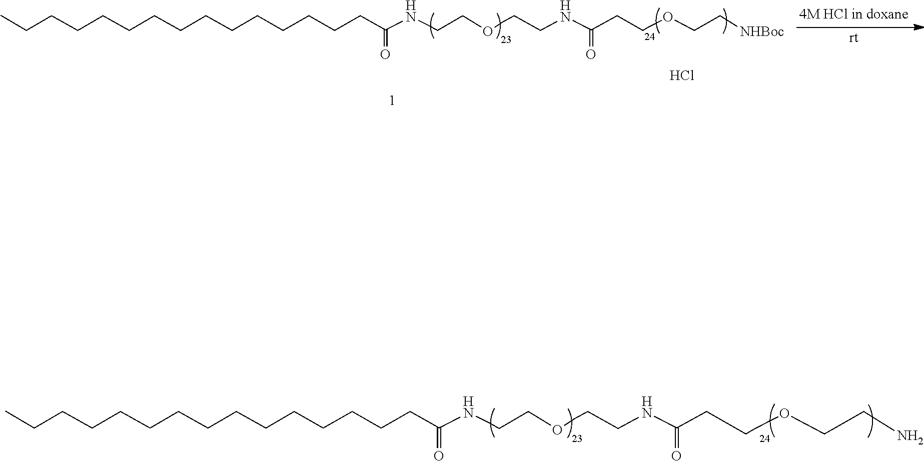

To compound 1 (186 mg) was added 4 M HCl/dioxane (26.7 mg) at room temperature. The reaction was stirred under ambient conditions. The reaction was stirred overnight until full conversion was confirmed via LC-MS. The reaction mixture was azeotroped with PhMe and concentrated under vacuum overnight to provide an oil. LC-MS: calculated [M+H]+ 2439.57 m/z, observed 1220.97 (+2/2) m/z.

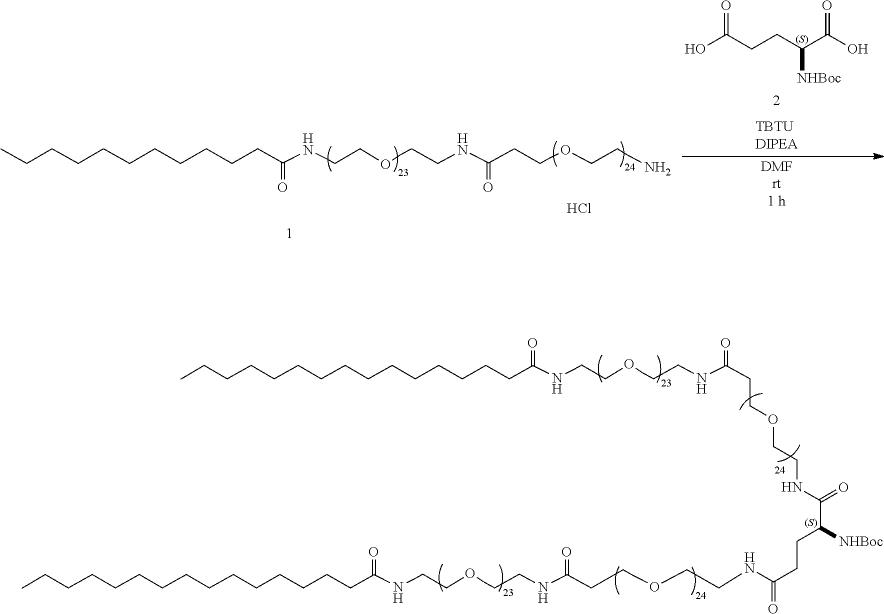

To a solution of compound 1 (181 mg), TBTU (24 mg), and DIEA (0.033 mL) in DMF was added 2 (8.7 mg) under ambient conditions. Reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-100%) over 20-30 min., in which product eluted at 65% B. The product was concentrated under vacuum to provide a white oily residue. LC-MS: calculated [M+H]+ 5089.22 m/z, observed 1036.24 (+5/5, +H₂O) m/z.

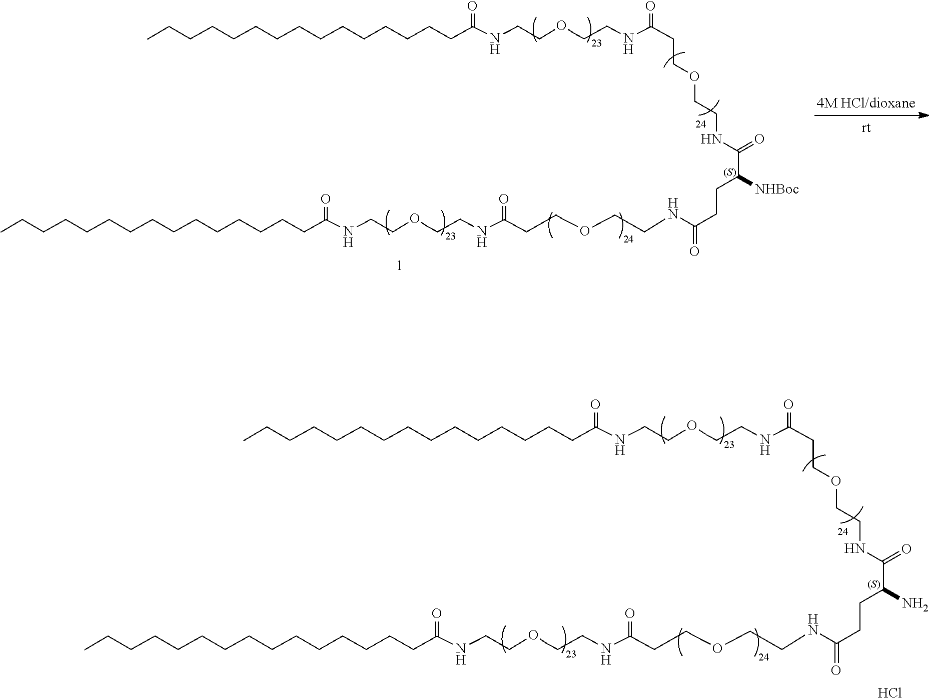

To compound 1 (130 mg) was added 4 M HCl/dioxane (9.3 mg) at rt. The reaction was stirred under ambient conditions. Reaction was stirred overnight until full conversion was confirmed via LC-MS. The reaction mixture was azeotroped with PhMe and concentrated under vacuum overnight to provide an oil. LC-MS: calculated [M+H]+ 4989.17m/z, observed 1248.58 (+4/4) m/z.

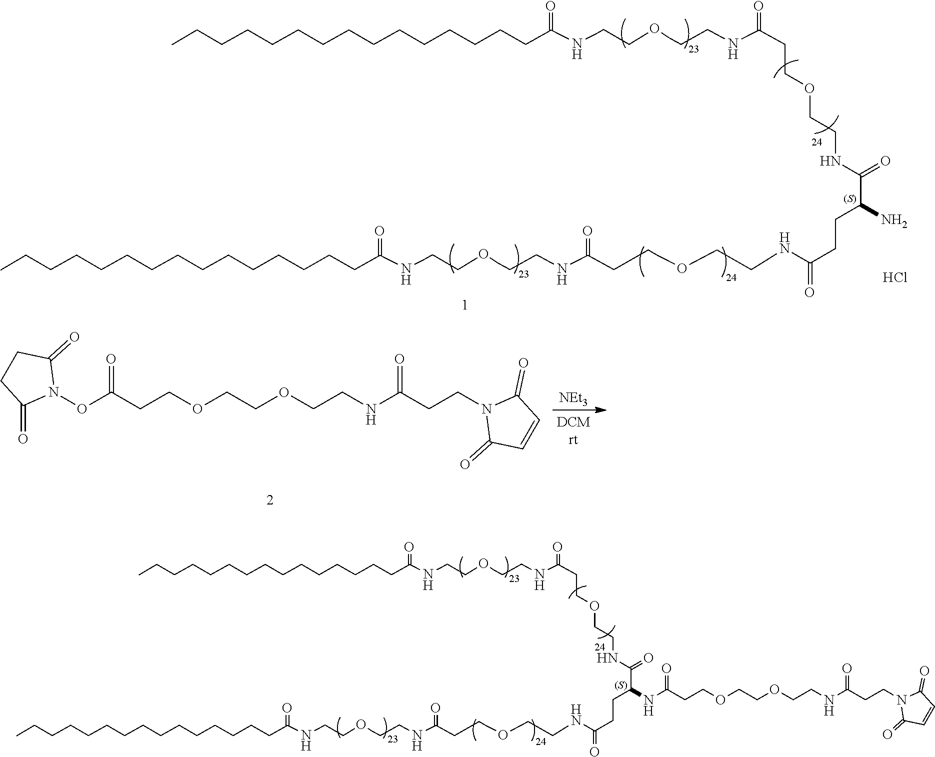

A solution of compound 1 (128 mg) and NEt₃ (0.018 mL) in anhydrous DCM under sparging N₂(g) was prepared at room temperature. Compound 2 (10.3 mg) was then added slowly. The reaction mixture was allowed to stir until full conversion was observed by LC-MS. The reaction mixture was allowed to stir until full conversion was observed by LC-MS. The reaction mixture was then directly concentrated. The residue was purified by CombiFlash using silica gel as the stationary phase with a gradient of DCM to 20% MeOH/DCM (0-100%) over 30 min., in which product eluted at 100% B. Product was concentrated to provide a white solid. LC-MS: calculated [M+H]+ 5299.28 m/z, observed 1786.62 (+3/3, +H₂O) m/z.

### Synthesis of LP39-p

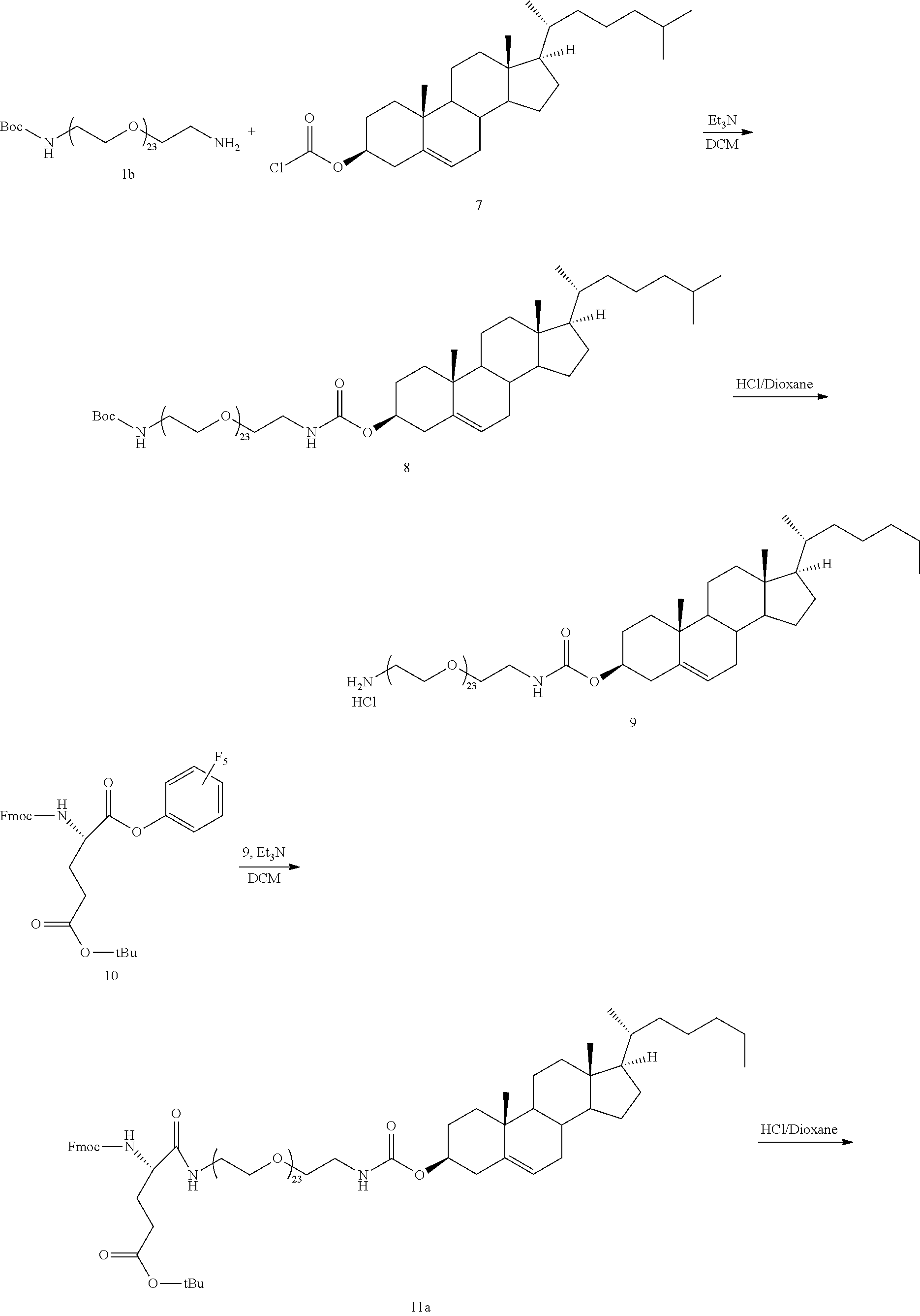

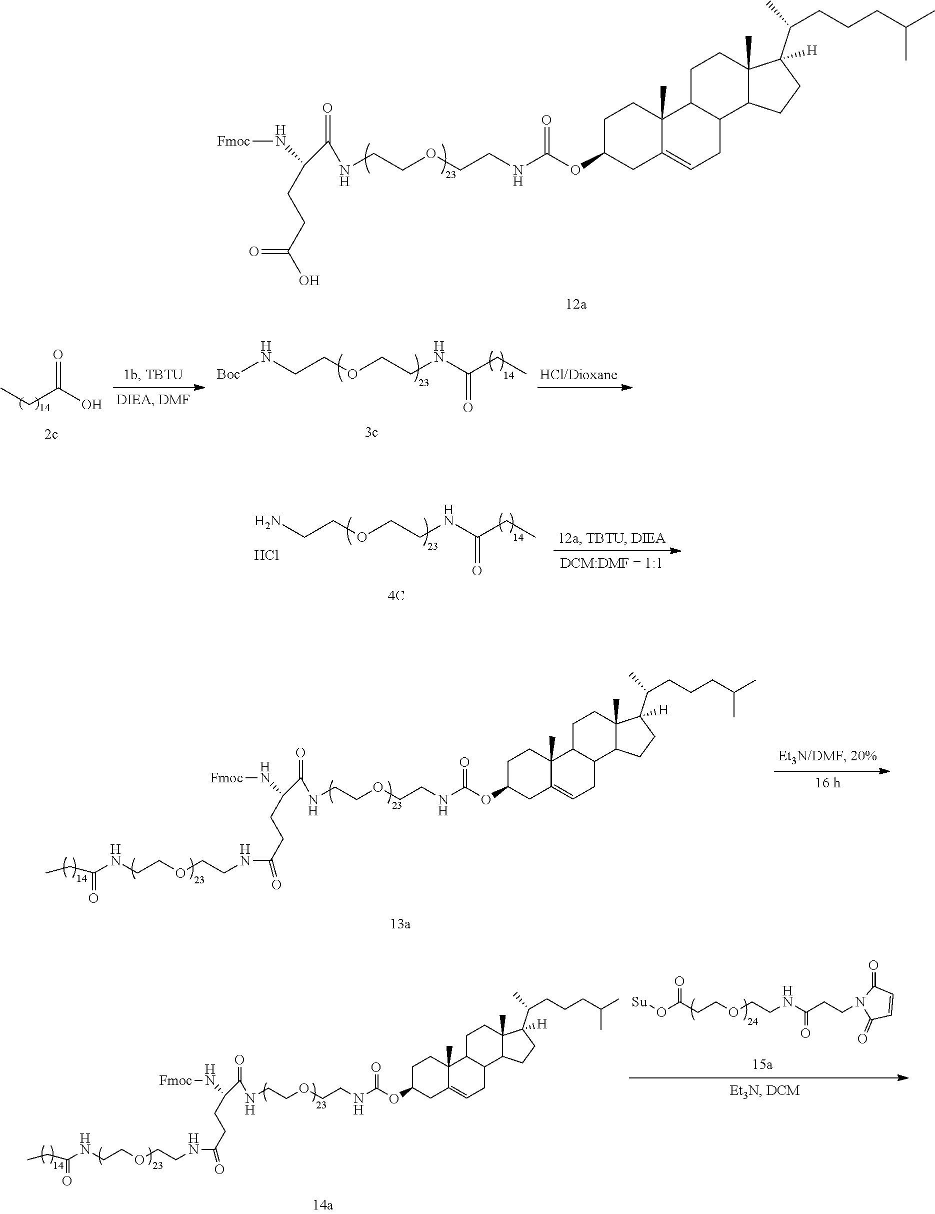

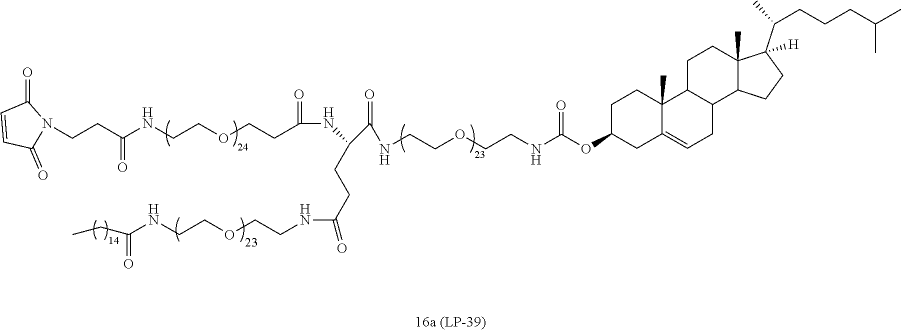

Boc-protected PEG₂₃-amine 1b (Quanta Biodesign Limited, 200 mg, 0.17 mmol) was stirred with cholesterol chloroformate 7 (77 mg, 0.17 mmol) and Et3N (48 uL, 0.341 mmol) in 5 mL of DCM for 1.5 h. The solvent was removed in vacuo, the residue was mixed with SiO₂ (1 g) and loaded on CombiFlash. The product was purified using the system DCM: 20% McOH in DCM, gradient 0-80%, 40 min. Calculated MW 1586.09, M+18=1604.09, (M+2×18)/2=811.05 Found: MS (ES, pos): 1603.55 [M⁺NH₄]⁺, 811.07 [M⁺²NH₄]²⁺.

Product 8 was Boc-deprotecterd as described for 3a and the hydrocloride salt 9 (62 mg, 0.04 mmo) was stirred with pentafluorophenyl ester 10 (24 mg, 0.04 mmol) and Et3N (14 uL, 0.1 mmol) in DCM (5 mL) for 1.5 h. The solvent was removed in vacuo, the residue was mixed with SiO₂ (400 mg) and loaded on CombiFlash. The product 11a was purified using the system DCM: 20% MeOH in DCM, gradient 0-70%, 30 min. Yield 57 mg. Calculated MW 1893.44, M+18=1911.44, (M+2×18)/2=964.72 Found: MS (ES, pos): 1911.00 [M+NH₄]⁺, 964.46 [M+2NH₄]²⁺.

Product 11a was treated with 4M HCl in dioxane (10 mL) for 4 h at RT. The solvent was removed in vacuo, toluene was evaporated 2 times from the residue, product 12a was dried and used directly in the next step.

Solid TBTU (50 mg, 0.156 mmol) was added to a solution of Boc-protected PEG₂₃-amine 1b (Quanta Biodesign Limited, 152 mg, 0.13 mmol), palmitic acid 2c (33 mg, 0.13 mmol), and DIEA (68 uL, 0.39 mmol) in DMF (9 mL). The reaction mixture was sonicated to dissolve solids and stirred for 16 h at RT. The solvent was removed in high vacuo, toluene was evaporated twice from the residue, the residue was dissolved in chloroform (50 mL), washed with NaHCO₃(2×10 mL) and brine (10 mL). Product was dried (Na₂SO₄), concentrated in vacuo, and purified on CombiFlash (SiO₂) using the system DCM: 20% MeOH in DCM, gradient 0-80%, 20 min. Calculated MW 1411.85, M+18=1429.85, (M+1+18)/2=715.43 Found: MS (ES, pos): 1429.24 [M+NH₄]⁺, 715.41 [M+H+NH₄]²⁺.

3c was Boc-deprotected with HCl/dioxane solution and the product was used directly in the next step.

The derivative 12a (60 mg, 0.028 mmol) was stirred with hydrochloride 4c (42 mg, 0.03 mmol), TBTU (11 mg, 0.034 mmol) and DIEA (18 uL, 0.1 mmol) in DCM:DMF=1:1 (8 mL) for 3 h. The solvent was removed in vacuo, toluene was evaporated 2 times from the residue, and the solid was suspended in CHCl₃ (50 mL). The suspension was washed twice with 2% NaHCO₃ and brine. Following concentration in vacuo the product 13a was purified on CombiFlash (DCM: 20% MeOH in DCM, gradient 0-70%, 35 min.)

The product 13a (51 mg, 0.0162 mmol) was stirred with Et3N in DMF (20%, 3 mL) for 16 h, the solvent with Et3N was removed in vacuo, toluene was evaporated 3 times from the residue to obtain deprotected amine 14a. Calculated MW 2908.81, (M+1+18)/2=1463.91, (M+1+18×2)/3=981.94 Found: MS (ES, pos): 1463.69 [M+H+NH₄]²⁺, 981.99 [M+H+2NH₄]³⁺.

Amine 14a (47 mg, 0.0162 mmol) was stirred with the mixture of NHS ester 15a (21 mg, 0.0147 mmol) and Et3N (6 uL, 0.041 mmol) in DCM (4 mL) for 16 h. The solvent was removed in vacuo, and the product 16a was purified on CombiFlash using the system DCM: 20% MeOH in DCM, gradient 0-100%, 40 min. Calculated MW 4188.28, (M+2+18)/3=1402.76, (M+3+18×2)/4=1052.32 Found: MS (ES, pos): 1402.71 [M+2H+NH₄]³⁺, 1052.32 [M+3H+NH₄]⁴⁺.

### Synthesis of LP41-p

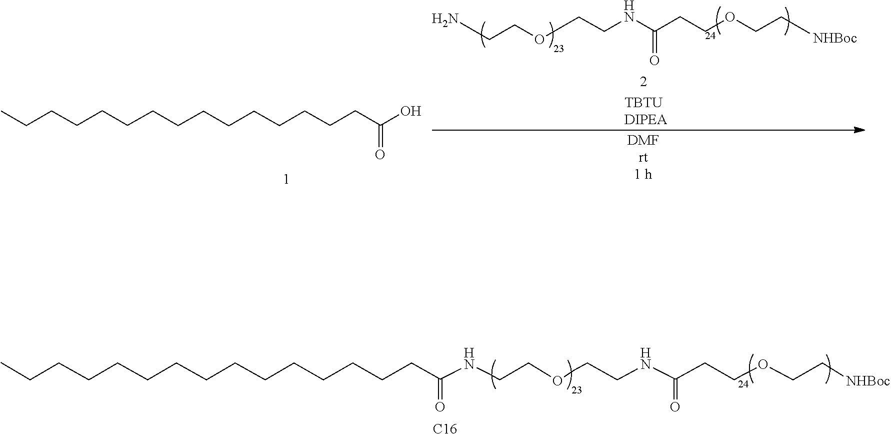

To a solution of compound 1 (40.0 mg), TBTU (50.1 mg), and DIEA (0.098 mL) in DMF was added compound 2 (298 mg) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (10-100% B) over 20-30 min., in which product eluted at 43% B. The product was concentrated under vacuum to provide a white oily residue. LC-MS: calculated [M+H]+ 2539.62 m/z, observed 1287.83 (+2/2, +H2O) m/z.

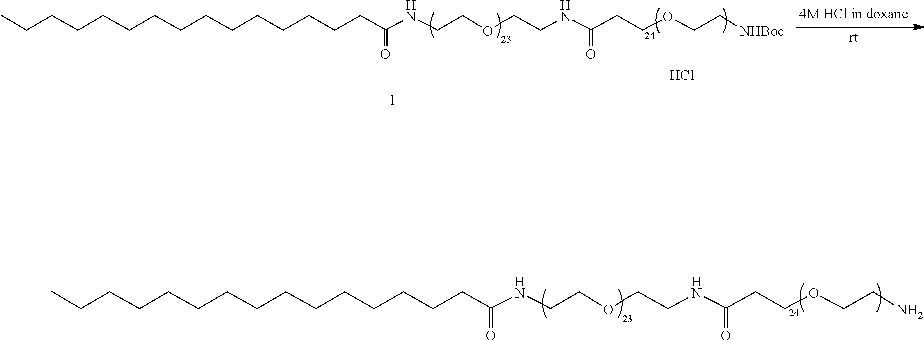

To compound 1 (260 mg) was added 4 M HCl/dioxane (37.4 mg) at room temperature. The reaction was stirred under ambient conditions. The reaction was stirred overnight until full conversion was confirmed via LC-MS. The reaction mixture was azeotroped with PhMe and concentrated under vacuum overnight to provide an oil. LC-MS: calculated [M+H]+ 2439.57 m/z, observed 1220.61 (+2/2) m/z.

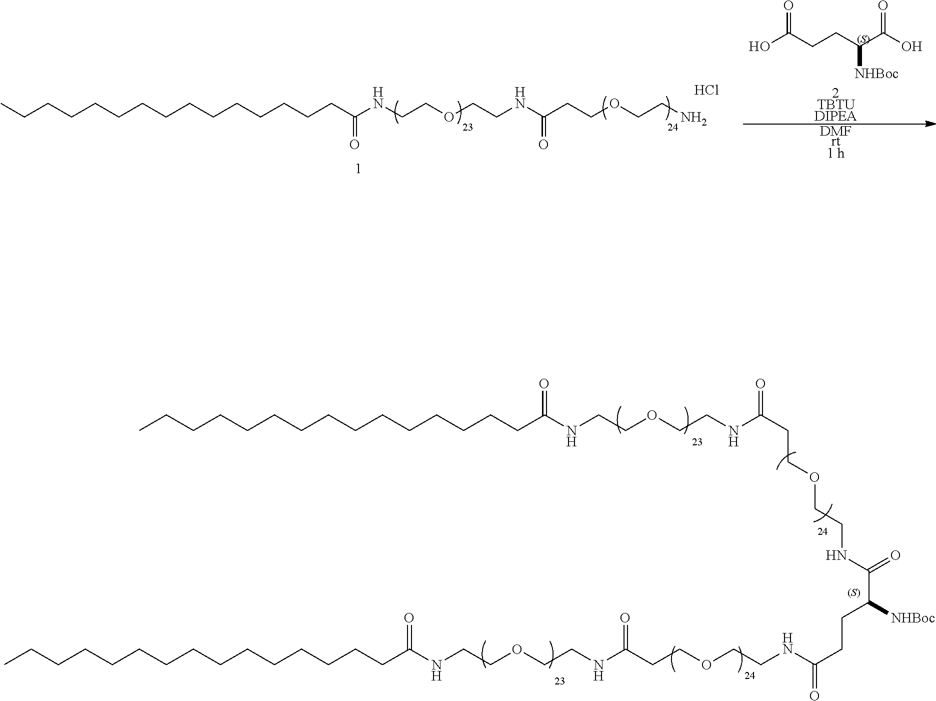

To a solution of compound 1 (253 mg), TBTU (36.1 mg), and DIEA (0.045 mL) in DMF was added compound 2 (11.9 mg) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (10-30, 35, then 100%) over 30 min., in which product eluted at 35% B. The product was concentrated under vacuum to provide a white oily residue. LC-MS: calculated [M+H]+ 5089.22 m/z, observed 1715.43 (+3/3, +H₂O) m/z.

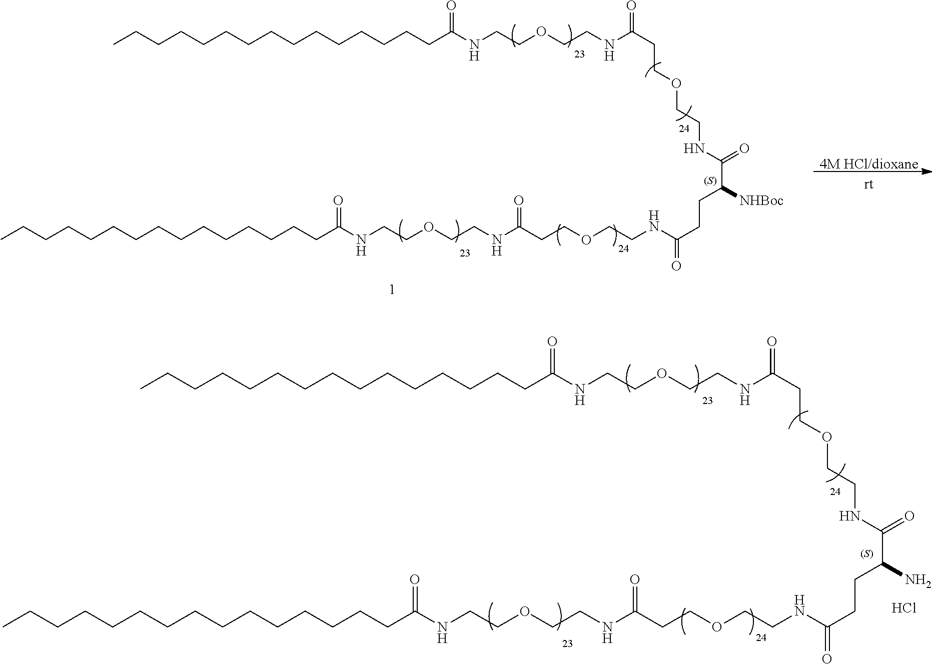

To compound 1 (35.4 mg) was added 4 M HCl/dioxane (2.5 mg) at room temperature. The reaction was stirred under ambient conditions. The reaction was stirred overnight until full conversion was confirmed via LC-MS. The reaction mixture was azeotroped with PhMe/MeOH and concentrated under high-vacuum overnight to provide an oil. LC-MS: calculated [M+H]+ 4989.17 m/z, observed 1676.42 (+HCl, +3/3)m/z.

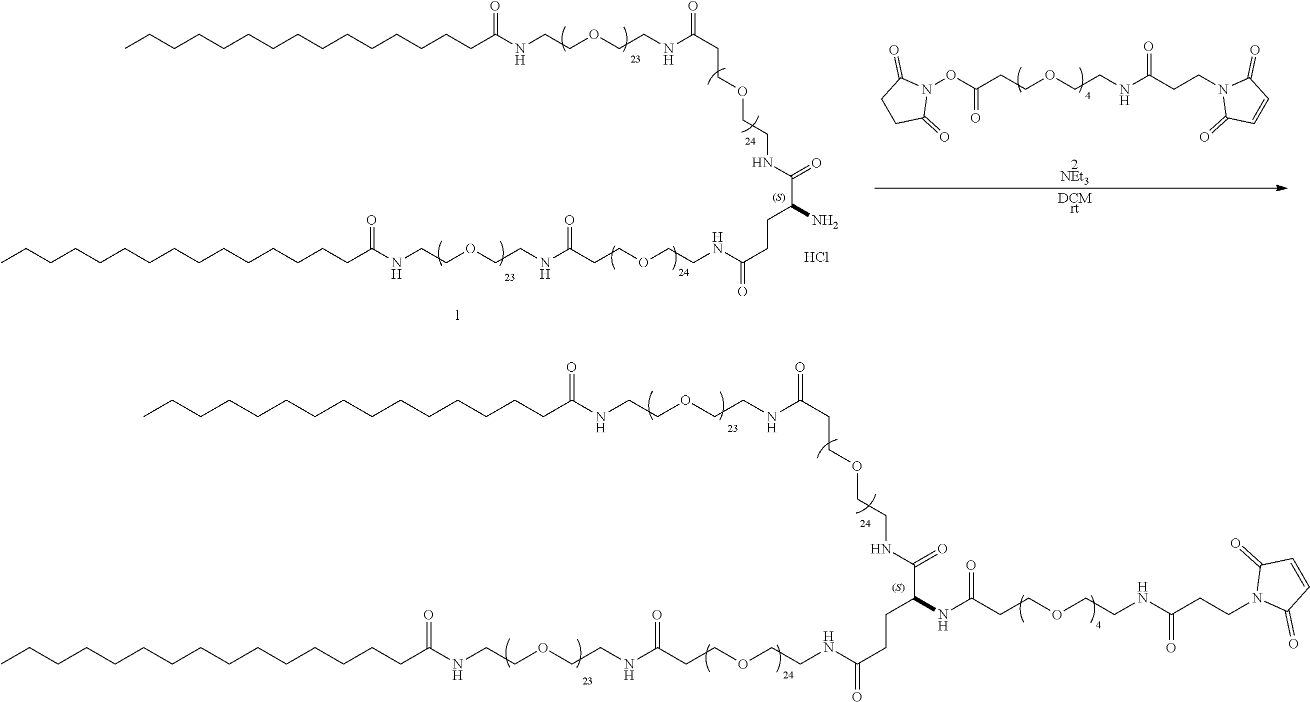

A solution of compound 1 (35 mg) and NEt₃ (0.005 mL) in anhydrous DCM under sparging N₂(g) was prepared at room temperature. Compound 2 (3.2 mg) was then added slowly. The reaction mixture was allowed to stir until full conversion was observed by LC-MS. The reaction mixture was then directly concentrated. The residue was purified by CombiFlash using silica gel as the stationary phase with a gradient of DCM to 20% MeOH/DCM (10 to 30%, 40%, 50%, 70%, then 100% B) over 30 min., in which product eluted at 100% B. LC-MS: calculated [M+H]+ 5837.84 m/z, observed 1079.90 (+5/5) m/z.

### Synthesis of LP42-p

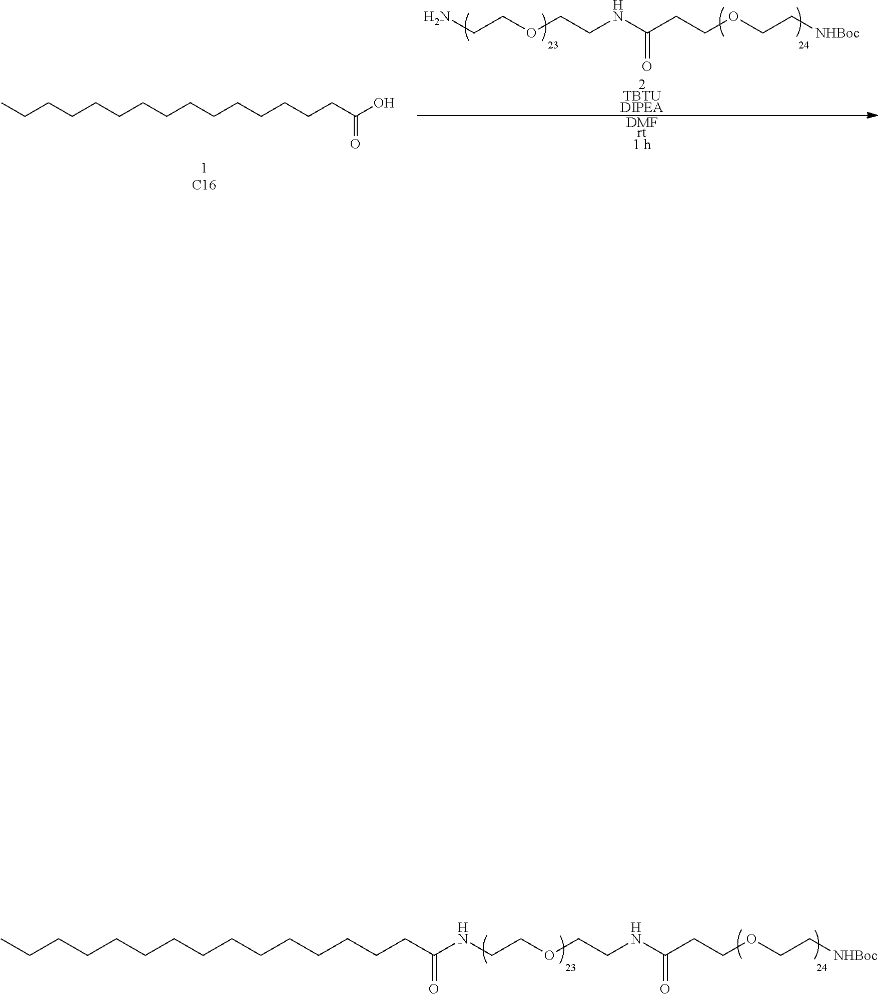

To a solution of compound 1 (40 mg), TBTU (50.1 mg), and DIEA (0.098 mL) in DMF was added compound 2 (298 mg) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (10-100% B) over 20-30 min., in which product eluted at 43% B. The product was concentrated under vacuum to provide a white oily residue. LC-MS: calculated [M+H]+ 2539.62 m/z, observed 1287.83 (+2/2, +H₂O) m/z.

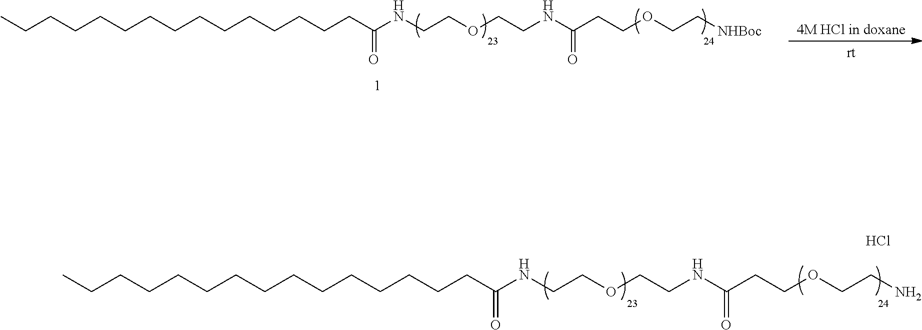

To compound 1 (260 mg) was added 4 M HCl/dioxane (37.4 mg) at room temperature. The reaction was stirred under ambient conditions. Reaction was stirred overnight until full conversion was confirmed via LC-MS. The reaction mixture was azeotroped with PhMe and concentrated under vacuum overnight to provide an oil. LC-MS: calculated [M+H]+ 2439.57 m/z, observed 1220.61 (+2/2) m/z.

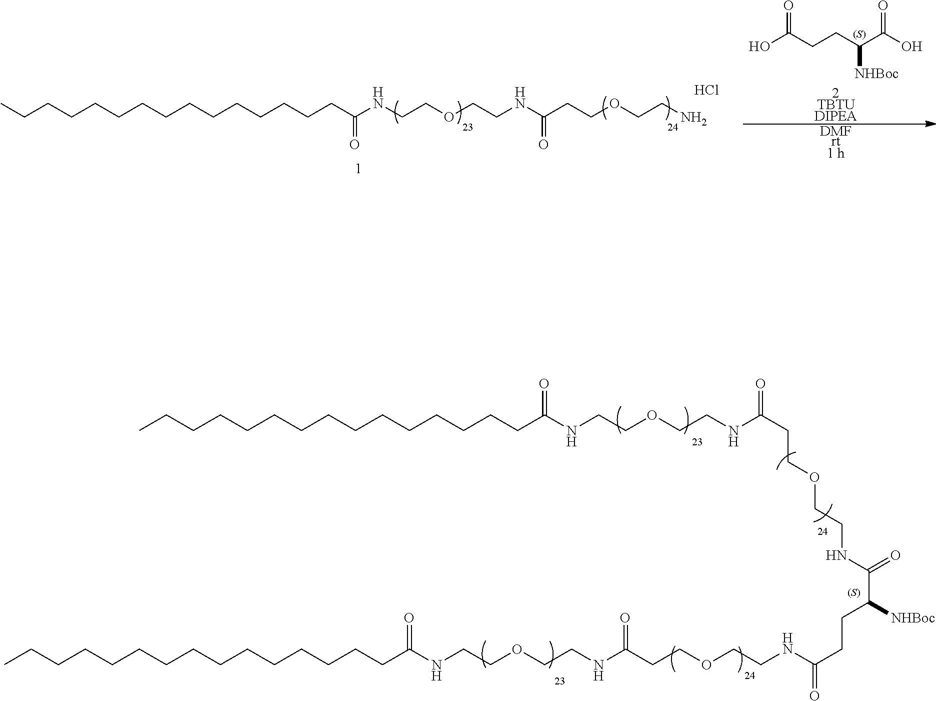

To a solution of compound 1 (253 mg), TBTU (36.1 mg), and DIEA (0.045 mL) in DMF was added compound 2 (11.9 mg) under ambient conditions. Reaction was stirred until full conversion was observed by LC-MS. The reaction mixture was then directly concentrated for isolation. The residue was purified by CombiFlash using silica gel as the stationary phase with a gradient of DCM to 20% MeOH in DCM (10-30, 35, then 100%) over 30 min., in which product eluted at 35% B. The product was concentrated under vacuum to provide a white oily residue. LC-MS: calculated [M+H]+ 5089.22 m/z, observed 1715.43 (+3/3, +H₂O) m/z.

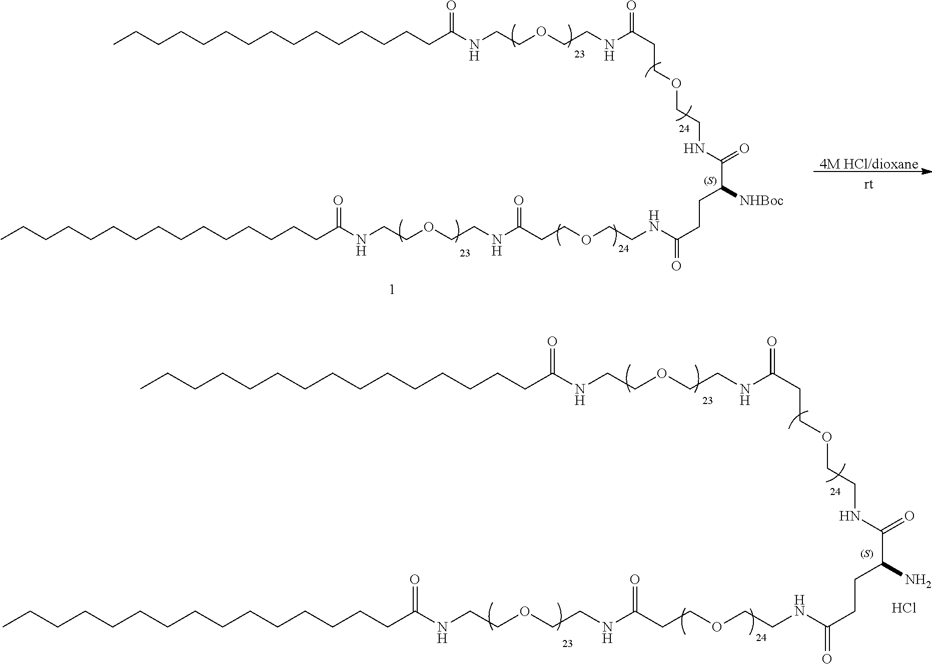

To compound 1 (28.2 mg) was added 4 M HCl/dioxane (2.0 mg) at room temperature. The reaction was stirred under ambient conditions. Reaction was stirred overnight until full conversion was confirmed via LC-MS. The reaction mixture was azeotroped with PhMe/MeOH and concentrated under high-vacuum overnight to provide an oil. LC-MS: calculated [M+H]+ 4989.17 m/z, observed 1000.21 (+5/5) m/z.

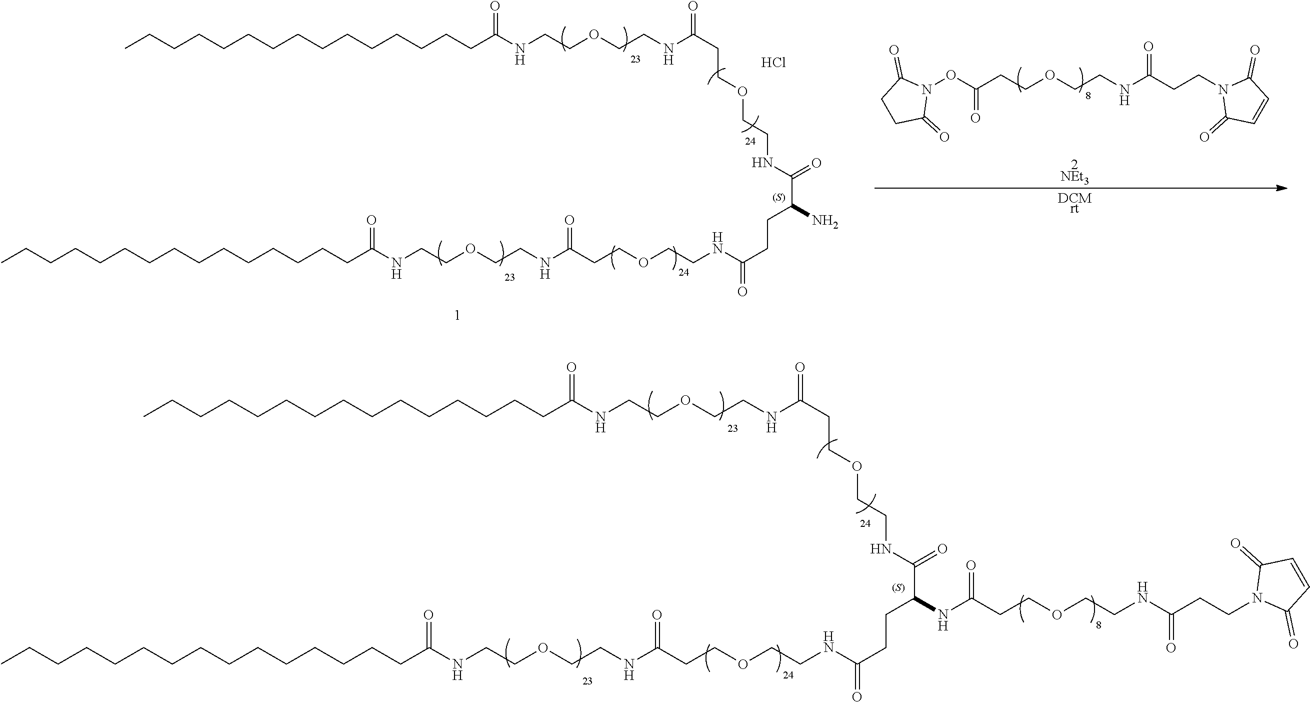

A solution of compound 1 (27.9 mg) and NEt₃ (0.004 mL) in anhydrous DCM under sparging N₂(g) was prepared at room temperature. Compound 2 (3.4 mg) was then added slowly. The reaction mixture was allowed to stir until full conversion was observed by LC-MS. The reaction mixture was allowed to stir until full conversion was observed by LC-MS. The reaction mixture was then directly concentrated. The residue was purified by CombiFlash using silica gel as the stationary phase with a gradient of DCM to 20% MeOH/DCM (25 to 50%, then 100% B) over 30 min., in which product eluted at 100% B after 5 min. at 100% B. LC-MS: calculated [M+H]+ 5563.44 m/z, observed 946.45 (+6/6, +water) m/z.

### Synthesis of LP43-p

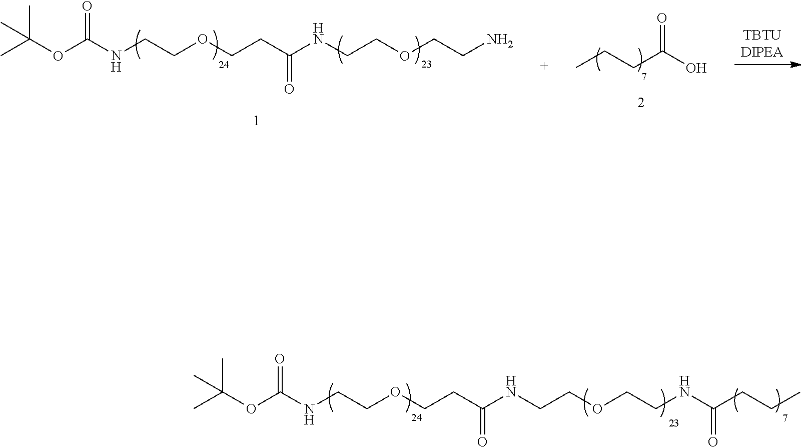

To a solution of compound 1 (3.0 g, 1.303 mmol, 1.0 equiv.), compound 2 (0.401 g, 1.564 mmol, 1.2 equiv.) and diisopropylethylamine (0.681 mL, 3.91 mmol, 3.0 equiv.) in DMF (20 mL) was added TBTU (0.502 g, 1.564 mmol, 1.2 equiv.) at room temperature. The reaction was kept at room temperature for 3 hrs. The reaction mixture was concentrated. The product was purified by CombiFlash and was eluted with 12-18% methanol in dichloromethane. Structure confirmed by H-NMR.

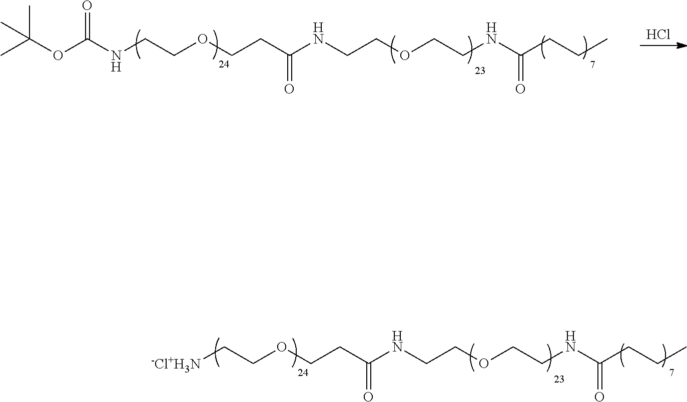

To solid of compound 1 (2060 mg, 0.811 mmol, 1.0 equiv.) was added HCl solution in dioxane (4.055 mL, 16.219 mmol, 20 equiv.) at room temperature. The reaction was kept at room temperature for 1 hr and the solvent was concentrated. The product was used directly without further purification. Structure confirmed by H-NMR.

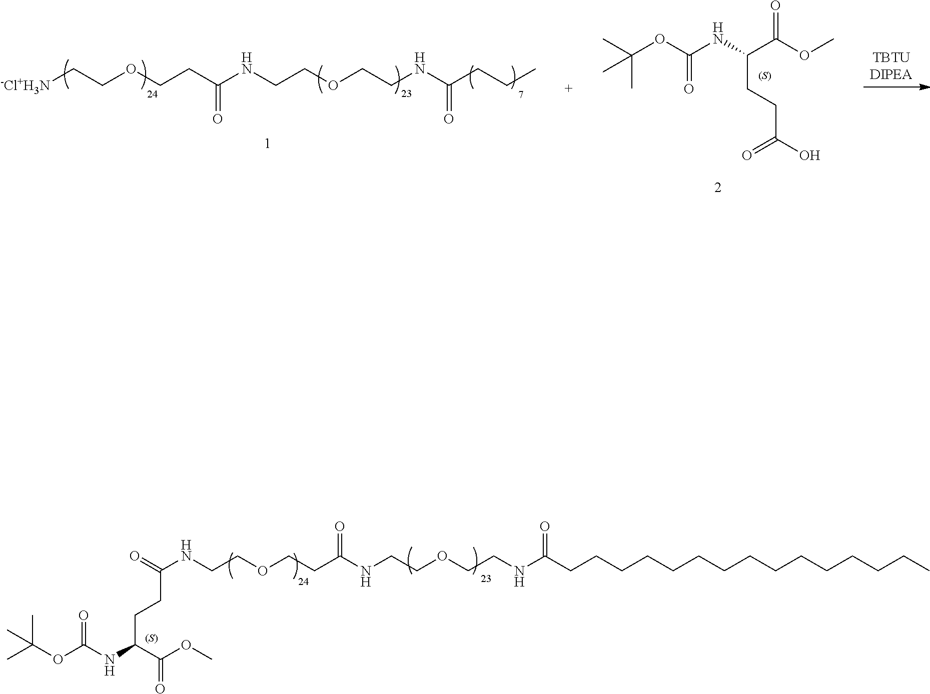

To a solution of compound 1 (2030 mg, 0.819 mmol, 1.0 equiv.), compound 2 (257 mg, 0.983 mmol, 1.2 equiv.) and diisopropylethylamine (0.428 mL, 2.459 mmol, 3.0 equiv.) in anhydrous DMF (10 mL) was added TBTU (315 mg, 0.983 mmol, 1.2 equiv.) at room temperature. The reaction was kept at room temperature overnight. The reaction mixture was concentrated. The product was purified by CombiFlash and was eluted with 12-20% methanol in dichloromethane. LC-MS: [M+2H]/2, calculated 1341.84, found 1342.69.

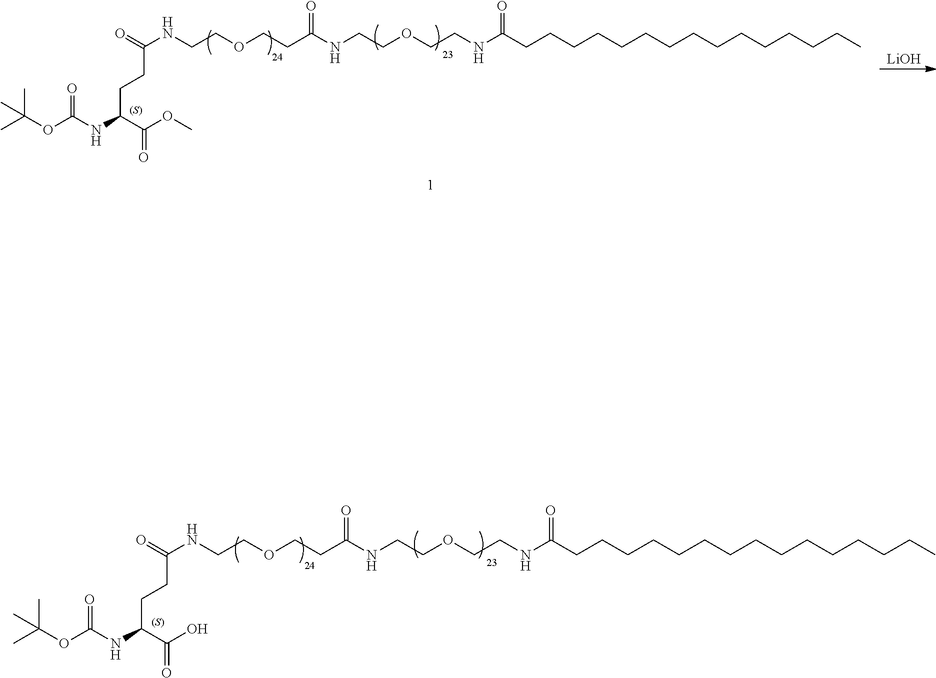

To a solution of compound 1 (1430 mg, 0.530 mmol, 1.0 equiv.) in THE (20 mL) and water (20 mL) was added lithium hydroxide (63.8 mg, 2.664 mmol, 5.0 equiv.) at room temperature. The reaction was kept at room temperature for 3 hrs. The reaction was quenched with HCl solution and the pH was adjusted to 3.0. The aqueous phase was extracted with DCM (3×20 mL). The organic phase was combined, dried over Na₂SO₄, and concentrated. The product was used directly without further purification. LC-MS: [M+2H]/2 calculated 1334.83, found 1335.49.

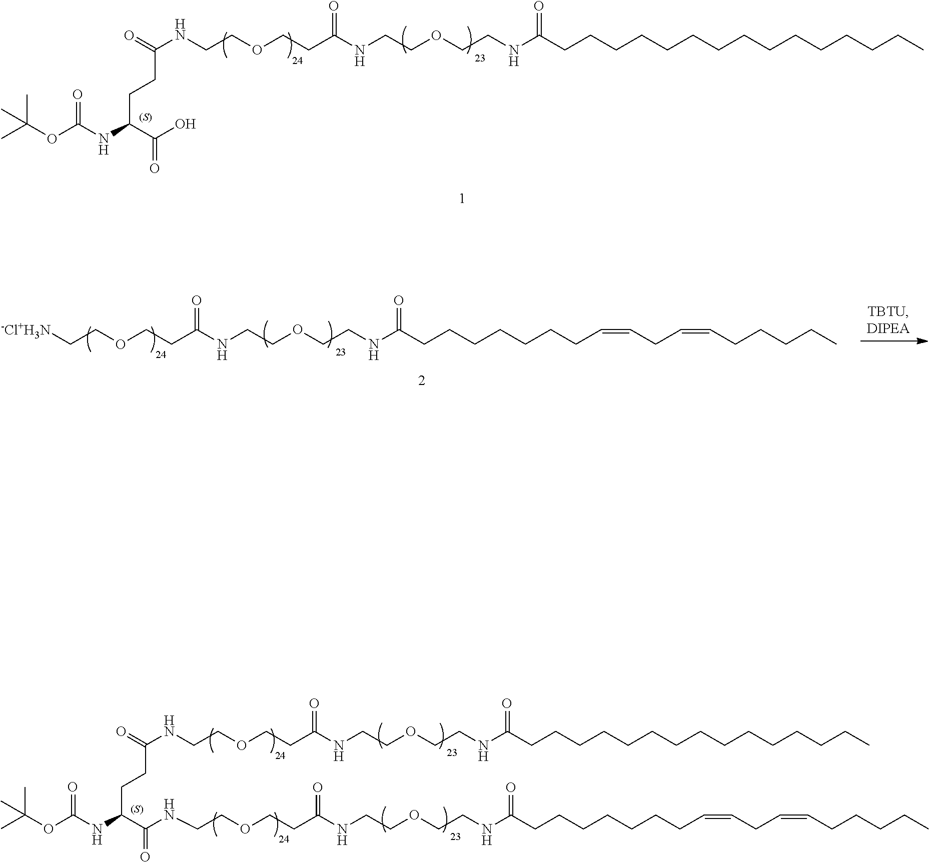

To a solution of compound 1 (110 mg, 0.0412 mmol, 1.0 equiv.), compound 2 (103 mg, 0.0412 mmol, 1.00 equiv.) and diisopropylethylamine (0.022 mL, 0.123 mmol, 3.0 equiv.) in DMF (2 mL) was added TBTU (15.9 mg, 0.0495 mmol, 1.2 equiv.) at room temperature. The reaction was kept at room temperature overnight. The reaction mixture was concentrated. The product was purified by CombiFlash and was eluted with 16-20% methanol in dichloromethane. LC-MS: [M+5H]/5 calculated 1023.44, found 1024.00.

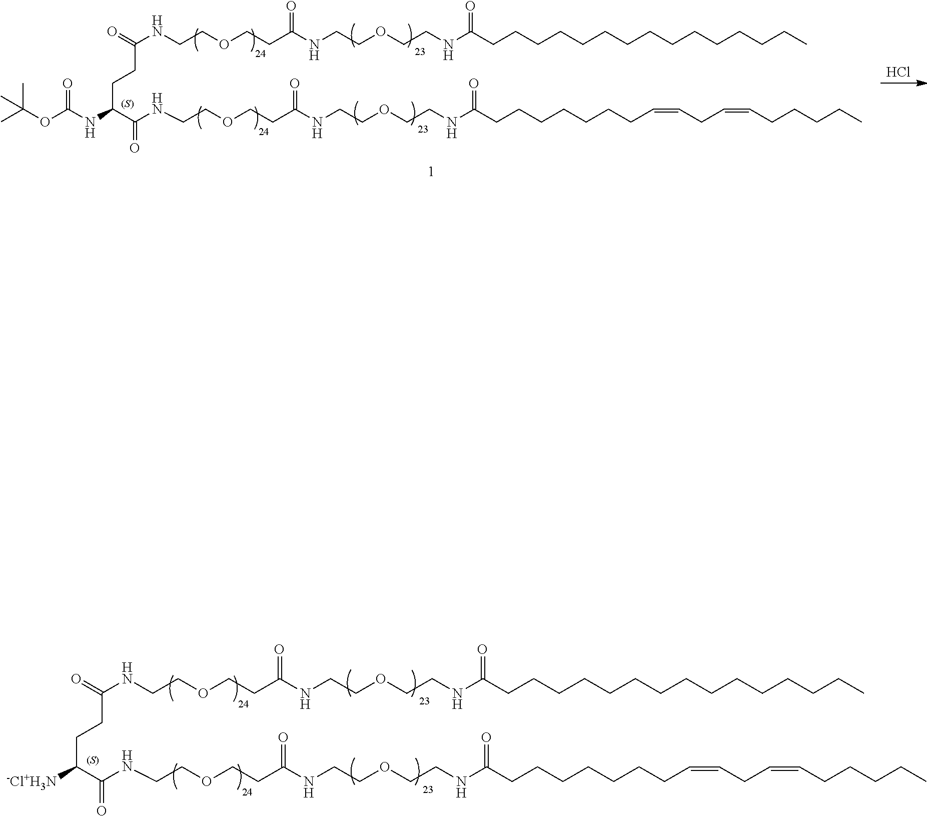

To compound 1 (84 mg, 0.0164 mmol, 1.0 equiv.) was added 4M HCl in dioxane (0.205 mL, 0.0821 mmol, 50 equiv.) at room temperature. The reaction was kept at room temperature for 1 hr. The reaction mixture was concentrated. The product was used directly without further purification. LC-MS: [M+5H]/5 calculated 1003.44, found 1004.07.

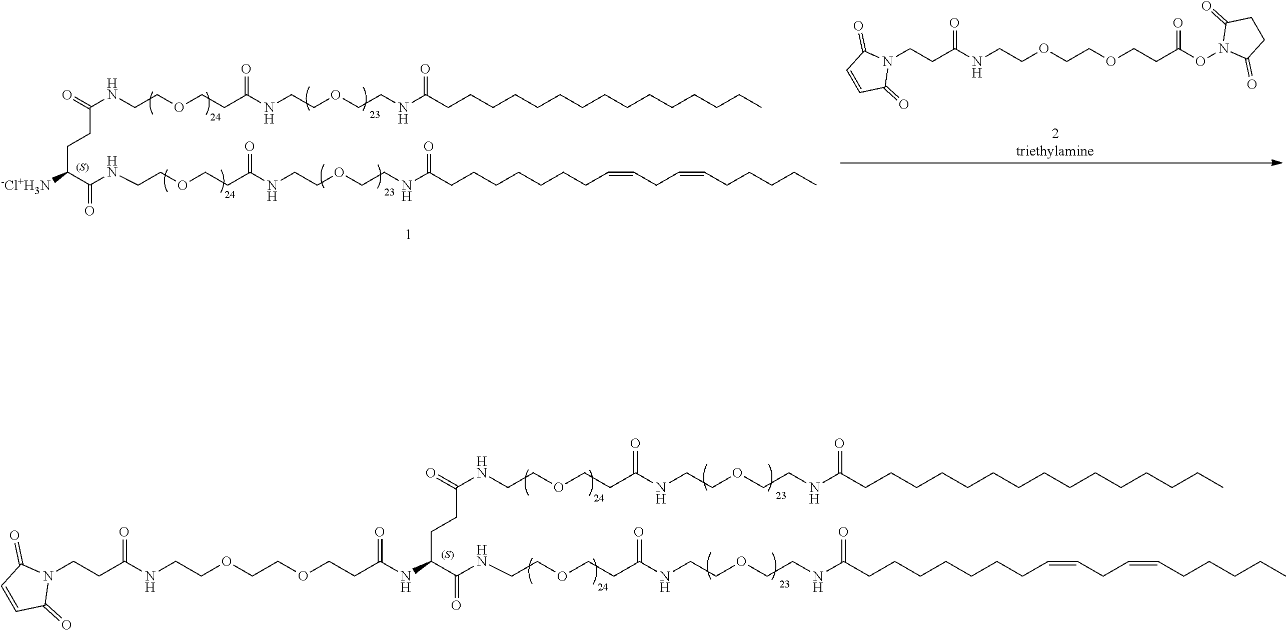

To a solution of compound 1 (125 mg, 0.0247 mmol, 1.0 equiv.) and compound 2 (116 mg, 0.0272 mmol, 1.10 equiv.) in anhydrous DCM (2 mL) was added triethylamine (0.017 mL, 0.123 mmol, 5.0 equiv.) at room temperature. The reaction was kept at room temperature overnight and the solvent was concentrated. The product was separated by CombiFlash and was eluted with 18-20% methanol in dichloromethane. LC-MS: [M+5H]/5 calculated 1065.46, found 1066.13.

### Synthesis of LP44-p

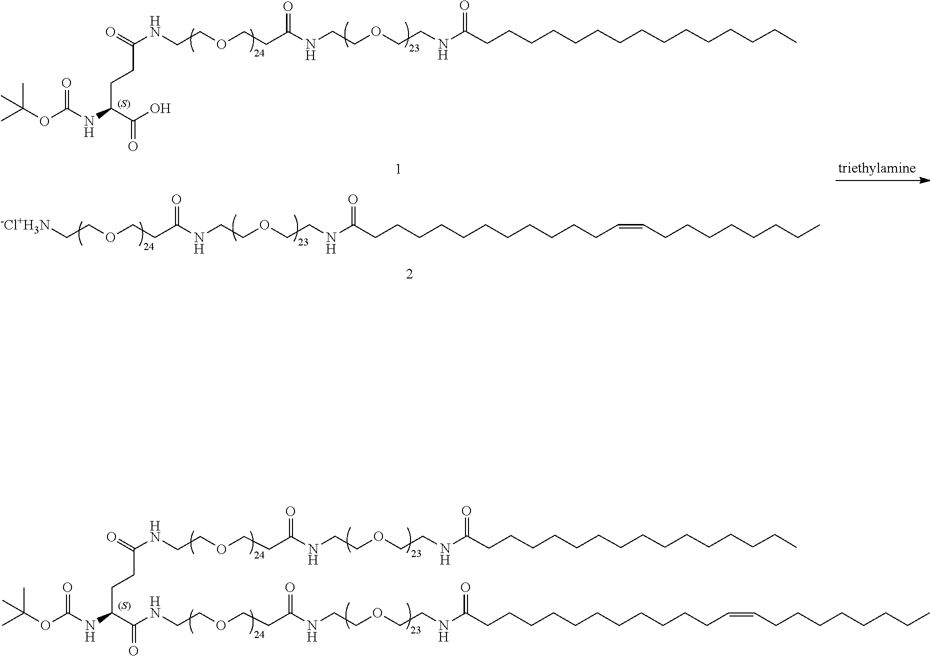

Compound 1 was synthesized as shown in the steps in the synthesis of LP43, above. To a solution of compound 1 (135 mg, 0.0506 mmol, 1.0 equiv.), compound 2 (129 mg, 0.0506 mmol, 1.00 equiv.) and diisopropylethylamine (0.026 mL, 0.151 mmol, 3.0 equiv.) in DMF (2 mL) was added TBTU (19.5 mg, 0.0607 mmol, 1.2 equiv.) at room temperature. The reaction was kept at room temperature overnight. The reaction mixture was concentrated. The product was purified by CombiFlash and was eluted with 12-20% methanol in dichloromethane. LC-MS: [M+5H]/5 calculated 1035.06, found 1035.40.

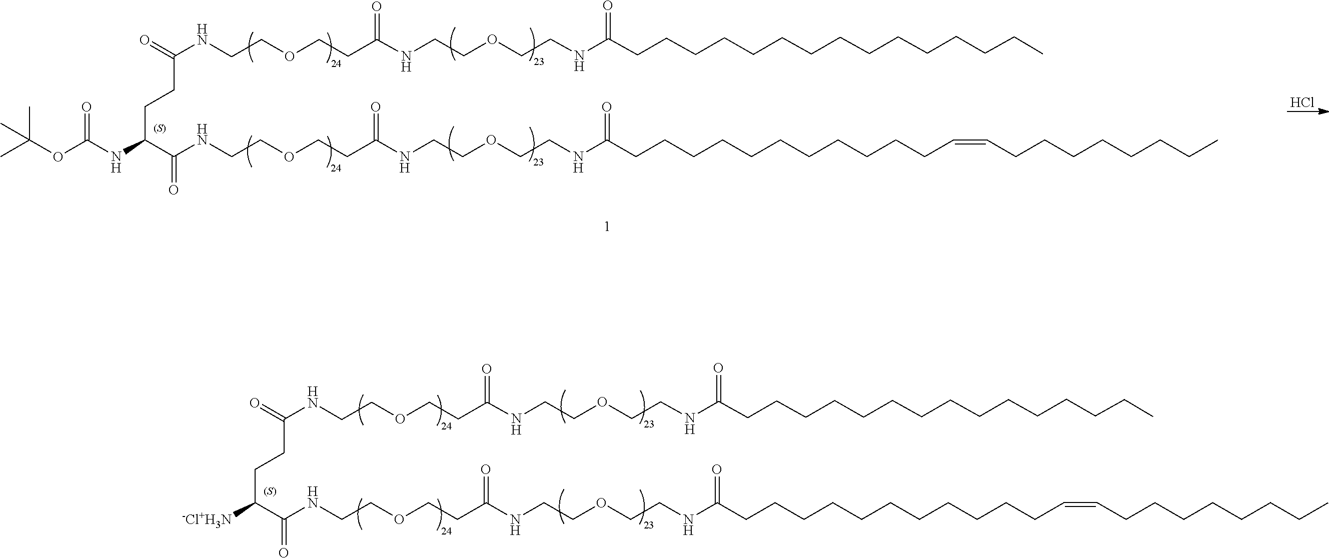

To compound 1 (100 mg, 0.0193 mmol, 1.0 equiv.) was added 4M HCl in dioxane (0.242 mL, 0.966 mmol, 50 equiv.) at room temperature. The reaction was kept at room temperature for 1 hr. The reaction mixture was concentrated. The product was used directly without further purification. LC-MS: [M+5H]/5 calculated 1015.05, found 1015.71.

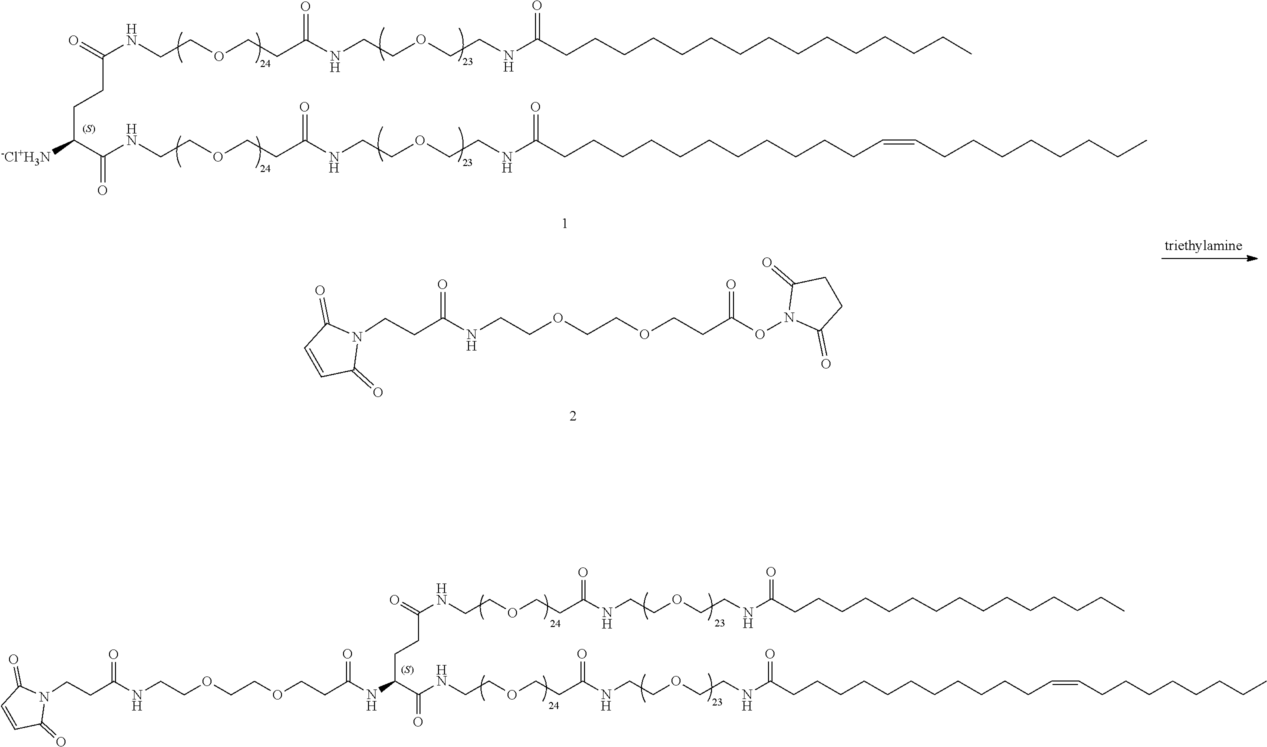

To a solution of compound 1 (95 mg, 0.0186 mmol, 1.0 equiv.) and compound 2 (8 mg, 0.0186 mmol, 1.0 equiv.) in anhydrous DCM (2 mL) was added triethylamine (0.013 mL, 0.0930 mmol, 5.0 equiv.) at room temperature. The reaction was kept at room temperature overnight and the solvent was concentrated. The product was separated by CombiFlash and was eluted with 12-20% methanol in dichloromethane. LC-MS: calculated [M+5H]/5 1077.74, found 1079.

### Synthesis of LP45-p

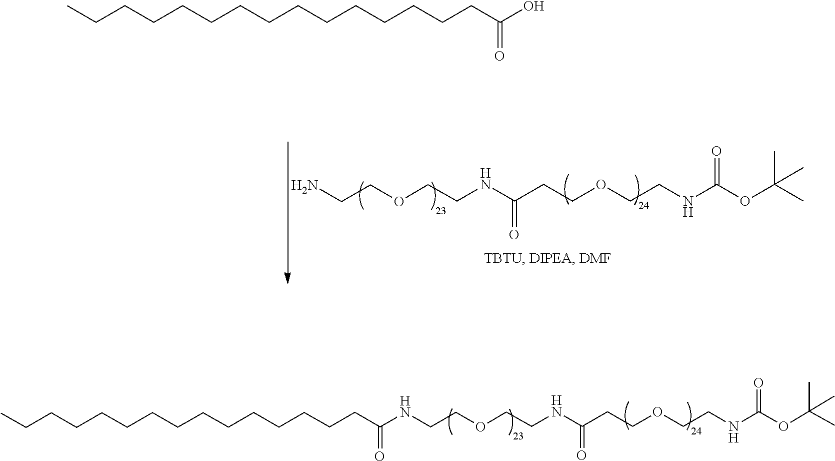

To palmitic acid acid (30 mg, 0.1170 mmol) in a solution of DMF (2.0 mL) with Boc-PEG₄₇-NH₂ (269 mg, 0.1170 mmol) was added TBTU (45.1 mg, 0.1404 mmol) and DIPEA (60 uL). After stirring the suspension overnight, water was added and the product extracted using DCM:20% TFE and dried over Na₂SO₄. After filtration, the solvent was concentrated to dryness and the crude product was purified by flash chromatography (DCM:20% MeOH).

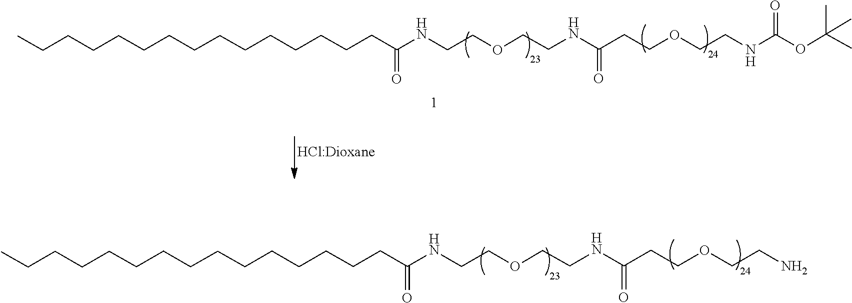

To compound 1 was added 2 mL of 4N HCl:Dioxane and stirred under anhydrous conditions until determined complete by LC-MS: calculated [M+H]+for C₁₆-PEG₄₇-NH_{2 2301} m/z, found 2302.

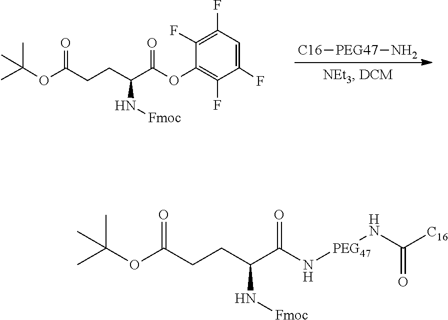

To Fmoc-Glu(OtBu)-Opfp (50 mg, 0.0845 mmol) was stirred in a solution of C₁₆-PEG₄₇-NH₂ (206 mg, 0.0.0845 mmol) was added NEt₃ (29 uL), while stirring in DCM (5.0 mL). After stirring the suspension until determined complete, the solvent was concentrated to dryness and the crude product was purified by FC (DCM:20% MeOH).

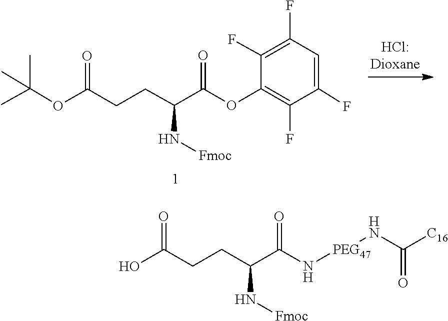

To compound 1 was added 2 mL of 4N HCl:Dioxane and stirred under anhydrous conditions until determined complete by LC-MS: Calculated 2866.0 [M+H]+found 2867.

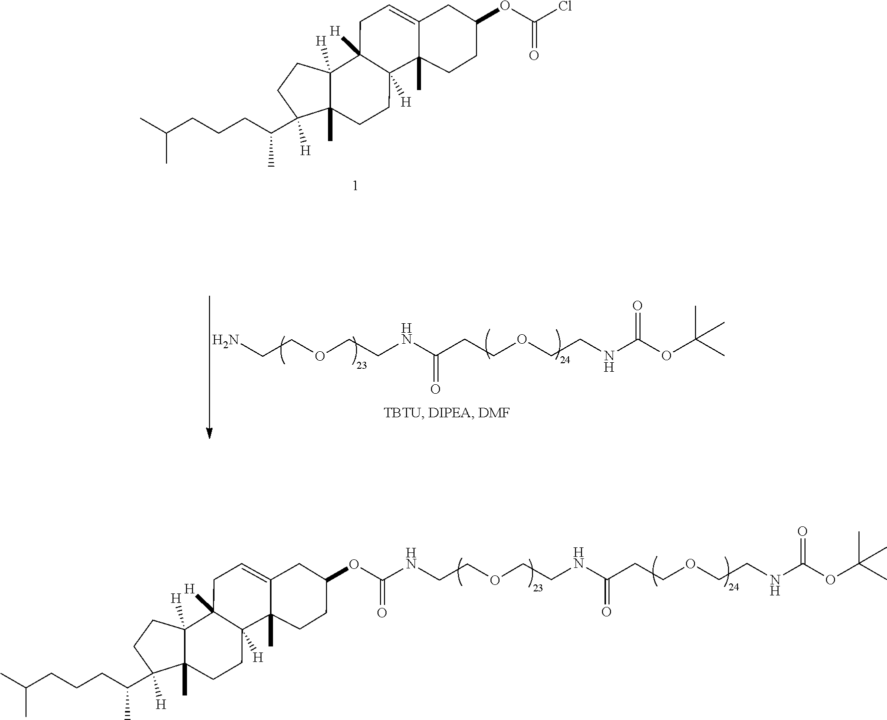

In a solution of Boc-PEG₄₇-NH₂ (269 mg, 0.1170 mmol) with TBTU (45.1 mg, 0.1404 mmol) and DIPEA (60 uL), while stirring in DMF (2.0 mL) was added compound 1 (30 mg, 0.1170 mmol). After stirring the suspension overnight, water was added and the product was extracted using DCM:20% TFE and dried over Na₂SO₄. After filtration, the solvent was concentrated to dryness and the crude product was purified by flash chromatography (DCM:20% MeOH). Calculated [M+H]+for 2614.32 m/z, found 2615.32.

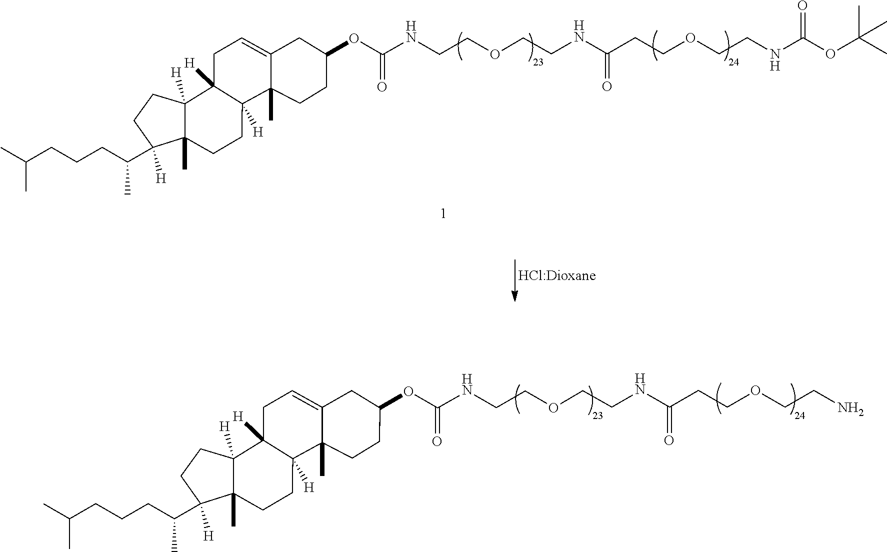

To compound 1 was added 2 mL of 4N HCl:Dioxane and stirred under anhydrous conditions until determined complete. The product was used in the next step without further purification.

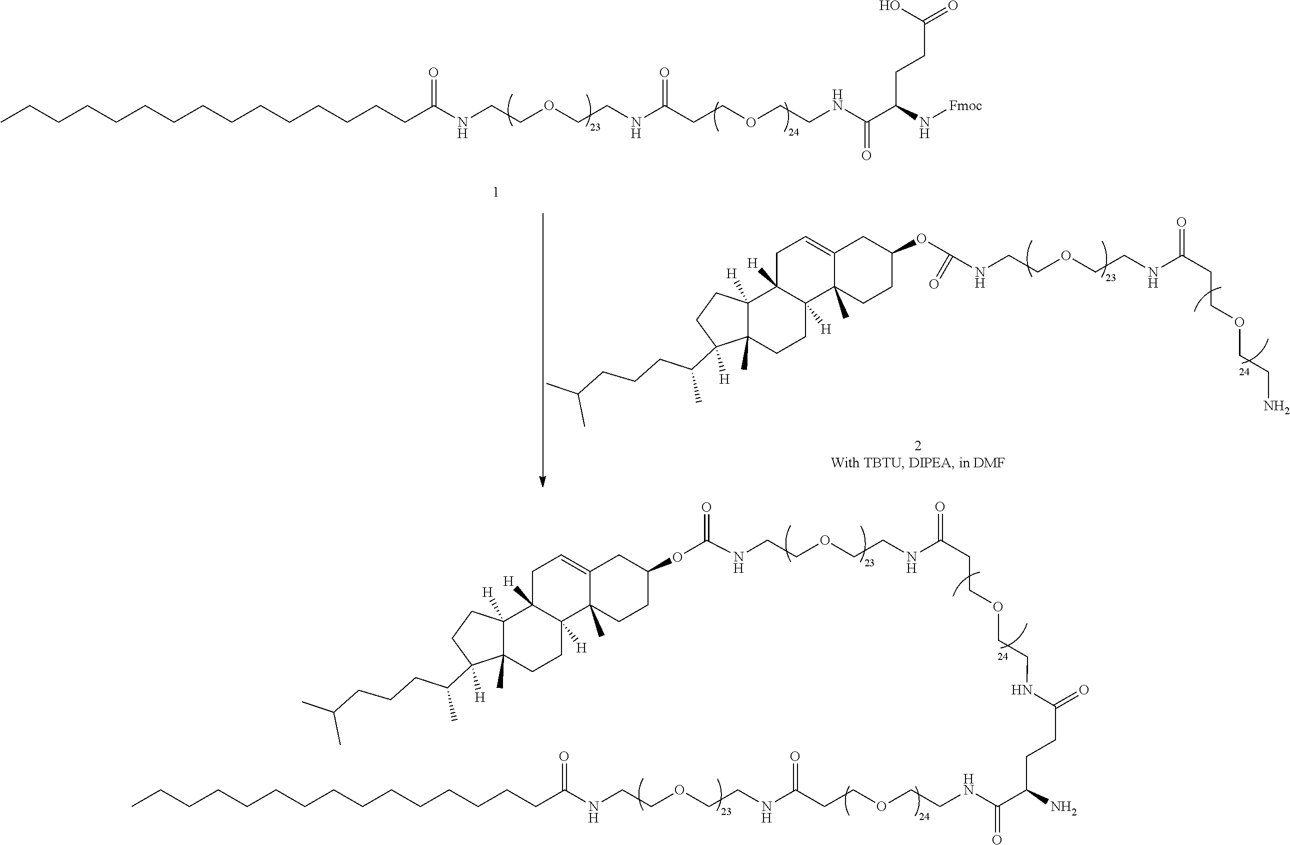

To compound 1 (100 mg, 0.0375 mmol) in a solution of DMF (5.0 mL) with compound 2 (98 mg, 0.1914 mmol) was added TBTU (14.4 mg, 0.045 mmol) and DIPEA (20 uL). After stirring the suspension overnight, water was added and extracted using DCM:20% TFE and dried over Na₂SO₄. After filtration, the solvent was concentrated to dryness and the crude product was purified by FC (DCM:20% MeOH). To this was added 2 mL of 4N HCl:Dioxane and stirred under anhydrous conditions until determined complete by LC-MS: calculated [M+H]+for 5134.26 m/z, found 5135.

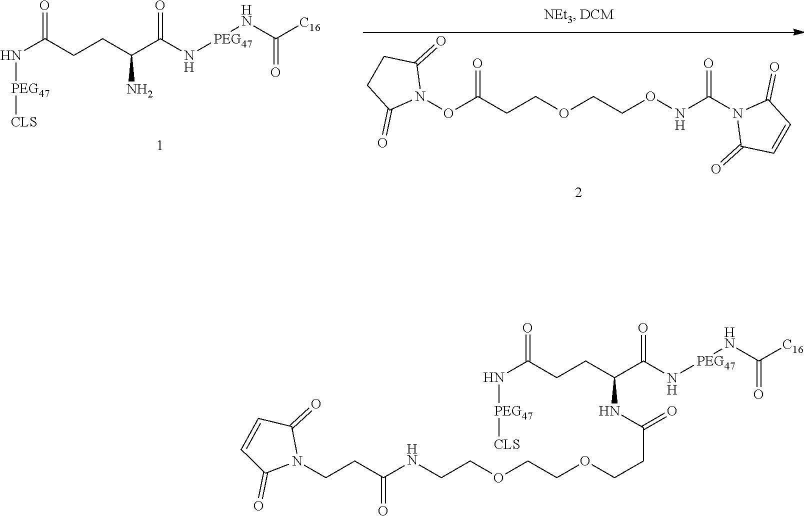

To a solution of compound 2 (10 mg, 0.0235 mmol, 1.0 equiv.) and compound 1 (120 mg, 0.0235 mmol, 1.0 equiv.) in anhydrous DCM (2 mL) was added triethylamine (17 uL, 0.1175 mmol, 5.0 equiv.) at room temperature. The reaction was kept at room temperature overnight and the solvent was concentrated. The product was separated by CombiFlash and was eluted with 10-17% methanol in dicholoromethane. LC-MS: calculated [M+6H]+5474.38, found 5475.01.

### Synthesis of LP47-p

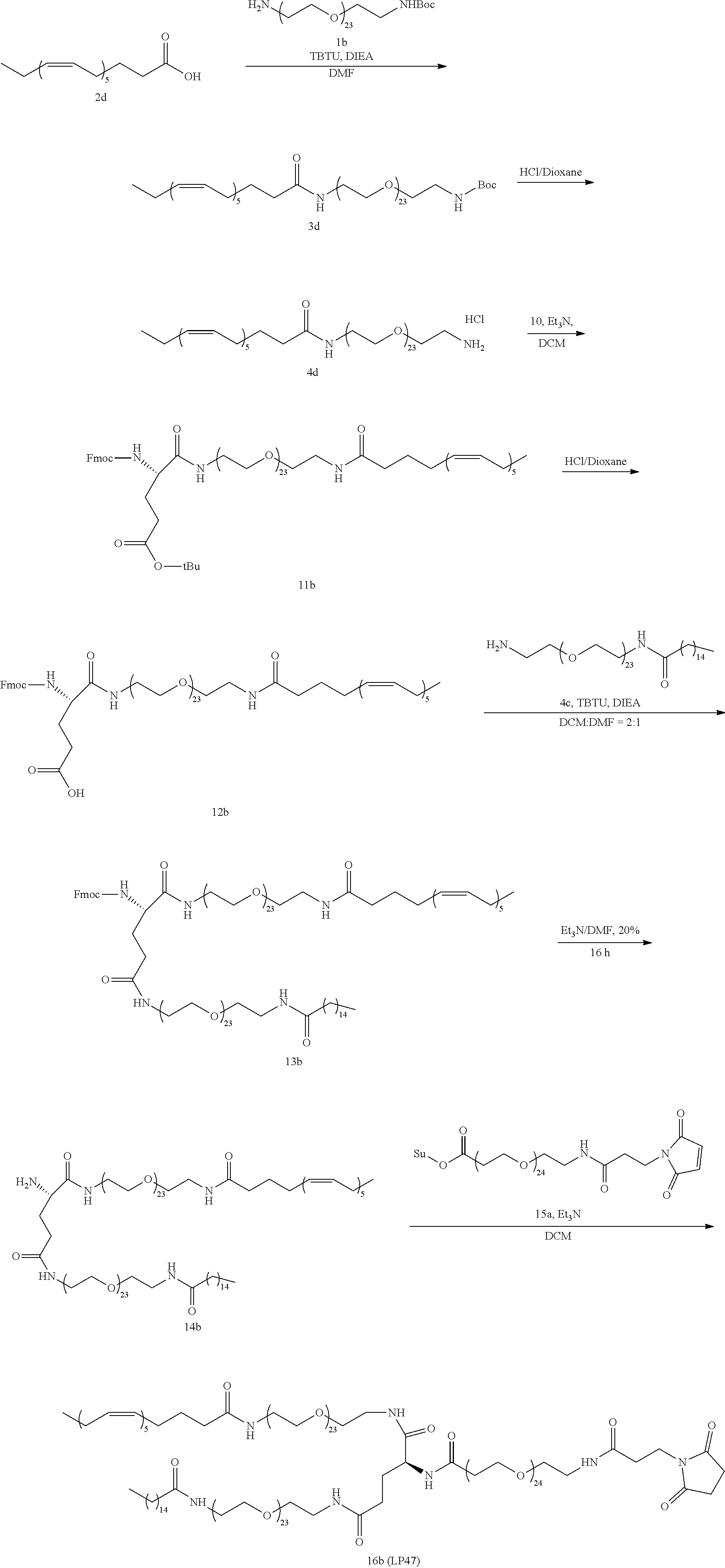

Solid TBTU (50 mg, 0.156 mmol) was added to a solution of Boc-protected Peg23-amine 1b (Quanta Biodesign Limited, 150 mg, 0.13 mmol), eicosapentaenoic acid 2d (39 mg, 0.13 mmol), and DIEA (68 uL mL, 0.39 mmol) in DMF (9 mL). The reaction mixture was sonicated to dissolve solids and stirred for 16 h at RT. The solvent was removed in high vacuo, toluene was evaporated twice from the residue, the residue was dissolved in chloroform (50 mL), washed with NaHCO₃(2×10 mL) and brine (10 mL). Product was dried (Na₂SO₄), concentrated in vacuo, and purified on CombiFlash (SiO₂) using the system DCM: 20% MeOH in DCM, gradient 0-80%, 20 min. The Boc group was removed with 4M solution of HCl in dioxane to obtain hydrochloride salt 4d. Calculated MW 1357.76, (M+2)/2=679.88 Found: MS (ES, pos): 1358.29 [M+H]+, 679.77 [M+2H]2+.

Hydrocloride salt 4d (167 mg, 0.123 mmo) was stirred with pentafluorophenyl ester 10 (73 mg, 0.123 mmol) and Et3N (43 uL, 0.31 mmol) in DCM (5 mL) for 2 h. The solvent was removed in vacuo, the residue was mixed with SiO₂ (1 g) and loaded on CombiFlash. The product 11b was purified using the system DCM: 20% MeOH in DCM, gradient 0-50%, 25 min. Yield 169 mg. Calculated MW 1765.23, M+18=1783.23, (M+1+18)/2=892.12 Found: MS (ES, pos): 1782.78 [M+NH₄]+, 891.97 [M+H+NH₄]2+.

The product 11b was treated with HCl in dioxane as 11a in order to obtain free acid 12b and directly used in the next step. Calculated MW 3002.84, (M+2×18)/2=1519.42, (M+3×18)/3=1018.95. Found: MS (ES, pos): 1519.39 [M+2NH₄]2+, 1019.17 [M+H+2NH₄] 3+.

The derivative 12b (47 mg, 0.028 mmol) was stirred with hydrochloride 4c (42 mg, 0.03 mmol), TBTU (11 mg, 0.034 mmol) and DIEA (18 uL, 0.1 mmol) in DCM:DMF=1:1 (8 mL) for 3 h. The solvent was removed in vacuo, toluene was evaporated 2 times from the residue, and the solid was suspended in CHCl₃ (50 mL). The suspension was washed twice with 2% NaHCO₃ and brine. Following concentration in vacuo the product 13b was purified on CombiFlash (DCM: 20% MeOH in DCM, gradient 0-70%, 35 min.).

The product 13b (49 mg, 0.0162 mmol) was stirred with Et3N in DMF (20%, 3 mL) for 16 h, the solvent with Et3N was removed in vacuo, toluene was evaporated 3 times from the residue to obtain deprotected amine 14b, which was used directly in the next step.

Amine 14b (45 mg, 0.0162 mmol) was stirred with the mixture of NHS ester 15a (21 mg, 0.0147 mmol) and Et3N (6 uL, 0.041 mmol) in DCM (4 mL) for 16 h. The solvent was removed in vacuo, and the product 16b was purified on CombiFlash using the system DCM: 20% MeOH in DCM, gradient 0-100%, 40 min. Calculated MW 4060.07, (M+3×18)/3=1371.36, (M+4×18)/4=1033.02 Found: MS (ES, pos): 1371.76 [M+3NH₄]3+1033.70 [M+4NH₄]⁴⁺.

### Synthesis of LP48-p

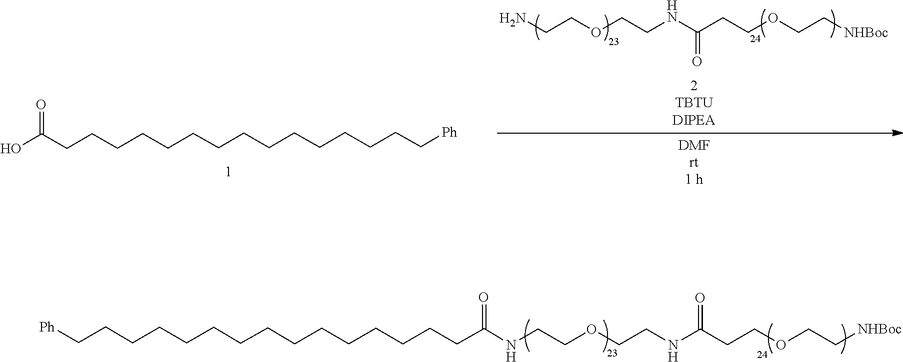

To a solution of compound 1 (27.5 mg), TBTU (26.6 mg), and DIEA (0.022 mL) in DMF was added compound 2 (173 mg) under ambient conditions. The reaction was stirred until full conversion was observed by LC-MS. The reaction mixture was then directly concentrated for isolation. The residue was purified by CombiFlash using a 12-g column of silica gel as the stationary phase with a gradient of DCM to 20% MeOH in DCM (10-100%) over 20 min., in which product eluted at 66% B. The product was concentrated under vacuum to provide a white oily residue. LC-MS: calculated [M+H]+ 2615.65 m/z, observed 1326.52 (+2/2, +H₂O) m/z.

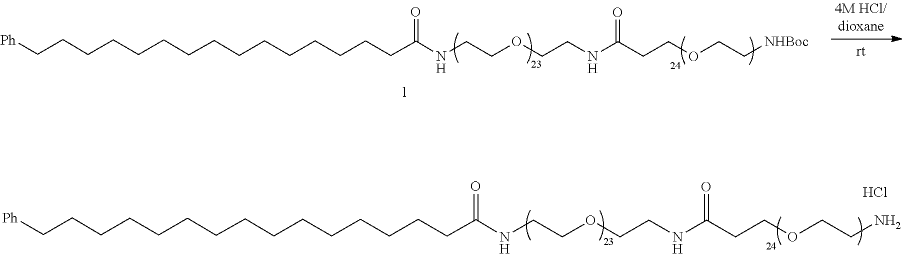

To compound 1 (56.7 mg) was added 4 M HCl/dioxane (7.9 mg) at room temperature. The reaction was stirred under ambient conditions. Reaction was stirred overnight until full conversion was confirmed via LC-MS. The reaction mixture was azeotroped with PhMe/MeOH and concentrated under high-vacuum overnight to provide a white solid. LC-MS: calculated [M+H]+ 2515.60 m/z, observed 1259.91 (+2/2) m/z.

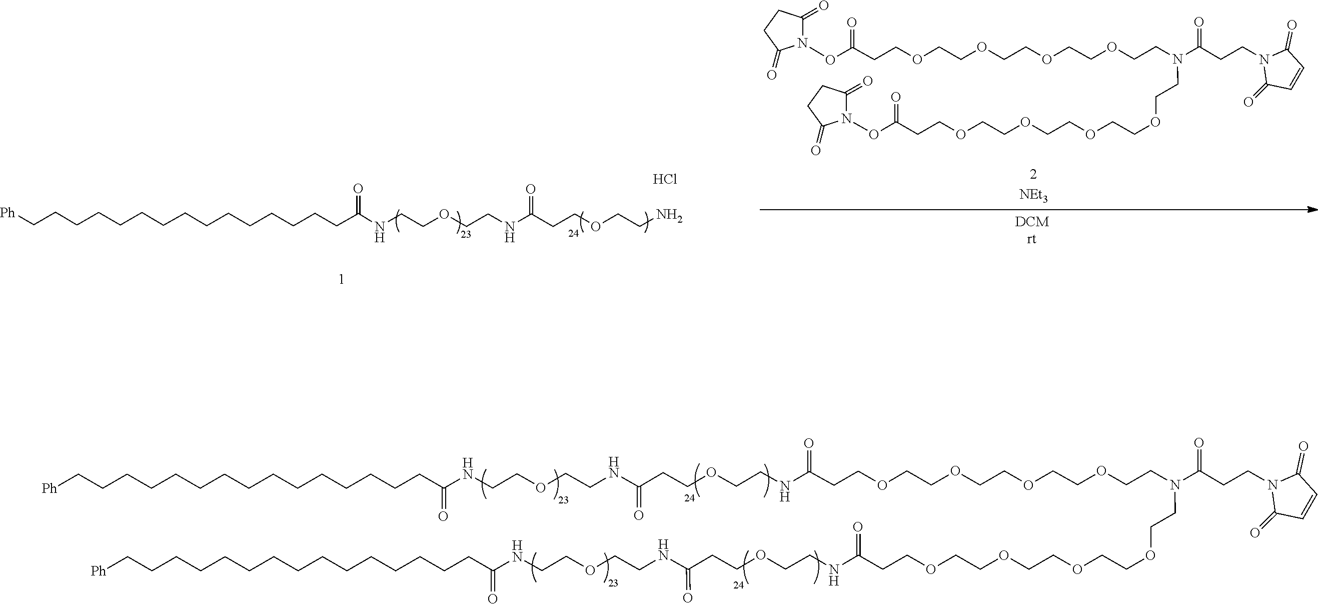

A solution of compound 1 (55.4 mg) and NEt₃ (0.015 mL) in anhydrous DCM under sparging N₂(g) was prepared at room temperature. Compound 2 (8.9 mg) was then added slowly. The reaction mixture was allowed to stir until full conversion was observed by LC-MS. The reaction mixture was allowed to stir until full conversion was observed by LC-MS. The reaction mixture was then directly concentrated. The residue was purified by CombiFlash via a 4-g column of silica gel as the stationary phase with a gradient of DCM to 20% MeOH/DCM (10% B to 100% B) over 20 min., in which product eluted at 100% B. Product was concentrated to provide a white oily residue. LC-MS: calculated [M+H]+ 5558.48 m/z, observed 1152.98 (+5/5, +H₂O) m/z.

### Synthesis of LP49-p

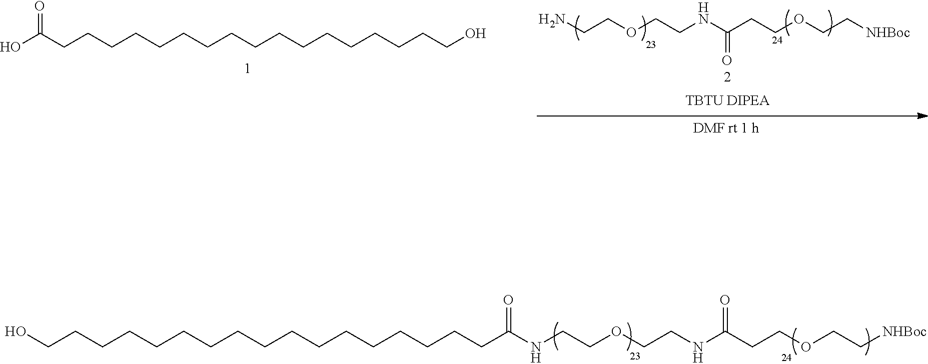

To a solution of compound 1 (31.3 mg), TBTU (33.4 mg), and DIEA (0.023 mL) in DMF was added compound 2 (199 mg) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (10-100%) over 30 min., in which product eluted at 57% B. The product was concentrated under vacuum to provide a white oily residue. LC-MS: calculated [M+H]+ 2583.65 m/z, observed 1311.03 (+2/2, +H₂O)m/z.

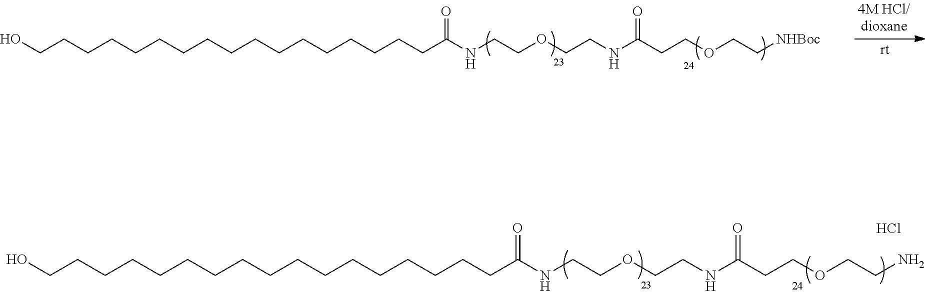

To compound 1 (70 mg) was added 4 M HCl/dioxane (9.9 mg) at room temperature. The reaction was stirred under ambient conditions. Reaction was stirred overnight until full conversion was confirmed via LC-MS. The reaction mixture was azeotroped with PhMe and concentrated under vacuum overnight to provide an oil. LC-MS: calculated [M+H]+ 2483.59 m/z, observed 841.32 (+2/2, +H₂O) m/z.

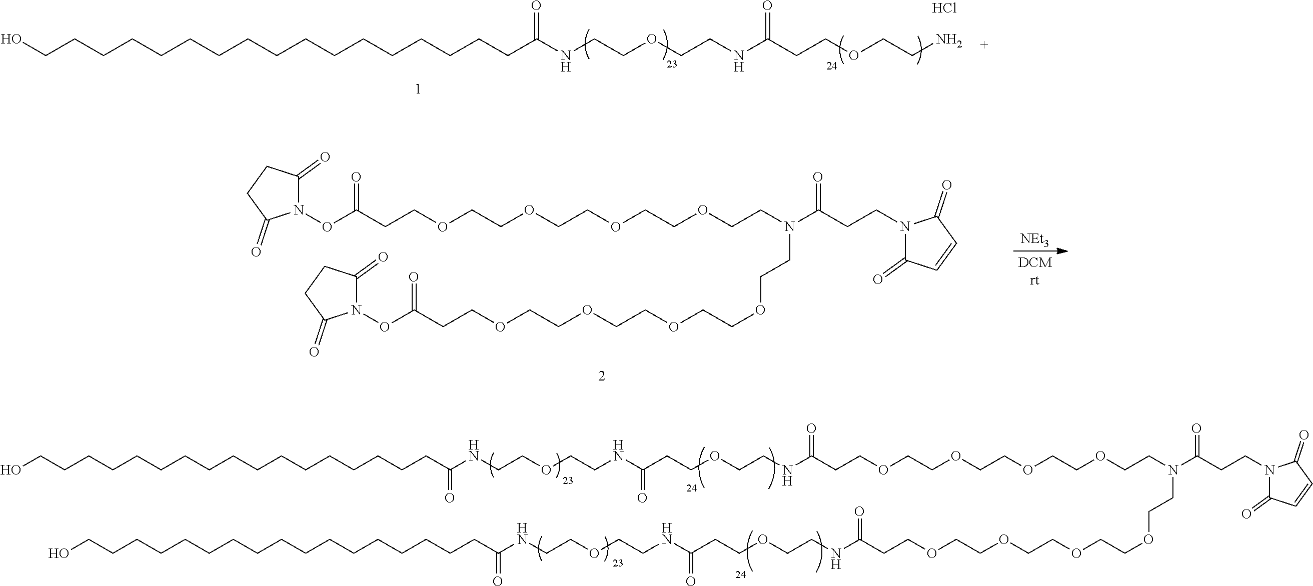

A solution of compound 1 (68.3 mg) and NEt₃ (13.7 mg) in anhydrous DCM under sparging N₂(g) was prepared at room temperature. Compound 2 (11.2 mg) was then added slowly. The reaction mixture was allowed to stir until full conversion was observed by LC-MS. The reaction mixture was allowed to stir until full conversion was observed by LC-MS. The reaction mixture was then directly concentrated. The residue was purified by CombiFlash via a 4-g column of silica gel as the stationary phase with a gradient of DCM to 20% MeOH/DCM (10% B to 100% B) over 20 min., in which product eluted at 100% B. LC-MS: calculated [M+H]+ 5594.97 m/z, observed 1418.68 (+4/4, +H₂O) m/z.

### Synthesis of LP53-p

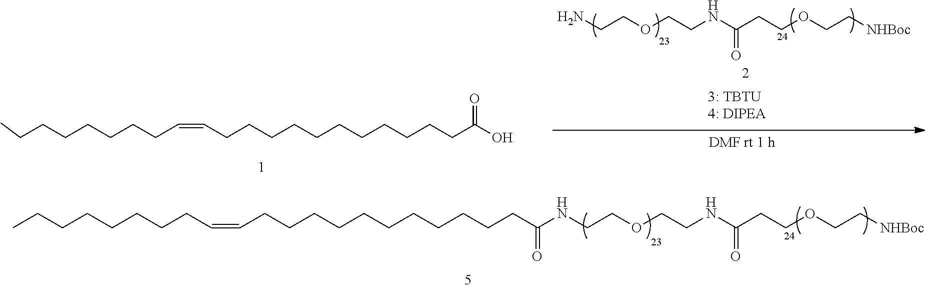

To a solution of compounds 1 (706 mg) and 2 (4.00 g) in DCM was added TBTU (670 mg) and then DIPEA (0.908 mL) under ambient conditions. The reaction was stirred until full conversion was observed by LC-MS. The reaction mixture was then directly concentrated for isolation. The residue was purified by CombiFlash using liquid injection with a gradient of DCM to 20% MeOH in DCM (0-100%) over 40 min. The product was concentrated under vacuum to provide a white oily residue.

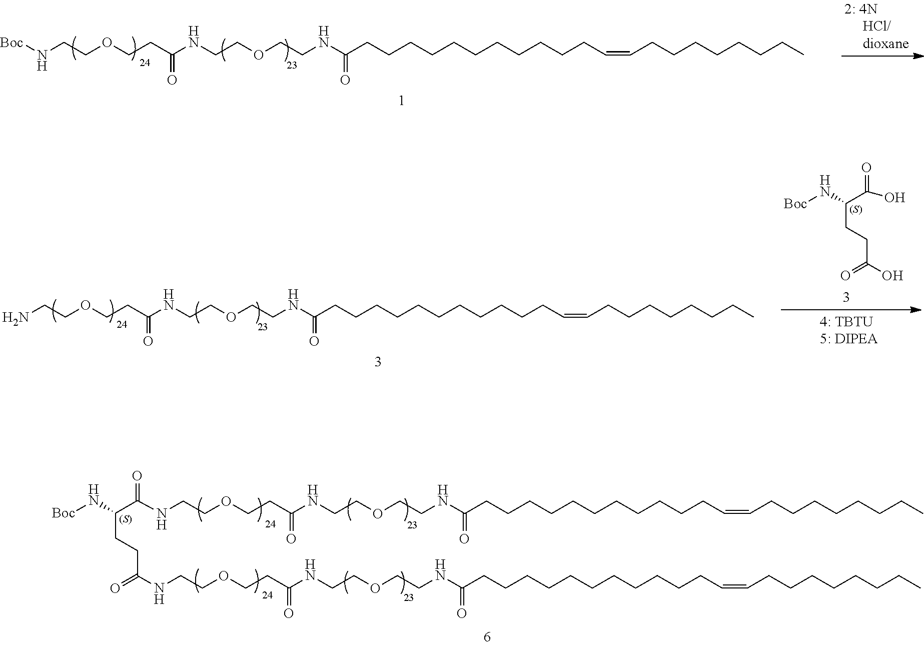

To compound 1 (4.00 g) was added 25 mL 4 M HCl/dioxane at room temperature. The reaction was stirred under ambient conditions. Reaction was stirred 1.5 h until full conversion was confirmed via LC-MS. The reaction was concentrated under vacuum. The residue was dissolved in DCM, then compounds 3 (189 mg), 4 (588 mg) and 5 (0.797 mL) were added. The reaction mixture was stirred at room temperature until full conversion was observed by LC-MS.

The reaction mixture was directly concentrated for isolation. The residue was purified by CombiFlash using silica gel as the stationary phase with a gradient of DCM to 20% McOH in DCM (0-100% B).

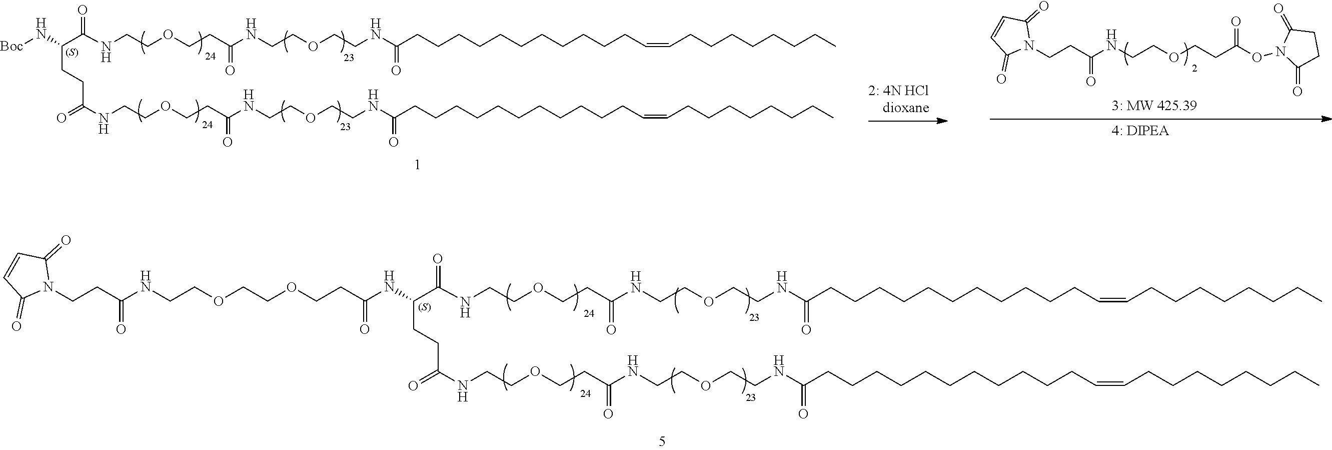

To compound 1 (2.00 g) was added 20 mL 4 M HCl/dioxane at room temperature. The reaction was stirred under ambient conditions for 1.5 h until full conversion was confirmed via LC-MS. The reaction concentrated under vacuum. The residue was dissolved in DCM, then compounds 3 (170 mg) and 4 (148 mg) were added. The reaction mixture was stirred at room temperature until full conversion was observed by TLC.

The product was extracted by a standard work up (1N HCl, sat. NaHCO₃, brine). The residue was purified by CombiFlash using silica gel as the stationary phase with a gradient of DCM to 20% MeOH in DCM (0-100% B).

### Synthesis of LP54-p

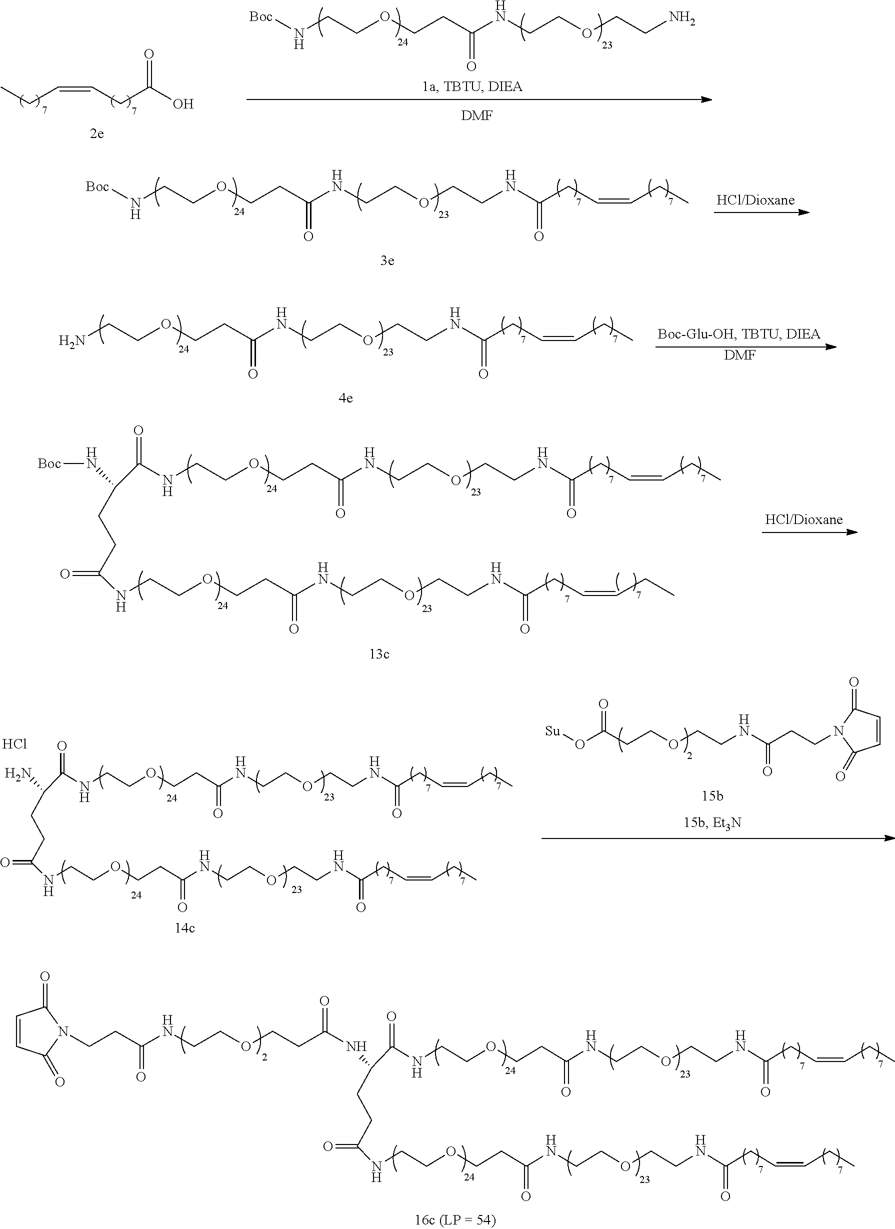

Oleic acid 2e (491 mg, 1.736 mmol) was stirred with Boc-amino-PEG₄₇ derivative 1a, TBTU (670 mg, 2.086 mmol) and DIEA (908 uL, 5.21 mmol) in DMF (50 mL) for 4 h. The solvent was removed in vacuo, toluene was evaporated 3 times from the residue and suspended in CHCl₃ (150 mL). The suspension was washed with H₂O, twice with 2% NaHCO₃, brine, treated with anh. Na₂SO₄, product 3e was concentrated and dried in vacuo. Yield 4.391 g. Calculated MW 2566.24, (M+2×18)/2=1301.12, (M+3×18)/3=873.41 Found: MS (ES, pos): 1301.79 [M+2NH₄]²⁺, 874.08 [M+3NH₄]³⁺.

Compound 3e was converted into amine hydrochloride 4e by treatment with ice-cold 4M HCl/dioxane solution (5 mL) followed by stirring at RT for 1 h. The reaction mixture was concentrated and dried in vacuo, the residual HCl was removed by 2 evaporation of toluene from the product. Amine hydrochloride 4e was stirred with Boc-Glu-OH (197 mg, 0.796 mmol), TBTU (594 mg, 1.85 mmol), and DIEA (1 mL, 5.74 mmol) in DMF:DCM=1:1 (60 mL) for 16 h. The solvent was removed in vacuo, toluene was evaporated 3 times from the residue and suspended in CHCl₃ (300 mL). The suspension was washed with H₂O, twice with 2% NaHCO₃, brine, dried with anh. Na₂SO₄, and product 13c was purified on CombiFlash using the system DCM: 20% MeOH in DCM, gradient 0-100%, 45 min. Yield 2.72 g. Calculated MW 5143.46, (M+3×18)/3=1732.49, (M+4×18)/4=1303.87 Found: MS (ES, pos): 1733.46 [M+3NH₄]³⁺, 1304.55 [M+4NH₄]⁴⁺.

13c (2.72 g, 0.529 mmol) was stirred in 4M HCl/dioxane solution (30 mL) for 1 h, the solvent was removed in vacuo, toluene was evaporated 2 times from the residue and dry hydrochloride salt 14c was stirred with NHS-ester 15b (212 mg, 0.5 mmol) and Et3N in DCM (45 mL) for 16 h. The reaction mixture was diluted 3 times with CHCl₃, washed with H₂O, and brine, dried (Na₂SO₄), concentrated and product 16c was purified on CombiFlash using the system DCM: 20% MeOH in DCM, gradient 0-100%, 55 min. Yield 440 mg. Calculated MW 5353.65, (M+3×18)/3=1802.55, (M+4×18)/4=1356.41 Found: MS (ES, pos): 1803.19 [M+3NH₄]³⁺, 1357.24 [M+4NH₄]⁴⁺.

### Synthesis of LP55-p

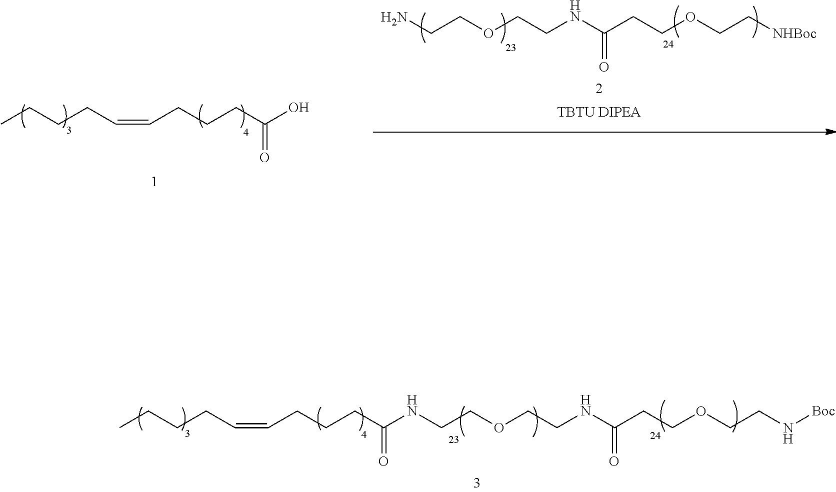

To a solution of compounds 1 (297 mg) and 2 (2.00 g) in DCM was added TBTU (307 mg) and then DIPEA (0.454 mL) at room temperature. The reaction mixture was stirred until full conversion was observed by LC-MS. The product was extracted by standard work up (1N HCl, sat. NaHCO₃, brine wash) and dried over Na₂SO₄. The crude compound 3 was used directly in the next step.

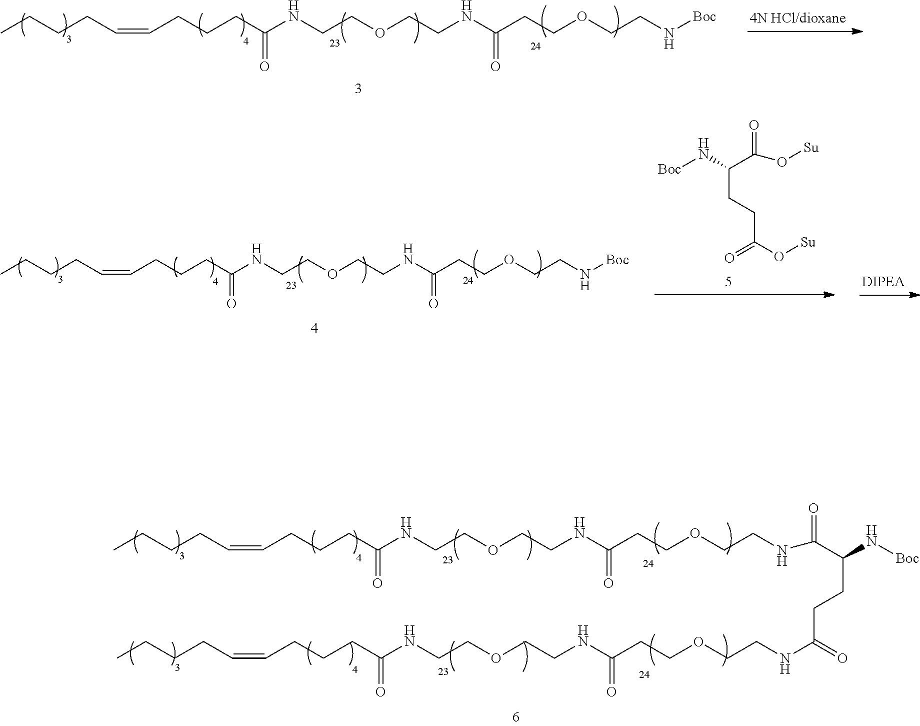

To compound 3 (2.00 g) was added 20 mL 4 M HCl/dioxane at room temperature. The reaction mixture was stirred at room temperature for 1.5 h until full conversion was confirmed via LC-MS. The reaction mixture was concentrated under vacuum. The residue was dissolved in DCM, then DIPEA (0.0403 mL) was added. followed by slow addition of compound 5 (160 mg in DCM) using a syringe pump (in 2-3 hours). The reaction mixture was stirred at room temperature until full conversion was observed by TLC.

The product was extracted using a standard work up (1N HCl, sat. NaHCO₃, brine). The residue was purified by CombiFlash® using silica gel as the stationary phase with a gradient of 0-20% MeOH in DCM (0-100% B) to afford compound 6.

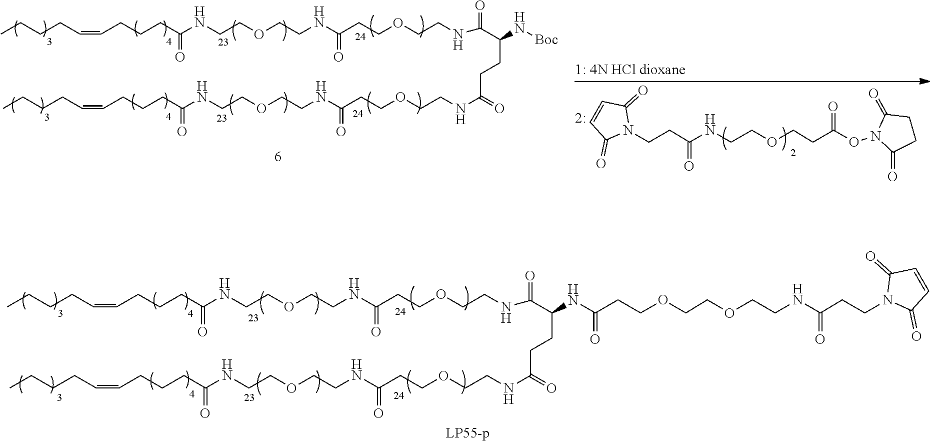

To compound 6 (1.22 g) was added 10 mL 4 M HCl/dioxane at room temperature. The reaction mixture was stirred at room temperature for 1.5 h until full conversion was confirmed via LC-MS. The reaction mixture was concentrated under vacuum. The residue was dissolved in DCM, then compound 7 (105 mg) and DIPEA (148 mg) were added. The reaction mixture was stirred at room temperature until full conversion was observed by TLC.

The product LP55-p was extracted using a standard workup (1N HCl, sat. NaHCO₃, brine). The residue was purified by CombiFlash® using silica gel as the stationary phase with a gradient of 0-20% MeOH in DCM (0-100% B).

### Synthesis of LP56-p

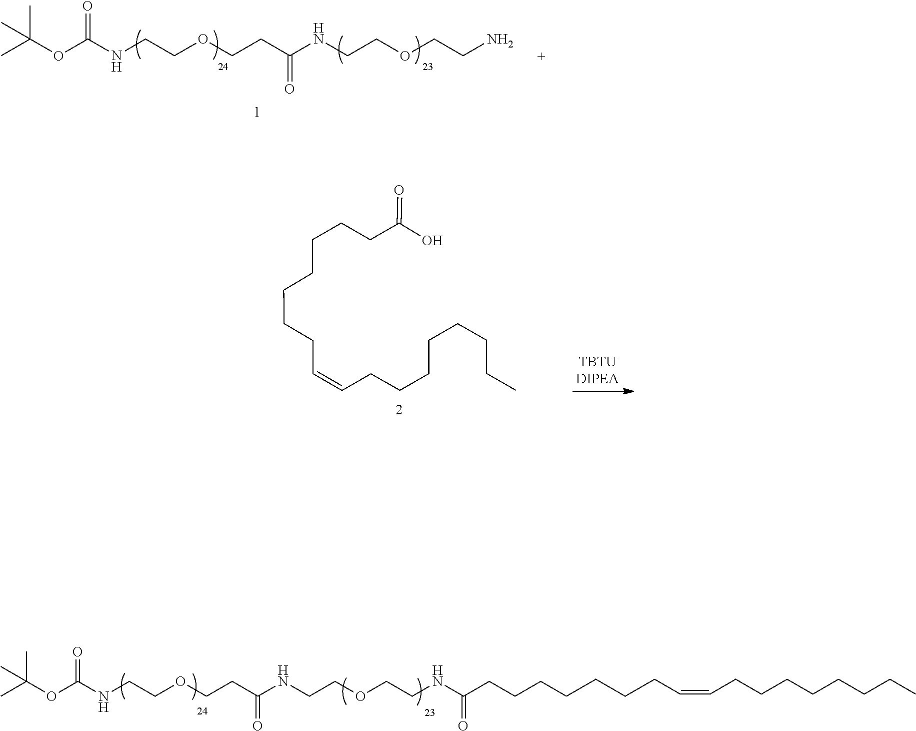

To a solution of compound 1 (150 mg, 0.0652 mmol, 1.0 equiv.), compound 2 (20 mg, 0.0717 mmol, 1.1 equiv.) and diisopropylethylamine (0.034 mL, 0.195 mmol, 3.0 equiv.) in anhydrous DMF (3 mL) was added TBTU (25.1 mg, 0.0782 mmol, 1.2 equiv.) at room temperature. The reaction was kept at room temperature for 2 hrs. The reaction mixture was concentrated. The product was purified by CombiFlash and was eluted with 12-18% methanol in dichloromethane. LC-MS: calculated [M+2H]+/2 1283.32, found 1283.87.

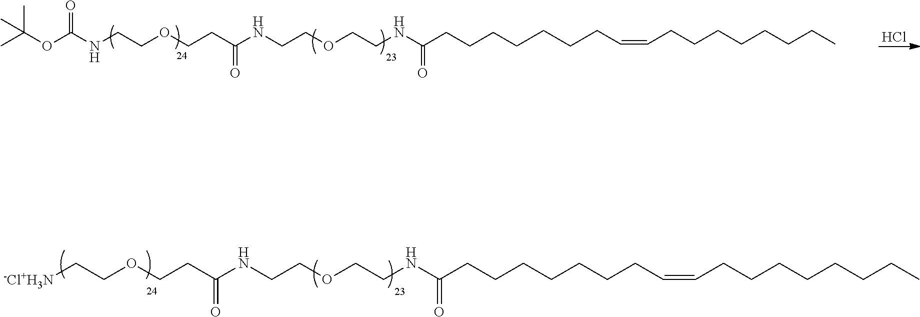

To solid of compound 1 (82 mg, 0.0320 mmol, 1.0 equiv.) was added HCl solution in dioxane (0.4 mL, 1.597 mmol, 50 equiv.) at room temperature. The reaction was kept at room temperature for 30 min and the solvent was concentrated. The product was used directly without further purification. LC-MS: calculated [M+2H]+/2 1233.29, found 1233.69.

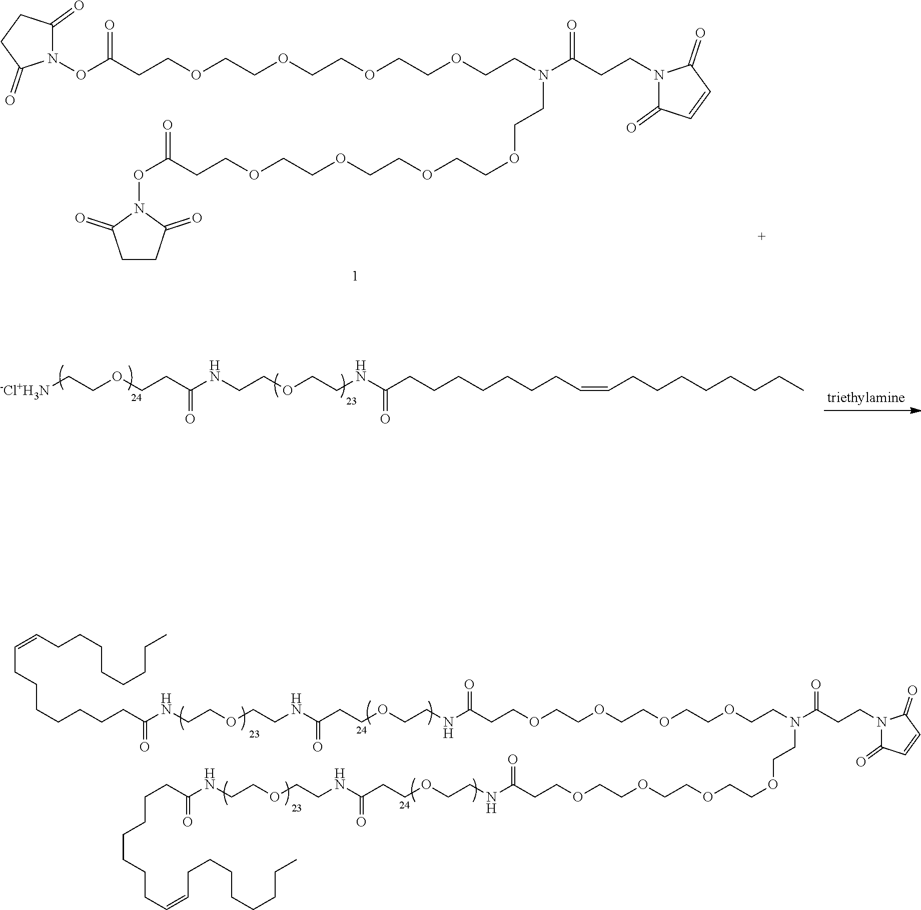

To a solution of compound 1 (13 mg, 0.0151 mmol, 1.0 equiv.) and compound 2 (77.7 mg, 0.0310 mmol, 2.05 equiv.) in anhydrous DCM (2 mL) was added triethylamine (0.011 mL, 0.0757 mmol, 5.0 equiv.) at room temperature. The reaction was kept at room temperature for 1 hr and the solvent was concentrated. The product was separated by CombiFlash and was eluted with 12-18% methanol in dicholoromethane. LC-MS: calculated [M+5H]+/5 1112.49, found 1112.34, calculated [M+6H]+/6 927.24, found 927.97.

### Synthesis of LP57-p

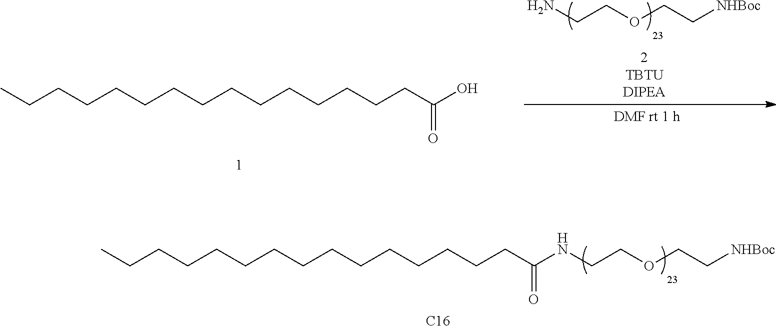

To a solution of compound 1 (787 mg), TBTU (985 mg), and DIEA (662 mg) in DMF was added compound 2 (3.06 g) under ambient conditions. The reaction was stirred overnight until full conversion was observed by LC-MS. The reaction mixture was then washed with NaHCO₃ and extracted with 20% trifluoroethanol/DCM. The residue was purified by CombiFlash using an 80-g column of silica gel as the stationary phase with a gradient of DCM to 20% MeOH in DCM (0-100%) over 45 min., in which product eluted at 28% B. The product was concentrated under vacuum to provide a white oily residue. LC-MS: calculated [M+H]+ 1411.95 m/z, observed 724.80 (+2/2, +H₂O) m/z.

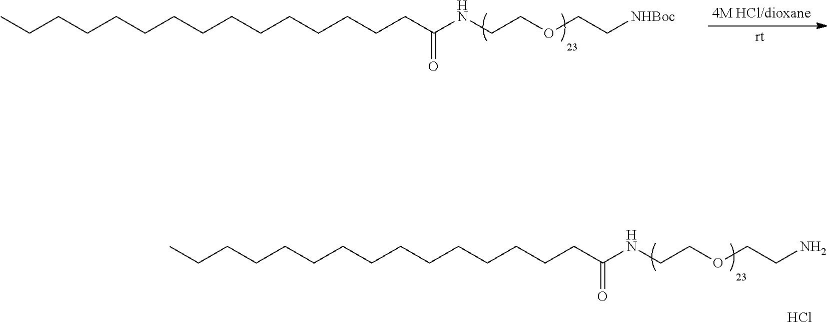

To compound 1 (1.27 g) was added 4 M HCl/dioxane (329 mg) at room temperature. The reaction was stirred under ambient conditions until full conversion was confirmed via LC-MS. The reaction mixture was azeotroped with PhMe/MeOH and concentrated under high-vacuum overnight to provide a white solid. LC-MS: calculated [M+H]+1311.90 m/z observed 657.59 (+2/2) m/z.

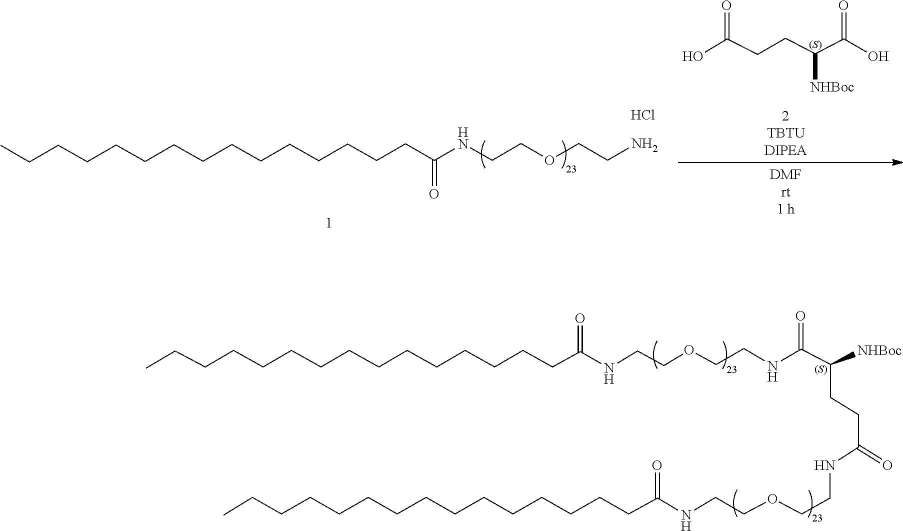

To a solution of compound 1 (1.22 g), TBTU (348 mg), and DIEA (0.3825 mL) in DMF was added compound 2 (109 mg) under ambient conditions. The reaction was stirred until full conversion was observed by LC-MS. The reaction mixture was then washed with NaHCO₃, extracted with 20% 2, 2,2-trifluoroethanol (TFE)/DCM, washed with NH₄Cl soln., dried over Na₂SO₄, filtered, and concentrated for isolation. The residue was purified by CombiFlash using silica gel as the stationary phase with a gradient of DCM to 20% MeOH in DCM (0-100%) over 30 min., in which product eluted at 51% B. Clean and impure fractions were collected and concentrated. The impure fraction was reisolated via DCM to 20% MeOH/DCM (0-100% B), in which product eluted at 54% B and was collected and concentrated in pure fractions. Concentration via vacuum provided a white oily residue. LC-MS: calculated [M+H]+ 2833.89 m/z, observed 727.56 (+4/4, +H₂O) m/z.

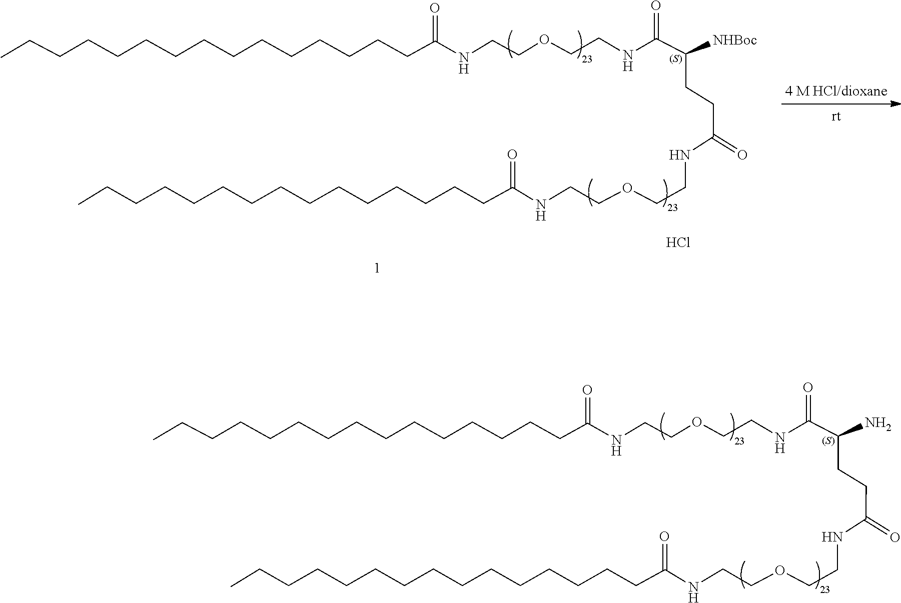

To compound 1 (130 mg) was added 4 M HCl/dioxane (16.7 mg) at room temperature. The reaction was stirred under ambient conditions until full conversion was confirmed via LC-MS. The reaction mixture was azeotroped with PhMe/MeOH and concentrated under high-vacuum overnight to provide a white solid. LC-MS: calculated [M+H]+ 2769.81 m/z, observed 694.07 (+HCl, +4/4) m/z.

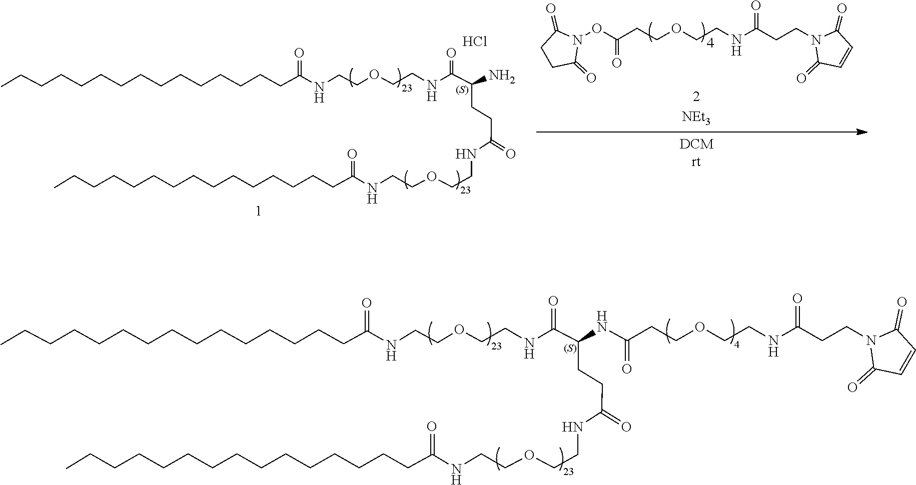

A solution of compound 1 (127 mg) and NEt₃ (0.026 mL) in anh. DCM under sparging N₂(g) was prepared at room temperature. Compound 2 (24.8 mg) was then added slowly. The reaction mixture was allowed to stir until full conversion was observed by LC-MS. The reaction mixture was then directly concentrated. The residue was purified by CombiFlash via a 12-g column of silica gel as the stationary phase with a gradient of DCM to 20% MeOH/DCM (0% B to 100% B) over 20 min., in which product eluted at 100% B. LC-MS: calculated [M+H]+ 3132.00 m/z, observed 1584.89 (+3/3, +H₂O) m/z.

### Synthesis of LP58-p

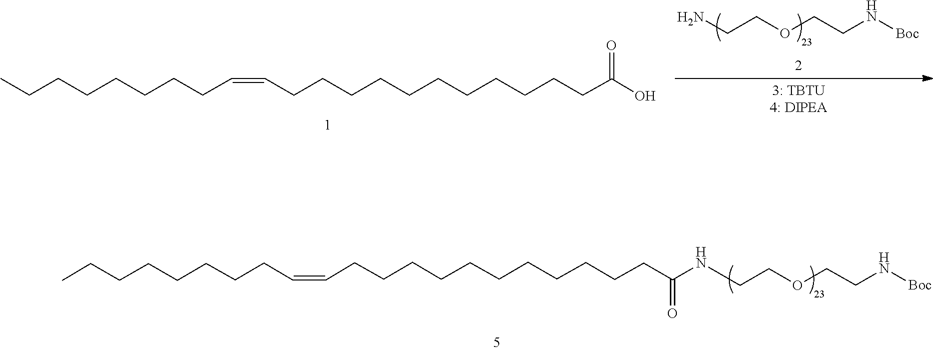

To a solution of compounds 1 (606 mg) and 2 (2.00 g) in DCM was added TBTU (657 mg) and then DIPEA (0.891 mL) under ambient conditions. The reaction was stirred until full conversion was observed by LC-MS. The reaction mixture was then directly concentrated for isolation. The residue was purified by CombiFlash using liquid injection with a gradient of DCM to 20% MeOH in DCM (0-100%) over 40 min. The product was concentrated under vacuum to provide a white oily residue.

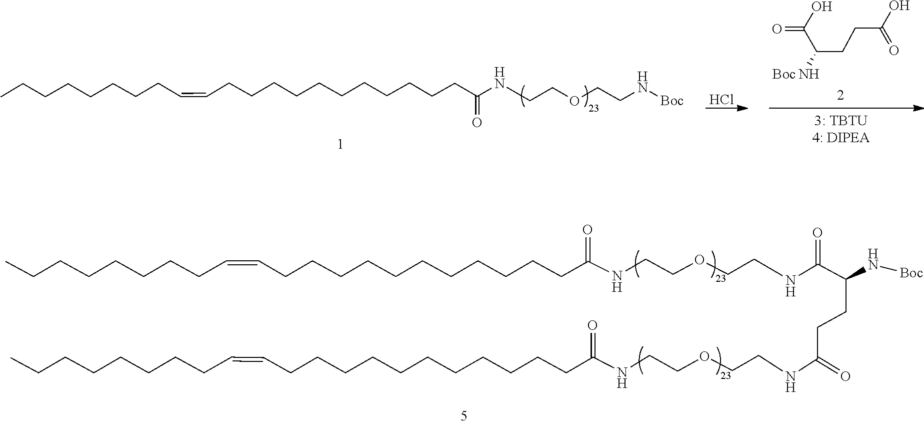

To compound 1 (2.20 g) was added 5 mL 4 M HCl/dioxane at room temperature. The reaction was stirred under ambient conditions for 1.5 h until full conversion was confirmed via LC-MS. The reaction concentrated under vacuum. The residue was dissolved in DCM, then compounds 2 (171 mg), 3 (567 mg) and 4 (0.770 mL) were added. The reaction mixture was stirred at room temperature until full conversion was observed by TLC.

The product was extracted using a standard work up (1N HCl, sat. NaHCO₃, brine). The residue purified by CombiFlash using silica gel as the stationary phase with a gradient of DCM to 20% MeOH in DCM (0-100% B).

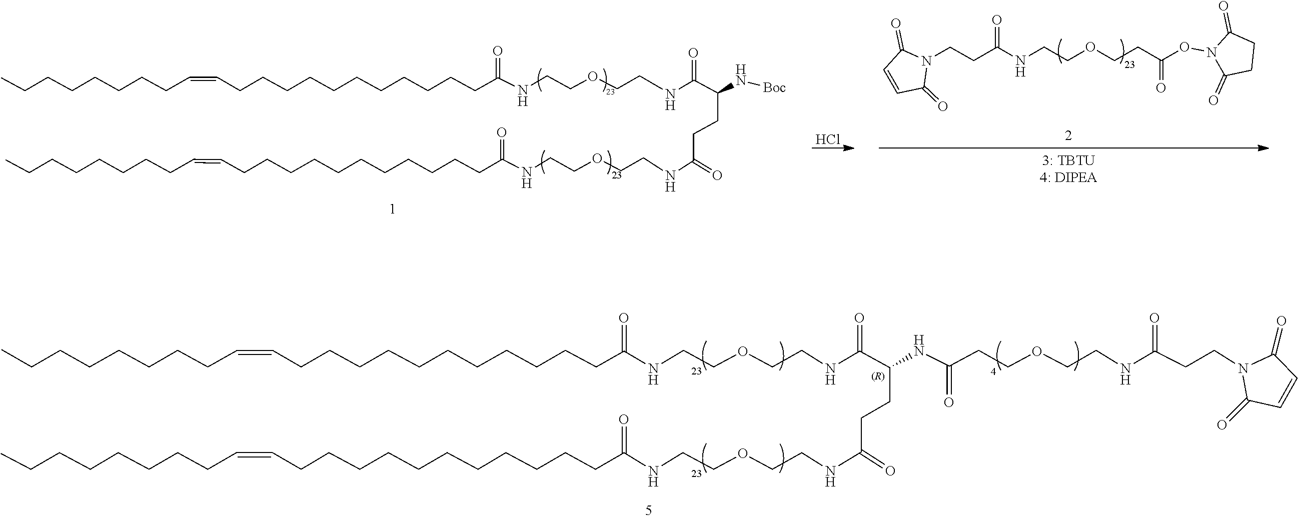

To compound 1 (1.34 g) was added 10 mL 4 M HCl/dioxane at room temperature. The reaction was stirred under ambient conditions for 1.5 h until full conversion was confirmed via LC-MS. The reaction concentrated under vacuum. The residue was dissolved in DCM, then compounds 3 (172 mg) and 4 (0.234 mL) were added. The reaction mixture was stirred at room temperature until full conversion was observed by TLC.

The product was extracted using a standard work up (1N HCl, sat. NaHCO₃, brine). The residue was purified by CombiFlash using silica gel as the stationary phase with a gradient of DCM to 20% MeOH in DCM (0-100% B).

### Synthesis of LP59-p

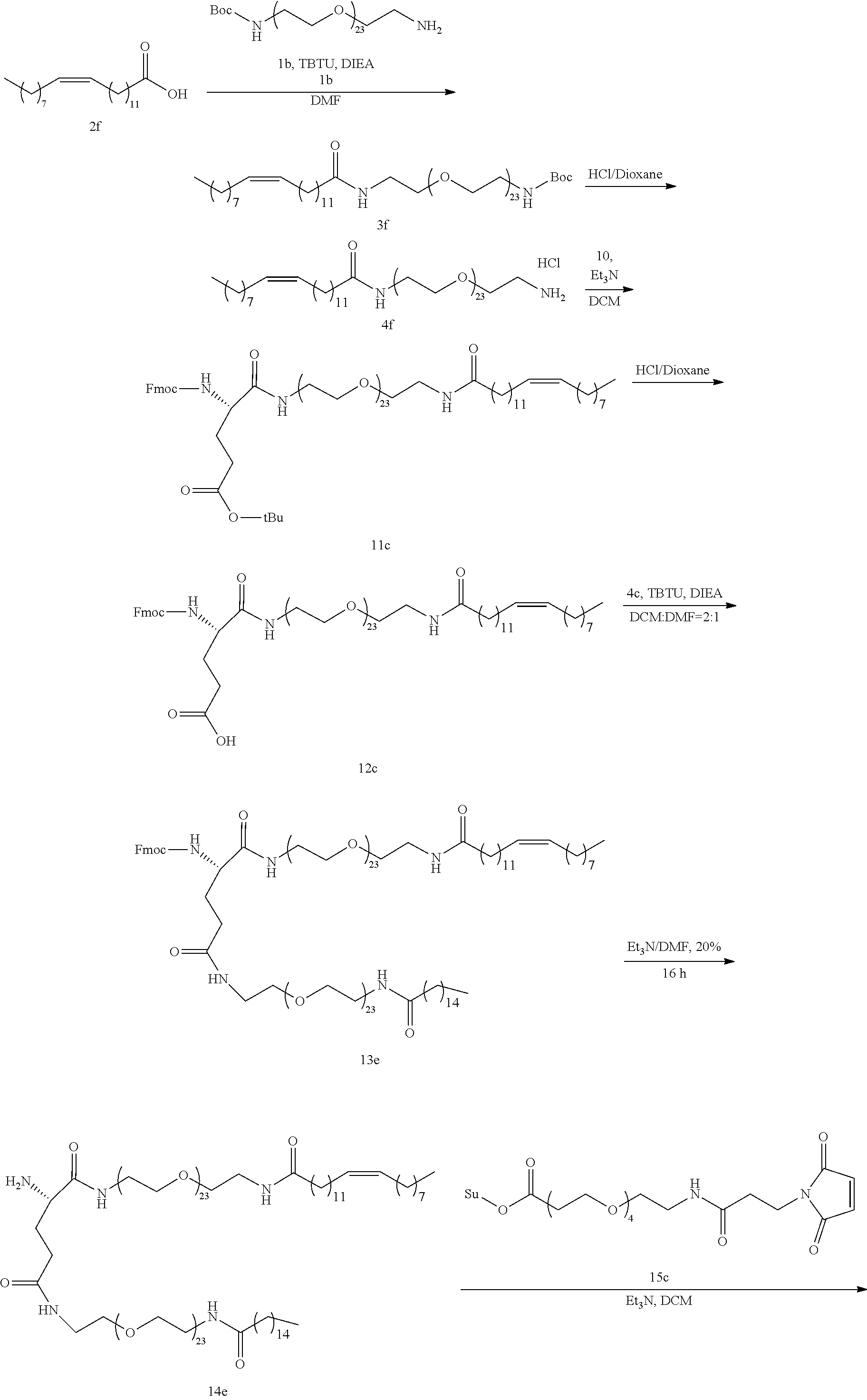

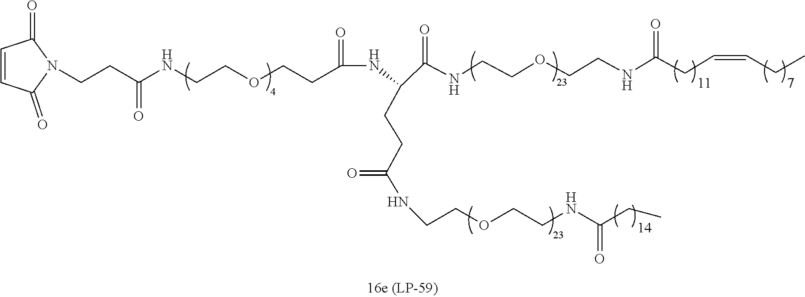

Erucic acid 2f (587 mg, 1.736 mmol) was stirred with Boc-aminopeg47 derivative 1b, TBTU (670 mg, 2.086 mmol) and DIEA (908 uL, 5.21 mmol) in DMF (50 mL) for 4 h. The solvent was removed in vacuo, toluene was evaporated 3 times from the residue and suspended in CHCl₃ (150 mL). The suspension was washed with H₂O, twice with 2% NaHCO₃, brine, treated with anh. Na₂SO₄, product 3f was concentrated and dried in vacuo. Yield 4.391 g. Calculated MW 1494.00, M+18=1512.00, (M+2×18)/2=765.00. Found: MS (ES, pos): 1512.53 [M+NH₄]⁺, 765.72 [M+2NH₄]²⁺.

The Boc protecting-group was removed with 4M solution of HCl in dioxane to obtain hydrochloride salt 4f (1.192 g, 0.834 mmol), which was directly used in the next step without purification. Pentafluorophenyl ester 10 (493 mg, 0.834 mmol) and Et3N (290 uL, 2.084 mmol) in DCM (30 mL) were mixed with hydrochloride salt 4f After 2 h of stirring the reaction mixture was diluted with CHCl₃ (150 mL), washed with H₂O, aqueous 3% NaHCO₃, and brine. The dried product 11c 1.539 g was directly used in the following step.

11c (1.539 g, 0.834 mmol) was stirred in 4M HCl/Dioxane solution (20 mL) for 4h. The solvent was removed in vacuo, toluene was evaporated 2 times from the residue to obtain dry deprotected acid 12c (1.52 g, 0.827 mmol). This acid was stirred with amine hydrochloride 4c (1.114 g, 0.827 mmol, synthesized as shown in synthesis for LP39, above), TBTU (318.6 mg, 0.992 mmol), and DIEA (532 uL, 3.05 mmol) in a mixture of DCM:DMF=1:2 (30 mL) for 16 h. The solvent was removed in vacuo, the residual DMF was removed with 3 additional evaporation of toluene. The residue was suspended in CHCl₃ (150 mL), washed with H₂O, twice with 3% NaHCO₃, and brine. Following drying with Na₂SO₄ the product 13e was concentrated and purified on CombiFlash using the system DCM: 20% MeOH in DCM, gradient 0-100%, 55 min. Yield 1.429 g. Calculated MW 3038.96, (M+2×18)/2=1537.48, (M+3×18)/3=1030.99 Found: MS (ES, pos): 1537.97 [M+2NH₄]²⁺, 1031.66 [M+3NH₄]³⁺.

The product 13e was Fmoc-deprotected as described in the procedure for LP39, above. The product 14e was dried and reacted with NHS-ester 15c as described in the procedure for LP39, above. The product 16e was isolated using CombiFlash purification. Calculated MW 3215.13, (M+2×18)/2=1625.57, (M+3×18)/4=1089.71. Found: MS (ES, pos): 1626.30 [M+2NH₄]²⁺, 1090.58 [M+3NH₄]³⁺.

### Synthesis of LP60-p

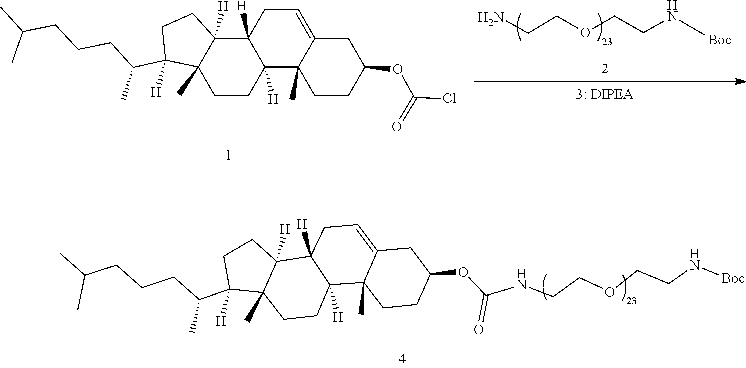

To a solution of compounds 1 (278 mg) and 2 (1.00 g) in DCM was added compound 3 (0.223 mL.) The reaction was stirred until full conversion of 2 was observed by TLC. The product was extracted using a standard work up (1N HCl, sat. NaHCO₃, brine) and dried over Na₂SO₄. The crude product was used directly in the next step.

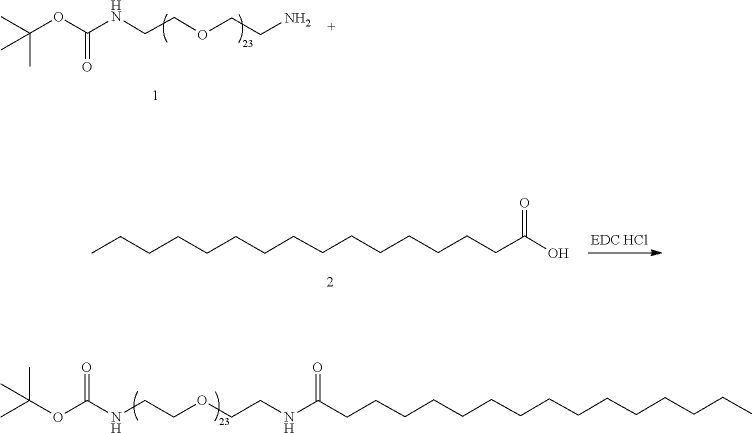

To a solution of compound 1 (2500 mg, 2.130 mmol, 1.0 equiv.) and compound 2 (655 mg, 2.556 mmol, 1.2 equiv.) in anhydrous DCM (10 mL) was added EDC HCl (630 mg, 3.195 mmol, 1.5 equiv.) at room temperature. The reaction was kept at room temperature overnight. The reaction mixture was concentrated. The product was purified by CombiFlash and was eluted with 12-20% methanol in dichloromethane. LC-MS: calculated [M+H]+ 1411.95. found 1413.64.

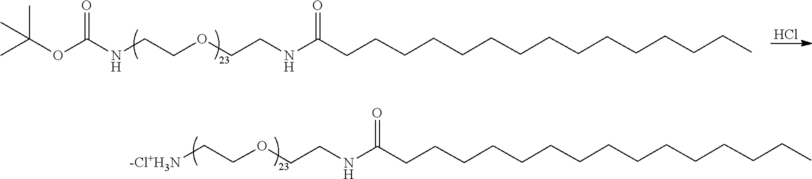

To a solid of compound 1 (2100 mg, 1.487 mmol, 1.0 equiv.) was added HCl solution in dioxane (7.438 mL, 29.75 mmol, 20 equiv.) at room temperature. The reaction was kept at room temperature for 1 hr and the solvent was concentrated. The product was used directly without further purification. LC-MS: calculated [M+H]+ 1311.90, found 1312.95.

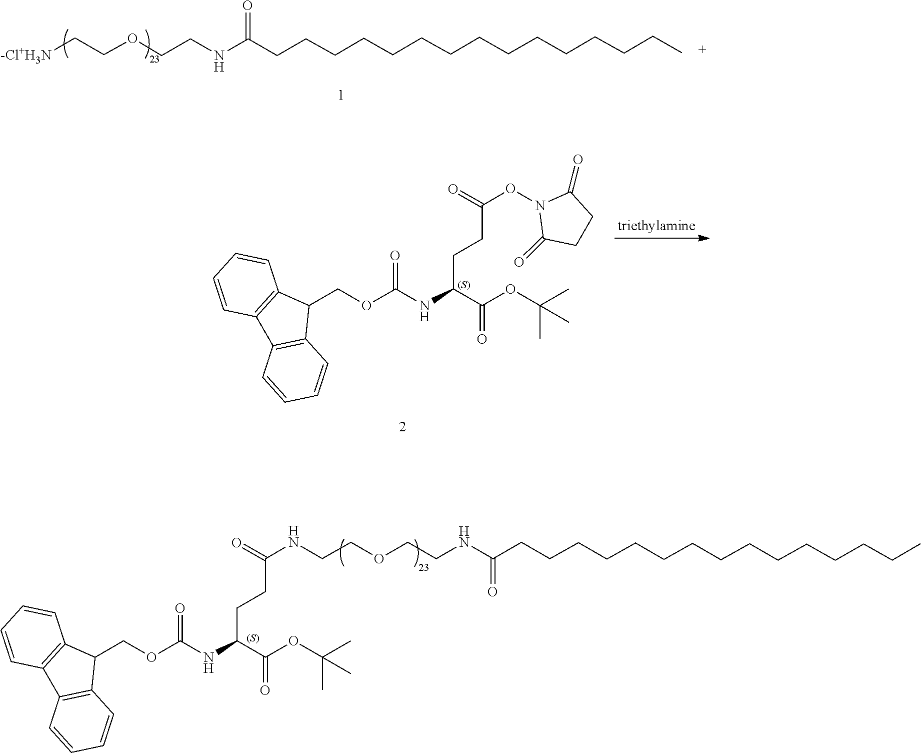

To a solution of compound 1 (1210 mg, 0.897 mmol, 1.0 equiv.) and compound 2 (539 mg, 1.032 mmol, 1.15 equiv.) in anhydrous DCM (10 mL) was added triethylamine (0.381 mL, 2.692 mmol, 3.0 equiv.) at room temperature. The reaction was kept at room temperature for 2 hrs. The organic phase was washed with saturated NH₄Cl and saturated NaHCO₃ aqueous solution. The organic phased was dried over Na₂SO₄ and concentrated. The product was separated by CombiFlash and was eluted with 12-20% methanol in dicholoromethane. LC-MS: calculated [M+H]+ 1719.07, found 1719.42.

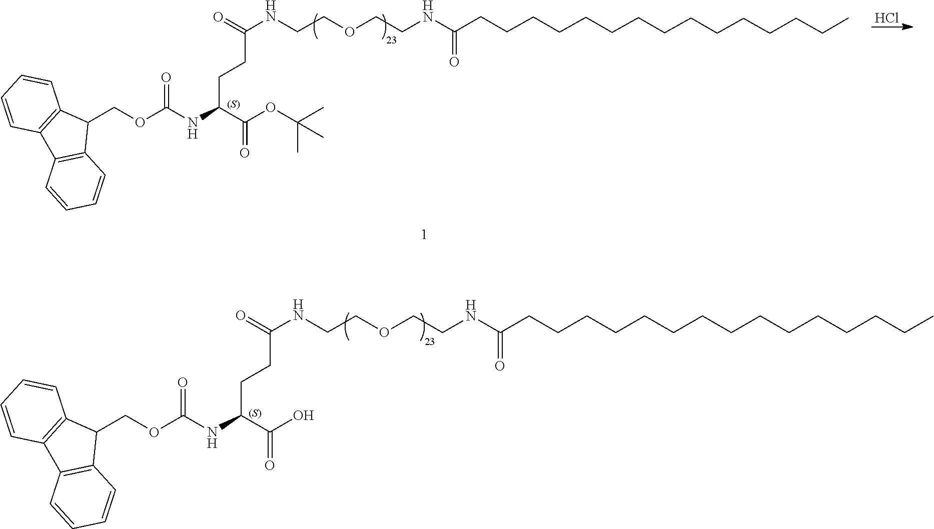

To compound 1 (1100 mg, 0.639 mmol, 1.0 equiv.) was added 4M HCl in dioxane (3.199 mL, 12.796 mmol, 20 equiv.) at room temperature. The reaction was kept at room temperature for 8 hrs. The reaction mixture was concentrated. The product was used directly without further purification. LC-MS: [M+H]+ calculated 1663.01, found 1664.00.

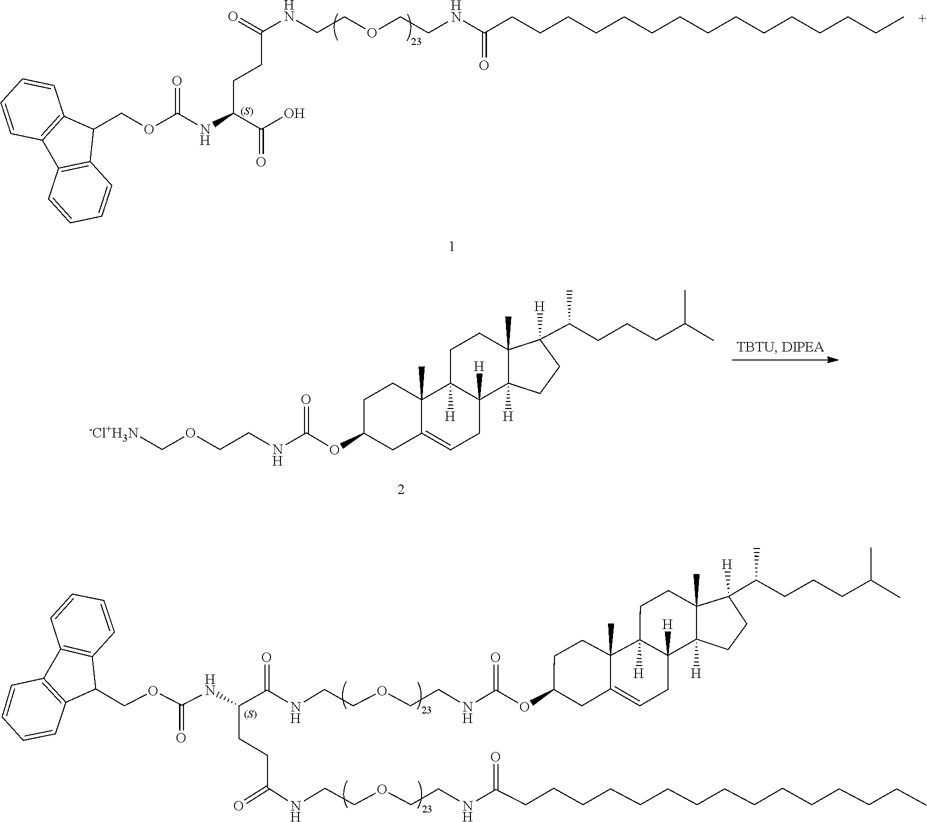

To a solution of compound 1 (1060 mg, 0.637 mmol, 1.0 equiv.), compound 2 (970 mg, 0.637 mmol, 1.00 equiv.) and diisopropylethylamine (0.444 mL, 2.549 mmol, 4.0 equiv.) in DMF (10 mL) was added TBTU (245 mg, 0.764 mmol, 1.2 equiv.) at room temperature. The reaction was kept at room temperature for 2 hrs. The reaction mixture was concentrated. The residue was washed with saturated ammonium choloride and sodium bicarbonate aqueous solution. The product was purified by CombiFlash and was eluted with 10-20% methanol in dichloromethane. LC-MS: [M+2H]/2 calculated 1565.50, found 1567.13.

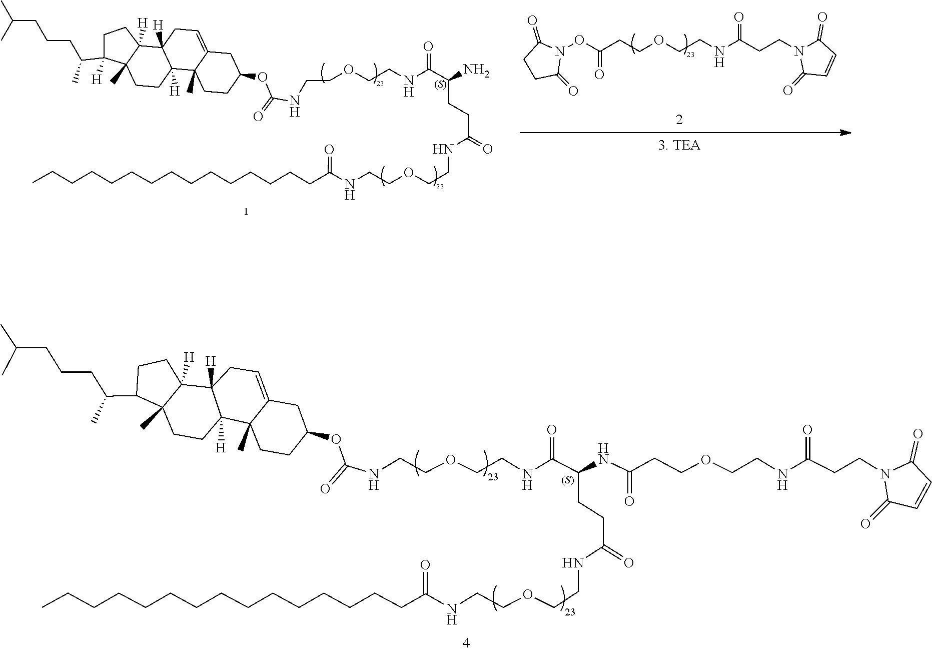

To a solution of compound 1 (585 mg) in 6 mL DCM was added compounds 2 (124 mg) and 3 (0.085 mL) under ambient conditions. The reaction was stirred overnight. The product was extracted using a standard work up (1N HCl, sat. NaHCO₃, brine) and dried over Na₂SO₄. The product was further purified with column chromatography.

### Synthesis of LP61-p

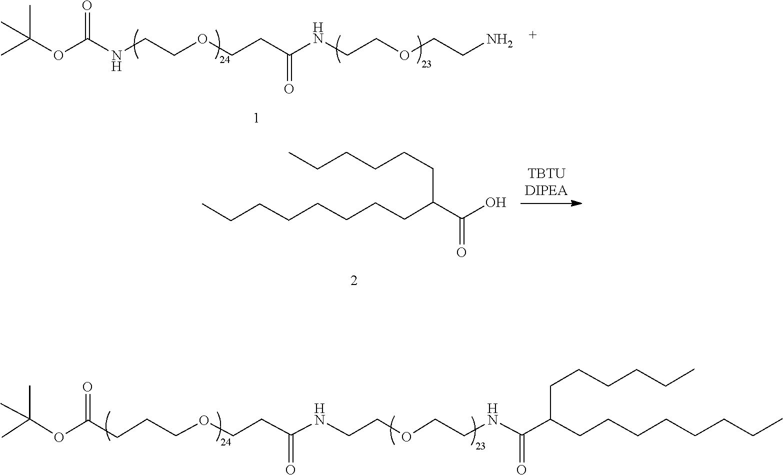

To a solution of compound 1 (124 mg, 0.0539 mmol, 1.0 equiv.), compound 2 (19 5 mg, 0.0646 mmol, 1.2 equiv.) and diisopropylethylamine (0.028 mL, 0.161 mmol, 3.0 equiv.) in anhydrous DMF (2 mL) was added TBTU (20.8 mg, 0.0646 mmol, 1.2 equiv.) at room temperature. The reaction was kept at room temperature for 1 hr. The reaction mixture was quenched with saturated sodium bicarbonate aqueous solution. The aqueous phase was extracted with DCM (3×10 mL), and the organic phase was combined, dried over Na₂SO₄, and concentrated. The product was purified by CombiFlash and was eluted with 10-12% methanol in dichloromethane. LC-MS: calculated [M+2H]+/2 1270.31, found 1269.15.

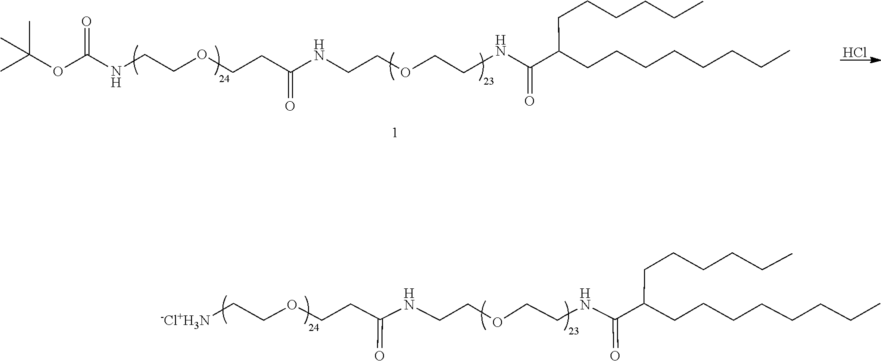

To compound 1 (56 mg, 0.0220 mmol, 1.0 equiv.) was added 4M HCl in dioxane (0.276 mL, 1.102 mmol, 50 equiv.) at room temperature. The reaction was kept at room temperature for 1 hr. The reaction mixture was concentrated. The product was used directly without further purification. LC-MS: [M+2H]/2 calculated 1220.28, found 1221.63.

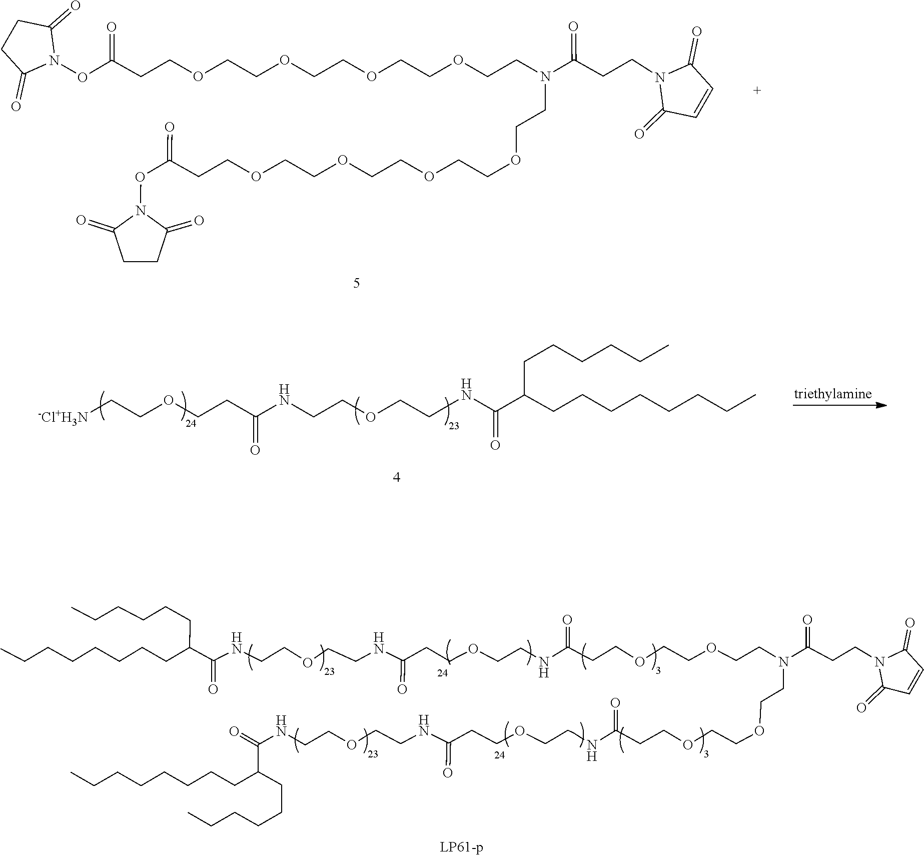

To a solution of compound 1 (10 mg, 0.0116 mmol, 1.0 equiv.) and compound 2 (59.1 mg, 0.0239 mmol, 2.05 equiv.) in anhydrous DCM (2 mL) was added triethylamine (0.008 mL, 0.0931 mmol, 5.0 equiv.) at room temperature. The reaction was kept at room temperature for 4 hrs and the solvent was concentrated. The product was separated by CombiFlash and was eluted with 12-15% methanol in dicholoromethane. LC-MS: calculated [M+6H]+/6 918.57, found 919.69.

### Synthesis of LP62-p

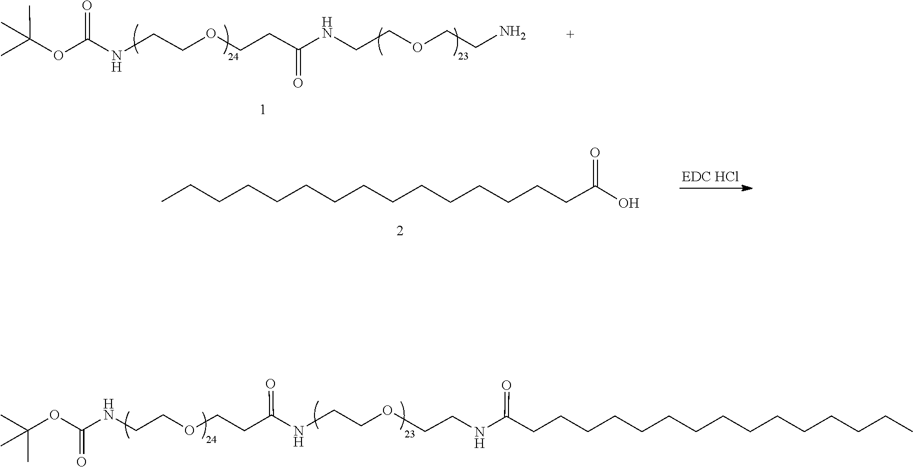

To a solution of compound 1 (1500 mg, 0.6517 mmol, 1.0 equiv.) and compound 2 (200 mg, 0.782 mmol, 1.2 equiv.) in anhydrous DCM (10 mL) was added EDC HCl (192 mg, 0.997 mmol, 1.5 equiv.) at room temperature. The reaction was kept at room temperature overnight. The reaction mixture was concentrated. The product was purified by CombiFlash and was eluted with 12-20% methanol in dichloromethane. LC-MS: calculated [M+2H]+/2 1270.31, found 1271.43.

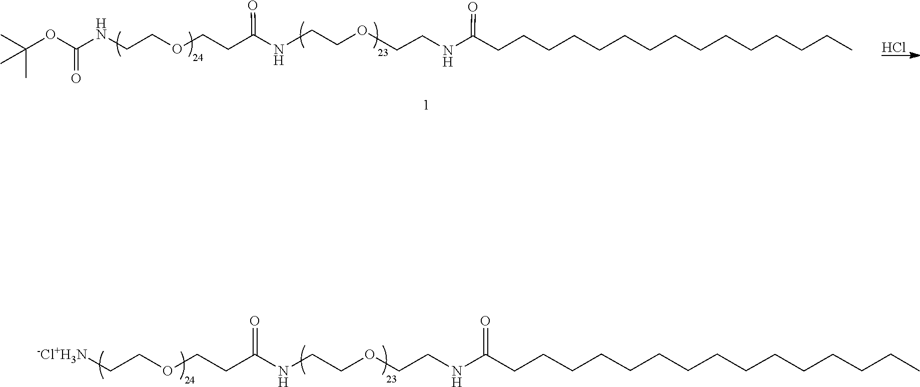

To compound 1 (1300 mg, 0.511 mmol, 1.0 equiv.) was added 4M HCl in dioxane (6.397 mL, 25.588 mmol, 50 equiv.) at room temperature. The reaction was kept at room temperature for 1 hr. The reaction mixture was concentrated. The product was used directly without further purification. LC-MS: [M+2H]/2 calculated 1220.28, found 1221.87.

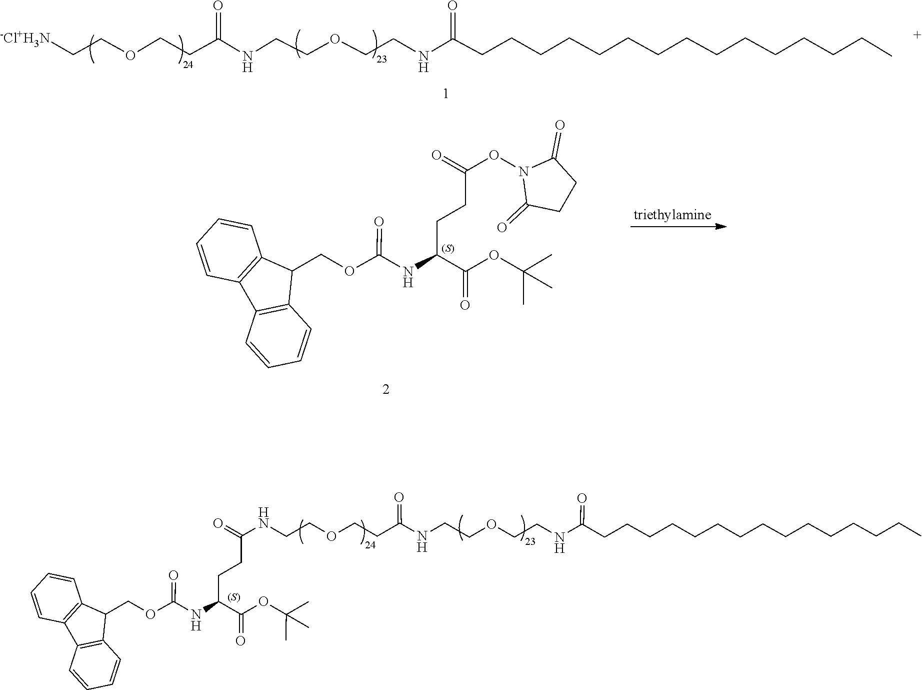

To a solution of compound 1 (1350 mg, 0.0 mmol, 1.0 equiv.) and compound 2 (327 mg, 0.626 mmol, 1.15 equiv.) in anhydrous DCM (10 mL) was added triethylamine (0.231 mL, 1.625 mmol, 3.0 equiv.) at room temperature. The reaction was kept at room temperature overnight and the solvent was concentrated. The product was separated by CombiFlash and was eluted with 12-20% methanol in dicholoromethane. LC-MS: calculated [M+3H]/3 949.58, found 950.77.

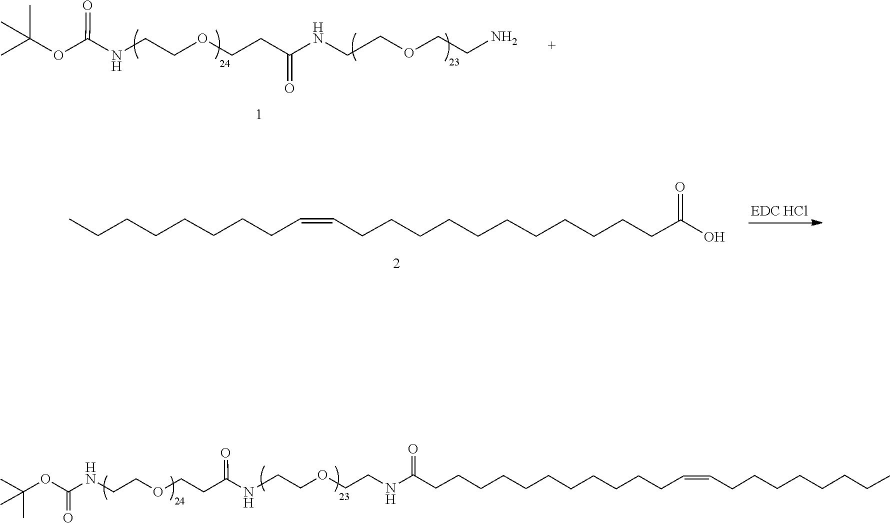

To a solution of compound 1 (1500 mg, 0.6517 mmol, 1.0 equiv.) and compound 2 (265 mg, 0.782 mmol, 1.2 equiv.) in anhydrous DCM (10 mL) was added EDC HCl (192 mg, 0.997 mmol, 1.5 equiv.) at room temperature. The reaction was kept at room temperature for 3 hrs. The reaction mixture was concentrated. The product was purified by CombiFlash and was eluted with 12-20% methanol in dichloromethane. LC-MS: calculated [M+2H]+/2 1311.35, found 1311.87.

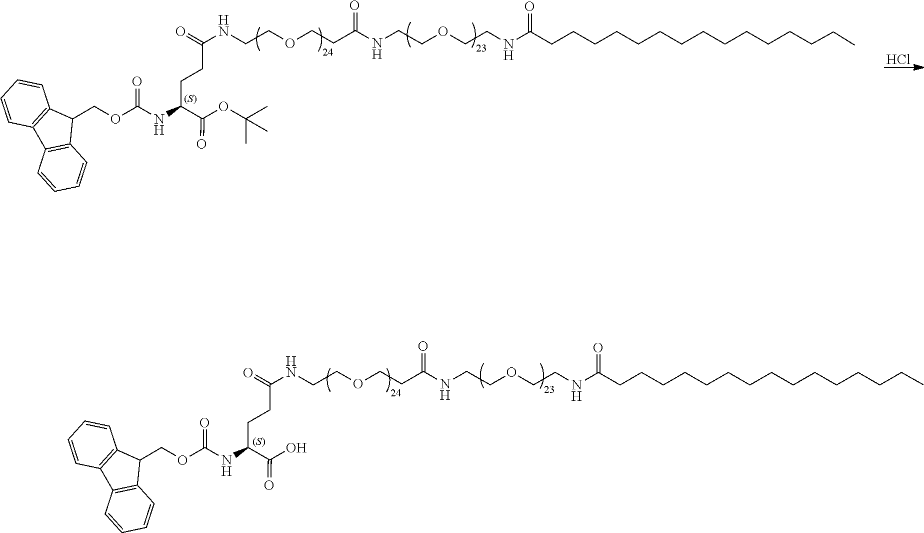

To compound 1 (1220 mg, 0.428 mmol, 1.0 equiv.) was added 4M HCl in dioxane (2.142 mL, 8.568 mmol, 20 equiv.) at room temperature. The reaction was kept at room temperature for 5 hrs. The reaction mixture was concentrated. The product was used directly without further purification. LC-MS: [M+3H]/3 calculated 930.89, found 932.29.

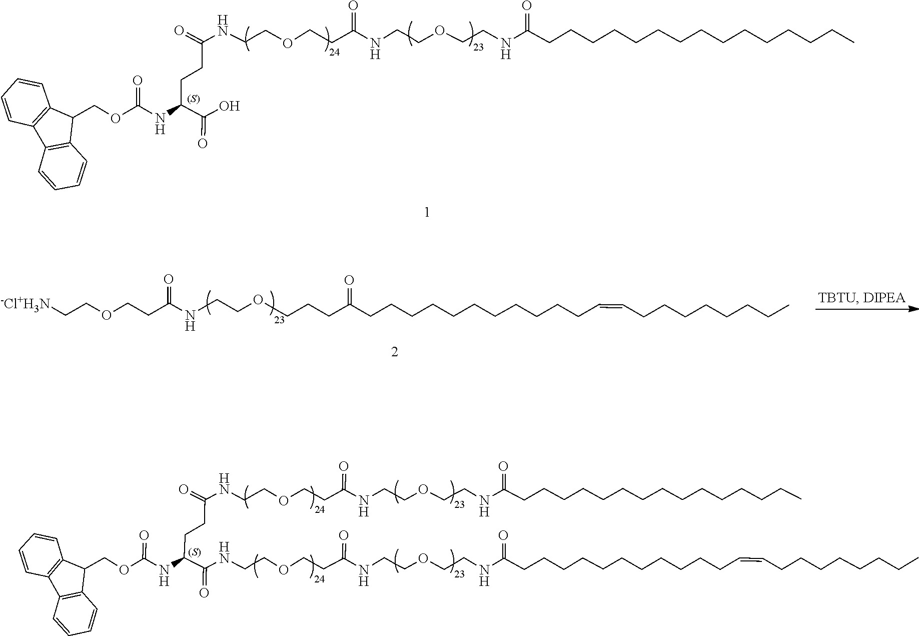

To a solution of compound 1 (800 mg, 0.286 mmol, 1.0 equiv.), compound 2 (733 mg, 0.286 mmol, 1.00 equiv.) and diisopropylethylamine (0.150 mL, 0.859 mmol, 3.0 equiv.) in DMF (10 mL) was added TBTU (110 mg, 0.344 mmol, 1.2 equiv.) at room temperature. The reaction was kept at room temperature for 3 hrs. The reaction mixture was concentrated. The product was purified by CombiFlash and was eluted with 10-20% methanol in dichloromethane. LC-MS: [M+5H]/5 calculated 1059.46, found 1060.94.

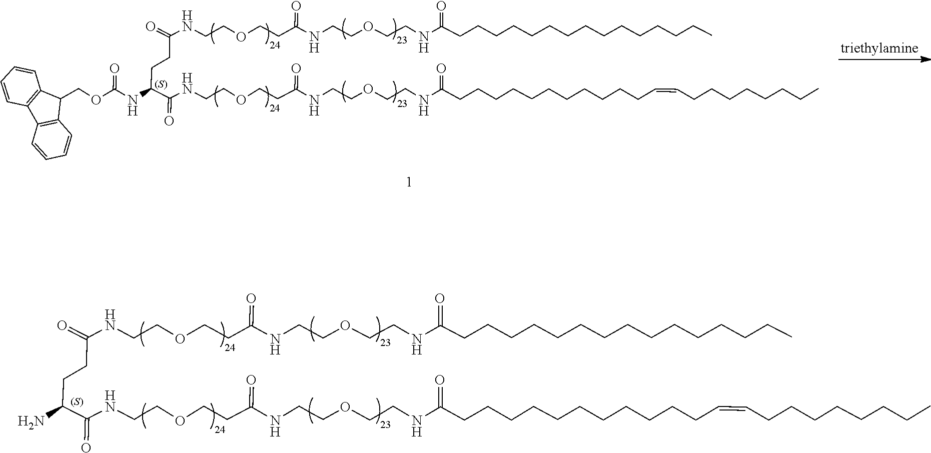

To a solution of compound 1 (914 mg, 0.172 mmol, 1.0 equiv.) in anhydrous DMF (4 mL) was added triethylamine (1 mL) at room temperature. The reaction was kept at room temperature overnight and the solvent was concentrated. The product was used directly without further purification. LC-MS: [M+5H]/5 calculated 1015.05, found 1016.41.

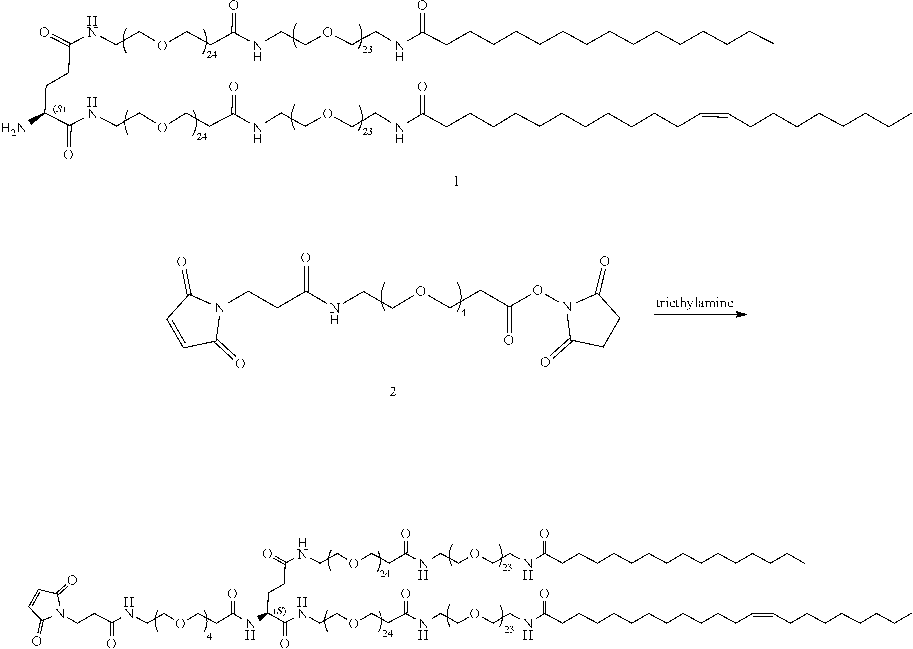

To a solution of compound 1 (875 mg, 0.172 mmol, 1.0 equiv.) and compound 2 (97.5 mg, 0.189 mmol, 1.1 equiv.) in anhydrous DCM (20 mL) was added triethylamine (0.073 mL, 0.517 mmol, 3.0 equiv.) at room temperature. The reaction was kept at room temperature overnight and the solvent was concentrated. The product was separated by CombiFlash and was eluted with 12-20% methanol in dicholoromethane. LC-MS: calculated [M+5H]/5 1094.68, found 1095.98.

### Synthesis of LP87-p

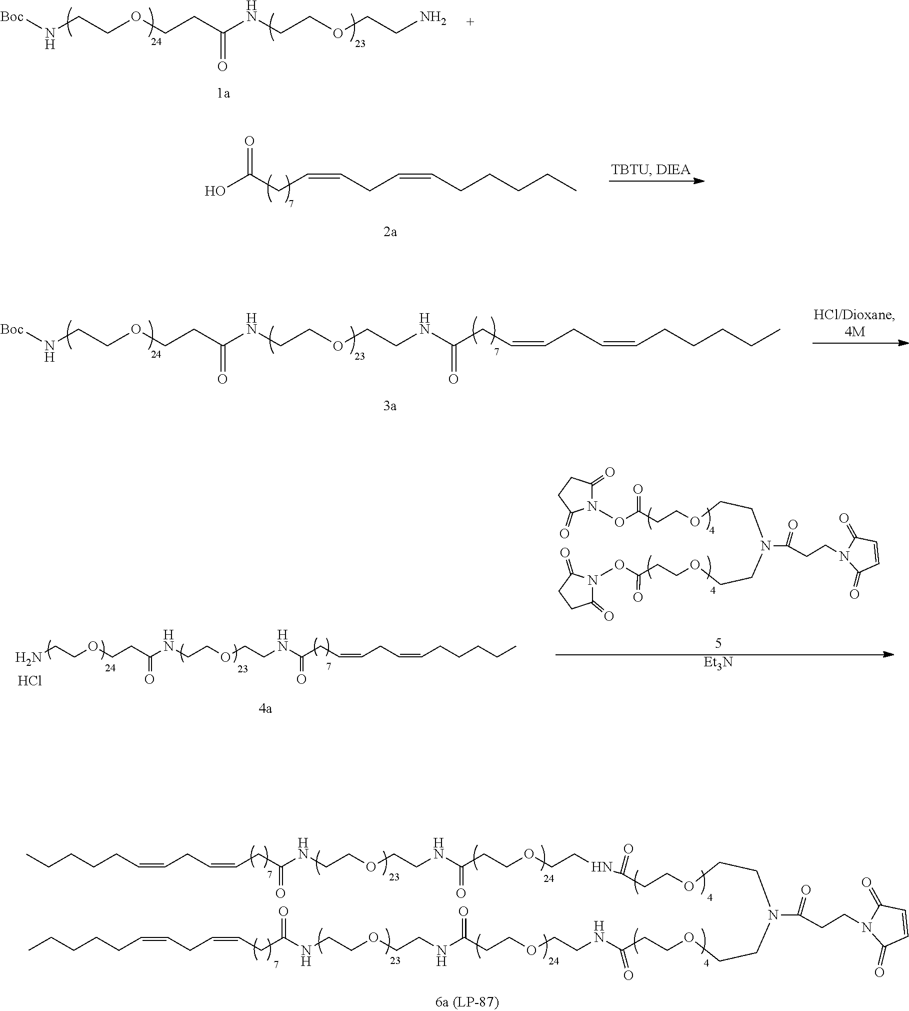

Solid TBTU (50 mg, 0.156 mmol) was added to a solution of Boc-protected PEG₄₇-amine 1a (Quanta Biodesign Limited, 300 mg, 0.13 mmol), linoleic acid 2a (37 mg, 0.13 mmol), and DIEA (68 uL mL, 0.39 mmol) in DMF (9 mL). The reaction mixture was sonicated to dissolve solids and stirred for 16 h at RT. The solvent was removed in high vacuo, toluene was evaporated twice from the residue, the residue was dissolved in chloroform (50 mL), washed with NaHCO₃(2×10 mL) and brine (10 mL). Product was dried (Na₂SO₄), concentrated in vacuo, and purified on CombiFlash (SiO₂) using the system DCM: 20% MeOH in DCM, gradient 0-80%, 20 min. Calculated MW 2564.22, (M+2×18)/2=1300.1, (M+3×18)/3=872.74 Found: MS (ES, pos): 1299.74 [M+2NH₄]²⁺, 873.04 [M+3NH₄]³⁺. Compound 3a (195 mg, 0.0764 mmol) was converted into amine hydrochloride 4b by treatment with ice-cold 4M HCl/dioxane solution (5 mL) followed by stirring at RT for 1 h. The reaction mixture was concentrated and dried in vacuo, the residual HCl was removed by 2 evaporation of toluene from the product. Dry amine hydrochloride salt was dissolved in anhydrous DMF (5 mL), Bis-NHS ester 5 (28 mg, 0.033 mmol) and Et3N (28 uL, 0.198 mmol) were added and stirred for 3h at RT. The solvent was removed in vacuo, toluene was evaporated twice from the residue and the product 6a was purified on CombiFlash using the system DCM: 20% MeOH in DCM, gradient 0-100%, 30 min. Calculated MW 5556.9, (M+3×18)/3=1870.50, (M+4×18)/4=1407.23 Found: MS (ES, pos): 1870.50 [M+3NH₄]³⁺, 1407.40 [M+4NH₄]⁴⁺.

### Synthesis of LP89-p

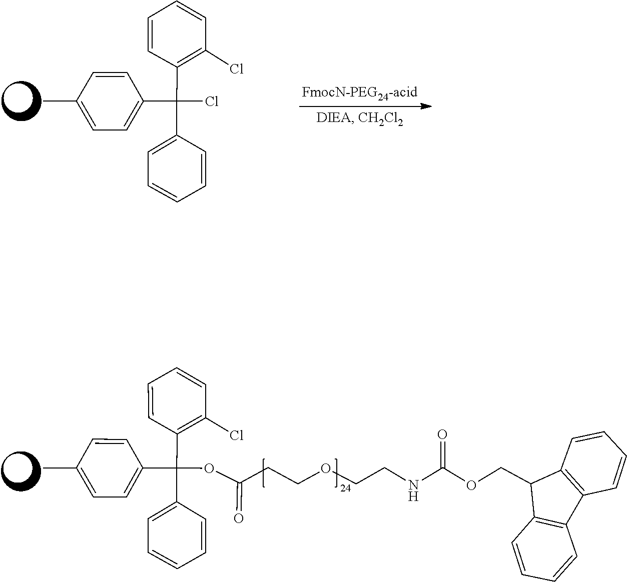

To a 25 mL fritted peptide synthesis vessel was added 2-chlorotrityl chloride resin (0.4589 g, 1.46 mmol/g, 0.670 mmol). The resin was swelled in CH₂Cl₂ and drained before adding Fmoc-N-amido-PEG₂₄-acid (0.9170 g, 0.670 mmol, 1 eq.) and diisopropylethylamine (DIEA) (0.584 mL, 3.35 mmol, 5 eq). The flask was rocked for 1 hour before adding methanol (0.367 mL, 0.8 mL/g resin) to cap any remaining trityl resin. After 40 minutes, the flask was drained, and washed with CH₂Cl₂×3, DMF x2, CH₂Cl₂×2, and MeOH x3 (approx. 5 mL each wash). The resin was dried under high-vac overnight.

Resin loading: 11.5 mg of resin was suspended in 0.8 mL DMF and swelled for 15 minutes. 0.2 mL piperidine was added and allowed to stand 15 minutes. A 10× dilution was taken up in DMF and UV-vis taken, A=2.66 (approx.). The resin loading was calculated to be 0.297 mmol/g, with a total of 919 mg of resin, for a scale of 0.273 mmol.

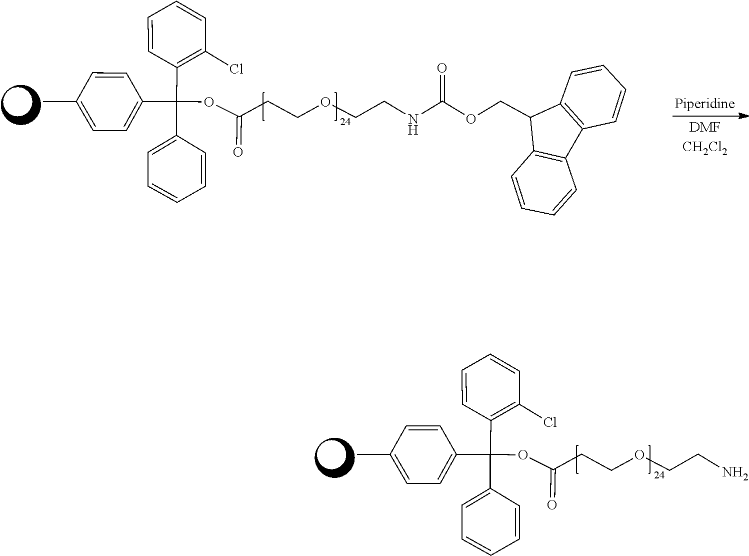

The resin was suspended in CH₂Cl₂/DMF/piperidine 1:1:2, 9.6 mL. After shaking for 30 minutes, the solution was drained, and resin washed with DMF (4×9.2 mL).

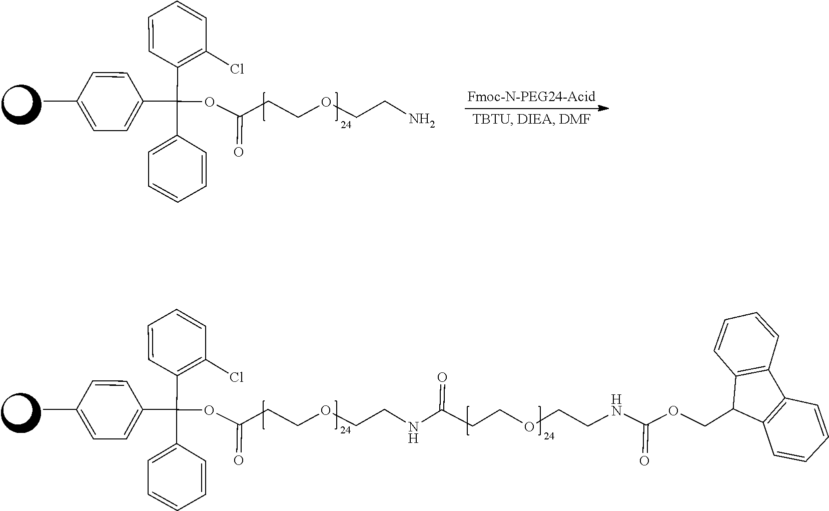

Fmoc-N-amido-PEG₂₄-acid (0.7473 g, 0.5460 mmol, 2 eq), TBTU (0.1753 g, 0.5460 mmol, 2 eq), and DIEA (0.190 mL, 1.092 mmol, 4 eq) were combined in DMF (7.6 mL) and mixed for 2-3 minutes before the solution was added to the resin in the synthesis flask. The flask was shaken for 1 h, after which a yellow orange solution was drained from the orange resin. The resin was washed with DMF and MeOH (3×8.6 mL each) then dried overnight under high-vac. 1.277 g resin, theoretical 1.227 g. Product masses were observed by LC-MS following a microcleavage.

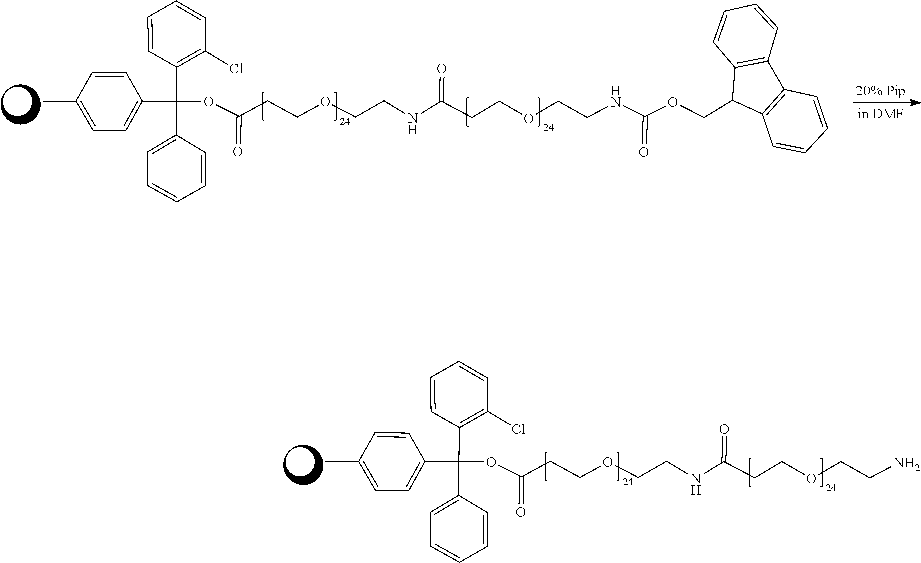

The resin was treated with 20% piperidine in DMF (12.3 mL) for 30 minutes, then washed with DMF (4×12.3 mL).

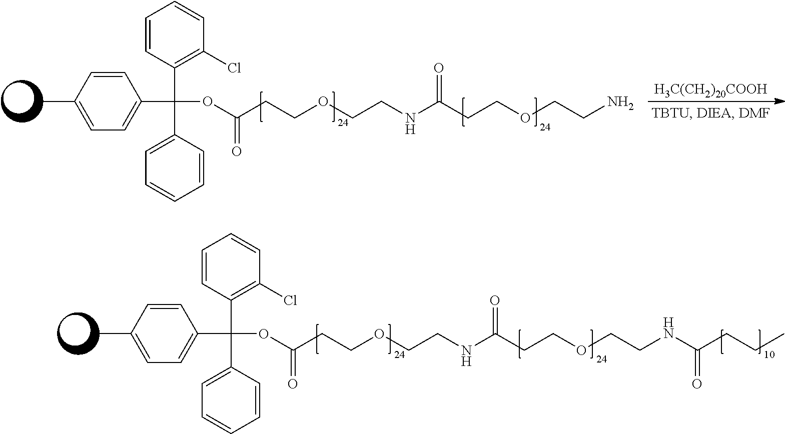

Behenic acid (0.186 g, 0.546 mmol, 2.0 eq), TBTU (0.175 g, 0.546 mmol, 2 eq) and DIEA (0.190 mL, 1.092 mmol, 4 eq) were dissolved in DMF (10.7 mL). The solution was added to the resin and solution vial rinsed with DMF, then combined (2×1 mL). The mixture was shaken for 75 minutes then drained and washed with DMF, THF, and MeOH (3×13 mL each). The resin was dried under high-vac (90 minutes). 1.351 g obtained, theoretical 1.254 g. Product masses (and no starting material masses) were observed in LC-MS following a microcleavage.

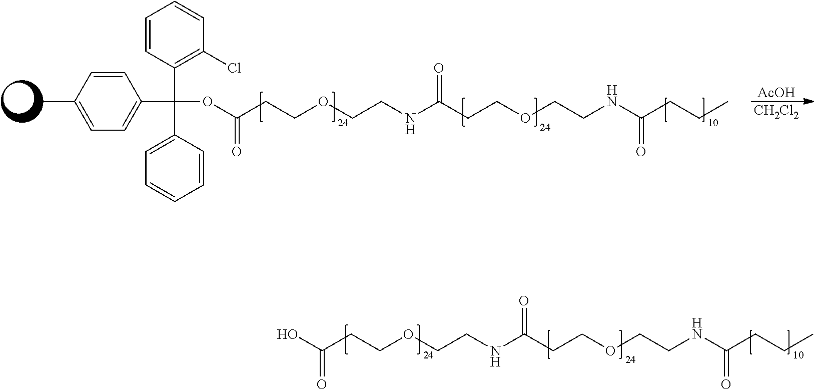

The resin was treated with CH₂Cl₂ (11 mL) and AcOH (1.1 mL) for 30 minutes, then drained. This cleavage was repeated a total of 4 times, then the resin was treated with 8 mL CH₂Cl₂, 1 mL AcOH, and 1 mL 2,2,2-trifluoroethanol, shaken for 30 minutes, and drained. This cleavage was repeated a second time. The solutions from all cleavages were combined and concentrated to yield 530.8 mg, which was purified by column chromatography.

The crude compound was loaded onto a silica column (24 g) and eluted 0-20% McOH in CH₂Cl₂. Clean fractions were combined to yield 69.9 mg of target compound.

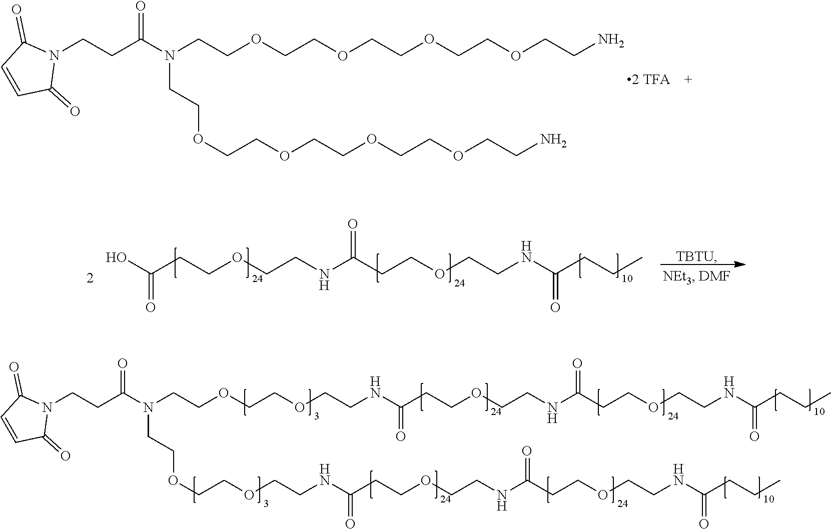

To a vial was added N-mal-N-bis(PEG₄)amine TFA salt (10.7 mg, 0.0128 mmol, 1 eq), acid-PEG₂₄-amido-PEG₂₄-C₂₂ (69.9 mg, 0.0269 mmol, 2.1 eq), TBTU (10.3 mg, 0.0320 mmol, 2.5 eq), NEt₃ (5.4 uL, 0.0385 mmol, 3 eq), and CH₂Cl₂ (1 mL). The reaction was stirred for 24 h, then NEt₃ (5.4 uL, 0.0385 mmol, 3 eq) was added. After approximately 50 hours, the reaction was concentrated and purified by column chromatography, 0-30% MeOH in CH₂Cl₂, to obtain 32.8 mg of product (44%).

### Synthesis of LP90-p

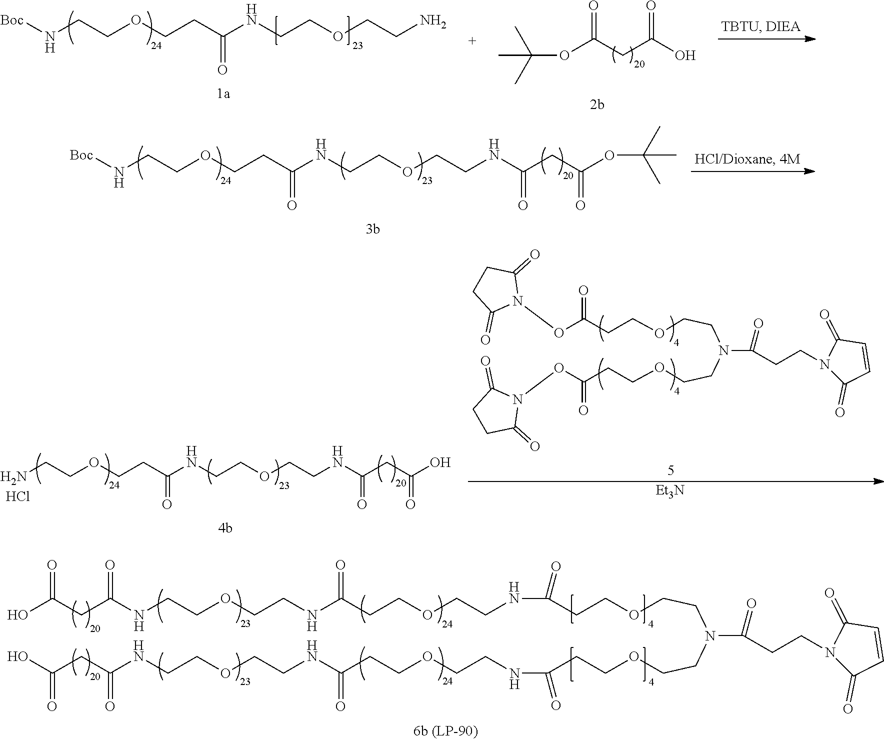

Solid TBTU (50 mg, 0.156 mmol) was added to a solution of Boc-protected PEG-amine 1a (Quanta Biodesign Limited, 300 mg, 0.13 mmol), mono-protected docosanedioic acid 2b (56 mg, 0.13 mmol), and DIEA (68 uL mL, 0.39 mmol) in DMF (9 mL). The reaction mixture was stirred for 16 h at RT. The solvent was removed in vacuo and toluene was evaporated 3 times from the residue. The residue was taken in DCM 30 (mL), mixed with Sift (1.6 g), and loaded on CombiFlash. The product was purified using the system DCM: 20% McOH in DCM, gradient 0-100%, 45 min. Calculated MW 2710.45, (M+2×18)/2=1373.22, (M+3×18)/3=921.48 Found: MS (ES, pos): 1373.18 [M+2NH₄]²⁺, 921.37 [M+3NH₄]³⁺.

Compound 3b (238 mg, 0.088 mmol) was converted into amino acid hydrochloride 4b by treatment with ice-cold 4M HCl/dioxane solution (6 mL) followed by stirring at RT for 4 h. The reaction mixture was concentrated and dried in vacuo, the residual HCl was removed by 2 evaporation of toluene from the residue.

Dry amine hydrochloride salt 4b was dissolved in anhydrous DCM (5 mL), Bis-NHS ester 5 (34.2 mg, 0.04 mmol) and Et3N (55 uL, 0.4 mmol) were added and stirred for 3h at RT. The solvent was removed in vacuo, the product 6b was purified on CombiFlash using the system DCM: 20% MeOH in DCM, gradient 0-100%, 40 min. Calculated MW 5737.13, (M+3×18)/3=1930.38, (M+4×18)/4=1452.28 Found: MS (ES, pos): 1930.45 [M+3NH₄]³⁺1452.29 [M+4NH₄]⁴⁺.

### Synthesis of LP91-p

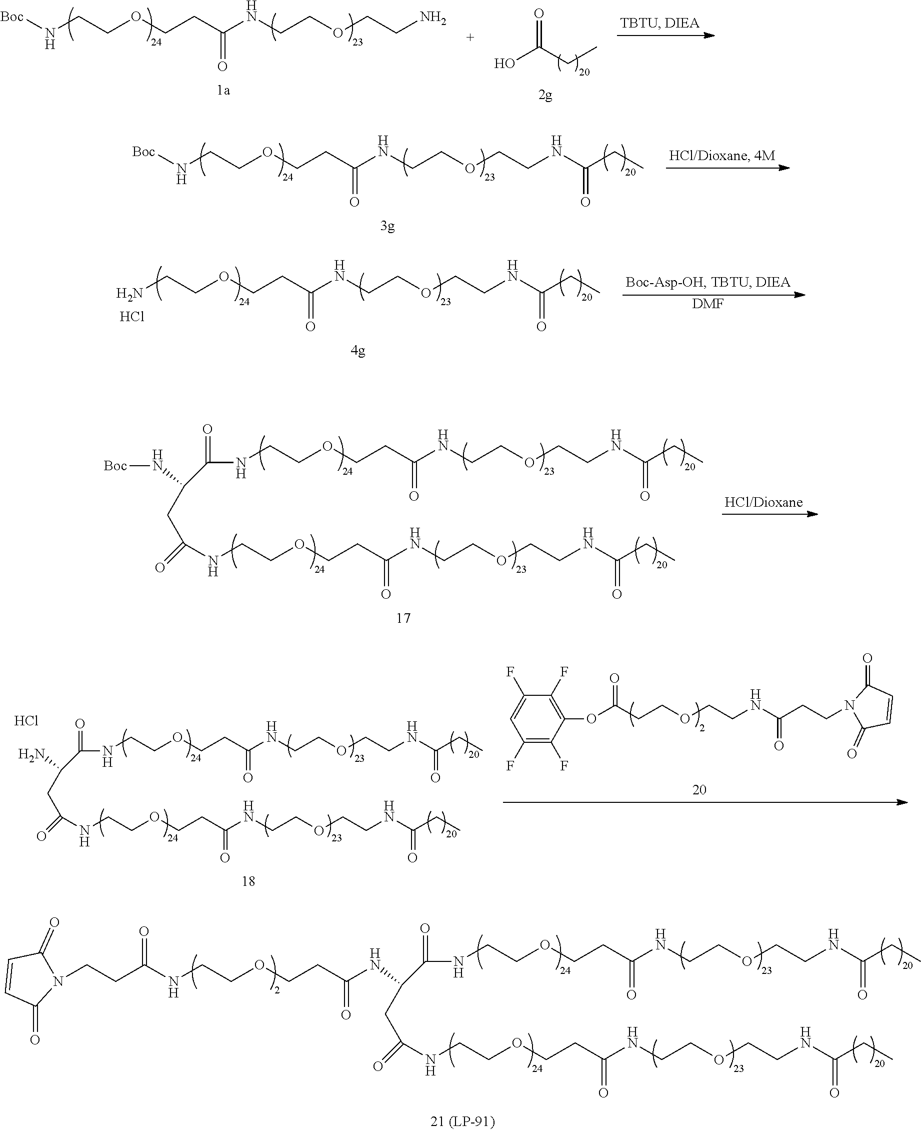

Solid TBTU (335 mg, 1.043 mmol) was added to a solution of Boc-protected PEG₄₇-amine 1a (2 g, 0.869 mmol), behenic acid acid 2 g (296 mg, 0.87 mmol), and DIEA (454 uL mL, 2.067 mmol) in DMF (16 mL). The reaction mixture was sonicated to dissolve solids and stirred for 16 h at RT. The solvent was removed in high vacuo, toluene was evaporated twice from the residue, the residue was dissolved in chloroform (150 mL), washed with NaHCO₃(2×30 mL) and brine (30 mL). Product 3 g was dried (Na₂SO₄), concentrated in vacuo, and purified on CombiFlash (SiO₂) using the system DCM: 20% MeOH in DCM, gradient 0-80%, 35 min. Calculated MW 2624.36, (M+2×18)/2=1330.18, (M+3×18)/4=892.79. Found: MS (ES, pos): 1330.58 [M+2NH₄]²⁺, 893.21 [M+3NH₄]³⁺.

3 g (1.862 g) was converted into amine hydrochloride 4 g by treatment with 4 M HCl in dioxane solution (10 mL) as described in the procedure for LP39, above.

An aliquot of dry salt 4 g (227 mg, 0.089 mmol)) was brought into reaction with Boc-Asp-OH (10 mg, 0.043 mmol), TBTU (32 mg, 0.099 mmol), and DIEA (96 uL, 0.55 mmol) as it was described in preparation of LP54 to obtain compound 17, yield 152 mg (0.029 mmol). This product was treated with HCl/dioxane solution as in preparation of 14c as described in the synthesis of LP54, above, to obtain hydrochloride salt 18 (yield 100%), and used directly in the following step. Calculated MW 5145.57, (M+3)/3=1716.19, (M+4)/4=1287.23. Found: MS (ES, pos): 1715.91 [M+3H]³⁺, 1287.23 [M+4H]⁴⁺.

Hydrochloride salt 18 (0.029 mmol) was brought into reaction with terafluorophenyl ester 20 (Quanta Biodesign, 15 mg, 0.032 mmol) and Et3N (12 uL, 0.087 mmol) as it was described for 16c in the synthesis of LP54, above. The product 21 was purified on CombiFlash. Yield 40 mg. Calculated MW 5455.87, (M+4)/4=1364.97, (M+5)/5=1092.17. Found: MS (ES, pos): 1364.66 [M+4H]⁴⁺, 1092.05 [M+4H]⁴⁺.

### Synthesis of LP92-p

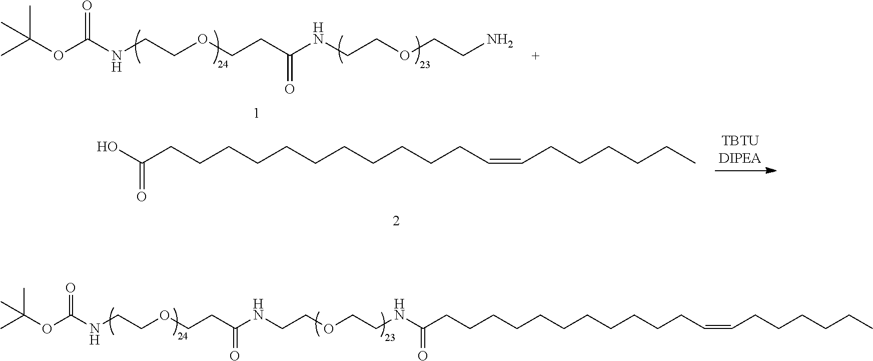

To a solution of compound 1 (140 mg, 0.0608 mmol, 1.0 equiv.), compound 2 (20.8 mg, 0.0669 mmol, 1.1 equiv.) and diisopropylethylamine (0.032 mL, 0.182 mmol, 3.0 equiv.) in anhydrous DMF (3 mL) was added TBTU (23.4 mg, 0.073 mmol, 1.2 equiv.) at room temperature. The reaction was kept at room temperature for 2 hrs. The reaction mixture was concentrated. The product was purified by CombiFlash and was eluted with 12-18% methanol in dichloromethane. LC-MS: calculated [M+2H]+/2 1297.33, found 1297.19.

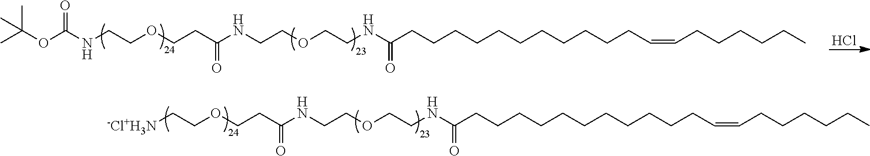

To solid of compound 1(90 mg, 0.0347 mmol, 1.0 equiv.) was added HCl solution in dioxane (0.434 mL, 1.734 mmol, 50 equiv.) at room temperature. The reaction was kept at room temperature for 30 min and the solvent was concentrated. The product was used directly without further purification. LC-MS: calculated [M+2H]+/2 1247.30, found 1247.98.

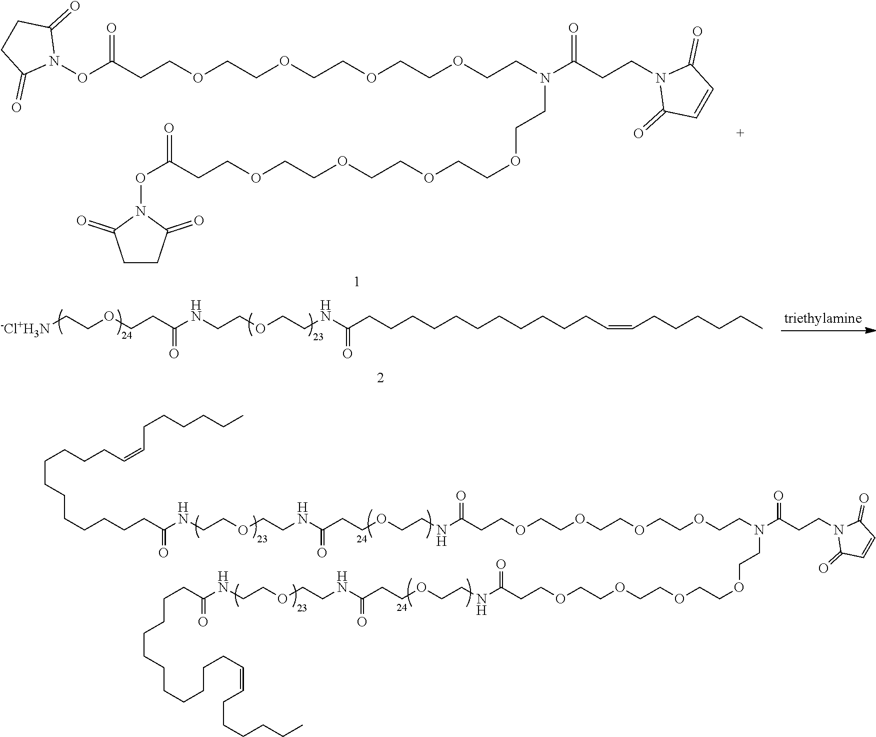

To a solution of compound 1 (14 mg, 0.0163 mmol, 1.0 equiv.) and compound 2 (84.6 mg, 0.0334 mmol, 2.05 equiv.) in anhydrous DCM (2 mL) was added triethylamine (0.012 mL, 0.0815 mmol, 5.0 equiv.) at room temperature. The reaction was kept at room temperature for 1 hr and the solvent was concentrated. The product was separated by CombiFlash and was eluted with 12-18% methanol in dicholoromethane. LC-MS: calculated [M+5H]+/5 1123.70, found 1124.10, calculated [M+6H]+/6 936.58, found 937.22.

### Synthesis of LP93-p

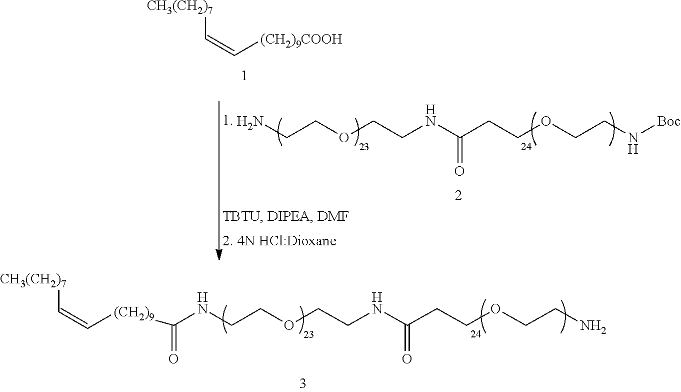

To cis-11-eicosenoic acid 1 (30 mg, 0.0979 mmol) in a solution of Boc-PEG₄₇-NH_{2 2} (223 mg, 0.1 mmol) in DMF (2.0 mL) was added TBTU (37.2 mg, 0.115 mmol) and DIPEA (50 uL). After stirring the resulting suspension overnight, water was added. The mixture was extracted using DCM:20% TFE and the combined organic phases were dried over Na₂SO₄. After filtration, the solvent was removed under vacuum to dryness and the crude product was purified by flash chromatography (20% MeOH in DCM). To the product was added 2 mL of 4N HCl:Dioxane under anhydrous conditions until the deprotection was determined to be complete by LC-MS: calculated [M+H]+for 2550.28 m/z, found 2551.

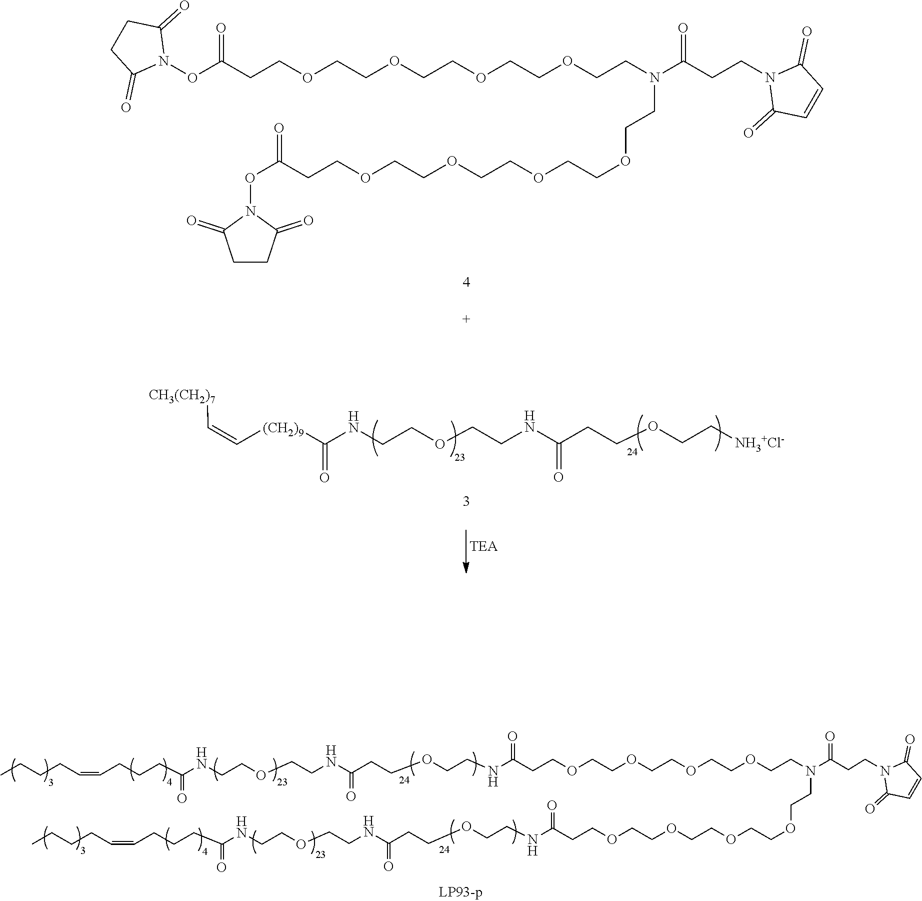

To a solution of compound 4 (19 mg, 0.0221 mmol, 1.0 equiv.) and compound 3 (16 mg, 0.0454 mmol, 2.05 equiv.) in anhydrous DCM (2 mL) was added triethylamine (16 uL, 0.1106 mmol, 5.0 equiv.) at room temperature. The reaction mixture was kept at room temperature overnight and the solvent was removed under vacuum. LP93-p was purified by CombiFlash® eluting with 10-17% methanol in dichloromethane.

### Synthesis of LP94-p

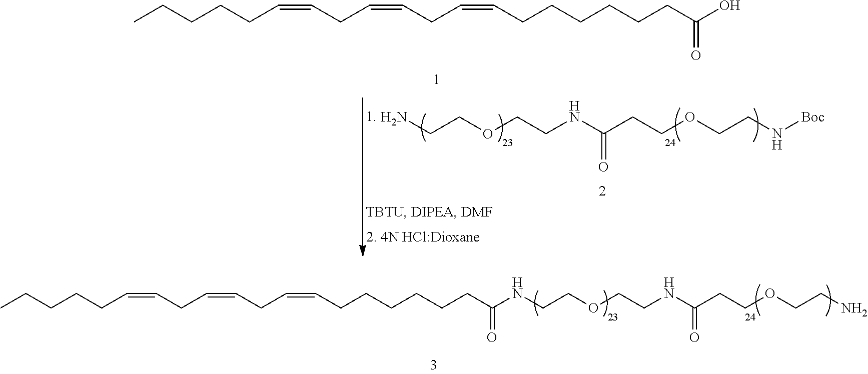

To dihomo-γ-linolenic acid 1 (30 mg, 0.0979 mmol) in a solution of DMF (2.0 mL) was added Boc-PEG₄₇-NH_{2 2} (225 mg, 0.1 mmol), TBTU (37.7 mg, 0.117 mmol) and DIPEA (50 uL). After stirring the resulting suspension overnight, water was added. The mixture was extracted using DCM:20% TFE and the combined organic phases were dried over Na₂SO₄. After filtration, the solvent was concentrated to dryness and the crude product was purified by flash chromatography (DCM:20% MeOH). To the product was added 2 mL of 4N HCl:Dioxane under anhydrous conditions until the deprotection was determined to be complete by LC-MS: calculated [M+H]+for 2560.28 m/z, found 2561.01.

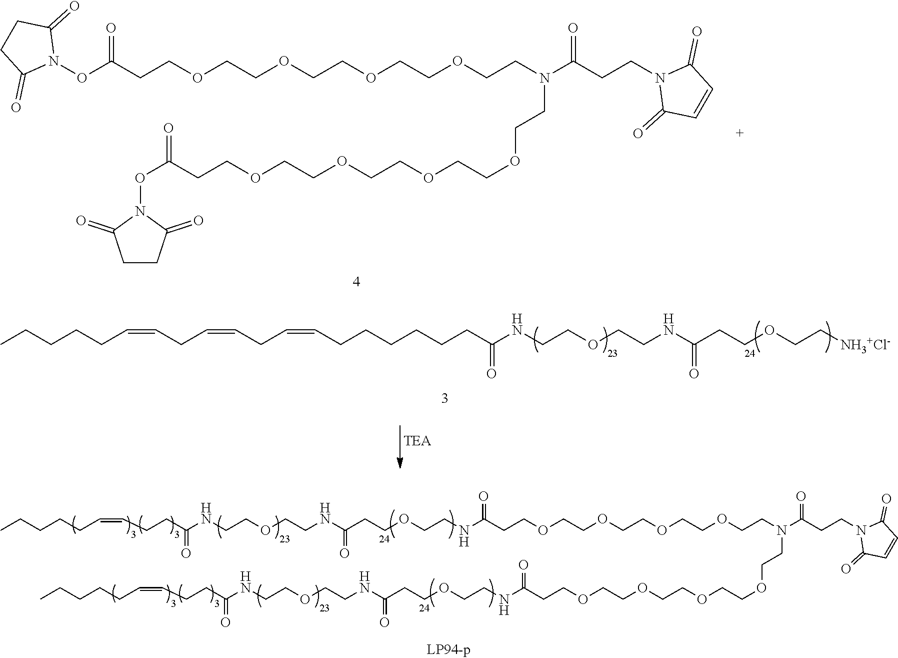

To a solution of compound 4 (19 mg, 0.0221 mmol, 1.0 equiv.) and compound 3 (112 mg, 0.0454 mmol, 2.05 equiv.) in anhydrous DCM (2 mL) was added triethylamine (16 uL, 0.1106 mmol, 5.0 equiv.) at room temperature. The reaction mixture was kept at room temperature overnight and the solvent was removed under vacuum. LP94-p was separated by CombiFlash® eluting with 10-17% methanol in dichloromethane.

### Synthesis of LP95-p

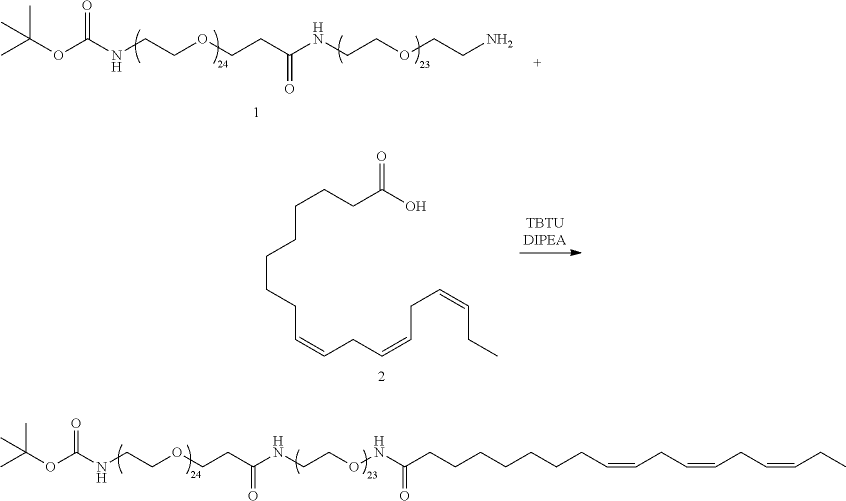

To a solution of compound 1 (150 mg, 0.0652 mmol, 1.0 equiv.), compound 2 (20 mg, 0.0717 mmol, 1.1 equiv.) and diisopropylethylamine (0.034 mL, 0.195 mmol, 3.0 equiv.) in anhydrous DMF (3 mL) was added TBTU (25.1 mg, 0.0782 mmol, 1.2 equiv.) at room temperature. The reaction was kept at room temperature for 2 hrs. The reaction mixture was concentrated. The product was purified by CombiFlash and was eluted with 12-18% methanol in dichloromethane. LC-MS: calculated [M+2H]+/2 1281.30, found 1281.71.

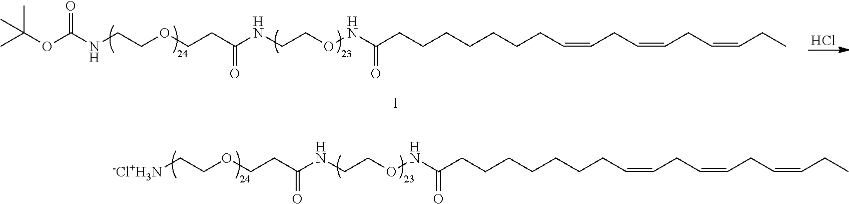

To solid of compound 1 (80 mg, 0.0312 mmol, 1.0 equiv.) was added HCl solution in dioxane (0.390 mL, 1.561 mmol, 50 equiv.) at room temperature. The reaction was kept at room temperature for 30 min and the solvent was concentrated. The product was used directly without further purification. LC-MS: calculated [M+2H]+/2 1231.27, found 1231.65.

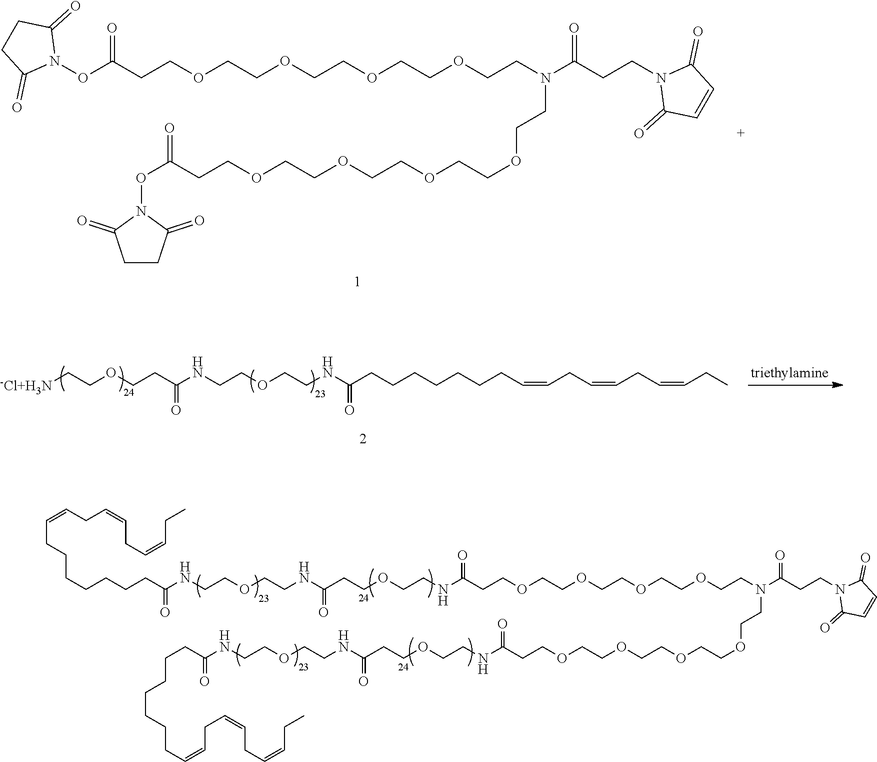

To a solution of compound 1 (13 mg, 0.0151 mmol, 1.0 equiv.) and compound 2 (77.5 mg, 0.0310 mmol, 2.05 equiv.) in anhydrous DCM (2 mL) was added triethylamine (0.011 mL, 0.0757 mmol, 5.0 equiv.) at room temperature. The reaction was kept at room temperature for 1 hr and the solvent was concentrated. The product was separated by CombiFlash and was eluted with 12-18% methanol in dicholoromethane. LC-MS: calculated [M+5H]+/5 1110.88, found 1111.62, calculated [M+6H]+/6 925.90, found 926.41.

### Synthesis of LP101-p

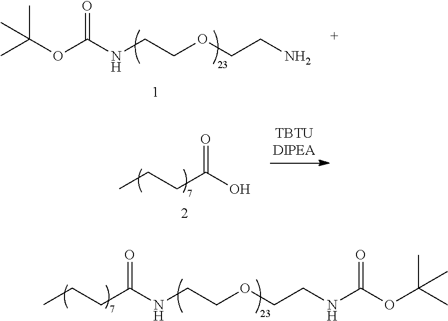

To a solution of compound 1 (250 mg, 0.213 mmol, 1.0 equiv.), compound 2 (65 mg, 0.255 mmol, 1.20 equiv.) and diisopropylethylamine (0.111 mL, 0.629 mmol, 3.0 equiv.) in anhydrous DMF (3 mL) was added TBTU (102 mg, 0.319 mmol, 1.2 equiv.) at room temperature. The reaction was kept at room temperature overnight. The product was purified by CombiFlash and was eluted with 6-12% methanol in dichloromethane. LC-MS: calculated [M+H]+ 1411.95, found 1411.95.

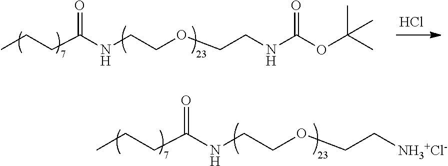

To solid of compound 1(200 mg, 0.141 mmol, 1.0 equiv.) was added HCl solution in dioxane (0.708 mL, 2.833 mmol, 20 equiv.) at room temperature. The reaction was kept at room temperature for 1 hr and the solvent was concentrated. The product was used directly without further purification. LC-MS: calculated [M+H]+ 1311.90, found 1312.32.

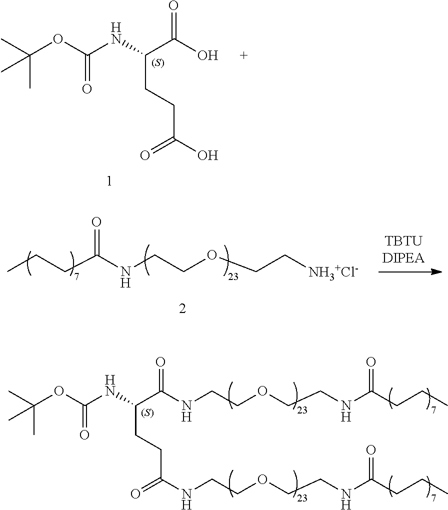

To a solution of compound 1 (100 mg, 0.0404 mmol, 1.0 equiv.), compound 2 (111 mg, 0.0829 mmol, 2.05 equiv.) and diisopropylethylamine (35 mL, 0.202 mmol, 3.0 equiv.) in anhydrous DMF (3 mL) was added TBTU (32.5 mg, 0.101 mmol, 2.5 equiv.) at room temperature. The reaction was kept at room temperature overnight. The reaction mixture was concentrated. The product was purified by CombiFlash and was eluted with 6-10% methanol in dichloromethane. LC-MS: calculated [M+2H]+/2 1417.44, found 1418.19.

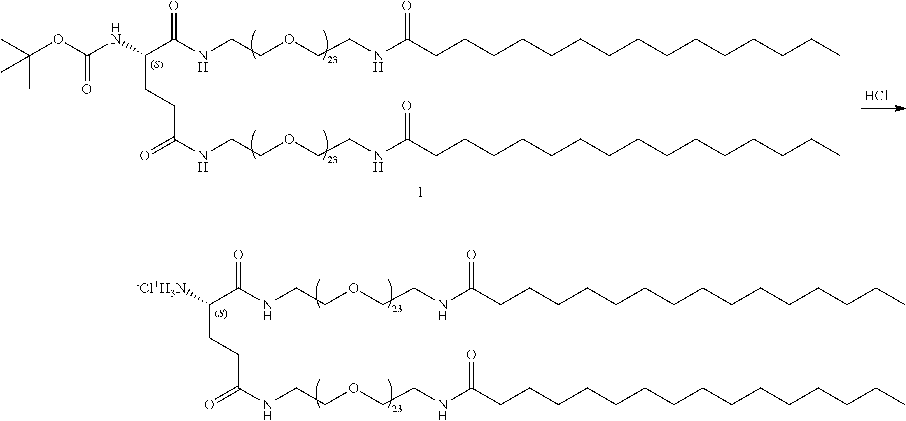

To compound 1 (80 mg, 0.0282 mmol, 1.0 equiv.) was added 4M HCl in dioxane (0.353 mL, 1.411 mmol, 50 equiv.) at room temperature. The reaction was kept at room temperature for 1 hr. The reaction mixture was concentrated. The product was used directly without further purification. LC-MS: [M+2H]/2 calculated 1367.41, found 1368.26.

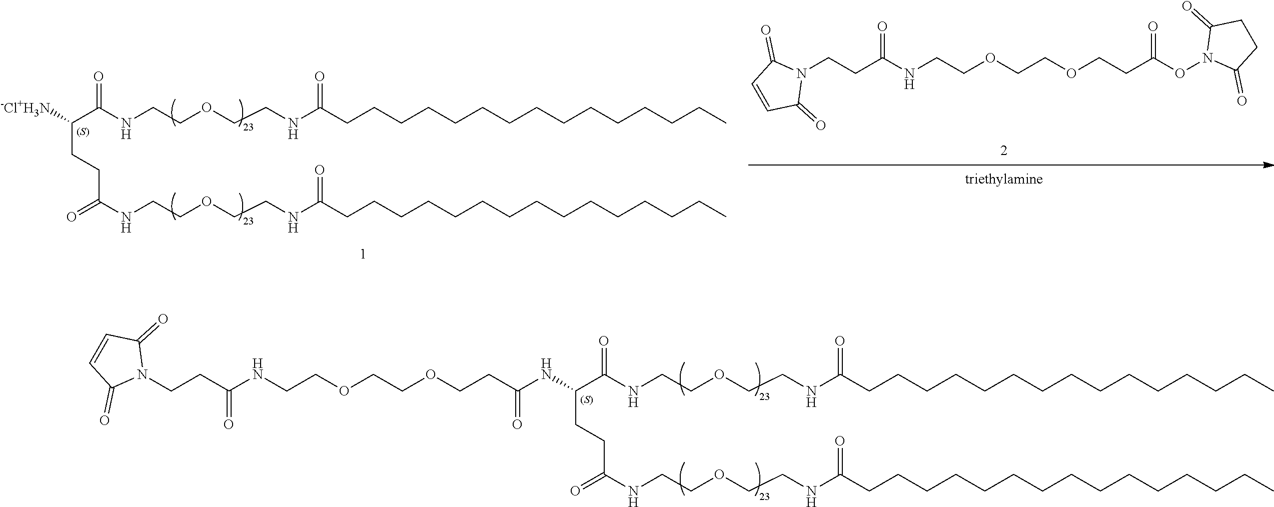

To a solution of compound 1 (78 mg, 0.0281 mmol, 1.0 equiv.) and compound 2 (12 mg, 0.0281 mmol, 1.0 equiv.) in anhydrous DCM (2 mL) was added triethylamine (0.020 mL, 0.140 mmol, 5.0 equiv.) at room temperature. The reaction was kept at room temperature overnight and the solvent was concentrated. The product was separated by CombiFlash and was eluted with 12-20% methanol in dicholoromethane. LC-MS: calculated [M+3H]/3 1015.31, found 1015.71.

### Synthesis of LP102-p

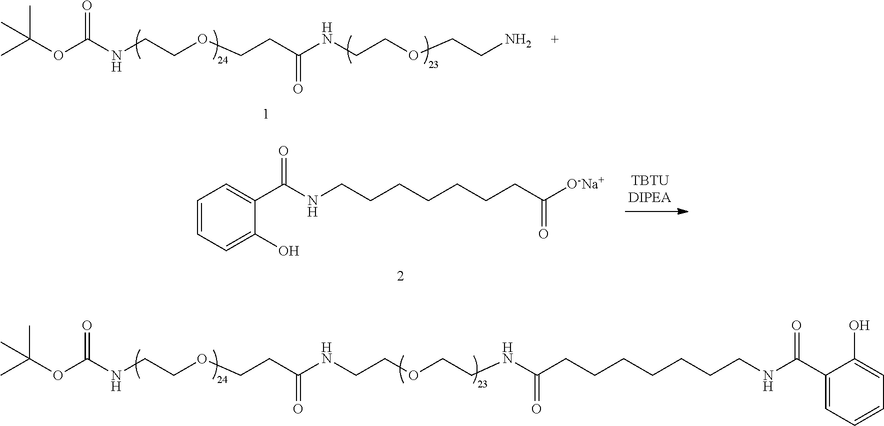

To a solution of compound 1 (124 mg, 0.0539 mmol, 1.0 equiv.), compound 2 (19.5 mg, 0.0646 mmol, 1.2 equiv.) and diisopropylethylamine (0.028 mL, 0.161 mmol, 3.0 equiv.) in anhydrous DMF (2 mL) was added TBTU (20.8 mg, 0.0646 mmol, 1.2 equiv.) at room temperature. The reaction was kept at room temperature for 1 hr. The reaction mixture was quenched with saturated sodium bicarbonate aqueous solution. The aqueous phase was extracted with DCM (3×10 mL), and the organic phase was combined, dried over Na₂SO₄, and concentrated. The product was purified by CombiFlash and was eluted with 10-12% methanol in dichloromethane. LC-MS: calculated [M+2H]+/2 1281.76, found 1282.19.

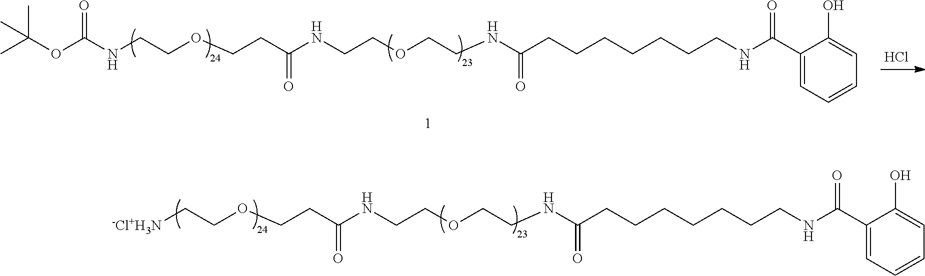

To compound 1 (66 mg, 0.0257 mmol, 1.0 equiv.) was added 4M HCl in dioxane (0.322 mL, 1.287 mmol, 50 equiv.) at room temperature. The reaction was kept at room temperature for 1 hr. The reaction mixture was concentrated. The product was used directly without further purification. LC-MS: [M+2H]/2 calculated 1231.75, found 1232.01.

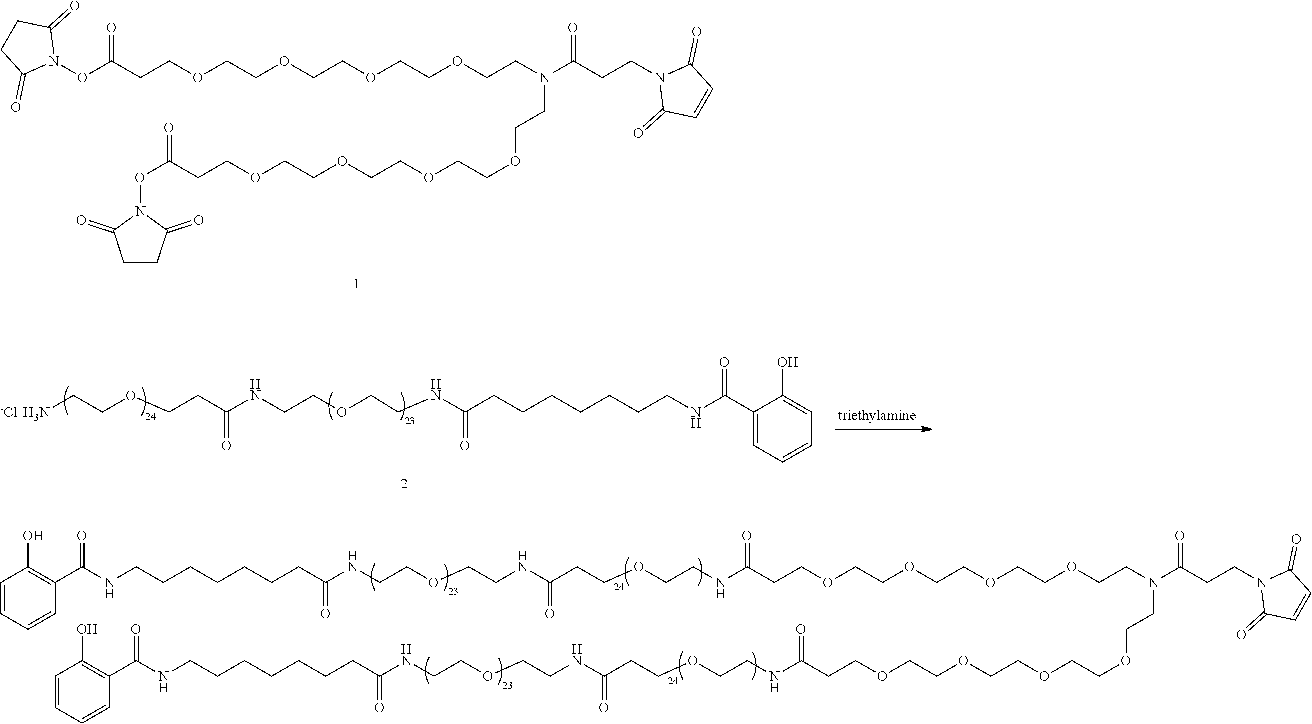

To a solution of compound 1 (11 mg, 0.0128 mmol, 1.0 equiv.) and compound 2 (64 mg, 0.0256 mmol, 2.00 equiv.) in anhydrous DCM (2 mL) was added triethylamine (0.009 mL, 0.064 mmol, 5.0 equiv.) at room temperature. The reaction was kept at room temperature for 1 hr and the solvent was concentrated. The product was separated by CombiFlash and was eluted with 12-18% methanol in dicholoromethane. LC-MS: calculated [M+6H]+/6 926.20, found 926.41.

### Synthesis of LP103-p

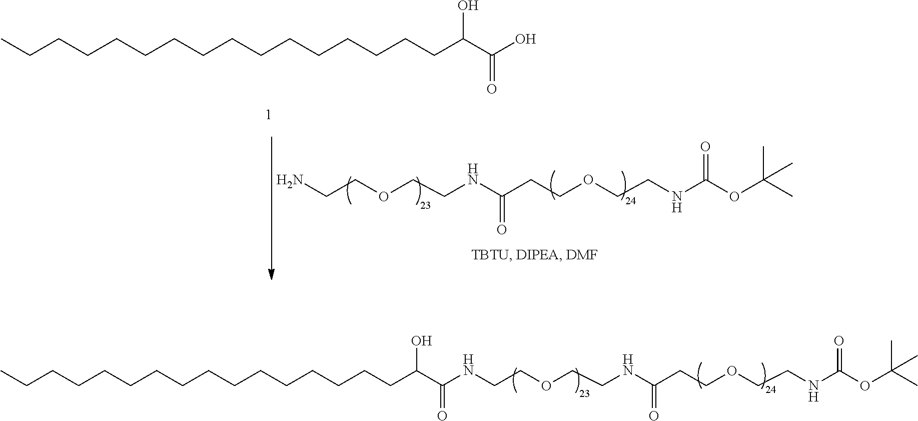

To compound 1 (35 mg, 0.1170 mmol) in a solution DMF (2.0 mL) was added Boc-PEG₄₇-NH₂ (269 mg, 0.1170 mmol), TBTU (45.1 mg, 0.1404 mmol) and DIPEA (60 uL). After stirring the suspension overnight, water was added and extracted using DCM:20% TFE and dried over Na₂SO₄. After filtration, the solvent was concentrated to dryness and the crude product was purified by flash chromatography (DCM:20% MeOH). To this was added 2 mL of 4N HCl:Dioxane and stirred under anhydrous conditions until determined complete by LC-MS: calculated [M+H]+for 2483.59 m/z, found 2484.01.

### Synthesis of LP104-p

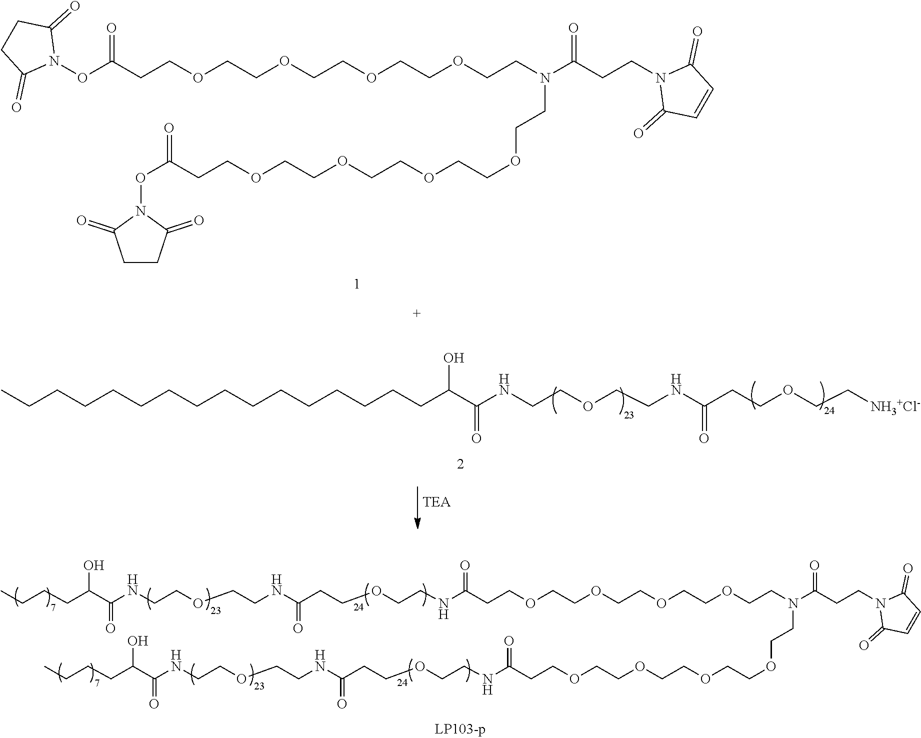

To a solution of compound 4 (10 mg, 0.0116 mmol, 1.0 equiv.) and compound 5 (59.3 mg, 0.0239 mmol, 2.05 equiv.) in anhydrous DCM (2 mL) was added triethylamine (8 uL, 0.0582 mmol, 5.0 equiv.) at room temperature. The reaction mixture was kept at room temperature overnight and the solvent was removed under vacuum. LP103-p was separated by CombiFlash® eluting with 10-17% methanol in dichloromethane. LC-MS: calculated [M+6H]+/6 933, found 934, calculated [M+7H]+/7 800, found 801.

### Synthesis of LP104-p

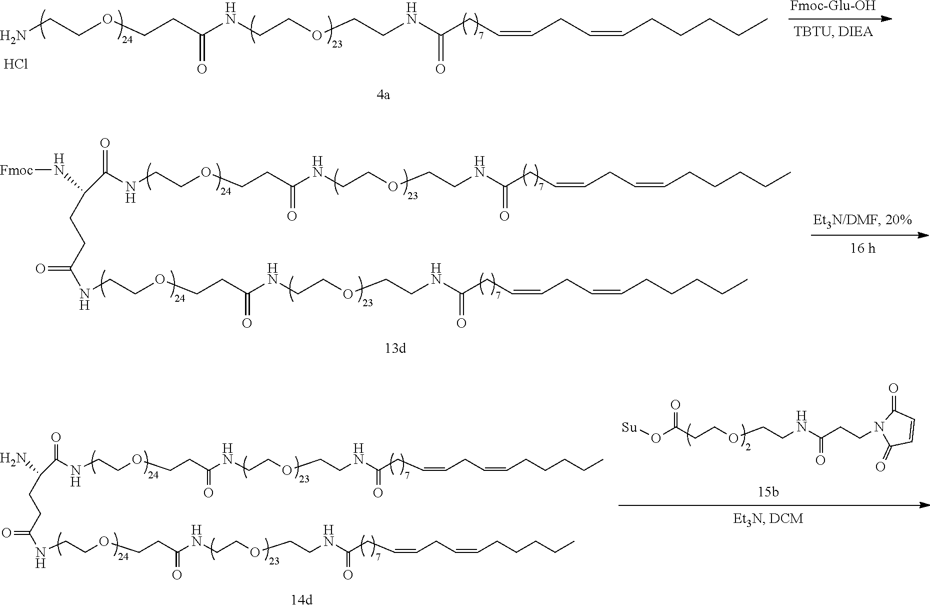

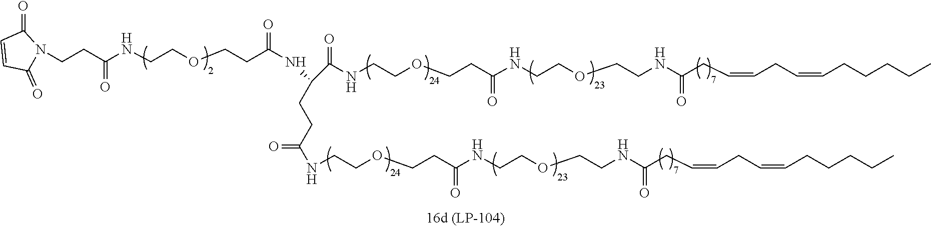

Compound 4a (synthesis shown in procedures for LP87, above), was conjugated with Fmoc-Glu-OH as described in the procedure for LP54, above. Calculated MW 5261.56, (M+3×18)/3=1771.86, (M+4×18)/4=1333.39 Found: MS (ES, pos): 1771.98 [M+3NH₄]³⁺, 1333.57 [M+4NH₄]⁴⁺.

Compound 13d was Fmoc-deprotected as described for compound 14a in the synthesis of LP39, above. The resulting product 14d was conjugated with activated ester compound 15b as described in the procedure for synthesizing compound 16a in the synthesis of LP39, above. The product was isolated following CombiFlash purification. Calculated MW 5349.62, (M+3×18)/3=1801.21, (M+4×18)/4=1355.41. Found: MS (ES, pos): 1801.87 [M+3NH₄]³⁺, 1355.92 [M+4NH₄]⁴⁺.

### Synthesis of LP106-p

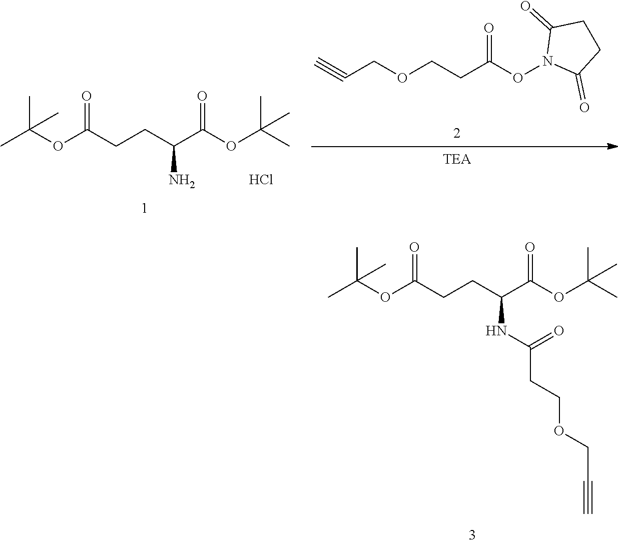 [SMILES: CC(C)(C)OC(=O)CC[C@H](N)C(=O)OC(C)(C)C ; Cl ; C#CCOCCC(=O)ON1C(=O)CCC1=O ; C#CCOCCC(=O)N[C@@H](CCC(=O)OC(C)(C)C)C(=O)OC(C)(C)C]

To compound 1 (200 mg, 0.676 mmol) in DCM (4 mL) was added TEA (218 uL, 1.56 mmol) then compound 2 (198 mg, 0.879 mmol) and the mixture was stirred at room temperature for 1 hour. Upon completion all volatiles were removed and crude compound 3 was deprotected using 4N HCl and was used subsequently without further purification.

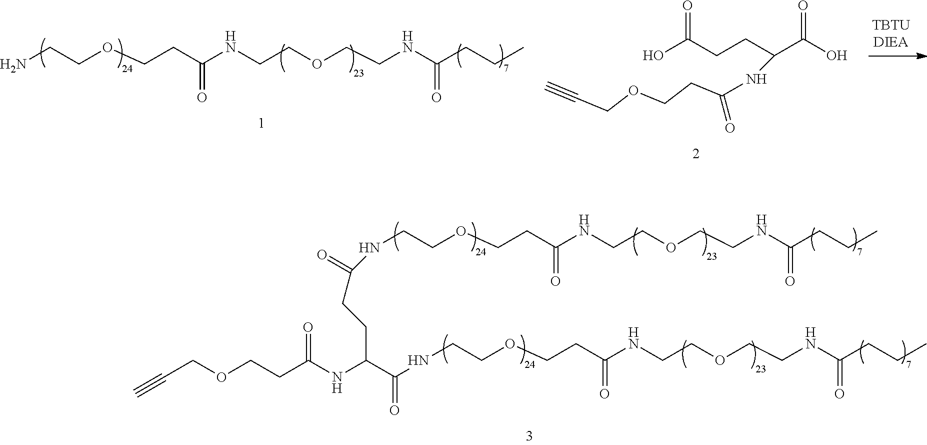

Crude compound 2 (60 mg, 0.1014 mmol assumed) was dissolved in DMF (1 mL), treated with TBTU (71.6 mg, 0.223 mmol) and stirred for 5 minutes. Compound 1 (668 mg, 0.273 mmol) and DIEA (91.8 uL, 0.527 mmol) in DMF (1 mL) were subsequently added the mixture was left to stir at room temperature for 16 hours. Upon completion all volatiles were removed and compound 3 was isolated eluting a gradient of MeOH (0.1% TFA) in water (0.1% TFA) using a phenomenex C₁₈ gemini column (10u, 50 mm×250 mm).

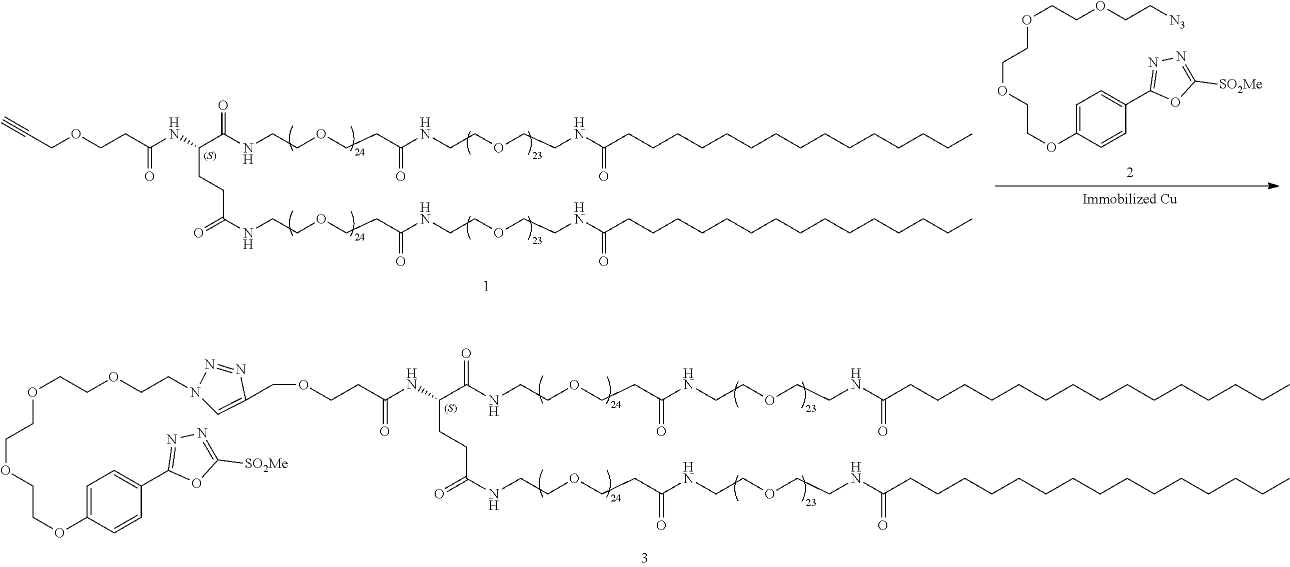

Compound 1 (23.5 mg, 0.0532 mmol) and Compound 2 (29.9 mg, 0.0586 mmol) were dissolved in 12.0 mL DMF and the vessel was sparged with N₂ for 5 minutes. Then, immobilized copper (337 mg, 0.0532 mmol) and sodium ascorbate (31.6 mg, 0.1597 mmol) were added and the reaction was stirred at 40° C. overnight.

The resin and other solids were filtered off. The filtrate was concentrated in vacuo and purified by HPLC.

### Synthesis of LP107-p

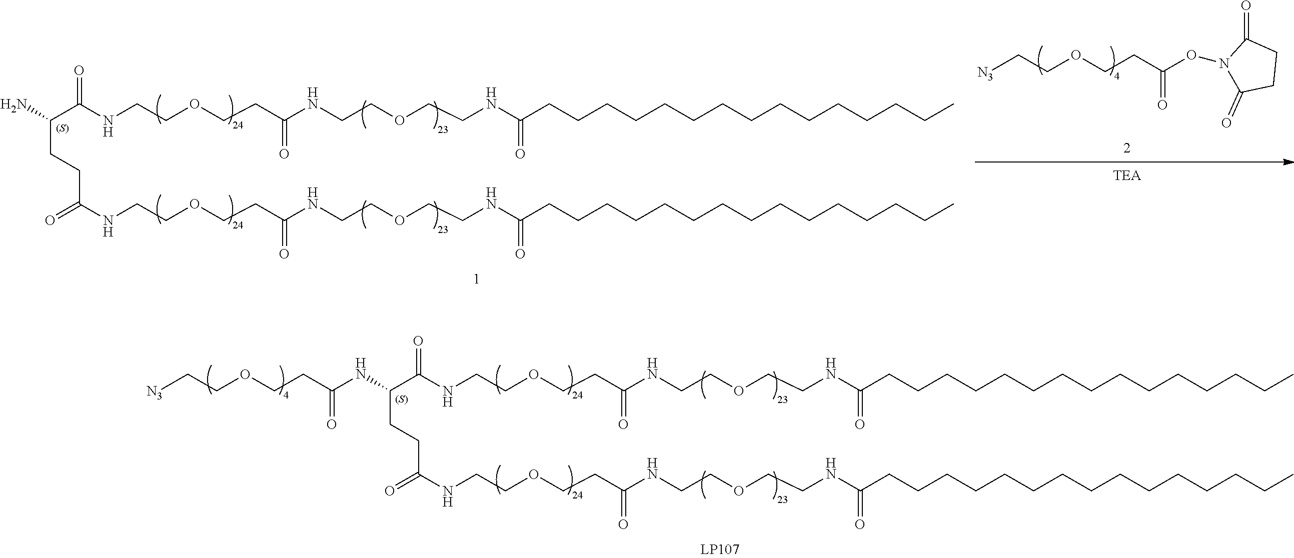

Compound 1 (982 mg, synthesized as shown in the procedures for LP38, above) was dissolved in 10 mL DCM. Compound 4 (90 mg) and triethylamine (0.081 mL) were added. The reaction was stirred at room temperature for 5-8 hours until completion. The product was extracted using 1N HCl, followed by sat. NaHCO₃ then washed brine, and finally dried with Na₂SO₄. The product was further purified using column chromatography.

### Synthesis of LP108-p

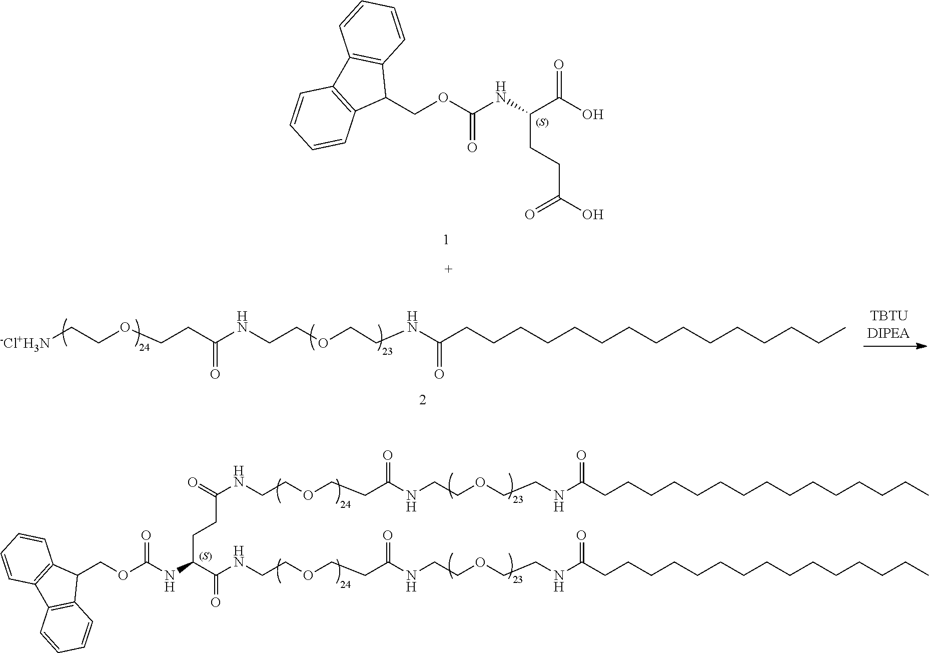

To a solution of compound 1 (595 mg, 1.610 mmol, 1.0 equiv.), compound 2 (8377 mg, 3.382 mmol, 2.10 equiv.) and diisopropylethylamine (1.122 mL, 6.443 mmol, 4.0 equiv.) in anhydrous DMF (100 mL) was added TBTU (1241 mg, 3.865 mmol, 2.4 equiv.) at room temperature. The reaction was kept at room temperature for 3 hrs. The reaction mixture was concentrated. The residue was washed with saturated ammonium chloride and sodium bicarbonate aqeuous solution. The product was purified by CombiFlash and was eluted with 12-20% methanol in dichloromethane. LC-MS: [M+5H]/5, calculated 1043.05, found 1044.38.

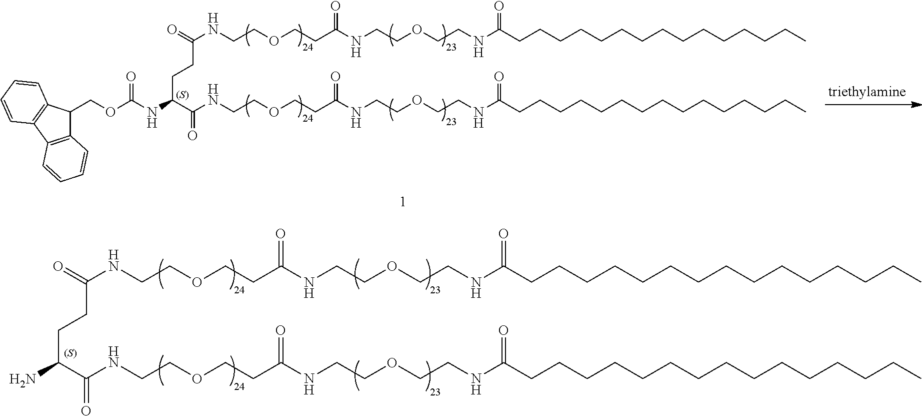

To a solution of compound 1 (104 mg, 0.0199 mmol, 1.0 equiv.) in anhydrous DMF (1.6 mL) was added triethylamine (0.4 mL) at room temperature. The reaction was kept at room temperature overnight and the solvent was concentrated. The product was used directly without further purification. LC-MS: [M+5H]/5 calculated 998.63, found 999.97.

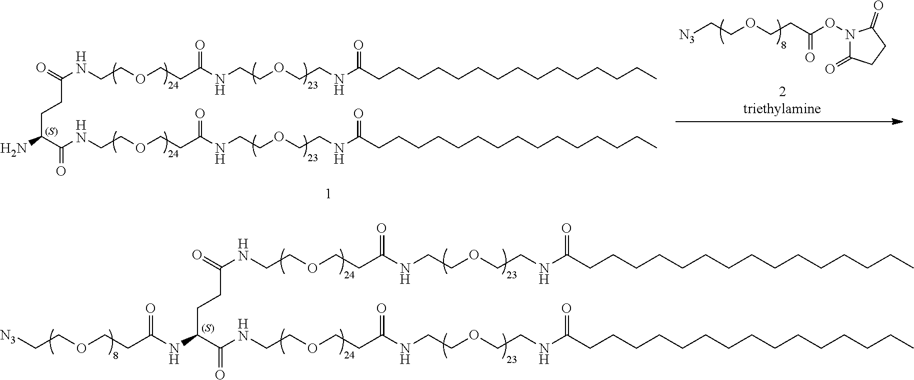

To a solution of compound 1 (99 mg, 0.198 mmol, 1.0 equiv.) and compound 2 (134 mg, 0.238 mmol, 1.2 equiv.) in anhydrous DCM (3 mL) was added triethylamine (0.006 mL, 0.0397 mmol, 2.0 equiv.) at room temperature. The reaction was kept at room temperature overnight and the solvent was concentrated. The product was separated by CombiFlash and was eluted with 12-20% methanol in dicholoromethane. LC-MS: calculated [M+5H]/5 1088.48, found 1089.86.

### Synthesis of LP108-p

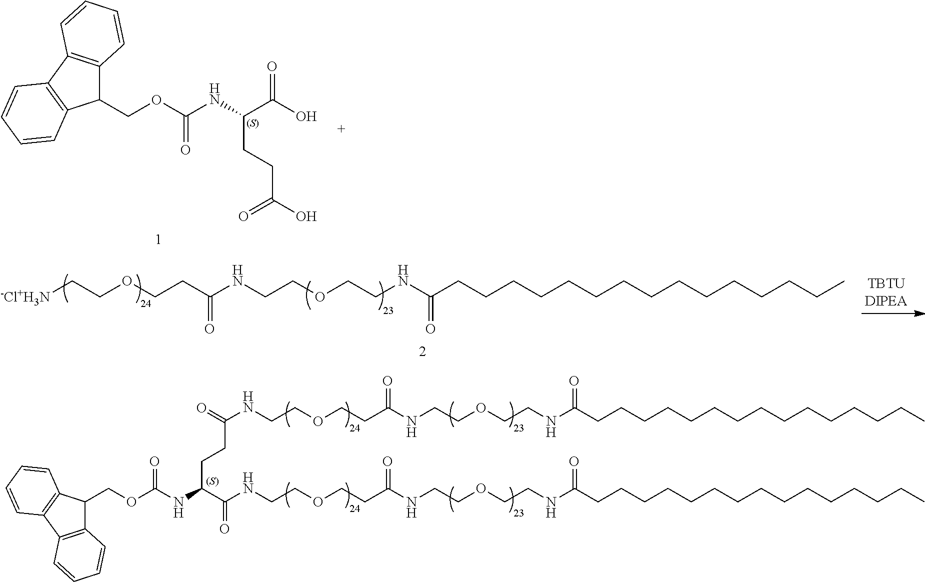

To a solution of compound 1 (595 mg, 1.610 mmol, 1.0 equiv.), compound 2 (8377 mg, 3.382 mmol, 2.10 equiv.) and diisopropylethylamine (1.122 mL, 6.443 mmol, 4.0 equiv.) in anhydrous DMF (100 mL) was added TBTU (1241 mg, 3.865 mmol, 2.4 equiv.) at room temperature. The reaction was kept at room temperature for 3 hrs. The reaction mixture was concentrated. The residue was washed with saturated ammonium chloride and sodium bicarbonate aqeuous solution. The product was purified by CombiFlash and was eluted with 12-20% methanol in dichloromethane. LC-MS: [M+5H]/5, calculated 1043.05, found 1044.38.

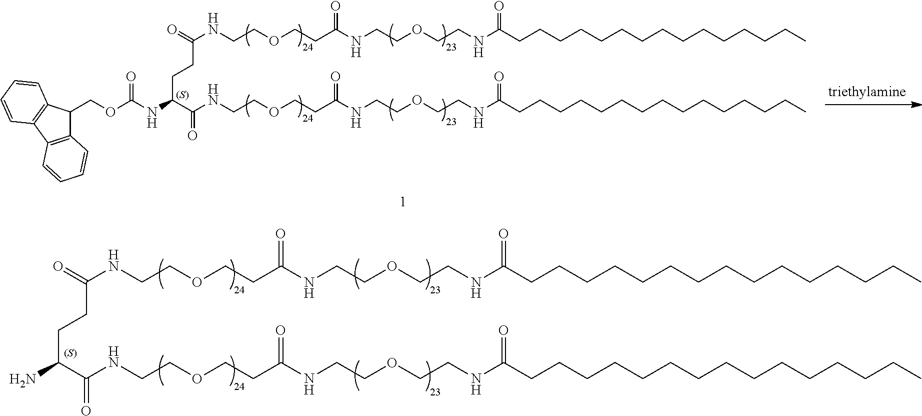

To a solution of compound 1 (104 mg, 0.0199 mmol, 1.0 equiv.) in anhydrous DMF (1.6 mL) was added triethylamine (0.4 mL) at room temperature. The reaction was kept at room temperature overnight and the solvent was concentrated. The product was used directly without further purification. LC-MS: [M+5H]/5 calculated 998.63, found 999.97.

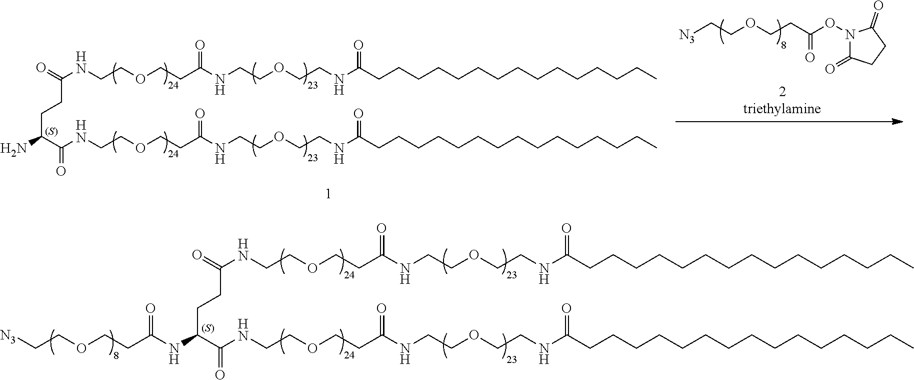

To a solution of compound 1 (99 mg, 0.198 mmol, 1.0 equiv.) and compound 2 (134 mg, 0.238 mmol, 1.2 equiv.) in anhydrous DCM (3 mL) was added triethylamine (0.006 mL, 0.0397 mmol, 2.0 equiv.) at room temperature. The reaction was kept at room temperature overnight and the solvent was concentrated. The product was separated by CombiFlash and was eluted with 12-20% methanol in dicholoromethane. LC-MS: calculated [M+5H]/5 1088.48, found 1089.86.

### Synthesis of LP109-p

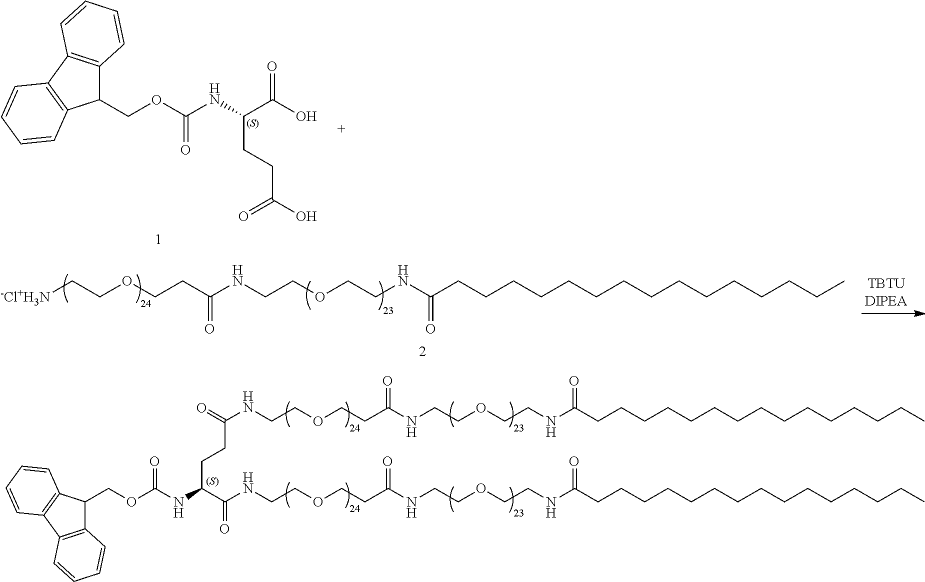

To a solution of compound 1 (595 mg, 1.610 mmol, 1.0 equiv.), compound 2 (8377 mg, 3.382 mmol, 2.10 equiv.) and diisopropylethylamine (1.122 mL, 6.443 mmol, 4.0 equiv.) in anhydrous DMF (100 mL) was added TBTU (1241 mg, 3.865 mmol, 2.4 equiv.) at room temperature. The reaction was kept at room temperature for 3 hrs. The reaction mixture was concentrated. The residue was washed with saturated ammonium chloride and sodium bicarbonate aqeuous solution. The product was purified by CombiFlash and was eluted with 12-20% methanol in dichloromethane. LC-MS: [M+5H]/5, calculated 1043.05, found 1044.38.

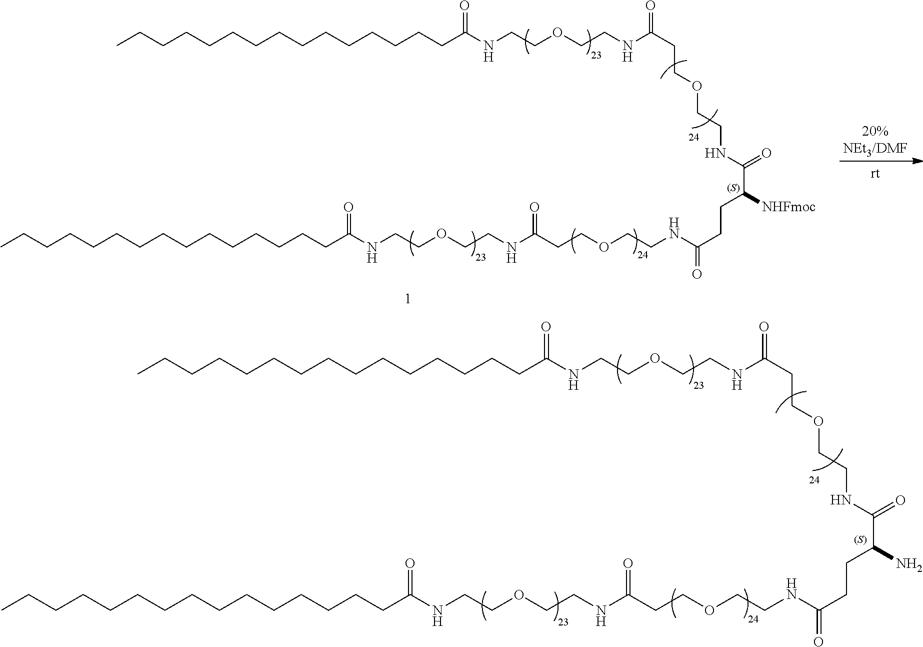

To compound 1 (100 mg) was added 20% NEt₃ (0.053 mL) in DMF at rt. The reaction was stirred under ambient conditions until full conversion was confirmed via LC-MS. The reaction mixture was azeotroped with PhMe/MeOH and concentrated under high-vacuum overnight. LC-MS: calculated [M+H]+ 4989.17 m/z, observed 1262.31 (+4/4, +H₂O) m/z.

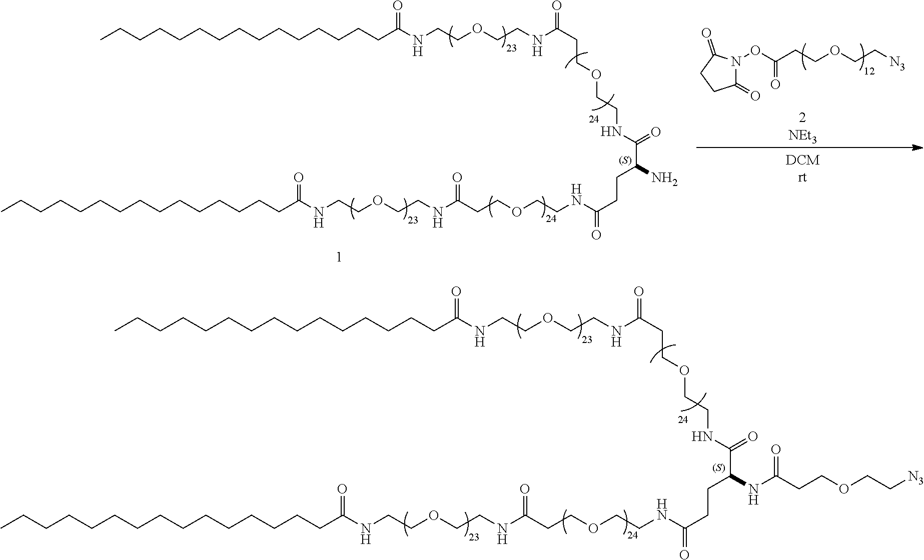

A solution of compound 1 (95.7 mg) and NEt₃ in anh. DCM (0.008 mL) under sparging N₂(g) was prepared at room temperature. Compound 2 (14.2 mg) was then added slowly. The reaction mixture was allowed to stir until full conversion was observed by LC-MS. The reaction mixture was then directly concentrated. The residue was purified by CombiFlash via a 12-g column of silica gel as the stationary phase with a gradient of DCM to 20% MeOH/DCM (0% B to 100% B) over 20 min., in which product eluted at 100% B to provide clean and impure fractions. Two clean fractions were collected and concentrated. An impure fraction was concentrated and resubjected to reaction conditions to push further conversion. Isolation via a gradient of DCM to 20% MeOH/DCM (0% B to 100% B) provided improved yet somewhat impure product elution at 88% B. LC-MS: calculated [M+H]+ 5614.51 m/z, observed 1422.64 (+4/4, +H₂O) m/z.

### Synthesis of LP110-p

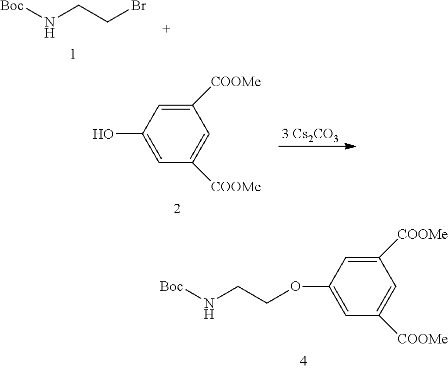 [SMILES: CC(C)(C)OC(=O)NCCBr ; COC(=O)c1cc(O)cc(C(=O)OC)c1 ; COC(=O)c1cc(OCCNC(=O)OC(C)(C)C)cc(C(=O)OC)c1]

To a solution of compound 1 (4.00 g) in 20 mL DMF was added compounds 2 (4.50 g) and 3 (11.6 g) under ambient conditions. The reaction was stirred overnight. The product was extracted by standard work up (1N NaOH, brine) and dried with Na₂SO₄ dry. TLC showed compound 2 was removed by NaOH. The product was used directly in the next step.

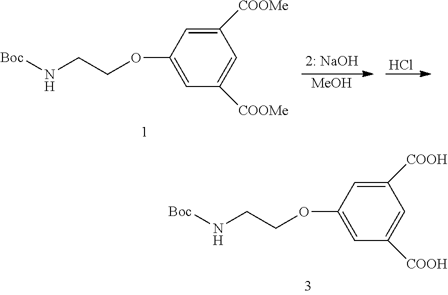 [SMILES: "MeOH" ; COC(=O)c1cc(OCCNC(=O)OC(C)(C)C)cc(C(=O)OC)c1 ; Cl ; CC(C)(C)OC(=O)NCCOc1cc(C(=O)O)cc(C(=O)O)c1]

To a solution of compound 1 (3.04 g) in 100 mL MeOH was added NaOH (1.03 g) solution under ambient conditions. The reaction was stirred overnight. The reaction mixture was concentrated to remove MeOH. The aqueous phase was extracted with ethyl acetate to remove any unreacted starting material. The mixture was acidified to pH=3, then extracted with ethyl acetate, dried using Na₂SO₄ and concentrated to produce a white solid. The product was used directly in the next step.

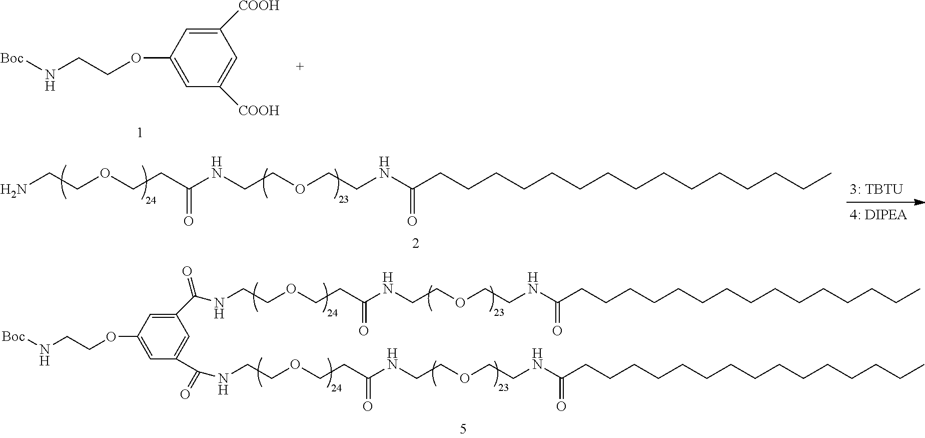

To compound 1 (2.9 mg) in DCM was added 2 equivalents of DIPEA (0.006 mL) at room temperature. Compounds 2 (45 mg), 3 (6.3 mg), and 2 equivalents of DIPEA (0.006 mL) was stirred at room temperature for 30 min. Slow addition of above activated acid mixture to PEG solution was achieved by using syringe pump (in 2-3 hours). The reaction mixture was stirred at rt. until full conversion was observed by TLC.

The product was extracted using a standard work up (1N HCl, sat. NaHCO₃, brine). The residue was purified by CombiFlash using silica gel as the stationary phase with a gradient of DCM to 20% MeOH in DCM (0-100% B).

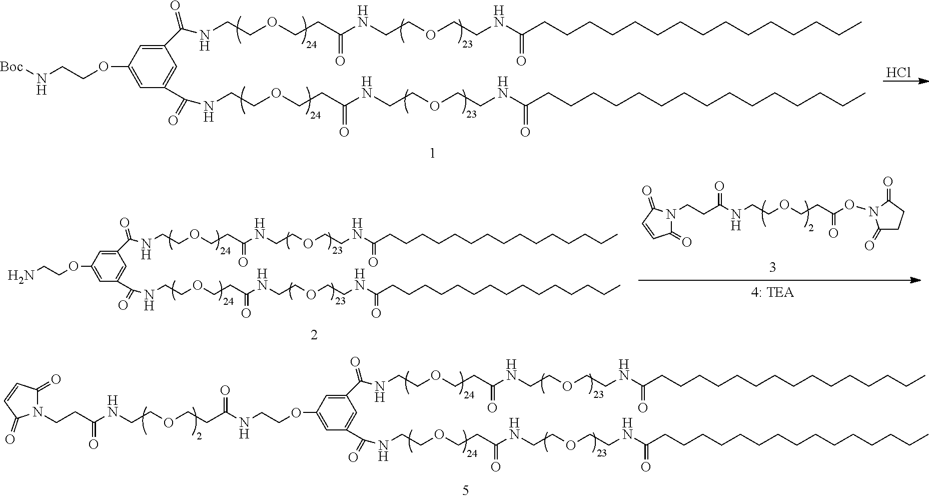

To compound 1 (27 mg) was added 1.5 mL 4 M HCl/dioxane at room temperature. The reaction was stirred under ambient conditions. The reaction was stirred for 1.5 h until full conversion was confirmed via LC-MS. The reaction concentrated under vacuum. The residue was dissolved in DCM, and compounds 3 (2.7 mg) and 4 (1.1 mg) were added. The reaction mixture was stirred at room temperature until full conversion was observed by TLC.

The reaction mixture was purified by CombiFlash using silica gel as the stationary phase with a gradient of DCM to 20% MeOH in DCM (0-100% B).

### Synthesis of LP111-p-

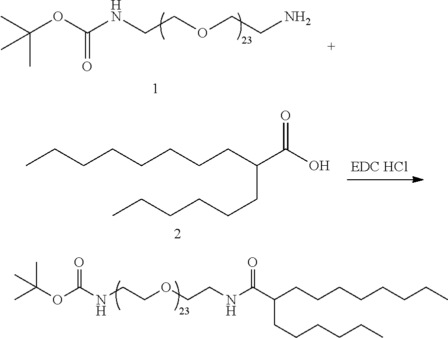

To a solution of compound 1 (2500 mg, 2.130 mmol, 1.0 equiv.) and compound 2 (655 mg, 2.556 mmol, 1.2 equiv.) in anhydrous DCM (10 mL) was added EDC HCl (630 mg, 3.195 mmol, 1.5 equiv.) at room temperature. The reaction was kept at room temperature overnight. The reaction mixture was concentrated. The product was purified by CombiFlash and was eluted with 8-18% methanol in dichloromethane. LC-MS: calculated [M+H]+ 1411.95, found 1412.80.

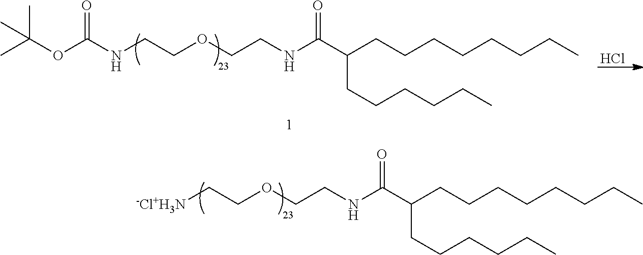

To compound 1 (2400 mg, 1.699 mmol, 1.0 equiv.) was added 4M HCl in dioxane (8.499 mL, 33.997 mmol, 20 equiv.) at room temperature. The reaction was kept at room temperature for 1 hr. The reaction mixture was concentrated. The product was used directly without further purification. LC-MS: [M+H]/+ calculated 1311.90, found 1312.95.

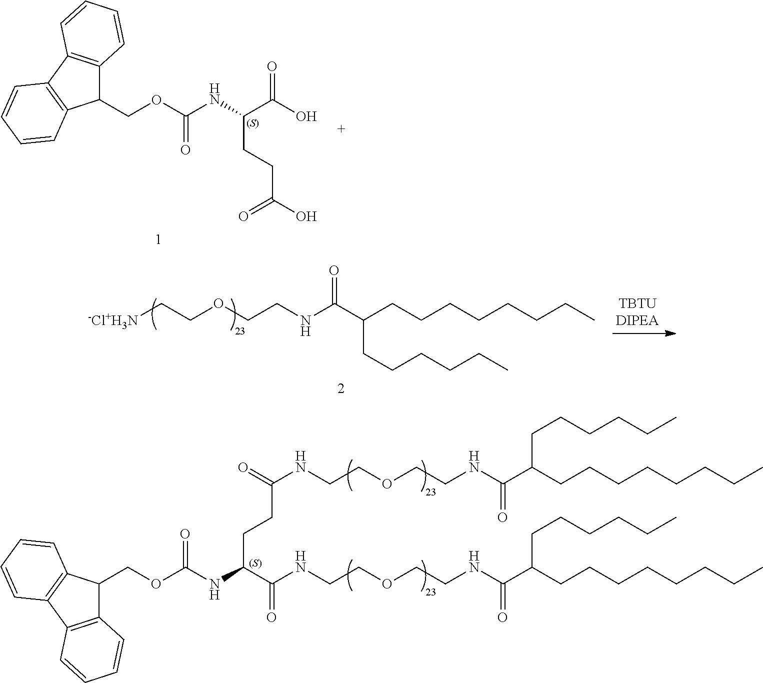

To a solution of compound 1 (300 mg, 0.812 mmol, 1.0 equiv.), compound 2 (2.299 g, 1.705 mmol, 2.10 equiv.) and diisopropylethylamine (0.566 mL, 3.248 mmol, 4.0 equiv.) in anhydrous DMF (10 mL) was added TBTU (625 mg, 1.949 mmol, 2.4 equiv.) at room temperature. The reaction was kept at room temperature for 1 hr. The reaction mixture was concentrated. The residue was washed with saturated ammonium chloride and sodium bicarbonate aqeuous solution. The product was purified by CombiFlash and was eluted with 10-18% methanol in dichloromethane. LC-MS: [M+2H]/2, calculated 1478.45, found 1479.89.

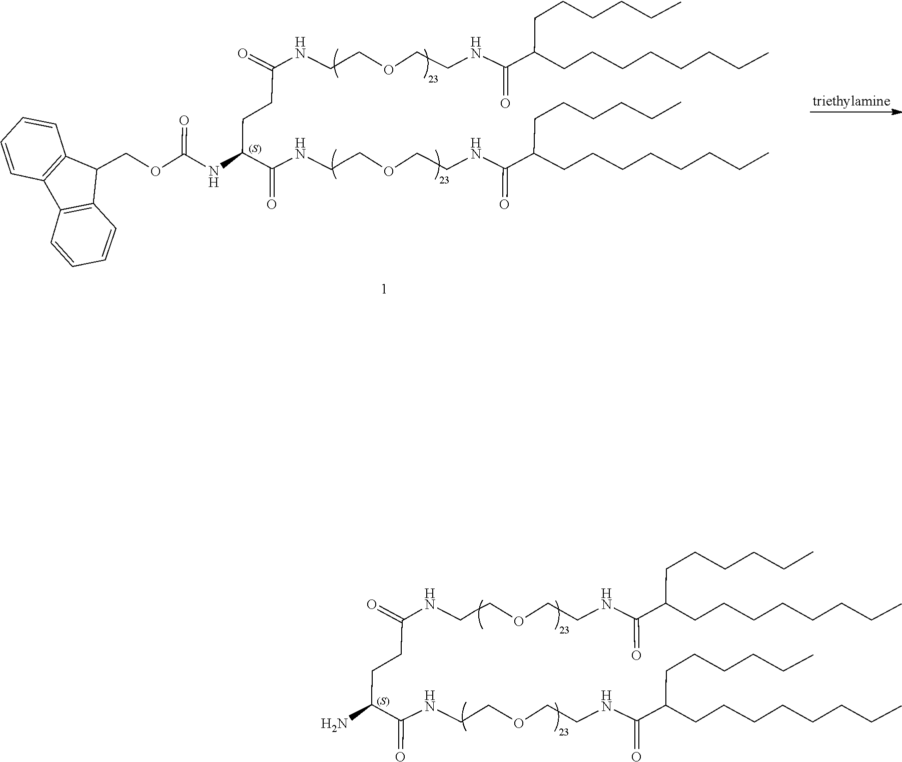

To a solution of compound 1 (1690 mg, 0.571 mmol, 1.0 equiv.) in anhydrous DMF (8 mL) was added triethylamine (2 mL) at room temperature. The reaction was kept at room temperature overnight and the solvent was concentrated. The product was used directly without further purification. LC-MS: [M+2H]/2 calculated 1367.41, found 1368.88.

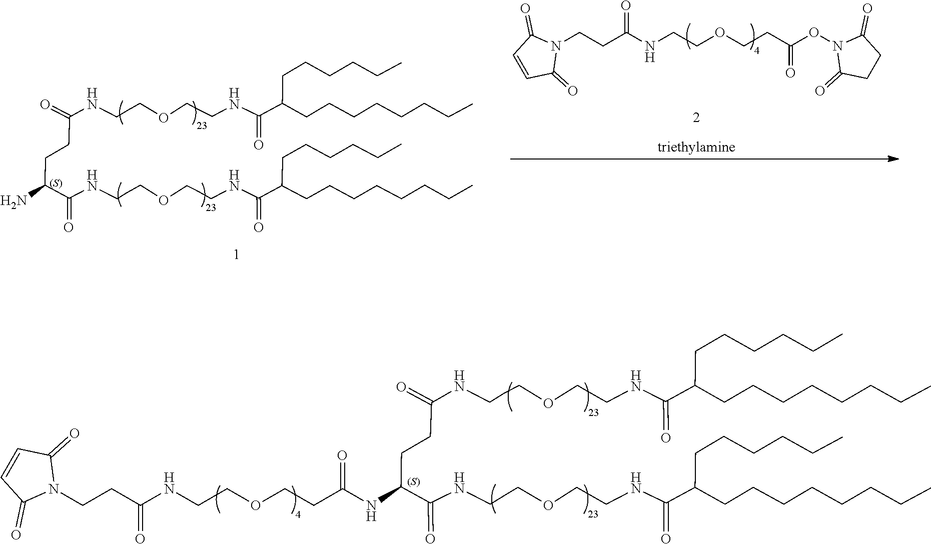

To a solution of compound 1 (1563 mg, 0.571 mmol, 1.0 equiv.) and compound 2 (381 mg, 0.743 mmol, 1.3 equiv.) in anhydrous DCM (10 mL) was added triethylamine (0.162 mL, 1.143 mmol, 2.0 equiv.) at room temperature. The reaction was kept at room temperature overnight and the solvent was concentrated. The product was separated by CombiFlash and was eluted with 8-16% methanol in dicholoromethane. LC-MS: calculated [M+3H]/3 1044.67, found 1046.18.

### Synthesis of LP124-p

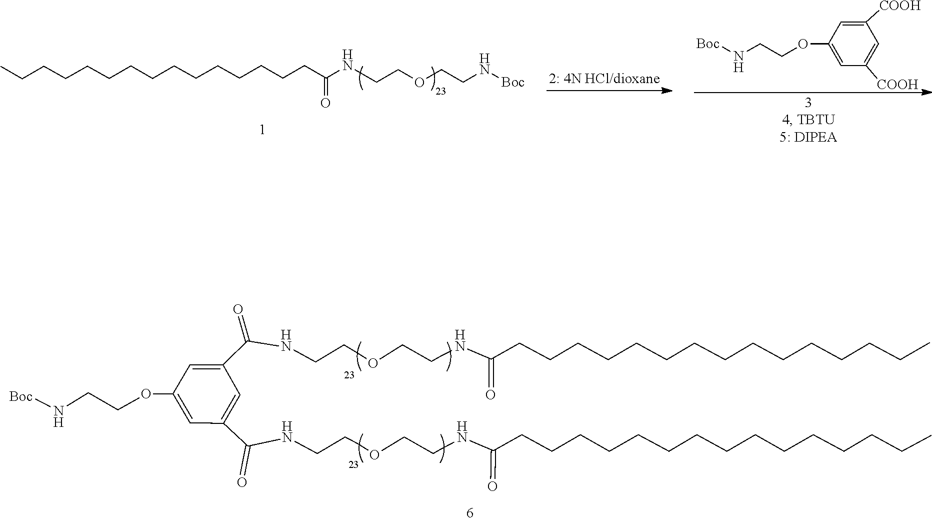

To compound 1 (760 mg) was added 2 mL of 4 M HCl/dioxane at room temperature. The reaction was stirred under ambient conditions. The reaction was stirred for 1.5 h until full conversion was confirmed via LC-MS. The reaction concentrated under vacuum. The residue was dissolved in DCM, and compounds 3 (84.1 mg), 4 (207 mg) and 5 (0.281 mL) were added. The reaction mixture was stirred at room temperature until full conversion was observed by TLC.

The product was extracted by standard work up (1N HCl, sat. NaHCO₃, brine). The residue purified by CombiFlash using silica gel as the stationary phase with a gradient of DCM to 20% MeOH in DCM (0-100% B).

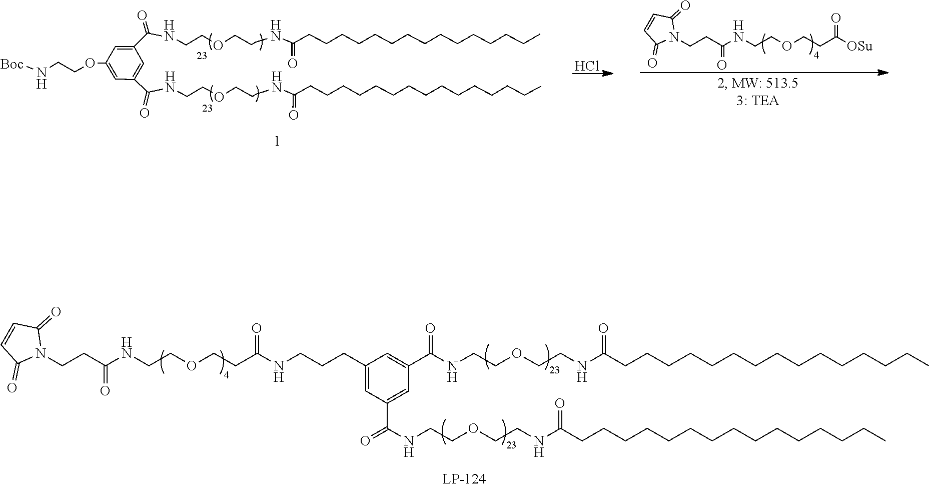

To compound 1 (250 mg) was added 4 mL 4 M HCl/dioxane at room temperature. The reaction was stirred under ambient conditions for 2 h until full conversion was confirmed via LC-MS. The reaction was concentrated under vacuum. The residue was dissolved in DCM, then compounds 2 (52.9 mg) and 3 (0.036 mL) were added. The reaction mixture was stirred at room temperature until full conversion was observed by TLC.

The reaction mixture was purified by CombiFlash using silica gel as the stationary phase with a gradient of DCM to 20% MeOH in DCM (0-100% B).

### Synthesis of LP130-p

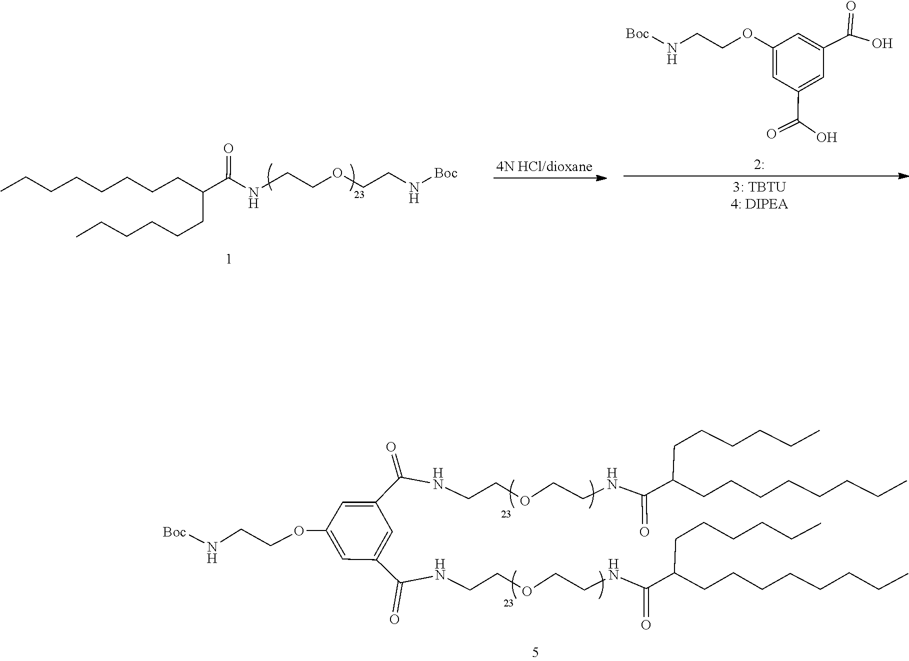

To compound 1 (1.89 g) was added 5 mL of 4 M HCl/dioxane at room temperature. The reaction was stirred under ambient conditions for 1.5 h until full conversion was confirmed via LC-MS. The reaction was concentrated under vacuum. The residue was dissolved in DCM, and compounds 2 (209 mg), 3 (516 mg) and 4 (0.70 mL) were added. The reaction mixture was stirred at room temperature until full conversion was observed by TLC.

The product was extracted by a standard work up (1N HCl, sat. NaHCO₃, brine). The residue was purified by CombiFlash using silica gel as the stationary phase with a gradient of DCM to 20% MeOH in DCM (0-100% B).

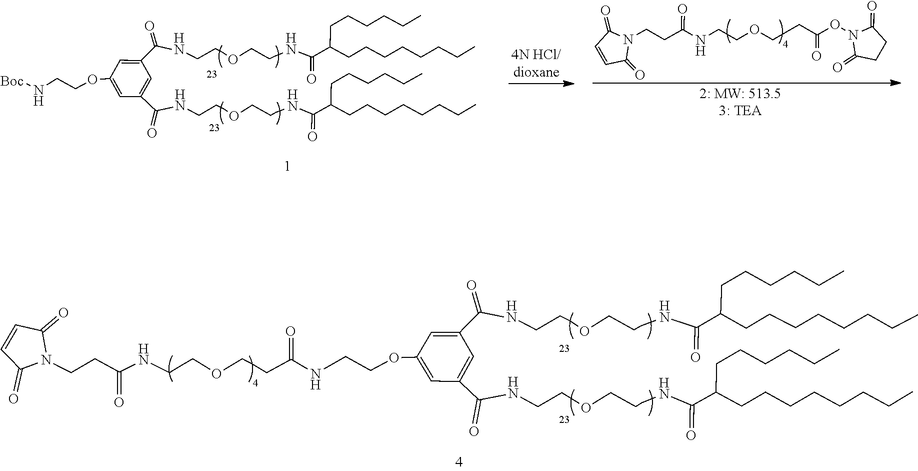

To compound 1 (800 mg) was added 5 mL of 4 N HCl/dioxane at room temperature. The reaction was stirred under ambient conditions for 2 h until full conversion was confirmed via LC-MS. The reaction was concentrated under vacuum. The residue was dissolved in DCM, then compounds 2 (169 mg) and 3 (0.116 mL) were added. The reaction mixture was stirred at room temperature until full conversion was observed by TLC.

The reaction mixture was purified by CombiFlash using silica gel as the stationary phase with a gradient of DCM to 20% MeOH in DCM (0-100% B).

### Synthesis of LP143-p

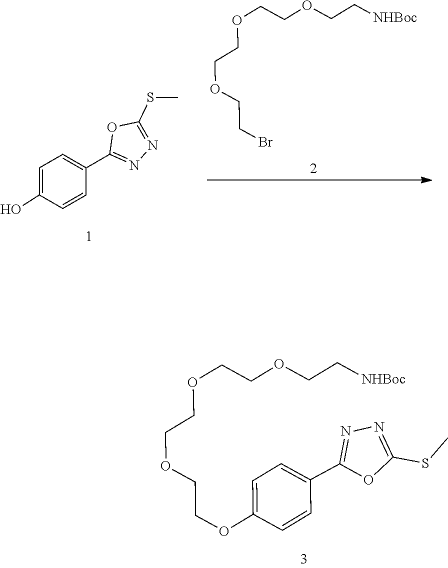

Compound 1 (500 mg) was dissolved in 10 mL anhydrous THE in a pressure vessel and K₂CO₃ (398 mg) was added. Compound 2 (983 mg) was added as a solution in a minimal amount of DMF and the vessel was capped and the reaction was set to stir overnight at 40° C. Then, the reaction was allowed to cool to room temperature. The solids were filtered off and the reaction was concentrated under vacuum. FCC 0-100% EA in hexanes.

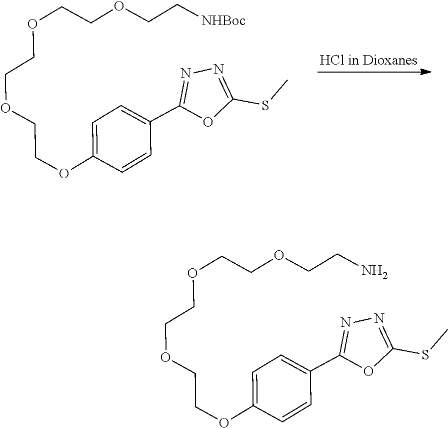 [SMILES: CSc1nnc(-c2ccc(OCCOCCOCCOCCNC(=O)OC(C)(C)C)cc2)o1 ; CSc1nnc(-c2ccc(OCCOCCOCCOCCN)cc2)o1]

Compound 1 (1070 mg) was dissolved in 4 mL of 4 M HCl in dioxanes and stirred until all Boc was removed. The reaction was then concentrated. FCC 0-20% MeOH in DCM.

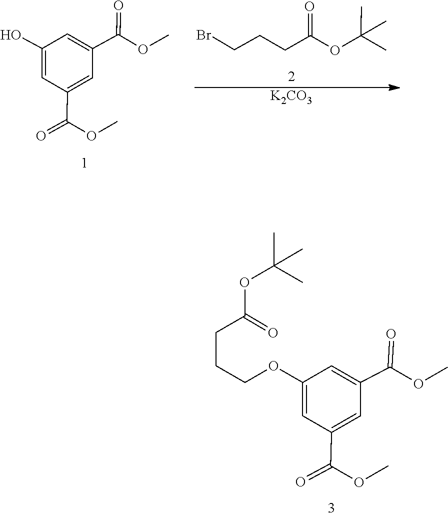

Compound 1 (1000 mg) was dissolved in 5 mL anhydrous DMF in a pressure vessel and K₂CO₃ (1.315 g) was added. Then compound 2 (850 mg) was added in a minimal amount of DMF and the reaction was capped and stirred at 40° C. Then, the reaction was allowed to cool to room temperature. The solids were filtered off and then the reaction was concentrated under vacuum. FCC 0-100% EA in hexanes.

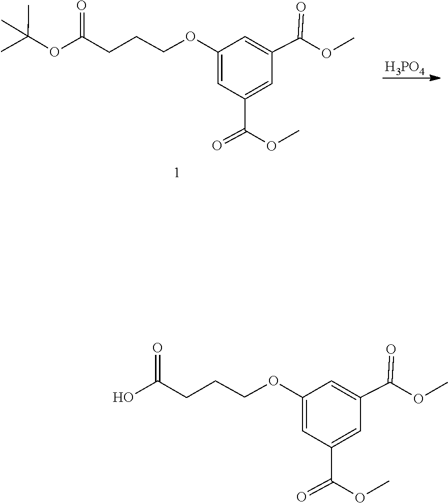

H₃PO₄ (0.594 mL) was added to a stirring solution of compound 1 (900 mg) in 20 mL of toluene. The reaction was stirred overnight at room temperature. The reaction was then diluted with water (30 mL) and washed 3× with ethyl acetate (30 mL). The organic layers were dry-pooled over sodium sulfate and concentrated.

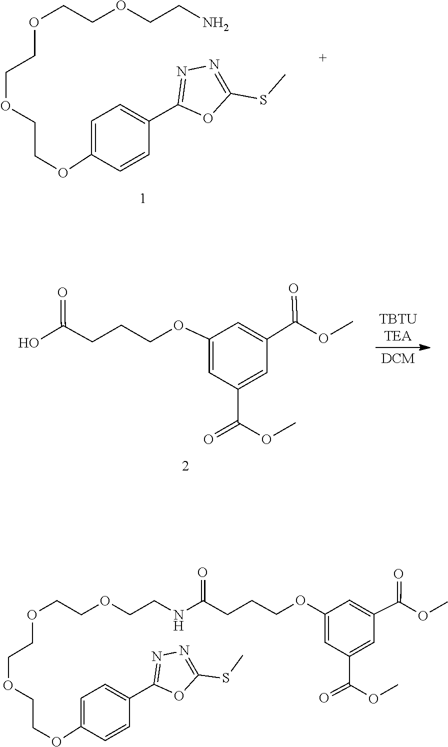 [SMILES: CSc1nnc(-c2ccc(OCCOCCOCCOCCN)cc2)o1 ; COC(=O)c1cc(OCCCC(=O)O)cc(C(=O)OC)c1 ; COC(=O)c1cc(OCCCC(=O)NCCOCCOCCOCCOc2ccc(-c3nnc(SC)o3)cc2)cc(C(=O)OC)c1]

Compound 2 (100 mg) and TBTU (149 mg) were dissolved in 2 mL DMF and were stirred for 5 min. Then TEA (0.152 mL) and compound 1 (142 mg) were added to the mixture and the reaction was stirred at room temperature overnight. The reaction was diluted with ethyl acetate (10 mL) and washed with saturated ammonium chloride (3×10 mL). The organic layer was dried over sodium sulfate and concentrated. FCC 0-100% hex-EA and then swap to DCM/MeOH 0-20%.

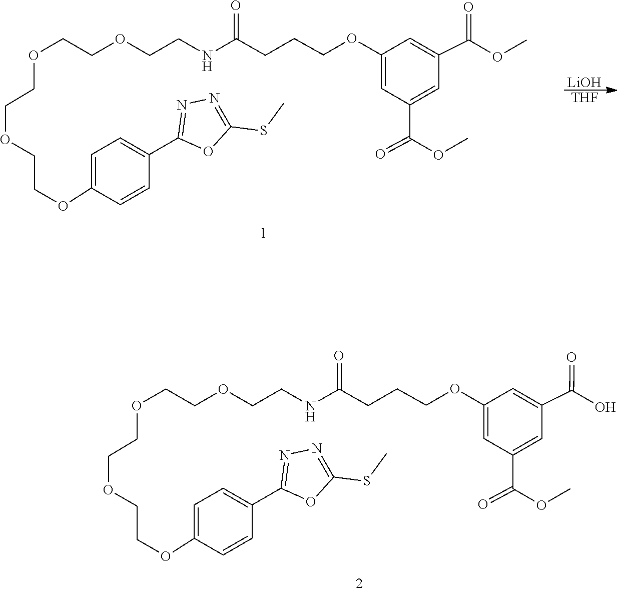 [SMILES: COC(=O)c1cc(OCCCC(=O)NCCOCCOCCOCCOc2ccc(-c3nnc(SC)o3)cc2)cc(C(=O)OC)c1 ; "LiOH" ; COC(=O)c1cc(OCCCC(=O)NCCOCCOCCOCCOc2ccc(-c3nnc(SC)o3)cc2)cc(C(=O)O)c1 ; "THF"]

Compound 1 (197 mg) was dissolved in 4 mL THF. Then LiOH (43 mg) and water (0.4 mL) were added. The reaction was stirred until deprotection was confirmed by LC-MS. The reaction was quenched with amberlyst 15. The Amberlyst was filtered off and the reaction was concentrated. FCC 0-100% EA in hex with 0.1% HOAc additive.

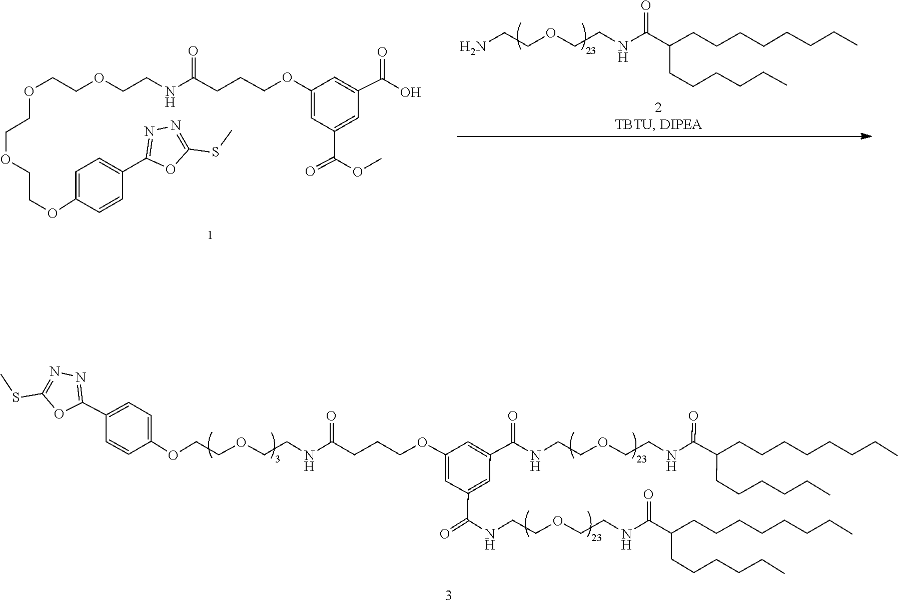

Compound 1 (380 mg) was mixed with TBTU (424 mg) in 4 mL DMF for five minutes. Then compound 2 (2.12 g) was added, followed by DIPEA (0.542 mL). The reaction was stirred at room temperature and kickers were added as follows: 50% TBTU and 50% DIPEA at 2 hrs., 25% TBTU and 50% DIPEA at 3 hrs., 50% DIPEA at 4 hrs., 50% DIPEA at hrs. The reaction was quenched after 6.5 hours. The reaction was diluted with 20% TFE in DCM (15 mL) and washed with saturated ammonium chloride 2× (15 mL). The organic layer was dried over sodium sulfate and concentrated then purified by HPLC.

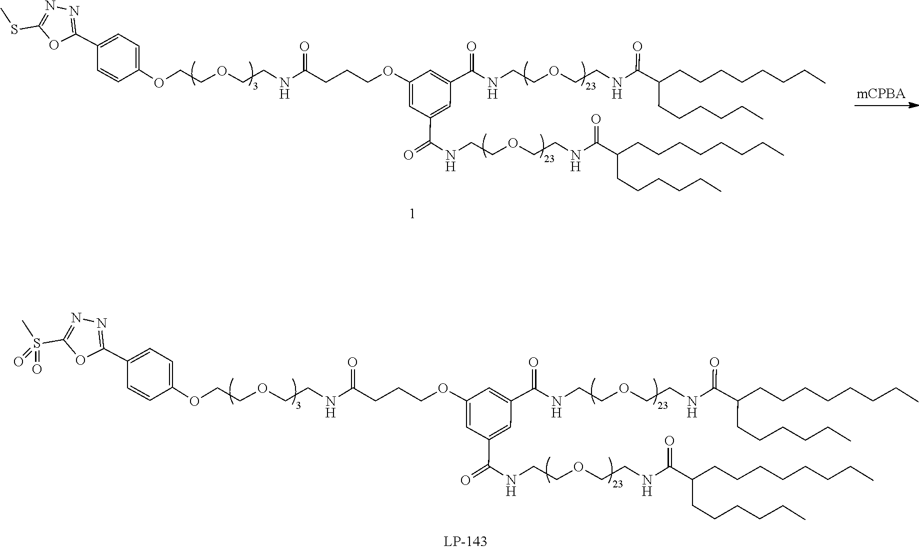

mCPBA (70% pure, 12 mg) was added to a stirring solution of compound 1 (28 mg) in 1 mL DCM at 0° C. The mixture was allowed to warm up to room temperature stirring overnight and monitored via LCMS. The mixture was diluted with 20% TFE in DCM (5 mL), then washed with saturated sodium sulfite (2×5 mL) and once with saturated sodium bicarbonate (5 mL). The organic layer was dried over sodium sulfate. Correct mass was confirmed by LC-MS.

### Synthesis of LP210-p

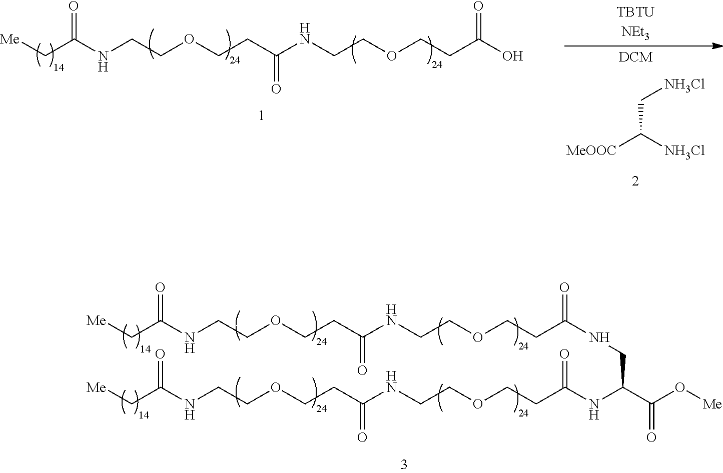

Compound 1 (0.2 g, 0.08 mmol) and TBTU (0.0542 g, 0.735 mmol) were dissolved in DCM (5 mL) and NEt₃ (0.0244 mL, 0.175 mmol) was added. In a separate vial, compound 2 (0.007 g, 0.037 mmol) and NEt₃ (0.0244 mL, 0.175 mmol) were stirred together in DCM (1 mL). The resulting solutions were stirred for 10 minutes. After 10 minutes the solution of compound 2 was added to the solution of compound 1. The resulting mixture was stirred for 90 minutes and then checked by LC-MS. The reaction mixture was quenched with 5 mL of water and stirred for 5 minutes. The layers were separated, and the organic layer was washed with sat. NaHCO₃(aq) (2×20 mL), water (20 mL), sat. NH₄C₁(aq) (2×20 mL), sat. NaCl(aq) (2×20 mL), dried over Na₂SO₄ and concentrated to yield crude compound 3 as a waxy off white solid (ca. 200 mg). The crude product was purified by silica gel chromatography eluting with 0-20% MeOH in DCM. Pure fractions were combined to yield 50 (27% yield) of compound 3 as a white solid.

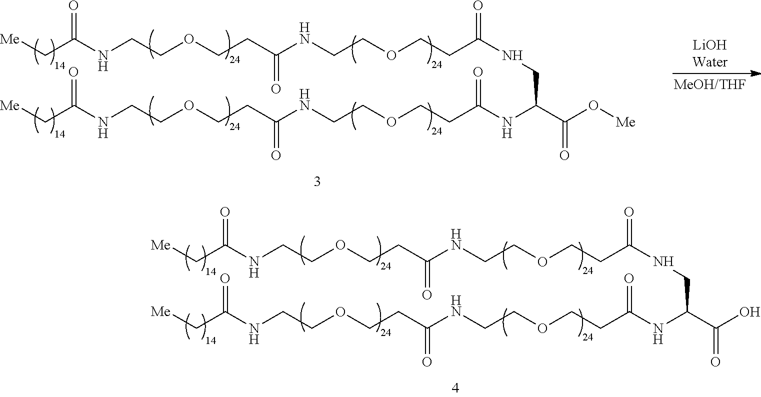

Compound 3 (0.05 g, 0.010 mmol) was dissolved in 1:1 MeOH/THF (5 mL), and LiOH (0.042 g, 1.74 mmol) and water (100 μL, 5.55 mmol) was added. The reaction mixture was stirred at room temperature overnight and checked by LC-MS. Organics were evaporated off and the resulting suspension was diluted with approximately 10 mL of water. The resulting suspension was acidified with 3 M HCl(aq) to a pH of 1 and was extracted with DCM (3×25 mL) The combined organics were washed with brine, dried over Na₂SO₄, concentrated, and dried under vacuum to yield 49 mg (98% yield) of compound 4 as an off white solid. The product was used without further purification.

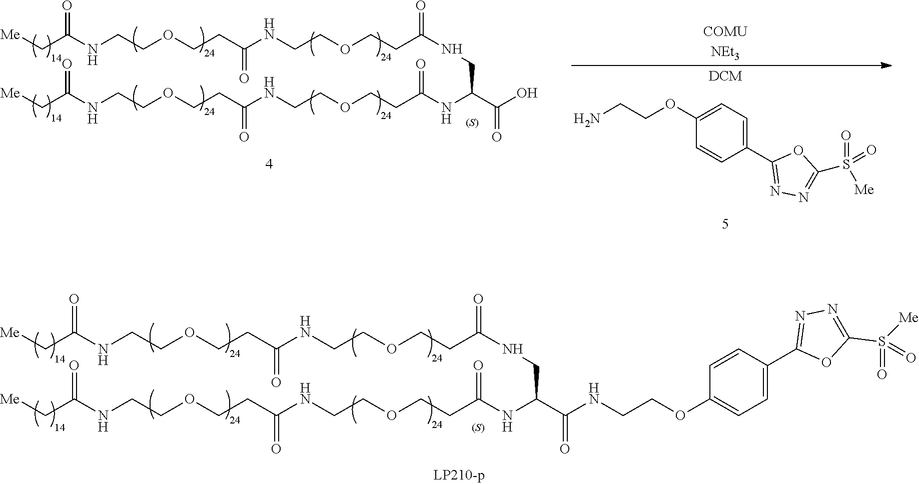

Compound 4 (0.05 g, 0.010 mmol) and COMU (0.0063 g, 0.015 mmol) were dissolved in DCM (1 mL) and NEt₃ (13.7 μL, 0.098 mmol) was added, the resulting solution was stirred for 10 minutes. In a separate vial Compound 5 was dissolved in DCM (0.3 mL). After 10 minutes the solution of compound 5 was added to the solution containing 1807-019. The resulting solution was stirred for 2 hrs. The reaction was quenched with 1 M HCl(aq) (10 mL) and the organic layer was diluted with 10 mL DCM. The layers were separated, and the organic layer was further washed with 1M HCl(aq) (20 mL), sat. NHCO3(aq) (1×20 mL) sat. NaCl(aq) (1×20 mL), dried over Na₂SO₄, concentrated, and dried under vacuum to yield 94 mg of crude LP210-p as an off white solid. The crude product was purified by silica gel chromatography eluting with 0-20% MeOH in DCM. Fractions containing pure LP210-p were combined and concentrated to yield 7 mg (13.3% yield).

### Synthesis of LP217-p

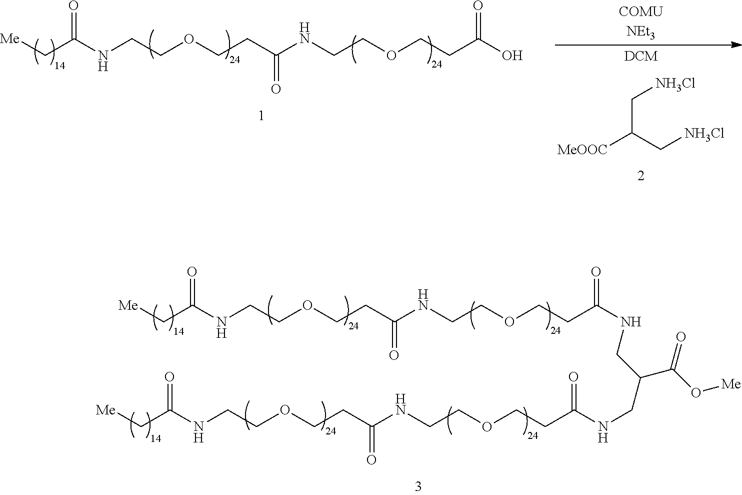

Compound 1 (0.265 g, 0.105 mmol) and COMU (0.0542 g, 0.735 mmol) were dissolved in DCM (5 mL) and NEt₃ (0.1 mL, 0.74 mmol) was added. The resulting solution was stirred for 10 minutes. After 10 minutes, compound 2 (0.010 g, 0.049 mmol) was added to the reaction. The resulting mixture was stirred overnight and checked by LC-MS. The reaction mixture was quenched with 5 mL of water and stirred for 5 minutes. The layers were separated, and the organic layer was washed with sat. NaHCO₃(aq) (2×20 mL), Water (20 mL), 2 M HCl(aq) (2×20 mL), sat. NaCl(aq) (20 mL), dried over Na₂SO₄, and concentrated to yield crude compound 3 as a waxy off white solid (ca. 350 mg). Crude compound 3 was purified by silica gel chromatography 2-20% MeOH in DCM. Fractions containing compound 3 were combined to yield 89 mg (36% yield) as an off white solid.

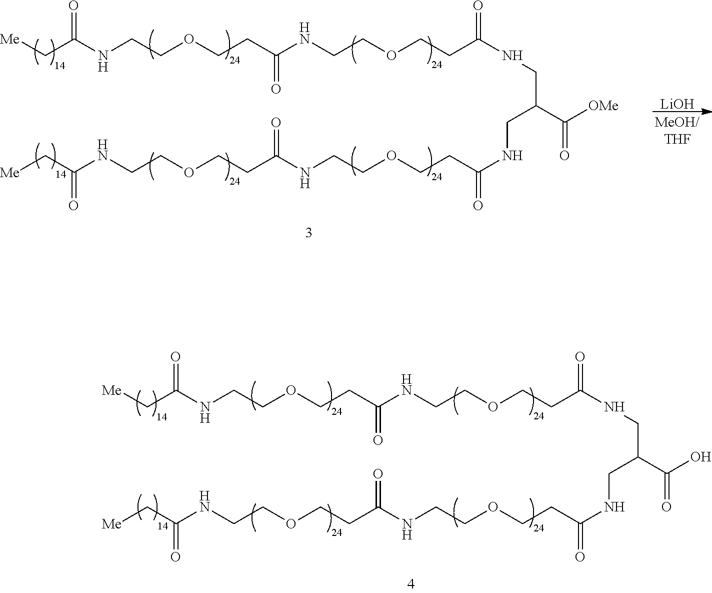

Compound 3 (0.089 g, 0.017 mmol) was dissolved in 1:1 MeOH/THF (5 mL) and LiOH (0.042 g, 1.74 mmol) and water (180 μL, 9.85 mmol) was added. The reaction mixture was stirred at room temperature overnight and checked by LC-MS. Organics were evaporated off and the resulting suspension was diluted with approximately 10 mL of water. The suspension was acidified with 3 M HCl(aq) to a pH of 1 and was extracted with DCM (3×25 mL). The combined organic layers were washed with brine, dried over Na₂SO₄, concentrated, and dried under vacuum to yield 81 mg (91% yield) of compound 4 as an off white solid. The product was used without further purification.

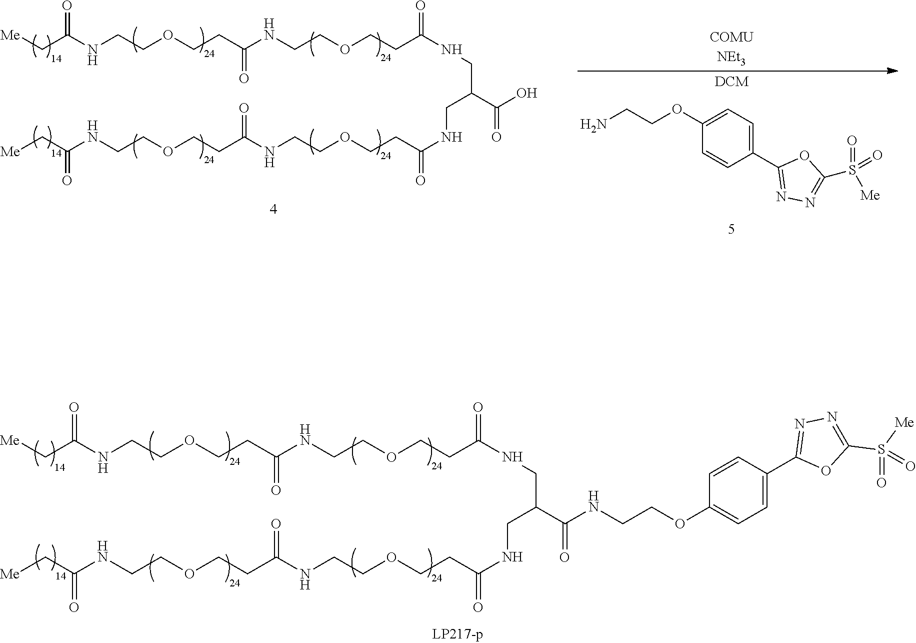

Compound 4 (0.081 g, 0.016 mmol) and COMU (0.010 g, 0.024 mmol) were dissolved in DCM (1 mL) and NEt₃ (44.2 μL, 0.32 mmol) was added. The resulting solution was stirred for 10 minutes. In a separate vial, compound 5 was dissolved in DCM (0.3 mL). After 10 minutes, the solution of compound 5 was added to the solution containing compound 4. The resulting mixture was stirred for 2 hours. The reaction was quenched with 1 M HCl(aq) (10 mL) and the organic later was diluted with 10 mL DCM. The layers were separated, and the organic layer was further washed with 1M HCl(aq) (20 mL), sat. NHCO₃(aq) (1×20 mL) sat. NaCl(aq) (1×20 mL), dried over Na₂SO₄, concentrated, and dried under vacuum to yield 94 mg of crude LP217-p as an off white solid. The crude product was purified by silica gel chromatography 0-20% MeOH in DCM. Fractions containing pure LP217-p were combined and concentrated to yield 24 mg (28% yield).

### Synthesis of LP220-p

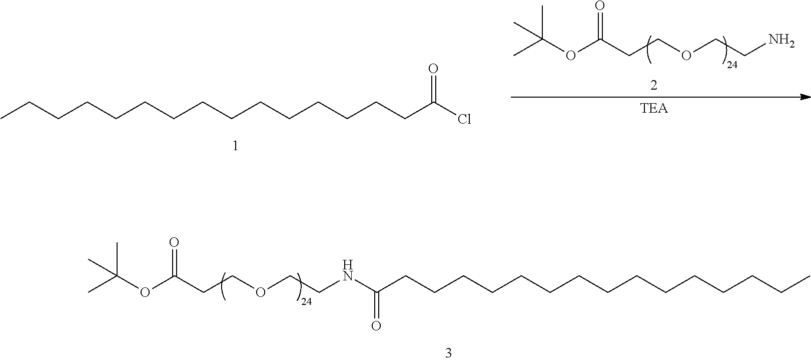

To a solution of compound 2 (3.3381 mmol, 4.0140 g) and TEA (4.0058 mmol, 0.4054 g, 0.558 mL) in DCM was added compound 1 (3.5050 mmol, 0.9634 g, 1.059 mL). The reaction mixture was stirred until full conversion of compound 2 was observed by LC-MS. The residue was purified by standard work up (1N HCl, sat. NaHCO₃, Brine wash, and dried over Na₂SO₄). Compound 3 was used without further purification. Yield: 4.5 g.

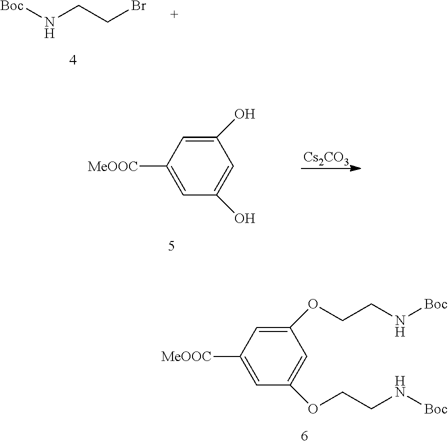

To a solution of compound 5 (29.7354 mmol, 5.0000 g) in 50 mL DMF was added compound 4 (65.4178 mmol, 13.7502 g) and Cs₂CO₃ (118.9414 mmol, 38.7535 g) at room temperature. The reaction mixture was stirred at 60° C. overnight. The reaction mixture was purified by standard work up (1N NaOH, Brine wash, and dried over Na₂SO₄). Compound 6 was purified by silica gel chromatography and concentrated to yield 6.0 g.

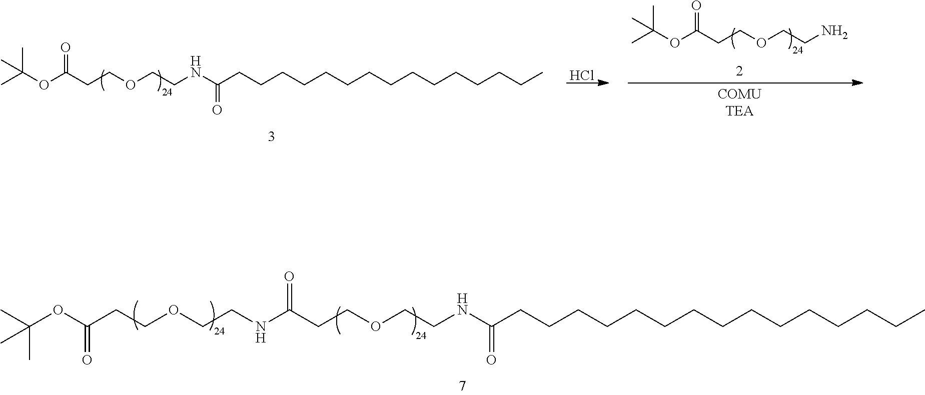

Compound 3 (1.0500 mmol, 1.5129 g) was dissolved in 8 mL 4N HCl/dioxane, and stirred at room temperature for 5 hours. After HCl was removed, compound 2 (1.0000 mmol, 1.2020 g), COMU (1.2000 mmol, 0.5139 g), and TEA (3.0000 mmol, 0.3035 g, 0.418 mL) in DCM was added. The reaction mixture was stirred until full conversion of compound 2 was observed by TLC. The residue was purified by standard work up (1N HCl, sat. NaHCO₃, Brine wash, and dried over Na₂SO₄). Compound 7 was purified by silica gel chromatography and concentrated to yield 2.28 g.

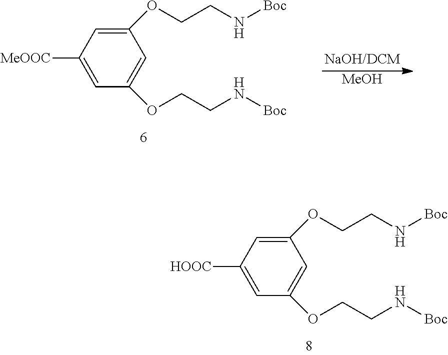 [SMILES: "MeOH" ; COC(=O)c1cc(OCCNC(=O)OC(C)(C)C)cc(OCCNC(=O)OC(C)(C)C)c1 ; CC(C)(C)OC(=O)NCCOc1cc(OCCNC(=O)OC(C)(C)C)cc(C(=O)O)c1]

To a solution of NaOH in 5 mL MeOH was added compound 6 (1.0000 mmol, 0.4545 g) in 20 mL DCM at room temperature. The reaction mixture was stirred at room temperature overnight. The reaction mixture was acidified to pH of 3. The product was dried with Na₂SO₄ to yield 0.200 g a of compound 8 that was used without further purification.

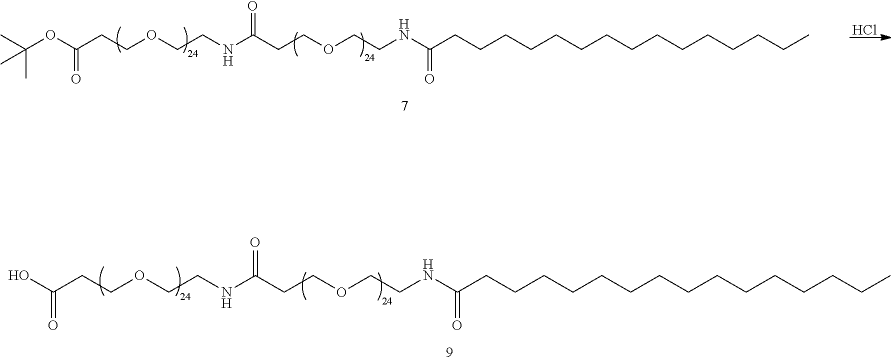

Compound 7 (0.7707 mmol, 1.9800 g) was dissolved in 10 mL 4N HCl/dioxane at room temperature overnight. The solvent was removed and the product was placed under vacuum for 2 hours to yield 1.50 g of compound 9 that was used without further purification.

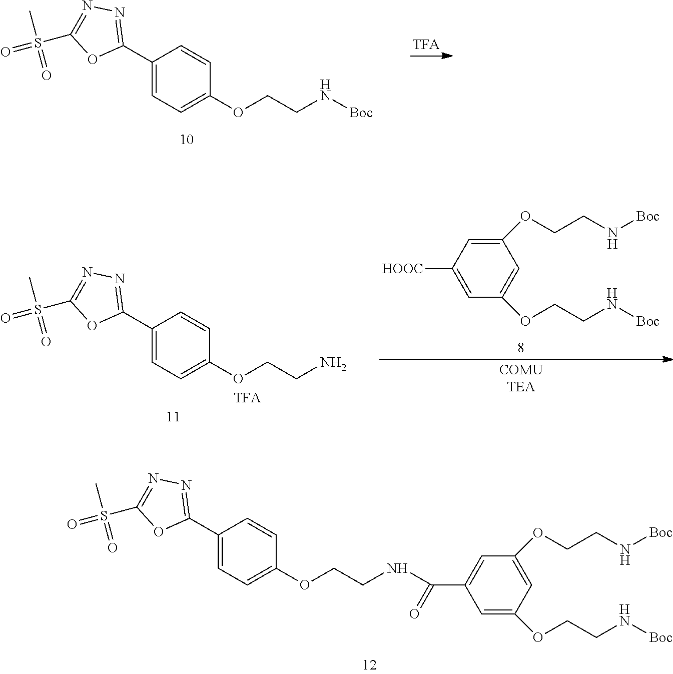 [SMILES: CC(C)(C)OC(=O)NCCOc1ccc(-c2nnc(S(C)(=O)=O)o2)cc1 ; CS(=O)(=O)c1nnc(-c2ccc(OCCN)cc2)o1 ; CC(C)(C)OC(=O)NCCOc1cc(OCCNC(=O)OC(C)(C)C)cc(C(=O)O)c1 ; CC(C)(C)OC(=O)NCCOc1cc(OCCNC(=O)OC(C)(C)C)cc(C(=O)NCCOc2ccc(-c3nnc(S(C)(=O)=O)o3)cc2)c1]

Compound 10 (0.0782 mmol, 0.0300 g) was dissolved in 1 mL DCM, and 0.5 mL TFA was added and the mixture was stirred for 2 hours. TFA was removed and compound 11 was dried under vacuum for 1 hour. Compound 8 (0.0822 mmol, 0.0362 g), COMU (0.0939 mmol, 0.0402 g), and TEA (0.2347 mmol, 0.0237 g, 0.033 mL) were dissolved in 5 mL DCM for 5 min then compound 11 in DCM was added. The reaction mixture was stirred until full conversion of compound 11 was observed by TLC. Compound 12 was purified by silica gel chromatography to yield 0.0135 g.

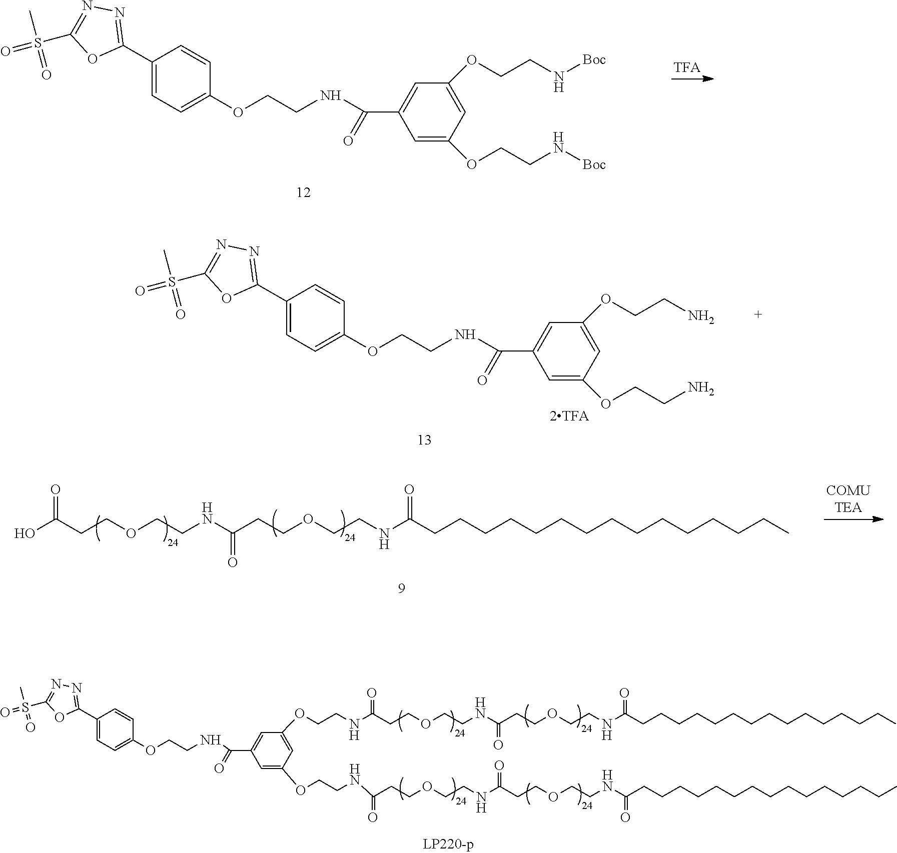

Compound 12 (0.0191 mmol, 0.0135 g) was dissolved in 1 mL DCM, 0.5 mL of TFA was added and the mixture was stirred for 1 hour. TFA was removed and compound 13 was dried under vacuum for 1 hour. Compound 9 (0.0398 mmol, 0.1000 g), COMU (0.0477 mmol, 0.0204 g), and TEA (0.1194 mmol, 0.0121 g, 0.017 mL) was dissolved in 3 mL DCM for 5 minutes, then compound 13 in DCM was added. The mixture was stirred until full conversion of compound 13 was observed by TLC. LP220-p was purified by silica gel chromatography to yield 0.0400 g.

### Synthesis of LP221-p

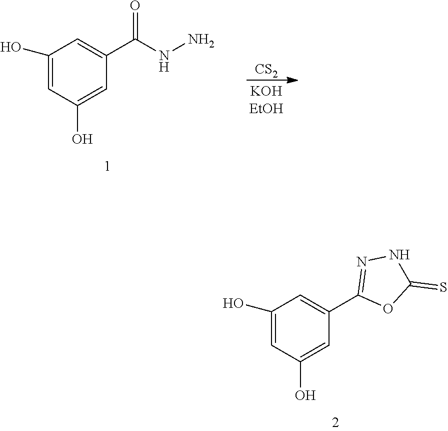

Carbon disulfide (75.0045 mmol, 5.7101 g, 4.532 mL) was slowly added to a solution of compound 1 (25 mmol, 4.20 g) and potassium hydroxide in EtOH (150 mL). The reaction mixture was refluxed for 24 hours. Upon completion, the solvent was evaporated under reduced pressure and the residue was dissolved in water. The aqueous solution was acidified to pH 2 using HCl. The product was extracted with EtOAc, and purified by silica gel chromatography using EtOAc/hexanes. After purification, 3.5 g of Compound 2 was obtained as an orange solid.

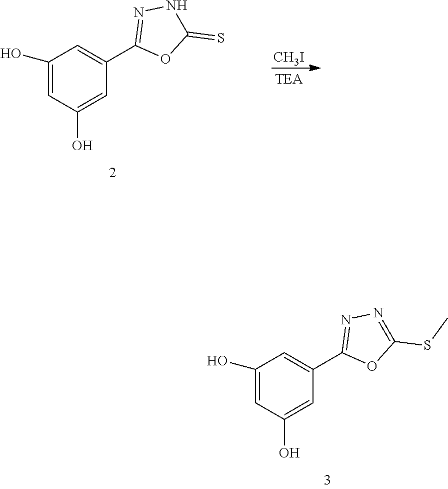

Compound 2 (10.0000 mmol, 2.1021 g) in THE (40 mL) was cooled to 0° C. CH₃I (11.0000 mmol, 1.5609 g, 0.685 mL) was added followed by TEA (10.1000 mmol, 1.0221 g, 1.408 mL). The reaction mixture was stirred for 4 hours. Upon completion, the solvent was quenched by NH₄C₁. The organic phase washed with brine, dried, and purified by silica gel chromatography to yield 1.5 g of compound 3.

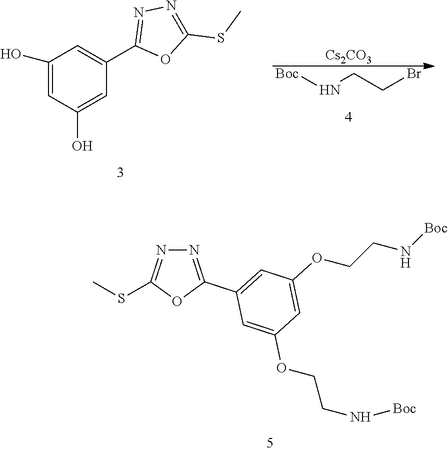

To a solution of compound 4 (6.8679 mmol, 1.5391 g) in 10 mL DMF was added compound 3 (3.1218 mmol, 0.7000 g) and Cs₂CO₃ (9.3654 mmol, 3.0514 g) at room temperature. The reaction mixture was stirred at 60° C. overnight. The reaction mixture was purified by standard work up (1N NaOH, Brine wash, and dried over Na₂SO₄) and silica gel chromatography to yield 1.0 g of compound 5.

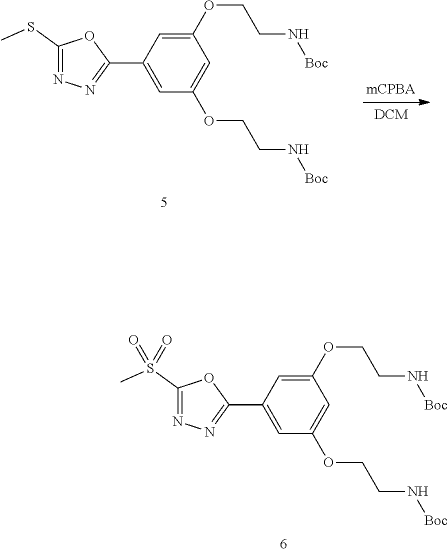 [SMILES: CSc1nnc(-c2cc(OCCNC(=O)OC(C)(C)C)cc(OCCNC(=O)OC(C)(C)C)c2)o1 ; CC(C)(C)OC(=O)NCCOc1cc(OCCNC(=O)OC(C)(C)C)cc(-c2nnc(S(C)(=O)=O)o2)c1]

A mixture of compound 5 (0.2000 mmol, 0.1021 g) and mCPBA (0.9998 mmol, 0.1725 g) in DCM was stirred until full conversion of mCPBA was observed by TLC. The reaction mixture was purified by standard work up (1N HCl, sat. NaHCO₃, Brine wash, and dried over Na₂SO₄) and silica gel chromatography to yield 0.05 g of compound 6.

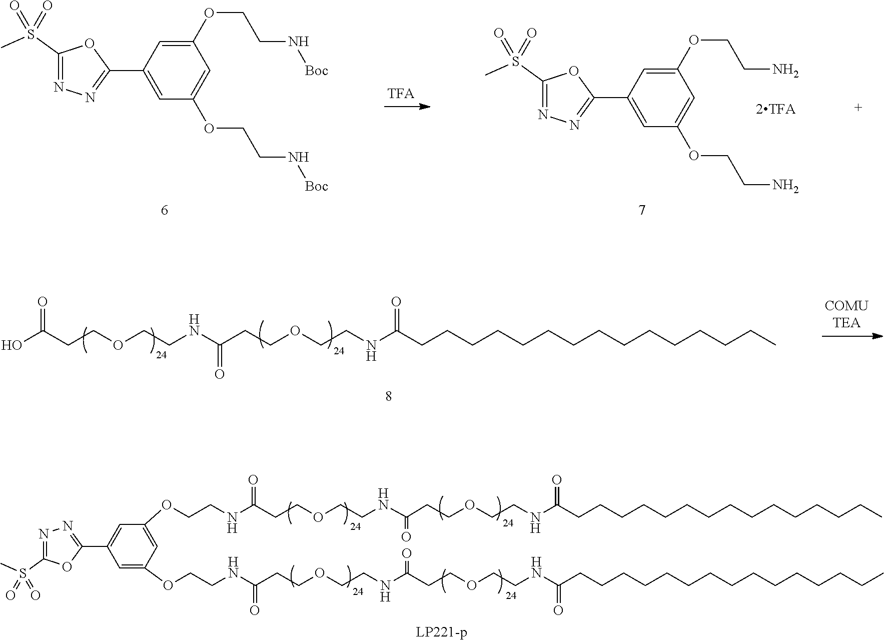

Compound 6 (0.0191 mmol, 0.0104 g) was dissolved in 1 mL DCM, and 0.5 mL of TFA was added and the mixture was stirred for 1 hour. All of the TFA was removed, and compound 7 was dried under vacuum for 1 hour. Compound 8 (0.0398 mmol, 0.1000 g), COMU (0.0477 mmol, 0.0204 g), and TEA (0.1990 mmol, 0.0201 g, 0.028 mL) was dissolved in 3 mL DCM for 5 minutes then compound 7 in DCM was added. The reaction mixture was stirred until full conversion of compound 7 was observed by TLC. The residue was purified by silica gel chromatography to yield 0.016 g of LP221-p.

### Synthesis of LP223-p

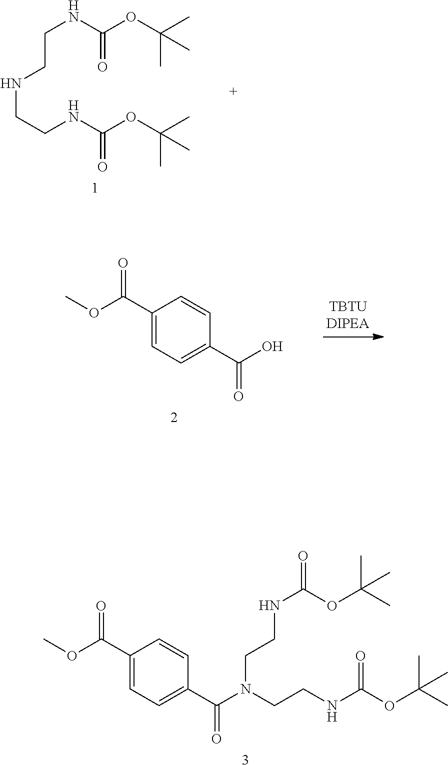

To a solution of compound 1 (741 mg, 2.442 mmol, 1.0 equiv.), compound 2 (528 mg, 2.930 mmol, 1.20 equiv.) and diisopropylethylamine (1.276 mL, 7.327 mmol, 3.0 equiv.) in anhydrous DMF (10 mL) was added TBTU (980 mg, 3.052 mmol, 1.25 equiv.) at room temperature. The reaction mixture was kept at room temperature for 2 hours. The organic phase was quenched with saturated sodium bicarbonate aqueous solution (10 mL) and extracted with EtOAc (2×10 mL). The organic phases were combined, dried over anhydrous Na₂SO₄, and concentrated. Compound 3 was purified by CombiFlash and was eluted with 40-80% EtOAc in hexanes. LC-MS: [M+H]+, calculated 466.25, found 466.72.

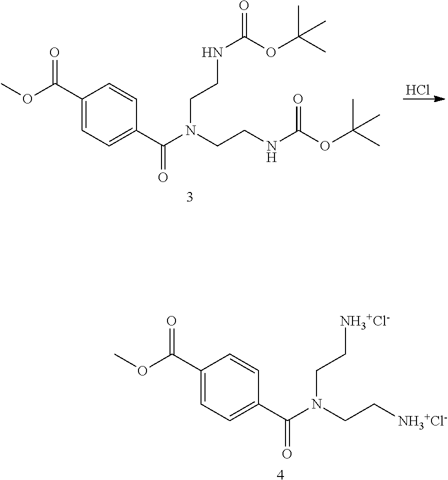

To compound 3 (990 mg, 2.126 mmol, 1.0 equiv.) was added 4M HCl in dioxane (6.38 mL, 25.518 mmol, 12 equiv.) at room temperature. The reaction mixture was kept at room temperature for 1 hour and then concentrated. Compound 4 was used directly without further purification. LC-MS: [M+H]/+ calculated 266.14, found 266.43.

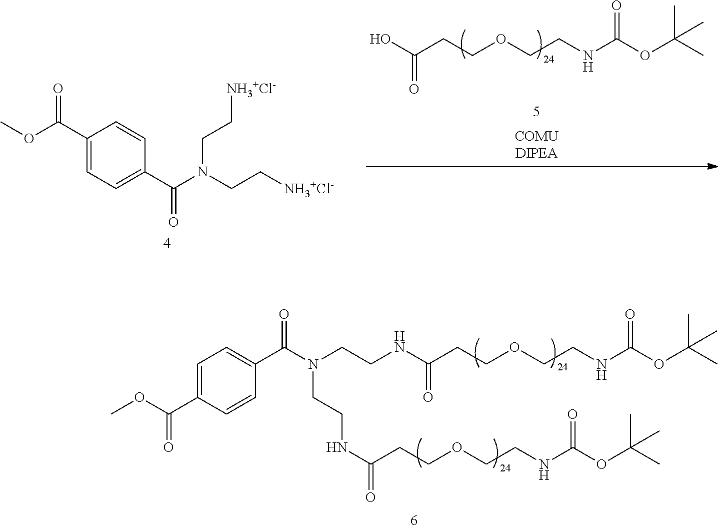

To a solution of compound 4 (100 mg, 0.295 mmol, 1.0 equiv.), compound 5 (755 mg, 0.606 mmol, 2.05 equiv.) and diisopropylethylamine (0.257 mL, 0.025 mmol, 5.0 equiv.) in anhydrous DCM (10 mL) was added COMU (278 mg, 0.650 mmol, 2.20 equiv.) at room temperature. The reaction mixture was kept at room temperature for 1 hour. The reaction mixture was washed with saturated ammonium chloride (10 mL) and sodium bicarbonate aqueous solution (10 mL). The organic phase was dried over anhydrous Na₂SO₄ and concentrated. Compound 6 was purified by CombiFlash eluting with 8-18% MeOH in DCM. LC-MS: [M+3H]/3, calculated 907.86, found 907.61.

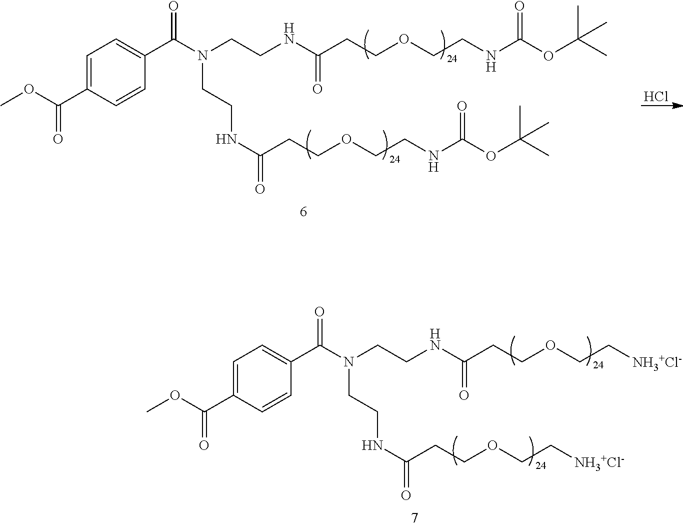

To compound 6 (550 mg, 0.202 mmol, 1.0 equiv.) was added 4M HCl in dioxane (1.01 mL, 4.040 mmol, 20 equiv.) at room temperature. The reaction mixture was kept at room temperature for 1 hour and then concentrated. Compound 7 was used directly without further purification. LC-MS: [M+H]/+ calculated 841.16, found 842.20.

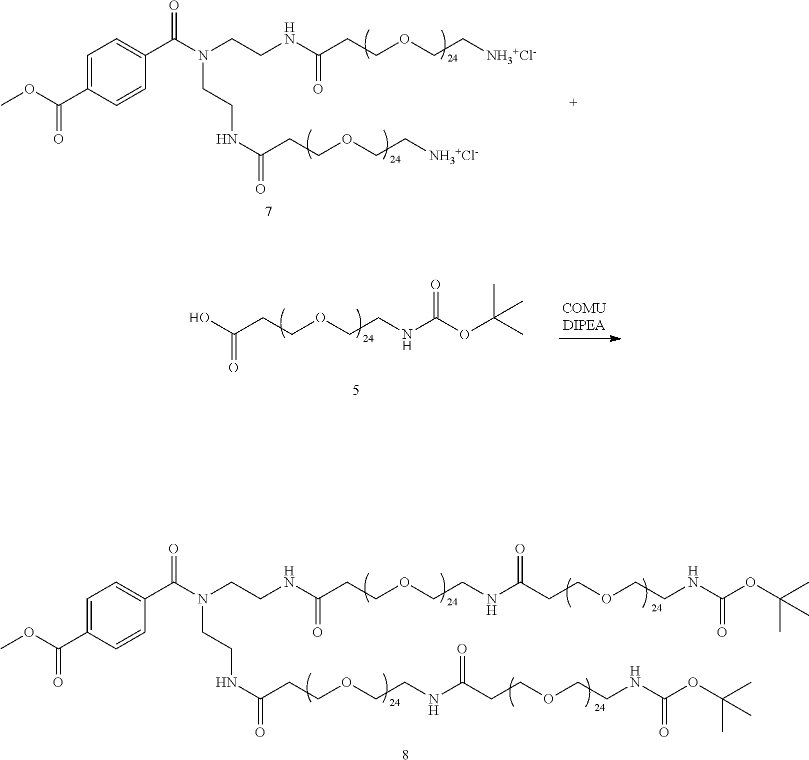

To a solution of compound 1 (490 mg, 0.188 mmol, 1.0 equiv.), compound 5 (482 mg, 0.387 mmol, 2.05 equiv.) and diisopropylethylamine (0.164 mL, 0.944 mmol, 5.0 equiv.) in anhydrous DCM (10 mL) was added COMU (177 mg, 0.415 mmol, 2.20 equiv.) at room temperature. The reaction mixture was kept at room temperature for 1 hour. The reaction mixture was washed with saturated ammonium chloride (10 mL) and sodium bicarbonate aqueous solution (10 mL). The organic phase was dried over anhydrous Na₂SO₄ and concentrated. Compound 8 was purified by CombiFlash eluting with 8-20% MeOH in DCM. LC-MS: [M+5H]/5, calculated 960.18, found 961.74.

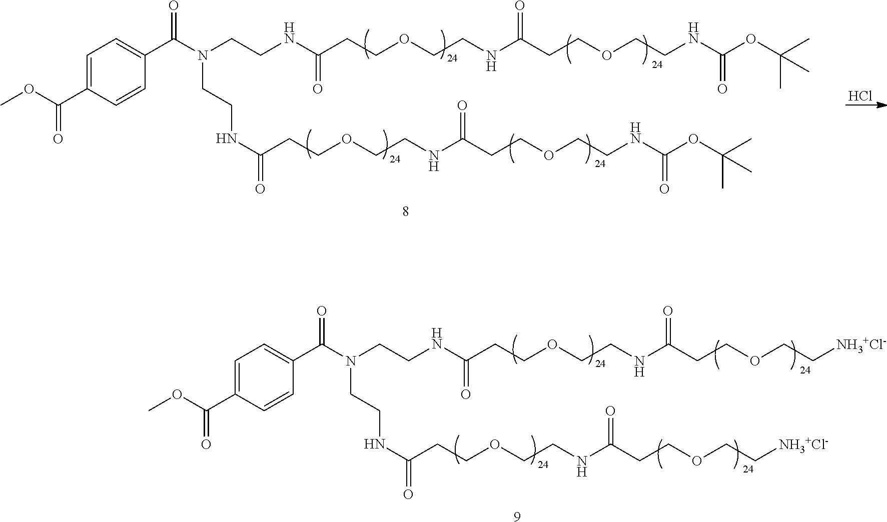

To compound 1 (670 mg, 0.134 mmol, 1.0 equiv.) was added 4M HCl in dioxane (0.673 mL, 2.691 mmol, 20 equiv.) at room temperature. The reaction mixture was kept at room temperature for 1 hour and then concentrated. Compound 9 was used directly without further purification. LC-MS: [M+5H]/5 calculated 956.16, found 957.66.

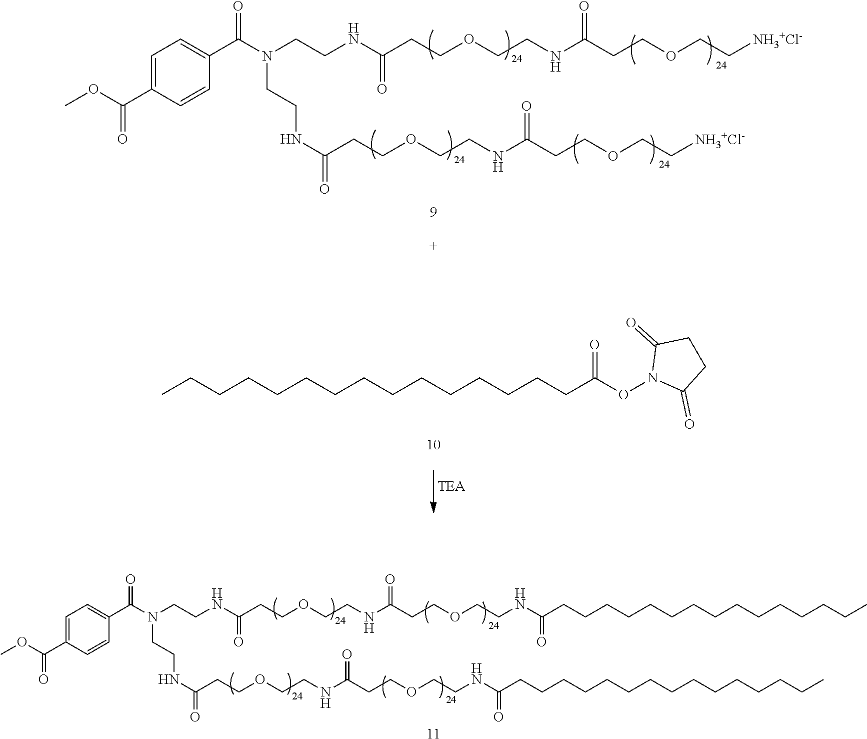

To a solution of compound 9 (650 mg, 0.134 mmol, 1.0 equiv.) and compound 10 (106 mg, 0.301 mmol, 2.25 equiv.) in anhydrous DCM (20 mL) was added TEA (0.095 mL, 0.669 mmol, 5.0 equiv.) at room temperature. The reaction mixture was kept at room temperature for 2 hours and the solvent was concentrated. Compound 11 was separated by CombiFlash eluting with 8-20% MeOH in DCM. LC-MS: calculated [M+5H]/5 1051.45, found 1053.44.

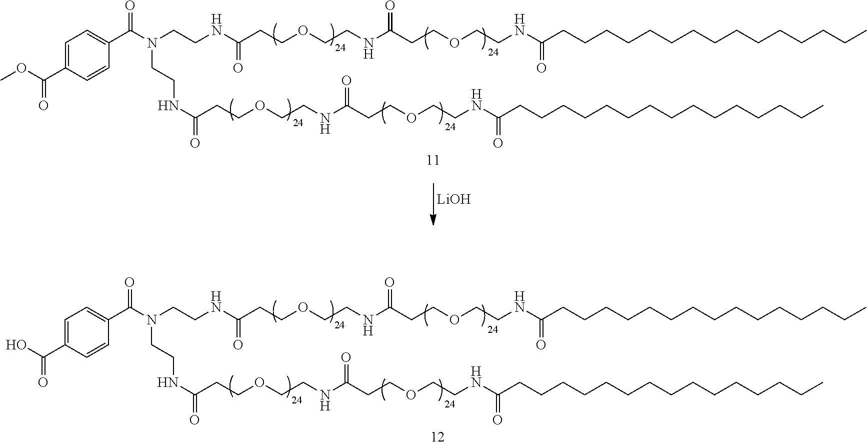

To a solution of compound 11 (460 mg, 0.0875 mmol, 1.0 equiv.) in THE (5 mL) and water (5 mL) was added LiOH (10.5 mg, 0.437 mmol, 5.0 equiv.) at room temperature. The reaction mixture was kept at room temperature for 1 hour. The reaction mixture pH was adjusted to 3.0 by adding HCl and was extracted with DCM (2×10 mL). The combined organic phases were dried over anhydrous Na₂SO₄ and concentrated. Compound 12 was used directly without further purification. LC-MS: calculated [M+5H]+/5 1048.65, found 1050.68.

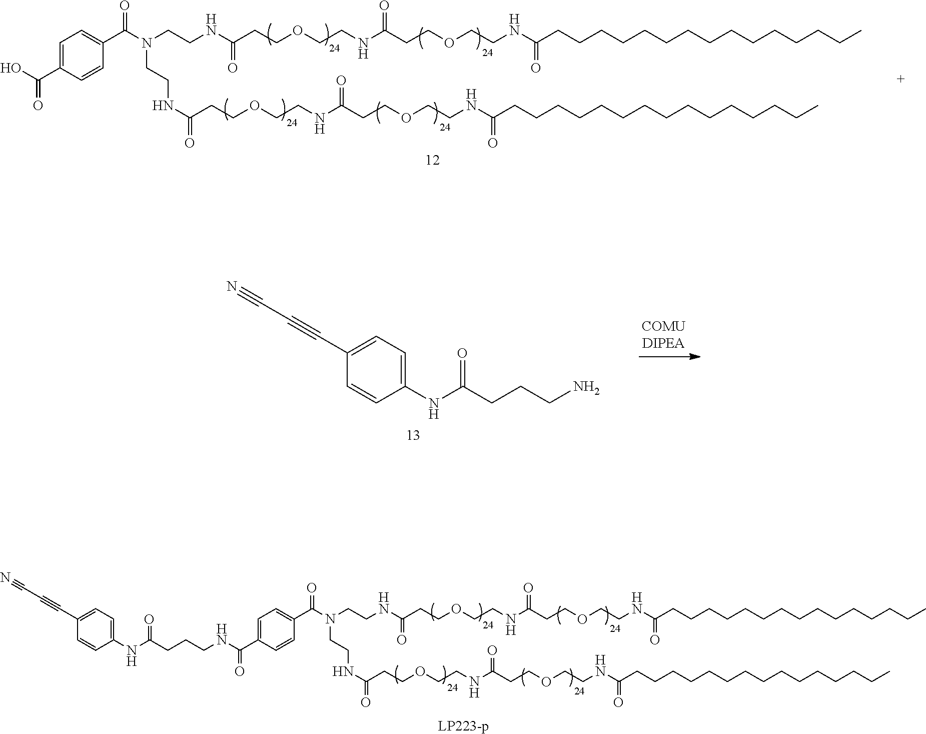

To a solution of compound 12 (100 mg, 0.0191 mmol, 1.0 equiv.), compound 13 (4.8 mg, 0.021 mmol, 1.1 equiv.) and diisopropylethylamine (0.010 mL, 0.0572 mmol, 3.0 equiv.) in anhydrous DCM (3 mL) was added COMU (10.2 mg, 0.0238 mmol, 1.25 equiv.) at room temperature. The reaction mixture was kept at room temperature for 1 hour. The reaction mixture was washed with saturated sodium bicarbonate aqueous solution (5 mL). The organic phase was dried over anhydrous Na₂SO₄ and concentrated. LP223-p was purified by CombiFlash eluting with 8-20% MeOH in DCM. LC-MS: [M+5H]/5, calculated 1090.47, found 1091.85.

### Synthesis of LP224-p

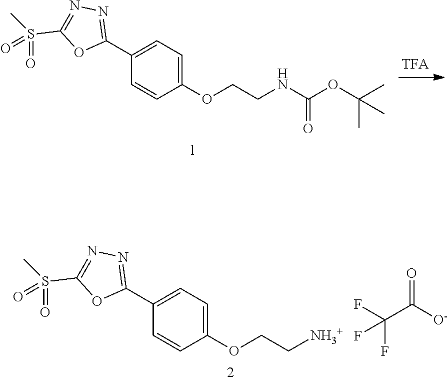

To solution of compound 1 (12 mg, 0.0313 mmol, 1.0 equiv.) in DCM (1 mL) was added TFA (0.5 mL) at room temperature. The reaction mixture was kept at room temperature for 30 minutes and then concentrated. Compound 2 was used directly without further purification. LC-MS: [M+H]+ calculated 284.06, found 284.26.

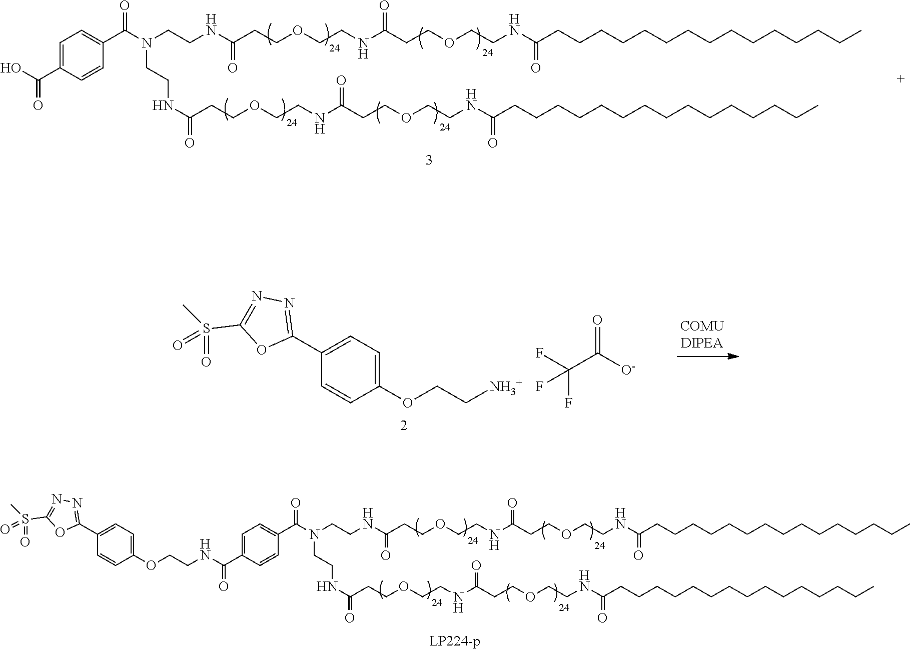

To a solution of compound 3 (150 mg, 0.0286 mmol, 1.0 equiv., compound 12 from LP223-p synthesis), compound 2 (12.5 mg, 0.0315 mmol, 1.1 equiv.) and diisopropylethylamine (0.015 mL, 0.0859 mmol, 3.0 equiv.) in anhydrous DCM (3 mL) was added COMU (15.3 mg, 0.0358 mmol, 1.25 equiv.) at room temperature. The reaction mixture was kept at room temperature for 1 hour. The reaction mixture was washed with saturated sodium bicarbonate aqueous solution (5 mL). The organic phase was dried over anhydrous Na₂SO₄ and concentrated. LP224-p was purified by CombiFlash eluting with 8-16% MeOH in DCM. LC-MS: [M+5H]/5, calculated 1101.66, found 1103.13.

### Synthesis of LP225-p

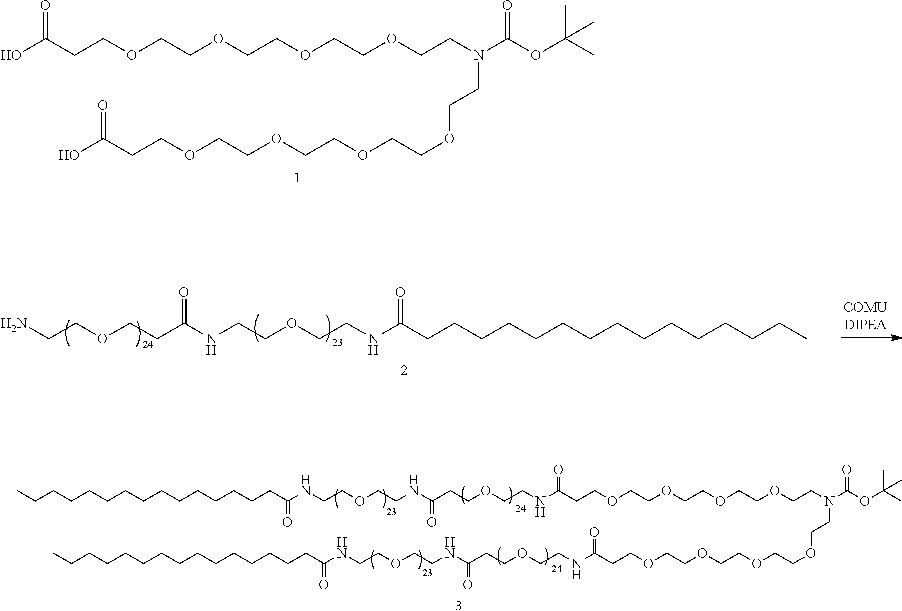

To a solution of compound 1 (80 mg, 0.130 mmol, 1.0 equiv.), compound 2 (652 mg, 0.267 mmol, 2.05 equiv.), and diisopropylethylamine (0.068 mL, 0.391 mmol, 3.0 equiv.) in anhydrous DCM (10 mL) was added COMU (134 mg, 0.312 mmol, 2.40 equiv.) at room temperature. The reaction mixture was kept at room temperature overnight. Compound 3 was purified by CombiFlash eluting with 8-16% MeOH in DCM. LC-MS: [M+5H]/5, calculated 1091.89, found 1093.41.

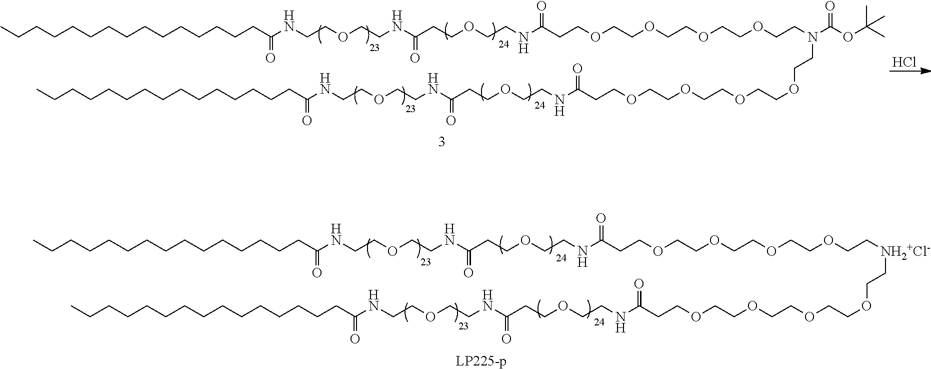

To compound 3 (340 mg, 0.0623 mmol, 1.0 equiv.) was added 4M HCl in dioxane (0.311 mL, 1.245 mmol, 20 equiv.) at room temperature. The reaction mixture was kept at room temperature for 1 hour and then concentrated. Compound 4 was used directly without further purification. LC-MS: [M+5H]/5 calculated 1071.88, found 1073.36.

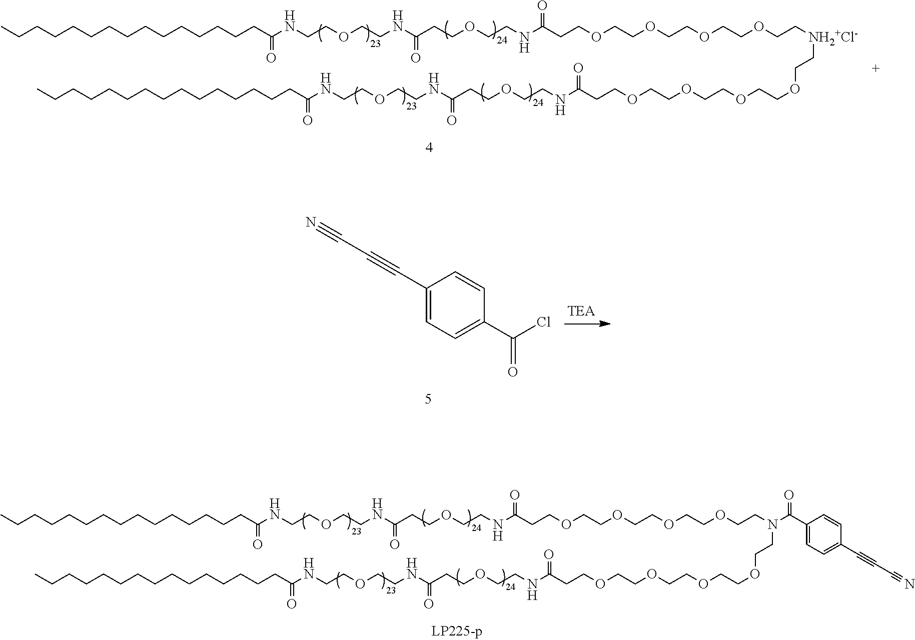

To a solution of compound 4 (100 mg, 0.0185 mmol, 1.0 equiv.) and compound 5 (3.9 mg, 0.0204 mmol, 1.10 equiv.) in anhydrous DCM (2 mL) was added TEA (0.008 mL, 0.0556 mmol, 3.0 equiv.) at room temperature. The reaction mixture was kept at room temperature for 2 hours and the solvent was concentrated. LP225-p was separated by CombiFlash eluting with 13-20% MeOH in DCM. LC-MS: calculated [M+5H]/5 1102.48, found 1104.45.

### Synthesis of LP226-p

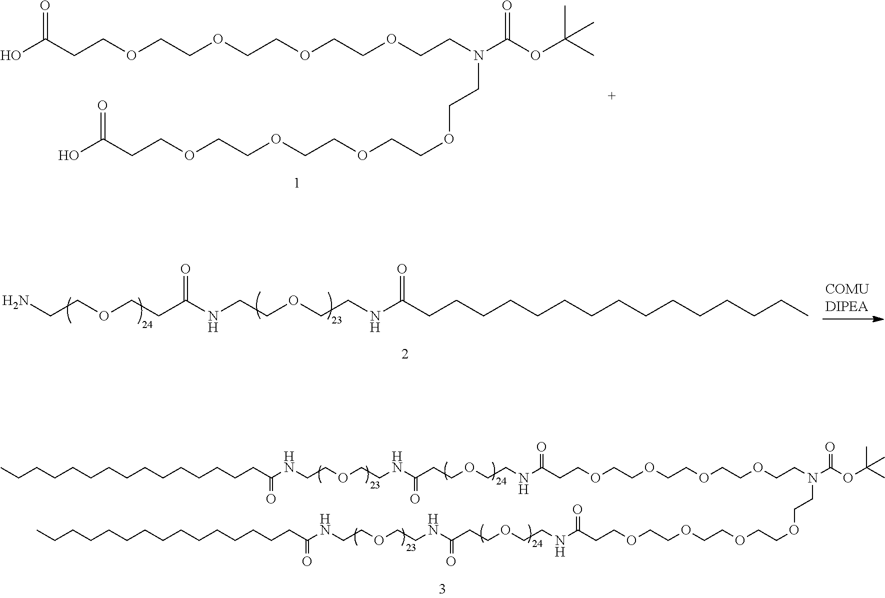

To a solution of compound 1 (80 mg, 0.130 mmol, 1.0 equiv.), compound 2 (652 mg, 0.267 mmol, 2.05 equiv.) and diisopropylethylamine (0.068 mL, 0.391 mmol, 3.0 equiv.) in anhydrous DCM (10 mL) was added COMU (134 mg, 0.312 mmol, 2.40 equiv.) at room temperature. The reaction mixture was kept at room temperature overnight. Compound 3 was purified by CombiFlash eluting with 8-16% MeOH in DCM. LC-MS: [M+5H]/5, calculated 1091.89, found 1093.41.

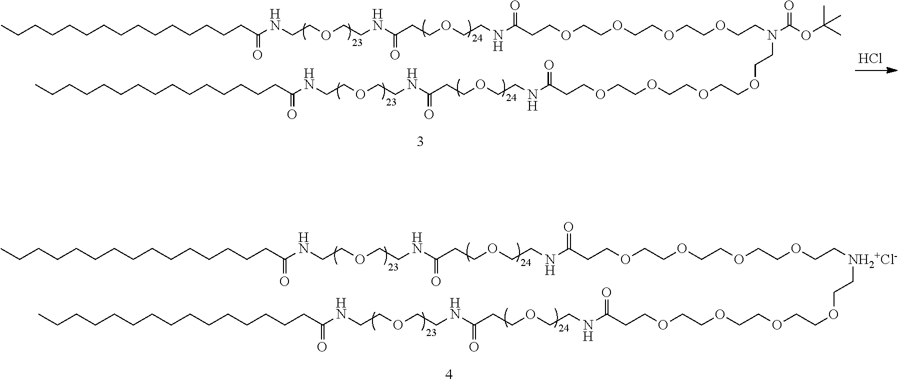

To compound 3 (340 mg, 0.0623 mmol, 1.0 equiv.) was added 4M HCl in dioxane (0.311 mL, 1.245 mmol, 20 equiv.) at room temperature. The reaction mixture was kept at room temperature for 1 hour and then concentrated. Compound 4 was used directly without further purification. LC-MS: [M+5H]/5 calculated 1071.88, found 1073.36.

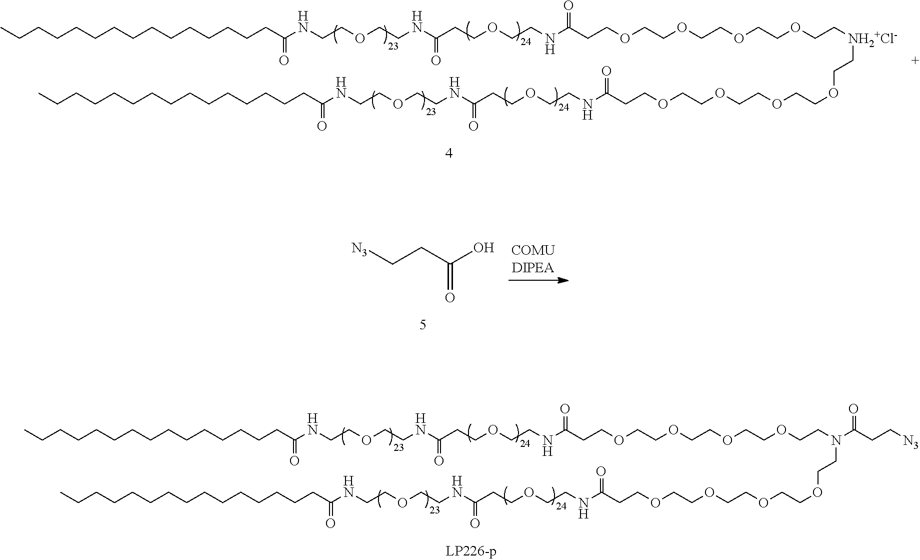

To a solution of compound 4 (80 mg, 0.0148 mmol, 1.0 equiv.), compound 5 (1.9 mg, 0.0163 mmol, 1.1 equiv.), and diisopropylethylamine (0.008 mL, 0.0445 mmol, 3.0 equiv.) in anhydrous DCM (2 mL) was added COMU (7.9 mg, 0.0185 mmol, 1.25 equiv.) at room temperature. The reaction mixture was kept at room temperature for 1 hour. The reaction mixture was washed with saturated sodium bicarbonate aqueous solution (5 mL). The organic phase was dried over anhydrous Na₂SO₄ and concentrated. LP226-p was purified by CombiFlash eluting with 15-20% MeOH in DCM. LC-MS: [M+5H]/5, calculated 1091.28, found 1093.41.

### Synthesis of LP238-p

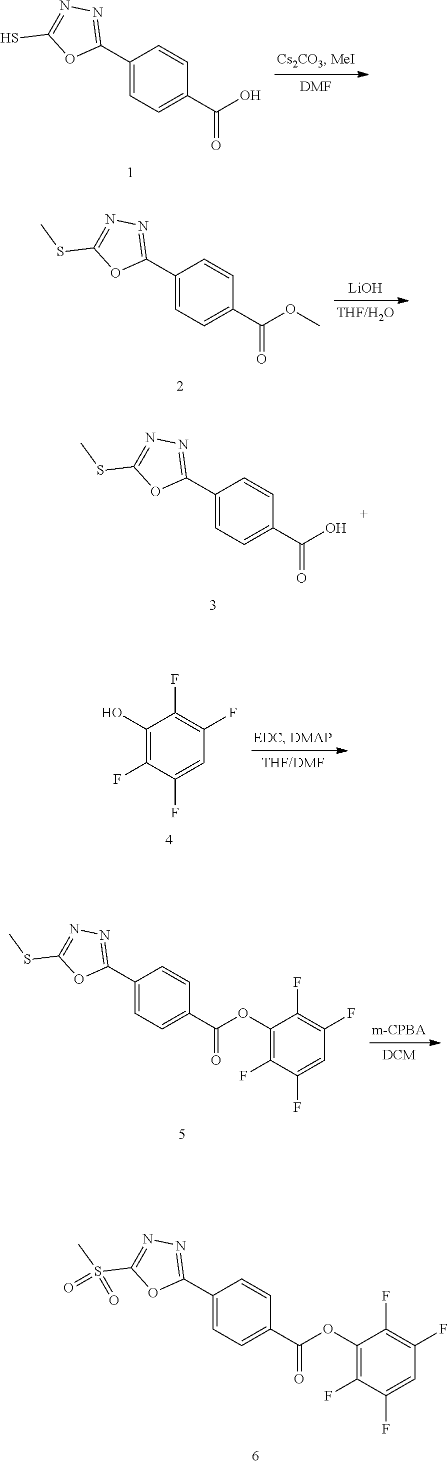

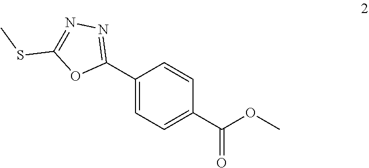

To a suspension of compound 1 (5.00 g, 22.50 mmol) and Cs₂CO₃ (25.66 g, 78.75 mmol) in anhydrous DMF (80 mL) was added methyl iodide (4.20 mL, 67.50 mmol) at room temperature. The reaction mixture was stirred at room temperature for 48 hours. The reaction was quenched with water (200 mL) and the mixture was extracted with EtOAc (3×100 mL). The organic phase was combined and washed with water and brine. The organic layer was dried over anhydrous Na₂SO₄ and concentrated. Compound 2 was obtained as a light yellow solid, 5.41 g, 96%. Compound 2 was used directly without further purification. LC-MS: [M+H] calculated 251.05, found 251.18.

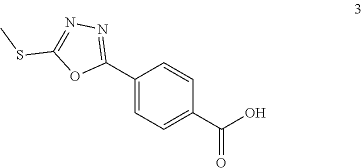 [SMILES: CSc1nnc(-c2ccc(C(=O)O)cc2)o1]

To a solution of compound 2 (5.41 g, 21.62 mmol) in THF/H₂O (50 mL/50 mL) was added LiOH (2.59 g, 108.08 mmol) at room temperature. The reaction mixture was stirred at room temperature for 1 hour. After removing THE under vacuum, the pH was adjusted to −2 by [C] HCl. Then EtOAc (3×60 mL) was used to extract. The organic layers were combined, washed with brine, then dried over anhydrous Na₂SO₄, and concentrated. Compound 3 was obtained as an off-white solid, 5 g, 98%. Compound 3 was used directly without further purification. LC-MS: calculated [M+H] 237.03, found 237.26.

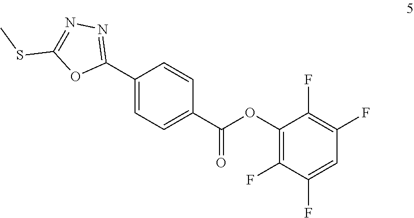 [SMILES: CSc1nnc(-c2ccc(C(=O)Oc3c(F)c(F)cc(F)c3F)cc2)o1]

To a solution of compound 3 (5.81 g, 24.60 mmol) in THF/DMF (80 mL/20 mL) was added EDC (7.07 g, 36.90 mmol), DMAP (0.30 g, 2.46 mmol) and compound 4 (6.13 g, 36.90 mmol) at room temperature. The reaction mixture was stirred at room temperature overnight. After removing solvent under vacuum, the residue was loaded on a 120 g column and compound 5 was eluted with 0-50% EtOAc in hexanes. Compound 5 was obtained as a white solid, 9.36 g, 99%. LC-MS: calculated [M+H] 385.03, found 385.46.

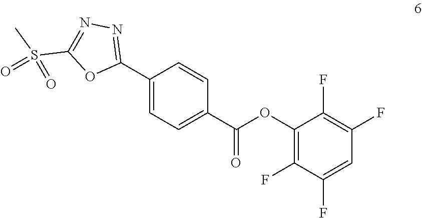 [SMILES: CS(=O)(=O)c1nnc(-c2ccc(C(=O)Oc3c(F)c(F)cc(F)c3F)cc2)o1]

To a solution of compound 5 (2.29 g, 5.96 mmol) in DCM (110 mL) was added 70% m-CPBA (5.14 g, 27.79 mmol) at 0° C. The reaction mixture was stirred at room temperature for 6 hours. Another 1.8 g m-CPBA was added at room temperature. The reaction mixture was stirred at room temperature overnight. After filtration, the solvent was removed under vacuum. The residue was recrystallized from DCM/EtOAc (50 mL/50 mL) twice. Compound 6 was obtained as white needle crystal, 1.93 g, 78%. LC-MS: calculated [M+H] 417, found 417.

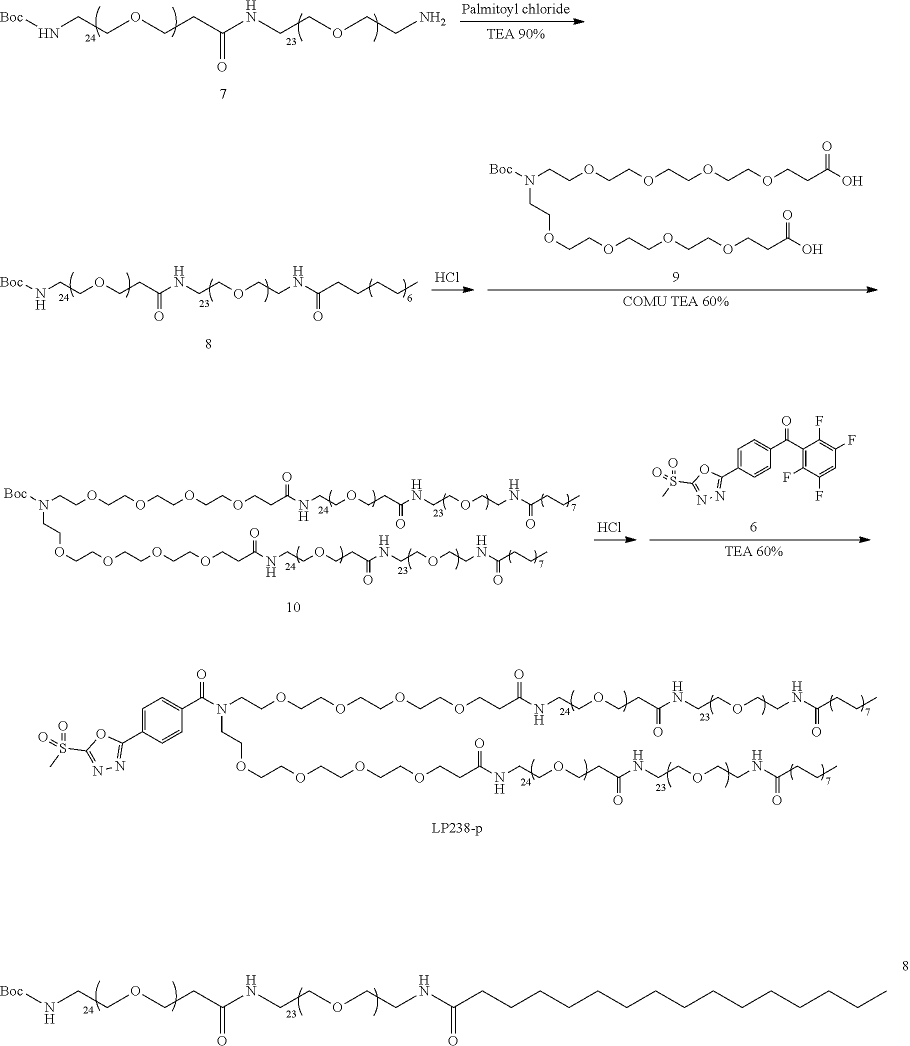

To a solution of compound 7 (10.00 g, 4.34 mmol) in DCM (100 mL) was added palmitoyl chloride (1.31 g, 4.78 mmol) and TEA at 0° C. The reaction mixture was stirred at room temperature overnight and then the solvent was removed under vacuum. The residue was purified by silica gel chromatography using 0-20% MeOH in DCM. Compound 8 was obtained as a white solid, 10.0 g, 90%.

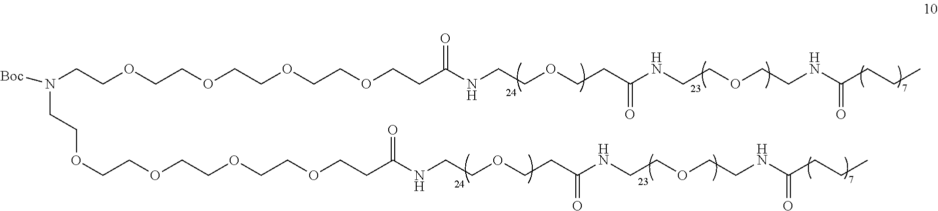

Compound 8 (9.56 g, 3.76 mmol) was dissolved in 25 mL 4N HCl/dioxane and stirred at room temperature for 1 hour. All solvent was removed and the residue was dried under vacuum for 2 hours. The residue was re-dissolved in 150 mL DCM and TEA was added, followed by compound 9 (1.10 g, 1.79 mmol), and COMU (1.69 g, 3.94 mmol). The reaction mixture was stirred at room temperature overnight. After a standard workup (1N HCl, Sat. bicarb, brine wash), DCM was removed. Compound 10 was purified by a 120 g column using 0-20% MeOH in DCM to obtain 5.90 g, 60%.

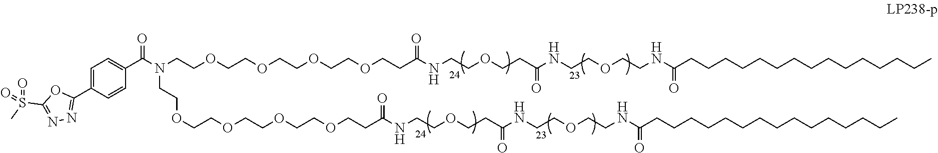

Compound 10 (4.50 g, 0.82 mmol) was dissolved in 20 mL 4N HCl/dioxane and stirred at room temperature for 1 hour. All solvent was removed and the residue was dried under vacuum for 2 hours. The residue was re-dissolved in 100 mL DCM and TEA was added, followed by compound 6 (0.69 g, 1.65 mmol). The reaction mixture was stirred at room temperature overnight. TEA was removed by a 1H HCl wash and the organic layer was concentrated. Crude LP238-p was purified by silica gel chromatography using 0-20% MeOH in DCM. 2.80 g (60%) of LP238-p was obtained as a light yellow solid.

### Synthesis of LP240-p

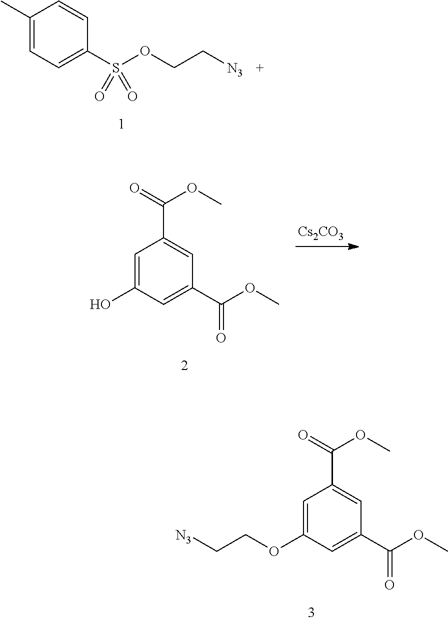

To a suspension of compound 1 (880 mg, 3.647 mmol, 1.0 equiv.) and Cs₂CO₃ (1.782 g, 5.471 mmol, 1.50 equiv.) in anhydrous DMF (10 mL) was added compound 2 (0.843 g, 4.012 mmol, 1.10 equiv.) at room temperature. The reaction mixture was kept at room temperature for 3 hours. The reaction was quenched with water (20 mL) and the mixture was extracted with ethyl acetate (2×10 mL). The combined organic phases were washed with brine (1×20 mL) and water (1×20 mL). The organic phase was dried through anhydrous Na₂SO₄ and concentrated. Compound 3 was used directly without further purification. LC-MS: [M+H]+ calculated 280.09, found 280.39.

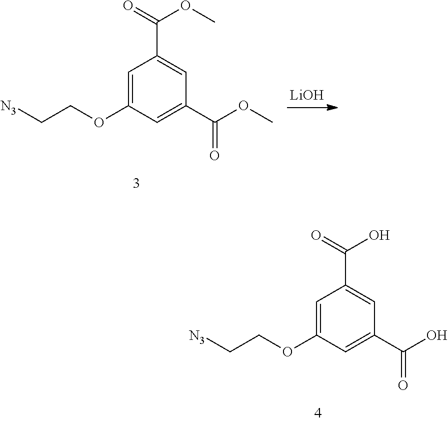

To a solution of compound 3 (1000 mg, 3.581 mmol, 1.0 equiv.) in THE (10 mL) and water (10 mL) was added LiOH (686 mg, 19.978 mmol, 8.0 equiv.) at room temperature. The reaction mixture was kept at room temperature for 1 hour. The reaction mixture pH was adjusted to 1.0 by adding HCl. The product was extracted with ethyl acetate (2×10 mL). The combined organic phases were dried over anhydrous Na₂SO₄ and concentrated. Compound 4 was used directly without further purification. LC-MS: calculated [M+H]+ 252.05, found 251.31.

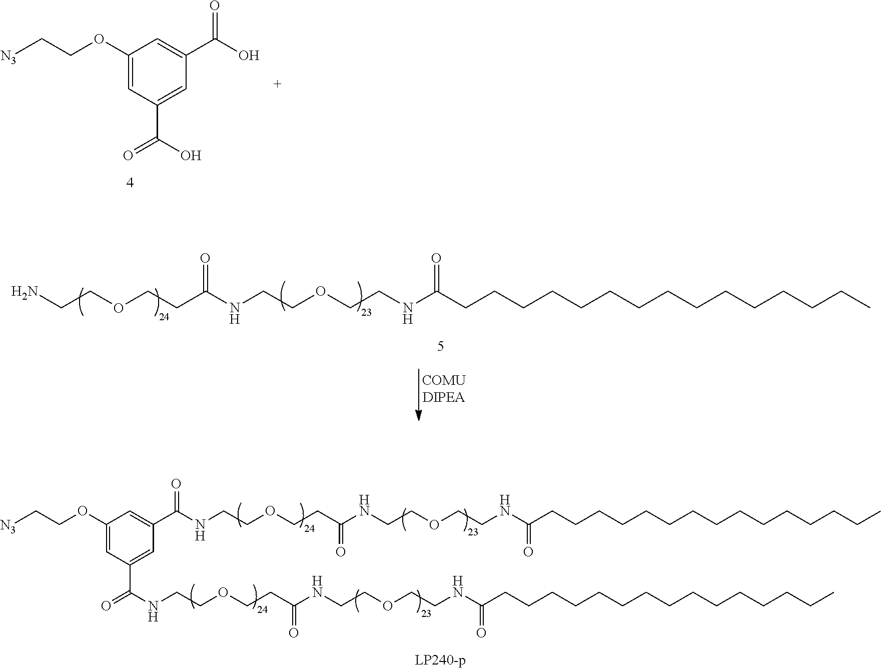

To a solution of compound 4 (5 mg, 0.0199 mmol, 1.0 equiv.), compound 5 (100 mg, 0.0408 mmol, 2.05 equiv.), and diisopropylethylamine (0.017 mL, 0.0995 mmol, 5.0 equiv.) in anhydrous DCM (2 mL) was added COMU (20.5 mg, 0.0478 mmol, 2.40 equiv.) at room temperature. The reaction mixture was kept at room temperature overnight. LP240-p was purified by CombiFlash eluting with 8-20% MeOH in DCM. LC-MS: [M+5H]/5, calculated 1019.43, found 1020.79.

### Synthesis of LP246-p

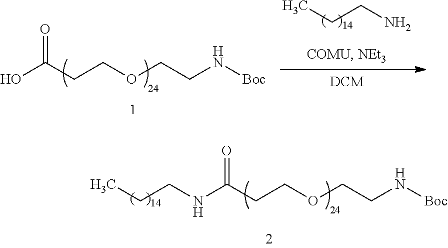

Compound 1 (0.5 g, 0.401 mmol) and COMU (0.206 g, 0.481 mmol) were dissolved in DCM (10 mL) and NEt₃ (0.168 mL, 1.2 mmol) was added. The resulting solution was stirred for 10 minutes. After 10 minutes 1-aminohexadecane (0.102 g, 0.42 mmol) was added to the solution of compound 1 and COMU. The resulting solution was stirred for 90 minutes and then checked by LC-MS. The reaction was quenched with 5 mL of water and stirred for 5 minutes. The layers were separated and the organic layer was washed with 1 M HCl_{(aq)} (2×15 mL), sat. NaHCO_{3(aq)} (2×20 mL), water (20 mL), sat. NaCl(aq) (2×20 mL), dried over Na₂SO₄ and concentrated to yield a foamy light yellow solid. Crude product was purified by silica gel chromatography 0-20% MeOH in DCM. Pure fractions of compound 2 were combined to yield 515 mg (87% yield) as a white solid.

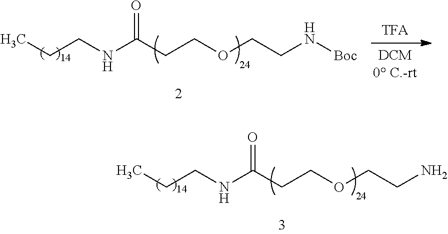

Compound 2 (0.515 g, 0.35 mmol) was dissolved in DCM (4 mL), cooled to 0° C., and TFA (1 mL, 13 mmol) was added. After the addition of the TFA, the reaction was allowed to warm to room temperature. The resulting solution was stirred for 90 minutes and then analyzed by LC-MS. The reaction was quenched with the addition of sat. NaHCO_{3(aq)}until no effervescence was observed and stirred for 5 minutes. The layers were separated, and the organic layer was washed with sat. NaHCO₃(aq) (2×20 mL), water (20 mL), sat. NaCl(aq) (20 mL), dried over Na₂SO₄ and concentrated to yield compound 3 as a foamy white solid 0.4674 g (97.4% yield).

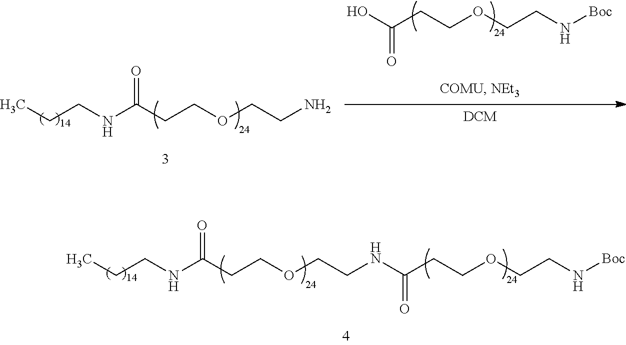

^{t}Boc-amido-PEG₂₄-COOH (0.524 g, 0.42 mmol) and COMU (0.180 g, 0.42 mmol) were dissolved in DCM (10 mL) and NEt₃ (0.488 mL, 3.5 mmol) was added. The resulting solution was stirred for 10 minutes. After 10 minutes, compound 3 (0.480 g, 0.35 mmol) was added to the solution of tBoc-amido-PEG₂₄-COOH. The resulting solution was stirred for 1 hour and checked with LC-MS. The reaction was quenched with 5 mL of water and stirred for 5 minutes. The layers were separated, and the organic layer was washed with 1 M HCl (1×15 mL), sat. NaHCO₃(aq) (2×20 mL), Water (20 mL), 1 M HCl (1×20 mL), sat. NaCl_{(aq)} (2×20 mL), dried over Na₂SO₄ and concentrated to yield a foamy light yellow solid (ca. 900 mg). Crude product was purified by silica gel chromatography 0-20% MeOH in DCM. Compound 4 eluted at 4% MeOH in DCM. Pure fractions of compound 4 were combined to yield 0.780 g (85.7%) as a light pink solid.

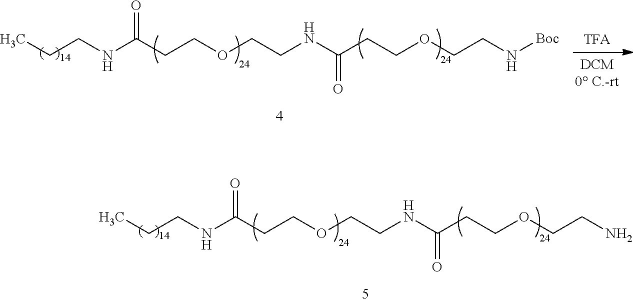

Compound 4 (0.78 g, 0.3 mmol) was dissolved in DCM (4 mL), cooled to 0° C., and TFA (1 mL, 13 mmol) was added. After the addition of the TFA, the reaction was allowed to warm to room temperature. The resulting solution was stirred for 3 hours and checked by LC-MS. The reaction was quenched with the addition sat. NaHCO_{3(aq)}until no effervescence was observed and stirred for 5 minutes. The layers were separated and the organic layer was washed with sat. NaHCO_{3(aq)} (2×20 mL), water (20 mL), sat. NaCl_{(aq)} (20 mL), dried over Na₂SO₄ and concentrated to yield compound 5 as a foamy white solid 0.741 g (98.9% yield). Compound 5 was used in the next step without further purification.

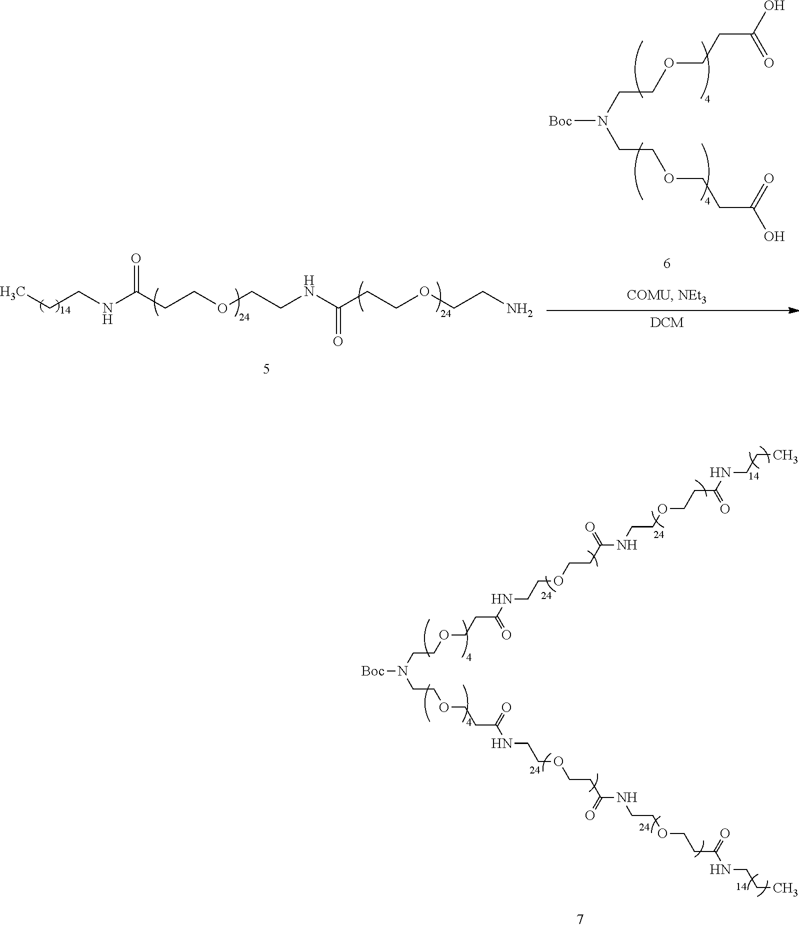

N-Boc-N-Bis-PEG₄-Acid (compound 6, 0.0339 g, 0.055 mmol) and COMU (0.0473 g, 0.11 mmol) were dissolved in DCM (3 mL) and NEt₃ (0.167 mL, 1.20 mmol) was added. The resulting solution was stirred for 10 minutes. After 10 minutes compound 5 (0.30 g, 0.12 mmol) was added to the solution of compound 6. The resulting solution was stirred for 1 hour. The reaction mixture was concentrated and loaded directly onto a silica gel column for purification. Crude product was purified by silica gel chromatography 0-20% MeOH in DCM. Compound 7 began eluting with 6% MeOH in DCM. The majority of pure compound 7 eluted with 12% McOH in DCM. Pure fractions of compound 7 were combined to yield 264 mg (86% yield) as an off-white solid.

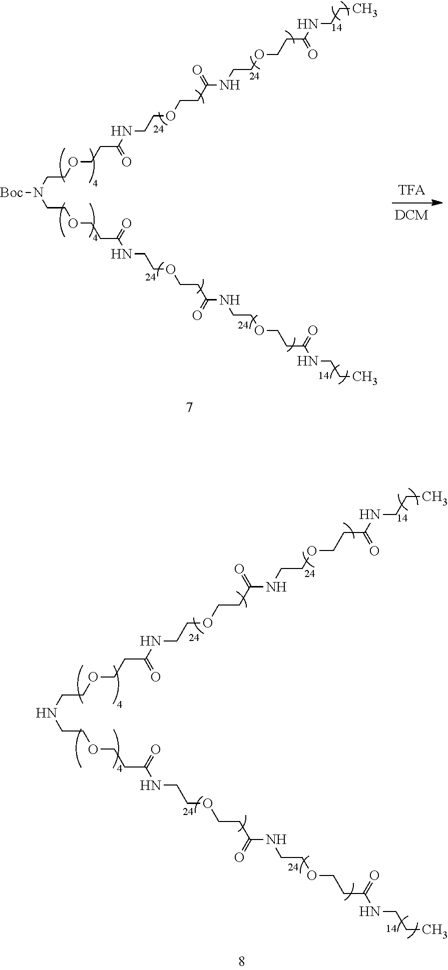

Compound 7 (100 mg, 0.041 mmol) was dissolved in DCM (2 mL) and TFA (1 mL, 8.64 mmol) was added. The reaction mixture was stirred for 2 hours and checked by LC-MS. The reaction was quenched with sat. NaHCO₃(aq) and diluted with DCM. The layers were separated and the organic layer was washed with sat. NaCl(aq) (20 mL), dried over Na₂SO₄ and concentrated to yield 0.09 g of compound 8 as a light yellow solid (86% yield). Compound 8 was used directly in the next step without further purification.

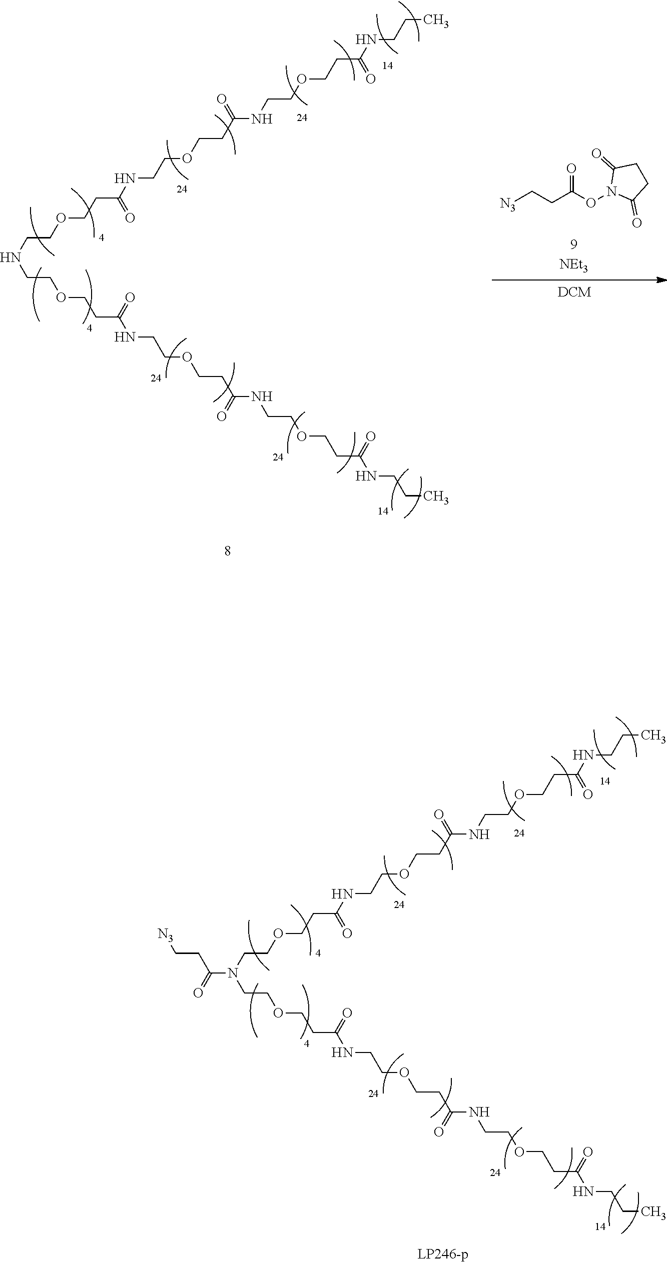

Compound 8 (0.090 g, 0.016 mmol) was dissolved in DCM (3 mL) and NEt₃ (22.9 μL, 0.164 mmol) was added followed by the addition of 3-azido propiponate NHS-ester (compound 9, 0.0174 g, 0.082 mmol). The reaction mixture was stirred for 4 hours and checked by LC-MS. The reaction was concentrated and loaded directly onto a silica gel column for purification. Crude product was purified by silica gel chromatography (4 g gold redisep column) 0-20% MeOH in DCM. LP246-p eluted with 16% MeOH in DCM. Pure fractions of LP246-p were combined to yield 0.019 g of an off-white solid (20.7% yield).

### Synthesis of LP247-p

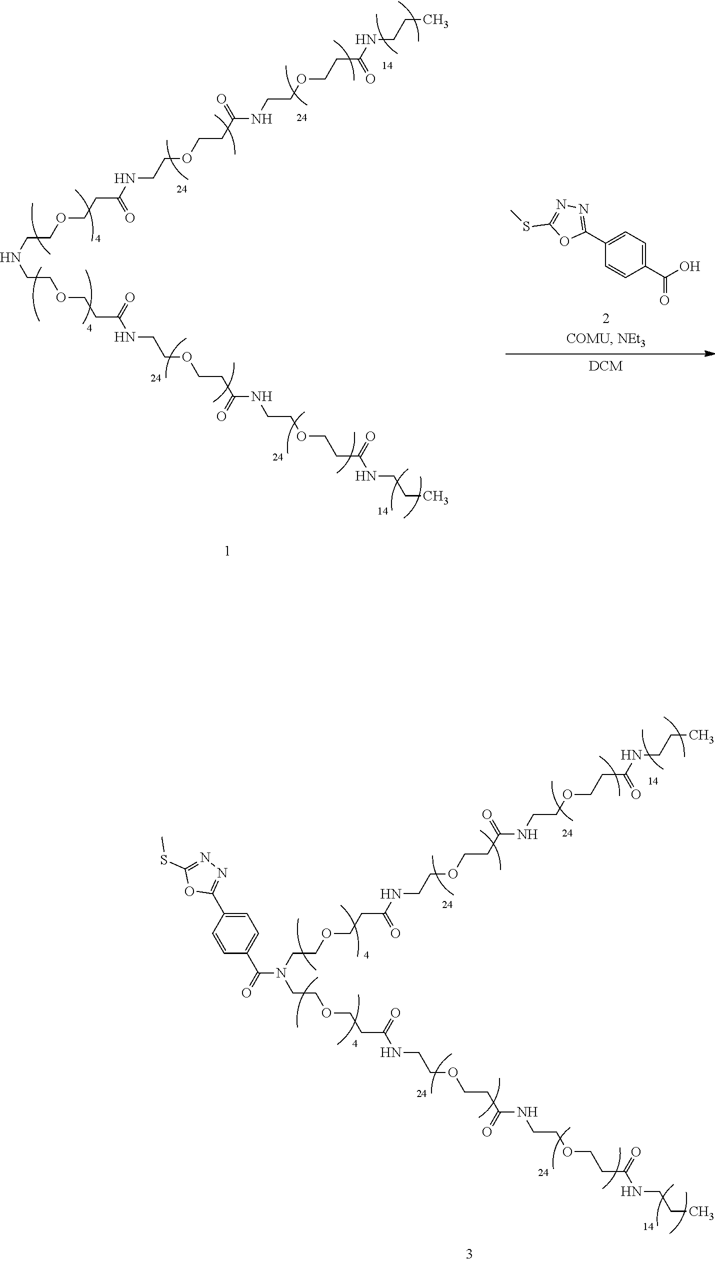

Compound 2 (11.2 mg, 0.047 mmol) and COMU (20 mg, 0.047 mmol) were dissolved in DCM (3 mL) and NEt₃ (16.7 μL, 0.12 mmol) was added. The resulting solution was stirred for 10 minutes. After 10 minutes, a solution of compound 1 (130 mg, 0.024 mmol, compound 8 from synthesis of LP246-p) in DCM (2 mL) was added to the solution of compound 2/COMU. The resulting solution was stirred for 1 hour and checked by LC-MS. The reaction mixture was quenched with 5 mL of water and stirred for 5 minutes. The layers were separated, and the organic layer was washed with 1 M HCl_{(aq)}(1×15 mL), sat. NaHCO_{3(aq)} (3×20 mL), sat. NaCl_{(aq)}(20 mL), dried over Na₂SO₄ and concentrated to yield a clear liquid. Crude product was purified by silica gel chromatography (4 g gold redisep column) 0-20% MeOH in DCM. Compound 3 eluted with 12% MeOH in DCM. Pure fractions of compound 3 were combined to yield 0.086 g (63.6% yield) as an off white solid.

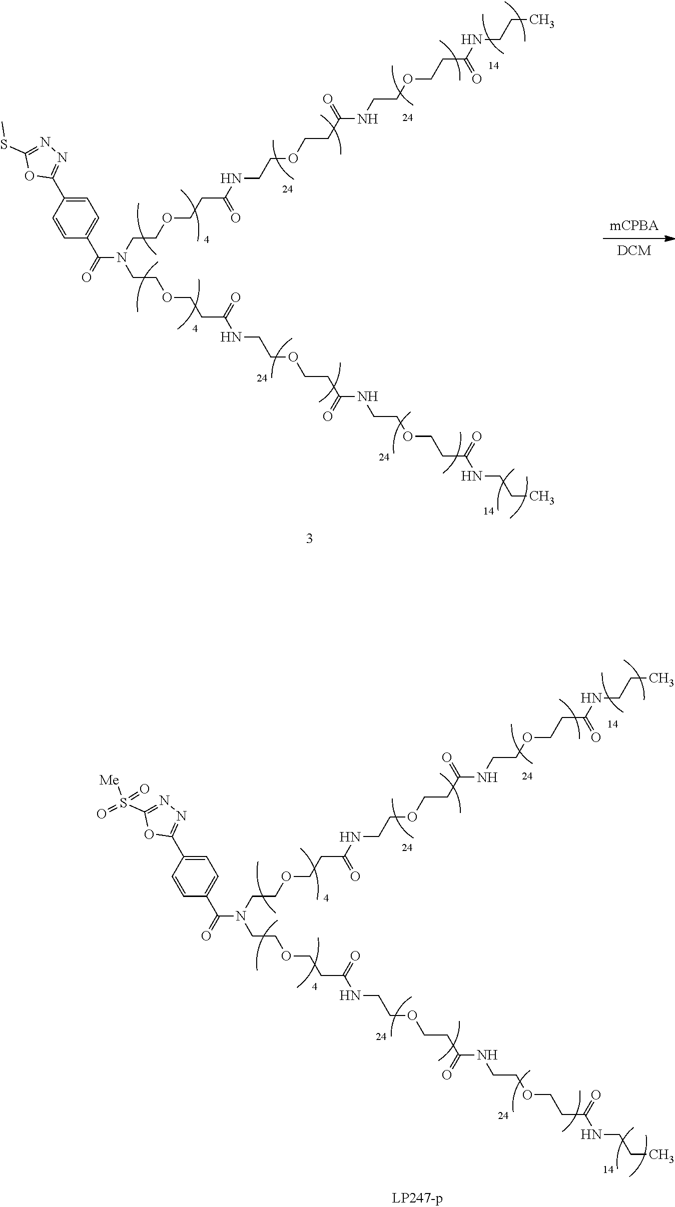

Compound 3 (0.086 g, 0.015 mmol) was dissolved in DCM (3 mL) and mCPBA (0.0131 g, 0.076 mmol) was added. The resulting solution was stirred overnight. The reaction mixture was concentrated and loaded directly onto a silica gel column. Crude product was purified by silica gel chromatography (4 g gold redisep column) 0-20% MeOH in DCM. LP247-p eluted with 12% MeOH in DCM. Pure fractions of LP247-p were combined to yield 0.041 mg (47.4% yield) as an off white solid.

### Synthesis of LP339-p

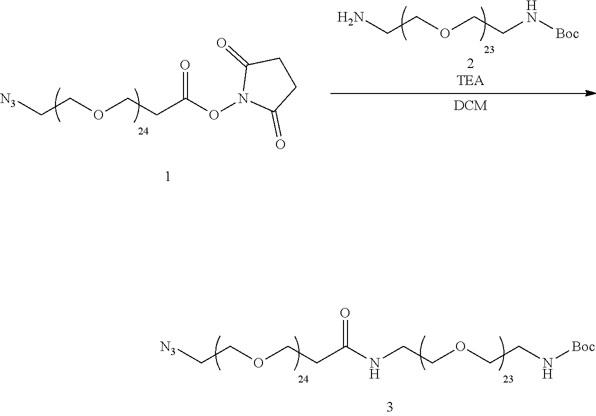

Boc-amido-PEG₂₃-amine 2 (8.00 g, 6.82 mmol) was dissolved in DCM (250 mL) and triethylamine (2.85 mL, 20.45 mmol) was added, followed by azido-PEG₂₄-NHS Ester 1 (9.95 g, 7.84 mmol). The reaction mixture was stirred at room temperature. After 2 hours no starting material remained as determined by LC-MS. The reaction mixture was concentrated and loaded directly onto a silica gel column for purification. The crude product was purified by silica gel chromatography 2% MeOH:98% DCM to 20% MeOH:80% DCM. Fractions containing the product were combined to yield 14.3 grams (90% yield) of compound 3 as a white solid.

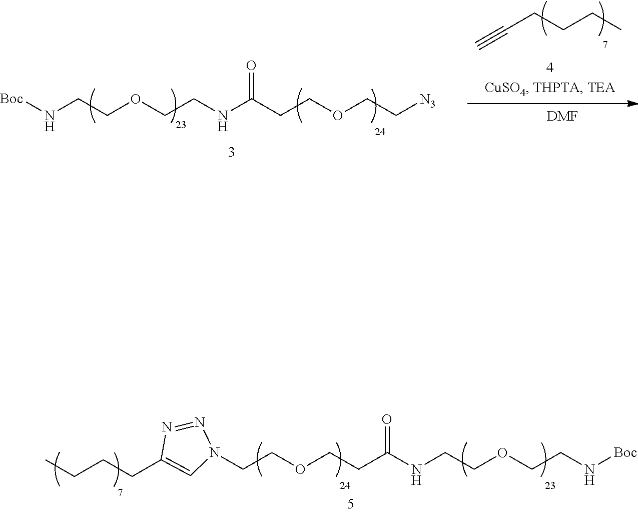

N-Boc-PEG₂₃-Amido-PEG₂₄-Azide 3 (10.0 g, 4.296 mmol), 1-octadecyne 4 (1.183 g, 4.726 mmol), copper sulfate pentahydrate (0.268 g, 1.074 mmol), tris((1-hydroxy-propyl-1H-1,2,3-triazole-4-yl)methyl)amine (THPTA) (0.653 g, 1.504 mmol), and sodium ascorbate (1.872 g, 9.451 mmol) were dissolved in DMF (500 mL) and triethylamine (0.290 mL, 2.148 mmol) was added. The reaction mixture was heated to 60° C. After 2 hours, no starting material was observed by LC-MS. The reaction mixture was concentrated, and the residue was diluted with dichloromethane and filtered through a fritted funnel. The filtrate was concentrated and loaded directly onto a silica gel column for purification. The crude product was purified by silica gel chromatography 0% MeOH:100% DCM to 20% MeOH:80% DCM. The product eluted at 8% MeOH/92% DCM. Pure fractions were combined to yield 9.5 g (86% yield) of compound 5 as a light yellow solid.

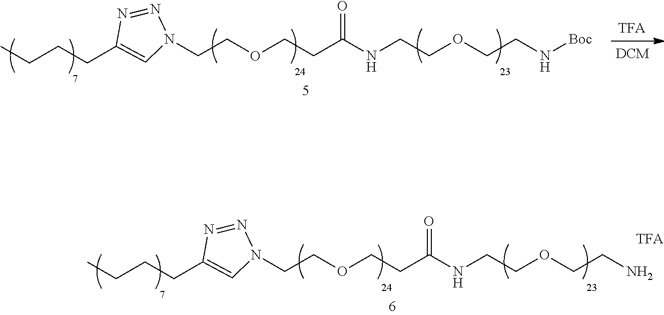

N-Boc-PEG₂₃-Amido-PEG₂₄-Triazole-C₁₆ 5 (0.358 g, 0.139 mmol) was dissolved in DCM (4 mL) and trifluoroacetic acid (0.9 mL, 11.8 mmol) was added. After 1 hour, no starting material was observed by LC-MS. The reaction mixture was concentrated and dried under vacuum for several hours to yield 0.325 mg (90.9% yield) of compound 6 as a light yellow solid. The product was used directly in the next reaction without further purification.

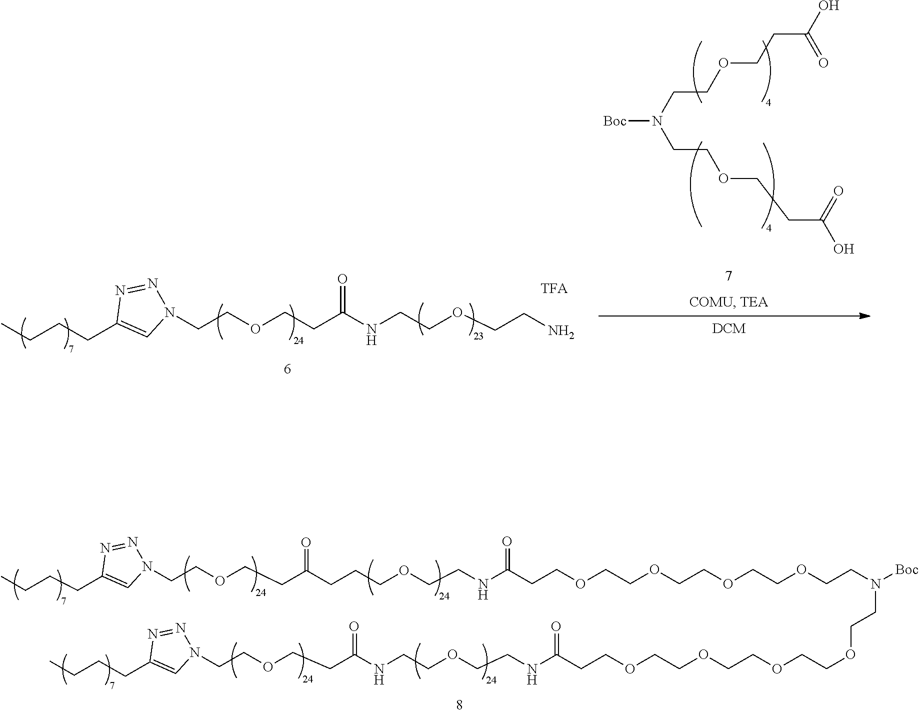

N-Boc-N-Bis-PEG₄-Acid 7 (0.0372 g, 0.061 mmol) and COMU (0.052 g, 0.121 mmol) were dissolved in DCM (5 mL) and TEA (0.395 mL, 2.84 mmol) was added. The resulting solution was stirred for 10 minutes. In a separate vial, a solution of the TFA salt of Amino-PEG₂₃-amido-PEG₂₄-triazole-C₁₆ 6 (0.325 g, 0.126 mmol) in DCM (5 mL) and TEA (0.5 mL, 3.60 mmol) was stirred. The solution ofN-Boc-N-Bis-PEG₄-Acid 7 was added to the solution of Amino-PEG₂₃-amido-PEG₂₄-triazole-C₁₆ 6. The reaction mixture was stirred overnight. The reaction mixture was concentrated and loaded directly onto a silica gel column for purification. The crude product was purified by silica gel chromatography 4% MeOH:96% DCM to 20% MeOH:80% DCM. Pure fractions were combined to yield 89 mg (26.5% yield) of compound 8 as a light yellow solid.

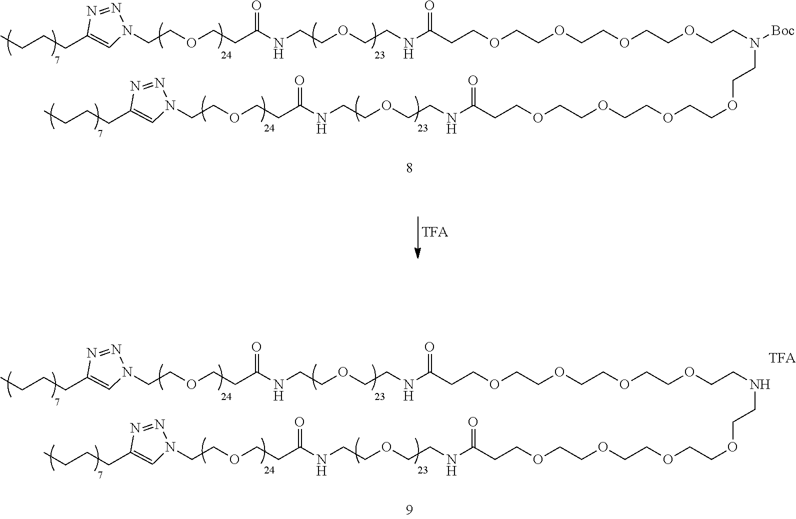

N-Boc-bis-PEG₄-Amido-PEG₂₃-amido-PEG₂₄-Triazole-C₁₆ 8 (5.9 g, 1.066 mmol) was dissolved in DCM (100 mL) and TFA (20 mL, 262.3 mmol) was added. After 2 hours, no starting material was observed by LC-MS. The reaction mixture was concentrated to afford compound 9 as a thick yellow liquid. Compound 9 was used directly in the next step without further purification.

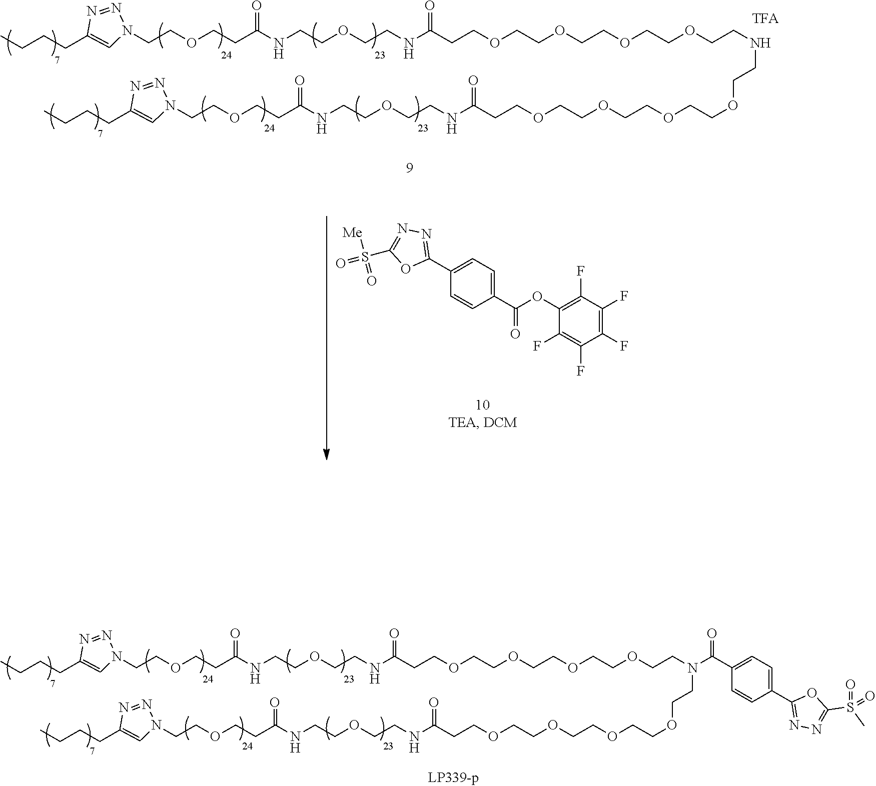

The TFA salt of amino-bis-PEG₄-Amido-PEG₂₃-amido-PEG₂₄-Triazole-C₁₆ 9 (5.89 g, 1.066 mmol) was dissolved in THE (100 mL) and TEA (1.5 mL, 10.66 mmol) and TFP-sulfone 10 (1.33 g, 3.20 mmol) was added. After 22 hours, LC-MS indicated 95% conversion to the product. The reaction mixture was concentrated, resuspended in toluene, and concentrated again prior to purification. The crude product was purified by silica gel chromatography 5% MeOH:95% DCM to 20% MeOH:80% DCM. The product eluted with 8% MeOH:92% DCM. Pure fractions were combined and yielded 3.000 g (49.5% yield) of LP339-p as a beige solid.

### Synthesis of LP340-p

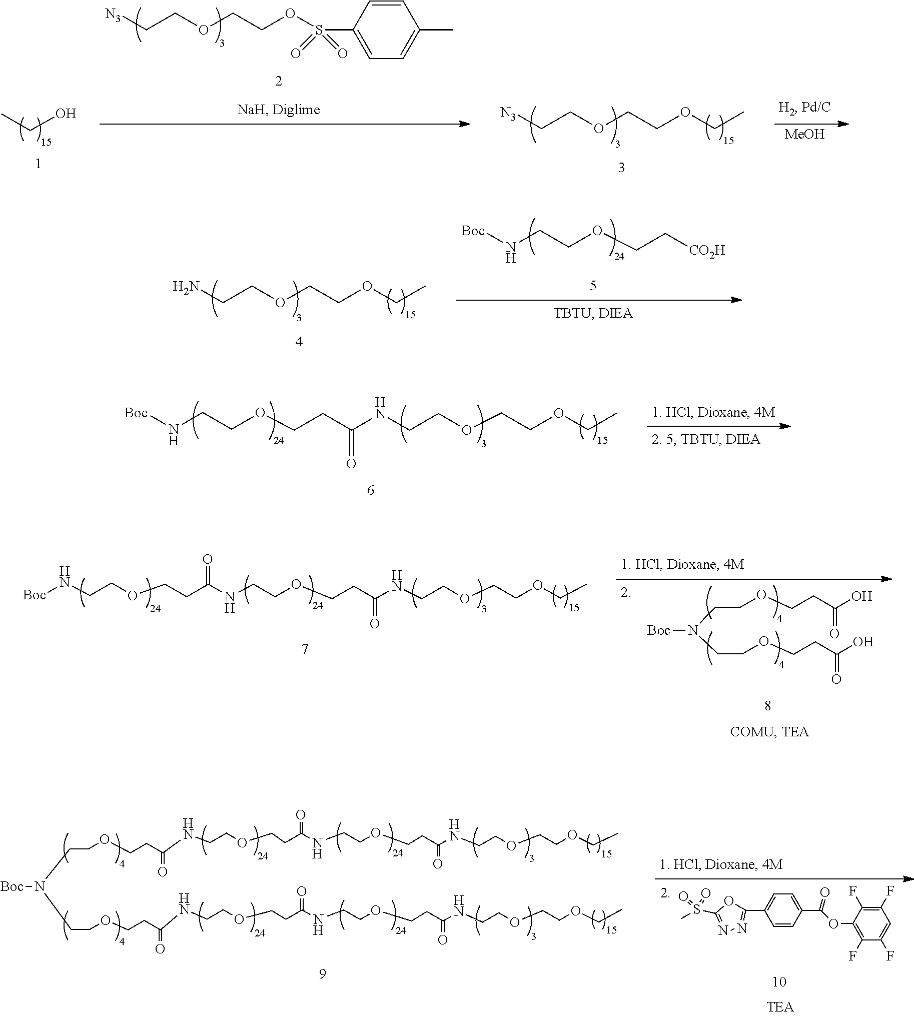

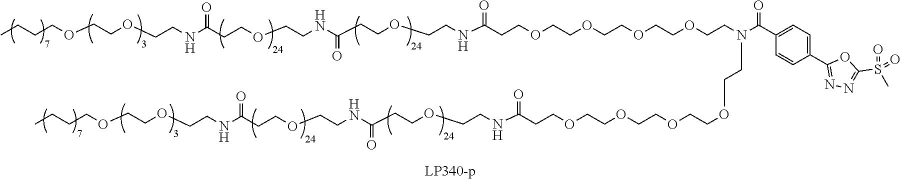

Sodium hydride, 60% dispersion in mineral oil (1.93 g, 48.21 mmol) was loaded in a dry 1 L round bottom flask, washed with MTBE and suspended in anhyd dioxane (200 mL). Hexadecanol 1 (11.2 g, 46.2 mmol) was added dry and stirred for 1 hour at 50° C. Peg3-tosylate 2 (15 g, 40.17 mmol) was added, and the reaction mixture was heated for 17 hours at 105° C. The reaction mixture was cooled in an ice bath and H₂O (125 mL) was added. The mixture was extracted with MTBE, and the organic layer was washed with H₂O, brine, and dried over Na₂SO₄. Compound 3 was purified on CombiFlash® using 220 g SiO₂ column, eluent: solvent A—hexane, solvent B—EtOAc; B=0-30%, 50 min. Yield, 11.1 g, 64%. Calculated MW 443.67 Found: MS (ES, pos): 444.67 [M+H]⁺, 461.5 [M+NH₄]⁺, 466.54 [M+Na]⁺.

Azide 3 (11.1 g, 25 mmol) was stirred with Pd/C₁₋₁₀% (1 g) in MeOH (70 mL) under a hydrogen atmosphere for 17 hours at 1 atmosphere. The reaction mixture was filtered, concentrated, and dried under vacuum. Compound 4 was purified on CombiFlash® using 80 g SiO₂ column, eluent: solvent A—DCM, solvent B—20% MeOH in DCM; B=0-50% in 50 min. Yield 4.66 g. Calculated: MW 417.7. Found: MS (ES, pos): 418.1 [M+H]⁺.

TBTU (4.8 g, 14.9 mmol) was added to a suspension of amine 4 (5.94 g, 14.2 mmol), Boc-(Peg 24)-acid 5 (16.95 g, 13.6 mmol), and DIEA (7.1 mL, 40.8 mmol) in DMF (100 mL). The reaction mixture was stirred for 3 hours, concentrated, and the residual DMF was removed by 3 co-evaporations with toluene. Crude compound 6 was dissolved in CHCl₃ (500 mL), washed with 1% HCl, NaHCO₃, brine, dried over Na₂SO₄, and used directly in the next step without further purification. Calculated: MW 1646.14. Found: MS (ES, pos): 1646.99 [M+H]⁺, 1664.99 [M+NH₄]⁺.

Compound 6 (10.14 g, 6.16 mmol) was stirred in a 4M HCl dioxane solution (45 mL) for 50 minutes. The reaction mixture was concentrated and the residue was dried by 2 co-evaporations with toluene. The resultant deprotected Peg-amine hydrochloride was dissolved in DMF (60 ml), then DIEA (4.29 mL, 24.6 mmol) and acid 5 (7.674 g, 6.157 mmol) were added, followed by TBTU (2.175 g, 6.77 mmol). The reaction mixture was stirred for 4 hours. The reaction mixture was concentrated and the residue was dried by 3 co-evaporations with toluene. The product, compound 7, was dissolved in CHCl₃ (500 mL), washed with 1% HCl, NaHCO₃, brine, and dried over Na₂SO₄. Compound 7 was used directly in the next step without further purification. Calculated: MW 2774.49. Found: MS (ES, pos): 1405.24 [M+2NH₄]²⁺, 1397.20 [M+H+Na]²⁺, 1388.67 [M+2H]²⁺.

Compound 7 (15.22 g, 5.49 mmol) was stirred in a 4M HCl dioxane solution (55 mL) for 50 minutes. The reaction mixture was concentrated and the residue was dried by 2 co-evaporations with toluene. The resultant deprotected Peg-amine hydrochloride was dissolved in DCM (100 mL). Boc-amino-bis(Peg4-acid) 8 (1.68 g, 2.74 mmol) was stirred in DCM (15 mL) with TEA (2.2 mL, 15.8 mmol) and COMU (2.47 g, 5.76 mmol) for 3 minutes, and then added to the solution of the deprotected Peg-amine hydrochloride. The reaction mixture was stirred for 3 hours and the solvent was removed. The residue was dissolved in chloroform (300 mL), washed with 1% HCl, NaHCO₃, brine, and dried over Na₂SO₄. Compound 9 was purified on CombiFlash® using SiO₂ column (220 g), eluent solvent A—DCM, solvent B—20% MeOH in DCM; B=0-100% in 50 min. Yield 9.75 g, (60%). Calculated: MW 5926.42. Found: MS (ES, pos): 1483.26 [M+3H+NH₄]⁴⁺, 1458.53.74 [M+4H]⁴⁺1186.91 [M+5H]⁺⁵.

Compound 9 (9.75 g, 1.644 mmol) was stirred in a 4M HCl dioxane solution (60 mL) for 50 minutes. The reaction mixture was concentrated and the residue was dried by 2 co-evaporations with toluene. The resultant amine hydrochloride was dissolved in THE (150 mL) and TEA was added (1.38 mL, 9.86 mmol), followed by sulfone-TFP ester 10 (1.711 g, 4.11 mmol). The reaction mixture was stirred for 16 hours, and the solvent was removed under vacuum. The residue was dissolved in chloroform (300 mL), washed with 1% HCl, brine, and dried over Na₂SO₄. LP340-p was purified on CombiFlash® using SiO₂ column (120 g), eluent solvent A—DCM, solvent B—20% MeOH in DCM; B=0-100% in 60 min. Yield 7.58 g, (75%). Calculated: MW 6077.54. Found: MS (ES, pos): 1534.03 [M+H+Na+2NH₄]⁴⁺, 1227.47 [M+2H+Na+2NH₄] ⁵⁺.

### Synthesis of LP357-p

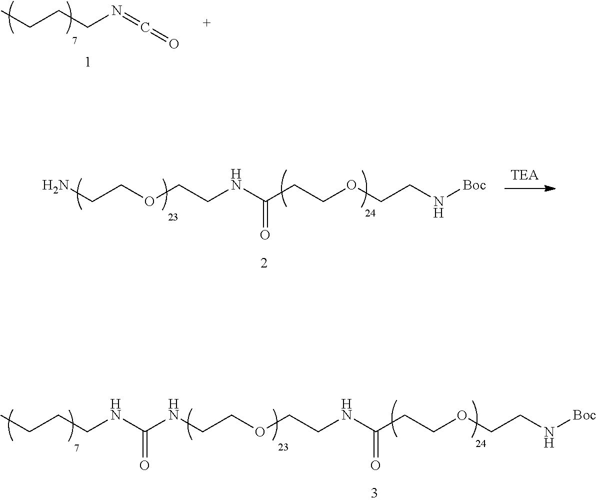

Boc-PEG₄₇-NH_{2 2} (1 g, 0.435 mmol, 1.0 equiv.) was dissolved in 20 mL DCM. Hexadecyl isocyanate 1 (140 mg, 0.522 mmol, 1.2 eqv.) and TEA (2.0 eqv.) were added and the reaction mixture was stirred at room temperature for 12 hours. DCM was removed and compound 3 0.967 g (86.5%) was purified via 24 g column purification using 0-20% MeOH/DCM as the mobile phase.

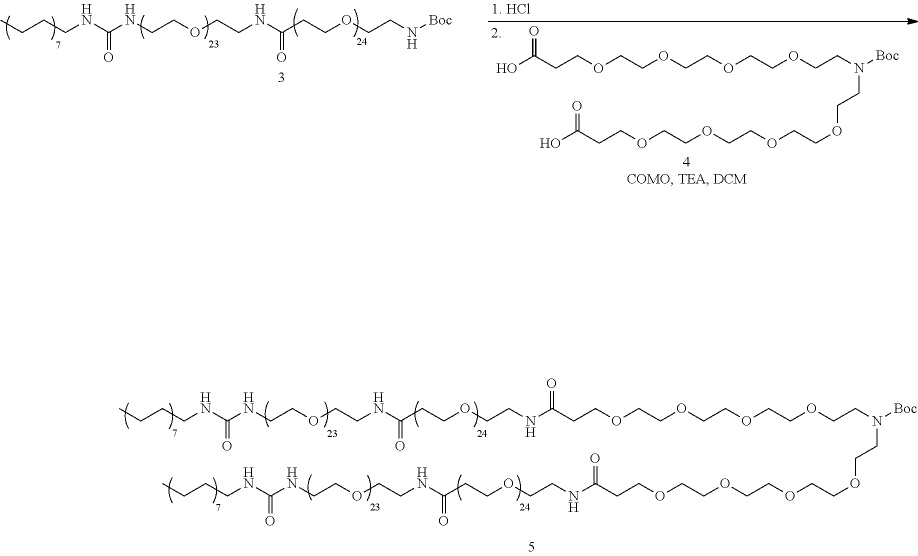

Compound 3 (0.967 g, 0.376 mmol) was dissolved in 15 mL of 4N HCl/dioxane and stirred at room temperature for 1 hour. The HCl/dioxane was removed and the resultant deprotected amine was dissolved in DCM. Compound 4 (110 mg, 0.179 mmol), COMU (169 mg, 0.394 mmol) and TEA (10.0 eqv.) were added and the reaction mixture was stirred at room temperature overnight. The solvent was removed under vacuum. Compound 5 (0.8 g, 80.9% yield) was purified by a 24 g column using 0-20% MeOH/DCM as the mobile phase.

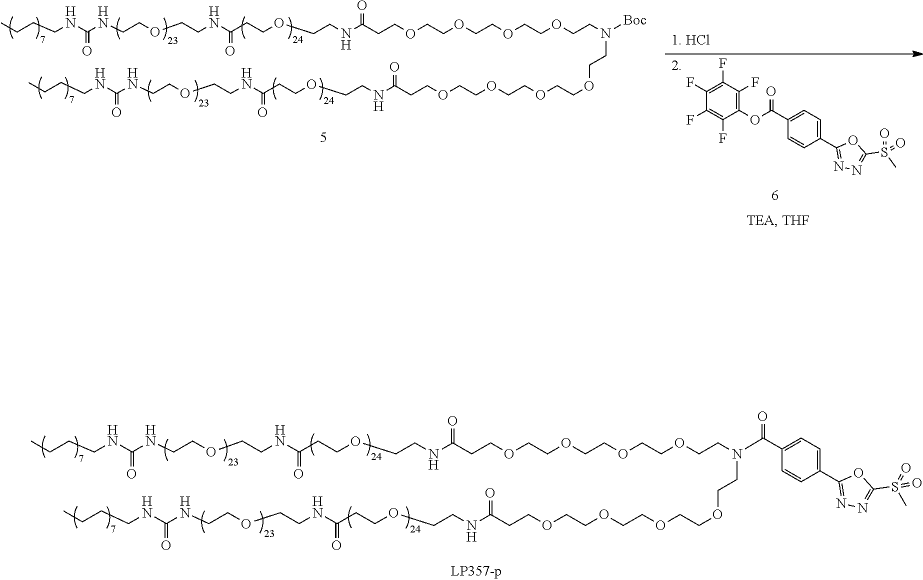

Compound 5 (0.95 g, 0.172 mmol) was dissolved in 15 mL of 4N HCl/Dioxane and stirred at room temperature for 1 hour. The HCl/dioxane was removed under vacuum. The resulting deprotected amine was dissolved in THF, then compound 6 (0.15 g, 0.345 mmol) and TEA (10.0 eqv.) were added. The reaction mixture was stirred at room temperature overnight. The solvent was removed under vacuum. LP357-p (0.6 g, 61%) was purified by a 24 g column using 0-20% MeOH/DCM as the mobile phase.

### Synthesis of LP358-p

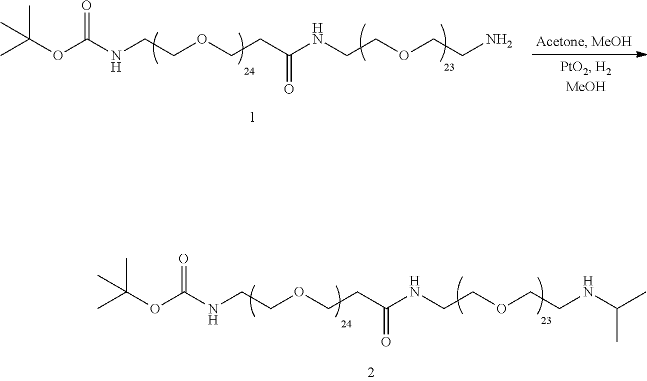

PtO₂ (0.3986 g) was added to a solution of compound 1 (4.00 g) in anhydrous McOH and acetone. The reaction mixture was stirred for two days under a hydrogen atmosphere. The platinum catalyst was filtered out using Celite® and silica. The solution was then concentrated under vacuum to afford compound 2 which was used directly in the next step without purification. Yield: 3.99 g.

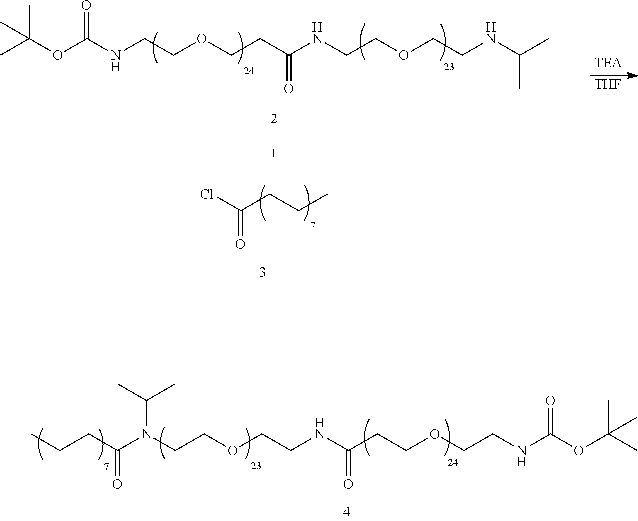

Compound 2 (4.07 g) was added to a solution of compound 3 (0.53 g) and TEA (0.53 g) in THF. The reaction mixture was stirred until full conversion of 2 was observed by LC-MS and/or TLC. The reaction mixture was quenched with MeOH. The crude product was purified on a CombiFlash® system via a DCM liquid-load (80 g column, DCM (A) to 20% MeOH (B) solvent system, gradient: 5% B to 100% B over 60 min). Compound 4 eluted at 25% B. Yield: 2.92 g.

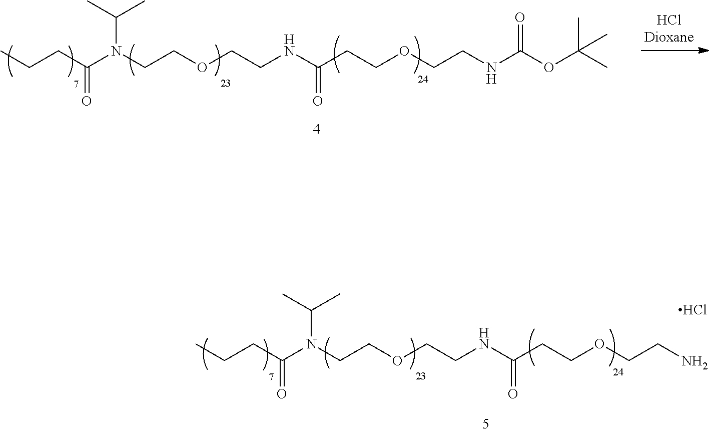

Compound 4 (2.92 g) was dissolved in a solution of HCl in Dioxane (4M) (24.9 mL) at room temperature. The reaction mixture was stirred until full conversion of compound 4 was observed via LCMS. The reaction mixture was concentrated under vacuum to afford compound 5 as a white powder. Compound 5 was used directly in the next step without further purification.

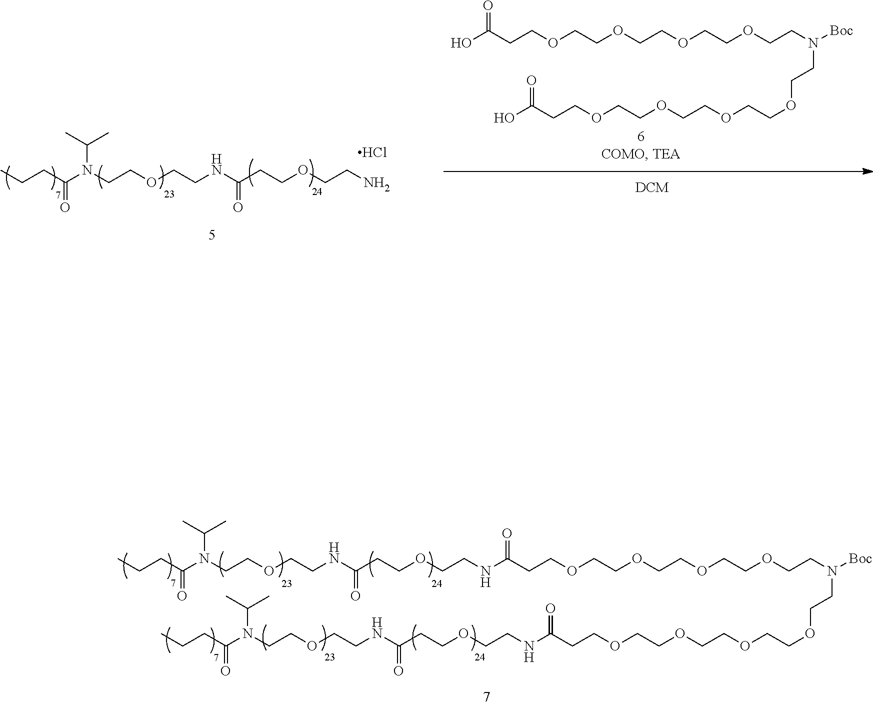

Compounds 6 (0.32 g) and 5 (2.81 g), COMU (1.07 g), and TEA (2.08 mL) were stirred in DCM at room temperature overnight. The pH was monitored to ensure that the HCl was neutralized and that the reaction mixture remained basic. The reaction mixture was washed with 1N HCl, saturated NaHCO₃,and brine, and the DCM was removed under vacuum. Compound 7 was purified via an 80 g column(Solvent system: DCM (A) and 20% McOH (B), gradient: 5% B for 5 min, 5% B to 100% B over 60 min). Compound 7 eluted at 45% B. Yield 2.26 g.

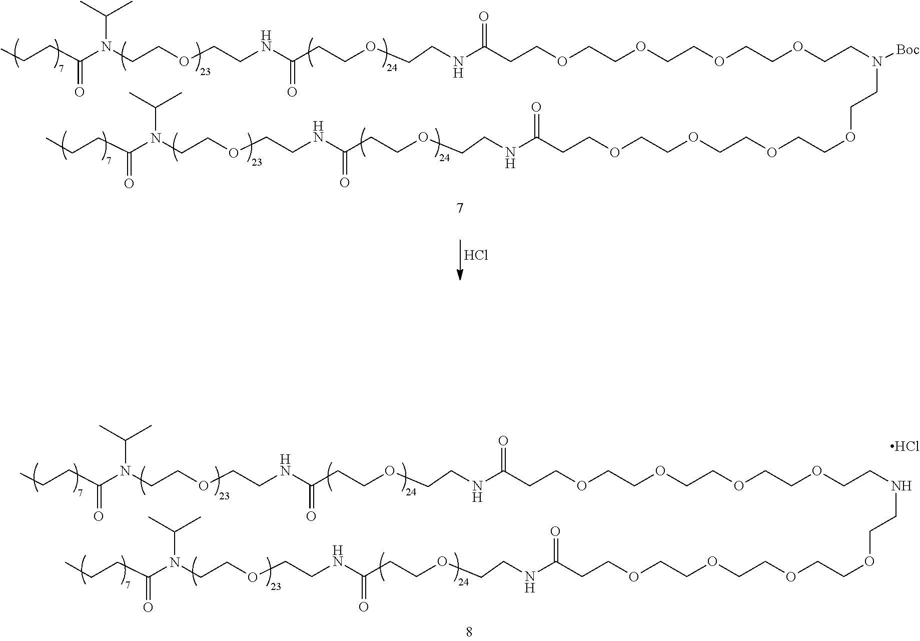

Compound 7 (2.26 g) was dissolved in a solution of HCl in Dioxane (4M) (25.5 mL) at room temperature. The reaction mixture was stirred until full conversion of compound 7 was observed via LCMS. The reaction mixture was concentrated under vacuum to afford compound 8 as a white powder. Compound 8 was used directly in the next step without further purification.

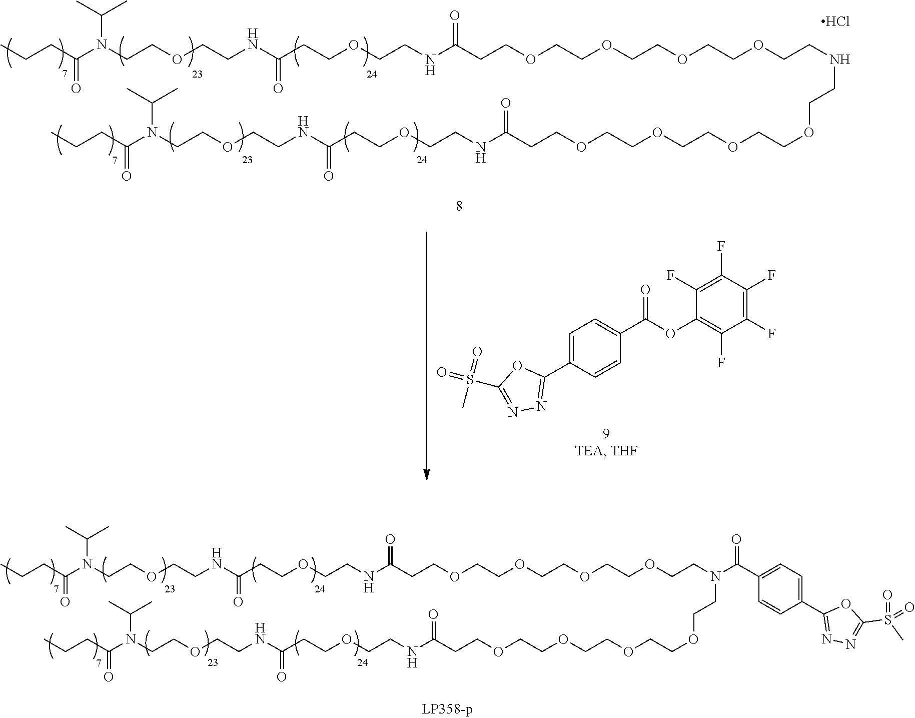

Compound 8 (2.22 g) and TEA (1.42 mL) were dissolved in 50 mL of anhydrous THF, and compound 9 (0.35 g) was added. The reaction mixture was stirred for 12 hours. The reaction mixture was concentrated and the crude LP358-p was purified by silica in two parts (12 and 24 gram columns) using two solvent systems (EtOAc/Hexanes followed by MeOH/DCM. 1st gradient (EtOAc/Hexanes): 0% B for 3 minutes, 0% B to 100% B over 10 min. 2nd gradient (DCM/MeOH): 5% B for 5 minutes, 15% B for 5 minutes, 15% B to 100% B over 20 min.). The product, LP358-p, eluted at 30% B during the second gradient. The sulfone reagent (i.e., compound 9) was recovered during the first gradient. Yield 1.92 g.

### Example 5. Conjugation of Linkers and Targeting Ligands to RNAi Agents

A. Conjugation of Activated Ester Linkers

The following procedure was used to conjugate linking groups having the structure of DBCO-NHS or L₁-L₁₀ as shown in Table 28 above to an RNAi agent with an amine-functionalized sense strand, such as C₆—NH₂, NH₂-C₆, or (NH₂-C₆)s, as shown in Table 28, above. An annealed RNAi Agent dried by lyophilization was dissolved in DMSO and 10% water (v/v %) at 25 mg/mL. Then 50-100 equivalents of TEA and 3 equivalents of activated ester linker were added to the solution. The solution was allowed to react for 1-2 hours, while monitored by RP-HPLC-MS (mobile phase A 100 mM HFIP, 14 mM TEA; mobile phase B: acetonitrile on an XBridge C18 column, Waters Corp.)

The product was then precipitated by adding 12 mL acetonitrile and 0.4 mL PBS and centrifuging the solid to a pellet. The pellet was then re-dissolved in 0.4 mL of 1×PBS and 12 mL of acetonitrile. The resulting pellet was dried on high vacuum for one hour.

B. Conjugation of Targeting Ligands to DBCO Linkers

The following procedure was used to link an azide-functionalized targeting ligand to a DBCO-functionalized linker such as DBCO-NHS, L₁ or L₂. The procedure selectively targets the DBCO portion of the linker for L₁ or L₂ such that the targeting ligand does not react with the propargyl group.

The solid RNAi pellet, comprising an RNAi agent with a covalently-linked DBCO moiety, was dissolved in 50/50 DMSO/water at 50 mg/mL. Then 1.5 equivalents of azide ligand per DBCO moiety were added. The reaction mixture was allowed to proceed for 30-60 minutes. The reaction mixture was monitored by RP-HPLC-MS (mobile phase A 100 mM HFIP, 14 mM TEA; mobile phase B: acetonitrile on an XBridge C₁₈ column, Waters Corp.) The product was precipitated by adding 12 mL acetonitrile, 0.4 mL PBS and the solid was centrifuged to a pellet. The pellet was re-dissolved in 0.4 mL 1×PBS and then 12 mL of acetonitrile was added. The pellet was dried on high vacuum.

C. Conjugation of Targeting Ligands to Propargyl Linkers

Either prior to or after annealing, the 5′ or 3′ tridentate alkyne functionalized sense strand is conjugated to the αvβ6 Integrin Ligands. The following example describes the conjugation of αvβ6 integrin ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5 H₂O) and 2M solution of sodium ascorbate were prepared in deionized water. A 75 mg/mL solution in DMSO of αvβ6 integrin ligand was made. In a 1.5 mL centrifuge tube containing tri-alkyne functionalized duplex (3 mg, 75 μL, 40 mg/mL in deionized water, approximately 15,000 g/mol), 25 μL of 1M Hepes pH 8.5 buffer is added. After vortexing, 35 μL of DMSO was added and the solution is vortexed. αvβ6 integrin ligand was added to the reaction (6 eq/duplex, 2 eq/alkyne, approximately 154) and the solution is vortexed. Using pH paper, pH was checked and confirmed to be pH approximately 8. In a separate 1.5 mL centrifuge tube, 50 μL of 0.5M THPTA was mixed with 10 uL of 0.5M Cu(II)SO₄·5 H₂O, vortexed, and incubated at room temp for 5 min. After 5 min, THPTA/Cu solution (7.2 μL, 6 eq 5:1 THPTA:Cu) was added to the reaction vial, and vortexed. Immediately afterwards, 2M ascorbate (5 μL, 50 eq per duplex, 16.7 per alkyne) was added to the reaction vial and vortexed. Once the reaction was complete (typically complete in 0.5-1h), the reaction mixture was immediately purified by non-denaturing anion exchange chromatography.

D. Conjugation of Targeting Ligands to Amine-Functionalized Sense Strand

The following procedure may be used to conjugate an activated ester-functionalized targeting ligand such as αvβ6 peptide 1, peptide 5 or peptide 6 to an amine functionalized RNAi agent comprising an amine, such as C₆—NH₂, NH₂-C₆, or (NH₂-C₆)s, as shown in Table 28.

An annealed, lyophilized RNAi agent was dissolved in DMSO and 10% water (v/v %) at 25 mg/mL. Then 50-100 equivalents TEA and three equivalents of activated ester targeting ligand were added to the mixture. The reaction mixture was allowed to stir for 1-2 hours while monitored by RP-HPLC-MS (mobile phase A: 100 mM HFIP, 14 mM TEA; mobile phase B: Acetonitrile; column:)(Bridge C₁₈). After the reaction mixture was complete, 12 mL of acetonitrile was added followed by 0.4 mL of PBS and then the mixture was centrifuged. The solid pellet was collected and dissolved in 0.4 mL of 1×PBS and then 12 mL of acetonitrile was added. The resulting pellet was collected and dried under vacuum for 1 hour.

### Example 6. Conjugation of PK/PD Modulator Precursors

Either prior to or after annealing and prior to or after conjugation of one or more targeting ligands, one or more PK/PD modulator precursors can be linked to the RNAi agents disclosed herein. The following describes the general conjugation process used to link PK/PD modulator precursors to the constructs set forth in the Examples depicted herein.

A. Conjugation of a Maleimide-Containing PK/PD Modulator

The following describes the general process used to link a maleimide-containing PK/PD modulator precursor to the (C₆—SS—C₆) or (6-SS-6) functionalized sense strand of an RNAi agent by undertaking a dithiothreitol reduction of disulfide followed by a thiol-Michael Addition of the respective maleimide-containing PK/PD modulator precursor: In a vial, functionalized sense strand was dissolved at 50 mg/mL in sterilized water. Then 20 equivalents of each of 0.1M Hepes pH 8.5 buffer and dithiothreitol were added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the maleimide-containing PK/PD modulator precursor was added at 1.5 equivalents. The mixture was allowed to react for 30 minutes. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

B. Conjugation of a sulfone-containing PK/PD modulator precursor

In a vial, functionalized sense strand was dissolved at 50 mg/mL in sterilized water.

Then 20 equivalents of each of 0.1M Hepes pH 8.5 buffer and dithiothreitol are added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the sulfone-containing PK/PD modulator precursor was added at 1.5 equivalents. The vial was purged with N₂, and heated to 40° C. while stirring. The mixture was allowed to react for one hour. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### C. Conjugation of an Azide-Containing PK/PD Modulator Precursor

One molar equivalent of TG-TBTA resin loaded with Cu(I) was weighed into a glass vial. The vial was purged with N₂ for 15 minutes. Then, functionalized sense strand was dissolved in a separate vial in sterilized water at a concentration of 100 mg/mL. Then two equivalents of the azide-containing PK/PD modulator precursor (50 mg/mL in DMF) is added to the vial. Then TEA, DMF and water are added until the final reaction conditions are 33 mM TEA, 60% DMF, and 20 mg/mL of the conjugated product. The solution was then transferred to the vial with resin via a syringe. The N₂ purge was removed and the vial was sealed and moved to a stir plate at 40° C. The mixture was allowed to react for 16 hours. The resin was filtered off using a 0.45 μm filter.

The product was purified using AEX purification (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile solid phase TSKgel-30; 1.5 cm×10 cm.) The acetonitrile was removed using a rotary evaporator, and desalted with a 3K spin column using 2×10 mL exchanges with sterilized water. The solid product was dried using lyophilization and stored for later use.

D. Conjugation of an Alkyne-Containing PK/PD Modulator Precursor

The following describes the general process used to link an activated alkyne-containing lipid PK/PD modulator precursor to the (C₆—SS—C₆) or (6-SS-6) functionalized sense strand of an RNAi agent by undertaking a dithiothreitol reduction of disulfide followed by addition to an alkyne-containing PK/PD modulator precursor: In a vial, 10 mg of siRNA comprising the (C₆—SS—C₆) or (6-SS-6) functionalized sense strand was dissolved at 50 mg/mL in sterilized water. Then 20 equivalents of each of 0.1M Hepes pH 8.5 buffer and dithiothreitol (1M in sterilized water) were added. The mixture was allowed to react for one hour, then purified on XBridge BEH C₄ Column using a mobile phase A of 100 mM HFIP, 14 mM, and TEA, and a mobile phase B of Acetonitrile using the following formula, wherein % B indicates the amount of mobile phase B while the remainder is mobile phase A.

| Time | % B |
|---|---|
| 0 | 3 |
| 8 | 70 |
| 10 | 90 |
| 11 | 90 |
| 11.1 | 3 |
| 13 | 3 |

The product was precipitated once by adding 12 mL of acetonitrile and 0.4 mL 1×PBS, and the resulting solid was centrifuged into a pellet. The pellet was re-dissolved in 0.4 mL 1×PBS and 12 mL of acetonitrile. The pellet was dried on high vacuum for one hour.

The pellet was brought up in a vial a 70/30 mixture of DMSO/water at a solids concentration of 30 mg/mL. Then, the alkyne-containing lipid PK/PD modulator precursor was added at 2 equivalents relative to siRNA. Then 10 equivalents of TEA was added. The vial was purged using N₂, and the reaction mixture was heated to 40° C. while stirring. The mixture was allowed to react for one hour. The product was purified using anion-exchange HPLC using a TSKgel-30 packed column, 1.5 cm×10 cm, using a mobile phase A of 25 mM TRIS pH=7.2, 1 mM EDTA, 50% Acetonitrile, and a mobile phase B of 25 mM TRIS pH=7.2, 1 mM EDTA, 500 mM NaBr, 50% Acetonitrile using the following formula, wherein % B indicates the amount of mobile phase B while the remainder is mobile phase A.

| Time | % B |
|---|---|
| 4 | 10 |
| 7 | 80 |
| 10.5 | 80 |
| 11 | 10 |
| 14 | 10 |

The fractions containing the product were collected, and acetonitrile was removed using a rotary evaporator. The product was desalted with a 3K spin column, using 2×10 mL exchanges with sterilized water. The product was then dried using lyophilization and stored for later use.

### Example 7. In Vivo Administration of RNAi Triggers Targeting Mstn in Mice

The following examples show the utility of the delivery platforms of the present invention. While the following examples include RNAi agents for the inhibition of myostatin, it is contemplated that the delivery platform may be used to knock down other genes of interest that are present in skeletal muscle cells.

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. RNAi agents used in this and following Examples have the structure as indicated in Table 25, below.

TABLE D
Duplexes used in the Following Examples.

| | | SEQ |
|---|---|---|
| Duplex | | ID |
| Name | Structure (5′ −> 3′) | NO |
| AD06326 | AS: usGfsusUfaCfagcaaGfaUfcAfuGfgCfsc | 1 |
| | SS: (NH2-C6)s(invAb)sggccaugaUfCfUfugcuguaacas(invAb)(C6-SS-C6)dT | 2 |
| AD06912 | AS: usGfsusUfaCfagcaaGfaUfcAfuGfgCfsc | 3 |
| | SS: (Alk-cyHex)s(invAb)sggccaugaUfCfUfugcuguaacas(invAb)(C6-SS-C6)dT | 4 |
| AD06916 | AS: usGfsusUfaCfagcaaGfaUfcAfuGfgCfsc | 5 |
| | SS: (NH2-C6)s(invAb)sggccaugaUfCfUfugcuguaacas(invAb)s(C6-SS-C6)dT | 6 |
| AD06569 | AS: cPrpusGfsusUfaCfagcaaGfaUfcAfuGfaCfsc | 7 |
| | SS: (NH2-C6)s(invAb)sggucaugaUfCfUfugcuguaacas(invAb)(C6-SS-C6)dT | 8 |

Wherein in Table D above a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue (see Table 28); dT represents 2′-deoxythymidine-3′-phosphate; (C₆—SS—C₆) see Table 28; (NH₂-C₆)s see Table 28; (Alk-cyHex)s see Table 28.

On Study Day 1, mice were injected with either isotonic saline (vehicle control) or 3 mg/kg (mpk) of RNAi agent formulated in isotonic saline according to the following dosing Groups:

TABLE 29
Dosing Groups for mice of Example 7.

| Group | RNAi agent and dose | Dosing Regimen |
|---|---|---|
| 1. | Vehicle Control | Single Injection on Day 1 |
| 2. | Compound 45b-(L4)-Mstn(AD06326)-PEG48 + C22 | Single Injection on Day 1 |
| 3. | Compound 45b-Mstn(AD06912)-PEG48 + C22 | Single Injection on Day 1 |
| 4. | Compound 45b-(L4)-Mstn(AD06916)-PEG48 + C22 | Single Injection on Day 1 |

The RNAi agents in Example 7 were synthesized having nucleotide sequences directed to target the MSTN gene, and groups 1 and 3 included a functionalized amine reactive group (NH₂-C₆) at the 5′ terminal end of the sense strand to facilitate conjugation to L₄ which could then be conjugated to the small molecule targeting ligand Compound 45b. Group 2 included an (Alk-cyHex)s (see Table 28 for structure details) which was conjugated to the small molecule targeting ligand Compound 45b. The myostatin RNAi agents further included a PEG48+C₂₂ PK/PD modulator, which was linked to the 3′ end of the sense strand.

Four (4) mice were dosed in each Group (n=4). Mice were sacrificed on study day 22, and total myostatin mRNA was isolated from the triceps. Triceps were harvested from right front limb. Each sample was snap-frozen in percellys tubes and stored in a −80° C. freezer until assays were completed. Relative MSTN expression was determined by qPCR TaqMan Assay on muscle tissue. Average relative myostatin expression in harvested tissue is shown in Table 30 below.

TABLE 30
Average relative MSTN expression from
triceps samples for mice of Example 7.

| | Day 22 | | |
|---|---|---|---|
| Group | Relative Expression. | Low Error | High Error |
| Group 1 (Isotonic Saline) | 1.000 | 0.246 | 0.327 |
| Group 2 (AD06326) | 0.296 | 0.074 | 0.099 |
| Group 3 (AD06912) | 0.253 | 0.067 | 0.092 |
| Group 4 (AD06916) | 0.295 | 0.052 | 0.063 |

### Example 8. In Vivo Administration of RNAi triggers Targeting MSTN in Cynomolgus Monkeys

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. On Study Day 1, cynomolgus macaque (Macaca fascicularis) primates (referred to herein as “cynos”) were injected with either isotonic saline (vehicle control) or 10 mg/kg (mpk) of RNAi agent formulated in isotonic saline according to the following dosing Groups:

TABLE 31
Dosing Groups for cynos of Example 8.

| Group | RNAi agent and dose | Dosing Regimen |
|---|---|---|
| 1. | Isotonic Saline | Single Injection |
| | | on Day 1 |
| 4. | Compound 45b-(L4)-Mstn(AD06569)-PEG48 + C22 | Single Injection |
| | | on Day 1 |
| 5. | Compound 45b-(L4)-Mstn(AD06569)-Bis(PEG47 + C22) | Single Injection |
| | | on Day 1 |

The RNAi agents in Example 8 were synthesized having nucleotide sequences directed to target the MSTN gene, and included a functionalized amine reactive group (NH₂-C₆) at the 5′ terminal end of the sense strand to facilitate conjugation to the small molecule targeting ligand Compound 45b. The myostatin RNAi agents further included a disulfide functional group (C₆—SS—C₆) at the 3′ terminal end of the sense strand to facilitate conjugation to a PK/PD modulator. Various PK/PD modulators were linked to the 3′ end of the sense strand, as specified in Table 31, above.

Three (3) cynos were dosed in each Group (n=3). Serum samples were taken on days −14, −7, and day 1 (pre-dose). Monkeys were then administered according to the respective Groups as set forth in Table 31. Serum was then collected on days 8, 15, 22, and 29. An ELISA assay was performed on serum samples to determine the amount of cyno myostatin in serum. Average myostatin in serum samples is shown in Table 32 below.

TABLE 32
Average cyno myostatin protein in serum of Example 8, normalized to Day 1.

| | Day 8 | | Day 15 | | Day 22 | | Day 29 | |
|---|---|---|---|---|---|---|---|---|
| | Avg | Std Dev | Avg | Std Dev | Avg | Std Dev | Avg | Std Dev |
| Group ID | Mstatin | (+/−) | Mstatin | (+/−) | Mstatin | (+/−) | Mstatin | (+/−) |
| Group 1, Animal 1 | 1.038 | 0.005 | 0.914 | 0.009 | 1.090 | 0.019 | 0.845 | 0.002 |
| Group 1, Animal 2 | 0.908 | 0.005 | 0.795 | 0.042 | 0.828 | 0.006 | 0.862 | 0.002 |
| Group 1, Animal 3 | 0.936 | 0.018 | 0.866 | 0.060 | 0.739 | 0.025 | 0.813 | 0.012 |
| Group 4, Animal 1 | 0.838 | 0.021 | 0.809 | 0.037 | 0.689 | 0.010 | 0.511 | 0.022 |
| Group 4, Animal 2 | 0.917 | 0.019 | 0.931 | 0.060 | 0.702 | 0.038 | 0.651 | 0.014 |
| Group 4, Animal 3 | 0.751 | 0.031 | 0.798 | 0.008 | 0.832 | 0.021 | 0.685 | 0.031 |
| Group 5, Animal 1 | 0.793 | 0.001 | 0.552 | 0.012 | 0.550 | 0.001 | 0.365 | 0.007 |
| Group 5, Animal 2 | 0.723 | 0.014 | 0.611 | 0.020 | 0.433 | 0.011 | 0.423 | 0.006 |
| Group 5, Animal 3 | 0.744 | 0.001 | 0.588 | 0.017 | 0.552 | 0.013 | 0.458 | 0.024 |

### Example 9. In Vivo Administration of RNAi triggers Targeting Mstn in Mice

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. On Study Days 1, 8, 15, and 43 mice were injected with either isotonic saline (vehicle control) or 3 mg/kg (mpk) of RNAi agent formulated in isotonic saline according to the following dosing Groups:

TABLE 33
Dosing Groups for mice of Example 9.

| Group | RNAi agent and dose | Dosing Regimen |
|---|---|---|
| 1. | Isotonic Saline (IV) | Injections on days 1, |
| | | 8, 15 and 43 |
| 3. | Compound 45b-Linker 4-AD06326- | Injections on days 1, |
| | PEG48 + C22 (IV) | 8, 15 and 43 |
| 4. | Compound 45b-Linker 4-AD06326- | Injections on days 1, |
| | PEG48 + C18 (IV) | 8, 15 and 43 |
| 5. | Compound 45b-Linker 4-AD06326- | Injections on days 1, |
| | bis(PEG47 + C22) (IV) | 8, 15 and 43 |
| 6. | Isotonic Saline (SQ) | Injections on days 1, |
| | | 8, 15 and 43 |
| 7. | Compound 45b-Linker 4-AD06326- | Injections on days 1, |
| | PEG48 + C22 (SQ) | 8, 15 and 43 |
| 8. | Compound 45b-Linker 4-AD06326- | Injections on days 1, |
| | PEG48 + C18 (SQ) | 8, 15 and 43 |
| 9. | Compound 45b-Linker 4-AD06326- | Injections on days 1, |
| | bis(PEG47 + C22) (SQ) | 8, 15 and 43 |
| 10. | Compound 45b-Linker 4-AD06326- | Injections on days 1, |
| | PEG48 + C22 (SQ) | 8, 15 and 43 |

As shown in Table 33 above, some groups were dosed intravenously, while others were dosed subcutaneously, as indicated. The RNAi agents in Example 8 were synthesized having nucleotide sequences directed to target the MS TN gene, and included a functionalized amine reactive group (NH₂-C₆) at the 5′ terminal end of the sense strand to facilitate conjugation to the small molecule targeting ligand Compound 45a. The myostatin RNAi agents further included a PEG 40K (4-arm), PEG₄₈+C₁₈, PEG₄₈+C₂₂, or bis(PEG₄₇+C₂₂) PK/PD modulator, which was linked to the 3′ end of the sense strand.

Four (4) mice were dosed in each Group (n=4). Mice were bled on days 1, 8, 15, 21, 29, 36, 43, 50, 57 and 64, and the serum was isolated. An ELISA assay was performed to determined the relative amount of myostatin in each serum sample. Average myostatin in serum samples is shown in Table 34 below.

TABLE 34
Average relative mouse myostatin protein in serum of Example 9.

| | Day 8 | | Day 15 | | Day 21 | | Day 29 | | Day 36 | | Day 43 | | Day 50 | | Day 57 | | Day 64 | |
|---|---|---|---|---|---|---|---|---|---|---|---|---|---|---|---|---|---|---|
| | | Std. | | Std. | | Std. | | Std. | | Std. | | Std. | | Std. | | Std. | | Std. |
| Group | Avg | Dev. | Avg | Dev. | Avg | Dev. | Avg | Dev. | Avg | Dev. | Avg | Dev | Avg | Dev. | Avg | Dev. | Avg | Dev. |
| 1 | 1.018 | 0.066 | 0.901 | 0.138 | 1.074 | 0.122 | 1.033 | 0.059 | 1.010 | 0.134 | 1.021 | 0.092 | 1.029 | 0.120 | 0.930 | 0.079 | 0.899 | 0.116 |
| 3 | 0.613 | 0.132 | 0.381 | 0.052 | 0.284 | 0.036 | 0.254 | 0.053 | 0.250 | 0.059 | 0.302 | 0.085 | 0.282 | 0.030 | 0.210 | 0.028 | 0.215 | 0.042 |
| 4 | 0.588 | 0.062 | 0.384 | 0.051 | 0.248 | 0.017 | 0.186 | 0.023 | 0.205 | 0.018 | 0.214 | 0.021 | 0.232 | 0.024 | 0.208 | 0.033 | 0.171 | 0.039 |
| 5 | 0.518 | 0.060 | 0.320 | 0.027 | 0.205 | 0.014 | 0.163 | 0.011 | 0.179 | 0.024 | 0.193 | 0.027 | 0.225 | 0.029 | 0.170 | 0.035 | 0.162 | 0.027 |
| 6 | 1.018 | 0.072 | 1.049 | 0.104 | 1.085 | 0.068 | 0.923 | 0.090 | 0.947 | 0.080 | 1.015 | 0.060 | 1.088 | 0.050 | 0.884 | 0.048 | 0.876 | 0.085 |
| 7 | 0.480 | 0.027 | 0.205 | 0.017 | 0.124 | 0.003 | 0.093 | 0.010 | 0.093 | 0.004 | 0.095 | 0.007 | 0.100 | 0.008 | 0.080 | 0.002 | 0.074 | 0.005 |
| 8 | 0.567 | 0.082 | 0.301 | 0.028 | 0.217 | 0.016 | 0.180 | 0.026 | 0.167 | 0.024 | 0.177 | 0.025 | 0.209 | 0.021 | 0.169 | 0.024 | 0.151 | 0.026 |
| 9 | 0.606 | 0.042 | 0.353 | 0.033 | 0.287 | 0.014 | 0.228 | 0.023 | 0.237 | 0.018 | 0.251 | 0.028 | 0.259 | 0.009 | 0.213 | 0.012 | 0.195 | 0.015 |
| 10 | 0.490 | 0.082 | 0.284 | 0.038 | 0.208 | 0.028 | 0.180 | 0.018 | 0.178 | 0.031 | 0.215 | 0.032 | 0.200 | 0.030 | 0.177 | 0.024 | 0.164 | 0.012 |

### Example 10. In Vivo Administration of RNAi triggers Targeting Mstn in Mice

Myostatin RNAi agents that included a sense strand and an antisense strand were synthesized according to phosphoramidite technology on solid phase in accordance with general procedures known in the art and commonly used in oligonucleotide synthesis, as set forth in Example 1 herein. On Study Day 1, mice were injected with either isotonic saline (vehicle control) or 3 mg/kg (mpk) of RNAi agent formulated in isotonic saline according to the following dosing Groups:

TABLE 35
Dosing Groups for mice of Example 10.

| Group | RNAi agent and dose | Dosing Regimen |
|---|---|---|
| 1. | Vehicle Control | Single Injection on Day 1 |
| 3. | SM6.1(DBCO)-AD06326-PEG23 + C22 | Single Injection on Day 1 |
| 4. | SM6.1(DBCO)-AD06326-bis(PEG23 + C14) | Single Injection on Day 1 |
| 5. | SM6.1(DBCO)-AD06326-bis(PEG23 + C18) | Single Injection on Day 1 |
| 6. | SM6.1(DBCO)-AD06326-bis(PEG23 + C22) | Single Injection on Day 1 |
| 7. | SM6.1(DBCO)-AD06326-bis(PEG23 + CLS) | Single Injection on Day 1 |
| 8. | SM6.1(DBCO)-AD06326-bis(PEG47 + C22) | Single Injection on Day 1 |
| 9. | SM6.1(DBCO)-AD06326-tris(PEG23 + C22) | Single Injection on Day 1 |
| 10. | SM6.1(DBCO)-AD06326-tris(PEG23 + CLS) | Single Injection on Day 1 |

The RNAi agents in Example 10 were synthesized having nucleotide sequences directed to target the MSTN gene, and included a functionalized amine reactive group (NH₂-C₆) at the 5′ terminal end of the sense strand to facilitate conjugation to the small molecule targeting ligand Structure 6.1. The myostatin RNAi agents further included a variety of PK/PD modulators as shown in Table 35, which was linked to the 3′ end of the sense strand using the method described in Example 1.

Four (4) mice were dosed in each Group (n=4). Mice were bled and serum was then collected on days 8, 15, and 22. An ELISA assay was performed on serum samples to determine the amount of mouse myostatin in serum. Average myostatin in serum samples is shown in Table 36 below.

TABLE 36
Average relative MSTN in serum for the groups of Example 10.

| | | Day 8 | | Day 15 | | Day 22 | |
|---|---|---|---|---|---|---|---|
| | | | Std. | | Std | | Std. |
| Group | Description | Avg. | Dev. | Avg. | Dev. | Avg. | Dev. |
| 1 | Vehicle Control | 1.133 | 0.083 | 1.292 | 0.068 | 1.213 | 0.093 |
| 3 | SM6.1(DBCO)-AD06326- | 0.587 | 0.061 | 0.499 | 0.058 | 0.429 | 0.058 |
| | PEG23 + C22 | | | | | | |
| 4 | SM6.1(DBCO)-AD06326- | 0.747 | 0.055 | 0.650 | 0.027 | 0.602 | 0.081 |
| | bis(PEG23 + C14) | | | | | | |
| 5 | SM6.1(DBCO)-AD06326- | 0.595 | 0.018 | 0.453 | 0.045 | 0.459 | 0.017 |
| | bis(PEG23 + C18) | | | | | | |
| 6 | SM6.1(DBCO)-AD06326- | 0.513 | 0.024 | 0.475 | 0.028 | 0.407 | 0.030 |
| | bis(PEG23 + C22) | | | | | | |
| 7 | SM6.1(DBCO)-AD06326- | 0.809 | 0.043 | 0.833 | 0.078 | 0.798 | 0.060 |
| | bis(PEG23 + CLS) | | | | | | |
| 8 | SM6.1(DBCO)-AD06326- | 0.385 | 0.040 | 0.391 | 0.059 | 0.375 | 0.050 |
| | bis(PEG47 + C22) | | | | | | |
| 9 | SM6.1(DBCO)-AD06326- | 0.752 | 0.104 | 0.809 | 0.108 | 0.765 | 0.083 |
| | tris(PEG23 + C22) | | | | | | |
| 10 | SM6.1(DBCO)-AD06326- | 0.781 | 0.065 | 0.954 | 0.041 | 0.900 | 0.052 |
| | tris(PEG23 + CLS) | | | | | | |

### Example 11. In Vivo Administration of RNAi triggers Targeting MSTN in Mice

On Study Day 1, mice were injected with either isotonic saline (vehicle control) or 2 mg/kg (mpk) of RNAi agent formulated in isotonic saline according to the following dosing Groups, wherein AD06569 has the structure shown in Table D above:

TABLE 37
Dosing Groups for mice of Example 11.

| Group | RNAi agent and dose | Dosing Regimen |
|---|---|---|
| 1 | Saline | Single Injection on Day 1 |
| 2 | 2 mpk AD06569-LP1 | Single Injection on Day 1 |
| 3 | 2 mpk SM45-(L4)-AD06569-LP1b | Single Injection on Day 1 |
| 8 | 2 mpk SM45-(L4)-AD06569-LP28b | Single Injection on Day 1 |
| 9 | 2 mpk SM45-(L4)-AD06569-LP56b | Single Injection on Day 1 |
| 10 | 2 mpk SM45-(L4)-AD06569-LP89b | Single Injection on Day 1 |

The RNAi agent AD06569 was synthesized having a nucleotide sequence targeted to the MST IV gene, and included a functionalized amine reactive group (NH₂-C₆) at the 5′ terminal end of the sense strand to facilitate conjugation to the small molecule targeting ligand Compound 45b. The RNAi agent was also synthesized having a (C₆—SS—C₆) group on the 3′ end, to facilitate conjugation to a lipid PK/PD modulator.

Groups 3 and 8-10 comprise an αvβ6 integrin ligand SM45 conjugated to the 5′ end of the sense strand using linker 4 according to procedures described in Example 5, above. Each of groups 2, 3 and 8-10 comprise a lipid PK/PD modulator, with structures as shown in supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right front limb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 38 below.

TABLE 38
Average relative MSTN expression from serum for mice of Example 11.

| | | Day 8 | | Day 15 | | Day 22 | |
|---|---|---|---|---|---|---|---|
| | | | Std | | Std | | Std |
| Group | | Avg | Dev | Avg | Dev | Avg | Dev |
| 1. | Saline | 0.795 | 0.126 | 0.893 | 0.108 | 0.940 | 0.058 |
| 2. | AD06569-LP1b | 0.620 | 0.074 | 0.435 | 0.028 | 0.417 | 0.033 |
| 3. | SM45-(L4)-AD06569-LP1b | 0.296 | 0.040 | 0.170 | 0.023 | 0.173 | 0.020 |
| 8. | SM45-(L4)-AD06569-LP28b | 0.394 | 0.047 | 0.249 | 0.037 | 0.241 | 0.044 |
| 9. | SM45-(L4)-AD06569-LP56b | 0.499 | 0.066 | 0.315 | 0.037 | 0.292 | 0.034 |
| 10. | SM45-(L4)-AD06569-LP89 | 0.310 | 0.034 | 0.218 | 0.056 | 0.173 | 0.025 |

Tissue collected from the gastrocnemius and triceps was used in a TaqMan assay to determine the relative amounts of MSTN in those tissues. Table 39, below, shows the results of the assay.

TABLE 39
Relative Expression in Triceps and Gastrocnemius
in dosing groups of Example 11.

| | | Triceps | | | Gastrocnemius | | |
|---|---|---|---|---|---|---|---|
| | | Rel. | Low | High | Rel. | Low | High |
| Group | | Exp. | Error | Error | Exp. | Error | Error |
| 1. | Saline | 1.000 | 0.105 | 0.118 | 1.000 | 0.092 | 0.101 |
| 2. | AD06569-LP1b | 0.171 | 0.067 | 0.110 | 0.175 | 0.041 | 0.053 |
| 3. | SM45-(L4)-AD06569-LP1b | 0.089 | 0.009 | 0.010 | 0.063 | 0.007 | 0.008 |
| 8. | SM45-(L4)-AD06569-LP28b | 0.110 | 0.022 | 0.028 | 0.088 | 0.014 | 0.016 |
| 9. | SM45-(L4)-AD06569-LP56b | 0.164 | 0.036 | 0.047 | 0.147 | 0.039 | 0.053 |
| 10. | SM45-(L4)-AD06569-LP89 | 0.101 | 0.021 | 0.027 | 0.096 | 0.012 | 0.014 |

### Example 12. In Vivo Administration of RNAi triggers Targeting MSTN in Mice

On Study Day 1, mice were injected with either isotonic saline (vehicle control) or 2 mg/kg (mpk) of RNAi agent formulated in isotonic saline according to the following dosing Groups, wherein AD06569 has the structure shown in Table D above:

TABLE 40
Dosing Groups for mice of Example 12.

| Group | RNAi agent and dose | Dosing Regimen |
|---|---|---|
| 1 | Saline | Single Injection on Day 1 |
| 2 | 2 mpk SM45-(L4)-AD06569-LP1 | Single Injection on Day 1 |
| 3 | 2 mpk SM45-(L4)-AD06569-LP90 | Single Injection on Day 1 |
| 4 | 2 mpk SM45-(L4)-AD06569-LP94 | Single Injection on Day 1 |
| 5 | 2 mpk SM45-(L4)-AD06569-LP93 | Single Injection on Day 1 |
| 6 | 2 mpk SM45-(L4)-AD06569-LP92 | Single Injection on Day 1 |
| 7 | 2 mpk SM45-(L4)-AD06569-LP91 | Single Injection on Day 1 |
| 8 | 2 mpk SM45-(L4)-AD06569-LP95 | Single Injection on Day 1 |
| 9 | 2 mpk SM45-(L4)-AD06569-LP5 | Single Injection on Day 1 |
| 10 | 2 mpk SM45-(L4)-AD06569-LP87 | Single Injection on Day 1 |

The RNAi agent AD06569 was synthesized having a nucleotide sequence targeted to the MSTN gene, and included a functionalized amine reactive group (NH₂-C₆) at the 5′ terminal end of the sense strand to facilitate conjugation to the small molecule targeting ligand Compound 45b. The RNAi agent was also synthesized having a (C₆—SS—C₆) group on the 3′ end, to facilitate conjugation to a lipid PK/PD modulator.

Groups 2-10 comprise an αvβ6 integrin ligand SM45 conjugated to the 5′ end of the sense strand using linker 4 according to procedures described in Example 5, above. Each of groups 2-10 comprise a lipid PK/PD modulator, with structures as shown supra, conjugated to the 3′ end of the sense strand according to procedures described in Example 6, above.

Four (4) mice were dosed in each Group (n=4). Mice were bled and serum was collected on days 1, 8, 15 and 22. Mice were sacrificed on study day 22, and total myostatin mRNA was isolated from the gastrocnemius and triceps. Triceps were harvested from right frontlimb. Each sample was snap-frozen in percellys tubes and stored in a −80° C. freezer until assays were completed. Relative MSTN expression was determined by ELISA assay on mouse myostatin in serum. Average relative myostatin expression in serum is shown in Table 41 below.

TABLE 41
Average relative MSTN expression from serum for mice of Example 12.

| | | Day 8 | | Day 15 | | Day 22 | |
|---|---|---|---|---|---|---|---|
| | | | Std | | Std | | Std |
| Group | | Avg | Dev | Avg | Dev | Avg | Dev |
| 1. | Saline | 1.005 | 0.133 | 1.075 | 0.162 | 1.120 | 0.163 |
| 2. | SM45-(L4)-AD06569-LP1b | 0.238 | 0.029 | 0.160 | 0.025 | 0.149 | 0.022 |
| 3. | SM45-(L4)-AD06569-LP90b | 0.642 | 0.062 | 0.612 | 0.041 | 0.614 | 0.021 |
| 4. | SM45-(L4)-AD06569-LP94b | 0.479 | 0.057 | 0.300 | 0.021 | 0.331 | 0.033 |
| 5. | SM45-(L4)-AD06569-LP93b | 0.353 | 0.028 | 0.241 | 0.040 | 0.207 | 0.022 |
| 6. | SM45-(L4)-AD06569-LP92b | 0.385 | 0.021 | 0.279 | 0.044 | 0.242 | 0.009 |
| 7. | SM45-(L4)-AD06569-LP91b | 0.240 | 0.024 | 0.177 | 0.020 | 0.154 | 0.029 |
| 8. | SM45-(L4)-AD06569-LP95b | 0.467 | 0.022 | 0.347 | 0.046 | 0.315 | 0.097 |
| 9. | SM45-(L4)-AD06569-LP5b | 0.447 | 0.032 | 0.411 | 0.031 | 0.371 | 0.030 |
| 10. | SM45-(L4)-AD06569-LP87b | 0.445 | 0.064 | 0.399 | 0.077 | 0.376 | 0.061 |

Tissue collected from the gastrocnemius and triceps was used in a TaqMan assay to determine the relative amounts of MSTN in those tissues. Table 42, below, shows the results of the assay.

TABLE 42
Relative Expression in Triceps and Gastrocnemius
in dosing groups of Example 12.

| | | Triceps | | | Gastrocnemius | | |
|---|---|---|---|---|---|---|---|
| | | Rel. | Low | High | Rel. | Low | High |
| Group | | Exp. | Error | Error | Exp. | Error | Error |
| 1. | Saline | 1.000 | 0.237 | 0.311 | 1.000 | 0.070 | 0.075 |
| 2. | SM45-(L4)-AD06569-LP1b | 0.069 | 0.016 | 0.020 | 0.103 | 0.013 | 0.015 |
| 3. | SM45-(L4)-AD06569-LP90b | 0.490 | 0.035 | 0.037 | 0.477 | 0.041 | 0.045 |
| 4. | SM45-(L4)-AD06569-LP94b | 0.189 | 0.041 | 0.052 | 0.215 | 0.036 | 0.043 |
| 5. | SM45-(L4)-AD06569-LP93b | 0.097 | 0.013 | 0.016 | 0.142 | 0.006 | 0.006 |
| 6. | SM45-(L4)-AD06569-LP92b | 0.120 | 0.014 | 0.016 | 0.175 | 0.034 | 0.042 |
| 7. | SM45-(L4)-AD06569-LP91b | 0.072 | 0.015 | 0.020 | 0.100 | 0.019 | 0.024 |
| 8. | SM45-(L4)-AD06569-LP95b | 0.271 | 0.030 | 0.034 | 0.277 | 0.047 | 0.057 |
| 9. | SM45-(L4)-AD06569-LP5b | 0.191 | 0.028 | 0.032 | 0.249 | 0.025 | 0.028 |
| 10. | SM45-(L4)-AD06569-LP87b | 0.165 | 0.021 | 0.024 | 0.209 | 0.025 | 0.029 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A delivery platform for inhibiting expression of a gene expressed in skeletal muscle cells comprising:
(a) An RNAi agent comprising:
(i) An antisense strand comprising 17-49 nucleotides wherein at least 15 nucleotides are complementary to the mRNA sequence of a gene that is expressed in skeletal muscle cells
(ii) A sense strand that is 16-49 nucleotides in length that is at least partially complementary to the antisense strand;
(b) A targeting ligand with affinity for a receptor present on the surface of a skeletal muscle cell; and
(c) A PK/PD modulator;
wherein the RNAi agent is covalently linked to the targeting ligand and to the PK/PD modulator.

2. The delivery platform of claim 1, wherein the targeting ligand has affinity for an integrin receptor.

3. The delivery platform of claim 1, wherein the targeting ligand has affinity for the αvβ6 integrin receptor.

4. The delivery platform of claim 1, wherein the targeting ligand is of the formula: 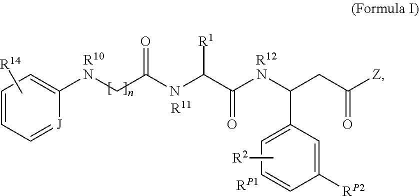
or a pharmaceutically acceptable salt thereof,
wherein,
n is an integer from 0 to 7;
J is C—H or N;
Z is OR¹³, N(R¹³)₂ or SR¹³;
R¹ is H, optionally substituted C₁-C₆ alkyl, OH, COOH, CON(R⁵)₂, OR⁶, or R¹ comprises a cargo molecule, wherein each R⁵ is independently H or C₁-C₆ alkyl, and R⁶ is H or C₁-C₆ alkyl;
R², R^{1′} and R² are each independently H, halo, optionally substituted cycloalkylene, optionally substituted arylene, optionally substituted heterocycloalkylene, or optionally substituted heteroarylene, or R², R^{1′} and R² may comprise a cargo molecule;
R¹⁰ is H or optionally substituted alkyl;
R¹¹ is H or optionally substituted alkyl, or R¹¹ and R¹ together with the atoms to which they are attached form an optionally substituted heterocycle;
R¹² is H or optionally substituted alkyl;
each R¹³ is independently H, optionally substituted alkyl, or R¹³ comprises a cargo molecule;
R¹⁴ is optionally substituted alkyl; and
wherein at least one of R¹, R², R¹³, R^{P1} and R² comprises the antisense strand.

5. The delivery platform of claim 4, wherein the targeting ligand is selected from the group consisting of: 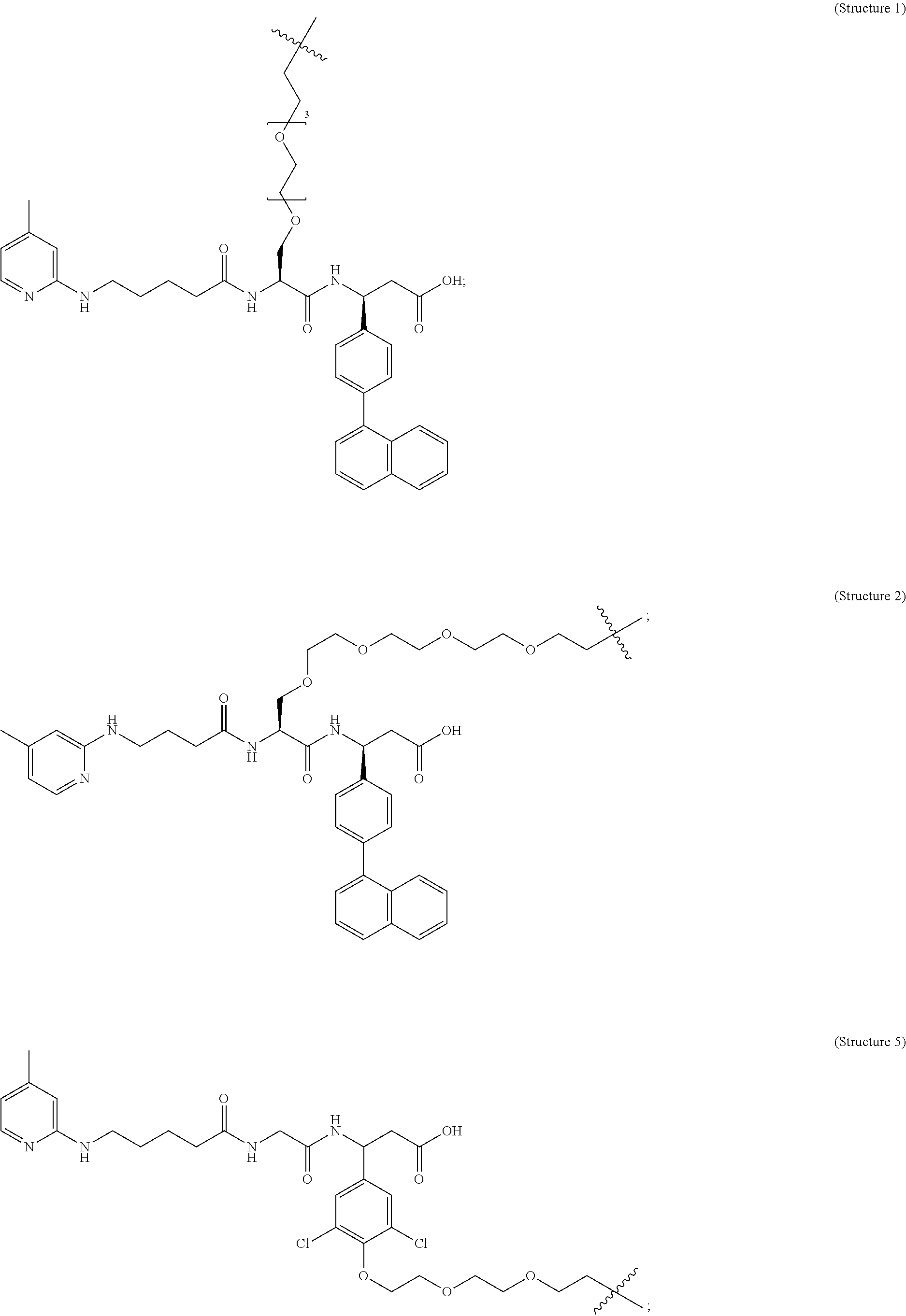 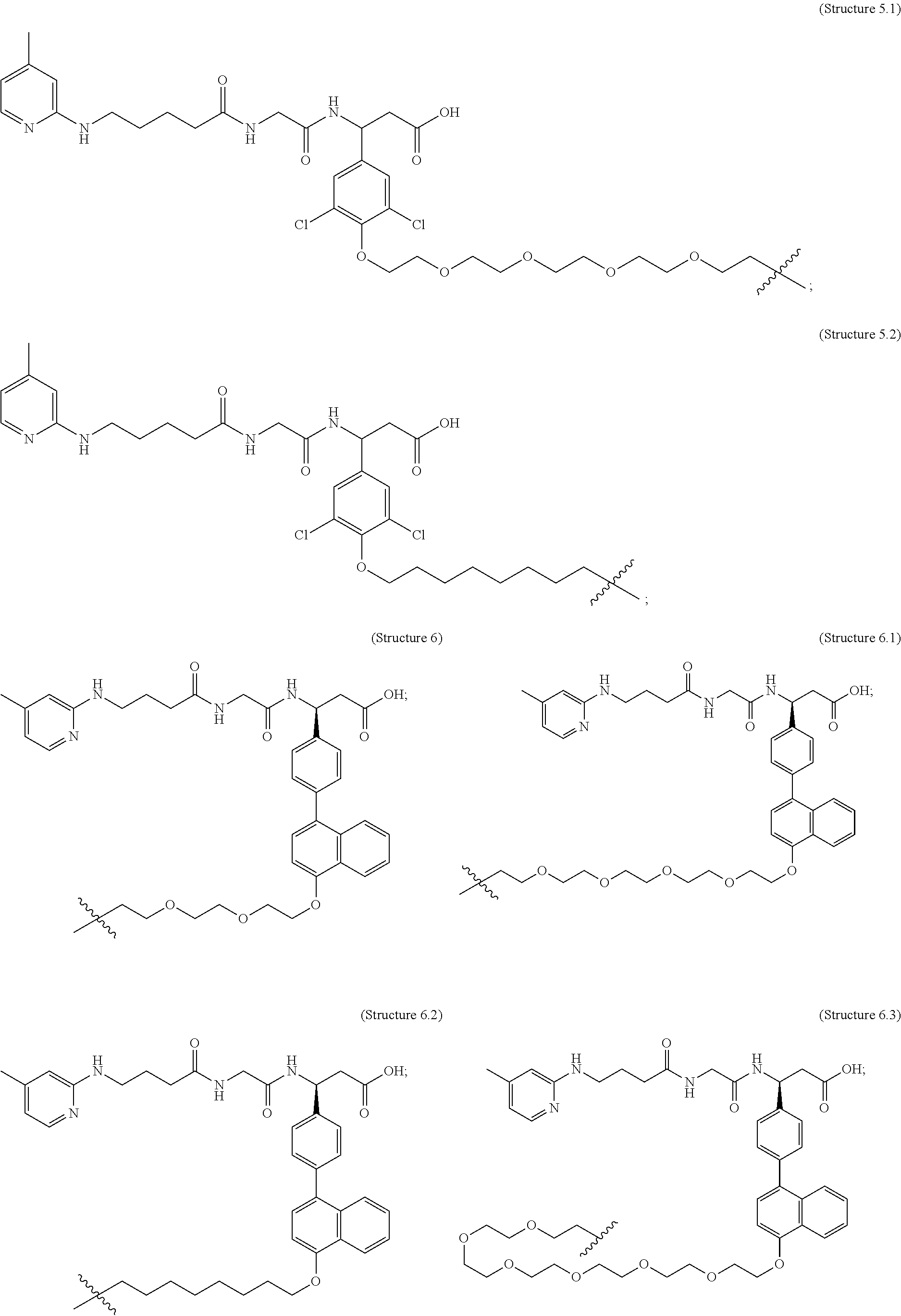 [SMILES: *CCOCCOCCOCCOCCOc1c(Cl)cc(C(CC(=O)O)NC(=O)CNC(=O)CCCCNc2cc(C)ccn2)cc1Cl ; *CCCCCCCCOc1c(Cl)cc(C(CC(=O)O)NC(=O)CNC(=O)CCCCNc2cc(C)ccn2)cc1Cl ; *CCOCCOCCOc1ccc(-c2ccc([C@H](CC(=O)O)NC(=O)CNC(=O)CCCNc3cc(C)ccn3)cc2)c2ccccc12 ; *CCOCCOCCOCCOCCOc1ccc(-c2ccc([C@H](CC(=O)O)NC(=O)CNC(=O)CCCNc3cc(C)ccn3)cc2)c2ccccc12 ; *CCCCCCCCOc1ccc(-c2ccc([C@H](CC(=O)O)NC(=O)CNC(=O)CCCNc3cc(C)ccn3)cc2)c2ccccc12 ; *CCOCCOCCOCCOCCOCCOCCOc1ccc(-c2ccc([C@H](CC(=O)O)NC(=O)CNC(=O)CCCNc3cc(C)ccn3)cc2)c2ccccc12] 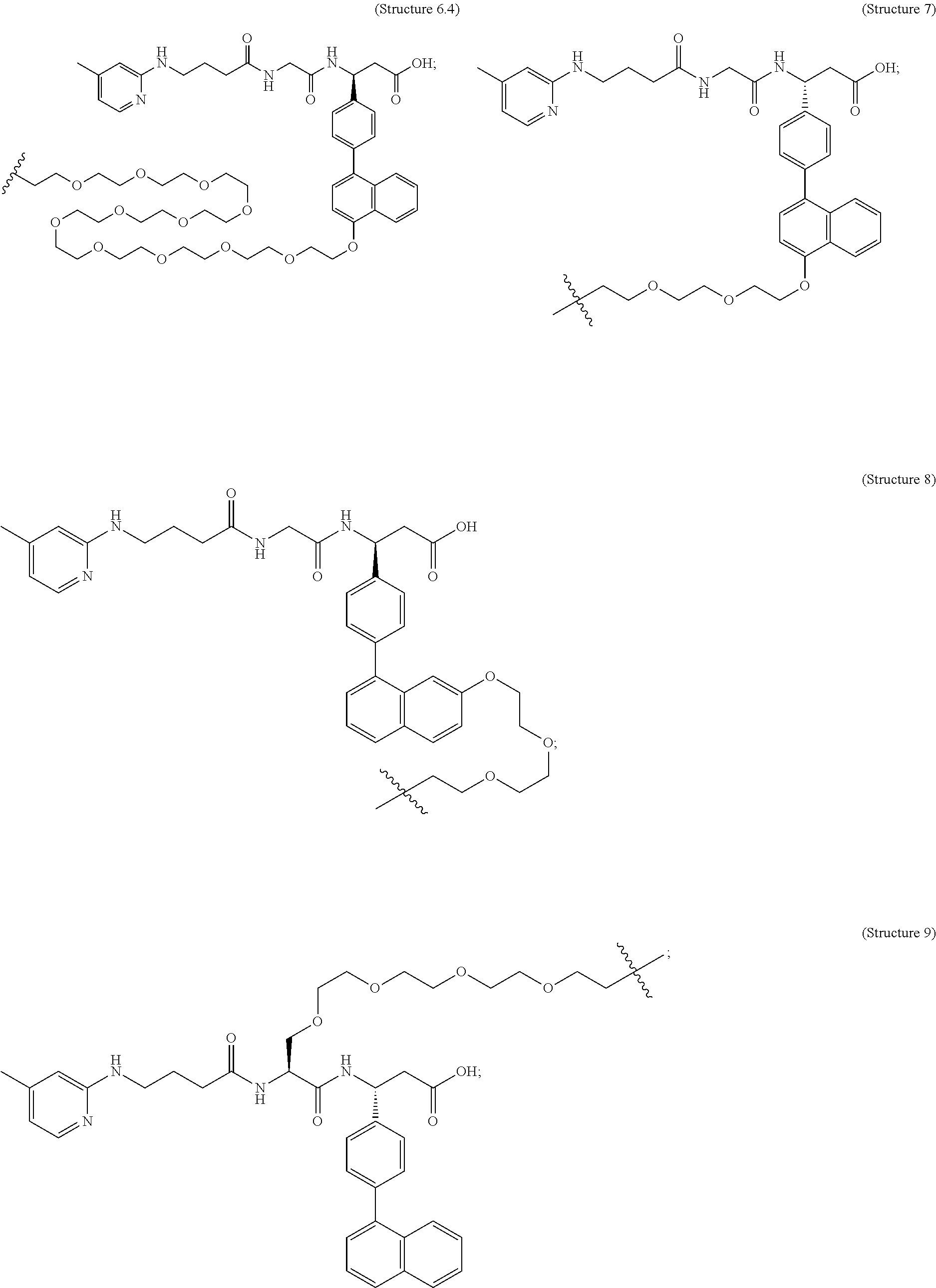 [SMILES: *CCOCCOCCOCCOCCOCCOCCOCCOCCOCCOCCOCCOc1ccc(-c2ccc([C@H](CC(=O)O)NC(=O)CNC(=O)CCCNc3cc(C)ccn3)cc2)c2ccccc12 ; *CCOCCOCCOc1ccc(-c2ccc([C@@H](CC(=O)O)NC(=O)CNC(=O)CCCNc3cc(C)ccn3)cc2)c2ccccc12 ; *CCOCCOCCOc1ccc2cccc(-c3ccc([C@H](CC(=O)O)NC(=O)CNC(=O)CCCNc4cc(C)ccn4)cc3)c2c1 ; *CCOCCOCCOCCOC[C@H](NC(=O)CCCNc1cc(C)ccn1)C(=O)N[C@H](CC(=O)O)c1ccc(-c2cccc3ccccc23)cc1] 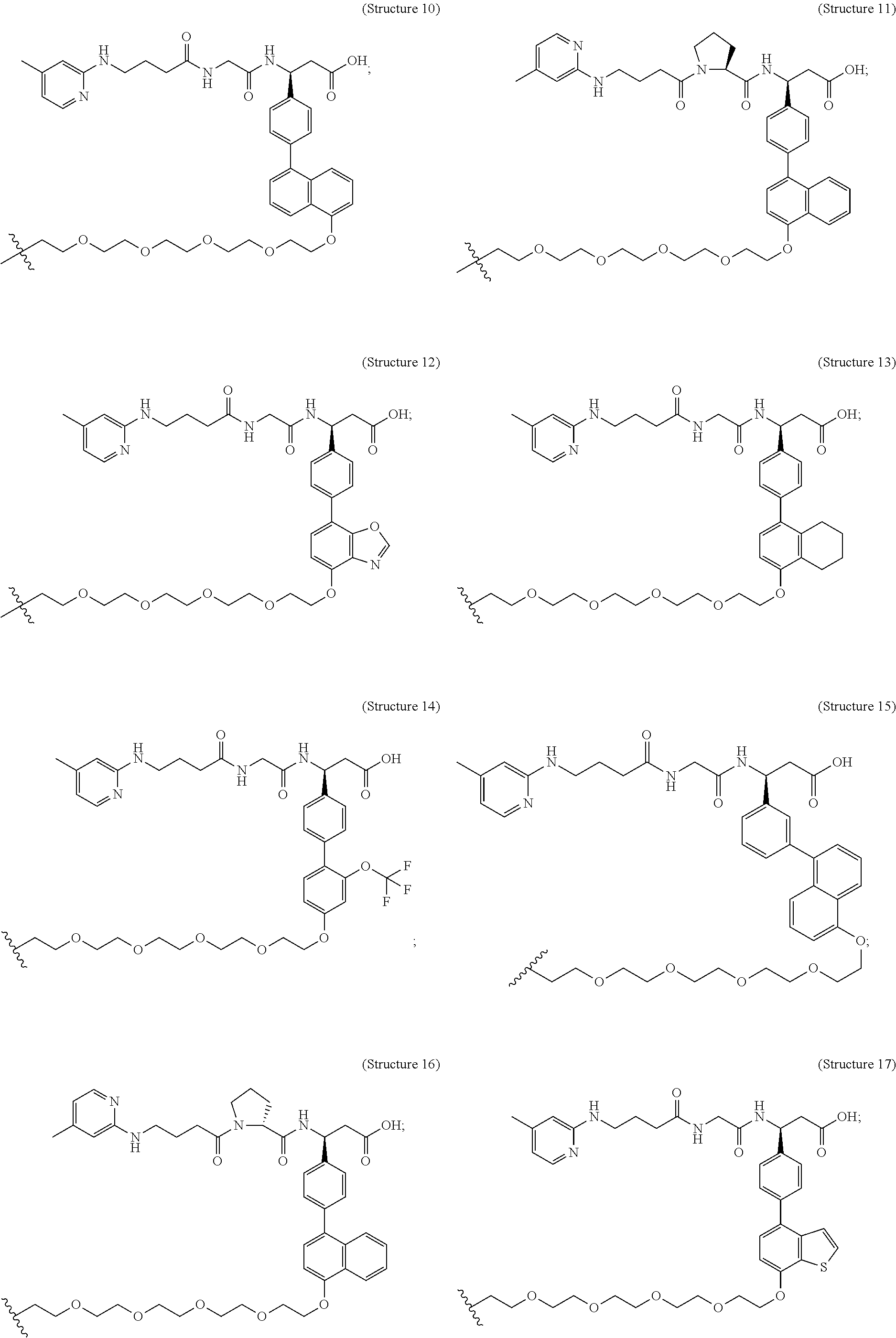 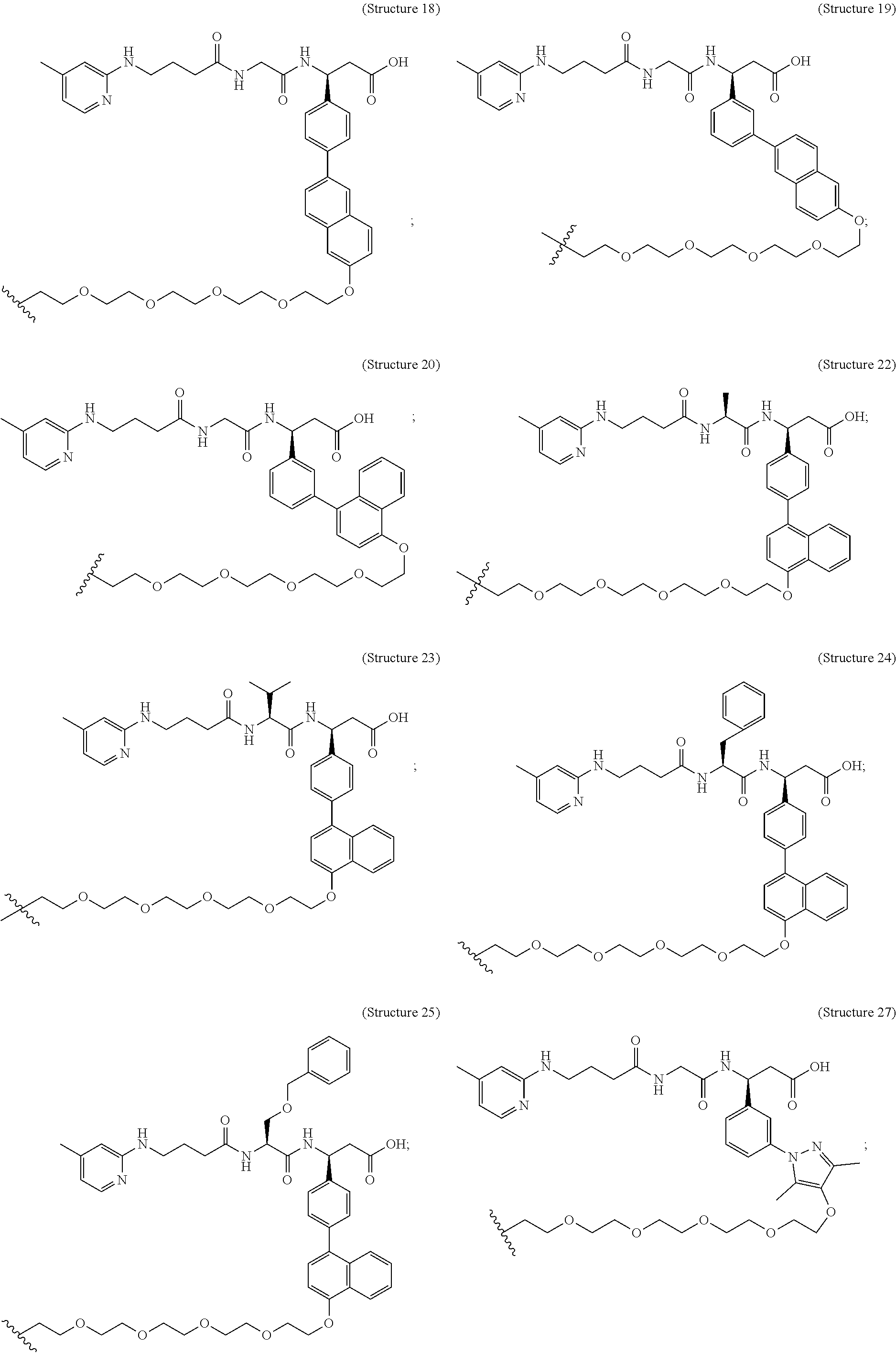 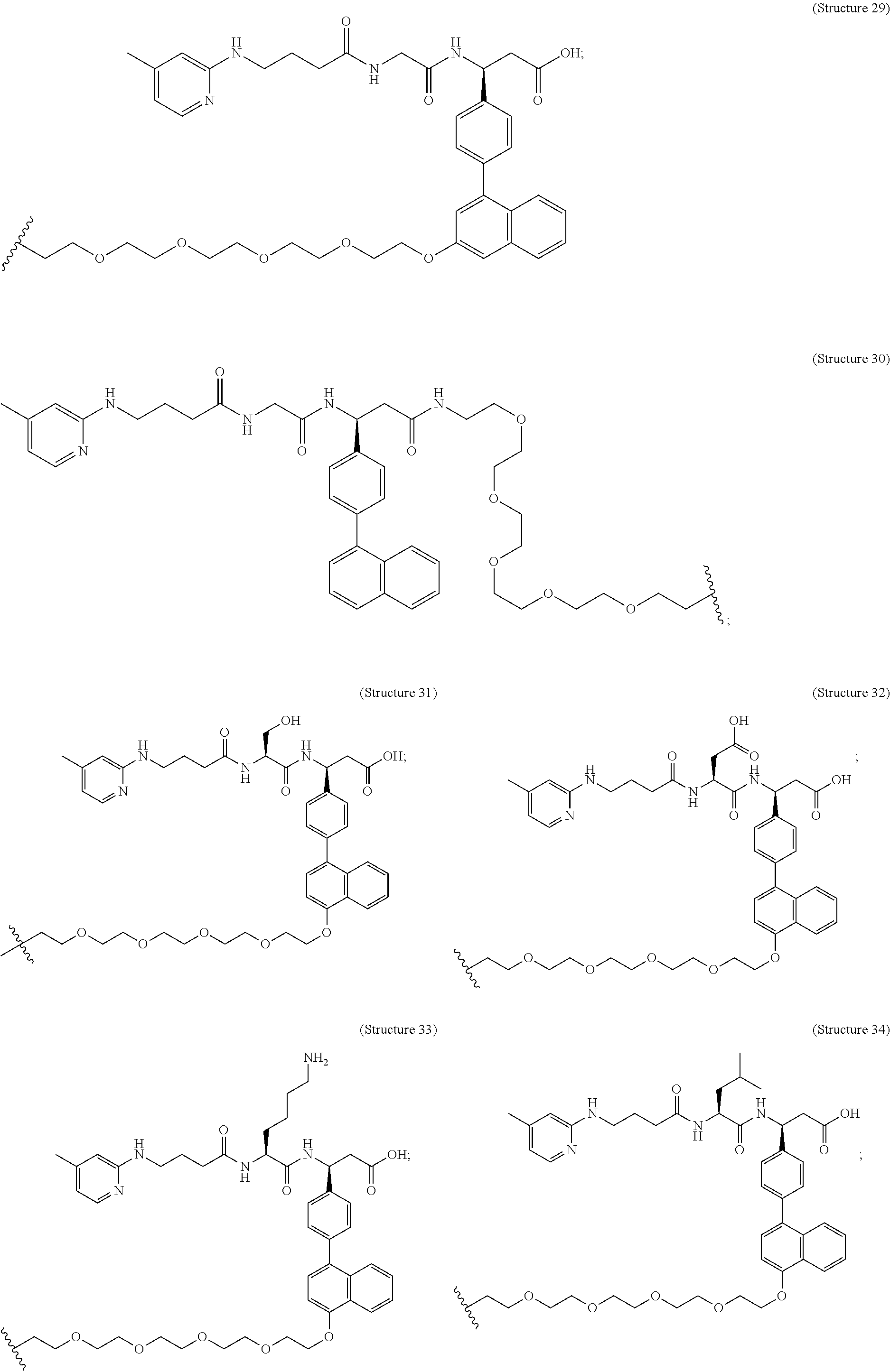 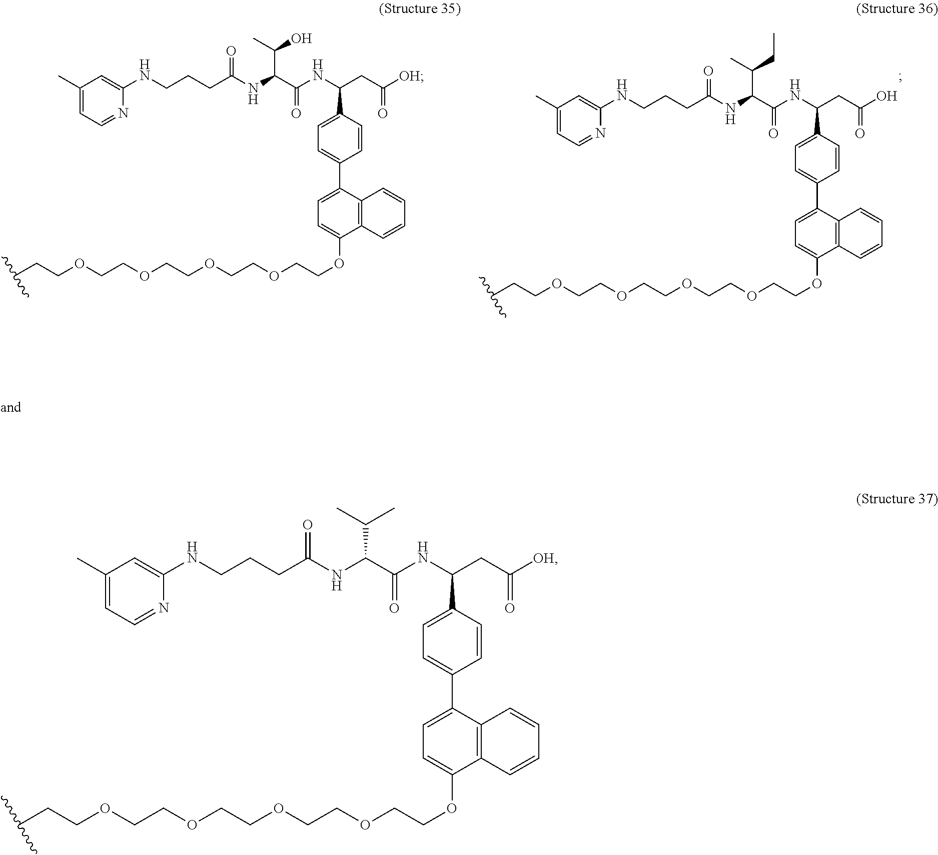 [SMILES: *CCOCCOCCOCCOCCOc1ccc(-c2ccc([C@H](CC(=O)O)NC(=O)[C@@H](NC(=O)CCCNc3cc(C)ccn3)[C@@H](C)O)cc2)c2ccccc12 ; *CCOCCOCCOCCOCCOc1ccc(-c2ccc([C@H](CC(=O)O)NC(=O)[C@@H](NC(=O)CCCNc3cc(C)ccn3)[C@@H](C)CC)cc2)c2ccccc12 ; *CCOCCOCCOCCOCCOc1ccc(-c2ccc([C@H](CC(=O)O)NC(=O)[C@H](NC(=O)CCCNc3cc(C)ccn3)C(C)C)cc2)c2ccccc12]
or a pharmaceutically acceptable salt thereof, wherein [symbol] indicates the point of connection to the RNAi agent.

6. (canceled)

7. The delivery platform of claim 1, wherein the targeting ligand has the formula: 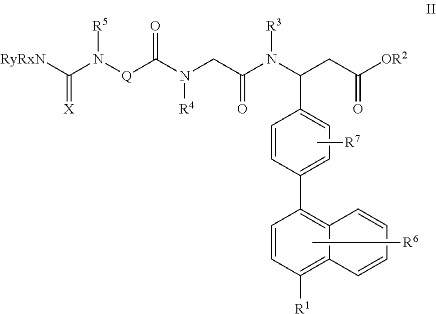
or a pharmaceutically acceptable salt thereof, wherein
R¹ is optionally substituted alkyl, optionally substituted alkoxy, or 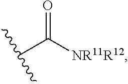
wherein R¹¹ and R¹² are each independently optionally substituted alkyl;
R² is H or optionally substituted alkyl;
R³ is H or optionally substituted alkyl;
R⁴ is H or optionally substituted alkyl;
R⁵ is H or optionally substituted alkyl;
R⁶ is selected from the group consisting of H, optionally substituted alkyl, optionally substituted alkoxy, halo, optionally substituted amino;
Q is optionally substituted aryl or optionally substituted alkylene;
X is O, CR⁸R⁹, NW;
wherein R⁸ is selected from H, optionally substituted alkyl, or R⁸ is taken together with Rx or Ry to form a 4-, 5-, 6-, 7-, 8- or 9-membered ring, and R⁹ is H or optionally substituted alkyl;
Rx and Ry are each independently H, optionally substituted alkyl, or Rx and Ry may be taken together to form a double bond with R¹⁰, wherein R¹⁰ is H, optionally substituted alkyl, or R¹⁰ may be taken together with X and the atoms to which it is attached to form a 4-, 5-, 6-, 7-, 8, or 9-membered ring;
wherein at least one of R¹, R², R⁶, R¹¹, R¹², Rx and Ry comprise a cargo molecule; and
wherein when Q is optionally substituted alkyl and the length of the optionally substituted alkyl chain represented by Q is 3 carbons, then R¹ is

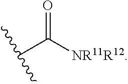

8. The delivery platform of claim 7, wherein the targeting ligand comprises a structure selected from the group consisting of:

| Compound | |
|---|---|
| Number | Formula |
| 40b | 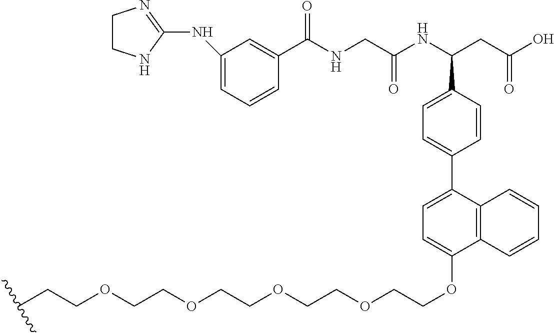 [SMILES: *CCOCCOCCOCCOCCOc1ccc(-c2ccc([C@H](CC(=O)O)NC(=O)CNC(=O)c3cccc(NC4=NCCN4)c3)cc2)c2ccccc12] |
| 41b | 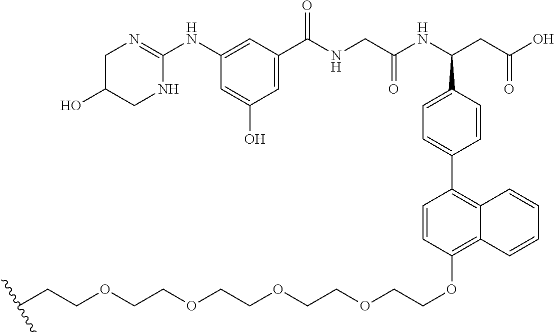 [SMILES: *CCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42b | 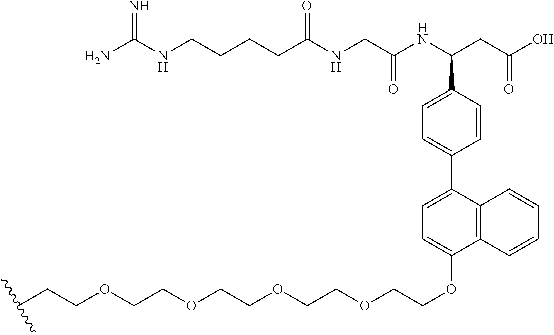 [SMILES: *CCOCCOCCOCCOCCOc1ccc(-c2ccc([C@H](CC(=O)O)NC(=O)CNC(=O)CCCCNC(=N)N)cc2)c2ccccc12] |
| 43b | 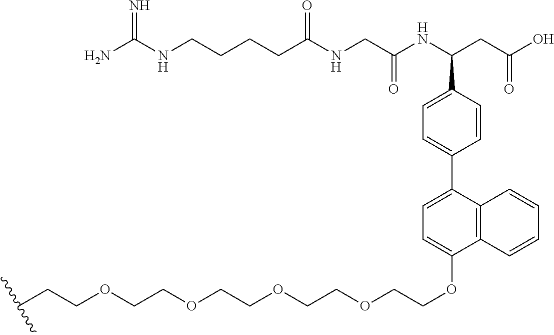 [SMILES: *CCOCCOCCOCCOCCOc1ccc(-c2ccc([C@H](CC(=O)O)NC(=O)CNC(=O)CCCCNC(=N)N)cc2)c2ccccc12] |
| 44b | 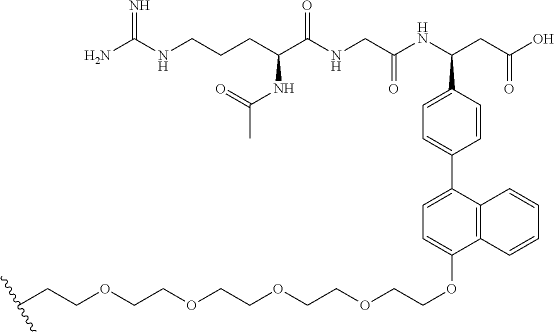 [SMILES: *CCOCCOCCOCCOCCOc1ccc(-c2ccc([C@H](CC(=O)O)NC(=O)CNC(=O)[C@H](CCCNC(=N)N)NC(C)=O)cc2)c2ccccc12] |
| 45b | 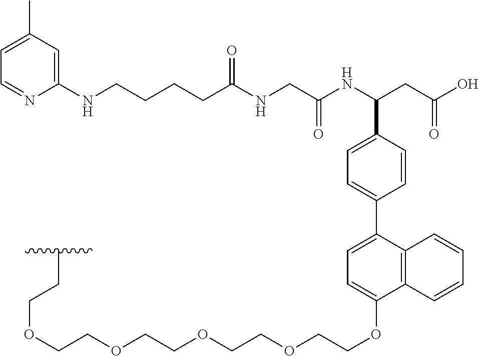 [SMILES: *CCOCCOCCOCCOCCOc1ccc(-c2ccc([C@H](CC(=O)O)NC(=O)CNC(=O)CCCCNc3cc(C)ccn3)cc2)c2ccccc12] |
| 46b | 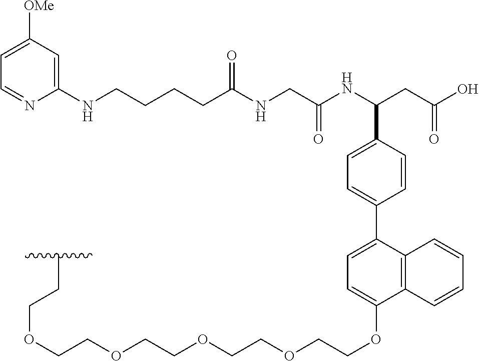 [SMILES: *CCOCCOCCOCCOCCOc1ccc(-c2ccc([C@H](CC(=O)O)NC(=O)CNC(=O)CCCCNc3cc(OC)ccn3)cc2)c2ccccc12] |
| 47b | 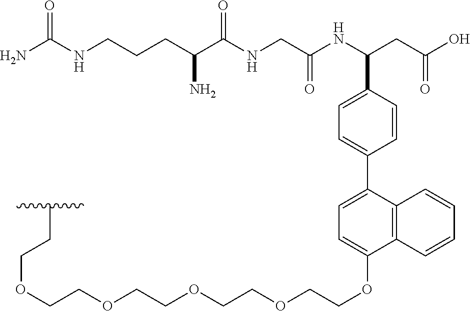 [SMILES: *CCOCCOCCOCCOCCOc1ccc(-c2ccc([C@H](CC(=O)O)NC(=O)CNC(=O)[C@@H](N)CCCNC(N)=O)cc2)c2ccccc12] |
| 48b | 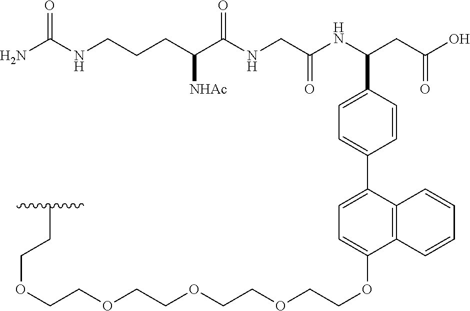 [SMILES: *CCOCCOCCOCCOCCOc1ccc(-c2ccc([C@H](CC(=O)O)NC(=O)CNC(=O)[C@H](CCCNC(N)=O)NC(C)=O)cc2)c2ccccc12] |
| 49b | 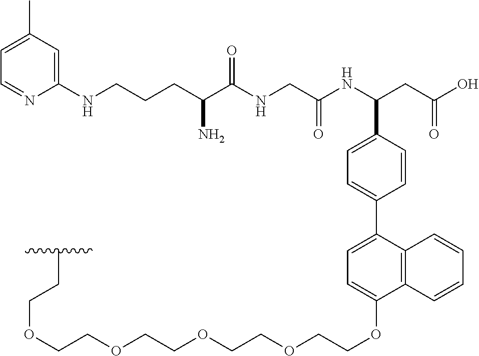 [SMILES: *CCOCCOCCOCCOCCOc1ccc(-c2ccc([C@H](CC(=O)O)NC(=O)CNC(=O)[C@@H](N)CCCNc3cc(C)ccn3)cc2)c2ccccc12] |
| 50b | 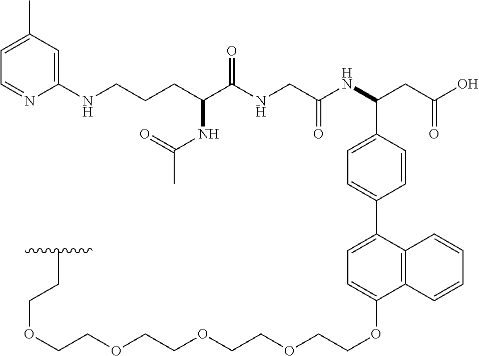 [SMILES: *CCOCCOCCOCCOCCOc1ccc(-c2ccc([C@H](CC(=O)O)NC(=O)CNC(=O)[C@H](CCCNc3cc(C)ccn3)NC(C)=O)cc2)c2ccccc12] |
| 51b | 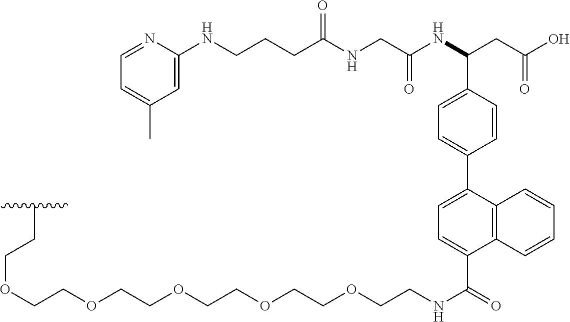 [SMILES: *CCOCCOCCOCCOCCOCCNC(=O)c1ccc(-c2ccc([C@H](CC(=O)O)NC(=O)CNC(=O)CCCNc3cc(C)ccn3)cc2)c2ccccc12] |
| 52b | 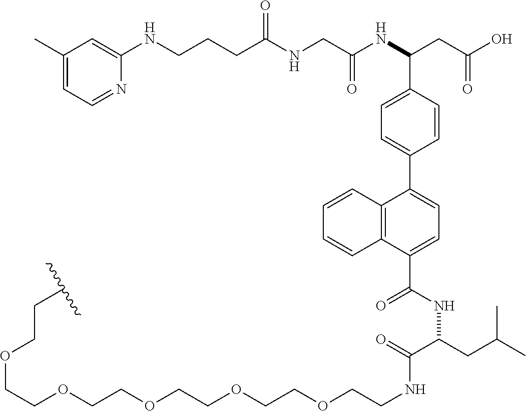 |
| 53b | 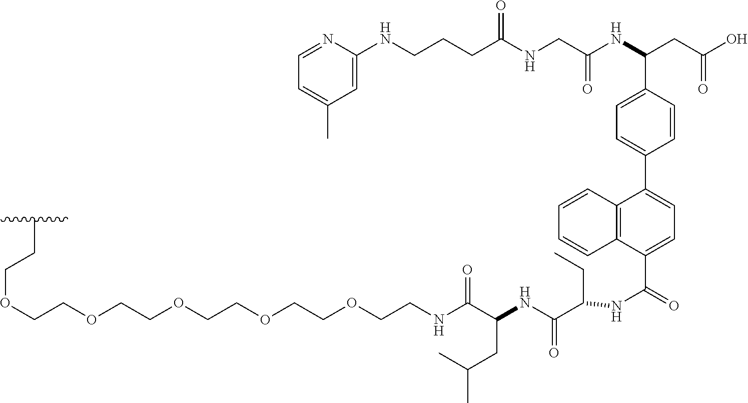 |
| 54b | 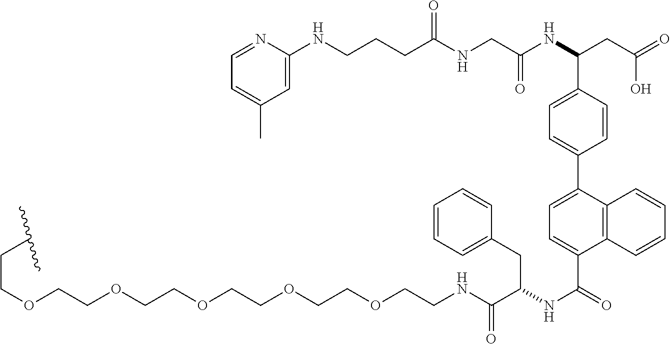 |
| 55b | 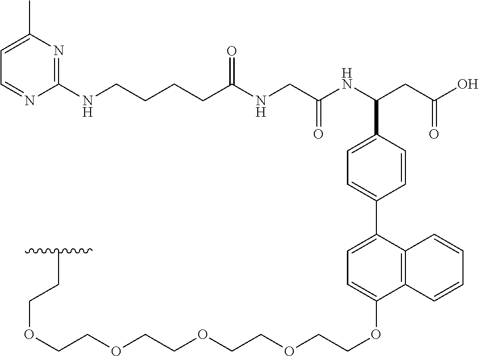 [SMILES: *CCOCCOCCOCCOCCOc1ccc(-c2ccc([C@H](CC(=O)O)NC(=O)CNC(=O)CCCCNc3nccc(C)n3)cc2)c2ccccc12] |
| 56b | 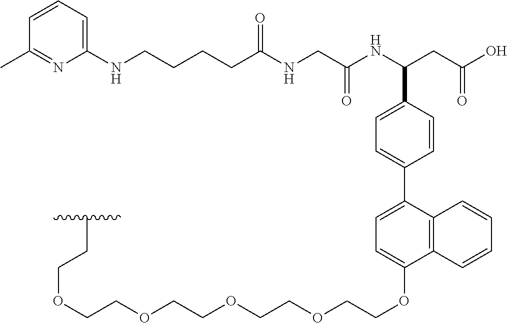 [SMILES: *CCOCCOCCOCCOCCOc1ccc(-c2ccc([C@H](CC(=O)O)NC(=O)CNC(=O)CCCCNc3cccc(C)n3)cc2)c2ccccc12] |
| 57b | 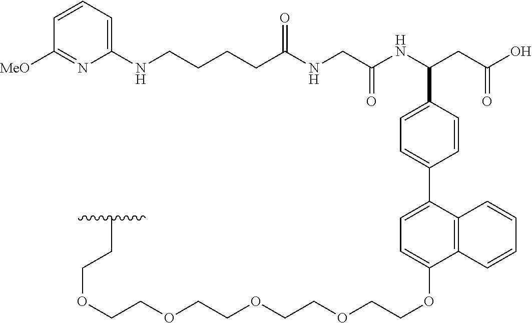 [SMILES: *CCOCCOCCOCCOCCOc1ccc(-c2ccc([C@H](CC(=O)O)NC(=O)CNC(=O)CCCCNc3cccc(OC)n3)cc2)c2ccccc12] |
| 58b | 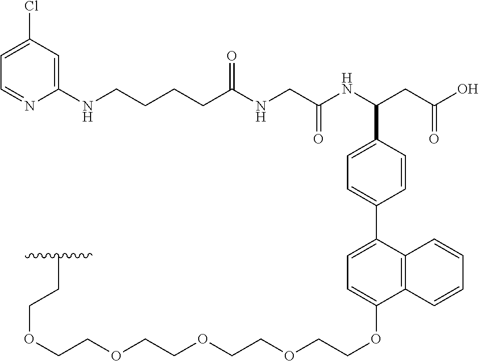 [SMILES: *CCOCCOCCOCCOCCOc1ccc(-c2ccc([C@H](CC(=O)O)NC(=O)CNC(=O)CCCCNc3cc(Cl)ccn3)cc2)c2ccccc12] |
| 59b | 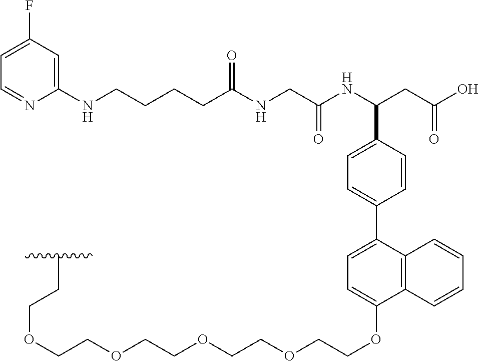 [SMILES: *CCOCCOCCOCCOCCOc1ccc(-c2ccc([C@H](CC(=O)O)NC(=O)CNC(=O)CCCCNc3cc(F)ccn3)cc2)c2ccccc12] |
| 60b | 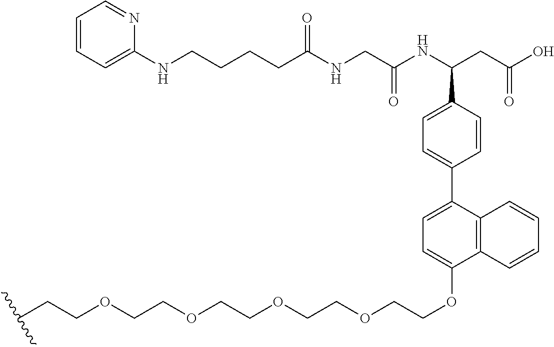 [SMILES: *CCOCCOCCOCCOCCOc1ccc(-c2ccc([C@H](CC(=O)O)NC(=O)CNC(=O)CCCCNc3ccccn3)cc2)c2ccccc12] |

or a pharmaceutically acceptable salt thereof, and wherein [symbol] indicates the point of connection to a cargo molecule.

9. (canceled)

10. The delivery platform of claim 2, wherein the PK/PD modulator comprises at least one polyethylene glycol (PEG) unit.

11. (canceled)

12. The delivery platform of claim 10, wherein the PK/PD modulator is selected from the group consisting of: 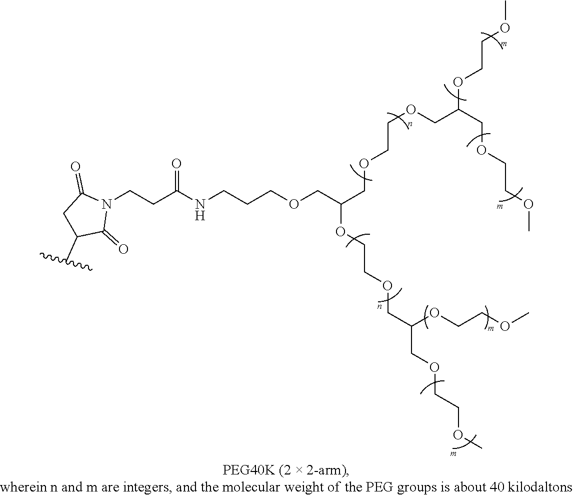 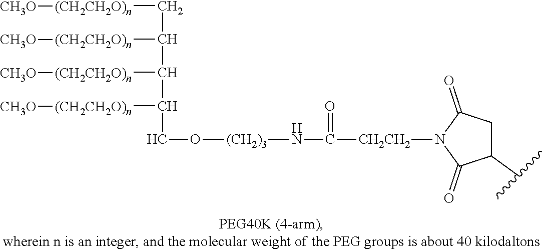 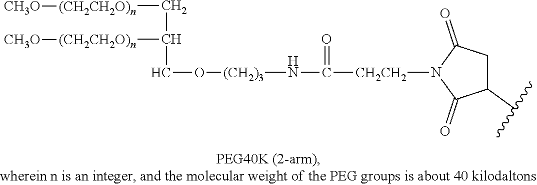 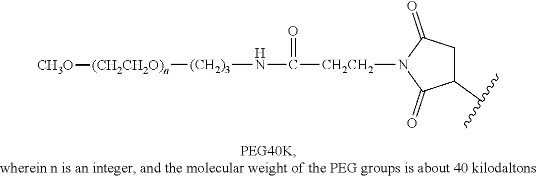 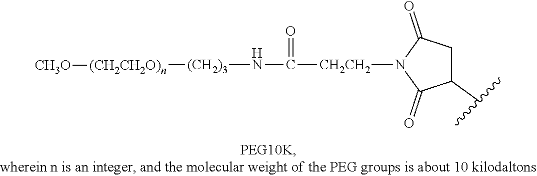 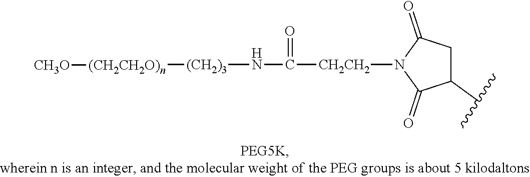 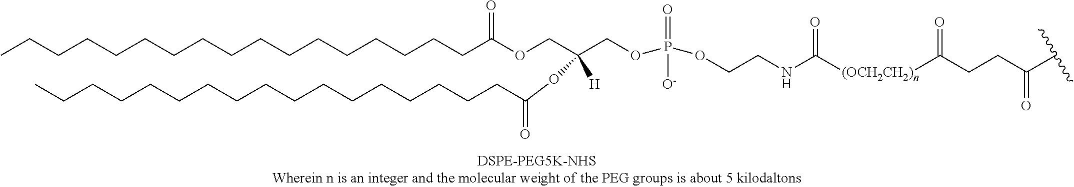 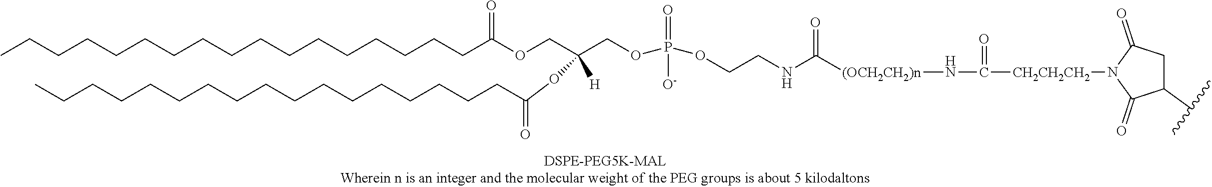 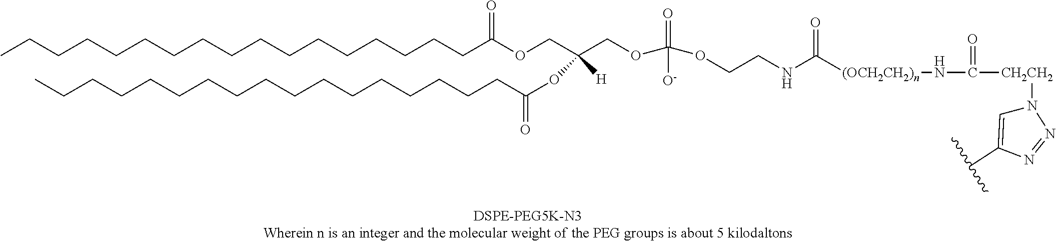 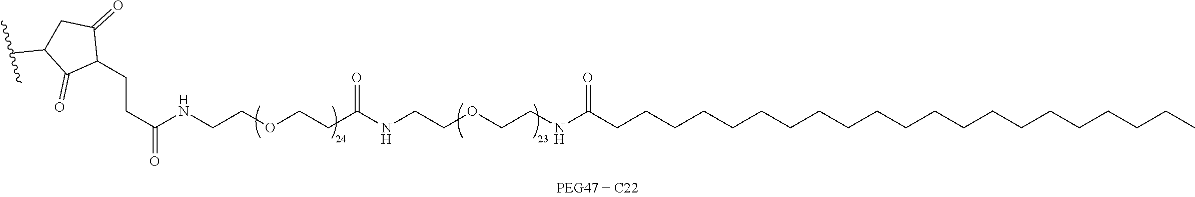 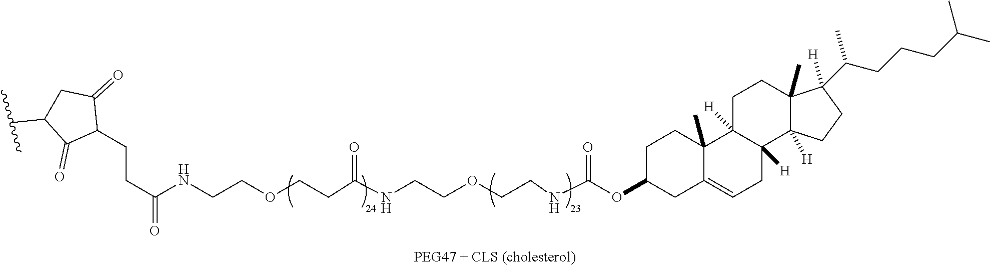 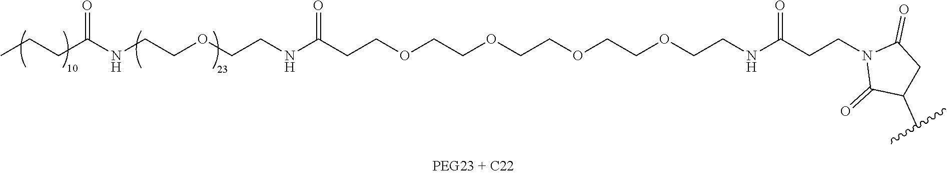 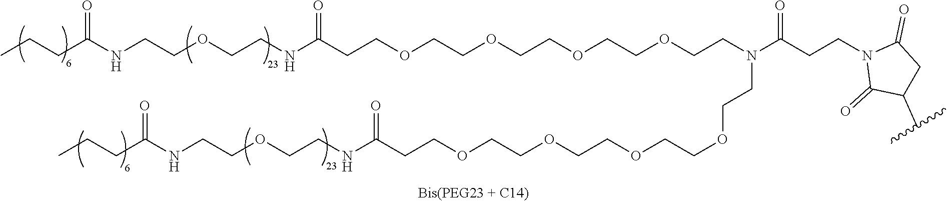 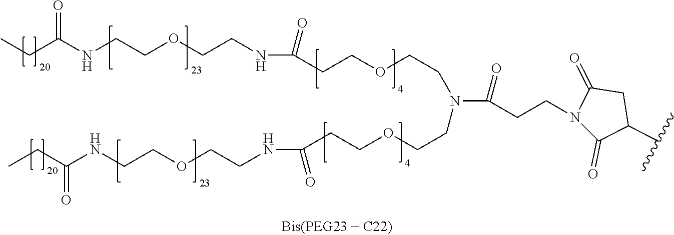 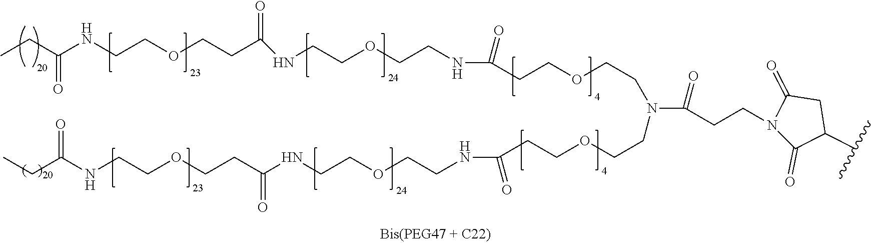 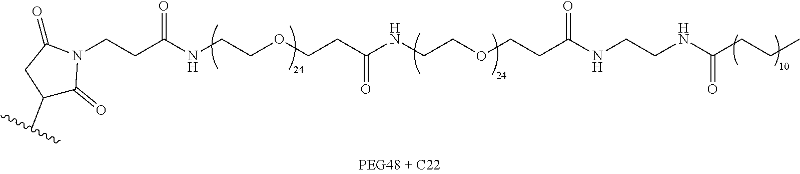 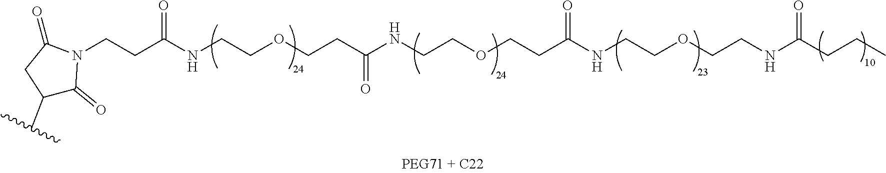 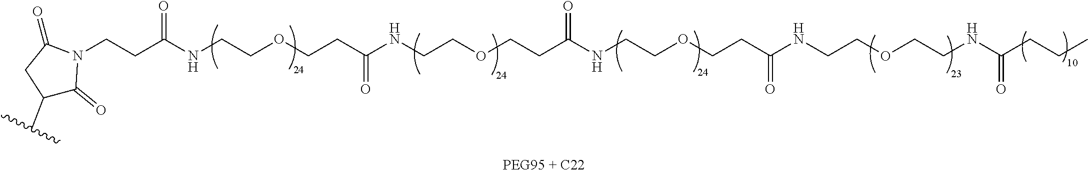 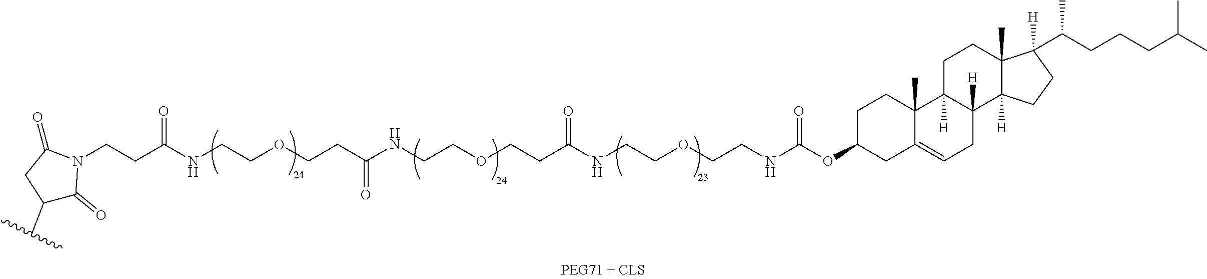 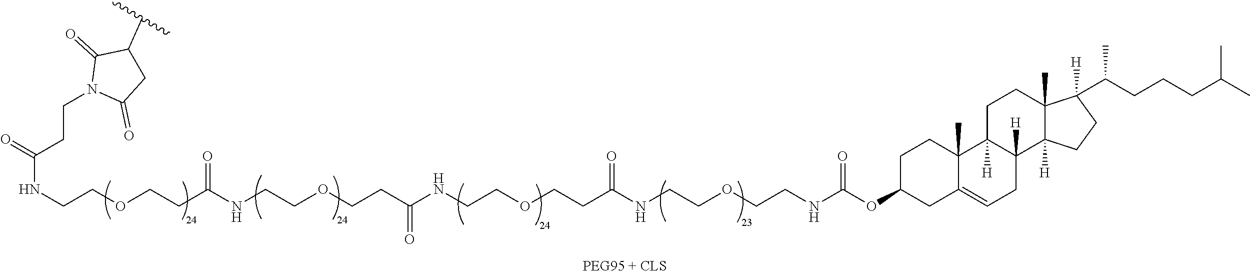 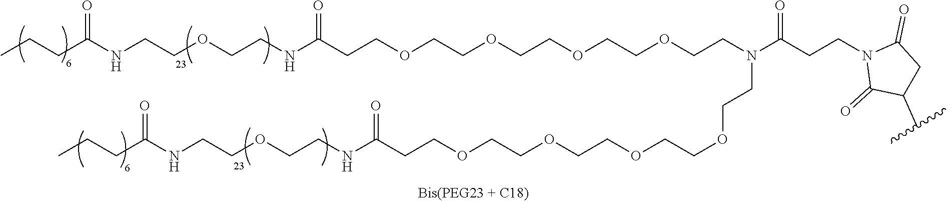 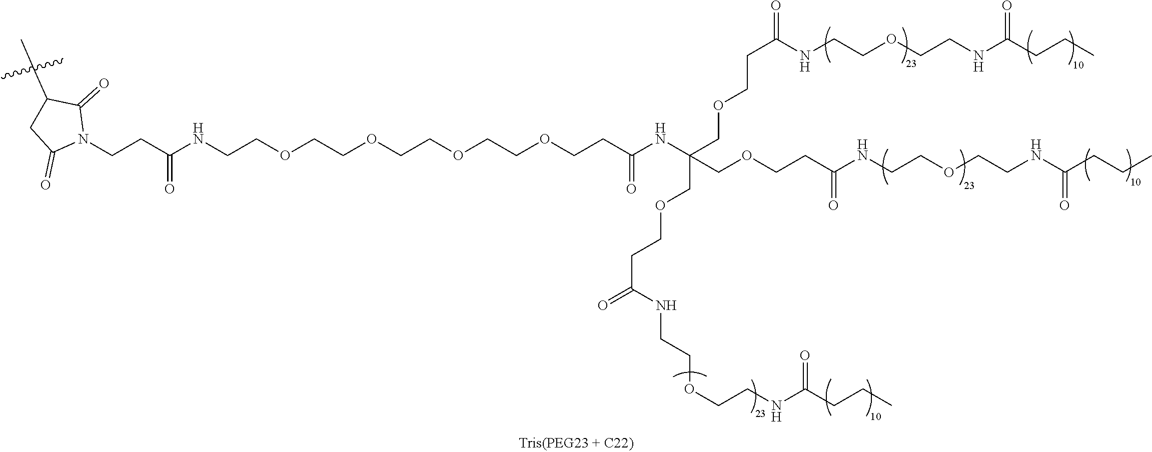 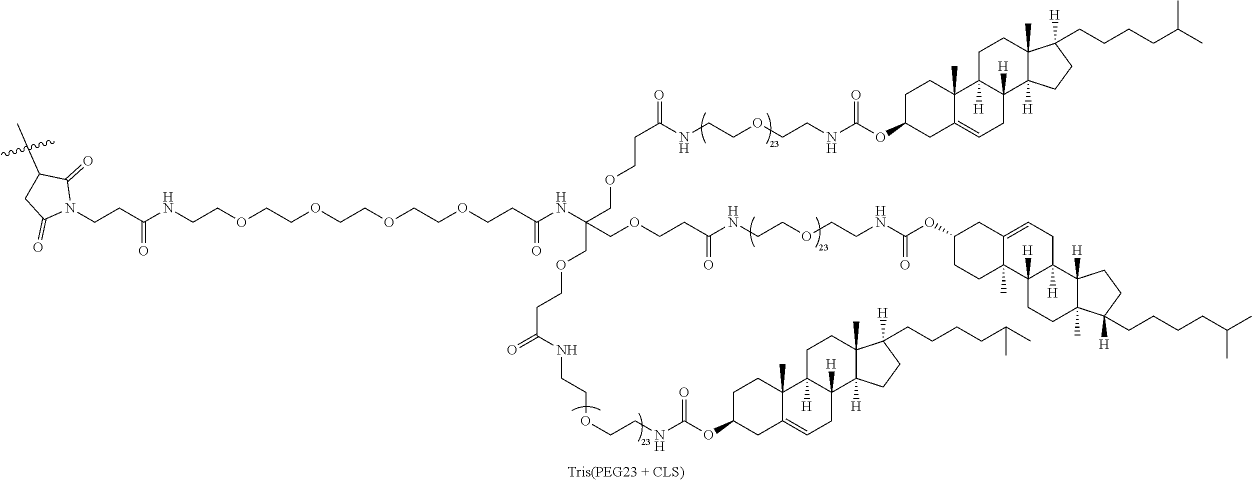 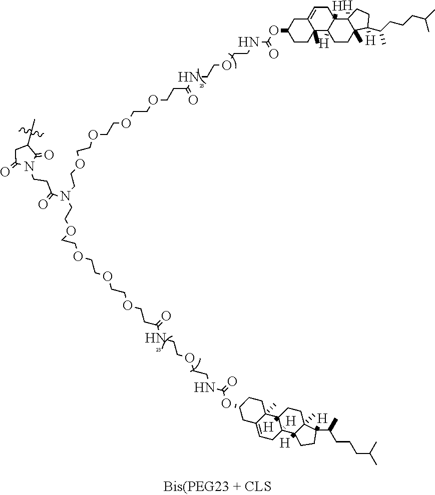 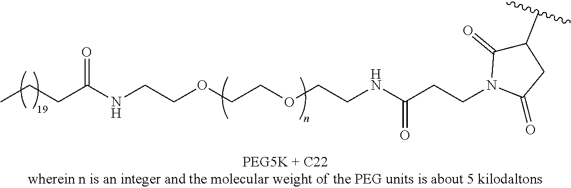 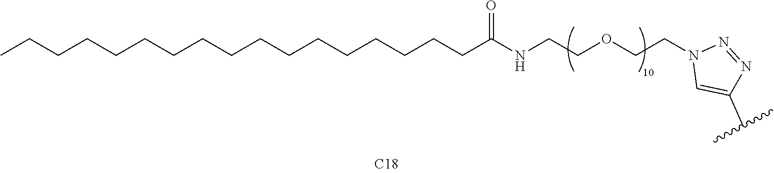 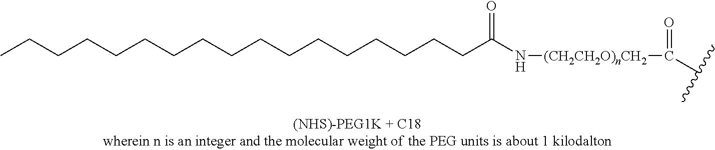 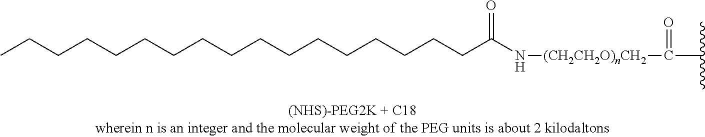 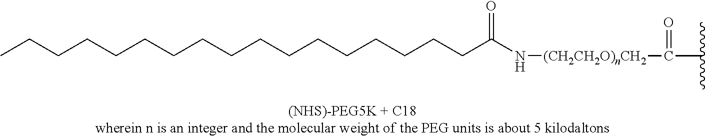 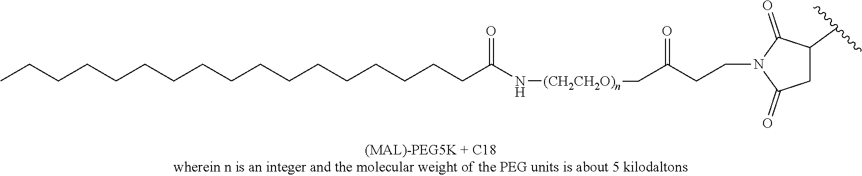 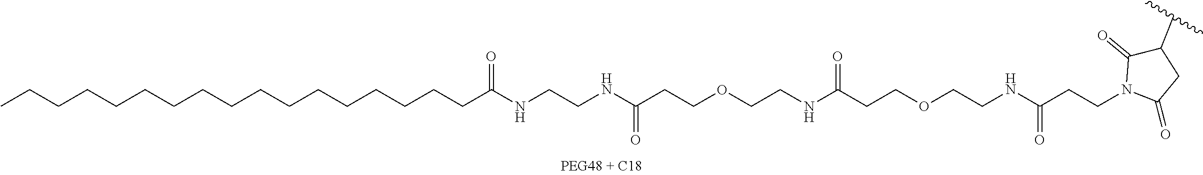 [SMILES: *C1CC(=O)N(CCC(=O)NCCOCCC(=O)NCCOCCC(=O)NCCNC(=O)CCCCCCCCCCCCCCCCC)C1=O]
wherein [symbol] indicates the point of attachment to the RNAi agent.

13. The delivery platform of claim 2, wherein the PK/PD modulator has the formula: 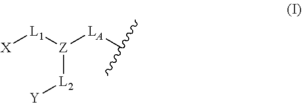
or a pharmaceutically acceptable salt thereof, wherein
L_{A} is a bond or a bivalent moiety connecting Z to the RNAi agent;
Z is CH, phenyl, or N;
L₁ and L₂ are each independently linkers comprising at least about 5 PEG units;
X and Y are each independently lipids comprising from about 10 to about 50 carbon atoms; and [symbol]
indicates a point of connection to the RNAi agent.

14-18. (canceled)

19. The delivery platform of claim 13, wherein at least one of X and Y is an unsaturated lipid.

20-23. (canceled)

24. The delivery platform of claim 13, wherein at least one of X and Y is cholesteryl.

25. The delivery platform of claim 13, wherein at least one of X and Y is selected from the group consisting of:

| Name | Structure |
|---|---|
| Lipid 1 | 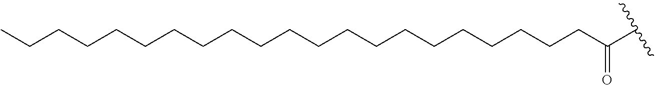 [SMILES: *C(=O)CCCCCCCCCCCCCCCCCCCCC] |
| Lipid 2 | 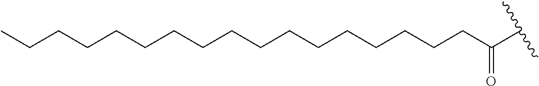 [SMILES: *C(=O)CCCCCCCCCCCCCCCCC] |
| Lipid 3 | 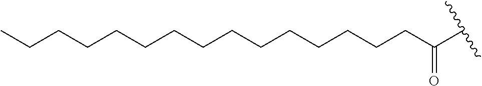 [SMILES: *C(=O)CCCCCCCCCCCCCCC] |
| Lipid 4 | 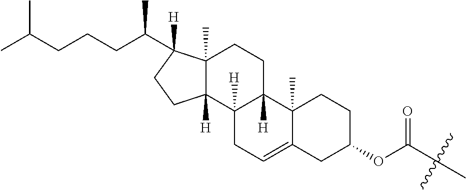 |
| Lipid 5 | 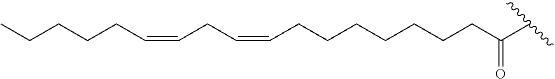 [SMILES: *C(=O)CCCCCCC/C=C\C/C=C\CCCCC] |
| Lipid 6 | 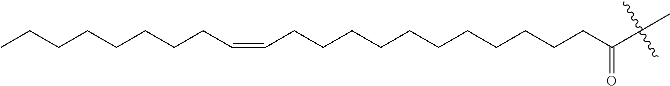 [SMILES: *C(=O)CCCCCCCCCCC/C=C\CCCCCCCC] |
| Lipid 7 | 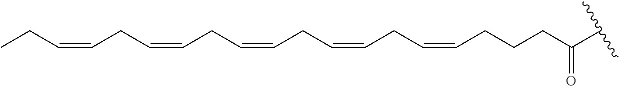 [SMILES: *C(=O)CCC/C=C\C/C=C\C/C=C\C/C=C\C/C=C\CC] |
| Lipid 8 | 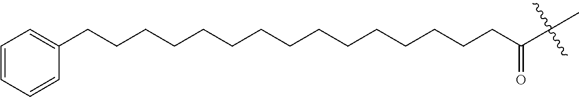 [SMILES: *C(=O)CCCCCCCCCCCCCCCc1ccccc1] |
| Lipid 9 | 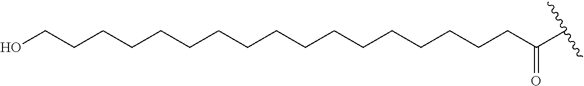 [SMILES: *C(=O)CCCCCCCCCCCCCCCCCO] |
| Lipid 10 | 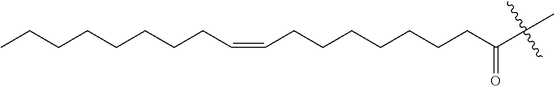 [SMILES: *C(=O)CCCCCCC/C=C\CCCCCCCC] |
| Lipid 11 | 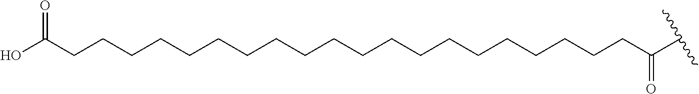 [SMILES: *C(=O)CCCCCCCCCCCCCCCCCCCCC(=O)O] |
| Lipid 12 | 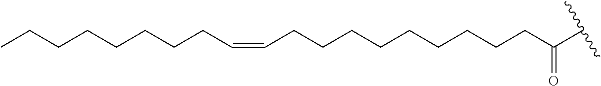 [SMILES: *C(=O)CCCCCCCCC/C=C\CCCCCCCC] |
| Lipid 14 | 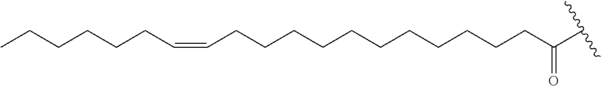 [SMILES: *C(=O)CCCCCCCCCCC/C=C\CCCCCC] |
| Lipid 15 | 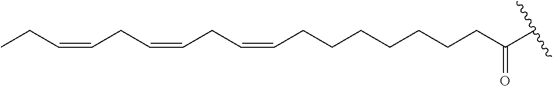 [SMILES: *C(=O)CCCCCCC/C=C\C/C=C\C/C=C\CC] |
| Lipid 16 | 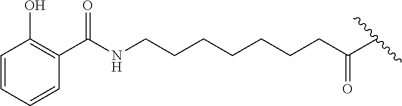 [SMILES: *C(=O)CCCCCCCNC(=O)c1ccccc1O] |
| Lipid 17 | 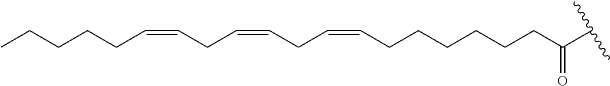 [SMILES: *C(=O)CCCCCC/C=C\C/C=C\C/C=C\CCCCC] |
| Lipid 18 | 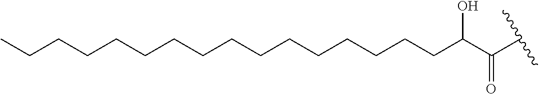 [SMILES: *C(=O)C(O)CCCCCCCCCCCCCCCC] |
| Lipid 19 | 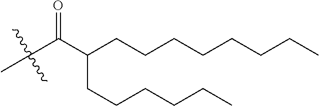 [SMILES: *C(=O)C(CCCCCC)CCCCCCCC] |
| Lipid 20 | 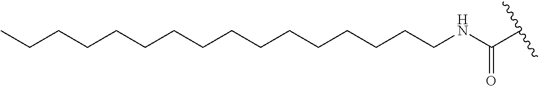 [SMILES: *C(=O)NCCCCCCCCCCCCCCCC] |
| Lipid 21 | 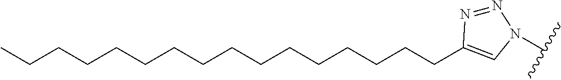 [SMILES: *n1cc(CCCCCCCCCCCCCCCC)nn1] |
| Lipid 22 | 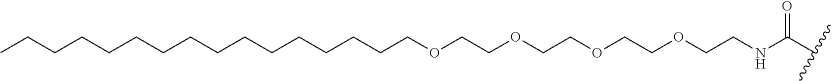 [SMILES: *C(=O)NCCOCCOCCOCCOCCCCCCCCCCCCCCCC] |
| Lipid 23 | 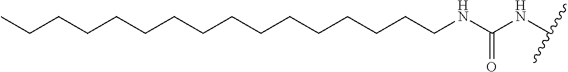 [SMILES: *NC(=O)NCCCCCCCCCCCCCCCC] |
| Lipid 24 | 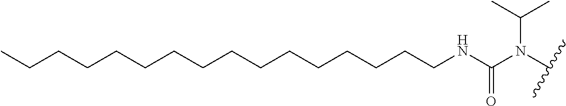 [SMILES: *N(C(=O)NCCCCCCCCCCCCCCCC)C(C)C] |

wherein [symbol] indicates the point of connection to the remainder of the compound.

26. The delivery platform of claim 13, wherein both X and Y are each independently selected from the group consisting of:

| Name | Structure |
|---|---|
| Lipid 1 | 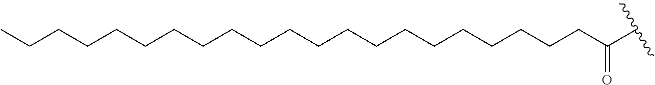 [SMILES: *C(=O)CCCCCCCCCCCCCCCCCCCCC] |
| Lipid 2 | 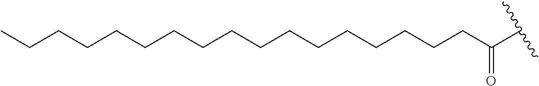 [SMILES: *C(=O)CCCCCCCCCCCCCCCCC] |
| Lipid 3 | 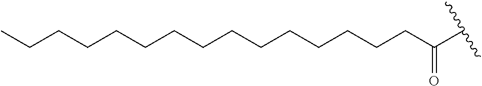 [SMILES: *C(=O)CCCCCCCCCCCCCCC] |
| Lipid 4 | 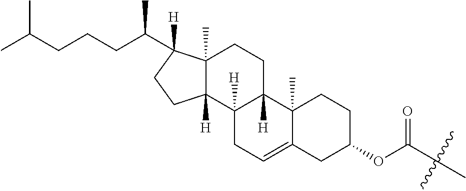 |
| Lipid 5 | 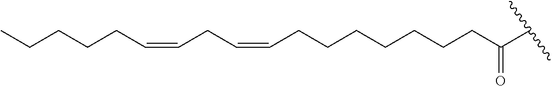 [SMILES: *C(=O)CCCCCCC/C=C\C/C=C\CCCCC] |
| Lipid 6 | 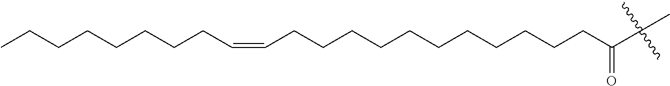 [SMILES: *C(=O)CCCCCCCCCCC/C=C\CCCCCCCC] |
| Lipid 7 | 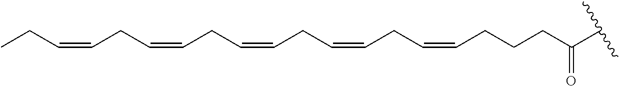 [SMILES: *C(=O)CCC/C=C\C/C=C\C/C=C\C/C=C\C/C=C\CC] |
| Lipid 8 | 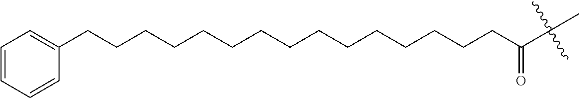 [SMILES: *C(=O)CCCCCCCCCCCCCCCc1ccccc1] |
| Lipid 9 | 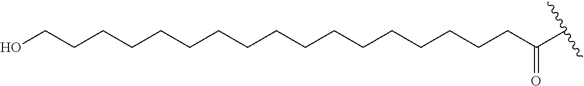 [SMILES: *C(=O)CCCCCCCCCCCCCCCCCO] |
| Lipid 10 | 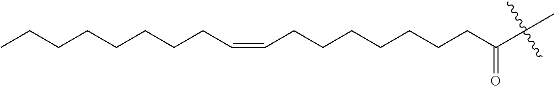 [SMILES: *C(=O)CCCCCCC/C=C\CCCCCCCC] |
| Lipid 11 | 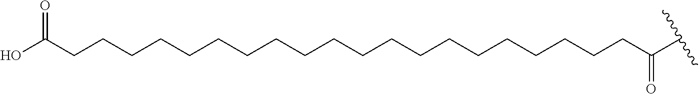 [SMILES: *C(=O)CCCCCCCCCCCCCCCCCCCCC(=O)O] |
| Lipid 12 | 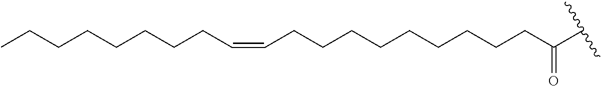 [SMILES: *C(=O)CCCCCCCCC/C=C\CCCCCCCC] |
| Lipid 14 | 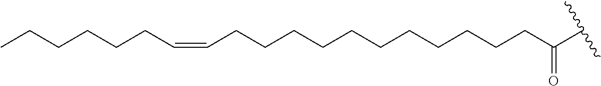 [SMILES: *C(=O)CCCCCCCCCCC/C=C\CCCCCC] |
| Lipid 15 | 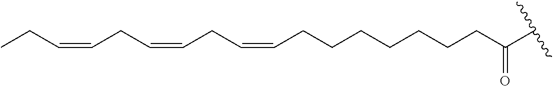 [SMILES: *C(=O)CCCCCCC/C=C\C/C=C\C/C=C\CC] |
| Lipid 16 | 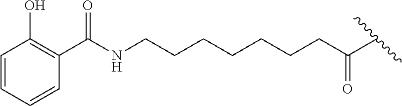 [SMILES: *C(=O)CCCCCCCNC(=O)c1ccccc1O] |
| Lipid 17 | 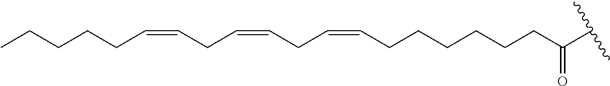 [SMILES: *C(=O)CCCCCC/C=C\C/C=C\C/C=C\CCCCC] |
| Lipid 18 | 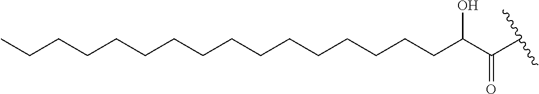 [SMILES: *C(=O)C(O)CCCCCCCCCCCCCCCC] |
| Lipid 19 | 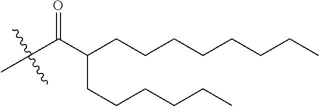 [SMILES: *C(=O)C(CCCCCC)CCCCCCCC] |
| Lipid 20 | 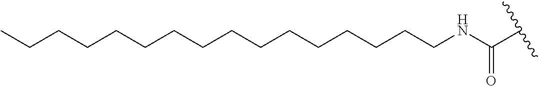 [SMILES: *C(=O)NCCCCCCCCCCCCCCCC] |
| Lipid 21 | 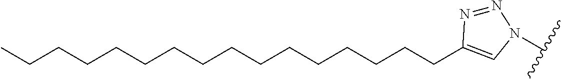 [SMILES: *n1cc(CCCCCCCCCCCCCCCC)nn1] |
| Lipid 22 | 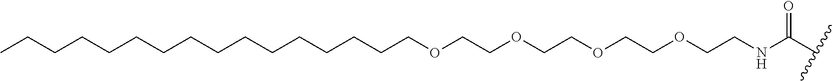 [SMILES: *C(=O)NCCOCCOCCOCCOCCCCCCCCCCCCCCCC] |
| Lipid 23 | 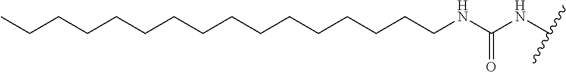 [SMILES: *NC(=O)NCCCCCCCCCCCCCCCC] |
| Lipid 24 | 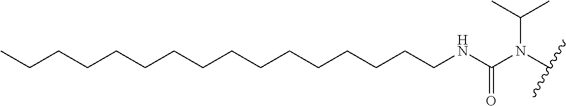 [SMILES: *N(C(=O)NCCCCCCCCCCCCCCCC)C(C)C] |

wherein [symbol] indicates the point of connection to L₁ or L₂.

27. The delivery platform of claim 2, wherein the PK/PD modulator is selected from the group consisting of: 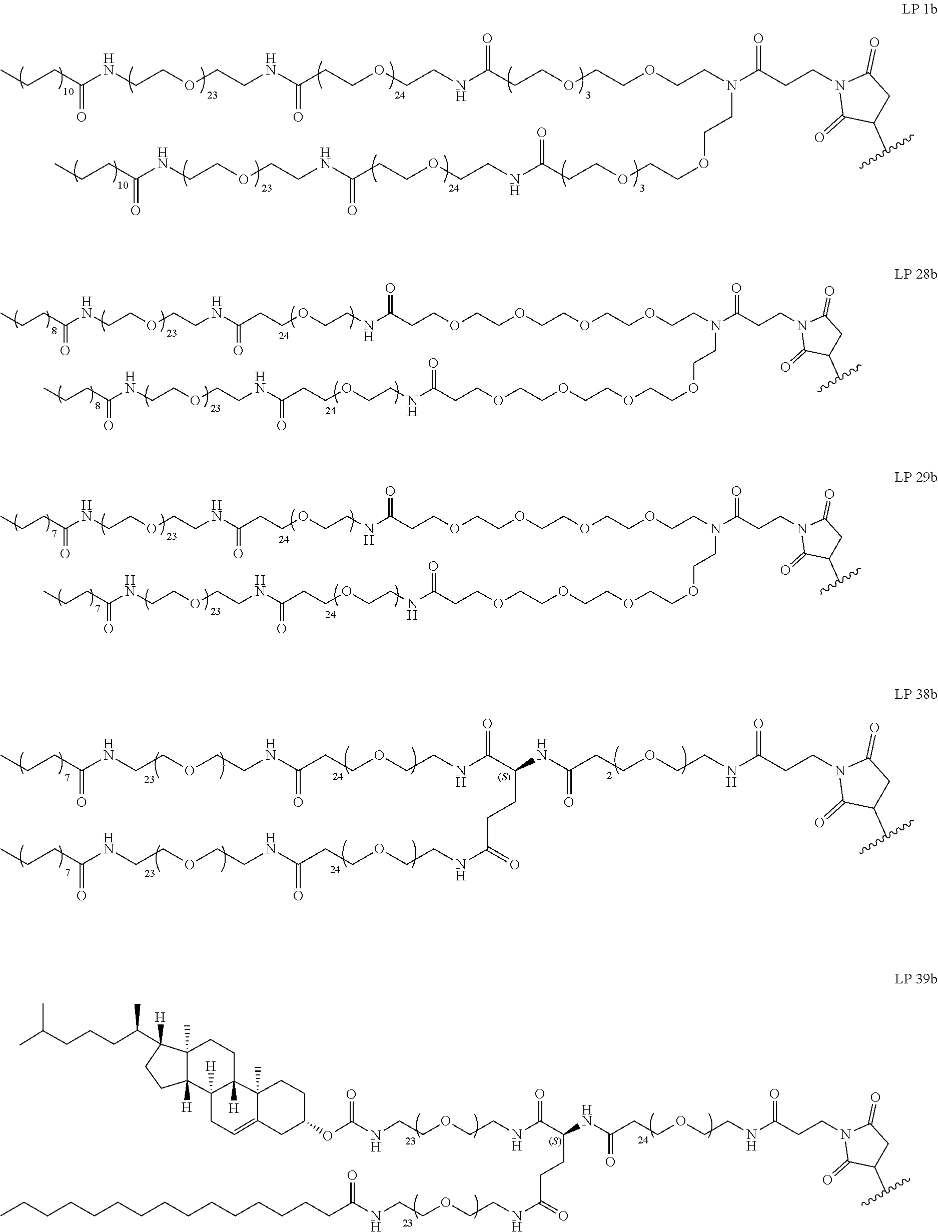 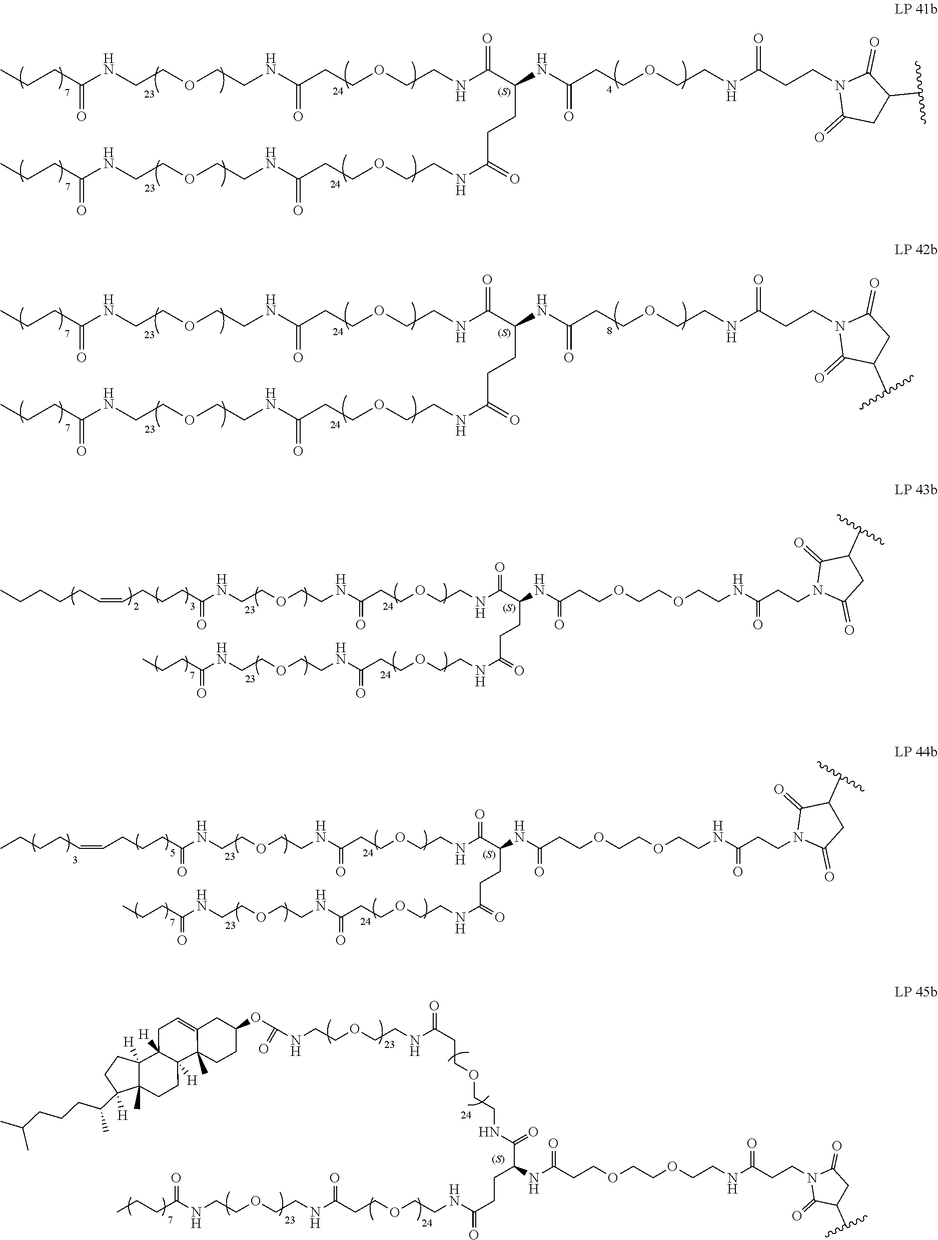 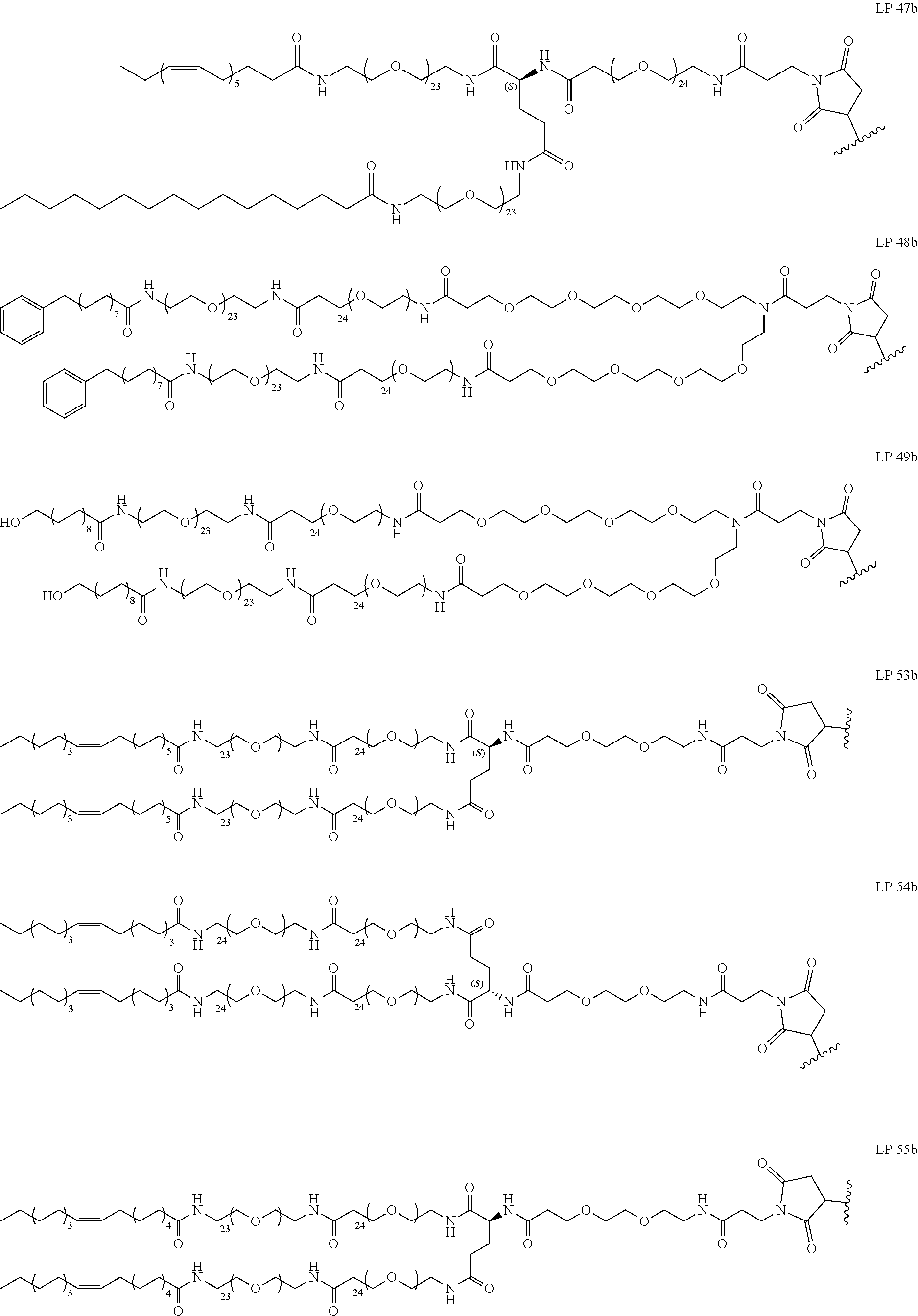 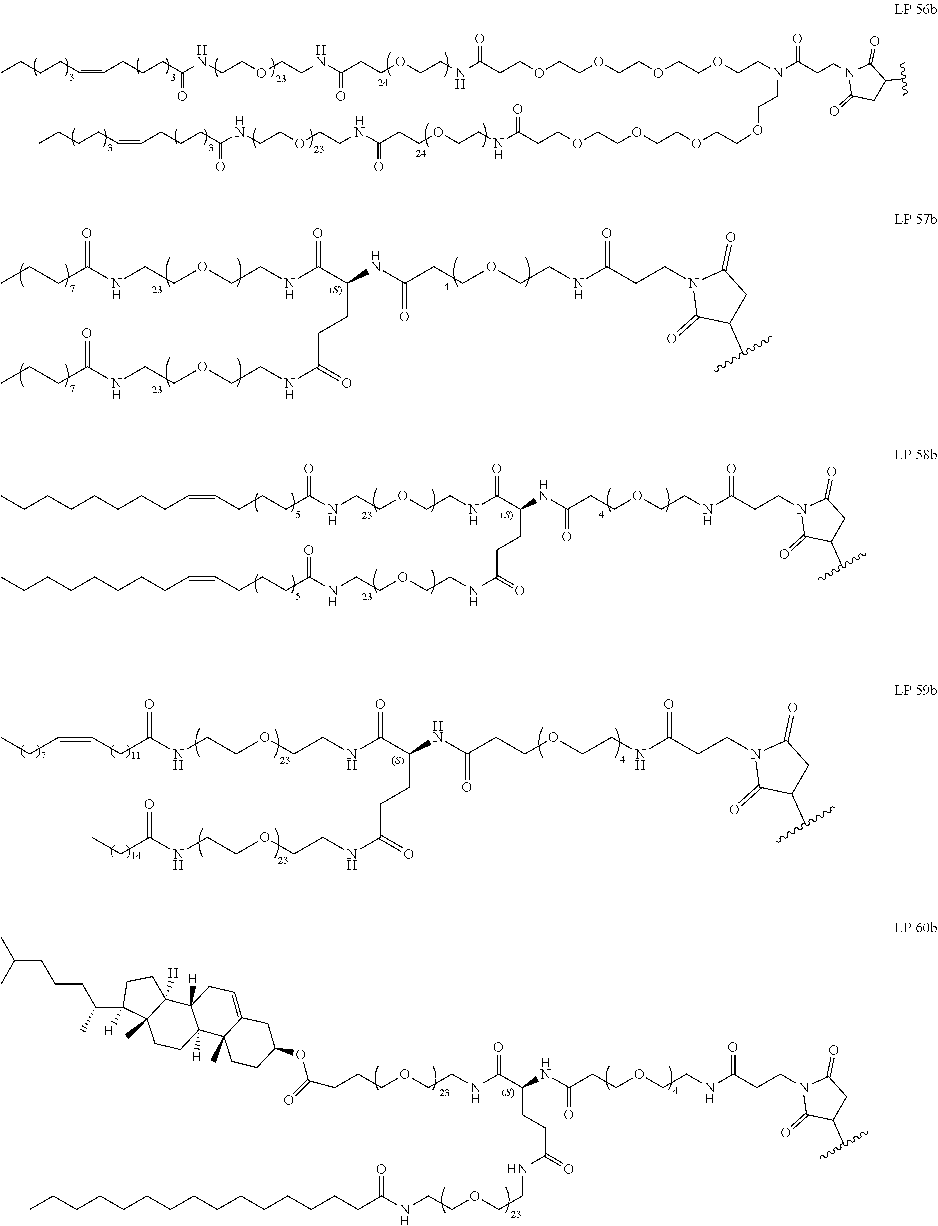 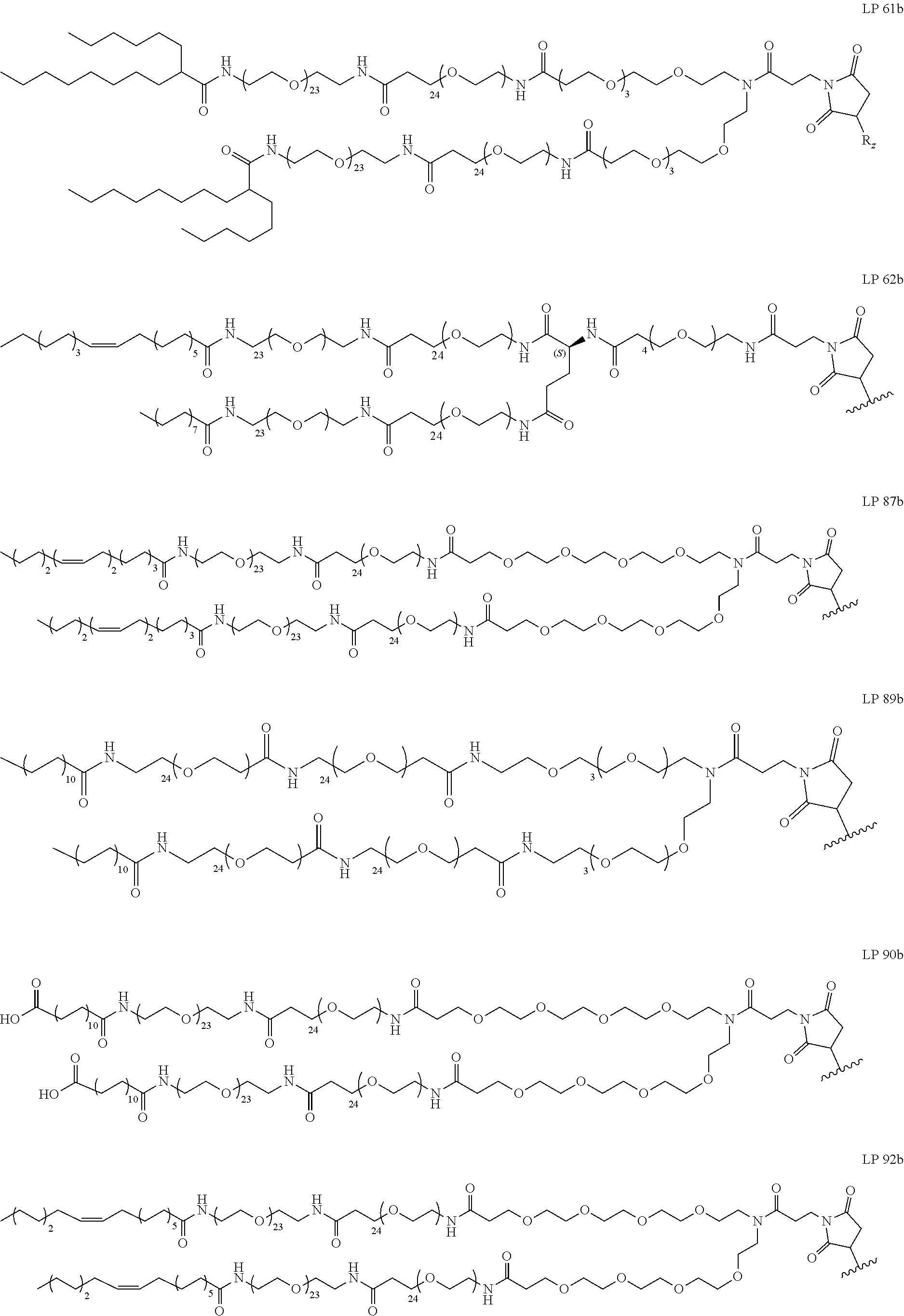 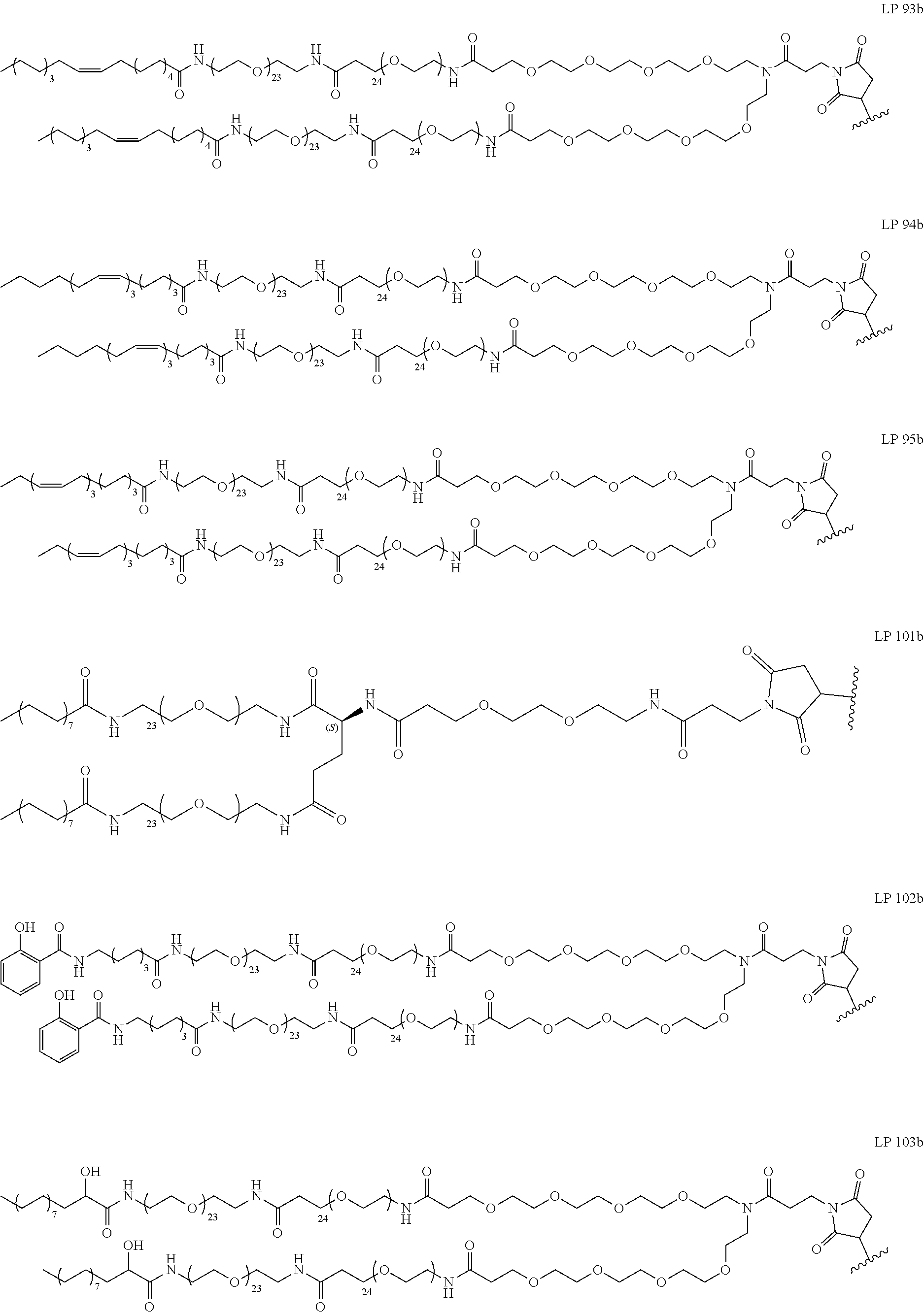 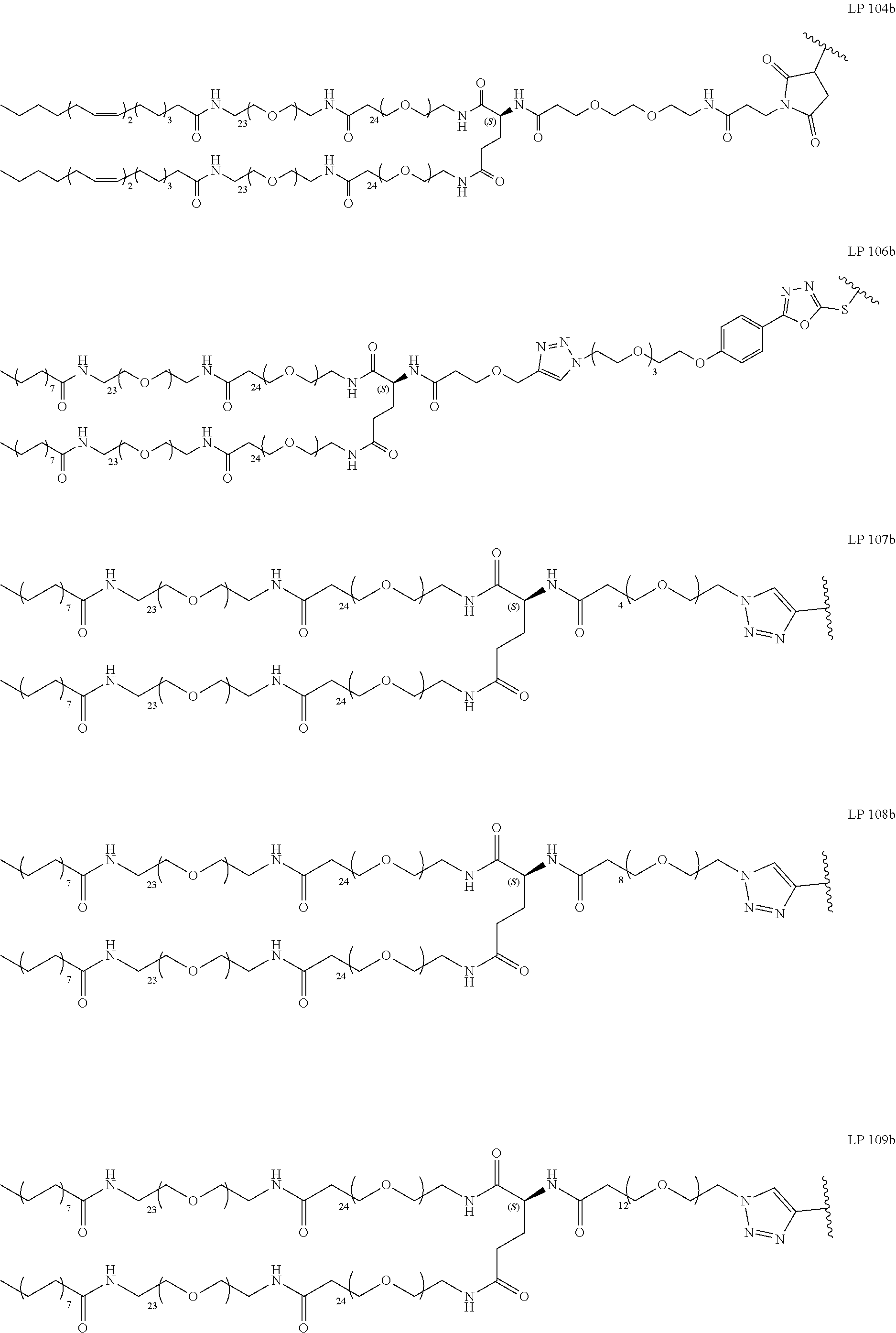 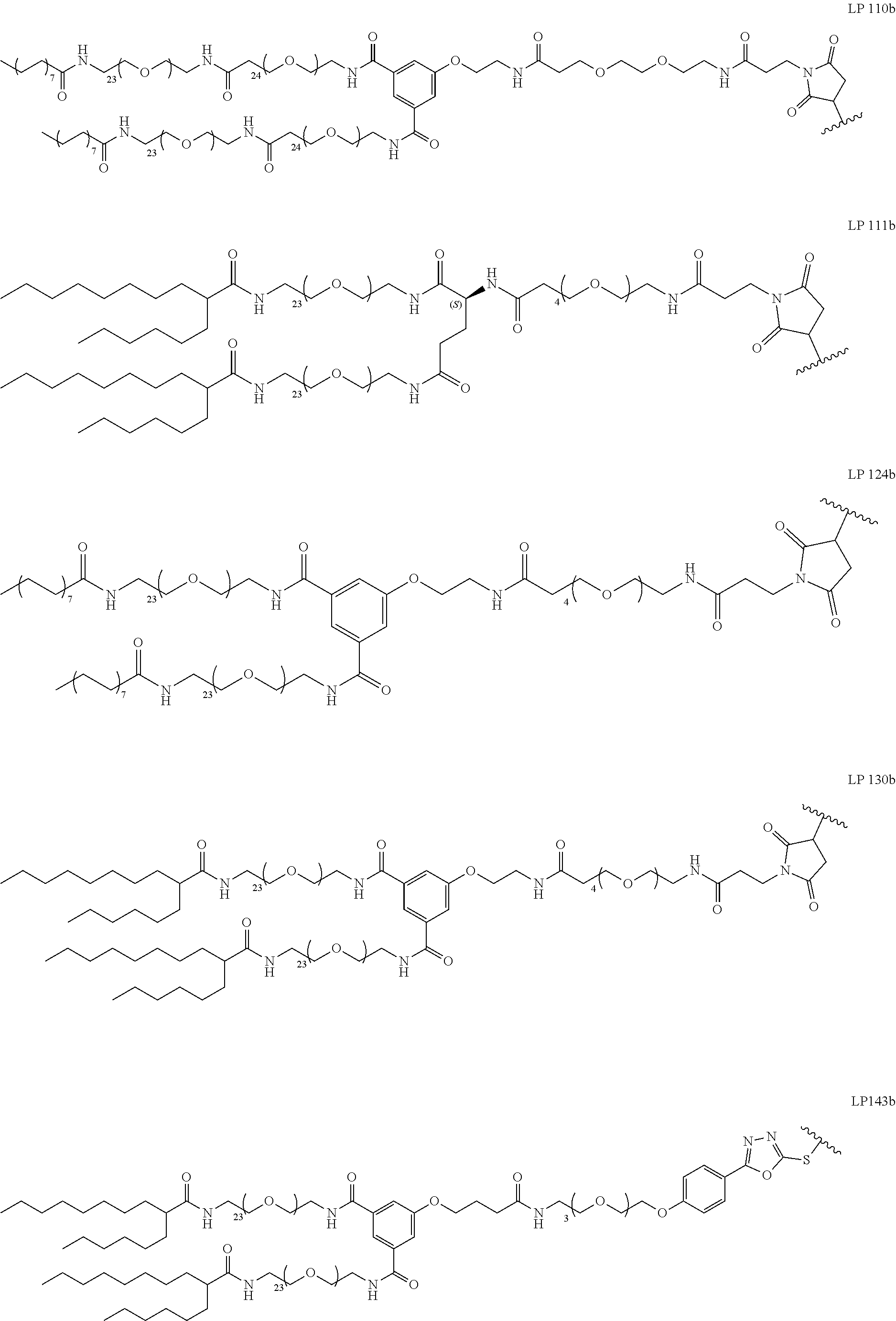 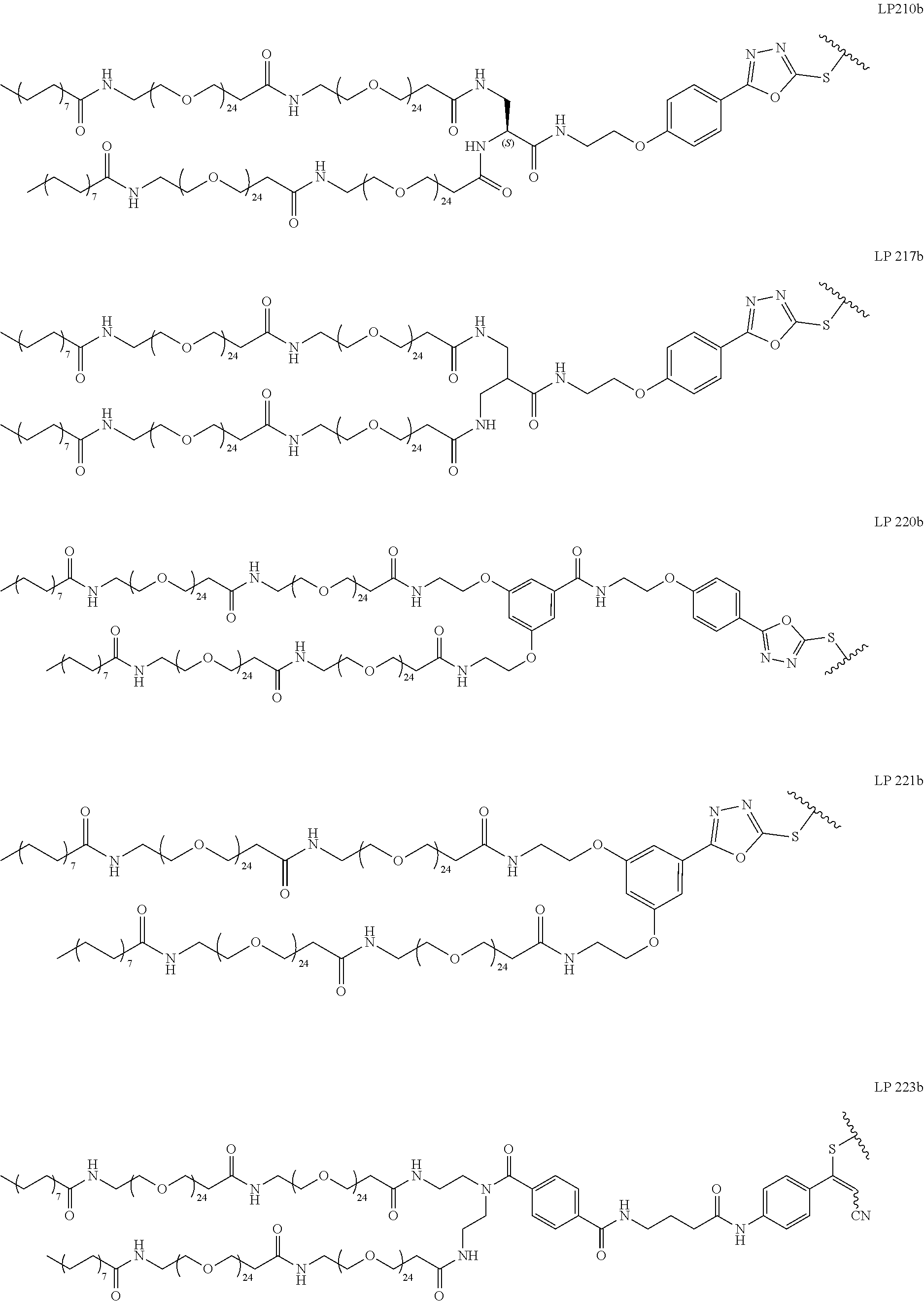 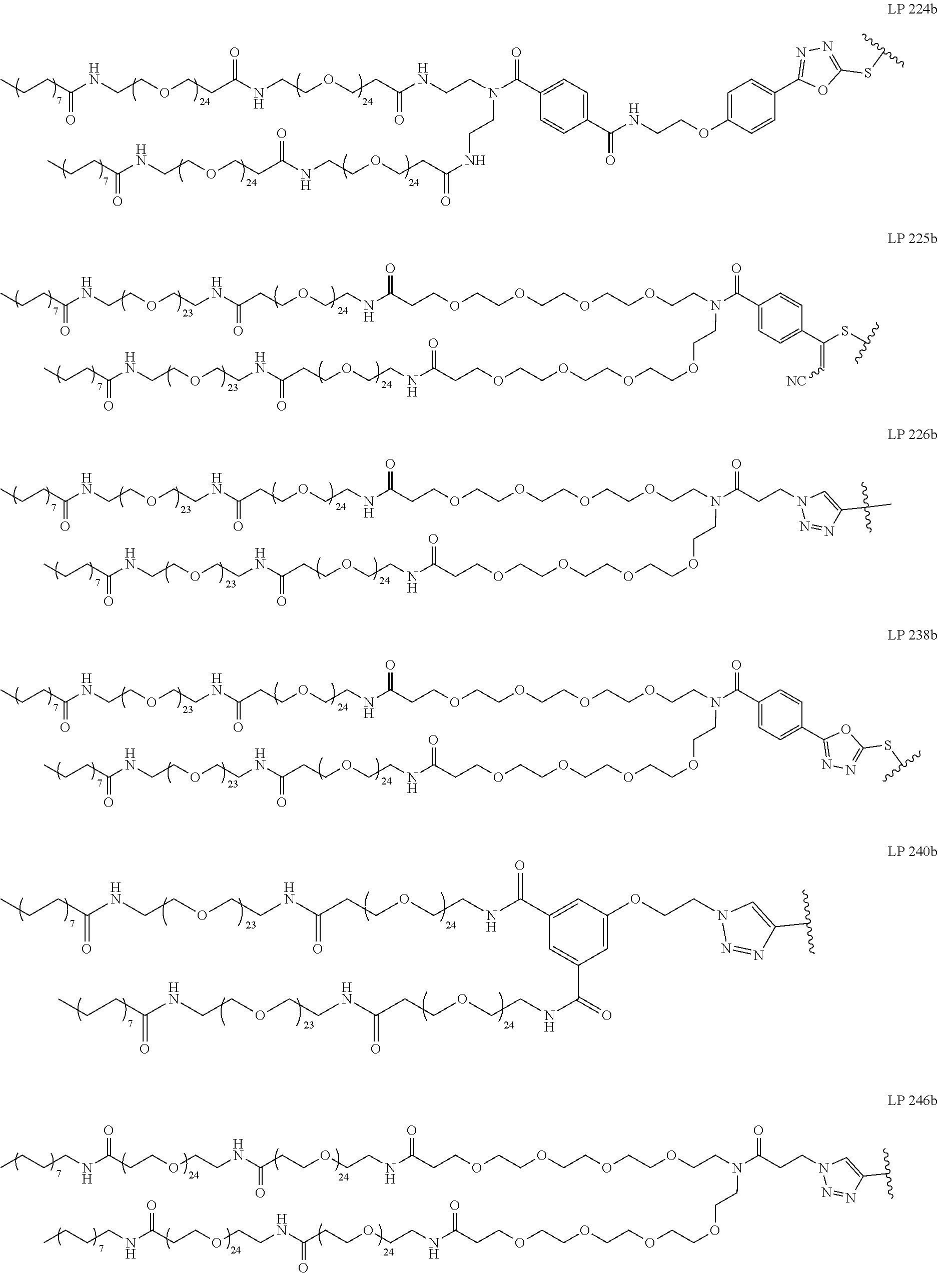 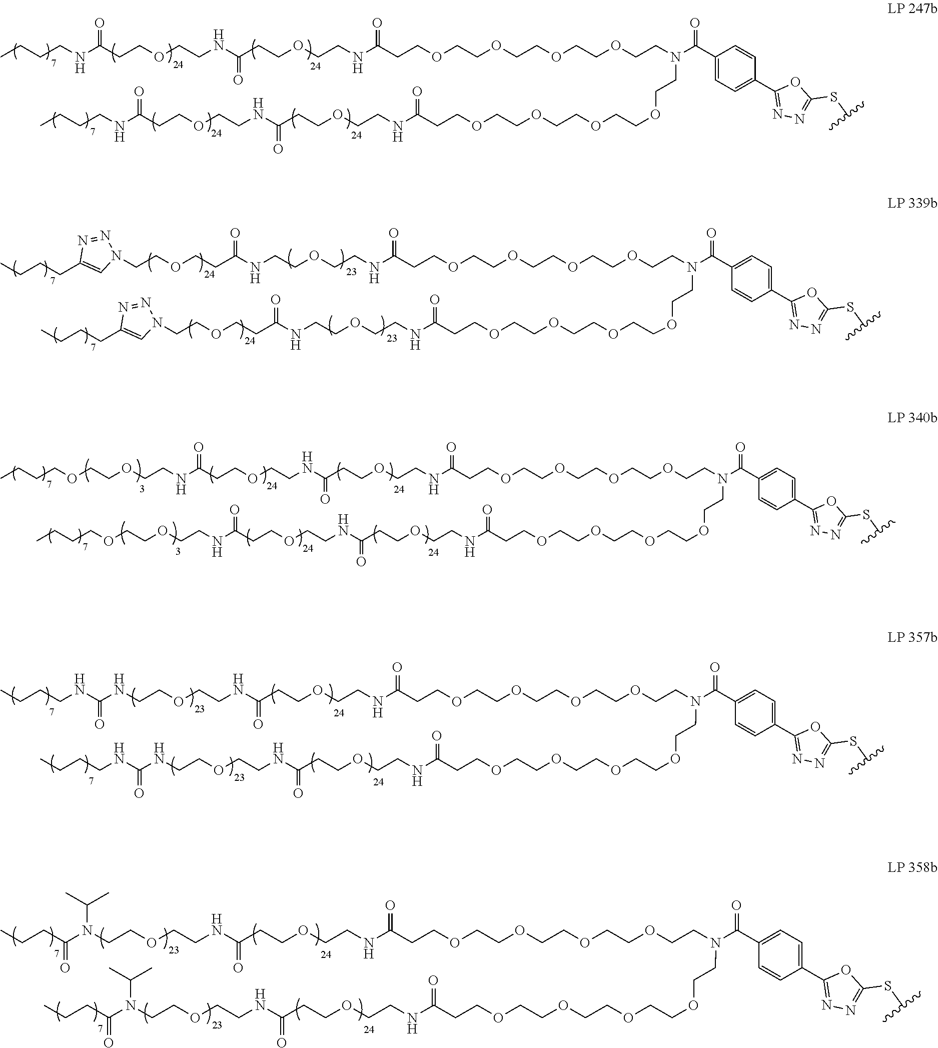
or a pharmaceutically acceptable salt of any of these PK/PD modulators, wherein each [symbol] indicates a point of connection to the RNAi agent.

28. The delivery platform of claim 1, wherein the RNAi agent inhibits expression of the mRNA of a human gene in a skeletal muscle cell.

29. A composition comprising the delivery platform of claim 1.

30. A pharmaceutical composition comprising the composition of claim 29 and a pharmaceutical excipient.

31. A method of treating a disease or disorder of a skeletal muscle cell comprising administering to a subject in need thereof a composition of claim 29.

32. The method of claim 31, wherein the disease or disorder is muscular dystrophy.

33. (canceled)


SEQUENCE LISTING (8 sequences)

SEQ ID NO: 1 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = gm
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatggcc

SEQ ID NO: 2 - RNA, 22 nt, synthetic construct
  modified_base 1: mod_base = gm
  modified_base 2: mod_base = gm
  modified_base 3: mod_base = cm
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methyladenosine
  modified_base 20^21: mod_base = OTHER; note = phosphorothioate linkage
  modified_base 22: mod_base = OTHER; note = 2 -deoxythymidine
  source 1..22: mol_type = other RNA
ggccatgatcttgctgtaacat

SEQ ID NO: 3 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = gm
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatggcc

SEQ ID NO: 4 - RNA, 22 nt, synthetic construct
  modified_base 1: mod_base = gm
  modified_base 2: mod_base = gm
  modified_base 3: mod_base = cm
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2 -deoxythymidine
  source 1..22: mol_type = other RNA
ggccatgatcttgctgtaacat

SEQ ID NO: 5 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = gm
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgttacagcaagatcatggcc

SEQ ID NO: 6 - RNA, 22 nt, synthetic construct
  modified_base 1: mod_base = gm
  modified_base 2: mod_base = gm
  modified_base 3: mod_base = cm
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2 -deoxythymidine
  source 1..22: mol_type = other RNA
ggccatgatcttgctgtaacat

SEQ ID NO: 7 - RNA, 22 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluorocytidine
  modified_base 20^21: mod_base = OTHER; note = phosphorothioate linkage
  modified_base 21: mod_base = cm
  source 1..22: mol_type = other RNA
ctgttacagcaagatcatgacc

SEQ ID NO: 8 - RNA, 22 nt, synthetic construct
  modified_base 1: mod_base = gm
  modified_base 2: mod_base = gm
  modified_base 3: mod_base = um
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methyladenosine
  modified_base 21^22: mod_base = OTHER; note = phosphorothioate linkage
  modified_base 22: mod_base = OTHER; note = 2 -deoxythymidine
  source 1..22: mol_type = other RNA
ggtcatgatcttgctgtaacat